(19) United States
(12) Patent Application Publication
(10) Pub. No.: US 2026/0285882 A1
(43) Pub. Date: Sep. 24, 2026

(54) CHEMICAL COMPOUNDS

(71) Applicant: AN2 Therapeutics, Inc., Menlo Park, CA (US)

(72) Inventors: Chun Yu LIU, Durham, NC (US); Jacob John PLATTNER, Bend, OR (US); Dirk Andries HEERDING, Malvern, PA (US); Matthew Alexander PERRY, Uncasville, CT (US); Peter FARINA, North Salem, NY (US); Xianfeng LI, Cupertino, CA (US); Stephen John BAKER, Collegeville, PA (US); Yasheen ZHOU, Moraga, CA (US); Craig Matthew GOODWIN, Chapel Hill, NC (US); Sarah Ruth JACOBS, Chapel Hill, NC (US); Marissa Caroline AUBREY, Durham, NC (US); Gregory Lee WARREN, Winchester, MA (US); Robert GAMPE, Durham, NC (US)

(73) Assignee: AN2 Therapeutics, Inc., Menlo Park, CA (US)

(21) Appl. No.: 19/165,051

(22) PCT Filed: Mar. 14, 2024
(86) PCT No.: PCT/US2024/020036

§ 371 Date: Sep. 12, 2025

Related U.S. Application Data
(60) Provisional application No. 63/452,355, filed on Mar. 15, 2023.
(60) Provisional application No. 63/524,489, filed on Jun. 30, 2023.
(60) Provisional application No. 63/524,453, filed on Jun. 30, 2023.
(60) Provisional application No. 63/525,623, filed on Jul. 7, 2023.

(51) Int. Cl. C07F 5/02; A61K 31/69; A61P 35/00

(52) U.S. Cl. CPC C07F 5/025; A61K 31/69; A61P 35/00

(57) ABSTRACT

The topological analysis of chiral molecular models led to the development of a general procedure for specifying chirality. The application of this procedure to organic chemical configurations, as described earlier (1956 and before), is retained in this paper, but modified and redefined with regard to certain structure types. These changes are largely based on experience gained at the Beilstein Institute and elsewhere since 1956. The procedure is now extended to allow the treatment of both organic chemical conformations and inorganic chemical configurations up to six ligand atoms. The paper discusses the issues that arise when transferring the symbols obtained from the model's chirality specification to the nomenclature, paying particular attention to the combination of chirality symbols obtained from the general procedure with those obtained from special procedures and with trivial nomenclature systems.

### CROSS-REFERENCE TO RELATED APPLICATIONS

The present application claims the benefit of U.S. Provisional Application Nos. 63/452,355 filed 15 Mar. 2023, 63/524,453 filed 30 Jun. 2023, 63/524,489 filed 30 Jun. 2023, and 63/525,623 filed 7 Jul. 2023, each of which is incorporated herein in its entirety.

### FIELD OF THE INVENTION

The present disclosure provides novel boron compounds or their salts, compositions containing them, and their medical uses. The compounds are active as kinase inhibitors.

### BACKGROUND OF THE INVENTION

The activity of cells may be regulated by external signals that stimulate or inhibit intracellular events. The process by which stimulatory or inhibitory signals are transmitted into and within a cell to elicit an intracellular response is referred to as signal transduction. Over the past decades, cascades of signal transduction events have been elucidated and found to play a central role in a variety of biological responses. Defects in various components of signal transduction pathways have been found to account for a vast number of diseases, including numerous forms of cancer, inflammatory disorders, metabolic disorders, vascular and neuronal diseases (Gaestel et al. Current Medicinal Chemistry (2007) 14:2214-2234).

Kinases represent a class of important signaling molecules. Kinases may generally be classified into protein kinases and lipid kinases, and certain kinases exhibit dual specificities.

Protein kinases are enzymes that phosphorylate other proteins and/or themselves (i.e., autophosphorylation). Protein kinases can be generally classified into three major groups based upon their substrate utilization: tyrosine kinases which predominantly phosphorylate substrates on tyrosine residues (e.g., erb2, PDGF receptor, EGF receptor, VEGF receptor, src, abl), serine/threonine kinases which predominantly phosphorylate substrates on serine and/or threonine residues (e.g., mTorC1, mTorC2, ATM, ATR, DNA-PK, Akt), and dual-specificity kinases which phosphorylate substrates on tyrosine, serine and/or threonine residues.

Lipid kinases are enzymes that catalyze the phosphorylation of lipids within cells. These enzymes, and the resulting phosphorylated lipids and lipid-derived biologically active organic molecules, play a role in many different physiological processes, including cell proliferation, migration, adhesion, and differentiation. A particular group of lipid kinases comprises membrane lipid kinases, i.e., kinases that catalyze the phosphorylation of lipids contained in or associated with cell membranes. Examples of such enzymes include phosphinositide(s) kinases (such as PB-kinases, PI4-Kinases), diacylglycerol kinases, and sphingosine kinases.

The PIK3CA (phosphatidylinositol-4,5-bisphosphate 3-kinase catalytic subunit alpha) gene provides instructions for making the p110 alpha (p110α) protein, which is one piece (subunit) of an enzyme called phosphatidylinositol 3-kinase (PI3K). The p110α protein is called the catalytic subunit because it performs the action of PI3K, while the other subunit (produced by a different gene) regulates the enzyme's activity.

Phosphatidylinositol 3 kinase (PI3K) is a key molecule in the initiation of signal transduction pathways after the binding of extracellular signals to cell surface receptors. An intracellular kinase, PI3K activates multiple intracellular signaling pathways that affect cell growth, proliferation, migration, secretion, differentiation, transcription and translation. Dysregulation of PI3K activity, and aberrant PI3K signaling, lead to a broad range of human diseases, such as cancer, immune disorders, diabetes, and cardiovascular diseases. The PI3K signaling pathway is one of the most highly mutated systems in human cancers.

PI3K signaling is involved in many other disease states including allergic contact dermatitis, rheumatoid arthritis, osteoarthritis, inflammatory bowel diseases, chronic obstructive pulmonary disorder, psoriasis, multiple sclerosis, asthma, disorders related to diabetic complications, and inflammatory complications of the cardiovascular system such as acute coronary syndrome.

PI3K is a member of a unique and conserved family of intracellular lipid kinases that phosphorylate the 3′-OH group on phosphatidylinositols or phosphoinositides. The class I PI3Ks are typically activated by tyrosine kinases or G-protein coupled receptors, and phosphorylate PIP2 to generate PIP3, which engages downstream effectors such as those in the pathways of Akt/PDKI, mTOR, the Tee family kinases, and the Rho family GTPases. The class II and III play a key role in intracellular trafficking through the synthesis of P1(3) P and P1(3,4)P2.

PI3Ks phosphorylate the 3′;-hydroxyl group of phosphatidylinositides (PtdIns). They are divided into three classes based on their structures and substrate specificities. In mammals, class I PI3Ks are further divided into subclasses IA and IB based on their modes of regulation. Class IA PI3Ks are heterodimers of a p110 catalytic subunit and a p85 regulatory subunit. The genes PIK3CA, PIK3CB, and PIK3CD respectively encode three highly homologous class IA catalytic isoforms: p110α, p110β, and p110δ. These isoforms associate with any of five regulatory isoforms, p85α (and its splicing variants p55α and p50α, encoded by PIK3R1), p85β (PIK3R2), and p55γ (PIK3R3), collectively called p85 type regulatory subunits. Class IB PI3Ks are heterodimers of a p110γ catalytic subunit (encoded by PIK3CG) coupled with regulatory isoforms p101 (PIK3R5) or p87 (p84 or p87^{PIKAP}, encoded by PIK3R6). While p110α and p110β are ubiquitously expressed, p110δ and p110γ expression is largely restricted to leukocytes. Initial PI3K-directed drug discovery consisted largely of non-isoform-selective pan-PI3K inhibitors. More recent studies, however, have demonstrated that different PI3K isoforms play divergent roles in cellular signaling and cancer, suggesting that inhibitors targeting individual isoforms may be able to achieve greater therapeutic efficacy. Isoform-selective inhibitors are now emerging. See, Thorpe et al., PI3K in Cancer: Divergent Roles of Isoforms, Modes of Activation, and Therapeutic Targeting, Nat. Rev. Cancer, 2015, 15 (1): 7-24,

Overactivation of the PI3K pathway is one of the most frequent events in human cancers. PIK3CA mutation has been established as causative in many cancer types. Mutations in the gene coding for an isoform are point mutations clustered within several hotspots in helical and kinase domains. Missense mutations occur in all domains of p110g, but the majority cluster in two hotspots, the most common being E542K and E545K in the helical domain and H1047R in the kinase domain. Cell-based analyses confirmed that these hotspot mutations confer transformation via constitutive activation of p110α. Because of the high rate of mutations, targeting of this pathway may provide valuable therapeutic opportunities.

Genetic alterations in gene signaling are believed to be involved in a range of cancers such as endometrial cancer, breast cancer, esophageal squamous-cell cancer, cervical squamous-cell carcinoma, cervical adenocarcinoma, colorectal adenocarcinoma, bladder urothelial carcinoma, glioblastoma, ovarian cancer, non-small-cell lung cancer, esophagogastric cancer, nerve-sheath tumor, head and neck squamous-cell carcinoma, melanoma, esophagogastric adenocarcinoma, soft-tissue sarcoma, prostate cancer, fibrolamellar carcinoma, hepatocellular carcinoma, diffuse glioma, colorectal cancer, pancreatic cancer, cholangiocarcinoma, B-cell lymphoma, mesothelioma, adrenocortical carcinoma, renal non-clear-cell carcinoma, renal clear-cell carcinoma, germ-cell carcinoma, thymic tumor, pheochromocytoma, miscellaneous neuroepithelial tumor, thyroid cancer, leukemia, and encapsulated glioma (Goncalves M D, Hopkins B D, Cantley L C. Phosphatidylinositol 3-Kinase, Grm.vth Disorders, and Cancer. N Engl J Med.2018 Nov. 22; 379 (21): 2052-2062).

The alpha isoform has been implicated, for example, in a variety of human cancers. Angiogenesis has been shown to selectively require the alpha isoform in the control of endothelial cell migration. (Graupera et al, Nature 2008; 453; 662-6). Mutations in the gene coding for PI3Ka or mutations which lead to hyperactivation of PI3Ka are believed to occur in many human cancers such as lung, stomach, endometrial, ovarian, bladder, breast, colon, brain, prostate, and skin cancers. Mutations in the gene coding are point mutations clustered within several hotspots in helical and kinase domains, such as H1047R, E545K and E542K. Many of these mutations have been shown to be oncogenic gain-of-function mutations. Because of the high rate of mutations, targeting of this protein may provide valuable therapeutic opportunities including cancer. While other isoforms are expressed primarily in hematopoietic cells, PI3Ka is expressed constitutively.

Due to the central role of PI3K in regulating organismal glucose homeostasis, inhibition in patients often gives rise to hyperglycemia and/or hyperinsulinemia (Busaidy N L, et al, Management of metabolic effects associated with anticancer agents targeting the PBK-Akt-mTOR pathway. J Clin Oneal 2012; 30:2919-28). High levels of circulating insulin could potentially be mitogenic and/or antiapoptotic for cancer cells and thus negate the antiproliferative effects of inhibitors (Blouin M-J, et al, Abstract 4615: the hyperinsulinemia caused by inhibitors attenuates their antineoplastic efficacy, but can be minimized by co-administration of metformin. Cancer Res 2013; 73:4615).

In the setting of cancer with mutated PI3Kα, one way to overcome the problem of compensatory production of insulin and/or glucose upon systemic inhibition caused by inhibition of the patient's wild-type PI3K would be to develop inhibitors with enhanced selectivity for mutant over wild-type. This would create an increased window for drug dosing to selectively inhibit the pathologic signaling of mutant varients in the cancer cells without affecting the wild-type in the host tissues that control systemic metabolism (Okkenhaug K, Graupera M, Vanhaesebroeck B. Targeting PBK in Cancer: Impact on Tumor Cells, Their Protective Strama, Angiogenesis, and Immunotherapy. Cancer Discov. 2016 October; 6 (10): 1090-1105), thus limiting toxicities and permitting higher doses and more complete inhibition of the drug target (Ariella B. Hanker, et al, Challenges for the clinical development of PBK inhibitors: Strategies to improve their impact in solid tumors. Cancer Discov. 2019 April; 9 (4): 482-491).

Currently PI3K inhibitors are nearly equipotent to wild-type and mutant, such as PI3Kα. Mutant selective PI3Kα inhibitors have been elusive due to the PI3Kα mutations location distal to the active site. As such, inhibitors which target a second, peripheral, binding pocket, with potential differential activity for mutant over wild-type (e.g., H1047R) may provide a route to selective PI3Kα inhibition. Thus, targeting a mutated, peripheral binding pocket of PI3Ka, may in turn provide a valuable therapeutic target for drug development.

As such, kinases, for example lipid kinases such as PI3Ks, are prime targets for drug development. The present disclosure provides a new class of kinase inhibitors.

### SUMMARY OF THE INVENTION

One embodiment of the present disclosure includes a compound of Formula (A):

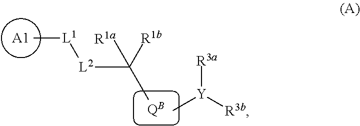

or a tautomer, enantiomer, diastereomer, mixture, salt, hydrate, solvate, or deuterated form thereof,
wherein
A1 is selected from the group consisting of:

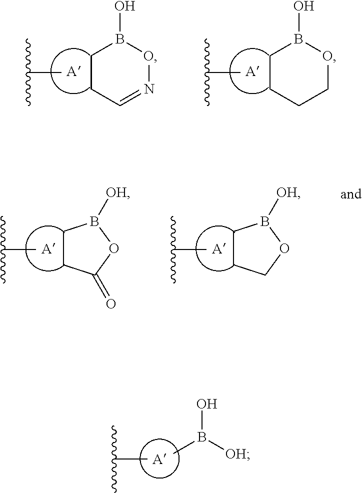

each [symbol] represents the point of attachment to the depicted L¹;
each depicted A′ independently is selected from cycloalkyl, heterocyclyl, aryl, and heteroaryl;
wherein each of heterocyclyl and heteroaryl may contain one or more heteroatoms selected from N, O, and S;
each A1 is optionally substituted;
L¹ is selected from the group consisting of (i) a direct bond, (ii) optionally substituted arylene, and (iii) optionally substituted heteroarylene;
L² is selected from the group consisting of O and NH;
each of R^{1a} and R^{1b} independently is selected from the group consisting of H, halogen, CN, C₁₋₆ alkyl, C₂₋₆ alkenyl, C₂₋₆ alkynyl, C₁₋₆ haloalkyl, and C₁₋₆ alkoxy;
Q^{B} an optionally substituted 8- to 14-membered fused ring system optionally containing one or more heteroatoms selected from the group consisting of O, N, and S; and
Y is H, CR^{3c}, N, O, or S;
when Y is H, each of R^{3a} and R^{3b} is absent;
when Y is N,
each of R^{3a} and R^{3b} independently is selected from the group consisting of H, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl; or
Y, as an N atom, combines with R^{3a} and R^{3b} to form a heterocyclic or heteroaromatic mono ring or spiro or fused ring system, which may contain one or more additional heteroatoms selected from the group consisting of N, O, and S, and wherein the ring or ring system is optionally substituted; when Y is CR^{3c}
R^{3c} is absent or is selected from the group consisting of hydrogen, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl;
each of R^{3a} and R^{3b} independently is selected from the group consisting of H, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl; or
Y, as an C atom, combines with R^{3a} and R^{3b} to form a cycloalkyl, aryl, heterocyclic, or heteroaromatic mono ring or spiro or fused ring system, where the heterocyclic and heteroaromatic rings contain one or more heteroatoms selected from the group consisting of N, O, and S, and wherein the Y-containing ring or ring system is optionally substituted; or
when Y is O or S, R^{3a} is absent and R^{3b} is selected from the group consisting of H, C₁₋₆ alkyl, C₂₋₆ alkenyl, and C₂₋₆ alkynyl.

One aspect of the present disclosure includes wherein A1 is selected from:

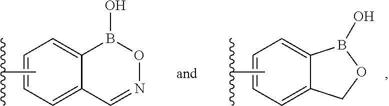

wherein
each A1 is optionally substituted from either depicted ring.

As provided in more detail herein and as appreciated by those skilled in the art, embodiments of the present invention may include a boron atom incorporated into a ring. Metabolic oxidation or hydrolysis may form alternative products (II) or (III). Species (II) may be in equilibrium with the parent compound (I). The scope of the present disclosure is intended to capture all forms. A representative example is shown below:

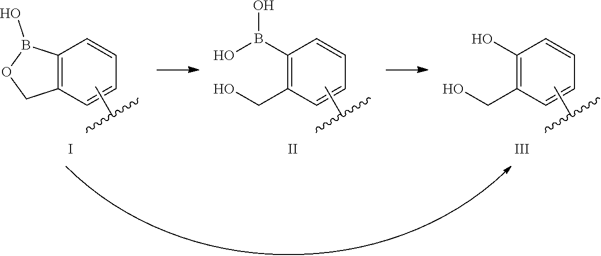

As provided in more detail herein and as appreciated by those skilled in the art, embodiments of the present invention may include an isotopomer or isotopic isomer, wherein one or more atom of a compound of the present disclosure is replaced with an isotope, such as deuterium for hydrogen or ¹³C for carbon. The scope of the present disclosure is intended to capture isotopic forms of the compounds.

One aspect of the present disclosure includes wherein each RAI independently is selected from halogen, OH, C₁₋₆ alkyl, C₂₋₆ alkenyl, C₂₋₆ alkynyl, C₁₋₆ haloalkyl, C₂₋₆ haloalkenyl, C₂₋₆ haloalkynyl, (CH₂)₀₋₆CN, (CH₂)₁₋₆OH, (CH₂)₀₋₆O—C₁₋₆ alkyl, (CH₂)₀₋₆O—C₂₋₆ alkenyl, (CH₂)₀₋₆O—C₂₋₆ alkynyl, (CH₂)₀₋₆C(O)H, (CH₂)₀₋₆C(O) (C₁₋₆ alkyl, C₂₋₆ alkenyl, or C₁₋₆ alkynyl), (CH₂)₀₋₆C(O)OH, (CH₂)₀₋₆C(O)O(C₁₋₆ alkyl, C₂₋₆ alkenyl, or C₂₋₆ alkynyl), (CH₂)₀₋₆NH₂, (CH₂)₀₋₆NH(C₁₋₆ alkyl, C₂₋₆ alkenyl, or C₂₋₆ alkynyl), (CH₂)₀₋₆N(C₁₋₆ alkyl, C₂₋₆ alkenyl, or C₂₋₆ alkynyl)₂, (CH₂)₀₋₆C(O)NH₂, (CH₂)₀₋₆C(O)NH(C₁₋₆ alkyl, C₂₋₆ alkenyl, or C₂₋₆ alkynyl), (CH₂)₀₋₆C(O)N(C₁₋₆ alkyl, C₂₋₆ alkenyl, or C₂₋₆ alkynyl)₂, (CH₂)₀₋₆NHC(O)H, (CH₂)₀₋₆NHC(O)(C₁₋₆ alkyl, C₂₋₆ alkenyl, or C₂₋₆ alkynyl), and (CH₂)₀₋₆N(C₁₋₆ alkyl, C₂₋₆ alkenyl, and C₂₋₆ alkynyl)C(O) (C₁₋₆ alkyl, C₂₋₆ alkenyl, or C₂₋₆ alkynyl), or an isotopic form of R^{A1}.

The recited substituent may be substituted from either depicted ring of A1, and the concept is extended to the more specific recited groups identified in a comparable position, where an optional substituent may be substituted as available throughout the ring or ring system.

One aspect of the present disclosure includes wherein A1 is selected from the group consisting of:

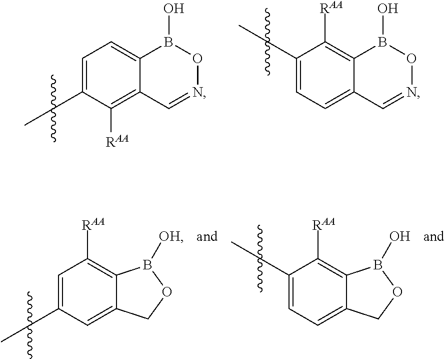

each A1 is optionally further substituted.

One aspect of the present disclosure includes wherein each R^{AA} is independently selected from the group consisting of H, halogen, CN, C₁₋₆ alkyl, C₂₋₆ alkenyl, C₂₋₆ alkynyl, C₁₋₆ haloalkyl, C₂₋₆ haloalkenyl, C₂₋₆ haloalkynyl, OH, O—C₁₋₆ alkyl, O—C₂₋₆ alkenyl, and O—C₂₋₆ alkynyl.

For clarity, when R^{AA} is hydrogen, the depicted R^{AA} is absent and A1 is not further substituted.

One aspect of the present disclosure includes wherein one or more atom is an isotopic form.

One aspect of the present disclosure includes wherein the isotope is deuterium.

One aspect of the present disclosure includes wherein R^{AA} is deuterated.

One aspect of the present disclosure includes wherein R^{AA} is selected from the group consisting of C₁₋₆ alkyl, O—C₁₋₆ alkyl, C₁₋₆ haloalkyl, deuterated C₁₋₆ alkyl, deuterated O—C₁₋₆ alkyl, deuterated C₁₋₆ haloalkyl, halogen, and CN.

One aspect of the present disclosure includes wherein R^{AA} is F, Cl, CH₃, OCH₃, or CD₃.

One aspect of the present disclosure includes wherein Q^{B} is selected from the group consisting of:

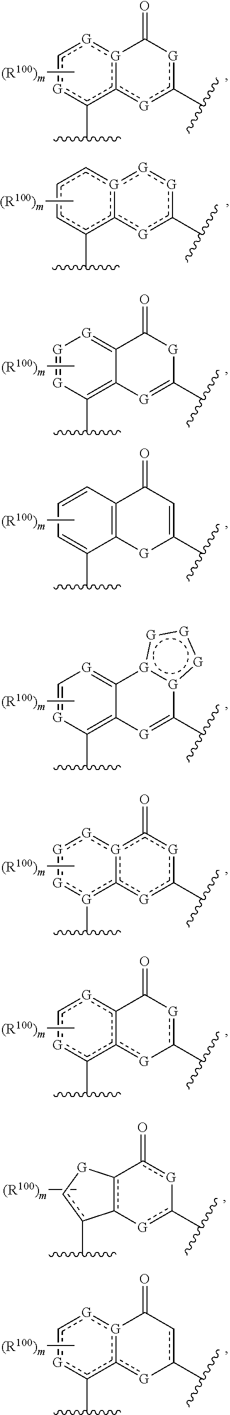

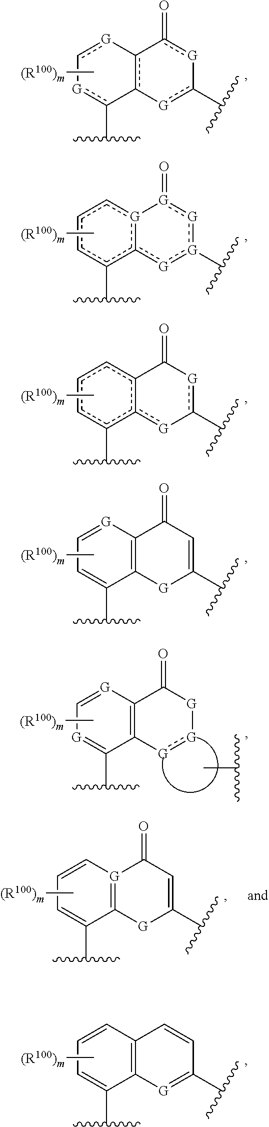

wherein
each G is, at each occurrence, independently selected from carbon or a heteroatom selected from O, N, or S;
each m is, at each occurrence, independently selected from 0, 1, 2, 3, 4, 5, and 6;
when m is not 0, each R¹⁰⁰ may be substituted from any depicted ring; and
when present, each R¹⁰⁰ independently is selected from the group consisting of halogen, OH, oxo, C₁₋₆alkyl, C₂₋₆ alkenyl, C₂₋₆ alkynyl, C₁₋₆ haloalkyl, C₁₋₆ alkoxy, (CH₂)_{q}—N(H or C₁₋₆alkyl), (CH₂)_{q}—O—C(O)—(CH₂)ᵣ—R¹⁴⁰, (CH₂)_{q}—NH—C(O)—(CH₂)ᵣ—R¹⁴⁰, (CH₂)_{q}—O—C(O)—(CH₂)ᵣ—OR¹⁴⁰, (CH₂)_{q}—NH—C(O)—(CH₂)ᵣ—OR¹⁴⁰, (CH₂)_{q}—O—(CH₂)ᵣ—R¹⁴⁰, (CH₂)_{q}—NH—(CH₂)ᵣ—R¹⁴⁰, (CH₂)_{q}—O—(CH₂)ᵣ—OR¹⁴⁰, (CH₂)_{q}—NH—(CH₂)ᵣ—OR¹⁴⁰, C₃₋₁₀cycloalkyl, (CH₂)_{q}-heterocycle, (CH₂)_{q}-aryl, and (CH₂)_{q}-heteroaryl, wherein each of the cycloalkyl, heterocycle, aryl, and heteroaryl is optionally substituted with one or more halogen, OH, C₁₋₆ alkyl, C₁₋₆haloalkyl, C₁₋₆ alkoxy, and C₁₋₆ haloalkoxy;
R¹⁴⁰ is an E3 ligase binding ligand; and
each of q and r, independently is, at each occurrence, selected from 0, 1, 2, 3, 4, 5, and 6.

One aspect of the present disclosure includes wherein Q^{B} is selected from the group consisting of:

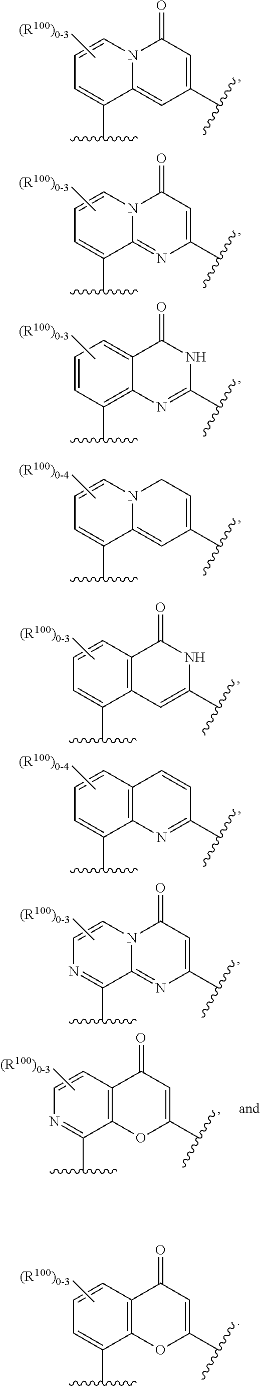

One aspect of the present disclosure includes wherein Q^{B} is:

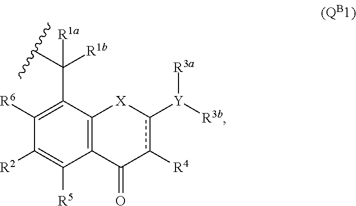

wherein
X is C(R^{x})₂, O, NR^{x}, or S;
each R^{x} independently is selected from the group consisting of H, C₁₋₆ alkyl, C₂₋₆ alkenyl, and C₂₋₆ alkynyl;
R² is selected from the group consisting of H, halogen, CN, C₁₋₆ alkyl, C₂₋₆ alkenyl, C₂₋₆ alkynyl, C₁₋₆haloalkyl, C₂₋₆ haloalkenyl, C₂₋₆ haloalkynyl, C₁₋₆ alkoxy, (CH₂)₀₋₆—R^{QB1}, (CH₂)₀₋₆—OR^{QB1}, (CH₂)₀₋₆—N(R^{QB1})₂, (CH₂)₀₋₆C(O)R^{QB1}, (CH₂)₀₋₆C(O)OR^{QB1}, (CH₂)₀₋₆C(O)N(R^{QB1})₂, (CH₂)₀₋₆—C₃₋₁₀ cycloalkyl, (CH₂)₀₋₆-aryl, (CH₂)₀₋₆-heterocycle, and (CH₂)₀₋₆-heteroaryl;
R⁴ is selected from the group consisting of H, halogen, CN, C₁₋₆ alkyl, C₂₋₆ alkenyl, C₂₋₆ alkynyl, C₁₋₆ haloalkyl, C₂₋₆ haloalkenyl, C₂₋₆ haloalkynyl, C₁₋₆ alkoxy, (CH₂)₀₋₆—R^{QB1}, (CH₂)₀₋₆—OR^{QB1}, (CH₂)₀₋₆—N(R^{QB1})₂, (CH₂)₀₋₆C(O)R^{QB1}, (CH₂)₀₋₆C(O)OR^{QB1}, (CH₂)₀₋₆C(O)N(R^{QB1})₂, (CH₂)₀₋₆—C₃₋₁₀ cycloalkyl, (CH₂)₀₋₆-aryl, (CH₂)₀₋₆-heterocycle, and (CH₂)₀₋₆-heteroaryl;
R⁵ is selected from the group consisting of H, halogen, CN, C₁₋₆ alkyl, C₂₋₆ alkenyl, C₂₋₆ alkynyl, C₁₋₆ haloalkyl, C₂₋₆ haloalkenyl, C₂₋₆ haloalkynyl, C₁₋₆ alkoxy, (CH₂)₀₋₆—R^{QB1}, (CH₂)₀₋₆—OR^{QB1}, (CH₂)₀₋₆—N(R^{QB1})₂, (CH₂)₀₋₆—C(O)R^{QB1}, (CH₂)₀₋₆C(O)OR^{QB1}, (CH₂)₀₋₆C(O)N(R^{QB1})₂, (CH₂)₀₋₆—C₃₋₁₀ cycloalkyl, (CH₂)₀₋₆-aryl, (CH₂)₀₋₆-heterocycle, and (CH₂)₀₋₆-heteroaryl;
R⁶ is selected from the group consisting of H, halogen, CN, C₁₋₆ alkyl, C₂₋₆ alkenyl, C₂₋₆ alkynyl, C₁₋₆ haloalkyl, C₂₋₆ haloalkenyl, C₂₋₆ haloalkynyl, C₁₋₆ alkoxy, (CH₂)₀₋₆—R^{QB1}, (CH₂)₀₋₆—OR^{QB1}, (CH₂)₀₋₆—N(R^{QB1})₂, (CH₂)₀₋₆—C(O)R^{QB1}, (CH₂)₀₋₆—C(O)OR^{QB1}, (CH₂)₀₋₆—C(O)N(R^{QB1})₂, (CH₂)₀₋₆—C₃₋₁₀ cycloalkyl, (CH₂)₀₋₆-aryl, (CH₂)₀₋₆-heterocycle, and (CH₂)₀₋₆-heteroaryl; and each R^{QB1} is independently H, halogen, OH, C₁₋₆ alkyl, C₂₋₆ alkenyl, C₂₋₆ alkynyl, C₁₋₆ haloalkyl, C_{20.6} haloalkenyl, C₂₋₆ haloalkynyl, C₁₋₆ alkoxy, C₂₋₆ alkenoxy, C₂₋₆ alkynoxy, C₁₋₆ haloalkoxy, C₂₋₆ haloalkenoxy, and C₂₋₆ alkynoxy.

One aspect of the present disclosure includes wherein the compound is a compound of Formula A(Ia) or A(Ib):

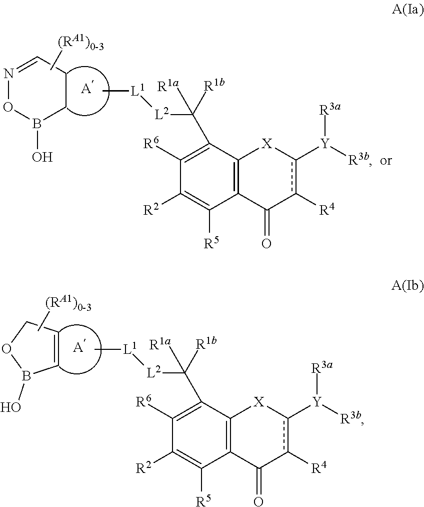

One aspect of the present disclosure includes wherein A′ is fused aryl or heteroaryl.

One aspect of the present disclosure includes wherein A′ is selected from optionally substituted phenyl.

One aspect of the present disclosure includes wherein A′ is substituted phenyl.

One aspect of the present disclosure includes wherein L¹ is selected from the group consisting of a direct bond, optionally substituted heteroarylene, or optionally substituted arylene.

One aspect of the present disclosure includes wherein optionally substituted arylene is optionally substituted phenylene.

One aspect of the present disclosure includes wherein optionally substituted heteroaylene is optionally substituted pyridylene.

One aspect of the present disclosure includes wherein each of optionally substituted arylene and optionally substituted heteroarylene is unsubstituted.

One aspect of the present disclosure includes wherein each of optionally substituted arylene and optionally substituted heteroarylene is substituted with one or two substitutents selected from the group consisting of halogen, C₁-C₃ alkyl, C₁-C₃ haloalkyl, C₂-C₃ alkenyl, C₂-C₃ haloalkenyl, C₂-C₃ alkynyl, C₂-C₃ haloalkynyl, OH, O—(C₁₋₃ alkyl, C₂₋₃ alkenyl, C₂₋₃ alkynyl), NH₂, NH(C₁₋₃ alkyl, C₂₋₃ alkenyl, C₂₋₃ alkynyl), N(C₁₋₃ alkyl, C₂₋₃ alkenyl, C₂₋₃ alkynyl)₂, C₃ cycloalkyl, and C₃ halocycloalkyl.

The compound of claim 22, wherein each of optionally substituted arylene and optionally substituted heteroarylene is substituted with one or two substitutents selected from the group consisting of halogen and C₁-C₃ alkyl.

One aspect of the present disclosure includes wherein L² is NH.

One aspect of the present disclosure includes wherein each of R^{1a} and R^{1b} independently is selected from the group consisting of H, halogen, CN, C₁₋₃ haloalkyl, and C₁₋₃ alkyl.

One aspect of the present disclosure includes wherein each of R^{1a} and R^{1b} independently is selected from the group consisting of H and CH₃.

One aspect of the present disclosure includes wherein the depicted dashed bond is a double bond.

Portions of one or more embodiments of the present disclosure may be incorporated as disclosed herein to create a compound of the present disclosure. Reference is hereby made to the synthetic teaching and examples disclosed of one or more of the following patent publications: WO 2024/026423, WO 2024/008122, WO 2024/000401, WO 2023/239710, WO 2023/230262, WO 2023/207881, WO 2023/205680, WO 2023/192416, WO 2023/159155, WO 2023/081209, WO 2023/078401, WO 2023/060262, WO 2024/026419, WO 2024/026424, and WO 2021/202964, each of which is incorporated herein with regard to the synthetic teaching. In certain embodiments, a portion, Q^{B}, may be selected from the noted publications. In certain embodiments, a portion, Y^{T}, may be selected from the noted publications. For example, a portion, of the depicted Y(R^{3a})(R^{3b}), may be selected from the noted publications.

As provided in more detail herein and as appreciated by those skilled in the art, Y^{T} may be selected and a compound of the present disclosure may be prepared to enhance molecular properties (e.g., molecular weight, dipole moment, polarizability, van der Vaals volume, and surface area) or bulk properties (e.g. acidic or basic character in solution, octanol/water partition coefficient, solubility). Moreover, Y^{T} may be selected to enhance hydrogen-bonding capacity and, thereby improved selectivity for a target.

One aspect of the present disclosure includes wherein Y is N or CR³.

One aspect of the present disclosure includes wherein Y is selected from the group consisting of substituted or unsubstituted:

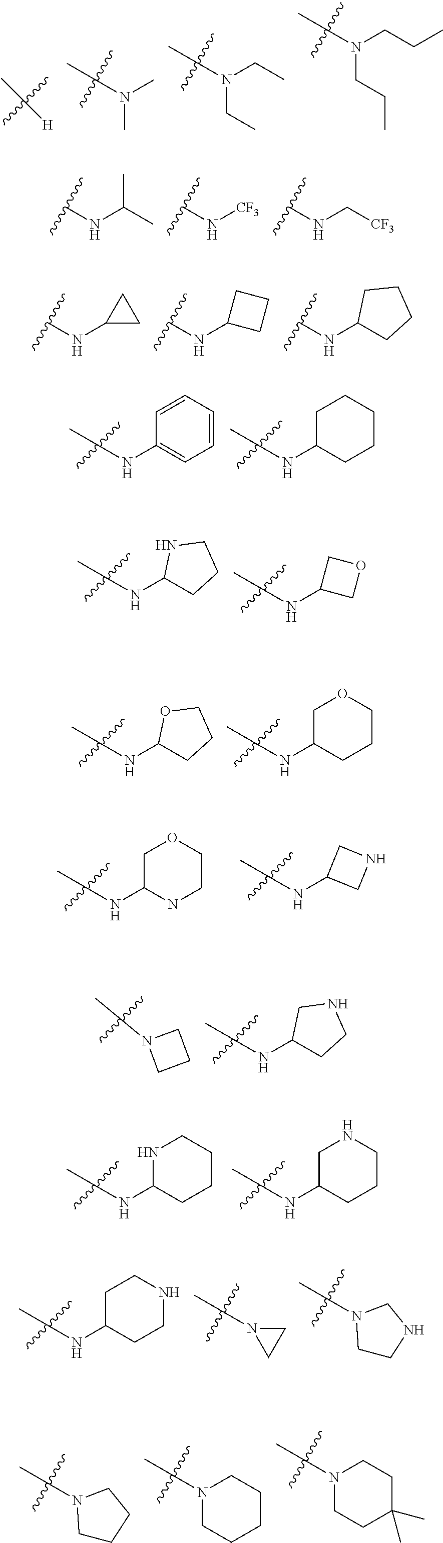

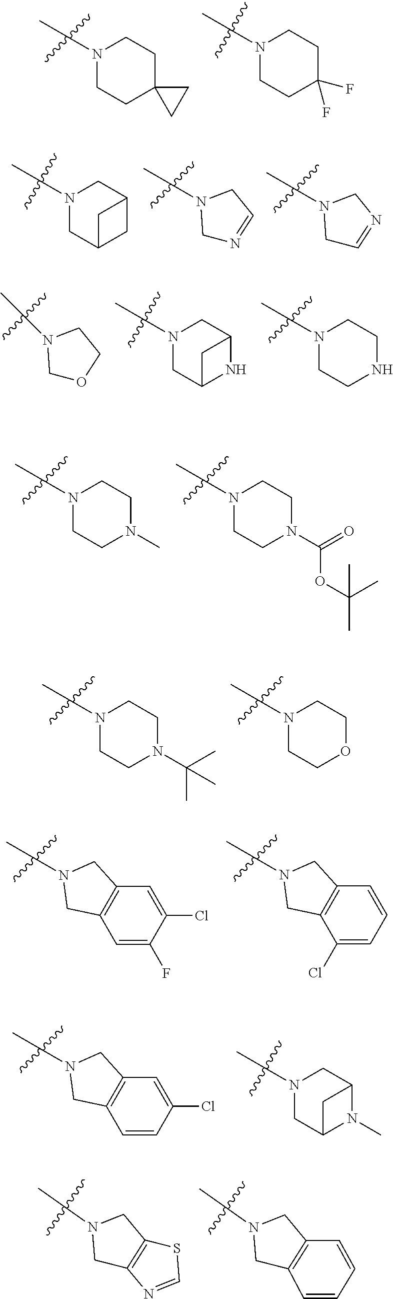

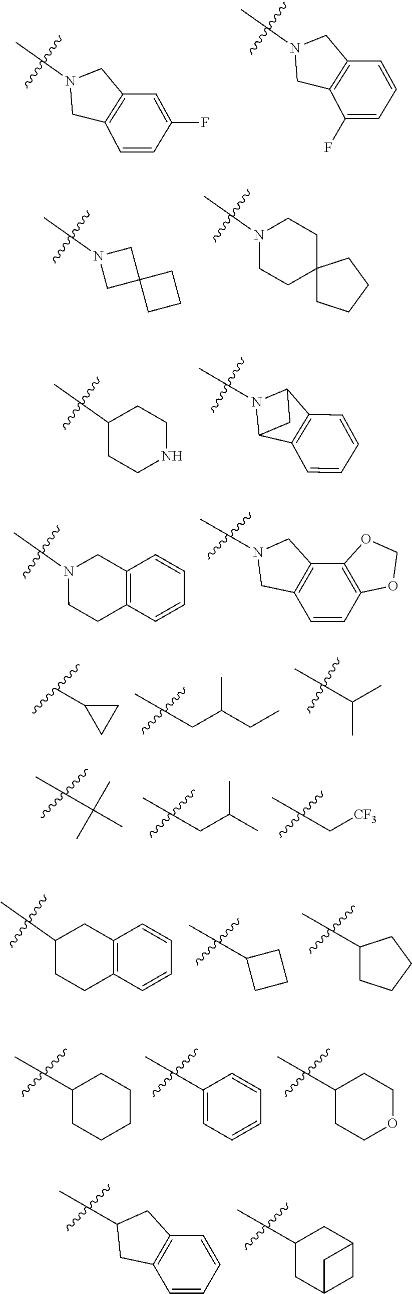

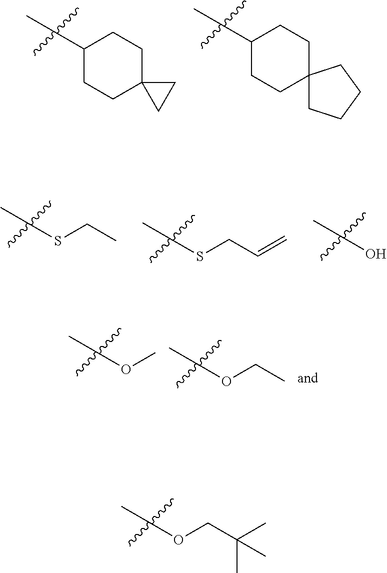

One aspect of the present disclosure includes wherein Y is CR^{3c} is selected from the group consisting of hydrogen, C₁₋₆ alkyl, C₂₋₆ alkenyl, C₂₋₆ alkynyl, C₁₋₆ haloalkyl, C₂₋₆ haloalkenyl, C₂₋₆haloalkynyl, aryl, C₃₋₆ cycloalkyl, and 3 to 6 membered heterocyclyl.

One aspect of the present disclosure includes wherein each of R^{3a} and R^{3b} is independently selected from the group consisting of H, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3- to 6-membered heterocyclyl.

One aspect of the present disclosure includes wherein each of R², R⁴, R⁵, and R⁶ independently is H, halogen, C₁₋₆ alkyl, C₂₋₆ alkenyl, C₂₋₆ alkynyl, C₃-C₆ cycloalkyl, C₂₋₆ haloalkenyl, C₂₋₆ haloalkynyl, or C₁₋₆ haloalkyl.

One aspect of the present disclosure includes wherein R⁴ is H or CH₃.

One aspect of the present disclosure includes wherein R² is H, C₁₋₆ alkyl, C₂₋₆ alkenyl, or C₂₋₆ alkynyl.

One aspect of the present disclosure includes wherein R⁴ is CH₃.

One aspect of the present disclosure includes wherein R⁵ is H.

One aspect of the present disclosure includes wherein R⁶ is H.

One embodiment of the present disclosure includes a compound of Formula (AWH):

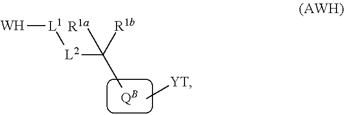

or a tautomer, enantiomer, diastereomer, mixture, salt, hydrate, solvate, or deuterated form thereof,
wherein
WH is a warhead moiety;
L¹ is selected from the group consisting of a direct bond, optionally substituted cycloalkylene, optionally substituted heterocyclylene, optionally substituted arylene, and optionally substituted heteroarylene;
L² is selected from the group consisting of (CH₂)₁₋₆, O, C(O), S, and NH;
each of R^{1a} and R^{1b} independently is selected from the group consisting of H, halogen, CN, C₁₋₆ alkyl, C₂₋₆ alkenyl, C₂₋₆ alkynyl, C₁₋₆ haloalkyl, and C₁₋₆ alkoxy;
or L²-C(R^{1a})(R^{1b}) combines to form NHC(S), C(S)NH, NHC(O), C(O)NH, NHS(O)₂, S(O)₂NH, NHC(NH), or C(NH)NH;
Q^{B} an optionally substituted 8- to 14-membered spiro or fused ring system optionally containing one or more heteroatoms selected from the group consisting of O, N, and S;
YT is any moiety that provides desired physicochemical properties; and
an optional E3 ligase binding ligand.

One aspect of the present disclosure includes wherein the optional E3 ligase binding ligand is connected via a linker.

One aspect of the present disclosure includes wherein WH is a ring system:

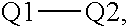

wherein:
the ring system is attached to the depicted L¹ through either the Q1 or Q2 portion; and
Q1 is an optionally substituted 5- or 6-membered ring, having one or more degrees of unsaturation, and optionally having one or more heteroatom selected from B, N, O, or S; and
Q2 is fused to Q1 and is an optionally substituted 5- or 6-membered ring, having one or more degrees of unsaturation, and optionally having one or more heteroatom selected from B, N, O, or S; or
Q1 does not exist; and
Q2 is an optionally substituted 5- or 6-membered ring, having one or more degrees of unsaturation, and optionally having one or more heteroatom selected from B, N, O, or S;
One aspect of the present disclosure includes wherein WH is selected from an optionally substituted:
Group (a): a cyclohexenone derivative, an alkyl halide derivative, a sulfonyl derivative, an α-cyanoenone derivative, and an epoxide or spiro-epoxide derivative;

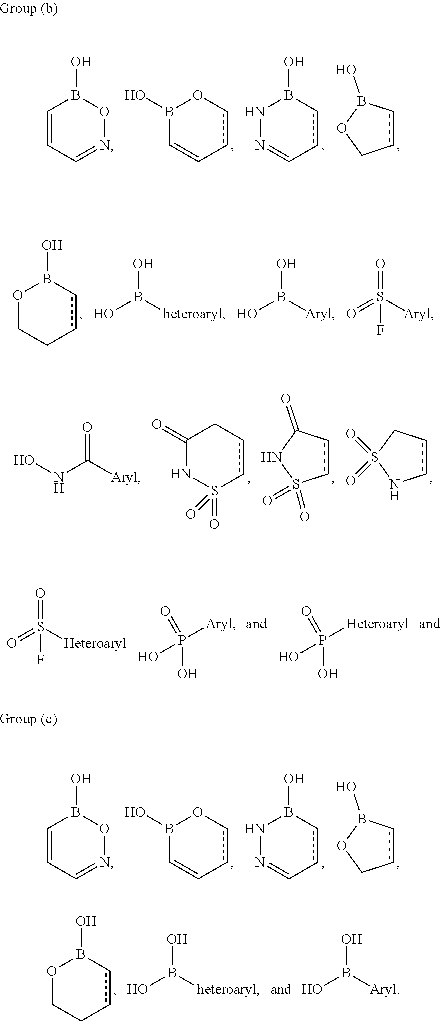

One aspect of the present disclosure includes wherein optionally substituted is substituted with one or more substitutents selected from the group consisting of: deuterium, halogen, haloalkyl, R′, OR′, OH, SH, SR′, NO₂, CN, C(O)R′, NH₂, C(O)OR′, OC(O)R′, CON(R′)₂, OC(O)N(R′)₂, NH₂, NHR′, N(R′)₂, NHCOR′, NHCOH, NHCONH₂, NHCONHR′, NHCON(R′)₂, NRCOR′, NRCOH, NHCO₂H, NHCO₂R′, NHC(S)NH₂, NHC(S)NHR′, NHC(S)N(R′)₂, CO₂R′, CO₂H, CHO, CONH₂, CONHR′, CON(R′)₂, S(O)₂H, S(O)₂R′, SO₂NH₂, S(O)H, S(O)R′, SO₂NHR′, SO₂N(R′)₂, NHS(O)₂H, NR′S(O)₂H, NHS(O)₂R′, NR′S(O)₂R′, Si(R′)₃, ═O, ═S, ═NNHR′, ═NNH₂, ═NN(R′)₂, ═N—OR′, ═N—OH, ═NNHCOR′, ═NNHCOH, =NNHCO₂R′, ═NNHCO₂H, ═NNHSO₂R′, ═NNHSO₂H, ═N—CN, ═NH, ═NR′, where each of the preceding may be linked through a divalent alkylene linker, (CH₂)ₓ, where x is 1, 2, or 3, and where each occurrence of R′ is the same or different and represents hydrogen, alkyl, alkenyl, alkynyl, cycloalkyl, aryl, heterocyclyl, or heteroaryl, or when two R′ are each attached to a nitrogen atom, they may form a saturated or unsaturated heterocyclic ring containing from 4 to 6 ring atoms.

One aspect of the present disclosure includes wherein Q1-Q2 is selected from:

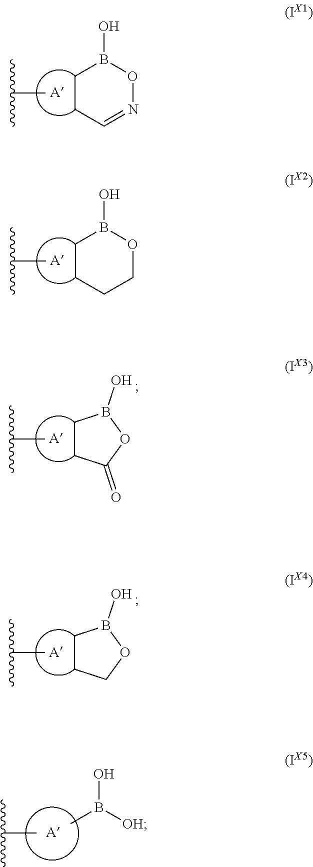

where each [symbol] represents the point of attachment to the depicted L¹;
each depicted A′ is a cycloalkyl, heterocyclyl, aryl, or heteroaryl, mono-ring;
wherein each of the heterocyclyl or heteroaryl rings contain one or more heteroatoms selected from N, O, and S; and each is optionally substituted with one or more R^{A1}, where each R^{A1} independently is selected from halogen, OH, C₁₋₆ alkyl, C₂₋₆ alkenyl, C₂₋₆ alkynyl, C₁₋₆ haloalkyl, C₂₋₆ haloalkenyl, C₂₋₆ haloalkynyl, (CH₂)₀₋₆CN, (CH₂)₁₋₆OH, (CH₂)₀₋₆O—C₁₋₆ alkyl, (CH₂)₀₋₆O—C₂₋₆ alkenyl, (CH₂)₀₋₆O—C₂₋₆ alkynyl, (CH₂)₀₋₆C(O)H, (CH₂)₀₋₆C(O)(C₁₋₆ alkyl, C₂₋₆ alkenyl, or C₁₋₆ alkynyl), (CH₂)₀₋₆C(O)OH, (CH₂)₀₋₆C(O)O(C₁₋₆ alkyl, C₂₋₆ alkenyl, or C₂₋₆ alkynyl), (CH₂)₀₋₆—NH₂, (CH₂)₀₋₆—NH(C₁₋₆ alkyl, C₂₋₆ alkenyl, or C₂₋₆ alkynyl), (CH₂)₀₋₆—N(C₁₋₆ alkyl, C₂₋₆ alkenyl, or C₂₋₆ alkynyl)₂, (CH₂)₀₋₆C(O)NH₂, (CH₂)₀₋₆C(O)NH(C₁₋₆ alkyl, C₂₋₆ alkenyl, or C₂₋₆ alkynyl), (CH₂)₀₋₆C(O)N(C₁₋₆ alkyl, C₂₋₆ alkenyl, or C₂₋₆ alkynyl)₂, (CH₂)₀₋₆—NHC(O)H, (CH₂)₀₋₆—NHC(O)(C₁₋₆ alkyl, C₂₋₆ alkenyl, or C₂₋₆ alkynyl), and (CH₂)₀₋₆—N(C₁₋₆ alkyl, C₂₋₆ alkenyl, and C₂₋₆ alkynyl)C(O)(C₁₋₆ alkyl, C₂₋₆ alkenyl, or C₂₋₆ alkynyl), or an isotopic form of R^{A}.

One aspect of the present disclosure includes wherein A′ is aryl.

One aspect of the present disclosure includes wherein A′ is phenyl.

One aspect of the present disclosure includes wherein A′ is heterocyclyl.

One aspect of the present disclosure includes wherein A′ is heteroaryl.

One aspect of the present disclosure includes wherein heteroaryl is a 5- or 6-membered heteroaryl, containing one or two heteroatoms selected from N, O, and S.

One aspect of the present disclosure includes wherein Q^{B} is an oxo-substituted 6,6 fused ring containing one or more heteroatom selected from O, N, or S.

One aspect of the present disclosure includes wherein Q^{B} is selected from:

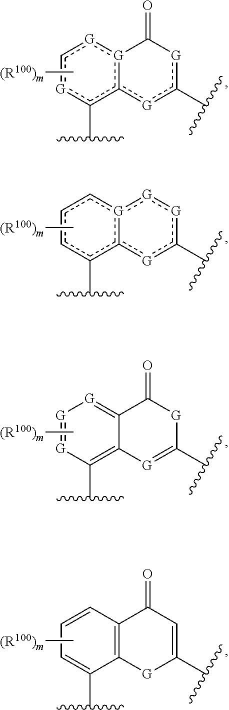

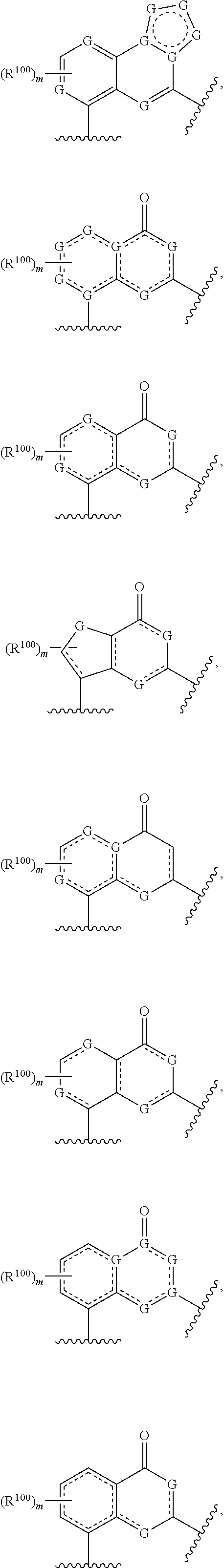

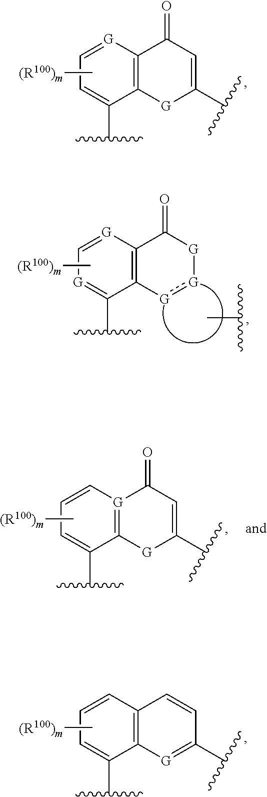

wherein
each G is, at each occurrence, independently selected from carbon or a heteroatom selected from O, N, or S;
each m is, at each occurrence, independently selected from 0, 1, 2, 3, 4, 5, and 6; and
each R¹⁰⁰ independently is selected from the group consisting of halogen, OH, oxo, C₁₋₆ alkyl, C₂₋₆ alkenyl, C₂₋₆ alkynyl, C₁₋₆ haloalkyl, C₂₋₆ haloalkenyl, C₂₋₆ haloalkynyl, C₁₋₆ alkoxy, (CH₂)_{q}—N(H or C₁₋₆alkyl), (CH₂)_{q}0-C(O)—(CH₂)ᵣ—R⁴⁰, (CH₂)_{q}—NH—C(O)—(CH₂)ᵣ—R*, (CH₂)_{q}—O—C(O)—(CH₂)ᵣ—OR¹⁴⁰, (CH₂)_{q}—NH—C(O)—(CH₂)ᵣ—OR¹⁴⁰, (CH₂)_{q}—O—(CH₂)ᵣ—R¹⁴⁰, (CH₂)_{q}—NH—(CH₂)ᵣ—R⁴⁰, (CH₂)_{q}O—(CH₂)ᵣ—OR¹⁴⁰, (CH₂)_{q}—NH—(CH₂)ᵣ—OR¹⁴⁰, C₃₋₁₀ cycloalkyl, (CH₂)_{q}-heterocycle, (CH₂)_{q}-aryl, and (CH₂)_{q}-heteroaryl, wherein each of the cycloalkyl, heterocycle, aryl, and heteroaryl is optionally substituted with one or more halogen, OH, C₁₋₆ alkyl, C₁₋₆ haloalkyl, C₁₋₆ alkoxy, and C₁₋₆ haloalkoxy;
R¹⁴⁰ is an E3 ligase binding ligand; and each of q and r, independently is, at each occurrence, selected from 0, 1, 2, 3, 4, 5, and 6.

One aspect of the present disclosure includes wherein Q^{B} is selected from:

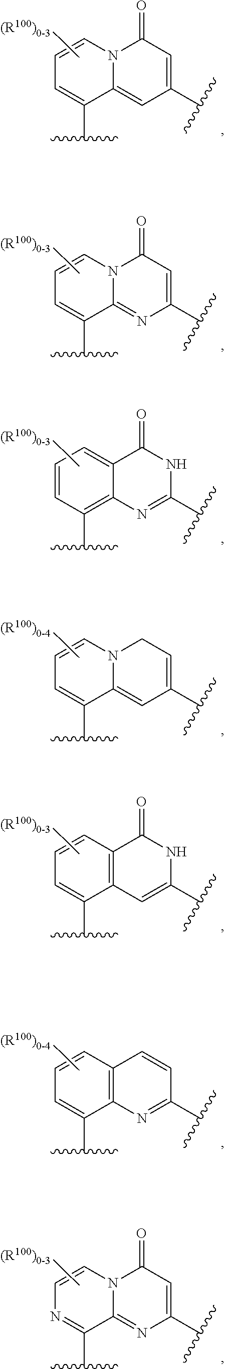

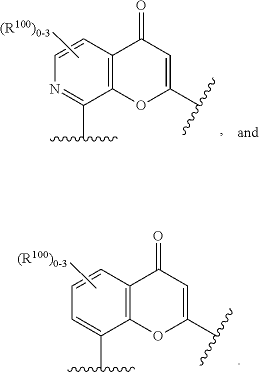

One aspect of the present disclosure includes wherein at least one R¹⁰⁰ is substituted from a depicted N in Q^{B}.

One aspect of the present disclosure includes wherein Y^{T} is YR^{3a}R^{3b}; and
Y is H, CR^{3c}, N, O, or S;
when Y is H, each of R^{3a} and R^{3b} is absent;
when Y is N,
each of R^{3a} and R^{3b} independently is selected from the group consisting of H, C₁₋₆ alkyl, C₂₋₆ alkenyl, C₂₋₆alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl; or
Y, as an N atom, combines with R^{3a} and R^{3b} to form a heterocyclic or heteroaromatic mono ring or spiro or fused ring system, which may contain one or more additional heteroatoms selected from the group consisting of N, O, and S, and wherein the ring or ring system is optionally substituted;
when Y is CR^{3c}
R^{3c} is absent or is selected from the group consisting of hydrogen, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl;
each of R^{3a} and R^{3b} independently is selected from the group consisting of H, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl; or Y, as an C atom, combines with R^{3a} and R^{3b} to form a cycloalkyl, aryl, heterocyclic, or heteroaromatic mono ring or spiro or fused ring system, where the heterocyclic and heteroaromatic rings contain one or more heteroatoms selected from the group consisting of N, O, and S, and wherein the Y-containing ring or ring system is optionally substituted; or when Y is O or S, R^{3a} is absent and R^{3b} is selected from the group consisting of H, C₁₋₆ alkyl, C₂₋₆ alkenyl, and C₂₋₆ alkynyl.

One aspect of the present disclosure includes wherein when YR^{3a}R^{3b} forms a ring, the ring is optionally substituted with one or more substitutents selected from the group consisting of: deuterium, halogen, haloalkyl, R′, OR′, OH, SH, SR′, NO₂, CN, C(O)R′, NH₂, C(O)OR′, OC(O)R′, CON(R′)₂, OC(O)N(R′)₂, NH₂, NHR′, N(R′)₂, NHCOR′, NHCOH, NHCONH₂, NHCONHR′, NHCON(R′)₂, NRCOR′, NRCOH, NHCO₂H, NHCO₂R′, NHC(S)NH₂, NHC(S)NHR′, NHC(S)N(R′)₂, CO₂R′, CO₂H, CHO, CONH₂, CONHR′, CON(R′)₂, S(O)₂H, S(O)₂R′, SO₂NH₂, S(O)H, S(O)R′, SO₂NHR′, SO₂N(R′)₂, NHS(O)₂H, NR′S(O)₂H, NHS(O)₂R′, NR′S(O)₂R′, Si(R′)₃, ═O, ═S, ═NNHR′, ═NNH₂, ═NN(R′)₂, ═N—OR′, ═N—OH, ═NNHCOR′, ═NNHCOH, ═NNHCO₂R′, ═NNHCO₂H, ═NNHSO₂R′, ═NNHSO₂H, ═N—CN, ═NH, ═NR′, where each of the preceding may be linked through a divalent alkylene linker, (CH₂)ₓ, where x is 1, 2, or 3, and where each occurrence of R′ is the same or different and represents hydrogen, alkyl, alkenyl, alkynyl, cycloalkyl, aryl, heterocyclyl, or heteroaryl, or when two R′ are each attached to a nitrogen atom, they may form a saturated or unsaturated heterocyclic ring containing from 4 to 6 ring atoms.

One aspect of the present disclosure includes wherein the compound is a compound of (AWH2):

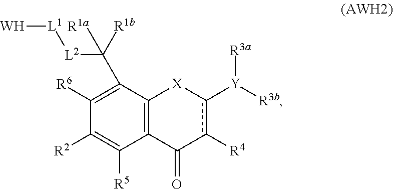

or a tautomer, enantiomer, diastereomer, mixture, salt, hydrate, solvate, or deuterated form thereof, wherein
X is C(R^{x})₂, O, NR^{x}, or S;
each RN independently is selected from the group consisting of H, C₁₋₆ alkyl, C₂₋₆ alkenyl, and C₂₋₆ alkynyl;
R² is selected from the group consisting of H, halogen, CN, C₁₋₆ alkyl, C₂₋₆ alkenyl, C₂₋₆ alkynyl, C₁₋₆ haloalkyl, C₁₋₆ alkoxy, (CH₂)₀₋₆—R^{AWH2}, (CH₂)₀₋₆—OR^{AWH2}, (CH₂)₀₋₆—N(R^{AWH2})₂, (CH₂)₀₋₆—C(O)R^{AWH2} (CH₂)₀₋₆C(O)OR^{AWH2}, (CH₂)₀₋₆C(O)N(R^{AWH2})₂, (CH₂)₀₋₆-C₃₋₁₀ cycloalkyl, (CH₂)₀₋₆-aryl, (CH₂)₀₋₆-heterocycle, and (CH₂)₀₋₆-heteroaryl;
R⁴ is selected from the group consisting of H, halogen, CN, C₁₋₆ alkyl, C₂₋₆ alkenyl, C₂₋₆ alkynyl, C₁₋₆ haloalkyl, C₁₋₆ alkoxy, (CH₂)₀₋₆—R^{AWH2}, (CH₂)₀₋₆—OR^{AWH2}, (CH₂)₀₋₆—N(R^{AWH2})₂, (CH₂)₀₋₆—C(O)R^{AWH2} (CH₂)₀₋₆—C(O)OR^{AWH2}, (CH₂)₀₋₆C(O)N(R^{AWH2})₂, (CH₂)₀₋₆—C₃₋₁₀ cycloalkyl, (CH₂)₀₋₆-aryl, (CH₂)₀₋₆-heterocycle, and (CH₂)₀₋₆-heteroaryl;
R⁵ is selected from the group consisting of H, halogen, CN, C₁₋₆ alkyl, C₂₋₆ alkenyl, C₂₋₆ alkynyl, C₁₋₆ haloalkyl, C₁₋₆ alkoxy, (CH₂)₀₋₆—R^{AWH2}, (CH₂)₀₋₆OR^{AWH2}, (CH₂)₀₋₆—N(R^{AWH2})₂, (CH₂)₀₋₆—C(O)R^{AWH2}(CH₂)₀₋₆—C(O)OR^{AWH2}, (CH₂)₀₋₆—C(O)N(R^{AWH2})₂, (CH₂)₀₋₆-C₃₋₁₀ cycloalkyl, (CH₂)₀₋₆-aryl, (CH₂)₀₋₆-heterocycle, and (CH₂)₀₋₆-heteroaryl;
R⁶ is selected from the group consisting of H, halogen, CN, C₁₋₆ alkyl, C₂₋₆ alkenyl, C₂₋₆ alkynyl, C₁₋₆ haloalkyl, C₁₋₆ alkoxy, (CH₂)₀₋₆—R^{AWH2}, (CH₂)₀₋₆—OR^{AWH2}, (CH₂)₀₋₆—N(R^{AWH2})₂, (CH₂)₀₋₆—C(O)R^{AWH2} (CH₂)₀₋₆—C(O)OR^{AWH2}, (CH₂)₀₋₆C(O)N(R^{AWH2})₂, (CH₂)₀₋₆—C₃₋₁₀ cycloalkyl, (CH₂)₀₋₆-aryl, (CH₂)₀₋₆-heterocycle, and (CH₂)₀₋₆-heteroaryl; and
each R^{AWH2} is independently H, halogen, OH, C₁₋₆ alkyl, C₂₋₆ alkenyl, C₂₋₆ alkynyl, C₁₋₆ haloalkyl, C₂₋₆ haloalkenyl, C₂₋₆ haloalkynyl, C₁₋₆ alkoxy, C₂₋₆ alkenoxy, C₂₋₆ alkynoxy, C₁₋₆ haloalkoxy, C₂₋₆ haloalkenoxy, and C₂₋₆ alkynoxy.

One aspect of the present disclosure includes wherein Y is N or CR^{3c}.

One aspect of the present disclosure includes wherein Y combines with R^{3a} and R^{3b} to form:
cycloalkyl substituted with B(OH)₂;
aryl substituted with B(OH)₂;
heterocyclyl, either
containing a B atom in the ring or ring system,
ii) substituted with B(OH)₂, or
iii) substituted with an optionally substituted 4- to 6-membered ring containing a B atom, one or more additional heteroatoms selected from O, N, or S, and one or more degrees of unsaturation;
or
heteroaryl, either
containing a B atom in the ring or ring system, or
substituted with B(OH)₂, or
substituted with an optionally substituted 4- to 6-membered ring containing a B atom, one or
more additional heteroatoms selected from O, N, or S, and one or more degrees of unsaturation;
and
wherein each Y-ring or ring system is optionally substituted.

One aspect of the present disclosure includes wherein Y^{T} is selected from the group consisting of substituted or unsubstituted:

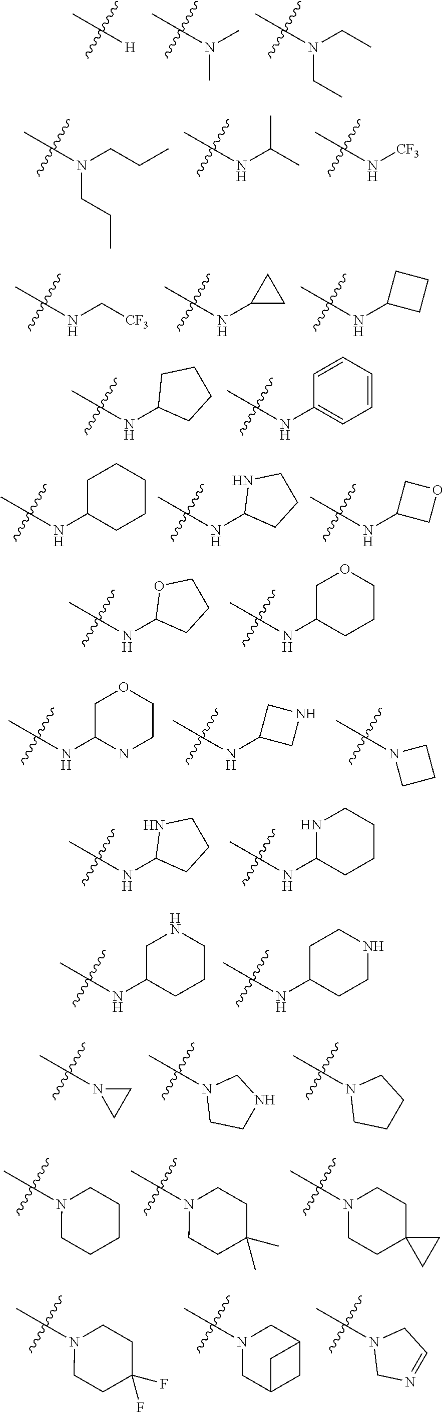

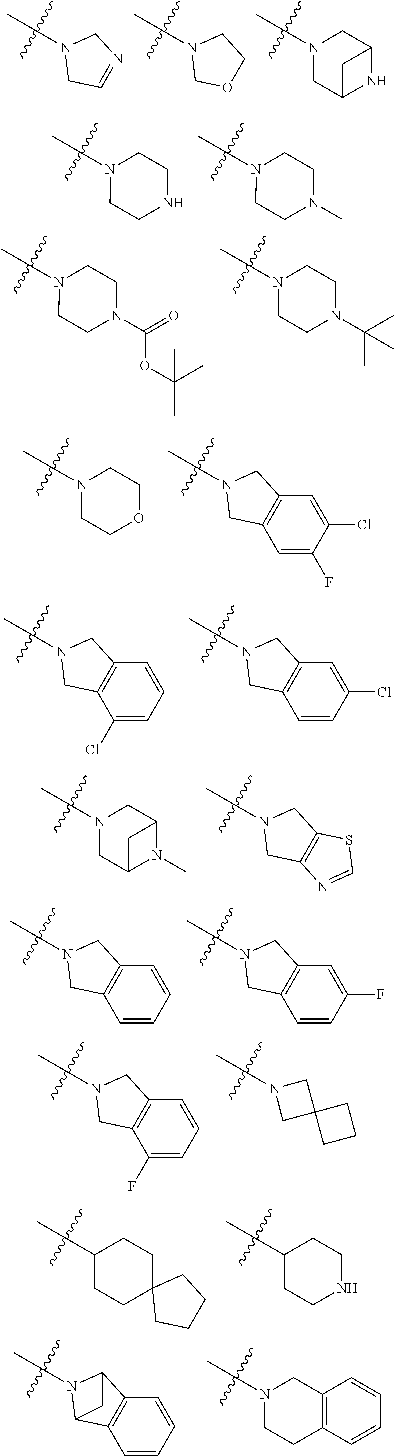

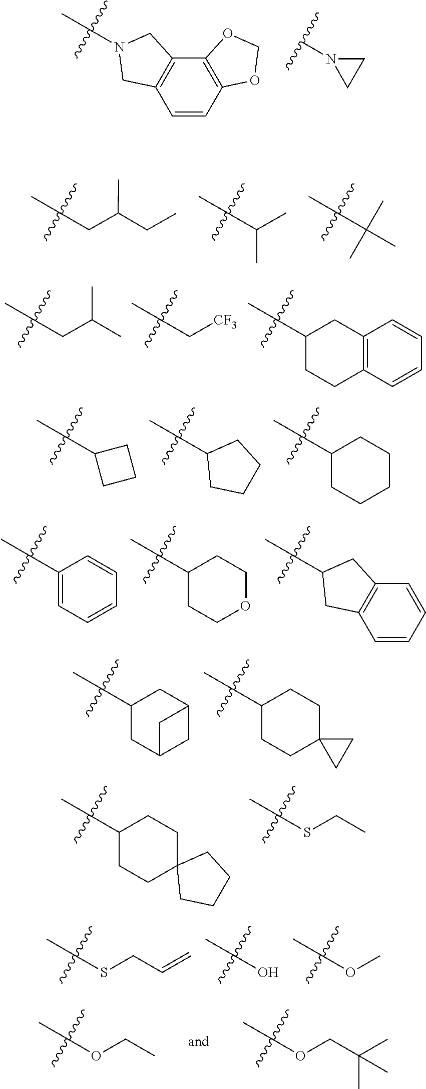

One aspect of the present disclosure includes wherein Y^{T} is selected from the group consisting of:

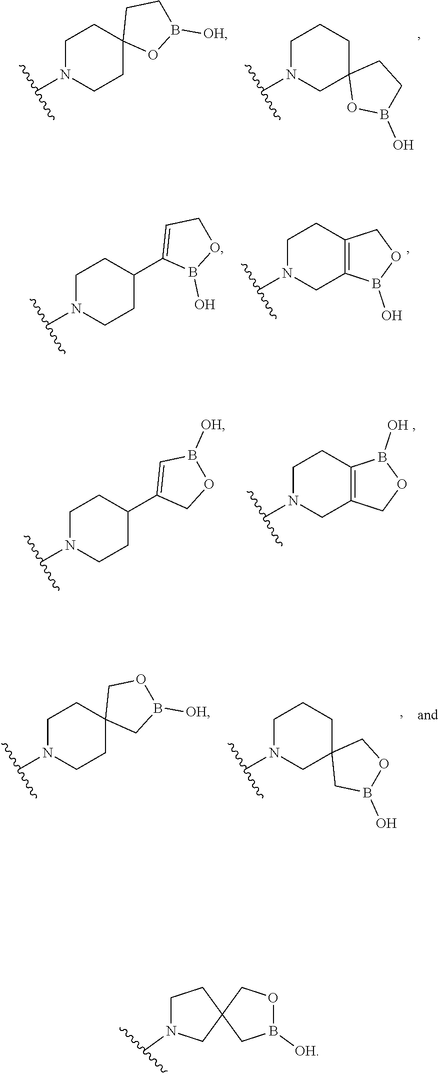

One aspect of the present disclosure includes wherein Y is CR^{3c}, R^{3c} is absent or H, and each of R^{3a} and R^{3b} is independently selected from the group consisting of H, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3- to 6-membered heterocyclyl.

As provided in more detail herein and as appreciated by those skilled in the art, embodiments of the present invention may be defined in terms of a warhead, which is intended to describe a functional group that offers chemical capture of a targeted amino acid residue on a target protein, including formation of covalent or hydrogen bonds with one or more targeted amino acids. As one aspect of warhead interaction, the burial of hydrophobic amino acids in the protein core is a driving force in protein folding. The extent to which an amino acid interacts with the solvent and the protein core is naturally proportional to the surface area exposed to the solvent environment. The accessible surface area (ASA) or solvent-accessible surface area (SASA) is the surface area of a biomolecule that is accessible to a solvent. The SASA is the surface characterized around a protein by a hypothetical center of a solvent sphere. As provided in more detail herein and as appreciated by those skilled in the art, the present disclosure references chemical capture, namely chemical bonds or other interactions that allow atoms to be more stable by, for example, by filling their valence shell of electrons. Chemical capture, and therefore warhead interaction with a targeted amino acid, includes covalent bonds, hydrogen bonds, ionic bonds, and van der Waals interactions.

One aspect of the present disclosure includes wherein WH provides chemical capture of a solvent accessible surface area of a protein.

One aspect of the present disclosure includes wherein the chemical capture is a binding interaction.

One aspect of the present disclosure includes wherein the binding interaction is covalent binding.

One aspect of the present disclosure includes wherein the binding interaction is hydrogen binding.

One aspect of the present disclosure includes wherein the solvent accessible surface area of a protein is an accessible amino acid.

One aspect of the present disclosure includes wherein the amino acid is arginine, histidine, lysine, glutamic acid, serine, threonine, or glutamine.

One aspect of the present disclosure includes wherein the amino acid is histidine.

One aspect of the present disclosure includes wherein WH is a boron-containing warhead.

One aspect of the present disclosure includes wherein WH is a non-boron-containing warhead.

One embodiment of the present disclosure includes a compound of Formula (AX):

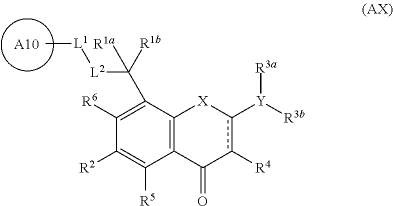

or a tautomer, enantiomer, diastereomer, mixture, salt, hydrate, solvate, or deuterated form thereof, wherein
A10 is a 5- to 14-membered mono- or fused-ring system comprising one or more of cycloalkyl, heterocyclyl, aryl, and heteroaryl,
each of heterocyclyl or heteroaryl rings may contain one or more heteroatoms selected from N, O, S, and B;
A10 is optionally substituted with one or more R^{A10};
each R^{A10} independently is selected from halogen, OH, C₁₋₆ alkyl, C₂₋₆ alkenyl, C₂₋₆ alkynyl, C₁₋₆ haloalkyl, C₂₋₆ haloalkenyl, C₂₋₆ haloalkynyl, oxo, (CH₂)₀₋₆CN, (CH₂)₁₋₆OH, (CH₂)₀₋₆O—C₁₋₆ alkyl, (CH₂)₀₋₆O—C₂₋₆ alkenyl, (CH₂)₀₋₆O—C₂₋₆ alkynyl, (CH₂)₀₋₆C(O)H, (CH₂)₀₋₆C(O)(C₁₋₆ alkyl, C₂₋₆ alkenyl, or C₁₋₆ alkynyl), (CH₂)₀₋₆C(O)OH, (CH₂)₀₋₆C(O)O(C₁₋₆ alkyl, C₂₋₆ alkenyl, or C₂₋₆ alkynyl), (CH₂)₀₋₆—NH₂, (CH₂)₀₋₆NH(C₁₋₆ alkyl, C₂₋₆ alkenyl, or C₂₋₆ alkynyl), (CH₂)₀₋₆—N(C₁₋₆ alkyl, C₂₋₆ alkenyl, or C₂₋₆ alkynyl)₂, (CH₂)₀₋₆C(O)NH₂, (CH₂)₀₋₆C(O)NH(C₁₋₆ alkyl, C₂₋₆ alkenyl, or C₂₋₆ alkynyl), (CH₂)₀₋₆C(O)N(C₁₋₆ alkyl, C₂₋₆ alkenyl, or C₂₋₆ alkynyl)₂, (CH₂)₀₋₆—NHC(O)H, (CH₂)₀₋₆—NHC(O)(C₁₋₆ alkyl, C₂₋₆ alkenyl, or C₂₋₆ alkynyl), (CH₂)₀₋₆—N(C₁₋₆ alkyl, C₂₋₆ alkenyl, and C₂₋₆ alkynyl)C(O)(C₁₋₆ alkyl, C₂₋₆ alkenyl, or C₂₋₆ alkynyl), B(OH)₂, SO₂-halogen, and O—SO₂-halogen;
L¹ is selected from the group consisting of a direct bond, optionally substituted arylene, and optionally substituted heteroarylene;
L² is selected from the group consisting of O and NH;
each of R^{1a} and R^{1b} independently is selected from the group consisting of H, halogen, CN, C₁₋₆ alkyl, C₂₋₆ alkenyl, C₂₋₆ alkynyl, C₁₋₆ haloalkyl, and C₁₋₆ alkoxy;
or L²-C(R^{1a})(R^{1b}) combines to form NHC(O), C(O)NH, NHS(O)₂, S(O)₂NH, or C(NH)NH₂;
X is C(R^{x})₂, O, NR^{x}, or S;
each R^{x} independently is selected from the group consisting of H, C₁₋₆ alkyl, C₂₋₆ alkenyl, and C₂₋₆ alkynyl;
Y is H, CR^{3c}, N, O, or S;
when Y is H, each of R^{3a} and R^{3b} is absent;
when Y is N,
each of R^{3a} and R^{3b} independently is selected from the group consisting of H, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl; or
Y, as an N atom, combines with R^{3a} and R^{3b} to form a heterocyclic or heteroaromatic mono ring or spiro or fused ring system, which may contain one or more additional heteroatoms selected from the group consisting of N, O, and S, and wherein the ring or ring system is optionally substituted;
when Y is CR^{3c}
R^{3c} is absent or is selected from the group consisting of hydrogen, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl;
each of R^{3a} and R^{3b} independently is selected from the group consisting of H, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl; or
Y, as an C atom, combines with R^{3a} and R^{3b} to form a cycloalkyl, aryl, heterocyclic, or heteroaromatic mono ring or spiro or fused ring system, where the heterocyclic and heteroaromatic rings contain one or more heteroatoms selected from the group consisting of N, O, and S, and wherein the Y-containing ring or ring system is optionally substituted; or
when Y is 0 or S, R^{3a} is absent and R^{3b} is selected from the group consisting of H, C₁₋₆ alkyl, C₂₋₆ alkenyl, and C₂₋₆ alkynyl;
R² is selected from the group consisting of H, halogen, CN, C₁₋₆ alkyl, C₂₋₆ alkenyl, C₂₋₆ alkynyl, C₁₋₆ haloalkyl, C₂₋₆ haloalkenyl, C₂₋₆ haloalkynyl, C₁₋₆ alkoxy, (CH₂)₀₋₆—R^{AX}, (CH₂)₀₋₆—OR^{AX}, (CH₂)₀₋₆—N(R^{AX})₂, (CH₂)₀₋₆—C(O)R^{AX}, (CH₂)₀₋₆—C(O)OR^{AX}, (CH₂)₀₋₆—C(O)N(R^{AX})₂, (CH₂)₀₋₆-C₃₋₁₀ cycloalkyl, (CH₂)₀₋₆-aryl, (CH₂)₀₋₆-heterocycle, and (CH₂)₀₋₆-heteroaryl;
R⁴ is selected from the group consisting of H, halogen, CN, C₁₋₆ alkyl, C₂₋₆ alkenyl, C₂₋₆ alkynyl, C₁₋₆ haloalkyl, C₂₋₆ haloalkenyl, C₂₋₆ haloalkynyl, C₁₋₆ alkoxy, (CH₂)₀₋₆—R, (CH₂)₀₋₆—OR^{AX}, (CH₂)₀₋₆—N(R^{AX})₂, (CH₂)₀₋₆—C(O)R^{AX}, (CH₂)₀₋₆—C(O)OR^{AX}, (CH₂)₀₋₆—C(O)N(R^{AX})₂, (CH₂)₀₋₆—C₃₋₁₀ cycloalkyl, (CH₂)₀₋₆-aryl, (CH₂)₀₋₆-heterocycle, and (CH₂)₀₋₆-heteroaryl;
R⁵ is selected from the group consisting of H, halogen, CN, C₁₋₆ alkyl, C₂₋₆ alkenyl, C₂₋₆ alkynyl, C₁₋₆ haloalkyl, C₂₋₆ haloalkenyl, C₂₋₆ haloalkynyl, C₁₋₆ alkoxy, (CH₂)₀₋₆—R^{AX}, (CH₂)₀₋₆—OR^{AX}, (CH₂)₀₋₆—N(R^{AX})₂, (CH₂)₀₋₆—C(O)R^{AX}, (CH₂)₀₋₆—C(O)OR^{AX}, (CH₂)₀₋₆—C(O)N(R^{AX})₂, (CH₂)₀₋₆-C₃₋₁₀ cycloalkyl, (CH₂)₀₋₆-aryl, (CH₂)₀₋₆-heterocycle, and (CH₂)₀₋₆-heteroaryl;
R⁶ is selected from the group consisting of H, halogen, CN, C₁₋₆ alkyl, C₂₋₆ alkenyl, C₂₋₆ alkynyl, C₁₋₆ haloalkyl, C₂₋₆ haloalkenyl, C₂₋₆ haloalkynyl, C₁₋₆ alkoxy, (CH₂)₀₋₆—R^{AX}, (CH₂)₀₋₆—OR^{AX}, (CH₂)₀₋₆—N(R^{AX})₂, (CH₂)₀₋₆—C(O)R^{AX}, (CH₂)₀₋₆—C(O)OR^{AX}, (CH₂)₀₋₆—C(O)N(R^{AX})₂, (CH₂)₀₋₆-C₃₋₁₀ cycloalkyl, (CH₂)₀₋₆-aryl, (CH₂)₀₋₆-heterocycle, and (CH₂)₀₋₆-heteroaryl; and
each R^{AX} is independently H, halogen, OH, C₁₋₆ alkyl, C₂₋₆ alkenyl, C₂₋₆ alkynyl, C₁₋₆ haloalkyl, C₂₋₆ haloalkenyl, C₂₋₆ haloalkynyl, C₁₋₆ alkoxy, C₂₋₆ alkenoxy, C₂₋₆ alkynoxy, C₁₋₆ haloalkoxy, C₂₋₆ haloalkenoxy, and C₂₋₆ alkynoxy.

One aspect of the present disclosure includes wherein at least one of A10 or Y(R^{3a})(R^{3b}) has one or more of: (a) a B atom in the defined ring or ring system; (b) is substituted with a B(OH)₂; or carries a ring or ring system substituent that contains a B atom in the ring or ring system.

One aspect of the present disclosure includes wherein A10 is selected from the group consisting of:

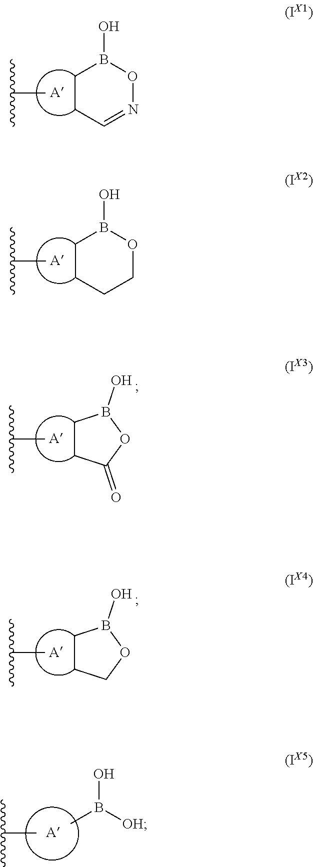

where each [symbol] represents the point of attachment to the depicted L¹;
each depicted A′ independently is a cycloalkyl, heterocyclyl, aryl, or heteroaryl; and each A10 is optionally substituted with one or more R^{A10} on either depicted ring.

One aspect of the present disclosure includes wherein A10 is:

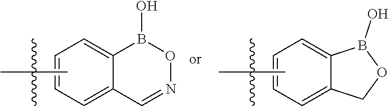

each optionally substituted with one or more R^{A10} on either depicted ring.

One aspect of the present disclosure includes wherein A10 is selected from the group consisting of:

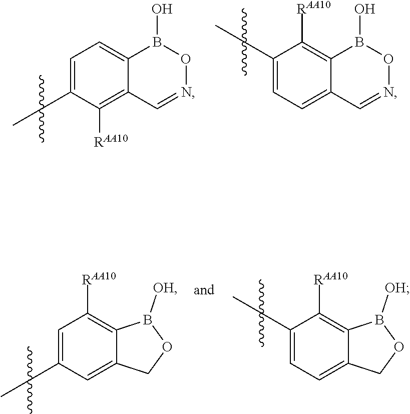

each R^{AA10} is independently selected from the group consisting of: halogen, C₁₋₆ alkyl, C₂₋₆ alkenyl, C₂₋₆ alkynyl, C₃-C₆ cycloalkyl, C₁₋₆ haloalkyl, C₂₋₆ haloalkenyl, C₂₋₆ haloalkynyl, OH, O—C₃-C₆ cycloalkyl, O—C₁₋₆ alkyl, O—C₂₋₆ alkenyl, O—C₂₋₆ alkynyl, and CN or a deuterated form thereof; and each A10 may be further substituted with one or more R^{A10}.

One aspect of the present disclosure includes wherein R^{AA10} is F, Cl, CH₃, OCH₃, or CDs.

One aspect of the present disclosure includes wherein A10 is not further substituted.

One aspect of the present disclosure includes wherein A10 is further substituted with one or more R^{A10}, each independently selected from the group consisting of: halogen, CN, C₁₋₆ alkyl, C₂₋₆ alkenyl, C₂₋₆ alkynyl, C₃-C₆ cycloalkyl, C₁₋₆ haloalkyl, C₂₋₆ haloalkenyl, C₂₋₆ haloalkynyl, OH, O—C₁₋₆ alkyl, 0-C₂₋₆ alkenyl, and O—C₂₋₆ alkynyl.

One aspect of the present disclosure includes wherein L¹ is:
substituted or unsubstituted arylene; or
substituted or unsubstituted heteroarylene.

The compound of any one of claims 69 to 77, wherein L¹ is
substituted or unsubstituted phenylene; or
substituted or unsubstituted 5- to 10-membered heteroarylene having one or more heteroatoms selected from N, O, and S.

One aspect of the present disclosure includes wherein L¹ is substituted or unsubstituted phenylene; or substituted or unsubstituted pyridiylene.

One aspect of the present disclosure includes wherein L¹ is substituted with one or more halogen, C₁-C₃ alkyl, C₁-C₃ haloalkyl, C₂-C₃ alkenyl, C₂-C₃ haloalkenyl, C₂-C₃ alkynyl, C₂-C₃ haloalkynyl, OH, O—(C₁₋₃ alkyl, C₂₋₃ alkenyl, C₂₋₃ alkynyl), NH₂, NH(C₁₋₃ alkyl, C₂₋₃ alkenyl, C₂₋₃ alkynyl), N(C₁₋₃ alkyl, C₂₋₃ alkenyl, C₂₋₃ alkynyl)₂, C₃ cycloalkyl, and C₃ halocycloalkyl.

One aspect of the present disclosure includes wherein L¹ is substituted with one or more halogen or C₁-C₃ alkyl.

One aspect of the present disclosure includes wherein X is oxygen.

One aspect of the present disclosure includes wherein Y is N or CR^{3c}.

One aspect of the present disclosure includes wherein Y is CR^{3c}; R³, is H and each of R^{3a} and R^{3b} independently is selected from the group consisting of H, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl.

One aspect of the present disclosure includes wherein Y combines with R^{3a} and R^{3b} to form:
cycloalkyl substituted with B(OH)₂;
aryl substituted with B(OH)₂;
heterocyclyl, either
containing a B atom in the ring or ring system,
ii) substituted with B(OH)₂, or
iii) substituted with an optionally substituted 4- to 6-membered ring containing a B atom, one or more additional heteroatoms selected from O, N, or S, and one or more degrees of unsaturation; or
heteroaryl, either
containing a B atom in the ring or ring system, or
substituted with B(OH)₂, or
substituted with an optionally substituted 4- to 6-membered ring containing a B atom, one or more additional heteroatoms selected from O, N, or S, and one or more degrees of unsaturation;
and
wherein each Y-ring or ring system is optionally substituted.

One aspect of the present disclosure includes wherein Y is nitrogen.

One aspect of the present disclosure includes wherein R^{1a} is CH₃.

One aspect of the present disclosure includes wherein R^{1b} is H.

One aspect of the present disclosure includes wherein each of R², R⁴, R⁵, and R⁶ independently is H, halogen, C₁₋₆ alkyl, C₂₋₆ alkenyl, C₂₋₆ alkynyl, C₃-C₆ cycloalkyl, C₂₋₆ haloalkenyl, C₂₋₆ haloalkynyl, or C₁₋₆ haloalkyl.

One aspect of the present disclosure includes wherein R² is CH₃.

One aspect of the present disclosure includes wherein R⁴ is H, C₁₋₆ alkyl, C₂₋₆ alkenyl, or C₂₋₆ alkynyl.

One aspect of the present disclosure includes wherein R⁴ is H or CH₃.

One aspect of the present disclosure includes wherein R⁵ is H.

One aspect of the present disclosure includes wherein R⁶ is H.

One aspect of the present disclosure includes wherein the depicted dashed bond is a double bond.

One embodiment of the present disclosure includes a compound of Formula Ia or Ib:

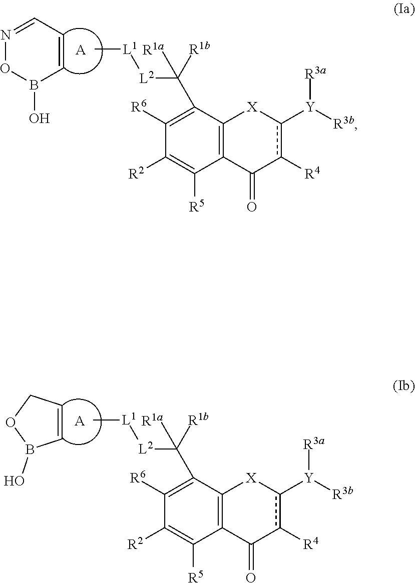

or a tautomer, enantiomer, diastereomer, mixture, salt, hydrate, solvate, or deuterated form thereof,
A is an aryl or heteroaryl ring, and together with the depicted boron ring, creates a fused ring system that includes the depicted boron-containing ring, and which system is optionally substituted with one or more R^{A};
each R^{A} independently is selected from halogen, OH, C₁₋₆ alkyl, C₂₋₆ alkenyl, C₂₋₆ alkynyl, C₁₋₆ haloalkyl, C₂₋₆ haloalkenyl, C₂₋₆ haloalkynyl, (CH₂)₀₋₆CN, (CH₂)₁₋₆OH, (CH₂)₀₋₆O—C₁₋₆ alkyl, (CH₂)₀₋₆O—C₂₋₆ alkenyl, (CH₂)₀₋₆O—C₂₋₆ alkynyl, (CH₂)₀₋₆C(O)H, (CH₂)₀₋₆C(O)(C₁₋₆ alkyl, C₂₋₆ alkenyl, or C₁₋₆ alkynyl), (CH₂)₀₋₆C(O)OH, (CH₂)₀₋₆C(O)O(C₁₋₆ alkyl, C₂₋₆ alkenyl, or C₂₋₆ alkynyl), (CH₂)₀₋₆—NH₂, (CH₂)₀₋₆NH(C₁₋₆ alkyl, C₂₋₆ alkenyl, or C₂₋₆ alkynyl), (CH₂)₀₋₆—N(C₁₋₆ alkyl, C₂₋₆ alkenyl, or C₂₋₆ alkynyl)₂, (CH₂)₀₋₆C(O)NH₂, (CH₂)₀₋₆C(O)NH(C₁₋₆ alkyl, C₂₋₆ alkenyl, or C₂₋₆ alkynyl), (CH₂)₀₋₆C(O)N(C₁₋₆ alkyl, C₂₋₆ alkenyl, or C₂₋₆ alkynyl)₂, (CH₂)₀₋₆—NHC(O)H, (CH₂)₀₋₆—NHC(O)(C₁₋₆ alkyl, C₂₋₆ alkenyl, or C₂₋₆ alkynyl), and (CH₂)₀₋₆₆N(C₁₋₆ alkyl, C₂₋₆ alkenyl, and C₂₋₆ alkynyl)C(O)(C₁₋₆ alkyl, C₂₋₆ alkenyl, or C₂₋₆ alkynyl) or a deuterated form thereof;
L¹ is selected from the group consisting of a direct bond, optionally substituted arylene, and optionally substituted heteroarylene;
L² is selected from the group consisting of O and NH;
each of R^{1a} and R^{1b} independently is selected from the group consisting of H, halogen, CN, C₁₋₆ alkyl, C₂₋₆ alkenyl, C₂₋₆ alkynyl, C₁₋₆ haloalkyl, and C₁₋₆ alkoxy;
X is C(R^{x})₂, O, NR^{x}, or S;
each R^{X} independently is selected from the group consisting of H, C₁₋₆ alkyl, C₂₋₆ alkenyl, and C₂₋₆ alkynyl;
each of R², R⁴, R⁵, and R⁶ independently is H, halogen, C₁₋₆ alkyl, C₂₋₆ alkenyl, C₂₋₆ alkynyl, C₃-C₆ cycloalkyl, C₂₋₆ haloalkenyl, C₂₋₆ haloalkynyl, or C₁₋₆ haloalkyl; and
Y is N or CR^{3a}; and
when Y is N,
each of R^{3a} and R^{3b} independently is selected from the group consisting of H, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl; or
R^{3a} and R^{3b} combine with the N atom to form a heterocyclic or heteroaromatic mono ring or spiro or fused ring system, which may contain one or more additional heteroatoms selected from the group consisting of N, O, and S, and wherein the ring or ring system is optionally substituted;
when Y is CR^{3c}
R^{3c} is absent or is selected from the group consisting of hydrogen, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl;
each of R^{3a} and R^{3b} independently is selected from the group consisting of H, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl; or
R^{3a} and R^{3b} combine with the C atom to form a cycloalkyl, aryl, heterocyclic, or heteroaromatic mono ring or spiro or fused ring system, where the heterocyclic and heteroaromatic rings contain one or more heteroatoms selected from the group consisting of N, O, and S, and wherein the Y-containing ring or ring system is optionally substituted.

In the event of any open valency in one or more compound of the present disclosure, such open valency may be considered to the hydrogen or an optional substitutent as herein defined.

One aspect of the present disclosure includes wherein A is a phenyl ring.

One aspect of the present disclosure includes wherein R^{A} is absent.

One aspect of the present disclosure includes wherein the fused ring system is substituted with one R^{A}.

One aspect of the present disclosure includes wherein the fused ring system is substituted with two R^{A}.

One aspect of the present disclosure includes wherein each R^{A} is independently selected from the group consisting of C₁₋₆ alkyl, O—C₁₋₆ alkyl, C₁₋₆ haloalkyl, and halogen, or a deuterated form thereof.

One aspect of the present disclosure includes wherein each R^{A} is independently selected from the group consisting of halogen, CD₃, CH₃, OCH₃, and CF₃.

One aspect of the present disclosure includes wherein L¹ is optionally substituted phenylene.

One aspect of the present disclosure includes wherein L¹ is phenylene substituted with one or more halogen or C₁₋₆alkyl.

One aspect of the present disclosure includes wherein L¹ is optionally substituted heteroarylene.

One aspect of the present disclosure includes wherein L¹ is optionally substituted pyridinylene.

One aspect of the present disclosure includes wherein L¹ is pyridinylene substituted with one or more halogen or C₁₋₆alkyl.

One aspect of the present disclosure includes wherein L¹ is substituted with one or two halogen.

One aspect of the present disclosure includes wherein the pyridinylene is attached as:

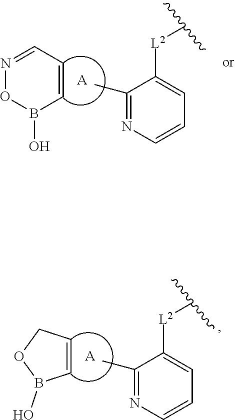

in either instance the depicted fused ring is optionally substituted with one or more R^{A}.

One aspect of the present disclosure includes wherein the depicted L¹ moiety is substituted para to the depicted B atom in the fused ring system:

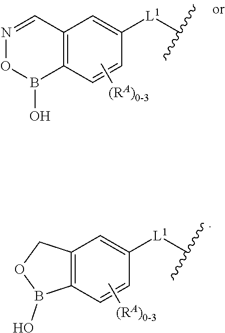

One aspect of the present disclosure includes wherein the depicted L¹ moiety is substituted meta to the depicted B atom in the fused ring system.

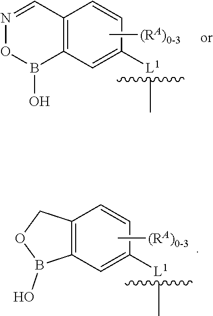

One aspect of the present disclosure includes wherein L² is NH.

One aspect of the present disclosure includes wherein R^{1a} is H; and R^{1b} independently is selected from the group consisting of H, halogen, CN, C₁₋₆ alkyl, C₂₋₆ alkenyl, C₂₋₆ alkynyl, C₁₋₆haloalkyl, and C₁₋₆alkoxy.

One aspect of the present disclosure includes wherein R^{1b} is C₁₋₆ alkyl.

One aspect of the present disclosure includes wherein R^{1b} is methyl.

One aspect of the present disclosure includes wherein the chiral center is in the R configuration, as depicted by:

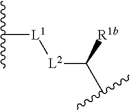

One aspect of the present disclosure includes wherein X is O.

One aspect of the present disclosure includes wherein
Y is CH and each of R^{3a} and R^{3b} is selected from the group consisting of C₁₋₆ alkyl, C₂₋₆ alkenyl, C₂₋₆alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl; or
Y is CH and combines with R^{3a} and R^{3b} to form a cycloalkyl or heterocyclic mono ring system comprising one heteroatom selected from N and 0.

One aspect of the present disclosure includes wherein Y is CH and each of R^{3a} and R^{3b} is C₁₋₃alkyl.

One aspect of the present disclosure includes wherein each of R^{3a} and R^{3b} is CH₃.

One aspect of the present disclosure includes wherein R² is CH₃.

One aspect of the present disclosure includes wherein R⁴ is H, C₁₋₆ alkyl, C₂₋₆ alkenyl, or C₂₋₆alkynyl.

One aspect of the present disclosure includes wherein R⁴ is H or CH₃.

One aspect of the present disclosure includes wherein R⁵ is H.

One aspect of the present disclosure includes wherein R⁶ is H.

The One aspect of the present disclosure includes wherein the depicted dashed bond is a double bond.

One embodiment of the present disclosure includes a compound of Formula Xa or Xb:

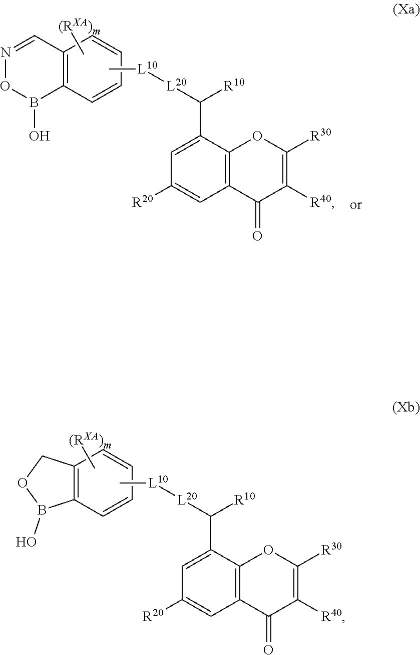

or a tautomer, enantiomer, diastereomer, mixture, salt, hydrate, solvate, or deuterated form thereof, wherein
each m is independently 0, 1, 2, 3, 4, or 5;.
each R^{XA} independently is selected from halogen, C₁₋₆ alkyl, C₂₋₆ alkenyl, C₂₋₆ alkynyl, C₁₋₆ haloalkyl, C₁₋₆ alkoxy, and C₃₋₆ cycloalkyl, or a deuterated form thereof;
L¹⁰ is selected from the group consisting of a direct bond, optionally substituted arylene, or optionally susbstituted heteroarylene;
L²⁰ is selected from the group consisting of O and NH;
R¹⁰ is selected from the group consisting of H and C₁₋₆ alkyl;
R²⁰ is selected from the group consisting of H, halogen, C₁-C₆ haloalkyl, and C₁₋₆ alkyl;
R³⁰ is selected from the group consisting of H or optionally substituted C₁₋₆ alkyl, C₂₋₆ alkenyl, C₂₋₆ alkynyl, C₃₋₆ cycloalkyl, N(C₁-C₆ alkyl), 6-membered aryl, 5- or 6-membered heteroaryl, or 3- to 10-membered heterocyclyl; and
R⁴⁰ is selected from the group consisting of H and C₁₋₆ alkyl.

One aspect of the present disclosure includes wherein L¹⁰ is an optionally substituted phenylene.

One aspect of the present disclosure includes wherein L¹⁰ is phenylene substituted with one or more halogen.

One aspect of the present disclosure includes wherein L¹⁰ is optionally substituted heteroarylene.

One aspect of the present disclosure includes wherein L¹⁰ is optionally substituted pyridinylene.

One aspect of the present disclosure includes wherein L¹⁰ is pyridinylene substituted with one or more halogen.

One aspect of the present disclosure includes wherein a compound, as optionally substituted:

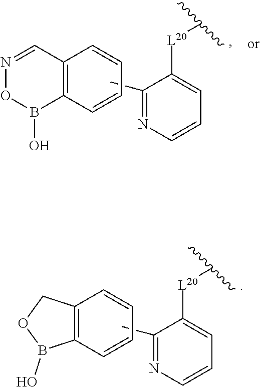

One aspect of the present disclosure includes wherein L²⁰ is NH.

One aspect of the present disclosure includes wherein R¹⁰ is C₁₋₆ alkyl.

One aspect of the present disclosure includes wherein R¹⁰ is CH₃.

One aspect of the present disclosure includes wherein R²⁰ is C₁₋₆ alkyl.

One aspect of the present disclosure includes wherein R²⁰ is CH₃.

One aspect of the present disclosure includes wherein R⁴⁰ is C₁₋₆ alkyl.

One aspect of the present disclosure includes wherein R⁴¹ is CH₃.

One aspect of the present disclosure includes wherein R³⁰ is optionally substituted aryl, heteroaryl, or heterocyclyl.

One aspect of the present disclosure includes wherein the optionally substituted heterocyclyl comprises 3- to 6-ring atoms, with 1 or 2 of the ring atoms being independently selected from the group consisting of nitrogen and oxygen.

One aspect of the present disclosure includes wherein the optionally substituted heterocyclyl is optionally substituted piperidinyl.

One aspect of the present disclosure includes wherein the optionally substituted piperidinyl is piperidinyl substituted with gem di-methyl.

One aspect of the present disclosure includes wherein the optionally substituted heterocyclyl is optionally substituted morpholinyl.

One aspect of the present disclosure includes wherein the optionally substituted heterocyclyl comprises three to six ring atoms, wherein one atom is oxygen.

One aspect of the present disclosure includes wherein R³⁰ is optionally substituted: C₁₋₆ alkyl, C₂₋₆ alkenyl, or C₂₋₆ alkynyl.

One aspect of the present disclosure includes wherein the compound is of formula XI:

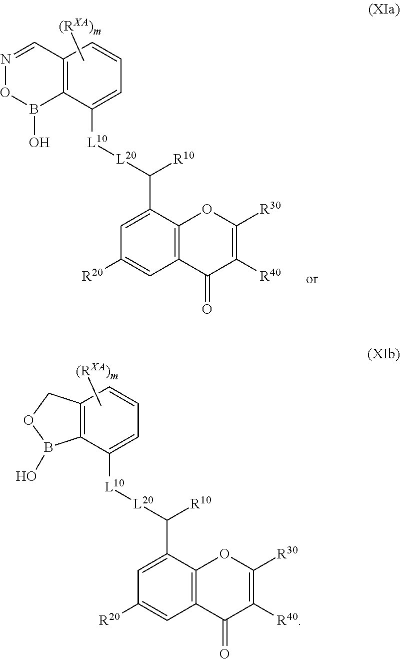

One aspect of the present disclosure includes wherein the compound is of formula XII:

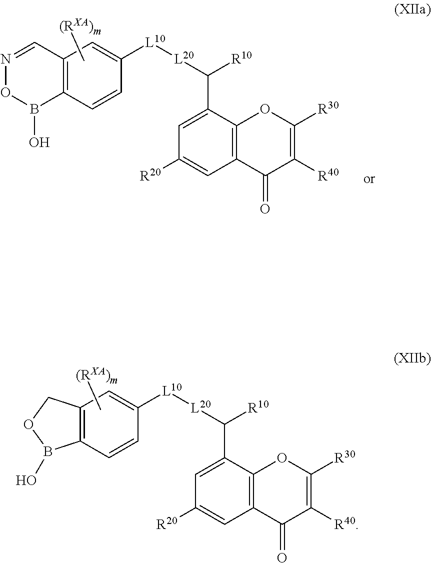

One aspect of the present disclosure includes wherein the compound is of formula XLII:

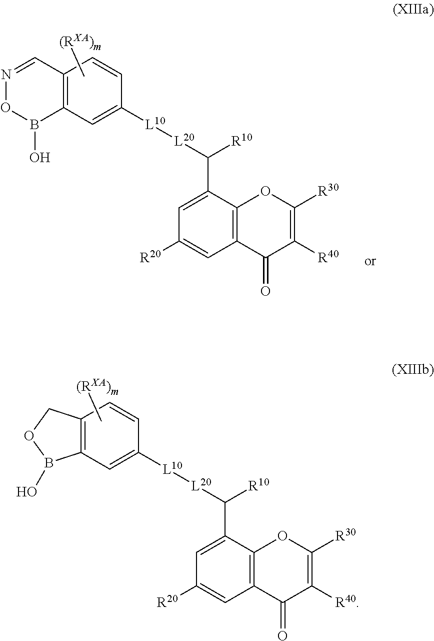

One embodiment of the present disclosure includes a compound selected from the group consisting of:

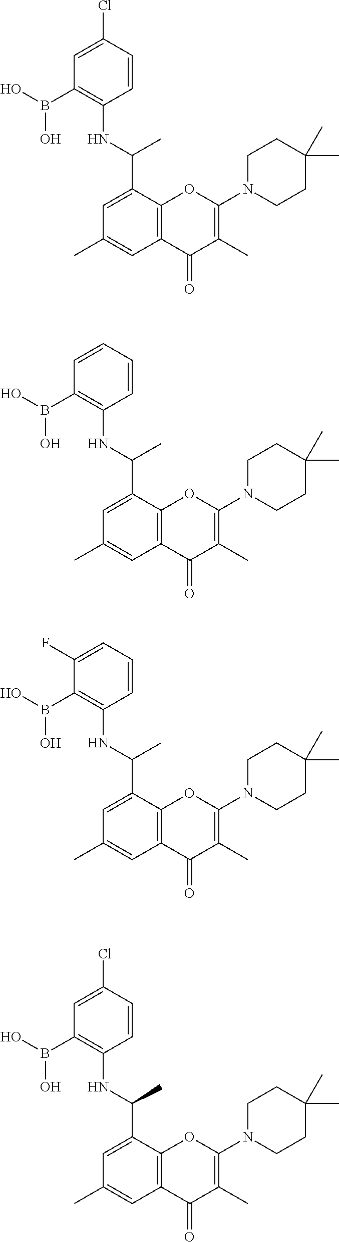 [SMILES: Cc1cc(C(C)Nc2ccc(Cl)cc2B(O)O)c2oc(N3CCC(C)(C)CC3)c(C)c(=O)c2c1 ; Cc1cc(C(C)Nc2ccccc2B(O)O)c2oc(N3CCC(C)(C)CC3)c(C)c(=O)c2c1 ; Cc1cc(C(C)Nc2cccc(F)c2B(O)O)c2oc(N3CCC(C)(C)CC3)c(C)c(=O)c2c1 ; Cc1cc([C@H](C)Nc2ccc(Cl)cc2B(O)O)c2oc(N3CCC(C)(C)CC3)c(C)c(=O)c2c1]

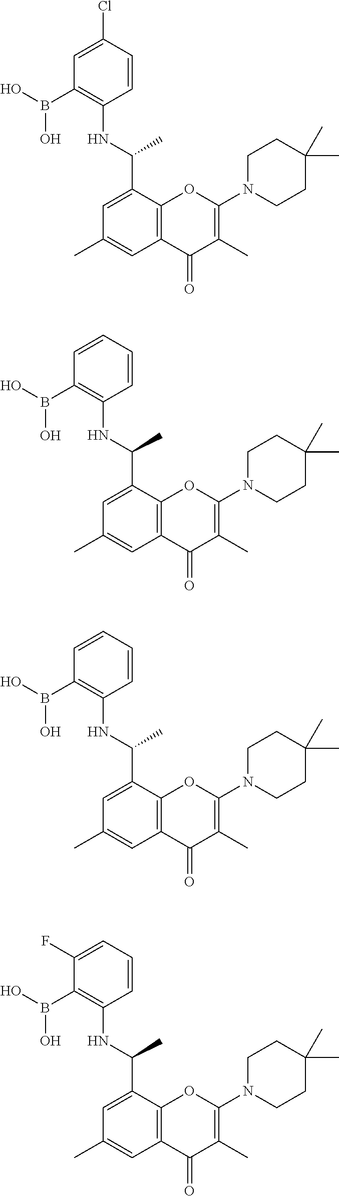 [SMILES: Cc1cc([C@@H](C)Nc2ccc(Cl)cc2B(O)O)c2oc(N3CCC(C)(C)CC3)c(C)c(=O)c2c1 ; Cc1cc([C@H](C)Nc2ccccc2B(O)O)c2oc(N3CCC(C)(C)CC3)c(C)c(=O)c2c1 ; Cc1cc([C@@H](C)Nc2ccccc2B(O)O)c2oc(N3CCC(C)(C)CC3)c(C)c(=O)c2c1 ; Cc1cc([C@H](C)Nc2cccc(F)c2B(O)O)c2oc(N3CCC(C)(C)CC3)c(C)c(=O)c2c1]

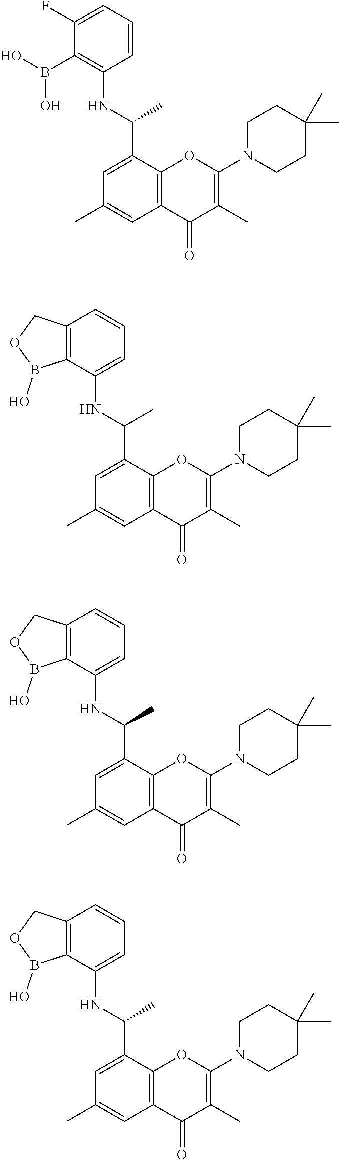 [SMILES: Cc1cc([C@@H](C)Nc2cccc(F)c2B(O)O)c2oc(N3CCC(C)(C)CC3)c(C)c(=O)c2c1 ; Cc1cc(C(C)Nc2cccc3c2B(O)OC3)c2oc(N3CCC(C)(C)CC3)c(C)c(=O)c2c1 ; Cc1cc([C@H](C)Nc2cccc3c2B(O)OC3)c2oc(N3CCC(C)(C)CC3)c(C)c(=O)c2c1 ; Cc1cc([C@@H](C)Nc2cccc3c2B(O)OC3)c2oc(N3CCC(C)(C)CC3)c(C)c(=O)c2c1]

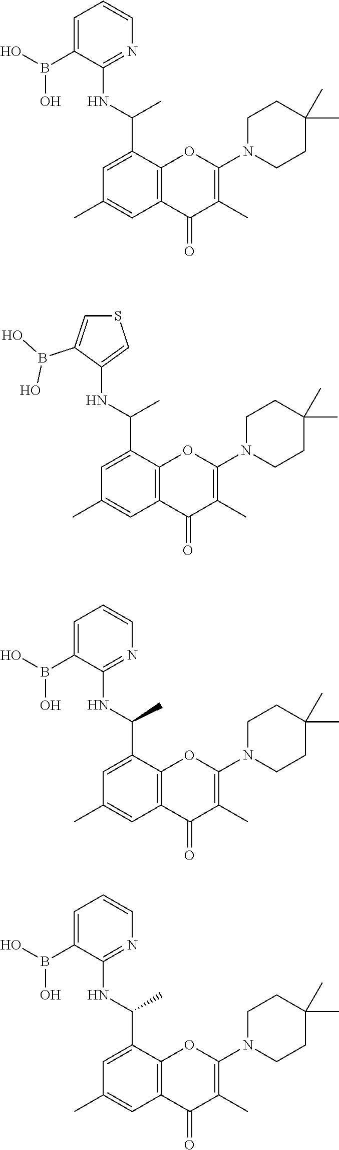 [SMILES: Cc1cc(C(C)Nc2ncccc2B(O)O)c2oc(N3CCC(C)(C)CC3)c(C)c(=O)c2c1 ; Cc1cc(C(C)Nc2cscc2B(O)O)c2oc(N3CCC(C)(C)CC3)c(C)c(=O)c2c1 ; Cc1cc([C@H](C)Nc2ncccc2B(O)O)c2oc(N3CCC(C)(C)CC3)c(C)c(=O)c2c1 ; Cc1cc([C@@H](C)Nc2ncccc2B(O)O)c2oc(N3CCC(C)(C)CC3)c(C)c(=O)c2c1]

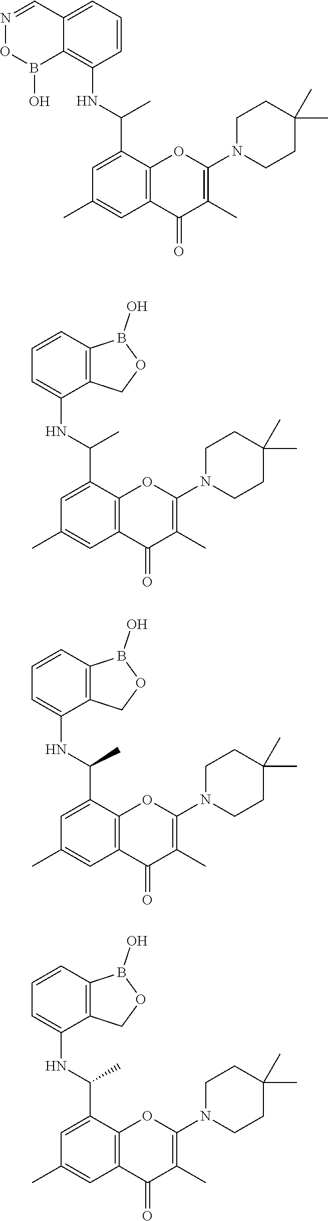

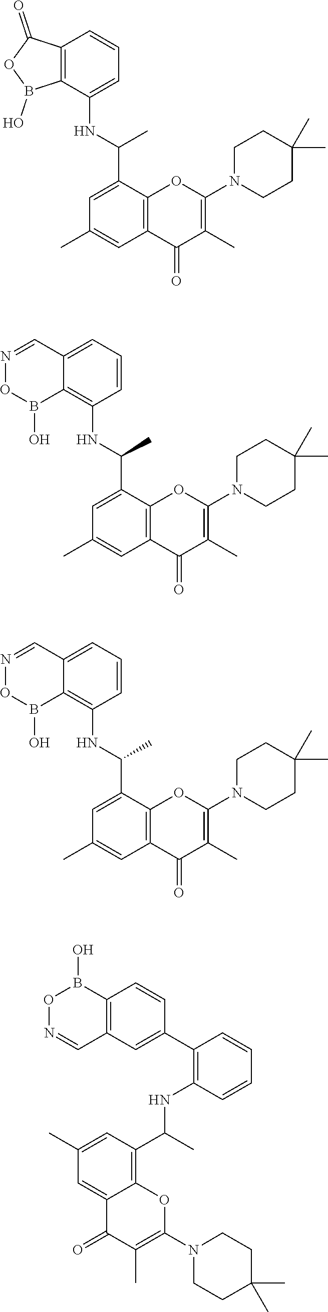 [SMILES: Cc1cc(C(C)Nc2cccc3c2B(O)OC3=O)c2oc(N3CCC(C)(C)CC3)c(C)c(=O)c2c1 ; Cc1cc([C@H](C)Nc2cccc3c2B(O)ON=C3)c2oc(N3CCC(C)(C)CC3)c(C)c(=O)c2c1 ; Cc1cc([C@@H](C)Nc2cccc3c2B(O)ON=C3)c2oc(N3CCC(C)(C)CC3)c(C)c(=O)c2c1 ; Cc1cc(C(C)Nc2ccccc2-c2ccc3c(c2)C=NOB3O)c2oc(N3CCC(C)(C)CC3)c(C)c(=O)c2c1]

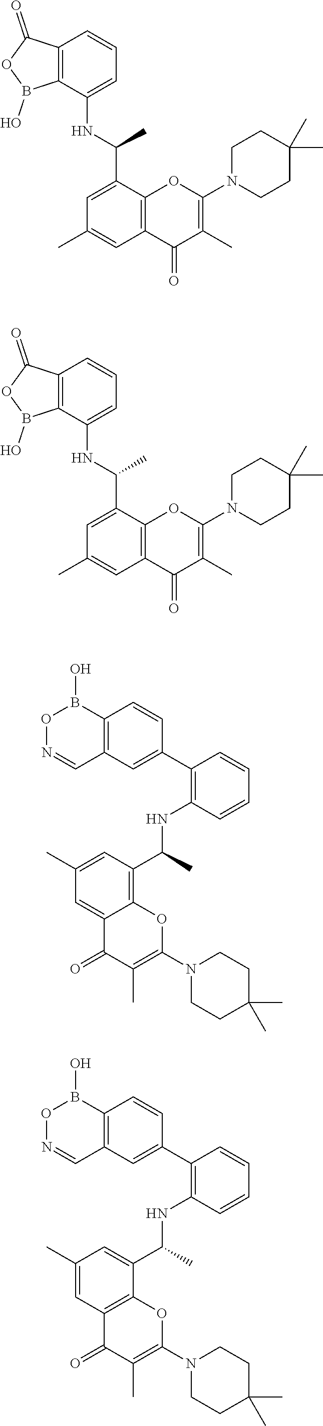 [SMILES: Cc1cc([C@H](C)Nc2cccc3c2B(O)OC3=O)c2oc(N3CCC(C)(C)CC3)c(C)c(=O)c2c1 ; Cc1cc([C@@H](C)Nc2cccc3c2B(O)OC3=O)c2oc(N3CCC(C)(C)CC3)c(C)c(=O)c2c1 ; Cc1cc([C@H](C)Nc2ccccc2-c2ccc3c(c2)C=NOB3O)c2oc(N3CCC(C)(C)CC3)c(C)c(=O)c2c1 ; Cc1cc([C@@H](C)Nc2ccccc2-c2ccc3c(c2)C=NOB3O)c2oc(N3CCC(C)(C)CC3)c(C)c(=O)c2c1]

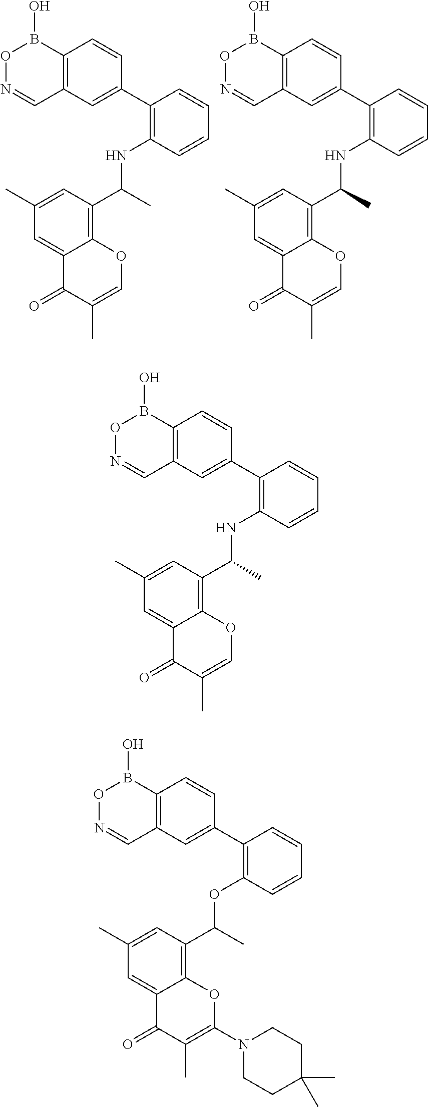

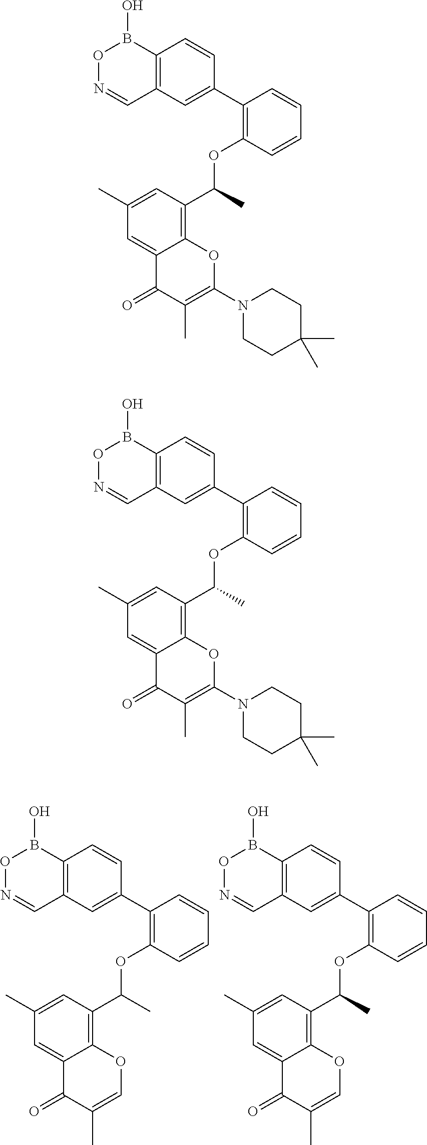

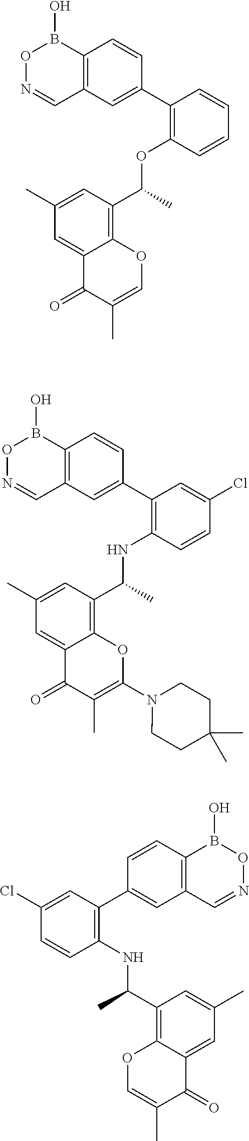 [SMILES: Cc1cc([C@@H](C)Oc2ccccc2-c2ccc3c(c2)C=NOB3O)c2occ(C)c(=O)c2c1 ; Cc1cc([C@@H](C)Nc2ccc(Cl)cc2-c2ccc3c(c2)C=NOB3O)c2oc(N3CCC(C)(C)CC3)c(C)c(=O)c2c1 ; Cc1cc([C@@H](C)Nc2ccc(Cl)cc2-c2ccc3c(c2)C=NOB3O)c2occ(C)c(=O)c2c1]

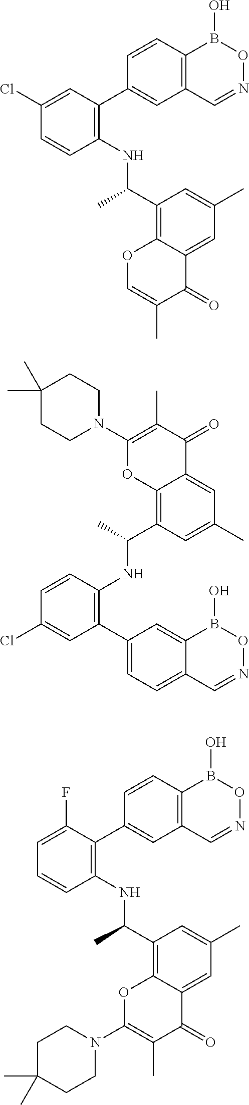

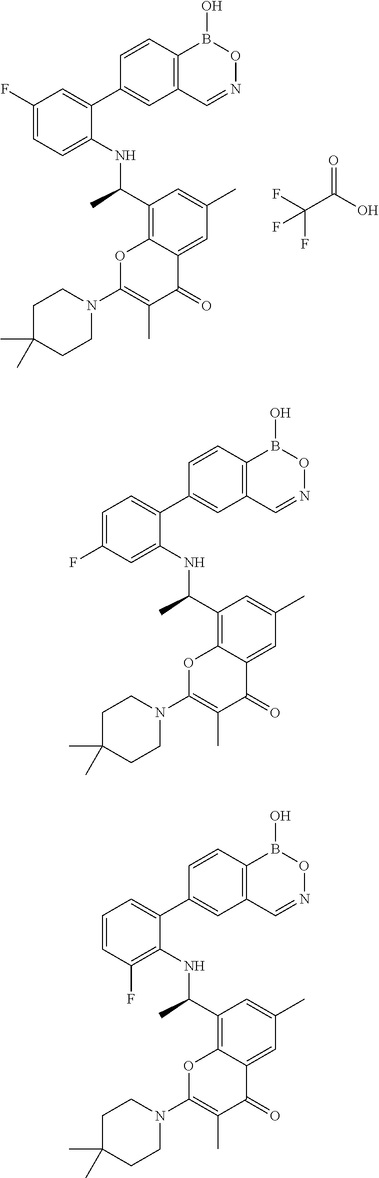

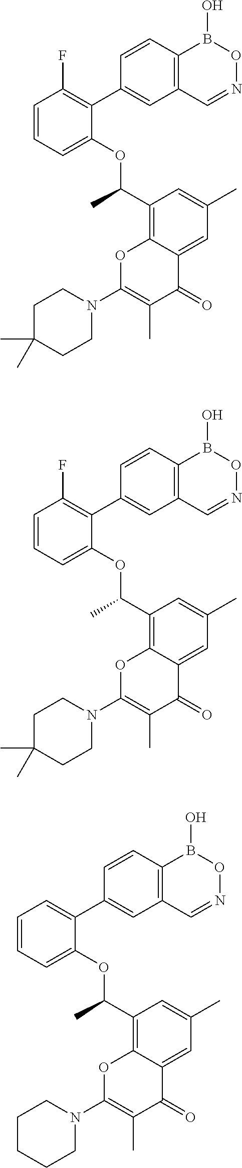

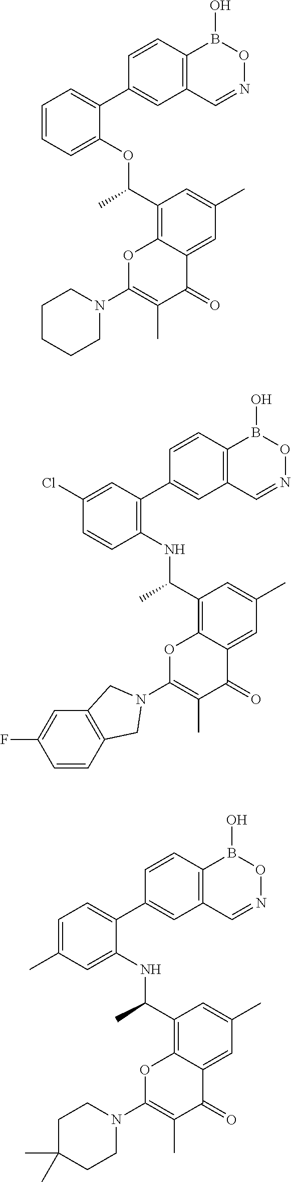

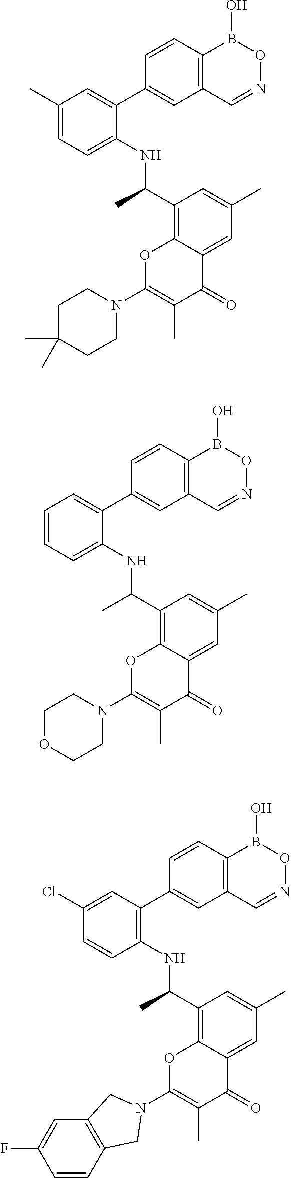

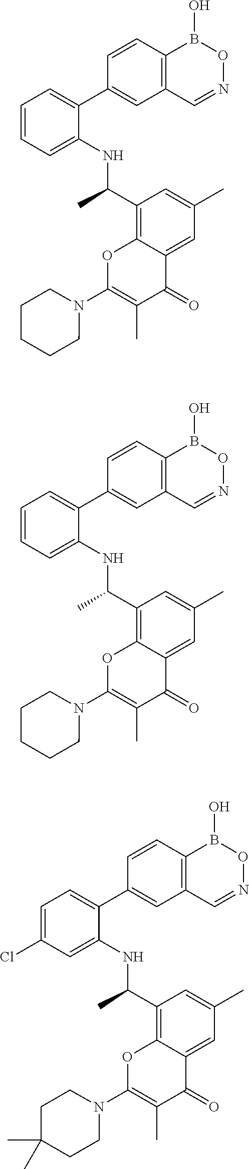

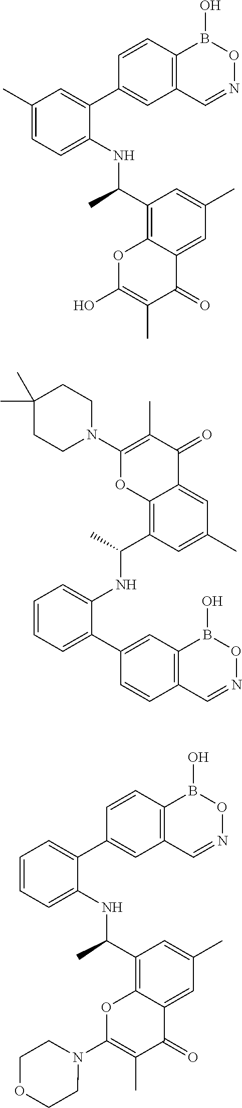

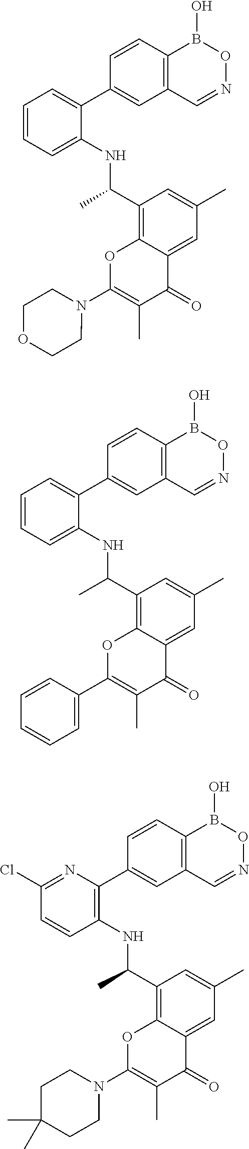

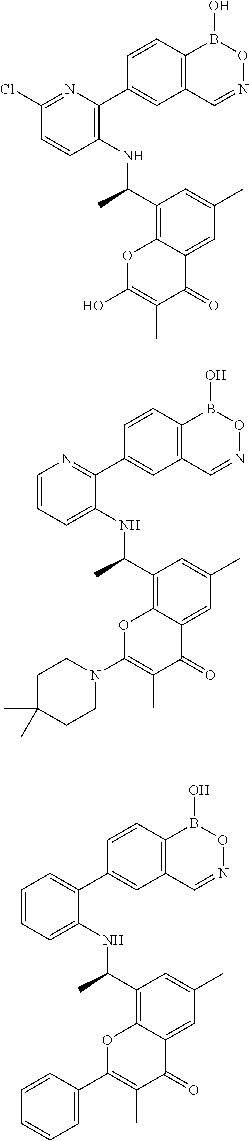

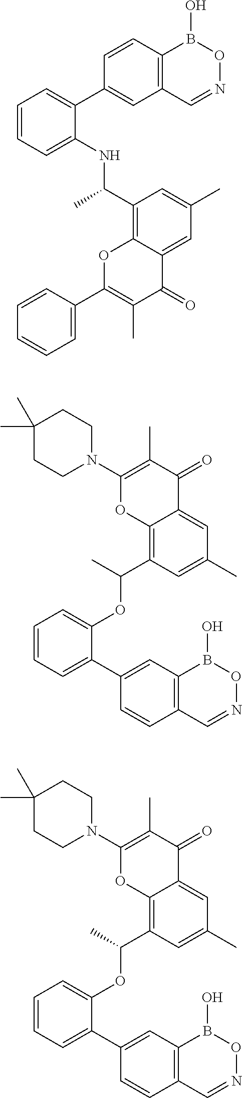

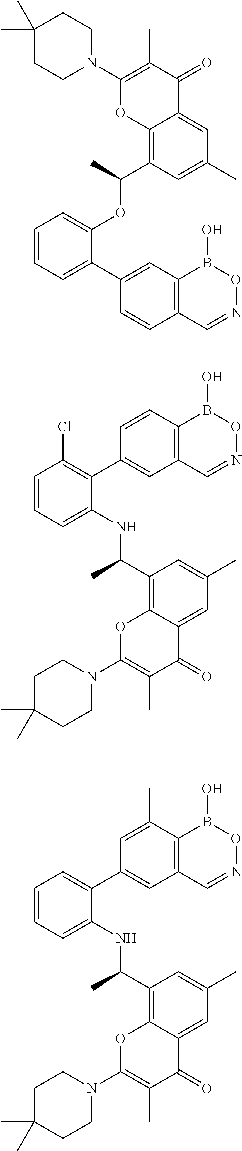

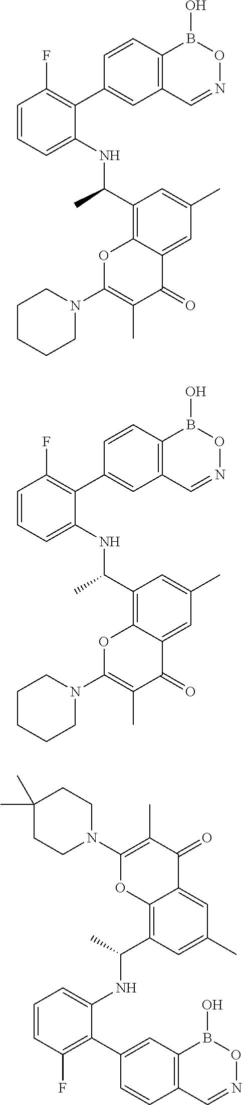

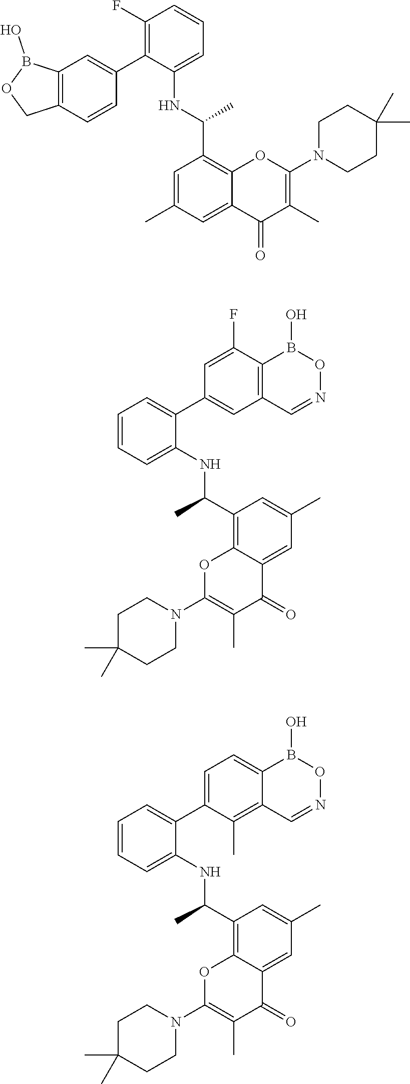

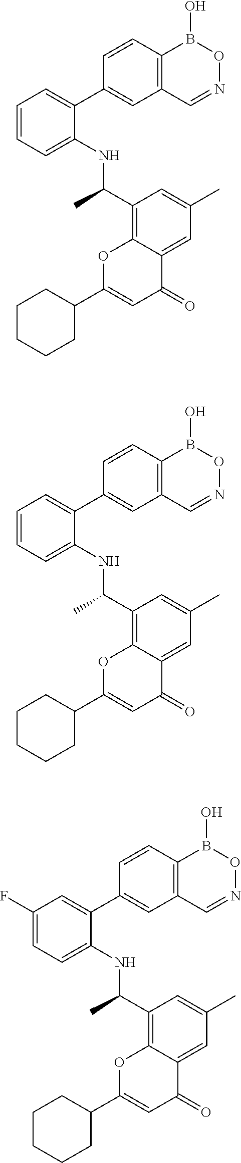 [SMILES: Cc1cc([C@@H](C)Nc2ccccc2-c2ccc3c(c2)C=NOB3O)c2oc(C3CCCCC3)cc(=O)c2c1 ; Cc1cc([C@H](C)Nc2ccccc2-c2ccc3c(c2)C=NOB3O)c2oc(C3CCCCC3)cc(=O)c2c1 ; Cc1cc([C@@H](C)Nc2ccc(F)cc2-c2ccc3c(c2)C=NOB3O)c2oc(C3CCCCC3)cc(=O)c2c1]

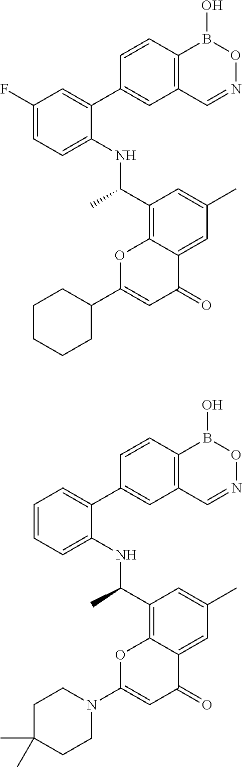

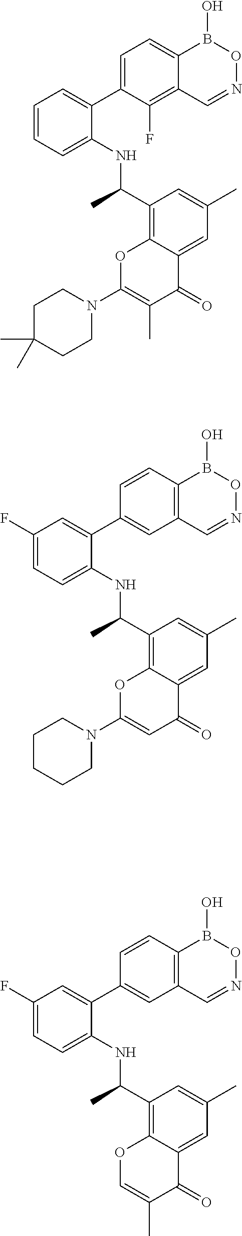 [SMILES: Cc1cc([C@@H](C)Nc2ccccc2-c2ccc3c(c2F)C=NOB3O)c2oc(N3CCC(C)(C)CC3)c(C)c(=O)c2c1 ; Cc1cc([C@@H](C)Nc2ccc(F)cc2-c2ccc3c(c2)C=NOB3O)c2oc(N3CCCCC3)cc(=O)c2c1 ; Cc1cc([C@@H](C)Nc2ccc(F)cc2-c2ccc3c(c2)C=NOB3O)c2occ(C)c(=O)c2c1]

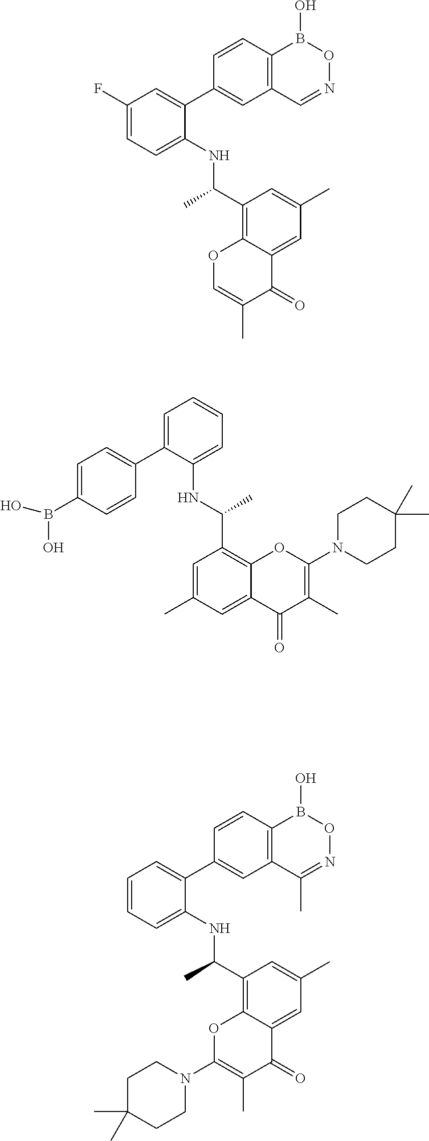

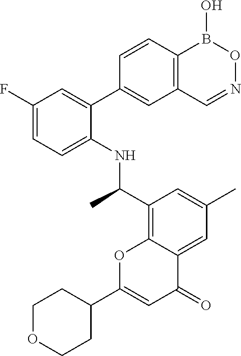 [SMILES: Cc1cc([C@@H](C)Nc2ccc(F)cc2-c2ccc3c(c2)C=NOB3O)c2oc(C3CCOCC3)cc(=O)c2c1]

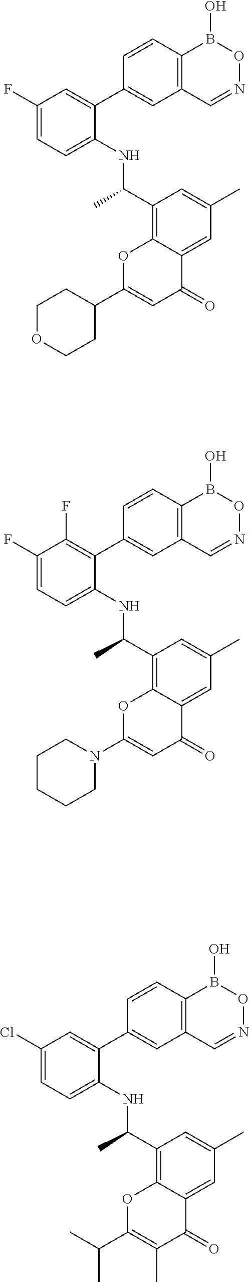 [SMILES: Cc1cc([C@H](C)Nc2ccc(F)cc2-c2ccc3c(c2)C=NOB3O)c2oc(C3CCOCC3)cc(=O)c2c1 ; Cc1cc([C@@H](C)Nc2ccc(F)c(F)c2-c2ccc3c(c2)C=NOB3O)c2oc(N3CCCCC3)cc(=O)c2c1 ; Cc1cc([C@@H](C)Nc2ccc(Cl)cc2-c2ccc3c(c2)C=NOB3O)c2oc(C(C)C)c(C)c(=O)c2c1]

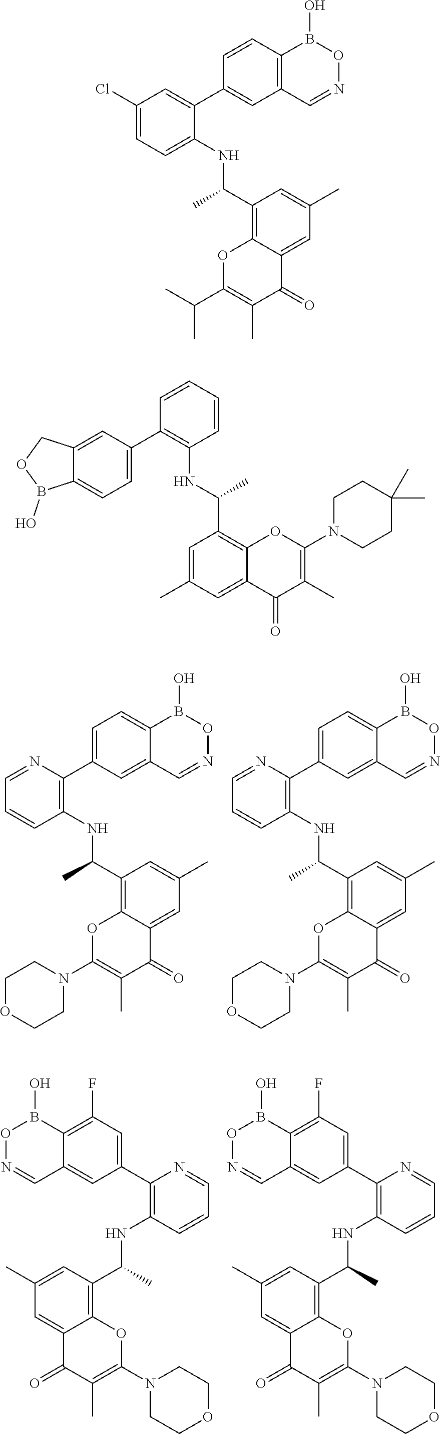

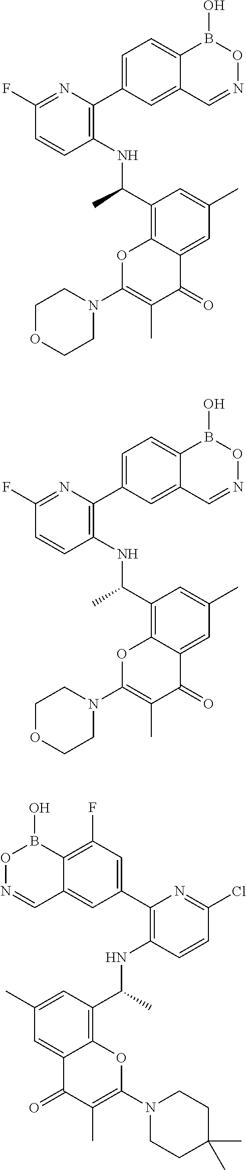

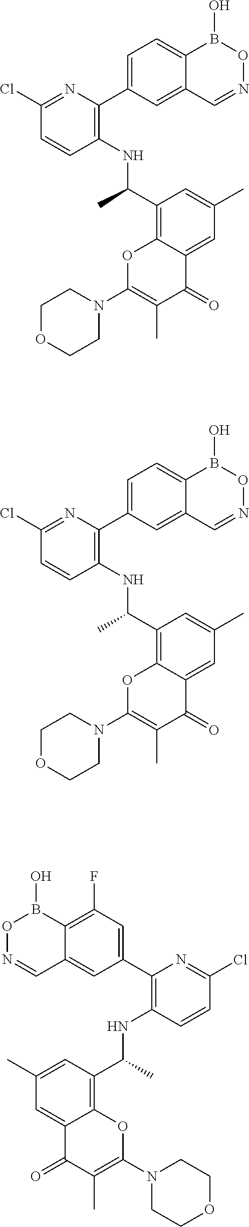 [SMILES: Cc1cc([C@@H](C)Nc2ccc(Cl)nc2-c2ccc3c(c2)C=NOB3O)c2oc(N3CCOCC3)c(C)c(=O)c2c1 ; Cc1cc([C@H](C)Nc2ccc(Cl)nc2-c2ccc3c(c2)C=NOB3O)c2oc(N3CCOCC3)c(C)c(=O)c2c1 ; Cc1cc([C@@H](C)Nc2ccc(Cl)nc2-c2cc(F)c3c(c2)C=NOB3O)c2oc(N3CCOCC3)c(C)c(=O)c2c1]

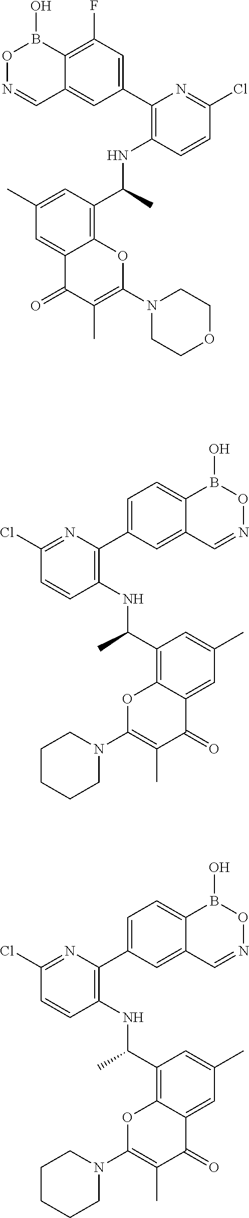 [SMILES: Cc1cc([C@H](C)Nc2ccc(Cl)nc2-c2cc(F)c3c(c2)C=NOB3O)c2oc(N3CCOCC3)c(C)c(=O)c2c1 ; Cc1cc([C@@H](C)Nc2ccc(Cl)nc2-c2ccc3c(c2)C=NOB3O)c2oc(N3CCCCC3)c(C)c(=O)c2c1 ; Cc1cc([C@H](C)Nc2ccc(Cl)nc2-c2ccc3c(c2)C=NOB3O)c2oc(N3CCCCC3)c(C)c(=O)c2c1]

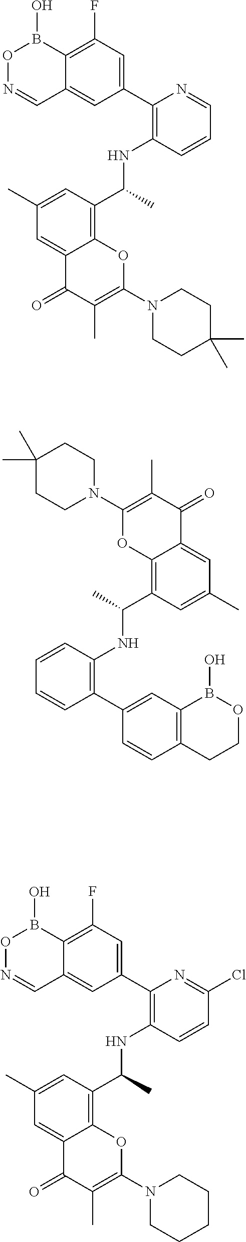

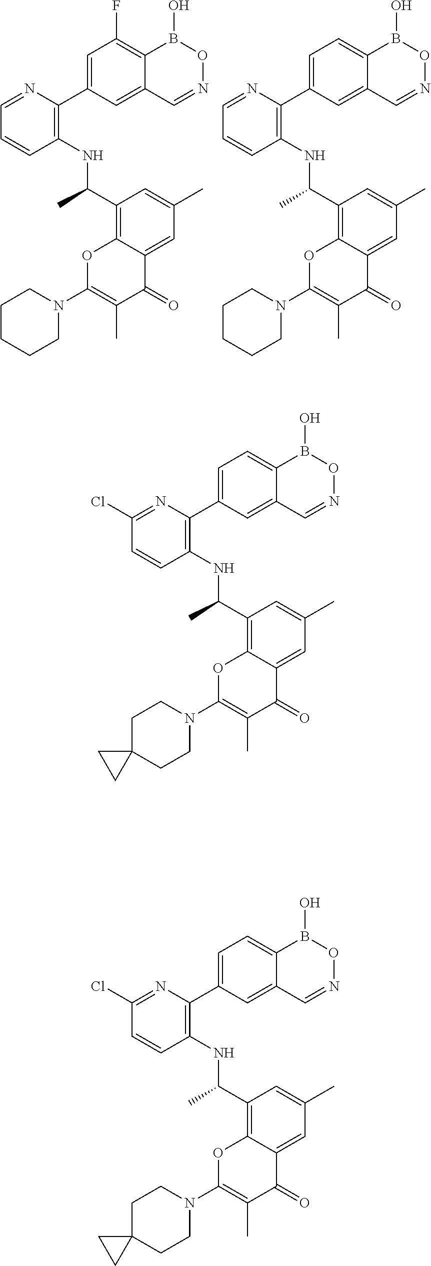

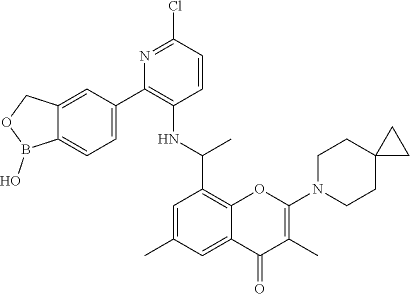 [SMILES: Cc1cc(C(C)Nc2ccc(Cl)nc2-c2ccc3c(c2)COB3O)c2oc(N3CCC4(CC3)CC4)c(C)c(=O)c2c1]

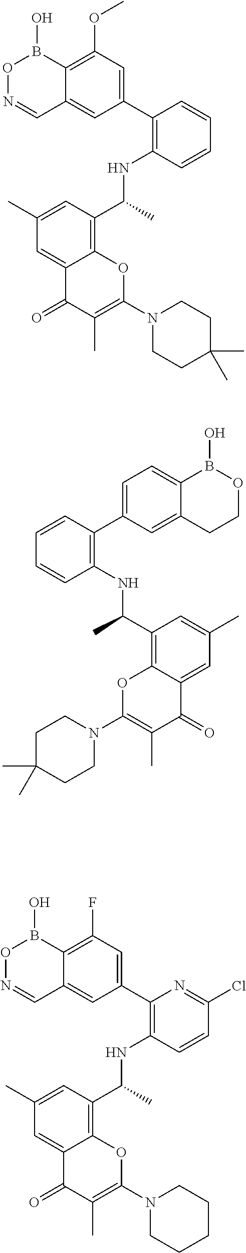

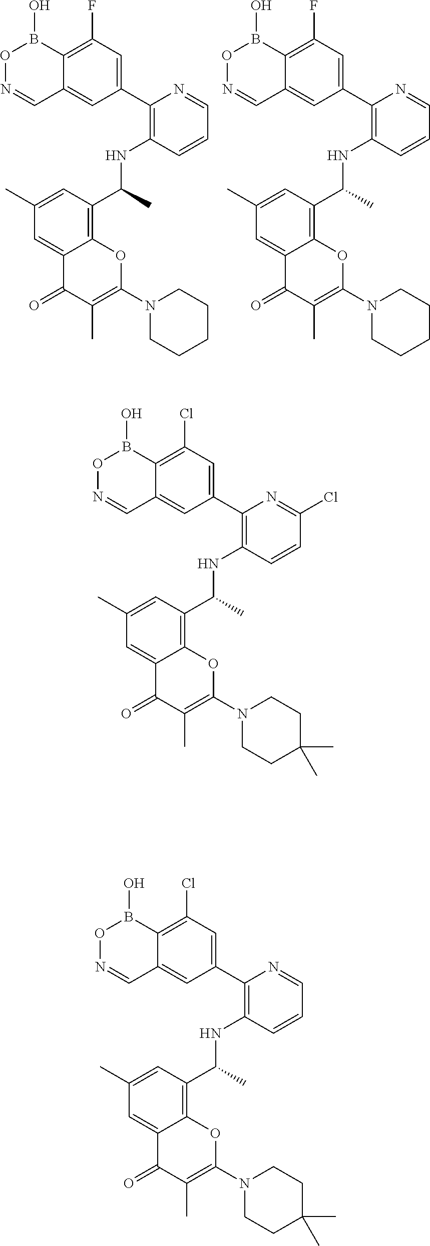

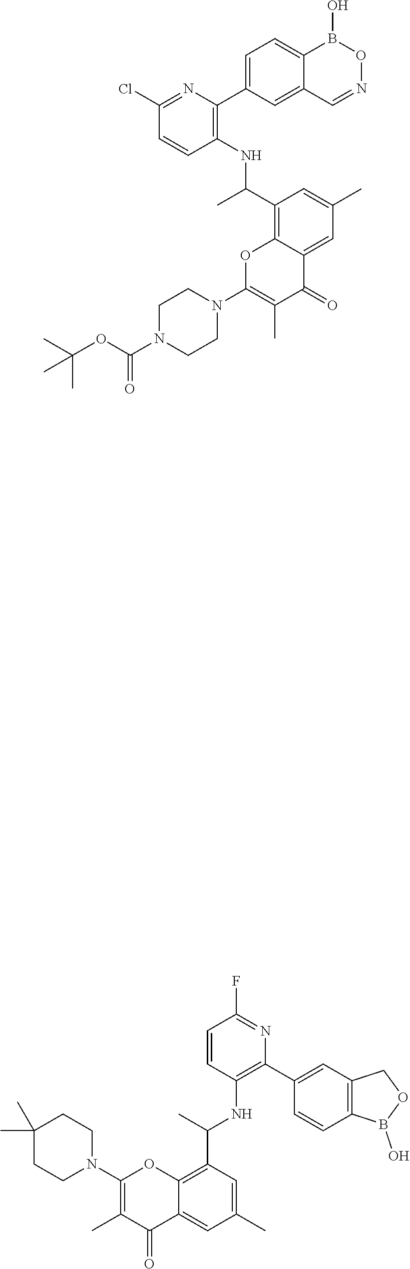 [SMILES: Cc1cc(C(C)Nc2ccc(Cl)nc2-c2ccc3c(c2)C=NOB3O)c2oc(N3CCN(C(=O)OC(C)(C)C)CC3)c(C)c(=O)c2c1 ; Cc1cc(C(C)Nc2ccc(F)nc2-c2ccc3c(c2)COB3O)c2oc(N3CCC(C)(C)CC3)c(C)c(=O)c2c1]

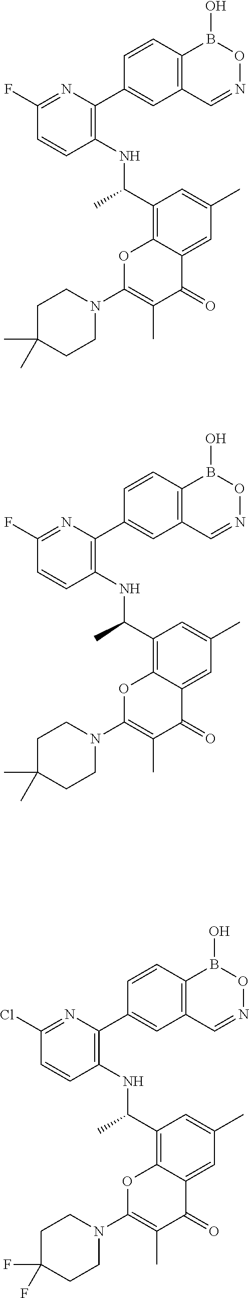 [SMILES: Cc1cc([C@H](C)Nc2ccc(F)nc2-c2ccc3c(c2)C=NOB3O)c2oc(N3CCC(C)(C)CC3)c(C)c(=O)c2c1 ; Cc1cc([C@@H](C)Nc2ccc(F)nc2-c2ccc3c(c2)C=NOB3O)c2oc(N3CCC(C)(C)CC3)c(C)c(=O)c2c1 ; Cc1cc([C@H](C)Nc2ccc(Cl)nc2-c2ccc3c(c2)C=NOB3O)c2oc(N3CCC(F)(F)CC3)c(C)c(=O)c2c1]

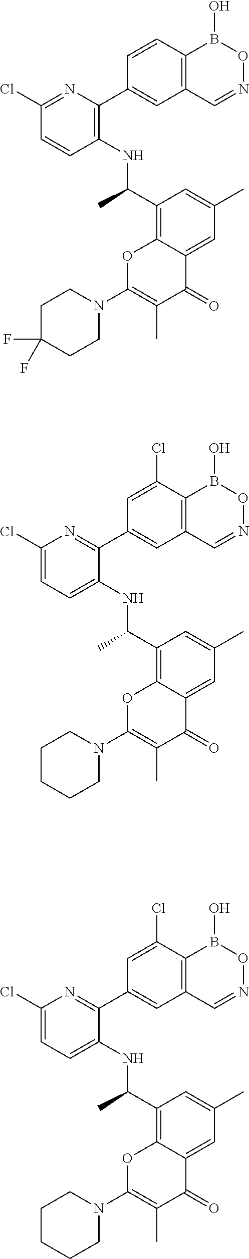 [SMILES: Cc1cc([C@@H](C)Nc2ccc(Cl)nc2-c2ccc3c(c2)C=NOB3O)c2oc(N3CCC(F)(F)CC3)c(C)c(=O)c2c1 ; Cc1cc([C@H](C)Nc2ccc(Cl)nc2-c2cc(Cl)c3c(c2)C=NOB3O)c2oc(N3CCCCC3)c(C)c(=O)c2c1 ; Cc1cc([C@@H](C)Nc2ccc(Cl)nc2-c2cc(Cl)c3c(c2)C=NOB3O)c2oc(N3CCCCC3)c(C)c(=O)c2c1]

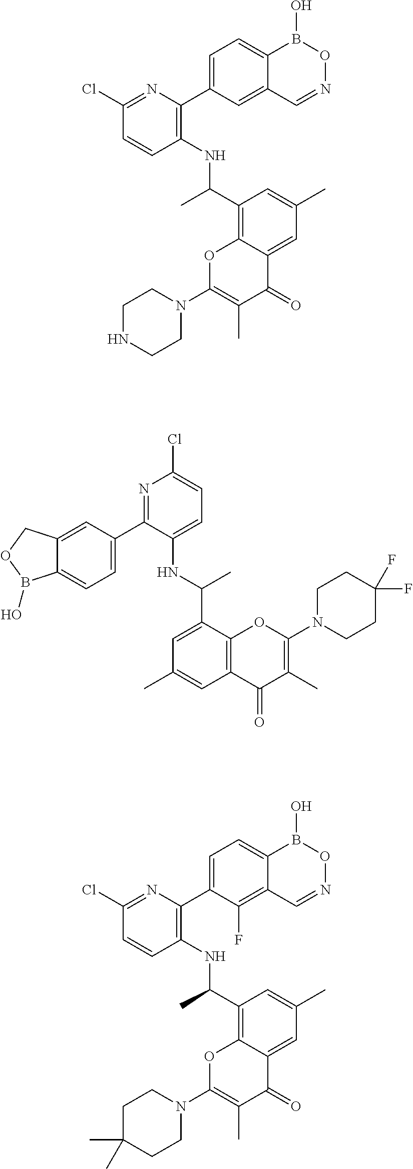

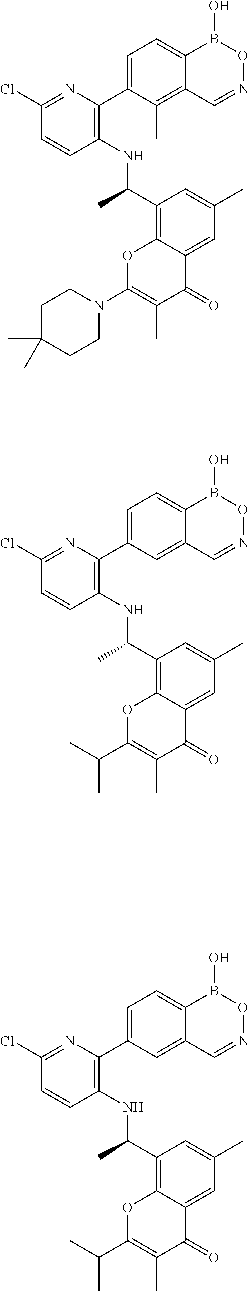

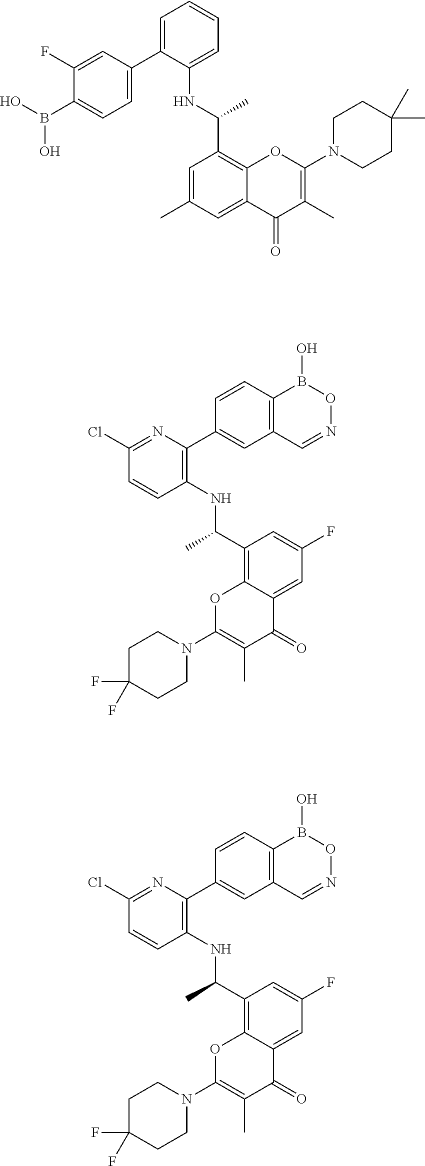 [SMILES: Cc1cc([C@@H](C)Nc2ccccc2-c2ccc(B(O)O)c(F)c2)c2oc(N3CCC(C)(C)CC3)c(C)c(=O)c2c1 ; Cc1c(N2CCC(F)(F)CC2)oc2c([C@H](C)Nc3ccc(Cl)nc3-c3ccc4c(c3)C=NOB4O)cc(F)cc2c1=O ; Cc1c(N2CCC(F)(F)CC2)oc2c([C@@H](C)Nc3ccc(Cl)nc3-c3ccc4c(c3)C=NOB4O)cc(F)cc2c1=O]

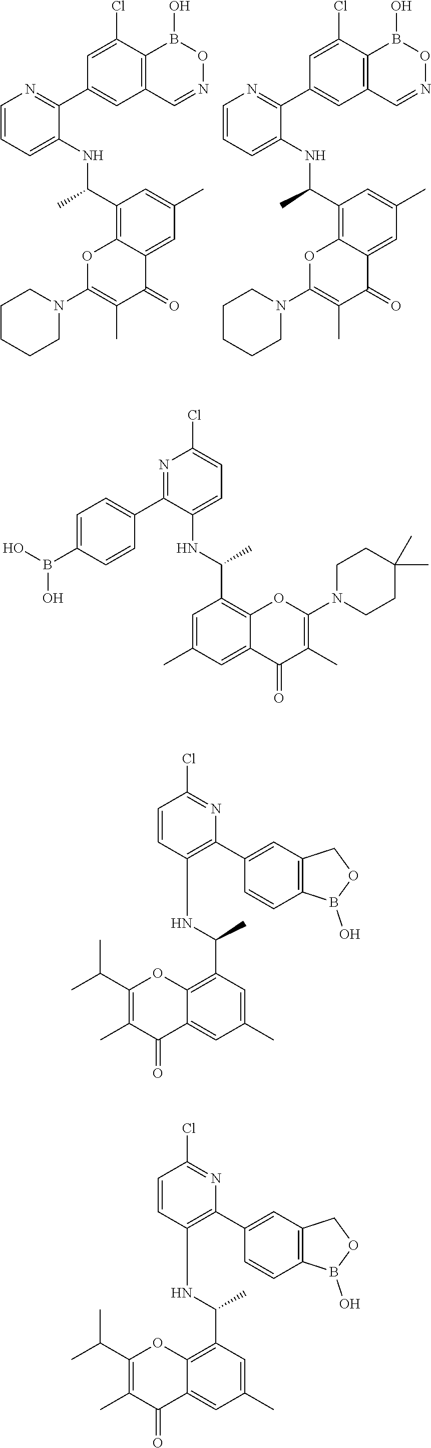

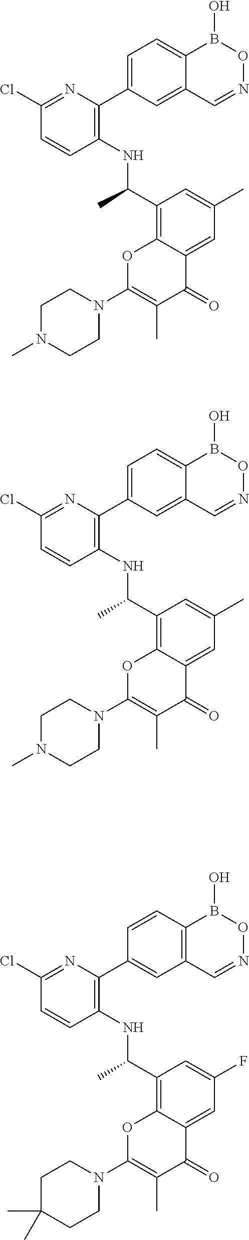

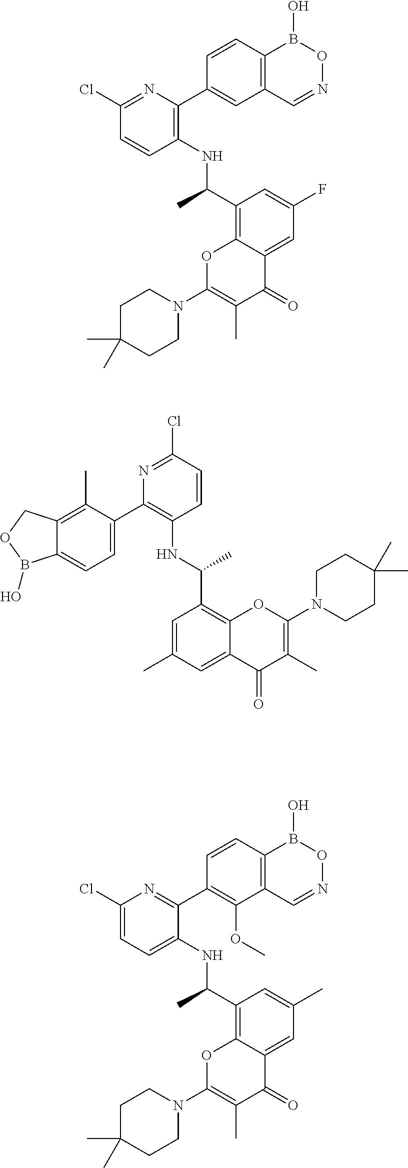

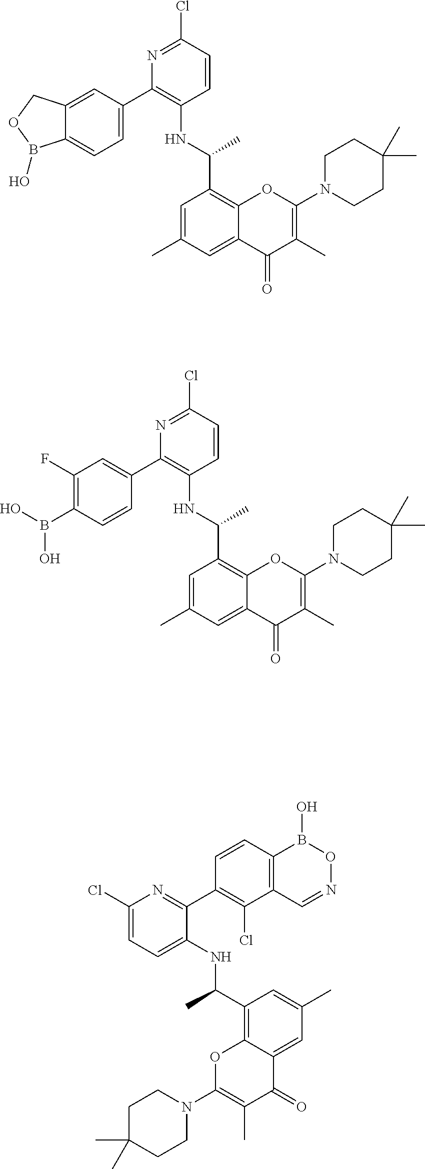

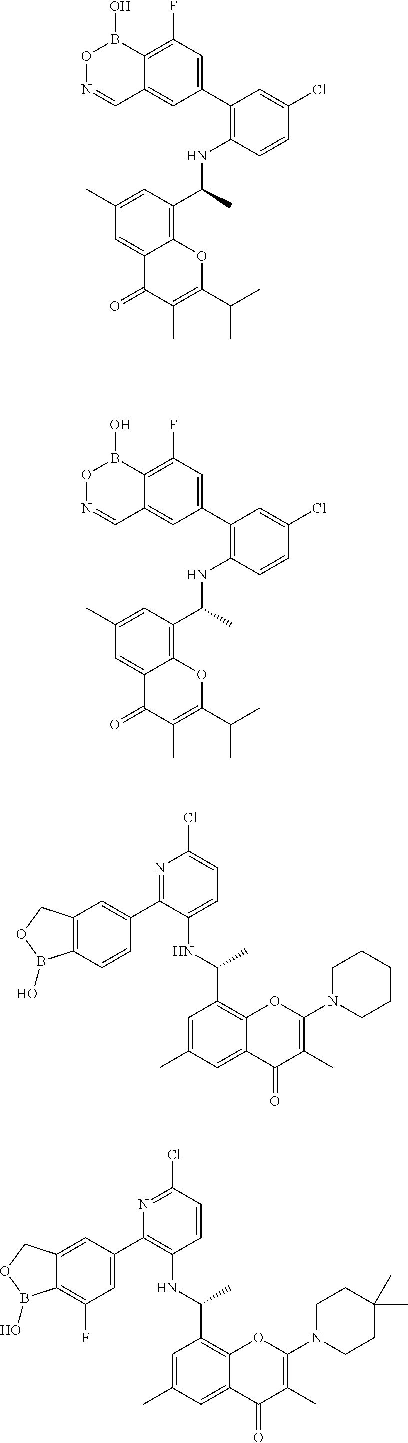 [SMILES: Cc1cc([C@H](C)Nc2ccc(Cl)cc2-c2cc(F)c3c(c2)C=NOB3O)c2oc(C(C)C)c(C)c(=O)c2c1 ; Cc1cc([C@@H](C)Nc2ccc(Cl)cc2-c2cc(F)c3c(c2)C=NOB3O)c2oc(C(C)C)c(C)c(=O)c2c1 ; Cc1cc([C@@H](C)Nc2ccc(Cl)nc2-c2ccc3c(c2)COB3O)c2oc(N3CCCCC3)c(C)c(=O)c2c1 ; Cc1cc([C@@H](C)Nc2ccc(Cl)nc2-c2cc(F)c3c(c2)COB3O)c2oc(N3CCC(C)(C)CC3)c(C)c(=O)c2c1]

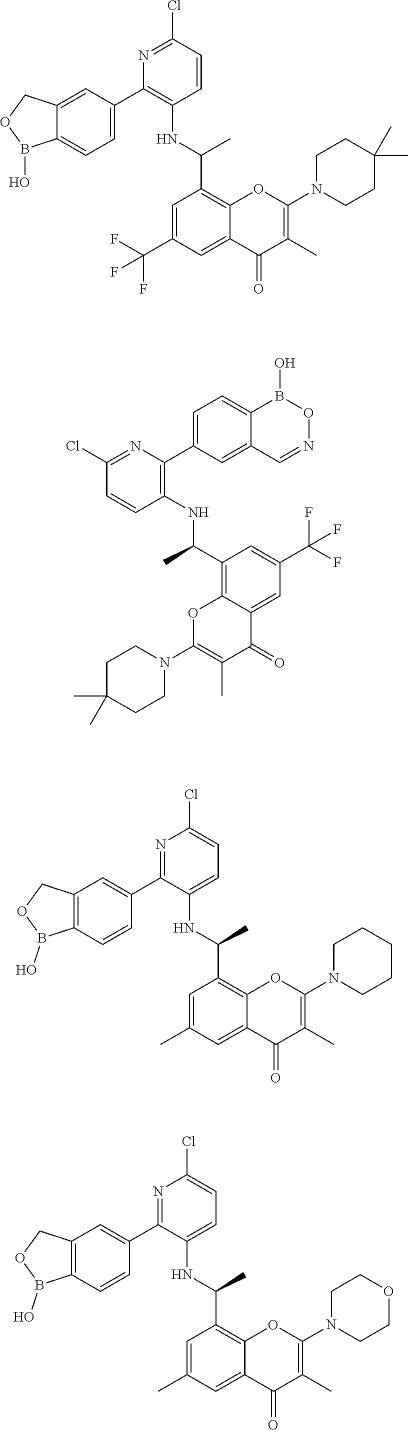

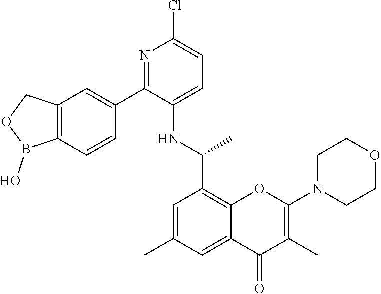 [SMILES: Cc1cc([C@@H](C)Nc2ccc(Cl)nc2-c2ccc3c(c2)COB3O)c2oc(N3CCOCC3)c(C)c(=O)c2c1]

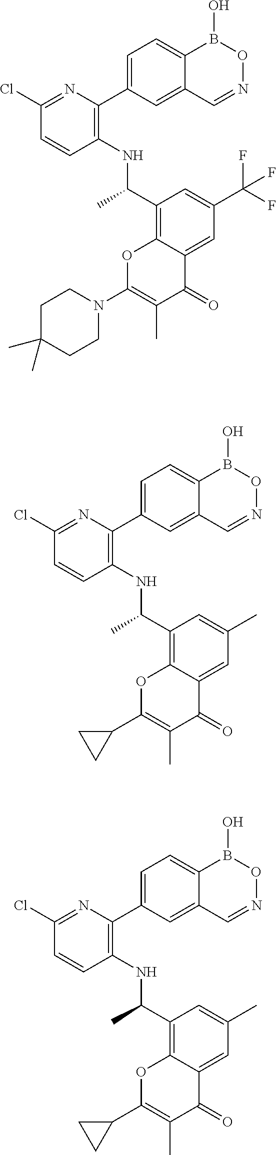 [SMILES: Cc1c(N2CCC(C)(C)CC2)oc2c([C@H](C)Nc3ccc(Cl)nc3-c3ccc4c(c3)C=NOB4O)cc(C(F)(F)F)cc2c1=O ; Cc1cc([C@H](C)Nc2ccc(Cl)nc2-c2ccc3c(c2)C=NOB3O)c2oc(C3CC3)c(C)c(=O)c2c1 ; Cc1cc([C@@H](C)Nc2ccc(Cl)nc2-c2ccc3c(c2)C=NOB3O)c2oc(C3CC3)c(C)c(=O)c2c1]

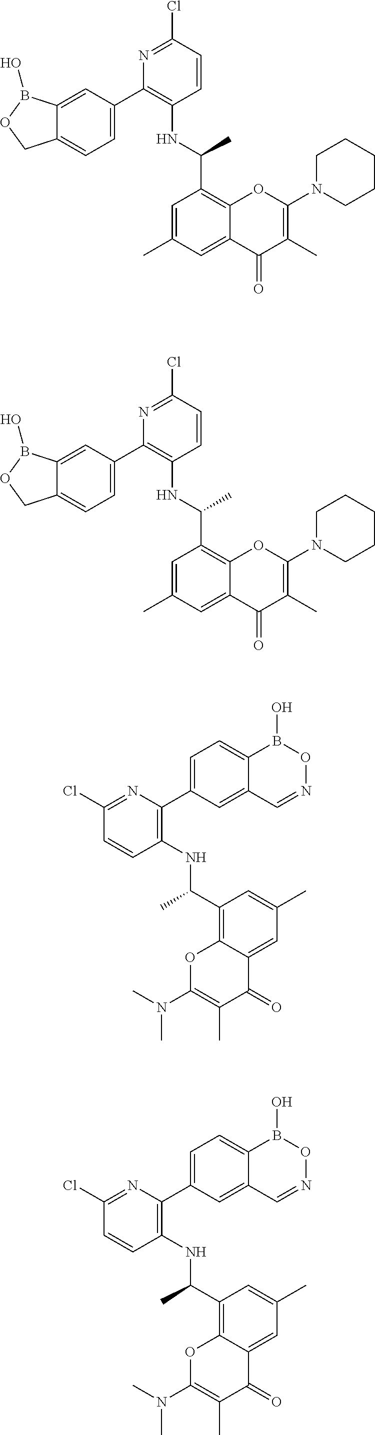 [SMILES: Cc1cc([C@H](C)Nc2ccc(Cl)nc2-c2ccc3c(c2)B(O)OC3)c2oc(N3CCCCC3)c(C)c(=O)c2c1 ; Cc1cc([C@@H](C)Nc2ccc(Cl)nc2-c2ccc3c(c2)B(O)OC3)c2oc(N3CCCCC3)c(C)c(=O)c2c1 ; Cc1cc([C@H](C)Nc2ccc(Cl)nc2-c2ccc3c(c2)C=NOB3O)c2oc(N(C)C)c(C)c(=O)c2c1 ; Cc1cc([C@@H](C)Nc2ccc(Cl)nc2-c2ccc3c(c2)C=NOB3O)c2oc(N(C)C)c(C)c(=O)c2c1]

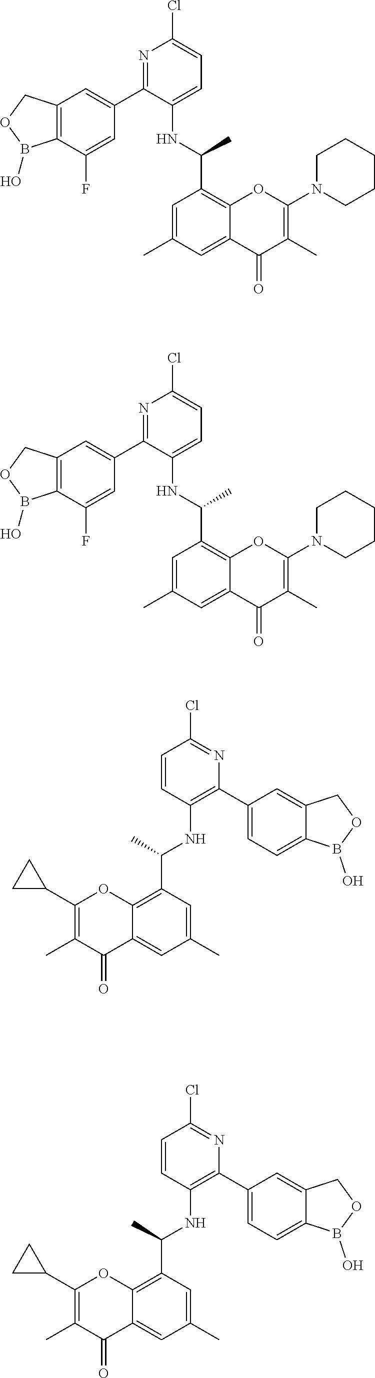 [SMILES: Cc1cc([C@H](C)Nc2ccc(Cl)nc2-c2cc(F)c3c(c2)COB3O)c2oc(N3CCCCC3)c(C)c(=O)c2c1 ; Cc1cc([C@@H](C)Nc2ccc(Cl)nc2-c2cc(F)c3c(c2)COB3O)c2oc(N3CCCCC3)c(C)c(=O)c2c1 ; Cc1cc([C@H](C)Nc2ccc(Cl)nc2-c2ccc3c(c2)COB3O)c2oc(C3CC3)c(C)c(=O)c2c1 ; Cc1cc([C@@H](C)Nc2ccc(Cl)nc2-c2ccc3c(c2)COB3O)c2oc(C3CC3)c(C)c(=O)c2c1]

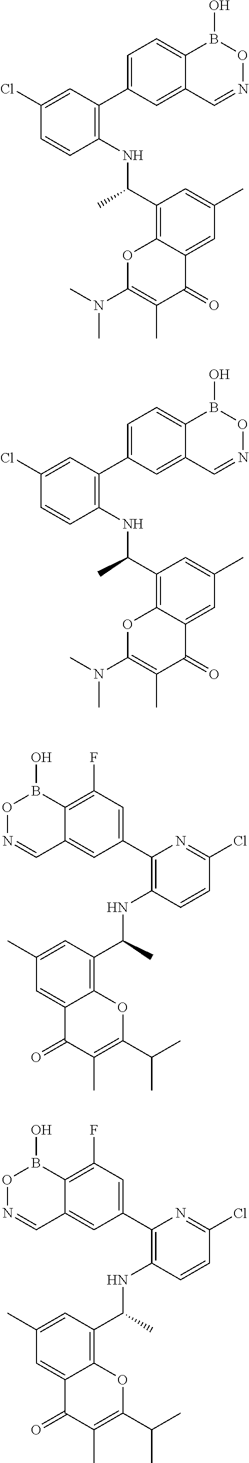

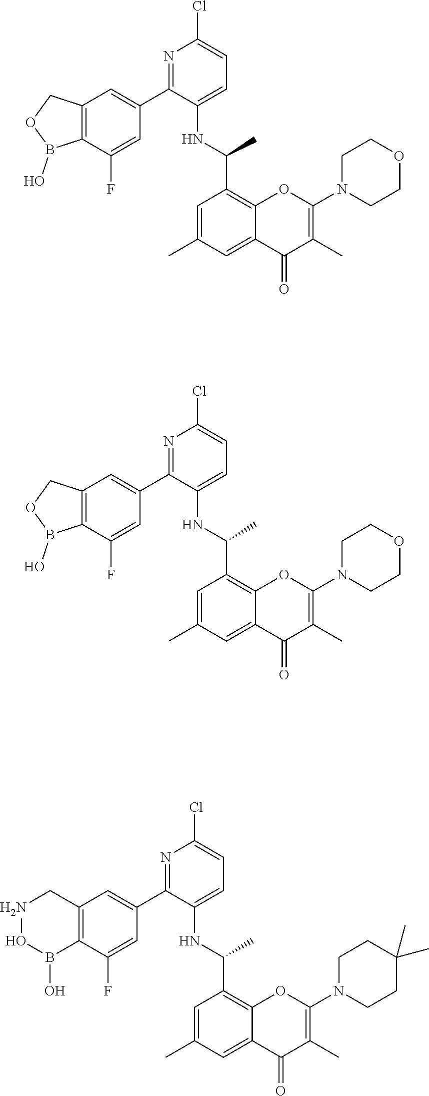 [SMILES: Cc1cc([C@H](C)Nc2ccc(Cl)nc2-c2cc(F)c3c(c2)COB3O)c2oc(N3CCOCC3)c(C)c(=O)c2c1 ; Cc1cc([C@@H](C)Nc2ccc(Cl)nc2-c2cc(F)c3c(c2)COB3O)c2oc(N3CCOCC3)c(C)c(=O)c2c1 ; Cc1cc([C@@H](C)Nc2ccc(Cl)nc2-c2cc(F)c3c(c2)CNOB3O)c2oc(N3CCC(C)(C)CC3)c(C)c(=O)c2c1]

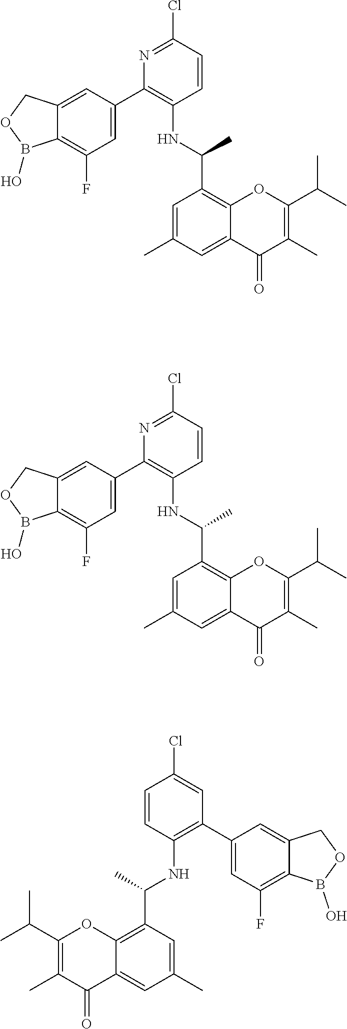

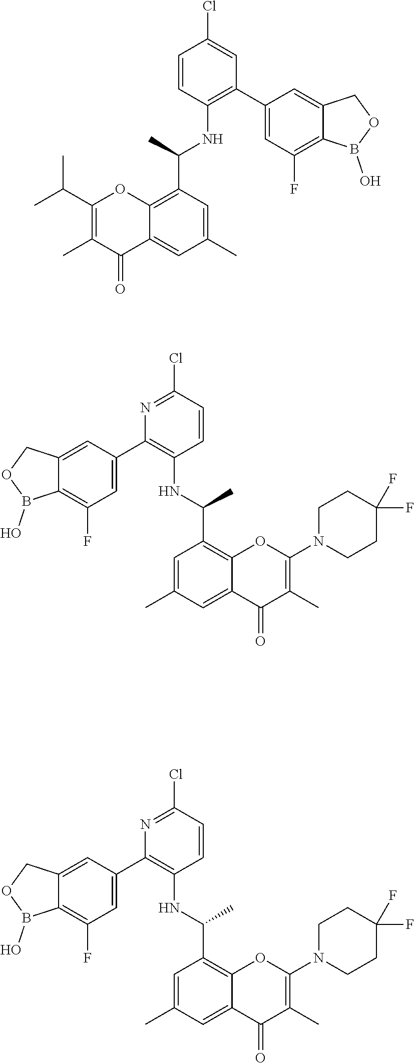 [SMILES: Cc1cc([C@@H](C)Nc2ccc(Cl)cc2-c2cc(F)c3c(c2)COB3O)c2oc(C(C)C)c(C)c(=O)c2c1 ; Cc1cc([C@H](C)Nc2ccc(Cl)nc2-c2cc(F)c3c(c2)COB3O)c2oc(N3CCC(F)(F)CC3)c(C)c(=O)c2c1 ; Cc1cc([C@@H](C)Nc2ccc(Cl)nc2-c2cc(F)c3c(c2)COB3O)c2oc(N3CCC(F)(F)CC3)c(C)c(=O)c2c1]

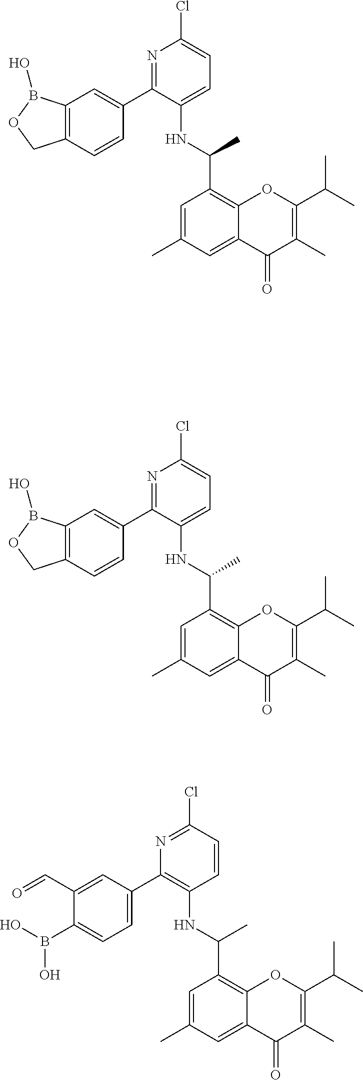 [SMILES: Cc1cc([C@H](C)Nc2ccc(Cl)nc2-c2ccc3c(c2)B(O)OC3)c2oc(C(C)C)c(C)c(=O)c2c1 ; Cc1cc([C@@H](C)Nc2ccc(Cl)nc2-c2ccc3c(c2)B(O)OC3)c2oc(C(C)C)c(C)c(=O)c2c1 ; Cc1cc(C(C)Nc2ccc(Cl)nc2-c2ccc(B(O)O)c(C=O)c2)c2oc(C(C)C)c(C)c(=O)c2c1]

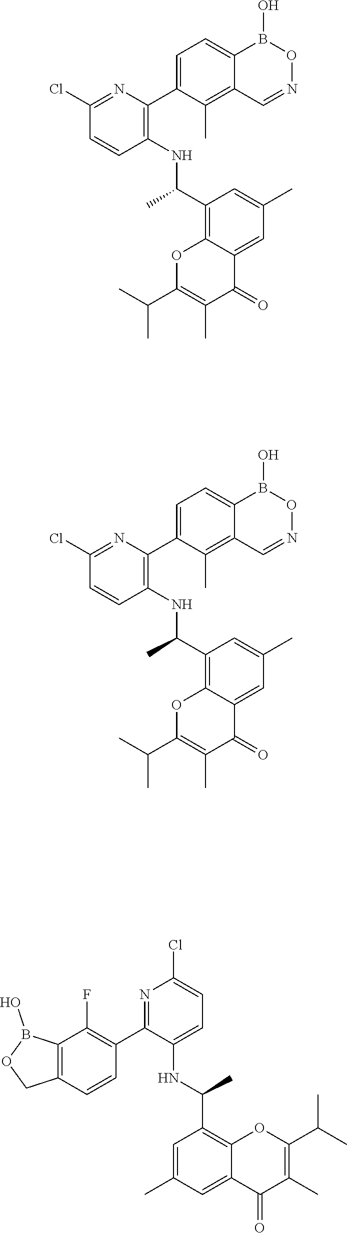 [SMILES: Cc1cc([C@H](C)Nc2ccc(Cl)nc2-c2ccc3c(c2C)C=NOB3O)c2oc(C(C)C)c(C)c(=O)c2c1 ; Cc1cc([C@@H](C)Nc2ccc(Cl)nc2-c2ccc3c(c2C)C=NOB3O)c2oc(C(C)C)c(C)c(=O)c2c1 ; Cc1cc([C@H](C)Nc2ccc(Cl)nc2-c2ccc3c(c2F)B(O)OC3)c2oc(C(C)C)c(C)c(=O)c2c1]

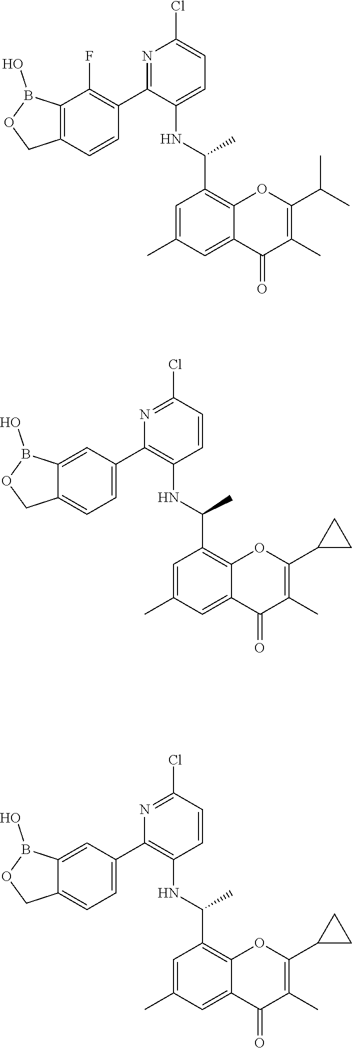 [SMILES: Cc1cc([C@@H](C)Nc2ccc(Cl)nc2-c2ccc3c(c2F)B(O)OC3)c2oc(C(C)C)c(C)c(=O)c2c1 ; Cc1cc([C@H](C)Nc2ccc(Cl)nc2-c2ccc3c(c2)B(O)OC3)c2oc(C3CC3)c(C)c(=O)c2c1 ; Cc1cc([C@@H](C)Nc2ccc(Cl)nc2-c2ccc3c(c2)B(O)OC3)c2oc(C3CC3)c(C)c(=O)c2c1]

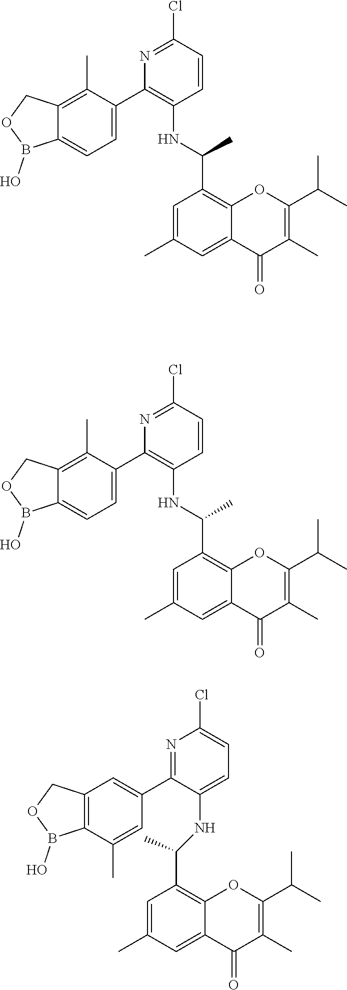 [SMILES: Cc1cc([C@H](C)Nc2ccc(Cl)nc2-c2ccc3c(c2C)COB3O)c2oc(C(C)C)c(C)c(=O)c2c1 ; Cc1cc([C@@H](C)Nc2ccc(Cl)nc2-c2ccc3c(c2C)COB3O)c2oc(C(C)C)c(C)c(=O)c2c1 ; Cc1cc([C@H](C)Nc2ccc(Cl)nc2-c2cc(C)c3c(c2)COB3O)c2oc(C(C)C)c(C)c(=O)c2c1]

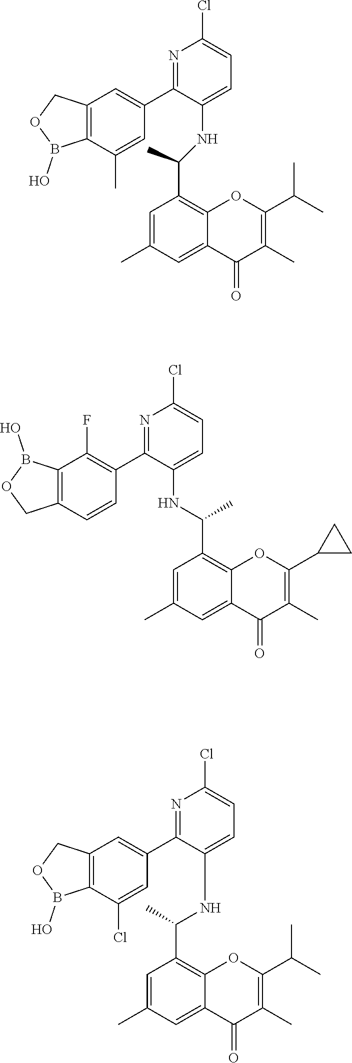 [SMILES: Cc1cc([C@@H](C)Nc2ccc(Cl)nc2-c2cc(C)c3c(c2)COB3O)c2oc(C(C)C)c(C)c(=O)c2c1 ; Cc1cc([C@@H](C)Nc2ccc(Cl)nc2-c2ccc3c(c2F)B(O)OC3)c2oc(C3CC3)c(C)c(=O)c2c1 ; Cc1cc([C@H](C)Nc2ccc(Cl)nc2-c2cc(Cl)c3c(c2)COB3O)c2oc(C(C)C)c(C)c(=O)c2c1]

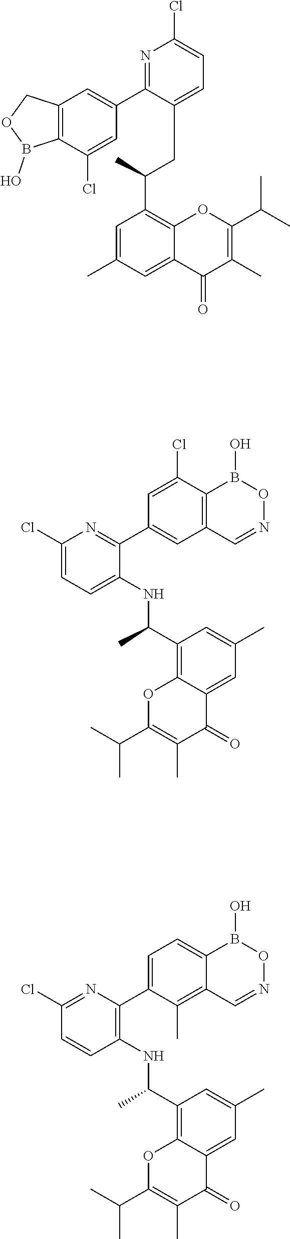 [SMILES: Cc1cc([C@@H](C)Cc2ccc(Cl)nc2-c2cc(Cl)c3c(c2)COB3O)c2oc(C(C)C)c(C)c(=O)c2c1 ; Cc1cc([C@@H](C)Nc2ccc(Cl)nc2-c2cc(Cl)c3c(c2)C=NOB3O)c2oc(C(C)C)c(C)c(=O)c2c1 ; Cc1cc([C@H](C)Nc2ccc(Cl)nc2-c2ccc3c(c2C)C=NOB3O)c2oc(C(C)C)c(C)c(=O)c2c1]

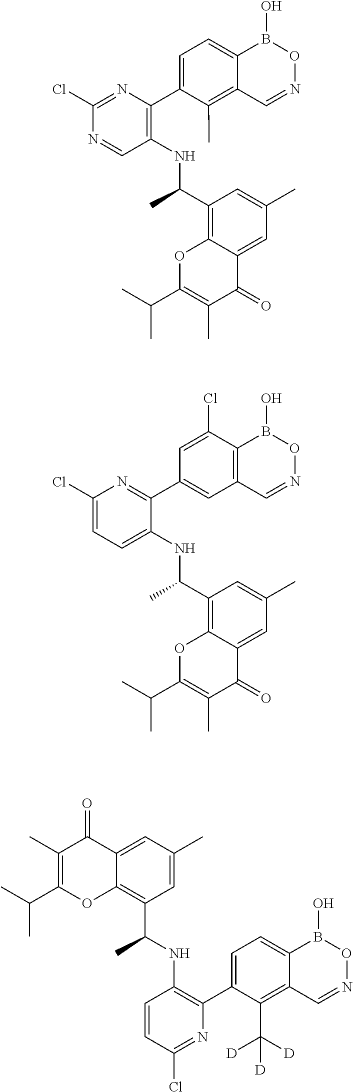

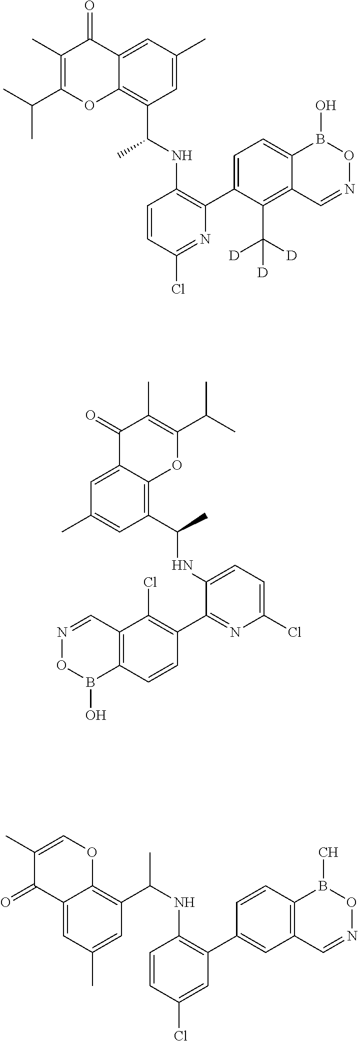 [SMILES: [2H]C([2H])([2H])c1c(-c2nc(Cl)ccc2N[C@H](C)c2cc(C)cc3c(=O)c(C)c(C(C)C)oc23)ccc2c1C=NOB2O ; Cc1cc([C@@H](C)Nc2ccc(Cl)nc2-c2ccc3c(c2Cl)C=NOB3O)c2oc(C(C)C)c(C)c(=O)c2c1 ; CB1ON=Cc2cc(-c3cc(Cl)ccc3NC(C)c3cc(C)cc4c(=O)c(C)coc34)ccc21]

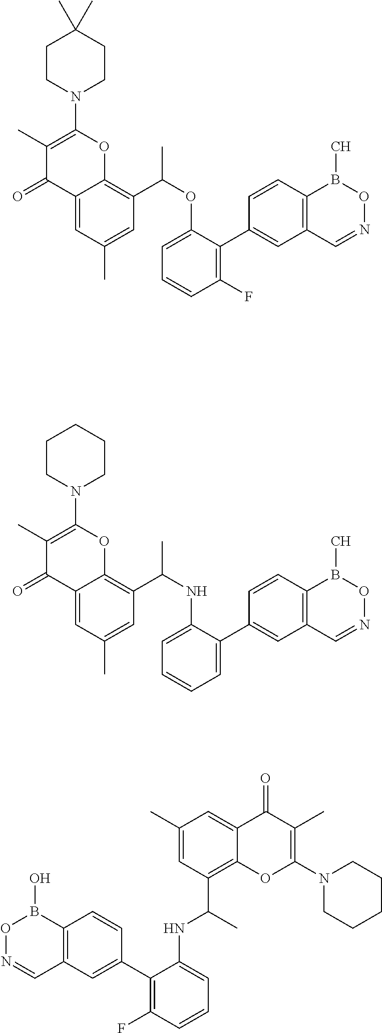 [SMILES: CB1ON=Cc2cc(-c3c(F)cccc3OC(C)c3cc(C)cc4c(=O)c(C)c(N5CCC(C)(C)CC5)oc34)ccc21 ; CB1ON=Cc2cc(-c3ccccc3NC(C)c3cc(C)cc4c(=O)c(C)c(N5CCCCC5)oc34)ccc21 ; Cc1cc(C(C)Nc2cccc(F)c2-c2ccc3c(c2)C=NOB3O)c2oc(N3CCCCC3)c(C)c(=O)c2c1]

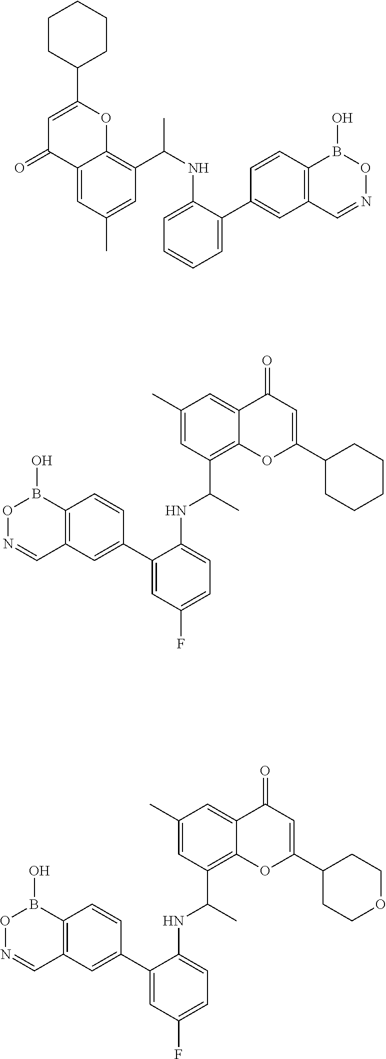 [SMILES: Cc1cc(C(C)Nc2ccccc2-c2ccc3c(c2)C=NOB3O)c2oc(C3CCCCC3)cc(=O)c2c1 ; Cc1cc(C(C)Nc2ccc(F)cc2-c2ccc3c(c2)C=NOB3O)c2oc(C3CCCCC3)cc(=O)c2c1 ; Cc1cc(C(C)Nc2ccc(F)cc2-c2ccc3c(c2)C=NOB3O)c2oc(C3CCOCC3)cc(=O)c2c1]

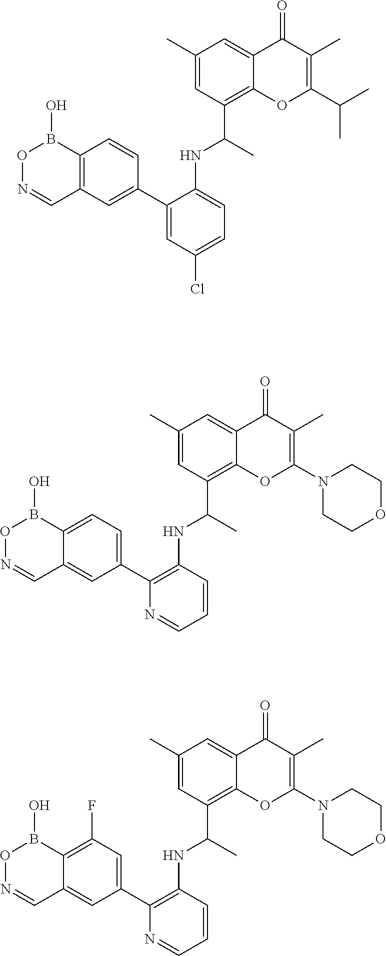 [SMILES: Cc1cc(C(C)Nc2ccc(Cl)cc2-c2ccc3c(c2)C=NOB3O)c2oc(C(C)C)c(C)c(=O)c2c1 ; Cc1cc(C(C)Nc2cccnc2-c2ccc3c(c2)C=NOB3O)c2oc(N3CCOCC3)c(C)c(=O)c2c1 ; Cc1cc(C(C)Nc2cccnc2-c2cc(F)c3c(c2)C=NOB3O)c2oc(N3CCOCC3)c(C)c(=O)c2c1]

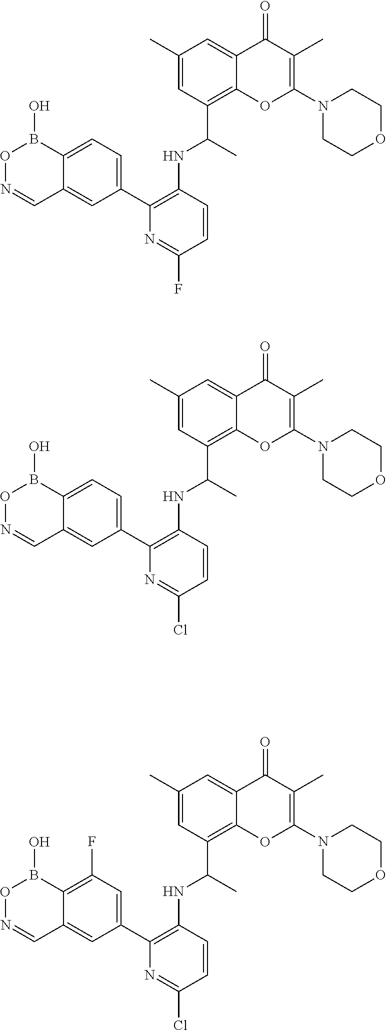 [SMILES: Cc1cc(C(C)Nc2ccc(F)nc2-c2ccc3c(c2)C=NOB3O)c2oc(N3CCOCC3)c(C)c(=O)c2c1 ; Cc1cc(C(C)Nc2ccc(Cl)nc2-c2ccc3c(c2)C=NOB3O)c2oc(N3CCOCC3)c(C)c(=O)c2c1 ; Cc1cc(C(C)Nc2ccc(Cl)nc2-c2cc(F)c3c(c2)C=NOB3O)c2oc(N3CCOCC3)c(C)c(=O)c2c1]

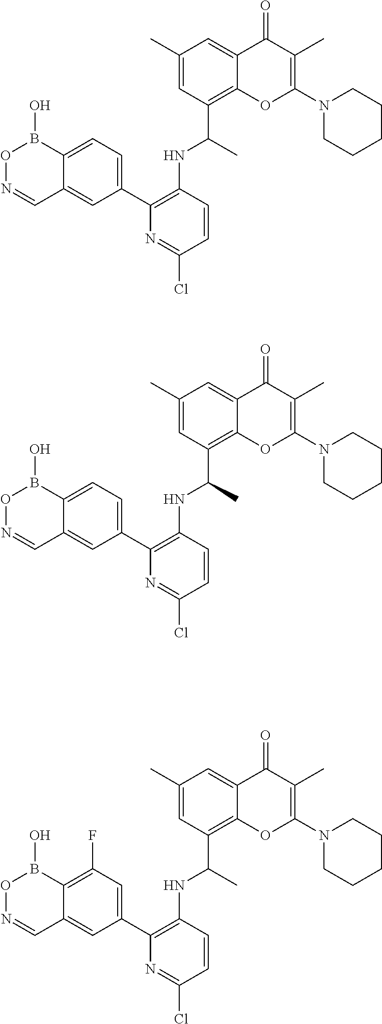 [SMILES: Cc1cc(C(C)Nc2ccc(Cl)nc2-c2ccc3c(c2)C=NOB3O)c2oc(N3CCCCC3)c(C)c(=O)c2c1 ; Cc1cc([C@@H](C)Nc2ccc(Cl)nc2-c2ccc3c(c2)C=NOB3O)c2oc(N3CCCCC3)c(C)c(=O)c2c1 ; Cc1cc(C(C)Nc2ccc(Cl)nc2-c2cc(F)c3c(c2)C=NOB3O)c2oc(N3CCCCC3)c(C)c(=O)c2c1]

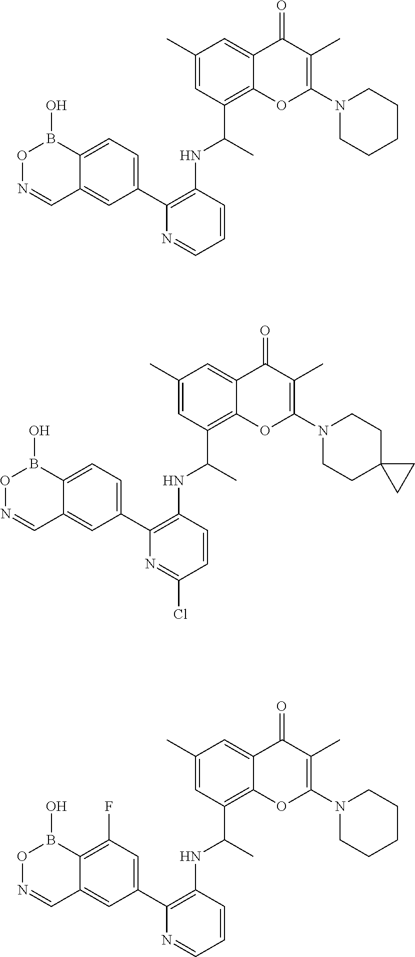 [SMILES: Cc1cc(C(C)Nc2cccnc2-c2ccc3c(c2)C=NOB3O)c2oc(N3CCCCC3)c(C)c(=O)c2c1 ; Cc1cc(C(C)Nc2ccc(Cl)nc2-c2ccc3c(c2)C=NOB3O)c2oc(N3CCC4(CC3)CC4)c(C)c(=O)c2c1 ; Cc1cc(C(C)Nc2cccnc2-c2cc(F)c3c(c2)C=NOB3O)c2oc(N3CCCCC3)c(C)c(=O)c2c1]

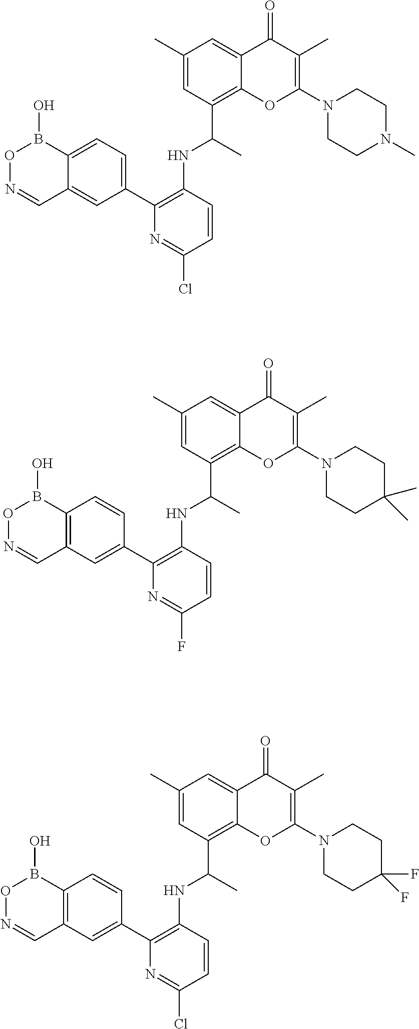 [SMILES: Cc1cc(C(C)Nc2ccc(Cl)nc2-c2ccc3c(c2)C=NOB3O)c2oc(N3CCN(C)CC3)c(C)c(=O)c2c1 ; Cc1cc(C(C)Nc2ccc(F)nc2-c2ccc3c(c2)C=NOB3O)c2oc(N3CCC(C)(C)CC3)c(C)c(=O)c2c1 ; Cc1cc(C(C)Nc2ccc(Cl)nc2-c2ccc3c(c2)C=NOB3O)c2oc(N3CCC(F)(F)CC3)c(C)c(=O)c2c1]

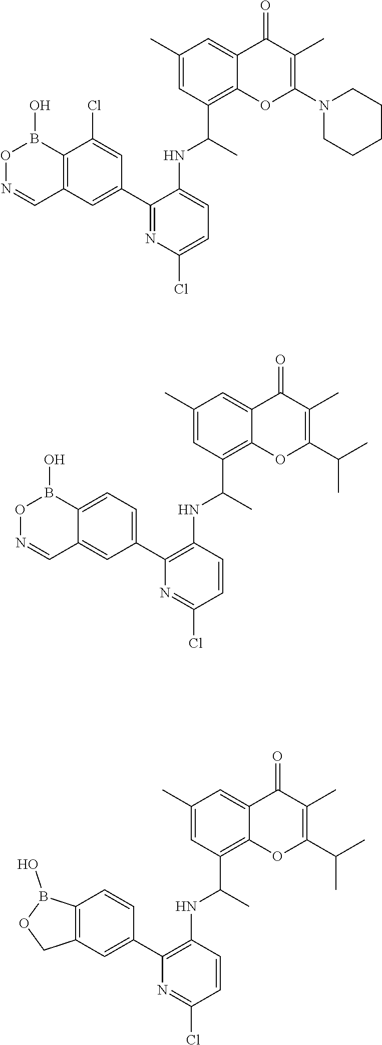 [SMILES: Cc1cc(C(C)Nc2ccc(Cl)nc2-c2cc(Cl)c3c(c2)C=NOB3O)c2oc(N3CCCCC3)c(C)c(=O)c2c1 ; Cc1cc(C(C)Nc2ccc(Cl)nc2-c2ccc3c(c2)C=NOB3O)c2oc(C(C)C)c(C)c(=O)c2c1 ; Cc1cc(C(C)Nc2ccc(Cl)nc2-c2ccc3c(c2)COB3O)c2oc(C(C)C)c(C)c(=O)c2c1]

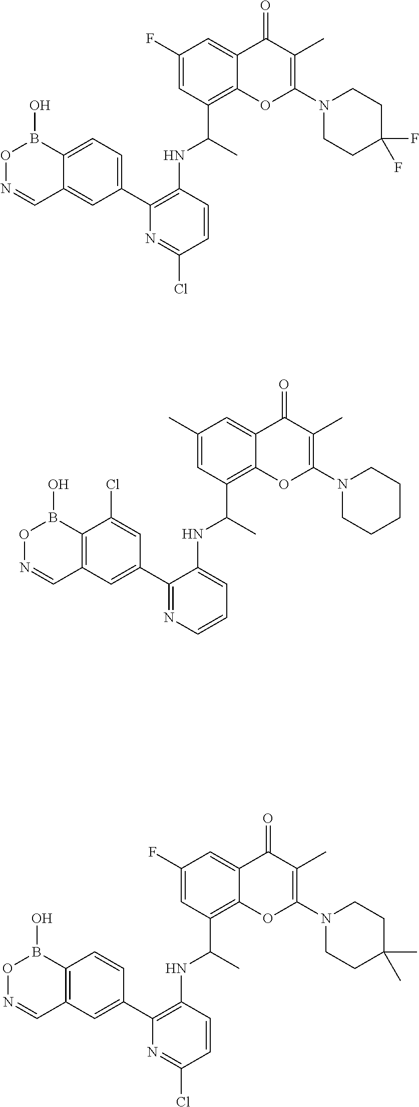 [SMILES: Cc1c(N2CCC(F)(F)CC2)oc2c(C(C)Nc3ccc(Cl)nc3-c3ccc4c(c3)C=NOB4O)cc(F)cc2c1=O ; Cc1cc(C(C)Nc2cccnc2-c2cc(Cl)c3c(c2)C=NOB3O)c2oc(N3CCCCC3)c(C)c(=O)c2c1 ; Cc1c(N2CCC(C)(C)CC2)oc2c(C(C)Nc3ccc(Cl)nc3-c3ccc4c(c3)C=NOB4O)cc(F)cc2c1=O]

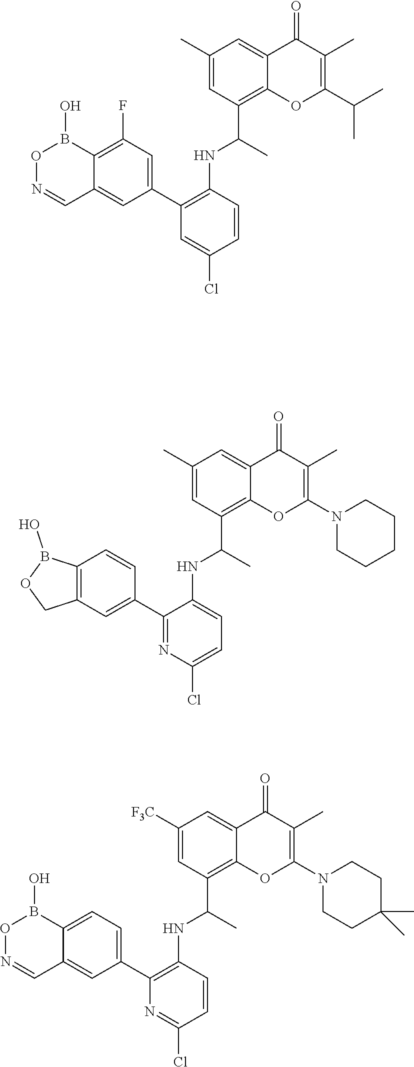 [SMILES: Cc1cc(C(C)Nc2ccc(Cl)cc2-c2cc(F)c3c(c2)C=NOB3O)c2oc(C(C)C)c(C)c(=O)c2c1 ; Cc1cc(C(C)Nc2ccc(Cl)nc2-c2ccc3c(c2)COB3O)c2oc(N3CCCCC3)c(C)c(=O)c2c1 ; Cc1c(N2CCC(C)(C)CC2)oc2c(C(C)Nc3ccc(Cl)nc3-c3ccc4c(c3)C=NOB4O)cc(C(F)(F)F)cc2c1=O]

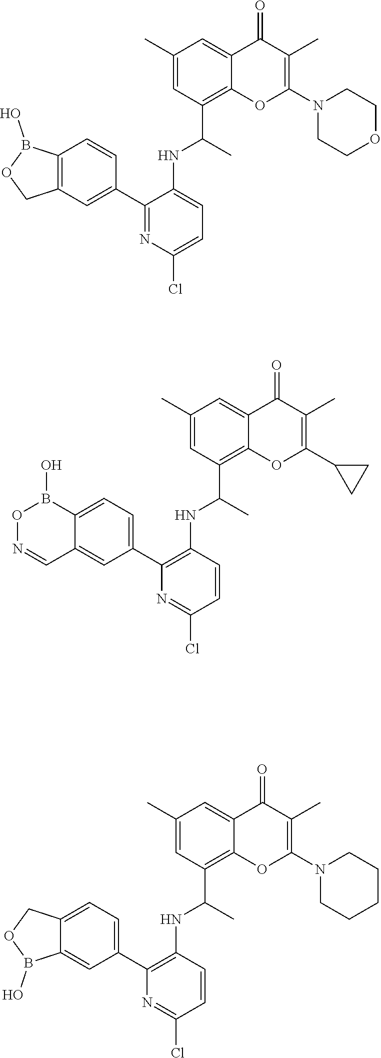 [SMILES: Cc1cc(C(C)Nc2ccc(Cl)nc2-c2ccc3c(c2)COB3O)c2oc(N3CCOCC3)c(C)c(=O)c2c1 ; Cc1cc(C(C)Nc2ccc(Cl)nc2-c2ccc3c(c2)C=NOB3O)c2oc(C3CC3)c(C)c(=O)c2c1 ; Cc1cc(C(C)Nc2ccc(Cl)nc2-c2ccc3c(c2)B(O)OC3)c2oc(N3CCCCC3)c(C)c(=O)c2c1]

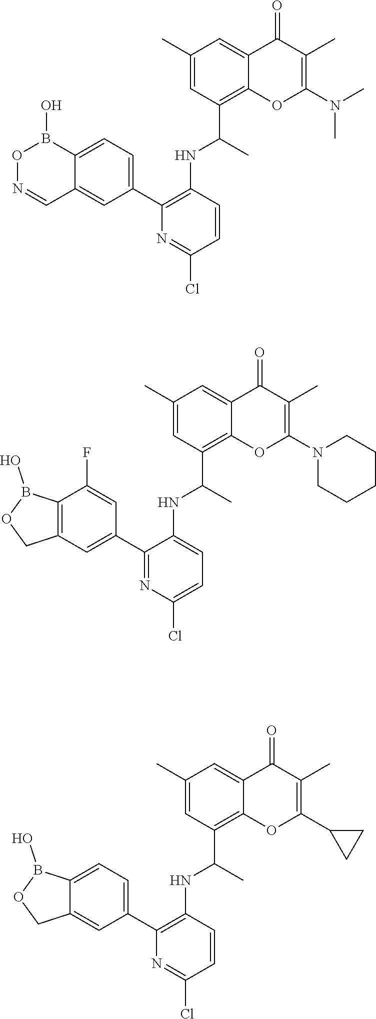 [SMILES: Cc1cc(C(C)Nc2ccc(Cl)nc2-c2ccc3c(c2)C=NOB3O)c2oc(N(C)C)c(C)c(=O)c2c1 ; Cc1cc(C(C)Nc2ccc(Cl)nc2-c2cc(F)c3c(c2)COB3O)c2oc(N3CCCCC3)c(C)c(=O)c2c1 ; Cc1cc(C(C)Nc2ccc(Cl)nc2-c2ccc3c(c2)COB3O)c2oc(C3CC3)c(C)c(=O)c2c1]

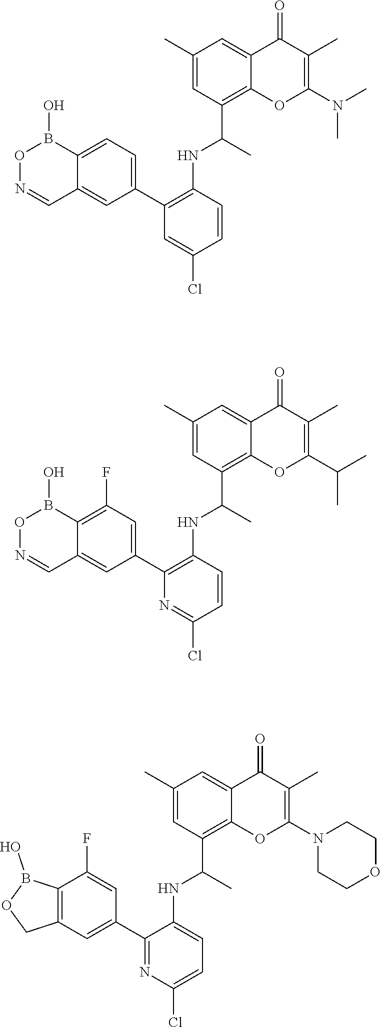 [SMILES: Cc1cc(C(C)Nc2ccc(Cl)cc2-c2ccc3c(c2)C=NOB3O)c2oc(N(C)C)c(C)c(=O)c2c1 ; Cc1cc(C(C)Nc2ccc(Cl)nc2-c2cc(F)c3c(c2)C=NOB3O)c2oc(C(C)C)c(C)c(=O)c2c1 ; Cc1cc(C(C)Nc2ccc(Cl)nc2-c2cc(F)c3c(c2)COB3O)c2oc(N3CCOCC3)c(C)c(=O)c2c1]

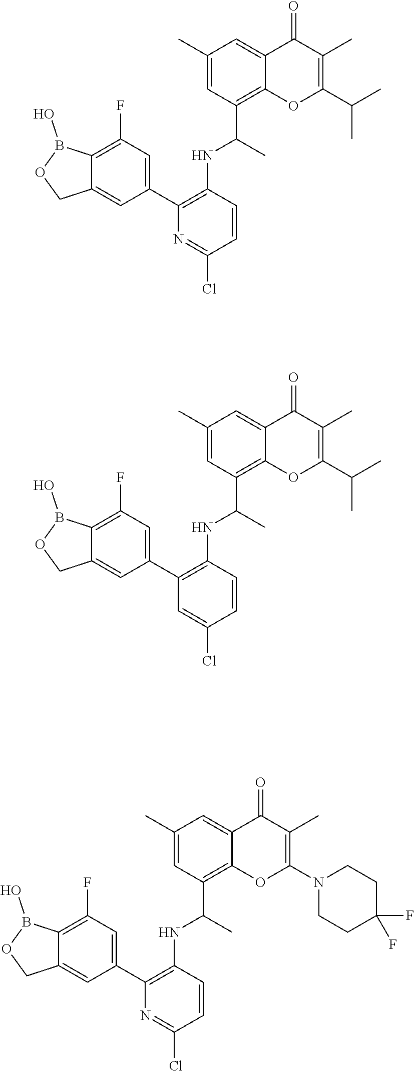 [SMILES: Cc1cc(C(C)Nc2ccc(Cl)nc2-c2cc(F)c3c(c2)COB3O)c2oc(C(C)C)c(C)c(=O)c2c1 ; Cc1cc(C(C)Nc2ccc(Cl)cc2-c2cc(F)c3c(c2)COB3O)c2oc(C(C)C)c(C)c(=O)c2c1 ; Cc1cc(C(C)Nc2ccc(Cl)nc2-c2cc(F)c3c(c2)COB3O)c2oc(N3CCC(F)(F)CC3)c(C)c(=O)c2c1]

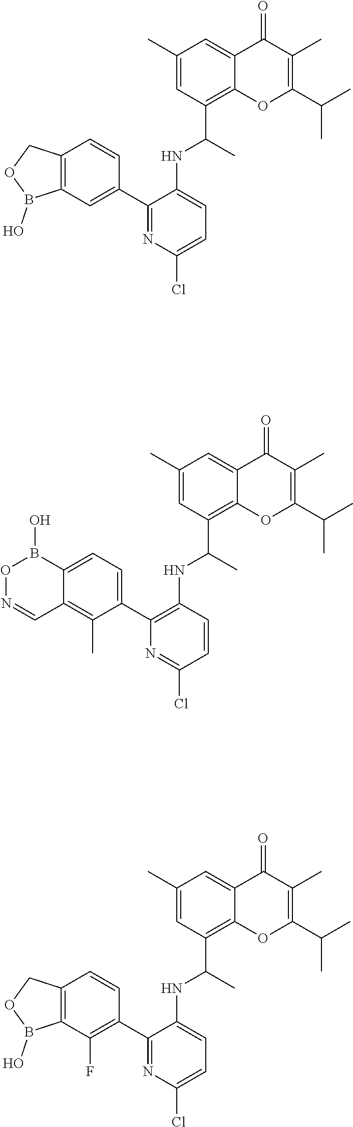 [SMILES: Cc1cc(C(C)Nc2ccc(Cl)nc2-c2ccc3c(c2)B(O)OC3)c2oc(C(C)C)c(C)c(=O)c2c1 ; Cc1cc(C(C)Nc2ccc(Cl)nc2-c2ccc3c(c2C)C=NOB3O)c2oc(C(C)C)c(C)c(=O)c2c1 ; Cc1cc(C(C)Nc2ccc(Cl)nc2-c2ccc3c(c2F)B(O)OC3)c2oc(C(C)C)c(C)c(=O)c2c1]

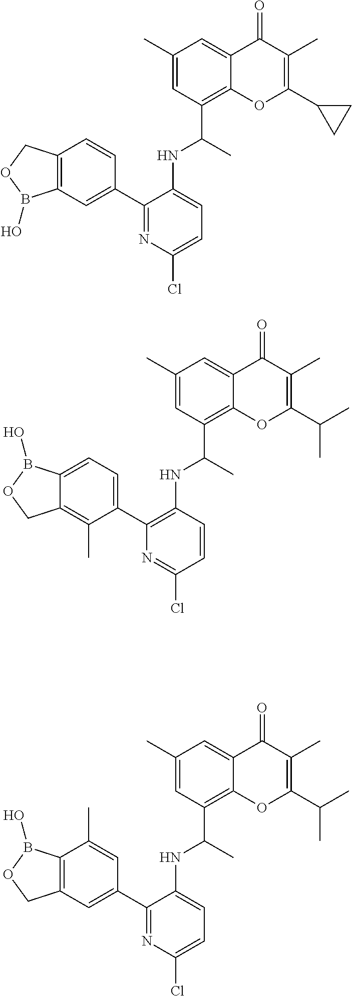 [SMILES: Cc1cc(C(C)Nc2ccc(Cl)nc2-c2ccc3c(c2)B(O)OC3)c2oc(C3CC3)c(C)c(=O)c2c1 ; Cc1cc(C(C)Nc2ccc(Cl)nc2-c2ccc3c(c2C)COB3O)c2oc(C(C)C)c(C)c(=O)c2c1 ; Cc1cc(C(C)Nc2ccc(Cl)nc2-c2cc(C)c3c(c2)COB3O)c2oc(C(C)C)c(C)c(=O)c2c1]

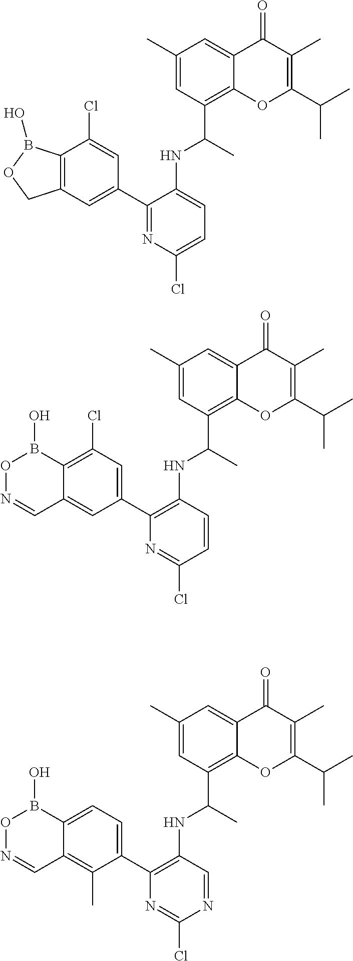 [SMILES: Cc1cc(C(C)Nc2ccc(Cl)nc2-c2cc(Cl)c3c(c2)COB3O)c2oc(C(C)C)c(C)c(=O)c2c1 ; Cc1cc(C(C)Nc2ccc(Cl)nc2-c2cc(Cl)c3c(c2)C=NOB3O)c2oc(C(C)C)c(C)c(=O)c2c1 ; Cc1cc(C(C)Nc2cnc(Cl)nc2-c2ccc3c(c2C)C=NOB3O)c2oc(C(C)C)c(C)c(=O)c2c1]

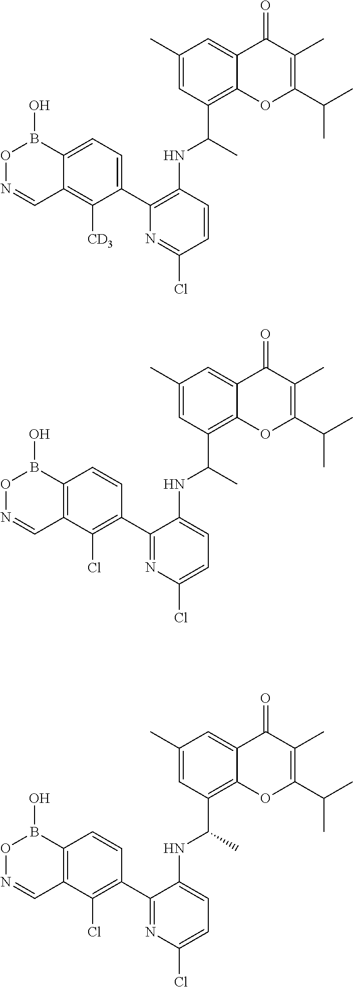 [SMILES: [2H]C([2H])([2H])c1c(-c2nc(Cl)ccc2NC(C)c2cc(C)cc3c(=O)c(C)c(C(C)C)oc23)ccc2c1C=NOB2O ; Cc1cc(C(C)Nc2ccc(Cl)nc2-c2ccc3c(c2Cl)C=NOB3O)c2oc(C(C)C)c(C)c(=O)c2c1 ; Cc1cc([C@H](C)Nc2ccc(Cl)nc2-c2ccc3c(c2Cl)C=NOB3O)c2oc(C(C)C)c(C)c(=O)c2c1]

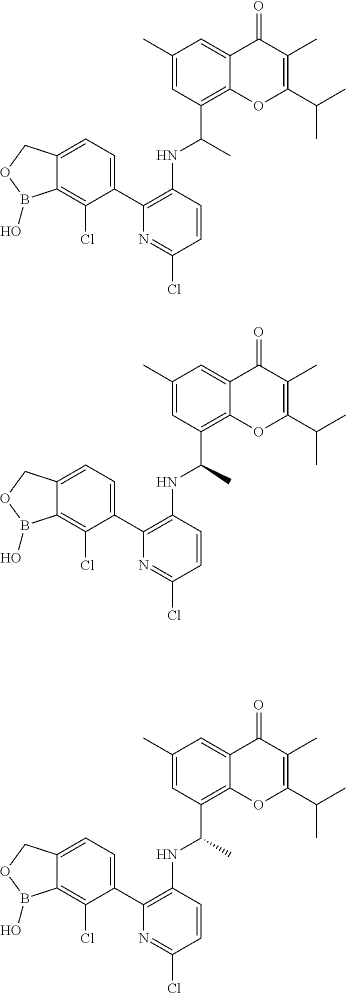 [SMILES: Cc1cc(C(C)Nc2ccc(Cl)nc2-c2ccc3c(c2Cl)B(O)OC3)c2oc(C(C)C)c(C)c(=O)c2c1 ; Cc1cc([C@@H](C)Nc2ccc(Cl)nc2-c2ccc3c(c2Cl)B(O)OC3)c2oc(C(C)C)c(C)c(=O)c2c1 ; Cc1cc([C@H](C)Nc2ccc(Cl)nc2-c2ccc3c(c2Cl)B(O)OC3)c2oc(C(C)C)c(C)c(=O)c2c1]

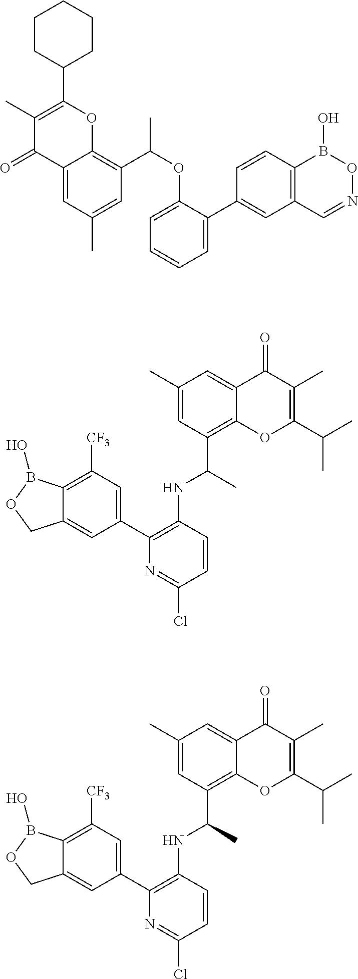 [SMILES: Cc1cc(C(C)Oc2ccccc2-c2ccc3c(c2)C=NOB3O)c2oc(C3CCCCC3)c(C)c(=O)c2c1 ; Cc1cc(C(C)Nc2ccc(Cl)nc2-c2cc3c(c(C(F)(F)F)c2)B(O)OC3)c2oc(C(C)C)c(C)c(=O)c2c1 ; Cc1cc([C@@H](C)Nc2ccc(Cl)nc2-c2cc3c(c(C(F)(F)F)c2)B(O)OC3)c2oc(C(C)C)c(C)c(=O)c2c1]

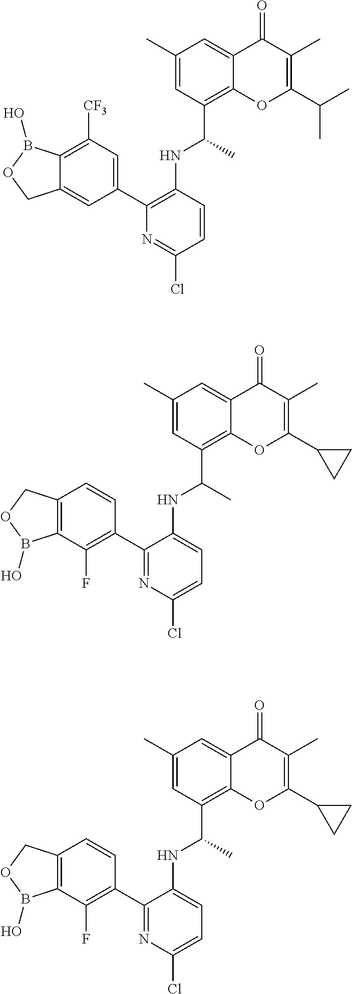 [SMILES: Cc1cc([C@H](C)Nc2ccc(Cl)nc2-c2cc3c(c(C(F)(F)F)c2)B(O)OC3)c2oc(C(C)C)c(C)c(=O)c2c1 ; Cc1cc(C(C)Nc2ccc(Cl)nc2-c2ccc3c(c2F)B(O)OC3)c2oc(C3CC3)c(C)c(=O)c2c1 ; Cc1cc([C@H](C)Nc2ccc(Cl)nc2-c2ccc3c(c2F)B(O)OC3)c2oc(C3CC3)c(C)c(=O)c2c1]

or a tautomer, enantiomer, diastereomer, mixture, salt, hydrate, solvate, or deuterated form thereof.

One aspect of the present disclosure includes wherein the compound is a racemate.

One aspect of the present disclosure includes wherein the compound is a single stereoisomer substantially free of any alternative forms.

One aspect of the present disclosure includes wherein the stereoisomeric form is R.

One aspect of the present disclosure includes wherein the compound is a tautomeric form of a preferred equilibria.

One embodiment of the present disclosure includes a pharmaceutical composition comprising a therapeutically effective amount of a compound of the present disclosure and a pharmaceutically acceptable excipient.

One embodiment of the present disclosure includes a method of inhibiting cell proliferation comprising contacting a cell with an effective amount of a compound of the present disclosure.

One embodiment of the present disclosure includes a method for treating cancer in a patient comprising administering a therapeutically effective amount of a compound of the present disclosure to a patient in need thereof.

One embodiment of the present disclosure includes a method of treating a PI3K-mediated disease or disorder in a patient in need of such treatment, the method comprising administering to the patient a therapeutically effective amount of a compound of the present disclosure.

One aspect includes a method of treating a disease or disorder mediated by one or more PIK3CA genes comprising modulating one or more of wild type or one or more mutations of the one or more PIK3CA genes. The scope of the present disclosure includes all other isoforms.

One aspect includes modulating one mutation.

One aspect includes modulating two or more mutations.

One aspect includes modulating wild type.

One aspect includes wherein the PIK3CA is a PIK3CA mutant.

One aspect includes wherein the PIK3CA mediates a cancer.

One aspect includes wherein the PIK3CA regulates cancer initiation, progression, or metasasis.

One aspect includes wherein wherein the one or more mutations are any p110 mutation.

One aspect includes wherein the one or more mutations are selected from one or more mutations of H1047, E545, E542, N345, E726, C420, Q546, G118, E453, Q546, G1049, M1043, K111, E81, N1044, and E110.

One aspect includes wherein the one or more mutations are selected from one or more mutations of H1047, E545, and E542.

One aspect includes wherein modulation is inhibition.

One aspect includes wherein modulation is selective inhibiton for one or more mutations over wild-type.

One aspect includes wherein the mutations are selected from H1047X, E545X, and E542X.

One aspect includes wherein the mutation is H1047X.

One aspect includes wherein the mutation is H1047L

One aspect includes wherein the mutation is H1047R.

One aspect includes wherein a mutation is E545X.

One aspect includes wherein the mutation is E545K.

One aspect includes wherein a mutation is E542X.

One aspect includes wherein the mutation is E542K.

One aspect includes wherein the method further comprises the compound chemically capturing a solvent accessible surface area of a protein.

One aspect includes wherein the solvent accessible surface area comprises one or more amino acid residue.

One aspect includes wherein the amino acid residue is selected from one or more of arginine, histidine, lysine, glutamic acid, serine, threonine, or glutamine.

One aspect includes wherein the amino acid residue is histidine.

One aspect includes wherein the histidine is HIS1048.

One aspect includes wherein chemical capture is interacting.

One aspect includes wherein interacting is binding.

One aspect includes wherein binding is covalent binding.

One aspect includes wherein binding is hydrogen bonding.

One aspect includes wherein the method further comprises administering a compound of the present disclosure.

One embodiment of the present disclosure includes a method for inhibiting PI3K activity in a cell, comprising modulating a solvent accessible surface area of a protein with a compound of the present disclosure.

One aspect includes wherein the modulating is in vitro.

One aspect includes wherein the modulating is in vivo.

One aspect includes wherein the PI3K targeted gene is PIK3CA.

One aspect includes wherein the PI3K is PI3Kα.

One aspect includes wherein the PI3Kα is a PI3Kα mutant.

One aspect includes wherein the PI3Kα is a PI3Kα wild type.

One aspect includes wherein the PI3K mediates a cancer.

One aspect includes wherein the PI3K regulates cancer initiation, progression, or metastasis.

One aspect includes wherein the one or more mutations are selected from one or more mutations of H1047, E545, E542, N345, E726, C420, Q546, G118, E453, Q546, G1049, M1043, K111, E81, N1044, and E110.

One aspect includes wherein the one or more mutations are selected from one or more mutations of of H1047, E545, and E542.

One aspect includes wherein modulation is inhibition.

One aspect includes wherein modulation is selective inhibition over wild-type, providing preferential inhibition at a multiple level of: greater than 1, between about 1.5 to about 20 or greater, between about 1.5 and 100 or greater.

One aspect includes wherein the mutations are selected from H1047X, E545X, and E542X.

One aspect includes wherein a mutation is H1047X.

One aspect includes wherein the mutation is H1047L

One aspect includes wherein the mutation is H1047R.

One aspect includes wherein the mutation is GLU545X.

One aspect includes wherein the mutation is E545K.

One aspect includes wherein the mutation is GLU542X.

One aspect includes wherein the mutation is E542K.

One aspect includes further comprising the compound chemically capturing a solvent accessible surface area of the protein.

One aspect includes wherein the solvent accessible surface area comprises one or more amino acid residue.

One aspect includes wherein the amino acid residue is selected from one or more of arginine, histidine, lysine, glutamic acid, serine, threonine, or glutamine.

One aspect includes wherein the amino acid residue is histidine.

One aspect includes wherein the histidine is H1048.

One aspect includes wherein chemical capture is interacting.

One aspect includes wherein interacting is binding.

One aspect includes wherein binding is covalent binding.

One aspect includes wherein binding is hydrogen bonding.

One embodiment of the present disclosure includes a method of inhibiting PI3K comprising chemically capturing a solvent accessible surface area and modulating a p110 mutated protein subunit.

One aspect includes wherein the solvent accessible surface area comprises one or more amino acid residue.

One aspect includes wherein the amino acid residue is selected from one or more of arginine, histidine, lysine, glutamic acid, serine, threonine, or glutamine.

One aspect includes wherein the amino acid residue is histidine.

One aspect includes wherein the histidine is H1048.

One aspect includes wherein chemical capture is interacting.

One aspect includes wherein interacting is binding.

One aspect includes wherein binding is covalent binding.

One aspect includes wherein binding is hydrogen bonding.

One aspect includes wherein the p110 mutated protein subunit comprises at least one amino acid mutation compared to the wild type p110 protein subunit.

One aspect includes wherein the at least one amino acid mutation are selected from one or more mutations of H1047, E545, E542, N345, E726, C420, Q546, G118, E453, Q546, G1049, M1043, K111, E81, N1044, and E110.

One aspect includes wherein the one or more mutations are selected from one or more of H1047, E545, and E542.

One aspect includes comprising administering a compound of the present disclosure.

One embodiment of the present diclsoure includes a method of treating a disease or disorder mediated by PI3KA, comprising chemically capturing a solvent accessible amino acid residue and modulating one or more of H1047X, E545X, and E542X.

One aspect includes wherein the amino acid residue is selected from one or more of arginine, histidine, lysine, glutamic acid, serine, threonine, or glutamine.

One aspect includes wherein the amino acid residue is histidine.

One aspect includes wherein the histidine is HIS1048.

One aspect includes wherein chemical capture is interacting.

One aspect includes wherein interacting is binding.

One aspect includes wherein binding is covalent binding.

One aspect includes wherein binding is hydrogen bonding.

One aspect includes modulating one or more of H1047L and H1047R.

One aspect includes modulating GLU545K.

One aspect includes modulating GLU542K.

One aspect includes administering a compound of the present disclosure

One embodiment of the present disclosure includes a method of inhibiting a PIK3CA gene target protein (PI3K) comprising modulating two or more mutant variants selected from mutations of H1047, E545, E542, N345, E726, C420, Q546, G118, E453, Q546, G1049, M1043, K111, E81, N1044, and E110.

One aspect includes wherein the two or more mutant variants are selected from mutations of H1047, E545, and E542

One embodiment of the present disclosure includes a method of treating a disease or disorder mediated by PIK3CA, comprising modulating two or more mutant variants selected from mutations of H1047, E545, E542, N345, E726, C420, Q546, G118, E453, Q546, G1049, M1043, K111, E81, N1044, and E110.

One aspect includes wherein the two or more mutant variants are selected from mutations of H1047, E545, and E542.

One aspect includes administering a compound of the present disclosure.

One embodiment of the present disclosure includes a method of modulating a PI3K to treat a disease or disorder by interacting a compound of the present disclosure with at least two mutant variants.

One aspect includes wherein the PI3K gene target is PIK3CA.

One aspect includes wherein the PI3K mediates a cancer.

One aspect includes wherein the PI3K regulates cancer initiation, progression, or metastasis.

One aspect includes wherein the mutant variants are selected from mutations of H1047, E545, E542, N345, E726, C420, Q546, G118, E453, Q546, G1049, M1043, K111, E81, N1044, and E110.

One aspect includes wherein the mutant variants are selected from mutations of one or more of H1047, E545, and E542.

One aspect includes wherein modulation is inhibition.

One aspect includes wherein modulation is selective inhibition over wild-type.

One aspect includes wherein the mutation is H1047X.

One aspect includes wherein the mutation is H1047L.

One aspect includes wherein the mutation is H1047R.

One aspect includes wherein the mutation is E545X.

One aspect includes wherein the mutation is E545K.

One aspect includes wherein the mutation is E542.

One aspect includes wherein the mutation is E542K.

One aspect includes administering a compound of the present disclosure and capturing a solvent accessible surface area of a protein.

One aspect includes wherein the solvent accessible surface area comprises one or more amino acid residue.

One aspect includes wherein the amino acid residue is selected from one or more of arginine, histidine, lysine, glutamic acid, serine, threonine, or glutamine.

One aspect includes wherein the amino acid residue is histidine.

One aspect includes wherein the histidine is HIS1048.

One aspect includes wherein chemical capture is interacting.

One aspect includes wherein interacting is binding.

One aspect includes wherein binding is covalent binding.

One aspect includes wherein binding is hydrogen binding.

In one embodiment, the present disclosure includes a method of any one of claims 156 to 273, wherein the disease or disorder is cancer.

One aspect includes wherein the disease or disorder is PROS: PIK3CA-Related Overgrowth Spectrum.

One aspect includes wherein the disease or disorder is breast cancer, colorectal cancer, uterine cancer, bladder cancer, lung cancer, glioma, head and neck cancer, or other solid tumors.

One aspect includes wherein the disease or disorder is breast cancer.

In one embodiment, the present disclosure includes a method comprising administration of one or more additional therapeutic agent.

One aspect includes wherein administration is of two or more additional therapeutic agents.

One aspect includes wherein the additional therapeutic agents are selected from selective estrogen receptor degraders, Protac-mediated estrogen receptor inhibitors, complete estrogen receptor antagonists, sarcoplasmic reticulum calcium ATPase inhibitors, CDK2/4/6 inhibitors, CDK4/6 inhibitors, and aromatase inhibitors.

One aspect includes wherein the additional therapeutic agents are selected from fulvestrant, vepdegestrant, palazestrant, imlunestrant, elacestrant, giredestrant, camizestrant, palbociclib, ribociclib, abemaciclib, anastronzole, exemestane, and letrozole.

One aspect includes wherein each agent is provided in a separate dosage form.

One aspect includes wherein one or more agent is provided in a combined dosage form.

In one embodiment, the present disclosure includes a method to modulate one or more PI3K enzymes to regulate one or more of disease initiation and progression comprising interaction with at least one histidine and modulation of at least one surface accessible amino acid or residue.

One aspect includes wherein one or more PI3K is inhibited.

One aspect includes wherein the PI3K is PI3Kα.

One aspect includes wherein the PI3Kα is a mutated variant thereof.

In one embodiment, the present disclosure includes a method for treating cancer in a patient in need thereof, comprising:
determining that the cancer is associated with a PI3K wild-type or one or more PI3K mutations; and
administering to the patient a therapeutically effective amount of a compound of the present disclosure

One aspect includes wherein the PI3K is a mutated variant thereof.

In one embodiment, the present disclosure includes a compound of the present disclosure, for use in therapy.

In one embodiment, the present disclosure includes a compound of the present disclosure, for use in the treatment of cancer.

In one embodiment, the present disclosure includes a compound of the present disclosure, for use in the inhibition of PI3K.

In one aspect, the PI3K is PI3Kα.

In one aspect, the PI3Kα is wild type.

In one aspect, the PI3Kα is a mutated variant thereof.

In one embodiment, the present disclosure includes a use of a compound of the present disclosure, in the manufacture of a medicament for the treatment of cancer.

In one embodiment, the present disclosure includes a use of a compound of the present disclosure, in the manufacture of a medicament for the inhibition of activity of PI3K.

In one embodiment, the present disclosure includes a use of a compound of the present disclosure in the manufacture of a medicament for the treatment of a PI3K-mediated disease or disorder.

In one aspect, the PI3K is PI3Kα.

In one aspect, the PI3Kα is wild type.

In one aspect, the PI3Kα is a mutated variant thereof.

One embodiment of the present disclosure includes a process for preparing a compound of the present disclosure.

One embodiment of the present disclosure includes a compound obtained by a process of the present disclosure.

One embodiment of the present disclosure includes an adduct comprising a compound having a warhead capable of chemical capture of a solvent accessible surface area (SASA) of a protein. In one embodiment, the SASA comprises a residue of one or more of an available arginine, histidine, lysine, glutamic acid, serine, threonine, and glutamine. In one embodiment, the SASA comprises a histidine residue. In one embodiment, the chemical capture is interaction. In one embodiment, the interaction is binding. In one embodiment, the binding is covalent binding. In one embodiment, the binding is hydrogen bonding. In one embodiment, the warhead comprises at least one boron heteroatom. In one embodiment, the warhead comprises a ring or ring system having at least one boron atom incorporated therein. In one embodiment, the warhead comprises at least one B(OH)₂ group as a substituent. In one embodiment, the warhead does not include a boron heteroatom. In one embodiment, the warhead is selected from: a cyclohexenone derivative, an alkyl halide derivative, a sulfonyl derivative, an α-cyanoenone derivative, and an epoxide or spiro-epoxide derivative.

In one embodiment, the present disclosure includes a compound of formula XX:

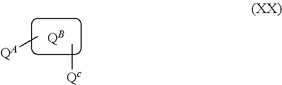

or a tautomer, enantiomer, diastereomer, mixture, salt, hydrate, solvate, or deuterated form thereof,
wherein
   Q^{B} comprises an optionally substituted 8- to 14-membered spiro or fused ring system optionally containing one or more heteroatoms selected from the group consisting of O, N, or S;
   QA is ^{XX}A1-^{XX}L¹-^{XX}L²-C(^{XX}R^{1a}-^{XX}R^{1b}), wherein:
      ^{XX}A1 is a warhead moiety that provides chemical capture of an available solvent accessible surface area of a protein;
      ^{XX}L¹ is selected from the group consisting of a direct bond, optionally substituted arylene, and optionally substituted heteroarylene;
      ^{XX}L² is selected from the group consisting of O and NH; and
      each of ^{XX}R^{1a} and ^{XX}R^{1b} independently is selected from the group consisting of H, halogen, CN, C₁₋₆ alkyl, C₂₋₆ alkenyl, C₂₋₆ alkynyl, C₁₋₆ haloalkyl, and C₁₋₆ alkoxy;
      or ^{XX}L²-C(^{XX}R^{1a})(^{XX}R^{1b}) combines to form NHC(O), C(O)NH, NHS(O)₂, S(O)₂NH, or C(NH)NH₂;
   Q^{C} is ^{XX}Y(^{XX}R^{3a}-^{XX}R^{3b}) wherein:
   ^{XX}Y is H, C^{XX}R^{3c}, N, O, or S;
   when ^{XX}Y is H, each of ^{XX}R^{3a} and ^{XX}R^{3b} is absent;
   when ^{XX}Y is N,
      each of ^{XX}R^{3a} and ^{XX}R^{3b} independently is selected from the group consisting of H, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl; or
      Y, as an N atom, combines with ^{XX}R^{3a} and ^{XX}R^{3b} to form a heterocyclic or heteroaromatic mono ring or spiro or fused ring system, which may contain one or more additional heteroatoms selected from the group consisting of N, O, and S, and wherein the ring or ring system is optionally substituted;
   when Y is C^{XX}R^{3c},
      ^{XX}R^{3c} is absent or is selected from the group consisting of hydrogen, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl;
      each of ^{XX}R^{3a} and ^{XX}R^{3b} independently is selected from the group consisting of H, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl; or
      ^{XX}Y as an C atom, combines with ^{XX}R^{3a} and ^{XX}R^{3b} to form a cycloalkyl, aryl, heterocyclic, or heteroaromatic mono ring or spiro or fused ring system, where the heterocyclic and heteroaromatic rings contain one or more heteroatoms selected from the group consisting of N, O, and S, and wherein the ^{XX}Y-containing ring or ring system is optionally substituted; or
   when ^{XX}Y is O or S, ^{XX}R^{3a} is absent and ^{XX}R^{3b} is selected from the group consisting of H, C₁₋₆ alkyl, C₂₋₆ alkenyl, and C₂₋₆ alkynyl.

One embodiment of the present disclosure includes a compound of Table 1, or a tautomer, enantiomer, diastereomer, mixture, salt, hydrate, solvate, or deuterated form thereof.

TABLE 1
Compounds of the present disclosure

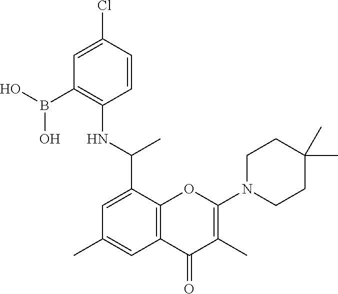 [SMILES: Cc1cc(C(C)Nc2ccc(Cl)cc2B(O)O)c2oc(N3CCC(C)(C)CC3)c(C)c(=O)c2c1]

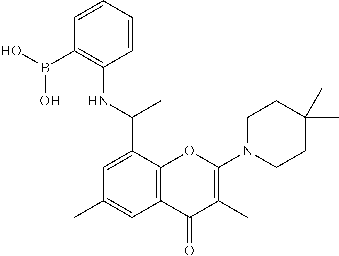 [SMILES: Cc1cc(C(C)Nc2ccccc2B(O)O)c2oc(N3CCC(C)(C)CC3)c(C)c(=O)c2c1]

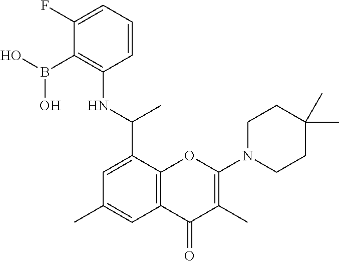 [SMILES: Cc1cc(C(C)Nc2cccc(F)c2B(O)O)c2oc(N3CCC(C)(C)CC3)c(C)c(=O)c2c1]

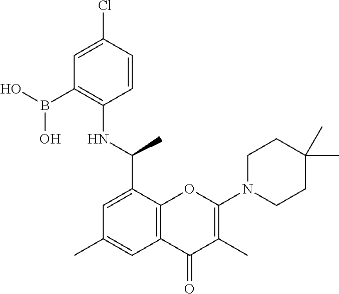 [SMILES: Cc1cc([C@H](C)Nc2ccc(Cl)cc2B(O)O)c2oc(N3CCC(C)(C)CC3)c(C)c(=O)c2c1]

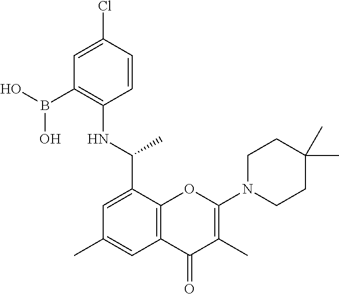 [SMILES: Cc1cc([C@@H](C)Nc2ccc(Cl)cc2B(O)O)c2oc(N3CCC(C)(C)CC3)c(C)c(=O)c2c1]

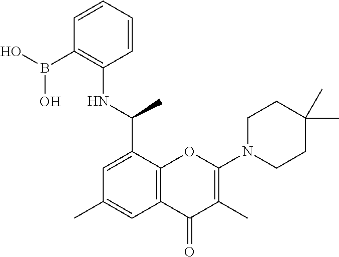 [SMILES: Cc1cc([C@H](C)Nc2ccccc2B(O)O)c2oc(N3CCC(C)(C)CC3)c(C)c(=O)c2c1]

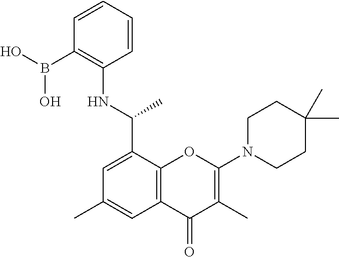 [SMILES: Cc1cc([C@@H](C)Nc2ccccc2B(O)O)c2oc(N3CCC(C)(C)CC3)c(C)c(=O)c2c1]

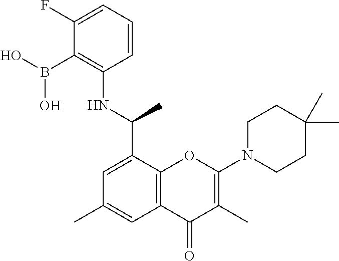 [SMILES: Cc1cc([C@H](C)Nc2cccc(F)c2B(O)O)c2oc(N3CCC(C)(C)CC3)c(C)c(=O)c2c1]

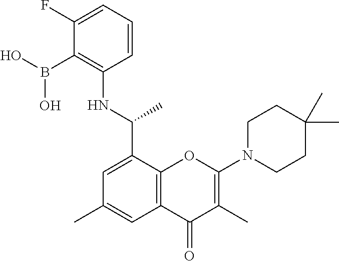 [SMILES: Cc1cc([C@@H](C)Nc2cccc(F)c2B(O)O)c2oc(N3CCC(C)(C)CC3)c(C)c(=O)c2c1]

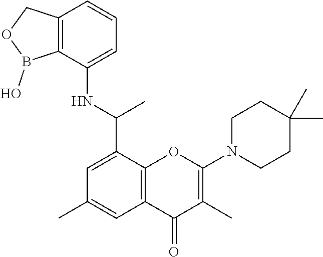 [SMILES: Cc1cc(C(C)Nc2cccc3c2B(O)OC3)c2oc(N3CCC(C)(C)CC3)c(C)c(=O)c2c1]

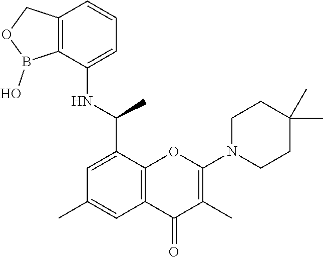 [SMILES: Cc1cc([C@H](C)Nc2cccc3c2B(O)OC3)c2oc(N3CCC(C)(C)CC3)c(C)c(=O)c2c1]

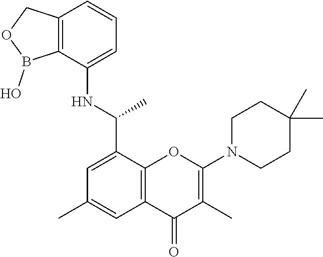 [SMILES: Cc1cc([C@@H](C)Nc2cccc3c2B(O)OC3)c2oc(N3CCC(C)(C)CC3)c(C)c(=O)c2c1]

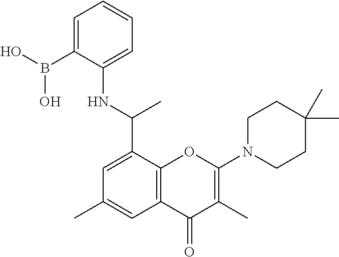 [SMILES: Cc1cc(C(C)Nc2ccccc2B(O)O)c2oc(N3CCC(C)(C)CC3)c(C)c(=O)c2c1]

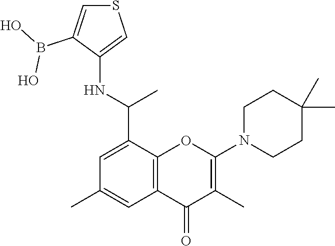 [SMILES: Cc1cc(C(C)Nc2cscc2B(O)O)c2oc(N3CCC(C)(C)CC3)c(C)c(=O)c2c1]

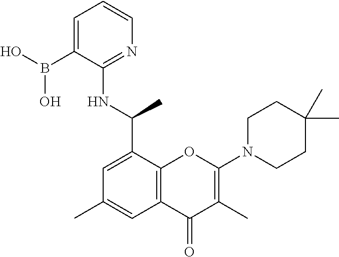 [SMILES: Cc1cc([C@H](C)Nc2ncccc2B(O)O)c2oc(N3CCC(C)(C)CC3)c(C)c(=O)c2c1]

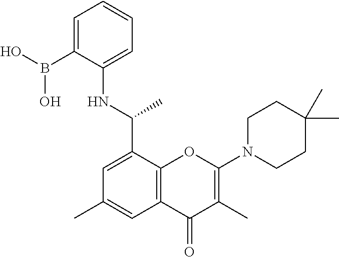 [SMILES: Cc1cc([C@@H](C)Nc2ccccc2B(O)O)c2oc(N3CCC(C)(C)CC3)c(C)c(=O)c2c1]

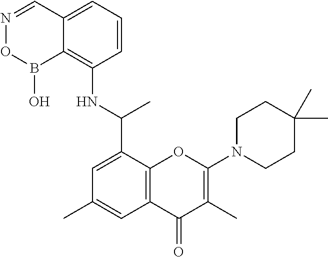 [SMILES: Cc1cc(C(C)Nc2cccc3c2B(O)ON=C3)c2oc(N3CCC(C)(C)CC3)c(C)c(=O)c2c1]

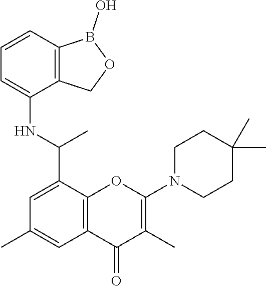

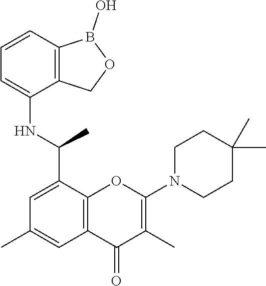 [SMILES: Cc1cc([C@H](C)Nc2cccc3c2COB3O)c2oc(N3CCC(C)(C)CC3)c(C)c(=O)c2c1]

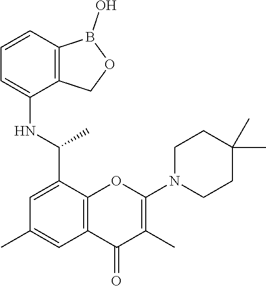

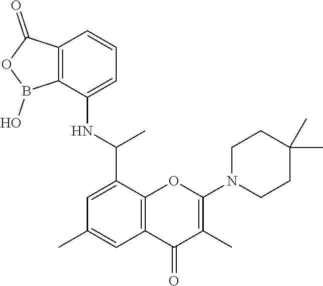 [SMILES: Cc1cc(C(C)Nc2cccc3c2B(O)OC3=O)c2oc(N3CCC(C)(C)CC3)c(C)c(=O)c2c1]

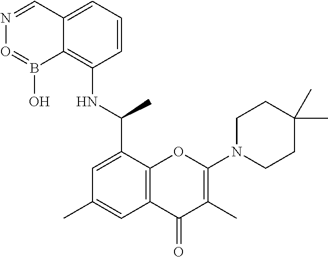

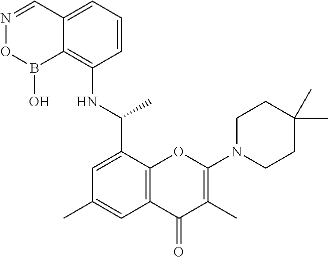 [SMILES: Cc1cc([C@@H](C)Nc2cccc3c2B(O)ON=C3)c2oc(N3CCC(C)(C)CC3)c(C)c(=O)c2c1]

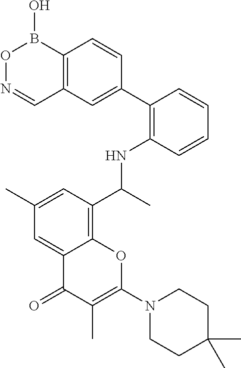 [SMILES: Cc1cc(C(C)Nc2ccccc2-c2ccc3c(c2)C=NOB3O)c2oc(N3CCC(C)(C)CC3)c(C)c(=O)c2c1]

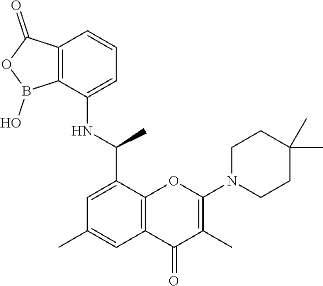 [SMILES: Cc1cc([C@H](C)Nc2cccc3c2B(O)OC3=O)c2oc(N3CCC(C)(C)CC3)c(C)c(=O)c2c1]

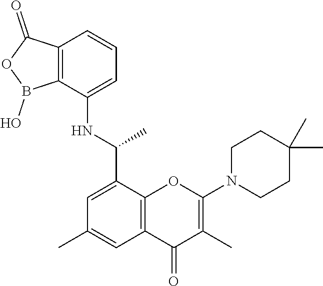 [SMILES: Cc1cc([C@@H](C)Nc2cccc3c2B(O)OC3=O)c2oc(N3CCC(C)(C)CC3)c(C)c(=O)c2c1]

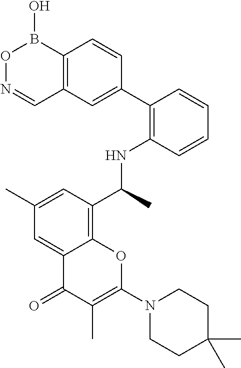 [SMILES: Cc1cc([C@H](C)Nc2ccccc2-c2ccc3c(c2)C=NOB3O)c2oc(N3CCC(C)(C)CC3)c(C)c(=O)c2c1]

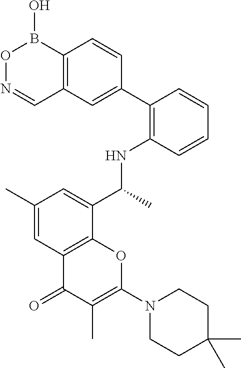 [SMILES: Cc1cc([C@@H](C)Nc2ccccc2-c2ccc3c(c2)C=NOB3O)c2oc(N3CCC(C)(C)CC3)c(C)c(=O)c2c1]

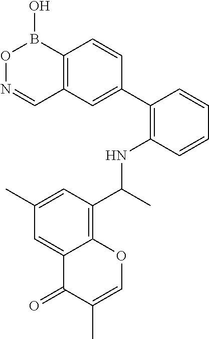 [SMILES: Cc1cc(C(C)Nc2ccccc2-c2ccc3c(c2)C=NOB3O)c2occ(C)c(=O)c2c1]

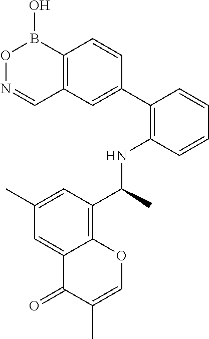 [SMILES: Cc1cc([C@H](C)Nc2ccccc2-c2ccc3c(c2)C=NOB3O)c2occ(C)c(=O)c2c1]

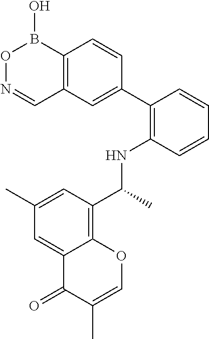 [SMILES: Cc1cc([C@@H](C)Nc2ccccc2-c2ccc3c(c2)C=NOB3O)c2occ(C)c(=O)c2c1]

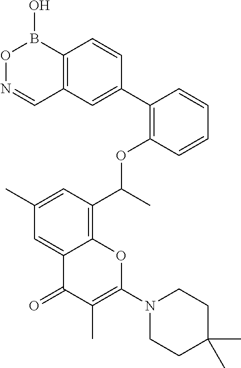 [SMILES: Cc1cc(C(C)Oc2ccccc2-c2ccc3c(c2)C=NOB3O)c2oc(N3CCC(C)(C)CC3)c(C)c(=O)c2c1]

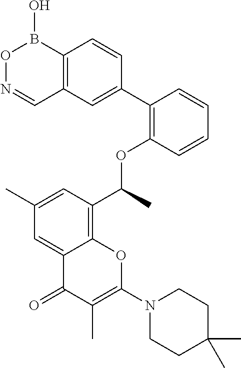 [SMILES: Cc1cc([C@H](C)Oc2ccccc2-c2ccc3c(c2)C=NOB3O)c2oc(N3CCC(C)(C)CC3)c(C)c(=O)c2c1]

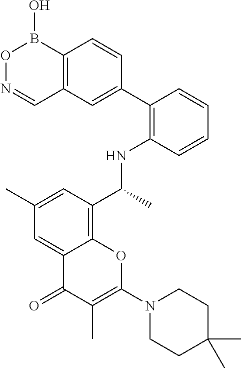 [SMILES: Cc1cc([C@@H](C)Nc2ccccc2-c2ccc3c(c2)C=NOB3O)c2oc(N3CCC(C)(C)CC3)c(C)c(=O)c2c1]

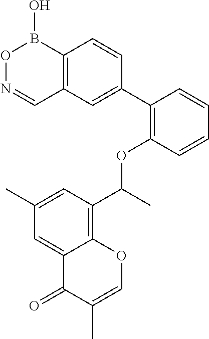 [SMILES: Cc1cc(C(C)Oc2ccccc2-c2ccc3c(c2)C=NOB3O)c2occ(C)c(=O)c2c1]

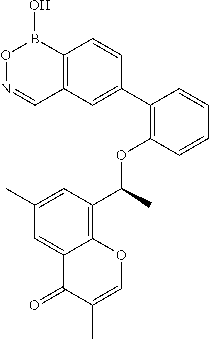 [SMILES: Cc1cc([C@H](C)Oc2ccccc2-c2ccc3c(c2)C=NOB3O)c2occ(C)c(=O)c2c1]

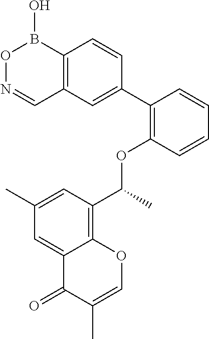 [SMILES: Cc1cc([C@@H](C)Oc2ccccc2-c2ccc3c(c2)C=NOB3O)c2occ(C)c(=O)c2c1]

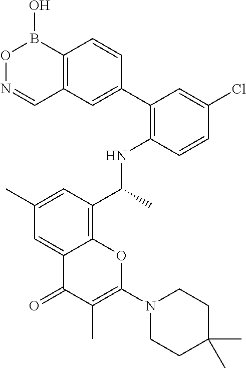 [SMILES: Cc1cc([C@@H](C)Nc2ccc(Cl)cc2-c2ccc3c(c2)C=NOB3O)c2oc(N3CCC(C)(C)CC3)c(C)c(=O)c2c1]

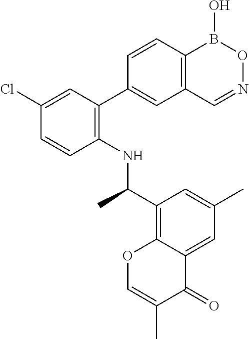 [SMILES: Cc1cc([C@@H](C)Nc2ccc(Cl)cc2-c2ccc3c(c2)C=NOB3O)c2occ(C)c(=O)c2c1]

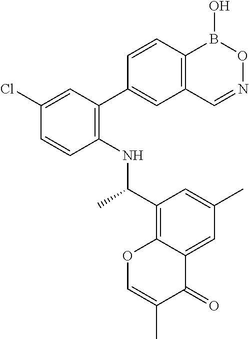 [SMILES: Cc1cc([C@H](C)Nc2ccc(Cl)cc2-c2ccc3c(c2)C=NOB3O)c2occ(C)c(=O)c2c1]

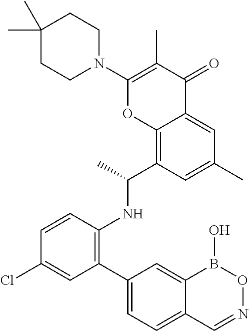

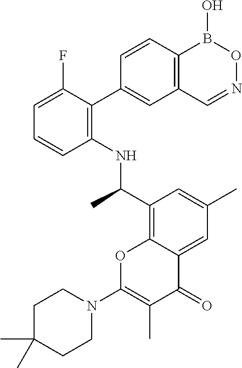

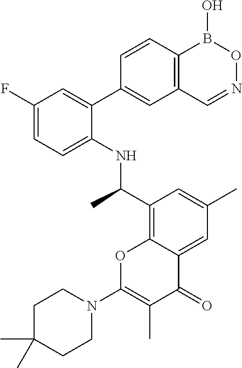

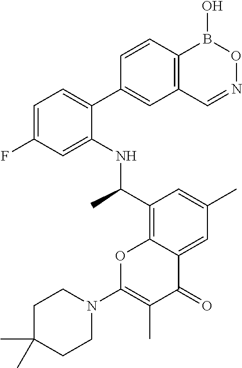

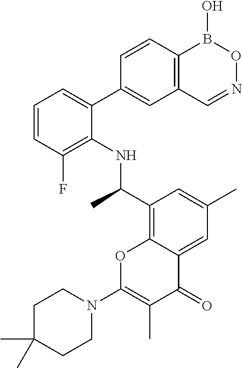

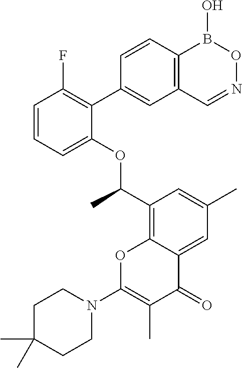

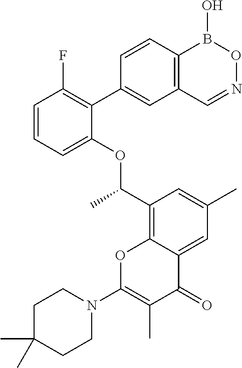

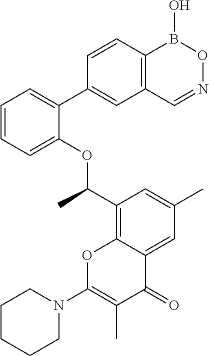 [SMILES: Cc1cc([C@@H](C)Oc2ccccc2-c2ccc3c(c2)C=NOB3O)c2oc(N3CCCCC3)c(C)c(=O)c2c1]

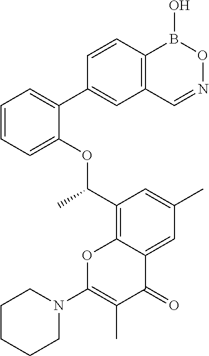 [SMILES: Cc1cc([C@H](C)Oc2ccccc2-c2ccc3c(c2)C=NOB3O)c2oc(N3CCCCC3)c(C)c(=O)c2c1]

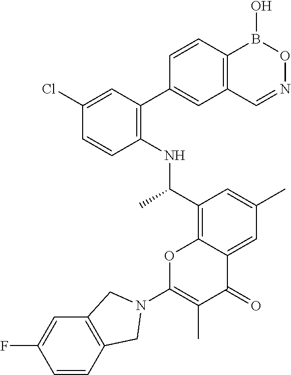 [SMILES: Cc1cc([C@H](C)Nc2ccc(Cl)cc2-c2ccc3c(c2)C=NOB3O)c2oc(N3Cc4ccc(F)cc4C3)c(C)c(=O)c2c1]

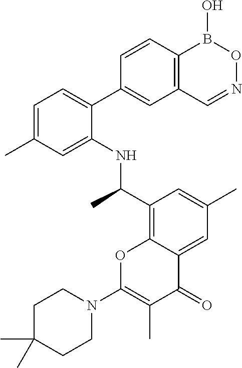

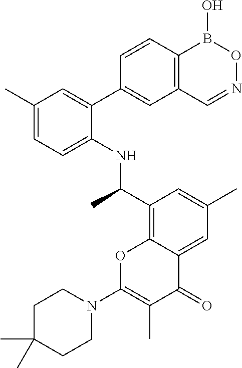

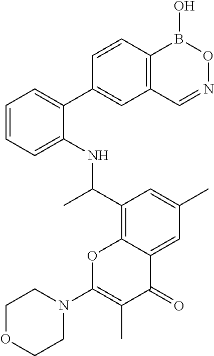 [SMILES: Cc1cc(C(C)Nc2ccccc2-c2ccc3c(c2)C=NOB3O)c2oc(N3CCOCC3)c(C)c(=O)c2c1]

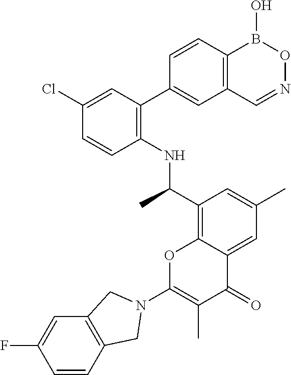 [SMILES: Cc1cc([C@@H](C)Nc2ccc(Cl)cc2-c2ccc3c(c2)C=NOB3O)c2oc(N3Cc4ccc(F)cc4C3)c(C)c(=O)c2c1]

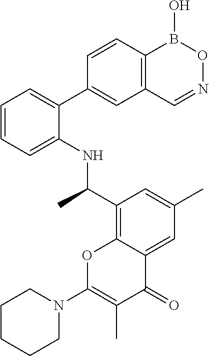 [SMILES: Cc1cc([C@@H](C)Nc2ccccc2-c2ccc3c(c2)C=NOB3O)c2oc(N3CCCCC3)c(C)c(=O)c2c1]

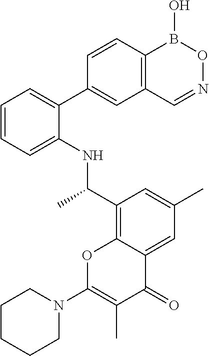 [SMILES: Cc1cc([C@H](C)Nc2ccccc2-c2ccc3c(c2)C=NOB3O)c2oc(N3CCCCC3)c(C)c(=O)c2c1]

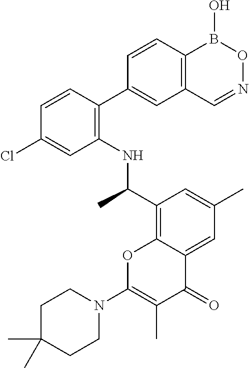

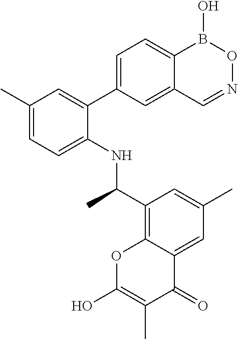 [SMILES: Cc1ccc(N[C@H](C)c2cc(C)cc3c(=O)c(C)c(O)oc23)c(-c2ccc3c(c2)C=NOB3O)c1]

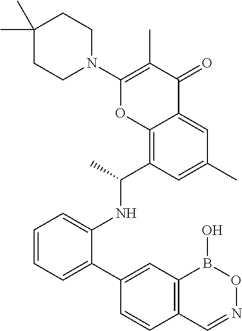

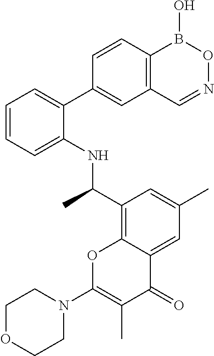 [SMILES: Cc1cc([C@@H](C)Nc2ccccc2-c2ccc3c(c2)C=NOB3O)c2oc(N3CCOCC3)c(C)c(=O)c2c1]

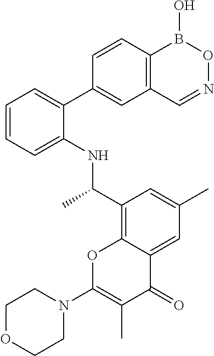 [SMILES: Cc1cc([C@H](C)Nc2ccccc2-c2ccc3c(c2)C=NOB3O)c2oc(N3CCOCC3)c(C)c(=O)c2c1]

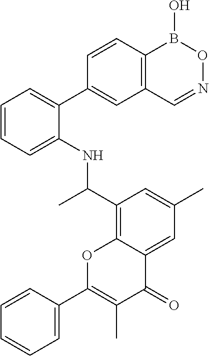 [SMILES: Cc1cc(C(C)Nc2ccccc2-c2ccc3c(c2)C=NOB3O)c2oc(-c3ccccc3)c(C)c(=O)c2c1]

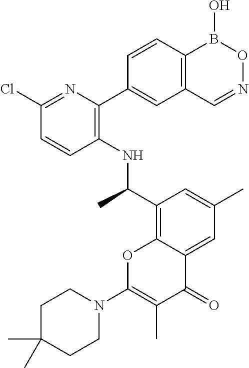

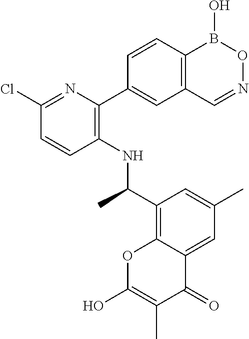 [SMILES: Cc1cc([C@@H](C)Nc2ccc(Cl)nc2-c2ccc3c(c2)C=NOB3O)c2oc(O)c(C)c(=O)c2c1]

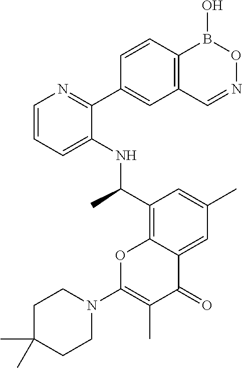

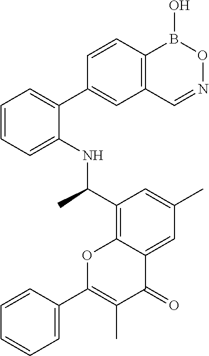 [SMILES: Cc1cc([C@@H](C)Nc2ccccc2-c2ccc3c(c2)C=NOB3O)c2oc(-c3ccccc3)c(C)c(=O)c2c1]

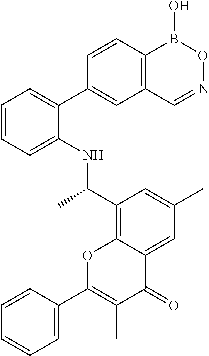 [SMILES: Cc1cc([C@H](C)Nc2ccccc2-c2ccc3c(c2)C=NOB3O)c2oc(-c3ccccc3)c(C)c(=O)c2c1]

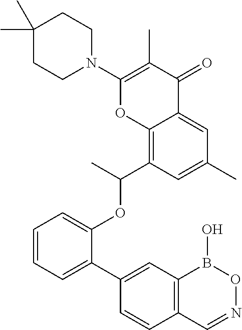

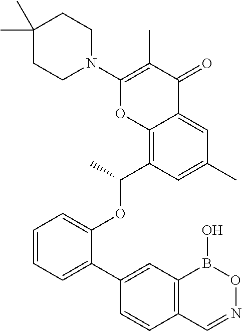

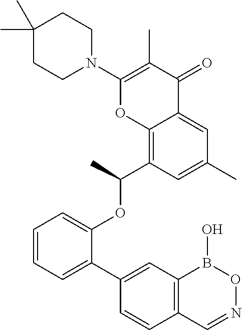

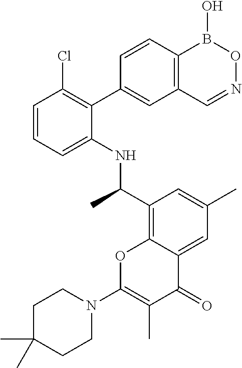

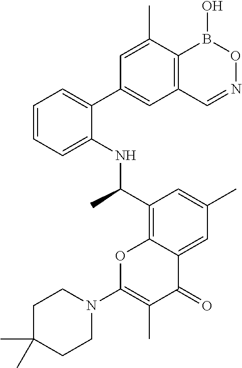

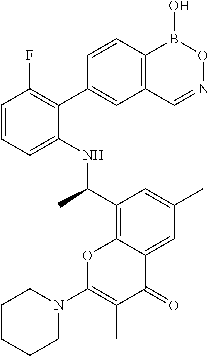 [SMILES: Cc1cc([C@@H](C)Nc2cccc(F)c2-c2ccc3c(c2)C=NOB3O)c2oc(N3CCCCC3)c(C)c(=O)c2c1]

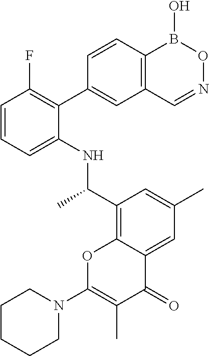 [SMILES: Cc1cc([C@H](C)Nc2cccc(F)c2-c2ccc3c(c2)C=NOB3O)c2oc(N3CCCCC3)c(C)c(=O)c2c1]

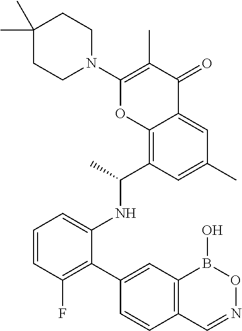

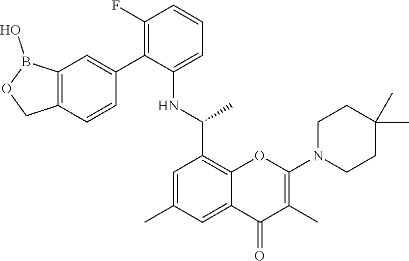 [SMILES: Cc1cc([C@@H](C)Nc2cccc(F)c2-c2ccc3c(c2)B(O)OC3)c2oc(N3CCC(C)(C)CC3)c(C)c(=O)c2c1]

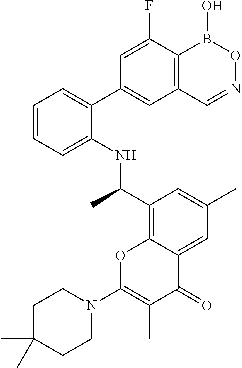

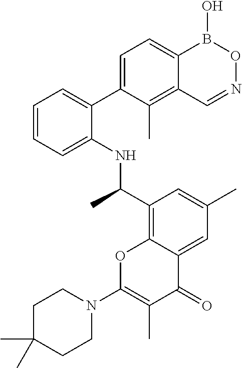

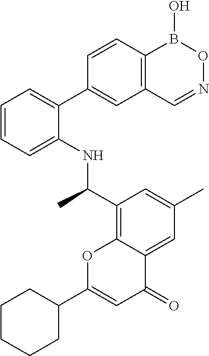 [SMILES: Cc1cc([C@@H](C)Nc2ccccc2-c2ccc3c(c2)C=NOB3O)c2oc(C3CCCCC3)cc(=O)c2c1]

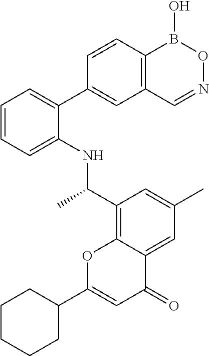 [SMILES: Cc1cc([C@H](C)Nc2ccccc2-c2ccc3c(c2)C=NOB3O)c2oc(C3CCCCC3)cc(=O)c2c1]

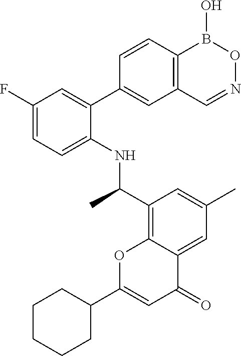 [SMILES: Cc1cc([C@@H](C)Nc2ccc(F)cc2-c2ccc3c(c2)C=NOB3O)c2oc(C3CCCCC3)cc(=O)c2c1]

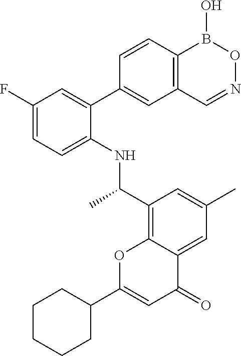 [SMILES: Cc1cc([C@H](C)Nc2ccc(F)cc2-c2ccc3c(c2)C=NOB3O)c2oc(C3CCCCC3)cc(=O)c2c1]

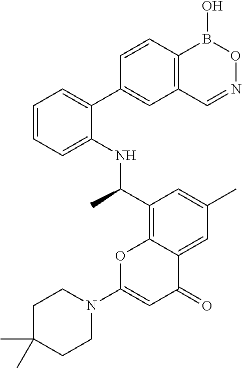

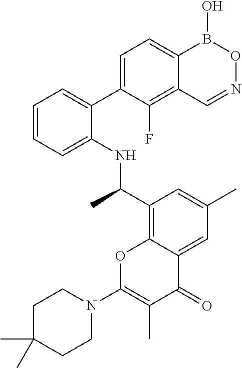

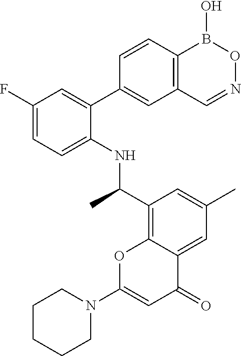 [SMILES: Cc1cc([C@@H](C)Nc2ccc(F)cc2-c2ccc3c(c2)C=NOB3O)c2oc(N3CCCCC3)cc(=O)c2c1]

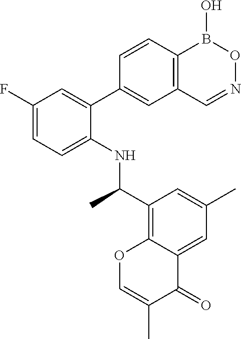 [SMILES: Cc1cc([C@@H](C)Nc2ccc(F)cc2-c2ccc3c(c2)C=NOB3O)c2occ(C)c(=O)c2c1]

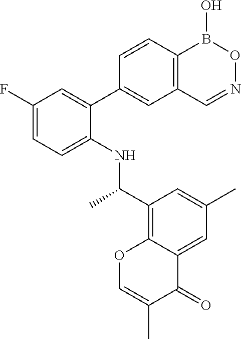 [SMILES: Cc1cc([C@H](C)Nc2ccc(F)cc2-c2ccc3c(c2)C=NOB3O)c2occ(C)c(=O)c2c1]

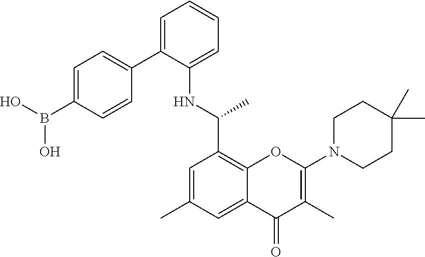 [SMILES: Cc1cc([C@@H](C)Nc2ccccc2-c2ccc(B(O)O)cc2)c2oc(N3CCC(C)(C)CC3)c(C)c(=O)c2c1]

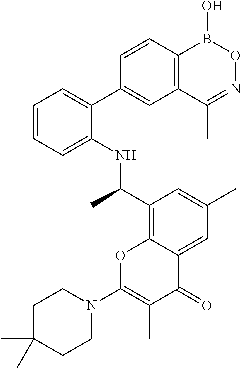

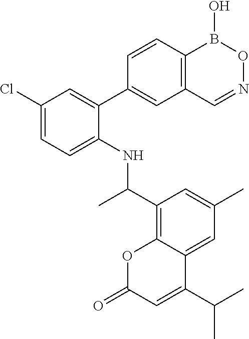 [SMILES: Cc1cc(C(C)Nc2ccc(Cl)cc2-c2ccc3c(c2)C=NOB3O)c2oc(=O)cc(C(C)C)c2c1]

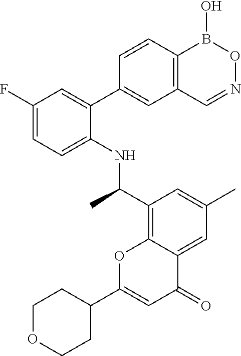 [SMILES: Cc1cc([C@@H](C)Nc2ccc(F)cc2-c2ccc3c(c2)C=NOB3O)c2oc(C3CCOCC3)cc(=O)c2c1]

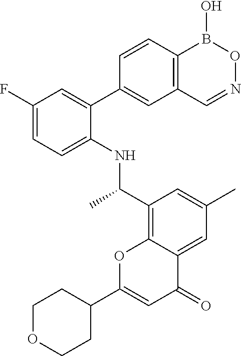 [SMILES: Cc1cc([C@H](C)Nc2ccc(F)cc2-c2ccc3c(c2)C=NOB3O)c2oc(C3CCOCC3)cc(=O)c2c1]

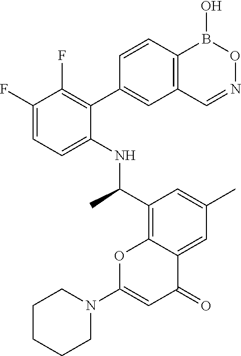 [SMILES: Cc1cc([C@@H](C)Nc2ccc(F)c(F)c2-c2ccc3c(c2)C=NOB3O)c2oc(N3CCCCC3)cc(=O)c2c1]

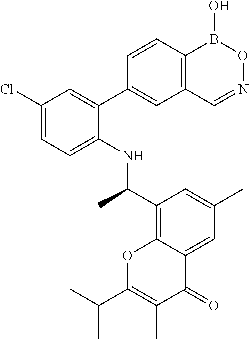 [SMILES: Cc1cc([C@@H](C)Nc2ccc(Cl)cc2-c2ccc3c(c2)C=NOB3O)c2oc(C(C)C)c(C)c(=O)c2c1]

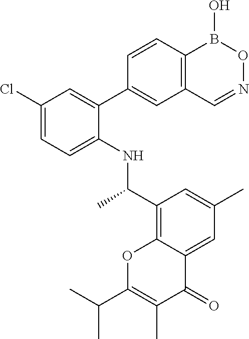 [SMILES: Cc1cc([C@H](C)Nc2ccc(Cl)cc2-c2ccc3c(c2)C=NOB3O)c2oc(C(C)C)c(C)c(=O)c2c1]

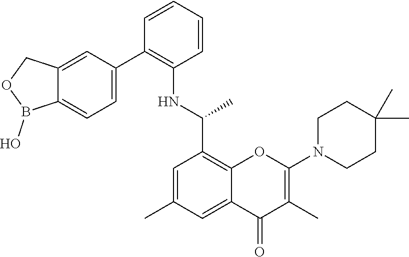 [SMILES: Cc1cc([C@@H](C)Nc2ccccc2-c2ccc3c(c2)COB3O)c2oc(N3CCC(C)(C)CC3)c(C)c(=O)c2c1]

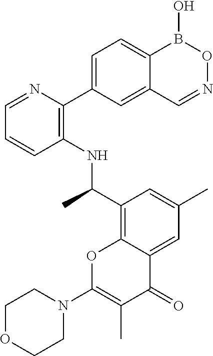 [SMILES: Cc1cc([C@@H](C)Nc2cccnc2-c2ccc3c(c2)C=NOB3O)c2oc(N3CCOCC3)c(C)c(=O)c2c1]

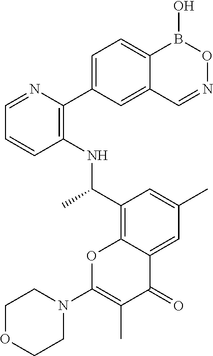 [SMILES: Cc1cc([C@H](C)Nc2cccnc2-c2ccc3c(c2)C=NOB3O)c2oc(N3CCOCC3)c(C)c(=O)c2c1]

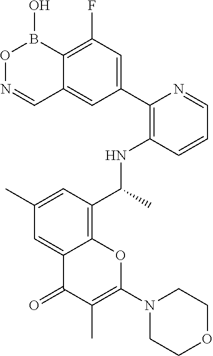 [SMILES: Cc1cc([C@@H](C)Nc2cccnc2-c2cc(F)c3c(c2)C=NOB3O)c2oc(N3CCOCC3)c(C)c(=O)c2c1]

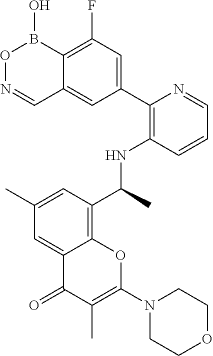 [SMILES: Cc1cc([C@H](C)Nc2cccnc2-c2cc(F)c3c(c2)C=NOB3O)c2oc(N3CCOCC3)c(C)c(=O)c2c1]

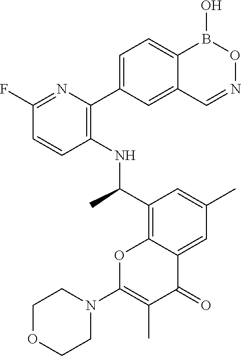 [SMILES: Cc1cc([C@@H](C)Nc2ccc(F)nc2-c2ccc3c(c2)C=NOB3O)c2oc(N3CCOCC3)c(C)c(=O)c2c1]

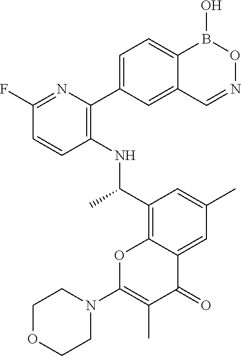 [SMILES: Cc1cc([C@H](C)Nc2ccc(F)nc2-c2ccc3c(c2)C=NOB3O)c2oc(N3CCOCC3)c(C)c(=O)c2c1]

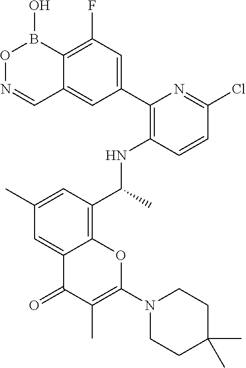 [SMILES: Cc1cc([C@@H](C)Nc2ccc(Cl)nc2-c2cc(F)c3c(c2)C=NOB3O)c2oc(N3CCC(C)(C)CC3)c(C)c(=O)c2c1]

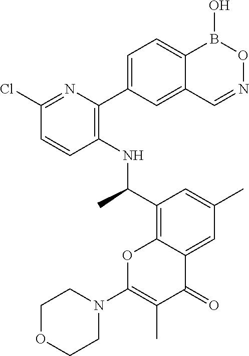 [SMILES: Cc1cc([C@@H](C)Nc2ccc(Cl)nc2-c2ccc3c(c2)C=NOB3O)c2oc(N3CCOCC3)c(C)c(=O)c2c1]

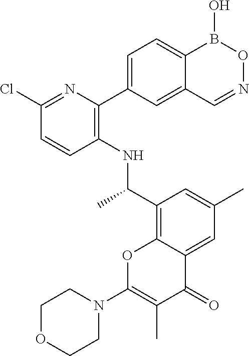 [SMILES: Cc1cc([C@H](C)Nc2ccc(Cl)nc2-c2ccc3c(c2)C=NOB3O)c2oc(N3CCOCC3)c(C)c(=O)c2c1]

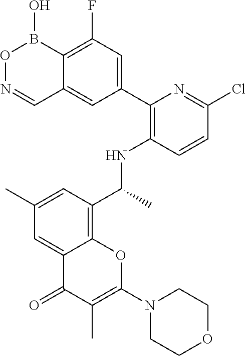 [SMILES: Cc1cc([C@@H](C)Nc2ccc(Cl)nc2-c2cc(F)c3c(c2)C=NOB3O)c2oc(N3CCOCC3)c(C)c(=O)c2c1]

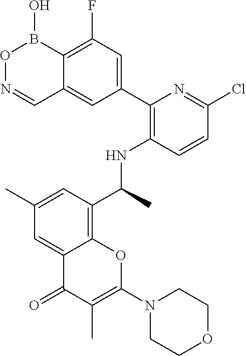 [SMILES: Cc1cc([C@H](C)Nc2ccc(Cl)nc2-c2cc(F)c3c(c2)C=NOB3O)c2oc(N3CCOCC3)c(C)c(=O)c2c1]

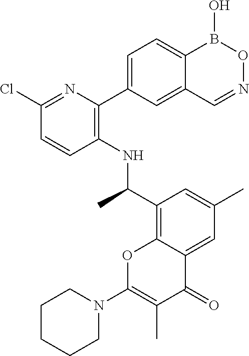 [SMILES: Cc1cc([C@@H](C)Nc2ccc(Cl)nc2-c2ccc3c(c2)C=NOB3O)c2oc(N3CCCCC3)c(C)c(=O)c2c1]

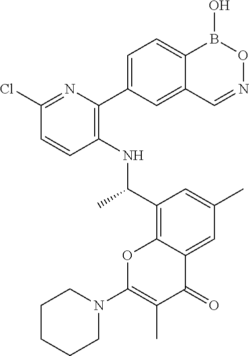 [SMILES: Cc1cc([C@H](C)Nc2ccc(Cl)nc2-c2ccc3c(c2)C=NOB3O)c2oc(N3CCCCC3)c(C)c(=O)c2c1]

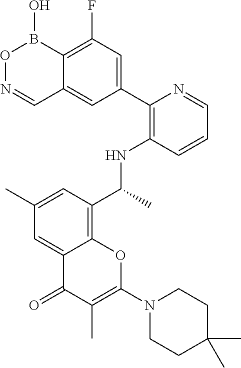 [SMILES: Cc1cc([C@@H](C)Nc2cccnc2-c2cc(F)c3c(c2)C=NOB3O)c2oc(N3CCC(C)(C)CC3)c(C)c(=O)c2c1]

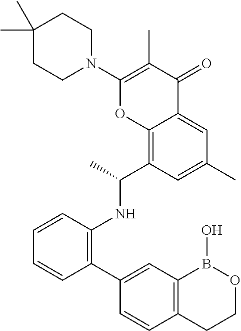

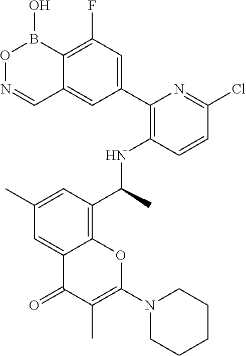 [SMILES: Cc1cc([C@H](C)Nc2ccc(Cl)nc2-c2cc(F)c3c(c2)C=NOB3O)c2oc(N3CCCCC3)c(C)c(=O)c2c1]

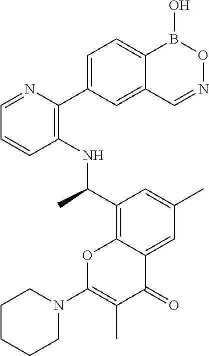 [SMILES: Cc1cc([C@@H](C)Nc2cccnc2-c2ccc3c(c2)C=NOB3O)c2oc(N3CCCCC3)c(C)c(=O)c2c1]

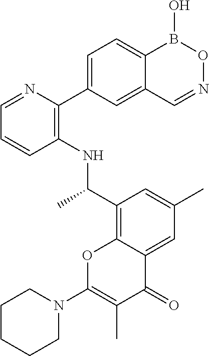 [SMILES: Cc1cc([C@H](C)Nc2cccnc2-c2ccc3c(c2)C=NOB3O)c2oc(N3CCCCC3)c(C)c(=O)c2c1]

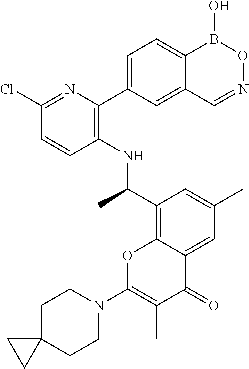 [SMILES: Cc1cc([C@@H](C)Nc2ccc(Cl)nc2-c2ccc3c(c2)C=NOB3O)c2oc(N3CCC4(CC3)CC4)c(C)c(=O)c2c1]

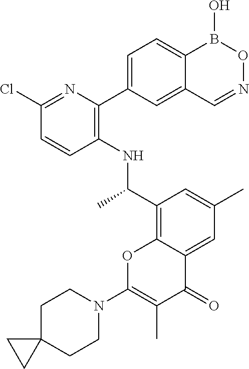 [SMILES: Cc1cc([C@H](C)Nc2ccc(Cl)nc2-c2ccc3c(c2)C=NOB3O)c2oc(N3CCC4(CC3)CC4)c(C)c(=O)c2c1]

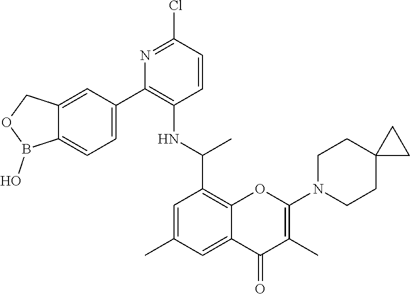 [SMILES: Cc1cc(C(C)Nc2ccc(Cl)nc2-c2ccc3c(c2)COB3O)c2oc(N3CCC4(CC3)CC4)c(C)c(=O)c2c1]

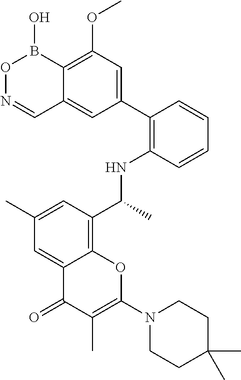 [SMILES: COc1cc(-c2ccccc2N[C@H](C)c2cc(C)cc3c(=O)c(C)c(N4CCC(C)(C)CC4)oc23)cc2c1B(O)ON=C2]

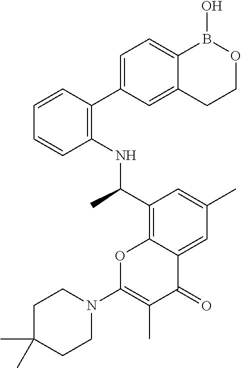

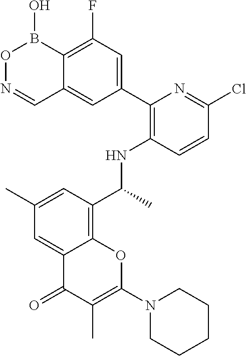 [SMILES: Cc1cc([C@@H](C)Nc2ccc(Cl)nc2-c2cc(F)c3c(c2)C=NOB3O)c2oc(N3CCCCC3)c(C)c(=O)c2c1]

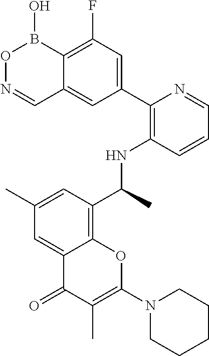 [SMILES: Cc1cc([C@H](C)Nc2cccnc2-c2cc(F)c3c(c2)C=NOB3O)c2oc(N3CCCCC3)c(C)c(=O)c2c1]

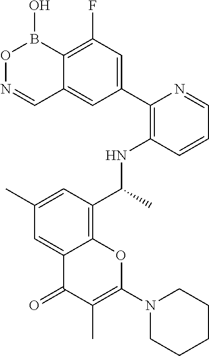 [SMILES: Cc1cc([C@@H](C)Nc2cccnc2-c2cc(F)c3c(c2)C=NOB3O)c2oc(N3CCCCC3)c(C)c(=O)c2c1]

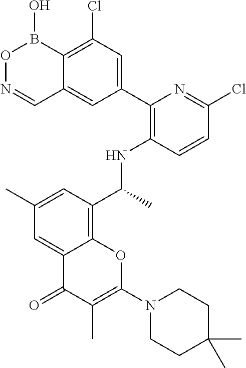 [SMILES: Cc1cc([C@@H](C)Nc2ccc(Cl)nc2-c2cc(Cl)c3c(c2)C=NOB3O)c2oc(N3CCC(C)(C)CC3)c(C)c(=O)c2c1]

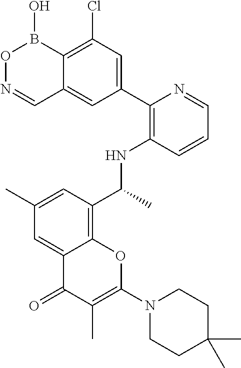 [SMILES: Cc1cc([C@@H](C)Nc2cccnc2-c2cc(Cl)c3c(c2)C=NOB3O)c2oc(N3CCC(C)(C)CC3)c(C)c(=O)c2c1]

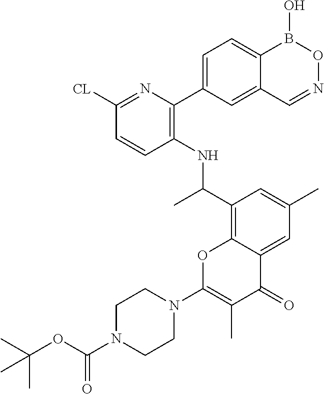

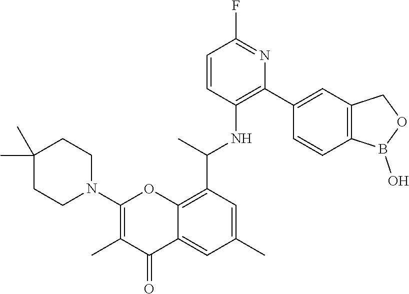 [SMILES: Cc1cc(C(C)Nc2ccc(F)nc2-c2ccc3c(c2)COB3O)c2oc(N3CCC(C)(C)CC3)c(C)c(=O)c2c1]

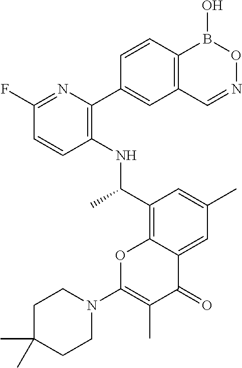

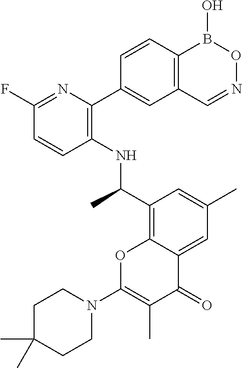

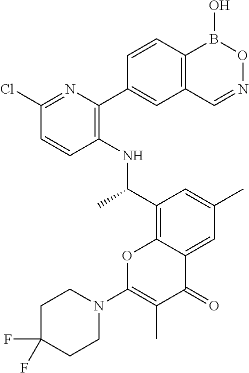 [SMILES: Cc1cc([C@H](C)Nc2ccc(Cl)nc2-c2ccc3c(c2)C=NOB3O)c2oc(N3CCC(F)(F)CC3)c(C)c(=O)c2c1]

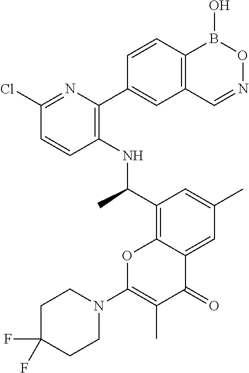 [SMILES: Cc1cc([C@@H](C)Nc2ccc(Cl)nc2-c2ccc3c(c2)C=NOB3O)c2oc(N3CCC(F)(F)CC3)c(C)c(=O)c2c1]

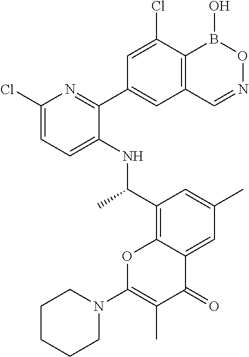 [SMILES: Cc1cc([C@H](C)Nc2ccc(Cl)nc2-c2cc(Cl)c3c(c2)C=NOB3O)c2oc(N3CCCCC3)c(C)c(=O)c2c1]

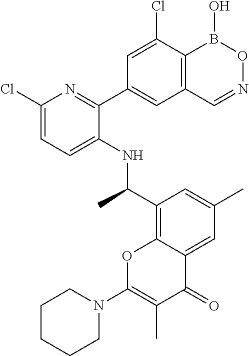 [SMILES: Cc1cc([C@@H](C)Nc2ccc(Cl)nc2-c2cc(Cl)c3c(c2)C=NOB3O)c2oc(N3CCCCC3)c(C)c(=O)c2c1]

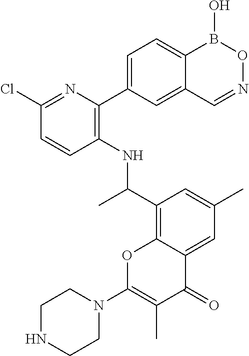 [SMILES: Cc1cc(C(C)Nc2ccc(Cl)nc2-c2ccc3c(c2)C=NOB3O)c2oc(N3CCNCC3)c(C)c(=O)c2c1]

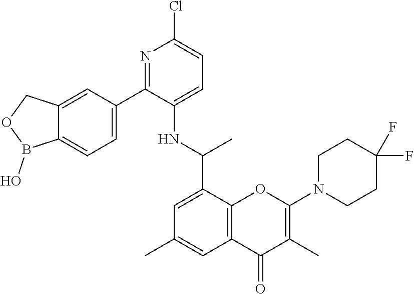 [SMILES: Cc1cc(C(C)Nc2ccc(Cl)nc2-c2ccc3c(c2)COB3O)c2oc(N3CCC(F)(F)CC3)c(C)c(=O)c2c1]

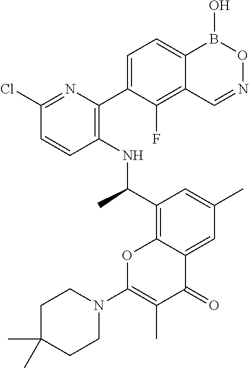

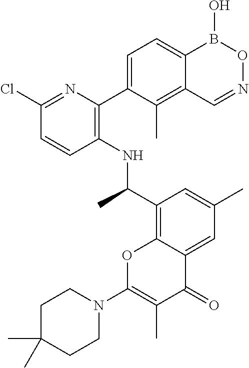

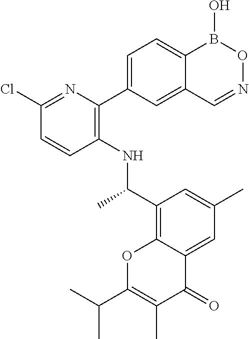 [SMILES: Cc1cc([C@H](C)Nc2ccc(Cl)nc2-c2ccc3c(c2)C=NOB3O)c2oc(C(C)C)c(C)c(=O)c2c1]

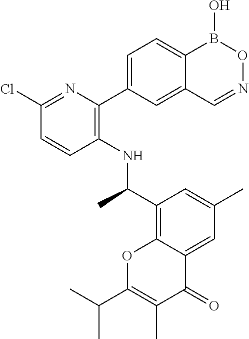 [SMILES: Cc1cc([C@@H](C)Nc2ccc(Cl)nc2-c2ccc3c(c2)C=NOB3O)c2oc(C(C)C)c(C)c(=O)c2c1]

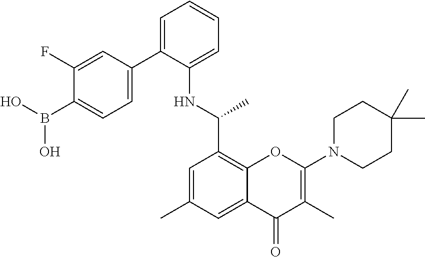 [SMILES: Cc1cc([C@@H](C)Nc2ccccc2-c2ccc(B(O)O)c(F)c2)c2oc(N3CCC(C)(C)CC3)c(C)c(=O)c2c1]

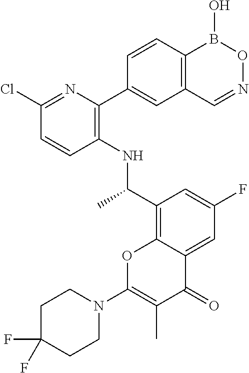 [SMILES: Cc1c(N2CCC(F)(F)CC2)oc2c([C@H](C)Nc3ccc(Cl)nc3-c3ccc4c(c3)C=NOB4O)cc(F)cc2c1=O]

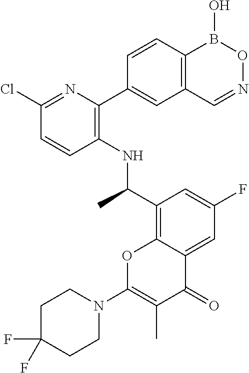 [SMILES: Cc1c(N2CCC(F)(F)CC2)oc2c([C@@H](C)Nc3ccc(Cl)nc3-c3ccc4c(c3)C=NOB4O)cc(F)cc2c1=O]

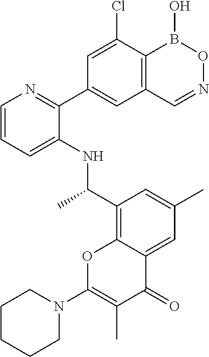 [SMILES: Cc1cc([C@H](C)Nc2cccnc2-c2cc(Cl)c3c(c2)C=NOB3O)c2oc(N3CCCCC3)c(C)c(=O)c2c1]

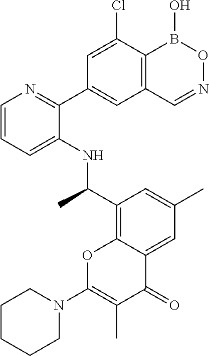 [SMILES: Cc1cc([C@@H](C)Nc2cccnc2-c2cc(Cl)c3c(c2)C=NOB3O)c2oc(N3CCCCC3)c(C)c(=O)c2c1]

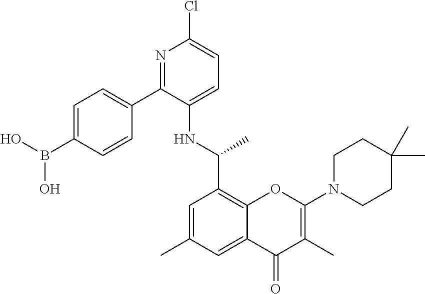 [SMILES: Cc1cc([C@@H](C)Nc2ccc(Cl)nc2-c2ccc(B(O)O)cc2)c2oc(N3CCC(C)(C)CC3)c(C)c(=O)c2c1]

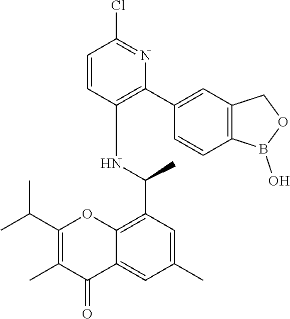 [SMILES: Cc1cc([C@H](C)Nc2ccc(Cl)nc2-c2ccc3c(c2)COB3O)c2oc(C(C)C)c(C)c(=O)c2c1]

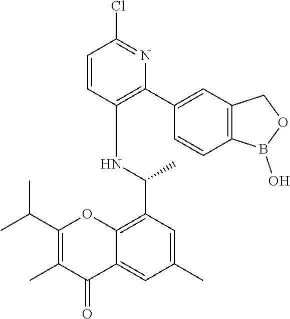 [SMILES: Cc1cc([C@@H](C)Nc2ccc(Cl)nc2-c2ccc3c(c2)COB3O)c2oc(C(C)C)c(C)c(=O)c2c1]

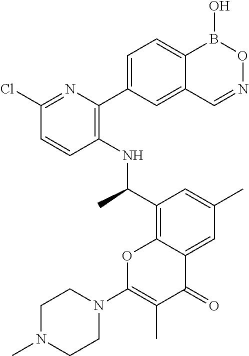 [SMILES: Cc1cc([C@@H](C)Nc2ccc(Cl)nc2-c2ccc3c(c2)C=NOB3O)c2oc(N3CCN(C)CC3)c(C)c(=O)c2c1]

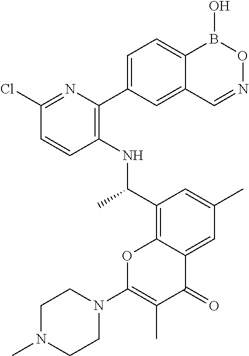 [SMILES: Cc1cc([C@H](C)Nc2ccc(Cl)nc2-c2ccc3c(c2)C=NOB3O)c2oc(N3CCN(C)CC3)c(C)c(=O)c2c1]

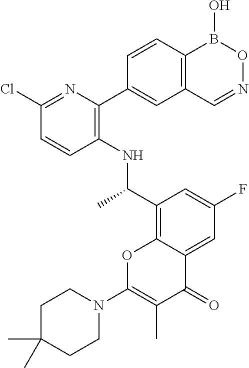 [SMILES: Cc1c(N2CCC(C)(C)CC2)oc2c([C@H](C)Nc3ccc(Cl)nc3-c3ccc4c(c3)C=NOB4O)cc(F)cc2c1=O]

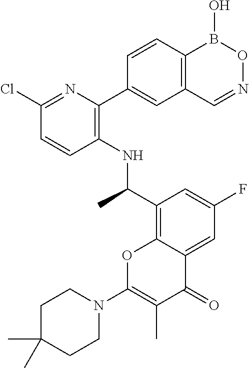 [SMILES: Cc1c(N2CCC(C)(C)CC2)oc2c([C@@H](C)Nc3ccc(Cl)nc3-c3ccc4c(c3)C=NOB4O)cc(F)cc2c1=O]

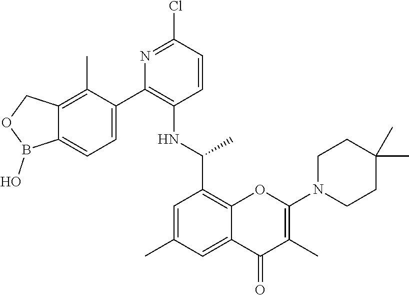 [SMILES: Cc1cc([C@@H](C)Nc2ccc(Cl)nc2-c2ccc3c(c2C)COB3O)c2oc(N3CCC(C)(C)CC3)c(C)c(=O)c2c1]

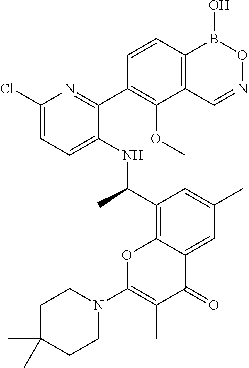

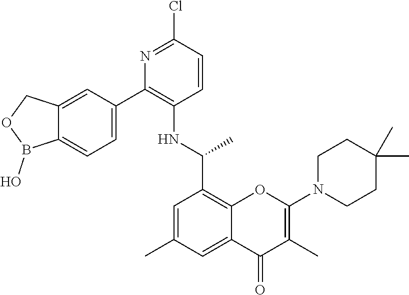 [SMILES: Cc1cc([C@@H](C)Nc2ccc(Cl)nc2-c2ccc3c(c2)COB3O)c2oc(N3CCC(C)(C)CC3)c(C)c(=O)c2c1]

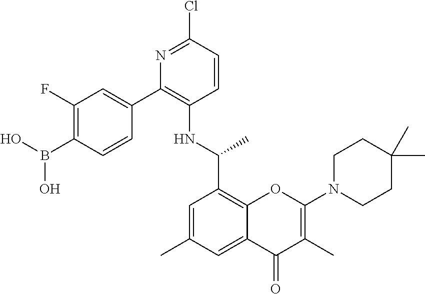 [SMILES: Cc1cc([C@@H](C)Nc2ccc(Cl)nc2-c2ccc(B(O)O)c(F)c2)c2oc(N3CCC(C)(C)CC3)c(C)c(=O)c2c1]

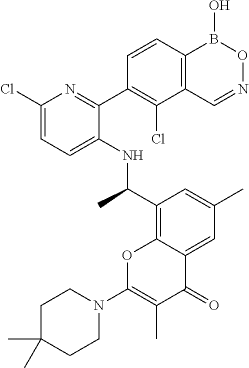

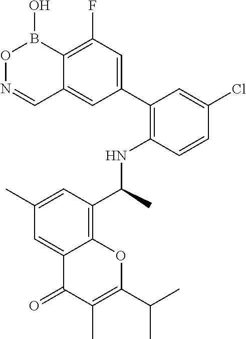 [SMILES: Cc1cc([C@H](C)Nc2ccc(Cl)cc2-c2cc(F)c3c(c2)C=NOB3O)c2oc(C(C)C)c(C)c(=O)c2c1]

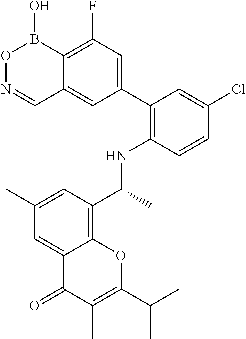 [SMILES: Cc1cc([C@@H](C)Nc2ccc(Cl)cc2-c2cc(F)c3c(c2)C=NOB3O)c2oc(C(C)C)c(C)c(=O)c2c1]

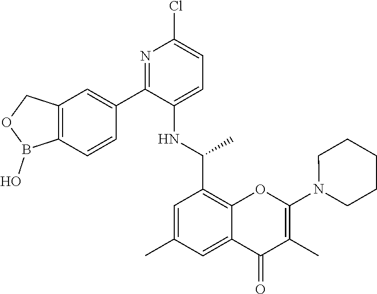 [SMILES: Cc1cc([C@@H](C)Nc2ccc(Cl)nc2-c2ccc3c(c2)COB3O)c2oc(N3CCCCC3)c(C)c(=O)c2c1]

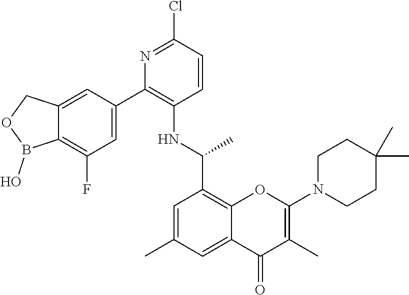 [SMILES: Cc1cc([C@@H](C)Nc2ccc(Cl)nc2-c2cc(F)c3c(c2)COB3O)c2oc(N3CCC(C)(C)CC3)c(C)c(=O)c2c1]

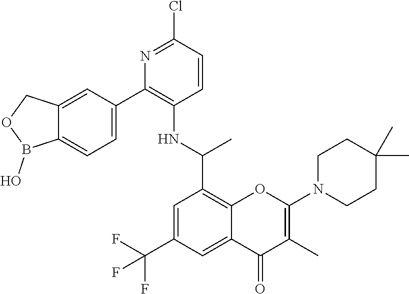 [SMILES: Cc1c(N2CCC(C)(C)CC2)oc2c(C(C)Nc3ccc(Cl)nc3-c3ccc4c(c3)COB4O)cc(C(F)(F)F)cc2c1=O]

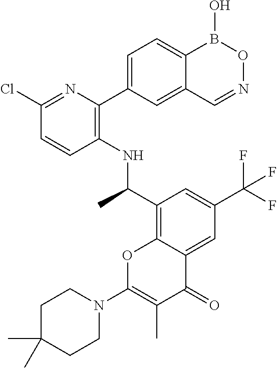 [SMILES: Cc1c(N2CCC(C)(C)CC2)oc2c([C@@H](C)Nc3ccc(Cl)nc3-c3ccc4c(c3)C=NOB4O)cc(C(F)(F)F)cc2c1=O]

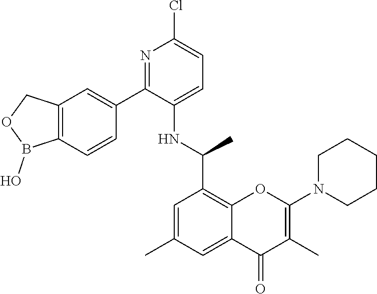 [SMILES: Cc1cc([C@H](C)Nc2ccc(Cl)nc2-c2ccc3c(c2)COB3O)c2oc(N3CCCCC3)c(C)c(=O)c2c1]

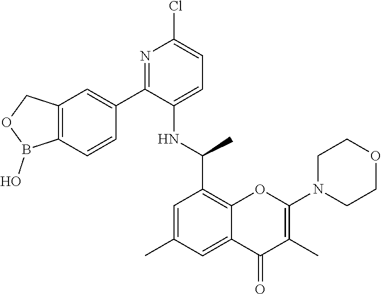 [SMILES: Cc1cc([C@H](C)Nc2ccc(Cl)nc2-c2ccc3c(c2)COB3O)c2oc(N3CCOCC3)c(C)c(=O)c2c1]

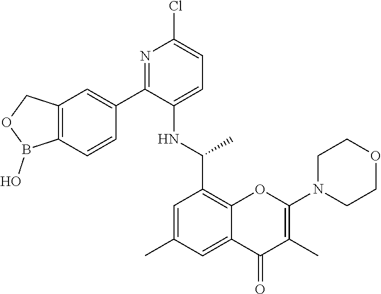 [SMILES: Cc1cc([C@@H](C)Nc2ccc(Cl)nc2-c2ccc3c(c2)COB3O)c2oc(N3CCOCC3)c(C)c(=O)c2c1]

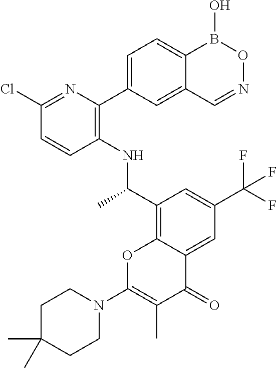 [SMILES: Cc1c(N2CCC(C)(C)CC2)oc2c([C@H](C)Nc3ccc(Cl)nc3-c3ccc4c(c3)C=NOB4O)cc(C(F)(F)F)cc2c1=O]

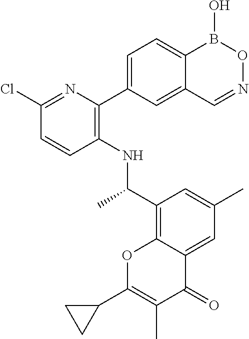 [SMILES: Cc1cc([C@H](C)Nc2ccc(Cl)nc2-c2ccc3c(c2)C=NOB3O)c2oc(C3CC3)c(C)c(=O)c2c1]

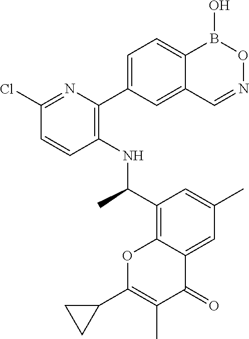 [SMILES: Cc1cc([C@@H](C)Nc2ccc(Cl)nc2-c2ccc3c(c2)C=NOB3O)c2oc(C3CC3)c(C)c(=O)c2c1]

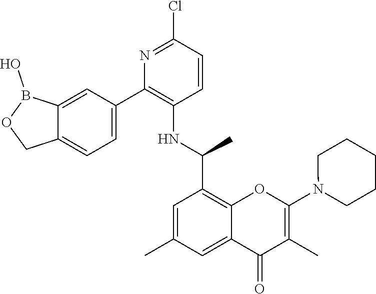 [SMILES: Cc1cc([C@H](C)Nc2ccc(Cl)nc2-c2ccc3c(c2)B(O)OC3)c2oc(N3CCCCC3)c(C)c(=O)c2c1]

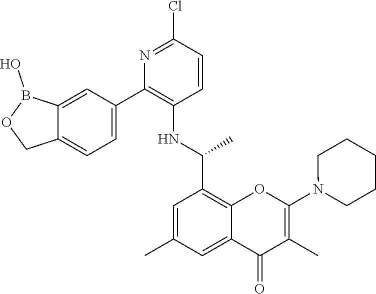 [SMILES: Cc1cc([C@@H](C)Nc2ccc(Cl)nc2-c2ccc3c(c2)B(O)OC3)c2oc(N3CCCCC3)c(C)c(=O)c2c1]

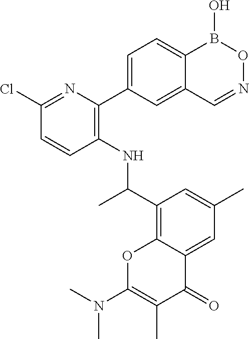 [SMILES: Cc1cc(C(C)Nc2ccc(Cl)nc2-c2ccc3c(c2)C=NOB3O)c2oc(N(C)C)c(C)c(=O)c2c1]

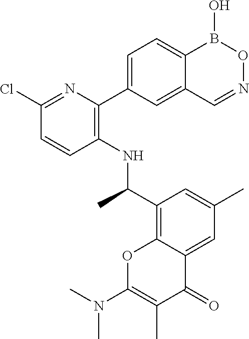 [SMILES: Cc1cc([C@@H](C)Nc2ccc(Cl)nc2-c2ccc3c(c2)C=NOB3O)c2oc(N(C)C)c(C)c(=O)c2c1]

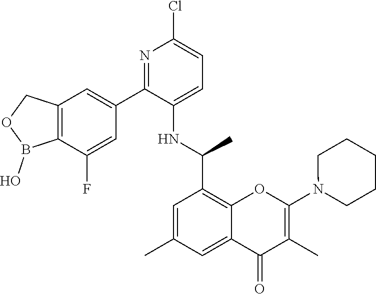 [SMILES: Cc1cc([C@H](C)Nc2ccc(Cl)nc2-c2cc(F)c3c(c2)COB3O)c2oc(N3CCCCC3)c(C)c(=O)c2c1]

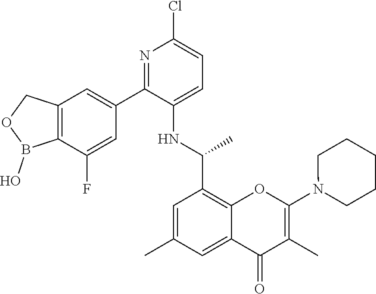 [SMILES: Cc1cc([C@@H](C)Nc2ccc(Cl)nc2-c2cc(F)c3c(c2)COB3O)c2oc(N3CCCCC3)c(C)c(=O)c2c1]

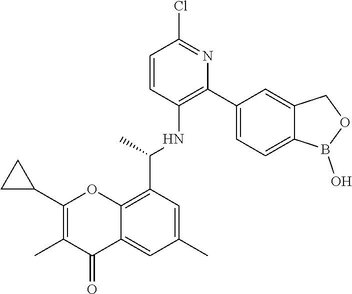 [SMILES: Cc1cc([C@H](C)Nc2ccc(Cl)nc2-c2ccc3c(c2)COB3O)c2oc(C3CC3)c(C)c(=O)c2c1]

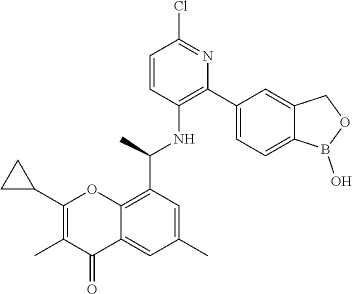 [SMILES: Cc1cc([C@@H](C)Nc2ccc(Cl)nc2-c2ccc3c(c2)COB3O)c2oc(C3CC3)c(C)c(=O)c2c1]

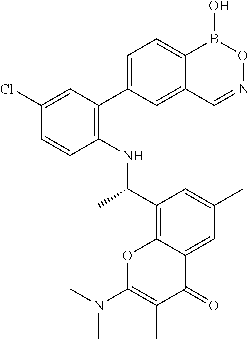 [SMILES: Cc1cc([C@H](C)Nc2ccc(Cl)cc2-c2ccc3c(c2)C=NOB3O)c2oc(N(C)C)c(C)c(=O)c2c1]

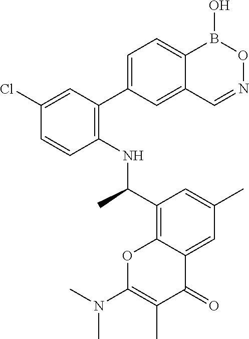 [SMILES: Cc1cc([C@@H](C)Nc2ccc(Cl)cc2-c2ccc3c(c2)C=NOB3O)c2oc(N(C)C)c(C)c(=O)c2c1]

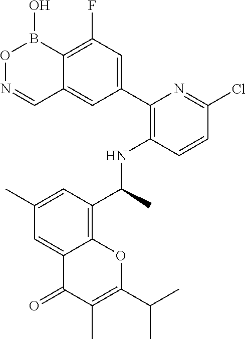 [SMILES: Cc1cc([C@H](C)Nc2ccc(Cl)nc2-c2cc(F)c3c(c2)C=NOB3O)c2oc(C(C)C)c(C)c(=O)c2c1]

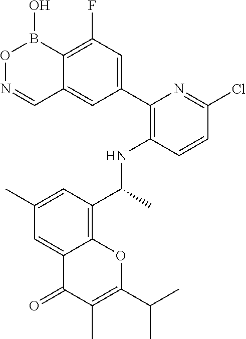 [SMILES: Cc1cc([C@@H](C)Nc2ccc(Cl)nc2-c2cc(F)c3c(c2)C=NOB3O)c2oc(C(C)C)c(C)c(=O)c2c1]

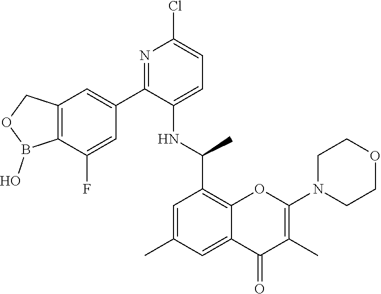 [SMILES: Cc1cc([C@H](C)Nc2ccc(Cl)nc2-c2cc(F)c3c(c2)COB3O)c2oc(N3CCOCC3)c(C)c(=O)c2c1]

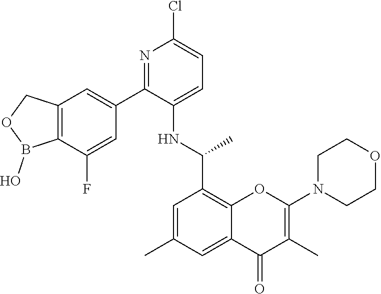 [SMILES: Cc1cc([C@@H](C)Nc2ccc(Cl)nc2-c2cc(F)c3c(c2)COB3O)c2oc(N3CCOCC3)c(C)c(=O)c2c1]

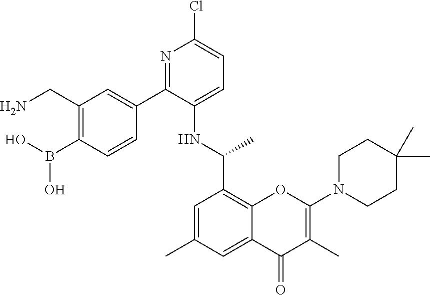 [SMILES: Cc1cc([C@@H](C)Nc2ccc(Cl)nc2-c2ccc(B(O)O)c(CN)c2)c2oc(N3CCC(C)(C)CC3)c(C)c(=O)c2c1]

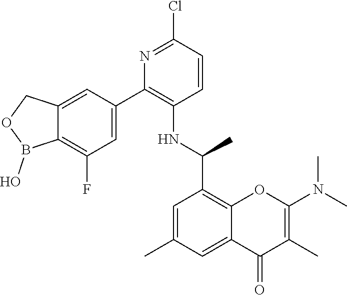 [SMILES: Cc1cc([C@H](C)Nc2ccc(Cl)nc2-c2cc(F)c3c(c2)COB3O)c2oc(N(C)C)c(C)c(=O)c2c1]

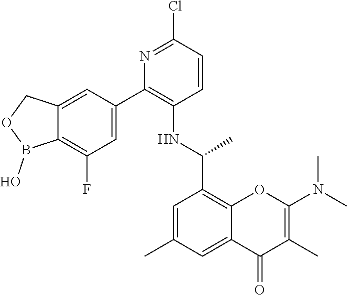 [SMILES: Cc1cc([C@@H](C)Nc2ccc(Cl)nc2-c2cc(F)c3c(c2)COB3O)c2oc(N(C)C)c(C)c(=O)c2c1]

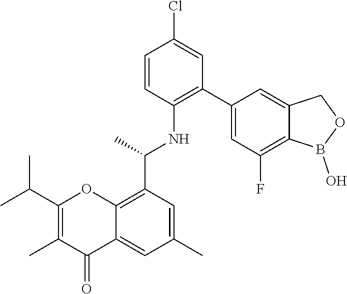 [SMILES: Cc1cc([C@H](C)Nc2ccc(Cl)cc2-c2cc(F)c3c(c2)COB3O)c2oc(C(C)C)c(C)c(=O)c2c1]

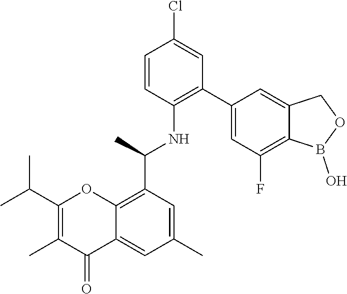 [SMILES: Cc1cc([C@@H](C)Nc2ccc(Cl)cc2-c2cc(F)c3c(c2)COB3O)c2oc(C(C)C)c(C)c(=O)c2c1]

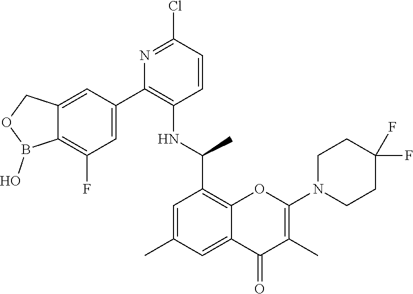 [SMILES: Cc1cc([C@H](C)Nc2ccc(Cl)nc2-c2cc(F)c3c(c2)COB3O)c2oc(N3CCC(F)(F)CC3)c(C)c(=O)c2c1]

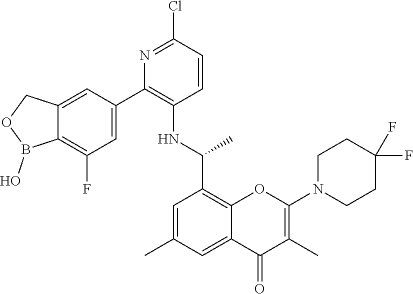 [SMILES: Cc1cc([C@@H](C)Nc2ccc(Cl)nc2-c2cc(F)c3c(c2)COB3O)c2oc(N3CCC(F)(F)CC3)c(C)c(=O)c2c1]

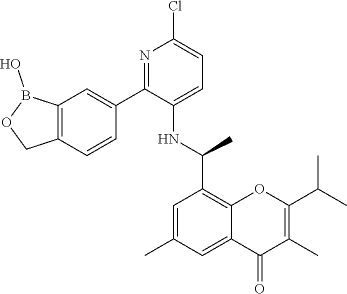 [SMILES: Cc1cc([C@H](C)Nc2ccc(Cl)nc2-c2ccc3c(c2)B(O)OC3)c2oc(C(C)C)c(C)c(=O)c2c1]

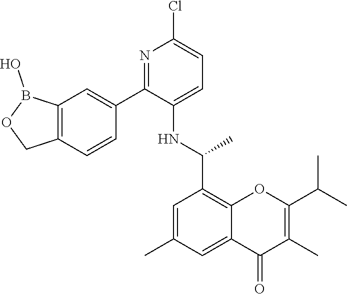 [SMILES: Cc1cc([C@@H](C)Nc2ccc(Cl)nc2-c2ccc3c(c2)B(O)OC3)c2oc(C(C)C)c(C)c(=O)c2c1]

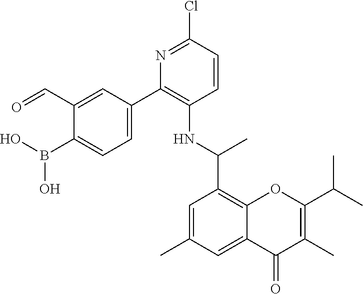 [SMILES: Cc1cc(C(C)Nc2ccc(Cl)nc2-c2ccc(B(O)O)c(C=O)c2)c2oc(C(C)C)c(C)c(=O)c2c1]

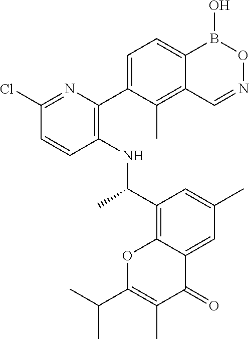 [SMILES: Cc1cc([C@H](C)Nc2ccc(Cl)nc2-c2ccc3c(c2C)C=NOB3O)c2oc(C(C)C)c(C)c(=O)c2c1]

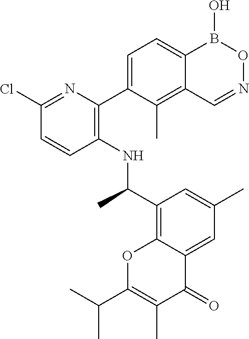 [SMILES: Cc1cc([C@@H](C)Nc2ccc(Cl)nc2-c2ccc3c(c2C)C=NOB3O)c2oc(C(C)C)c(C)c(=O)c2c1]

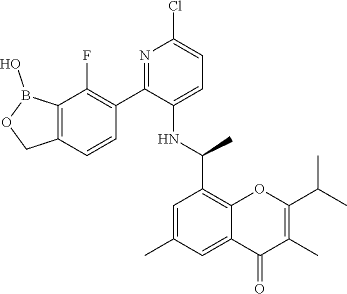 [SMILES: Cc1cc([C@H](C)Nc2ccc(Cl)nc2-c2ccc3c(c2F)B(O)OC3)c2oc(C(C)C)c(C)c(=O)c2c1]

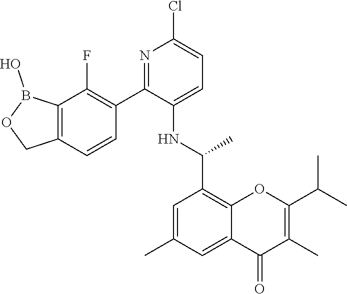 [SMILES: Cc1cc([C@@H](C)Nc2ccc(Cl)nc2-c2ccc3c(c2F)B(O)OC3)c2oc(C(C)C)c(C)c(=O)c2c1]

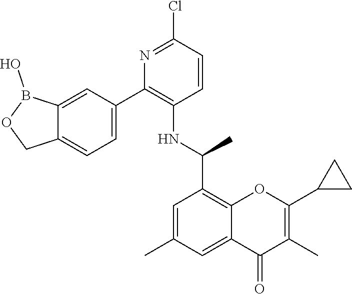 [SMILES: Cc1cc([C@H](C)Nc2ccc(Cl)nc2-c2ccc3c(c2)B(O)OC3)c2oc(C3CC3)c(C)c(=O)c2c1]

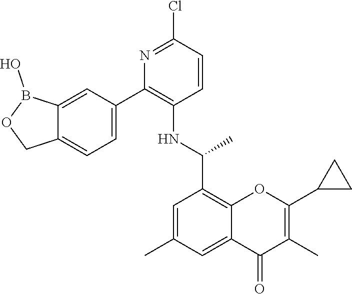 [SMILES: Cc1cc([C@@H](C)Nc2ccc(Cl)nc2-c2ccc3c(c2)B(O)OC3)c2oc(C3CC3)c(C)c(=O)c2c1]

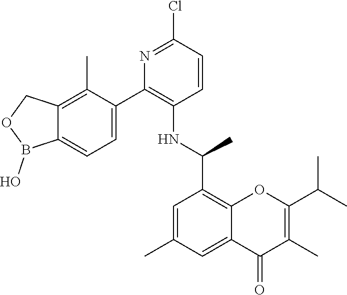 [SMILES: Cc1cc([C@H](C)Nc2ccc(Cl)nc2-c2ccc3c(c2C)COB3O)c2oc(C(C)C)c(C)c(=O)c2c1]

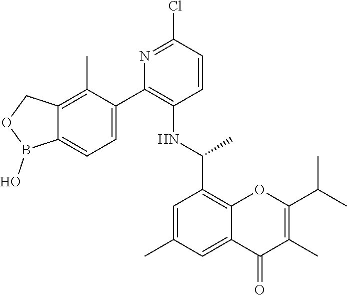 [SMILES: Cc1cc([C@@H](C)Nc2ccc(Cl)nc2-c2ccc3c(c2C)COB3O)c2oc(C(C)C)c(C)c(=O)c2c1]

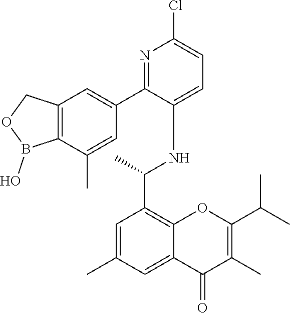 [SMILES: Cc1cc([C@H](C)Nc2ccc(Cl)nc2-c2cc(C)c3c(c2)COB3O)c2oc(C(C)C)c(C)c(=O)c2c1]

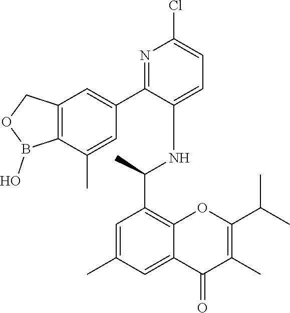 [SMILES: Cc1cc([C@@H](C)Nc2ccc(Cl)nc2-c2cc(C)c3c(c2)COB3O)c2oc(C(C)C)c(C)c(=O)c2c1]

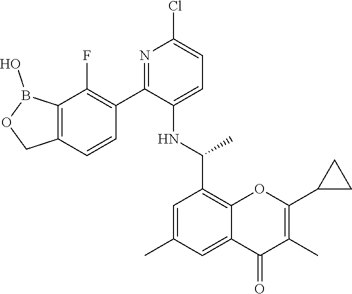 [SMILES: Cc1cc([C@@H](C)Nc2ccc(Cl)nc2-c2ccc3c(c2F)B(O)OC3)c2oc(C3CC3)c(C)c(=O)c2c1]

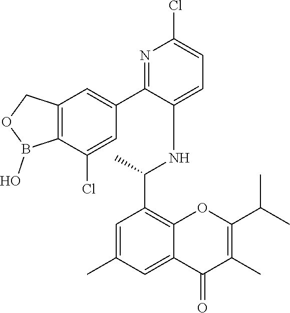 [SMILES: Cc1cc([C@H](C)Nc2ccc(Cl)nc2-c2cc(Cl)c3c(c2)COB3O)c2oc(C(C)C)c(C)c(=O)c2c1]

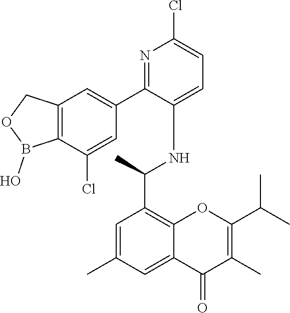 [SMILES: Cc1cc([C@@H](C)Nc2ccc(Cl)nc2-c2cc(Cl)c3c(c2)COB3O)c2oc(C(C)C)c(C)c(=O)c2c1]

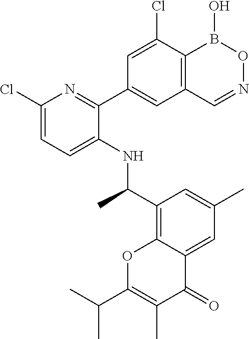 [SMILES: Cc1cc([C@@H](C)Nc2ccc(Cl)nc2-c2cc(Cl)c3c(c2)C=NOB3O)c2oc(C(C)C)c(C)c(=O)c2c1]

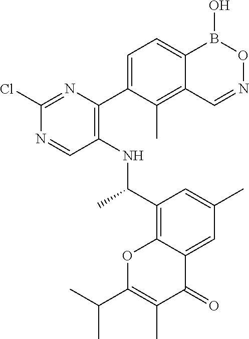 [SMILES: Cc1cc([C@H](C)Nc2cnc(Cl)nc2-c2ccc3c(c2C)C=NOB3O)c2oc(C(C)C)c(C)c(=O)c2c1]

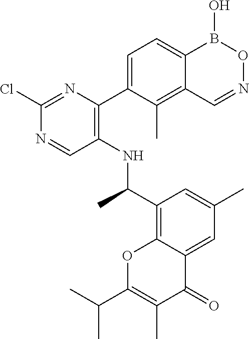 [SMILES: Cc1cc([C@@H](C)Nc2cnc(Cl)nc2-c2ccc3c(c2C)C=NOB3O)c2oc(C(C)C)c(C)c(=O)c2c1]

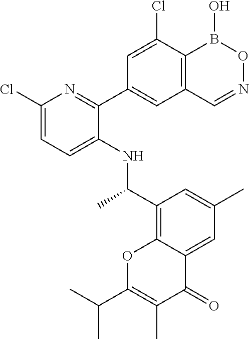 [SMILES: Cc1cc([C@H](C)Nc2ccc(Cl)nc2-c2cc(Cl)c3c(c2)C=NOB3O)c2oc(C(C)C)c(C)c(=O)c2c1]

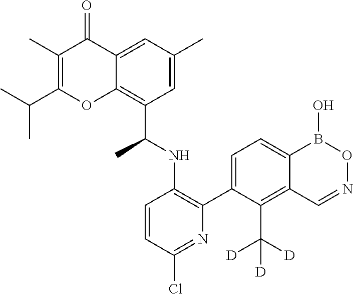 [SMILES: [2H]C([2H])([2H])c1c(-c2nc(Cl)ccc2N[C@@H](C)c2cc(C)cc3c(=O)c(C)c(C(C)C)oc23)ccc2c1C=NOB2O]

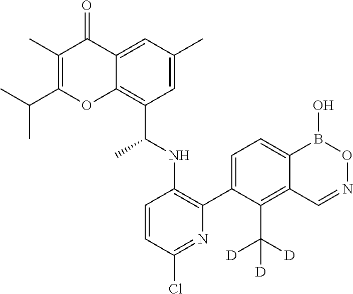 [SMILES: [2H]C([2H])([2H])c1c(-c2nc(Cl)ccc2N[C@H](C)c2cc(C)cc3c(=O)c(C)c(C(C)C)oc23)ccc2c1C=NOB2O]

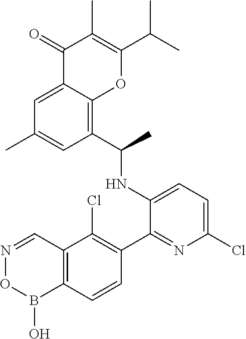 [SMILES: Cc1cc([C@@H](C)Nc2ccc(Cl)nc2-c2ccc3c(c2Cl)C=NOB3O)c2oc(C(C)C)c(C)c(=O)c2c1]

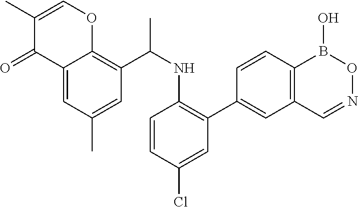 [SMILES: Cc1cc(C(C)Nc2ccc(Cl)cc2-c2ccc3c(c2)C=NOB3O)c2occ(C)c(=O)c2c1]

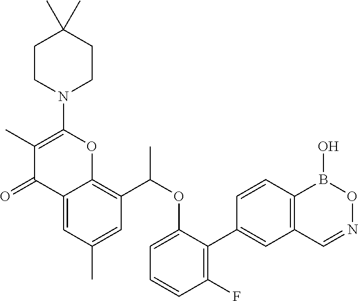 [SMILES: Cc1cc(C(C)Oc2cccc(F)c2-c2ccc3c(c2)C=NOB3O)c2oc(N3CCC(C)(C)CC3)c(C)c(=O)c2c1]

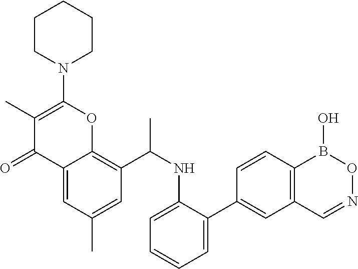 [SMILES: Cc1cc(C(C)Nc2ccccc2-c2ccc3c(c2)C=NOB3O)c2oc(N3CCCCC3)c(C)c(=O)c2c1]

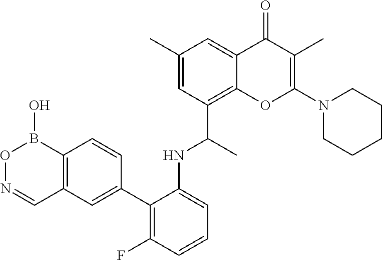 [SMILES: Cc1cc(C(C)Nc2cccc(F)c2-c2ccc3c(c2)C=NOB3O)c2oc(N3CCCCC3)c(C)c(=O)c2c1]

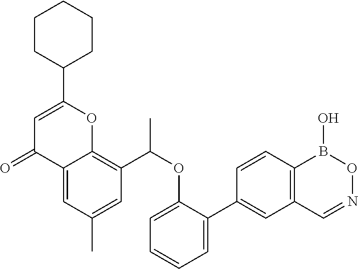 [SMILES: Cc1cc(C(C)Oc2ccccc2-c2ccc3c(c2)C=NOB3O)c2oc(C3CCCCC3)cc(=O)c2c1]

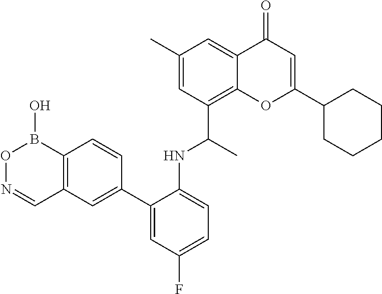 [SMILES: Cc1cc(C(C)Nc2ccc(F)cc2-c2ccc3c(c2)C=NOB3O)c2oc(C3CCCCC3)cc(=O)c2c1]

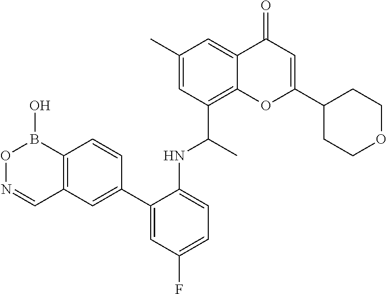 [SMILES: Cc1cc(C(C)Nc2ccc(F)cc2-c2ccc3c(c2)C=NOB3O)c2oc(C3CCOCC3)cc(=O)c2c1]

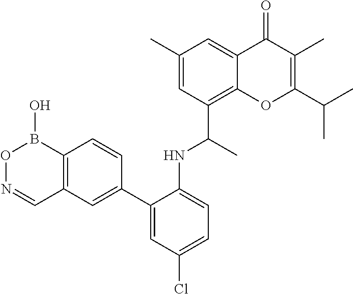 [SMILES: Cc1cc(C(C)Nc2ccc(Cl)cc2-c2ccc3c(c2)C=NOB3O)c2oc(C(C)C)c(C)c(=O)c2c1]

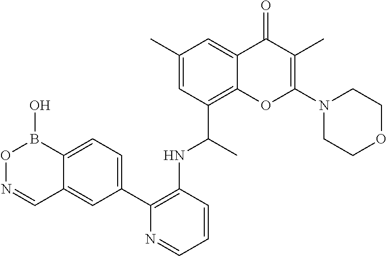 [SMILES: Cc1cc(C(C)Nc2cccnc2-c2ccc3c(c2)C=NOB3O)c2oc(N3CCOCC3)c(C)c(=O)c2c1]

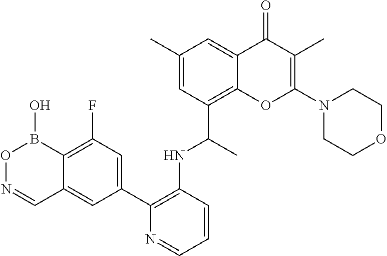 [SMILES: Cc1cc(C(C)Nc2cccnc2-c2cc(F)c3c(c2)C=NOB3O)c2oc(N3CCOCC3)c(C)c(=O)c2c1]

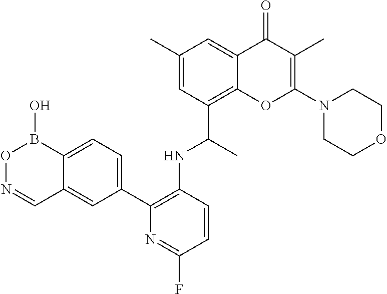 [SMILES: Cc1cc(C(C)Nc2ccc(F)nc2-c2ccc3c(c2)C=NOB3O)c2oc(N3CCOCC3)c(C)c(=O)c2c1]

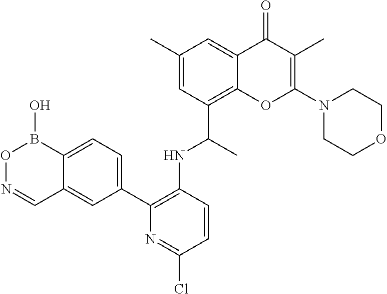 [SMILES: Cc1cc(C(C)Nc2ccc(Cl)nc2-c2ccc3c(c2)C=NOB3O)c2oc(N3CCOCC3)c(C)c(=O)c2c1]

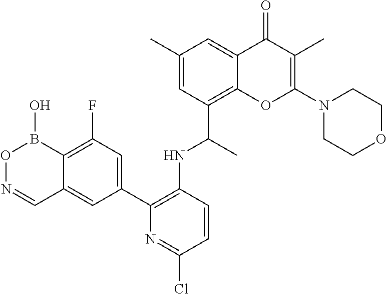 [SMILES: Cc1cc(C(C)Nc2ccc(Cl)nc2-c2cc(F)c3c(c2)C=NOB3O)c2oc(N3CCOCC3)c(C)c(=O)c2c1]

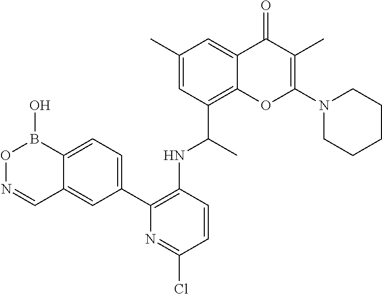 [SMILES: Cc1cc(C(C)Nc2ccc(Cl)nc2-c2ccc3c(c2)C=NOB3O)c2oc(N3CCCCC3)c(C)c(=O)c2c1]

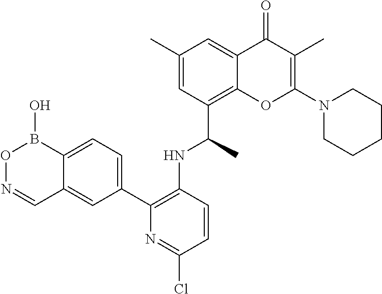 [SMILES: Cc1cc([C@@H](C)Nc2ccc(Cl)nc2-c2ccc3c(c2)C=NOB3O)c2oc(N3CCCCC3)c(C)c(=O)c2c1]

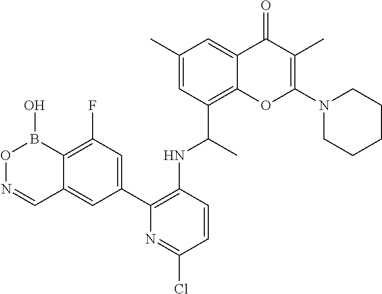 [SMILES: Cc1cc(C(C)Nc2ccc(Cl)nc2-c2cc(F)c3c(c2)C=NOB3O)c2oc(N3CCCCC3)c(C)c(=O)c2c1]

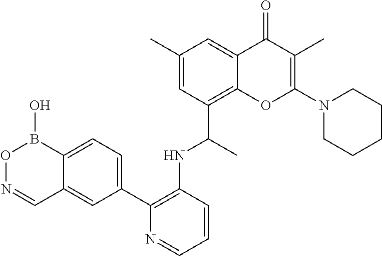 [SMILES: Cc1cc(C(C)Nc2cccnc2-c2ccc3c(c2)C=NOB3O)c2oc(N3CCCCC3)c(C)c(=O)c2c1]

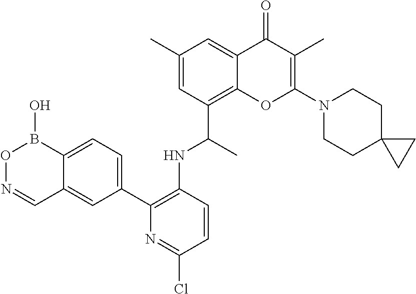 [SMILES: Cc1cc(C(C)Nc2ccc(Cl)nc2-c2ccc3c(c2)C=NOB3O)c2oc(N3CCC4(CC3)CC4)c(C)c(=O)c2c1]

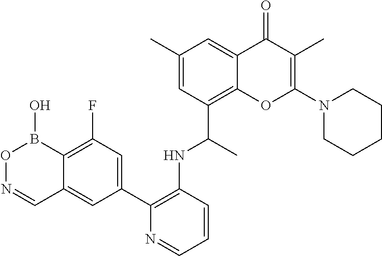 [SMILES: Cc1cc(C(C)Nc2cccnc2-c2cc(F)c3c(c2)C=NOB3O)c2oc(N3CCCCC3)c(C)c(=O)c2c1]

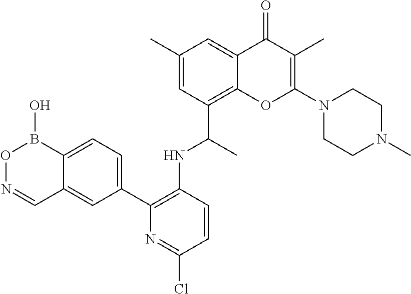 [SMILES: Cc1cc(C(C)Nc2ccc(Cl)nc2-c2ccc3c(c2)C=NOB3O)c2oc(N3CCN(C)CC3)c(C)c(=O)c2c1]

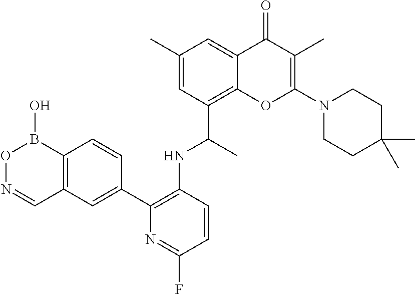 [SMILES: Cc1cc(C(C)Nc2ccc(F)nc2-c2ccc3c(c2)C=NOB3O)c2oc(N3CCC(C)(C)CC3)c(C)c(=O)c2c1]

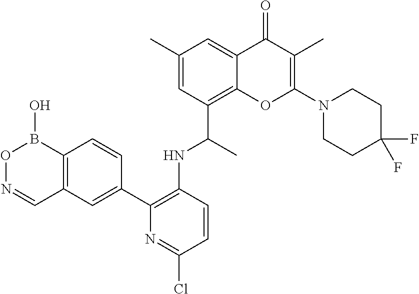 [SMILES: Cc1cc(C(C)Nc2ccc(Cl)nc2-c2ccc3c(c2)C=NOB3O)c2oc(N3CCC(F)(F)CC3)c(C)c(=O)c2c1]

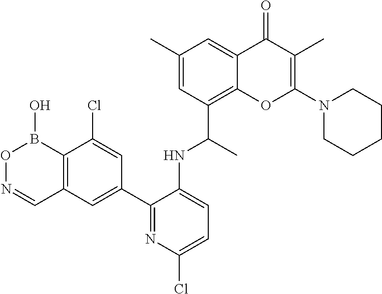 [SMILES: Cc1cc(C(C)Nc2ccc(Cl)nc2-c2cc(Cl)c3c(c2)C=NOB3O)c2oc(N3CCCCC3)c(C)c(=O)c2c1]

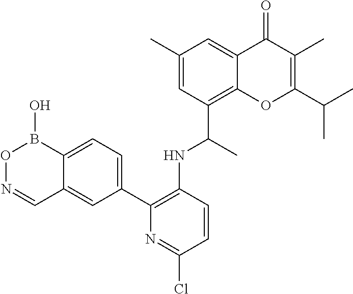 [SMILES: Cc1cc(C(C)Nc2ccc(Cl)nc2-c2ccc3c(c2)C=NOB3O)c2oc(C(C)C)c(C)c(=O)c2c1]

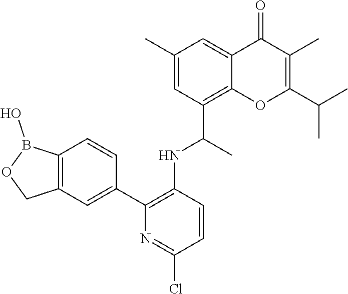 [SMILES: Cc1cc(C(C)Nc2ccc(Cl)nc2-c2ccc3c(c2)COB3O)c2oc(C(C)C)c(C)c(=O)c2c1]

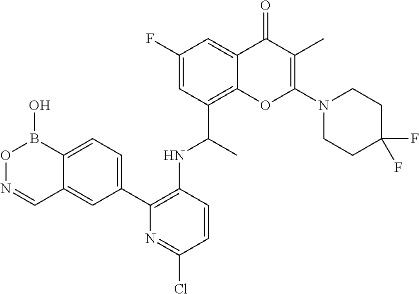 [SMILES: Cc1c(N2CCC(F)(F)CC2)oc2c(C(C)Nc3ccc(Cl)nc3-c3ccc4c(c3)C=NOB4O)cc(F)cc2c1=O]

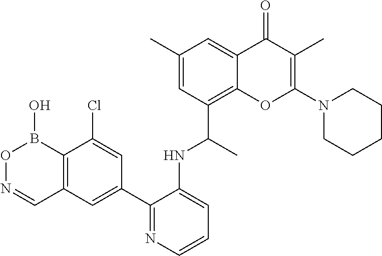 [SMILES: Cc1cc(C(C)Nc2cccnc2-c2cc(Cl)c3c(c2)C=NOB3O)c2oc(N3CCCCC3)c(C)c(=O)c2c1]

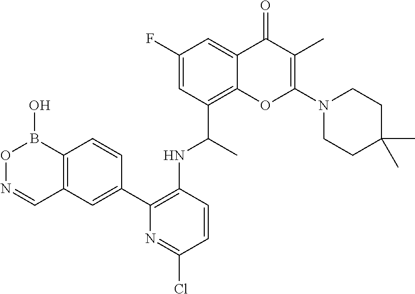 [SMILES: Cc1c(N2CCC(C)(C)CC2)oc2c(C(C)Nc3ccc(Cl)nc3-c3ccc4c(c3)C=NOB4O)cc(F)cc2c1=O]

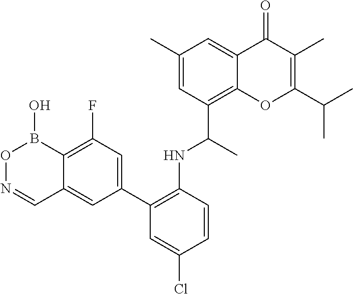 [SMILES: Cc1cc(C(C)Nc2ccc(Cl)cc2-c2cc(F)c3c(c2)C=NOB3O)c2oc(C(C)C)c(C)c(=O)c2c1]

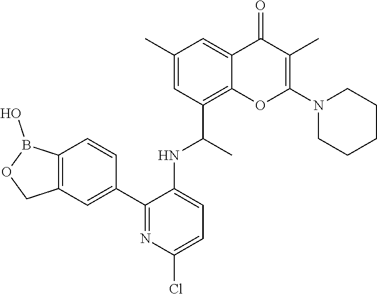 [SMILES: Cc1cc(C(C)Nc2ccc(Cl)nc2-c2ccc3c(c2)COB3O)c2oc(N3CCCCC3)c(C)c(=O)c2c1]

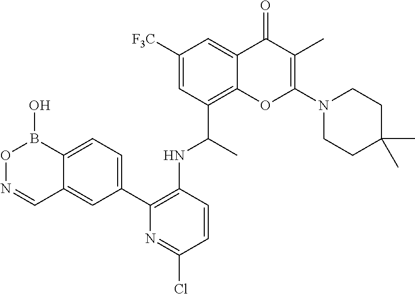 [SMILES: Cc1c(N2CCC(C)(C)CC2)oc2c(C(C)Nc3ccc(Cl)nc3-c3ccc4c(c3)C=NOB4O)cc(C(F)(F)F)cc2c1=O]

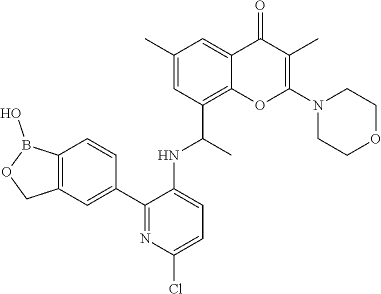 [SMILES: Cc1cc(C(C)Nc2ccc(Cl)nc2-c2ccc3c(c2)COB3O)c2oc(N3CCOCC3)c(C)c(=O)c2c1]

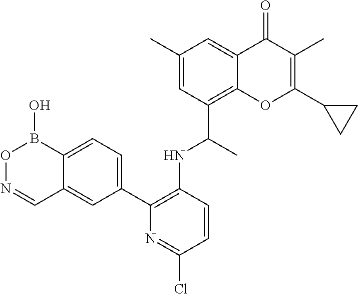 [SMILES: Cc1cc(C(C)Nc2ccc(Cl)nc2-c2ccc3c(c2)C=NOB3O)c2oc(C3CC3)c(C)c(=O)c2c1]

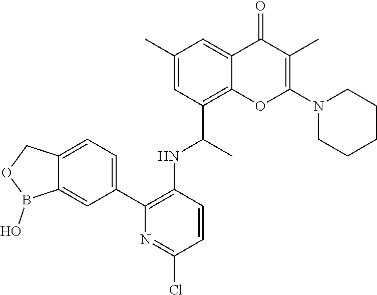 [SMILES: Cc1cc(C(C)Nc2ccc(Cl)nc2-c2ccc3c(c2)B(O)OC3)c2oc(N3CCCCC3)c(C)c(=O)c2c1]

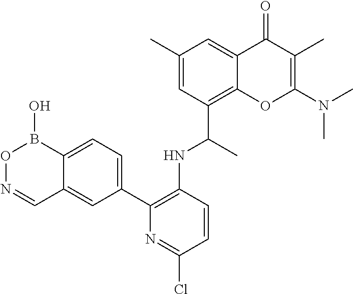 [SMILES: Cc1cc(C(C)Nc2ccc(Cl)nc2-c2ccc3c(c2)C=NOB3O)c2oc(N(C)C)c(C)c(=O)c2c1]

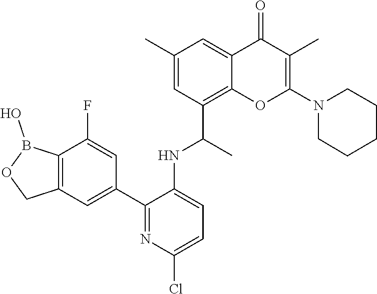 [SMILES: Cc1cc(C(C)Nc2ccc(Cl)nc2-c2cc(F)c3c(c2)COB3O)c2oc(N3CCCCC3)c(C)c(=O)c2c1]

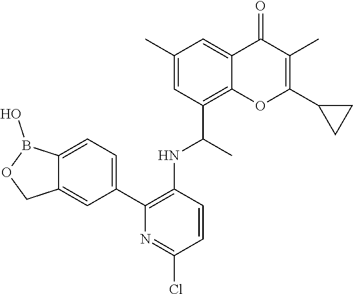 [SMILES: Cc1cc(C(C)Nc2ccc(Cl)nc2-c2ccc3c(c2)COB3O)c2oc(C3CC3)c(C)c(=O)c2c1]

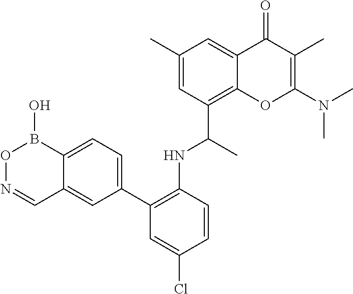 [SMILES: Cc1cc(C(C)Nc2ccc(Cl)cc2-c2ccc3c(c2)C=NOB3O)c2oc(N(C)C)c(C)c(=O)c2c1]

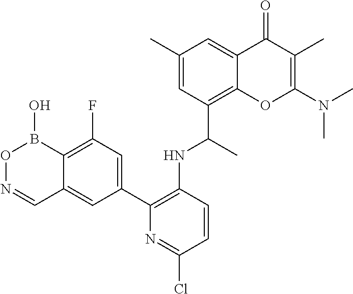 [SMILES: Cc1cc(C(C)Nc2ccc(Cl)nc2-c2cc(F)c3c(c2)C=NOB3O)c2oc(N(C)C)c(C)c(=O)c2c1]

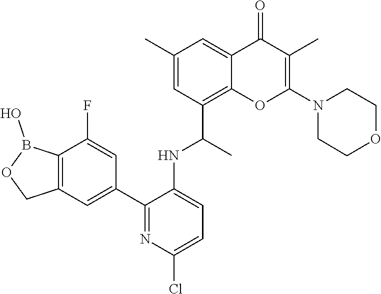 [SMILES: Cc1cc(C(C)Nc2ccc(Cl)nc2-c2cc(F)c3c(c2)COB3O)c2oc(N3CCOCC3)c(C)c(=O)c2c1]

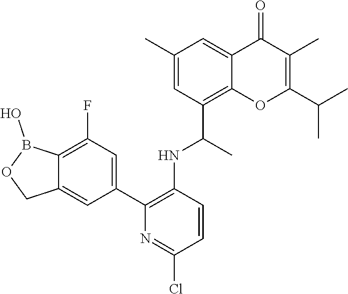 [SMILES: Cc1cc(C(C)Nc2ccc(Cl)nc2-c2cc(F)c3c(c2)COB3O)c2oc(C(C)C)c(C)c(=O)c2c1]

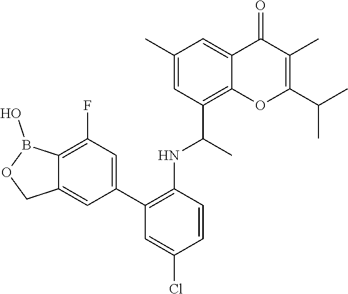 [SMILES: Cc1cc(C(C)Nc2ccc(Cl)cc2-c2cc(F)c3c(c2)COB3O)c2oc(C(C)C)c(C)c(=O)c2c1]

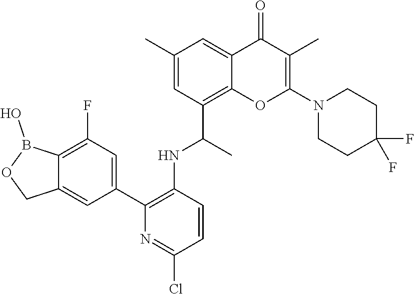 [SMILES: Cc1cc(C(C)Nc2ccc(Cl)nc2-c2cc(F)c3c(c2)COB3O)c2oc(N3CCC(F)(F)CC3)c(C)c(=O)c2c1]

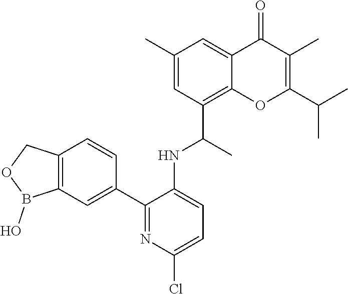 [SMILES: Cc1cc(C(C)Nc2ccc(Cl)nc2-c2ccc3c(c2)B(O)OC3)c2oc(C(C)C)c(C)c(=O)c2c1]

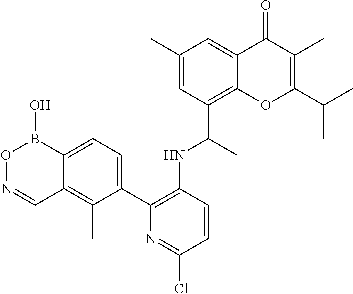 [SMILES: Cc1cc(C(C)Nc2ccc(Cl)nc2-c2ccc3c(c2C)C=NOB3O)c2oc(C(C)C)c(C)c(=O)c2c1]

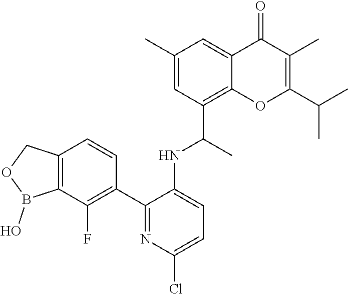 [SMILES: Cc1cc(C(C)Nc2ccc(Cl)nc2-c2ccc3c(c2F)B(O)OC3)c2oc(C(C)C)c(C)c(=O)c2c1]

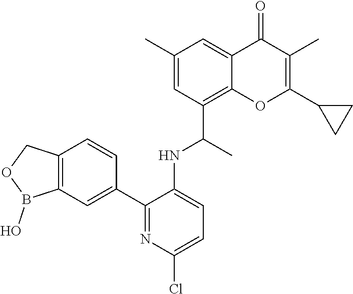 [SMILES: Cc1cc(C(C)Nc2ccc(Cl)nc2-c2ccc3c(c2)B(O)OC3)c2oc(C3CC3)c(C)c(=O)c2c1]

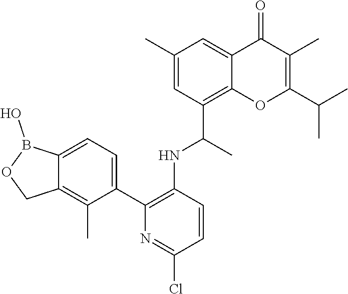 [SMILES: Cc1cc(C(C)Nc2ccc(Cl)nc2-c2ccc3c(c2C)COB3O)c2oc(C(C)C)c(C)c(=O)c2c1]

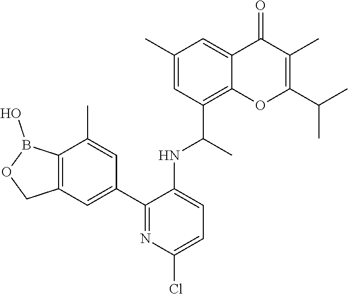 [SMILES: Cc1cc(C(C)Nc2ccc(Cl)nc2-c2cc(C)c3c(c2)COB3O)c2oc(C(C)C)c(C)c(=O)c2c1]

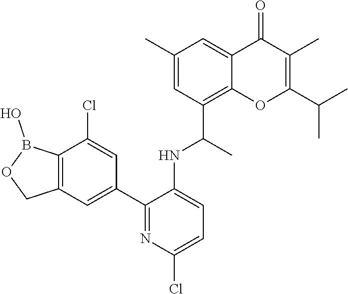 [SMILES: Cc1cc(C(C)Nc2ccc(Cl)nc2-c2cc(Cl)c3c(c2)COB3O)c2oc(C(C)C)c(C)c(=O)c2c1]

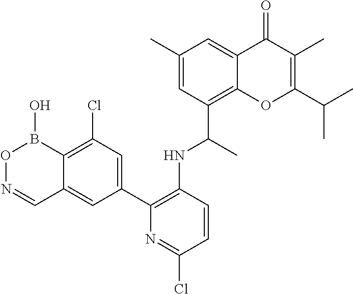 [SMILES: Cc1cc(C(C)Nc2ccc(Cl)nc2-c2cc(Cl)c3c(c2)C=NOB3O)c2oc(C(C)C)c(C)c(=O)c2c1]

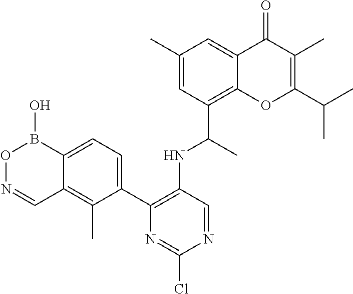 [SMILES: Cc1cc(C(C)Nc2cnc(Cl)nc2-c2ccc3c(c2C)C=NOB3O)c2oc(C(C)C)c(C)c(=O)c2c1]

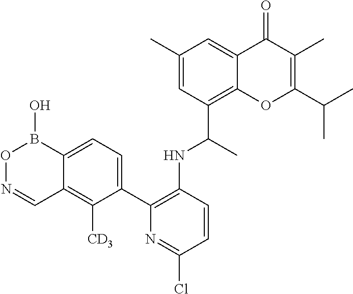 [SMILES: [2H]C([2H])([2H])c1c(-c2nc(Cl)ccc2NC(C)c2cc(C)cc3c(=O)c(C)c(C(C)C)oc23)ccc2c1C=NOB2O]

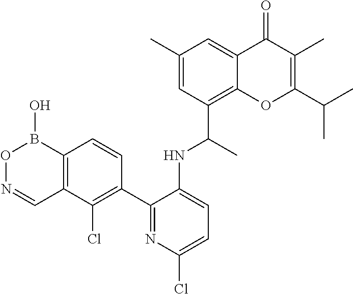 [SMILES: Cc1cc(C(C)Nc2ccc(Cl)nc2-c2ccc3c(c2Cl)C=NOB3O)c2oc(C(C)C)c(C)c(=O)c2c1]

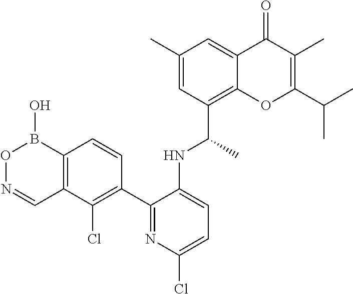 [SMILES: Cc1cc([C@H](C)Nc2ccc(Cl)nc2-c2ccc3c(c2Cl)C=NOB3O)c2oc(C(C)C)c(C)c(=O)c2c1]

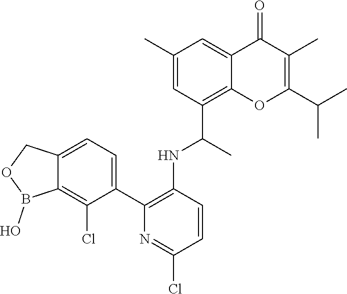 [SMILES: Cc1cc(C(C)Nc2ccc(Cl)nc2-c2ccc3c(c2Cl)B(O)OC3)c2oc(C(C)C)c(C)c(=O)c2c1]

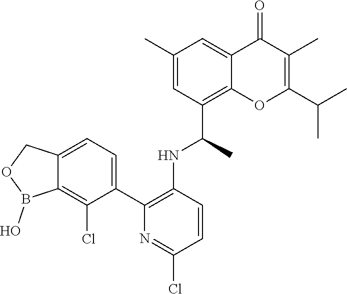 [SMILES: Cc1cc([C@@H](C)Nc2ccc(Cl)nc2-c2ccc3c(c2Cl)B(O)OC3)c2oc(C(C)C)c(C)c(=O)c2c1]

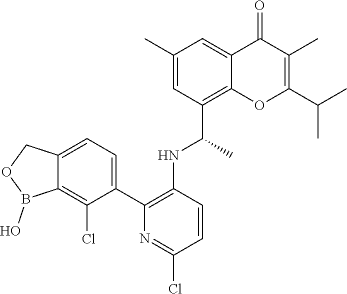 [SMILES: Cc1cc([C@H](C)Nc2ccc(Cl)nc2-c2ccc3c(c2Cl)B(O)OC3)c2oc(C(C)C)c(C)c(=O)c2c1]

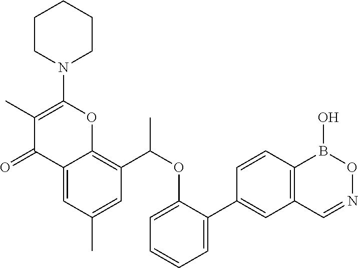 [SMILES: Cc1cc(C(C)Oc2ccccc2-c2ccc3c(c2)C=NOB3O)c2oc(N3CCCCC3)c(C)c(=O)c2c1]

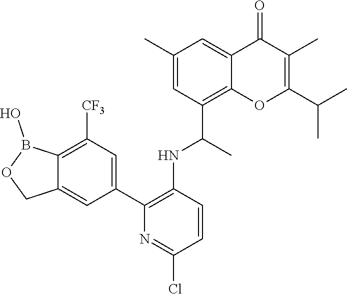 [SMILES: Cc1cc(C(C)Nc2ccc(Cl)nc2-c2cc3c(c(C(F)(F)F)c2)B(O)OC3)c2oc(C(C)C)c(C)c(=O)c2c1]

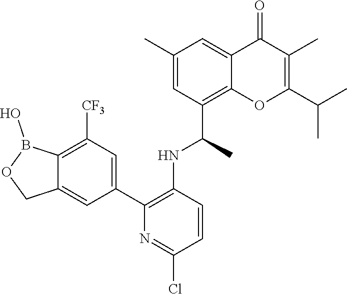 [SMILES: Cc1cc([C@@H](C)Nc2ccc(Cl)nc2-c2cc3c(c(C(F)(F)F)c2)B(O)OC3)c2oc(C(C)C)c(C)c(=O)c2c1]

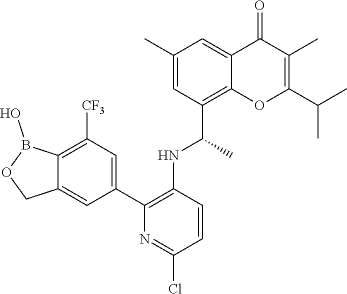 [SMILES: Cc1cc([C@H](C)Nc2ccc(Cl)nc2-c2cc3c(c(C(F)(F)F)c2)B(O)OC3)c2oc(C(C)C)c(C)c(=O)c2c1]

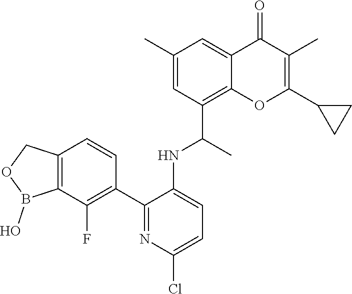 [SMILES: Cc1cc(C(C)Nc2ccc(Cl)nc2-c2ccc3c(c2F)B(O)OC3)c2oc(C3CC3)c(C)c(=O)c2c1]

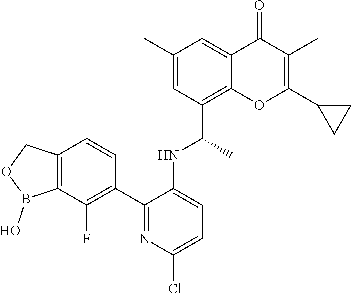 [SMILES: Cc1cc([C@H](C)Nc2ccc(Cl)nc2-c2ccc3c(c2F)B(O)OC3)c2oc(C3CC3)c(C)c(=O)c2c1]

One aspect of the present disclosure includes a compound of the present disclosure, or a tautomer, enantiomer, diastereomer, mixture, salt, hydrate, solvate, or deuterated form thereof, wherein the Y^{T} group, as well as the more specific recited groups identified in a comparable position to Y^{T}, is selected from Table 2.

TABLE 2
Each of which is unsubstitued or substituted:

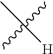 [SMILES: *[H]]

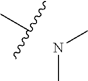

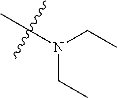 [SMILES: *N(CC)CC]

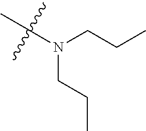 [SMILES: *N(CCC)CCC]

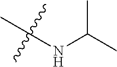 [SMILES: *NC(C)C]

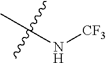 [SMILES: *NC(F)(F)F]

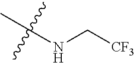 [SMILES: *NCC(F)(F)F]

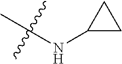 [SMILES: *NC1CC1]

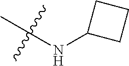 [SMILES: *NC1CCC1]

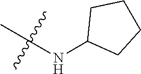 [SMILES: *NC1CCCC1]

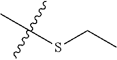 [SMILES: *SCC]

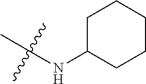 [SMILES: *NC1CCCCC1]

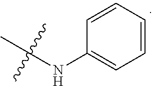 [SMILES: *Nc1ccccc1]

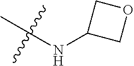 [SMILES: *NC1COC1]

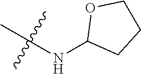 [SMILES: *NC1CCCO1]

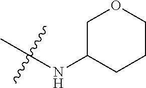 [SMILES: *NC1CCCOC1]

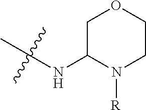

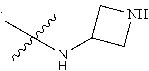 [SMILES: *NC1CNC1]

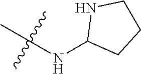 [SMILES: *NC1CCCN1]

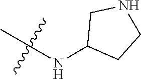 [SMILES: *NC1CCNC1]

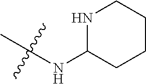 [SMILES: *NC1CCCCN1]

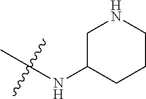 [SMILES: *NC1CCCNC1]

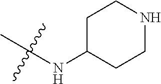 [SMILES: *NC1CCNCC1]

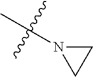 [SMILES: *N1CC1]

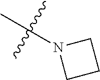 [SMILES: *N1CCC1]

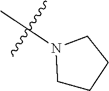 [SMILES: *N1CCCC1]

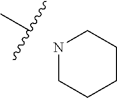

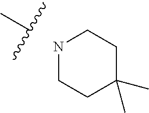

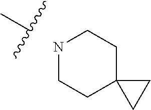

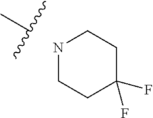

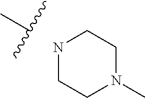

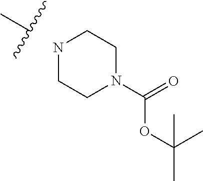

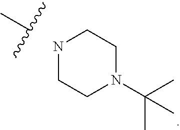

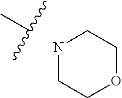

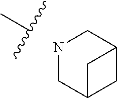

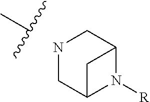

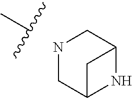

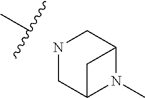

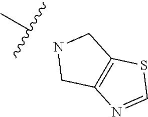

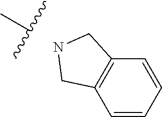

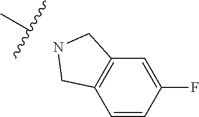

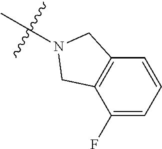 [SMILES: *N1Cc2cccc(F)c2C1]

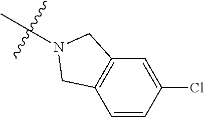 [SMILES: *N1Cc2ccc(Cl)cc2C1]

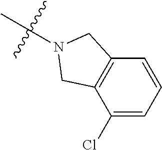 [SMILES: *N1Cc2cccc(Cl)c2C1]

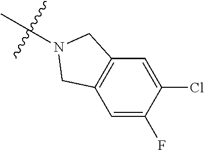 [SMILES: *N1Cc2cc(F)c(Cl)cc2C1]

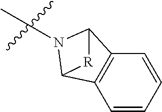

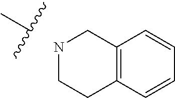

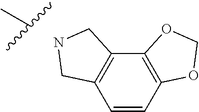

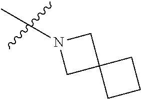 [SMILES: *N1CC2(CCC2)C1]

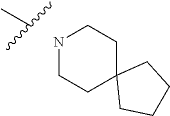

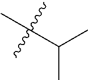 [SMILES: *C(C)C]

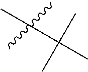 [SMILES: *C(C)(C)C]

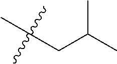 [SMILES: *CC(C)C]

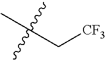 [SMILES: *CC(F)(F)F]

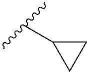 [SMILES: *C1CC1]

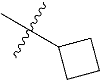 [SMILES: *C1CCC1]

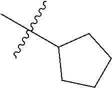 [SMILES: *C1CCCC1]

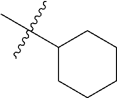 [SMILES: *C1CCCCC1]

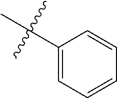 [SMILES: *c1ccccc1]

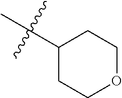 [SMILES: *C1CCOCC1]

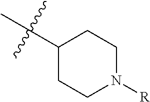

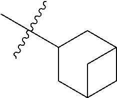 [SMILES: *C1CC2CC(C1)C2]

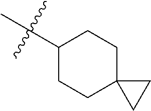 [SMILES: *C1CCC2(CC1)CC2]

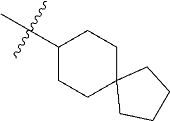 [SMILES: *C1CCC2(CCCC2)CC1]

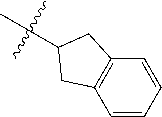 [SMILES: *C1Cc2ccccc2C1]

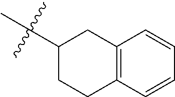 [SMILES: *C1CCc2ccccc2C1]

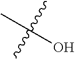 [SMILES: *O]

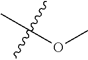 [SMILES: *OC]

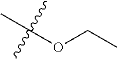 [SMILES: *OCC]

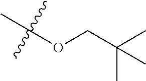 [SMILES: *OCC(C)(C)C]

and

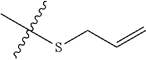 [SMILES: *SCC=C]

One aspect of the present disclosure includes a compound of the present disclosure, or a tautomer, enantiomer, diastereomer, mixture, salt, hydrate, solvate, or deuterated form thereof, wherein the linker group, as well as the more specific recited groups identified in a comparable position to L¹-L², is selected from Table 3.

TABLE 3

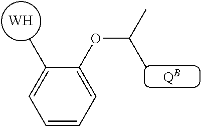

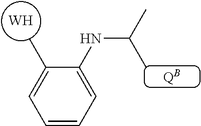

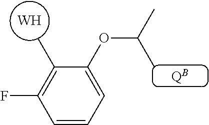

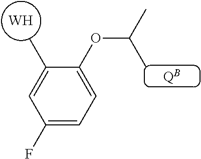

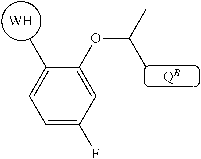

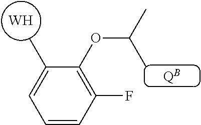

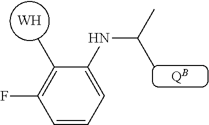

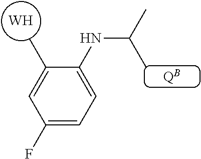

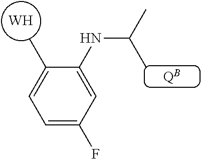

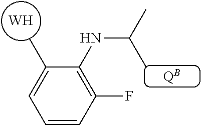

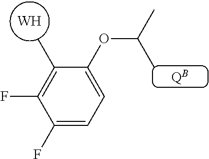

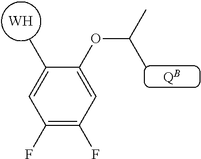

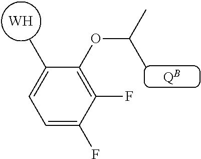

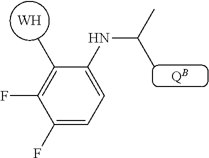

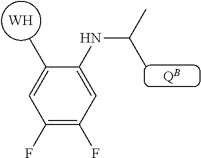

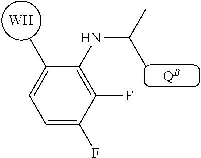

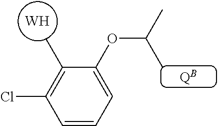

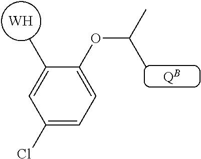

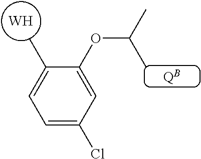

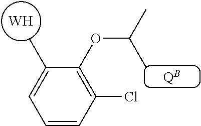

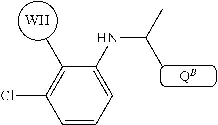

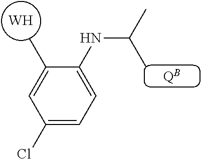

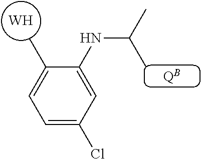

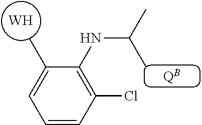

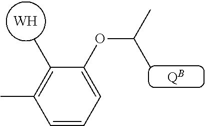

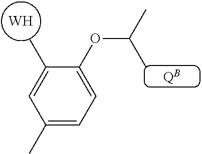

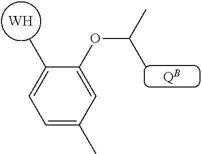

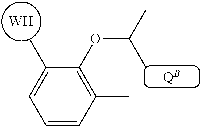

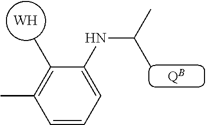

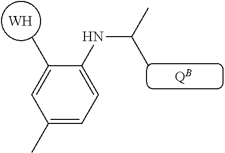

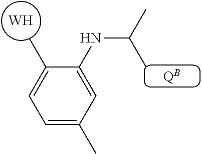

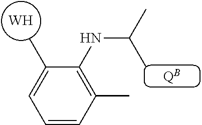

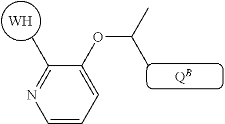

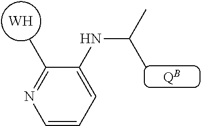

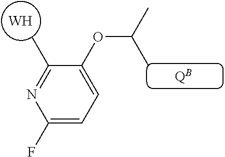

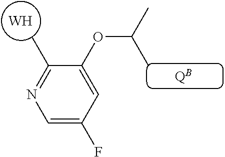

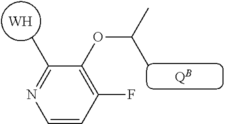

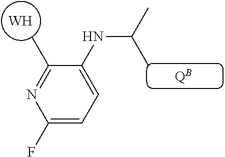

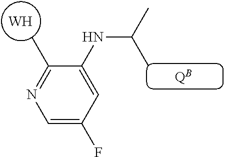

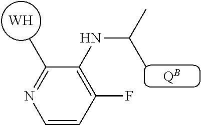

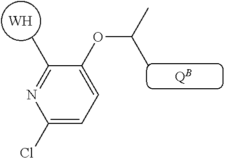

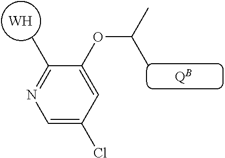

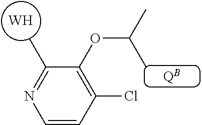

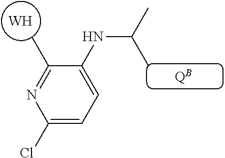

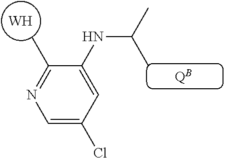

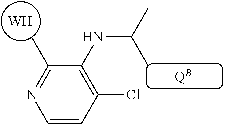

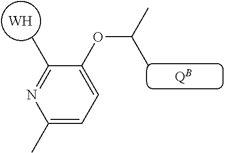

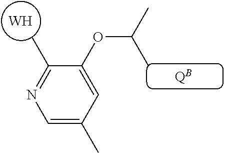

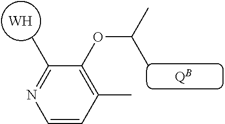

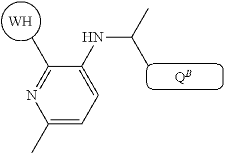

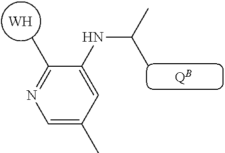

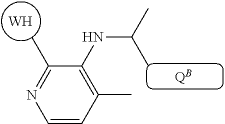

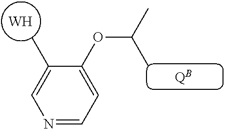

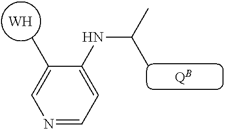

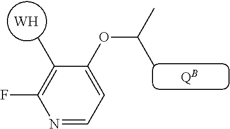

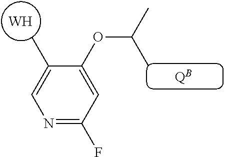

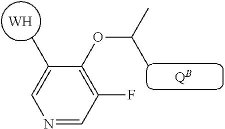

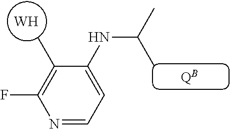

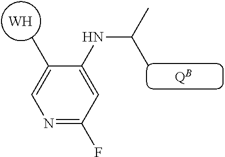

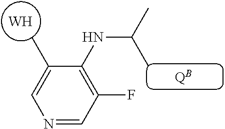

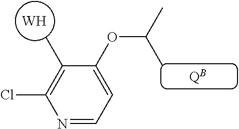

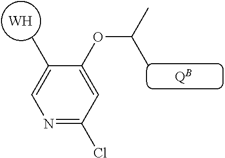

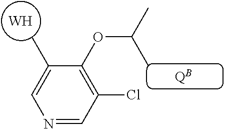

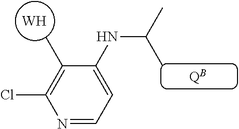

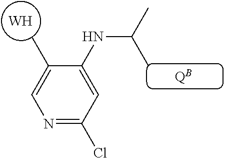

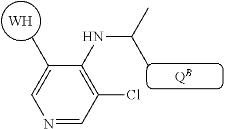

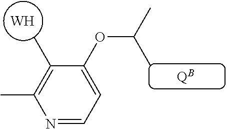

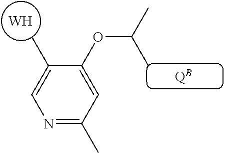

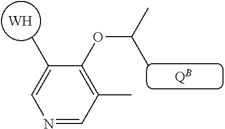

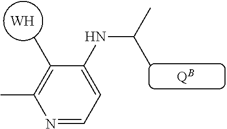

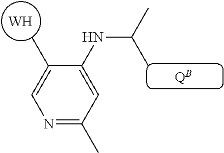

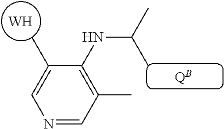

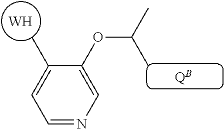

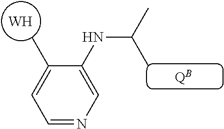

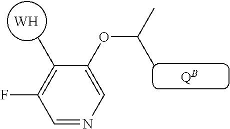

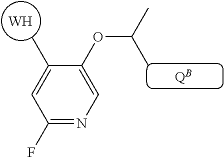

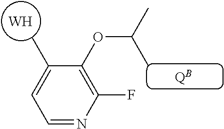

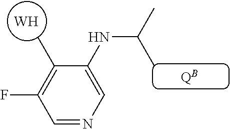

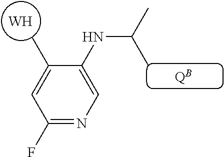

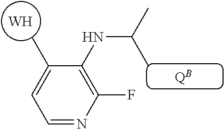

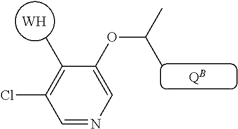

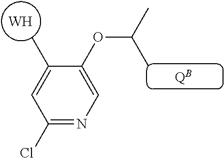

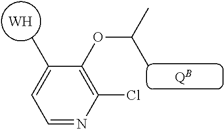

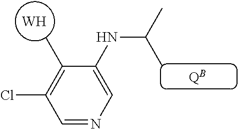

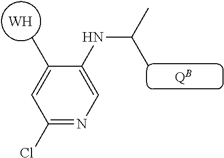

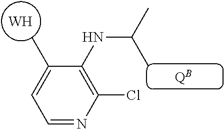

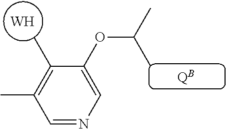

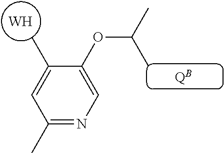

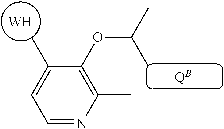

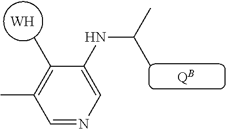

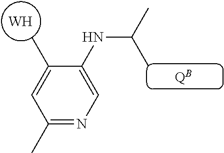

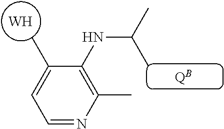

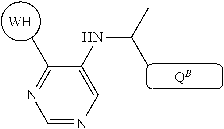

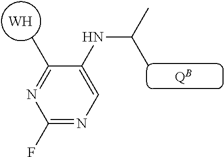

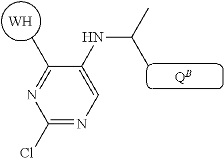

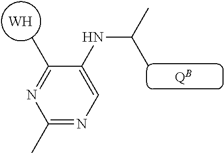

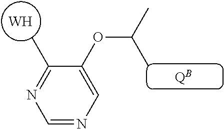

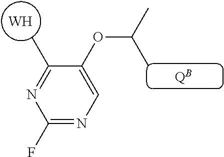

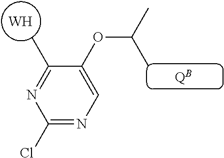

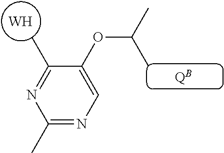

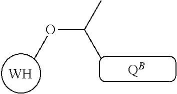

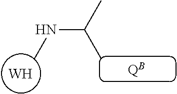

One aspect of the present disclosure includes a compound of the present disclosure, or a tautomer, enantiomer, diastereomer, mixture, salt, hydrate, solvate, or deuterated form thereof, wherein the Q^{B} group, as well as the more specific recited groups identified in a comparable position to Q^{B}, is selected from Table 4.

TABLE 4

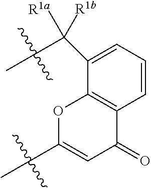

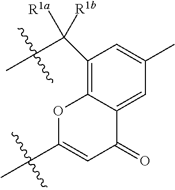

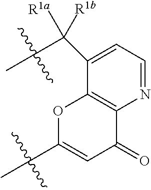

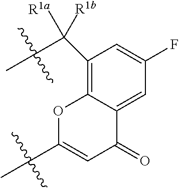

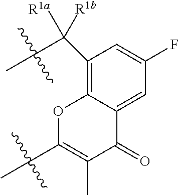

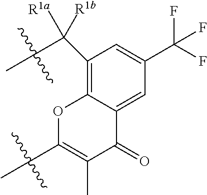

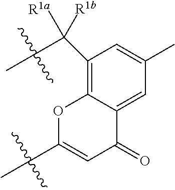

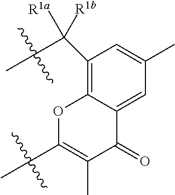

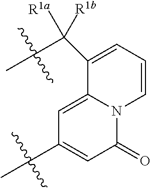

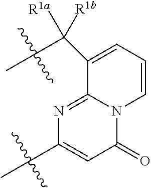

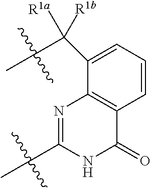

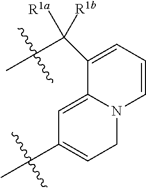

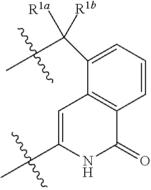

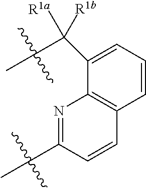

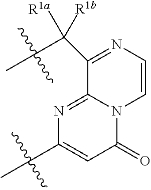

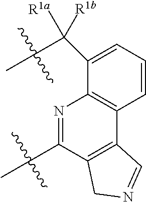

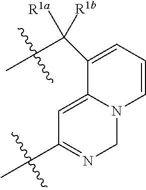

and

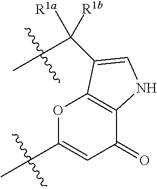

One or more aspects and embodiments may be incorporated in a different embodiment although not specifically described. That is, all aspects and embodiments may be combined in any way or combination.

### BRIEF DESCRIPTION OF THE DRAWINGS

FIG. 1 provides tabulated biological data for compounds of the present disclosure.

### DETAILED DESCRIPTION

### Definitions

When referring to the compounds disclosed herein, the following terms have the following meanings unless indicated otherwise. The following definitions are meant to clarify, but not limit, the terms defined. If a particular term used herein is not specifically defined, such term should not be considered indefinite. Rather, terms are used within their accepted meanings.

As used herein, “alkyl” refers to monovalent saturated aliphatic hydrocarbon groups having from 1 to 20 carbon atoms, preferably 1-8 carbon atoms, more preferably 1-6 carbon atoms. The hydrocarbon chain may be either straight-chained or branched. Illustrative alkyl groups include methyl, ethyl, n-propyl, iso-propyl, n-butyl, iso-butyl, and tert-butyl. Similarly, an “alkenyl” group refers to an alkyl group having one or more double bonds present in the chain, and an “alkynyl” group refers to an alkyl group having one or more triple bonds present in the chain.

As used herein “halogen” or “halo” refers to a halogen. In some embodiments, the halogen is preferably Br, Cl, or F.

As used herein, “haloalkyl” refers to monovalent saturated aliphatic hydrocarbon groups having from 1 to 20 carbon atoms, preferably 1-8 carbon atoms, more preferably 1-6 carbon atoms, wherein at least one hydrogen atom is substituted by a halogen, including but not limited to perhalo groups where all hydrogen atoms are replaced with halogen atoms. The haloalkyl chain can be either straight-chained or branched. Illustrative alkyl groups include trifluoromethyl, trifluoroethyl, trifluoropropyl, trifluorobutyl, and pentafluoroethyl. Similarly, a “haloalkenyl” group refers to a haloalkyl group having one or more double bonds present in the chain, and a “haloalkynyl” group refers to a haloalkyl group having one or more triple bonds present in the chain. Moreover, an “alkylene” linker group refers to a divalent alkyl group, namely (CH₂)ₓ, where x is 1 to 20, preferably 1 to 8, preferably 1 to 6, and more preferably 1 to 3.

The term “haloalkyloxy” refers to O-haloalkyl.

As used herein, “alkoxy” refers to an O-alkyl group having the specified number of carbon atoms.

An “alkylene,” group is an alkyl group, as defined hereinabove, that is positioned between and serves to connect two other chemical groups. Exemplary alkylene groups include, without limitation, methylene, ethylene, propylene, and butylene.

The term “heteroalkyl” refers to an alkyl group, as defined hereinabove, wherein one or more carbon atoms in the chain are replaced by a heteroatom selected from the group consisting of O, S, and N.

As used herein, “hydroxyalkyl” refers to an alkyl group as herein defined substituted with one or more —OH group. Similarly, a “hydroxyalkenyl” group refers to a hydroxyalkyl group having one or more double bonds present in the chain, and a “hydroxyalkynyl” group refers to a hydroxyalkyl group having one or more triple bonds present in the chain. Likewise, a “dihydroxyalkyl” group provides two —OH substituents.

As used herein, “aryl” refers to a substituted or unsubstituted carbocyclic aromatic ring system, either pendent or fused, such as phenyl, naphthyl, anthracenyl, phenanthryl, tetrahydronaphthyl, indane, or biphenyl. A preferred aryl group is phenyl.

An “aralkyl” or “arylalkyl” group comprises an aryl group covalently linked to an alkyl group as defined herein above, either of which may independently be optionally substituted or unsubstituted. An example of an aralkyl group is (C₁-C₆)alkyl(C₆-C₁₀)aryl, including, without limitation, benzyl, phenethyl, and naphthylmethyl. An example of a substituted aralkyl is wherein the alkyl group is substituted with hydroxyalkyl.

As will be appreciated by those skilled in the art, the present disclosure references chemical capture, namely chemical bonds or other interactions that allow atoms to be more stable by, for example, by filling their valence shell of electrons. Covalent bonds, hydrogen bonds, ionic bonds, and van der Waals interactions have varying strengths and characteristics. Covalent bonds, in general, may be defined as a bond formed through the sharing of electrons between two atoms. Covalent bonds are either polar or nonpolar. The nature of the bond is determined by the electronegativities of the atoms in the bond. If their electronegativities are equal, the electrons are equally shared, resulting in a nonpolar covalent bond (e.g. molecular oxygen, O₂). If one of the atoms is significantly more electronegative than the other, the electrons will be shared unequally. This is a polar covalent bond (e.g. nitric oxide, NO). Hydrogen bonds, by name, provide a hydrogen atom weakly shared between two electronegative atoms. Hydrogen bonds are an example of a readily reversible electrostatic interaction. Hydrogen, with a weak positive charge, is attracted to another atom that is weakly negatively charged, for example, fluorine, oxygen, or nitrogen.

The most commonly cited example is water. Ionic bonding provides a bond formed by the electrostatic attraction of two oppositely charged ions. In the context of biochemistry and an aqueous environment, ionic bonds readily separate into their component ions. These oppositely charged ions are still attracted to each other, through electrostatic attraction. Lastly, van der Waals interactions provide the weakest interaction due to the motion of electrons. Electrons are constantly in motion, which leads to temporary patches of negative charge (electrons clustered together) or positive charge (the absence of electrons) on the surface of biological molecules. In the next instant, the patches disappear and reappear somewhere else. Occasionally, there will be a complementary patch on another molecule that allows two molecules to be briefly attracted to each other. As appreciated by those skilled in the art, boron is able to form dative bonds with, for example, oxygen or nitrogen under some circumstances. Dative bonds are usually weaker than covalent bonds. In situations where a boron is covalently bonded to at least one oxygen or nitrogen, and is at the same time datively bonded to an oxygen or nitrogen, respectively, the dative bond and covalent bond between the boron and the two identical heteroatoms can interconvert or be in the form of a resonance hybrid. Additionally, the dative bond may be reversible depending on the chemical structure of the parent compound and the biological environement. For example, reversible dative bonds formed via a boron atom may lead to the formation of hydrolysis products (II) or metabolic oxidation products (III).

Hydrolysis products (II) may be in equilbrium with the parent compound (I). The scope of the present disclosure is intended to capture all forms. A representative example is shown below:

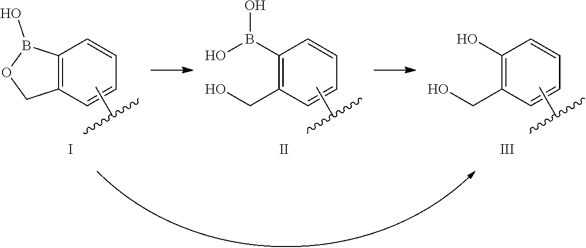

There is potential uncertainty surrounding the exact nature and extent of electron sharing in these situations. The structures supplied are intended to include any and all possible bonding scenarios between boron and the atom to which it is bound.

“Salt counterion”, as used herein, refers to positively charged ions that associate with a compound of the invention when the boron is fully negatively or partially negatively charged. Examples of salt counterions include H⁺, H₃O⁺, ammonium, lithium, potassium, calcium, magnesium and sodium. The compounds comprising a boron bonded to a carbon and three heteroatoms (such as three oxygens described in this section) can optionally contain a fully negatively charged boron or partially negatively charged boron, due to the nature of the dative bond between the boron and one of the oxygens. Due to the negative charge, a positively charged counterion may associate with this compound, thus forming a salt. Examples of positively charged counterions include H⁺, H₃O⁺, calcium, lithium, sodium, ammonium, potassium, magnesium. The salts of these compounds are implicitly contained in descriptions of these compounds. The present invention also encompasses compounds that are poly- or multi-valent species, including, for example, species such as dimers, trimers, tetramers and higher homologs of the compounds of use in the invention or reactive analogues thereof.

As will be appreciated by those skilled in the art, certain aspects of the present disclosure are defined as an adduct, which is intended to describe the complex formed when a compound of the present disclosure binds to a biological molecule, such as a protein.

Similarly, certain aspects of the present disclosure are defined in terms of a warhead, which is intended to describe a functional group that offers chemical capture of a targeted amino acid residue on a target protein, including formation of covalent bonds with one or more targeted amino acids

As used herein, “cycloalkyl” refers to a saturated, an unsaturated or a partially saturated hydrocarbon ring, containing from 3 to 15 ring atoms. Illustrative cycloalkyl groups include cyclopropyl, cyclobutyl, cyclopentyl, cyclohexyl, as well as partially saturated versions thereof, such as cyclohexenyl, and cyclohexadienyl. Moreover, bridged rings, such as adamantane, are included within the definition of “cycloalkyl.”

As used herein, the term “heterocyclyl” refers to an unsaturated or partially saturated hydrocarbon ring, containing from 3 to 15 ring atoms, wherein one or more carbon atom is replaced with a heteroatom selected from B, O, N, S, or Si, where each N, S, or Si may be oxidized, and where each N may be quarternized. A heterocyclyl group may be attached to the remainder of the molecule through a heteroatom. Heterocyclyl does not include heteroaryl. Examples include, but are not limited to, aziridine, oxirane, thiirance, azetidine, oxetane, thietane, pyrrolidine, pyrazolidine, imidazolidine, tetrahydrofuran, tetrahydrothiophene, piperidine, piperazine, tetrahydropyran, thiane, morpholine, thiomorpholine, pyrrolizidine, indoline, decahydroquinoline, tetrahydroquinoline, and azaadamantane.

The term “heterocyclylalkyl” refers to a heterocyclyl group as defined herein covalently linked to an alkyl group as defined hereinabove wherein the radical is on the alkyl group, wherein the alkyl group of the heterocyclylalkyl may be optionally substituted.

As used herein, the term “heteroaryl” or “heteroaromatic” refers to aromatic ring groups having 5 to 14 ring atoms selected from carbon and at least one (typically 1-4, more typically 1 or 2) heteroatom (e.g., boron, oxygen, nitrogen, sulfur, or silicon). They include monocyclic rings and polycyclic rings in which a monocyclic heteroaromatic ring is fused to one or more other carbocyclic aromatic or heteroaromatic rings. Examples of monocyclic heteroaryl groups include, but are not limited to, furanyl (e.g., 2-furanyl, 3-furanyl), imidazolyl (e.g., N-imidazolyl, 2-imidazolyl, 4-imidazolyl, 5-imidazolyl), isoxazolyl (e.g., 3-isoxazolyl, 4-isoxazolyl, 5-isoxazolyl), oxadiazolyl (e.g., 2-oxadiazolyl, 5-oxadiazolyl), oxazolyl (e.g., 2-oxazolyl, 4-oxazolyl, 5-oxazolyl), pyrazolyl (e.g., 3-pyrazolyl, 4-pyrazolyl), pyrrolyl (e.g., 1-pyrrolyl, 2-pyrrolyl, 3-pyrrolyl), pyridyl (e.g., 2-pyridyl, 3-pyridyl, 4-pyridyl), pyrimidinyl (e.g., 2-pyrimidinyl, 4-pyrimidinyl, 5-pyrimidinyl), pyridazinyl (e.g., 3-pyridazinyl), thiazolyl (e.g., 2-thiazolyl, 4-thiazolyl, 5-thiazolyl), triazolyl (e.g., 2-triazolyl, 5-triazolyl), tetrazolyl (e.g., tetrazolyl) and thienyl (e.g., 2-thienyl, 3-thienyl. Examples of monocyclic six-membered nitrogen-containing heteroaryl groups include pyrimidinyl, pyridinyl and pyridazinyl. Examples of polycyclic aromatic heteroaryl groups include carbazolyl, benzimidazolyl, benzothienyl, benzofuranyl, indolyl, quinolinyl, benzotriazolyl, benzothiazolyl, benzoxazolyl, benzimidazolyl, isoquinolinyl, indolyl, isoindolyl, acridinyl, or benzisoxazolyl.

The terms “arylalkyl,” “heteroarylalkyl,” and “heterocyclylalkyl” refers to those radicals in which an aryl, heteroaryl, or heterocyclyl group is linked through an alkyl group. Examples includes benzyl, phenethyl, pyridylmethyl, and the like. The terms also include alkyl linking groups in which a carbon atom, for example, a methylene group, has been replaced by, for example, an oxygen atom. Examples include phenoxymethyl, pyrid-2-yloxymethyl, 3-(naphth-1-yloxy)propyl, and the like. Similarly, the term “benzyl” as used herein is a radical in which a phenyl group is attached to a CH₂ group, thus, a CH₂Ph group. Benzyl groups may be substituted or unsubstituted. The term substituted benzyl refers to radicals in which the phenyl group or CH₂ contains one or more substituents. In one embodiment, the phenyl group may have 1 to 5 substituents, or in another embodiment 2 to 3 substituents.

A “heteroarylalkyl” group comprises a heteroaryl group covalently linked to an alkyl group, wherein the radical is on the alkyl group, either of which is independently optionally substituted or unsubstituted. Examples of heteroarylalkyl groups include a heteroaryl group having 5, 6, 9, or 10 ring atoms bonded to a C1-C6 alkyl group. Examples of heteroaralkyl groups include pyridylmethyl, pyridylethyl, pyrrolylmethyl, pyrrolylethyl, imidazolylmethyl, imidazolylethyl, thiazolylmethyl, thiazolylethyl, benzimidazolylmethyl, benzimidazolylethyl quinazolinylmethyl, quinolinylmethyl, quinolinylethyl, benzofuranylmethyl, indolinylethyl isoquinolinylmethyl, isoinodylmethyl, cinnolinylmethyl, and benzothiophenylethyl. Specifically excluded from the scope of this term are compounds having adjacent annular 0 and/or S atoms.

As used herein “optionally substituted” refers to a substitution of a hydrogen atom, which would otherwise be present for the substituent. When discussing ring systems, the optional substitution is typically with 1, 2, or 3 substituents replacing the normally-present hydrogen. When referencing straight and branched moieties, however, the number of substitutions may be more, occurring wherever hydrogen is present. The substitutions may be the same or different.

Illustrative substituents, which with multiple substituents can be the same or different, include deuterium, halogen, haloalkyl, R′, OR′, OH, SH, SR′, NO₂, CN, C(O)R′, NH₂, C(O)OR′, OC(O)R′, CON(R′)₂, OC(O)N(R′)₂, NH₂, NHR′, N(R′)₂, NHCOR′, NHCOH, NHCONH₂, NHCONHR′, NHCON(R′)₂, NRCOR′, NRCOH, NHCO₂H, NHCO₂R′, NHC(S)NH₂, NHC(S)NHR′, NHC(S)N(R′)₂, CO₂R′, CO₂H, CHO, CONH₂, CONHR′, CON(R)₂, S(O)₂H, S(O)₂R′, SO₂NH₂, S(O)H, S(O)R′, SO₂NHR′, SO₂N(R′)₂, NHS(O)₂H, NR′S(O)₂H, NHS(O)₂R′, NR′S(O)₂R′, Si(R′)₃, where each of the preceding may be linked through a divalent alkylene linker, (CH₂)ₓ, where x is 1, 2, or 3. In embodiments where a saturated carbon atom is optionally substituted with one or more substituent groups, the substituents may be the same or different and also include ═O, ═S, ═NNHR′, ═NNH₂, ═NN(R′)₂, ═N—OR′, ═N—OH, ═NNHCOR′, ═NNHCOH, ═NNHCO₂R′, ═NNHCO₂H, ═NNHSO₂R′, ═NNHSO₂H, ═N—CN, ═NH, or ═NR′. For each of the preceding, each may be linked through an alkylene linker, (CH₂)ₓ, where x is 1, 2, or 3, Each occurrence of R′ is the same or different and represents hydrogen, alkyl, alkenyl, alkynyl, cycloalkyl, aryl, heterocyclyl, or heteroaryl, or when two R′ are each attached to a nitrogen atom, they may form a saturated or unsaturated heterocyclic ring containing from 4 to 6 ring atoms.

In some embodiments of the present disclosure, a heteroatom may be a boron (B) atom.

The scope of the present disclosure includes isotopic versions of the compounds herein disclosed. As an example, one strategy to slow the CYP-mediated metabolism of a drug or to reduce the formation of undesirable metabolites includes an attempt to replace one or more hydrogen atoms with deuterium atoms. Deuterium is a safe, stable, non-radioactive isotope of hydrogen. Compared to hydrogen, deuterium forms stronger bonds with carbon. In select cases, the increased bond strength imparted by deuterium can positively impact the ADME properties of a drug, creating the potential for improved drug efficacy, safety, and/or tolerability. At the same time, because the size and shape of deuterium are essentially identical to those of hydrogen, replacement of hydrogen by deuterium would not be expected to affect the biochemical potency and selectivity of the drug as compared to the original chemical entity that contains only hydrogen.

Over the past 35 years, the effects of deuterium substitution on the rate of metabolism have been reported for a very small percentage of approved drugs (see, e.g., Blake, M I et al, J Pharm Sci, 1975, 64:367-91; Foster, A B, Adv Drug Res 1985, 14:1-40 (“Foster”); Kushner, D J et al, Can J Physiol Pharmacol 1999, 79-88; Fisher, M B et al, Curr Opin Drug Discov Devel, 2006, 9:101-09 (“Fisher”)).

The results have been variable and unpredictable. For some compounds deuteration caused decreased metabolic clearance in vivo. For others, there was no change in metabolism. Still others demonstrated increased metabolic clearance. The variability in deuterium effects has also led experts to question or dismiss deuterium modification as a viable drug design strategy for inhibiting adverse metabolism (see Foster at p. 35 and Fisher at p. 101).

The effects of deuterium modification on a drug's metabolic properties are not predictable even when deuterium atoms are incorporated at known sites of metabolism. Only by actually preparing and testing a deuterated drug can one determine if and how the rate of metabolism will differ from that of its non-deuterated counterpart. See, for example, Fukuto et al. (J. Med. Chem. 1991, 34, 2871-76). Many drugs have multiple sites where metabolism is possible. The site(s) where deuterium substitution is required and the extent of deuteration necessary to see an effect on metabolism, if any, will be different for each drug.

In the compounds of this disclosure, any atom designated specifically as “H” or “hydrogen”, that position is understood to have hydrogen at its natural abundance isotopic composition. Also unless otherwise stated, that position may be designated specifically as “D” or “deuterium”, where the position is understood to have deuterium at an abundance that is at least 3340 times greater than the natural abundance of deuterium, which is 0.015% (i.e., at least 50.1% incorporation of deuterium).

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

A compound represented by a particular chemical structure containing one or more deuterium atoms, will also contain lesser amounts of isotopologues having hydrogen atoms at one or more of the potential deuterium positions. The relative amount of such isotopologues in a compound of this disclosure will depend upon a number of factors including the isotopic purity of deuterated reagents used to make the compound and the efficiency of incorporation of deuterium in the various synthesis steps used to prepare the compound. As set forth above, the relative amount of such isotopologues will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

As used herein, “an effective amount” of a compound is an amount that is sufficient to negatively modulate or inhibit the activity of PI3K or a mutant thereof. Such amount may be administered as a single dosage or may be administered according to a regimen, whereby it is effective.

As used herein, a “therapeutically effective amount” of a compound is an amount that is sufficient to ameliorate, or in some manner reduce a symptom or stop or reverse progression of a condition, or negatively modulate or inhibit the activity of PI3K or a mutant thereof. Such amount may be administered as a single dosage or may be administered according to a regimen, whereby it is effective.

As used herein, treatment means any manner in which the symptoms or pathology of a condition, disorder or disease are ameliorated or otherwise beneficially altered. Treatment also encompasses any pharmaceutical use of the compositions herein.

As used herein, amelioration of the symptoms of a particular disorder by administration of a particular pharmaceutical composition refers to any lessening, whether permanent or temporary, lasting or transient that can be attributed to or associated with administration of the composition.

As used herein, the term “about” when used to modify a numerically defined parameter (e.g., the dose of the PI3K inhibitor detailed herein or a pharmaceutically acceptable salt thereof, or the length of treatment time described herein) means that the parameter may vary by as much as 10% below or above the stated numerical value for that parameter. For example, a dose of about 5 mg/kg may vary between 4.5 mg/kg and 5.5 mg/kg. “About” when used at the beginning of a listing of parameters is meant to modify each parameter. For example, about 0.5 mg, 0.75 mg or 1.0 mg means about 0.5 mg, about 0.75 mg or about 1.0 mg. Likewise, about 5% or more, 10% or more, 15% or more, 20% or more, and 25% or more means about 5% or more, about 10% or more, about 15% or more, about 20% or more, and about 25% or more.

As used herein, a salt refers to any salt of a compound disclosed herein which retains its biological properties and which is not toxic or otherwise undesirable for pharmaceutical use.

Such salts may be derived from a variety of organic and inorganic counter-ions known in the art. Such salts include acid addition salts formed with organic or inorganic acids such as hydrochloric, hydrobromic, sulfuric, nitric, phosphoric, sulfamic, acetic, trifluoroacetic, trichioroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid, and like acids.

Salts further include, by way of example only, salts of non-toxic organic or inorganic acids, such as halides, such as, chloride and bromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate, and the like.

Examples of inorganic bases that may be used to form base addition salts include, but are not limited to, metal hydroxides, such as lithium hydroxide, sodium hydroxide, and potassium hydroxide; metal amides, such as lithium amide and sodium amide; metal carbonates, such as lithium carbonate, sodium carbonate, and potassium carbonate; and ammonium bases such as ammonium hydroxide and ammonium carbonate.

Examples of organic bases that may be used to form base addition salts include, but are not limited to, metal alkoxides, such as lithium, sodium, and potassium alkoxides including lithium methoxide, sodium methoxide, potassium methoxide, lithium ethoxide, sodium ethoxide, potassium ethoxide, and potassium tert-butoxide; quaternary ammonium hydroxides, such as choline hydroxide; and amines including, but not limited to, aliphatic amines (i.e., alkylamines, alkenylamines, alkynylamines, and alicyclic amines), heterocyclic amines, arylamines, heteroarylamines, basic amino acids, amino sugars, and polyamines. In some embodiments, salt forms may include lithium, sodium, potassium, and amine salts.

The base may be a quaternary ammonium hydroxide, wherein one or more of the alkyl groups of the quaternary ammonium ion are optionally substituted with one or more suitable substituents. Preferably, at least one alkyl group is substituted with one or more hydroxyl groups. Non-limiting examples of quaternary ammonium hydroxides that may be used in accordance with the present disclosure include choline hydroxide, trimethylethylammonium hydroxide, tetramethylammonium hydroxide, and is preferably choline hydroxide. An alkylamine base may be substituted or unsubstituted. Non-limiting examples of unsubstituted alkylamine bases that may be used in accordance with the present disclosure include methylamine, ethylamine, diethylamine, and triethylamine. A substituted alkylamine base may be substituted with one or more hydroxyl groups, and preferably one to three hydroxyl groups. Non-limiting examples of substituted alkylamine bases that may be used in accordance with the present disclosure include 2-(diethylamino)ethanol, N,N-dimethylethanolamine (deanol), tromethamine, ethanolamine, and diolamine.

In certain cases, the depicted substituents may contribute to optical isomers and/or stereoisomerism. Compounds having the same molecular formula but differing in the nature or sequence of bonding of their atoms or in the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers”. When a compound has an asymmetric center, for example when it is bonded to four different groups, a pair of enantiomers is possible. A molecule with at least one stereocenter may be characterized by the absolute configuration of its asymmetric center and is designated (R) or (S) according to the rules of Cahn and Prelog (Cahn et al., 1966, Angew. Chem. 78: 413-447, Angew. Chem., Int. Ed. Engl. 5: 385-414 (errata: Angew. Chem., Int. Ed. Engl. 5:511); Prelog and Helmchen, 1982, Angew. Chem. 94: 614-631, Angew. Chem. Internat. Ed. Eng. 21: 567-583; Mata and Lobo, 1993, Tetrahedron: Asymmetry 4: 657-668) or may be characterized by the manner in which the molecule rotates the plane of polarized light and is designated dextrorotatory or levorotatory (namely, as (+)- or (−)-isomers, respectively). A chiral compound may exist as either an individual enantiomer or as a mixture thereof. A mixture containing equal proportions of enantiomers is called a “racemic mixture”.

In certain embodiments, the compounds disclosed herein may possess one or more asymmetric centers, and such compounds may therefore be produced as a racemic mixture, an enantiomerically enriched mixture, or as an individual enantiomer. Unless indicated otherwise, for example by designation of stereochemistry at any position of a formula, the description or naming of a particular compound in the specification and claims is intended to include both individual enantiomers and mixtures, racemic or otherwise, thereof. Methods for determination of stereochemistry and separation of stereoisomers are well-known in the art.

In certain embodiments, the compounds disclosed herein are “stereochemically pure”. A stereochemically pure compound has a level of stereochemical purity that would be recognized as “pure” by those of skill in the art. Of course, this level of purity may be less than 100%. In certain embodiments, “stereochemically pure” designates a compound that is substantially free, i.e. at least about 85% or more, of alternate isomers. In particular embodiments, the compound is at least about 85%, about 90%, about 91%, about 92%, about 93%, about 94%, about 95%, about 96%, about 97%, about 98%, about 99%, about 99.5% or about 99.9% free of other isomers.

All isomeric forms (especially all regio- and stereoisomeric forms, e.g. all chiral, enantiomeric, diastereomeric, racemic forms, tautomeric and all geometric isomeric forms, as well as atropisomers, (including interconverting atropisomerism) of a compound of the present description are intended within this invention, unless the specific isomer form is specifically indicated. Obviously, the isomer which is pharmacologically most effective and most free from side effects is preferred.

As used herein, the terms “subject” and “patient” may be used interchangeably herein. In one embodiment, the subject is a human. In one embodiment, the subject is a companion animal such as a dog or cat. In a further embodiment, the subject is an animal such as a sheep, cow, horse, goat, fish, pig, or domestic fowl (e.g., chicken, turkey, duck, or goose). In another embodiment, the subject is a primate such as a monkey such as a cynomolgous monkey or a chimpanzee.

In addition, a pharmaceutically acceptable prodrug of the compound represented by the formulae is also included in the present disclosure. The pharmaceutically acceptable prodrug refers to a compound having a group which may be converted into an amino group, a hydroxyl group, a carboxyl group, or the like, by solvolysis or under a physiological condition. Examples of the groups forming the prodrug include those as described in Prog. Med., 5, 2157-2161 (1985) or “Pharmaceutical Research and Development” (Hirokawa Publishing Company, 1990), vol. 7, Drug Design, 163-198. The term prodrug is used throughout the specification to describe any pharmaceutically acceptable form of a compound which, upon administration to a patient, provides the active compound. Pharmaceutically acceptable prodrugs refer to a compound that is metabolized, for example hydrolyzed or oxidized, in the host to form the compound of the present disclosure. Typical examples of prodrugs include compounds that have biologically labile protecting groups on a functional moiety of the active compound. Prodrugs include compounds that may be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound.

The present disclosure includes all pharmaceutically acceptable isotopically-labelled compounds of the disclosure wherein one or more atoms are replaced by atoms having the same atomic number, but an atomic mass or mass number different from the atomic mass or mass number usually found in nature.

Examples of isotopes suitable for inclusion in the compounds of the disclosure include isotopes of hydrogen, such as ²H and ³H, carbon, such as ¹¹C, ¹³C and ¹⁴C, chlorine, boron, such as ¹⁰B and ¹¹B, such as ³⁶Cl, fluorine, such as ⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S. Certain isotopically-labelled compounds of the disclosure, such as those incorporating a radioactive isotope, may be useful in drug or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁰B, ¹⁸F, ¹⁵O and ¹³N, may be useful in Positron Emission Topography (PET) studies for examining substrate receptor occupancy. Reference is made to BNCT or boron neutron capture therapy. Isotopically-labeled compounds of the disclosure may generally be prepared by conventional techniques known to those skilled in the art or by processes analogous to those described in the accompanying Examples using an appropriate isotopically-labeled reagent in place of the non-labeled reagent previously employed.

### Compositions and Methods of Administration

The compounds of the present disclosure used in the methods disclosed herein may be administered in certain embodiments using pharmaceutical compositions including at least one compound, if appropriate in the salt form, either used alone or in the form of a combination with one or more compatible and pharmaceutically acceptable carriers, such as diluents or adjuvants, or with another agent. There are provided compositions which comprise a derivative of a compound of the present disclosure or a salt thereof, and an acceptable excipient, carrier or diluent. The composition may also be in a variety of forms which include, but are not limited to, oral formulations, injectable formulations, and topical, dermal or subdermal formulations.

The composition may be in a form suitable for oral use, for example, as dietary supplements, troches, lozenges, chewables, tablets, hard or soft capsules, emulsions, aqueous or oily suspensions, aqueous or oily solutions, dispersible powders or granules, syrups, or elixirs. Compositions intended for oral use may be prepared according to any method known in the art for the manufacture of pharmaceutical compositions and such compositions may contain one or more agents selected from the group consisting of sweetening agents, bittering agents, flavoring agents, coloring agents and preserving agents in order to provide elegant and palatable preparations.

Lozenges are solid compositions containing one or more active ingredients intended to dissolve or disintegrate slowly in the oral cavity by passive incubation in the oral cavity, or actively by sucking or chewing. They may be used for systemic effect if the drug is absorbed through the buccal or esophageal lining or is swallowed. In particular, soft lozenges may be chewed or allowed to dissolve slowly in the mouth. These dosage forms have the advantage of being flavored and thus easy to administer to both human and animal patients; have formulas that are easy to change and may be patient specific; may deliver accurate amounts of the active ingredient to the oral cavity and digestive system; and allow for the drug to remain in contact with the oral or esophageal cavity for an extended period of time.

Tablets may contain the active ingredient in admixture with non-toxic, pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period.

Formulations for oral use may be hard gelatin capsules, wherein the active ingredient is mixed with an inert solid diluent, for example, calcium carbonate, calcium phosphate or kaolin. Capsules may also be soft gelatin capsules, wherein the active ingredient is mixed with water or miscible solvents such as propylene glycol, PEGs and ethanol, or an oil medium, for example, peanut oil, liquid paraffin, or olive oil.

The compositions may also be in the form of oil-in-water or water-in-oil emulsions. The oily phase may be a vegetable oil, for example, olive oil or arachis oil, or a mineral oil, for example, liquid paraffin or mixtures of these. Suitable emulsifying agents may be naturally-occurring phosphatides, for example, soy bean, lecithin, and esters or partial esters derived from fatty acids and hexitol anhydrides, for example, sorbitan monoleate, and condensation products of the said partial esters with ethylene oxide, for example, polyoxyethylene sorbitan monooleate. The emulsions may also contain sweetening agents, bittering agents, flavoring agents, and preservatives.

In one embodiment of the formulation, the composition is in the form of a microemulsion. Microemulsions are well suited as the liquid carrier vehicle. Microemulsions are quaternary systems comprising an aqueous phase, an oily phase, a surfactant and a cosurfactant. They are translucent and isotropic liquids. Microemulsions are composed of stable dispersions of microdroplets of the aqueous phase in the oily phase or conversely of microdroplets of the oily phase in the aqueous phase. The size of these microdroplets is less than 200 nm (1000 to 100,000 nm for emulsions). The interfacial film is composed of an alternation of surface-active (SA) and co-surface-active (Co-SA) molecules which, by lowering the interfacial tension, allows the microemulsion to be formed spontaneously. In one embodiment of the oily phase, the oily phase may be formed from mineral or vegetable oils, from unsaturated polyglycosylated glycerides or from triglycerides, or alternatively from mixtures of such compounds. In one embodiment of the oily phase, the oily phase comprises of triglycerides; in another embodiment of the oily phase, the triglycerides are medium-chain triglycerides, for example, C₈-C₁₀ caprylic/capric triglyceride. In another embodiment, the oily phase will represent a % v/v range selected from the group consisting of about 2 to about 15%; about 7 to about 10%; and about 8 to about 9% v/v of the microemulsion. The aqueous phase includes, for example, water or glycol derivatives, such as propylene glycol, glycol ethers, polyethylene glycols or glycerol. In one embodiment of the glycol derivatives, the glycol is selected from the group consisting of propylene glycol, diethylene glycol monoethyl ether, dipropylene glycol monoethyl ether and mixtures thereof. Generally, the aqueous phase will represent a proportion from about 1 to about 4% v/v in the microemulsion. Surfactants for the microemulsion include diethylene glycol monoethyl ether, dipropylene glycol monomethyl ether, polyglycolyzed C₈-C₁₀ glycerides or polyglyceryl-6 dioleate. In addition to these surfactants, the cosurfactants include short-chain alcohols, such as ethanol and propanol. Some compounds are common to the three components discussed above, for example, aqueous phase, surfactant and cosurfactant. However, it is well within the skill level of the practitioner to use different compounds for each component of the same formulation. In one embodiment, for example, for the amount of surfactant/cosurfactant, the cosurfactant to surfactant ratio may be from about 1/10 to about 1/2. In another embodiment for the amount of cosurfactant, there will be from about 25 to about 75% v/v of surfactant and from about 10 to about 55% v/v of cosurfactant in the microemulsion.

Oily suspensions may be formulated by suspending the active ingredient in a vegetable oil, for example, atachis oil, olive oil, sesame oil or coconut oil, or in mineral oil such as liquid paraffin. The oily suspensions may contain a thickening agent, for example, beeswax, hard paraffin or cetyl alcohol. Sweetening agents such as sucrose, saccharin or aspartame, bittering agents, and flavoring agents may be added to provide a palatable oral preparation. These compositions may be preserved by the addition of an anti-oxidant such as ascorbic acid, or other known preservatives.

Aqueous suspensions may contain the active material in admixture with excipients suitable for the manufacture of aqueous suspensions. Such excipients are suspending agents, for example, sodium carboxymethylcellulose, methylcellulose, hydroxypropylmethylcellulose, sodium alginate, polvinylpyrrolidone, gum tragacanth and gum acacia; dispersing or wetting agents may be a naturally-occuring phosphatide, for example, lecithin, or condensation products of an alkylene oxide with fatty acids, for example, polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide, with partial esters derived from fatty acids and hexitol anhydrides, for example, polyethylene sorbitan monooleate. The aqueous suspensions may also contain one or more preservatives, for example, ethyl, or n-propyl, p-hydroxybenzoate, one or more coloring agents, one or more flavoring agents, and one or more sweetening agents and/or bittering agents, such as those set forth herein.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, bittering, flavoring and coloring agents, may also be present.

As used herein, the term “dispersion” refers to a disperse system in which one substance, the dispersed phase, is distributed, in discrete units, throughout a second substance (the continuous phase or vehicle). The size of the dispersed phase can vary considerably (e.g. colloidal particles of nanometer dimension, to multiple microns in size). In general, the dispersed phases can be solids, liquids, or gases.

In the case of a solid dispersion, the dispersed and continuous phases are both solids. In pharmaceutical applications, a solid dispersion can include a crystalline drug (dispersed phase) in an amorphous polymer (continuous phase); or alternatively, an amorphous drug (dispersed phase) in an amorphous polymer (continuous phase). In some embodiments, a solid dispersion includes the polymer constituting the dispersed phase, and the drug constitute the continuous phase. In other embodiments, a solid dispersion includes the drug constituting the dispersed phase, and the polymer constituting the continuous phase. An amorphous solid dispersion (ASD) refers to an amorphous active pharmaceutical ingredient stabilized by a polymer matrix to provide enhanced characteristics, including stability, to a solid material having no long range order in the position of its molecules. The moledules in an amorphous solid are generally arranged in a random manner with no well-defined arrangement. Amorphous solids are generally isotropic, i.e. exhibit similar properties in all directions and do not have definite melting points. Amorphous solid dispersions (ASDs) may be used for poorly soluble pharmaceutical compounds. In an ASD, the solubility of the drug substance is improved by disarranging its crystalline lattice to produce a higher energy state of amorphous form (See, Duarte et al., 2015; Elgindy et al., 2011).

Spray drying converts a liquid feed to a dried particulate form. Spray drying generally involves bringing into contact a highly dispersed liquid suspension or solution and a sufficient volume of hot air to promote drying of the liquid droplets. For example, a liquid solution containing a compound of the present disclosure, or a salt thereof and at least one polymer can be sprayed into a current of warm filtered gas that evaporates the solvent and conveys the dried product to a collector. Evaporated solvent and spent gas are removed from the collector and can be sent to a condenser to capture the solvent. For example, commercial spray dryers are manufactured by Buchi Ltd. and Niro (e.g., the PSD line of spray driers manufactured by Niro) (see, US 2004/0105820, US 2003/0144257). Techniques and methods for spray drying may be found in Perry's Chemical Engineering Handbook, 6th Ed., R. H. Perry, D. W. Green & J. O. Maloney, eds.), McGraw-Hill book co. (1984); and Marshall “Atomization and Spray-Drying” 50, Chem. Eng. Prog. Monogr. Series 2 (1954). All three references are incorporated herein in their entirety by reference. Accordingly, compositions comprising compounds of the present disclosure may prepared as spray dry dispersions.

Syrups and elixirs may be formulated with sweetening agents, for example, glycerol, propylene glycol, sorbitol or sucrose. Such formulations may also contain a demulcent, a preservative, flavoring agent(s) and coloring agent(s).

The compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example, as a solution in 1,3-butane diol. Among the acceptable vehicles and solvents that may be employed are water, Ringer's solution and isotonic sodium chloride solution. Cosolvents such as ethanol, propylene glycol or polyethylene glycols may also be used. Preservatives, such as phenol or benzyl alcohol, may be used.

In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

Topical, dermal and subdermal formulations may include emulsions, creams, ointments, gels or pastes.

Organic solvents that may be used in the disclosure include but are not limited to: acetyltributyl citrate, fatty acid esters such as the dimethyl ester, diisobutyl adipate, acetone, acetonitrile, benzyl alcohol, butyl diglycol, dimethylacetamide, dimethylformamide, dipropylene glycol n-butyl ether, ethanol, isopropanol, methanol, ethylene glycol monoethyl ether, ethylene glycol monomethyl ether, monomethylacetamide, dipropylene glycol monomethyl ether, liquid polyoxyethylene glycols, propylene glycol, 2-pyrrolidone (e.g. N-methylpyrrolidone), diethylene glycol monoethyl ether, ethylene glycol and diethyl phthalate, or a mixture of at least two of these solvents.

As vehicle or diluent, compositions of the present disclosure may include plant oils such as, but not limited to soybean oil, groundnut oil, castor oil, corn oil, cotton oil, olive oil, grape seed oil, sunflower oil, etc.; mineral oils such as, but not limited to, petrolatum, paraffin, silicone, etc.; aliphatic or cyclic hydrocarbons or alternatively, for example, medium-chain (such as C₈-C₁₂) triglycerides.

Dosage forms may contain from about 0.5 mg to about 5 g of an active agent.

In one embodiment of the disclosure, the active agent is present in the formulation at a concentration of about 0.05 to 10% weight/volume.

A compound of the present disclosure may be employed as such or in the form of their preparations or formulations as combinations.

These one or more additional active agents may be administered as part of the same or separate dosage forms, via the same or different routes of administration, and on the same or different administration schedules according to standard pharmaceutical practice known to one skilled in the art.

The pharmaceutical preparation comprising the compounds of the present disclosure for delivery to a human or other mammal, is preferably in unit dosage form, in which the preparation is subdivided into unit doses containing an appropriate quantity of the active component. The unit dosage form may be a packaged preparation containing discrete quantities of the preparation, such as packaged tablets, capsules, and powders in vials or ampoules. Also, the unit dosage form may be a capsule, tablet or lozenge itself, or it may be an appropriate number of any of these in packaged form.

The quantity of active component in a unit dose preparation may be varied or adjusted from about 0.1 mg to about 1000 mg, according to the particular application and the potency of the active component.

The composition may, if desired, also contain other compatible therapeutic agents.

In therapeutic use for the treatment or alleviation of one or more symptoms for one or more diseases or disorders, such as cancer, in a human or other mammal, the compounds utilized in the method of treatment are administered at an initial dosage of about 0.1 mg/kg to about 1,000 mg/kg per interval, about 0.1 mg/kg to about 500 mg/kg per interval, about 0.1 mg/kg to about 100 mg/kg per interval, about 0.1 mg/kg to about 50.0 mg/kg per interval, about 0.1 mg/kg to about 10.0 mg/kg per interval, about 0.1 mg/kg to about 5.0 mg/kg per interval, about 0.1 mg/kg to about 2.5 mg/kg per interval, about 0.1 mg/kg to about 2.0 mg/kg per interval, about 0.1 mg/kg to about 1.0 mg/kg per interval, about 0.4 mg/kg to about 1.0 mg/kg per interval, or about 0.4 mg/kg to about 0.6 mg/kg per interval. Preferred intervals may be daily, twice-daily, thrice-daily, weekly, bi-weekly, monthly, quarterly, semi-annually, or annually.

The dosages may be varied depending on the requirements of the patient, for example, the size of the human or mammal being treated, the severity of the condition being treated, the route of administration, and the potency of the compound(s) being used. Determination of the proper dosage and route of administration for a particular situation is within the skill of the practitioner. Generally, the treatment will be initiated with smaller dosages, which are less than the optimum dose of the compound, which may be increased in small increments until the optimum effect under the particular circumstances of the condition is reached. For convenience, the total daily dosage may be divided and administered in portions during the day if desired.

In therapeutic use, the compounds of the present dislcosure are useful in manufacture of a medicament for a method of the treating any indication where inhibition of PI3K or a mutant variant thereof would be desirable.

One embodiment of the present disclosure provides the compounds of the present disclosure incorporated in a proteolysis targeting chimera (PROTAC), namely a heterobifunctional molecule comprising two active domains and a linker. A PROTAC may include an E3 ubiquitin ligase targeting moiety and a compound of the present disclosure, namely a targeting warhead to bind a target protein meant for degradation.

In yet another aspect, the disclosure provides for methods for inhibiting PI3K, or a mutant thereof, activity in a cell, comprising contacting the cell in which inhibition of PI3K, or a mutant thereof, activity is desired with an effective amount of a compound of the present disclosure, pharmaceutically acceptable salts thereof, or pharmaceutical compositions containing the compound or pharmaceutically acceptable salt thereof. In one embodiment, the contacting is in vitro. In one embodiment, the contacting is in vivo.

As used herein, the term “contacting” refers to the bringing together of indicated moieties in an in vitro system or an in vivo system. For example, “contacting” a PI3K, or a mutant thereof, with a compound provided herein includes the administration of a compound provided herein to an individual or patient, such as a human, having PI3K, or a mutant thereof, as well as, for example, introducing a compound provided herein into a sample containing a cellular or purified preparation containing the PI3K, or a mutant thereof.

In one embodiment, a cell in which inhibition of PI3K, or a mutant thereof, activity is desired is contacted with an effective amount of a compound of the present disclosure to negatively modulate the activity. In other embodiments, a therapeutically effective amount of pharmaceutically acceptable salt or pharmaceutical compositions containing the compound of the present disclosure may be used.

By negatively modulating the activity of PI3K, or a mutant thereof, the methods described herein are designed to inhibit undesired cellular proliferation resulting from enhanced PI3K, or a mutant thereof, activity within the cell. The cells may be contacted in a single dose or multiple doses in accordance with a particular treatment regimen to effect the desired negative modulation of PI3K, or a mutant thereof. The degree of modification of PI3K, or a mutant thereof, may be monitored in vitro using well known methods, including those described below. In addition, the inhibitory activity of exemplary compounds in cells may be monitored, for example, by measuring the inhibition of PI3K, or a mutant thereof, to assess the effectiveness of treatment and dosages may be adjusted accordingly by the attending medical practitioner.

In another aspect, methods of treating cancer in a patient in need thereof, comprising administering to said patient a therapeutically effective amount of a compound of the present disclosure, pharmaceutically acceptable salts thereof or pharmaceutical compositions comprising the compound or pharmaceutically acceptable salts thereof are provided.

The compositions and methods provided herein may be used for the treatment of a PI3K-associated cancer (or mutant variant thereof) in a patient in need thereof, comprising administering to said patient a therapeutically effective amount of a compound of the present disclosure, pharmaceutically acceptable salts thereof or pharmaceutical compositions comprising the compound or pharmaceutically acceptable salts thereof are provided. In one embodiment, the PI3K-associated, or mutant variant thereof, is cancer.

The compositions and methods provided herein may be used for the treatment of a wide variety of cancers including tumors such as lung, prostate, breast, brain, skin, cervical carcinomas, testicular carcinomas, etc. More particularly, cancers that may be treated by the compositions and methods of the disclosure include, but are not limited, to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is selected from breast cancer, colorectal cancer, uterine cancer, bladder cancer, lung cancer, giloma, head and neck cancer, and other solid tumors. In some embodiments, the cancer is breast cancer.

The concentration and route of administration to the patient will vary depending on the cancer to be treated. The compounds, pharmaceutically acceptable salts thereof and pharmaceutical compositions comprising such compounds and salts also may be co-administered with other anti-neoplastic compounds, e.g., chemotherapy, or used in combination with other treatments, such as radiation or surgical intervention, either as an adjuvant prior to surgery or post-operatively.

In another aspect the disease/condition/cancer to be treated/prevented as herein (above and below) defined is selected from the group consisting of pancreatic cancer, colorectal cancer, lung cancer, multiple myeloma, melanoma, uterine cancer, endometrial cancer, thyroid cancer, acute myeloid leukemia, bladder cancer, urothelial cancer, gastric cancer, cervical cancer, head and neck squamous cell carcinoma, diffuse large B cell lymphoma, esophageal cancer, chronic lymphocytic leukemia, hepatocellular cancer, breast cancer, ovarian cancer, prostate cancer, glioblastoma, renal cancer and sarcomas, salivary gland cancers and urinary tract cancers.

The compounds of the disclosure may be used on their own or in combination with one or several other pharmacologically active substances such as state-of-the-art or standard-of-care compounds, such as e.g. cell proliferation inhibitors, anti-angiogenic substances, steroids or immune modulators/checkpoint inhibitors, and the like. As an SHP2 (Src homology-2 domain-containing protein tyrosine phosphatase-2) is a non-receptor protein tyrosine phosphatase that removes tyrosine phosphorylation. Functionally, SHP2 serves as an important hub to connect several intracellular oncogenic signaling pathways, such as Jak/STAT, PI3K/AKT, RAS/Raf/MAPK, and PD-1/PD-L1 pathways. Mutations and/or overexpression of SHP2 has been associated with genetic developmental diseases and cancers.

Pharmacologically active substances which may be administered in combination with the compounds according to the disclosure, include, without being restricted thereto, hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxyprogesterone, octreotide), aromatase inhibitors (e.g. anastrozole, letrozole, liarozole, vorozole, exemestane, atamestane), LHRH agonists and antagonists (e.g. goserelin acetate, luprolide), inhibitors of growth factors and/or of their corresponding receptors (growth factors such as for example platelet derived growth factor (PDGF), fibroblast growth factor (FGF), vascular endothelial growth factor (VEGF), epidermal growth factor (EGF), insuline-like growth factors (IGF), human epidermal growth factor (HER, e.g. HER2, HER3, HER4), and hepatocyte growth factor (HGF) and/or their corresponding receptors), inhibitors are for example (anti-)growth factor antibodies, (anti) growth factor receptor antibodies and tyrosine kinase inhibitors, such as for example cetuximab, gefitinib, afatinib, nintedanib, imatinib, lapatinib, bosutinib, bevacizumab and trastuzumab); antimetabolites (e.g. antifolates such as methotrexate, raltitrexed, pyrimidine analogues such as 5-fluorouracil (5-FU), ribonucleoside and deoxyribonucleoside analogues, capecitabine and gemcitabine, purine and adenosine analogues such as mercaptopurine, thioguanine, cladribine and pentostatin, cytarabine (ara C), fludarabine); antitumour antibiotics (e.g. anthracyclins such as doxorubicin, doxil (pegylated liposomal doxorubicin hydrochloride, myocet (non-pegylated liposomal doxorubicin), daunorubicin, epirubicin and idarubicin, mitomycin-C, bleomycin, dactinomycin, plicamycin, streptozocin); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); angiogenesis inhibitors (e.g. tasquinimod), tubuline inhibitors; DNA synthesis inhibitors, PARP inhibitors, topoisomerase inhibitors (e.g. epipodophyllotoxins such as for example etoposide and etopophos, teniposide, amsacrin, topotecan, irinotecan, mitoxantrone), serine/threonine kinase inhibitors (e.g. PDK 1 inhibitors, Raf inhibitors, A-Raf inhibitors, B-Raf inhibitors, CRaf inhibitors, mTOR inhibitors, mTORC1/2 inhibitors, PI3K inhibitors, PI3Ka inhibitors, dual mTOR/PI3K inhibitors, STK 33 inhibitors, AKT inhibitors, PLK 1 inhibitors, inhibitors of CDKs, Aurora kinase inhibitors), tyrosine kinase inhibitors (e.g. PTK2/FAK inhibitors), protein protein interaction inhibitors (e.g. IAP activator, Mcl-1, MDM2/MDMX), MEK inhibitors, ERK inhibitors, FLT3 inhibitors, BRD4 inhibitors, IGF-1 R inhibitors, TRAILR2 agonists, Bcl-xL inhibitors, Bcl-2 inhibitors, Bcl-2/Bcl-xL inhibitors, ErbB receptor inhibitors, BCR-ABL inhibitors, ABL inhibitors, Src inhibitors, SHP2 inhibitors, rapamycin analogs (e.g. everolimus, temsirolimus, ridaforolimus, sirolimus), androgen synthesis inhibitors, androgen receptor inhibitors, DNMT inhibitors, HDAC inhibitors, ANGI/2 inhibitors, CYP17 inhibitors, radiopharmaceuticals, proteasome inhibitors, immunotherapeutic agents such as immune checkpoint inhibitors (e.g. CTLA4, PD1, PD-L1, PD-L2, LAG3, and TIM3 binding molecules/immunoglobulins, such as e.g. ipilimumab, nivolumab, pembrolizumab), ADCC (antibody-dependent cell-mediated cytotoxicity) enhancers (e.g. anti-CD33 antibodies, anti-CD37 antibodies, anti-CD20 antibodies), t-cell engagers (e.g. bi-specific T-cell engagers (BiTEs@) like e.g. CD3×BCMA, CD3×CD33, CD3×CD19), PSMA×CD3), tumor vaccines and various chemotherapeutic agents such as amifostin, anagrelid, clodronat, filgrastin, interferon, interferon alpha, leucovorin, procarbazine, levamisole, mesna, mitotane, pamidronate and porfimer.

In certain embodiments, a compound of the present disclosure may be combined with one or more additional therapeutic agent. In other embodiments, a compound of the present invention may be combined with two or more additional therapeutic agents. In one aspect, the additional therapeutic agents are selected from selective estrogen receptor degraders, Protac-mediated estrogen receptor inhibitors, complete estrogen receptor antagonists, sarcoplasmic reticulum calcium ATPase inhibitors, CDK2 inhibitors, CDK2/4/6 inhibitors, CDK4/6 inhibitors, aromatase inhibitors, KRAS inhibitors, RAF, MEK, or ERK inhibitors, AKT inhibitors, mTOR inhibitors, tyrosine kinase inhibitors, DNA Synthesis inhibitors, SHP2 inhibitors, BCL-2 family inhibitors, immune checkpoint inhibitors, and SRC inhibitors.

In another aspect, the additional therapeutic agents are selected from palbociclib, abemaciclib, ribociclib, letrozole, fulvestrant, palazestrant, camizestrant, elacestrant, imlunestrant exernestane, anastrozole, LSZ102, cetuximab, trastuzumab, pertuzumab, nab-paclitaxel, tucatinib, vinorelbine, evexomostat, eribulin, capecitabine, gedatolisib, tamoxifen, zotatifin, neratinib, giredestrant, talazoparib, pembroluzimab, metformin, AMG-510, trametinib, dabrafenib, LY 3214996, PF-07104091, everolimus, and capivasertib. In one asepct, each agent is provided in a separate dosage form. In one aspect, one or more agent is provided in a combined dosage form.

Also provided herein is a compound of the present disclosure, or a salt thereof, or a pharmaceutical composition thereof as defined herein for use in therapy.

Also provided herein is a compound of the present disclosure, or a pharmaceutically acceptable salt or solvate thereof or a pharmaceutical composition thereof as defined herein for use in the treatment of cancer.

Also provided herein is a compound of the present disclosure, or a pharmaceutically acceptable salt or solvate thereof for use in the inhibition of PI3K or a mutant variant thereof.

In some embodiments the mutant varient thereof may be a PI3Kα mutation of H1047, E545, E542, N345, E726, C420, Q546, G118, E453, Q546, G1049, M1043, K111, E81, N1044, and E110. In other embodiments, the mutant varient thereof may be a PI3Kα mutation of H1047, E545, E542, N345, E726, C420, Q546, G118, E453, Q546, G1049, M1043, K111, E81, N1044, E110, R88, 1391, R108H, Y1021, R93W, T1025A, R93, V344, R38, P539, E418, and E970

Also provided herein is a compound of the present disclosure, or a pharmaceutically acceptable salt or solvate thereof or a pharmaceutical composition thereof as defined herein, for use in the treatment of a PI3K-associated, or mutant variant, disease or disorder.

Also provided herein is the use of a compound of the present disclosure, or a pharmaceutically acceptable salt or solvate thereof, as defined herein in the manufacture of a medicament for the treatment of cancer.

Also provided herein is a use of a compound of the present disclosure or a pharmaceutically acceptable salt or solvate thereof, as defined herein in the manufacture of a medicament for the inhibition of activity of PI3K or a mutant variant.

Also provided herein is the use of a compound of the present disclosure, or a pharmaceutically acceptable salt or solvate thereof, as defined herein, in the manufacture of a medicament for the treatment of a PI3K-associated disease or disorder.

Also provided herein is a method for treating cancer in a patient in need thereof, the method comprising (a) determining that cancer is associated with a PI3K mutation (e.g., as determined using an approved assay or kit); and (b) administering to the patient a therapeutically effective amount of a compound of the present disclosure, or a pharmaceutically acceptable salt thereof, or a pharmaceutical composition thereof.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

The present disclosure explicitly encompasses those compounds presented below, including salt forms thereof. The present disclosure also encompasses those compounds presented below, including stereoisomers thereof. A composition comprising a therapeutically acceptable amount of any of these compounds is also within the scope of the disclosure. The composition may further comprise a pharmaceutically acceptable excipient, diluent, carrier, or mixture thereof. Such a composition may be administered to a subject in need thereof to treat or control a disease or disorder mediated, in whole or in part, directly or indirectly, by PI3K or a mutant form thereof. The composition may further comprise an additional active agent, as described herein.

### SYNTHETIC EXAMPLES

The following examples provide a more detailed description of the process conditions for preparing compounds of the present disclosure. It is to be understood, however, that the invention, as fully described herein and as recited in the claims, is not intended to be limited by the details of the following schemes or modes of preparation.

Certain abbreviations may be used in describing the examples of the present disclosure. The abbreviations are believed to be used consistently within commonly accepted use of those skilled in the art.

The compounds of the present disclosure may be prepared from commercially available reagents using the synthetic methods and reaction schemes described herein, or using other reagents and conventional methods well known to those skilled in the art.

As will be apparent, certain compounds of the present disclosure may not only represent a final product having the desired biological effect, but also capable of serving as a synthetic intermediate to yet an alternative final product compound of the present disclosure.

For compounds of the present disclosure that contain one or more chiral center, examples may be presented as racemic mixtures or may be characterized as having a particular stereochemical orientation. Stereochemical configuration herein has been labeled based on information from prior art regarding a preferential biology of one enantiomer over the other. Absolute configuration has not been characterized. All steriosomers for the depicted compounds are aspects of the invention. Moreover, although the following examples recite characterizing data for particular isomers, the order is random and does not necessarily provide any indication regarding the order of the recited compound names or depictions.

The following examples provide a description of the process conditions for preparing compounds on the present invention. It is to be understood, however, that the invention, as fully described herein and as recited in the claims, is not intended to be limited by the details of the following schemes or modes of preparation.

Certain abbreviations may be used in describing the examples of the present disclosure. The abbreviations are believed to be used consistently within commonly accepted use of those skilled in the art.

In the following schemes, general substituent groups are represented with assignments that may not align with the formulae of the present disclosure. The following schemes provide a key for such substituent groups that should be followed for the schemes and not applied to the formulae of the present disclosure.

Compounds of the present disclosure may be made following the synthetic descriptions herein provided, making appropriate modifications as would be appreciated by those of skill in the art.

Synthetic support for referenced portions of one or more embodiments of the present disclosure is hereby made to the synthetic teaching and examples disclosed of one or more of the following patent publications: WO 2024/026423, WO 2024/008122, WO 2024/000401, WO 2023/239710, WO 2023/230262, WO 2023/207881, WO 2023/205680, WO 2023/192416, WO 2023/159155, WO 2023/081209, WO 2023/078401, WO 2023/060262, WO 2024/026419, WO 2024/026424, and WO 2021/202964.

Compounds of the present disclosure may be synthesized following the teachings of the schemes and specification.

### EXAMPLES

### Experimental Procedures:

### Example 1

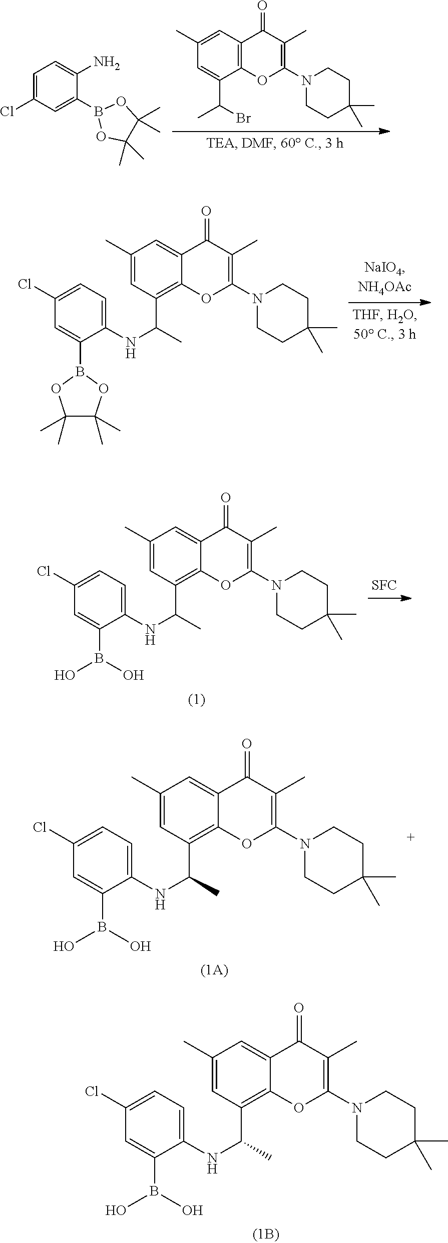

### Preparation of 8-[1-[4-chloro-2-(4,4,5,5-tetramethyl-1,3,2-dioxaborolan-2-yl)anilino]ethyl]-2-(4,4-dimethyl-1-piperidyl)-3,6-dimethyl-chromen-4-one

A mixture of 4-chloro-2-(4,4,5,5-tetramethyl-1,3,2-dioxaborolan-2-yl)aniline (258 mg, 1.02 mmol, 1.6 eq), 8-(1-bromoethyl)-2-(4,4-dimethyl-1-piperidyl)-3,6-dimethyl-chromen-4-one (250 mg, 637.22 μmol, 1 eq) and TEA (129 mg, 1.27 mmol, 177.39 μL, 2 eq) in DMF (2 mL) was degassed and purged with N₂ for 3 times at 20° C., and then the mixture was stirred at 60° C. for 3 h under N₂ atmosphere. After cooling to room temperature, the reaction mixture was quenched by adding water (5 mL) at 0° C. and solid formed. The reaction mixture was filtrated and the filter cake was collected to give a crude product.

The crude product was purified by column chromatography (SiO₂, Petroleum ether/Ethyl acetate=5/1 to 2/1) to give the title compound (228 mg, 403.58 μmol, 63.33% yield) as a brown solid. ¹H NMR (CDCl₃, 400 MHz) δ 7.88 (s, 1H), 7.59 (d, J=2.4 Hz, 1H), 7.40 (s, 1H), 7.06 (dd, J=2.0, 8.4 Hz, 1H), 6.35 (s, 1H), 6.18 (d, J=8.8 Hz, 1H), 4.98-4.94 (m, 1H), 3.45-3.35 (s, 4H), 2.37 (s, 3H), 2.08 (s, 3H), 1.60 (d, J=6.4 Hz, 3H), 1.55-1.49 (m, 4H), 1.38 (s, 12H), 1.04 (s, 6H).

### Preparation of [5-chloro-2-[1-[2-(4,4-dimethyl-1-piperidyl)-3,6-dimethyl-4-oxo-chromen-8-yl]ethylamino]phenyl]boronic acid

To a solution of 8-[1-[4-chloro-2-(4,4,5,5-tetramethyl-1,3,2-dioxaborolan-2-yl)anilino]ethyl]-2-(4,4-dimethyl-1-piperidyl)-3,6-dimethyl-chromen-4-one (180 mg, 318.61 μmol, 1 eq) in THF (1.5 mL)/H₂O (0.5 mL) was added NH₄OAc (73 mg, 955.84 μmol, 3 eq) and NaIO₄ (204 mg, 955.84 μmol, 52.97 μL, 3 eq) in turns at 20° C., the reaction mixture was stirred at 50° C. for 3 h. After cooling to room temperature, the reaction mixture was poured into ice-water (5 mL), stirred at 20° C. for 0.5 h, and then extracted with EtOAc (5 mL×3). The combined organic phase was washed with brine (5 mL×2), dried over Na₂SO₄, filtered and concentrated under reduced pressure to give a residue. The residue was purified by prep-HPLC (column: Waters Xbridge Prep OBD C18 150×40 mm×10 um; mobile phase: [H₂O (10 mM NH₄HCO₃)-AC□]; gradient: 50%-80% B over 8.0 min) to give the title compound (35 mg, 71.85 μmol, 22.55% yield) as an off-white solid. ¹H NMR (DMSO-d₆, 400 MHz) δ 8.52 (s, 2H), 7.62 (s, 1H), 7.57 (d, J=2.8 Hz, 1H), 7.36 (d, J=2.0 Hz, 1H), 7.10 (d, J=6.0 Hz, 1H), 7.04 (dd, J=2.8, 8.8 Hz, 1H), 6.20 (d, J=8.8 Hz, 1H), 4.97-4.89 (m, 1H), 3.42-3.37 (m, 4H), 2.30 (s, 3H), 1.91 (s, 3H), 1.54-1.43 (m, 7H), 0.99 (s, 6H). MS (ESI): mass calcd. For C₂₆H₃₂BClN₂O₄ 482.21, m/z found 483.3 [M+H]⁺. HPLC: 97.98% (220 nm), 97.79% (254 nm).

### Preparation of [5-chloro-2-[[(1S)-1-[2-(4,4-dimethyl-1-piperidyl)-3,6-dimethyl-4-oxo-chromen-8-yl]ethyl]amino]phenyl]boronic acid and [5-chloro-2-[[(R)-1-[2-(4,4-dimethyl-1-piperidyl)-3,6-dimethyl-4-oxo-chromen-8-yl]ethyl]amino]phenyl]boronic acid

[5-chloro-2-[1-[2-(4,4-dimethyl-1-piperidyl)-3,6-dimethyl-4-oxo-chromen-8-yl]ethylamino]phenyl]boronic acid (30.0 mg, 62.14 μmol, 1 eq) was separated by SFC (column: REGIS (s, s) WHELK-O 1 (250 mm×30 mm×5 um); mobile phase: [CO₂-EtOH]; B %: 50%, isocratic elution mode) to give the title compounds, Isomer 1 (7.9 mg, 16.36 μmol, 26.33% yield) as an off-white solid and the title compound, Isomer 2 (11.1 mg, 22.99 μmol, 37.00% yield) as an off-white solid. Isomer 1: ¹H NMR (DMSO-d₆, 400 MHz) δ 8.51 (s, 2H), 7.62 (s, 1H), 7.57 (d, J=2.4 Hz, 1H), 7.36 (s, 1H), 7.12-7.08 (m, 1H), 7.06-7.02 (m, 1H), 6.20 (d, J=8.8 Hz, 1H), 4.98-4.89 (m, 1H), 3.42-3.37 (m, 4H), 2.30 (s, 3H), 1.91 (s, 3H), 1.54-1.44 (m, 7H), 0.99 (s, 6H). MS (ESI): mass calcd. For C₂₆H₃₂BClN₂O₄ 482.21, m/z found 483.3 [M+H]⁺. HPLC: 98.62% (220 nm), 98.17% (254 nm). Chiral purity: 100% ee. Isomer 2: ¹H NMR (DMSO-d₆, 400 MHz) δ 8.51 (s, 2H), 7.62 (s, 1H), 7.57 (d, J=2.4 Hz, 1H), 7.36 (s, 1H), 7.12-7.08 (m, 1H), 7.06-7.02 (m, 1H), 6.20 (d, J=8.8 Hz, 1H), 4.97-4.90 (m, 1H), 3.42-3.37 (m, 4H), 2.30 (s, 3H), 1.92 (s, 3H), 1.54-1.44 (m, 7H), 0.99 (s, 6H). MS (ESI): mass calcd. For C₂₆H₃₂BClN₂O₄ 482.21, m/z found 483.3 [M+H]⁺. HPLC: 97.94% (220 nm), 96.63% (254 nm). Chiral purity: 90.68% ee.

### Example 2

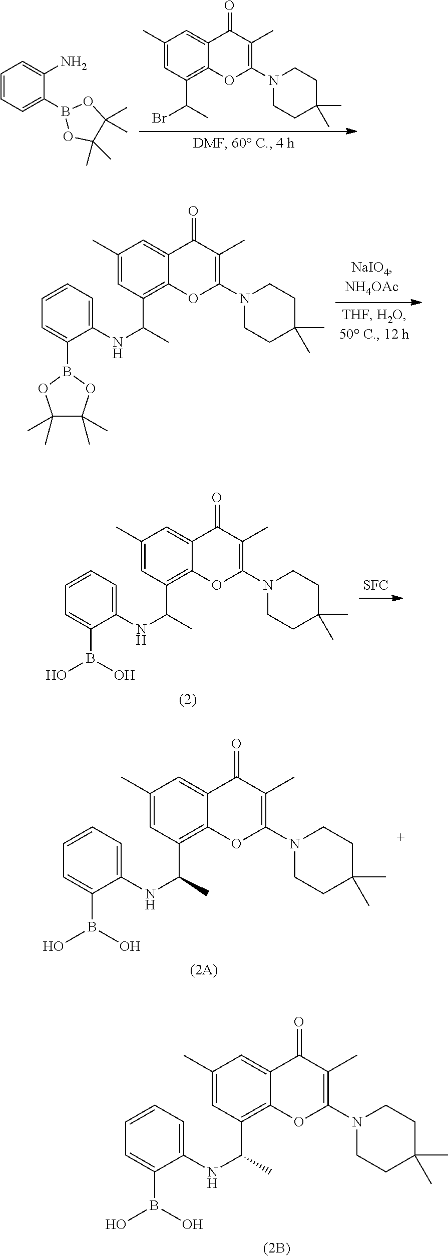

### Preparation of 2-(4,4-dimethyl-1-piperidyl)-3,6-dimethyl-8-[1-[2-(4,4,5,5-tetramethyl-1,3,2-dioxaborolan-2-yl)anilino]ethyl]chromen-4-one

A solution of 8-(1-bromoethyl)-2-(4,4-dimethyl-1-piperidyl)-3,6-dimethyl-chromen-4-one (250 mg, 637.22 μmol, 1 eq) and 2-(4,4,5,5-tetramethyl-1,3,2-dioxaborolan-2-yl)aniline (279.21 mg, 1.27 mmol, 2 eq) in DMF (2 mL) was stirred at 60° C. for 4 h. The reaction mixture was quenched with water (20 mL) and then extracted with MTBE (10 mL, ×3). The combined organic phases were washed with brine (10 mL×2), dried over Na₂SO₄, filtered and concentrated under reduced pressure to give the title compound (300 mg, crude) as a yellow solid, which was used for next step without further purification.

### Preparation of [2-[1-[2-(4,4-dimethyl-1-piperidyl)-3,6-dimethyl-4-oxo-chromen-8-yl]ethylamino]phenyl]boronic acid

To a solution of 2-(4,4-dimethyl-1-piperidyl)-3,6-dimethyl-8-[1-[2-(4,4,5,5-tetramethyl-1,3,2-dioxaborolan-2-yl)anilino]ethyl]chromen-4-one (300 mg, 339.30 μmol, 1 eq) in THF (2 mL)/H₂O (1 mL) was added NH₄OAc (79 mg, 1.02 mmol, 3 eq) and NaIO₄ (218 mg, 1.02 mmol, 56.40 μL, 3 eq) in turns at 20° C., the reaction mixture was stirred at 50° C. for 12 h. After the reaction mixture was cooled to room temperature, the reaction mixture was poured into ice-water (20 mL) and extracted with EtOAc (10 mL×3). The combined organic phase was washed with brine (10 mL), dried over Na₂SO₄, filtered and concentrated under reduced pressure. The crude product was purified by reversed-phase HPLC (column: Waters Xbridge Prep OBD C18 150×40 mm×10 um; mobile phase: [water (NH4HCO3)-AC□]; gradient: 35%-65% B over 8 min) to give the title compound (39 mg, 86.54 μmol, 25.50% yield) as an off white solid. ¹H NMR (DMSO-d₆, 400 MHz) δ 8.28 (s, 2H), 7.61-7.57 (m, 2H), 7.40 (s, 1H), 7.03-7.00 (m, 2H), 6.46 (t, J=6.8 Hz, 1H), 6.20 (d, J=8.4 Hz, 1H), 4.97-4.94 (m, 1H), 3.41-3.40 (m, 4H), 2.30 (s, 3H), 1.92 (s, 3H), 1.53 (d, J=6.4 Hz, 3H), 1.47-1.46 (m, 4H), 0.99 (s, 6H). MS (ESI): mass calcd. For C₂₆H₃₃BN₂O₄ 448.25, m/z found 449.2 [M+H]⁺. HPLC: 94.69% (220 nm), 96.45% (254 nm).

### Preparation of [2-[[(1S)-1-[2-(4,4-dimethyl-1-piperidyl)-3,6-dimethyl-4-oxo-chromen-8-yl]ethyl]amino]phenyl]boronic acid and [2-[[(R)-1-[2-(4,4-dimethyl-1-piperidyl)-3,6-dimethyl-4-oxo-chromen-8-yl]ethyl]amino]phenyl]boronic acid

[2-[1-[2-(4,4-dimethyl-1-piperidyl)-3,6-dimethyl-4-oxo-chromen-8-yl]ethylamino]phenyl]boronic acid (36 mg, 80.29 μmol, 1 eq) was separated by SFC separation (column: REGIS (s,s) WHELK-O 1 (250 mm×30 mm×5 um); mobile phase: [CO₂-EtOH]; B %:50%, isocratic elution mode) to give the title compound, Isomer 1 (7.5 mg, 16.28 μmol, 20.28% yield) as a white solid and the title compound, Isomer 2 (10.1 mg, 22.32 μmol, 27.80% yield) as a white solid. Isomer 1: ¹H NMR (DMSO-d₆, 400 MHz) δ 8.33 (s, 2H), 7.63 (s, 1H), 7.60 (d, J=6.8 Hz, 1H), 7.41 (s, 1H), 7.06 (d, J=6.4 Hz, 1H), 7.03 (d, J=7.2 Hz, 1H), 6.48 (t, J=7.6 Hz, 1H), 6.20 (d, J=8.0 Hz, 1H), 5.00-4.94 (m, 1H), 3.44-3.42 (m, 4H), 2.32 (s, 3H), 1.93 (s, 3H), 1.53 (d, J=6.4 Hz, 3H), 1.48-1.47 (m, 4H), 1.00 (s, 6H). MS (ESI): mass calcd. For C₂₆H₃₃BN₂O₄ 448.25, m/z found 449.2 [M+H]⁺. HPLC: 99.10% (220 nm), 98.71% (254 nm).

Chiral purity: 100% ee. Isomer 2: ¹H NMR (DMSO-d₆, 400 MHz) δ 8.32 (s, 2H), 7.61 (s, 1H), 7.59 (d, J=7.2 Hz, 1H), 7.43 (s, 1H), 7.06 (d, J=6.4 Hz, 1H), 7.01 (d, J=7.6 Hz, 1H), 6.48 (t, J=6.8 Hz, 1H), 6.20 (d, J=8.0 Hz, 1H), 4.97-4.92 (m, 1H), 3.42-3.40 (m, 4H), 2.30 (s, 3H), 1.92 (s, 3H), 1.53 (d, J=6.4 Hz, 3H), 1.47-1.46 (m, 4H), 0.99 (s, 6H). MS (ESI): mass calcd. For C₂₆H₃₃BN₂O₄ 448.25, m/z found 449.2 [M+H]⁺. HPLC: 97.33% (220 nm), 97.32% (254 nm). Chiral purity: 93.60% ee.

### Example 3

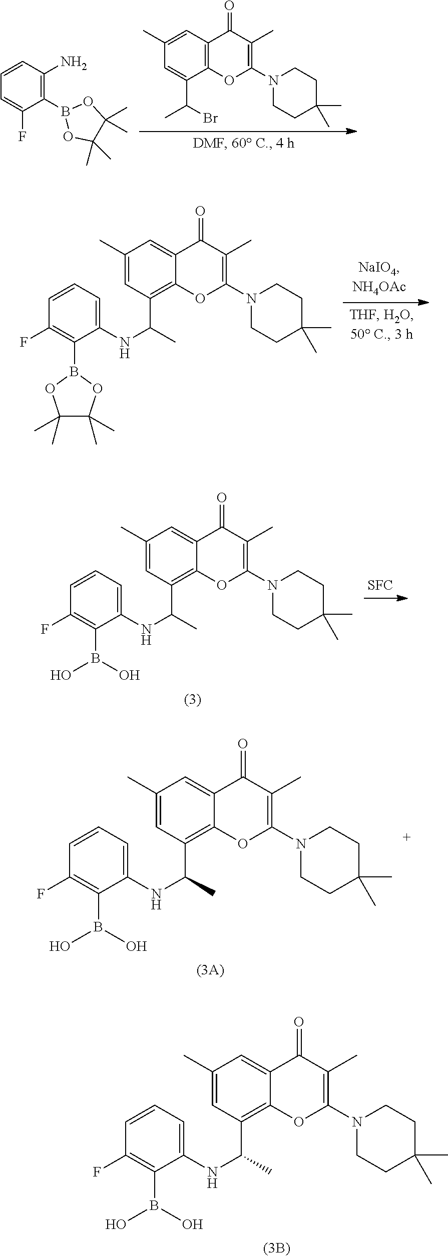

### Preparation of 2-(4,4-dimethylpiperidin-1-yl)-8-(1-((3-fluoro-2-(4,4,5,5-tetramethyl-1,3,2-dioxaborolan-2-yl)phenyl)amino)ethyl)-3,6-dimethyl-4H-chromen-4-one

To a solution of 8-(1-bromoethyl)-2-(4,4-dimethyl-1-piperidyl)-3,6-dimethyl-chromen-4-one (250 mg, 637.22 μmol, 1 eq) in DMF (3 mL) was added 3-fluoro-2-(4,4,5,5-tetramethyl-1,3,2-dioxaborolan-2-yl)aniline (250 mg, 1.05 mmol, 1.65 eq) in one portion at 25° C., the reaction mixture was stirred at 60° C. for 4 h. The reaction mixture was quenched by adding water (10 mL) at 0° C. and the resulting suspension was directly filtered. The filter cake was further triturated with water at 0° C. for 10 min to give the title compound (300 mg, crude) as a white solid, which was used directly for next step without further purification.

### Preparation of (2-((1-(2-(4,4-dimethylpiperidin-1-yl)-3,6-dimethyl-4-oxo-4H-chromen-8-yl)ethyl)amino)-6-fluorophenyl)boronic acid

To a solution of 2-(4,4-dimethyl-1-piperidyl)-8-[1-[3-fluoro-2-(4,4,5,5-tetramethyl-1,3,2-dioxaborolan-2-yl)anilino]ethyl]-3,6-dimethyl-chromen-4-one (250 mg, 455.79 μmol, 1 eq) in THF (3 mL)/H₂O (1 mL) added NaIO₄ (293 mg, 1.37 mmol, 75.77 μL, 3 eq) and NH₄OAc (106 mg, 1.37 mmol, 3 eq) in one portion at 25° C., the reaction mixture was stirred at 50° C. for 3 h. The reaction mixture was diluted with H₂O (10 mL) and extracted with EtOAc (10 mL×3). The combined organic layers were washed with brine (10 mL), dried over with Na₂SO₄, filtered and concentrated under reduced pressure to give a residue. The residue was purified by prep-HPLC (neutral condition; column: Waters Xbridge Prep OBD C18 150×40 mm×10 um; mobile phase: [water (NH₄HCO₃)-ACN]; B %: 55%-75%, 8 min) to give the title compound (37 mg, 79.34 μmol, 17.41% yield) as a white solid. ¹H NMR (DMSO-d₆, 400 MHz) 8.44 (s, 2H), 7.62-7.61 (d, J=1.6 Hz, 1H), 7.42-7.41 (d, J=2.0 Hz, 1H), 6.09-6.99 (d, J=7.2 Hz, 1H), 6.41-6.40 (d, J=6.0 Hz, 1H), 6.26-6.22 (t, J=8.8 Hz, 1H), 6.05-6.03 (d, J=8.4 Hz, 1H), 4.96-4.93 (m, 1H), 3.41-3.40 (d, J=2.8 Hz, 4H), 2.31 (s, 3H), 1.91 (s, 3H), 1.52-1.51 (d, J=6.8 Hz, 3H), 1.46-1.45 (d, J=2.8 Hz, 4H), 0.98 (s, 6H). MS (ESI): mass calcd. For C₂₆H₃₂BFN₂O₄ 466.36, m/z found 467.2 [M+H]⁺. HPLC: 97.09% (220 nm), 96.78% (254 nm).

### Preparation of (S)-(2-((1-(2-(4,4-dimethylpiperidin-1-yl)-3,6-dimethyl-4-oxo-4H-chromen-8-yl)ethyl)amino)-6-fluorophenyl)boronic acid and (R)-(2-((1-(2-(4,4-dimethylpiperidin-1-yl)-3,6-dimethyl-4-oxo-4H-chromen-8-yl)ethyl)amino)-6-fluorophenyl)boronic acid

(2-((1-(2-(4,4-dimethylpiperidin-1-yl)-3,6-dimethyl-4-oxo-4H-chromen-8-yl)ethyl)amino)-6-fluorophenyl)boronic acid (30 mg) was further separated by SFC (condition: column: REGIS (s, s) WHELK-O1 (250 mm×30 mm, 5 um); mobile phase: [CO₂-EtOH]; B %: 50%, isocratic elution mode) to give the title compound, Isomer 1 (6.6 mg, 14.59 μmol, 26.67% yield) as a white solid and the title compound, Isomer 2 (7.8 mg, 16.18 μmol, 29.58% yield) as a white solid. Isomer 1: ¹H NMR (DMSO-d₆, 400 MHz) 8.45 (d, J=1.2 Hz 2H), 7.62 (s, 1H), 7.42 (d, J=2.0 Hz, 1H), 7.03-6.98 (m, 1H), 6.42-6.40 (d, J=6.0 Hz, 1H), 6.27-6.22 (t, J=8.8 Hz, 1H), 6.05-6.03 (d, J=8.0 Hz, 1H), 4.98-4.92 (m, 1H), 3.48-3.40 (m, 4H), 2.32-2.31 (d, J=4.0 Hz, 3H), 1.92 (s, 3H), 1.53-1.51 (d, J=6.8 Hz, 3H), 1.48-1.45 (m, 4H), 0.99 (s, 6H). MS (ESI): mass calcd. For C₂₆H₃₂BFN₂O₄ 466.36, m/z found 467.2 [M+H]⁺. HPLC: 98.68% (220 nm), 97.8% (254 nm). Chiral purity: 100% ee. Isomer 2: ¹H NMR (DMSO-d₆, 400 MHz) 8.45 (d, J=1.2 Hz 2H), 7.62 (s, 1H), 7.42 (d, J=2.0 Hz, 1H), 7.03-6.98 (m, 1H), 6.42-6.40 (d, J=6.0 Hz, 1H), 6.27-6.22 (t, J=8.4 Hz, 1H), 6.05-6.03 (d, J=8.0 Hz, 1H), 4.98-4.92 (t, J=6.4 Hz, 1H), 3.42-3.40 (m, 4H), 2.33-2.31 (m, 3H), 1.92-1.91 (d, J=4.0 Hz, 3H), 1.53-1.51 (d, J=6.4 Hz, 3H), 1.48-1.45 (m, 4H), 0.99 (s, 6H). MS (ESI): mass calcd. For C₂₆H₃₂BFN₂O₄ 466.36, m/z found 467.2 [M+H]⁺. HPLC: 95.5% (220 nm), 94.5% (254 nm). Chiral purity: 99.76% ee.

### Example 4

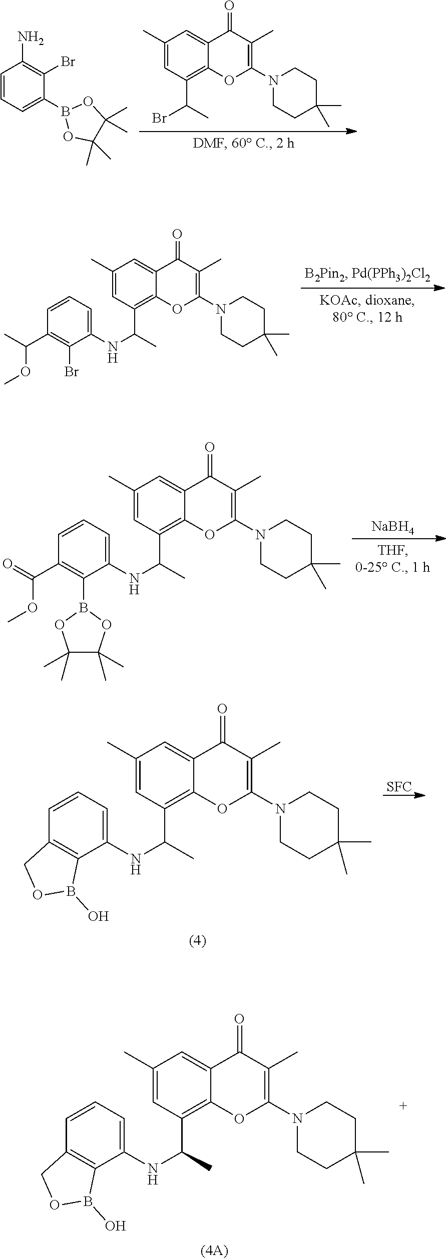

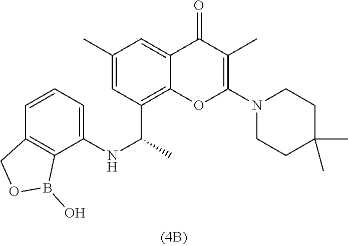 [SMILES: Cc1cc([C@H](C)Nc2cccc3c2B(O)OC3)c2oc(N3CCC(C)(C)CC3)c(C)c(=O)c2c1]

### Preparation of methyl 2-bromo-3-[1-[2-(4,4-dimethyl-1-piperidyl)-3,6-dimethyl-4-oxo-chromen-8-yl]ethylamino]benzoate

A solution of 8-(1-bromoethyl)-2-(4,4-dimethyl-1-piperidyl)-3,6-dimethyl-chromen-4-one (500 mg, 1.3 mmol, 1.0 eq) and methyl 3-amino-2-bromo-benzoate (587 mg, 2.6 mmol, 2.0 eq) in DMF (5 mL) was stirred at 60° C. for 2 h. The reaction mixture was cooled to 25° C., quenched with H₂O (20 mL) and extracted with MTBE (10 mL×3). The combined organic phases were washed with brine (10 mL), dried over Na₂SO₄, filtered and concentrated under reduced pressure. The crude product was purified by flash silica gel chromatography (Biotage®; 20 g SepaFlash® Silica Flash Column, Eluent of 0~25% Petroleum ether/Ethyl acetate gradient@50 mL/min) to give the title compound (400 mg, 738.7 mol, 57.96% yield) as a pale yellow solid. ¹H NMR (DMSO-d₆, 400 MHz) δ 7.63 (s, 1H), 7.45 (s, 1H), 7.15-7.11 (m, 1H), 6.84-6.82 (m, 1H), 6.58-6.56 (m, 1H), 5.62-5.60 (m, 1H), 5.08-5.04 (m, 1H), 3.82 (s, 3H), 3.42-3.39 (m, 4H), 2.31 (s, 3H), 1.91 (s, 3H), 1.64-1.63 (m, 3H), 1.47-1.44 (m, 4H), 0.98 (s, 6H).

### Preparation of methyl 3-((1-(2-(4,4-dimethylpiperidin-1-yl)-3,6-dimethyl-4-oxo-4H-chromen-8-yl)ethyl)amino)-2-(4,4,5,5-tetramethyl-1,3,2-dioxaborolan-2-yl)benzoate

A mixture of methyl 2-bromo-3-[1-[2-(4,4-dimethyl-1-piperidyl)-3,6-dimethyl-4-oxo-chromen-8-yl]ethylamino]benzoate (75 mg, 138.51 μmol, 1 eq), B₂Pin₂ (176 mg, 692.55 μmol, 5 eq), KOAc (41 mg, 415.53 μmol, 3 eq) and Pd(PPh₃)₂Cl₂ (10 mg, 13.85 μmol, 0.1 eq) in dioxane (2 mL) was degassed and purged with N₂ for 3 times at 25° C., and then the mixture was stirred at 80° C. for 12 h under N₂ atmosphere. 2 parallel reactions were combined for work up. The reaction mixture was filtered and the filtrate was concentrated under reduced pressure to give a residue. The residue was purified by flash silica gel chromatography (Biotage®; 12 g SepaFlash® Silica Flash Column, Eluent of 0~35% Ethyl acetate/Petroleum ether gradient@45 mL/min) to give the title compound (280 mg, crude) as light yellow oil. ¹H NMR (CDCl₃, 400 MHz) δ 7.86 (s, 1H), 7.41 (s, 1H), 7.15 (d, J=7.2 Hz, 1H), 7.09 (d, J=8.0 Hz, 1H), 6.37 (d, J=8.0 Hz, 1H), 5.15-5.11 (m, 1H), 5.02-4.97 (m, 1H), 3.88 (s, 3H), 3.41-3.78 (m, 4H), 2.35 (s, 3H), 2.07 (s, 3H), 1.59 (d, J=6.4 Hz, 3H), 1.51-1.49 (m, 4H), 1.43 (s, 12H), 1.02 (s, 6H).

### Preparation of 2-(4,4-dimethyl-1-piperidyl)-8-[1-[(1-hydroxy-3H-2,1-benzoxaborol-7-yl)amino]ethyl]-3,6-dimethyl-chromen-4-one

To a solution of methyl 3-[1-[2-(4,4-dimethyl-1-piperidyl)-3,6-dimethyl-4-oxo-chromen-8-yl]ethylamino]-2-(4,4,5,5-tetramethyl-1,3,2-dioxaborolan-2-yl)benzoate (180 mg, 305.84 μmol, 1 eq) in THF (2 ml) was added NaBH₄ (58 mg, 1.53 mmol, 5 eq) at 0° C. in portions. The mixture was warmed to 25° C. and stirred at 25° C. for 1 h. The reaction mixture was cooled to 0° C. and quenched by adding H2O (8 mL) at 0° C. Then the mixture was adjusted to pH=7 with HCl (2 N) and extracted with EtOAc (10 mL×3). The combined organic layers were dried over Na₂SO₄, filtered and concentrated under reduced pressure to give a residue. The residue was purified by prep-HPLC (column: Waters Xbridge BEH C18 100×30 mm×10 um; mobile phase: [H₂O (10 mM NH₄HCO₃)-AC□]; gradient: 50%-80% B over 8.0 min) to give the title compound (50 mg, 108.61 μmol, 35.51% yield) as a white solid. ¹H NMR (DMSO-d₆, 400 MHz) δ 9.14 (s, 1H), 7.62 (s, 1H), 7.45 (s, 1H), 7.09 (t, J=7.6 Hz, 1H), 6.53 (d, J=7.2 Hz, 1H), 6.15 (d, J=8.0 Hz, 1H), 5.53 (d, J=6.8 Hz, 1H), 5.06 (t, J=6.8 Hz, 1H), 4.85 (s, 2H), 3.43-3.40 (m, 4H), 2.32 (s, 3H), 1.92 (s, 3H), 1.57 (d, J=6.8 Hz, 3H), 1.47-1.45 (m, 4H), 0.99 (s, 6H). MS (ESI): mass calcd. For C₂₁H₃₃BN₂O₄ 460.38, m/z found 461.4 [M+H]⁺. HPLC: 94.80% (220 nm), 95.94% (254 nm).

### Preparation of 2-(4,4-dimethyl-1-piperidyl)-8-[(1S)-1-[(1-hydroxy-3H-2,1-benzoxaborol-7-yl)amino]ethyl]-3,6-dimethyl-chromen-4-one and 2-(4,4-dimethyl-1-piperidyl)-8-[(1R)-1-[(1-hydroxy-3H-2,1-benzoxaborol-7-yl)amino]ethyl]-3,6-dimethyl-chromen-4-one

2-(4,4-dimethyl-1-piperidyl)-8-[(1-[1-hydroxy-3H-2,1-benzoxaborol-7-yl)amino]ethyl]-3,6-dimethyl-chromen-4-one (50 mg, 108.61 μmol, 1 eq) was separated by SFC separation (column: ChiralPak IH, 250×30 mm×10 um; mobile phase: [CO₂-MeOH]; B %: 23%, isocratic elution mode) to give the title compound, Isomer 1 (18.3 mg, 39.6 μmol, 36.4% yield) as a white solid and the title compound, Isomer 2 (17.3 mg, 37.5 μmol, 34.5% yield) as a white solid. Isomer 1: ¹H NMR (DMSO-d₆, 400 MHz) δ 9.16 (s, 1H), 7.62 (s, 1H), 7.45 (s, 1H), 7.09 (t, J=7.6 Hz, 1H), 6.54 (d, J=7.2 Hz, 1H), 6.16 (t, J=7.6 Hz, 1H), 5.55 (d, J=7.2 Hz, 1H), 5.07-5.04 (m, 1H), 4.85 (s, 2H), 3.42-3.40 (m, 4H), 2.31 (s, 3H), 1.91 (s, 3H), 1.58 (d, J=6.8 Hz, 3H), 1.47-1.46 (m, 4H), 0.99 (s, 6H). MS (ESI): mass calcd. For C₂₇H₃₃BN₂O₄ 460.25, m/z found 461.3 [M+H]⁺. HPLC: 99.60% (220 nm), 100.00% (254 nm). Chiral purity: 99.92% ee. Isomer 2: ¹H NMR (DMSO-d₆, 400 MHz) δ 9.16 (s, 1H), 7.62 (s, 1H), 7.45 (s, 1H), 7.09 (t, J=7.6 Hz, 1H), 6.54 (d, J=7.2 Hz, 1H), 6.16 (t, J=8.0 Hz, 1H), 5.55 (d, J=6.8 Hz, 1H), 5.07-5.04 (m, 1H), 4.85 (s, 2H), 3.43-3.40 (m, 4H), 2.31 (s, 3H), 1.91 (s, 3H), 1.58 (d, J=6.8 Hz, 3H), 1.47-1.46 (m, 4H), 0.99 (s, 6H). MS (ESI): mass calcd. For C₂₇H₃₃BN₂O₄ 460.25, m/z found 461.3 [M+H]⁺.

HPLC: 99.75% (220 nm), 100.00% (254 nm). Chiral purity: 99.22% ee.

### Example 5

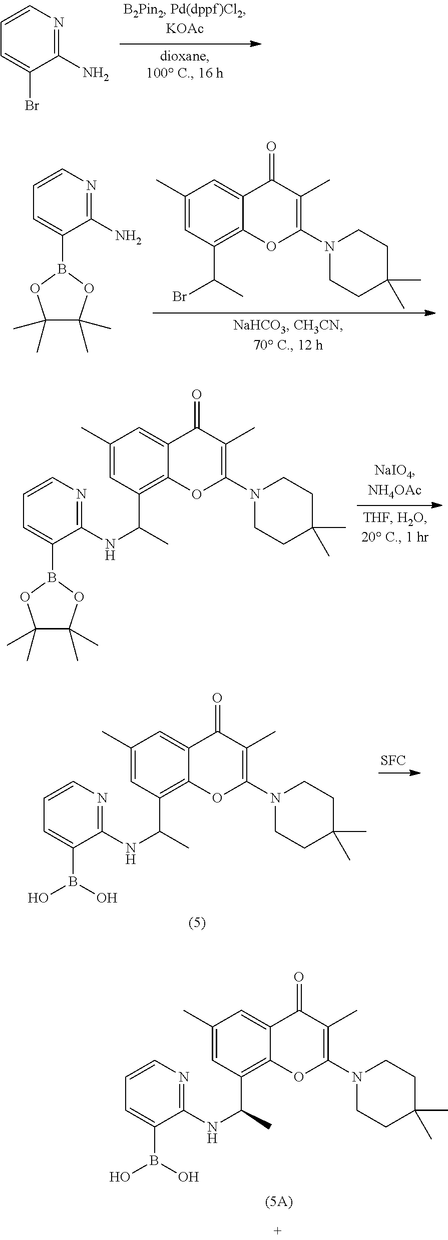

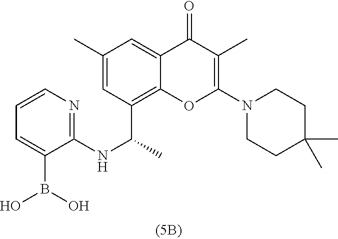 [SMILES: Cc1cc([C@H](C)Nc2ncccc2B(O)O)c2oc(N3CCC(C)(C)CC3)c(C)c(=O)c2c1]

### Preparation of 3-(4,4,5,5-tetramethyl-1,3,2-dioxaborolan-2-yl)pyridin-2-amine

A mixture of 3-bromopyridin-2-amine (1.00 g, 5.78 mmol, 1 eq), 4,4,5,5-tetramethyl-2-(4,4,5,5-tetramethyl-1,3,2-dioxaborolan-2-yl)-1,3,2-dioxaborolane (2.94 g, 11.56 mmol, 2 eq), Pd(dppf)Cl₂ (211.46 mg, 289.00 μmol, 0.05 eq) and KOAc (1.99 g, 20.23 mmol, 3.5 eq) in dioxane (10 mL) was degassed and purged with N₂ for 3 times, and then the reaction mixture was heated to 100° C. and stirred at 100° C. for 16 h under N₂ atmosphere. The reaction mixture was cooled to 20° C. and then filtered through a pad of Celite. The filter cake was washed with DCM (5 mL×2) and the filtrate was concentrated under reduced pressure to give a residue. The crude product was triturated with MTBE/Petroleum ether (20 mL, v/v=1:5) at 20 C for 10 min and the resulting precipitate was collected by filtration to give the title compound (750 mg, 3.41 mmol, 58.96% yield) as off-white solid. ¹H NMR (DMSO-d₆, 400 MHz) δ 8.04 (dd, J=4.8, 2.4 Hz, 1H), 7.67 (dd, J=7.2, 2.4 Hz, 1H), 6.51 (dd, J=7.2, 4.8 Hz, 1H), 6.06 (s, 1H), 1.29 (s, 12H).

### Preparation of 2-(4,4-dimethyl-1-piperidyl)-3,6-dimethyl-8-[1-[[3-(4,4,5,5-tetramethyl-1,3,2-dioxaborolan-2-yl)-2-pyridyl]amino]ethyl]chromen-4-one

To a solution of 3-(4,4,5,5-tetramethyl-1,3,2-dioxaborolan-2-yl)pyridin-2-amine (494 mg, 2.24 mmol, 2.2 eq) and 8-(1-bromoethyl)-2-(4,4-dimethyl-1-piperidyl)-3,6-dimethyl-chromen-4-one (400 mg, 1.02 mmol, 1 eq) in CH₃CN (5 mL) was added NaHCO₃ (257 mg, 3.06 mmol, 119.01 μL, 3 eq) in one portion, the reaction mixture was heated to 70° C. and stirred at 70° C. for 12 h. The reaction mixture was cooled to 25° C., filtered through a pad of Celite and the filter cake was washed with CH₃CN (10 mL×2). The filtrate was concentrated under reduced pressure to give a residue and the residue was purified by flash silica gel chromatography (Biotage®; 20 g SepaFlash® Silica Flash Column, Eluent of 0~100% Ethyl acetate/Petroleum ether gradient@60 mL/min) to give the title compound (120 mg, 225.78 mol, 22.14% yield) as a white solid. ¹H NMR (DMSO-d₆, 400 MHz) δ 8.06 (dd, J=5.2, 2.0 Hz, 1H), 7.69 (dd, J=7.2, 2.8 Hz, 1H), 7.42 (s, 1H), 6.68 (s, 1H), 6.68 (d, J=6.8 Hz, 1H), 6.52 (dd, J=7.2, 5.2 Hz, 1H), 5.67 (t, J=7.2 Hz, 1H), 3.37 (t, J=6.8 Hz, 4H), 2.35 (s, 3H), 1.91 (s, 3H), 1.56 (d, J=6.8 Hz, 3H), 1.47-1.42 (m, 4H), 1.28 (d, J=13.2 Hz, 12H), 0.97 (s, 6H).

### Preparation of [2-[1-[2-(4,4-dimethyl-1-piperidyl)-3,6-dimethyl-4-oxo-chromen-8-yl]ethylamino]-3-pyridyl]boronic acid

To a solution of 2-(4,4-dimethyl-1-piperidyl)-3,6-dimethyl-8-[1-[[3-(4,4,5,5-tetramethyl-1,3,2-dioxaborolan-2-yl)-2-pyridyl]amino]ethyl]chromen-4-one (100 mg, 188.15 μmol, 1 eq) in THF (2 mL)/H₂O (2 mL) was added NH₄OAc (73 mg, 940.75 μmol, 5 eq) and NaIO₄ (201 mg, 940.75 μmol, 52.13 μL, 5 eq) at 20° C. in one portion, the reaction mixture was stirred at 20° C. for 1 h. The reaction mixture was quenched by addition aq. sat. Na₂SO₃ (3 mL) at 20° C., and then extracted with EtOAc (2 mL×3).

The combined organic layers were washed with brine (3 mL), dried over Na₂SO₄, filtered and concentrated under reduced pressure to give a residue. The residue was purified by prep-HPLC (column: Waters Xbridge Prep OBD C18 150×40 mm×10 um; mobile phase: [H₂O (10 mM NH₄HCO₃)-AC□]; gradient: 35%-65% B over 8.0 min). The eluent was lyophilized to give the title compound (34.8 mg, 75.30 μmol, 40.02% yield) as an off-white solid. ¹H NMR (DMSO-d₆, 400 MHz) δ 8.49 (s, 2H), 7.94 (dd, J=6.8, 2.0 Hz, 1H), 7.86 (dd, J=7.2, 2.0 Hz, 1H), 7.61 (s, 1H), 7.43-7.41 (m, 2H), 6.45 (dd, J=6.8, 4.8 Hz, 1H), 5.64 (q, J=6.8 Hz, 1H), 3.35 (t, J=5.2 Hz, 4H), 2.31 (s, 3H), 1.89 (s, 3H), 1.50 (d, J=6.8 Hz, 3H), 1.42-1.37 (m, 4H), 0.96 (s, 6H). MS (ESI): mass calcd. For C₂₅H₃₂BN₃O₄ 449.25, m/z found 450.1 [M+H]⁺. HPLC: 97.23% (220 nm), 99.19% (254 nm).

### Preparation of [2-[[(1S)-1-[2-(4,4-dimethyl-1-piperidyl)-3,6-dimethyl-4-oxo-chrome n-8-yl]ethyl]amino]-3-pyridyl]boronic acid and [2-[[(R)-1-[2-(4,4-dimethyl-1-piperid yl)-3,6-dimethyl-4-oxo-chromen-8-yl]ethyl]amino]-3-pyridyl]boronic acid

[2-[1-[2-(4,4-dimethyl-1-piperidyl)-3,6-dimethyl-4-oxo-chromen-8-yl]ethylamino]-3-pyridyl]boronic acid (30 mg, 66.76 μmol, 1 eq) was separated by SFC (column: REGIS (s, s) WHELK-O 1 (250 mm×30 mm, 5 um); mobile phase: [CO₂-EtOH]; B %: 50%, isocratic elution mode) to give the title compound, Isomer 1 (6.0 mg, 13.35 μmol, 20.00% yield) as a white solid and the title compound, Isomer 2 (8.1 mg, 18.03 μmol, 27.00% yield) as a white solid. Isomer 1: ¹H NMR (DMSO-d₆, 400 MHz) δ 8.48 (s, 2H), 7.94 (d, J=3.2 Hz, 1H), 7.86 (d, J=5.6 Hz, 1H), 7.61 (s, 1H), 7.42 (s, 2H), 6.48-6.44 (m, 1H), 5.69-5.61 (m, 1H), 3.52-3.47 (m, 4H), 2.33 (s, 3H), 1.91 (s, 3H), 1.54-1.41 (m, 7H), 0.96 (s, 6H). MS (ESI): mass calcd. For C₂₅H₃₂BN₃O₄ 449.25, m/z found 450.3 [M+H]⁺. HPLC: 98.75% (220 nm), 99.23% (254 nm). Isomer 2: ¹H NMR (DMSO-d₆, 400 MHz) δ 8.51 (s, 2H), 7.94 (d, J=3.2 Hz, 1H), 7.86 (d, J=6.0 Hz, 1H), 7.61 (s, 1H), 7.43-7.40 (m, 2H), 6.48-6.44 (m, 1H), 5.68-5.61 (m, 1H), 3.53-3.46 (m, 4H), 2.33 (s, 3H), 1.90 (s, 3H), 1.54-1.41 (m, 7H), 0.96 (s, 6H). MS (ESI): mass calcd. For C₂₅H₃₂BN₃O₄ 449.25, m/z found 450.3 [M+H]⁺. HPLC: 98.52% (220 nm), 99.37% (254 nm).

### Example 6

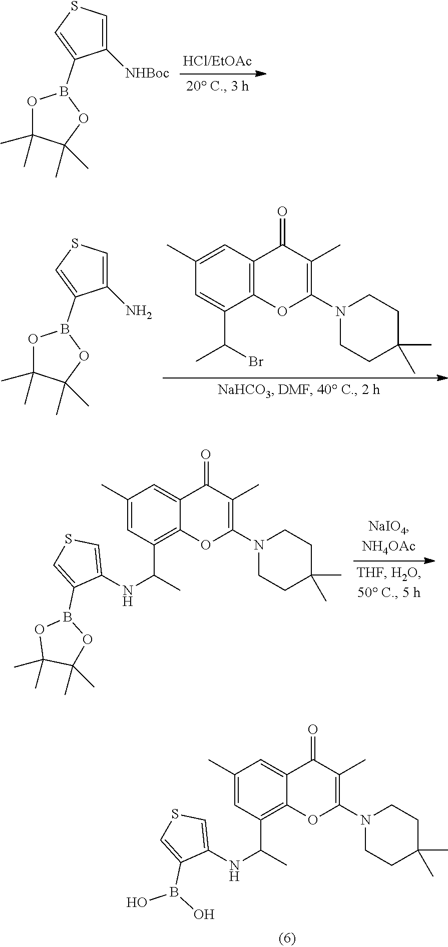

### Preparation of 4-(4,4,5,5-tetramethyl-1,3,2-dioxaborolan-2-yl)thiophen-3-amine

A mixture of tert-butyl N-[4-(4,4,5,5-tetramethyl-1,3,2-dioxaborolan-2-yl)-3-thienyl]carbamate (276 mg, 848 μmol, 1 eq) in HCl/EtOAc (3 mL) was stirred at 20° C. for 3 h. Then the reaction mixture was concentrated under reduced pressure to give the title compound (266 mg, 1.02 mmol, 59.92% yield, HCl) as a white solid. ¹H NMR (CDCl₃, 400 MHz) δ 7.92 (s, 1H), 7.84 (s, 1H), 1.38-1.36 (s, 12H).

### Preparation of 2-(4,4-dimethylpiperidin-1-yl)-3,6-dimethyl-8-(1-((4-(4,4,5,5-tetramethyl-1,3,2-dioxaborolan-2-yl)thiophen-3-yl)amino)ethyl)-4H-chromen-4-one

To a solution of 2-(4,4-dimethylpiperidin-1-yl)-3,6-dimethyl-8-(1-((4-(4,4,5,5-tetramethyl-1,3,2-dioxaborolan-2-yl)thiophen-3-yl)amino)ethyl)-4H-chromen-4-one (266 mg, 1.02 mmol, 1 eq, HCl) in DMF (3 mL) was added 8-(1-bromoethyl)-2-(4,4-dimethyl-1-piperidyl)-3,6-dimethyl-chromen-4-one (362 mg, 923 μmol, 0.9 eq) and NaHCO₃ (152 mg, 1.82 mmol, 70.6 μL, 1.79 eq) in one portion, the reaction mixture was stirred at 40° C. for 2 h. After cooling to room temperature, the reaction mixture was added water (3 mL) and white solid formed. Then the solid was filtered and the filtered cake was dried under reduced pressure to give the title compound (500 mg, crude), which was used directly for next step without further purification.

### Preparation of [4-[1-[2-(4,4-dimethyl-1-piperidyl)-3,6-dimethyl-4-oxo-chromen-8-yl]ethylamino]-3-thienyl]boronic acid

To a solution of 2-(4,4-dimethyl-1-piperidyl)-3,6-dimethyl-8-[1-[[4-(4,4,5,5-tetramethyl-1,3,2-dioxaborolan-2-yl)-3-thienyl]amino]ethyl]chromen-4-one (500 mg, 931 μmol, 1 eq) in THF (4.5 mL)/H₂O (1.5 mL) was added NaIO₄ (598 mg, 2.80 mmol, 155 μL, 3 eq) and NH₄OAc (216 mg, 2.80 mmol, 3 eq) in turns at 20° C., The reaction mixture was stirred at 50° C. for 5 h. After cooling to room temperature, the reaction mixture was diluted with H₂O (10 mL) and extracted with EtOAc (10 mL×3). The combined organic layers were washed with brine (10 mL), dried over Na₂SO₄, filtered and concentrated under reduced pressure to give a residue. The residue was purified by Prep-HPLC (FA condition, column: Phenomenex luna C18 100×40 mm×5 um; mobile phase: [H2O (0.2% FA)-AC□]; gradient: 25%-65% B over 8.0 min) to give the title compound (43.2 mg, 92.2 μmol, 9.89% yield) as a yellow solid. ¹H NMR (DMSO-d₆ 400 MHz) δ 8.33 (s, 2H), 7.84 (d, J=2.8 Hz, 1H), 7.61 (s, 1H), 7.44 (s, 1H), 6.18 (d, J=5.6 Hz, 1H), 5.54 (d, J=3.2 Hz, 1H), 4.78 (t, J=12.8 Hz, 1H), 3.40-3.39 (m, 4H), 2.32 (s, 3H), 1.91 (s, 3H), 1.51 (d, J=6.8 Hz, 3H), 1.45-1.47 (m, 4H), 0.98 (s, 6H).

### Example 7

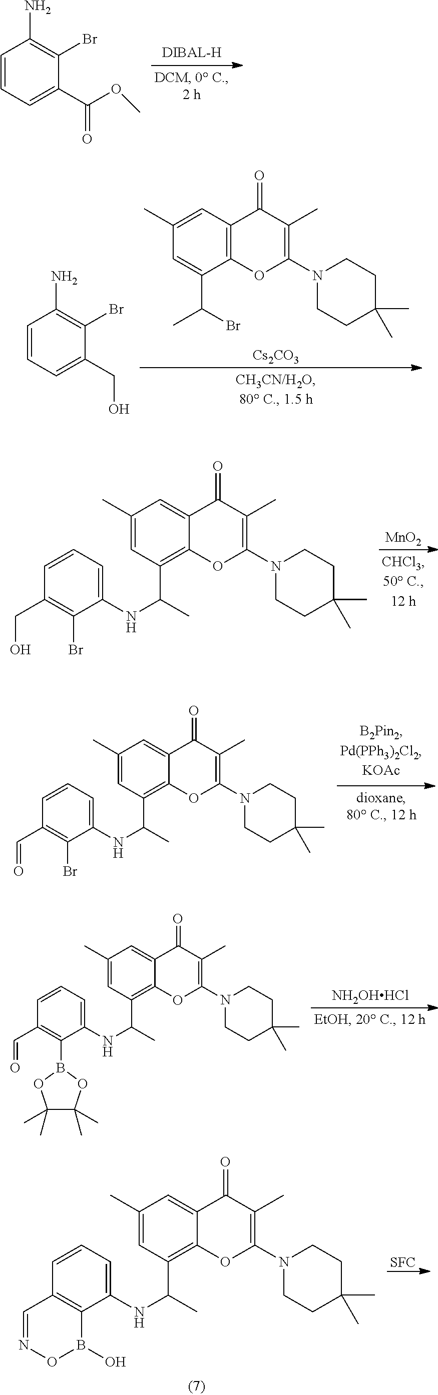

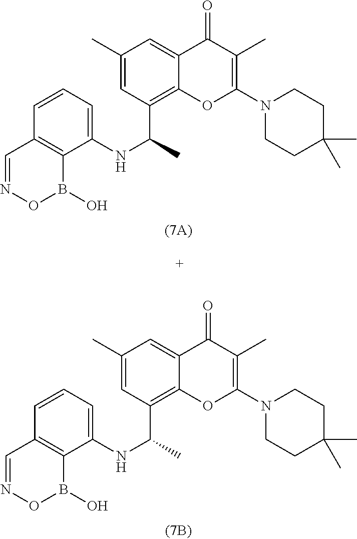 [SMILES: Cc1cc([C@@H](C)Nc2cccc3c2B(O)ON=C3)c2oc(N3CCC(C)(C)CC3)c(C)c(=O)c2c1 ; Cc1cc([C@H](C)Nc2cccc3c2B(O)ON=C3)c2oc(N3CCC(C)(C)CC3)c(C)c(=O)c2c1]

### Preparation of (3-amino-2-bromo-phenyl)methanol

To a solution of DIBAL-H (1 M in THF, 65.20 mL, 3 eq) in DCM (20 mL) was dropwise added a solution of methyl 3-amino-2-bromo-benzoate (5.00 g, 21.73 mmol, 1 eq) in DCM (10 mL) at 0° C., the mixture was stirred at 0° C. for 2 h. The reaction mixture was quenched by addition Na₂SO₄.10H₂O (20 g) at 0° C., filtered and the filtrate was concentrated under reduced pressure to give a residue. The crude product was triturated with MTBE (30 mL) at 20° C. for 30 min and filtered to give the title compound (3.30 g, 16.33 mmol, 75.15% yield) as a white solid. ¹H NMR (DMSO-d₆, 400 MHz) δ 7.04 (t, J=7.6 Hz, 1H), 6.71 (t, J=8.0 Hz, 2H), 5.24 (t, J=5.6 Hz, 3H), 4.42 (d, J=5.6 Hz, 2H).

### Preparation of 8-[1-[2-bromo-3-(hydroxymethyl)anilino]ethyl]-2-(4,4-dimethyl-1-piperidyl)-3,6-dimethyl-chromen-4-one

To a solution of 8-(1-bromoethyl)-2-(4,4-dimethyl-1-piperidyl)-3,6-dimethyl-chromen-4-one (500 mg, 1.27 mmol, 1 eq) in CH₃CN (5 mL)/H₂O (1 mL) was added (3-amino-2-bromo-phenyl)methanol (515 mg, 2.55 mmol, 2 eq) and Cs₂CO₃ (830 mg, 2.55 mmol, 2 eq) in turns at 20° C., then then reaction mixture was stirred at 80° C. for 1.5 h. After cooling to room temperature, the reaction mixture was diluted with H₂O (10 mL) and extracted with EtOAc (10 mL×3). The combined organic layers were washed with brine (10 mL×2), dried over Na₂SO₄, filtered and the filtrate was concentrated under reduced pressure to give a residue. The residue was purified by column chromatography (SiO₂, Petroleum ether/Ethyl acetate=5/1 to 1/1) to give the title compound (460 mg, 895.87 μmol, 70.30% yield) as yellow oil. ¹H NMR (DMSO-d₆, 400 MHz) δ 7.62 (s, 1H), 7.46 (d, J=2.0 Hz, 1H), 7.04 (t, J=7.6 Hz, 1H), 6.78 (d, J=7.6 Hz, 1H), 6.36 (d, J=8.0 Hz, 1H), 5.32-5.28 (m, 2H), 5.08-5.00 (m, 1H), 4.46 (d, J=5.6 Hz, 2H), 3.44-3.40 (m, 4H), 2.31 (s, 3H), 1.92 (s, 3H), 1.62 (d, J=6.8 Hz, 3H), 1.49-1.45 (m, 4H), 0.99 (s, 6H).

### Preparation of 2-bromo-3-[1-[2-(4,4-dimethyl-1-piperidyl)-3,6-dimethyl-4-oxo-chromen-8-yl]ethylamino]benzaldehyde

To a solution of 8-[1-[2-bromo-3-(hydroxymethyl)anilino]ethyl]-2-(4,4-dimethyl-1-piperidyl)-3,6-dimethyl-chromen-4-one (200 mg, 389.51 μmol, 1 eq) in CHCl₃ (2 mL) was added MnO₂ (338 mg, 3.90 mmol, 10 eq) portion-wise at 20° C., the reaction mixture was stirred at 50° C. for 12 h. After cooling to room temperature, the reaction mixture was filtered and the filtrate was concentrated under reduced pressure to give the title compound (190 mg, 371.49 μmol, 95.37% yield) as yellow oil. ¹H NMR (DMSO-d₆, 400 MHz) δ 10.25 (s, 1H), 7.64 (s, 1H), 7.46 (d, J=2.0 Hz, 1H), 7.22 (t, J=8.0 Hz, 1H), 7.08-7.05 (m, 1H), 6.73 (d, J=7.2 Hz, 1H), 5.72 (d, J=6.8 Hz, 1H), 5.13-5.06 (m, 1H), 3.43-3.39 (m, 4H), 2.31 (s, 3H), 1.92 (s, 3H), 1.66 (d, J=6.4 Hz, 3H), 1.47-1.43 (m, 4H), 0.98 (s, 6H).

### Preparation of 3-[1-[2-(4,4-dimethyl-1-piperidyl)-3,6-dimethyl-4-oxo-chromen-8-yl]ethylamino]-2-(4,4,5,5-tetramethyl-1,3,2-dioxaborolan-2-yl)benzaldehyde

To a solution of 2-bromo-3-[1-[2-(4,4-dimethyl-1-piperidyl)-3,6-dimethyl-4-oxo-chromen-8-yl]ethylamino]benzaldehyde (480 mg, 938.51 μmol, 1 eq) in dioxane (15 mL) was added B₂Pin₂ (1.19 g, 4.69 mmol, 5 eq), KOAc (276 mg, 2.82 mmol, 3 eq) and Pd(PPh₃)₂Cl₂ (66 mg, 93.85 μmol, 0.1 eq) in turns at 20° C., the reaction mixture was stirred at 80° C. for 12 h under N₂. After cooling to room temperature, the reaction mixture was filtered and the filtrate was concentrated under reduced pressure to give a residue. The residue was purified by column chromatography (SiO₂, Petroleum ether/Ethyl acetate=5/1 to 1/1) to give the title compound (490 mg, 877.33 μmol, 93.48% yield) as yellow oil. ¹H NMR (DMSO-d₆, 400 MHz) δ 10.12 (s, 1H), 7.64 (s, 1H), 7.40 (d, J=1.6 Hz, 1H), 7.30 (t, J=7.6 Hz, 1H), 7.12 (d, J=7.6 Hz, 1H), 6.65 (d, J=8.0 Hz, 1H), 5.74 (d, J=6.0 Hz, 1H), 5.08-5.01 (m, 1H), 3.38-3.35 (m, 4H), 2.32 (s, 3H), 1.91 (s, 3H), 1.57 (d, J=6.4 Hz, 3H), 1.44-1.40 (m, 4H), 1.16 (s, 12H), 0.96 (s, 6H).

### Preparation of 2-(4,4-dimethyl-1-piperidyl)-8-[1-[(1-hydroxy-2,3,1-benzoxazaborinin-8-yl)amino]ethyl]-3,6-dimethyl-chromen-4-one

To a solution of 3-[1-[2-(4,4-dimethyl-1-piperidyl)-3,6-dimethyl-4-oxo-chromen-8-yl]ethyl amino]-2-(4,4,5,5-tetramethyl-1,3,2-dioxaborolan-2-yl)benzaldehyde (440 mg, 787.80 μmol, 1 eq) in EtOH (10 mL) was added hydroxylamine; hydrochloride (109 mg, 1.58 mmol, 2 eq) in one portion at 20° C., the reaction mixture was stirred at 20° C. for 12 h. The reaction mixture was diluted with H₂O (10 mL) and the resulting suspension was directly filtered. The filter cake was triturated with CH₃CN (5 mL) and then further purified by prep-HPLC (column: Waters Xbridge BEH C18 100×30 mm×5 um; mobile phase: [H₂O (10 mM NH₄HCO₃)-AC□]; gradient: 47%-77% B over 8.0 min) to give the title compound (120 mg, 253.50 μmol, 32.18% yield) as a white solid. ¹H NMR (DMSO-d₆, 400 MHz) δ 9.78 (s, 1H), 8.44 (s, 1H), 7.64 (s, 1H), 7.44-7.39 (m, 2H), 6.84-6.81 (m, 2H), 6.51 (d, J=8.4 Hz, 1H), 5.09-5.02 (m, 1H), 3.42-3.38 (m, 4H), 2.31 (s, 3H), 1.92 (s, 3H), 1.60 (d, J=6.8 Hz, 3H), 1.47-1.43 (m, 4H), 0.97 (s, 6H). MS (ESI): mass calcd. For C₂₇H₃₂BN₃O₄ 473.25, m/z found 474.3 [M+H]⁺. HPLC: 96.51% (220 nm), 96.06% (254 nm).

### Preparation of 2-(4,4-dimethyl-1-piperidyl)-8-[(1S)-1-[(1-hydroxy-2,3,1-benzoxazaborinin-8-yl)amino]ethyl]-3,6-dimethyl-chromen-4-one and 2-(4,4-dimethyl-1-piperidyl)-8-[(1R)-1-[(1-hydroxy-2,3,1-benzoxazaborinin-8-yl)amino]ethyl]-3,6-dimethyl-chromen-4-one

2-(4,4-dimethyl-1-piperidyl)-8-[1-[(1-hydroxy-2,3,1-benzoxazaborinin-8-yl)amino]ethyl]-3,6-dimethyl-chromen-4-one (60 mg, 126.75 μmol, 1 eq) was separated by SFC (column: ChiralPak IH, 250×30 mm×10 um; mobile phase: [CO₂-EtOH]; B %: 35%, isocratic elution mode) and further purified by prep-HPLC (column: Waters Xbridge BEH C18 100×30 mm×5 um; mobile phase: [H₂O (10 mM NH₄HCO₃)-AC□]; gradient: 42%-70% B over 8.0 min) to give the title compound, Isomer 1 (10.4 mg, 21.97 μmol, 17.33% yield) as a white solid and the title compound, Isomer 2 (10.1 mg, 21.34 mol, 16.83% yield) as a white solid. Isomer 1: ¹H NMR (DMSO-d₆, 400 MHz) δ 9.79 (s, 1H), 8.44 (s, 1H), 7.64 (s, 1H), 7.43-7.39 (m, 2H), 6.84-6.82 (m, 2H), 6.50 (d, J=8.4 Hz, 1H), 5.07-5.03 (m, 1H), 3.42-3.39 (m, 4H), 2.31 (s, 3H), 1.92 (s, 3H), 1.61 (d, J=6.8 Hz, 3H), 1.46-1.44 (m, 4H), 0.97 (s, 6H). MS (ESI): mass calcd. For C₂₇H₃₂BN₃O₄ 473.25, m/z found 474.2 [M+H]⁺. HPLC: 94.77% (220 nm), 96.58% (254 nm). Chiral purity: 99.68% ee. Isomer 2: ¹H NMR (DMSO-d₆, 400 MHz) δ 9.79 (s, 1H), 8.44 (s, 1H), 7.64 (s, 1H), 7.43-7.39 (m, 2H), 6.84-6.82 (m, 2H), 6.52 (d, J=8.4 Hz, 1H), 5.07-5.04 (m, 1H), 3.42-3.39 (m, 4H), 2.31 (s, 3H), 1.92 (s, 3H), 1.61 (d, J=6.8 Hz, 3H), 1.46-1.44 (m, 4H), 0.98 (s, 6H). MS (ESI): mass calcd. For C₂₇H₃₂BN₃O₄ 473.25, m/z found 474.3 [M+H]⁺. HPLC: 93.61% (220 nm), 96.02% (254 nm). Chiral purity: 97.36% ee.

### Example 8

### Preparation of methyl 2-amino-6-bromobenzoate

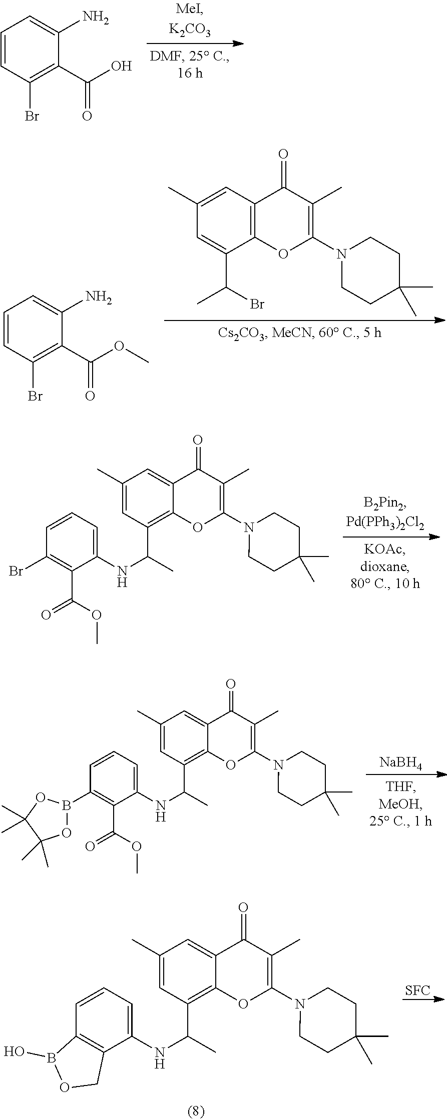

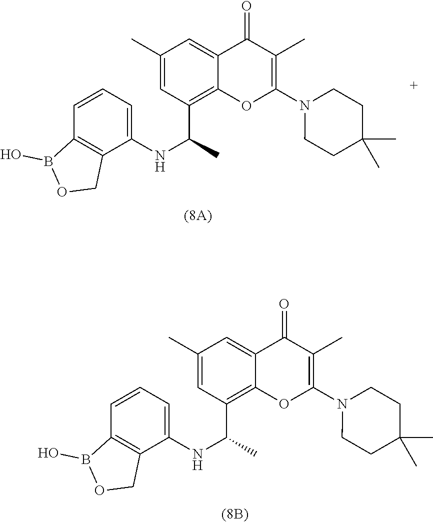 [SMILES: Cc1cc([C@@H](C)Nc2cccc3c2COB3O)c2oc(N3CCC(C)(C)CC3)c(C)c(=O)c2c1 ; Cc1cc([C@H](C)Nc2cccc3c2COB3O)c2oc(N3CCC(C)(C)CC3)c(C)c(=O)c2c1]

To a solution of 2-amino-6-bromo-benzoic acid (1.87 g, 8.66 mmol, 1 eq) and K₂CO₃ (1.44 g, 10.39 mmol, 1.2 eq) in DMF (20 mL) was added MeI (1.47 g, 10.39 mmol, 646.66 μL, 1.2 eq) dropwise at 25° C., the reaction mixture was stirred at 25° C. for 16 h. The reaction mixture was quenched by addition water (50 mL) at 25° C. and extracted with EtOAc (30 mL×3). The combined organic layers were washed with brine (50 mL×2), dried over Na₂SO₄, filtered and concentrated under reduced pressure to give a residue. The residue was purified by flash silica gel chromatography (ISCO®; 12 g SepaFlash® Silica Flash Column, Eluent of 0~3% Ethyl acetate/Petroleum ether gradient@75 m/min) to give the title compound (1.30 g, 5.65 mmol, 65.28% yield) as yellow oil. ¹H NMR (DMSO-d₆, 400 MHz) δ 7.02 (t, J=8.0 Hz, 1H), 6.76 (d, J=8.0 Hz, 1H), 6.70 (dd, J=8.0 Hz, 1H), 5.66 (s, 2H), 3.82 (s, 3H).

### Preparation of methyl 2-bromo-6-[1-[2-(4,4-dimethyl-1-piperidyl)-3,6-dimethyl-4-oxo-chromen-8-yl]ethylamino]benzoate

To a solution of methyl 2-amino-6-bromo-benzoate (500 mg, 2.17 mmol, 2 eq) and 8-(1-bromoethyl)-2-(4,4-dimethyl-1-piperidyl)-3,6-dimethyl-chromen-4-one (426 mg, 1.09 mmol, 1 eq) in MeCN (3 mL) was added Cs₂CO₃ (708 mg, 2.17 mmol, 2 eq) in portions, the reaction mixture was stirred at 60° C. for 5 h. After cooling to room temperature, the reaction mixture was quenched by adding H2O (10 mL) at 20° C. and then extracted with EtOAc (10 mL×3). The combined organic layers were washed with brine (10 mL), dried over Na₂SO₄, filtered and concentrated under reduced pressure to give a residue. The residue was purified by column chromatography (SiO₂, Petroleum ether/Ethyl acetate=3/1 to 1/1) to give the title compound (300 mg, crude) as a yellow solid, which was used directly for next step without further purification.

### Preparation of methyl 2-((1-(2-(4,4-dimethylpiperidin-1-yl)-3,6-dimethyl-4-oxo-4H-chromen-8-yl)ethyl)amino)-6-(4,4,5,5-tetramethyl-1,3,2-dioxaborolan-2-yl)benzoate

A mixture of methyl 2-bromo-6-[1-[2-(4,4-dimethyl-1-piperidyl)-3,6-dimethyl-4-oxo-chromen-8-yl]ethylamino]benzoate (150 mg, 277.02 μmol, 1 eq), B₂Pin₂ (352 mg, 1.39 mmol, 5 eq), KOAc (82 mg, 831.06 μmol, 3 eq) and Pd(PPh₃)₂Cl₂ (20 mg, 27.70 μmol, 0.1 eq) in dioxane (3 mL) was degassed and purged with N₂ for 3 times, and then the mixture was stirred at 80° C. for 10 h under N₂ atmosphere.

After cooling to room temperature, the reaction mixture was filtered and the filtrate was concentrated under reduced pressure to give a residue. The residue was purified by flash silica gel chromatography (Biotage®; 4 g SepaFlash® Silica Flash Column, Eluent of 0~20% Ethyl acetate/Petroleum ether gradient @75 mL/min) to give the title compound (150 mg, 254.87 μmol, 92.00% yield) as a white solid. ¹H NMR (DMSO-d₆, 400 MHz) δ 7.79-7.77 (d, J=10.8 Hz, 1H), 7.68 (s, 1H), 7.43 (s, 1H), 7.26 (s, 1H), 6.61-6.58 (m, 2H), 5.15-5.10 (m, 1H), 3.47-3.44 (m, 4H), 2.35 (s, 3H), 1.96 (s, 3H), 1.66 (s, 3H), 1.5 (s, 4H), 1.11 (s, 12H), 1.03 (s, 6H).

### Preparation of 2-(4,4-dimethylpiperidin-1-yl)-8-(1-((1-hydroxy-1,3-dihydrobenzo [c][1,2]oxaborol-4-yl)amino)ethyl)-3,6-dimethyl-4H-chromen-4-one

To a solution of methyl 2-[1-[2-(4,4-dimethyl-1-piperidyl)-3,6-dimethyl-4-oxo-chromen-8-yl]ethylamino]-6-(4,4,5,5-tetramethyl-1,3,2-dioxaborolan-2-yl)benzoate (230 mg, 390.80 μmol, 1 eq) in THF (3 ml) was added NaBH₄ (40 mg, 1.06 mmol, 2.71 eq) in one portion, the reaction mixture was stirred at 25° C. for 1 h. The reaction mixture was quenched by addition sat.aq NH₄Cl (15 mL) at 0° C. and extracted with EA (15 mL×3). The combined organic layers were washed with brine (15 mL), dried over Na₂SO₄, filtered and concentrated under reduced pressure to give a residue. The residue was purified by prep-HPLC (basic condition; column: Phenomenex Gemini □XC₁₈ (75×30 mm×3 um); mobile phase: [H₂O (0.05% NH₃H₂O+10 mM NH₄HCO₃)-AC□]; gradient: 30%-70% B over 8.0 min) to give the title compound (40 mg, 84.95 μmol, 21.74% yield) as a white solid. ¹H NMR (DMSO-d₆, 400 MHz) δ 8.98 (s, 1H), 7.60 (s, 1H), 7.47 (d, J=1.9 Hz, 1H), 7.04-6.84 (m, 2H), 6.30 (d, J=7.8 Hz, 1H), 5.68 (d, J=6.9 Hz, 1H), 5.10-4.87 (m, 3H), 3.42 (d, J=2.0 Hz, 4H), 2.29 (s, 3H), 1.92 (s, 3H), 1.55 (d, J=6.8 Hz, 3H), 1.47 (d, J=3.6 Hz, 4H), 0.98 (s, 6H). MS (ESI): mass calcd. For C₂₇H₃₃BN₂O₄ 460.25 m/z found 461.3 [M+H]⁺. HPLC: 97.77% (220 nm), 99.24% (254 nm).

### Preparation of (S)-2-(4,4-dimethylpiperidin-1-yl)-8-(1-((1-hydroxy-1,3-dihydrobenzo [c][1,2]oxaborol-4-yl)amino)ethyl)-3,6-dimethyl-4H-chromen-4-one and (R)-2-(4,4-dimethylpiperidin-1-yl)-8-(1-((1-hydroxy-1,3-dihydrobenzo[c][1,2]oxaborol-4-yl)amino)ethyl)-3,6-dimethyl-4H-chromen-4-one

2-(4,4-dimethyl-1-piperidyl)-8-[1-[(1-hydroxy-3H-2,1-benzoxaborol-4-yl)amino]ethyl]-3,6-dimethylchromen-4-one (40 mg) was separated by SFC (column: ChiralPak IH, 250×30 mm×10 um; mobile phase: [CO₂-EtOH (0.1% NH₃·H₂O)]; B %: 40%, isocratic elution mode) to give the title compound, Isomer 1 (18.3 mg, 39.75 μmol, 45.75% yield) as a white solid and the title compound, Isomer 2 (16.1 mg, 34.97 μmol, 40.25% yield) as a white solid. Isomer 1: ¹H NMR (DMSO-d₆, 400 MHz) δ 8.97 (s, 1H), 7.60 (s, 1H), 7.47 (d, J=2.0 Hz, 1H), 6.98-6.96 (m, 1H), 6.93-6.91 (m, 1H), 6.30 (d, J=7.6 Hz, 1H), 5.67 (d, J=6.8 Hz, 1H), 5.05-4.90 (m, 3H), 3.43 (s, 4H), 2.29 (s, 3H), 1.93 (s, 3H), 1.55 (d, J=6.8 Hz, 3H), 1.47-1.46 (m, 4H), 0.99 (s, 6H). MS (ESI): mass calcd. For C₂₇H₃₃BN₂O₄ 460.25 m/z found 461.2 [M+H]⁺. HPLC: 98.73% (220 nm), 100.00% (254 nm). Chiral purity: 100% ee. Isomer 2: ¹H NMR (DMSO-d₆, 400 MHz) δ 8.97 (s, 1H), 7.60 (s, 1H), 7.47 (d, J=1.6 Hz, 1H), 6.98-6.96 (m, 1H), 6.93-6.91 (m, 1H), 6.31 (d, J=7.6 Hz, 1H), 5.67 (d, J=6.8 Hz, 1H), 5.05-4.90 (m, 3H), 3.43-3.41 (s, 4H), 2.29 (s, 3H), 1.93 (s, 3H), 1.55 (d, J=6.8 Hz, 3H), 1.47-1.46 (m, 4H), 0.99 (s, 6H). MS (ESI): mass calcd. For C₂₇H₃₃BN₂O₄ 460.25 m/z found 461.2 [M+H]⁺. HPLC: 99.75% (220 nm), 100.00% (254 nm). Chiral purity: 99.90% ee.

### Example 9

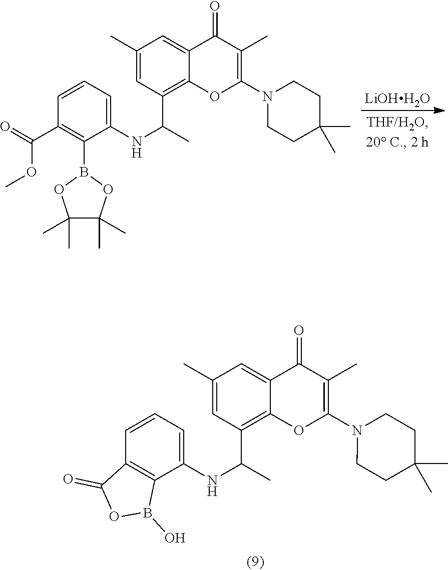

### Preparation of 7-[1-[2-(4,4-dimethyl-1-piperidyl)-3,6-dimethyl-4-oxo-chromen-8-yl]ethylamaino]-1-hydroxy-2,1-benzoxaborol-3-one

A mixture of methyl 3-[1-[2-(4,4-dimethyl-1-piperidyl)-3,6-dimethyl-4-oxo-chromen-8-yl]ethylamino]-2-(4,4,5,5-tetramethyl-1,3,2-dioxaborolan-2-yl)benzoate (350 mg, 595 μmol, 1 eq) and LiOH·H₂O (249 mg, 5.95 mmol, 10 eq) in THF (2 mL)/H₂O (1 mL) was stirred at 20° C. for 2 h. The reaction mixture was concentrated in vacuum and the residue was poured into ice-water (w/w=1/1) (10 mL). The aqueous phase was acidified to pH=7 with HCl (2N) and extracted with EtOAc (20 mL×3). The combined organic phase was washed with brine (10 mL), dried with anhydrous Na₂SO₄, filtered and concentrated in vacuum. The residue was purified by prep-HPLC (column: Waters Xbridge BEH C18 100×30 mm×10 um; mobile phase: [H₂O (10 mM NH₄HCO₃)-AC□]; gradient: 10%-40% B over 8.0 min) to give the title compound (80.4 mg, 159.36 μmol, 26.80% yield) as a white solid. ¹H NMR (DMSO+D₂O-d₆, 400 MHz) δ 7.59 (s, 1H), 7.54 (s, 1H), 6.81-6.77 (m, 1H), 6.72-6.70 (m, 1H), 6.04 (d, J=7.2 Hz, 1H), 4.95-4.91 (m, 1H), 3.45 (s, 4H), 2.25 (s, 3H), 1.89 (s, 3H), 1.48-1.43 (m, 7H), 0.93 (d, J=7.2 Hz, 6H). MS (ESI): mass calcd. For C₂₇H₃₁BN₂O₅ 474.23, m/z found 475.2 [M+H]⁺. HPLC: 94.17% (220 nm), 95.27% (254 nm).

### Example 10

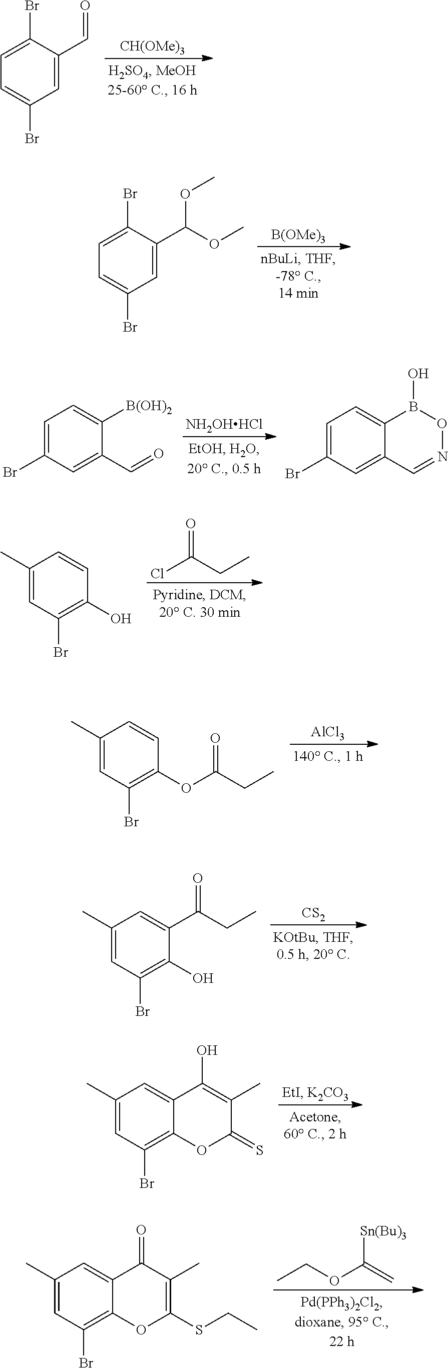

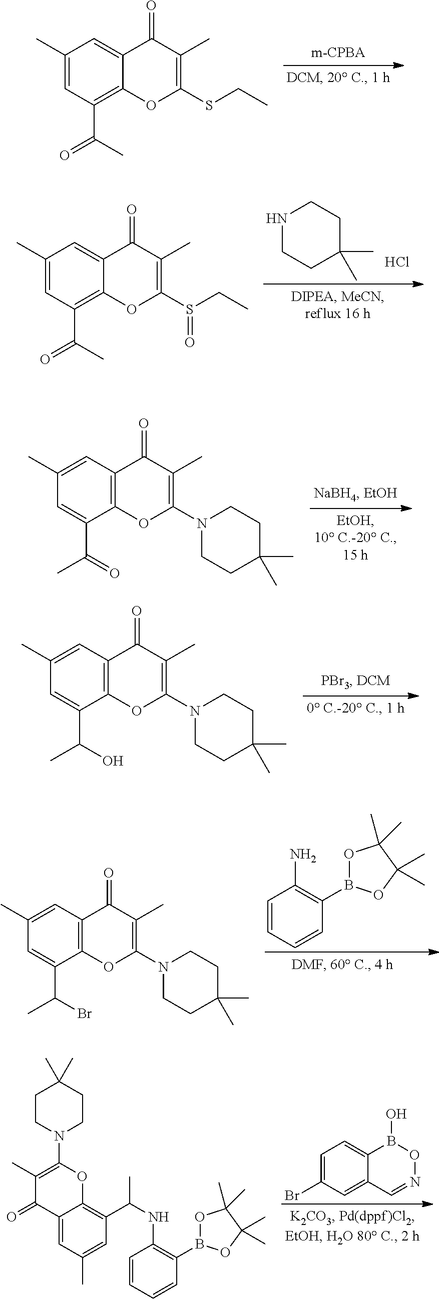

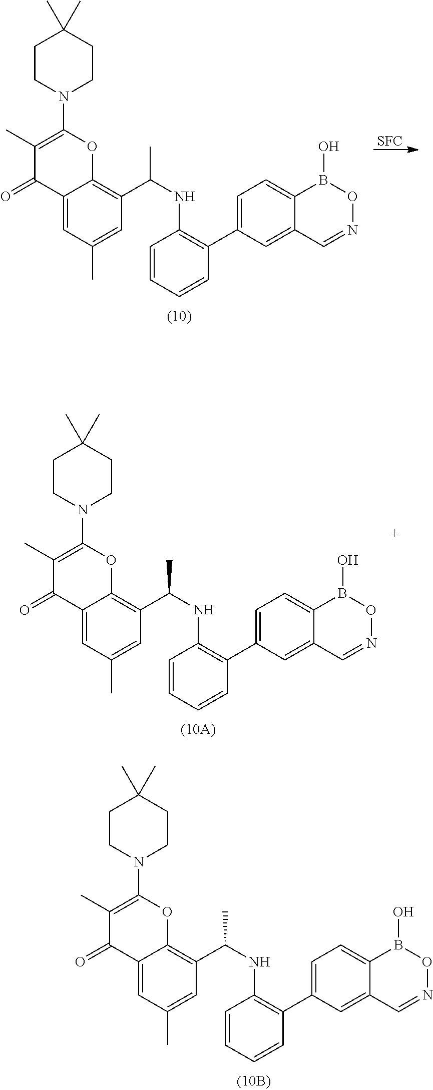

### Preparation of 1,4-dibromo-2-(dimethoxymethyl)benzene

To a solution of 2,5-dibromobenzaldehyde (100 g, 378.91 mmol, 1 eq) and trimethoxymethane (80.42 g, 757.82 mmol, 83.08 mL, 2 eq) in MeOH (1000 mL) was added H₂SO₄ (1.11 g, 11.37 mmol, 0.03 eq) (3 drops) dropwise at 20° C., the mixture was heated to 60° C. and stirred at 60° C. for 16 h. 3 parallel reactions were combined for work up. The combined reaction mixture was cooled to 20° C., adjusted to pH=8 with MeONa (30% in MeOH) and concentrated under reduced pressure. The residue was dissolved in EtOAc (1500 mL), washed with H₂O (1500 mL) and brine, dried over Na₂SO₄, filtered and concentrated under reduced pressure to give the title compound (310 g, 1 μmol, 88.0% yield) as light yellow oil. ¹H NMR (CDCl₃, 400 MHz) δ 7.75 (d, J=2.4 Hz, 1H), 7.42 (d, J=8.4 Hz, 1H), 7.33 (dd, J=8.4, 2.4 Hz, 1H), 5.51 (s, 1H), 3.39 (s, 6H).

### Preparation of (4-bromo-2-formylphenyl)boronic acid

To a solution of 1,4-dibromo-2-(dimethoxymethyl)benzene (130 g, 419.38 mmol, 1 eq) in THF (1300 mL) was added n-BuLi (2.5 M, 167.8 mL, 1 eq) dropwise at −78° C., the mixture was stirred at −78° C. for 15 min. Then B(OMe)₃ (66 g, 629.07 mmol, 71.05 mL, 1.5 eq) was added dropwise at −78° C. and the mixture was stirred at −78° C. for 30 min. 2 parallel reactions were combined for work up. The combined mixtures were allowed to warm to 0° C., quenched with aq.NH₄Cl (600 mL) at 0° C. and extracted with EtOAc (1000 mL×2). The combined organic layers were washed with brine, dried over Na₂SO₄ and concentrated under reduced pressure. The residue was dissolved in THF (3000 mL) and treated with HCl (4 N, 4.08 μL, 21 eq). The resulting mixture was stirred at 20° C. for 15 min and then directly extracted with EtOAc (1000 mL×2). The combined organic layers were washed with brine, dried over Na₂SO₄ and concentrated under reduced pressure to give the title compound (150 g, crude) as a light yellow solid. The product was used directly for next step without further purification.

### Preparation of 6-bromo-1-hydroxy-2,3,1-benzoxazaborinine

To a mixture of (4-bromo-2-formyl-phenyl)boronic acid (150 g, 655.49 mmol, 1 eq) in EtOH (3000 mL) was added a solution of NH₂OH·HCl (91 g, 1.31 μmol, 2 eq) in H₂O (400 mL) dropwise at 20° C., the mixture was stirred at 20° C. for 0.5 h. The mixture was diluted with H₂O (400 mL) at 20° C. and the resulting suspension was directly filtered. The filter cake was washed with H₂O (100 mL) and then dried under reduced pressure to give the title compound (60 g, 265.68 mmol, 31.7% yield by 2 steps) as an off-white solid. ¹H NMR (DMSO-d₆, 400 MHz) δ 9.55 (s, 1H), 8.64 (s, 1H), 8.04 (d, J=2.0 Hz, 1H), 8.01 (d, J=8.0 Hz, 1H), 7.93 (dd, J=7.6, 1.6 Hz, 1H).

### Preparation of (2-bromo-4-methyl-phenyl)propanoate

To a solution of 2-bromo-4-methyl-phenol (900.00 g, 4.81 μmol, 1 eq) and Pyridine (570.94 g, 7.22 μmol, 582.59 mL, 1.5 eq) in DCM (2000 mL) was added dropwise propanoyl chloride (489.74 g, 5.29 mol, 489.74 mL, 1.1 eq) at 0° C., the mixture was warmed to 20° C. and stirred at 20° C. for 0.5 h. The reaction mixture (combined with 100 g batch) was poured into water (1000 mL), adjusted to pH=2 by HCl (3 N) and then extracted with DCM (1000 mL×3). The combined organic layers were washed with brine, dried over Na₂SO₄, filtered and concentrated under reduced pressure to give the title compound (1300 g, crude) as light yellow oil. ¹H NMR (DMSO-d₆, 400 MHz) δ 7.52 (s, 1H), 7.22 (dd, J=1.2, 8.4 Hz, 1H), 7.14 (d, J=8.4 Hz, 1H), 2.62 (q, J=7.6 Hz, 2H), 2.30 (s, 3H), 1.17 (t, J=7.6 Hz, 3H).

### Preparation of 1-(3-bromo-2-hydroxy-5-methyl-phenyl) propan-1-one

A mixture of (2-bromo-4-methyl-phenyl) propanoate (300 g, 1.23 μmol, 1 eq) and AlCl₃ (329.10 g, 2.47 μmol, 2 eq) was heated to 140° C. and stirred at 140° C. for 1 h. 5 reactions were combined for work up. The combined reaction mixture was cooled to 20° C., poured into ice water (5000 mL) and then extracted with EtOAc (2000 mL×3). The combined organic layers were washed with brine, dried over anhydrous Na₂SO₄ and concentrated in vacuo. The crude product was triturated with PE and EtOAc (PE/EtOAc=100/1; 2 L) at 20° C. for 30 min to give the title compound (900 g, 3.70 μmol, 69.2% yield by 2 steps) as a brown solid. ¹H NMR (DMSO-d₆, 400 MHz) δ 12.66 (s, 1H), 7.81 (d, J=1.2 Hz, 1H), 7.71 (d, J=1.2 Hz, 1H), 3.15 (q, J=7.2 Hz, 2H), 2.28 (s, 3H), 1.10 (t, J=7.2 Hz, 3H).

### Preparation of 8-bromo-4-hydroxy-3, 6-dimethyl-chromene-2-thione

To a solution of 1-(3-bromo-2-hydroxy-5-methyl-phenyl)propan-1-one (300 g, 1.23 μmol, 1 eq) in THF (3000 mL) was added KOtBu (415 g, 3.70 μmol, 3 eq) in portions at 0° C., the mixture was stirred at 0° C. for 10 min. Then CS₂ (141 g, 1.85 μmol, 111.60 mL, 1.5 eq) was added dropwise at 0° C., the resulting mixture was warmed to 20° C. and stirred at 20° C. for 0.5 h. 4 reaction was combined for work up. The combined mixture was poured into water (5000 mL), adjusted to pH=4 by HCl (2 N) and extracted with EtOAc (1500 mL×3). The combined organic layers were washed with brine, dried over Na₂SO₄, filtered and concentrated under reduced pressure to give a residue. The crude product was triturated with DCM (6 L) at 20° C. for 10 min to give the title compound (900 g, 3.16 μmol, 76.7% yield) as a yellow solid. ¹H NMR (DMSO-d₆, 400 MHz) δ 7.81 (s, 1H), 7.78 (s, 1H), 2.39 (s, 3H), 2.26 (s, 3H).

### Preparation of 8-bromo-2-ethylsulfanyl-3,6-dimethyl-chromen-4-one

To a mixture of 8-bromo-4-hydroxy-3,6-dimethyl-chromene-2-thione (200 g, 701.37 mmol, 1 eq) and K₂CO₃ (116 g, 841.64 mmol, 1.2 eq) in Acetone (2500 mL) was added EtI (459 g, 2.95 μmol, 235.6 mL, 4.2 eq) dropwise at 20° C., the mixture was heated to 60° C. and stirred at 60° C. for 2 h. 5 reactions were combined for work up. The combined reaction mixture was cooled to 20° C. and then the resulting suspension was directly filtered. The filtrate was diluted with EtOAc (5000 mL), washed with H₂O (2500 mL×2) and then brine (1000 mL). The organic layer was dried over Na₂SO₄, filtered and concentrated under reduced pressure. The residue was triturated with PE (2 L) to give the title compound (900 g, 2.87 mol, 91.0% yield) as an orange solid. ¹H NMR (DMSO-d₆, 400 MHz) δ 7.88 (d, J=1.6 Hz, 1H), 7.77 (d, J=1.6 Hz 1H), 3.30 (q, J=7.2 Hz, 2H), 2.40 (s, 3H), 1.92 (s, 3H), 1.42 (t, J=7.2 Hz, 3H).

### Preparation of 8-acetyl-2-ethylsulfanyl-3, 6-dimethyl-chromen-4-one

To a solution of 8-bromo-2-ethylsulfanyl-3,6-dimethyl-chromen-4-one (200 g, 638.55 mmol, 1 eq) in dioxane (2000 mL) was added tributyl(1-ethoxyvinyl)stannane (270 g, 747.10 mmol, 252.40 mL, 1.17 eq) dropwise at 20° C., then the mixture was stirred at 20° C. for 10 min. Then Pd(PPh₃)₂Cl₂ (31.37 g, 44.70 mmol, 0.07 eq) was added in portions to the mixture above at 20° C. and then the resulting mixture was heated to 95° C. and stirred at 95° C. for 22 h under N₂. The reaction mixture was cooled to 20° C., added HCl (200 mL, 2 N) dropwise, and then the mixture was stirred at 50° C. for 0.5 h. 6 reactions were combined for work up. The combined reaction mixture was cooled to 20° C., diluted with sat.aq.KF (4000 mL) and then the resulting suspension was directly filtered. The filter cake was washed with EtOAc (1500 mL×3) and the filtrate was extracted with EtOAc (1500 mL×3). The combined organic phases were washed with brine, dried over Na₂SO₄, filtered and concentrated under reduced pressure. The residue was purified by column chromatography (SiO₂, Petroleum ether/Ethyl acetate=5/1 to 2/1) to give the title compound (700 g, 66.1% yield) as a yellow solid. ¹H NMR (DMSO-d₆, 400 MHz) δ 8.01 (s, 2H), 3.30 (q, J=7.2 Hz, 2H), 2.68 (s, 3H), 2.46 (s, 3H), 1.95 (s, 3H), 1.34 (t, J=7.2 Hz, 3H).

### Preparation of 8-acetyl-2-ethylsulfinyl-3,6-dimethyl-chromen-4-one

To a solution of 8-acetyl-2-ethylsulfanyl-3,6-dimethyl-chromen-4-one (200 g, 723.72 mmol, 1 eq) in DCM (2000 mL) was added m-CPBA (161.62 g, 796.09 mmol, 1.1 eq) in portions at 0° C., the mixture was warmed to 20° C. and stirred at 20° C. for 1 h. 5 reactions were combined for work up. The combined reaction mixture was filtered to give a filtrate. The filtrate was poured into sat.aq.Na₂SO₃ (6000 mL) and then extracted with DCM (2000 mL×3). The combined organic layers were washed with brine, dried over Na₂SO₄, filtered and concentrated under reduced pressure to give a residue. The crude product was triturated with MTBE (10 L) to give the title compound (423 g, 48.6% yield) as a yellow solid. ¹H NMR (DMSO-d₆, 400 MHz) δ 8.06 (d, J=1.6 Hz, 1H), 7.99 (d, J=2.4 Hz, 1H), 3.26 (q, J=7.6 Hz, 2H), 2.80 (s, 3H), 2.46 (s, 3H), 2.08 (s, 3H), 1.23 (t, J=7.6 Hz, 3H). MS (ESI): mass calcd. For C₁₅H₁₆O₄S 292.08, m/z found 292.9 [M+H]⁺. HPLC: 84.94% (220 nm), 84.43% (254 nm).

### Preparation of 8-acetyl-2-(4,4-dimethyl-1-piperidyl)-3,6-dimethyl-chromen-4-one

To a solution of 8-acetyl-2-ethylsulfinyl-3,6-dimethyl-chromen-4-one (130.00 g, 444.67 mmol, 1.0 eq) and 4,4-dimethylpiperidine hydrochloride (99.83 g, 667.01 mmol, 1.5 eq) in MeCN (1300 mL) was added DIPEA (172.41 g, 1.33 μmol, 232.36 mL, 3 eq) dropwise at 20° C., the mixture was stirred under reflux for 16 h. 2 parallel reaction were combined for work up. The reaction mixture was cooled to 20° C. and directly filtered. The filter cake was washed by MeCN (300 mL) and dried in vacuo. The crude product was then triturated with MTBE (500 mL) at 20° C. to give the title compound (230 g, 702.47 mmol, 79.0% yield) as yellow solid. ¹H NMR (CDCl₃, 400 MHz) δ 8.16 (d, J=2.0 Hz, 1H), 7.77 (d, J=2.4 Hz, 1H), 3.44-3.41 (m, 4H), 2.71 (s, 3H), 2.45 (s, 3H), 2.04 (s, 3H), 1.53-1.50 (m, 4H), 1.03 (s, 6H).

### Preparation of 2-(4,4-dimethyl-1-piperidyl)-8-(1-hydroxyethyl)-3,6-dimethyl-chromen-4-one

To a solution of 8-acetyl-2-(4,4-dimethyl-1-piperidyl)-3,6-dimethyl-chromen-4-one (20.00 g, 61.1 mmol, 1 eq) in EtOH (200 mL) was added NaBH₄ (2.68 g, 70.8 mmol, 1.16 eq) in portions at 10° C. over 20 min under N₂, then the reaction mixture was warmed to 20° C. and stirred at 20° C. for 15 h. The reaction mixture was poured into ice water (300 mL) at 0° C. and extracted with DCM (120 mL×3). The combined organic phase was washed with brine (200 mL), dried over Na₂SO₄, filtered and concentrated under reduced pressure to give the title compound (20.0 g, 60.71 mmol, 99.39% yield) as a yellow solid, which was used into the next step without further purification. ¹H NMR (DMSO-d₆, 400 MHz) δ 7.62 (s, 1H), 7.57 (d, J=2.0 Hz, 1H), 5.32 (d, J=4.4 Hz, 1H), 5.25-5.18 (m, 1H), 3.40-3.34 (m, 4H), 2.39 (s, 3H), 1.89 (s, 3H), 1.46 (t, J=5.6 Hz, 4H), 1.40 (d, J=6.4 Hz, 3H), 0.99 (s, 6H).

### Preparation of 8-(1-bromoethyl)-2-(4,4-dimethyl-1-piperidyl)-3,6-dimethyl-chromen-4-one

To a solution of 2-(4,4-dimethyl-1-piperidyl)-8-(1-hydroxyethyl)-3,6-dimethyl-chromen-4-one (10.00 g, 30.4 mmol, 1 eq) in DCM (100 mL) was added dropwise PBr₃ (9.86 g, 36.4 mmol, 3.46 mL, 1.2 eq) at 0° C. under N₂, then the reaction mixture was warmed to 20° C. and stirred 20° C. for 1 h. 2 parallel reactions were combined for work up. The reaction mixture was quenched by sat.aq NaHCO₃ (200 mL) at 0° C., adjusted pH=7 with sat.aq NaHCO₃ and extracted with DCM (150 mL×3). The combined organic layers were washed with brine (200 mL×2), dried over Na2SO₄, filtered and concentrated under reduced pressure to give a residue. The residue was purified by flash silica gel chromatography (Biotage®; 220 g SepaFlash® Silica Flash Column, Eluent of 0~20% Ethyl acetate/Petroleum ether gradient@100 mL/min) to give the title compound (11.20 g, 28.55 mmol, 47.02% yield) as a white solid.

¹H NMR (CDCl₃, 400 MHz) δ 7.95 (s, 1H), 7.52 (d, J=2.0 Hz, 1H), 5.68 (q, J=7.2 Hz, 1H), 3.49-3.45 (m, 4H), 2.45 (s, 3H), 2.15 (d, J=7.2H, 3H), 2.05 (s, 3H), 1.57-1.53 (m, 4H), 1.06 (s, 6H).

### Preparation of 2-(4,4-dimethyl-1-piperidyl)-3,6-dimethyl-8-[1-[2-(4,4,5,5-tetramethyl-1,3,2-dioxaborolan-2-yl)anilino]ethyl]chromen-4-one

A solution of 8-(1-bromoethyl)-2-(4,4-dimethyl-1-piperidyl)-3,6-dimethyl-chromen-4-one (0.50 g, 1.27 mmol, 1.00 eq) and 2-(4,4,5,5-tetramethyl-1,3,2-dioxaborolan-2-yl)aniline (558 mg, 2.55 mmol, 2.00 eq) in DMF (5 mL) was stirred at 60° C. for 4 h. The reaction mixture was cooled to 20° C., diluted with water (20 mL) and then extracted with MTBE (10 mL×3). The combined organic phase was washed with brine (10 mL×2), dried over Na₂SO₄, filtered and concentrated under reduced pressure to give the title compound (0.70 g, crude) as a brown solid, which was used directly for next step without further purification.

### Preparation of 2-(4,4-dimethyl-1-piperidyl)-8-[1-[2-(1-hydroxy-2,3,1-benzoxaza borinin-6-yl)anilino]ethyl]-3,6-dimethyl-chromen-4-one

A mixture of 6-bromo-1-hydroxy-2,3,1-benzoxazaborinine (250 mg, 1.11 mmol, 1.00 eq), 2-(4,4-dimethyl-1-piperidyl)-3,6-dimethyl-8-[1-[2-(4,4,5,5-tetramethyl-1,3,2-dioxaborolan-2-yl)anilino]ethyl]chromen-4-one (587 mg, 1.11 mmol, 1.00 eq), K₂CO₃ (306 mg, 2.21 mmol, 2.00 eq) and Pd(dppf)Cl₂ (81 mg, 110.70 μmol, 0.10 eq) in EtOH (5 mL)/H₂O (2 mL) was degassed and purged with N₂ for 3 times, and then the mixture was stirred at 80° C. for 2 h under N₂ atmosphere. The reaction mixture was cooled to 20° C., filtered through a pad of Celite and the filter cake was washed with EtOH (10 mL). The filtrate was concentrated under reduced pressure. The crude product was purified by reversed-phase HPLC (column: Waters Xbridge BEH C18 250×50 mm×10 um; mobile phase: [H₂O (10 mM NH₄HCO₃)-AC□]; gradient:30%-60% B over 8.0 min) to give the title compound (180 mg, 312.88 μmol, 28.26% yield) as a white solid. 1H NMR (DMSO-d₆, 400 MHz) δ 9.37 (s, 1H), 8.65 (s, 1H), 8.15 (d, J=7.6 Hz, 1H), 7.84-7.82 (m, 2H), 7.61 (s, 1H), 7.48 (s, 1H), 7.11-7.04 (s, 1H), 6.71 (t, J=7.2 Hz, 1H), 6.56 (d, J=8.0 Hz, 1H), 5.05 (d, J=6.4 Hz, 1H), 4.95-4.91 (m, 1H), 3.31-3.27 (m, 4H), 2.32 (s, 3H), 1.90 (s, 3H), 1.47 (d, J=6.0 Hz, 3H), 1.39-1.35 (m, 4H), 0.95 (s, 6H). MS (ESI): mass calcd. For C₃₃H₃₆BN₃O₄ 549.28, m/z found 550.2 [M+H]⁺. HPLC: 95.51% (220 nm), 96.98% (254 nm).

### Preparation of 2-(4,4-dimethyl-1-piperidyl)-8-[(R)-1-[2-(1-hydroxy-2,3,1-benzoxazaborinin-6-yl)anilino]ethyl]-3,6-dimethyl-chromen-4-one and 2-(4,4-dimethyl-1-piperidyl)-8-[(1S)-1-[2-(1-hydroxy-2,3,1-benzoxazaborinin-6-yl)anilino]ethyl]-3,6-dimethyl-chromen-4-one

2-(4,4-dimethyl-1-piperidyl)-8-[1-[2-(1-hydroxy-2,3,1-benzoxazaborinin-6-yl)anilino]ethyl]-3,6-dimethyl-chromen-4-one (180 mg, 309.39 μmol, 1.00 eq) was separated by SFC (column: DAICEL CHIRALCEL OD (250 mm×30 mm×10 um); mobile phase: [C₀₂-EtOH (0.1% NH₃H₂O)]; B %:50%, isocratic elution mode) to give the title compound, Isomer 1 (61.9 mg, 111.30 μmol, 35.97% yield) as a white solid and the title compound, Isomer 2 (77.6 mg, 140.38 μmol, 45.37% yield) was obtained as a white solid. Isomer 1: ¹H NMR (DMSO-d₆, 400 MHz) δ 9.43 (s, 1H), 8.66 (s, 1H), 8.15 (d, J=7.6 Hz, 1H), 7.85-7.82 (m, 2H), 7.60 (s, 1H), 7.48 (d, J=2.0 Hz, 1H), 7.09-7.07 (m, 1H), 7.04 (d, J=1.2 Hz, 1H), 6.71 (t, J=7.6 Hz, 1H), 6.56 (d, J=8.4 Hz, 1H), 5.06 (d, J=6.8 Hz, 1H), 4.96-4.91 (m, 1H), 3.31-3.22 (m, 4H), 2.32 (s, 3H), 1.90 (s, 3H), 1.47 (d, J=6.8 Hz, 3H), 1.40-1.38 (m, 4H), 0.95 (s, 6H). MS (ESI): mass calcd. For C₃₃H₃₆BN₃O₄ 549.28, m/z found 550.2 [M+H]⁺. HPLC: 98.80% (220 nm), 100.00% (254 nm). Chiral purity: 100% ee. Isomer 2: ¹H NMR (DMSO-d₆, 400 MHz) δ 9.43 (s, 1H), 8.66 (s, 1H), 8.15 (d, J=7.6 Hz, 1H), 7.85-7.82 (m, 2H), 7.60 (s, 1H), 7.48 (d, J=1.2 Hz, 1H), 7.09-7.06 (m, 1H), 7.04 (d, J=1.2 Hz, 1H), 6.71 (t, J=7.6 Hz, 1H), 6.56 (d, J=8.4 Hz, 1H), 5.06 (d, J=6.8 Hz, 1H), 4.96-4.90 (m, 1H), 3.31-3.27 (m, 4H), 2.32 (s, 3H), 1.90 (s, 3H), 1.47 (d, J=6.8 Hz, 3H), 1.40-1.35 (m, 4H), 0.95 (s, 6H). MS (ESI): mass calcd. For C₃₃H₃₆BN₃O₄ 549.28, m/z found 550.3 [M+H]⁺. HPLC: 99.41% (220 nm), 100.00% (254 nm). Chiral purity: 99.16% ee.

### Example 11

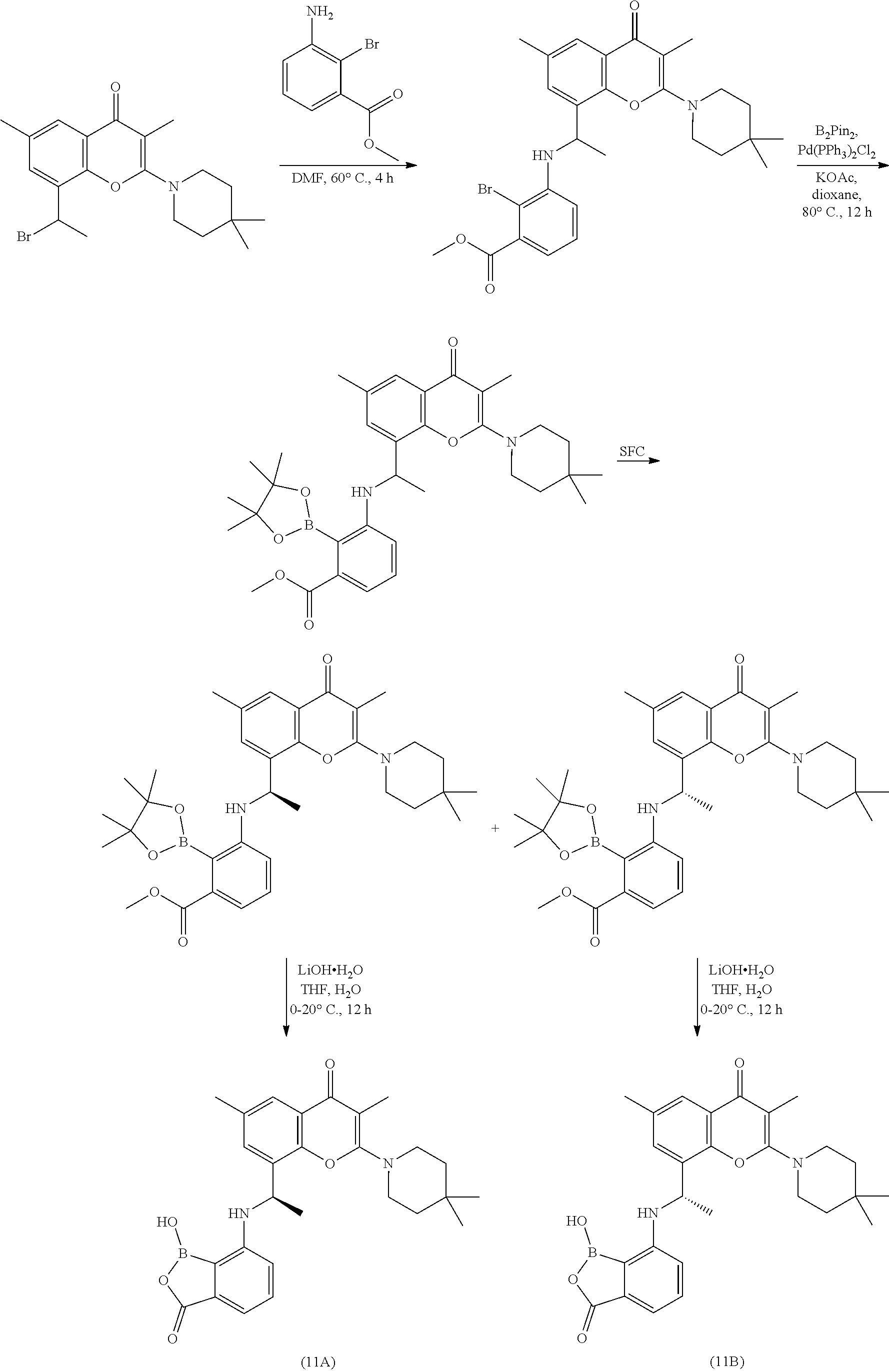

### Preparation of methyl 2-bromo-3-[1-[2-(4,4-dimethyl-1-piperidyl)-3,6-dimethyl-4-oxo-chromen-8-yl]ethylamino]benzoate

To a solution of 8-(1-bromoethyl)-2-(4,4-dimethyl-1-piperidyl)-3,6-dimethyl-chromen-4-one (500 mg, 1.27 mmol, 1 eq) in DMF (5 mL) was added methyl 3-amino-2-bromo-benzoate (498 mg, 2.17 mmol, 1.7 eq) at 25° C. in one portion, the reaction mixture was stirred at 60° C. for 4 h. The reaction mixture was cooled to 0° C. and quenched by adding water (10 mL), and then extracted with EtOAc (10 mL×3). The combined organic layers were washed with brine (10 mL×2), dried over Na₂SO₄, filtered and concentrated under reduced pressure to give a residue. The residue was purified by flash silica gel chromatography (Biotage®; 20 g SepaFlash® Silica Flash Column, Eluent of 0~40% Ethyl acetate/Petroleum ether gradient@50 mL/min) to give the title compound (450 mg, 831.06 mol, 65.21% yield) as light yellow solid. ¹H NMR (CDCl₃, 400 MHz) δ 7.88 (s, 1H), 7.32 (s, 1H), 7.07-7.03 (m, 1H), 7.00-6.98 (m, 1H), 6.41 (dd, J=8.0 Hz, 1.2 Hz, 1H), 5.11 (d, J=5.6 Hz, 1H), 5.02-4.98 (m, 1H), 3.93 (s, 3H), 3.42-3.39 (m, 4H), 2.36 (s, 3H), 2.06 (s, 3H), 1.68 (d, J=6.8 Hz, 3H), 1.58 (s, 1H), 1.54-1.51 (m, 4H), 1.04 (s, 6H).

### Preparation of methyl 3-[1-[2-(4,4-dimethyl-1-piperidyl)-3,6-dimethyl-4-oxo-chromen-8-yl]ethylamino]-2-(4,4,5,5-tetramethyl-1,3,2-dioxaborolan-2-yl)benzoate

A mixture of methyl 2-bromo-3-[1-[2-(4,4-dimethyl-1-piperidyl)-3,6-dimethyl-4-oxo-chromen-8-yl]ethylamino]benzoate (450 mg, 831.06 μmol, 1 eq), B₂Pin₂ (1.06 g, 4.16 mmol, 5 eq), KOAc (245 mg, 2.49 mmol, 3 eq) and Pd(PPh₃)₂Cl₂ (58 mg, 83.11 μmol, 0.1 eq) in dioxane (10 mL) was degassed and purged with N₂ for 3 times at 25° C., and then the mixture was heated to 80° C. and stirred at 80° C. for 12 h under N₂ atmosphere. The reaction mixture was cooled to 25° C., filtered and the filtrate was concentrated under reduced pressure to give a residue. The residue was purified by flash silica gel chromatography (Biotage®; 20 g SepaFlash® Silica Flash Column, Eluent of 0-35% Ethyl acetate/Petroleum ether gradient@60 mL/min). The eluent was concentrated under reduced pressure to give a residue. The residue was purified by prep-HPLC (column: PhenomenexGemini-QX 80×40 mm×3 um; mobile phase: [H₂O (10 mM NH₄HCO₃)-AC□]; gradient: 50%-90% B over 8.0 min) to give the title compound (250 mg, crude) as light yellow oil. ¹H NMR (DMSO-d₆, 400 MHz) δ 7.64 (s, 1H), 7.39 (s, 1H), 7.18 (t, J=8.0 Hz, 1H), 7.02 (d, J=7.6 Hz, 1H), 6.55 (d, J=8.4 Hz, 1H), 5.21 (d, J=6.0 Hz, 1H), 5.04-5.01 (m, 1H), 3.78 (s, 3H), 3.37-3.34 (m, 4H), 2.31 (s, 3H), 1.91 (s, 3H), 1.55 (d, J=6.4 Hz, 3H), 1.43-1.40 (m, 4H), 1.30 (s, 12H), 0.96 (s, 6H).

### Preparation of methyl 3-[[(S)-1-[2-(4,4-dimethyl-1-piperidyl)-3,6-dimethyl-4-oxo-chromen-8-yl]ethyl]amino]-2-(4,4,5,5-tetramethyl-1,3,2-dioxaborolan-2-yl)benzoate and methyl 3-[[(R)-1-[2-(4,4-dimethyl-1-piperidyl)-3,6-dimethyl-4-oxo-chromen-8-yl]ethyl]amino]-2-(4,4,5,5-tetramethyl-1,3,2-dioxaborolan-2-yl)benzoate

The residue was separated by SFC (column: DAICEL CHIRALPAKAD (250 mm×30 mm×10 um); mobile phase: [CO₂—IPA (0.1% NH₃H₂O)]; B %:24%, isocratic elution mode) to give the title compound, Isomer 1 (80 mg, 132.05 μmol, 38.86% yield) as light yellow oil and the title compound, Isomer 2 (70 mg, 116.75 μmol, 34.36% yield) as a white solid. Isomer 1: ¹H NMR (DMSO-d₆, 400 MHz) δ 7.64 (s, 1H), 7.39 (s, 1H), 7.18 (t, J=8.0 Hz, 1H), 7.02 (d, J=7.2 Hz, 1H), 6.55 (d, J=8.0 Hz, 1H), 5.21 (d, J=6.0 Hz, 1H), 5.04-5.01 (m, 1H), 3.78 (s, 3H), 3.38-3.35 (m, 4H), 2.32 (s, 3H), 1.91 (s, 3H), 1.55 (d, J=6.4 Hz, 3H), 1.44-1.41 (m, 4H), 1.30 (s, 12H), 0.96 (s, 6H). Isomer 2: ¹H NMR (DMSO-d₆, 400 MHz) δ 7.64 (s, 1H), 7.39 (s, 1H), 7.18 (t, J=8.0 Hz, 1H), 7.02 (d, J=7.2 Hz, 1H), 6.55 (d, J=8.0 Hz, 1H), 5.21 (d, J=6.4 Hz, 1H), 5.04-5.01 (m, 1H), 3.78 (s, 3H), 3.38-3.35 (m, 4H), 2.32 (s, 3H), 1.91 (s, 3H), 1.55 (d, J=6.4 Hz, 3H), 1.44-1.41 (m, 4H), 1.30 (s, 12H), 0.96 (s, 6H).

### Preparation of (S)-2-(4,4-dimethylpiperidin-1-yl)-8-(1-((1-hydroxy-3-oxo-1,3-dihydrobenzo[c][1,2]oxaborol-7-yl)amino)ethyl)-3,6-dimethyl-4H-chromen-4-one

To a solution of methyl 3-[[(1S)-1-[2-(4,4-dimethyl-1-piperidyl)-3,6-dimethyl-4-oxo-chromen-8-yl]ethyl]amino]-2-(4,4,5,5-tetramethyl-1,3,2-dioxaborolan-2-yl)benzoate (30 mg, 50.97 μmol, 1 eq) in THF (0.5 mL)/H₂O (0.3 mL) was added LiOH·H₂O (6.42 mg, 152.92 μmol, 3 eq) in portions at 0° C. Then the reaction mixture was warmed to 20° C. and stirred at 20° C. for 12 h. The reaction mixture was concentrated under reduced pressure and the residue was purified by prep-HPLC (column: Waters Xbridge Prep OBD C18 150×40 mm×10 um; mobile phase: [H₂O (10 mM NH₄HCO₃)-AC□]; gradient: 15%-45% B over 8.0 min) to give the title compound, Isomer 1 (22.0 mg, 44.77 μmol, 87.84% yield) as a white solid. ¹H NMR (MeOD, 400 MHz) δ 7.79 (s, 1H), 7.69 (s, 1H), 7.23 (d, J=7.2 Hz, 1H), 7.06 (t, J=7.6 Hz, 1H), 6.45 (d, J=8.0 Hz, 1H), 5.33-5.28 (m, 1H), 3.50-3.47 (m, 4H), 2.41 (s, 3H), 2.02 (s, 3H), 1.70 (d, J=6.8 Hz, 3H), 1.55-1.52 (m, 4H), 1.05 (s, 6H). MS (ESI): mass calcd. For C₂₇H₃₁BN₂O₅ 474.23 m/z found 475.2 [M+H]⁺. HPLC: 96.54% (220 nm), 97.21% (254 nm). Chiral purity: 100% ee.

### Preparation of (R)-2-(4,4-dimethylpiperidin-1-yl)-8-(1-((1-hydroxy-3-oxo-1,3-dihydrobenzo[c][1,2]oxaborol-7-yl)amino)ethyl)-3,6-dimethyl-4H-chromen-4-one

To a solution of methyl 3-[[(1R)-1-[2-(4,4-dimethyl-1-piperidyl)-3,6-dimethyl-4-oxo-chromen-8-yl]ethyl]amino]-2-(4,4,5,5-tetramethyl-1,3,2-dioxaborolan-2-yl)benzoate (50 mg, 84.96 μmol, 1 eq) in THF (0.5 mL)/H₂O (0.3 mL) was added LiOH·H₂O (11 mg, 254.87 μmol, 3 eq) in portions at 0° C., the reaction mixture was warmed to 20° C. and stirred at 20° C. for 12 h. The reaction mixture was concentrated under reduced pressure and the residue was purified by prep-HPLC (column: Waters Xbridge Prep OBD C18 150×40 mm×10 um; mobile phase: [H₂O (10 mM NH₄HCO₃)-AC□]; gradient: 15%-45% B over 8.0 min) to give the title compound, Isomer 2 (34.5 mg, 69.75 μmol, 82.10% yield) as a white solid. ¹H NMR (MeOD, 400 MHz) δ 7.80 (s, 1H), 7.69 (s, 1H), 7.24 (d, J=7.2 Hz, 1H), 7.06 (t, J=7.6 Hz, 1H), 6.46 (d, J=7.6 Hz, 1H), 5.33-5.31 (m, 1H), 3.49-3.47 (m, 4H), 2.41 (s, 3H), 2.02 (s, 3H), 1.70 (d, J=6.8 Hz, 3H), 1.55-1.52 (m, 4H), 1.05 (s, 6H). MS (ESI): mass calcd. For C₂₇H₃₁BN₂O₅ 474.23 m/z found 475.2 [M+H]⁺. HPLC: 95.90% (220 nm), 96.84% (254 nm). Chiral purity: 97.76% ee.

### Example 12

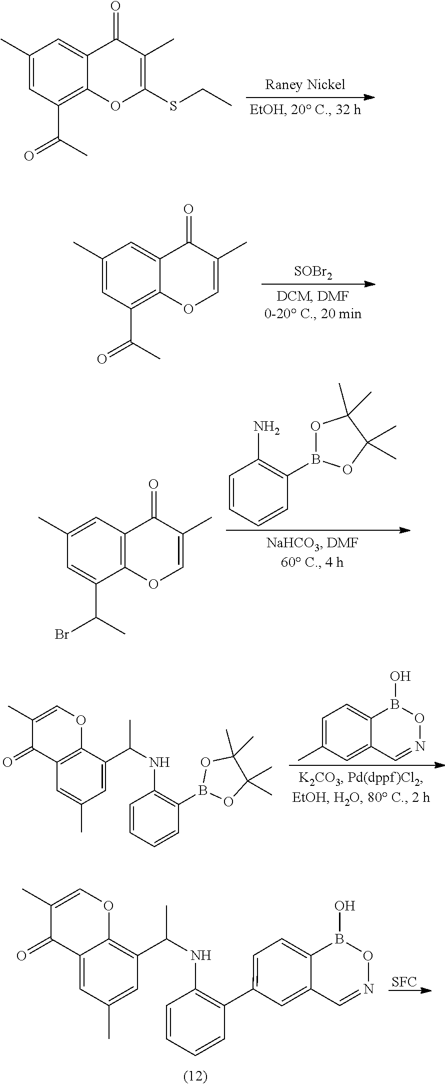

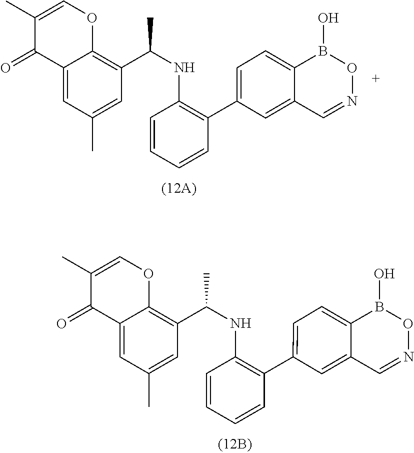 [SMILES: Cc1cc([C@@H](C)Nc2ccccc2-c2ccc3c(c2)C=NOB3O)c2occ(C)c(=O)c2c1 ; Cc1cc([C@H](C)Nc2ccccc2-c2ccc3c(c2)C=NOB3O)c2occ(C)c(=O)c2c1]

### Preparation of 8-(1-hydroxyethyl)-3,6-dimethyl-4H-chromen-4-one

To a solution of 8-acetyl-2-ethylsulfanyl-3,6-dimethyl-chromen-4-one (4.00 g, 14.47 mmol, 1 eq) in EtOH (100 mL) was added RANEY NICKEL (1.24 g, 14.47 mmol, 1 eq) under N₂, the suspension was degassed under vacuum and purged with N₂ several times. Then the resulting reaction mixture was stirred under N₂ at 20° C. for 32 h. The reaction mixture was filtered and the filter was concentrated in vacuo to give the title compound (4.00 g, crude) as a yellow solid, which was used directly for next step without further purification. ¹H NMR (CDCl₃, 400 MHz) δ 7.89 (s, 1H), 7.79 (s, 1H), 7.63 (s, 1H), 5.43-5.39 (m, 1H), 2.44 (s, 3H), 2.03 (s, 3H), 1.55 (d, J=6.4 Hz, 3H).

### Preparation of 8-(1-bromoethyl)-3,6-dimethyl-chromen-4-one

To a solution of 8-(1-hydroxyethyl)-3,6-dimethyl-chromen-4-one (1.00 g, 3.67 mmol, 1 eq) in DCM (10 mL)/DMF (2.68 mg, 36.7 μmol, 2.82 μL, 0.01 eq) was added dropwise thionylbromide (991 mg, 4.77 mmol, 369.2 μL, 1.3 eq) at 0° C., the reaction mixture was warmed to 20° C. and stirred at 20° C. for 20 min. The reaction mixture was poured into ice H₂O (10 mL) and extracted with DCM (10 mL×3). The combined organic layers were washed with sat.aq NaHCO₃ until pH=8, washed with brine (30 mL), dried over Na₂SO₄, filtered and concentrated under reduced pressure to give a residue. The residue was purified by flash silica gel chromatography (Biotage®; 12 g SepaFlash® Silica Flash Column, Eluent of 0~11% Ethyl acetate/Petroleum ether gradient@40 m/min) to give the title compound (700 mg, 2.49 mmol, 67.92% yield) as a yellow solid. ¹H NMR (CDCl₃, 400 MHz) δ 8.00 (s, 1H), 7.86 (s, 1H), 7.66 (d, J=2.0 Hz, 1H), 5.75 (q, J=6.8 Hz, 1H), 2.47 (s, 3H), 2.12 (d, J=6.8 Hz, 3H), 2.05 (s, 3H).

### Preparation of 3,6-dimethyl-8-[1-[2-(4,4,5,5-tetramethyl-1,3,2-dioxaborolan-2-yl) anilino]ethyl]chromen-4-one

To a solution of 8-(1-bromoethyl)-3,6-dimethyl-chromen-4-one (500 mg, 1.78 mmol, 1.00 eq), 2-(4,4,5,5-tetramethyl-1,3,2-dioxaborolan-2-yl)aniline (428.60 mg, 1.96 mmol, 1.10 eq) in DMF (5 mL) was added NaHCO₃ (149.40 mg, 1.78 mmol, 69.20 μL, 1.00 eq) in one portion at 20° C., the resulting reaction mixture was stirred at 60° C. for 4 h. After cooling to 25° C., the reaction mixture was diluted with water (20 mL) and extracted with MTBE (10 mL×3). The combined organic phase was washed with brine (10 mL), dried over Na₂SO₄, filtered and concentrated under reduced pressure. The crude product was purified by flash silica gel chromatography (Biotage®; 12 g SepaFlash® Silica Flash Column, Eluent of 0~23% Petroleum ether/Ethyl acetate gradient@40 mL/min) to give the title compound (580 mg, crude) as a white solid, which was used for directly for next step without further purification.

### Preparation of 8-[1-[2-(1-hydroxy-2,3,1-benzoxazaborinin-6-yl)anilino]ethyl]-3,6-dimethyl-chromen-4-one

A mixture of 6-bromo-1-hydroxy-2,3,1-benzoxazaborinine (250 mg, 1.11 mmol, 1.00 eq), 3,6-dimethyl-8-[1-[2-(4,4,5,5-tetramethyl-1,3,2-dioxaborolan-2-yl)anilino]ethyl]chromen-4-one (557 mg, 1.33 mmol, 1.20 eq), K₂CO₃ (306.00 mg, 2.21 mmol, 2 eq) and Pd(dppf)Cl₂ (81 mg, 110.70 μmol, 0.10 eq) in EtOH (2 mL)/H₂O (0.2 mL) was degassed and purged with N₂ for 3 times, and then the mixture was stirred at 80° C. for 2 h under N₂ atmosphere. The reaction mixture was cooled to 25° C., filtered through a pad of Celite and the filter cake was further washed with EtOH (10 mL). The filtrate was concentrated under reduced pressure and the crude product was further purified by reversed-phase HPLC (column: Waters Xbridge Prep OBD C18 150×40 mm×10 um; mobile phase: [H₂O (0.05% NH₃H₂O+10mMNH₄H CO₃)-AC□]; gradient: 10%-40% B over 8.0 min) to give the title compound (150 mg, 342.25 μmol, 30.92% yield) as a white solid. ¹H NMR (DMSO-d₆, 400 MHz) δ 9.34 (s, 1H), 8.67 (s, 1H), 8.17-8.15 (m, 2H), 7.84 (d, J=7.6 Hz, 1H), 7.80 (s, 1H), 7.70 (s, 1H), 7.56 (d, J=2.0 Hz, 1H), 7.08-7.04 (m, 2H), 6.69 (t, J=7.2 Hz, 1H), 6.51 (d, J=8.4 Hz, 1H), 5.12 (d, J=7.2 Hz, 1H), 5.00-4.93 (m, 1H), 2.35 (s, 3H), 1.91 (s, 3H), 1.40 (d, J=8.0 Hz, 3H). MS (ESI): mass calcd. For C₂₆H₂₃BN₂O₄ 438.18, m/z found 439.1 [M+H]⁺. HPLC: 98.67% (220 nm), 100.00% (254 nm).

### Preparation of 8-[(1S)-1-[2-(1-hydroxy-2,3,1-benzoxazaborinin-6-yl)anilino]ethyl]-3,6-dimethyl-chromen-4-one and 8-[(1R)-1-[2-(1-hydroxy-2,3,1-benzoxazaborinin-6-yl) anilino]ethyl]-3,6-dimethyl-chromen-4-one

8-[1-[2-(1-hydroxy-2,3,1-benzoxazaborinin-6-yl)anilino]ethyl]-3,6-dimethyl-chromen-4-one (0.18 g, 410.69 μmol, 1.00 eq) was separated by SFC (column: ChiralPak IH, 250×30 mm×10 um; mobile phase: [CO₂—IPA]; B %:32%, isocratic elution mode) to give the title compound, Isomer 1 (77.3 mg, 175.67 μmol, 42.77% yield) as a white solid and the title compound, Isomer 2 (78.6 mg, 177.74 mol, 43.28% yield) as a white solid. Isomer 1: ¹H NMR (DMSO-d₆, 400 MHz) δ 9.43 (s, 1H), 8.68 (s, 1H), 8.17-8.16 (m, 2H), 7.84 (d, J=7.6 Hz, 1H), 7.81 (s, 1H), 7.70 (s, 1H), 7.56 (d, J=2.0 Hz, 1H), 7.08-7.04 (m, 2H), 6.70 (t, J=7.2 Hz, 1H), 6.51 (d, J=8.0 Hz, 1H), 5.12 (d, J=7.2 Hz, 1H), 5.00-4.93 (m, 1H), 2.35 (s, 3H), 1.91 (s, 3H), 1.40 (d, J=6.8 Hz, 3H). MS (ESI): mass calcd. For C₂₆H₂₃BN₂O₄ 438.18, m/z found 439.1 [M+H]⁺. HPLC: 99.60% (220 nm), 98.68% (254 nm). Chiral purity: 100% ee.

Isomer 2: ¹H NMR (DMSO-d₆, 400 MHz) δ 9.43 (s, 1H), 8.68 (s, 1H), 8.17-8.16 (m, 2H), 7.84 (d, J=8.0 Hz, 1H), 7.81 (s, 1H), 7.70 (s, 1H), 7.56 (d, J=2.0 Hz, 1H), 7.07-7.04 (m, 2H), 6.70 (t, J=7.2 Hz, 1H), 6.51 (d, J=8.4 Hz, 1H), 5.12 (d, J=7.6 Hz, 1H), 5.00-4.93 (m, 1H), 2.35 (s, 3H), 1.91 (s, 3H), 1.40 (d, J=6.8 Hz, 3H). MS (ESI): mass calcd. For C₂₆H₂₃BN₂O₄ 438.18, m/z found 439.2 [M+H]⁺. HPLC: 99.11% (220 nm), 99.28% (254 nm). Chiral purity: 98.1% ee.

### Example 13

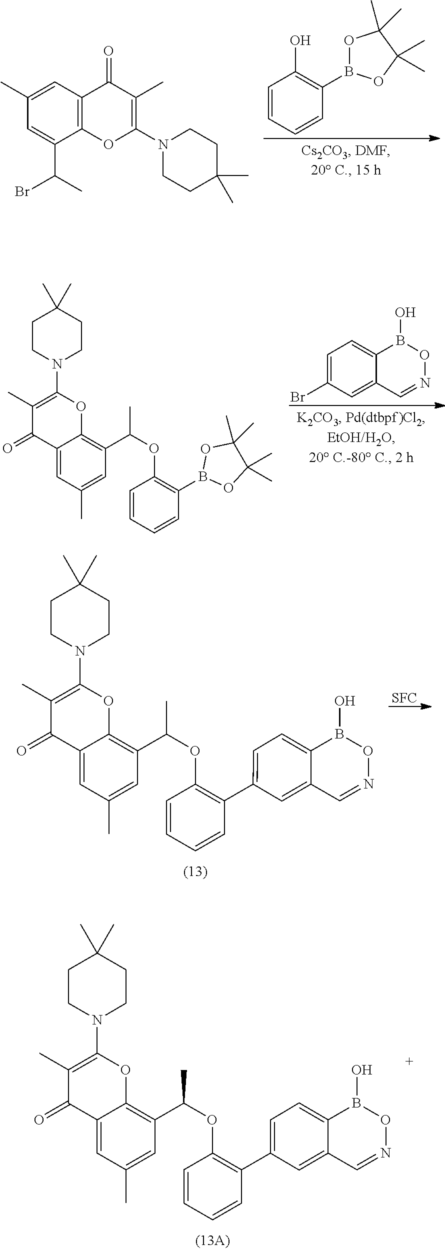

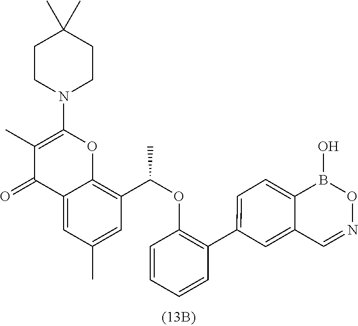 [SMILES: Cc1cc([C@H](C)Oc2ccccc2-c2ccc3c(c2)C=NOB3O)c2oc(N3CCC(C)(C)CC3)c(C)c(=O)c2c1]

### Preparation of 2-(4,4-dimethyl-1-piperidyl)-3,6-dimethyl-8-[1-[2-(4,4,5,5-tetramethyl-1,3,2-dioxaborolan-2-yl)phenoxy]ethyl]chromen-4-one

To a mixture of 8-(1-bromoethyl)-2-(4,4-dimethyl-1-piperidyl)-3,6-dimethyl-chromen-4-one (5.00 g, 12.7 mmol, 1 eq) and 2-(4,4,5,5-tetramethyl-1,3,2-dioxaborolan-2-yl)phenol (3.37 g, 15.3 mmol, 1.2 eq) in DMF (50 ml) was added Cs₂CO₃ (6.23 g, 19.12 mmol, 1.5 eq) in one portion at 20° C. under N₂, then the reaction mixture was stirred at 20° C. for 15 h. 3 parallel reactions were combined for work up.

The combined reaction mixture was poured into H₂O (1500 mL) at 0° C. and then extracted with MTBE (500 mL×3). The combined organic phase was washed with brine (600 mL×2), dried over Na₂SO₄, filtered and concentrated under reduced pressure to give the title compound (25.00 g, 47.04 mmol, 97.13% yield) as an off white solid used into the next step without further purification. ¹H NMR (DMSO-d₆, 400 MHz) δ 7.88 (s, 1H), 7.79 (d, J=2.0 Hz, 1H), 7.67 (dd, J=1.6, 6.0 Hz, 1H), 7.29-7.27 (m, 1H), 6.94 (t, J=7.2 Hz, 1H), 6.72 (d, J=8.4 Hz, 1H), 5.83 (q, J=6.4 Hz, 1H), 3.40-3.32 (m, 4H), 2.42 (s, 3H), 2.04 (s, 3H), 1.68 (d, J=6.0 Hz, 3H), 1.53-1.49 (m, 4H), 1.37 (s, 12H), 1.04 (s, 6H).

### Preparation of 2-(4,4-dimethyl-1-piperidyl)-8-[1-[2-(1-hydroxy-2,3,1-benzoxazaborinin-6-yl)phenoxy]ethyl]-3,6-dimethyl-chromen-4-one

To a mixture of 2-(4,4-dimethyl-1-piperidyl)-3,6-dimethyl-8-[1-[2-(4,4,5,5-tetramethyl-1,3,2-dioxaborolan-2-yl)phenoxy]ethyl]chromen-4-one (3.20 g, 4.82 mmol, 1 eq) and 6-bromo-1-hydroxy-2,3,1-benzoxazaborinine (1.20 g, 5.30 mmol, 1.1 eq) in EtOH (64 mL)/H₂O (8 mL) were added K₂CO₃ (1.33 g, 9.63 mmol, 2 eq) and ditert-butyl(cyclopentyl) phosphane;dichloropalladium;iron (314 mg, 482 mol, 0.1 eq) in turns at 20° C. under N₂, the reaction suspension was degassed under vacuum and purged with N₂ 3 times, then the reaction mixture was directly stirred at 80° C. (the oil bath was pre-heated to 80° C.) for 2 h under N₂. The reaction mixture was cooled to 25° C., filtered and concentrated in vacuo to remove EtOH. The residue was diluted with H₂O (100 mL), adjusted pH=7 with 2N HCl at 0° C. and the aqueous phase was extracted with EtOAc (80 mL×3). The combined organic layers were washed with brine (150 mL), dried over Na₂SO₄, filtered and concentrated under reduced pressure to give a residue. The residue was purified by prep-HPLC (column: Welch Utimate C18 250×70 mm×10 um; mobile phase: [H₂O (10 mM NH₄HCO₃)-AC□]; gradient: 50%-80% B over 0.1 min) to give the title compound (700 mg, 1.27 mmol, 28.07% yield) as a yellow solid. ¹H NMR (DMSO-d₆, 400 MHz) δ 9.40 (s, 1H), 8.59 (s, 1H), 8.06 (d, J=7.6 Hz, 1H), 7.88-7.83 (m, 2H), 7.64 (s, 1H), 7.40-7.33 (m, 3H), 7.11-7.07 (m, 2H), 5.83 (q, J=6.4 Hz, 1H), 3.24-3.22 (m, 4H), 2.27 (s, 3H), 1.87 (s, 3H), 1.63 (d, J=6.4 Hz, 3H), 1.36-1.28 (m, 4H), 0.92 (s, 6H). MS (ESI): mass calcd. For C₃₃H₃₅BN₂O₅ 550.26, m/z found 549.1 [M−H]⁻. HPLC: 99.46 (220 nm), 99.64 (240 nm).

### Preparation of 2-(4,4-dimethyl-1-piperidyl)-8-[(1R)-1-[2-(1-hydroxy-2,3,1-benzoxaza borinin-6-yl)phenoxy]ethyl]-3,6-dimethyl-chromen-4-one and 2-(4,4-dimethyl-1-piperidyl)-8-[(1S)-1-[2-(1-hydroxy-2,3,1-benzoxazaborinin-6-yl)phenoxy]ethyl]-3,6-dimethyl-chromen-4-one

The crude product (260 mg 90% purity) which was further separated by SFC (column: DAICEL CHIRALCEL OD (250 mm×30 mm×10 um); mobile phase: [CO₂—IPA]; B %:45%, isocratic elution mode) to give the title compound, Isomer 1 (35.0 mg, 62.88 μmol, 1.54% yield) as a white solid and the title compound, Isomer 2 (35.0 mg, 62.58 μmol, 1.54% yield) as a white solid. Isomer 1: ¹H NMR (DMSO-d₆, 400 MHz) δ 9.40 (s, 1H), 8.59 (s, 1H), 8.06 (d, J=7.6 Hz, 1H), 7.88-7.83 (m, 2H), 7.64 (s, 1H), 7.39 (dd, J=1.6, 6.0 Hz, 1H), 7.36 (d, J=2.4 Hz, 1H), 7.34-7.31 (m, 1H), 7.12-7.03 (m, 2H), 5.83 (q, J=6.4 Hz, 1H), 3.28-3.17 (m, 4H), 2.27 (s, 3H), 1.87 (s, 3H), 1.63 (d, J=6.4 Hz, 3H), 1.36-1.28 (m, 4H), 0.93 (s, 6H). MS (ESI): mass calcd. For C₃₃H₃₅BN₂O₅ 550.26, m/z found 551.3 [M+H]⁺. HPLC: 98.89 (220 nm), 99.72 (240 nm). Isomer 2: ¹H NMR (DMSO-d₆, 400 MHz) δ 9.40 (s, 1H), 8.59 (s, 1H), 8.06 (d, J=7.6 Hz, 1H), 7.89-7.81 (m, 2H), 7.64 (s, 1H), 7.39 (dd, J=1.6, 6.0 Hz, 1H), 7.36 (d, J=2.0 Hz, 1H), 7.34-7.30 (m, 1H), 7.13-7.03 (m, 2H), 5.83 (q, J=6.4 Hz, 1H), 3.27-3.18 (m, 4H), 2.27 (s, 3H), 1.87 (s, 3H), 1.63 (d, J=6.4 Hz, 3H), 1.35-1.29 (m, 4H), 0.92 (s, 6H). MS (ESI): mass calcd. For C₃₃H₃₅BN₂O₅ 550.26, m/z found 551.3 [M+H]⁺. HPLC: 98.42 (220 nm), 96.57 (240 nm).

### Example 14

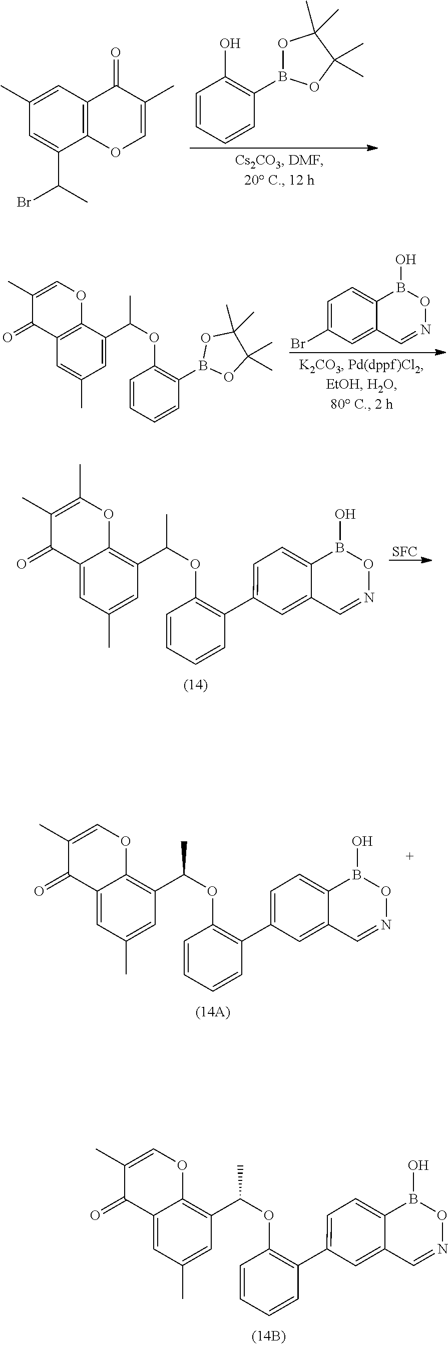

### Preparation of 3,6-dimethyl-8-[1-[2-(4,4,5,5-tetramethyl-1,3,2-dioxaborolan-2-yl)phenoxy]ethyl]chromen-4-one

To a solution of 8-(1-bromoethyl)-3,6-dimethyl-chromen-4-one (500 mg, 1.78 mmol, 1.00 eq), 2-(4,4,5,5-tetramethyl-1,3,2-dioxaborolan-2-yl)phenol (509 mg, 2.31 mmol, 1.30 eq) in DMF (5 mL) was added Cs₂CO₃ (927 mg, 2.85 mmol, 1.6 eq) in one portion at 20° C. The reaction mixture was stirred at 20° C. for 12 h. The reaction mixture was diluted with H₂O (20 mL) and extracted with MTBE (10 mL×3). The combined organic phases were washed with brine (10 mL), dried over Na₂SO₄, filtered and concentrated under reduced pressure. The crude product was purified by flash silica gel chromatography (Biotage®; 12 g SepaFlash® Silica Flash Column, Eluent of 0~23% Petroleum ether/Ethyl acetate gradient@40 mL/min) to give the title compound (450 mg, 1.07 mmol, 60.20% yield) as a yellow solid. ¹H NMR (DMSO-d₆, 400 MHz) δ 8.30 (s, 1H), 7.97 (s, 1H), 7.77 (s, 1H), 7.52 (dd, J=7.2 Hz, 1.6 Hz, 1H), 7.34-7.32 (m, 1H), 6.97 (d, J=8.4 Hz, 1H), 6.91 (t, J=7.4 Hz, 1H), 5.98 (q, J=6.0 Hz, 1H), 2.41 (s, 3H), 1.93 (s, 3H), 1.56 (d, J=6.0 Hz, 3H), 1.33 (s, 12H).

### Preparation of 8-[1-[2-(1-hydroxy-2,3,1-benzoxazaborinin-6-yl)phenoxy]ethyl]-3,6-dimethyl-chromen-4-one

A mixture of 6-bromo-1-hydroxy-2,3,1-benzoxazaborinine (180 mg, 797.04 μmol, 1.00 eq), 3,6-dimethyl-8-[1-[2-(4,4,5,5-tetramethyl-1,3,2-dioxaborolan-2-yl)phenoxy]ethyl]chromen-4-one (402 mg, 956.45 μmol, 1.20 eq), K₂CO₃ (221 mg, 1.59 mmol, 2.00 eq) and Pd(dppf)Cl₂ (59 mg, 79.70 μmol, 0.10 eq) in EtOH (2 mL)/H₂O (0.2 mL) was degassed and purged with N₂ for 3 times at 20° C., and then the mixture was stirred at 80° C. for 2 h under N₂ atmosphere. The reaction mixture was cooled to 25° C., filtered through a pad of Clite and the filter cake was washed with EtOH (10 mL). The filtrate was concentrated under reduced pressure and the crude product was further purified by reversed-phase HPLC (column: Waters Xbridge BEH C18 100×25 mm×10 um; mobile phase: [H₂O (10 mM NH₄HCO₃)-AC1]; gradient: 25%-55% B over 8.0 min) to give the title compound (180 mg, 405.14 μmol, 50.83% yield) as a white solid. ¹H NMR (DMSO-d₆, 400 MHz) δ 9.40 (s, 1H), 8.67 (s, 1H), 8.21 (s, 1H), 8.13 (d, J=8.0 Hz, 1H), 7.93-7.90 (m, 2H), 7.74-7.73 (m, 1H), 7.41-7.38 (m, 2H), 7.37-7.36 (m, 1H), 7.09-7.06 (m, 2H), 5.91-5.86 (m, 1H), 2.26 (s, 3H), 1.90 (s, 3H), 1.57 (d, J=6.4 Hz, 3H). MS (ESI): mass calcd. For C₂₆H₂₂BNO₅ 439.16, m/z found 440.2 [M+H]⁺. HPLC: 98.87% (220 nm), 99.26% (254 nm).

### Preparation of 8-[(1S)-1-[2-(1-hydroxy-2,3,1-benzoxazaborinin-6-yl)phenoxy]ethyl]-3,6-dimethyl-chromen-4-one and 8-[(1R)-1-[2-(1-hydroxy-2,3,1-benzoxazaborinin-6-yl) phenoxy]ethyl]-3,6-dimethyl-chromen-4-one

8-[1-[2-(1-hydroxy-2,3,1-benzoxazaborinin-6-yl)phenoxy]ethyl]-3,6-dimethyl-chromen-4-one (180 mg, 409.77 μmol, 1.00 eq) was separated by SFC (column: ChiralPak IH, 250×30 mm×10 um; mobile phase: [CO₂—IPA]; B %:29%, isocratic elution mode) to give the title compound, Isomer 1 (67.50 mg, 151.58 μmol, 36.99% yield) as a white solid and the title compound, Isomer 2 (77.80 mg, 177.11 mol, 43.22% yield) as a white solid. Isomer 1: ¹H NMR (DMSO-d₆, 400 MHz) δ 9.41 (s, 1H), 8.67 (s, 1H), 8.20 (d, J=1.2 Hz, 1H), 8.13 (d, J=8.0 Hz, 1H), 7.93-7.90 (m, 2H), 7.74-7.73 (m, 1H), 7.39-7.38 (m, 2H), 7.37-7.36 (m, 1H), 7.08-7.06 (m, 2H), 5.89 (q, J=6.4 Hz, 1H), 2.26 (s, 3H), 1.90 (s, 3H), 1.57 (d, J=6.0 Hz, 3H). MS (ESI): mass calcd. For C₂₆H₂₂BNO₅ 439.16, m/z found 440.1 [M+H]⁺. HPLC: 98.64% (220 nm), 99.41% (254 nm). Chiral purity: 97.86% ee. Isomer 2: ¹H NMR (DMSO-d₆, 400 MHz) δ 9.41 (s, 1H), 8.67 (s, 1H), 8.20 (d, J=1.2 Hz, 1H), 8.13 (d, J=8.0 Hz, 1H), 7.93-7.90 (m, 2H), 7.74-7.73 (m, 1H), 7.39-7.38 (m, 2H), 7.37-7.36 (m, 1H), 7.08-7.06 (m, 2H), 5.89 (q, J=6.4 Hz, 1H), 2.26 (s, 3H), 1.90 (s, 3H), 1.57 (d, J=6.0 Hz, 3H). MS (ESI): mass calcd. For C₂₆H₂₂BNO₅ 439.16, m/z found 440.1 [M+H]⁺. HPLC: 100.00% (220 nm), 100.00% (254 nm). Chiral purity: 98.9% ee.

### Example 16

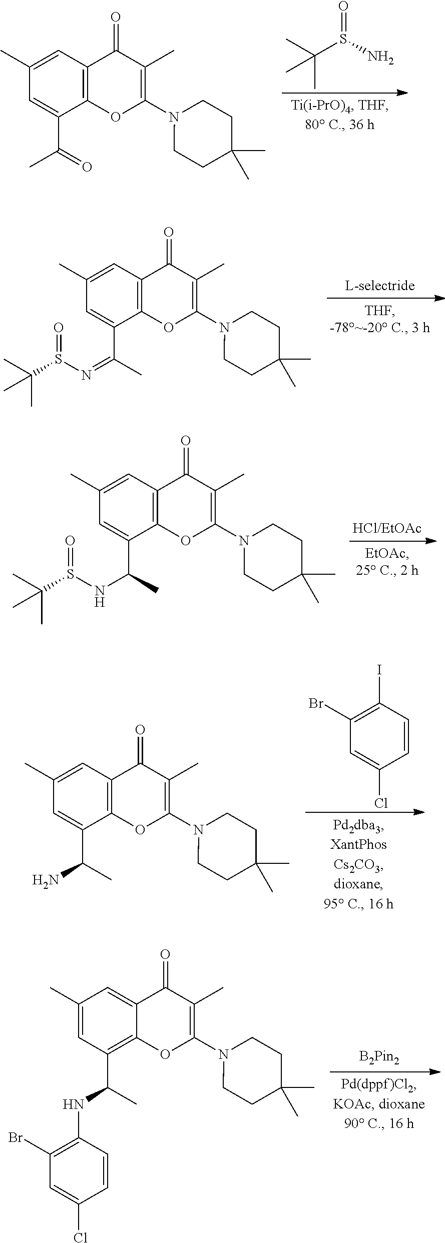

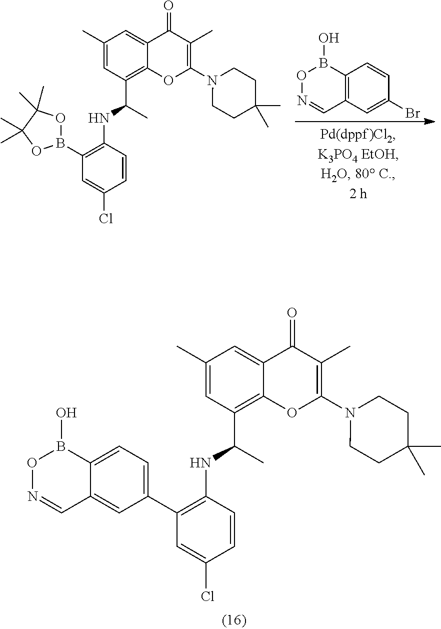

### Preparation of (NZ,S)—N-[1-[2-(4,4-dimethyl-1-piperidyl)-3,6-dimethyl-4-oxo-chromen-8-yl]ethylidene]-2-methyl-propane-2-sulfinamide

To a solution of -acetyl-2-(4,4-dimethylpiperidin-1-yl)-3,6-dimethyl-4H-chromen-4-one (50 g, 152.71 mmol and (S)-2-methylpropane-2-sulfinamide (37 g, 305.42 mmol, 2 eq) in THF (500 mL) was added Ti(i-PrO)₄ (196 g, 687.20 mmol, 202.8 mL, 4.5 eq) at 25° C. in one portion, the mixture was heated to 80° C. and stirred at 80° C. for 36 h. 3 parallel reactions were combined for work up. The reaction mixture was cooled to 20° C., poured into H₂O (2 L) and filtered. The filter cake was washed with EtOAc (500 mL×3) and the filtrate was extracted with EtOAc (2 L×3). The combined organic layers were washed with brine, dried over Na₂SO₄, filtered and concentrated under reduced pressure to give the residue. The residue was purified by column chromatography (SiO₂, Petroleum ether/Ethyl acetate=1:0 to 3:1) and then triturated with PE and EtOAc (PE:EtOAc=5:1, 1200 mL) to give the title compound (170 g, 394.80 mmol, 86.2% yield) as a light yellow solid. ¹H NMR (DMSO-d₆, 400 MHz) δ 7.85-7.76 (m, 1H), 7.55-7.39 (m, 1H), 3.36-3.34 (m, 4H), 2.76 (s, 2H), 2.54 (s, 1H), 2.41-2.40 (s, 3H), 1.91 (s, 3H), 1.43-1.41 (m, 4H), 1.21-1.09 (m, 9H), 0.98 (s, 6H).

### Preparation of (S)—N—((R)-1-(2-(4,4-dimethylpiperidin-J-yl)-3,6-dimethyl-4-oxo-4H-chromen-8-yl)ethyl)-2-methylpropane-2-sulfinamide

To a solution of (NZ,S)—N-[1-[2-(4,4-dimethyl-1-piperidyl)-3,6-dimethyl-4-oxo-chromen-8-yl]ethylidene]-2-methyl-propane-2-sulfinamide (56 g, 130.05 mmol, 1 eq) in THF (600 mL) was added L-selectride (1 M, 260.10 mL, 2 eq) dropwise at −78° C. under N₂, the mixture was stirred at −78° C. for 1 h. Then the reaction mixture was warmed to −20° C. and stirred at −20° C. for 2 h. 3 parallel reactions were combined for work up. The reaction mixture was warmed to 0° C. and quenched with sat.aq. NH₄Cl (500 mL) at 0° C. Then the reaction mixture was diluted with water (300 mL) and extracted with EtOAc (600 mL×3). The combined organic phases were washed with brine, dried over Na₂SO₄, filtered and concentrated under reduced pressure. The residue was further triturated with EtOAc (1000 mL) to give the title compound, Isomer 1 (94 g, 75.69 mmol, 55.7% yield) as a white solid. ¹H NMR (DMSO-d₆, 400 MHz) δ 7.65 (s, 1H), 7.53 (s, 1H), 5.50 (d, J=5.6 Hz, 1H), 4.95 (m, 1H), 3.41-3.39 (m, 4H), 2.37 (s, 3H), 1.90 (s, 3H), 1.55 (d, J=6.8 Hz, 3H), 1.48-1.46 (m, 4H), 1.08 (s, 9H), 1.00 (m, 6H).

### Preparation of (R)-8-(J-aminoethyl)-2-(4,4-dimethylpiperidin-1-yl)-3,6-dimethyl-4H-chromen-4-one

To a solution of (S)—N-[(1R)-1-[2-(4,4-dimethyl-1-piperidyl)-3,6-dimethyl-4-oxo-chromen-8-yl]ethyl]-2-methyl-propane-2-sulfinamide (42 g, 97.08 mmol, 1 eq) in EtOAc (300 mL) was added HCl/EtOAc (4 N, 300 mL) dropwise at 25° C., the mixture was stirred at 25° C. for 2 h. 2 reactions were combined for work up. The resulting suspension was directly filtered. Then the filter cake was dissolved in H₂O (400 mL), adjusted pH=12 with NH₃·H₂O and extracted with EtOAc (300 mL×3). The combined organic phase was washed with brine, dried over Na₂SO₄, filtered and concentrated under reduced pressure to give the title compound, Isomer 2 (50.7 g, 154.36 mmol, 79.5% yield) as a light yellow solid. ¹H NMR (DMSO-d₆, 400 MHz) δ 7.63 (s, 1H), 7.59 (s, 1H), 4.53-4.48 (m, 1H), 3.39-3.36 (m, 4H), 2.38 (s, 3H), 1.94-1.93 (br s, 2H), 1.89 (s, 3H), 1.47-1.45 (m, 4H), 1.31 (d, J=6.8 Hz, 3H), 1.00 (s, 6H).

### Preparation of (R)-8-(1-((2-bromo-4-chlorophenyl)amino)ethyl)-2-(4,4-dimethylpiperidin-1-yl)-3,6-dimethyl-4H-chromen-4-one

The title compound was prepared as a light yellow gum (330 mg, 637.21 μmol, 69.76% yield) according to the similar procedure in Example 78, using 8-[(1R)-1-aminoethyl]-2-(4,4-dimethyl-1-piperidyl)-3,6-dimethyl-chromen-4-one (300 mg, 913.39 μmol, 1 eq) and 2-bromo-4-chloro-1-iodo-benzene (580 mg, 1.83 mmol, 2 eq). ¹H NMR (CDCl₃, 400 MHz) δ 7.88 (s, 1H), 7.43 (d, J=8.4 Hz, 1H), 7.31 (s, 1H), 6.99 (d, J=8.8 Hz, 1H), 6.99 (d, J=8.8 Hz, 1H), 4.96-4.91 (m, 1H), 4.71-4.70 (m, 1H), 4.12 (q, J=7.2 Hz, 2H), 3.41-3.38 (m, 4H), 2.37 (s, 3H), 2.06 (s, 3H), 1.66 (d, J=6.8 Hz, 3H), 1.53-1.51 (m, 4H), 1.04 (s, 6H).

### Preparation of (R)-8-(1-((4-chloro-2-(4,4,5,5-tetramethyl-1,3,2-dioxaborolan-2-yl)phenyl)amino)ethyl)-2-(4,4-dimethylpiperidin-1-yl)-3,6-dimethyl-4H-chromen-4-one

The title compound was prepared as a yellow solid (350 mg, 619.52 μmol, 97.22% yield) according to the procedures described herein and the synthetic scheme, using 8-[(1R)-1-(2-bromo-4-chloro-anilino)ethyl]-2-(4,4-dimethyl-1-piperidyl)-3,6-dimethyl-chromen-4-one (330 mg, 637.21 μmol, 1 eq) and B₂Pin₂ (324 mg, 1.27 mmol, 2 eq). This product was used directly for next step without further purification.

### Preparation of (R)-8-(1-((4-chloro-2-(1-hydroxy-1H-benzo[d][1,2,6]oxazaborinin-6-yl)phenyl)amino)ethyl)-2-(4,4-dimethylpiperidin-1-yl)-3,6-dimethyl-4H-chromen-4-one

The title compound was prepared as (25.6 mg, 42.03 μmol, 15.82% yield) as a white solid according to the procedures described herein and the synthetic scheme, using 8-[(1R)-1-[4-chloro-2-(4,4,5,5-tetramethyl-1,3,2-dioxaborolan-2-yl)anilino]ethyl]-2-(4,4-dimethyl-1-piperidyl)-3,6-dimethyl-chromen-4-one (225 mg, 398.52 μmol, 1.5 eq) and 6-bromo-1-hydroxy-2,3,1-benzoxazaborinine (60 mg, 265.68 μmol, 1 eq). ¹H NMR (DMSO-d6, 400 MHz) δ 9.46 (br s, 1H), 8.65 (s, 1H), 8.13 (d, J=8.0 Hz, 1H), 7.87-7.84 (m, 2H), 7.60 (s, 1H), 7.44 (s, 1H), 7.15-7.12 (m, 1H), 7.08 (s, 1H), 6.53 (d, J=8.8 Hz, 1H), 5.25-5.24 (m, 1H), 4.91-4.88 (m, 1H), 3.32-3.24 (m, 4H), 2.32 (s, 3H), 1.89 (s, 3H), 1.46-1.44 (m, 3H), 1.40-1.37 (m, 4H), 0.95 (s, 6H). MS (ESI): mass calcd. For C₃₃H35BClN3O4 583.24, m/z found 584.3 [M+H]⁺. HPLC: 95.87% (220 nm), 96.33% (254 nm). Chiral purity: 98.02% ee.

### Example 17

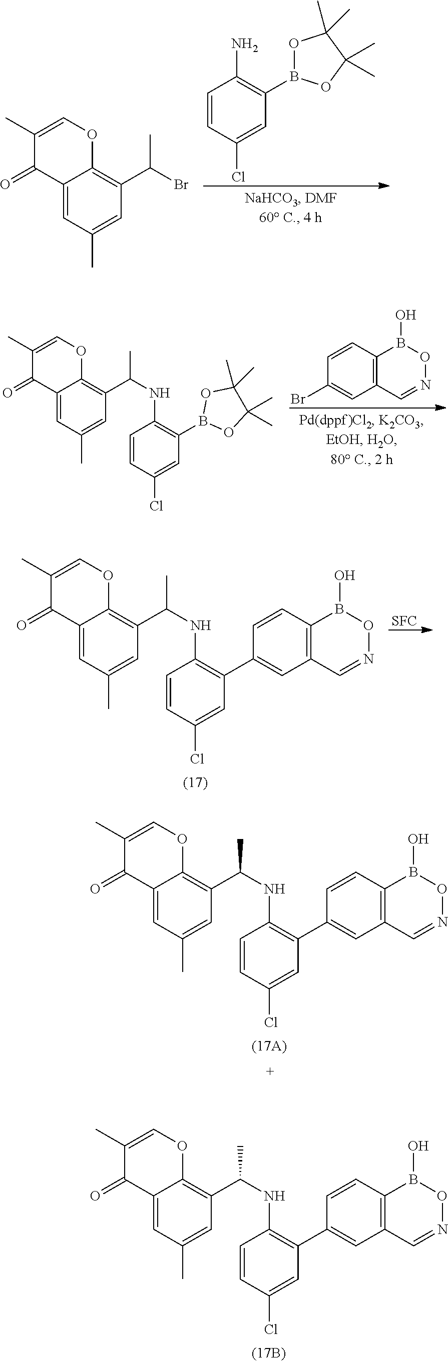

### Preparation of 8-[1-[4-chloro-2-(4,4,5,5-tetramethyl-1,3,2-dioxaborolan-2-yl)anilino]ethyl]-3,6-dimethyl-chromen-4-one

To a solution of 8-(1-bromoethyl)-3,6-dimethyl-chromen-4-one (500 mg, 1.78 mmol, 1.00 eq) and 4-chloro-2-(4,4,5,5-tetramethyl-1,3,2-dioxaborolan-2-yl)aniline (1.42 g, 1.96 mmol, 1.10 eq) in DMF (5 mL) was added NaHCO₃ (150 mg, 1.78 mmol, 69.20 μL, 1.00 eq) in portions at 20° C., the reaction mixture was heated to 60° C. and stirred at 60° C. for 4 h. The mixture was cooled to 25° C., diluted with water (20 mL), and extracted with MTBE (10 mL×3). The combined organic phases were washed with brine (10 mL), dried over Na₂SO₄, filtered and concentrated under reduced pressure. The crude product was purified by flash silica gel chromatography (Biotage®; 12 g SepaFlash® Silica Flash Column, Eluent of 0~23% Petroleum ether/Ethyl acetate gradient@40 mL/min) to give the title compound (400 mg, 881.51 μmol, 49.57% yield) as a yellow solid. ¹H NMR (DMSO-d₆, 400 MHz) δ 8.31 (s, 1H), 7.74 (s, 1H), 7.48 (d, J=2.0 Hz, 1H), 7.33 (d, J=2.8 Hz, 1H), 7.15 (dd, J=8.8, 2.8 Hz, 1H), 6.41 (d, J=7.2 Hz, 1H), 6.35 (d, J=9.2 Hz, 1H), 5.07-5.00 (m, 1H), 2.35 (s, 3H), 1.93 (s, 3H), 1.54 (d, J=6.8 Hz, 3H), 1.35 (s, 12H).

### Preparation of ethyl 8-[1-[4-chloro-2-(1-hydroxy-2,3,1-benzoxazaborinin-6-yl)anilino]ethyl]-3,6-dimethyl-chromen-4-one

A mixture of 6-bromo-1-hydroxy-2,3,1-benzoxazaborinine (150 mg, 664.20 μmol, 1.00 eq), 8-[1-[4-chloro-2-(4,4,5,5-tetramethyl-1,3,2-dioxaborolan-2-yl)anilino]ethyl]-3,6-dimethyl-chromen-4-one (332 mg, 730.62 μmol, 1.10 eq), Pd(dppf)Cl₂ (49 mg, 66.42 μmol, 0.10 eq) and K₂CO₃ (184 mg, 1.33 mmol, 2.00 eq) in EtOH (3 mL)/H₂O (0.3 mL) was degassed and purged with N₂ for 3 times at 20° C., and then the reaction mixture was heated to 80° C. and stirred at 80° C. for 2 h under N₂ atmosphere. The reaction mixture was cooled to 20° C. and filtered through a pad of Celite. The filter cake was washed with EtOH (10 mL) and the filtrate was concentrated under reduced pressure. The residue was purified by prep-HPLC (column: Phenomenex Gemini NX—C18(75×30 mm×3 um); mobile phase: [H₂O (10 mM NH₄HCO₃)-AC□]; gradient:25%-55% B over 8.0 min) to give the title compound (40 mg, 77.24 mol, 11.63% yield) as a yellow solid.

### Preparation of 8-[(1S)-1-[4-chloro-2-(1-hydroxy-2,3,1-benzoxazaborinin-6-yl)anilino]ethyl]-3,6-dimethyl-chromen-4-one and 8-[(R)-1-[4-chloro-2-(1-hydroxy-2,3,1-benzoxaza borinin-6-yl)anilino]ethyl]-3,6-dimethyl-chromen-4-one

8-[1-[4-chloro-2-(1-hydroxy-2,3,1-benzoxazaborinin-6-yl)anilino]ethyl]-3,6-dimethyl-chromen-4-one (40.00 mg, 84.62 μmol, 1.00 eq) was separated by SFC (column: ChiralPak IH, 250×30 mm, 10 um; mobile phase: [CO₂—IPA]; B %:25%, isocratic elution mode) to give the title compound, Isomer 1 (10.2 mg, 20.75 μmol, 24.53% yield) as an off-white solid and the title compound, Isomer 2 (11.3 mg, 22.85 μmol, 27.00% yield) as an off-white solid. Isomer 1: ¹H NMR (DMSO-d₆, 400 MHz) δ 9.47 (s, 1H), 8.68 (s, 1H), 8.18-8.16 (m, 2H), 7.86-7.83 (m, 2H), 7.71 (d, J=1.2 Hz, 1H), 7.53 (d, J=2.0 Hz, 1H), 7.11 (dd, J=8.4, 2.8 Hz, 1H), 7.08 (d, J=2.4 Hz, 1H), 6.49 (d, J=8.8 Hz, 1H), 5.31 (d, J=7.2 Hz, 1H), 4.97-4.90 (m, 1H), 2.35 (s, 3H), 1.91 (s, 3H), 1.42 (d, J=6.8 Hz, 3H). MS (ESI): mass calcd. For C₂₆H₂₂BClN₂O₄ 472.14, m/z found 473.2 [M+H]⁺. HPLC: 96.19% (220 nm), 96.79% (254 nm). Chiral purity: 97.98% ee. Isomer 2: ¹H NMR (DMSO-d₆, 400 MHz) δ 9.47 (s, 1H), 8.68 (s, 1H), 8.18-8.16 (m, 2H), 7.86-7.83 (m, 2H), 7.71 (d, J=1.6 Hz, 1H), 7.53 (d, J=2.0 Hz, 1H), 7.11 (dd, J=8.4, 2.8 Hz, 1H), 7.08 (d, J=2.4 Hz, 1H), 6.49 (d, J=8.8 Hz, 1H), 5.31 (d, J=7.2 Hz, 1H), 4.97-4.90 (m, 1H), 2.35 (s, 3H), 1.91 (s, 3H), 1.42 (d, J=6.8 Hz, 3H). MS (ESI): mass calcd. For C₂₆H₂₂BClN₂O₄ 472.14, m/z found 473.2 [M+H]⁺. HPLC: 95.58% (220 nm), 95.98% (254 nm). Chiral purity: 97.96% ee.

### Example 18

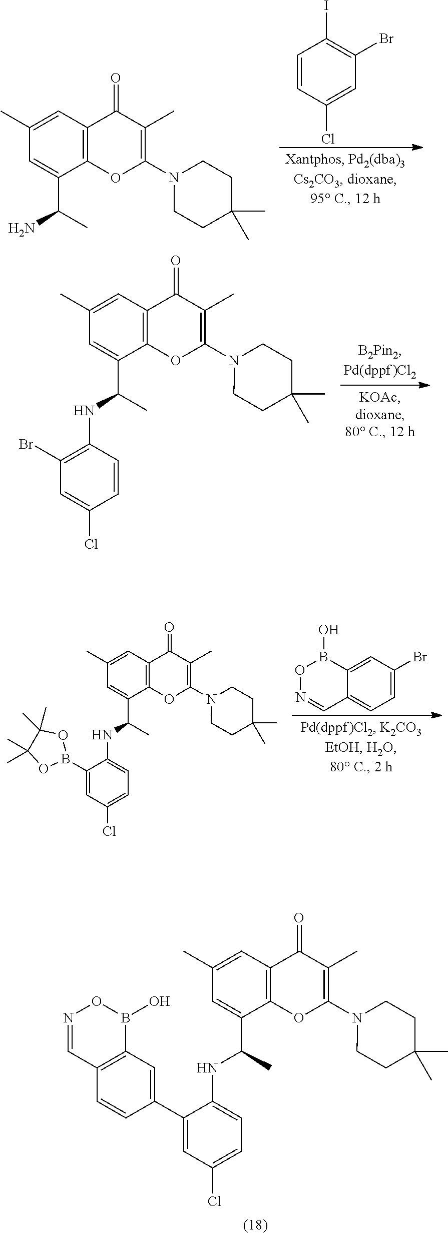

### Preparation of 8-[(1R)-1-(2-bromo-4-chloro-anilino)ethyl]-2-(4,4-dimethyl-1-piperidyl)-3,6-dimethyl-chromen-4-one

The title compound was prepared as light yellow oil (350 mg, crude) according to the similar procedure in Example 78, using 8-[(1R)-1-aminoethyl]-2-(4,4-dimethyl-1-piperidyl)-3,6-dimethyl-chromen-4-one (200 mg, 608.93 μmol, 1 eq) and 2-bromo-4-chloro-1-iodo-benzene (387 mg, 1.22 mmol, 2 eq). ¹H NMR (DMSO-d₆, 400 MHz) δ 7.63 (s, 1H), 7.53 (d, J=2.4 Hz, 1H), 7.42 (s, 1H), 7.10 (dd, J=8.8 Hz, J=2.4 Hz, 1H), 6.43 (d, J=8.8 Hz, 1H), 5.46 (d, J=6.8 Hz, 1H), 5.03-5.00 (m, 1H), 3.41-3.38 (m, 4H), 2.31 (s, 3H), 1.91 (s, 3H), 1.61 (d, J=6.8 Hz, 3H), 1.47-1.44 (m, 4H), 0.98 (s, 6H).

### Preparation of 8-[(1R)-1-[4-chloro-2-(4,4,5,5-tetramethyl-1,3,2-dioxaborolan-2-yl)anilino]ethyl]-2-(4,4-dimethyl-1-piperidyl)-3,6-dimethyl-chromen-4-one

The title compound was prepared as light yellow oil (270 mg, 477.92 μmol, 77.35% yield) according to the procedures described herein and the synthetic scheme, using 8-[(1R)-1-(2-bromo-4-chloro-anilino)ethyl]-2-(4,4-dimethyl-1-piperidyl)-3,6-dimethyl-chromen-4-one (320 mg, 617.90 μmol, 1 eq) and B₂Pin₂ (314 mg, 1.24 mmol, 2 eq). ¹H NMR (DMSO-d₆, 400 MHz) δ 7.93 (s, 1H), 7.64 (s, 1H), 7.35-7.33 (m, 2H), 7.17 (dd, J=8.8 Hz, J=2.8 Hz, 1H), 6.37 (d, J=2.4 Hz, 1H), 5.01-4.98 (m, 1H), 3.39-3.37 (m, 4H), 2.32 (s, 3H), 1.91 (s, 3H), 1.55 (d, J=6.8 Hz, 3H), 1.46-1.43 (m, 4H), 1.32 (s, 12H), 0.98 (s, 6H).

### Preparation of 8-[(1R)-1-[4-chloro-2-(1-hydroxy-2,3,1-benzoxazaborinin-7-yl)anilino]ethyl]-2-(4,4-dimethyl-1-piperidyl)-3,6-dimethyl-chromen-4-one

The title compound was prepared as white solid (29.4 mg, 49.10 μmol, 20.73% yield) according to the procedures described herein and the synthetic scheme, using 7-bromo-1-hydroxy-2,3,1-benzoxazaborinine (53.5 mg, 236.90 μmol, 1 eq) and 8-[(1R)-1-[4-chloro-2-(4,4,5,5-tetramethyl-1,3,2-dioxaborolan-2-yl)anilino]ethyl]-2-(4,4-dimethyl-1-piperidyl)-3,6-dimethyl-chromen-4-one (268 mg, 473.80 μmol, 2 eq). ¹H NMR (DMSO-d₆, 400 MHz) δ 9.42 (s, 1H), 8.70 (s, 1H), 8.16 (s, 1H), 7.95-7.93 (m, 1H), 7.84-7.82 (m, 1H), 7.60 (s, 1H), 7.43 (s, 1H), 7.14-7.12 (m, 1H), 7.07 (s, 1H), 6.51 (d, J=8.8 Hz, 1H), 5.22 (d, J=6.8 Hz, 1H), 4.93-4.89 (m, 1H), 3.30-3.28 (m, 4H), 2.32 (s, 3H), 1.90 (s, 3H), 1.44 (d, J=6.8 Hz, 3H), 1.41-1.38 (m, 4H), 0.96 (s, 6H). MS (ESI): mass calcd. For C₃₃H₃₅BClN₃O₄ 583.24 m/z found 584.1 [M+H]⁺. HPLC: 97.52% (220 nm), 98.281% (254 nm). Chiral purity: 100% ee.

### Example 19

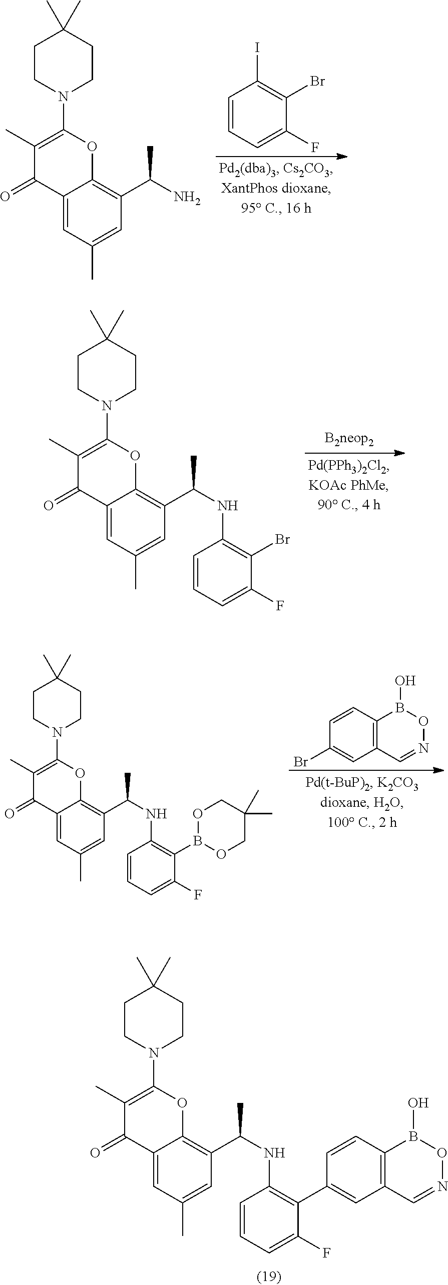

### Preparation of (R)-8-(1-((2-bromo-3-fluorophenyl)amino)ethyl)-2-(4,4-dimethylpiperidin-1-yl)-3,6-dimethyl-4H-chromen-4-one

The title compound was prepared as a yellow solid (300 mg, 538.46 μmol, 88.43% yield) according to the procedures described herein and the synthetic scheme, using (R)-8-(1-aminoethyl)-2-(4,4-dimethylpiperidin-1-yl)-3,6-dimethyl-4H-chromen-4-one (200 mg) and 2-bromo-1-fluoro-3-iodo-benzene (366 mg, 1.22 mmol, 2 eq). ¹H NMR (CDCl₃, 400 MHz) δ 7.88 (s, 1H), 7.35 (d, J=2.0 Hz, 1H), 6.97 (dd, J=14.4 Hz 8.0 Hz, 1H), 6.46 (t, J=7.6 Hz, 1H), 6.11 (d, J=8.4 Hz, 1H), 5.03-4.95 (m, 1H), 4.85-4.84 (m, 1H), 3.41-3.38 (m, 4H), 2.37 (s, 3H), 2.05 (s, 3H), 1.67 (d, J=6.8 Hz, 3H), 1.53-1.50 (m, 4H), 1.03 (s, 6H).

### Preparation of (R)-8-(1-((2-(5,5-dimethyl-1,3,2-dioxaborinan-2-yl)-3-fluorophenyl) amino)ethyl)-2-(4,4-dimethylpiperidin-1-yl)-3,6-dimethyl-4H-chromen-4-one

The title compound was prepared as a yellow solid (50 mg, 74.84 μmol, 75.05% yield) according to the procedures described herein and the synthetic scheme, using (R)-8-(1-((2-bromo-3-fluorophenyl)amino)ethyl)-2-(4,4-dimethylpiperidin-1-yl)-3,6-dimeth yl-4H-chromen-4-one (50 mg) and 2-(5,5-dimethyl-1,3,2-dioxaborinan-2-yl)-5,5-dimethyl-1,3,2-dioxaborinane (113 mg, 498.57 μmol, 5 eq). ¹H NMR (CDCl₃, 400 MHz) δ 7.87-7.86 (m, 1H), 7.40-7.39 (m, 1H), 7.04-6.98 (m, 1H), 6.32-6.28 (m, 1H), 6.01 (d, J=8.4 Hz, 1H), 5.00-4.95 (m, 1H), 3.68-3.66 (m, 4H), 3.40-3.37 (m, 4H), 2.36 (s, 3H), 2.06 (s, 3H), 1.57 (d, J=6.8 Hz, 3H), 1.52-1.49 (m, 4H), 1.03 (d, J=2.4 Hz, 6H), 0.99 (s, 6H).

### Preparation of (R)-2-(4,4-dimethylpiperidin-1-yl)-8-(1-((3-fluoro-2-(1-hydroxy-1H-benzo[d][1,2,6]oxazaborinin-6-yl)phenyl)amino)ethyl)-3,6-dimethyl-4H-chromen-4-one

The title compound was prepared as a white solid (16.7 mg, 28.97 μmol, 43.62% yield) according to the procedures described herein and the synthetic scheme, using (R)-8-(1-((2-(5,5-dimethyl-1,3,2-dioxaborinan-2-yl)-3-fluorophenyl)amino)ethyl)-2-(4,4-dimethylpiperidin-1-yl)-3,6-dimethyl-4H-chromen-4-one (46 mg). ¹H NMR (DMSO-d₆, 400 MHz) δ 8.50 (s, 1H), 8.07 (d, J=7.2 Hz, 1H), 7.64-7.61 (m, 3H), 7.38 (d, J=1.6 Hz, 1H), 7.13-7.07 (m, 1H), 6.53-6.49 (m, 1H), 6.35 (d, J=8.0 Hz, 1H), 6.07-6.00 (m, 2H), 4.99-4.92 (m, 1H), 4.87-4.86 (m, 1H), 3.13-3.07 (m, 4H), 2.32 (s, 3H), 1.90 (s, 3H), 1.43-1.41 (m, 7H), 0.96 (s, 6H). MS (ESI): mass calcd. For C₃₃H₃₅BFN₃₀₄ 567.27, m/z found 568.3 [M+H]⁺. HPLC: 98.45% (220 nm), 98.61% (254 nm).

### Example 20

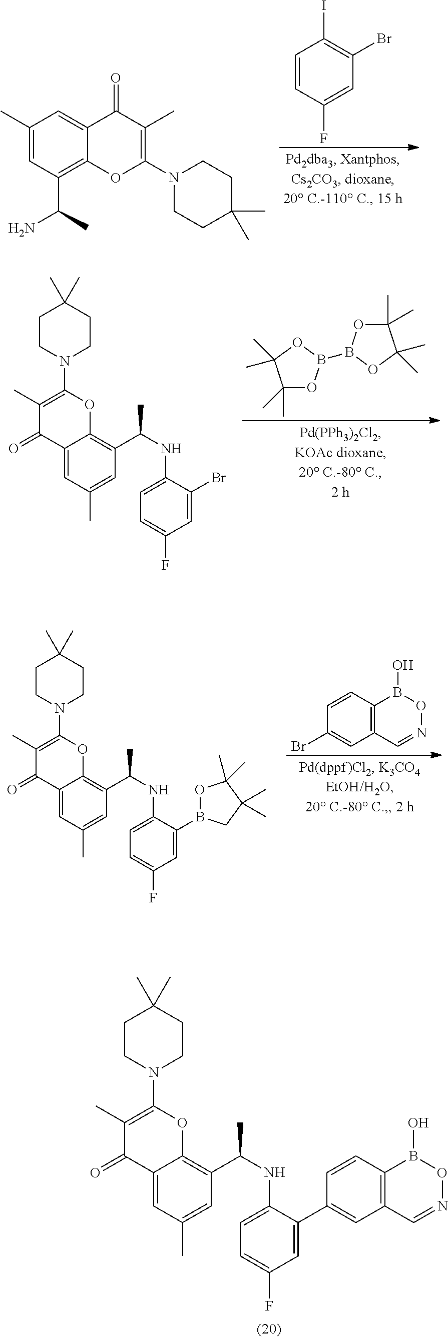

### Preparation of 8-[(1R)-1-(2-bromo-4-fluoro-anilino)ethyl]-2-(4,4-dimethyl-1-piperidyl)-3,6-dimethyl-chromen-4-one

The title compound was prepared as a yellow solid (225 mg, 448.72 μmol, 73.69% yield) according to the similar procedure in Example 78, using 8-[(1R)-1-aminoethyl]-2-(4,4-dimethyl-1-piperidyl)-3,6-dimethyl-chromen-4-one (200 mg, 609 μmol, 1 eq) and 2-bromo-4-fluoro-1-iodo-benzene (366 mg, 1.22 mmol, 2 eq). ¹H NMR (DMSO-d₆, 400 MHz) δ 7.63 (s, 1H), 7.43 (s, 1H), 7.41 (d, J=2.8 Hz, 1H), 6.98-6.93 (m, 1H), 6.44-6.40 (m, 1H), 5.03-4.96 (m, 1H), 3.41 (t, J=5.6 Hz, 4H), 2.31 (s, 3H), 1.91 (s, 3H), 1.61 (d, J=6.8 Hz, 3H), 1.46 (t, J=5.6 Hz, 4H), 0.99 (s, 6H).

### Preparation of 2-(4,4-dimethyl-1-piperidyl)-8-[(1R)-1-[4-fluoro-2-(4,4,5,5-tetramethyl-1,3,2-dioxaborolan-2-yl)anilino]ethyl]-3,6-dimethyl-chromen-4-one

The title compound was prepared as yellow oil (250 mg, 364.63 μmol, 91.42% yield) according to the procedures described herein and the synthetic scheme, using 8-[(1R)-1-(2-bromo-4-fluoro-anilino)ethyl]-2-(4,4-dimethyl-1-piperidyl)-3,6-dimethyl-chromen-4-one (200 mg, 399 μmol, 1 eq) and 4,4,5,5-tetramethyl-2-(4,4,5,5-tetramethyl-1,3,2-dioxaborolan-2-yl)-1,3,2-dioxaborolane (304 mg, 1.20 mmol, 3 eq). ¹H NMR (DMSO-d₆, 400 MHz) δ 7.93 (s, 1H), 7.63 (s, 1H), 7.36 (s, 1H), 7.13-7.10 (m, 1H), 7.02-6.99 (m, 1H), 6.33 (dd, J=9.2, 4.4 Hz, 1H), 4.98 (t, J=6.4 Hz, 1H), 3.38 (t, J=5.6 Hz, 4H), 2.32 (s, 3H), 1.91 (s, 3H), 1.54 (d, J=6.4 Hz, 3H), 1.45 (t, J=5.2 Hz, 4H), 1.07 (s, 12H), 0.98 (s, 6H).

### Preparation of 2-(4,4-dimethyl-1-piperidyl)-8-[(1R)-1-[4-fluoro-2-(1-hydroxy-2,3,1-benzoxazaborinin-6-yl)anilino]ethyl]-3,6-dimethyl-chromen-4-one

The title compound was prepared as an off-white solid (26.8 mg, 36.77 μmol, 12.61% yield) according to the procedures described herein and the synthetic scheme, using 2-(4,4-dimethyl-1-piperidyl)-8-[(1R)-1-[4-fluoro-2-(4,4,5,5-tetramethyl-1,3,2-dioxaborolan-2-yl)anilino]ethyl]-3,6-dimethyl-chromen-4-one (200.0 mg, 292 μmol, 1 eq) and 6-bromo-1-hydroxy-2,3,1-benzoxazaborinine (79.1 mg, 350 μmol, 1.2 eq). ¹H NMR (DMSO-d₆, 400 MHz) δ 9.47 (s, 1H), 8.64 (s, 1H), 8.14 (d, J=7.2 Hz, 1H), 7.89-7.84 (m, 2H), 7.60 (s, 1H), 7.46 (s, 1H), 6.96 (d, J=9.2 Hz, 2H), 6.54 (q, J=4.4 Hz, 1H), 5.04-5.02 (m, 1H), 4.85-4.82 (m, 1H), 3.30-3.22 (m, 4H), 2.32 (s, 3H), 1.89 (s, 3H), 1.43 (d, J=6.4 Hz, 3H), 1.38 (t, J=5.2 Hz, 4H), 0.95 (s, 6H). MS (ESI): mass calcd. For C₃₃H₃₅BFN₃O₄ 275.11, m/z found 276.2 [M+H]⁺. HPLC: 93.52 (220 nm), 94.91 (240 nm).

### Example 21

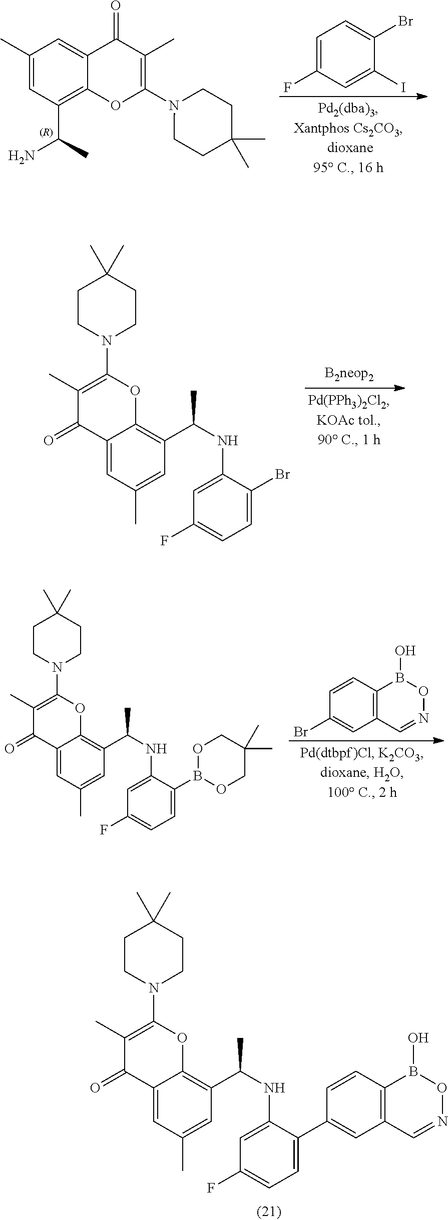

### Preparation of 8-[(1R)-1-(2-bromo-5-fluoro-anilino)ethyl]-2-(4,4-dimethyl-1-piperidyl)-3,6-dimethyl-chromen-4-one

The title compound was prepared as a yellow solid (280 mg, 558 μmol, 91.7% yield) according to the similar procedure in Example 78, using 1-bromo-4-fluoro-2-iodo-benzene (366 mg, 1.22 mmol, 2 eq) and 8-[(1R)-1-aminoethyl]-2-(4,4-dimethyl-1-piperidyl)-3,6-dimethyl-chromen-4-one (200 mg, 608 gmol, 1 eq). ¹H NMR (CDCl₃, 400 MHz) δ 7.90 (d, J=1.2 Hz, 1H), 7.39-7.33 (m, 2H), 6.29 (dt, J=2.8, 8.4 Hz, 1H), 6.09 (dd, J=2.8, 11.2 Hz, 1H), 4.97-4.91 (m, 1H), 4.82 (br d, J=5.2 Hz, 1H), 3.46-3.37 (m, 4H), 2.39 (s, 3H), 2.06 (s, 3H), 1.67 (d, J=6.8 Hz, 3H), 1.57-1.48 (m, 4H), 1.04 (s, 6H)

### Preparation of 8-[(1R)-1-[2-(5,5-dimethyl-1,3,2-dioxaborinan-2-yl)-5-fluoro-anilino]ethyl]-2-(4,4-dimethyl-1-piperidyl)-3,6-dimethyl-chromen-4-one

The title compound was prepared as a yellow solid (200 mg, 374.20 μmol, 93.82% yield) according to the procedures described herein and the synthetic scheme, using 8-[(1R)-1-(2-bromo-5-fluoro-anilino)ethyl]-2-(4,4-dimethyl-1-piperidyl)-3,6-dimethyl-chromen-4-one (200 mg, 398 μmol, 1 eq) and 2-(5,5-dimethyl-1,3,2-dioxaborinan-2-yl)-5,5-dimethyl-1,3,2-dioxaborinane (450 mg, 1.99 mmol, 5 eq). The product was used directly for next step without further purification.

### Preparation of 2-(4,4-dimethyl-1-piperidyl)-8-[(R)-1-[5-fluoro-2-(1-hydroxy-2,3,1-benzoxazaborinin-6-yl)anilino]ethyl]-3,6-dimethyl-chromen-4-one

The title compound was prepared as a a white solid (28.1 mg, 49.52 μmol, 26.47% yield) according to procedures described herein and the synthetic scheme, using 8-[(1R)-1-[2-(5,5-dimethyl-1,3,2-dioxaborinan-2-yl)-5-fluoro-anilino]ethyl]-2-(4,4-dimethyl-1-piperidyl)-3,6-dimethyl-chromen-4-one (150 mg, 280 μmol, 1.5 eq) and 6-bromo-1-hydroxy-2,3,1-benzoxazaborinine (42.2 mg, 187 μmol, 1 eq) and K₂CO₃ (51.7 mg, 374 μmol, 2 eq). ¹H NMR (DMSO-d₆, 400 MHz) δ 9.51-9.34 (m, 1H), 8.64 (s, 1H), 8.13 (d, J=8.0 Hz, 1H), 7.87-7.73 (m, 2H), 7.62 (d, J=1.6 Hz, 1H), 7.48 (d, J=1.6 Hz, 1H), 7.12-6.96 (m, 1H), 6.49 (dt, J=2.4, 8.4 Hz, 1H), 6.33 (dd, J=2.4, 12.0 Hz, 1H), 5.37 (br d, J=7.2 Hz, 1H), 5.02-4.89 (m, 1H), 3.42-3.33 (m, 2H), 3.29-3.22 (m, 2H), 2.33 (s, 3H), 1.90 (s, 3H), 1.52-1.45 (m, 3H), 1.44-1.31 (m, 4H), 0.95 (s, 6H). MS (ESI): mass calcd. For C₃₃H₃₅BFN₃O₄ 567.27, m/z found 568.3 [M+H]⁺. HPLC: 96.65% (220 nm), 95.61% (254 nm). Chiral purity: 96.3% ee.

### Example 22

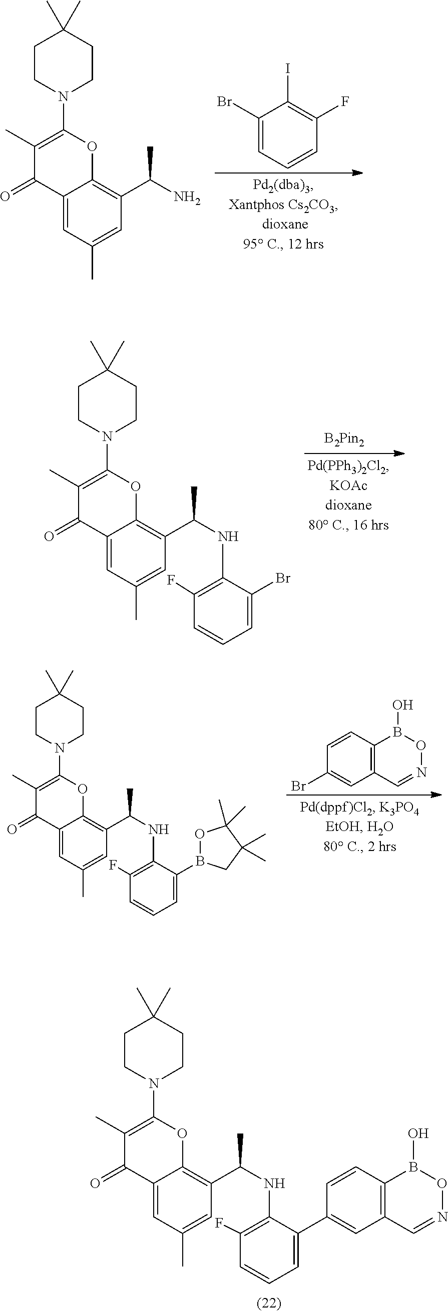

### Preparation of 8-[(1R)-1-(2-bromo-6-fluoro-anilino)ethyl]-2-(4,4-dimethyl-1-piperidyl)-3,6-dimethyl-chromen-4-one

The title compound was prepared as light yellow oil (0.32 g, 638.18 μmol, 83.84% yield) according to the similar procedure in Example 78, using 8-[(1R)-1-aminoethyl]-2-(4,4-dimethyl-1-piperidyl)-3,6-dimethyl-chromen-4-one (250 mg, 761.16 μmol, 1 eq) and 1-bromo-3-fluoro-2-iodo-benzene (458.06 mg, 1.52 mmol, 2 eq). ¹H NMR (CDCl₃, 400 MHz) δ 7.84 (s, 1H), 7.31 (s, 1H), 7.22 (d, J=8.0 Hz, 1H), 6.85-6.82 (m, 1H), 6.61-6.58 (m, 1H), 5.49-5.46 (m, 1H), 4.43-4.40 (m, 1H), 3.41-3.38 (m, 4H), 2.37 (s, 3H), 2.04 (s, 3H), 1.62 (s, J=6.8 Hz, 3H), 1.54-1.52 (m, 4H), 1.05 (s, 6H).

### Preparation of 2-(4,4-dimethyl-1-piperidyl)-8-[(1R)-1-[2-fluoro-6-(4,4,5,5-tetramethyl-1,3,2-dioxaborolan-2-yl)anilino]ethyl]-3,6-dimethyl-chromen-4-one

The title compound was prepared as light yellow oil (340 mg, crude) according to the procedure described in preceding examples, using 8-[(1R)-1-(2-bromo-6-fluoro-anilino)ethyl]-2-(4,4-dimethyl-1-piperidyl)-3,6-dimethyl-chromen-4-one (300 mg, 598.29 μmol, 1 eq) and B₂Pin₂ (379.82 mg, 1.50 mmol, 2.5 eq).

The product was used directly for next step without further purification. ¹H NMR (CDCl₃, 400 MHz) δ 7.84 (s, 1H), 7.44-7.41 (m, 2H), 6.99-6.93 (m, 1H), 6.66-6.62 (m, 1H), 5.96-5.94 (m, 1H), 5.54-5.50 (m, 1H), 3.39-3.36 (m, 4H), 2.38 (s, 3H), 2.04 (s, 3H), 1.55 (d, J=6.4 Hz, 3H), 1.53-1.50 (m, 4H), 1.35 (d, J=8.0 Hz, 12H), 1.04 (s, 6H).

### Preparation of 2-(4,4-dimethyl-1-piperidyl)-8-[(1R)-1-[2-fluoro-6-(1-hydroxy-2,3,1-benzoxazaborinin-6-yl)anilino]ethyl]-3,6-dimethyl-chromen-4-one

The title compound was prepared as white solid (49.3 mg, 84.49 μmol, 14.48% yield) according to the procedures described herein and the synthetic scheme, using 2-(4,4-dimethyl-1-piperidyl)-8-[(1R)-1-[2-fluoro-6-(4,4,5,5-tetramethyl-1,3,2-dioxaborolan-2-yl)anilino]ethyl]-3,6-dimethyl-chromen-4-one (320 mg, 583.42 μmol, 1 eq) and 6-bromo-1-hydroxy-2,3,1-benzoxazaborinine (119 mg, 525.07 mol, 0.9 eq). ¹H NMR (DMSO-d₆, 400 MHz) δ 9.30 (s, 1H), 8.31 (s, 1H), 7.91 (d, J=7.6 Hz, 1H), 7.67 (d, J=8.0 Hz, 1H), 7.57 (s, 1H), 7.50 (s, 1H), 7.41 (s, 1H), 7.23-7.21 (m, 1H), 6.93-6.86 (m, 2H), 5.17-5.13 (m, 1H), 4.47-4.43 (m, 1H), 3.04-3.00 (m, 4H), 2.30 (s, 3H), 1.80 (s, 3H), 1.34-1.28 (m, 7H), 0.95 (s, 6H). MS (ESI): mass calcd. For C₃₃H₃₅BFN₃O₄ 567.27, m/z found 568.1 [M+H]⁺. HPLC: 97.25% (220 nm), 97.32% (254 nm). Chiral purity: 96.72% ee.

### Example 23

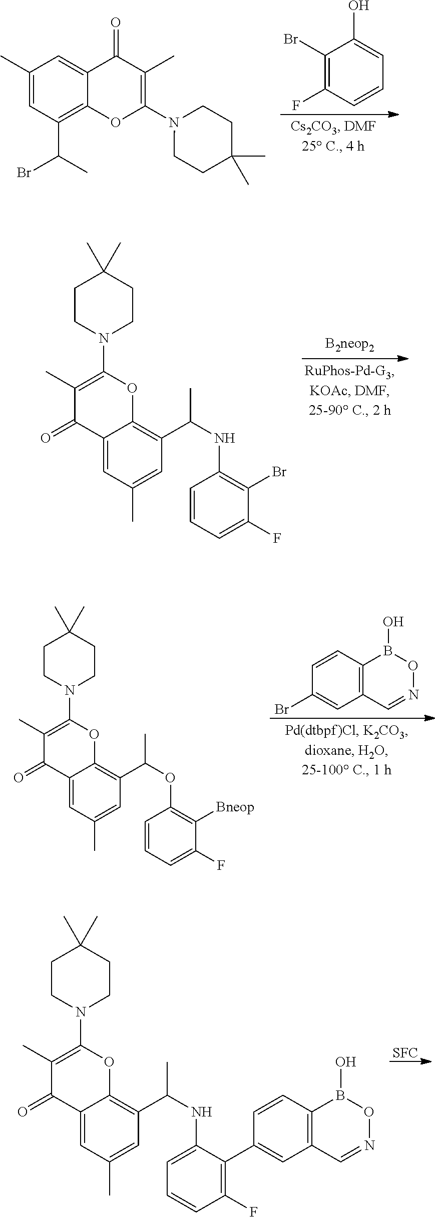

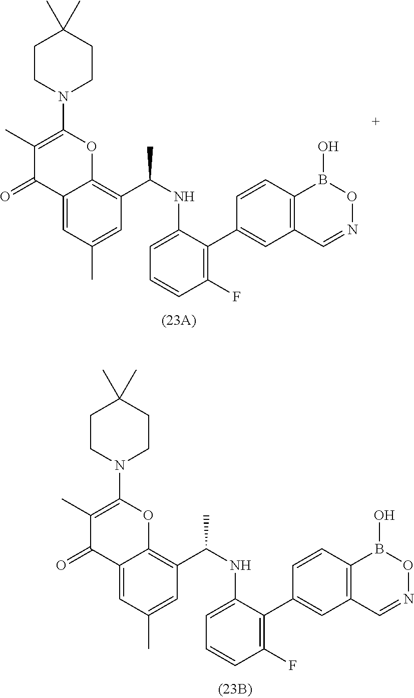 [SMILES: Cc1cc([C@@H](C)Nc2cccc(F)c2-c2ccc3c(c2)C=NOB3O)c2oc(N3CCC(C)(C)CC3)c(C)c(=O)c2c1 ; Cc1cc([C@H](C)Nc2cccc(F)c2-c2ccc3c(c2)C=NOB3O)c2oc(N3CCC(C)(C)CC3)c(C)c(=O)c2c1]

### Preparation of 8-(1-(2-bromo-3-fluorophenoxy)ethyl)-2-(4,4-dimethylpiperidin-1-yl)-3,6-dimethyl-4H-chromen-4-one

A mixture of 8-(1-bromoethyl)-2-(4,4-dimethyl-1-piperidyl)-3,6-dimethyl-chromen-4-one (1.00 g, 2.55 mmol, 1.0 eq), 2-bromo-3-fluoro-phenol (535 mg, 2.80 mmol, 1.1 eq) and Cs₂CO₃ (1.33 g, 4.08 mmol, 1.6 eq) in DMF (15 mL) was degassed and purged with N₂ for 3 times at 25° C., and then the reaction mixture was stirred at 25° C. for 4 h under N₂ atmosphere. The reaction mixture was quenched by adding water (30 mL) and then extracted with EtOAc (30 mL×3). The combined organic layers were washed with brine (20 mL×2), dried over Na₂SO₄, filtered and concentrated under reduced pressure to give a residue. The residue was purified by flash silica gel chromatography (Biotage®; 12 g Sepa Flash® Silica Flash Column, Eluent of 0~30% Ethyl acetate/Petroleum ether gradient@40 mL/min) to give the title compound (900 mg, 1.79 mmol, 70.28% yield) as an off white solid. ¹H NMR (DMSO-d₆, 400 MHz) δ 7.70 (s, 1H), 7.52 (s, 1H), 7.29 (q, J=6.8 Hz, 1H), 6.94 (t, J=6.8 Hz, 1H), 6.87 (d, J=8.8 Hz, 1H), 5.98 (t, J=6.4 Hz, 1H), 3.35-3.33 (m, 4H), 2.36 (s, 3H), 1.90 (s, 3H), 1.43-1.40 (m, 4H), 0.97 (s, 6H).

### 1.2.2 Preparation of 8-[1-[2-(5,5-dimethyl-1,3,2-dioxaborinan-2-yl)-3-fluoro-phenoxy]ethyl]-2-(4,4-dimethyl-1-piperidyl)-3,6-dimethyl-chromen-4-one

The title compound was prepared as an off white solid (300 mg, 560 μmol, 93.83% yield) according to the procedures described herein and the synthetic scheme, using 8-[1-(2-bromo-3-fluoro-phenoxy)ethyl]-2-(4,4-dimethyl-1-piperidyl)-3,6-dimethyl-chromen-4-one (300 mg, 600 μmol, 1 eq) and 2-(5,5-dimethyl-1,3,2-dioxaborinan-2-yl)-5,5-dimethyl-1,3,2-dioxaborinane (270 mg, 1200 μmol, 2 eq).

¹H NMR (DMSO-d₆, 400 MHz) δ 7.67 (s, 1H), 7.53 (s, 1H), 7.24-7.21 (m, 1H), 6.70-6.64 (m, 1H), 5.90-5.88 (m, 1H), 4.32 (t, J=5.2 Hz, 1H), 3.10 (s, 4H), 3.14-3.13 (m, 4H), 1.91 (s, 1H), 1.65 (d, J=6.4 Hz, 3H), 1.41 (t, J=5.2 Hz, 4H), 0.95 (d, J=7.2 Hz, 4H).

### Preparation of 2-(4,4-dimethyl-1-piperidyl)-8-[1-[3-fluoro-2-(1-hydroxy-2,3,1-benzoxazaborinin-6-yl)phenoxy]ethyl]-3,6-dimethyl-chromen-4-one

The title compound was prepared as a white solid (60 mg, 105 μmol, 21.19% yield) according to the procedures described herein and the synthetic scheme, using 6-bromo-1-hydroxy-2,3,1-benzoxazaborinine (112 mg, 498.02 μmol, 1 eq) and 8-[1-[2-(5,5-dimethyl-1,3,2-dioxaborinan-2-yl)-3-fluoro-phenoxy]ethyl]-2-(4,4-dimethyl-1-piperidyl)-3,6-dimethyl-chromen-4-one (400 mg, 747 μmol, 1.5 eq). ¹H NMR (DMSO-d₆, 400 MHz) δ 9.42 (s, 1H), 8.59 (s, 1H), 8.08 (d, J=7.6 Hz, 1H), 7.72-7.70 (m, 2H), 7.65 (s, 1H), 7.39-7.37 (m, 1H), 7.24 (s, 1H), 6.98 (t, J=7.6 Hz, 2H), 5.84 (d, J=6.4 Hz, 1H), 3.26-3.22 (m, 4H), 2.26 (s, 3H), 1.88 (s, 3H), 1.59 (d, J=6.0 Hz, 3H), 1.35-1.31 (m, 4H), 0.94 (s, 6H). MS (ESI): mass calcd. For C₃₃H₃₄BFN₂O₅ 568.25, m/z found 569.2 [M+H]⁺. HPLC: 97.61% (220 nm), 97.66% (254 nm).

### Preparation of 2-(4,4-dimethyl-1-piperidyl)-8-[(S)-1-[3-fluoro-2-(1-hydroxy-2,3,1-benzoxa zaborinin-6-yl)phenoxy]ethyl]-3,6-dimethyl-chromen-4-one and 2-(4,4-dimethyl-1-piperidyl)-8-[(1R)-1-[3-fluoro-2-(1-hydroxy-2,3,1-benzoxazaborinin-6-yl)phenoxy]ethyl]-3,6-dimethyl-chromen-4-one

2-(4,4-dimethyl-1-piperidyl)-8-[1-[3-fluoro-2-(1-hydroxy-2,3,1-benzoxazaborinin-6-yl)phenoxy]ethyl]-3,6-dimethyl-chromen-4-one (60 mg, 97.61% purity) was separated by SFC (column: ChiralPak IH, 250×30 mm, 10 um; mobile phase: [CO₂-EtOH]; B %: 25%, isocratic elution mode) to give the title compound, Isomer 1 (23 mg, 40 μmol, 38.15% yield) as a white solid and the title compound, Isomer 2 (23 mg, 40 μmol, 37.67% yield) as a white solid. Isomer 1: ¹H NMR (DMSO-d₆, 400 MHz) δ 9.45 (s, 1H), 8.60 (s, 1H), 8.08 (d, J=8.0 Hz, 1H), 7.73-7.70 (m, 2H), 7.65 (s, 1H), 7.39-7.35 (m, 1H), 7.24 (s, 1H), 6.98 (q, J=8.4 Hz, 2H), 5.84 (q, J=6.4 Hz, 1H), 3.27-3.21 (m, 4H), 2.26 (s, 3H), 1.88 (s, 3H), 1.59 (d, J=6.4 Hz, 3H), 1.35-1.31 (m, 4H), 0.93 (s, 6H). MS (ESI): mass calcd. For C₃₃H₃₄BFN₂O₅ 568.25, m/z found 569.2 [M+H]⁺. HPLC: 98.27% (220 nm), 98.15% (254 nm). Chiral purity: 96.36% ee. Isomer 2: ¹H NMR (DMSO-d₆, 400 MHz) δ 9.45 (s, 1H), 8.60 (s, 1H), 8.08 (d, J=8.0 Hz, 1H), 7.73-7.70 (m, 2H), 7.64 (s, 1H), 7.39-7.35 (m, 1H), 7.24 (s, 1H), 6.97 (q, J=8.4 Hz, 2H), 5.84 (q, J=6.4 Hz, 1H), 3.27-3.21 (m, 4H), 2.26 (s, 3H), 1.88 (s, 3H), 1.59 (d, J=6.4 Hz, 3H), 1.35-1.31 (m, 4H), 0.93 (s, 6H). MS (ESI): mass calcd. For C₃₃H₃₄BFN₂O₅ 568.25, m/z found 569.2 [M+H]⁺. HPLC: 99.52% (220 nm), 100.00% (254 nm). Chiral purity: 98.96% ee.

### Example 24

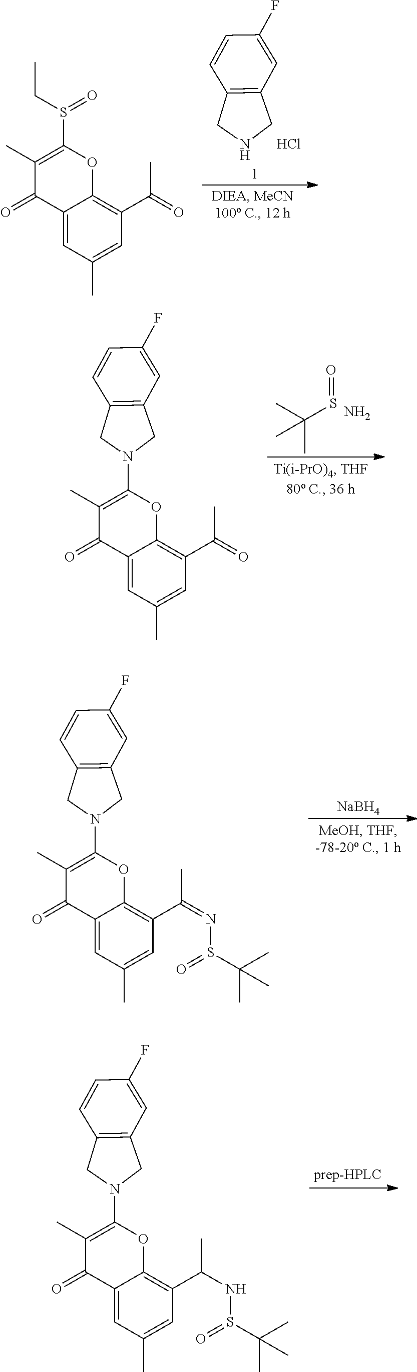

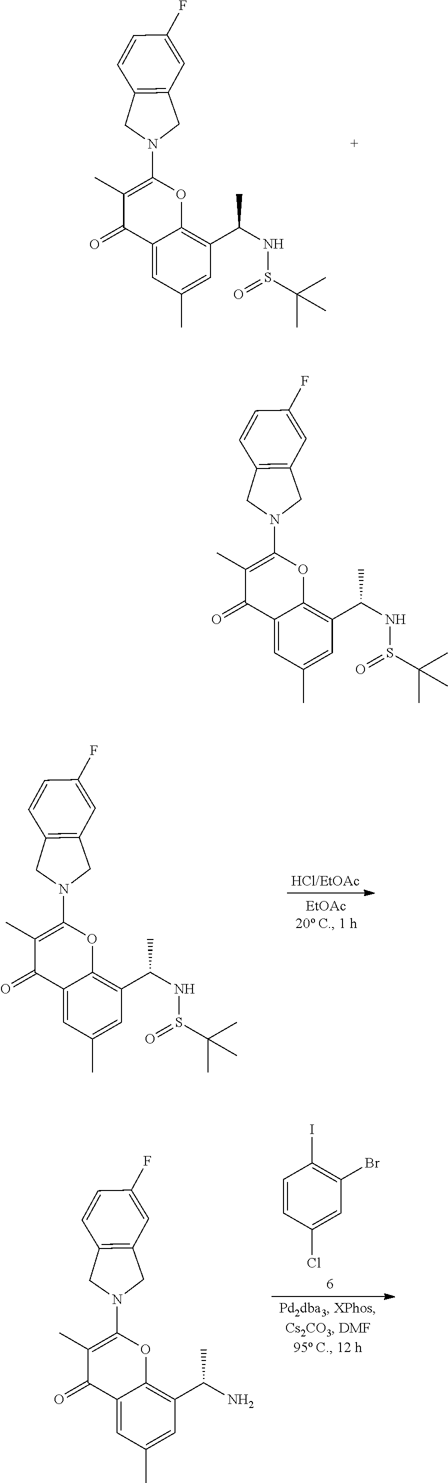 [SMILES: Cc1cc([C@@H](C)NS(=O)C(C)(C)C)c2oc(N3Cc4ccc(F)cc4C3)c(C)c(=O)c2c1 ; Cc1cc([C@H](C)NS(=O)C(C)(C)C)c2oc(N3Cc4ccc(F)cc4C3)c(C)c(=O)c2c1 ; Cc1cc([C@H](C)NS(=O)C(C)(C)C)c2oc(N3Cc4ccc(F)cc4C3)c(C)c(=O)c2c1 ; Cc1cc([C@H](C)N)c2oc(N3Cc4ccc(F)cc4C3)c(C)c(=O)c2c1 ; Clc1ccc(I)c(Br)c1]

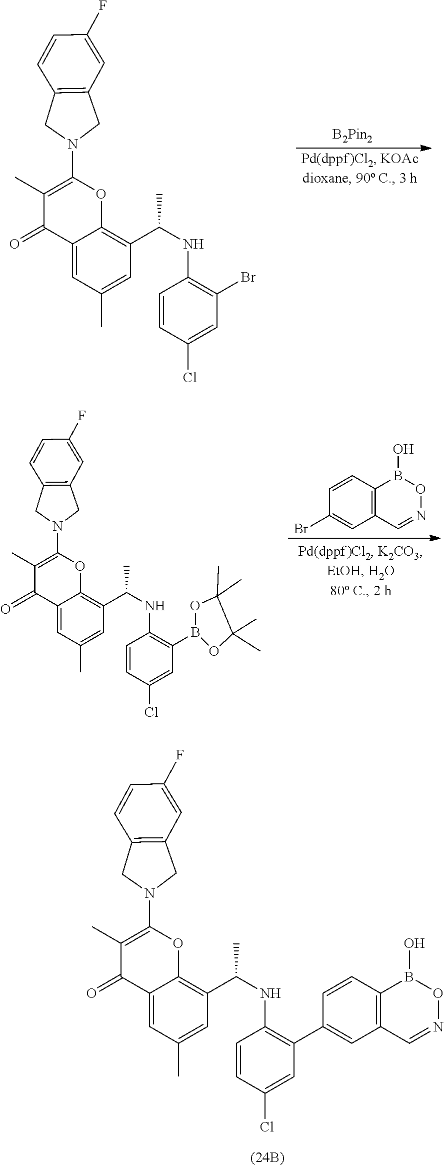 [SMILES: Cc1cc([C@H](C)Nc2ccc(Cl)cc2Br)c2oc(N3Cc4ccc(F)cc4C3)c(C)c(=O)c2c1 ; Cc1cc([C@H](C)Nc2ccc(Cl)cc2B2OC(C)(C)C(C)(C)O2)c2oc(N3Cc4ccc(F)cc4C3)c(C)c(=O)c2c1 ; OB1ON=Cc2cc(Br)ccc21 ; Cc1cc([C@H](C)Nc2ccc(Cl)cc2-c2ccc3c(c2)C=NOB3O)c2oc(N3Cc4ccc(F)cc4C3)c(C)c(=O)c2c1]

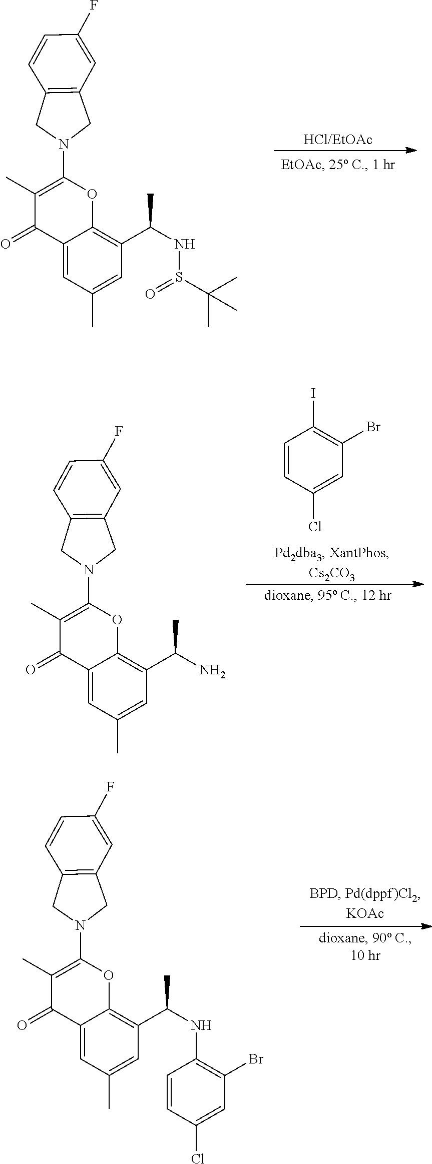

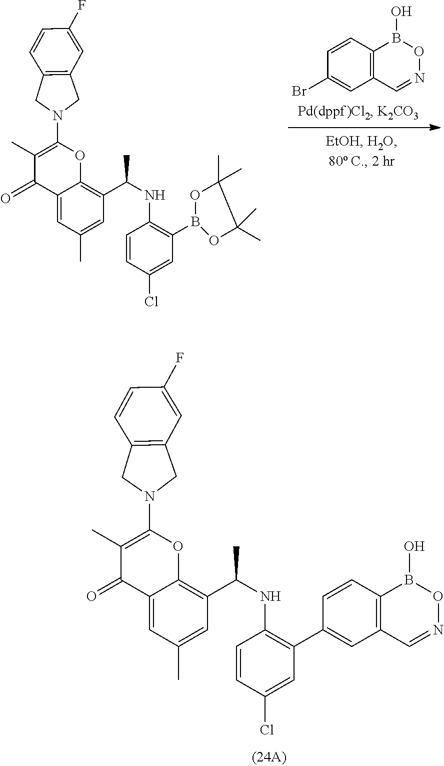 [SMILES: Cc1cc([C@@H](C)Nc2ccc(Cl)cc2B2OC(C)(C)C(C)(C)O2)c2oc(N3Cc4ccc(F)cc4C3)c(C)c(=O)c2c1 ; OB1ON=Cc2cc(Br)ccc21 ; Cc1cc([C@@H](C)Nc2ccc(Cl)cc2-c2ccc3c(c2)C=NOB3O)c2oc(N3Cc4ccc(F)cc4C3)c(C)c(=O)c2c1]

### Preparation of 8-acetyl-2-(5-fluoroisoindolin-2-yl)-3,6-dimethyl-chromen-4-one

To a solution of 8-acetyl-2-ethylsulfinyl-3,6-dimethyl-chromen-4-one (4.00 g, 13.68 mmol, 1.00 eq) and 5-fluoroisoindoline (2.61 g, 15.05 mmol, 1.10 eq, HCl) in MeCN (40 mL) was added DIEA (5.31 g, 41.05 mmol, 7.15 mL, 3.00 eq) dropwise at 20° C., the reaction mixture was heated to 100° C. and stirred at 100° C. for 12 h. The reaction mixture was cooled to 25° C. and concentrated under reduced pressure. The crude product was purified by flash silica gel chromatography (Biotage®; 40 g SepaFlash® Silica Flash Column, Eluent of 0~87% Petroleum ether/Ethyl acetate gradient@40 mL/min) to give 8-acetyl-2-(5-fluoroisoindolin-2-yl)-3,6-dimethyl-chromen-4-one (2.8 g, 7.97 mmol, 58.24% yield) as a brown solid. ¹H NMR (DMSO-d₆, 400 MHz) δ 7.96 (s, 1H), 7.87 (s, 1H), 7.46-7.43 (m, 1H), 7.30 (d, J=8.8 Hz, 1H), 7.19-7.15 (m, 1H), 5.15-5.12 (m, 4H), 2.73 (s, 3H), 2.42 (s, 3H), 2.22 (s, 3H).

### Preparation of (NZ,S)—N-[1-[2-(5-fluoroisoindolin-2-yl)-3,6-dimethyl-4-oxo-chromen-8-yl]ethylidene]-2-methyl-propane-2-sulfinamide

To a solution of 8-acetyl-2-(5-fluoroisoindolin-2-yl)-3,6-dimethyl-chromen-4-one (2.80 g, 7.97 mmol, 1.00 eq) and (S)-2-methylpropane-2-sulfinamide (1.93 g, 15.94 mmol, 2.00 eq) in THF (28 mL) was added Ti(i-PrO)₄ (9.06 g, 31.88 mmol, 9.41 mL, 4.00 eq) dropwise at 20° C. The reaction mixture was heated to 80° C. and stirred at 80° C. for 36 h. The reaction mixture was cooled to 20° C., diluted with water (60 mL) and then filtered. The filter cake was washed with EtOAc (25 mL×3) and the filtrate was extracted with EtOAc (25 mL×3). The combined organic layers were washed with brine (25 mL), dried over Na₂SO₄, filtered and concentrated under reduced pressure. The crude product was purified by flash silica gel chromatography (Biotage®; 40 g SepaFlash® Silica Flash Column, Eluent of 0~70% Petroleum ether/Ethyl acetate gradient@80 mL/min) to give (NZ,S)—N-[1-[2-(5-fluoroisoindolin-2-yl)-3,6-dimethyl-4-oxo-chromen-8-yl]ethylidene]-2-methyl-propane-2-sulfinamide (3.20 g, 7.04 mmol, 88.34% yield) as a brown solid. ¹H NMR (DMSO-d₆, 400 MHz) δ 7.87-7.77 (m, 1H), 7.58-7.38 (m, 2H), 7.29-7.24 (m, 1H), 7.19-7.17 (m, 1H), 5.20-5.02 (m, 4H), 2.80-2.60 (m, 3H), 2.42-2.40 (m, 3H), 2.24-2.22 (m, 3H), 1.09-1.07 (s, 9H).

### Preparation of (S)—N-[(1R)-1-[2-(5-fluoroisoindolin-2-yl)-3,6-dimethyl-4-oxo-chromen-8-yl]ethyl]-2-methyl-propane-2-sulfinamide and (S)—N-[(1S)-1-[2-(5-fluoroisoindolin-2-yl)-3,6-dimethyl-4-oxo-chromen-8-yl]ethyl]-2-methyl-propane-2-sulfinamide

To a solution of (NZ,S)—N-[1-[2-(5-fluoroisoindolin-2-yl)-3,6-dimethyl-4-oxo-chromen-8-yl]ethylidene]-2-methyl-propane-2-sulfinamide (3.20 g, 7.04 mmol, 1.00 eq) in EtOH (16 mL)/THF (16 mL) was added NaBH₄ (0.40 g, 10.57 mmol, 1.50 eq) in portions at −78° C., the reaction mixture was warmed to 20° C. and stirred at 20° C. for 1 h. The mixture was poured into ice water (50 mL) in portions at 0° C. and extracted with DCM (20 mL×3). The combined organic phase was washed with brine (20 mL), dried over Na₂SO₄, filtered and concentrated under reduced pressure. The crude product was purified by prep-HPLC (column: Agela DuraShell C18 250×70 mm×10 um; mobile phase: [H₂O (10 mM NH₄HCO₃)-AC□]; gradient:37%-58% B over 17.0 min) to give the title compound, Isomer 1 (0.49 g, 1.06 mmol, 15.04% yield) as a white solid and the title compound, isomer 2 (1.70 g, 3.48 mmol, 49.41% yield) as a white solid. Isomer 1: ¹H NMR (DMSO-d₆, 400 MHz) δ 7.66 (s, 1H), 7.52 (s, 1H), 7.47-7.47 (m, 1H), 7.31 (d, J=8.0 Hz, 1H), 7.20-7.16 (m, 1H), 5.54 (d, J=4.8 Hz, 1H), 5.24-5.11 (m, 4H), 5.03 (q, J=6.8 Hz, 1H), 2.37 (s, 3H), 2.22 (s, 3H), 1.60 (d, J=7.2 Hz, 3H), 1.07 (s, 9H). Isomer 2: ¹H NMR (DMSO-d₆, 400 MHz) δ 7.66 (s, 1H), 7.58 (s, 1H), 7.45-7.43 (m, 1H), 7.29 (d, J=8.8 Hz, 1H), 7.18-7.15 (m, 1H), 5.80 (d, J=7.2 Hz, 1H), 5.23-5.13 (m, 4H), 4.99 (q, J=6.8 Hz, 1H), 2.39 (s, 3H), 2.22 (s, 3H), 1.55 (d, J=6.8 Hz, 3H), 1.09 (s, 9H).

### Preparation of 8-[(1S)-1-aminoethyl]-2-(5-fluoroisoindolin-2-yl)-3,6-dimethyl-chromen-4-one

To a solution of (S)—N-[(1S)-1-[2-(5-fluoroisoindolin-2-yl)-3,6-dimethyl-4-oxo-chromen-8-yl]ethyl]-2-methyl-propane-2-sulfinamide (1.70 g, 3.72 mmol, 1.00 eq) in EtOAc (0.5 mL) was added HCl/EtOAc (4 N, 25.50 mL) dropwise at 20° C. The reaction mixture was stirred at 20° C. for 1 h. The resulting suspension was directly filtered, the filter cake was dissolved in water (10 mL), adjusted pH=9 with concentrated NH₃·H₂O and filtered. The filter cake was dried in vacuo to give 8-[(1S)-1-aminoethyl]-2-(5-fluoroisoindolin-2-yl)-3,6-dimethyl-chromen-4-one (1.00 g, 2.70 mmol, 72.63% yield) as a white solid. ¹H NMR (DMSO-d₆, 400 MHz) δ 7.67 (s, 1H), 7.64 (s, 1H), 7.47-7.44 (m, 1H), 7.29 (d, J=8.4 Hz, 1H), 7.21-7.18 (m, 1H), 5.22-5.13 (m, 4H), 4.74 (q, J=6.0 Hz, 1H), 2.40 (s, 3H), 2.23 (s, 3H), 1.48 (d, J=6.8 Hz, 3H).

### Preparation of 8-[(1S)-1-(2-bromo-4-chloro-anilino)ethyl-2-(5-fluoroisoindolin-2-yl)-3,6-dimethyl-chromen-4-one

The title compound was prepared as a yellow solid (360 mg, 664.40 μmol, 78.05% yield) according to the procedures described herein and the synthetic scheme, using 8-[(1S)-1-aminoethyl]-2-(5-fluoroisoindolin-2-yl)-3,6-dimethyl-chromen-4-one (300 mg, 851.30 μmol, 1.00 eq) and 2-bromo-4-chloro-1-iodo-benzene (405 mg, 1.28 mmol, 1.50 eq). ¹H NMR (DMSO-d₆, 400 MHz) δ 7.64-7.62 (m, 1H), 7.44-7.42 (m, 1H), 7.38-7.37 (m, 1H), 7.30-7.26 (m, 2H), 7.17-7.16 (m, 1H), 7.03 (d, J=8.8 Hz, 1H), 6.52-6.48 (m, 2H), 5.22-5.10 (m, 5H), 2.31 (s, 3H), 2.25 (s, 3H), 1.54 (d, J=2.8 Hz, 3H).

### Preparation of 8-[(1S)-1-[4-chloro-2-(4,4,5,5-tetramethyl-1,3,2-dioxaborolan-2-yl) anilino]ethyl]-2-(5-fluoroisoindolin-2-yl)-3,6-dimethyl-chromen-4-one

The title compound was prepared as a yellow solid (260 mg, 441.50 μmol, 66.45% yield) according to the procedures described herein and the synthetic scheme, using 8-[(1S)-1-(2-bromo-4-chloro-anilino)ethyl]-2-(5-fluoroisoindolin-2-yl)-3,6-dimethyl-chromen-4-one (360 mg, 664.40 μmol, 1.00 eq) and B₂Pin₂ (675 mg, 2.66 mmol, 4.00 eq). ¹H NMR (DMSO-d₆, 400 MHz) δ 7.64 (s, 1H), 7.44-7.42 (m, 1H), 7.34 (d, J=2.8 Hz, 1H), 7.31 (d, J=2.0 Hz, 1H), 7.28-7.26 (m, 1H), 7.19-7.16 (m, 2H), 6.39-6.36 (m, 2H), 5.19-5.10 (m, 5H), 2.31 (s, 3H), 2.25 (s, 3H), 1.59 (d, J=6.4 Hz, 3H), 1.33 (s, 12H).

### Preparation of 8-[(1S)-1-[4-chloro-2-(1-hydroxy-2,3,1-benzoxazaborinin-6-yl)anilino]ethyl]-2-(5-fluoroisoindolin-2-yl)-3,6-dimethyl-chromen-4-one

The title compound was prepared as a white solid (22.40 mg, 36.19 μmol, 8.20% yield) according to the procedures described herein and the synthetic scheme, using 8-[(1S)-1-[4-chloro-2-(4,4,5,5-tetramethyl-1,3,2-dioxaborolan-2-yl)anilino]ethyl]-2-(5-fluoroisoindolin-2-yl)-3,6-dimethyl-chromen-4-one (260 mg, 441.50 μmol, 1.00 eq) and 6-bromo-1-hydroxy-2,3,1-benzoxazaborinine (200 mg, 883.00 μmol, 2.00 eq). ¹H NMR (DMSO-d₆, 400 MHz) δ 9.40 (s, 1H), 8.61 (s, 1H), 8.11 (d, J=7.6 Hz, 1H), 7.82-7.81 (m, 2H), 7.62 (d, J=1.2 Hz, 1H), 7.40-7.39 (m, 2H), 7.25-7.23 (m, 1H), 7.16-7.13 (m, 2H), 7.07 (d, J=2.8 Hz, 1H), 6.58 (d, J=9.2 Hz, 1H), 5.33-5.31 (m, 1H), 5.13-4.97 (m, 5H), 2.32 (s, 3H), 2.24 (s, 3H), 1.48 (d, J=6.8 Hz, 3H). MS (ESI): mass calcd. For C₃₄H₂₈BClFN₃O₄ 607.18, m/z found 608.3 [M+H]⁺. HPLC: 98.21% (220 nm), 99.51% (254 nm). Chiral purity: 100% ee.

### Preparation of methyl (R)-8-(1-aminoethyl)-2-(5-fluoroisoindolin-2-yl)-3,6-dimethyl-4H-chromen-4-one

To a solution of (S)—N-[(1R)-1-[2-(5-fluoroisoindolin-2-yl)-3,6-dimethyl-4-oxo-chromen-8-yl]ethyl]-2-methyl-propane-2-sulfinamide (480 mg, 1.05 mmol, 1 eq) in EtOAc (3 mL) was added HCl/EtOAc (4 N, 5 mL) in one portion at 25° C. The mixture was stirred at 25° C. for 1 hr. The resulting suspension was directly filtered, the filter cake was dissolved in water (10 mL), adjusted pH=9 with concentrated NH₃·H₂O and filtered. The filter cake was dried in vacuo to give 8-[(1R)-1-aminoethyl]-2-(5-fluoroisoindolin-2-yl)-3,6-dimethyl-chromen-4-one (300 mg, 851.30 μmol, 80.98% yield) as a white solid. ¹H NMR (DMSO-d₆, 400 MHz) δ 7.61 (d, J=7.6 Hz, 2H), 7.45-7.44 (m, 1H), 7.30 (d, J=8.0 Hz, 1H), 7.17 (t, J=8.8 Hz, 1H), 5.16-5.10 (m, 4H), 4.59-4.54 (m, 1H), 2.38 (s, 3H), 2.22 (s, 3H), 1.35 (d, J=6.4 Hz, 3H).

### Preparation of (R)-8-(1-((2-bromo-4-chlorophenyl)amino)ethyl)-2-(5-fluoroisoindolin-2-yl)-3,6-dimethyl-4H-chromen-4-one

The title compound was prepared as a yellow solid (400 mg, 738.23 μmol, 86.72% yield) according to the similar procedure in Example 78, using 8-[(1R)-1-aminoethyl]-2-(5-fluoroisoindolin-2-yl)-3,6-dimethyl-chromen-4-one (300 mg, 851.30 μmol, 1 eq) and 2-bromo-4-chloro-1-iodo-benzene (405 mg, 1.28 mmol, 1.5 eq). ¹H NMR (DMSO-d₆, 400 MHz) δ 7.63 (d, J=1.2 Hz, 1H), 7.53 (d, J=2.4 Hz, 1H), 7.45-7.42 (m, 1H), 7.38-7.37 (m, 1H), 7.32-7.31 (m, 1H), 7.27-7.25 (m, 1H), 7.19-7.17 (m, 1H), 7.12-7.01 (m, 1H), 5.51 (d, J=6.8 Hz, 1H), 5.20-5.10 (m, 4H), 2.31 (s, 3H), 2.25 (s, 3H), 1.64 (d, J=9.2 Hz, 1H).

### Preparation of (R)-8-(1-((4-chloro-2-(4,4,5,5-tetramethyl-1,3,2-dioxaborolan-2-yl) phenyl)amino)ethyl)-2-(5-fluoroisoindolin-2-yl)-3,6-dimethyl-4H-chromen-4-one

The title compound was prepared as a yellow solid (400 mg, crude) according to the procedure described in preceding examples, using 8-[(1R)-1-(2-bromo-4-chloro-anilino)ethyl]-2-(5-fluoroisoindolin-2-yl)-3,6-dimethyl-chromen-4-one (400 mg, 738.23 μmol, 1 eq) and B₂Pin₂ (375 mg, 1.48 mmol, 2 eq). The product was used directly for next step without further purification.

### Preparation of (R)-8-(1-((4-chloro-2-(1-hydroxy-1H-benzo[d][1,2,6]oxazaborinin-6-yl)phenyl)amino)ethyl)-2-(5-fluoroisoindolin-2-yl)-3,6-dimethyl-4H-chromen-4-one

The title compound was prepared as a white solid (2.14 mg, 3.52 μmol, 1.38% yield) according to the procedures described herein and the synthetic scheme, using 8-[(1R)-1-[4-chloro-2-(4,4,5,5-tetramethyl-1,3,2-dioxaborolan-2-yl)anilino]ethyl]-2-(5-fluoroisoindolin-2-yl)-3,6-dimethyl-chromen-4-one (150 mg, 254.71 μmol, 1 eq) and 6-bromo-1-hydroxy-2,3,1-benzoxazaborinine (115 mg, 509.42 mol, 2 eq). ¹H NMR (DMSO-d₆, 400 MHz) δ 9.39 (s, 1H), 8.61 (s, 1H), 8.10 (d, J=8.0 Hz, 1H), 7.82-7.80 (m, 2H), 7.61 (s, 1H), 7.42-7.39 (m, 2H), 7.19-7.16 (m, 1H), 7.14-7.13 (m, 2H), 7.07-7.06 (m, 1H), 8.58 (d, J=8.8 Hz, 1H), 5.32-5.30 (m, 1H), 5.13-4.97 (m, 5H), 2.32 (s, 3H), 2.24 (s, 3H), 1.48 (d, J=4.4 Hz, 3H). MS (ESI): mass calcd. For C₃₄H₂₈BClFN₃O₄ 607.87, m/z found 608.2 [M+H]⁺. HPLC: 98.03% (220 nm), 99.73% (254 nm). Chiral purity: 97.14% ee.

### Example 25

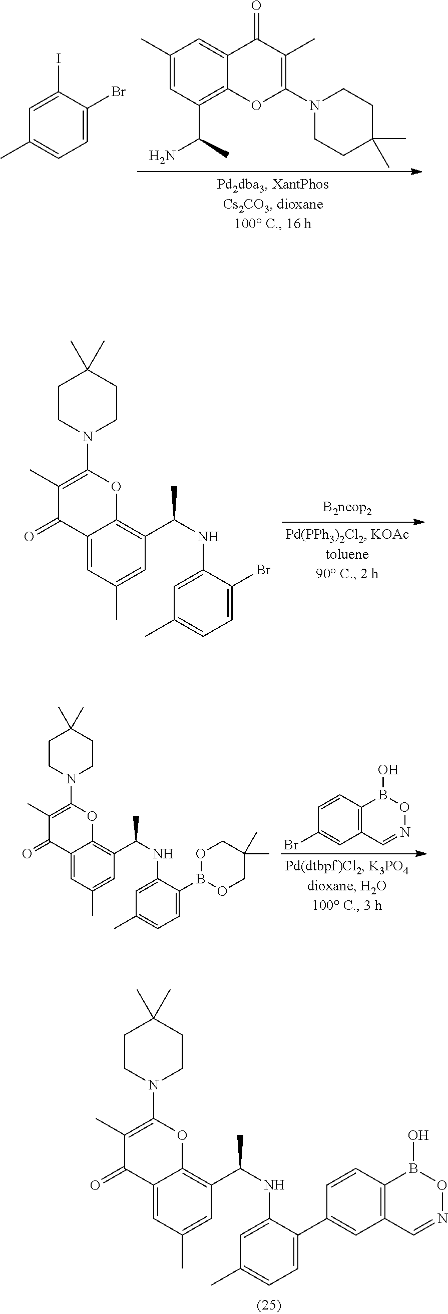

### Preparation of 8-[(1R)-1-(2-bromo-5-methyl-anilino)ethyl]-2-(4,4-dimethyl-1-piperidyl)-3,6-dimethyl-chromen-4-one

The title compound was prepared as a yellow solid (600 mg, 1.21 mmol, 79.23% yield) according to the similar procedure in Example 78, using 8-[(1R)-1-aminoethyl]-2-(4,4-dimethyl-1-piperidyl)-3,6-dimethyl-chromen-4-one (0.5 g, 1.52 mmol, 1 eq) and 1-bromo-2-iodo-4-methyl-benzene (904 mg, 3.04 mmol, 2 eq). ¹H NMR (CDCl₃, 400 MHz) δ 7.89 (d, J=1.2 Hz, 1H), 7.39 (d, J=2.0 Hz, 1H), 7.30 (d, J=8.0 Hz, 1H), 6.38 (dd, J=1.2, 8.0 Hz, 1H), 6.19 (d, J=1.2 Hz, 1H), 5.00-4.98 (m, 1H), 4.67-4.66 (s, 1H), 3.42-3.39 (m, 4H), 2.38 (s, 3H), 2.12 (s, 3H), 2.07 (s, 3H), 1.66 (d, J=6.8 Hz, 3H), 1.53-1.50 (m, 4H), 1.03 (s, 6H).

### Preparation of (R)-8-(1-((2-(5,5-dimethyl-1,3,2-dioxaborinan-2-yl)-5-methylphenyl)amino)ethyl)-2-(4,4-dimethylpiperidin-1-yl)-3,6-dimethyl-4H-chromen-4-one

The title compound was prepared as a yellow solid (500 mg, crude) according to the procedure described in preceding examples, using 8-[(1R)-1-(2-bromo-5-methyl-anilino)ethyl]-2-(4,4-dimethyl-1-piperidyl)-3,6-dimethyl-chromen-4-one (500 mg, 1.01 mmol, 1 eq) and B₂neop₂ (1.14 g, 5.03 mmol, 5 eq).

The product was used directly for next step without further purification.

### Preparation of 2-(4,4-dimethyl-1-piperidyl)-8-[(1R)-1-[2-(1-hydroxy-2,1-benzoxaborinin-6-yl)-5-methyl-anilino]ethyl]-3,6-dimethyl-chromen-4-one

The title compound was prepared as a white solid (31.4 mg, 55.72 μmol, 20.93% yield) according to the procedures described herein and the synthetic scheme, using 8-[(1R)-1-[2-(5,5-dimethyl-1,3,2-dioxaborinan-2-yl)-5-methyl-anilino]ethyl]-2-(4,4-dimethyl-1-piperidyl)-3,6-dimethyl-chromen-4-one (400 mg, 754 μmol, 1 eq) and 6-bromo-1-hydr oxy-2,3,1-benzoxazaborinine (170 mg, 754 μmol, 1 eq). ¹H NMR (DMSO-d₆, 400 MHz) δ 9.38 (s, 1H), 8.61 (s, 1H), 8.10 (d, J=7.6 Hz, 1H), 7.78-7.75 (m, 2H), 7.60 (s, 1H), 7.50 (d, J=2.0 Hz, 1H), 6.93 (d, J=7.2 Hz, 1H), 6.53 (d, J=7.6 Hz, 1H), 6.44 (s, 1H), 5.02-5.00 (m, 1H), 4.98-4.91 (m, 1H), 3.29-3.20 (m, 4H), 2.33 (s, 3H), 2.14 (s, 3H), 1.89 (s, 3H), 1.45 (d, J=6.8 Hz, 3H), 1.44-1.38 (m, 4H), 0.95 (s, 6H). MS (ESI): mass calcd. For C₃₄H₃₈BN₃O₄ 563.51, m/z found 564.3 [M+H]⁺. HPLC: 98.73% (220 nm), 99.05% (254 nm). Chiral purity: 100% ee.

### Example 26

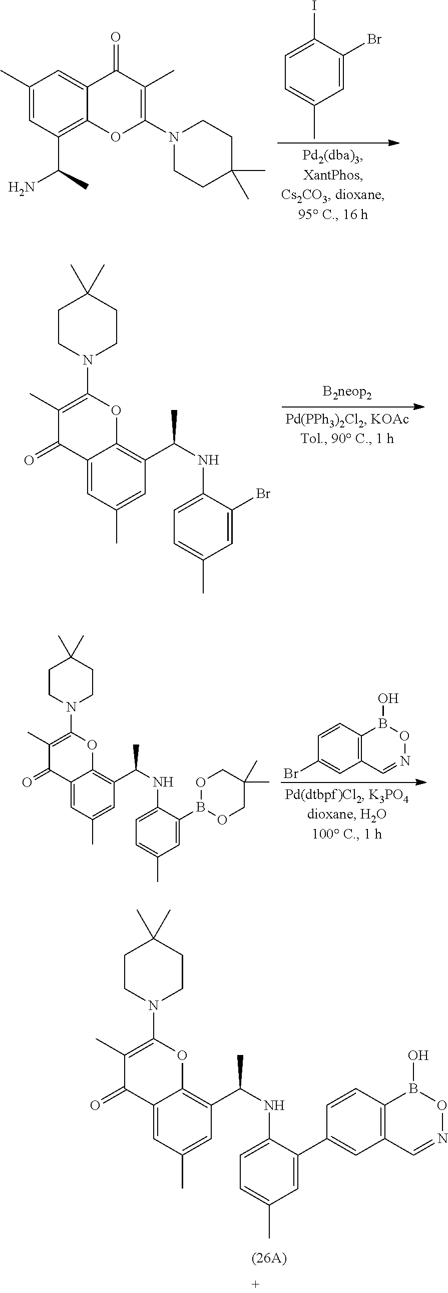

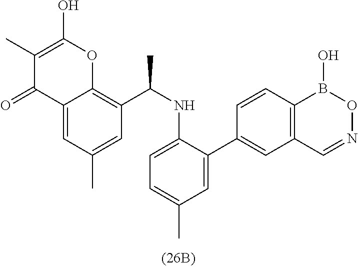 [SMILES: Cc1ccc(N[C@H](C)c2cc(C)cc3c(=O)c(C)c(O)oc23)c(-c2ccc3c(c2)C=NOB3O)c1]

### Preparation of 8-[(1R)-1-(2-bromo-4-methyl-anilino)ethyl]-2-(4,4-dimethyl-1-piperidyl)-3,6-dimethyl-chromen-4-one

The title compound was prepared as a yellow solid (200 mg, 402 μmol, 66.0% yield) according to the similar procedure in Example 78, using 2-bromo-1-iodo-4-methyl-benzene (361 mg, 1.22 mmol, 2 eq) and 8-[(1R)-1-aminoethyl]-2-(4,4-dimethyl-1-piperidyl)-3,6-dimethyl-chromen-4-one (200 mg, 608 mol, 1 eq). ¹H NMR (CDCl₃, 400 MHz) δ 7.87 (d, J=1.2 Hz, 1H), 7.37 (d, J=2.0 Hz, 1H), 7.29-7.27 (m, 1H), 6.82 (dd, J=1.2, 8.4 Hz, 1H), 6.23 (d, J=8.4 Hz, 1H), 4.96 (d, J=6.4 Hz, 1H), 4.59 (s, 1H), 3.43-3.35 (m, 4H), 2.36 (s, 3H), 2.18 (s, 3H), 2.06 (s, 3H), 1.67-1.63 (m, 3H), 1.55-1.50 (m, 4H), 1.04 (s, 6H)

### Preparation of 8-[(1R)-1-[2-(5,5-dimethyl-1,3,2-dioxaborinan-2-yl)-4-methyl-anilino]ethyl]-2-(4,4-dimethyl-1-piperidyl)-3,6-dimethyl-chromen-4-one

The title compound was prepared as yellow oil (200 mg, 377 μmol, 93.7% yield) according to the procedures described herein and the synthetic scheme, using 8-[(1R)-1-(2-bromo-4-methyl-anilino)ethyl]-2-(4,4-dimethyl-1-piperidyl)-3,6-dimethyl-chromen-4-one (200 mg, 402 μmol, 1 eq) and B₂neop₂ (454 mg, 2.01 mmol, 5 eq). ¹H NMR (CDCl₃, 400 MHz) δ 7.84 (s, 1H), 7.50 (d, J=2.0 Hz, 1H), 7.45 (s, 1H), 6.93 (d, J=7.2 Hz, 1H), 6.38 (s, 1H), 6.16 (s, 1H), 5.05-4.83 (m, 1H), 3.83 (s, 2H), 3.66 (s, 2H), 3.43-3.34 (m, 4H), 2.35 (s, 3H), 2.18 (s, 3H), 2.06 (d, J=1.2 Hz, 3H), 1.59 (s, 3H), 1.54-1.48 (m, 4H), 1.07 (s, 6H), 1.03 (s, 6H)

### Preparation of 2-(4,4-dimethyl-1-piperidyl)-8-[(R)-1-[2-(1-hydroxy-2,3,1-benzoxazaborinin-6-yl)-4-methyl-anilino]ethyl]-3,6-dimethyl-chromen-4-one and by-product 2-hydroxy-8-[(1R)-1-[2-(1-hydroxy-2,3,1-benzoxazaborinin-6-yl)-4-methyl-anilino]ethyl]-3,6-dimethyl-chromen-4-one

The title compound was prepared as a white solid (18 mg, 31.9 μmol, 12.7% yield) according to the procedures described herein and the synthetic scheme, using 8-[(1R)-1-[2-(5,5-dimethyl-1,3,2-dioxaborinan-2-yl)-4-methyl-anilino]ethyl]-2-(4,4-dimethyl-1-piperidyl)-3,6-dimethyl-chromen-4-one (200 mg, 377 μmol, 1.5 eq) and 6-bromo-1-hydroxy-2,3,1-benzoxazaborinine (56.7 mg, 251 μmol, 1 eq) in H₂O (0.4 mL). ¹H NMR (DMSO-d₆, 400 MHz) δ 9.43 (s, 1H), 8.64 (s, 1H), 8.12 (d, J=7.6 Hz, 1H), 7.83-7.80 (m, 2H), 7.59 (s, 1H), 7.45 (d, J=1.6 Hz, 1H), 6.91-6.82 (m, 2H), 6.49 (d, J=8.4 Hz, 1H), 4.94-4.85 (m, 2H), 3.27-3.20 (m, 4H), 2.32 (s, 3H), 2.10 (s, 3H), 1.89 (s, 3H), 1.49-1.41 (m, 3H), 1.41-1.33 (m, 4H), 0.95 (s, 6H). MS (ESI): mass calcd. For C₃₄H₃₈BN₃O₄ 563.30, m/z found 564.3 [M+H]⁺. HPLC: 97.62% (220 nm), 98.10% (254 nm). Chiral purity: 97.14% ee. A by-product 2-hydroxy-8-[(1R)-1-[2-(1-hydroxy-2,3,1-benzoxazaborinin-6-yl)-4-methyl-anilino]ethyl]-3,6-dimethyl-chromen-4-one was also isolated (6.5 mg, 13.8 μmol, 5.5% yield) as a yellow solid. ¹H NMR (DMSO-d₆, 400 MHz) δ 9.41 (s, 1H), 8.69 (s, 1H), 8.15 (d, J=7.6 Hz, 1H), 7.83 (dd, J=1.6, 7.6 Hz, 1H), 7.80 (s, 1H), 7.54 (d, J=1.6 Hz, 1H), 7.35 (d, J=2.0 Hz, 1H), 6.90-6.85 (m, 2H), 6.40 (d, J=8.4 Hz, 1H), 4.93-4.78 (m, 2H), 2.30 (s, 3H), 2.14 (s, 3H), 1.98 (s, 3H), 1.37 (d, J=6.4 Hz, 3H). MS (ESI): mass calcd. For C₂₇H₂₅BN₂O₅ 468.19, m/z found 469.3 [M+H]⁺. HPLC: 93.30% (220 nm), 93.19% (254 nm).

### Example 27

### Preparation of 8-acetyl-3,6-dimethyl-2-morpholino-chromen-4-one

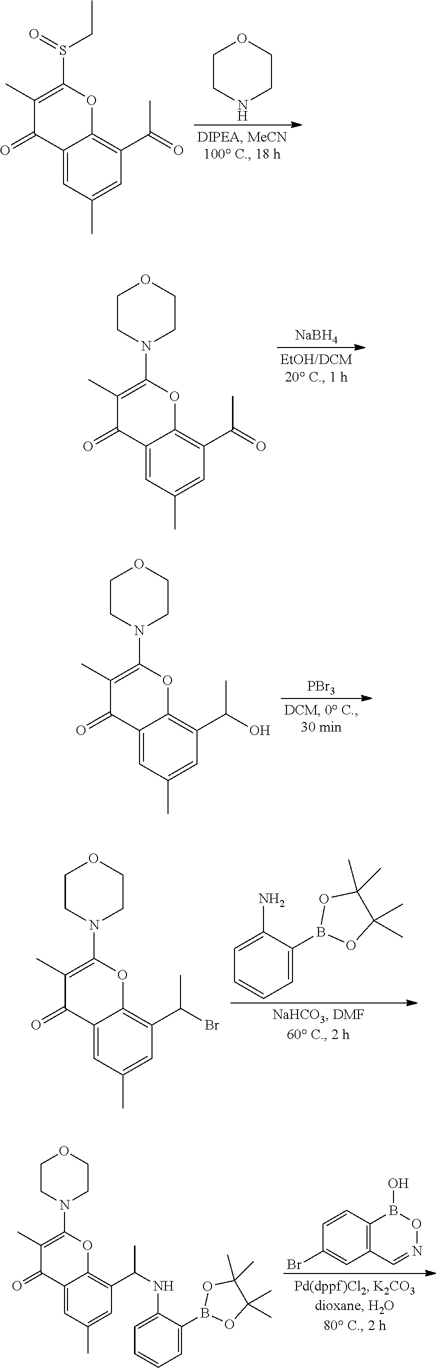

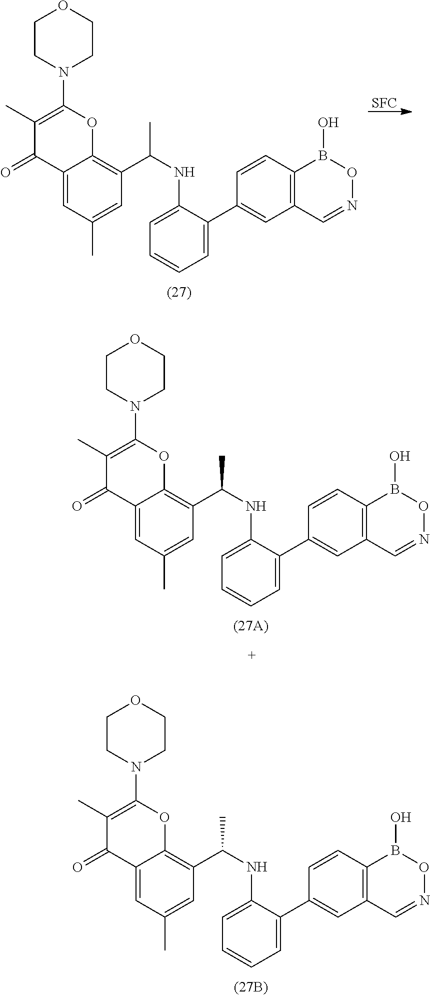

To a solution of 8-acetyl-2-ethylsulfinyl-3,6-dimethyl-chromen-4-one (1.00 g, 3.42 mmol, 1 eq) and morpholine (745 mg, 8.55 mmol, 2.5 eq) in MeCN (20 mL) was added DIEA (1.33 g, 10.3 mmol, 1.79 mL, 3 eq) in one portion at 20° C., the reaction mixture was heated to 100° C. and stirred at 100° C. for 18 h. The reaction was cooled to 25° C. and then some solid formed. The solid was filtered, triturated with MTBE (10 mL) and dried in vacuo to give the title compound (700 mg, 2.32 mmol, 67.91% yield) as a yellow solid. ¹H NMR (DMSO-d₆, 400 MHz) δ 7.98 (d, J=1.6 Hz, 1H), 7.90 (d, J=2.4 Hz, 1H), 3.75-3.72 (m, 4H), 3.46-3.42 (m, 4H), 2.70 (s, 3H), 2.43 (s, 3H), 1.93 (s, 3H).

### Preparation of 8-(1-hydroxyethyl)-3,6-dimethyl-2-morpholino-chromen-4-one

To a solution of 8-acetyl-3,6-dimethyl-2-morpholino-chromen-4-one (1.15 g, 3.82 mmol, 1 eq) in EtOH (10 mL)/DCM (10 mL) was added NaBH₄ (320 mg, 8.46 mmol, 2.2 eq) in portions at 20° C. under N₂, the reaction mixture was warmed to 20° C. and stirred at 20° C. for 1 h. The mixture was poured into ice water (35 mL) at 0° C., nitrogen atmosphere protection was applied. The mixture was warmed up to 20° C. and then extracted with DCM (20 mL×3). The combined organic phase was washed with brine (25 mL×2), dried over Na₂SO₄, filtered and concentrated under reduced pressure to give the title compound (1.1 g, 3.63 mmol, 95.02% yield) as a yellow solid. ¹H NMR (DMSO-d₆, 400 MHz) δ 7.63 (s, 1H), 7.59 (d, J=2.0 Hz, 1H), 5.34 (d, J=4.4 Hz, 1H), 5.24-5.18 (m, 1H), 3.74 (t, J=4.4 Hz, 4H), 3.41-3.37 (m, 4H), 2.39 (s, 3H), 1.91 (s, 3H), 1.40 (d, J=6.4 Hz, 3H).

### Preparation of 8-(1-bromoethyl)-3,6-dimethyl-2-morpholino-chromen-4-one

To a solution of 8-(1-hydroxyethyl)-3,6-dimethyl-2-morpholino-chromen-4-one (1.50 g, 4.94 mmol, 1 eq) in DCM (20 mL) was added dropwise PBr₃ (1.61 g, 5.93 mmol, 1.2 eq) at 0° C. under N₂, the reaction mixture was stirred 0° C. for 30 min. The reaction mixture was poured into ice (20 g), adjusted pH=7 with sat.aq.NaHCO₃ at 0° C. and extracted with DCM (30 mL×3). The combined organic layers were washed with brine (30 mL×2), dried over Na₂SO₄, filtered and concentrated under reduced pressure to give a residue. The residue was purified by flash silica gel chromatography (BIOTAGE®; 20 g SepaFlash® Silica Flash Column, Eluent of 0~100% Ethyl acetate/Petroleum ether gradient@60 m/min) to give the title compound (450 mg, 1.23 mmol, 24.85% yield) as a pale yellow solid. ¹H NMR (CDCl₃, 400 MHz) δ 7.97 (s, 1H), 7.53 (s, 1H), 5.64 (q, J=3.2 Hz, 1H), 3.89 (t, J=4.4 Hz, 4H), 3.51 (t, J=4.4 Hz, 4H), 2.46 (s, 3H), 2.17 (d, J=6.8 Hz, 1H), 2.07 (s, 3H).

### Preparation of 3,6-dimethyl-2-morpholino-8-[1-[2-(4,4,5,5-tetramethyl-1,3,2-dioxaborolan-2-yl)anilino]ethyl]chromen-4-one

To a solution of 8-(1-bromoethyl)-3,6-dimethyl-2-morpholino-chromen-4-one (450 mg, 1.23 mmol, 1 eq) and 2-(4,4,5,5-tetramethyl-1,3,2-dioxaborolan-2-yl)aniline (450 mg, 2.05 mmol, 1.67 eq) in DMF (10 mL) was added NaHCO₃ (155 mg, 1.84 mmol, 1.5 eq) and 25° C., the reaction mixture was heated to 60° C. and stirred at 60° C. for 2 h. The reaction mixture was cooled to 0° C., poured into ice-water (40 mL), diluted with DCM 15 mL and extracted with DCM (15 mL×3). The combined organic layers were washed with brine (25 mL×3), dried over Na₂SO₄, filtered and concentrated under reduced pressure to give a residue. The residue was purified by flash silica gel chromatography (BIOTAGE®; 20 g SepaFlash® Silica Flash Column, Eluent of 0~100% Ethyl acetate/Petroleum ether gradient@75 mL/min) to give the title compound (500 mg, 991.23 μmol, 80.67% yield) as a pale yellow solid. ¹H NMR (CDCl₃, 400 MHz) δ 7.87 (s, 1H), 7.66 (dd, J=1.6, 7.2 Hz, 1H), 7.49 (d, J=2.0 Hz, 1H), 7.17-7.12 (m, 1H), 6.64 (t, J=7.2 Hz, 1H), 6.36 (br s, 1H), 6.25 (d, J=8.0 Hz, 1H), 5.02-4.95 (m, 1H), 3.87-3.82 (m, 4H), 3.43-3.40 (m, 4H), 2.38 (s, 3H), 2.07 (s, 3H), 1.60 (d, J=6.8 Hz, 3H), 1.38 (s, 12H).

### Preparation of 8-[1-[2-(1-hydroxy-2,3,1-benzoxazaborinin-6-yl)anilino]ethyl]-3,6-di methyl-2-morpholino-chromen-4-one

To a solution of 3,6-dimethyl-2-morpholino-8-[1-[2-(4,4,5,5-tetramethyl-1,3,2-dioxaborolan-2-yl)anilino]ethyl]chromen-4-one (180 mg, 357 μmol, 1 eq) and 6-bromo-1-hydroxy-2,3,1-benzoxazaborinine (105 mg, 464 μmol, 1.3 eq) in dioxane (3.2 mL)/H₂O (0.4 mL) were added Pd(dppf)Cl₂ (26 mg, 35.7 μmol, 0.1 eq) and K₂CO₃ (99 mg, 713 μmol, 2 eq) in turns at 25° C. The reaction mixture was degassed and purged with N₂ for 3 times, and then stirred at 80° C. (the oil bath was pre-heated to 80° C.) for 2 h under N₂ atmosphere. The reaction mixture was cooled to 25° C. and filtered.

The filtrate was directly purified by prep-HPLC (column: Waters Xbridge BEH C18 100×30 mm×10 um; mobile phase: [H2O (10 mM NH4HCO3)-ACN]; gradient:22%-50% B over 8.0 min) to give the title compound (38.1 mg, 20.20% yield) as a white solid. ¹H NMR (DMSO-d₆, 400 MHz) δ 9.39 (s, 1H), 8.66 (s, 1H), 8.15 (d, J=7.6 Hz, 1H), 7.87-7.83 (m, 1H), 7.82 (s, 1H), 7.62 (d, J=1.2 Hz, 1H), 7.49 (d, J=1.2 Hz, 1H), 7.10 (t, J=7.2 Hz, 1H), 7.07-7.03 (m, 1H), 6.71 (t, J=7.6 Hz, 1H), 6.57 (d, J=8.0 Hz, 1H), 5.05 (d, J=7.2 Hz, 1H), 4.97-4.90 (m, 1H), 3.66 (t, J=4.8 Hz, 4H), 3.28-3.19 (m, 4H), 2.33 (s, 3H), 1.91 (s, 3H), 1.45 (d, J=6.8 Hz, 3H). MS (ESI): mass calcd. For C₃₀H₃₀BN₃O₅ 523.23, m/z found 524.2 [M+H]⁺. HPLC: 99.02% (220 nm), 99.55% (254 nm).

### Preparation of 8-[(1S)-1-[2-(1-hydroxy-2,3,1-benzoxazaborinin-6-yl)anilino]ethyl]-3,6-dimethyl-2-morpholino-chromen-4-one and 8-[(1R)-1-[2-(1-hydroxy-2,3,1-benzoxazaborinin-6-yl)anilino]ethyl]-3,6-dimethyl-2-morpholino-chromen-4-one

8-[1-[2-(1-hydroxy-2,3,1-benzoxazaborinin-6-yl)anilino]ethyl]-3,6-dimethyl-2-morpholino-chromen-4-one (111 mg) was separated by SFC (column: DAICEL CHIRALPAK IG (250 mm×30 mm, 10 um); mobile phase: [CO2-IPA]; B %:50%, isocratic elution mode) to give the title compound, Isomer 1 (41.6 mg, 36.74% yield) as a white solid and the title compound, Isomer 2 (40.5 mg, 36.24% yield) as a white solid. Isomer 1: ¹H NMR (DMSO-d₆, 400 MHz) δ 9.41 (s, 1H), 8.66 (s, 1H), 8.15 (d, J=7.6 Hz, 1H), 7.85 (d, J=7.6 Hz, 1H), 7.82 (s, 1H), 7.61 (d, J=1.2 Hz, 1H), 7.49 (d, J=1.2 Hz, 1H), 7.10-7.03 (m, 2H), 6.71 (t, J=7.2 Hz, 1H), 6.57 (d, J=8.0 Hz, 1H), 5.05 (d, J=7.2 Hz, 1H), 4.97-4.90 (m, 1H), 3.70-3.65 (m, 4H), 3.28-3.18 (m, 4H), 2.33 (s, 3H), 1.90 (s, 3H), 1.45 (d, J=6.4 Hz, 3H). MS (ESI): mass calcd. For C₃₀H₃₀BN₃O₅ 523.23, m/z found 524.3 [M+H]⁺. HPLC: 98.04% (220 nm), 98.79% (254 nm). Chiral purity: 100% ee. Isomer 2: ¹H NMR (DMSO-d₆, 400 MHz) δ 9.40 (s, 1H), 8.66 (s, 1H), 8.15 (d, J=7.6 Hz, 1H), 7.87-7.83 (m, 1H), 7.82 (s, 1H), 7.62 (s, 1H), 7.49 (d, J=1.2 Hz, 1H), 7.15-7.03 (m, 2H), 6.71 (t, J=7.2 Hz, 1H), 6.57 (d, J=8.0 Hz, 1H), 5.05 (d, J=7.6 Hz, 1H), 4.97-4.90 (m, 1H), 3.66 (t, J=4.8 Hz, 4H), 3.28-3.19 (m, 4H), 2.33 (s, 3H), 1.91 (s, 3H), 1.45 (d, J=6.8 Hz, 3H). MS (ESI): mass calcd. For C₃₀H₃₀BN₃O₅ 523.23, m/z found 524.3 [M+H]⁺. HPLC: 99.33% (220 nm), 99.62% (254 nm). Chiral purity: 98.46% ee.

### Example 28

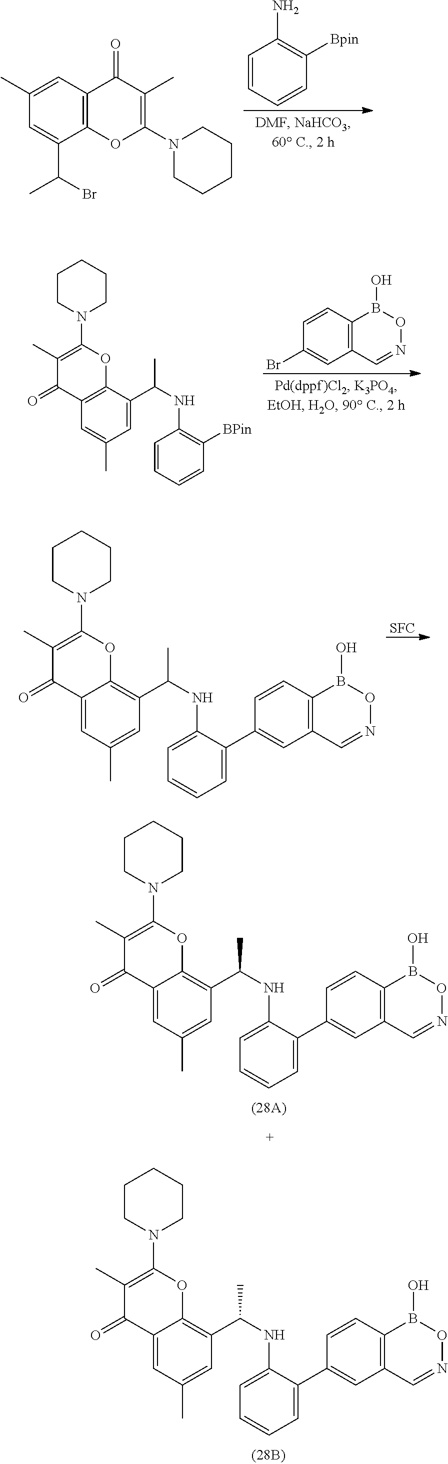

### Preparation of 3,6-dimethyl-2-(piperidin-1-yl)-8-(1-((2-(4,4,5,5-tetramethyl-1,3,2-dioxaborolan-2-yl)phenyl)amino)ethyl)-4H-chromen-4-one

A mixture of 8-(1-bromoethyl)-3,6-dimethyl-2-(1-piperidyl)chromen-4-one (500 mg, 1.37 mmol, 1 eq), 2-(4,4,5,5-tetramethyl-1,3,2-dioxaborolan-2-yl)aniline (601 mg, 2.75 mmol, 2 eq) and NaHCO₃ (172 mg, 2.06 mmol, 80 μL, 1.5 eq) in DMF (10 mL) was degassed and purged with N₂ for 3 times at 20° C., and then the mixture was heated to 60° C. stirred at 60° C. for 2 h under N₂ atmosphere. The reaction mixture was cooled to 20° C., poured into H₂O (50 mL) and then filtered. The filter cake was collected and dried in vacuo to give the title compound (1.0 g, crude) as a yellow solid. ¹H NMR (CDCl₃, 400 MHz) δ7.84 (s, 1H), 7.65-7.63 (m, 1H), 7.44 (s, 1H), 7.13 (t, J=7.2 Hz, 1H), 6.69-6.59 (m, 1H), 6.34-6.33 (m, 1H), 6.23-6.21 (m, 1H), 5.03-4.98 (m, 1H), 3.39-3.37 (m, 4H), 2.35 (s, 3H), 2.05 (s, 3H), 1.07 (s, 6H), 1.58 (d, J=6.4 Hz, 3H), 1.37 (s, 12H).

### Preparation of 8-(1-((2-(1-hydroxy-1H-benzo[d][1,2,6]oxazaborinin-6-yl)phenyl)amino) ethyl)-3,6-dimethyl-2-(piperidin-1-yl)-4H-chromen-4-one

A mixture of 3,6-dimethyl-2-(piperidin-1-yl)-8-(1-((2-(4,4,5,5-tetramethyl-1,3,2-dioxaborolan-2-yl)phenyl) amino)ethyl)-4H-chromen-4-one (500 mg, 995 μmol, 1 eq), 6-bromo-1-hydroxy-2,3,1-benzoxazaborinine (224 mg, 995 μmol, 1 eq), K₃PO₄ (422 mg, 1.99 mmol, 2 eq) and Pd(dppf)Cl₂ (72 mg, 99.5 μmol, 0.1 eq) in EtOH (5 mL)/H₂O (1 mL) was degassed and purged with N₂ for 3 times at 20° C., and then the mixture was heated to 90° C. and stirred at 90° C. for 2 h under N₂ atmosphere. The reaction mixture was filtered and the filtrate was purified by prep-HPLC (column: Phenomenex Gemini-NX 80×40 mm×3 um; mobile phase: [H2O (10 mM □H4HCO3)-AC□]; gradient:20%-50% B over 8.0 min) to give the title compound (100 mg, 183.30 μmol, 18.42% yield) as a white solid.

### Preparation of (R)-8-(1-((2-(1-hydroxy-1H-benzo[d][1,2,6]oxazaborinin-6-yl)phenyl) amino)ethyl)-3,6-dimethyl-2-(piperidin-1-yl)-4H-chromen-4-one and (S)-8-(1-((2-(1-hydroxy-1H-benzo[d][1,2,6]oxazaborinin-6-yl)phenyl)amino)ethyl)-3,6-dimethyl-2-(piperidin-1-yl)-4H-chromen-4-one

8-(1-((2-(1-hydroxy-1H-benzo[d][1,2,6]oxazaborinin-6-yl)phenyl)amino)ethyl)-3,6-dimethyl-2-(piperidin-1-yl)-4H-chromen-4-one (100 mg, 183 μmol, 1 eq) was purified by SFC (column: DAICEL CHIRALCEL OD (250 mm×30 mm, 10 um); mobile phase: [CO2-IPA (0.1% □H₃H₂O)]; B %:50%, isocratic elution mode) to give the title compound, Isomer 1 (40.8 mg, 78.25 μmol, 42.33% yield) as a white solid and the title compound, Isomer 2 (40.2 mg, 77.10 μmol, 42.83% yield) as a white solid. Isomer 1: ¹H NMR (DMSO-d₆, 400 MHz) δ 9.45-9.40 (m, 1H), 8.65-8.61 (m, 1H), 8.13-8.13 (m, 1H), 7.87-7.81 (m, 1H), 7.60-7.58 (m, 1H), 7.47-7.43 (m, 2H), 7.08-7.03 (m, 2H), 6.71-6.68 (m, 1H), 6.55-6.45 (m, 1H), 5.04-4.82 (m, 2H), 3.22-3.22 (m, 4H), 2.31 (s, 3H), 1.89 (s, 3H), 1.56 (s, 6H), 1.45-1.44 (m, 3H). MS (ESI): mass calcd. For C₃₁H₃₂BN₃O₄ 521.42, m/z found 522.2 [M+H]⁺. HPLC: 98.66% (220 nm), 99.76% (254 nm). Chiral purity: 99.76% ee. Isomer 2: ¹H NMR (DMSO-d₆, 400 MHz) δ 8.19-8.05 (m, 1H), 7.94-7.84 (m, 1H), 7.60-7.58 (m, 2H), 7.43-7.39 (m, 2H), 7.05-6.98 (m, 2H), 6.69-6.65 (m, 1H), 6.48-6.48 (m, 1H), 5.02-4.70 (m, 2H), 3.25-3.25 (m, 4H), 2.32-2.31 (m, 3H), 1.89 (s, 3H), 1.57 (s, 6H), 1.45-1.43 (m, 3H). MS (ESI): mass calcd. For C₃₁H₃₂BN₃O₄ 521.42, m/z found 522.2 [M+H]⁺. HPLC: 99.36% (220 nm), 99.53% (254 nm). Chiral purity: 99.88% ee.

### Example 29

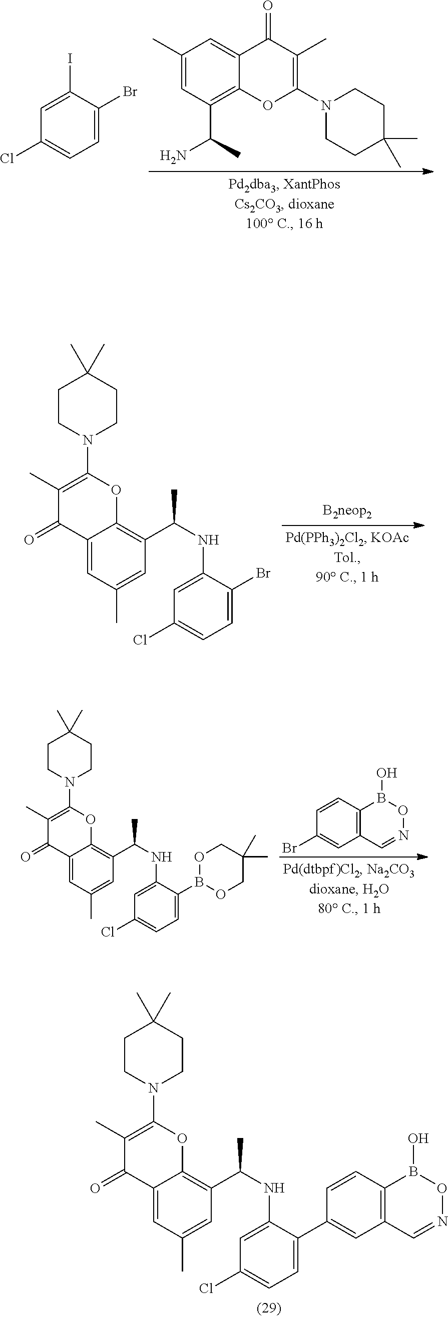

### Preparation of 8-[(1R)-1-(2-bromo-5-chloro-anilino)ethyl]-2-(4,4-dimethyl-1-piperidyl)-3,6-dimethyl-chromen-4-one

The title compound was prepared as a yellow solid (500 mg, 79.3% yield) according to the similar procedure in Example 78, using 8-[(1R)-1-aminoethyl]-2-(4,4-dimethyl-1-piperidyl)-3,6-dimethyl-chromen-4-one (400 mg, 1.22 mmol, 1 eq) and 1-bromo-4-chloro-2-iodo-benzene (772 mg, 2.44 mmol, 2 eq). ¹H NMR (CDCl₃, 400 MHz) δ 7.91 (d, J=1.2 Hz, 1H), 7.38-7.30 (m, 2H), 6.53 (dd, J=2.4, 8.4 Hz, 1H), 6.39 (d, J=2.4 Hz, 1H), 4.98-4.95 (m, 1H), 4.78 (d, J=6.0 Hz, 1H), 3.45-3.33 (m, 4H), 2.40 (s, 3H), 2.11-2.02 (m, 3H), 1.66 (d, J=6.8 Hz, 3H), 1.57-1.48 (m, 4H), 1.04 (s, 6H)

### Preparation of 8-[(1R)-1-[5-chloro-2-(5,5-dimethyl-1,3,2-dioxaborinan-2-yl)anilino]ethyl]-2-(4,4-dimethyl-1-piperidyl)-3,6-dimethyl-chromen-4-one

The title compound was prepared as a yellow solid (500 mg, 907 μmol, 94.0% yield) according to the procedures described herein and the synthetic scheme, using 8-[(1R)-1-(2-bromo-5-chloro-anilino)ethyl]-2-(4,4-dimethyl-1-piperidyl)-3,6-dimethyl-chromen-4-one (500 mg, 965 μmol, 1 eq) and B₂neop₂ (1.09 g, 4.83 mmol, 5 eq). ¹H NMR (CDCl₃, 400 MHz) δ 7.89 (d, J=1.6 Hz, 1H), 7.59 (d, J=8.0 Hz, 1H), 7.38 (d, J=2.0 Hz, 1H), 6.76-6.51 (m, 2H), 6.30 (d, J=1.6 Hz, 1H), 5.00-4.90 (m, 1H), 3.66 (s, 4H), 3.46-3.37 (m, 4H), 2.38 (s, 3H), 2.11-2.07 (m, 3H), 1.58 (d, J=6.8 Hz, 3H), 1.55-1.50 (m, 4H), 1.05 (d, J=5.6 Hz, 12H)

### Preparation of 8-[(1R)-1-[5-chloro-2-(1-hydroxy-2,3,1-benzoxazaborinin-6-yl)anilino]ethyl]-2-(4,4-dimethyl-1-piperidyl)-3,6-dimethyl-chromen-4-one

The title compound was prepared as a white solid (37 mg, 63.3 μmol, 17.4% yield) according to the procedures described herein and the synthetic scheme, using 8-[(1R)-1-[5-chloro-2-(5,5-dimethyl-1,3,2-dioxaborinan-2-yl)anilino]ethyl]-2-(4,4-dimethyl-1-piperidyl)-3,6-dimethyl-chromen-4-one (200 mg, 363 μmol, 1 eq) and 6-bromo-1-hydroxy-2,3,1-benzoxazaborinine (122 mg, 544 μmol, 1.5 eq). ¹H NMR (DMSO-d₆, 400 MHz) δ 9.42 (s, 1H), 8.64 (s, 1H), 8.13 (d, J=8.0 Hz, 1H), 7.81-7.77 (m, 2H), 7.63 (s, 1H), 7.51 (d, J=2.0 Hz, 1H), 7.05 (d, J=8.0 Hz, 1H), 6.73 (dd, J=1.6, 8.0 Hz, 1H), 6.59 (d, J=1.6 Hz, 1H), 5.43 (d, J=7.2 Hz, 1H), 4.99-4.93 (m, 1H), 3.29-3.21 (m, 4H), 2.33 (s, 3H), 1.91 (s, 3H), 1.47 (d, J=6.8 Hz, 3H), 1.42 (s, 4H), 0.97 (s, 6H). MS (ESI): mass calcd. For C₃₃H₃₅BClN₃O₄ 583.24, m/z found 584.3 [M+H]⁺. HPLC: 97.41% (220 nm), 97.57% (254 nm). Chiral purity: 98.1% ee.

### Example 30

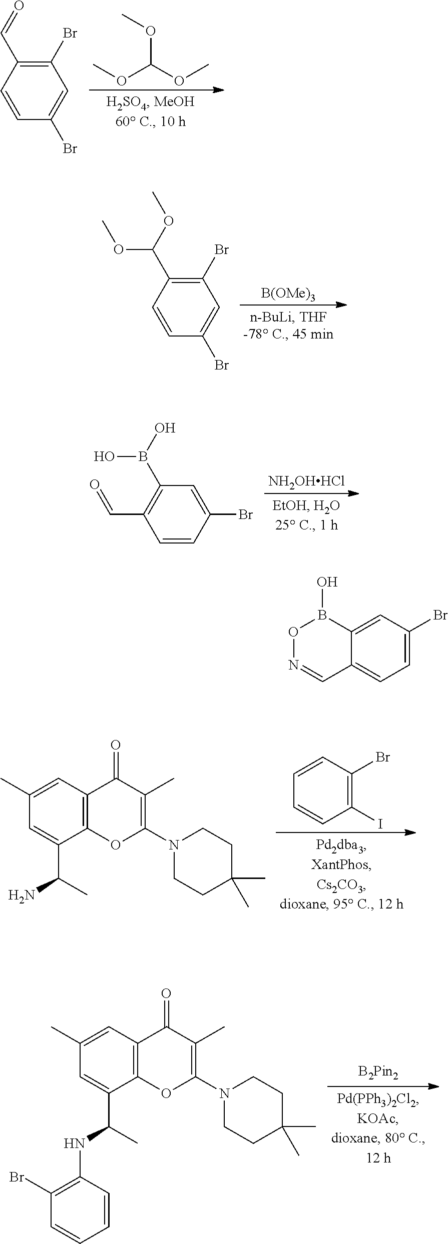

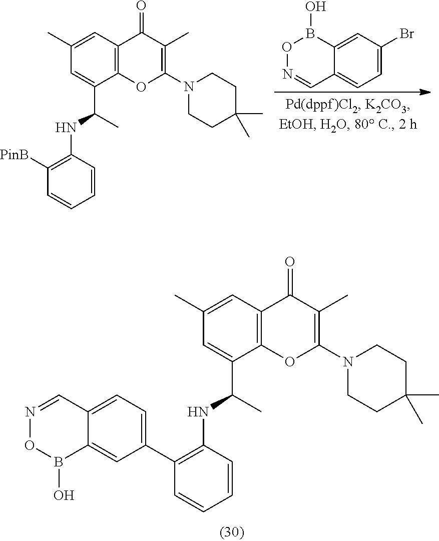

### Preparation of 2,4-dibromo-1-(dimethoxymethyl)benzene

To a solution of 2,4-dibromobenzaldehyde (2.00 g, 7.58 mmol, 1 eq) and trimethoxymethane (1.05 g, 9.85 mmol, 1.08 mL, 1.3 eq) in MeOH (20 mL) was added H₂SO₄ (75.84 mg, 757.82 μmol, 41.22 μL, 0.1 eq) dropwise at 25° C., the reaction mixture was heated to 60° C. and stirred at 60° C. for 10 h. The reaction mixture was cooled to 0° C., quenched with sat.aq NaHCO₃ (15 mL) and extracted with EtOAc (20 mL×3). The combined organic layers were washed with brine (15 mL×2), dried over Na₂SO₄, filtered and concentrated under reduced pressure to give the title compound (2.30 g, 7.42 mmol, 97.91% yield) as colorless oil. ¹H NMR (CDCl₃, 400 MHz) δ 7.74 (s, 1H), 7.49-7.47 (m, 2H), 5.51 (s, 1H), 3.38 (s, 6H).

### Preparation of (5-bromo-2-formyl-phenyl)boronic acid

To a solution of 2,4-dibromo-1-(dimethoxymethyl)benzene (0.50 g, 1.61 mmol, 1 eq) in THF (6 mL) was added n-BuLi (2.5 M, 645.20 μL, 1 eq) dropwise at −78° C., the reaction mixture was stirred at −78° C. for 15 min. Then trimethyl borate (168 mg, 1.61 mmol, 182.19 μL, 1 eq) was added dropwise and then the reaction mixture was stirred at −78° C. for additional 30 min. The reaction mixture was warmed to 0° C., poured into HCl (10 mL, 3 M) and extracted with EtOAc (10 mL×3). The combined organic phase was washed with brine (10 mL), dried over Na₂SO₄, filtered and concentrated in vacuum. The crude product was further triturated with EtOAc (8 mL) at 0° C. for 10 min to give the title compound (0.29 g, 1.27 mmol, 78.57% yield) as a light yellow solid. ¹H NMR (DMSO-d₆, 400 MHz) δ 10.09 (s, 1H), 8.38 (s, 2H), 7.80 (s, 1H), 7.78-7.77 (m, 1H), 7.75-7.73 (m, 1H).

### Preparation of 7-bromo-1-hydroxy-2,3,1-benzoxazaborinine

To a solution of (5-bromo-2-formyl-phenyl)boronic acid (0.27 g, 1.18 mmol, 1 eq) in EtOH (3 mL)/H₂O (0.3 mL) was added NH₂OH—HCl (164 mg, 2.36 mmol, 2 eq) in portions at 25° C., the reaction mixture was stirred at 25° C. for 1 h. The reaction suspension was directly filtered and the filter cake was dried in vacuo to give the title compound (0.24 g, 1.06 mmol, 90.07% yield) as a white solid, which was used directly for next step without further purification. ¹H NMR (DMSO-d₆, 400 MHz) δ 9.56 (br s, 1H), 8.67 (s, 1H), 8.23 (s, 1H), 8.02 (d, J=8.4 Hz, 1H), 7.72 (d, J=8.4 Hz, 1H).

### Preparation of 8-[(1R)-1-(2-bromoanilino)ethyl]-2-(4,4-dimethyl-1-piperidyl)-3,6-dimethyl-chromen-4-one

The title compound was prepared as a light yellow oil (11.00 g, 22.75 mmol, 83.04% yield) according to the similar procedure in Example 78, using 8-[(1R)-1-aminoethyl]-2-(4,4-dimethyl-1-piperidyl)-3,6-dimethyl-chromen-4-one (9.00 g, 27.40 mmol, 1 eq) and 1-bromo-2-iodo-benzene (15.50 g, 54.80 mmol, 7.04 mL, 2 eq). ¹H NMR (CDCl₃, 400 MHz) δ 7.63 (s, 1H), 7.47 (s, 1H), 7.43-7.41 (m, 1H), 7.04-7.02 (m, 1H), 6.53-6.49 (m, 1H), 6.45 (d, J=8.4 Hz, 1H), 5.29-5.27 (m, 1H), 5.05-5.02 (m, 1H), 3.42-3.39 (m, 4H), 2.31 (s, 3H), 1.91 (s, 3H), 1.61 (d, J=6.4 Hz, 3H), 1.47-1.44 (m, 4H), 0.98 (s, 6H).

### Preparation of 2-(4,4-dimethyl-1-piperidyl)-3,6-dimethyl-8-[(1R)-1-[2-(4,4,5,5-tetramethyl-1,3,2-dioxaborolan-2-yl)anilino]ethyl]chromen-4-one

The title compound was prepared as a light yellow solid (38.00 g, crude) according to the procedures described herein and the synthetic scheme, using 8-[(1R)-1-(2-bromoanilino)ethyl]-2-(4,4-dimethyl-1-piperidyl)-3,6-dimethyl-chromen-4-one (33.00 g, 68.26 mmol, 1 eq), B₂Pin₂ (52.00 g, 204.78 mmol, 3 eq). The product was used directly for next step without further purification.

### Preparation of 2-(4,4-dimethyl-1-piperidyl)-8-[(1R)-1-[2-(1-hydroxy-2,3,1-benzoxaza borinin-6-yl)anilino]ethyl]-3,6-dimethyl-chromen-4-one

The title compound was prepared as a white solid (56.5 mg, 99.78 μmol, 33.08% yield) according to the procedures described herein and the synthetic scheme, using 7-bromo-1-hydroxy-2,3,1-benzoxazaborinine (54.5 mg, 241.28 μmol, 0.8 eq), 2-(4,4-dimethyl-1-piperidyl)-3,6-dimethyl-8-[(1R)-1-[2-(4,4,5,5-tetramethyl-1,3,2-dioxaborolan-2-yl)anilino]ethyl]chromen-4-one (200 mg, 301.60 mol, 1 eq). ¹H NMR (DMSO-d₆, 400 MHz) δ 9.40 (s, 1H), 8.68 (s, 1H), 8.15 (s, 1H), 7.92-7.90 (m, 1H), 7.82-7.80 (m, 1H), 7.59 (s, 1H), 7.46 (s, 1H), 7.08-7.03 (m, 2H), 6.72-6.69 (m, 1H), 6.54 (d, J=8.0 Hz, 1H), 5.02-5.00 (m, 1H), 4.95-4.91 (m, 1H), 3.28-3.24 (m, 4H), 2.31 (s, 3H), 1.89 (s, 3H), 1.45-1.39 (m, 7H), 0.95 (s, 6H). MS (ESI): mass calcd. For C33H36BN3O4 549.48, m/z found 550.2 [M+H]⁺. HPLC: 97.03% (220 nm), 98.39% (254 nm). Chiral purity: 97.38% ee.

### Example 31

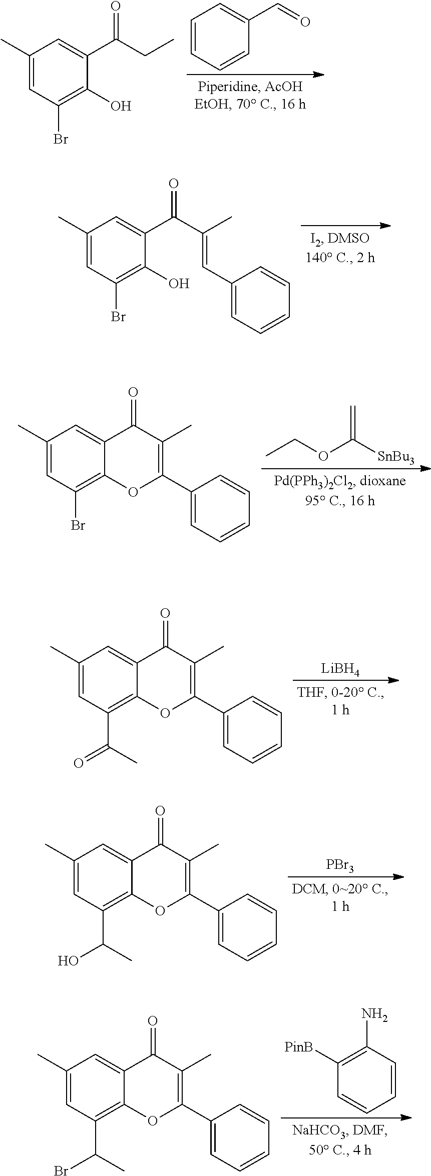

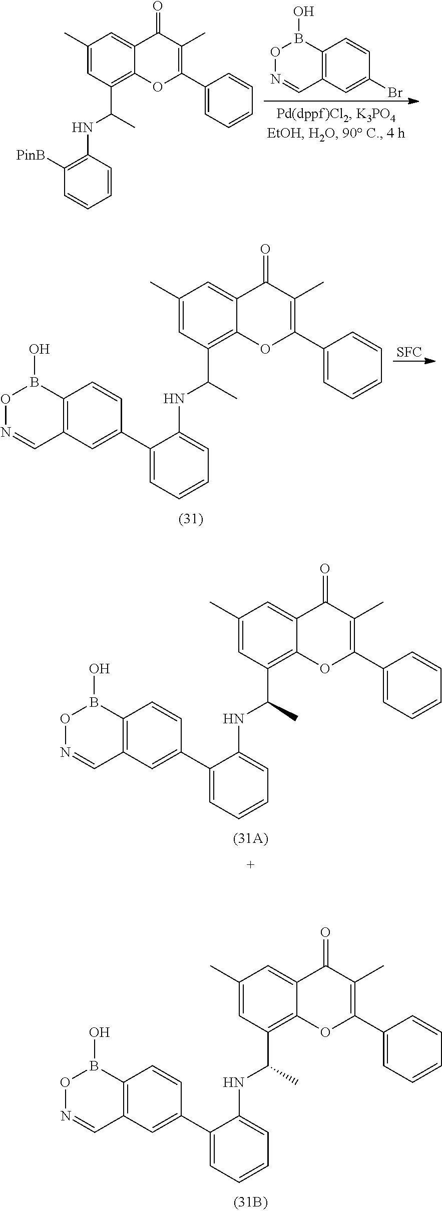

### Preparation of (E)-1-(3-bromo-2-hydroxy-5-methyl-phenyl)-2-methyl-3-phenyl-prop-2-en-1-one

To a solution of 1-(3-bromo-2-hydroxy-5-methyl-phenyl)propan-1-one (14.00 g, 57.6 mmol, 1 eq), benzaldehyde (6.72 g, 63.3 mmol, 6.40 mL, 1.1 eq) in EtOH (16 mL) was added piperidine (12.3 g, 144 mmol, 14.2 mL, 2.5 eq) and AcOH (7.61 g, 126 mmol, 7.25 mL, 2.2 eq) in turns at 20° C., the mixture was heated to 70° C. and stirred at 70° C. for 16 h. The reaction mixture was poured into ice-water (w/w=1/1) (300 mL) and extracted with DCM (200 mL×3). The combined organic phase was washed with brine (100 mL), dried over anhydrous Na₂SO₄, filtered and concentrated in vacuo to give the title compound (20 g, crude) was obtained as yellow oil, which was used for next step directly. ¹H NMR (CDCl3, 400 MHz) δ7.67-7.41 (m, 7H), 5.13 (d, J=11.6 Hz, 1H), 2.32 (s, 3H), 1.06 (d, J=7.2 Hz, 1H).

### Preparation of 8-bromo-3,6-dimethyl-2-phenyl-chromen-4-one

To a solution of (E)-1-(3-bromo-2-hydroxy-5-methyl-phenyl)-2-methyl-3-phenyl-prop-2-en-1-one (15.00 g, 45.3 mmol, 1 eq) in DMSO (100 mL) was added I₂ (1.15 g, 4.53 mmol, 912 μL, 0.1 eq) portion-wise at 20° C., then the mixture was heated to 140° C. and stirred at 140° C. for 2 h. The reaction mixture was cooled to 25° C., poured into ice-water (w/w=1/1) (300 mL) and stirred for 2 min. The resulting suspension was filtered and the filter cake was washed with water (100 mL). The filter cake was triturated with H₂O (200 mL) and then dried in vacuo to give the title compound (14.00 g, 42.53 mmol, 93.90% yield) as yellow solid. ¹H NMR (CDCl₃, 400 MHz) δ 8.01-8.00 (m, 1H), 7.77-7.73 (m, 3H), 7.56-7.54 (m, 3H), 2.47 (s, 3H), 2.23 (s, 3H).

### Preparation of 8-acetyl-3,6-dimethyl-2-phenyl-chromen-4-one

To a solution of 8-bromo-3,6-dimethyl-2-phenyl-chromen-4-one (14.0 g, 42.5 mmol, 1 eq) and tributyl(1-ethoxyvinyl)stannane (25.7 g, 71.1 mmol, 1.67 eq) in dioxane (200 mL) was added Pd(PPh₃)₂Cl₂ (1.49 g, 2.13 mmol, 0.05 eq) in one portion at 20° C., the mixture was degassed and purged with N₂ for 3 times and stirred at 95° C. for 16 h under N₂ atmosphere. The reaction mixture was cooled to 20° C., treated with HCl (30 mL, 2 N) and stirred at 50° C. for 0.5 h. To the mixture was added sat.aq. KF (200 mL), the mixture was stirred for 0.5 h. Then the resulting suspension was filtered, the filter cake was washed with EtOAc (250 mL×3) and the filtrate was extracted with EtOAc (250 mL×3). The combined organic phase was washed with brine (250 mL), dried over Na₂SO₄, filtered and concentrated under reduced pressure. The crude product was triturated with MTBE (50 mL) at 20° C. for 30 min to give the title compound (12.00 g, 41.0 mmol, 96.52% yield) as a yellow solid. ¹H NMR (CDCl₃, 400 MHz) δ 8.25 (s, 1H), 7.95 (s, 1H), 7.68-7.66 (m, 2H), 7.55-7.54 (m, 3H), 2.71 (s, 3H), 2.50 (s, 3H), 2.19 (s, 3H).

### Preparation of 8-(1-hydroxyethyl)-3,6-dimethyl-2-phenyl-chromen-4-one

To a solution of 8-acetyl-3,6-dimethyl-2-phenyl-chromen-4-one (2.5 g, 8.55 mmol, 1 eq) in THF (30 mL) was added LiBH₄ (2 M in THF, 5.13 mL, 1.2 eq) dropwise at 0° C., the mixture was stirred at 20° C. for 1 h. The mixture was poured into ice-water (w/w=1/1) (50 mL) and stirred for 10 min. The aqueous phase was extracted with EtOAc (30 mL×3). The combined organic phases were washed with brine (30 mL), dried with anhydrous Na₂SO₄, filtered and concentrated in vacuo to give the title compound (2.5 g, 8.49 mmol, 99.32% yield) as a yellow solid, which was used directly for next step. ¹H NMR (DMSO-d₆, 400 MHz) δ 7.95 (s, 1H), 7.95-7.63 (m, 3H), 7.56-7.53 (m, 3H), 5.48-5.42 (m, 1H), 2.46 (s, 3H), 2.18 (s, 3H), 1.58 (d, J=6.8 Hz, 3H).

### Preparation of 8-(1-bromoethyl)-3,6-dimethyl-2-phenyl-chromen-4-one

To s solution of 8-(1-hydroxyethyl)-3,6-dimethyl-2-phenyl-chromen-4-one (2.50 g, 8.49 mmol, 1 eq) in DCM (30 mL) was added PBr₃ (2.76 g, 10.19 mmol, 1.2 eq) at 0° C. in portions, the mixture was stirred at 20° C. for 1 h. The mixture was poured into ice-water (w/w=1/1) (100 mL) and extracted with DCM (50 mL×3). The combined organic phases were washed with brine (20 mL), dried with anhydrous Na₂SO₄, filtered and concentrated in vacuo. The residue was purified by flash silica gel chromatography (ISCO®; 40 g SepaFlash® Silica Flash Column, Eluent of 0-50% Ethyl acetate/Petroleum ether gradient @50 m/min) to give the title compound (1.50 g, 4.20 mmol, 49.44% yield) as a yellow solid. ¹H NMR (DMSO-d₆, 400 MHz) δ 8.03 (s, 1H), 8.02-7.75 (m, 2H), 7.66 (d, J=2.0 Hz, 1H), 7.56-7.55 (m, 3H), 5.78-5.73 (m, 1H), 2.49 (s, 3H), 2.21 (s, 3H), 2.12 (d, J=7.2 Hz, 3H).

### Preparation of 3,6-dimethyl-2-phenyl-8-[1-[2-(4,4,5,5-tetramethyl-1,3,2-dioxaborolan-2-yl)anilino]ethyl]chromen-4-one

A mixture of 8-(1-bromoethyl)-3,6-dimethyl-2-phenyl-chromen-4-one (500 mg, 1.40 mmol, 1 eq), 2-(4,4,5,5-tetramethyl-1,3,2-dioxaborolan-2-yl)aniline (399 mg, 1.82 mmol, 1.3 eq) and NaHCO₃ (177 mg, 2.10 mmol, 1.5 eq) in DMF (8 mL) was stirred at 50° C. for 4 h. The reaction mixture was poured into ice-water (w/w=1/1) (50 mL) and stirred for 2 min. The resulting suspension was filtered and the filter cake was dried in vacuo to give the title compound (500 mg, 1.01 mmol, 72.11% yield) as white solid, which was used directly for next step without further purification.

### Preparation of 8-(1-((2-(1-hydroxy-1H-benzo[d][1,2,6]oxazaborinin-6-yl)phenyl)amino)ethyl)-3,6-dimethyl-2-phenyl-4H-chromen-4-one

A mixture of 3,6-dimethyl-2-phenyl-8-[1-[2-(4,4,5,5-tetramethyl-1,3,2-dioxaborolan-2-yl)anil ino]ethyl]chromen-4-one (500 mg, 1.01 mmol, 1 eq), 6-bromo-1-hydroxy-2,3,1-benzoxazaborinine (296 mg, 1.31 mmol, 1.3 eq), Pd(dppf)Cl₂ (74 mg, 100 μmol, 0.1 eq) and K₃PO₄ (428 mg, 2.01 mmol, 2 eq) in EtOH (5 mL) and H₂O (1 mL) was de-gassed and then heated to 90° C. for 4 h under N₂. The reaction mixture was cooled to 25° C., filtered and the filtrate was directly purified by prep-HPLC (column: Phenomenex Gemini-□X 150×30 mm×5 um; mobile phase: [H2O (0.1% TFA)-AC□]; gradient:45%-70% B over 30.0 min) to give the title compound (150 mg, 247.43 μmol, 24.57% yield) as a white solid. ¹H NMR (DMSO-d₆, 400 MHz) δ 9.45-9.38 (m, 1H), 8.62 (s, 1H), 8.05 (s, 1H), 7.78-7.76 (m, 3H), 7.73-7.65 (m, 2H), 7.65-7.62 (m, 1H), 7.54-7.53 (m, 3H), 7.03-7.01 (m, 2H), 6.69-6.69 (m, 1H), 6.56-6.53 (m, 1H), 5.08-5.04 (m, 1H), 4.97-4.95 (m, 1H), 2.38 (s, 3H), 2.05 (s, 3H), 1.45 (d, J=6.4 Hz, 3H). MS (ESI): mass calcd. For C₃₂H₂₇BN₂O₄ 514.39, m/z found 515.3 [M+H]⁺. HPLC: 98.17% (220 nm), 98.96% (254 nm).

### Preparation of (R)-8-(1-((2-(1-hydroxy-1H-benzo[d][1,2,6]oxazaborinin-6-yl)phenyl)amino)ethyl)-3,6-dimethyl-2-phenyl-4H-chromen-4-one and (S)-8-(1-((2-(1-hydroxy-1H-benzo[d][1,2,6]oxazaborinin-6-yl)phenyl)amino)ethyl)-3,6-dimethyl-2-phenyl-4H-chromen-4-one

8-(1-((2-(1-hydroxy-1H-benzo[d][1,2,6]oxazaborinin-6-yl)phenyl)amino)ethyl)-3,6-dimethyl-2-phenyl-4H-chromen-4-one (180 mg, 349 μmol, 1 eq) was purified by SFC (column: DAICEL CHIRALCEL OD (250 mm×30 mm, 10 um); mobile phase: [CO₂-EtOH (0.1□H3H2O)]; B %:50%, isocratic elution mode) to give the title compound, Isomer 1 (64.5 mg, 125.39 μmol, 35.83% yield) as a white solid and the title compound, Isomer 2 (64.4 mg, 125.20 μmol, 35.78% yield) as a white solid. Isomer 1: ¹H NMR (DMSO-d₆, 400 MHz) δ 9.42-9.36 (m, 1H), 8.61 (s, 1H), 8.06-8.05 (m, 1H), 7.78-7.74 (m, 3H), 7.67-7.66 (m, 2H), 7.54-7.54 (m, 1H), 7.54-7.53 (m, 3H), 7.06-7.01 (m, 2H), 6.69-6.69 (m, 1H), 6.55-6.55 (m, 1H), 5.05-4.94 (m, 2H), 2.37 (s, 3H), 2.05 (s, 3H), 1.45 (d, J=6.4 Hz, 3H). MS (ESI): mass calcd. For C₃₂H₂₇BN₂O₄ 514.39, m/z found 515.2 [M+H]⁺. HPLC: 98.64% (220 nm), 98.93% (254 nm). Chiral purity: 100 ee %. Isomer 2: ¹H NMR (DMSO-d₆, 400 MHz) δ 9.41-9.36 (m, 1H), 8.61 (s, 1H), 8.06-8.05 (m, 1H), 7.78-7.74 (m, 3H), 7.67-7.65 (m, 2H), 7.54-7.54 (m, 1H), 7.54-7.53 (m, 3H), 7.06-7.01 (m, 2H), 6.69-6.69 (m, 1H), 6.55-6.55 (m, 1H), 5.05-5.03 (m, 1H), 4.98-4.94 (m, 1H), 2.38 (s, 3H), 2.05 (s, 3H), 1.45 (d, J=6.4 Hz, 3H). MS (ESI): mass calcd. For C₃₂H₂₇BN₂O₄ 514.39, m/z found 515.2 [M+H]⁺. HPLC: 99.74% (220 nm), 99.72% (254 nm). Chiral purity: 99.18 ee %.

### Example 32

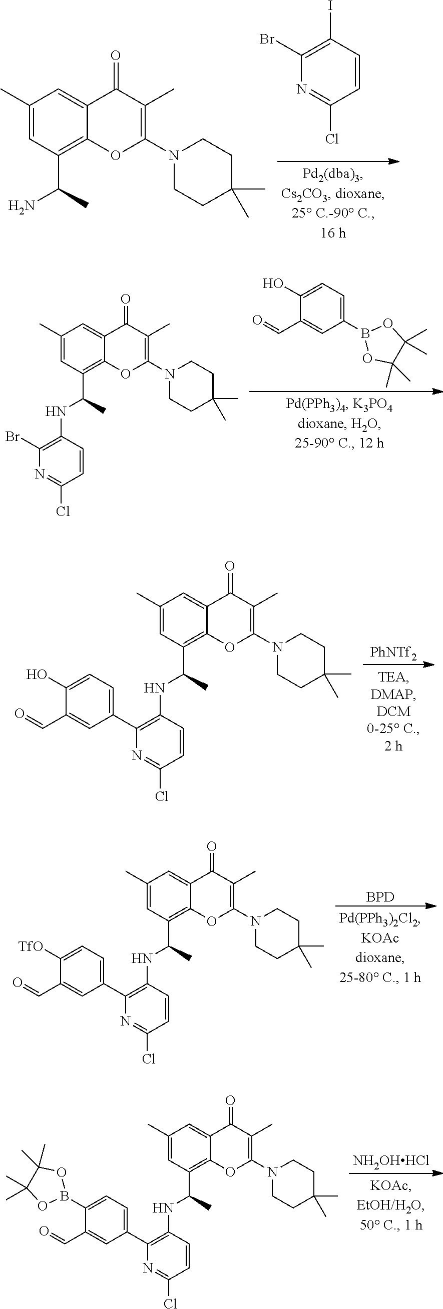

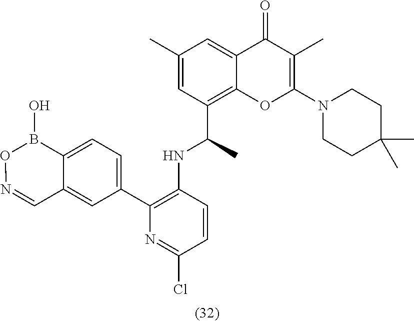 [SMILES: Cc1cc([C@@H](C)Nc2ccc(Cl)nc2-c2ccc3c(c2)C=NOB3O)c2oc(N3CCC(C)(C)CC3)c(C)c(=O)c2c1]

### Preparation of 8-[(1R)-1-[(2-bromo-6-chloro-3-pyridyl)amino]ethyl]-2-(4,4-dimethyl-1-piperidyl)-3,6-dimethyl-chromen-4-one

To a solution of (R)-8-(1-aminoethyl)-2-(4,4-dimethylpiperidin-1-yl)-3,6-dimethyl-4H-chromen-4-one (3.00 g, 9.13 mmol, 1 eq) and 2-bromo-6-chloro-3-iodo-pyridine (5.82 g, 18.27 mmol, 2 eq) and Cs₂CO₃ (5.95 g, 18.27 mmol, 2 eq) in dioxane (40 mL) were added Pd₂(dba)₃ (836.4 mg, 913.39 mol, 0.1 eq) and (5-diphenylphosphanyl-9,9-dimethyl-xanthen-4-yl)-diphenyl-phosphane (528.5 mg, 913.39 mol, 0.1 eq) in turns at 25° C., the reaction mixture was heated to 90° C. and stirred at 90° C. for 16 h. The reaction mixture was cooled to 25° C. and filtered. The filtrate was concentrated under reduced pressure and the residue was purified by column chromatography (SiO₂, Petroleum ether/Ethyl acetate=3/1 to 1/1).to give the title compound (4.00 g, 7.71 mmol, 84.40% yield) as yellow oil. ¹H NMR (CDCl₃, 400 MHz) δ 7.94 (s, 1H), 8.17 (d, J=8.0 Hz, 1H), 7.30 (s, 1H), 6.99 (d, J=8.4 Hz, 1H), 6.49 (d, J=8.8 Hz, 1H), 4.93 (t, J=5.2 Hz, 1H), 4.83 (d, J=4.8 Hz, 1H), 3.49-3.39 (m, 4H), 2.39 (s, 3H), 2.10 (s, 3H), 1.71 (d, J=6.4 Hz, 3H), 1.55 (t, J=6.0 Hz, 4H), 1.28 (s, 6H).

### Preparation of 5-[6-chloro-3-[[(1R)-1-[2-(4,4-dimethyl-1-piperidyl)-3,6-dimethyl-4-oxo-chromen-8-yl]ethyl]amino]-2-pyridyl]-2-hydroxy-benzaldehyde

To a solution of (R)-8-(1-((2-bromo-6-chloropyridin-3-yl)amino)ethyl)-2-(4,4-dimethylpiperidin-1-yl)-3,6-dimethyl-4H-chromen-4-one (1.90 g, 3.66 mmol, 1 eq) and 2-hydroxy-5-(4,4,5,5-tetramethyl-1,3,2-dioxaborolan-2-yl)benzaldehyde (908.4 mg, 3.66 mmol, 1 eq) in dioxane (20 mL)/H₂O (5 mL) were added Pd(PPh₃)₄ (423.1 mg, 366.18 μmol, 0.1 eq) and K₃PO₄ (1.55 g, 7.32 mmol, 2 eq) in turns at 25° C., the mixture was heated to 90° C. and stirred at 90° C. for 12 h. 2 parallel reactions were combined for work up. The reaction mixture was filtered and the filtrate was concentrated under reduced pressure to give a residue. The residue was purified by column chromatography (SiO2, Petroleum ether/Ethyl acetate=3/1 to 2/1) to give the title compound (4.00 g, 7.14 mmol, 97.52% yield) as yellow oil. ¹H NMR (CDCl₃, 400 MHz) δ 11.21 (s, 1H), 10.02 (s, 1H), 8.05-7.90 (m, 3H), 7.30-7.27 (m, 1H), 7.19 (d, J=8.8 Hz, 1H), 7.04 (d, J=8.4 Hz, 1H), 6.69 (d, J=8.8 Hz, 1H), 4.93 (d, J=6.8 Hz, 1H), 3.49-3.39 (m, 4H), 2.42 (s, 3H), 2.11 (s, 3H), 1.71 (d, J=6.8 Hz, 3H), 1.54 (t, J=5.6 Hz, 4H), 1.06 (s, 6H).

### Preparation of (R)-4-(6-chloro-3-((1-(2-(4,4-dimethylpiperidin-1-yl)-3,6-dimethyl-4-oxo-4H-chromen-8-yl)ethyl)amino)pyridin-2-yl)-2-formylphenyl trifluoromethanesulfonate

To a solution of 5-[6-chloro-3-[[(1R)-1-[2-(4,4-dimethyl-1-piperidyl)-3,6-dimethyl-4-oxo-chromen-8-yl]ethyl]amino]-2-pyridyl]-2-hydroxy-benzaldehyde (3.80 g, 6.78 mmol, 1 eq) and [N-(trifluoromethylsulfonyloxy)anilino]trifluoromethanesulfonate (2.64 g, 6.78 mmol, 1 eq) in DCM (50 mL) were added TEA (1.37 g, 13.57 mmol, 1.89 mL, 2 eq) and DMAP (82.89 mg, 678.5 μmol, 0.1 eq) in turns at 0° C., the reaction mixture was stirred at 0-25° C. for 2 h. The reaction mixture was quenched by adding water (50 mL) and extracted with EtOAc (50 mL×3). The combined organic layers were washed with brine (50 mL×2), dried over Na₂SO₄, filtered and concentrated under reduced pressure to give a residue. The residue was purified by column chromatography (SiO₂, Petroleum ether/Ethyl acetate=3/1 to 1/1) to give the title compound (4.00 g, 5.78 mmol, 85.18% yield) as yellow oil, which was used directly for next step without further purification.

### Preparation of (R)-5-(6-chloro-3-((1-(2-(4,4-dimethylpiperidin-1-yl)-3,6-dimethyl-4-oxo-4H-chromen-8-yl)ethyl)amino)pyridin-2-yl)-2-(4,4,5,5-tetramethyl-1,3,2-dioxaborolan-2-yl)benzaldehyde

To a solution of (R)-4-(6-chloro-3-((1-(2-(4,4-dimethylpiperidin-1-yl)-3,6-dimethyl-4-oxo-4H-chromen-8-yl)ethyl)amino)pyridin-2-yl)-2-formylphenyl trifluoromethanesulfonate (1.00 g, 1.44 mmol, 1 eq), B₂Pin₂ (1.47 g, 5.78 mmol, 4 eq) and KOAc (425.4 mg, 4.33 mmol, 3 eq) in dioxane (10 mL) was added Pd(PPh₃)₂Cl₂ (101.4 mg, 144.48 μmol, 0.1 eq) in one portion at 25° C., the mixture was heated to 80° C. and stirred at 80° C. for 1 h. 3 parallel reactions were combined for work up. The reaction mixture was cooled to 20° C., filtered and the filtrate was concentrated under reduced pressure to give a residue.

The residue was purified by column chromatography (SiO₂, Petroleum ether/Ethyl acetate=3/1 to 1/1) to give the title compound (2.50 g, 3.73 mmol, 86.08% yield) as a yellow solid. ¹H NMR (DMSO-d₆, 400 MHz) δ 10.70 (s, 1H), 8.42 (s, 1H), 8.13-8.11 (m, 1H), 8.04-8.01 (m, 1H), 7.10 (d, J=8.8 Hz, 1H), 6.73 (d, J=8.4 Hz, 1H), 5.00-4.96 (m, 1H), 4.54-4.51 (m, 1H), 3.46-3.45 (m, 4H), 2.47 (s, 3H), 2.14 (s, 3H), 1.62-1.57 (m, 7H), 1.35 (s, 12H), 1.12 (s, 6H)

### Preparation of (R)-8-(1-((6-chloro-2-(1-hydroxy-1H-benzo[d][1,2,6]oxazaborinin-6-yl)pyridin-3-yl)amino)ethyl)-2-(4,4-dimethylpiperidin-1-yl)-3,6-dimethyl-4H-chromen-4-one

To a solution of (R)-5-(6-chloro-3-((1-(2-(4,4-dimethylpiperidin-1-yl)-3,6-dimethyl-4-oxo-4H-chromen-8-yl)ethyl)amino)pyridin-2-yl)-2-(4,4,5,5-tetramethyl-1,3,2-dioxaborolan-2-yl)benzaldehyde (1.40 g, 2.09 mmol, 1 eq) and NH₂OH·HCl (145.20 mg, 2.09 mmol, 1 eq) in EtOH (10 mL)/H₂O (10 mL) was added KOAc (205.06 mg, 2.09 mmol, 1 eq) in one portion at 25° C., the reaction mixture was heated to 50° C. and stirred at 50° C. for 1 h. The reaction mixture was cooled to 25° C. and filtered directly. The crude product (700 mg) was triturated with MeCN (10 mL) at 25° C. for 30 min to give the title compound (282.1 mg) as a white solid. ¹H NMR (DMSO-d₆, 400 MHz) δ 9.48 (s, 1H), 8.72 (s, 1H), 8.17 (d, J=8.0 Hz, 1H), 8.10-8.02 (m, 2H), 7.63 (s, 1H), 7.47 (s, 1H), 7.21 (d, J=8.4 Hz, 1H), 7.00 (d, J=8.8 Hz, 1H), 5.85 (d, J=6.8 Hz, 1H), 4.93 (t, J=6.8 Hz, 1H), 3.34-3.20 (m, 4H), 2.32 (s, 3H), 1.91 (s, 3H), 1.52 (d, J=6.8 Hz, 3H), 1.41 (t, J=5.6 Hz, 4H), 0.97 (s, 6H). MS (ESI): mass calcd. For C₃₀H₃₀BClN₄O₄ 556.20, m/z found 557.1 [M+H]⁺. HPLC: 98.01% (220 nm), 98.15% (254 nm). Chiral purity: 99.48% ee.

### Example 33

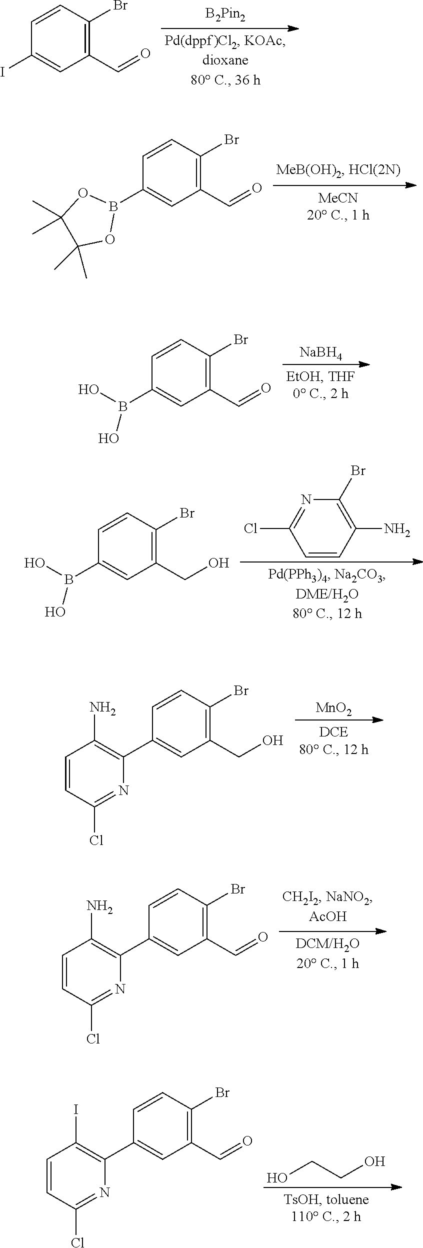

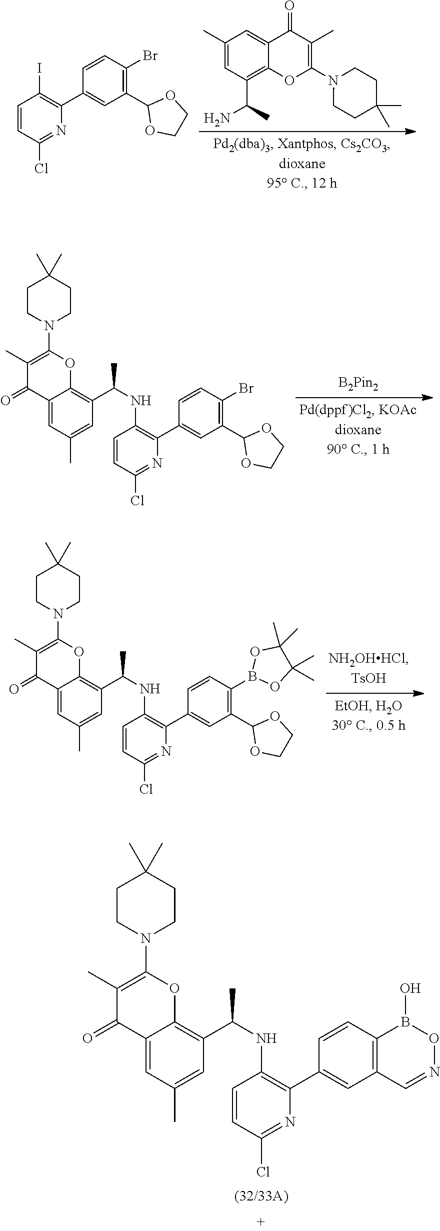

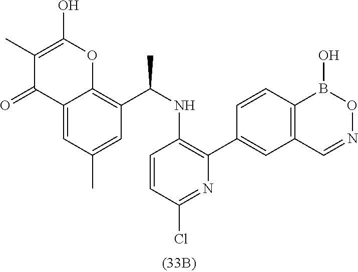 [SMILES: Cc1cc([C@@H](C)Nc2ccc(Cl)nc2-c2ccc3c(c2)C=NOB3O)c2oc(O)c(C)c(=O)c2c1]

### Preparation of 2-bromo-5-(4,4,5,5-tetramethyl-1,3,2-dioxaborolan-2-yl)benzaldehyde

A mixture of 2-bromo-5-iodo-benzaldehyde (3.00 g, 9.65 mmol, 1.00 eq), 4,4,5,5-tetramethyl-2-(4,4,5,5-tetramethyl-1,3,2-dioxaborolan-2-yl)-1,3,2-dioxaborolane (6.13 g, 24.12 mmol, 2.50 eq), Pd(dppf)Cl₂ (706 mg, 964.90 μmol, 0.10 eq) and KOAc (1.89 g, 19.30 mmol, 2.00 eq) in dioxane (30 mL) was degassed and purged with N₂ for 3 times at 20° C. Then the reaction mixture was heated to 80° C. and stirred at 80° C. for 36 h under N₂ atmosphere. The reaction mixture was cooled to 25° C., filtered through a pad of Clite and the filter cake was washed with EtOAc (120 mL). The filtrate was concentrated under reduced pressure and the crude product was purified by flash silica gel chromatography (Biotage®; 120 g SepaFlash® Silica Flash Column, Eluent of 0~4% Petroleum ether/Ethyl acetate gradient@80 mL/min) to give the title compound (12.00 g, crude) as yellow oil. ¹H NMR (DMSO-d₆, 400 MHz) δ 10.23 (s, 1H), 8.09 (s, 1H), 7.81-7.80 (m, 2H), 1.31 (s, 12H).

### Preparation of (4-bromo-3-formyl-phenyl)boronic acid

To a solution of 2-bromo-5-(4,4,5,5-tetramethyl-1,3,2-dioxaborolan-2-yl)benzaldehyde (12.00 g, 38.59 mmol, 1.00 eq) in MeCN (100 mL)/HCl (2 N, 20 mL) was added MeB(OH)₂ (23.10 g, 385.88 mmol, 10.00 eq) in portions at 20° C. The reaction mixture was stirred at 20° C. for 1 h. The reaction mixture was filtered, and the filtrate was concentrated under reduced pressure. The crude product was triturated with MeCN (25 mL) at 20° C. for 30 mins to give the title compound (4.50 g, 19.66 mmol, 50.96% yield) as a white solid. ¹H NMR (DMSO-d₆, 400 MHz) δ 10.25 (s, 1H), 8.42 (s, 2H), 8.28 (d, J=1.6 Hz, 1H), 7.95 (dd, J=8.0 Hz, 1.6 Hz, 1H), 7.78 (d, J=8.0 Hz, 1H).

### Preparation of [4-bromo-3-(hydroxymethyl)phenyl]boronic acid

To a solution of (4-bromo-3-formyl-phenyl)boronic acid (2.90 g, 12.67 mmol, 1.00 eq) in EtOH (15 mL)/THF (15 mL) was added NaBH₄ (259 mg, 6.84 mmol, 0.54 eq) in portions at 0° C., the reaction mixture was stirred at 0° C. for 2 h. The mixture was poured into ice water (50 mL), adjusted pH=5 with 2 N HCl and then extracted with EtOAc (30 mL×3). The combined organic phase was washed with brine (30 mL), dried over Na₂SO₄, filtered and concentrated under reduced pressure to give the title compound (2.60 g, 11.26 mmol, 88.87% yield) as a pale yellow solid. ¹H NMR (DMSO-d₆, 400 MHz) δ 8.13 (s, 2H), 7.96 (s, 1H), 7.59 (d, J=8.0 Hz, 1H), 7.53 (d, J=8.0 Hz, 1H), 4.51 (s, 2H).

### Preparation of [5-(3-amino-6-chloro-2-pyridyl)-2-bromo-phenyl]methanol

A mixture of [4-bromo-3-(hydroxymethyl)phenyl]boronic acid (2.60 g, 11.26 mmol, 1.46 eq), 2-bromo-6-chloro-pyridin-3-amine (1.60 g, 7.71 mmol, 1.00 eq), Pd(PPh₃)₄ (891.23 mg, 771.25 μmol, 0.10 eq) and Na₂CO₃ (1.63 g, 15.42 mmol, 2.00 eq) in H₂O (4 mL)/dioxane (16 mL) was degassed and purged with N₂ for 3 times at 20° C., and then the reaction mixture was heated 80° C. and stirred at 80° C. for 12 h under N₂ atmosphere. The mixture was cooled to 20° C., diluted with water (40 mL) and extracted with EtOAc (15 mL×3). The combined organic phase was washed with brine (15 mL), dried over Na₂SO₄, filtered and concentrated under reduced pressure. The crude product was purified by flash silica gel chromatography (Biotage®; 20 g SepaFlash® Silica Flash Column, Eluent of 0~35% Petroleum ether/Ethyl acetate gradient@30 mL/min). The eluent was removed under reduced pressure to give the title compound (1.10 g, 3.51 mmol, 45.48% yield) as a yellow solid. ¹H NMR (DMSO-d₆, 400 MHz) δ 7.80 (s, 1H), 7.65 (d, J=8.4 Hz, 1H), 7.48 (dd, J=8.0 Hz, 2.0 Hz, 1H), 7.22 (d, J=8.4 Hz, 1H), 7.17 (d, J=8.4 Hz, 1H), 5.48 (t, J=5.6 Hz, 1H), 5.37 (s, 2H), 4.57 (d, J=5.6 Hz, 2H).

### Preparation of 5-(3-amino-6-chloro-2-pyridyl)-2-bromo-benzaldehyde

To a solution of [5-(3-amino-6-chloro-2-pyridyl)-2-bromo-phenyl]methanol (1.10 g, 3.51 mmol, 1.00 eq) in DCE (2 mL) was added MnO₂ (3.05 g, 35.08 mmol, 10.00 eq) in portions at 25° C. The reaction mixture was heated to 80° C. and stirred at 80° C. for 12 h. The mixture was cooled to 25° C., and then filtered through a pad of Clite. The filter cake was washed with DCM (50 mL) and the filtrate was concentrated under reduced pressure to give the title compound (0.95 g, crude) as a brown solid, which was used directly for next step without further purification. ¹H NMR (DMSO-d₆, 400 MHz) δ 10.26 (s, 1H), 8.13 (s, 1H), 7.88-7.86 (m, 2H), 7.26 (d, J=8.4 Hz, 1H), 7.20 (d, J=8.8 Hz, 1H), 5.52 (s, 2H).

### Preparation of 2-bromo-5-(6-chloro-3-iodo-2-pyridyl)benzaldehyde

To a solution of 5-(3-amino-6-chloro-2-pyridyl)-2-bromo-benzaldehyde (0.93 g, 2.98 mmol, 1.00 eq), NaNO₂ (1.03 g, 14.92 mmol, 5.00 eq) in H₂O (3 mL) and DCM (6 mL) was added CH₂I₂ (1.60 g, 5.97 mmol, 481.61 μL, 2.00 eq) dropwise at 20° C. After addition, the reaction mixture was stirred for 5 min and then AcOH (3.59 g, 59.70 mmol, 3.42 mL, 20.00 eq) was added dropwise at 20° C. The resulting reaction mixture was stirred at 20° C. for 1 h. To the mixture was added water (20 mL) at 20° C. and then the mixture was extracted with DCM (10 mL×3). The combined organic layers were washed with brine (10 mL), dried over Na₂SO₄, filtered and concentrated under reduced pressure. The crude product was purified by flash silica gel chromatography (Biotage®; 20 g SepaFlash® Silica Flash Column, Eluent of 0~15% Petroleum ether/Ethyl acetate gradient@55 mL/min). The eluent was removed under reduced pressure to give the title compound (0.45 g, 1.07 mmol, 35.69% yield) as a yellow solid. ¹H NMR (DMSO-d₆, 400 MHz) δ 10.27 (s, 1H), 8.46 (d, J=8.4 Hz, 1H), 8.04 (d, J=2.4 Hz, 1H), 7.96 (d, J=8.4 Hz, 1H), 7.85 (dd, J=8.4 Hz, 2.0 Hz, 1H), 7.37 (d, J=8.4 Hz, 1H).

### Preparation of 2-[4-bromo-3-(1,3-dioxolan-2-yl)phenyl]-6-chloro-3-iodo-pyridine

To a solution of 2-bromo-5-(6-chloro-3-iodo-2-pyridyl)benzaldehyde (0.55 g, 1.30 mmol, 1.00 eq) in toluene (5 mL) and ethylene glycol (242.43 mg, 3.91 mmol, 217.81 μL, 3.00 eq) was added TsOH (56.05 mg, 325.49 μmol, 0.25 eq) in portions at 20° C. The reaction mixture was heated to 110° C. and stirred at 110° C. for 2 h. The mixture was cooled to rt., diluted with water (15 mL) and extracted with EtOAc (5 mL×3). The combined organic layers were washed with brine (5 mL), dried over Na₂SO₄, filtered and concentrated under reduced pressure. The crude product was purified by flash silica gel chromatography (Biotage®; 12 g SepaFlash® Silica Flash Column, Eluent of 0~27% Petroleum ether/Ethyl acetate gradient@35 mL/min) to give the title compound (0.40 g, 857.46 μmol, 65.86% yield) as a white solid. ¹H NMR (DMSO-d₆, 400 MHz) δ 8.43 (d, J=8.4 Hz, 1H), 7.77 (d, J=8.4 Hz, 1H), 7.73 (d, J=2.4 Hz, 1H), 7.55 (dd, J=8.0 Hz, 2.0 Hz, 1H), 7.34 (d, J=8.4 Hz, 1H), 6.02 (s, 1H), 4.12-4.09 (m, 2H), 4.02-3.99 (m, 2H).

### Preparation of 8-[(1R)-1-[[2-[4-bromo-3-(1,3-dioxolan-2-yl)phenyl]-6-chloro-3-pyridyl]amino]ethyl]-2-(4,4-dimethyl-1-piperidyl)-3,6-dimethyl-chromen-4-one

The title compound was prepared as a yellow solid (0.42 g, 629.66 μmol, 82.72% yield) according to the similar procedure in Example 78, using 8-[(1R)-1-aminoethyl]-2-(4,4-dimethyl-1-piperidyl)-3,6-dimethyl-chromen-4-one (0.25 g, 761.16 μmol, 1.00 eq) and 2-[4-bromo-3-(1,3-dioxolan-2-yl)phenyl]-6-chloro-3-iodo-pyridine (355.08 mg, 761.16 μmol, 1.00 eq). ¹H NMR (DMSO-d₆, 400 MHz) δ 7.92 (d, J=2.0 Hz, 1H), 7.75 (d, J=8.0 Hz, 1H), 7.69 (dd, J=8.4 Hz, 2.0 Hz, 1H), 7.63 (s, 1H), 7.46 (d, J=2.0 Hz, 1H), 7.16 (d, J=8.8 Hz, 1H), 6.95 (d, J=8.8 Hz, 1H), 5.99 (s, 1H), 5.76 (d, J=6.8 Hz, 1H), 5.00-4.93 (m, 1H), 4.04-3.97 (m, 4H), 3.31-3.29 (m, 4H), 2.31 (s, 3H), 1.91 (s, 3H), 1.51 (d, J=6.8 Hz, 3H), 1.40 (t, J=5.6 Hz, 4H), 0.96 (s, 6H).

### Preparation of 8-[(1R)-1-[[6-chloro-2-[3-(1,3-dioxolan-2-yl)-4-(4,4,5,5-tetramethyl-1,3,2-dioxaborolan-2-yl)phenyl]-3-pyridyl]amino]ethyl]-2-(4,4-dimethyl-1-piperidyl)-3,6-dimethyl-chromen-4-one

The title compound was prepared as a yellow solid (0.35 g, 490.13 μmol, 88.36% yield) according to the procedures described herein and the synthetic scheme, using 8-[(1R)-1-[[2-[4-bromo-3-(1,3-dioxolan-2-yl)phenyl]-6-chloro-3-pyridyl]amino]ethyl]-2-(4,4-dimethyl-1-piperidyl)-3,6-dimethyl-chromen-4-one (0.37 g, 554.70 μmol, 1.00 eq) and B₂Pin₂ (423 mg, 1.66 mmol, 3.00 eq). ¹H NMR (DMSO-d₆, 400 MHz) δ 7.85 (s, 1H), 7.72-7.68 (m, 2H), 7.63 (s, 1H), 7.46 (d, J=2.0 Hz, 1H), 7.17 (d, J=8.8 Hz, 1H), 6.99 (d, J=8.8 Hz, 1H), 6.20 (s, 1H), 5.49 (d, J=6.8 Hz, 1H), 5.00-4.94 (m, 1H), 3.96-3.91 (m, 4H), 3.27-3.25 (m, 4H), 2.32 (s, 3H), 1.90 (s, 3H), 1.51 (d, J=6.4 Hz, 3H), 1.40 (t, J=5.6 Hz, 4H), 1.32 (s, 12H), 0.94 (s, 6H).

### Preparation of 8-[(1R)-1-[[6-chloro-2-(1-hydroxy-2,3,1-benzoxazaborinin-6-yl)-3-pyridyl]amino]ethyl]-2-(4,4-dimethyl-1-piperidyl)-3,6-dimethyl-chromen-4-one and by-product 8-[(1R)-1-[[6-chloro-2-(1-hydroxy-2,3,1-benzoxazaborinin-6-yl)-3-pyridyl]amino]ethyl]-2-hydroxy-3,6-dimethyl-chromen-4-one

To a solution of 8-[(1R)-1-[[6-chloro-2-[3-(1,3-dioxolan-2-yl)-4-(4,4,5,5-tetramethyl-1,3,2-dioxaborolan-2-yl)phenyl]-3-pyridyl]amino]ethyl]-2-(4,4-dimethyl-1-piperidyl)-3,6-dimethyl-chromen-4-one (0.20 g, 280.07 μmol, 1.00 eq) in EtOH (1.5 mL)/H₂O (0.3 mL) was added NH₂OH—HCl (38.92 mg, 560.15 μmol, 2.00 eq) and TsOH (4.82 mg, 28.01 μmol, 0.10 eq) in portions at 20° C. The mixture was heated to 30° C. and stirred at 30° C. for 0.5 h. The mixture was cooled to 0° C., adjusted pH=7 with sat.aq.

NaHCO₃ at 0° C. and filtered. The filtrate was purified by prep-HPLC (column: Phenomenex Gemini-NX 80×40 mm×3 um; mobile phase: [H₂O (10 mM NH₄HCO₃)-AC□]; gradient: 30%-60% B over 8.0 min) to give the title compound (15.70 mg, 26.10 μmol, 9.32% yield) as a white solid. ¹H NMR (DMSO-d₆, 400 MHz) δ 9.44 (s, 1H), 8.71 (s, 1H), 8.17 (d, J=8.0 Hz, 1H), 8.07-8.05 (m, 2H), 7.62 (s, 1H), 7.46 (d, J=2.0 Hz, 1H), 7.21 (d, J=8.8 Hz, 1H), 6.98 (d, J=8.8 Hz, 1H), 5.86 (d, J=7.2 Hz, 1H), 4.95-4.89 (m, 1H), 3.29 (t, J=4.8 Hz, 4H), 2.32 (s, 3H), 1.90 (s, 3H), 1.52 (d, J=6.4 Hz, 3H), 1.40 (t, J=5.6 Hz, 4H), 0.96 (s, 6H). MS (ESI): mass calcd. For C₃₂H₃₄BClN₄O₄ 584.24, m/z found 585.3 [M+H]⁺. HPLC: 97.24% (220 nm), 97.15% (254 nm). Chiral purity: 98.68% ee.

A by-product 8-[(1R)-1-[[6-chloro-2-(1-hydroxy-2,3,1-benzoxazaborinin-6-yl)-3-pyridyl]amino]ethyl]-2-hydroxy-3,6-dimethyl-chromen-4-one was also isolated (5.7 mg, 11.20 μmol, 4.00% yield) as a white solid. ¹H NMR (DMSO-d₆, 400 MHz) δ 9.47 (s, 1H), 8.77 (s, 1H), 8.20 (d, J=8.0 Hz, 1H), 8.07-8.05 (m, 2H), 7.55 (s, 1H), 7.33 (s, 1H), 7.18 (d, J=8.4 Hz, 1H), 6.89 (d, J=8.8 Hz, 1H), 5.80 (d, J=6.4 Hz, 1H), 4.91-4.84 (m, 1H), 2.29 (s, 3H), 1.94 (s, 3H), 1.45 (d, J=6.4 Hz, 3H). MS (ESI): mass calcd. For C₂₅H₂₁BClN₃O₅ 489.13, m/z found 490.2 [M+H]⁺. HPLC: 96.20% (220 nm), 96.42% (254 nm).

### Example 34

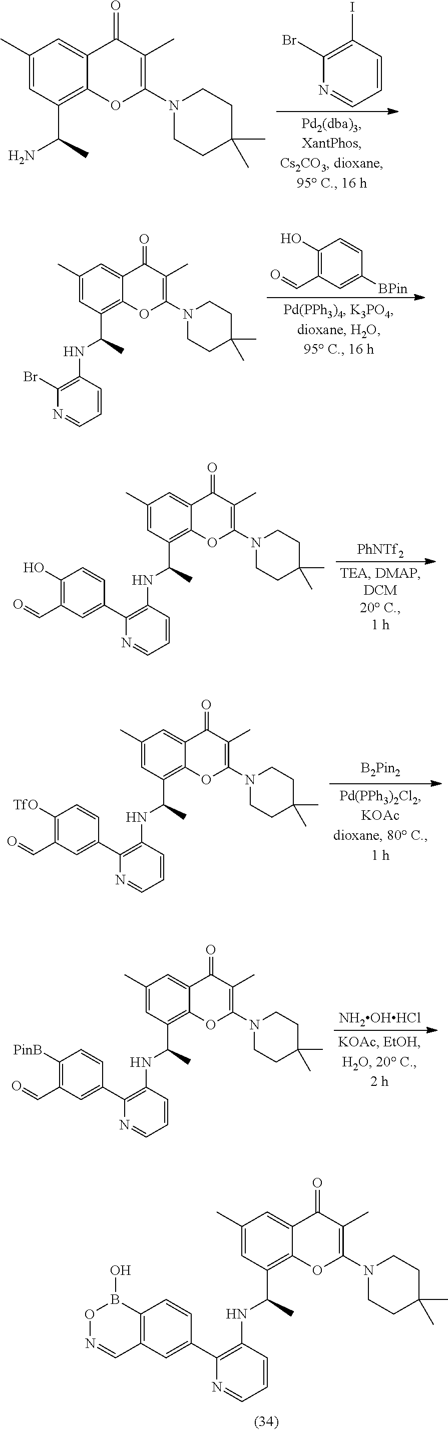

### Preparation of (R)-8-(1-((2-bromopyridin-3-yl)amino)ethyl)-2-(4,4-dimethylpiperidin-1-yl)-3,6-dimethyl-4H-chromen-4-one

To a solution of 2-bromo-3-iodopyridine (1.73 g, 6.09 mmol, 2 eq) and (R)-8-(1-aminoethyl)-2-(4,4-dimethylpiperidin-1-yl)-3,6-dimethyl-4H-chromen-4-one (1.00 g, 3.04 mmol, 1 eq) in dioxane (15 mL) was added Pd₂(dba)₃ (278 mg, 304 μmol, 0.1 eq), Xantphos (176 mg, 304 μmol, 0.1 eq) and Cs₂C₀₃ (1.98 g, 6.09 mmol, 2 eq) in turns at 20° C. The mixture was heated to 95° C. and stirred at 95° C. for 16 h under N₂. The reaction mixture was cooled to 25° C., filtered and the filtrate was concentrated under reduced pressure to give a residue. The residue was purified by flash silica gel chromatography (Biotage®; 20 g SepaFlash® Silica Flash Column, Eluent of 30~50% Ethyl acetate/Petroleum ether gradient@80 mL/min) to give the title compound (1.40 g, 2.89 mmol, 94.92% yield) as a yellow solid. ¹H NMR (CDCl3, 400 MHz) δ 7.89 (d, J=1.2 Hz, 1H), 7.69-7.68 (m, 1H), 7.32 (d, J=2.0 Hz, 1H), 6.97-6.93 (m, 1H), 6.51-6.49 (m, 1H), 4.97-4.91 (m, 1H), 4.84-4.83 (m, 1H), 3.40 (t, J=5.6 Hz, 4H), 2.37 (s, 3H), 2.06 (s, 3H), 1.69 (d, J=6.8 Hz, 3H), 1.53 (t, J=6.0 Hz, 4H), 1.04 (s, 6H).

### Preparation of (R)-5-(3-((1-(2-(4,4-dimethylpiperidin-1-yl)-3,6-dimethyl-4-oxo-4H-chromen-8-yl)ethyl)amino)pyridin-2-yl)-2-hydroxybenzaldehyde

A mixture of (R)-8-(1-((2-bromopyridin-3-yl)amino)ethyl)-2-(4,4-dimethylpiperidin-1-yl)-3,6-dimethyl-4H-chromen-4-one (1.00 g, 2.06 mmol, 1 eq), 2-hydroxy-5-(4,4,5,5-tetramethyl-1,3,2-dioxaborolan-2-yl)benzaldehyde (665 mg, 2.68 mmol, 1.3 eq), Pd(PPh₃)₄ (238 mg, 206 μmol, 0.1 eq) and K₃PO₄ (876 mg, 4.13 mmol, 2 eq) in dioxane (10 mL) and H₂O (2 mL) was degassed and purged with N₂ for 3 times at 20° C., and then the mixture was heated to 95° C. and stirred at 95° C. for 16 h under N₂ atmosphere. The reaction mixture was cooled to 25° C., filtered and the filtrate was concentrated under reduced pressure to give a residue. The residue was purified by flash silica gel chromatography (Biotage®; 20 g SepaFlash® Silica Flash Column, Eluent of 30~50% Ethyl acetate/Petroleum ether gradient@80 mL/min) to give the title compound (780 mg, crude) as a yellow solid.

### Preparation of (R)-4-(3-((1-(2-(4,4-dimethylpiperidin-1-yl)-3,6-dimethyl-4-oxo-4H-chromen-8-yl)ethyl)amino)pyridin-2-yl)-2-formylphenyl trifluoromethanesulfonate

A mixture of (R)-5-(3-((1-(2-(4,4-dimethylpiperidin-1-yl)-3,6-dimethyl-4-oxo-4H-chromen-8-yl)ethyl)amino)pyridin-2-yl)-2-hydroxybenzaldehyde (1.40 g, 2.66 mmol, 1 eq), PhNTf₂ (1.90 g, 5.33 mmol, 2 eq), TEA (539 mg, 5.33 mmol, 741 μL, 2 eq) and DMAP (32.5 mg, 266 μmol, 0.1 eq) in DCM (20 mL) was degassed and purged with N₂ for 3 times at 20° C., and then the mixture was stirred at 20° C. for 1 h under N₂ atmosphere. The reaction mixture was concentrated under reduced pressure to give a residue. The residue was purified by flash silica gel chromatography (Biotage®; 20 g SepaFlash® Silica Flash Column, Eluent of 30-45% Ethyl acetate/Petroleum ether gradient@80 mL/min) to give the title compound (1.30 g, 1.98 mmol, 53% yield by 2 steps) as a yellow solid. ¹H NMR (CDCl3, 400 MHz) δ 10.34 (s, 1H), 8.44 (d, J=2.4 Hz, 1H), 8.16-8.06 (m, 2H), 7.89 (d, J=0.8 Hz, 1H), 7.60-7.57 (m, 1H), 7.32 (d, J=2.0 Hz, 1H), 7.05-7.05 (m, 1H), 6.73-6.71 (m, 1H), 5.01-4.95 (m, 1H), 4.37-4.36 (m, 1H), 3.38 (t, J=4.8 Hz, 4H), 2.37 (s, 3H), 2.06 (s, 3H), 1.58 (d, J=6.8 Hz, 3H), 1.51 (t, J=5.6 Hz, 4H), 1.03 (s, 6H).

### Preparation of (R)-5-(3-((1-(2-(4,4-dimethylpiperidin-1-yl)-3,6-dimethyl-4-oxo-4H-chromen-8-yl)ethyl)amino)pyridin-2-yl)-2-(4,4,5,5-tetramethyl-1,3,2-dioxaborolan-2-yl)benzaldehyde

A mixture of (R)-4-(3-((1-(2-(4,4-dimethylpiperidin-1-yl)-3,6-dimethyl-4-oxo-4H-chromen-8-yl)ethyl)amino)pyridin-2-yl)-2-formylphenyl trifluoromethanesulfonate (600 mg, 912 μmol, 1 eq), B₂Pin₂ (1.16 g, 4.56 mmol, 5 eq), Pd(PPh₃)₂Cl₂ (64 mg, 91.2 μmol, 0.1 eq) and KOAc (179 mg, 1.82 mmol, 2 eq) in dioxane (10 mL) was degassed and purged with N₂ for 3 times at 20° C., and then the mixture was heated to 80° C. and stirred at 80° C. for 1 h under N₂ atmosphere. The reaction mixture was cooled to 25° C., filtered and the filtrate was concentrated under reduced pressure to give a residue. The residue was purified by flash silica gel chromatography (Biotage®; 20 g SepaFlash® Silica Flash Column, Eluent of 25~45% Ethyl acetate/Petroleum ether gradient@80 mL/min) to give the title compound (550 mg, containing some B₂Pin₂) as a yellow solid. ¹H NMR (CDCl₃, 400 MHz) δ 10.62 (s, 1H), 8.36 (d, J=1.6 Hz, 1H), 8.07-8.05 (m, 2H), 7.99-7.98 (m, 1H), 7.88 (s, 1H), 7.30 (d, J=2.0 Hz, 1H), 7.04-7.04 (m, 1H), 6.71-6.69 (m, 1H), 4.95-4.91 (m, 1H), 4.47-4.43 (m, 1H), 3.40-3.33 (m, 4H), 2.37 (s, 3H), 2.05 (s, 3H), 1.54-1.52 (m, 3H), 1.52-1.48 (m, 4H), 1.44 (s, 12H), 1.02 (s, 6H).

### Preparation of (R)-2-(4,4-dimethylpiperidin-1-yl)-8-(1-((2-(1-hydroxy-1H-benzo[d]oxazaborinin-6-yl)pyridin-3-yl)amino)ethyl)-3,6-dimethyl-4H-chromen-4-one

A mixture of (R)-5-(3-((1-(2-(4,4-dimethylpiperidin-1-yl)-3,6-dimethyl-4-oxo-4H-chromen-8-yl)ethyl)amino)pyridin-2-yl)-2-(4,4,5,5-tetramethyl-1,3,2-dioxaborolan-2-yl)benzaldehyde (550 mg, 865 mol, 1 eq), KOAc (169 mg, 1.73 mmol, 2 eq) and NH₂OH·HCl (120 mg, 1.73 mmol, 2 eq) in EtOH (5 mL)/H₂O (1 μL) was degassed and purged with N₂ for 3 times at 20° C., and then the mixture was stirred at 20° C. for 2 h under N₂ atmosphere. The reaction mixture was concentrated under reduced pressure to give a residue. The residue was purified by prep-HPLC (column: Phenomenex Gemini-NX 80×40 mm×3 um; mobile phase: [H2O (10 mM □H₄HCO₃)-AC□]; gradient:10%-50% B over 8.0 min) to give the title compound (266.7 mg, 476.03 μmol, 53.11% yield by 2 steps) as a white solid. ¹H NMR (DMSO-d₆, 400 MHz) δ 9.70-9.34 (m, 1H), 8.66 (s, 1H), 8.14-8.13 (m, 1H), 8.04-8.04 (m, 2H), 7.95-7.93 (m, 1H), 7.61 (s, 1H), 7.49 (s, 1H), 7.11-7.07 (m, 1H), 6.90-6.88 (m, 1H), 5.56-5.55 (m, 1H), 4.98-4.90 (m, 1H), 3.29-3.24 (m, 4H), 2.31 (s, 3H), 1.89 (s, 3H), 1.50 (d, J=6.8 Hz, 3H), 1.40-1.37 (m, 4H), 0.95 (s, 6H). MS (ESI): mass calcd. For C₃₂H₃₅BN₄O₄ 550.47, m/z found 551.2 [M+H]⁺. HPLC: 98.25% (220 nm), 99.55% (254 nm). Chiral purity: 96.72 ee %.

### Example 35

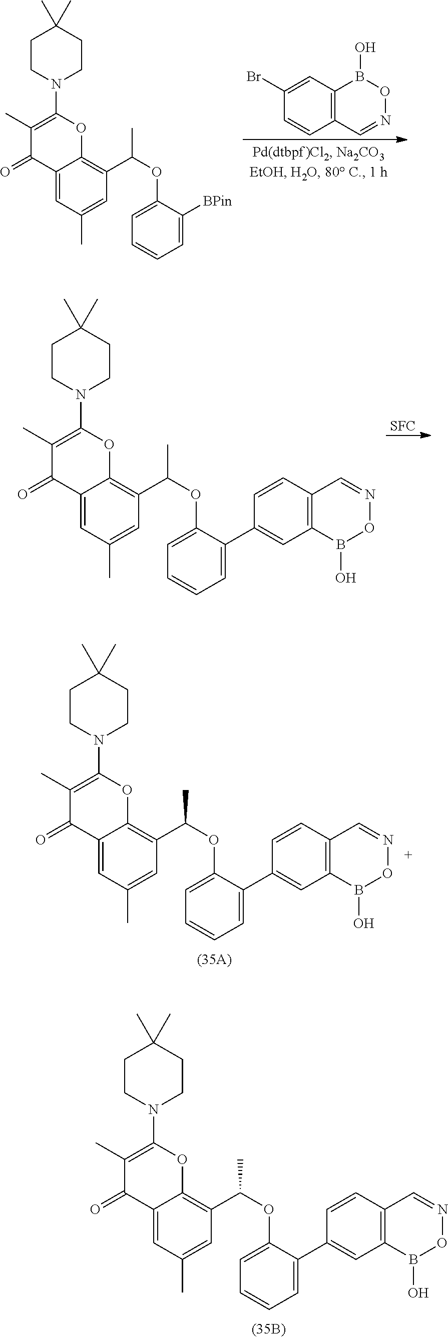

### Preparation of 2-(4,4-dimethyl-1-piperidyl)-8-[1-[2-(1-hydroxy-2,3,1-benzoxazaborinin-7-yl)phenoxy]ethyl]-3,6-dimethyl-chromen-4-one

A mixture of 2-(4,4-dimethyl-1-piperidyl)-3,6-dimethyl-8-[1-[2-(4,4,5,5-tetramethyl-1,3,2-dioxaborolan-2-yl)phenoxy]ethyl]chromen-4-one (100 mg, 188 μmol, 1 eq), 7-bromo-1-hydroxy-2,3,1-benzoxazaborinine (63.7 mg, 282 μmol, 1.5 eq), Na₂CO₃ (59.8 mg, 564 μmol, 3 eq) and Pd(dtbpf)Cl₂ (12.2 mg, 18.8 μmol, 0.1 eq) in EtOH (1 mL)/H₂O (0.2 mL) was degassed and purged with N₂ for 3 times, and then the reaction mixture was directly stirred at 80° C. (the oil-bath was pre-heated to 80° C.) for 1 h under N₂ atmosphere. The reaction mixture was cooled to 20° C. and filtered, the filtrate was directly purified by prep-HPLC (column: Waters Xbridge BEH C18 100×30 mm×10 um; mobile phase: [H₂O (10 mM NH₄HCO₃)-AC□]; gradient: 60%-90% B over 8.0 min) to give the title compound (7.6 mg, 13.8 μmol, 7.34% yield) as an off-white solid. ¹H NMR (DMSO-d₆, 400 MHz) δ 9.35 (s, 1H), 8.67 (s, 1H), 8.21 (s, 1H), 7.93 (dd, J=1.2, 8.0 Hz, 1H), 7.74 (d, J=8.0 Hz, 1H), 7.63 (s, 1H), 7.38 (d, J=7.6 Hz, 1H), 7.34 (d, J=1.2 Hz, 1H), 7.33-7.29 (m, 1H), 7.09 (s, 1H), 7.08-7.05 (m, 1H), 5.82 (q, J=6.4 Hz, 1H), 3.27-3.16 (m, 4H), 2.26 (s, 3H), 1.87 (s, 3H), 1.61 (d, J=6.0 Hz, 3H), 1.35-1.34 (m, 4H), 0.93 (s, 6H). MS (ESI): mass calcd. For C₃₃H₃₅BN₂O₅ 550.26, m/z found 551.3 [M+H]⁺. HPLC: 98.43% (220 nm), 98.10% (254 nm).

### Preparation of 2-(4,4-dimethyl-1-piperidyl)-8-[(1S)-1-[2-(1-hydroxy-2,3,1-benzoxazaborinin-7-yl)phenoxy]ethyl]-3,6-dimethyl-chromen-4-one and 2-(4,4-dimethyl-1-piperidyl)-8-[(1R)-1-[2-(1-hydroxy-2,3,1-benzoxazaborinin-7-yl)phenoxy]ethyl]-3,6-dimethyl-chromen-4-one

2-(4,4-dimethyl-1-piperidyl)-8-[1-[2-(1-hydroxy-2,3,1-benzoxazaborinin-7-yl)phenoxy]ethyl]-3,6-dimethyl-chromen-4-one (60 mg) was separated by SFC (column: ChiralPak IH, 250×30 mm×10 um; mobile phase: [CO2-MeOH]; B %:30%, isocratic elution mode) to give the title compound, Isomer 1 (15 mg, 27.2 μmol, 25% yield) as a white solid and the title compound, Isomer 2 (17.3 mg, 31.4 μmol, 28.8% yield) as a white solid. Isomer 1: ¹H NMR (DMSO-d₆, 400 MHz) δ 9.36 (s, 1H), 8.67 (s, 1H), 8.21 (s, 1H), 7.93 (dd, J=1.6, 8.0 Hz, 1H), 7.74 (d, J=8.0 Hz, 1H), 7.63 (d, J=1.6 Hz, 1H), 7.38 (d, J=7.6 Hz, 1H), 7.34 (d, J=1.6 Hz, 1H), 7.33-7.32 (m, 1H), 7.09 (s, 1H), 7.08-7.07 (m, 1H), 5.82 (q, J=6.0 Hz, 1H), 3.29-3.22 (m, 4H), 2.26 (s, 3H), 1.87 (s, 3H), 1.61 (d, J=6.4 Hz, 3H), 1.38-1.29 (m, 4H), 0.93 (s, 6H). MS (ESI): mass calcd. For C₃₃H₃₅BN₂O₅ 550.26, m/z found 551.3 [M+H]⁺. HPLC: 98.13% (220 nm), 98.21% (254 nm). Chiral purity: 99.50% ee. Isomer 2: ¹H NMR (DMSO-d₆, 400 MHz) δ 9.36 (s, 1H), 8.67 (s, 1H), 8.21 (s, 1H), 7.94 (dd, J=1.2, 8.0 Hz, 1H), 7.74 (d, J=8.0 Hz, 1H), 7.63 (s, 1H), 7.39 (d, J=6.8 Hz, 1H), 7.34 (s, 1H), 7.32-7.30 (m, 1H), 7.10 (s, 1H), 7.08-7.07 (m, 1H), 5.82 (q, J=6.4 Hz, 1H), 3.27-3.19 (m, 4H), 2.26 (s, 3H), 1.87 (s, 3H), 1.61 (d, J=6.4 Hz, 3H), 1.39-1.34 (m, 4H), 0.94 (s, 6H). MS (ESI): mass calcd. For C₃₃H₃₅BN₂O₅ 550.26, m/z found 551.3 [M+H]⁺. HPLC: 97.89% (220 nm), 97.41% (254 nm). Chiral purity: 97.72% ee.

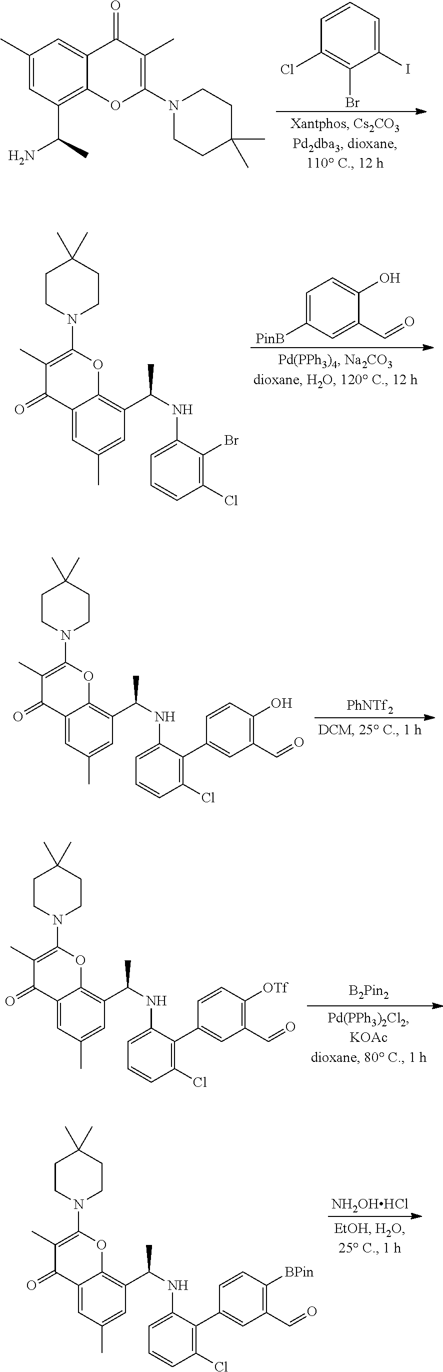

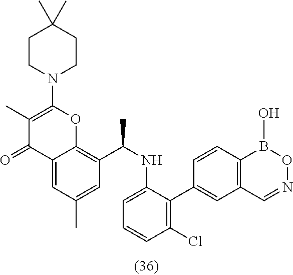 [SMILES: Cc1cc([C@@H](C)Nc2cccc(Cl)c2-c2ccc3c(c2)C=NOB3O)c2oc(N3CCC(C)(C)CC3)c(C)c(=O)c2c1]

### Example 36

### Preparation of 8-[(1R)-1-(2-bromo-3-chloro-anilino)ethyl]-2-(4,4-dimethyl-1-piperidyl)-3,6-dimethy l-chromen-4-one

The title compound was prepared as yellow oil (1.00 g, 1.93 mmol, 90.60% yield) according to the similar procedure in Example 78, using 8-[(1R)-1-aminoethyl]-2-(4,4-dimethyl-1-piperidyl)-3,6-dimethyl-chromen-4-one (700 mg, 2.13 mmol, 1 eq) and 2-bromo-1-chloro-3-iodo-benzene (1.35 g, 4.26 mmol, 2 eq). ¹H NMR (DMSO-d₆, 400 MHz) δ 7.63 (s, 1H), 7.45 (s, 1H), 7.06 (t, J=8.0 Hz, 1H), 6.79 (d, J=7.6 Hz, 1H), 6.39 (d, J=8.4 Hz, 1H), 5.58 (d, J=6.8 Hz, 1H), 5.05 (t, J=6.4 Hz, 1H), 3.39-3.35 (m, 4H), 2.31 (s, 3H), 1.63-1.62 (m, 4H), 1.90 (s, 3H), 1.62 (d, J=6.4 Hz, 3H), 0.96 (s, 6H).

### Preparation of 5-[2-chloro-6-[[(1R)-1-[2-(4,4-dimethyl-1-piperidyl)-3,6-dimethyl-4-oxo-chromen-8-yl]ethyl]amino]phenyl]-2-hydroxy-benzaldehyde

The title compound was prepared as a white solid (190 mg, 339.84 μmol, 35.20% yield) according to the procedures described herein and the synthetic scheme, using 8-[(1R)-1-(2-bromo-3-chloro-anilino)ethyl]-2-(4,4-dimethyl-1-piperidyl)-3,6-dimethyl-chrome n-4-one (500 mg, 965.47 μmol, 1 eq) and 2-hydroxy-5-(4,4,5,5-tetramethyl-1,3,2-dioxaborolan-2-yl)benzaldehyde (479 mg, 1.93 mmol, 2 eq). ¹H NMR (DMSO-d₆, 400 MHz) δ 10.93 (s, 1H), 10.29 (s, 1H), 7.60 (s, 1H), 7.52 (d, J=2.0 Hz, 1H), 7.50-7.40 (m, 1H), 7.31 (s, 1H), 7.14 (d, J=8.4 Hz, 1H), 7.05 (d, J=2.8 Hz, 1H) 6.74 (d, J=8.0 Hz, 1H), 6.47-6.44 (m, 1H), 4.99-4.91 (m, 1H), 4.52 (d, J=6.8 Hz, 1H), 3.39-3.35 (m, 4H), 2.31 (s, 3H), 1.90 (s, 3H), 1.44-1.39 (m, 7H), 0.97 (s, 6H).

### Preparation of [4-[2-chloro-6-[[(1R)-1-[2-(4,4-dimethyl-1-piperidyl)-3,6-dimethyl-4-oxo-chromen-8-yl]ethyl]amino]phenyl]-2-formyl-phenyl]trifluoromethanesulfonate

The title compound was prepared as yellow oil (120 mg, 173.62 μmol, 69.34% yield) according to the procedures described herein and the synthetic scheme, using 5-[2-chloro-6-[[(1R)-1-[2-(4,4-dimethyl-1-piperidyl)-3,6-dimethyl-4-oxo-chromen-8-yl]ethyl]amino]phenyl]-2-hydroxy-benzaldehyde (140 mg, 250 gmol) and PhNTf₂ (98 mg, 275.45 μmol, 1.1 eq). ¹H NMR (DMSO-d₆, 400 MHz) δ 10.15-10.12 (m, 1H), 8.06 (s, 1H), 7.86-7.81 (m, 1H), 7.76 (t, J=8.0 Hz, 1H) 7.61 (s, 1H), 7.41 (t, J=7.6 Hz, 1H), 7.35 (dd, J=2.0 Hz, 10.4 Hz, 1H), 7.28 (t, J=8.8 Hz, 1H), 7.12 (t, J=8.0 Hz, 1H), 5.03-4.86 (m, 2H), 3.39-3.36 (m, 4H), 2.31 (d, J=4.0 Hz, 3H), 1.91 (s, 3H), 1.45 (t, J=5.2 Hz, 4H), 1.40 (d, J=6.0 Hz, 3H), 0.98 (s, 6H).

### Preparation of 5-[2-chloro-6-[[(1R)-1-[2-(4,4-dimethyl-1-piperidyl)-3,6-dimethyl-4-oxo-chromen-8-yl]ethyl]amino]phenyl]-2-(4,4,5,5-tetramethyl-1,3,2-dioxaborolan-2-yl)benzaldehyde

The title compound was prepared as a white solid (500 mg, crude) according to the procedures described herein and the synthetic scheme, using [4-[2-chloro-6-[[(1R)-1-[2-(4,4-dimethyl-1-piperidyl)-3,6-dimethyl-4-oxo-chromen-8-yl]ethyl]amino]phenyl]-2-formyl-phenyl]trifluoromethanesulfonate (600 mg, 87 gmol) and B₂Pin₂ (110 mg, 434.06 μmol, 5 eq). The product was used directly for next step without further purification.

### Preparation of 8-[(1R)-1-[3-chloro-2-(1-hydroxy-2,3,1-benzoxazaborinin-6-yl) aniline]ethyl]-2-(4,4-dimethyl-1-piperidyl)-3,6-dimethyl-chromen-4-one-

The title compound was prepared as a white solid (9.80 mg, 16.78 μmol, 22.46% yield) according to the procedures described herein and the synthetic scheme, using 5-[2-chloro-6-[[(1R)-1-[2-(4,4-dimethyl-1-piperidyl)-3,6-dimethyl-4-oxo-chromen-8-yl]ethyl]amino]phenyl]-2-(4,4,5,5-tetramethyl-1,3,2-dioxaborolan-2-yl)benzaldehyde (50 mg, 75 μmol, 1 eq) and NH₂OH·HCl (16 mg, 224.20 μmol, 3 eq). ¹H NMR (DMSO-d₆, 400 MHz) δ 8.68 (d, J=2.4 Hz, 1H), 8.20 (dd, J=5.2 Hz, 8.0 Hz, 1H), 7.67-7.57 (m, 3H), 7.33 (dd, J=2.0 Hz, 6.0 Hz, 1H), 7.13-7.08 (m, 1H), 6.79 (dd, J=2.0 Hz, 8.0 Hz, 1H), 6.52 (dd, J=8.4 Hz, 12.8 Hz, 1H), 4.94 (s, 1H), 4.56 (s, 1H), 3.36-3.23 (m, 4H), 2.32 (d, J=2.0 Hz, 3H), 1.90 (d, J=3.2 Hz, 3H), 1.40-1.37 (m, 7H), 0.96 (s, 6H). MS (ESI): mass calcd. For C₃₃H₃₅BClN₃O₄ 583.24, m/z found 584.3 [M+H]⁺. HPLC: 96.26% (220 nm), 97.91% (254 nm). Chiral purity: 100% ee.

### Example 37

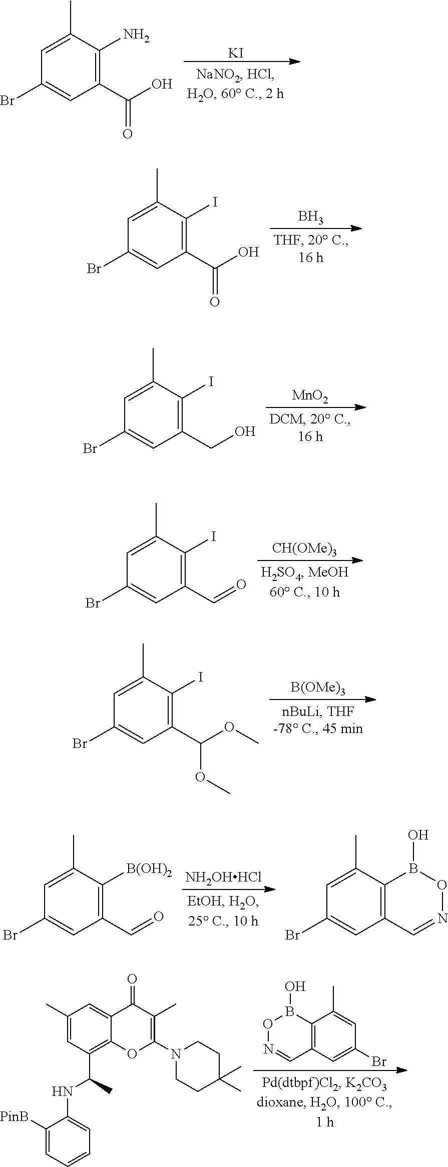

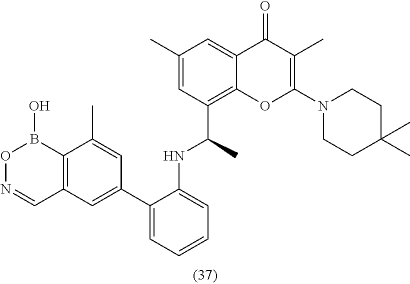 [SMILES: Cc1cc([C@@H](C)Nc2ccccc2-c2cc(C)c3c(c2)C=NOB3O)c2oc(N3CCC(C)(C)CC3)c(C)c(=O)c2c1]

### Preparation of 5-bromo-2-iodo-3-methylbenzoic acid

To a solution of 2-amino-5-bromo-3-methylbenzoic acid (2.00 g, 8.69 mmol, 1 eq) in HCl (3 M, 20.0 mL) was slowly added NaNO₂ (779 mg, 11.3 mmol, 1.3 eq) in portions at 0° C., the reaction mixture was stirred at 0° C. for 0.5 h. Then a solution of KI (1.73 g, 10.4 mmol, 1.2 eq) in H₂O (4 mL) was added dropwise to the reaction mixture above at 0° C. The resulting reaction mixture was heated to 60° C. and stirred at 60° C. for 1.5 h. The reaction mixture was cooled to 25° C., filtered and the filter cake was dried in vacuo to give the title compound (2.00 g, crude) as a yellow solid, which was used directly for next step without further purification. ¹H NMR (DMSO-d₆ 400 MHz) δ 7.67 (d, J=2.0 Hz, 1H), 7.49 (d, J=2.0 Hz, 1H), 2.43 (s, 3H).

### Preparation of (5-bromo-2-iodo-3-methylphenyl)methanol

To a solution of 5-bromo-2-iodo-3-methylbenzoic acid (2.00 g, 5.87 mmol, 1 eq) in THF (20 mL) was added BH₃·THF (1 M, 29.3 mL, 5 eq) dropwise at 0° C., the reaction mixture was stirred at 20° C. for 16 h. The reaction mixture was quenched by H₂O (30 mL) at 0° C., and then extracted with EtOAc (30 mL×3). The combined organic layers were washed with brine (30 mL), dried over anhydrous Na₂SO₄, filtered and concentrated under reduced pressure to give a residue. The residue was purified by flash silica gel chromatography (Biotage®; 20 g SepaFlash® Silica Flash Column, Eluent of 5~15% Ethyl acetate/Petroleum ether gradient@50 mL/min) to give the title compound (1.10 g, 3.36 mmol, 57.35% yield) as a white solid. ¹H NMR (CDCl₃ 400 MHz) δ 7.45 (s, 1H), 7.34 (d, J=2.0 Hz, 1H), 4.68 (s, 2H), 2.45 (s, 3H).

### Preparation of 5-bromo-2-iodo-3-methylbenzaldehyde

To a solution of (5-bromo-2-iodo-3-methylphenyl)methanol (1.10 g, 3.36 mmol, 1 eq) in DCM (10 mL) was added MnO₂ (2.05 g, 23.5 mmol, 7 eq) in one portion at 20° C., the resulting reaction mixture was stirred at 20° C. for 16 h. The reaction mixture was filtered and the filter cake was washed with DCM (10 mL×2). The filtrate was concentrated under reduced pressure to give the title compound (850 mg, 2.62 mmol, 77.75% yield) as a white solid. ¹H NMR (CDCl₃ 400 MHz) δ 10.15 (s, 1H), 7.79 (d, J=2.0 Hz, 1H), 7.62 (d, J=2.4 Hz, 1H), 2.52 (s, 3H).

### Preparation of 5-bromo-1-(dimethoxymethyl)-2-iodo-3-methyl-benzene

To a solution of 5-bromo-2-iodo-3-methyl-benzaldehyde (0.7 g, 2.15 mmol, 1 eq) and trime thoxymethane (457 mg, 4.31 mmol, 472.33 μL, 2 eq) in MeOH (10 mL) was added H₂SO₄ (22 mg, 215.42 μmol, 11.72 μL, 0.1 eq) in one portion at 25° C., the reaction mixture was heated to 60° C. and stirred at 60° C. for 10 h. The reaction mixture was cooled to 0° C., quenched with sat.aq.NaHCO₃ (10 mL) and extracted with EtOAc (15 mL×3). The combined organic layers were washed with brine (10 mL×2), dried over Na₂SO₄, filtered and concentrated under reduced pressure to give the title compound (0.85 g, crude) as colorless oil. ¹H NMR (CDCl₃ 400 MHz) δ7.51 (s, 1H), 7.39 (s, 1H), 5.43 (s, 1H), 3.39 (s, 6H), 2.47 (s, 3H).

### Preparation of (4-bromo-2-formyl-6-methyl-phenyl)boronic acid

To a solution of 5-bromo-1-(dimethoxymethyl)-2-iodo-3-methyl-benzene (0.75 g, 2.02 mmol, 1 eq) in THF (10 mL) was added n-BuLi (2.5 M, 808.61 μL, 1 eq) dropwise at −78° C., the reaction mixture was stirred at −78° C. for 15 min. Then trimethyl borate (210 mg, 2.02 mmol, 228.33 μL, 1 eq) was added to the reaction mixture dropwise at −78° C. and the reaction mixture was stirred at −78° C. for 30 min. The reaction mixture was quenched with HCl (10 mL, 3 M) and extracted with EtOAc (15 mL×3). The combined organic phase was washed with brine (10 mL), dried over Na₂SO₄, filtered and concentrated in vacuum to give the title compound (0.49 g, crude) as light yellow oil, which was used directly without further purification.

### Preparation of 6-bromo-1-hydroxy-8-methyl-2,3,1-benzoxazaborinine

To a solution of (4-bromo-2-formyl-6-methyl-phenyl)boronic acid (0.49 g, 2.02 mmol, 1 eq) in EtOH (5 mL)/H₂O (1 mL) was added NH₂OH·HCl (281 mg, 4.04 mmol, 2 eq) in one portion at 25° C., the reaction mixture was stirred at 25° C. for 10 h. The reaction suspension was filtered and the filter cake was dried in vacuo to give the title compound (0.26 g, 1.08 mmol, 53.73% yield) as a white solid. ¹H NMR (CDCl₃ 400 MHz) δ 9.37 (s, 1H), 8.57 (s, 1H), 7.82 (s, 1H), 7.74 (s, 1H), 2.63 (s, 3H).

### Preparation of 2-(4,4-dimethyl-1-piperidyl)-8-[(1R)-1-[2-(1-hydroxy-8-methyl-2,3,1-benzoxazaborinin-6-yl)anilino]ethyl]-3,6-dimethyl-chromen-4-one

The title compound was prepared as a white solid (16.1 mg, 10.61% yield) according to the procedure described in preceding examples, using 6-bromo-1-hydroxy-8-methyl-2,3,1-benzoxazaborinine (64.5 mg, 269 μmol, 1 eq) and 2-(4,4-dimethyl-1-piperidyl)-3,6-dimethyl-8-[(1R)-1-[2-(4,4,5,5-tetramethyl-1,3,2-dioxaborolan-2-yl)anilino]ethyl]chromen-4-one (200 mg, 377 μmol, 1.4 eq). ¹H NMR (DMSO-d₆, 400 MHz) δ 9.22 (br s, 1H), 8.57 (s, 1H), 7.61 (s, 2H), 7.54 (s, 1H), 7.47 (s, 1H), 7.09 (t, J=8.0 Hz, 1H), 7.03 (d, J=6.4 Hz, 1H), 6.70 (t, J=7.2 Hz, 1H), 6.59 (d, J=8.0 Hz, 1H), 5.03 (d, J=7.2 Hz, 1H), 4.95-4.90 (m, 1H), 3.27-3.18 (m, 4H), 2.63 (s, 3H), 2.32 (s, 3H), 1.89 (s, 3H), 1.47 (d, J=6.8 Hz, 3H), 1.37 (t, J=5.2 Hz, 4H), 0.94 (s, 6H). MS (ESI): mass calcd. For C₃₄H₃₈BN₃O₄ 563.30, m/z found 564.2 [M+H]⁺. HPLC: 98.23% (220 nm), 98.19% (254 nm).

### Example 38

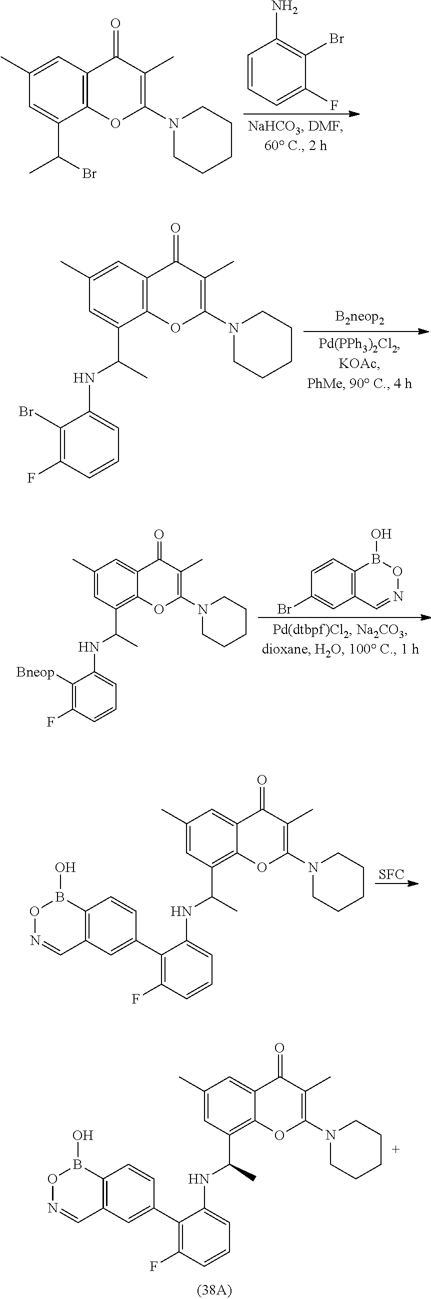

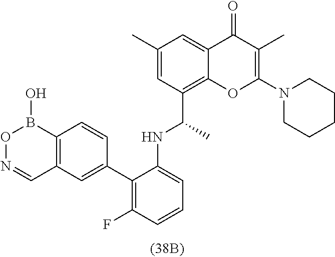 [SMILES: Cc1cc([C@H](C)Nc2cccc(F)c2-c2ccc3c(c2)C=NOB3O)c2oc(N3CCCCC3)c(C)c(=O)c2c1]

### Preparation of 8-(1-((2-bromo-3-fluorophenyl)amino)ethyl)-3,6-dimethyl-2-(piperidin-1-yl)-4H-chromen-4-one

A mixture of 8-(1-bromoethyl)-3,6-dimethyl-2-(piperidin-1-yl)-4H-chromen-4-one (1.00 g, 2.75 mmol, 1 eq), 2-bromo-3-fluoroaniline (1.04 g, 5.49 mmol, 2 eq) and NaHCO₃ (461 mg, 5.49 mmol, 213 μL, 2 eq) in DMF (10 mL) was degassed and purged with N₂ for 3 times at 20° C., and then the reaction mixture was stirred at 60° C. for 2 h under N₂ atmosphere. The reaction mixture was quenched by H₂O (30 mL) at 20° C. and then filtered. The filter cake was triturated with MTBE (10 mL) and dried in vacuo to give the title compound (800 mg, 1.69 mmol, 61.56% yield) as a yellow solid. ¹H NMR (CDCl₃, 400 MHz) δ 7.87 (s, 1H), 7.33 (d, J=2.0 Hz, 1H), 6.98-6.95 (m, 1H), 6.48-6.44 (m, 1H), 6.09 (d, J=8.4 Hz, 1H), 5.03-4.97 (m, 1H), 4.85-4.83 (m, 1H), 3.39-3.38 (m, 4H), 2.36 (s, 3H), 2.06 (s, 3H), 1.73-1.71 (m, 6H), 1.68-1.66 (m, 3H).

### Preparation of 8-(1-((2-(5,5-dimethyl-1,3,2-dioxaborinan-2-yl)-3-fluorophenyl)amino) ethyl)-3,6-dimethyl-2-(piperidin-1-yl)-4H-chromen-4-one

A mixture of 8-(1-((2-bromo-3-fluorophenyl)amino)ethyl)-3,6-dimethyl-2-(piperidin-1-yl)-4H-chromen-4-one (400 mg, 844 μmol, 1 eq), B₂(neop)₂ (954 mg, 4.22 mmol, 5 eq), KOAc (165 mg, 1.69 mmol, 2 eq) and Pd(PPh₃)₂Cl₂ (59 mg, 84.5 μmol, 0.1 eq) in PhMe (4 mL) was degassed and purged with N₂ for 3 times at 20° C., and then the reaction mixture was stirred at 90° C. for 4 h under N₂ atmosphere. The reaction mixture was filtered and the filtrate ws concentrated under reduced pressure to give a residue. The residue was purified by flash silica gel chromatography (BIOTAGE®; 4 g SepaFlash@Silica Flash Column, Eluent of 30~50% Ethyl acetate/Petroleum ether gradient@36 mL/min) to give the title compound (550 mg, crude) as a red solid.

### Preparation of 8-(1-((3-fluoro-2-(1-hydroxy-1H-benzo[d][1,2,6]oxazaborinin-6-yl) phenyl)amino)ethyl)-3,6-dimethyl-2-(piperidin-1-yl)-4H-chromen-4-one

A mixture of 8-(1-((2-(5,5-dimethyl-1,3,2-dioxaborinan-2-yl)-3-fluorophenyl)amino)ethyl)-3,6-dimethyl-2-(piperidin-1-yl)-4H-chromen-4-one (400 mg, 912 μmol, 1 eq), 6-bromo-1H-benzo[d][1,2,6]oxazaborinin-1-ol (226 mg, 1.00 mmol, 1.1 eq), ditert-butyl(cyclopentyl)phosphane;dichloropalladium;iron (59 mg, 91 μmol, 0.1 eq) and Na₂CO₃ (193 mg, 1.83 mmol, 2 eq) in dioxane (1 mL) and H₂O (0.2 mL) was degassed and purged with N₂ for 3 times at 20° C., and then the mixture was stirred at 100° C. for 1 h under N₂ atmosphere. The reaction mixture was directly purified by prep-HPLC (column: Welch Xtimate C₁₈ 180×70 mm×10 um; mobile phase: [H₂O (0.2% FA)-AC□]; gradient: 30%-60% B over 20.0 min) to give the title compound (80 mg, 136.45 mol, 14.95% yield) as a white solid ¹H NMR (DMSO-d₆, 400 MHz) δ 8.55-8.51 (m, 1H), 8.15-8.08 (m, 1H), 7.64-7.55 (m, 2H), 7.46-7.37 (m, 2H), 7.10-7.06 (m, 1H), 6.52-6.33 (m, 2H), 4.97-4.94 (m, 1H), 4.70-4.46 (m, 1H), 3.32-3.32 (m, 4H), 2.32 (s, 3H), 1.90 (s, 3H), 1.58-1.58 (m, 6H), 1.41 (d, J=6.8 Hz, 3H).

### Preparation of (S)-8-(1-((3-fluoro-2-(1-hydroxy-1H-benzo[d][1,2,6]oxazaborinin-6-yl)phenyl)amino)ethyl)-3,6-dimethyl-2-(piperidin-1-yl)-4H-chromen-4-one and (R)-8-(1-((3-fluoro-2-(1-hydroxy-1H-benzo[d][1,2,6]oxazaborinin-6-yl)phenyl) amino) ethyl)-3,6-dimethyl-2-(piperidin-1-yl)-4H-chromen-4-one

8-(1-((3-fluoro-2-(1-hydroxy-1H-benzo[d][1,2,6]oxazaborinin-6-yl)phenyl)amino)ethyl)-3,6-dimethyl-2-(piperidin-1-yl)-4H-chromen-4-one (80 mg, 148 μmol, 1 eq) was purified by SFC (column: DAICEL CHIRALPAK IG (250 mm×30 mm, 10 um); mobile phase: [CO₂—IPA (0.1% NH₃H₂O)]; B %:50%, isocratic elution mode) to give the title compound, Isomer 1 (18.6 mg, 33.63 μmol, 22.67% yield) as a white solid and the title compound, Isomer 2 (33.3 mg, 61.30 μmol, 41.33% yield) as a white solid. Isomer 1: ¹H NMR (DMSO-d₆, 400 MHz) δ 9.49-9.39 (m, 1H), 8.67 (s, 1H), 8.18 (d, J=7.6 Hz, 1H), 7.74-7.71 (m, 2H), 7.60 (s, 1H), 7.40 (s, 1H), 7.14-7.09 (m, 1H), 6.52 (t, J=8.4 Hz, 1H), 6.38 (d, J=8.0 Hz, 1H), 4.98-4.91 (m, 2H), 3.26-3.22 (m, 4H), 2.32 (s, 3H), 1.89 (s, 3H), 1.58-1.58 (m, 6H), 1.42 (d, J=6.0 Hz, 3H). MS (ESI): mass calcd. For C₃₁H₃₁BFN₃O₄ 539.41, m/z found 540.2 [M+H]⁺. HPLC: 97.52% (220 nm), 99.19% (254 nm). Chiral purity: 100 ee %. Isomer 2: ¹H NMR (DMSO-d₆, 400 MHz) δ 8.18-7.94 (m, 2H), 7.61-7.59 (m, 2H), 7.37-7.05 (m, 3H), 6.52-6.46 (m, 1H), 6.32-6.28 (m, 1H), 4.96-4.50 (m, 2H), 3.32-3.32 (m, 4H), 2.32 (s, 3H), 1.90 (s, 3H), 1.58-1.58 (m, 6H), 1.42-1.40 (m, 3H). MS (ESI): mass calcd. For C₃₁H₃₁BFN₃O₄ 539.41, m/z found 540.2 [M+H]⁺. HPLC: 99.29% (220 nm), 99.57% (254 nm). Chiral purity: 98.06 ee %.

### Example 39

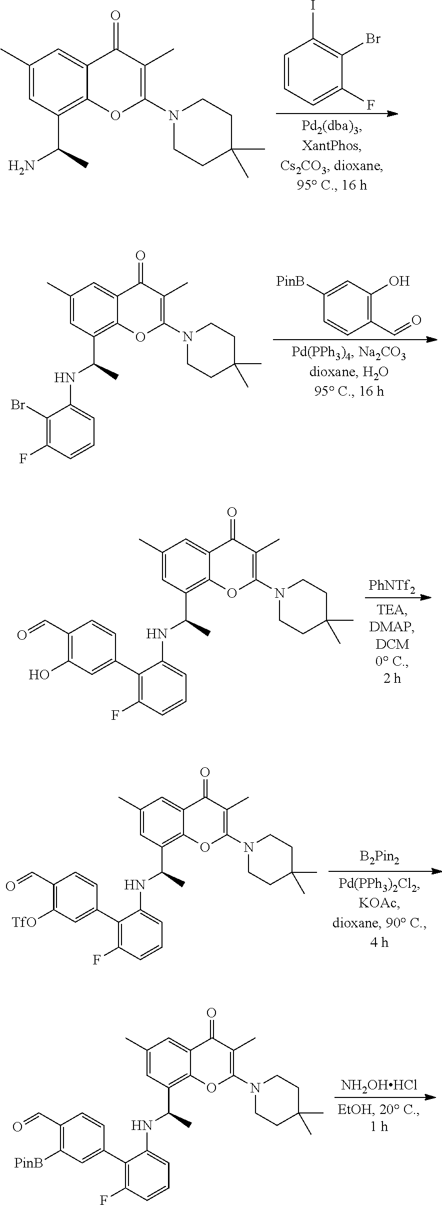

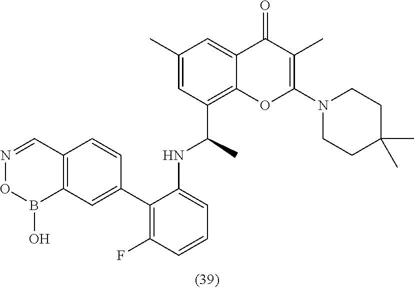 [SMILES: Cc1cc([C@@H](C)Nc2cccc(F)c2-c2ccc3c(c2)B(O)ON=C3)c2oc(N3CCC(C)(C)CC3)c(C)c(=O)c2c1]

### Preparation of 8-[(1R)-1-(2-bromo-3-fluoro-anilino)ethyl]-2-(4,4-dimethyl-1-piperidyl)-3,6-dimethyl-chromen-4-one

The title compound was prepared as a yellow solid (2.2 g, crude) according to the similar procedure of in Example 78, using 8-[(1R)-1-aminoethyl]-2-(4,4-dimethyl-1-piperidyl)-3,6-dimethyl-chromen-4-one (1.00 g, 3.04 mmol) and 2-bromo-1-fluoro-3-iodo-benzene (1.83 g, 6.09 mmol). ¹H NMR (CDCl₃, 400 MHz) δ 7.88 (d, J=1.2 Hz, 1H), 7.33 (d, J=2.0 Hz, 1H), 7.00-6.94 (m, 1H), 6.48-6.44 (m, 1H), 6.11 (d, J=4.0 Hz, 1H), 5.03-4.99 (m, 1H), 4.85-4.84 (m, 1H), 3.41-3.38 (m, 4H), 2.37 (s, 3H), 2.06 (s, 3H), 1.68 (d, J=6.4 Hz, 3H), 1.53-1.52 (m, 4H), 1.04 (s, 6H).

### Preparation of 4-[2-[[(1R)-1-[2-(4,4-dimethyl-1-piperidyl)-3,6-dimethyl-4-oxo-chromen-8-yl]ethyl]amino]-6-fluoro-phenyl]-2-hydroxy-benzaldehyde

The title compound was prepared as a yellow solid (600 mg, 1.11 mmol, 55.44% yield) according to the procedures described herein and the synthetic scheme, using 8-[(1R)-1-(2-bromo-3-fluoro-anilino)ethyl]-2-(4,4-dimethyl-1-piperidyl)-3,6-dimethyl-chromen-4-one (1.00 g, 1.99 mmol) and 2-hydroxy-4-(4,4,5,5-tetramethyl-1,3,2-dioxaborolan-2-yl)benzaldehyde (1.48 g, 5.98 mmol). ¹H NMR (CDCl₃, 400 MHz) δ 11.15 (s, 1H), 9.98 (s, 1H), 7.89 (s, 1H), 7.72 (d, J=8.0 Hz, 1H), 7.11-7.02 (m, 4H), 6.50 (t, J=8.4 Hz, 1H), 6.18 (d, J=8.0 Hz, 1H), 4.99-4.95 (m, 1H), 3.37-3.34 (m, 4H), 2.37 (s, 3H), 2.05 (s, 3H), 1.51-1.49 (m, 4H), 1.27 (s, 3H), 1.03 (s, 6H).

### Preparation of [5-[2-[[(1R)-1-[2-(4,4-dimethyl-1-piperidyl)-3,6-dimethyl-4-oxo-chromen-8-yl]ethyl]amino]-6-fluoro-phenyl]-2-formyl-phenyl]trifluoromethanesulfonate

The title compound was prepared as a yellow solid (1.20 g, 1.78 mmol, 96.51% yield) according to the procedures described herein and the synthetic scheme, using 4-[2-[[(1R)-1-[2-(4,4-dimethyl-1-piperidyl)-3,6-dimethyl-4-oxo-chromen-8-yl]ethyl]amino]-6-fluoro-phenyl]-2-hydroxy-benzaldehyde (1.00 g, 1.84 mmol, 1 eq). ¹H NMR (CDCl₃, 400 MHz) δ10.35 (s, 1H), 8.18 (d, J=8.0 Hz, 1H), 7.87 (s, 1H), 7.66 (d, J=8.0 Hz, 1H), 7.60 (s, 1H), 7.27-7.26 (m, 1H), 7.12-7.06 (m, 1H), 6.52 (t, J=8.8 Hz, 1H), 6.20 (d, J=8.4 Hz, 1H), 5.01-4.96 (m, 1H), 4.07 (d, J=5.2 Hz, 1H), 3.39-3.36 (m, 4H), 2.36 (s, 3H), 2.06 (s, 3H), 1.52-1.47 (m, 7H), 1.03 (s, 6H).

### Preparation of 4-[2-[[(1R)-1-[2-(4,4-dimethyl-1-piperidyl)-3,6-dimethyl-4-oxo-chromen-8-yl]ethyl]amino]-6-fluoro-phenyl]-2-(4,4,5,5-tetranethyl-1,3,2-dioxaborolan-2-yl)benzaldehyde

The title compound was prepared as a yellow solid (1.2 g, crude) according to the procedures described herein and the synthetic scheme, using [5-[2-[[(1R)-1-[2-(4,4-dimethyl-1-piperidyl)-3,6-dimethyl-4-oxo-chromen-8-yl]ethyl]amino]-6-fluoro-phenyl]-2-formyl-phenyl]trifluoromethanesulfonate (1.00 g, 1.48 mmol, 1 eq) and B₂Pin₂. The product was used directly for next step without further purification.

### Preparation of 2-(4,4-dimethyl-1-piperidyl)-8-[(1R)-1-[3-fluoro-2-(1-hydroxy-2,3,1-benzoxazaborinin-7-yl)anilino]ethyl]-3,6-dimethyl-chromen-4-one

The title compound was prepared as a white solid (15.9 mg, 27.58 μmol, 3.60% yield) according to the procedures described herein and the synthetic scheme, using 4-[2-[[(1R)-1-[2-(4,4-dimethyl-1-piperidyl)-3,6-dimethyl-4-oxo-chromen-8-yl]ethyl]amino]-6-fluoro-phenyl]-2-(4,4,5,5-tetramethyl-1,3,2-dioxaborolan-2-yl)benzaldehyde (500 mg, 766 μmol, 1 eq). ¹H NMR (DMSO-d₆, 400 MHz) δ 9.42 (s, 1H), 8.71 (s, 1H), 8.05 (s, 1H), 7.86 (d, J=8.0 Hz, 1H), 7.79 (d, J=8.0 Hz, 1H), 7.60 (s, 1H), 7.39 (d, J=1.6 Hz, 1H), 7.12-7.08 (m, 1H), 6.52 (d, J=8.8 Hz, 1H), 6.36 (d, J=8.8 Hz, 1H), 4.96 (s, 2H), 3.31-3.30 (m, 4H), 2.32 (s, 3H), 1.90 (s, 3H), 1.42-1.40 (m, 7H), 0.96 (s, 6H). MS (ESI): mass calcd. For C₃₃H₃₅BFN₃O₄ 567.27, m/z found 568.3 [M+H]⁺. HPLC: 98.44% (220 nm), 97.31% (254 nm). Chiral purity: 98.38% ee.

### Example 40

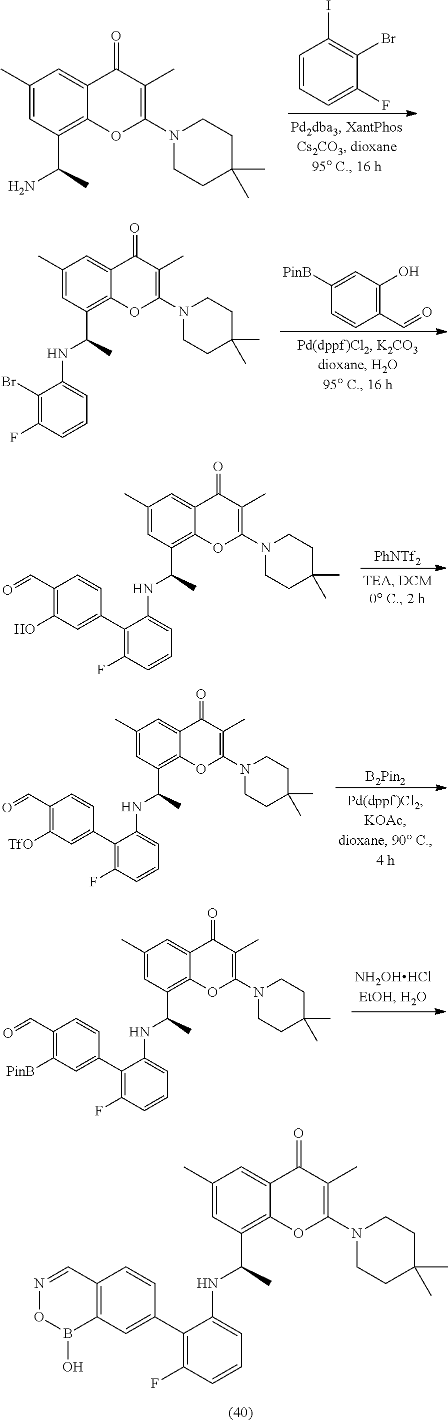

### Preparation of 8-[(1R)-1-(2-bromo-3-fluoro-anilino)ethyl]-2-(4,4-dimethyl-1-piperidyl)-3,6-dimethyl-chromen-4-one

The title compound was prepared as yellow solid (2.2 g, crude)) according to the similar procedure of in Example 78, using 8-[(1R)-1-aminoethyl]-2-(4,4-dimethyl-1-piperidyl)-3,6-dimethyl-chromen-4-one (1.00 g, 3.04 mmol, 1 eq) and 2-bromo-1-fluoro-3-iodo-benzene (1.83 g, 6.09 mmol, 2 eq). ¹H NMR (CDCl₃, 400 MHz) δ 7.88 (s, 3H), 7.34 (s, 3H), 6.98-6.94 (m, 1H), 6.48-6.44 (m, 1H), 6.12-6.10 (m, 1H), 5.02-4.97 (m, 1H), 4.85-4.84 (m, 1H), 3.41-3.83 (m, 4H), 2.37 (s, 3H), 2.06 (s, 3H), 1.68 (d, J=6.4 Hz, 3H), 1.53-1.50 (m, 4H), 1.03 (s, 6H).

### Preparation of 4-[2-[[(1R)-1-[2-(4,4-dimethyl-1-piperidyl)-3,6-dimethyl-4-oxo-chromen-8-yl]ethyl]amino]-6-fluoro-phenyl]-2-hydroxy-benzaldehyde

The title compound was prepared as a yellow solid (0.6 g, crude) according to the procedures described herein and the synthetic scheme, using 8-[(1R)-1-(2-bromo-3-fluoro-anilino)ethyl]-2-(4,4-dimethyl-1-piperidyl)-3,6-dimethyl-chromen-4-one (1.00 g, 1.99 mmol, 1 eq) and 2-hydroxy-4-(4,4,5,5-tetramethyl-1,3,2-dioxaborolan-2-yl)benzaldehyde (1.48 g, 5.98 mmol, 3 eq). The product was used directly for next step without further purification.

### Preparation of [5-[2-[[(1R)-1-[2-(4,4-dimethyl-1-piperidyl)-3,6-dimethyl-4-oxo-chromen-8-yl]ethyl]amino]-6-fluoro-phenyl]-2-formyl-phenyl]trifluoromethanesulfonate

The title compound was prepared as a yellow solid (1.20 g, 1.78 mmol, 96.51% yield) according to the procedures described herein and the synthetic scheme, using 4-[2-[[(1R)-1-[2-(4,4-dimethyl-1-piperidyl)-3,6-dimethyl-4-oxo-chromen-8-yl]ethyl]amino]-6-fluoro-phenyl]-2-hydroxy-benzaldehyde (1.00 g, 1.84 mmol, 1 eq) and PhNTf₂ (658 mg, 1.84 mmol, 1 eq). ¹H NMR (CDCl₃, 400 MHz) δ 10.35 (s, 1H), 8.17 (d, J=8.0 Hz, 1H), 7.87 (s, 1H), 7.67-7.65 (m, 1H), 7.60 (s, 1H), 7.12-7.06 (m, 1H), 6.54-6.50 (m, 1H), 6.19 (d, J=8.4 Hz, 1H), 5.01-4.96 (m, 1H), 4.08-4.06 (m, 1H), 3.39-3.36 (m, 4H), 2.36 (s, 3H), 2.06 (s, 3H), 1.52-1.47 (m, 7H), 1.03 (s, 6H).

### Preparation of 4-[2-[[(1R)-1-[2-(4,4-dimethyl-1-piperidyl)-3,6-dimethyl-4-oxo-chromen-8-yl]ethyl]amino]-6-fluoro-phenyl]-2-(4,4,5,5-tetramethyl-1,3,2-dioxaborolan-2-yl)benzaldehyde and 2-(4,4-dimethyl-1-piperidyl)-8-[(1R)-1-[3-fluoro-2-(1-hydroxy-3H-2,1-benzoxaborol-6-yl)anilino]ethyl]-3,6-dimethyl-chromen-4-one

The title compound was prepared as a yellow solid (1.2 g, crude) according to the procedures described herein and the synthetic scheme, using [5-[2-[[(1R)-1-[2-(4,4-dimethyl-1-piperidyl)-3,6-dimethyl-4-oxo-chromen-8-yl]ethyl]amino]-6-fluoro-phenyl]-2-formyl-phenyl]trifluoromethanesulfonate (1.00 g, 1.48 mmol, 1 eq) and B₂Pin₂ (1.88 g, 7.41 mmol, 5 eq). The product was used directly for next step without further purification. A by-product 2-(4,4-dimethyl-1-piperidyl)-8-[(1R)-1-[3-fluoro-2-(1-hydroxy-3H-2,1-benzoxaborol-6-yl)anilino]ethyl]-3,6-dimethyl-chromen-4-one was also isolated (200 mg) as a yellow solid. ¹H NMR (DMSO-d₆, 400 MHz) δ 9.25 (s, 1H), 7.69 (s, 1H), 7.61 (s, 1H), 7.53 (d, J=4.0 Hz, 1H), 7.44-7.42 (m, 1H), 7.35 (d, J=4.0 Hz, 1H), 7.08 (dd, J=8.0 Hz, 12.0 Hz, 1H), 6.50 (d, J=1.6 Hz, 1H), 6.32 (d, J=8.0 Hz, 1H), 5.05 (s, 2H), 4.99-4.92 (m, 1H), 4.64 (d, J=8.0 Hz, 1H), 3.31-3.25 (m, 4H), 2.32 (s, 3H), 1.90 (s, 3H), 1.43-1.41 (m, 7H), 0.96 (s, 6H). MS (ESI): mass calcd. For C₃₃H₃₆BFN₂O₄ 554.28, m/z found 555.3 [M+H]⁺. HPLC: 100.00% (220 nm), 100.00% (254 nm). Chiral purity: 100% ee.

### Preparation of 2-(4,4-dimethyl-1-piperidyl)-8-[(1R)-1-[3-fluoro-2-(1-hydroxy-2,3,1-benzoxazaborinin-7-yl)anilino]ethyl]-3,6-dimethyl-chromen-4-one

The title compound was prepared as a white solid (15.9 mg, 27.58 μmol, 3.60% yield) according to the procedures described herein and the synthetic scheme, using 4-[2-[[(1R)-1-[2-(4,4-dimethyl-1-piperidyl)-3,6-dimethyl-4-oxo-chromen-8-yl]ethyl]amino]-6-fluoro-phenyl]-2-(4,4,5,5-tetramethyl-1,3,2-dioxaborolan-2-yl)benzaldehyde (500 mg, 766 μmol, 1 eq) and hydroxylamine;hydrochloride (106 mg, 1.53 mmol, 2 eq). ¹H NMR (DMSO-d₆, 400 MHz) δ 9.42 (s, 1H), 8.71 (s, 1H), 8.05 (s, 1H), 7.86 (d, J=8.0 Hz, 1H), 7.79 (d, J=8.0 Hz, 1H), 7.60 (s, 1H), 7.39 (d, J=1.6 Hz, 1H), 7.12-7.08 (m, 1H), 6.52 (d, J=8.8 Hz, 1H), 7.36 (d, J=8.8 Hz, 1H), 4.96 (s, 2H), 3.31-3.30 (m, 4H), 2.32 (s, 3H), 1.90 (s, 3H), 1.42-1.40 (m, 7H), 0.96 (s, 6H). MS (ESI): mass calcd. For C₃₃H₃₅BFN₃O₄ 567.27, m/z found 568.3 [M+H]⁺. HPLC: 98.44% (220 nm), 97.31% (254 nm). Chiral purity: 98.38% ee.

### Example 41

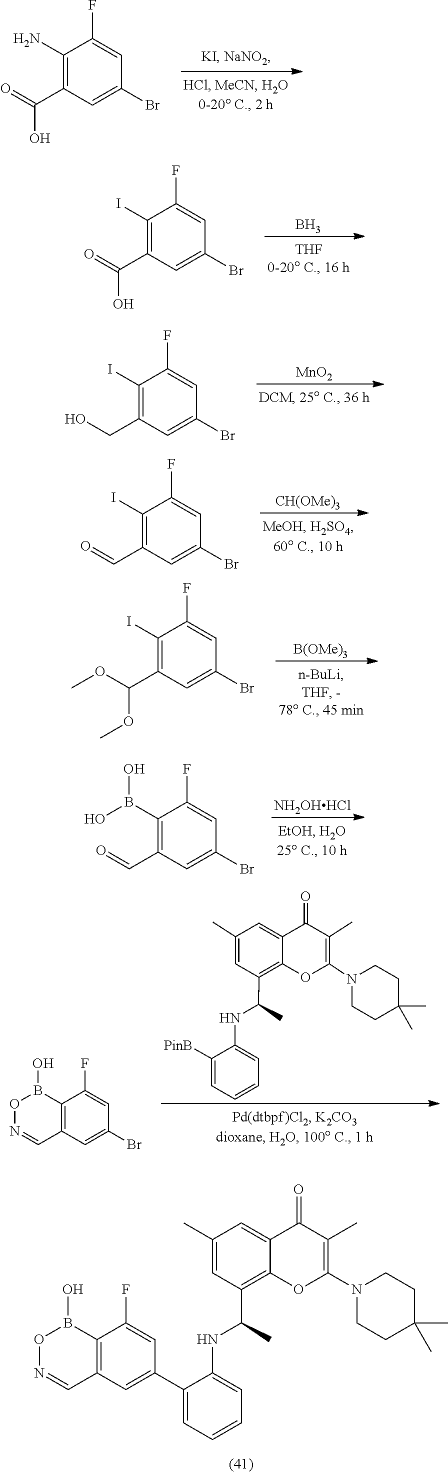

### Preparation of 5-bromo-3-fluoro-2-iodo-benzoic acid

To a solution of HCl (6 M, 56.9 mL, 10 eq) was added dropwise a solution of 2-amino-5-bromo-3-fluoro-benzoic acid (8.00 g, 34.1 mmol, 1 eq) in MeCN (40 mL) at 0° C. slowly over 10 min, the above mixture was stirred for 10 min. Then a solution of NaNO₂ (2.83 g, 41.0 mmol, 1.2 eq) in H₂O (10 mL) was added dropwise over 10 min, and the reaction mixture was stirred at 0° C. for 0.5 h. Finally a solution of KI (8.51 g, 51.2 mmol, 1.5 eq) in H₂O (10 mL) was added dropwise, the reaction mixture was slowly warmed to 20° C. and stirred at 20° C. for 2 h. The resulting suspension was filtered and the filter cake was washed with H₂O (30 mL). The filter cake was dissolved in EtOAc (200 mL)/H₂O (50 mL), adjusted to pH=3 with 6N HCl and extracted with EtOAc (200 mL×2). The combined organic layers were washed with brine, dried over Na₂SO₄, filtered and concentrated under reduced pressure to give the title compound (8.00 g, 23.1 mmol, 67.8% yield) as a yellow solid. ¹H NMR (DMSO-d₆, 400 MHz) δ 7.75 (dd, J=2.0, 7.6 Hz, 1H), 7.68 (s, 1H).

### Preparation of (5-bromo-3-fluoro-2-iodo-phenyl)methanol

To a solution of 5-bromo-3-fluoro-2-iodo-benzoic acid (8.00 g, 23.1 mmol, 1 eq) in THF (160 mL) was added BH₃-Me₂S (10 M, 11.6 mL, 5 eq) dropwise at 0° C., the reaction mixture was slowly warmed to 20° and stirred at 20° C. for 16 h. The reaction mixture was quenched by adding H₂O (50 mL) dropwise at 0° C., then diluted with H₂O (100 mL) and extracted with EtOAc (300 mL). The combined organic layers were washed with brine, dried over Na₂SO₄, filtered and concentrated under reduced pressure to give a residue. The residue was purified by flash silica gel chromatography (BIOTAGE®; 80 g SepaFlash® Silica Flash Column, Eluent of 0~50% Ethyl acetate/Petroleum ether gradient @100 mL/min) to give the title compound (7.30 g, 22.0 mmol, 95.1% yield) as a yellow solid. ¹H NMR (DMSO-d₆, 400 MHz) δ 7.49 (dd, J=2.0, 7.6 Hz, 1H), 7.44 (d, J=1.2 Hz, 1H), 4.43 (s, 2H).

### Preparation of 5-bromo-3-fluoro-2-iodo-benzaldehyde

To a solution of (5-bromo-3-fluoro-2-iodo-phenyl)methanol (7.30 g, 22.0 mmol, 1 eq) in DCM (500 mL) was added MnO₂ (19.1 g, 220 mmol, 10 eq) portion-wise at 25° C., the reaction mixture was stirred at 25° C. for 36 h. The reaction mixture was filtered and the filtrate was concentrated under reduced pressure to give a residue. The residue was purified by flash silica gel chromatography (BIOTAGE®; 120 g SepaFlash® Silica Flash Column, Eluent of 0~30% Ethyl acetate/Petroleum ether gradient @100 mL/min) to give the title compound (6.40 g, 19.4 mmol, 88.2% yield) as a yellow solid. ¹H NMR (CDCl₃, 400 MHz) δ 10.06 (s, 1H), 7.83 (dd, J=1.2, 2.0 Hz, 1H), 7.48 (dd, J=2.4, 7.2 Hz, 1H).

### Preparation of 5-bromo-1-(dimethoxymethyl)-3-fluoro-2-iodo-benzene

To a solution of 5-bromo-3-fluoro-2-iodo-benzaldehyde (2.00 g, 6.08 mmol, 1 eq) and trimethoxymethane (1.29 g, 12.1 mmol, 1.33 mL, 2 eq) in MeOH (30 mL) was added H₂SO₄ (60.8 mg, 608 μmol, 33.0 μL, 0.1 eq) at 25° C. The reaction mixture was heated to 60° C. and stirred at 60° C. for 10 h. The reaction mixture was cooled to 0° C., quenched with sat. aq NaHCO₃ (50 mL) and extracted with EtOAc (60 mL×3). The combined organic layers were washed with brine (60 mL), dried over Na₂SO₄, filtered and concentrated under reduced pressure to give the title compound (2.28 g, 6.08 mmol, 100.00% yield) as yellow oil. ¹H NMR (DMSO-d₆, 400 MHz) δ 7.64 (dd, J=2.0, 7.6 Hz, 1H), 7.41 (d, J=1.2 Hz, 1H), 5.35 (s, 1H), 3.32 (s, 6H).

### Preparation of (4-bromo-2-fluoro-6-formyl-phenyl)boronic acid

To a solution of 5-bromo-1-(dimethoxymethyl)-3-fluoro-2-iodo-benzene (3.00 g, 8.0 mmol, 1 eq) in THF (30 mL) was added n-BuLi (2.5 M, 3.52 mL, 1.1 eq) dropwise at −78° C., the reaction mixture was stirred at −78° C. for 15 min. Then trimethyl borate (914 mg, 8.80 mmol, 994 μL, 1.1 eq) was added to the reaction mixture dropwise at −78° C. and the resulting reaction mixture was stirred at −78° C. for 30 min. The mixture was quenched with HCl (10 mL, 1 M) slowly, stirred for 15 min and extracted with EtOAc (100 mL×3). The combined organic phase was washed with brine (100 mL), dried over Na₂SO₄, filtered and concentrated in vacuum to give the title compound (2.00 g, crude) as yellow oil.

### Preparation of 6-bromo-8-fluoro-1-hydroxy-2,3,1-benzoxazaborinine

To a solution of (4-bromo-2-fluoro-6-formyl-phenyl)boronic acid (2.00 g, 8.10 mmol, 1 eq) in EtOH (20 mL) and H₂O (2.5 mL) was added hydroxylamine;hydrochloride (1.69 g, 24.3 mmol, 3 eq) in one portion at 25° C., the reaction mixture was stirred at 25° C. for 10 h. The reaction mixture was directly concentrated in vacuo to remove the solvent. Then the residue was suspended in MeCN (20 mL), filtered and the filtrate was concentrated in vacuo to give a residue. The residue was triturated with EtOH (5 ml)/H₂O (2 ml) and filtered. The filter cake was washed with MeCN (2 mL) and dried in vacuo to give the title compound (0.90 g, 3.69 mmol, 45.5% yield) as a yellow solid. ¹H NMR (DMSO-d₆, 400 MHz) δ 8.64 (d, J=2.4 Hz, 1H), 7.87 (s, 1H), 7.81 (d, J=8.4 Hz, 1H).

### Preparation of 2-(4,4-dimethyl-1-piperidyl)-8-[(1R)-1-[2-(8-fluoro-1-hydroxy-2,3,1-benzoxazaborinin-6-yl)anilino]ethyl]-3,6-dimethyl-chromen-4-one

A mixture of 6-bromo-8-fluoro-1-hydroxy-2,3,1-benzoxazaborinine (91.9 mg, 377 μmol, 1 eq) and 2-(4,4-dimethyl-1-piperidyl)-3,6-dimethyl-8-[(1R)-1-[2-(4,4,5,5-tetramethyl-1,3,2-dioxabo rolan-2-yl)anilino]ethyl]chromen-4-one (200 mg, 377 μmol, 1 eq) in dioxane (2 mL) and H₂O (0.3 mL) were added K₂CO₃ (104 mg, 754 μmol, 2 eq) and ditert-butyl(cyclopentyl)phosphane;dichloropalladium;iron (24.5 mg, 37.7 μmol, 0.1 eq) in turns at 20° C., the reaction was degassed and purged with N₂ for 3 times and stirred at 100° C. (the oil-bath was pre-heated to 100° C.) for 1 h under N₂ atmosphere. The reaction mixture was cooled to 20° C., filtered and the filtrate was directly purified by prep-HPLC (column: Waters Xbridge Prep OBD C18 150×40 mm×10 um; mobile phase: [H2O (10 mM □H4HCO3)-AC□]; gradient:25%-55% B over 8.0 min) to give the title compound (37.7 mg, 66.4 μmol, 17.6% yield) as an off-white solid. ¹H NMR (DMSO-d₆, 400 MHz) δ 9.14 (s, 1H), 8.65 (s, 1H), 7.67 (s, 1H), 7.60 (s, 1H), 7.56 (d, J=9.6 Hz, 1H), 7.48 (s, 1H), 7.14-7.08 (m, 1H), 7.08-7.00 (m, 1H), 6.76-6.63 (m, 1H), 6.53 (d, J=8.0 Hz, 1H), 5.26 (d, J=6.4 Hz, 1H), 4.93 (t, J=6.4 Hz, 1H), 3.32-3.30 (m, 4H), 2.32 (s, 3H), 1.90 (s, 3H), 1.47 (d, J=6.4 Hz, 3H), 1.43-1.38 (m, 4H), 0.96 (s, 6H). MS (ESI): mass calcd. For C₃₃H₃₅BFN₃O₄ 567.27, m/z found 568.3 [M+H]⁺. HPLC: 98.54% (220 nm), 98.19% (254 nm). Chiral purity: 100 ee %

### Example 42

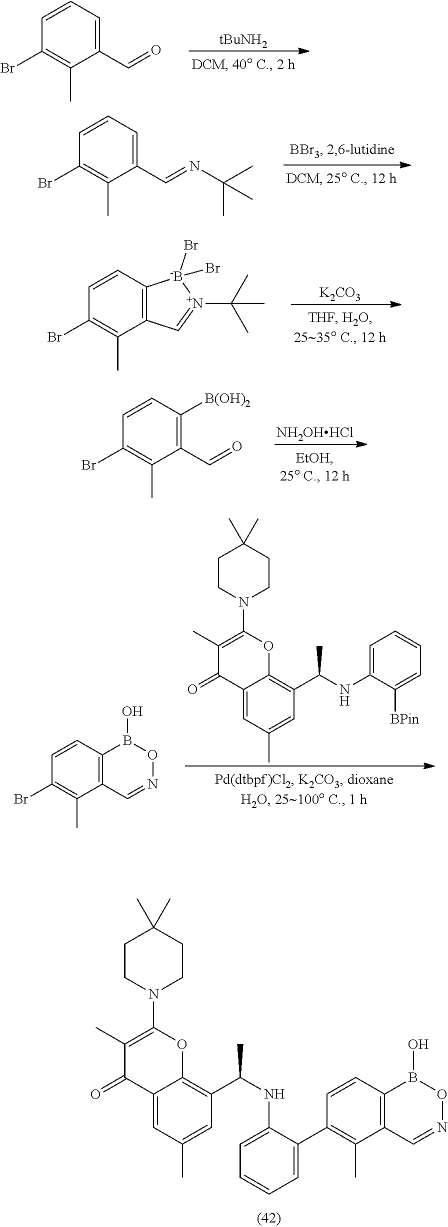

### Preparation of (E)-1-(3-bromo-2-methylphenyl)-N-(tert-butyl)methanimine

A solution of 2-methylpropan-2-amine (7.35 g, 100.5 mmol, 10.6 mL, 4 eq) and 3-bromo-2-methylbenzaldehyde (5.00 g, 25.1 mmol, 1 eq) in DCM (200 mL) was stirred at 40° C. for 2 h. The reaction mixture was cooled to 25° C. and concentrated in vacuo directly to give the title compound (6.40 g, crude) as an off-white solid, which was used directly for next step without further purification.

### Preparation of 3,7,7-tribromo-8-tert-butyl-2-methyl-8-azonia-7-boranuidabicyclo[4.3.0]nona-1(6),2,4,8-tetraene

To a solution of (E)-1-(3-bromo-2-methyl-phenyl)-N-tert-butyl-methanimine (3.00 g, 11.8 mmol, 1 eq) and 2,6-lutidine (2.50 g, 23.6 mmol, 2.7 mL, 2 eq) in DCM (30 mL) was added BBr₃ (5.90 g, 23.6 mmol, 2.3 mL, 2 eq) dropwise at 25° C. under N₂ atmosphere, the reaction mixture was stirred at 25° C. for 12 h. The reaction mixture was poured into H₂O (50 mL) at 0° C. and then extracted with DCM (50 mL×3). The combined organic layers were washed with brine, dried over Na₂SO₄, filtered and concentrated under reduced pressure to give the title compound (3.17 g, 63.38% yield) as a light yellow solid. ¹H NMR (DMSO-d₆, 400 MHz) δ 9.40 (s, 1H), 7.97 (d, J=7.6 Hz, 1H), 7.88 (d, J=7.6 Hz, 1H), 2.64 (s, 3H), 1.54 (s, 9H).

### Preparation of (4-bromo-2-formyl-3-methyl-phenyl)boronic acid

To a solution of 3,7,7-tribromo-8-tert-butyl-2-methyl-8-azonia-7-boranuidabicyclo[4.3.0]nona-1(6),2,4,8-tetraene (3.17 g, 7.48 mmol, 1 eq) in THF (60 mL)/H₂O (60 mL) was added K₂CO₃ (10.30 g, 74.8 mmol, 10 eq) in portions, the reaction mixture was heated to 35° C. and stirred at 35° C. for 12 h. The reaction mixture was cooled to 25° C., adjusted pH to 2-3 with 2N HCl and then extracted with EtOAc (150 mL×2). The combined organic layers were washed with brine (100 mL), dried over anhydrous Na₂SO₄, filtered and concentrated under reduced pressure to give a residue. The residue was triturated with ACN (50 mL), filtered and the filter cake was dried in vacuo to give the title compound (2.80 g, crude) as a light yellow solid.

### Preparation of 6-bromo-1-hydroxy-5-methyl-2,3,1-benzoxazaborinine

To a solution of (4-bromo-2-formyl-3-methyl-phenyl)boronic acid (900 mg, 3.71 mmol, 1 eq) in EtOH (10 mL) was added hydroxylamine hydrochloride (515 mg, 7.41 mmol, 2 eq) in one portion at 25° C. under N₂ atmosphere, the reaction mixture was stirred at 25° C. for 12 h. The reaction mixture was directly concentrated under reduced pressure to remove the solvent and the residue was triturated with H₂O (10 mL). The resulting suspension was filtered and the filter cake was dried in vacuo to give the title compound (800 mg, 3.34 mmol, 90.0% yield) as a white solid. ¹H NMR (DMSO-d₆, 400 MHz) δ 9.46 (s, 1H), 8.89 (s, 1H), 7.95 (d, J=8.0 Hz, 1H), 7.80 (d, J=8.0 Hz, 1H), 2.63 (s, 3H).

### Preparation of 2-(4,4-dimethyl-1-piperidyl)-8-[(R)-1-[2-(1-hydroxy-5-methyl-2,3,1-ben zoxazaborinin-6-yl)anilino]ethyl]-3,6-dimethyl-chromen-4-one

To a solution of 2-(4,4-dimethyl-1-piperidyl)-3,6-dimethyl-8-[(1R)-1-[2-(4,4,5,5-tetramethyl-1, 3,2-dioxaborolan-2-yl)anilino]ethyl]chromen-4-one (200 mg, 377 μmol, 1 eq) 6-bromo-1-hydroxy-5-methyl-2,3,1-benzoxazaborinine (90 mg, 377 μmol, 1 eq) and K₂CO₃ (104 mg, 754 μmol, 2 eq) in dioxane (2 mL)/H₂O (0.3 mL) was added Pd(dtbpf)Cl₂ (25 mg, 37.7 μmol, 0.1 eq) in one portion at 25° C. under N₂ atmosphere, the reaction mixture was heated to 100° C. and stirred at 100° C. for 1 h. 3 parallel reactions were combined for work up. The reaction mixture was cooled to 25° C., filtered and the filtrate was directly purified by prep-HPLC (column: Waters Xbridge BEH C18 100×30 mm×10 um; mobile phase: [H₂O (10 mM NH₄HCO₃)-AC□]; gradient:65%-95% B over 8.0 min) to give the title compound (29 mg) as an off-white solid. ¹H NMR (DMSO-d₆, 400 MHz) δ 9.34 (d, J=8.4 Hz, 1H), 8.82 (d, J=8.8 Hz, 1H), 7.97 (d, J=7.6 Hz, 1H), 7.61-7.60 (m, 1H), 7.57-7.30 (m, 3H), 6.90-6.80 (m, 1H), 6.67-6.57 (m, 2H), 4.94 (t, J=6.8 Hz, 1H), 4.47 (dd, J=46.8 Hz, 7.6 Hz, 1H), 3.29-3.22 (m, 4H), 2.29-2.24 (m, 6H), 1.90 (s, 3H), 1.41 (t, J=6.4 Hz, 7H), 0.96 (s, 6H). MS (ESI): mass calcd. For C₃₄H₃₈BN₃O₄ 563.51, m/z found 564.3 [M+H]⁺. HPLC: 98.56 (220 nm), 99.34 (240 nm). Chiral purity: 100 ee %.

### Example 43

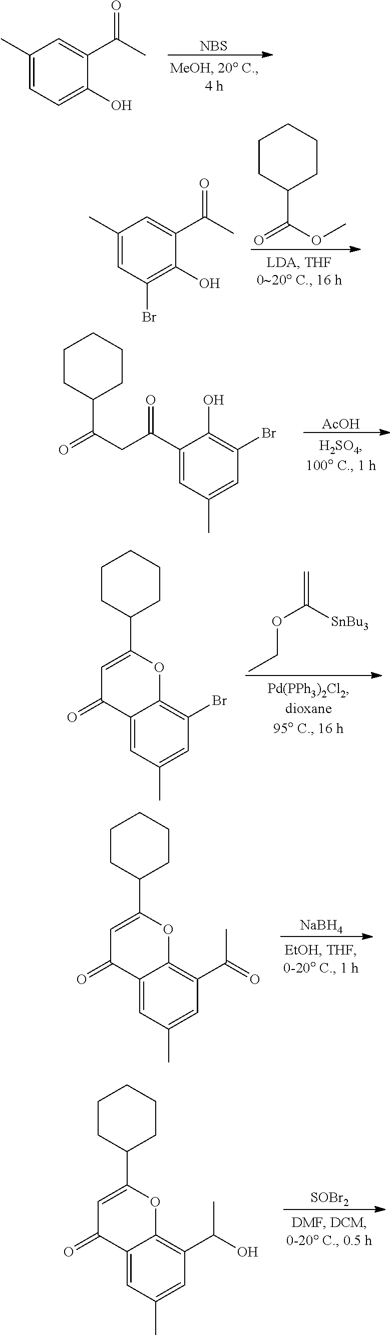

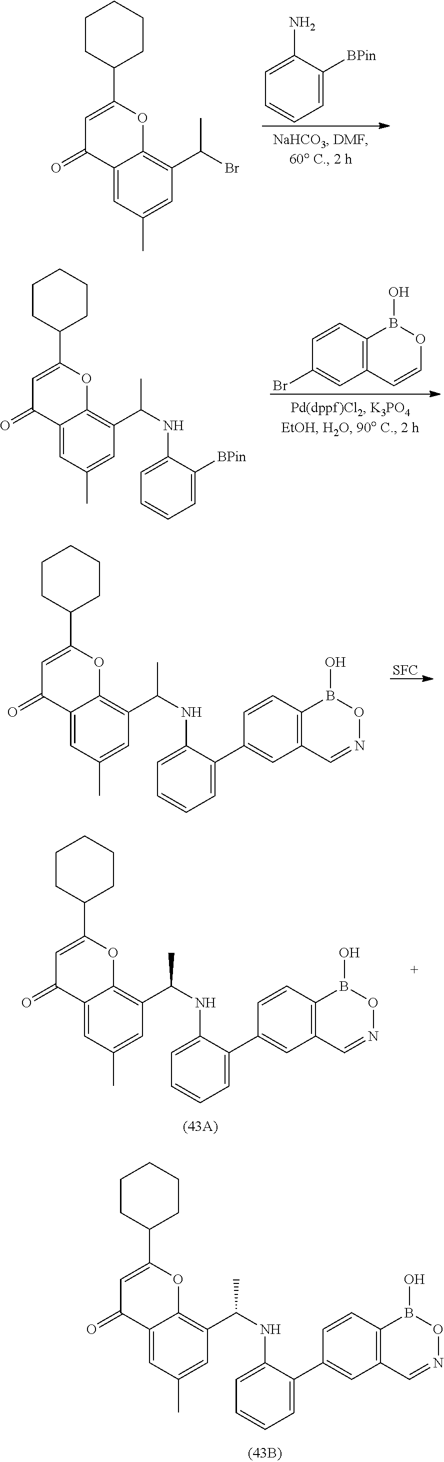

### Preparation of 1-(3-bromo-2-hydroxy-5-methyl-phenyl)ethanone

To a solution of 1-(2-hydroxy-5-methyl-phenyl)ethanone (15.0 g, 99.9 mmol, 1 eq) in MeOH (100 mL) was added NBS (26.7 g, 150 mmol, 1.5 eq) in portions at 20° C. Then the reaction mixture was stirred at 20° C. for 4 h. The reaction mixture was poured into H₂O (150 mL) at 0° C. and some solid formed. The reaction mixture was filtered under reduced pressure to collect the filter cake to give the title compound (22.0 g, 96.0 mmol, 96.15% yield) as a yellow solid. ¹H NMR (DMSO-d₆, 400 MHz) δ 12.63 (m, 1H), 7.79 (s, 1H), 7.73 (s, 1H), 2.67 (s, 3H), 2.29 (s, 3H).

### Preparation of 1-(3-bromo-2-hydroxy-5-methyl-phenyl)-3-cyclohexyl-propane-1,3-dione

To a solution of 1-(3-bromo-2-hydroxy-5-methyl-phenyl)ethanone (30.0 g, 131 mmol, 1 eq) in THF (200 mL) was added LDA (2 M in THF, 196.45 mL, 3 eq) dropwise at 0° C. After stirring at 0° C. for 1.5 h, a solution of methyl cyclohexanecarboxylate (37.2 g, 262 mmol, 2 eq) in THF (50 mL) was added dropwise to the reaction mixture at 0° C. Then the reaction mixture was warmed to 20° C. and stirred at 20° C. for 16 h. The reaction mixture was poured into ice-water (w/w=1/1) (300 mL) and extracted with EtOAc (200 mL×3). The combined organic phase was washed with brine (200 mL), dried with anhydrous Na₂SO₄, filtered and concentrated in vacuo. The residue was purified by flash silica gel chromatography (ISCO®; 220 g SepaFlash® Silica Flash Column, Eluent of 0~10% Ethyl acetate/Petroleum ether gradient@150 mL/min) to give the title compound (21.0 g, crude) as yellow oil, which was used directly for next step without further purification.

### Preparation of 8-bromo-2-cyclohexyl-6-methyl-chromen-4-one

To a solution of 1-(3-bromo-2-hydroxy-5-methyl-phenyl)-3-cyclohexyl-propane-1,3-dione (10.0 g, 29.48 mmol, 1 eq) in AcOH (50 mL) was added H₂SO₄ (10 mL) at 20° C. dropwise. Then the reaction mixture was heated to 100° C. and stirred at 100° C. for 1 h. The reaction mixture was cooled to 25° C., poured into ice water (200 mL) and extracted with EtOAc (200 mL×3). The combined organic phase was washed with brine (100 mL), dried with anhydrous Na₂SO₄, filtered and concentrated in vacuo. The residue was purified by flash silica gel chromatography (ISCO®; 120 g SepaFlash® Silica Flash Column, Eluent of 0~10% Ethyl acetate/Petroleum ether gradient@100 mL/min) to give the title compound (5.0 g, 15.57 mmol, 52.80% yield) as yellow oil. ¹H NMR (CDCl₃, 400 MHz) δ 7.92 (s, 1H), 7.71 (s, 1H), 6.19 (s, 1H), 2.61-2.60 (m, 1H), 2.43 (s, 3H), 2.11-1.40 (m, 10H).

### Preparation of 8-acetyl-2-cyclohexyl-6-methyl-chromen-4-one

To a solution of 8-bromo-2-cyclohexyl-6-methyl-chromen-4-one (5.00 g, 15.5 mmol, 1 eq) and tributyl(1-ethoxyvinyl)stannane (9.41 g, 26.1 mmol, 8.80 mL, 1.67 eq) in dioxane (100 mL) was added Pd(PPh₃)₂Cl₂ (546 mg, 778 μmol, 0.05 eq) in one portion at 20° C. The reaction mixture was degassed and purged with N₂ for 3 times and stirred at 95° C. for 16 h under N₂ atmosphere. The reaction mixture was cooled to 20° C., added HCl (30 mL, 2 M) and stirred at 50° C. for 0.5 h. The reaction mixture was cooled to 20° C., added sat. aq. KF (200 mL), stirred for 0.5 h and then filtered. The filter cake was washed with EtOAc (250 mL×3) and the filtrate was extracted with EtOAc (250 mL×3). The combined organic phase was washed with brine (250 mL), dried over Na₂SO₄, filtered and concentrated under reduced pressure. The residue was purified by flash silica gel chromatography (ISCO®; 120 g SepaFlash® Silica Flash Column, Eluent of 0~30% Ethyl acetate/Petroleum ether gradient@100 mL/min) to give the title compound (4.50 g, crude) as yellow solid. ¹H NMR (DMSO-d₆, 400 MHz) δ 8.17 (d, J=2.4 Hz, 1H), 7.90 (d, J=2.4 Hz, 1H), 6.22 (s, 1H), 2.78 (s, 3H), 2.59-2.57 (m, 1H), 2.47 (s, 3H), 2.08-2.05 (m, 2H), 1.94-1.89 (m, 2H), 1.54-1.26 (m, 6H).

### Preparation of 2-cyclohexyl-8-(1-hydroxyethyl)-6-methyl-4H-chromen-4-one

To s solution of 8-acetyl-2-cyclohexyl-6-methyl-chromen-4-one (3.5 g, 12.31 mmol, 1 eq) in EtOH (5 mL)/THF (30 mL) was added NaBH₄ (0.68 g, 17.98 mmol, 1.46 eq) at 0° C. in portions. Then the reaction mixture was stirred at 20° C. for 1 h. The residue was poured into ice-water (w/w=1/1) (50 mL) and extracted with DCM (30 mL×3). The combined organic phase was washed with brine (20 mL), dried with anhydrous Na₂SO₄, filtered and concentrated in vacuum to give the title compound (4.0 g, crude) as yellow solid. ¹H NMR (DMSO-d₆, 400 MHz) δ 7.89 (d, J=1.2 Hz, 1H), 7.64 (d, J=2.4 Hz, 1H), 6.16 (s, 1H), 5.49-5.43 (m, 1H), 2.58-2.55 (m, 1H), 2.45 (s, 3H), 2.06-2.02 (m, 3H), 1.91-1.90 (m, 2H), 1.89-1.88 (m, 1H), 1.46 (d, J=9.6 Hz, 1H), 1.43-1.26 (m, 6H).

### Preparation of 8-(1-bromoethyl)-2-cyclohexyl-6-methyl-4H-chromen-4-one

To a solution of 2-cyclohexyl-8-(1-hydroxyethyl)-6-methyl-4H-chromen-4-one (2.50 g, 8.73 mmol, 1 eq) in DCM (10 mL)/DMF (63 mg, 873.01 μmol, 67 μL, 0.1 eq) was added SOBr₂ (2.72 g, 13.1 mmol, 1.01 mL, 1.5 eq) at 0° C. The reaction mixture was warmed to 20° C. and stirred at 20° C. for 0.5 h.

The reaction mixture was quenched by H₂O (20 mL) at 0° C., and then extracted with DCM (30 mL×2).

The combined organic layers were washed with brine, dried over anhydrous Na₂SO₄, filtered and concentrated under reduced pressure to give the title compound (2.50 g, 7.16 mmol, 81.99% yield) as a yellow solid. ¹H NMR (CDCl₃, 400 MHz) δ 7.94-7.94 (m, 1H), 7.62-7.62 (m, 1H), 6.19 (s, 1H), 5.72 (q, J=7.2 Hz, 1H), 2.60-2.59 (m, 1H), 2.45 (s, 3H), 2.16 (s, 3H), 2.16-2.94 (m, 1H), 1.92-1.89 (m, 2H), 1.58-1.53 (m, 1H), 1.44-1.41 (m, 3H), 1.28-1.26 (m, 3H).

### Preparation of 2-cyclohexyl-6-methyl-8-(1-((2-(4,4,5,5-tetramethyl-1,3,2-dioxaborolan-2-yl)phenyl)amino)ethyl)-4H-chromen-4-one

A mixture of 8-(1-bromoethyl)-2-cyclohexyl-6-methyl-4H-chromen-4-one (1.00 g, 2.86 mmol, 1 eq), 2-(4,4,5,5-tetramethyl-1,3,2-dioxaborolan-2-yl)aniline (1.25 g, 5.73 mmol, 2 eq) and NaHCO₃ (481 mg, 5.73 mmol, 222 μL, 2 eq) in DMF (10 mL) was degassed and purged with N₂ for 3 times at 20° C., and then the mixture was heated to 60° C. stirred at 60° C. for 2 h under N₂ atmosphere. The reaction mixture was quenched by adding water (40 mL) at 0° C. and the resulting suspension was filtered. The filter cake was washed with H₂O (5 mL×3) and dried in vacuo to the title compound (750 mg, 1.54 mmol, 53.63% yield) as a yellow solid. ¹H NMR (DMSO-d₆, 400 MHz) δ 7.67 (s, 1H), 7.49 (s, 1H), 7.44-7.42 (m, 1H), 7.15-7.11 (m, 1H), 6.52 (t, J=7.6 Hz, 1H), 6.33-6.30 (m, 2H), 6.19 (s, 1H), 5.12-5.06 (m, 1H), 2.65-2.64 (m, 1H), 2.33 (s, 3H), 1.98-1.95 (m, 2H), 1.79-1.78 (m, 2H), 1.77-1.77 (m, 1H), 1.55-1.53 (m, 3H), 1.48-1.48 (m, 2H), 1.32-1.31 (m, 1H), 1.22 (s, 12H), 1.22-1.16 (m, 2H).

### Preparation of 2-cyclohexyl-8-(1-((2-(1-hydroxy-1H-benzo[d][1,2,6]oxazaborinin-6-yl) phenyl)amino)ethyl)-6-methyl-4H-chromen-4-one

A mixture of 2-cyclohexyl-6-methyl-8-(1-((2-(4,4,5,5-tetramethyl-1,3,2-dioxaborolan-2-yl)phenyl)amino) ethyl)-4H-chromen-4-one (150 mg, 307 μmol, 1 eq), 6-bromo-1H-benzo [c][1,2]oxaborinin-1-ol (104 mg, 461 μmol, 1.5 eq), K₃PO₄ (130 mg, 615 μmol, 2 eq) and Pd(dppf)Cl₂ (22 mg, 30.7 μmol, 0.1 eq) in H₂O (0.1 mL)/EtOH (0.5 mL) was degassed and purged with N₂ for 3 times at 20° C., and then the mixture was heated to 90° C. and stirred at 90° C. for 2 h under N₂ atmosphere. The reaction mixture was filtered and the filtrate was concentrated under reduced pressure to give a residue. The residue was purified by prep-HPLC (column: Phenomenex Luna C18 75×30 mm×3 um; mobile phase: [H2O (0.2% FA)-AC□]; gradient:50%-90% B over 8.0 min) to give the title compound (60 mg, 117.24 μmol, 38.10% yield) as an off-white solid. ¹H NMR (DMSO-d₆, 400 MHz) δ 9.43-9.36 (m, 1H), 8.66 (s, 1H), 8.13 (d, J=7.6 Hz, 1H), 7.84-7.87 (m, 2H), 7.62 (d, J=9.6 Hz, 2H), 7.10-7.03 (m, 2H), 6.71 (t, J=7.2 Hz, 1H), 6.61-6.59 (m, 1H), 6.12 (s, 1H), 5.09-5.07 (m, 1H), 5.02-4.95 (m, 1H), 2.34-2.34 (m, 4H), 1.86-1.83 (m, 2H), 1.83-1.71 (m, 2H), 1.46-1.44 (m, 3H), 1.34-1.10 (m, 6H). MS (ESI): mass calcd. For C₃₁H₃₁BN₂O₄ 506.41, m/z found 507.3 [M+H]⁺. HPLC: 98.95% (220 nm), 99.82% (254 nm).

### Preparation of (R)-2-cyclohexyl-8-(1-((2-(1-hydroxy-1H-benzo [d][1,2,6]oxazaborinin-6-yl)phenyl)amino)ethyl)-6-methyl-4H-chromen-4-one & (S)-2-cyclohexyl-8-(1-((2-(1-hydroxy-1H-benzo[d][1,2,6]oxazaborinin-6-yl)phenyl)amino)ethyl)-6-methyl-4H-chromen-4-one

2-cyclohexyl-8-(1-((2-(1-hydroxy-1H-benzo[d][1,2,6]oxazaborinin-6-yl)phenyl)amino)ethyl)-6-methyl-4H-chromen-4-one (60 mg, 118 μmol, 1 eq) was separated by SFC (column: DAICEL CHIRALCEL OD (250 mm×30 mm, 10 um); mobile phase: [CO2-IPA (0.1% □H3H2O)]; B %: 50%, isocratic elution mode) to give the title compound, Isomer 1 (30.9 mg, 60.30 μmol, 50.89% yield) as a white solid and the title compound, Isomer 2 (28.7 mg, 56.60 μmol, 47.77% yield) as a white solid. Isomer 1: ¹H NMR (DMSO-d₆, 400 MHz) δ 9.55-9.36 (m, 1H), 8.61-8.47 (m, 1H), 8.06-8.04 (m, 1H), 7.75-7.54 (m, 4H), 7.08-7.02 (m, 2H), 6.71-6.57 (m, 2H), 6.13 (s, 1H), 5.09-4.81 (m, 2H), 2.34 (s, 3H), 1.88-1.86 (m, 2H), 1.72-1.65 (m, 3H), 1.46-1.44 (m, 3H), 1.40-1.14 (m, 6H). MS (ESI): mass calcd. For C₃₁H₃₁BN₂O₄ 506.41, m/z found 507.3 [M+H]⁺. HPLC: 98.82% (220 nm), 98.95% (254 nm). Chiral purity: 100% ee. Isomer 2: ¹H NMR (DMSO-d₆, 400 MHz) δ 9.44-9.38 (m, 1H), 8.63-8.61 (m, 1H), 8.12-8.12 (m, 1H), 7.80-7.53 (m, 4H), 7.09-7.03 (m, 2H), 6.72-6.46 (m, 2H), 6.13 (s, 1H), 5.01-4.76 (m, 2H), 2.34 (s, 3H), 1.87-1.84 (m, 2H), 1.75-1.72 (m, 3H), 1.46-1.44 (m, 3H), 1.35-1.14 (m, 6H). MS (ESI): mass calcd. For C₃₁H₃₁BN₂O₄ 506.41, m/z found 507.3 [M+H]⁺. HPLC: 99.86% (220 nm), 99.87% (254 nm). Chiral purity: 99.38% ee.

### Example 44

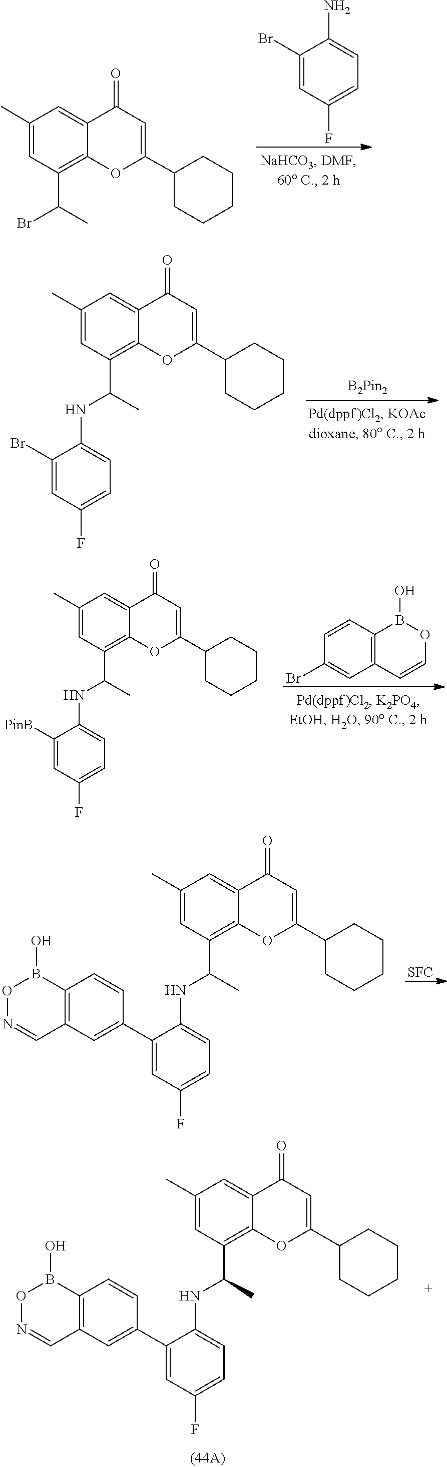

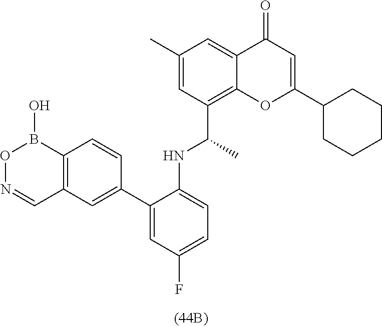 [SMILES: Cc1cc([C@H](C)Nc2ccc(F)cc2-c2ccc3c(c2)C=NOB3O)c2oc(C3CCCCC3)cc(=O)c2c1]

### Preparation of 8-(1-((2-bromo-4-fluorophenyl)amino)ethyl)-2-cyclohexyl-6-methyl-4H-chromen-4-one

A mixture of 8-(1-bromoethyl)-2-cyclohexyl-6-methyl-4H-chromen-4-one (1.00 g, 2.86 mmol, 1 eq), 2-bromo-4-fluoroaniline (1.09 g, 5.73 mmol, 2 eq) and NaHCO₃ (481 mg, 5.73 mmol, 2 eq) in DMF (10 mL) was degassed and purged with N₂ for 3 times at 20° C., and then the reaction mixture was heated to 60° C. stirred at 60° C. for 2 h under N₂ atmosphere. The reaction mixture was quenched by H₂O (30 mL) at 0° C., and then extracted with EtOAc (30 mL×3). The combined organic layers were washed with brine, dried over anhydrous Na₂SO₄, filtered and concentrated under reduced pressure to give the title compound (2.00 g, crude) as a yellow solid, which was used directly for next step without further purification. ¹H NMR (CDCl₃, 400 MHz) δ 7.95 (s, 1H), 7.67-7.66 (m, 1H), 7.58-7.57 (m, 1H), 7.43-7.40 (m, 1H), 6.44-6.41 (m, 1H), 6.19 (s, 1H), 5.24-5.22 (m, 1H), 5.09-5.02 (m, 1H), 2.34 (s, 3H), 2.05-2.04 (m, 1H), 1.82-1.78 (m, 4H), 1.60 (d, J=6.4 Hz, 3H), 1.50-1.34 (m, 6H).

### Preparation of 2-cyclohexyl-8-(1-((4-fluoro-2-(4,4,5,5-tetramethyl-1,3,2-dioxaborolan-2-yl)phenyl)amino)ethyl)-6-methyl-4H-chromen-4-one

A mixture of 8-(1-((2-bromo-4-fluorophenyl)amino)ethyl)-2-cyclohexyl-6-methyl-4H-chromen-4-one (1.00 g, 2.18 mmol, 1 eq), B₂Pin₂ (1.11 g, 4.36 mmol, 2 eq), Pd(dppf)Cl₂ (159 mg, 218 μmol, 0.1 eq) and KOAc (428 mg, 4.36 mmol, 2 eq) in dioxane (20 ml) was degassed and purged with N₂ for 3 times at 20° C., and then the mixture was heated to 80° C. and stirred at 80° C. for 2 h under N₂ atmosphere. 2 parallel reactions were combined for work up. The reaction mixture was cooled to 20° C., filtered and the filtrate was concentrated under reduced pressure to give a residue. The residue was purified by flash silica gel chromatography (Biotage®; 20 g SepaFlash® Silica Flash Column, Eluent of 10~30% Ethyl acetate/Petroleum ether gradient@75 mL/min) to give the title compound (1.10 g, crude) as brown oil, which was used directly for next step without further purification.

### Preparation of 2-cyclohexyl-8-(1-((4-fluoro-2-(1-hydroxy-1H-benzo[d][1,2,6]oxazaborinin-6-yl)phenyl)amino)ethyl)-6-methyl-4H-chromen-4-one

A mixture of 2-cyclohexyl-8-(1-((4-fluoro-2-(4,4,5,5-tetramethyl-1,3,2-dioxaborolan-2-yl) phenyl)amino)ethyl)-6-methyl-4H-chromen-4-one (500 mg, 989 μmol, 1 eq), 6-bromo-1H-benzo[d][1,2,6]oxazaborinin-1-ol (335 mg, 1.48 mmol, 1.5 eq), K₃PO₄ (419 mg, 1.98 mmol, 2 eq) and Pd(dppf)Cl₂ (72 mg, 98 μmol, 0.1 eq) in H₂O (1 mL)/EtOH (5 mL) was degassed and purged with N₂ for 3 times at 20° C., and then the mixture was heated to 90° C. and stirred at 90° C. for 2 h under N2 atmosphere. The reaction mixture was cooled to 20° C., filtered and the filtrate was concentrated under reduced pressure to give a residue. The residue was purified by prep-HPLC (column: Phenomenex Luna C18 100×30 mm×3 um; mobile phase: [1H₂O (0.2% FA)-AC□]; gradient:55%-95% B over 8.0 min) to give the title compound (100 mg, 190.70 μmol, 19.28% yield) as a white solid. ¹H NMR (DMSO-d₆, 400 MHz) δ 9.30-9.10 (m, 1H), 8.60 (s, 1H), 8.10 (d, J=7.6 Hz, 1H), 7.84-7.81 (m, 2H), 7.63 (s, 1H), 7.59 (s, 1H), 6.97-6.92 (m, 2H), 6.61-6.58 (m, 1H), 6.11 (s, 1H), 5.03-5.01 (m, 1H), 4.93-4.87 (m, 1H), 2.34-2.32 (m, 4H), 1.85-1.82 (m, 2H), 1.74-1.72 (m, 2H), 1.43-1.42 (m, 1H), 1.34-1.30 (m, 3H), 1.28-1.08 (m, 5H).

### Preparation of (R)-2-cyclohexyl-8-(1-((4-fluoro-2-(1-hydroxy-1H-benzo [d][1,2,6]oxazaborinin-6-yl)phenyl)amino)ethyl)-6-methyl-4H-chromen-4-one and (S)-2-cyclohexyl-8-(1-((4-fluoro-2-(1-hydroxy-1H-benzo[d][1,2,6]oxazaborinin-6-yl)phenyl)amino)ethyl)-6-methyl-4H-chromen-4-one

2-cyclohexyl-8-(1-((4-fluoro-2-(1-hydroxy-1H-benzo[d][1,2,6]oxazaborinin-6-yl)phenyl)amino)ethyl)-6-methyl-4H-chromen-4-one (70 mg, 133 μmol, 1 eq) was separated by SFC (column: DAICEL CHIRALPAK IG (250 mm×30 mm, 10 um); mobile phase:[CO2-IPA (0.1% □H3H2O)]; B %:50%, isocratic elution mode) to give the title compound, Isomer 1 (30.7 mg, 58.22 μmol, 43.61% yield) as a white solid and the title compound, Isomer 2 (30.7 mg, 58.18 μmol, 43.58% yield) as a white solid. Isomer 1: ¹H NMR (DMSO-d₆, 400 MHz) δ 8.77-8.47 (m, 1H), 8.28-7.94 (m, 1H), 7.66-7.47 (m, 4H), 6.91-6.85 (m, 2H), 6.46-6.15 (m, 1H), 6.13-6.09 (m, 1H), 4.96-4.50 (m, 2H), 2.33-2.29 (m, 4H), 1.86-1.75 (m, 2H), 1.75-1.72 (m, 3H), 1.43-1.04 (m, 8H). MS (ESI): mass calcd. For C₃₁H₃₀BFN₂O₄ 524.40, m/z found 525.2 [M+H]⁺. HPLC: 99.44% (220 rn), 98.66% (254 nm). Chiral purity: 100% ee. Isomer 2: ¹H NMR (DMSO-d₆, 400 MHz) δ 7.85-7.64 (m, 2H), 7.64-7.62 (m, 1H), 7.53-7.51 (m, 2H), 7.49-7.40 (m, 1H), 6.89-6.85 (m, 2H), 6.46-6.45 (m, 1H), 6.17-6.09 (m, 1H), 4.99-4.55 (m, 2H), 2.33-2.22 (m, 4H), 1.75-1.75 (m, 2H), 1.72-1.72 (m, 3H), 1.43-1.40 (m, 3H), 1.38-1.15 (m, 5H). MS (ESI): mass calcd. For C₃₁H₃₀BFN₂O₄ 524.40, m/z found 525.2 [M+H]⁺. HPLC: 99.37% (220 nm), 99.55% (254 nm). Chiral purity: 97.52% ee.

### Example 45

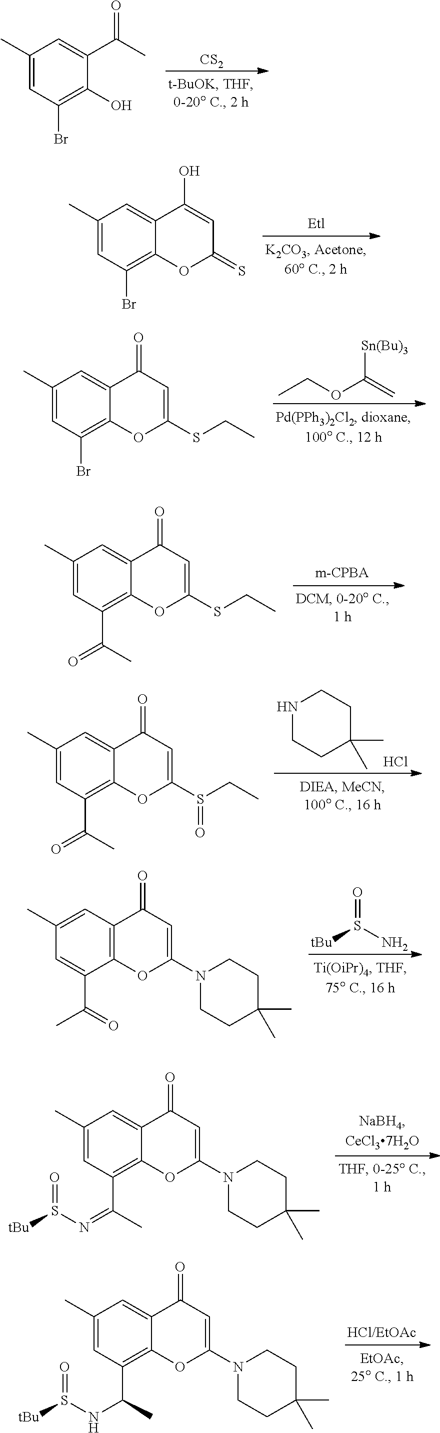

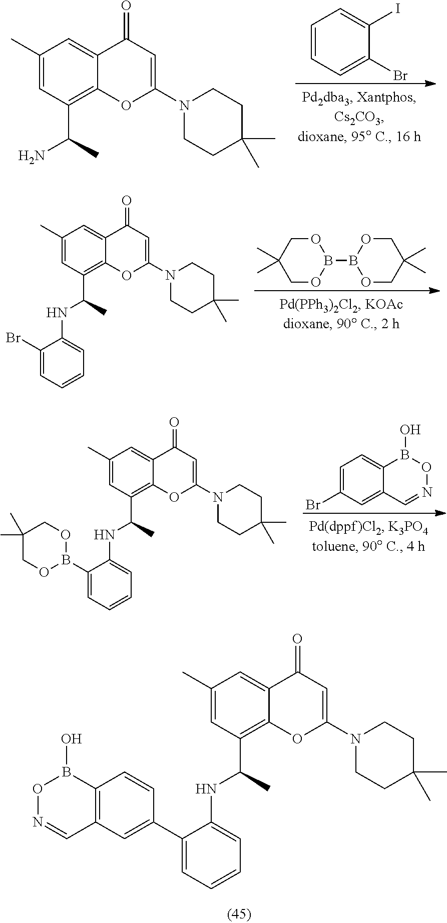

### Preparation of 8-bromo-4-hydroxy-6-methyl-chromene-2-thione

To a solution of 1-(3-bromo-2-hydroxy-5-methyl-phenyl) ethanone (30.00 g, 130.96 mmol, 1 eq) in THF (300 mL) was added t-BuOK (44.09 g, 392.89 mmol, 3 eq) in portions at 0° C., the reaction mixture was stirred at 0° C. for 10 min. Then CS₂ (14.96 g, 196.45 mmol, 11.84 mL, 1.5 eq) was added dropwise to the mixture above at 0° C. and the reaction mixture was stirred at 20° C. for 2 h. The reaction mixture was quenched by aq.sat Na₂SO₃ (400 mL), acidified with 6N HCl to pH=2 and then extracted with EtOAc (150 mL×3). The combined organic layers were washed with brine (300 mL), dried over Na₂SO₄, filtered and concentrated under reduced pressure to give a residue. The crude product was triturated with MTBE (300 mL) at 25° C. for 30 min to give the title compound (10.00 g, 36.88 mmol, 28.16% yield) as a yellow solid. ¹H NMR (DMSO-d₆, 400 MHz) δ 7.87 (d, J=1.6 Hz, 1H), 7.66 (s, 1H), 6.65 (s, 1H), 2.39 (s, 3H).

### Preparation of 8-bromo-2-ethylsulfanyl-6-methyl-chromen-4-one

To a solution of 8-bromo-4-hydroxy-6-methyl-chromene-2-thione (20.00 g, 73.77 mmol, 1 eq) and K2CO₃ (12.23 g, 88.52 mmol, 1.2 eq) in acetone (400 mL) was added EtI (48.32 g, 309.81 mmol, 24.78 mL, 4.2 eq) dropwise at 25° C., the reaction mixture was directly moved to the oil-bath (pre-heated to 60° C.) and stirred at 60° C. for 2 h. The reaction mixture was quenched by sat.aq.NH₄Cl (800 mL) and then extracted with EtOAc (500 mL). The combined organic layers were washed with brine (500 mL×2), dried over Na₂SO₄, filtered and concentrated under reduced pressure to give the title compound (15.00 g, 50.14 mmol, 67.97% yield) as a yellow solid. ¹H NMR (DMSO-d₆, 400 MHz) δ 7.90 (s, 1H), 7.75 (s, 1H), 6.39 (s, 1H), 3.24 (dd, J=7.2 Hz, 14.4 Hz, 2H), 2.40 (s, 3H), 1.39 (t, J=7.2 Hz, 3H).

### Preparation of 8-acetyl-2-ethylsulfanyl-6-methyl-chromen-4-one

To a solution of tributyl(1-ethoxyvinyl)stannane (67.23 g, 186.16 mmol, 62.89 mL, 1.21 eq) and 8-bromo-2-ethylsulfanyl-6-methyl-chromen-4-one (46.00 g, 153.75 mmol, 1 eq) in dioxane (600 mL) was added Pd(PPh₃)₂Cl₂ (10.79 g, 15.38 mmol, 0.1 eq) in portions at 25° C. The reaction mixture was directly moved to the oil-bath (pre-heated to 100° C.) and stirred at 100° C. for 12 h. The reaction mixture was cooled to 20° C., quenched with sat. aq. KF (1000 mL), stirred for 0.5 h and then filtered. The filter cake was washed with EtOAc (200 mL×2). The filtrate was extracted with EtOAc (300 mL×2), washed with brine (600 mL), dried over Na₂SO₄, filtered and concentrated under reduced pressure. The crude product was further triturated with MTBE (300 mL) at 25° C. for 30 min to give the title compound (28.00 g, 106.74 mmol, 69.42% yield) as a yellow solid. ¹H NMR (DMSO-d₆, 400 MHz) δ 7.97 (s, 1H), 7.95 (s, 1H), 6.39 (s, 1H), 3.21 (dd, J=7.2 Hz, 14.4 Hz, 2H), 2.66 (s, 3H), 2.43 (s, 3H) 1.34 (t, J=7.2 Hz, 3H).

### Preparation of 8-acetyl-2-ethylsulfinyl-6-methyl-chromen-4-one

To a solution of 8-acetyl-2-ethylsulfanyl-6-methyl-chromen-4-one (28.00 g, 106.74 mmol, 1 eq) in DCM (350 mL) was added m-CPBA (26.00 g, 128.09 mmol, 1.2 eq) in portions at 0° C. The reaction mixture was warmed to 20° C. and stirred at 20° C. for 1 h. The reaction mixture was quenched by sat.aq.Na₂SO₃ (300 mL), diluted with H₂O (1000 mL) and extracted with DCM (500 mL×3). The combined organic layers were washed with brine (500 mL), dried over Na₂SO₄, filtered and concentrated under reduced pressure. The crude product was triturated with MTBE (50 mL) at 25° C. for 30 min to give the title compound (15.00 g, 53.89 mmol, 50.49% yield) as a yellow solid, which contains some over-oxidation byproduct sulfone and used directly for next step without further purification. ¹H NMR (DMSO-d₆, 400 MHz) δ 8.08-8.04 (m, 2H), 6.98 (s, 1H), 6.71 (s, 1H), 3.63 (dd, J=7.2 Hz, 14.4 Hz, 2H), 3.46-3.34 (m, 1H), 3.29 (s, 1H), 3.15-3.06 (m, 1H), 2.74 (s, 1H), 2.68 (s, 2H), 2.47 (s, 2H), 1.29-1.15 (m, 3H).

### Preparation of 8-acetyl-2-(4,4-dimethyl-1-piperidyl)-6-methyl-chromen-4-one

To a solution of 8-acetyl-2-ethylsulfinyl-6-methyl-chromen-4-one (3.00 g, 10.78 mmol, 1 eq) and 4,4-dimethylpiperidine (2.74 g, 18.32 mmol, 1.7 eq, HCl) in MeCN (30 mL) was added DIEA (4.18 g, 32.34 mmol, 5.63 mL, 3 eq) dropwise at 25° C., the reaction mixture was directly moved to the oil-bath (pre-heated to 100° C.) and stirred at 100° C. for 16 h. The reaction mixture was quenched by adding water (60 mL) at 25° C., and then extracted with EtOAc (20 mL×3). The combined organic layers were washed with brine (25 mL×3), dried over Na₂SO₄, filtered and concentrated under reduced pressure to give a residue. The residue was purified by flash silica gel chromatography (Biotage®; 80 SepaFlash® Silica Flash Column, Eluent of 0~80% Ethyl acetate/Petroleum ether gradient@150 L/mmin) to give the title compound (3.50 g, crude) as a gray solid.

### Preparation of (NZ, R)—N-[1-[2-(4,4-dimethyl-1-piperidyl)-6-methyl-4-oxo-chromen-8-yl]ethylidene]-2-methyl-propane-2-sulfinamide

To a solution of (R)-2-methylpropane-2-sulfinamide (1.93 g, 15.95 mmol, 2 eq) and 8-acetyl-2-(4,4-dimethyl-1-piperidyl)-6-methyl-chromen-4-one (2.50 g, 7.98 mmol, 1 eq) in THF (30 mL) was added tetraisopropoxytitanium (9.07 g, 31.91 mmol, 9.42 mL, 4 eq) dropwise at 25° C., the reaction mixture was directly moved to the oil-bath (pre-heated to 75° C.) and stirred at 75° C. for 16 h. The reaction mixture was quenched by adding water (60 mL) at 25° C., and then extracted with EtOAc (50 mL×3). The combined organic layers were washed with brine (100 mL), dried over Na₂SO₄, filtered and concentrated under reduced pressure to give the title compound (2.00 g, 4.80 mmol, 60.18% yield) as a yellow solid. ¹H NMR (DMSO-d₆, 400 MHz) δ 7.80-7.70 (m, 1H), 7.52-7.36 (m, 1H), 5.53-5.49 (m, 1H), 5.50-5.45 (m, 4H), 2.42-2.38 (m, 3H), 1.39-1.37 (m, 4H), 1.19 (m, 3H), 1.07 (s, 9H), 0.98-0.96 (m, 6H).

### Preparation of (R)—N-[(1R)-1-[2-(4,4-dimethyl-1-piperidyl)-6-methyl-4-oxo-chromen-8-yl]ethyl]-2-methyl-propane-2-sulfinamide

To a solution of (NZ,R)—N-[1-[2-(4,4-dimethyl-1-piperidyl)-6-methyl-4-oxo-chromen-8-yl]ethylidene]-2-methyl-propane-2-sulfinamide (4.00 g, 9.60 mmol, 1 eq) in THF (60 mL) was added CeCl₃·7H₂₀ (1.79 g, 4.80 mmol, 456.32 μL, 0.5 eq) and NaBH₄ (770 mg, 20.35 mmol, 2.12 eq) in portions at 0° C., the reaction mixture was stirred at 20° C. for 2 h. The reaction mixture was poured into ice water (100 mL) at 0° C. and then extracted with DCM (45 mL×3). The combined organic phase was washed with brine (100 mL), dried over Na₂SO₄, filtered and concentrated under reduced pressure. The residue was purified by prep-HPLC (column: Welch Xtimate C18 250×70 mm×10 um; mobile phase: [H₂O (10 mM NH₄HCO₃)-AC□]; gradient:33%-63% B over 17.0 min) to give the title compound (1.90 g, 4.54 mmol, 47.27% yield) as a white solid.

### Preparation of 8-[(1R)-1-aminoethyl]-2-(4,4-dimethyl-1-piperidyl)-6-methyl-chromen-4-one

To a solution of (R)—N-[(1R)-1-[2-(4,4-dimethyl-1-piperidyl)-6-methyl-4-oxo-chromen-8-yl]ethyl]-2-methyl-propane-2-sulfinamide (1.80 g, 4.30 mmol, 1 eq) in EtOAc (10 mL) was added HCl/EtOAc (4 M, 3.23 mL, 3 eq) dropwise at 25° C., the reaction mixture was stirred at 25° C. for 1 h. The reaction suspension was filtered. The filter cake was dissolved in H₂O (30 mL), adjusted pH=10 with concentrated NH₃·H₂O at 25° C. and then extracted with EtOAc (15 mL×3). The combined organic layers were washed with brine (30 mL), dried over Na₂SO₄, filtered and concentrated under reduced pressure to give the title compound (1.10 g, 3.50 mmol, 81.36% yield) as a white solid. ¹H NMR (DMSO-d₆, 400 MHz) δ 7.61 (s, 1H), 7.54 (s, 1H), 5.46 (s, 1H), 4.46 (q, J=4.0 Hz, 8 Hz, 1H), 3.51 (t, J=8.0 Hz, 4H), 2.36 (s, 3H), 1.98 (s, 2H), 1.42 (t, J=4.0 Hz, 4H), 1.29 (d, J=8.0 Hz, 3H), 0.98 (s, 6H).

### Preparation of 8-[(1R)-1-(2-bromoanilino)ethyl]-2-(4,4-dimethyl-1-piperidyl)-6-methyl-chromen-4-one-one

The title compound was prepared as a yellow oil (1.00 g, 2.13 mmol, 83.73% yield) according to the similar procedure in Example 78, using 8-[(1R)-1-aminoethyl]-2-(4,4-dimethyl-1-piperidyl)-6-methyl-chromen-4-one (800 mg, 2.54 mmol) and 1-bromo-2-iodo-benzene (1.44 g, 5.09 mmol, 653.48 μL, 2 eq).

¹H NMR (DMSO-d₆, 400 MHz) δ 7.58 (s, 1H), 7.43-7.42 (m, 2H), 7.05 (t, J=0.0 Hz, 1H), 6.52 (t, J=8.0 Hz, 1H), 6.41 (d, J=1.2 Hz, 1H), 5.51 (s, 1H), 5.29 (d, J=8.0 Hz, 1H), 5.09-4.99 (m, 1H), 3.54 (t, J=4.0 Hz, 4H), 2.29 (s, 3H), 1.59 (s, 3H), 1.42 (t, J=4.0 Hz, 4H), 0.97 (s, 6H).

### Preparation of 8-[(1R)-1-[2-(5,5-dimethyl-1,3,2-dioxaborinan-2-yl)anilino]ethyl]-2-(4,4-dimethyl-1-piperidyl)-6-methyl-chromen-4-one

The title compound was prepared as a black solid (1.20 g, crude) according to the procedures described herein and the synthetic scheme, using 8-[(1R)-1-(2-bromoanilino)ethyl]-2-(4,4-dimethyl-1-piperidyl)-6-methyl-chromen-4-one (1.00 g, 2.13 mmol) and B₂neop₂ (2.41 g, 10.65 mmol, 5 eq). The product was used directly for next step without further purification.

### Preparation of 2-(4,4-dimethyl-1-piperidyl)-8-[(1R)-1-[2-(1-hydroxy-2,3,1-benzoxaz aborinin-6-yl)anilino]ethyl]-6-methyl-chromen-4-one

The title compound was prepared as a white solid (420 mg, 78.44 μmol, 3.94% yield) according to the procedures described herein and the synthetic scheme, using 8-[(1R)-1-[2-(5,5-dimethyl-1,3,2-dioxaborinan-2-yl)anilino]ethyl]-2-(4,4-dimethyl-1-piperidyl)-6-methyl-chromen-4-one (1.00 g, 1.99 mmol, 1 eq) and 6-bromo-1-hydroxy-2,3,1-benzoxazaborinine (584 mg, 2.59 mmol, 1.3 eq). ¹H NMR (DMSO-d₆, 400 MHz) δ 9.43 (s, 1H), 8.65 (s, 1H), 8.13 (d, J=8.0 Hz, 1H), 7.82-7.79 (m, 2H), 7.55 (s, 1H), 7.43 (d, J=1.6 Hz, 1H), 7.11 (t, J=8.0 Hz, 1H), 7.04 (d, J=8.0 Hz, 1H), 6.72 (t, J=8.0 Hz, 1H), 6.56 (d, J=8.0 Hz, 1H), 5.46 (s, 1H), 5.08 (d, J=8.0 Hz, 1H), 4.93-4.84 (m, 1H), 3.40-3.36 (m, 3H), 2.30 (s, 3H), 1.42 (d, J=8.0 Hz, 3H), 1.38-1.31 (m, 4H), 0.95 (s, 6H). MS (ESI): mass calcd. For C₃₂H₃₄BN₃O₄ 535.26, m/z found 536.3 [M+H]⁺. HPLC: 97.71% (220 nm), 96.40% (254 nm). Chiral purity: 98.78% ee.

### Example 46

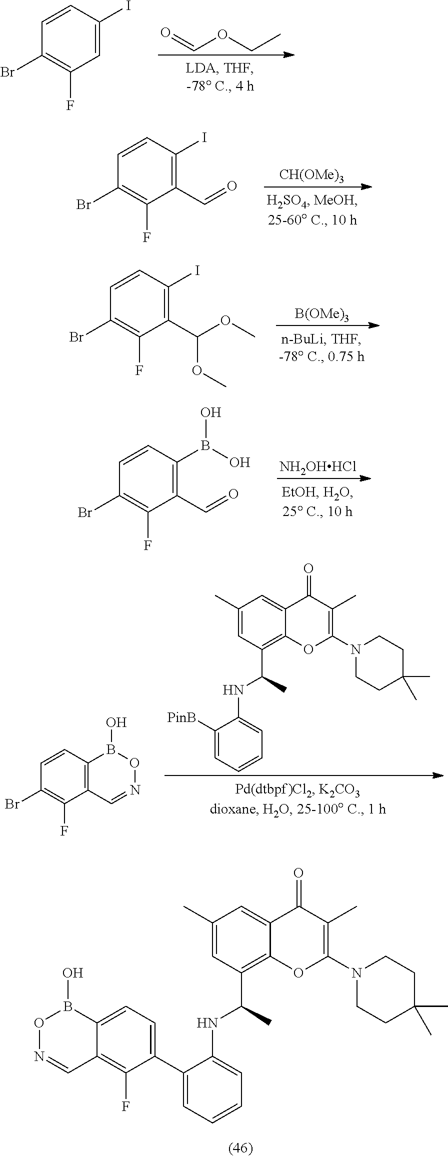

### Preparation of 3-bromo-2-fluoro-6-iodobenzaldehyde

To a stirred solution of 1-bromo-2-fluoro-4-iodo-benzene (3.00 g, 9.97 mmol, 1 eq) in THF (30 mL) at −78° C. was added LDA (2 M, 7.48 mL, 1.5 eq) dropwise and the resulting mixture was stirred at −78° C. for 2 h. Then a solution of ethyl formate (2.22 g, 29.91 mmol, 2.41 mL, 3 eq) was added dropwise and stirred at −78° C. for 2 h. The reaction mixture was warmed up to 0° C. and quenched by sat. aq. NH₄Cl (20 mL) and then directly extracted with EtOAc (10 mL×3). The combined organic layers were washed with brine (5 mL×2), dried over Na₂SO₄, filtered and concentrated under reduced pressure to give a residue. The residue was purified by flash silica gel chromatography (Biotage®; 20 g SepaFlash® Silica Flash Column, Eluent of 0~10% Ethyl acetate/Petroleum ether gradient@60 mL/min). The organic phase was concentrated under reduced pressure to give the title compound (2.77 g, 8.42 mmol, 84.47% yield) as a yellow solid. ¹H NMR (DMSO-d₆, 400 MHz) δ 9.93 (s, 1H), 7.83 (d, J=8.4 Hz, 1H), 7.70 (t, J=7.2 Hz, 1H).

### Preparation of 1-bromo-3-(dimethoxymethyl)-2-fluoro-4-iodobenzene

To a solution of 3-bromo-2-fluoro-6-iodo-benzaldehyde (2.77 g, 8.42 mmol, 1 eq) and trimethoxymethane (1.79 g, 16.84 mmol, 1.85 mL, 2 eq) in MeOH (30 mL) was added H₂SO₄ (84 mg, 842 μmol, 46 μL, 0.1 eq) at 25° C. The mixture was stirred at 60° C. for 10 h. The reaction mixture was cooled to 0° C., quenched with sat. aq. NaHCO₃ (30 mL) and extracted with EtOAc (50 mL×3). The combined organic layers were washed with brine (20 mL×2), dried over Na₂SO₄, filtered and concentrated under reduced pressure to give the title compound (3.10 g, crude) as a brown solid. ¹H NMR (DMSO-d₆, 400 MHz) δ 7.67 (d, J=8.4 Hz, 1H), 7.46 (t, J=7.6 Hz, 1H), 5.48 (s, 1H), 3.37 (s, 6H).

### Preparation of (4-bromo-3-fluoro-2-formylphenyl)boronic acid

To a stirred solution of 1-bromo-3-(dimethoxymethyl)-2-fluoro-4-iodo-benzene (3.00 g, 8.00 mmol, 1 eq) in THF (30 mL) at −78° C. was added n-BuLi (2.5 M, 3.20 mL, 1 eq) dropwise and the resulting mixture was stirred at −78° C. for 15 min. Then a solution of trimethyl borate (831 mg, 8.00 mmol, 904 μL, 1 eq) was added dropwise and stirred at −78° C. for 30 min. The mixture was warmed to 0° C., quenched with HCl (2 mL, 3 N) slowly and extracted with EtOAc (30 mL×3). The combined organic phase was washed with brine (20 mL), dried over Na₂SO₄, filtered and concentrated in vacuo.

The residue was purified by flash silica gel chromatography (Biotage®; 20 g SepaFlash® Silica Flash Column, Eluent of 0~50% Ethyl acetate/Petroleum ether gradient@60 mL/min). The organic phase was concentrated under reduced pressure to give the title compound (1.10 g, 4.46 mmol, 55.70% yield) as brown oil. ¹H NMR (DMSO-d₆, 400 MHz) δ 10.24 (s, 1H), 8.51 (s, 2H), 7.68 (d, J=6.0 Hz, 1H), 7.59 (d, J=8.0 Hz, 1H).

### Preparation of 6-bromo-5-fluoro-1H-benzo[d][1,2,6]oxazaborinin-1-ol

To a solution of (4-bromo-3-fluoro-2-formyl-phenyl)boronic acid (1.00 g, 4.05 mmol, 1 eq) in EtOH (10 mL) and H₂O (1 mL) was added hydroxylamine;hydrochloride (563 mg, 8.10 mmol, 2 eq) in one portion at 25° C., the mixture was stirred at 25° C. for 10 h. The reaction mixture was concentrated in vacuo and the residue was purified by prep-HPLC (column: Waters Xbridge Prep OBD C18 150×40 mm×10 um; mobile phase: [H₂O (10 mM NH₄HCO₃)-AC□]; gradient: 10%-40% B over 8.0 min) to give the title compound (70 mg, 287 μmol, 7.09% yield) was obtained as a white solid. ¹H NMR (DMSO-d₆, 400 MHz) δ 9.25 (s, 1H), 8.76 (s, 1H), 8.05 (t, J=7.6 Hz, 1H), 7.77 (d, J=8.0 Hz, 1H).

### Preparation of 2-(4,4-dimethyl-1-piperidyl)-8-[(1R)-1-[2-(5-fluoro-1-hydroxy-2,3,1-benzoxazaborinin-6-yl)anilino]ethyl]-3,6-dimethyl-chromen-4-one

A mixture of 6-bromo-5-fluoro-1-hydroxy-2,3,1-benzoxazaborinine (40 mg, 164.05 μmol, 1 eq), 2-(4,4-dimethyl-1-piperidyl)-3,6-dimethyl-8-[(1R)-1-[2-(4,4,5,5-tetramethyl-1,3,2-dioxabo rolan-2-yl)anilino]ethyl]chromen-4-one (131 mg, 246 μmol, 1.5 eq), ditert-butyl(cyclopentyl) phosphane;dichloropalladium;iron (11 mg, 16 μmol, 0.1 eq), K₂CO₃ (45 mg, 328 μmol, 2 eq) in dioxane (1 mL) and H₂O (0.2 mL) at 25° C. was degassed and purged with N₂ for 3 times, and then the mixture was heated to 100° C. and stirred at 100° C. for 1 h under N₂ atmosphere. The reaction mixture was cooled to 25° C. and filtered. The filtrate was directly purified by prep-HPLC (column: Phenomenex Luna C18 75×30 mm×3 um; mobile phase: [H₂O (0.2% FA)-AC□]; gradient:45%-75% B over 8.0 min). The residue was purified by prep-HPLC (column: Waters Xbridge BEH C18 100×30 mm×10 um; mobile phase: [H₂O (10 nM NH₄HCO₃)-AC□]; gradient:35%-75% B over 8.0 min) to give the title compound (1.3 mg, 2.29 μmol, 1.40% yield) as a white solid. ¹H NMR (DMSO-d₆, 400 MHz) δ 8.76 (s, 1H), 7.92 (d, J=8.0 Hz, 1H), 7.74 (d, J=7.2 Hz, 1H), 7.59 (s, 1H), 7.44 (s, 1H), 7.10 (d, J=8.0 Hz, 1H), 7.01 (d, J=7.2 Hz, 1H), 6.67 (t, J=7.6 Hz, 1H), 6.52 (d, J=8.4 Hz, 1H), 5.05 (d, J=6.8 Hz, 1H), 4.96 (t, J=6.4 Hz, 1H), 3.44-3.42 (m, 4H), 2.31 (s, 3H), 1.91 (s, 3H), 1.43 (d, J=6.0 Hz, 7H), 0.98 (s, 6H). MS (ESI): mass calcd. For C₃₃H₃₅BFN₃O₄ 567.27, m/z found 568.3 [M+H]⁺. HPLC: 97.43% (220 nm), 97.29% (254 nm). Chiral purity: 100 ee %.

### Example 47

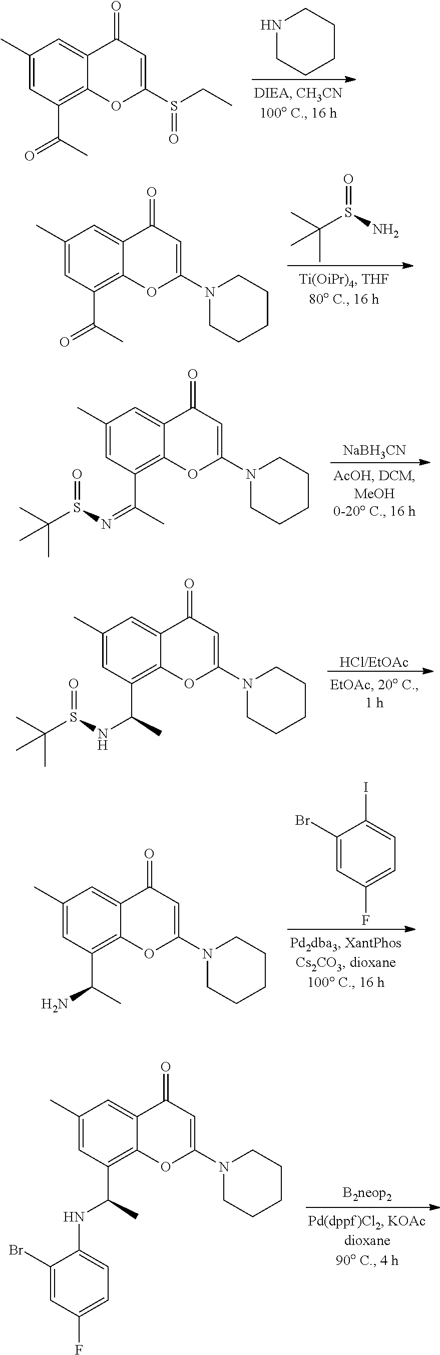

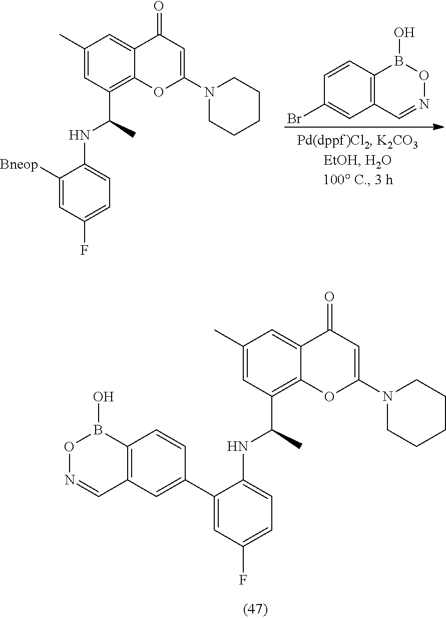

### Preparation of 8-acetyl-6-methyl-2-(1-piperidyl)chromen-4-one

To a solution of 8-acetyl-2-ethylsulfinyl-6-methyl-chromen-4-one (5.00 g, 17.9 mmol, 1 eq) and piperidine (2.29 g, 26.9 mmol, 2.66 mL, 1.5 eq) in CH₃CN (50 mL) was added DIEA (6.97 g, 53.9 mmol, 9.39 mL, 3 eq) in one portion at 20° C., the reaction mixture was stirred at 100° C. for 16 h. The reaction mixture was cooled to 20° C. and directly concentrated under reduced pressure. The residue was poured into ice-water (w/w=1/1) (150 mL) and extracted with EtOAc (100 mL×3). The combined organics were washed with brine (50 mL), dried with anhydrous Na₂SO₄, filtered and concentrated in vacuo. The residue was purified by flash silica gel chromatography (ISCO®; 40 g SepaFlash® Silica Flash Column, Eluent of 0~100% Ethyl acetate/Petroleum ether gradient@100 mL/min) to give the title compound (3.70 g, 12.97 mmol, 72.18% yield) as a yellow solid. ¹H NMR (DMSO-d6, 400 MHz) δ 8.16 (d, J=2.4 Hz, 1H), 7.74 (d, J=1.6 Hz, 1H), 5.62 (s. 1H), 3.61-3.60 (m, 4H), 2.68 (s, 3H), 2.46 (s, 3H), 1.72 (s, 6H).

### Preparation of (NZ,R)-2-methyl-N-[1-[6-methyl-4-oxo-2-(1-piperidyl)chromen-8-yl]ethylidene]propane-2-sulfinamide

To a mixture of 8-acetyl-6-methyl-2-(1-piperidyl)chromen-4-one (2.70 g, 9.46 mmol, 1 eq) and (R)-2-methylpropane-2-sulfinamide (2.29 g, 18.9 mmol, 2 eq) in THF (100 mL) was added Ti(i-PrO)₄ (10.8 g, 37.8 mmol, 11.2 mL, 4 eq) at 20° C. dropwise, the reaction mixture was stirred at 80° C. for 16 h. The reaction mixture was quenched by adding water (200 mL) at 20° C., filtered and the filtrate was extracted with EtOAc (100 mL×3). The combined organic layers were washed with brine (50 mL), dried over Na₂SO₄, filtered and concentrated under reduced pressure to give a residue. The residue was purified by flash silica gel chromatography (ISCO®; 40 g SepaFlash® Silica Flash Column, Eluent of 0~30% Ethyl acetate/Petroleum ether gradient@100 mL/min) to give the title compound (4.00 g, crude) as a yellow solid (a mixture of Z/E isomers), which was used directly for next step without further purification.

### Preparation of 2-methyl-N-[(1R)-1-[6-methyl-4-oxo-2-(1-piperidyl)chromen-8-yl]ethyl]propane-2-sulfinamide

To s solution of (NZ,R)-2-methyl-N-[1-[6-methyl-4-oxo-2-(1-piperidyl)chromen-8-yl]ethylidene]propane-2-sulfinamide (5.00 g, 12.8 mmol, 1 eq) in MeOH (30 mL)/DCM (30 mL)/AcOH (6.18 g, 103 mmol, 5.89 mL, 7.99 eq) was added NaBH₃CN (2.43 g, 38.6 mmol, 3.00 eq) at 0° C. in portions, the reaction mixture was stirred at 20° C. for 16 h. The residue was poured into ice-water (w/w=1/1) (100 mL) and extracted with DCM (50 mL×3). The combined organic phase was washed with brine (30 mL), dried with anhydrous Na₂SO₄, filtered and concentrated in vacuo. The residue was purified by prep-HPLC (column: Welch Xtimate C18 250×100 mm×10 um; mobile phase: [H₂O (10 mM NH₄HCO₃)-AC□]; gradient: 25%-55% B over 18.0 min) to give the title compound (3.00 g, 7.68 mmol, 59.69% yield) as a yellow solid. ¹H NMR (DMSO-d₆, 400 MHz) δ 7.91 (s, 1H), 7.41 (d, J=2.0 Hz, 1H), 5.62 (s, 1H), 5.09-5.04 (m, 1H), 3.55-3.54 (m, 4H), 3.46 (d, J=2.8 Hz, 1H), 2.43 (s, 3H), 1.72-1.71 (m, 6H), 1.60 (d, J=6.8 Hz, 3H), 1.25 (s, 9H).

### Preparation of 8-[(1R)-1-aminoethyl]-6-methyl-2-(1-piperidyl)chromen-4-one

To a solution of 2-methyl-N-[(1R)-1-[6-methyl-4-oxo-2-(1-piperidyl)chromen-8-yl]ethyl]propane-2-sulfinamide (3.00 g, 7.68 mmol, 1 eq) in EtOAc (5 mL) was added HCl/EtOAc (5 mL) at 20° C. dropwise, the reaction mixture was stirred at 20° C. for 1 h. The resulting reaction suspension was filtered and the filter cake was dissolved in H₂O (50 mL), adjusted pH=12 with concentrated NH₃·H₂O at 0° C. and extracted with EtOAc (30 mL×3). The combined organic phase was washed with brine (30 mL), dried over Na₂SO₄, filtered and concentrated under reduced pressure to give the title compound (2.00 g, 6.98 mmol, 90.92% yield) as a yellow solid, which was used directly for next step without further purification. ¹H NMR (DMSO-d₆, 400 MHz) δ 7.82 (d, J=1.6 Hz, 1H), 7.41 (d, J=2.0 Hz, 1H), 5.51 (s. 1H), 4.64 (q, J=6.8 Hz, 1H), 3.52-3.51 (m, 4H), 2.41 (s, 3H), 1.72-1.71 (m, 6H), 1.50 (d, J=6.8 Hz, 1H).

### Preparation of (R)-8-(1-((2-bromo-4-fluorophenyl)amino)ethyl)-6-methyl-2-(piperidin-1-yl)-4H-chromen-4-one

The title compound was prepared (800 mg, crude) according to the similar procedure in Example 78, using 8-[(1R)-1-aminoethyl]-6-methyl-2-(1-piperidyl)chromen-4-one (600 mg, 2.10 mmol, 1 eq) and 2-bromo-4-fluoro-1-iodo-benzene (1.26 g, 4.19 mmol, 2 eq). ¹H NMR (DMSO, 400 MHz) δ 7.78 (d, J=1.6 Hz, 1H), 7.27 (d, J=2.0 Hz, 1H), 7.16 (dd, J=2.8, 8.0 Hz, 1H), 6.72-6.67 (m, 1H), 6.14 (dd, J=4.8, 9.2 Hz, 1H), 5.69 (s, 1H), 4.83-4.79 (m, 1H), 4.46 (d, J=2.8 Hz, 1H), 3.50-3.47 (m, 4H), 2.29 (s, 3H), 1.65 (s, 6H), 1.58 (d, J=6.8 Hz, 3H).

### Preparation of 8-[(1R)-1-[2-(5,5-dimethyl-1,3,2-dioxaborinan-2-yl)-4-fluoro-anilino]ethyl]-6-methyl-2-(1-piperidyl)chromen-4-one

The title compound was prepared as a yellow solid (600 mg, crude) according to the procedures described herein and the synthetic scheme using 8-[(1R)-1-(2-bromo-4-fluoro-anilino)ethyl]-6-methyl-2-(1-piperidyl) chromen-4-one (800 mg, 1.74 mmol, 1 eq) and B₂neop₂ (1.97 g, 8.71 mmol, 5 eq). The product was used directly for next step without further purification.

### Preparation of (R)-8-(1-((4-fluoro-2-(1-hydroxy-1H-benzo[d][1,2,6]oxazaborinin-6-yl)phenyl)amino)ethyl)-6-methyl-2-(piperidin-1-yl)-4H-chromen-4-one

The title compound was prepared as an off white solid (22 mg, 40.26 μmol, 3.96% yield) according to the procedures described herein and the synthetic scheme, using 8-[(1R)-1-[2-(5,5-dimethyl-1,3,2-dioxaborinan-2-yl)-4-fluoro-anilino]ethyl]-6-methyl-2-(1-piperidyl)chromen-4-one (500 mg, 1.02 mmol, 1 eq) and 6-bromo-1-hydroxy-2,3,1-benzoxazaborinine (344 mg, 1.52 mmol, 1.5 eq). ¹H NMR (DMSO-d₆, 400 MHz) δ 9.43 (s, 1H), 8.64 (s, 1H), 8.13 (d, J=8.0 Hz, 1H), 7.85-7.82 (m, 2H), 7.55 (s, 1H), 7.40 (s, 1H), 6.97-6.92 (m, 2H), 6.54-6.52 (m, 1H), 5.45 (s. 1H), 5.05 (d, J=7.2 Hz, 1H), 4.83-4.78 (m, 1H), 3.40-3.38 (m, 4H), 2.30 (s, 3H), 1.59-1.55 (m, 6H), 1.39 (d, J=6.4 Hz, 3H). MS (ESI): mass calcd. For C₃₀H₂₉BFN₃O₄ 525.22, m/z found 526.3 [M+H]⁺. HPLC: 95.96% (220 nm), 97.91% (254 nm). Chiral purity: 99.22% ee.

### Example 48

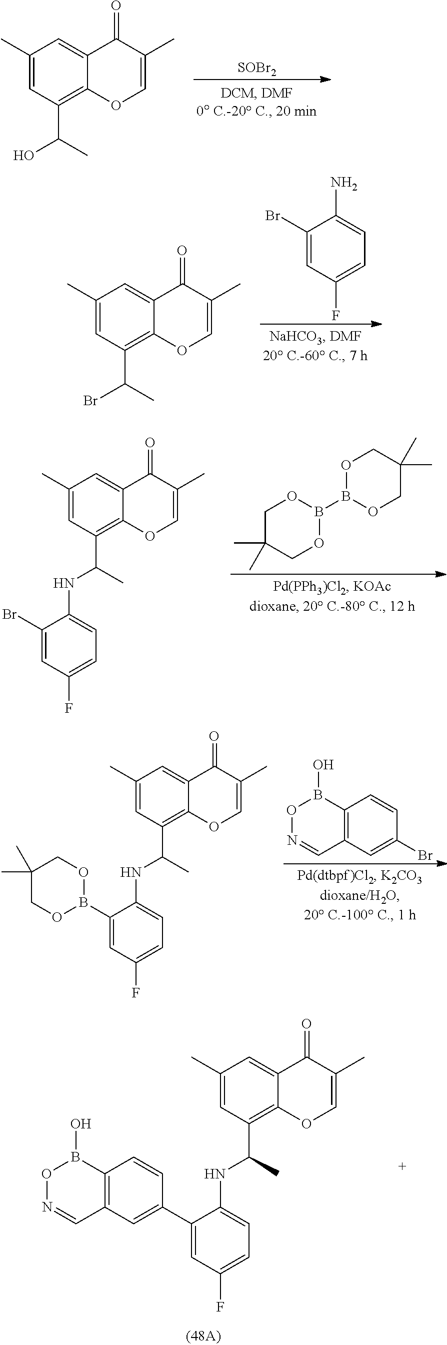

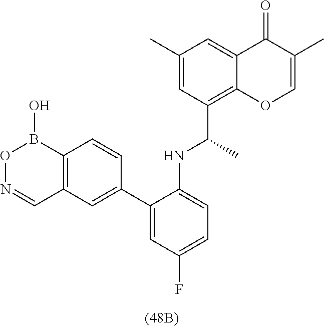 [SMILES: Cc1cc([C@H](C)Nc2ccc(F)cc2-c2ccc3c(c2)C=NOB3O)c2occ(C)c(=O)c2c1]

### Preparation of 8-(1-bromoethyl)-3,6-dimethyl-chromen-4-one

To a solution of 8-(1-hydroxyethyl)-3,6-dimethyl-chromen-4-one (1.0 g, 3.67 mmol, 1 eq) in DCM (10 mL) was added dropwise DMF (2.68 mg, 36.7 μmol, 2.82 μL, 0.01 eq) at 0° C., then to this added dropwise ThionylBromide (991 mg, 4.77 mmol, 369.2 μL, 1.3 eq) at 0° C., the reaction mixture was warmed to 20° C. and stirred at 20° C. for 20 min. The reaction mixture was poured into ice H₂O (10 mL) at 0° C., the aqueous phase extracted with DCM (10 mL×3). The combined organic layers were washed with sat.aq NaHCO₃ until pH is about 8, then the combined organic layers were washed with brine (30 mL), dried over Na₂SO₄, filtered and concentrated under reduced pressure to give a residue. The residue was purified by flash silica gel chromatography (Biotage®; 12 g SepaFlash® Silica Flash Column, Eluent of 0~11% Ethyl acetate/Petroleum ether gradient@40 m/min) to give the title compound (700 mg, 2.49 mmol, 67.92% yield) as a yellow solid. ¹H NMR (CDCl₃, 400 MHz) δ 8.00 (s, 1H), 7.86 (s, 1H), 7.66 (d, J=2.0 Hz, 1H), 5.75 (q, J=6.8 Hz, 1H), 2.47 (s, 3H), 2.12 (d, J=6.8 Hz, 3H), 2.05 (s, 3H).

### Preparation of 8-[1-(2-bromo-4-fluoro-anilino)ethyl]-3,6-dimethyl-chromen-4-one

To a mixture of 8-(1-bromoethyl)-3,6-dimethyl-chromen-4-one (400 mg, 1.42 mmol, 1 eq) and 2-bromo-4-fluoro-aniline (541 mg, 2.85 mmol, 2 eq) in DMF (6 ml) was added NaHCO₃ (239 mg, 2.85 mmol, 111 μL, 2 eq) in one portion at 20° C., then the reaction mixture was directly stirred at 60° C. for 7 h. After the reaction mixture was cooled to 25° C., the reaction mixture was poured into H₂O (50 mL) and then extracted with MTBE (20 mL×3). The combined organic phase was washed with brine (70 mL), dried over Na₂SO₄, filtered and concentrated under reduced pressure to give a residue. The residue was purified by flash silica gel chromatography (Biotage®; 12 g SepaFlash® Silica Flash Column, Eluent of 0~10% Ethyl acetate/Petroleum ether gradient@40 m/min) to give the title compound (320 mg, 820.00 mol, 57.63% yield) as a yellow solid. ¹H NMR (CDCl₃, 400 MHz) δ 7.92 (s, 1H), 7.86 (s, 1H), 7.44 (d, J=2.0 Hz, 1H), 7.22 (dd, J=3.2, 8.4 Hz, 1H), 6.79-6.70 (m, 1H), 6.28-6.22 (m 1H), 5.02 (q, J=6.8 Hz, 1H), 4.92-4.47 (m, 1H), 2.39 (s, 3H), 2.07 (s, 3H), 1.63 (d, J=6.4 Hz, 3H).

### Preparation of 8-(1-((2-(5,5-dimethyl-1,3,2-dioxaborinan-2-yl)-4-fluorophenyl)amino)ethyl)-3,6-dimethyl-4H-chromen-4-one

To a mixture of 8-[1-(2-bromo-4-fluoro-anilino)ethyl]-3,6-dimethyl-chromen-4-one (100 mg, 256 mol, 1 eq) and 2-(5,5-dimethyl-1,3,2-dioxaborinan-2-yl)-5,5-dimethyl-1,3,2-dioxaborinane (174 mg, 769 μmol, 3 eq) in dioxane (2 mL) were added Pd(PPh₃)₂C₁₂ (18.0 mg, 25.6 μmol, 0.1 eq) and KOAc (62.8 mg, 641 μmol, 2.5 eq) in turns at 20° C. under N2, the reaction mixture was bubble N2 for 1 min and then the reaction mixture was directly stirred at 80° C. for 12 h under N₂. 2 parallel reactions were combined for work up. The reaction mixture was cooled to 25° C., filtered and the filtrate was concentrated under reduced pressure to give a residue. The residue was purified by flash silica gel chromatography (Biotage®; 12 g SepaFlash® Silica Flash Column, Eluent of 0~30% Ethyl acetate/Petroleum ether gradient@50 mL/min) to give the title compound (160 mg, 378.00 mol, 73.76% yield) as a white solid. ¹H NMR (CDCl₃, 400 MHz) δ 7.93 (s, 1H), 7.82 (s, 1H), 7.57 (br s, 1H), 7.39 (dd, J=3.2, 9.6 Hz, 1H), 6.88-6.79 (m, 1H), 6.55-6.24 (m, 1H), 5.08-4.94 (m, 1H), 3.82 (s, 4H), 2.40 (s, 3H), 2.05 (s, 3H), 1.62 (d, J=4.0 Hz, 3H), 1.07 (s, 6H).

### Preparation of 8-[(1R)-1-[4-fluoro-2-(1-hydroxy-2,3,1-benzoxazaborinin-6-yl)anilino]ethyl]-3,6-dimethyl-chromen-4-one and 8-[(S)-1-[4-fluoro-2-(1-hydroxy-2,3,1-benzoxazaborinin-6-yl)anilino]ethyl]-3,6-dimethyl-chromen-4-one

To a mixture of 8-[1-[2-(5,5-dimethyl-1,3,2-dioxaborinan-2-yl)-4-fluoro-anilino]ethyl]-3,6-dimethyl-chromen-4-one (110 mg, 260 μmol, 1 eq) and 6-bromo-1-hydroxy-2,3,1-benzoxazaborinine (70.4 mg, 312 μmol, 1.2 eq) in dioxane (1.2 mL)/H₂O (0.2 mL) were added K₂CO₃ (71.8 mg, 5120 mol, 2 eq) and ditert-butyl(cyclopentyl) phosphane;dichloropalladium;iron (17.0 mg, 26.0 μmol, 0.1 eq) in turns at 20° C. under N₂, the reaction mixture was bubble N₂ for 1 min and then the reaction mixture was directly stirred at 100° C. for 1 h under N₂. The reaction mixture was cooled to 25° C., filtered and the filtrate concentrated under reduced pressure to give a residue. The residue was purified by prep-HPLC (column: Phenomenex Luna C18 80×30 mm×3 um; mobile phase: [H₂O (0.1% TFA)-AC□]; gradient: 40%-75% B over 8.0 min) to give 8-[1-[4-fluoro-2-(1-hydroxy-2,3,1-benzoxazaborinin-6-yl)anilino]ethyl]-3,6-dimethyl-chromen-4-one (40 mg, 87.67 μmol, 33.73% yield) as a yellow solid. The racemate product (40 mg) was further separated by SFC (column: DAICEL CHIRALCEL OD (250 mm×30 mm, 10 um); mobile phase: [CO₂—IPA]; B %:45%, isocratic elution mode) to give the title compound, Isomer 1 (11.2 mg, 24.26 μmol, 9.34% yield) as a white solid and the title compound, Isomer 2 (12.6 mg, 27.38 μmol, 10.54% yield) as a white solid. Isomer 1: ¹H NMR (DMSO-d₆, 400 MHz) δ 9.45 (s, 1H), 8.66 (s, 1H), 8.15 (d, J=7.6 Hz, 1H), 8.12 (s, 1H), 7.84 (d, J=8.0 Hz, 1H), 7.82 (s, 1H), 7.70 (s, 1H), 7.54 (s, 1H), 6.97-6.85 (m 2H), 6.55-6.40 (m, 1H), 5.05 (d, J=7.6 Hz, 1H), 4.92-4.81 (m 1H), 2.35 (s, 3H), 1.91 (s, 3H), 1.39 (d, J=6.4 Hz, 3H). MS (ESI): mass calcd. For C₂₆H₂₂BFN₂O₄ 456.28, m/z found 457.2 [M+H]⁺. HPLC: 98.85 (220 nm), 99.31 (254 nm). Chiral purity: 100% ee. Isomer 2: ¹H NMR (DMSO-d₆, 400 MHz) δ 9.45 (s, 1H), 8.66 (s, 1H), 8.15 (d, J=7.6 Hz, 1H), 8.12 (s, 1H), 7.85 (d, J=7.6 Hz, 1H), 7.83 (s, 1H), 7.70 (s, 1H), 7.54 (d, J=2.0 Hz, 1H), 6.99-6.90 (m 2H), 6.50 (dd, J=5.2, 10.0 Hz, 1H), 5.06 (d, J=7.6 Hz, 1H), 4.91-4.79 (m 1H), 2.35 (s, 3H), 1.91 (s, 3H), 1.39 (d, J=6.4 Hz, 3H). MS (ESI): mass calcd. For C₂₆H₂₂BFN₂O₄ 456.28, m/z found 457.2 [M+H]⁺. HPLC: 99.16 (220 nm), 99.49 (254 nm). Chiral purity: 99% ee.

### Example 49

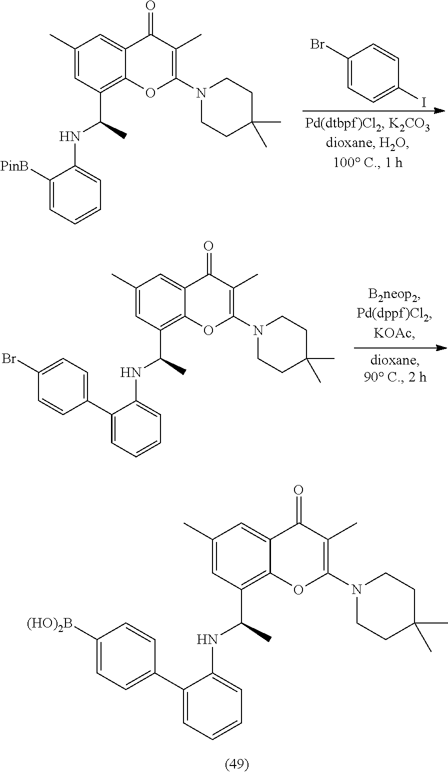

### Preparation of 8-[(1R)-1-[2-(4-bromophenyl)anilino]ethyl]-2-(4,4-dimethyl-1-piperidyl)-3,6-dimethyl-chromen-4-one

To a solution of 2-(4,4-dimethyl-1-piperidyl)-3,6-dimethyl-8-[(1R)-1-[2-(4,4,5,5-tetramethyl-1,3,2-dioxaborolan-2-yl)anilino]ethyl]chromen-4-one (1.00 g, 1.88 mmol, 1 eq) in dioxane (10 mL)/H₂O (1 mL) were added ditert-butyl(cyclopentyl)phosphane;dichloropalladium;iron (123 mg, 188.50 μmol, 0.1 eq), 1-bromo-4-iodo-benzene (587 mg, 2.07 mmol, 1.1 eq) and K₂CO₃ (521 mg, 3.77 mmol, 2 eq) at 25° C. in turns under N₂ atmosphere. The reaction mixture was heated to 100° C. and stirred at 100° C. for 1 h. The reaction mixture was filtered and the filtrate was purified by prep-HPLC (column: Welch Xtimate C18 180×70 mm×10 um; mobile phase: [H2O (10 mM □H4HCO3)-AC□]; gradient:65%-100% B over 22.0 min) to give the title compound (80 mg, 142.98 μmol, 7.58% yield) as a light yellow solid.

### Preparation of [4-[2-[[(1R)-1-[2-(4,4-dimethyl-1-piperidyl)-3,6-dimethyl-4-oxo-chrome n-8-yl]ethyl]amino]phenyl]phenyl]boronic acid

The title compound was prepared as a white solid (11 mg, 20.97 μmol, 16.77% yield) according to the procedures described herein and the synthetic scheme, using 8-[(1R)-1-[2-(4-bromophenyl)anilino]ethyl]-2-(4,4-dimethyl-1-piperidyl)-3,6-dimethyl-chromen-4-one (700 mg, 125 mol, 1 eq) and B₂neop₂ (113 mg, 500 μmol, 4 eq). ¹H NMR (DMSO-d₆, 400 MHz) δ 8.08 (s, 2H), 7.90 (d, J=7.6 Hz, 2H), 7.61 (s, 1H), 7.42 (d, J=8.0 Hz, 3H), 7.05-6.98 (m, 2H), 6.67 (t, J=8.8 Hz, 1H), 6.48 (d, J=8.0 Hz, 1H), 4.97 (t, J=6.8 Hz, 1H), 4.73 (d, J=6.8 Hz, 1H), 3.29-3.24 (m, 4H), 2.31 (s, 3H), 1.90 (s, 3H), 1.46 (d, J=6.8 Hz, 3H), 1.40 (s, 4H), 0.96 (s, 6H). MS (ESI): mass calcd. For C₃₂H₃₇BN₂O₄ 524.28, m/z found 525.3 [M+H]⁺. HPLC: 98.69% (220 nm), 98.98% (254 nm). Chiral purity: 98.78% ee.

### Example 50

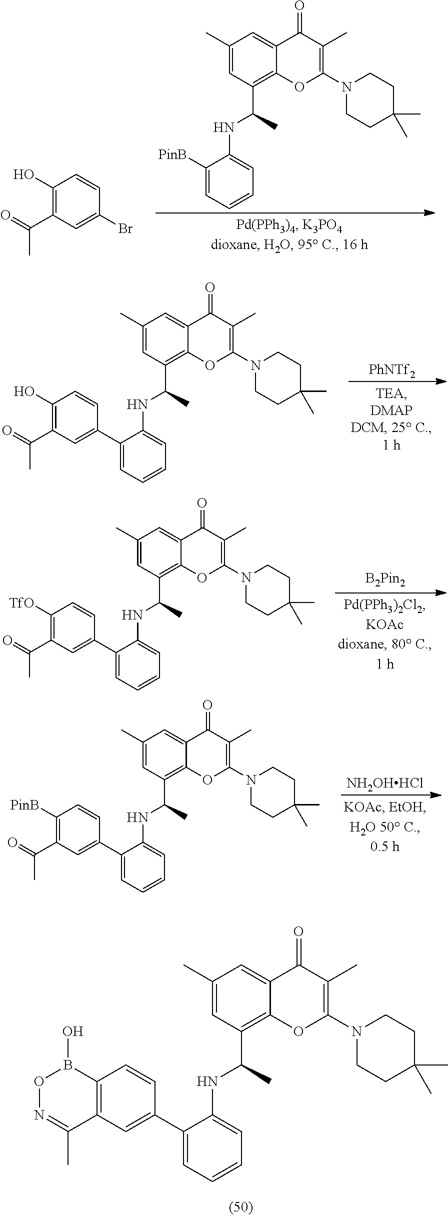

### Preparation of 8-[(1R)-1-[2-(3-acetyl-4-hydroxy-phenyl) anilino]ethyl]-2-(4,4-dimethyl-1-piperidyl)-3,6-dimethyl-chromen-4-one

A mixture of 2-(4,4-dimethyl-1-piperidyl)-3,6-dimethyl-8-[(1R)-1-[2-(4,4,5,5-tetramethyl-1,3,2-dioxaborolan-2-yl)anilino]ethyl]chromen-4-one (450 mg, 848 μmol, 1 eq) and 1-(5-bromo-2-hydroxy-phenyl)ethanone (182 mg, 848 μmol, 1 eq) in dioxane (4 mL)/H₂O (1 mL) were added K₃PO₄ (540 mg, 2.54 mmol, 3 eq) and Pd(PPh₃)₄ (98.0 mg, 84.8 μmol, 0.1 eq) in turns at 20° C., the reaction was degassed and purged with N₂ for 3 times. And then the reaction mixture was directly stirred at 95° C. (the oil-bath was pre-heated to 95° C.) for 16 h under N₂ atmosphere. The reaction was cooled to 25° C., filtered and concentrated in vacuo. The residue was purified by flash silica gel chromatography (BIOTAGE®; 20 g SepaFlash® Silica Flash Column, Eluent of 0~60% Ethyl acetate/Petroleum ether gradient@60 m/min) to give the title compound (350 mg, 649 μmol, 76.6% yield) as yellow oil. ¹H NMR (DMSO-d₆, 400 MHz) δ 11.98 (s, 1H), 7.95 (d, J=2.0 Hz, 1H), 7.72-7.58 (m, 2H), 7.47 (d, J=2.0 Hz, 1H), 7.08 (d, J=8.4 Hz, 1H), 7.03-7.01 (m, 2H), 6.67 (t, J=7.2 Hz, 1H), 6.52-6.46 (m, 1H), 5.07-4.89 (m, 2H), 3.44-3.34 (m, 4H), 2.65 (s, 3H), 2.32 (s, 3H), 1.91 (s, 3H), 1.52-1.47 (m, 3H), 1.46-1.37 (m, 4H), 0.977 (s, 6H).

### Preparation of [2-acetyl-4-[2-[[(1R)-1-[2-(4,4-dimethyl-1-piperidyl)-3,6-dimethyl-4-oxo-chromen-8-yl]ethyl]amino]phenyl]phenyl]trifluoromethanesulfonate

To a solution of 8-[(1R)-1-[2-(3-acetyl-4-hydroxy-phenyl)anilino]ethyl]-2-(4,4-dimethyl-1-piperidyl)-3,6-dimethyl-chromen-4-one (300 mg, 556 μmol, 1 eq), TEA (112 mg, 1.11 mmol, 155 μL, 2 eq) and DMAP (6.8 mg, 55.6 μmol, 0.1 eq) in DCM (5 mL) was added 1,1,1-trifluoro-N-phenyl-N-(trifluoromethylsulfonyl)methanesulfonamide (258 mg, 724 μmol, 1.3 eq) in turns at 25° C., the resulting reaction mixture was stirred at 25° C. for 1 h. The reaction mixture was quenched by adding water (20 mL) at 25° C., diluted with DCM (20 mL) and extracted with DCM (20 mL×3). The combined organic layers were washed with brine (50 mL), dried over Na₂SO₄, filtered and concentrated under reduced pressure. The residue was purified by flash silica gel chromatography (BIOTAGE®; 20 g SepaFlash® Silica Flash Column, Eluent of 0~30% Ethyl acetate/Petroleum ether gradient@60 mL/min) to give the title compound (370 mg, 551 μmol, 99.0% yield) as yellow oil. ¹H NMR (DMSO-d₆, 400 MHz) δ 8.26 (d, J=2.0 Hz, 1H), 7.90 (dd, J=2.4, 8.4 Hz, 1H), 7.63-7.56 (m, 2H), 7.48 (d, J=2.0 Hz, 1H), 7.15-7.03 (m, 2H), 6.71 (t, J=7.6 Hz, 1H), 6.48 (d, J=8.4 Hz, 1H), 5.36 (d, J=6.8 Hz, 1H), 5.14-4.88 (m, 1H), 3.45-3.35 (m, 4H), 2.65 (s, 3H), 2.31 (s, 3H), 1.92 (s, 3H), 1.51-1.47 (m, 3H), 1.47-1.40 (m, 4H), 0.98 (s, 6H).

### Preparation of 8-[(1R)-1-[2-[3-acetyl-4-(4,4,5,5-tetramethyl-1,3,2-dioxaborolan-2-yl) phenyl]anilino]ethyl]-2-(4,4-dimethyl-1-piperidyl)-3,6-dimethyl-chromen-4-one

To a mixture of [2-acetyl-4-[2-[[(1R)-1-[2-(4,4-dimethyl-1-piperidyl)-3,6-dimethyl-4-oxo-chromen-8-yl]ethyl]amino]phenyl]phenyl]trifluoromethanesulfonate (350 mg, 521 μmol, 1 eq), KOAc (102 mg, 1.04 mmol, 2 eq) and 4,4,5,5-tetramethyl-2-(4,4,5,5-tetramethyl-1,3,2-dioxaborolan-2-yl)-1,3,2-dioxaborolane (530 mg, 2.09 mmol, 4 eq) in dioxane (4 mL) was added Pd(PPh₃)₂Cl₂ (37 mg, 52.1 μmol, 0.1 eq) in one portion at 20° C., the reaction mixture was degassed and purged with N₂ for 3 times and stirred at 80° C. for 1 h under N₂ atmosphere. The reaction mixture was cooled to 20° C., filtered and the filtrate was concentrated under reduced pressure. The residue was purified by flash silica gel chromatography (BIOTAGE®; 20 g SepaFlash® Silica Flash Column, Eluent of 0~40% Ethyl acetate/Petroleum ether gradient@60 mL/min) to give the title compound (330 mg, 508 μmol, 97.5% yield) as yellow oil. ¹H NMR (DMSO-d₆, 400 MHz) δ 7.93 (s, 1H), 7.69 (dd, J=1.6, 7.2 Hz, 1H), 7.63-7.59 (m, 1H), 7.53 (d, J=7.2 Hz, 1H), 7.49 (d, J=2.0 Hz, 1H), 7.11-7.05 (m, 2H), 6.73-6.67 (m, 1H), 6.54 (d, J=8.4 Hz, 1H), 4.97 (s, 2H), 3.47-3.34 (m, 2H), 3.29-3.20 (m, 2H), 2.59-2.57 (m, 3H), 2.32 (s, 3H), 1.91 (s, 3H), 1.48 (d, J=6.0 Hz, 3H), 1.43-1.38 (m, 4H), 1.07 (s, 12H), 0.96 (s, 6H).

### Preparation of 2-(4,4-dimethyl-1-piperidyl)-8-[(1R)-1-[2-(1-hydroxy-4-methyl-2,3,1-benzoxazaborinin-6-yl)anilino]ethyl]-3,6-dimethyl-chromen-4-one

To a mixture of 8-[(1R)-1-[2-[3-acetyl-4-(4,4,5,5-tetramethyl-1,3,2-dioxaborolan-2-yl)phenyl]anilino]ethyl]-2-(4,4-dimethyl-1-piperidyl)-3,6-dimethyl-chromen-4-one (300 mg, 462 μmol, 1 eq) and NH₂OH·HCl (48.2 mg, 693 μmol, 1.5 eq) in EtOH (1 mL) and H₂O (1 mL) was added KOAc (68.0 mg, 693 μmol, 1.5 eq) in one portion at 20° C., and then the reaction mixture was stirred at 50° C. for 0.5 h. The resulting suspension was filtered and the filter cake washed with H₂O (5 mL). The solid was collected and directly purified by prep-HPLC (column: Waters Xbridge Prep OBD C18 150×40 mm×10 um; mobile phase: [H2O (10 mM NH4HCO3)-ACN]; gradient:40%-80% B over 8.0 min) to give the title compound (26 mg, 45.3 μmol, 9.81% yield) as a white solid. ¹H NMR (DMSO-d₆, 400 MHz) δ 9.28 (s, 1H), 8.16 (d, J=7.6 Hz, 1H), 7.86 (s, 1H), 7.82 (d, J=7.6 Hz, 1H), 7.61 (d, J=1.2 Hz, 1H), 7.48 (d, J=2.0 Hz, 1H), 7.16-7.04 (m, 2H), 6.72 (t, J=7.6 Hz, 1H), 6.58 (d, J=8.0 Hz, 1H), 5.14 (d, J=6.8 Hz, 1H), 5.01-4.84 (m, 1H), 3.30-3.20 (m, 4H), 2.41 (s, 3H), 2.32 (s, 3H), 1.90 (s, 3H), 1.47 (d, J=6.8 Hz, 3H), 1.45-1.35 (s, 4H), 0.95 (s, 6H). MS (ESI): mass calcd. For C₃₄H₃₈BN₃O₄ 563.30, m/z found 564.3 [M+H]⁺. HPLC: 98.37% (220 nm), 99.46% (254 nm). Chiral purity: 97.82 ee %

### Example 52

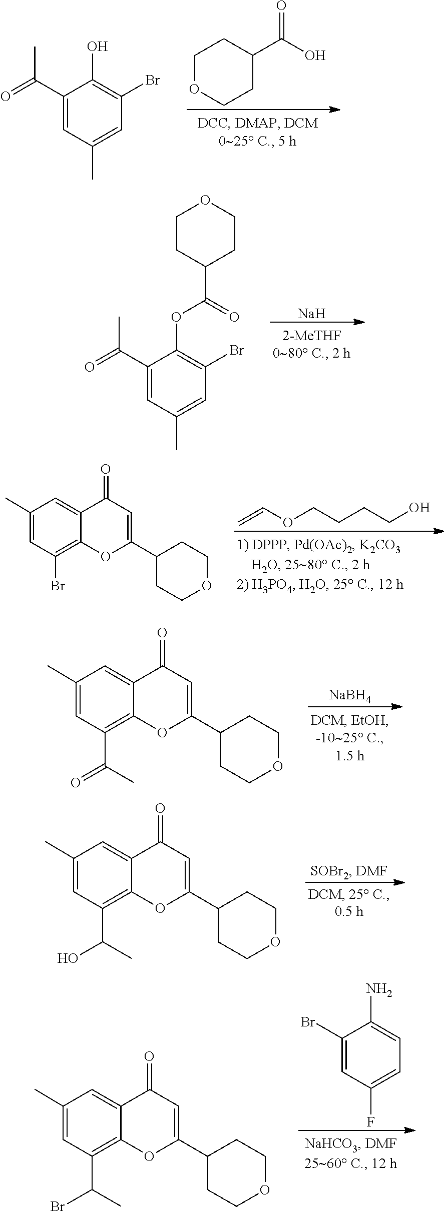

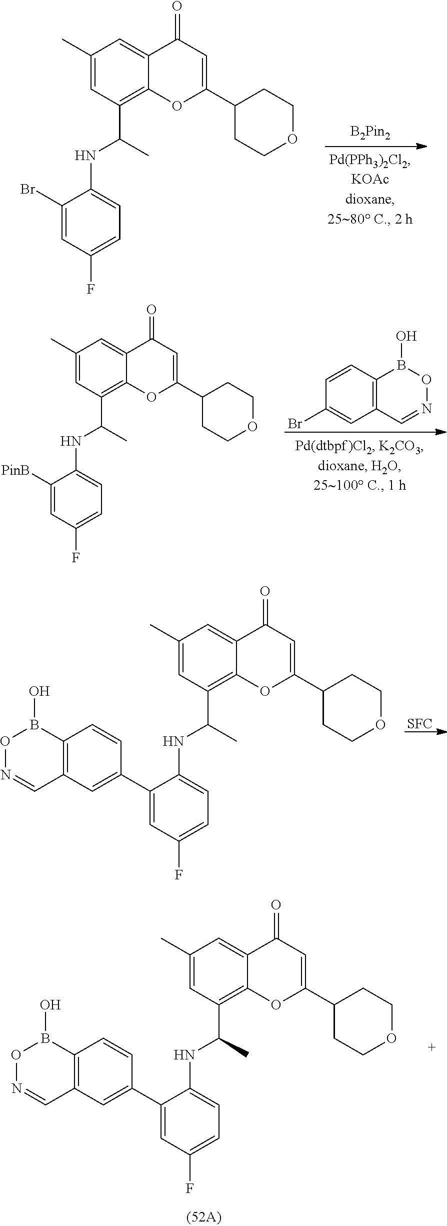

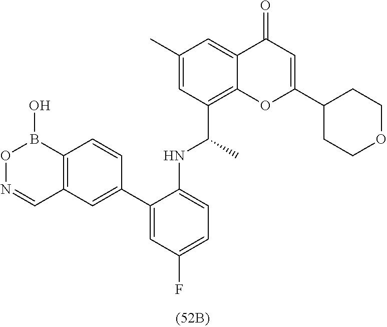 [SMILES: Cc1cc([C@H](C)Nc2ccc(F)cc2-c2ccc3c(c2)C=NOB3O)c2oc(C3CCOCC3)cc(=O)c2c1]

### Preparation of (2-acetyl-6-bromo-4-methyl-phenyl)tetrahydropyran-4-carboxylate

To a solution of 1-(3-bromo-2-hydroxy-5-methyl-phenyl)ethanone (15.0 g, 65.48 mmol, 1 eq), DMAP (2.4 g, 19.64 mmol, 0.3 eq) and tetrahydropyran-4-carboxylic acid (8.5 g, 65.48 mmol, 1 eq) in DCM (400 mL) was added a solution of DCC (21.6 g, 104.77 mmol, 21.2 mL, 1.6 eq) in DCM (100 mL) at 0° C. dropwise, then the reaction mixture was warmed up to 25° C. and stirred at 25° C. for 5 h. 2 parallel reactions were combined for work up. The reaction mixture was filtered and the filter cake was washed with DCM (200 mL). The filtrate was concentrated in vacuo and the residue was purified by flash silica gel chromatography (Biotage®; 330 g SepaFlash® Silica Flash Column, Eluent of 0~100% Ethyl acetate/Petroleum ether gradient@100 mL/min). Then the crude product was triturated with PE (200 mL) and dried in vacuo to give the title compound (28.1 g, 62.89% yield) as a white solid. ¹H NMR (CDCl₃, 400 MHz) δ 7.58 (s, 1H), 7.52 (s, 1H), 4.07-4.04 (m, 2H), 3.55-3.49 (m, 2H), 2.95-2.85 (m, 1H), 2.52 (s, 3H), 2.35 (s, 3H), 2.06-2.02 (m, 4H).

### Preparation of 8-bromo-6-methyl-2-tetrahydropyran-4-yl-chromen-4-one

To a solution of (2-acetyl-6-bromo-4-methyl-phenyl) tetrahydropyran-4-carboxylate (14.0 g, 41 mmol, 1 eq) in 2-MeTHF (140 mL) was added NaH (4.9 g, 123 mmol, 3 eq) at 0° C. portion-wise under N2 atmosphere, the mixture was heated to 80° C. and stirred at 80° C. for 2 h. 2 parallel reactions were combined for work up. The combined reaction mixture were cooled to 25° C., poured into sat.aq. NH₄Cl (400 mL) at 0° C., and then extracted with EtOAc (200 mL×3). The combined organic layers were washed with brine, dried over Na₂SO₄, filtered and concentrated under reduced pressure to give a residue. The residue was triturated with ACN (60 mL) to give the title compound (20.0 g, 75.41% yield) as a light yellow solid. ¹H NMR (DMSO-d₆, 400 MHz) δ 7.71 (s, 1H), 7.52 (s, 1H), 6.88 (s, 1H), 3.95-3.93 (m, 2H), 3.31-3.28 (m, 2H), 3.06 (d, J=16.0 Hz, 1H), 2.62 (d, J=16.0 Hz, 1H), 2.27 (s, 3H), 2.08-2.07 (m, 1H), 1.74 (d, J=11.6 Hz, 2H), 1.53-1.44 (m, 2H).

### Preparation of 8-acetyl-6-methyl-2-tetrahydropyran-4-yl-chromen-4-one

A mixture of 8-bromo-6-methyl-2-tetrahydropyran-4-yl-chromen-4-one (5.0 g, 15.47 mmol, 1 eq), 4-vinyloxybutan-1-ol (35.9 g, 309 mmol, 38.3 mL, 20 eq), K₂CO₃ (3.2 g, 23.2 mmol, 1.5 eq), 3-diphenylphosphanylpropyl(diphenyl)phosphane (1.3 g, 3.1 mmol, 0.2 eq) and Pd(OAc)₂ (347 mg, 1.55 mmol, 0.1 eq) in H₂O (30 mL) was degassed and purged with N₂ for 3 times at 25° C. under N₂ atmosphere, and then the mixture was heated to 80° C. and stirred at 80° C. for 2 h under N₂ atmosphere. 3 reactions were combined for work-up. The combined reactions were diluted with H₂O (100 mL) and extracted with EtOAc (100 mL×3). The combined organic layers were washed with brine (100 mL×2), dried over Na₂SO₄, filtered and concentrated under reduced pressure to give 8-[1-(4-hydroxybutoxy)vinyl]-6-methyl-2-tetrahy dropyran-4-yl-chromen-4-one (12 g, crude) as brown oil. To a solution of 8-[1-(4-hydroxybutoxy)vinyl]-6-methyl-2-tetrahydropyran-4-yl-chromen-4-one (3.0 g, 8.37 mmol, 1 eq) in H₂O (30 mL) was added H₃PO₄ (23.5 g, 240 mmol, 14 mL, 28.7 eq) dropwise at 25° C., the reaction mixture was stirred at 25° C. for 12 h. 4 parallel reactions were combined for work up. The reaction mixture was diluted with H₂O (200 mL) and extracted with EtOAc (200 mL×3). The combined organic layers were washed with brine (150 mL×2), dried over Na₂SO₄, filtered and concentrated under reduced pressure to give the title compound (10.2 g, 73.80% yield) as a yellow solid. ¹H NMR (CDCl₃, 400 MHz) δ 8.17 (s, 1H), 7.91 (s, 1H), 6.25 (s, 1H), 4.13 (d, J=12.0 Hz, 2H), 3.58-3.51 (m, 2H), 2.87-2.83 (m, 1H), 2.75 (s, 3H), 2.48 (s, 3H), 1.98-1.88 (m, 4H).

### Preparation of 8-(1-hydroxyethyl)-6-methyl-2-tetrahydropyran-4-yl-chromen-4-one

To a solution of 8-acetyl-6-methyl-2-tetrahydropyran-4-yl-chromen-4-one (9.2 g, 32.1 mmol, 1 eq) in DCM (50 mL)/EtOH (50 mL) was added NaBH₄ (3.5 g, 91.7 mmol, 2.9 eq) portion-wise at −10° C. under N₂ atmosphere, the reaction mixture stirred at −10° C. for 1 h. Then the reaction mixture was warmed up to 25° C. and stirred at 25° C. for 0.5 h. 2 parallel reactions were combined for work up. The reaction mixture was poured into sat.aq.NH₄Cl (200 mL) at 0° C. under N₂ atmosphere and then extracted with DCM (200 mL×3). The combined organic layers were washed with brine, dried over Na₂SO₄, filtered and concentrated in vacuo to give a residue. The residue was purified by Prep-HPLC (column: Welch Xtimate C18 180×70 mm×10 um; mobile phase: [H₂O (10 mM NH₄HCO₃)-AC□]; gradient:15%-45% B over 20 min) to give the title compound (4.1 g, 14.2 mmol, 44.2% yield) as a white solid. ¹H NMR (CDCl₃, 400 MHz) δ 7.89 (s, 1H), 7.68 (s, 1H), 6.19 (s, 1H), 5.49-5.44 (m, 1H), 4.12 (d, J=11.2 Hz, 2H), 3.58-3.51 (m, 2H), 2.84-2.83 (m, 1H), 2.46 (s, 3H), 1.92-1.85 (m, 4H), 1.59 (d, J=6.4 Hz, 3H).

### Preparation of 8-(1-bromoethyl)-6-methyl-2-tetrahydropyran-4-yl-chromen-4-one

To a solution of 8-(1-hydroxyethyl)-6-methyl-2-tetrahydropyran-4-yl-chromen-4-one (1.5 g, 5.2 mmol, 1 eq) and DMF (38 mg, 520 μmol, 40 μL, 0.1 eq) in DCM (15 mL) was added SOBr₂ (1.6 g, 7.8 mmol, 600 μL, 1.5 eq) dropwise at 25° C. under N₂ atmosphere, the mixture was stirred at 25° C. for 0.5 h. 2 parallel reactions were combined for work up. The reaction mixture was quenched with H₂O (100 mL), and then extracted with DCM (100 mL×3). The combined organic layers were washed dried over Na₂SO₄, filtered and concentrated under reduced pressure to give the title compound (2.68 g, crude) as a light yellow solid.

### Preparation of 8-[1-(2-bromo-4-fluoro-anilino)ethyl]-6-methyl-2-tetrahydropyran-4-yl-chromen-4-one

To a solution of 8-(1-bromoethyl)-6-methyl-2-tetrahydropyran-4-yl-chromen-4-one (2.0 g, 5.69 mmol, 1 eq) and 2-bromo-4-fluoro-aniline (1.41 g, 7.40 mmol, 1.3 eq) in DMF (20 mL) was added NaHCO₃ (956 mg, 11.39 mmol, 443 μL, 2 eq) in one portion at 25° C. under N₂ atmosphere, the reaction mixture was heated to 60° C. and stirred at 60° C. for 12 h. 3 reactions were combined for work up. The reaction mixture was quenched with H₂O (300 nm), and then extracted with EtOAc (100 mL×3). The combined organic layers were washed with brine, dried over Na₂SO₄, filtered and concentrated under reduced pressure to give a residue. The residue was purified by flash silica gel chromatography (Biotage®; 120 g SepaFlash® Silica Flash Column, Eluent of 0~100% Ethyl acetate/Petroleum ether gradient@100 mL/min) to give the title compound (2.8 g, 79.72% yield) as a light yellow solid. ¹H NMR (CDCl₃, 400 MHz) δ 7.88 (s, 1H), 7.45 (s, 1H), 7.50-7.22 (m, 1H), 6.76-6.74 (m, 1H), 6.23-6.20 (m, 2H), 5.03-4.99 (m, 1H), 4.14-4.10 (m, 2H), 3.58-3.51 (m, 2H), 2.80-2.90 (m, 1H), 2.38 (s, 3H), 1.96-1.91 (m, 4H), 1.66 (d, J=6.8 Hz, 3H).

### Preparation of 8-[1-[4-fluoro-2-(4,4,5,5-tetramethyl-1,3,2-dioxaborolan-2-yl)anilino]ethyl]-6-methyl-2-tetrahydropyran-4-yl-chromen-4-one

To a solution of 8-[1-(2-bromo-4-fluoro-anilino)ethyl]-6-methyl-2-tetrahydropyran-4-yl-chromen-4-one (1.1 g, 2.4 mmol, 1 eq) in dioxane (10 mL) was added 4,4,5,5-tetramethyl-2-(4,4,5,5-tetramethyl-1,3,2-dioxaborolan-2-yl)-1,3,2-dioxaborolane (1.8 g, 7.17 mmol, 3 eq), KOAc (586 mg, 5.97 mmol, 2.5 eq) and Pd(PPh₃)₂Cl₂ (168 mg, 239 μmol, 0.1 eq) at 25° C. under N₂ atmosphere. The reaction mixture was heated to 80° C. and stirred at 80° C. for 2 h. 4 reactions were combined for work up. The reaction mixture was cooled to room temperature and filtered. The filtrate was concentrated in vacuo and the residue was purified by flash silica gel chromatography (Biotage®; 120 g SepaFlash® Silica Flash Column, Eluent of 0~100% Ethyl acetate/Petroleum ether gradient@100 mL/min) to give the title compound (2.2 g, 71.28% yield) as a light yellow solid. ¹H NMR (CDCl₃, 400 MHz) δ 7.87 (s, 1H), 7.56 (s, 1H), 7.36-7.33 (m, 1H), 6.86-6.81 (m, 1H), 6.21-6.17 (m, 2H), 5.02-5.01 (m, 1H), 4.14-4.10 (m, 2H), 3.57-3.51 (m, 2H), 2.75-2.80 (m, 1H), 2.38 (s, 3H), 1.96-1.88 (m, 4H), 1.59 (d, J=6.4 Hz, 3H), 1.39 (s, 12H).

### Preparation of 8-[1-[4-fluoro-2-(1-hydroxy-2,3,1-benzoxazaborinin-6-yl)anilino]ethyl]-6-methyl-2-tetrahydropyran-4-yl-chromen-4-one

To a solution of 8-[1-[4-fluoro-2-(4,4,5,5-tetramethyl-1,3,2-dioxaborolan-2-yl)anilino]ethyl]-6-methyl-2-tetrahydropyran-4-yl-chromen-4-one (500 mg, 985 μmol, 1 eq), 6-bromo-1-hydroxy-2,3,1-benzoxazaborinine (222 mg, 985 μmol, 1 eq) and K2CO₃ (272 mg, 1.97 mmol, 2 eq) in dioxane (5 mL)/H₂O (0.5 mL) was added ditert-butyl (cyclopentyl)phosphane dichloropalladium iron (64 mg, 98 μmol, 0.1 eq) in one portion at 25° C. under N₂ atmosphere, the mixture was heated to 100° C. and stirred at 100° C. for 1 h. 2 reactions were combined for work up. The reaction mixture was cooled to room temperature and filtered. The filtrate was directly purified by Prep-HPLC (column: Waters Xbridge BEH C18 250×50 mm×10 um; mobile phase: [H₂O (10 mM NH₄HCO₃)-AC□]; gradient:25%-55% B over 10 min) to give the title compound (350 mg, 645 μmol, 48.2% yield) as a white solid.

### Preparation of 8-[(1S)-1-[4-fluoro-2-(1-hydroxy-2,3,1-benzoxazaborinin-6-yl)anilino]ethyl]-6-methyl-2-tetrahydropyran-4-yl-chromen-4-one and 8-[(R)-1-[4-fluoro-2-(1-hydroxy-2,3,1-benzoxazaborinin-6-yl)anilino]ethyl]-6-methyl-2-tetrahydropyran-4-yl-chromen-4-one

The compound 8-[1-[4-fluoro-2-(1-hydroxy-2,3,1-benzoxazaborinin-6-yl)anilino]ethyl]-6-methyl-2-tetrahydropyran-4-yl-chromen-4-one (350 mg, 665 μmol, 1 eq) was separated by SFC (column: ChiralPak IH, 250×30 mm, 10 um; mobile phase: [CO₂-EtOH]; B %:35%, isocratic elution mode) to give the title compound, Isomer 1 (121.3 mg, 230.4 μmol, 34.7% yield) as a white solid and the title compound, Isomer 2 (130.9 mg, 248.7 μmol, 37.4% yield) as a white solid. Isomer 1:

¹H NMR (DMSO-d₆, 400 MHz) δ 9.46 (s, 1H), 8.66 (s, 1H), 8.14 (d, J=7.6 Hz, 1H), 7.88-7.85 (m, 2H), 7.61-7.60 (m, 1H), 7.59-7.58 (m, 1H), 6.97-6.94 (m, 2H), 6.61-6.58 (m, 1H), 6.16 (s, 1H), 5.08-5.06 (d, J=7.6 Hz, 1H), 4.93-4.88 (m, 1H), 3.92-3.88 (m, 2H), 3.37-3.34 (m, 2H), 2.73-2.67 (m, 1H), 2.35 (s, 3H), 1.77-1.74 (m, 2H), 1.63-1.59 (m, 2H), 1.42 (d, J=6.4 Hz, 3H). MS (ESI): mass calcd. For C₃₀H₂₈BFN₂O₅ 526.37, m/z found 527.2 [M+H]⁺. HPLC: 99.32 (220 nm), 99.56 (254 nm). Chiral purity: 100 ee %. Isomer 2: ¹H NMR (DMSO-d₆, 400 MHz) δ 9.46 (s, 1H), 8.66 (s, 1H), 8.14 (d, J=7.6 Hz, 1H), 7.88-7.85 (m, 2H), 7.61-7.60 (m, 1H), 7.59-7.58 (m, 1H), 6.97-6.94 (m, 2H), 6.61-6.57 (m, 1H), 6.16 (s, 1H), 5.08-5.06 (d, J=7.6 Hz, 1H), 4.91-4.88 (m, 1H), 3.92-3.89 (m, 2H), 3.37-3.34 (m, 2H), 2.72-2.67 (m, 1H), 2.35 (s, 3H), 1.77-1.74 (m, 2H), 1.63-1.59 (m, 2H), 1.42 (d, J=6.4 Hz, 3H). MS (ESI): mass calcd. For C₃₀H₂₈BFN₂O₅ 526.37, m/z found 527.2 [M+H]¹. HPLC: 98.75 (220 nm), 98.21 (254 nm). Chiral purity: 99.78ee %

### Example 53

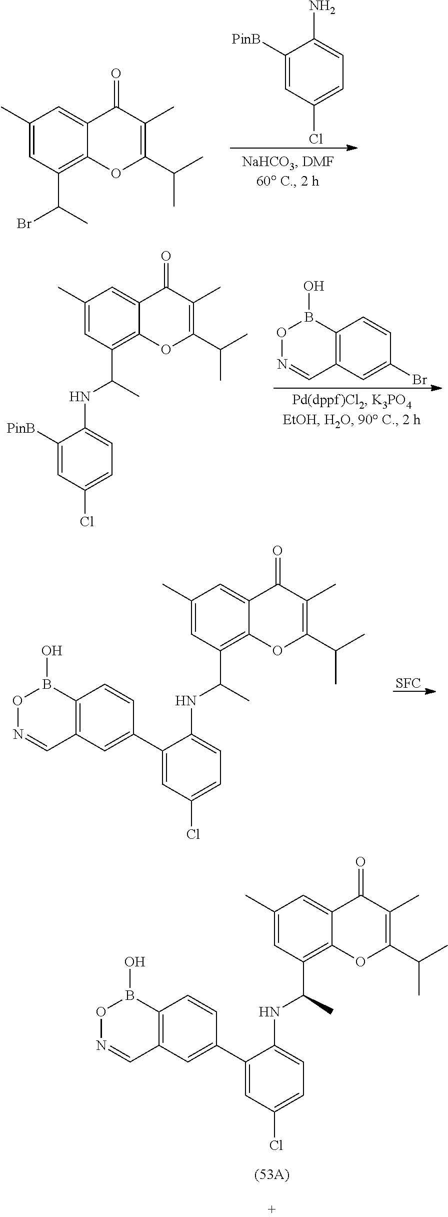

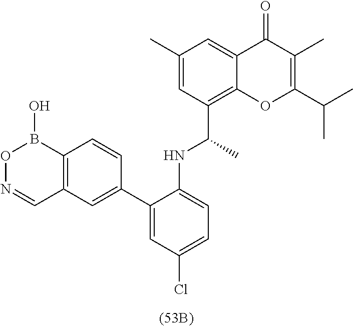 [SMILES: Cc1cc([C@H](C)Nc2ccc(Cl)cc2-c2ccc3c(c2)C=NOB3O)c2oc(C(C)C)c(C)c(=O)c2c1]

### Preparation of 8-(1-((4-chloro-2-(4,4,5,5-tetramethyl-1,3,2-dioxaborolan-2-yl)phenyl) amino)ethyl)-2-isopropyl-3,6-dimethyl-4H-chromen-4-one

A mixture of 8-(1-bromoethyl)-2-isopropyl-3,6-dimethyl-4H-chromen-4-one (1.50 g, 4.64 mmol, 1 eq), 4-chloro-2-(4,4,5,5-tetramethyl-1,3,2-dioxaborolan-2-yl)aniline (1.41 g, 5.57 mmol, 1.2 eq) and NaHCO₃ (779 mg, 9.28 mmol, 361 μL, 2 eq) in DMF (20 mL) was degassed and purged with N₂ for 3 times at 20° C., and then the mixture was heated to 60° C. stirred at 60° C. for 2 h. The reaction mixture was quenched by H₂O (60 mL) at 20° C., and then filtered. The filter cake was washed with H₂O (10 mL×3), dried in vacuum to give the title compound (1.30 g, 2.62 mmol, 56.50% yield) as a yellow solid, which was used for next step directly without further purification. MS (ESI): mass calcd. 495.23, m/z found 496.2 [M+H]⁺.

### Preparation of 8-(1-((4-chloro-2-(1-hydroxy-1H-benzo[d][1,2,6]oxazaborinin-6-yl) phenyl)amino)ethyl)-2-isopropyl-3,6-dimethyl-4H-chromen-4-one

A mixture of 8-(1-((4-chloro-2-(4,4,5,5-tetramethyl-1,3,2-dioxaborolan-2-yl)phenyl)amino) ethyl)-2-isopropyl-3,6-dimethyl-4H-chromen-4-one (700 mg, 1.41 mmol, 1 eq), 6-bromo-1H-benzo[d][1,2,6]oxazaborinin-1-ol (478 mg, 2.12 mmol, 1.5 eq), K₃PO₄ (599 mg, 2.82 mmol, 2 eq) and Pd(dppf)Cl₂ (103 mg, 141 μmol, 0.1 eq) in EtOH (7 mL)/H₂O (1 mL) was degassed and purged with N₂ for 3 times at 20° C., and then the mixture was heated to 90° C. and stirred at 90° C. for 2 h under N₂ atmosphere. The reaction mixture was filtered and the filtrate was concentrated under reduced pressure to give a residue. The residue was purified by prep-HPLC (column: Welch Xtimate C18 250×70 mm×10 um; mobile phase: [H2O (10 mM □H₄HCO₃)-AC□]; gradient:30%-70% B over 20.0 min) to give the title compound (230 mg, 446 μmol, 31.65% yield) as a white solid. MS (ESI): mass calcd. 514.18, m/z found 515.1 [M+H]⁺.

### Preparation of (S)-8-(1-((4-chloro-2-(1-hydroxy-1H-benzo[d][1,2,6]oxazaborinin-6-yl) phenyl)amino)ethyl)-2-isopropyl-3,6-dimethyl-4H-chromen-4-one & (R)-8-(1-((4-chloro-2-(1-hydroxy-1H-benzo[d][1,2,6]oxazaborinin-6-yl)phenyl)amino)ethyl)-2-isopropyl-3,6-dimethyl-4H-chromen-4-one

8-(1-((4-chloro-2-(1-hydroxy-1H-benzo[d][1,2,6]oxazaborinin-6-yl)phenyl)amino)ethyl)-2-isopropyl-3,6-dimethyl-4H-chromen-4-one (230 mg, 446 μmol, 1 eq) was purified by SFC (column: DAICEL CHIRALPAK IG (250 mm×30 mm×10 um); mobile phase: [CO2-IPA (0.1% NH₃H₂O)]; B %:50%, isocratic elution mode) to give the title compound, Isomer 1 (91.4 mg, 173 μmol, 38.80% yield) as a white solid and the title compound, Isomer 2 (90.0 mg, 172 μmol, 38.65% yield) as a white solid. Isomer 1: ¹H NMR (DMSO-d₆, 400 MHz) δ 9.52-9.41 (m, 1H), 8.66-8.66 (m, 1H), 8.15-8.15 (m, 1H), 7.87-7.63 (m, 1H), 7.63-7.48 (m, 3H), 7.09-7.01 (m, 2H), 6.46-6.37 (m, 1H), 5.04-4.96 (m, 2H), 3.32-3.24 (m, 1H), 2.34-2.29 (m, 3H), 1.99-1.96 (m, 3H), 1.46-1.44 (m, 3H), 1.26-1.12 (m, 6H). MS (ESI): mass calcd. For C₂₉H₂₈BClN₂O₄ 514.81, m/z found 515.2 [M+H]⁺. HPLC: 97.63% (220 nm), 97.42% (254 nm). Chiral purity: 100 ee %. Isomer 2: ¹H NMR (DMSO-d₆, 400 MHz) δ 9.52-9.43 (m, 1H), 8.66-8.65 (m, 1H), 8.32-8.15 (m, 1H), 7.87-7.85 (m, 1H), 7.67-7.48 (m, 3H), 7.09-7.01 (m, 2H), 6.46-6.37 (m, 1H), 5.29-4.81 (m, 2H), 3.32-3.20 (m, 1H), 2.33-2.23 (m, 3H), 1.99-1.96 (m, 3H), 1.46-1.44 (m, 3H), 1.26-1.12 (m, 6H). MS (ESI): mass calcd. For C₂₉H₂₈BClN₂O₄ 514.81, m/z found 515.2 [M+H]⁺. HPLC: 98.77% (220 nm), 99.26% (254 nm). Chiral purity: 98.46 ee %.

### Example 54

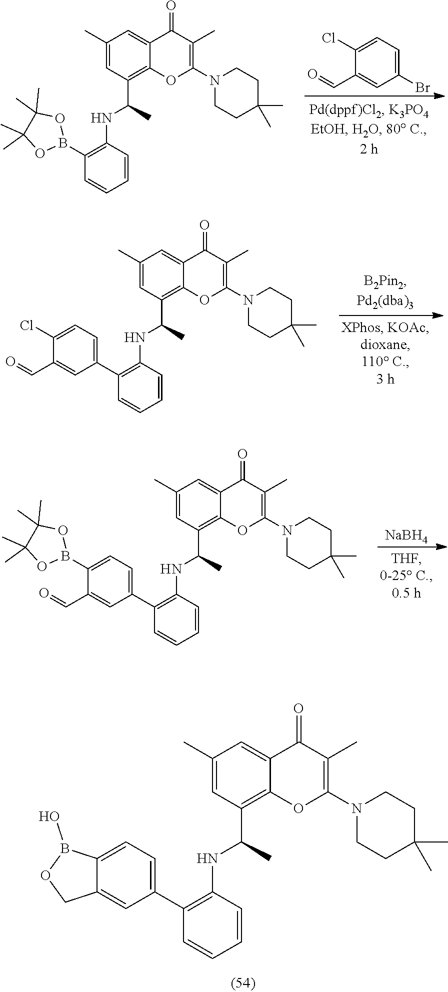

### Preparation of (R)-4-chloro-2′-((1-(2-(4,4-dimethylpiperidin-1-yl)-3,6-dimethyl-4-oxo-4H-chromen-8-yl)ethyl)amino)-[1,1′-biphenyl]-3-carbaldehyde

A mixture of 2-(4,4-dimethyl-1-piperidyl)-3,6-dimethyl-8-[(1R)-1-[2-(4,4,5,5-tetramethyl-1,3,2-dioxaborolan-2-yl)anilino]ethyl]chromen-4-one (460 mg, 867.10 μmol, 1 eq), 5-bromo-2-chloro-benzaldehyde (171 mg, 780.39 μmol, 0.9 eq), Pd(dppf)Cl₂ (64 mg, 86.71 μmol, 0.1 eq), K₃PO₄ (369 mg, 1.73 mmol, 2 eq) in EtOH (5 mL)/H₂O (1 mL) was degassed and purged with N₂ for 3 times in one portion at 25° C., and then the reaction mixture was heated to 80° C. and stirred at 80° C. for 2 h under N₂ atmosphere. The reaction mixture was cooled to 25° C., poured into H₂O (5 mL) and extracted with EtOAc (5 mL×3). The combined organic phase was washed with brine (5 mL×2), dried over Na₂SO₄, filtered and concentrated under reduced pressure. The residue was purified by flash silica gel chromatography (Biotage®; 12 g SepaFlash® Silica Flash Column, Eluent of 0-100% Ethyl acetate/Petroleum ether gradient@80 mL/min) to give the title compound (420 mg, 409.87 mol, 47.27% yield) as yellow oil. ¹H NMR (DMSO-d₆, 400 MHz) δ 10.35 (s, 1H), 7.93 (d, J=2.0 Hz, 1H), 7.82-7.79 (m, 1H), 7.70 (d, J=8.0 Hz, 1H), 7.60 (s, 1H), 7.45 (d, J=1.6 Hz, 1H), 7.08 (t, J=7.2 Hz, 1H), 7.03-7.01 (m, 1H), 6.69 (t, J=7.6 Hz, 1H), 6.54 (d, J=8.4 Hz, 1H), 5.17 (d, J=7.2 Hz, 1H), 4.96-4.91 (m, 1H), 3.37-3.33 (m, 4H), 2.32 (s, 3H), 1.92 (s, 3H), 1.45-1.43 (m, 7H), 0.98 (s, 6H).

### Preparation of (R)-2′-((1-(2-(4,4-dimethylpiperidin-1-yl)-3,6-dimethyl-4-oxo-4H-chromen-8-yl)ethyl)amino)-4-(4,4,5,5-tetramethyl-1,3,2-dioxaborolan-2-yl)-[1,1′-biphenyl]-3-carbaldehyde

A mixture of 2-chloro-5-[2-[[(1R)-1-[2-(4,4-dimethyl-1-piperidyl)-3,6-dimethyl-4-oxo-chromen-8-yl]ethyl]amino]phenyl]benzaldehyde (200 mg, 368.26 μmol, 1 eq), B₂Pin₂ (140 mg, 552.39 mol, 1.5 eq), KOAc (109 mg, 1.10 mmol, 3 eq), XPhos (7 mg, 14.73 μmol, 0.04 eq) and Pd₂(dba)₃ (4 mg, 3.68 μmol, 0.01 eq) in dioxane (2 mL) was degassed and purged with N₂ for 3 times in one portion at 25° C., and then the mixture was heated to 110° C. and stirred at 110° C. for 3 h under N₂ atmosphere. The reaction mixture was cooled to 25° C., poured into H₂O (3 mL) and extracted with EtOAc (3 mL×3). The combined organic phase was washed with brine (3 mL×2), dried over Na₂SO₄, filtered and concentrated under reduced pressure. The residue was purified by flash silica gel chromatography (Biotage®; 12 g SepaFlash® Silica Flash Column, Eluent of 0-100% Ethyl acetate/Petroleum ether gradient@36 mL/min) to give the title compound (260 mg, crude) as yellow oil, which was used directly for next step without further purification.

### Preparation of (R)-2-(4,4-dimethylpiperidin-1-yl)-8-(1-((2-(1-hydroxy-1,3-dihydrobenzo[c][1,2]oxaborol-5-yl)phenyl)amino)ethyl)-3,6-dimethyl-4H-chromen-4-one

To a solution of 5-[2-[[(1R)-1-[2-(4,4-dimethyl-1-piperidyl)-3,6-dimethyl-4-oxo-chromen-8-yl]ethyl]amino]phenyl]-2-(4,4,5,5-tetramethyl-1,3,2-dioxaborolan-2-yl)benzaldehyde (230 mg, 362.43 μmol, 1 eq) in THF (2.5 mL) was added NaBH₄ (80 mg, 2.11 mmol, 5.83 eq) in portions at 0° C. under N2. The reaction mixture was warmed to 25° C. and stirred at 25° C. for 30 min under N₂. The reaction mixture was quenched with 2 N HCl (3 mL) at 0° C. and extratcted with EtOAc (2 mL×2). The combined organic layers were washed with brine (2 mL), dried over Na₂SO₄ and concentrated under reduced pressure. The residue was purified by prep-HPLC (column: Waters Xbridge BEH C18 100×30 mm×10 um; mobile phase: [H₂O (10 mM NH₄HCO₃)-ACN]; gradient: 50%-80% B over 8.0 min) to give the title compound (2.4 mg, 4.47 μmol, 1.23% yield) as a white solid. ¹H NMR (DMSO-d₆, 400 MHz) δ 9.24 (s, 1H), 7.82 (d, J=8.0 Hz, 1H), 7.61 (d, J=1.2 Hz, 1H), 7.44-7.41 (m, 3H), 7.08-7.04 (m, 1H), 7.01-6.99 (m, 1H), 6.67 (t, J=7.6 Hz, 1H), 6.53 (d, J=8.0 Hz, 1H), 5.00 (s, 2H), 4.96-4.93 (m, 1H), 4.79 (d, J=6.8 Hz, 1H), 3.31-3.23 (m, 4H), 2.32 (s, 3H), 1.90 (s, 3H), 1.46 (d, J=6.8 Hz, 3H), 1.40-1.39 (m, 4H), 0.96 (s, 6H).

MS (ESI): mass calcd. For C₃₃H₃₇BN₂O₄ 536.48, m/z found 537.3 [M+H]⁺. HPLC: 99.26% (220 nm), 100.00% (254 nm). Chiral purity: 99.76% ee.

### Example 55

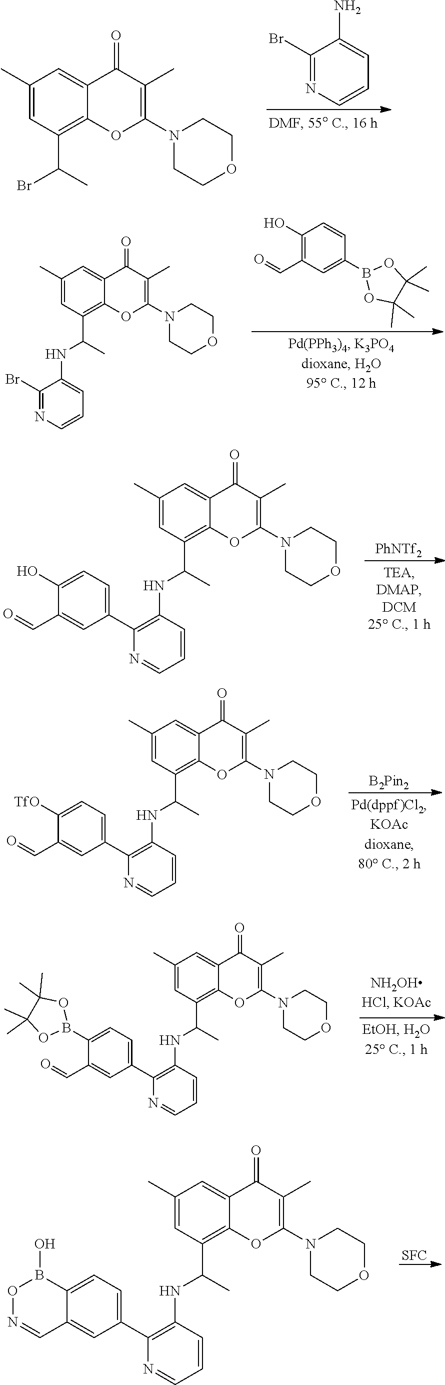

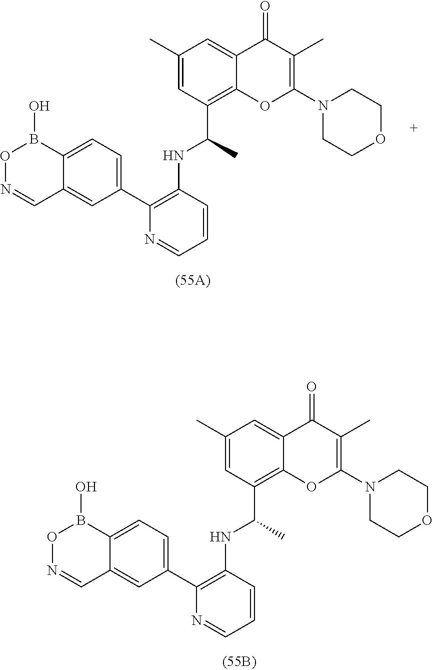 [SMILES: Cc1cc([C@@H](C)Nc2cccnc2-c2ccc3c(c2)C=NOB3O)c2oc(N3CCOCC3)c(C)c(=O)c2c1 ; Cc1cc([C@H](C)Nc2cccnc2-c2ccc3c(c2)C=NOB3O)c2oc(N3CCOCC3)c(C)c(=O)c2c1]

### Preparation of 8-[1-[(2-bromo-3-pyridyl)amino]ethyl]-3,6-dimethyl-2-morpholino-chromen-4-one

To a solution of 8-(1-bromoethyl)-3,6-dimethyl-2-morpholino-chromen-4-one (3.20 g, 8.74 mmol, 1 eq) in DMF (30 mL) was added 2-bromopyridin-3-amine (3.33 g, 19.22 mmol, 2.2 eq) at 25° C. The reaction mixture was warmed to 55° C. and stirred at 55° C. for 16 h. The reaction mixture was cooled to 25° C., poured into ice-water (90 mL) and extracted with EtOAc (10 mL×2). The combined organic layers were washed with brine (20 mL), dried over Na₂SO₄, filtered and concentrated under reduced pressure to give the residue. The crude product was triturated with Petroleum ether:Ethyl acetate (50 mL, v/v=3:1) to give the title compound (3.00 g, 6.55 mmol, 74.91% yield) as an off-white solid. ¹H NMR (DMSO-d₆, 400 MHz) δ 7.65 (s, 1H), 7.59 (d, J=4.8 Hz, 1H), 7.45 (d, J=2.0 Hz, 1H), 7.09 (dd, J=4.8, 8.0 Hz, 1H), 6.75 (d, J=8.0 Hz, 1H), 5.58 (d, J=7.2 Hz, 1H), 5.04 (q, J=6.8 Hz, 1H), 3.75-3.73 (m, 4H), 3.45-3.42 (m, 4H), 2.32 (s, 3H), 1.93 (s, 3H), 1.63 (d, J=6.8 Hz, 3H).

### Preparation of 5-[3-[1-(3,6-dimethyl-2-morpholino-4-oxo-chromen-8-yl)ethylamino]-2-pyridyl]-2-hydroxy-benzaldehyde

The title compound was prepared as light yellow oil (1.48 g, 2.96 mmol, 90.53% yield) according to the procedures described herein and the synthetic scheme, using 8-[1-[(2-bromo-3-pyridyl)amino]ethyl]-3,6-dimethyl-2-morpholino-chromen-4-one (1.50 g, 3.27 mmol, 1 eq) and 2-hydroxy-5-(4,4,5,5-tetramethyl-1,3,2-dioxaborolan-2-yl) benzaldehyde (812 mg, 3.27 mmol, 1 eq). ¹H NMR (CDCl₃, 400 MHz) δ 11.17 (s, 1H), 10.00 (s, 1H), 8.06 (dd, J=4.8 Hz, J=1.2 Hz, 1H), 7.99-7.98 (m, 1H), 7.95 (dd, J=8.4 Hz, J=2.0 Hz, 1H), 7.93-7.90 (m, 1H), 7.35 (d, J=2.0 Hz, 1H), 7.17 (d, J=8.8 Hz, 1H), 7.03 (q, J=4.8 Hz, 1H), 6.69 (d, J=8.0 Hz, 1H), 4.93-4.90 (m, 1H), 4.41 (d, J=4.8 Hz, 1H), 3.84-3.81 (m, 4H), 3.35-3.32 (m, 4H), 2.39 (s, 3H), 2.07 (s, 3H), 1.57 (d, J=6.8 Hz, 3H).

### Preparation of [4-[3-[1-(3,6-dimethyl-2-morpholino-4-oxo-chromen-8-yl)ethylamino]-2-pyridyl]-2-formyl-phenyl]trifluoromethanesulfonate

The title compound was prepared as a yellow solid (1.40 g, 1.99 mmol, 67.33% yield) according to the procedures described herein and the synthetic scheme, using 5-[3-[1-(3,6-dimethyl-2-morpholino-4-oxo-chromen-8-yl)ethylamino]-2-pyridyl]-2-hydroxy-benzaldehyde (1.48 g, 2.96 mmol).

¹H NMR (CHCl₃, 400 MHz) δ 10.35 (s, 1H), 8.44 (d, J=2.0 Hz, 1H), 8.13 (dd, J=10.8 Hz, J=8.8 Hz, 1H), 8.08 (d, J=3.6 Hz, 1H), 7.90 (s, 1H), 7.59 (d, J=8.4 Hz, 1H), 7.38 (d, J=1.6 Hz, 1H), 7.04 (q, J=4.8 Hz, 1H), 6.69 (d, J=8.0 Hz, 1H), 4.97-4.94 (m, 1H), 4.35 (d, J=5.2 Hz, 1H), 3.85-3.83 (m, 4H), 3.39-3.36 (m, 4H), 2.39 (s, 3H), 2.07 (s, 3H), 1.59 (d, J=6.8 Hz, 3H).

### Preparation of 5-[3-[1-(3,6-dimethyl-2-morpholino-4-oxo-chromen-8-yl)ethylamino]-2-pyridyl]-2-(4,4,5,5-tetramethyl-1,3,2-dioxaborolan-2-yl)benzaldehyde

The title compound was prepared as a light yellow solid (360 mg, 590.63 μmol, 63.77% yield) according to the procedure described in preceding examples, using [4-[3-[1-(3,6-dimethyl-2-morpholino-4-oxo-chromen-8-yl)ethylamino]-2-pyridyl]-2-formyl-phenyl]trifluoromethanesulfonate (650 mg, 926.19 μmol, 1 eq) and B₂Pin₂. ¹H NMR (CDCl₃, 400 MHz) δ 10.65 (s, 1H), 8.37 (s, 1H), 8.09 (dd, J=4.0 Hz, 1H), 7.90 (s, 1H), 7.71-7.65 (m, 1H), 7.50-7.45 (m, 1H), 7.34 (d, J=2.0 Hz, 1H), 7.10-7.06 (m, 1H), 6.74-6.72 (m, 1H), 4.95-4.88 (m, 1H), 4.56-4.51 (m, 1H), 3.83-3.80 (m, 4H), 3.35-3.26 (m, 4H), 2.40 (s, 3H), 2.06 (s, 3H), 1.55 (d, J=6.8 Hz, 3H), 1.44 (s, 12H).

### Preparation of 8-[1-[[2-(1-hydroxy-2,3,1-benzoxazaborinin-6-yl)-3-pyridyl]amino]ethyl]-3,6-dimethyl-2-morpholino-chromen-4-one

The title compound was prepared as a white solid (350 mg, 658.81 μmol, 66.93% yield) according to the procedures described herein and the synthetic scheme, using 5-[3-[1-(3,6-dimethyl-2-morpholino-4-oxo-chromen-8-yl)ethylamino]-2-pyridyl]-2-(4,4,5,5-tetramethyl-1,3,2-dioxaborolan-2-yl)benzaldehyde (600 mg, 984.38 μmol). ¹H NMR (DMSO-d₆, 400 MHz) δ 9.42 (s, 1H), 8.70 (s, 1H), 7.16 (d, J=7.6 Hz, 1H), 8.09-8.06 (m, 2H), 7.95 (dd, J=4.4 Hz, J=1.2 Hz, 1H), 7.63 (s, 1H), 7.51 (d, J=2.0 Hz, 1H), 7.09 (q, J=4.8 Hz, 1H), 6.92-6.90 (m, 1H), 5.57 (d, J=7.2 Hz, 1H), 4.96-4.93 (m, 1H), 3.67 (t, J=4.8 Hz, 4H), 3.28 (t, J=4.8 Hz, 4H), 2.32 (s, 3H), 1.91 (s, 3H), 1.50 (d, J=6.8 Hz, 3H).

### Preparation of 8-[(1S)-1-[[2-(1-hydroxy-2,3,1-benzoxazaborinin-6-yl)-3-pyridyl]amino]ethyl]-3,6-dimethyl-2-morpholino-chromen-4-one and 8-[(R)-1-[[2-(1-hydroxy-2,3,1-benzoxazaborinin-6-yl)-3-pyridyl]amino]ethyl]-3,6-dimethyl-2-morpholino-chromen-4-one

8-[1-[[2-(1-hydroxy-2,3,1-benzoxazaborinin-6-yl)-3-pyridyl]amino]ethyl]-3,6-dimethyl-2-morpholino-chromen-4-one (0.35 g, 667.46 μmol, 1 eq) was purified by SFC (column: DAICEL CHIRALCEL OD (250 mm×30 mm×10 um); mobile phase: [CO₂-EtOH (0.1% NH₃H₂O)]; gradient: 45%-5% B over 12.0 min) to give the title compound, Isomer 1 (130.9 mg, 247.64 μmol, 37.10% yield) as a white solid and the title compound, Isomer 2 (124.9 mg, 237.73 μmol, 35.62% yield) as a white solid. Isomer 1: ¹H NMR (DMSO-d₆, 400 MHz) δ 9.44 (s, 1H), 8.71 (s, 1H), 7.16 (d, J=7.6 Hz, 1H), 8.10-8.07 (m, 2H), 7.95 (dd, J=4.4 Hz, J=1.2 Hz, 1H), 7.63 (s, 1H), 7.51 (d, J=1.6 Hz, 1H), 7.09 (q, J=4.8 Hz, 1H), 6.91 (d, J=7.6 Hz, 1H), 5.58 (d, J=7.2 Hz, 1H), 4.98-4.93 (m, 1H), 3.67 (t, J=4.8 Hz, 4H), 3.29 (t, J=4.8 Hz, 4H), 2.32 (s, 3H), 1.91 (s, 3H), 1.50 (d, J=6.8 Hz, 3H). MS (ESI): mass calcd. For C₂₉H₂₉BN₄O₅ 524.22, m/z found 525.2 [M+H]⁺. HPLC: 99.20% (220 nm), 99.20% (254 nm). Chiral purity: 100% ee. Isomer 2: ¹H NMR (DMSO-d₆, 400 MHz) δ 9.43 (s, 1H), 8.71 (s, 1H), 7.17 (d, J=7.6 Hz, 1H), 8.10-8.07 (m, 2H), 7.95 (dd, J=4.4 Hz, J=1.2 Hz, 1H), 7.63 (s, 1H), 7.51 (d, J=2.4 Hz, 1H), 7.09 (q, J=4.8 Hz, 1H), 6.91 (d, J=7.6 Hz, 1H), 5.58 (d, J=7.2 Hz, 1H), 4.96-4.93 (m, 1H), 3.67 (t, J=4.8 Hz, 4H), 3.27 (t, J=4.8 Hz, 4H), 2.32 (s, 3H), 1.91 (s, 3H), 1.50 (d, J=6.8 Hz, 3H). MS (ESI): mass calcd. For C₂₉H₂₉BN₄O₅ 524.22, m/z found 525.3 [M+H]⁺. HPLC: 99.81% (220 nm), 100.00% (254 nm). Chiral purity: 96.54% ee.

### Example 56

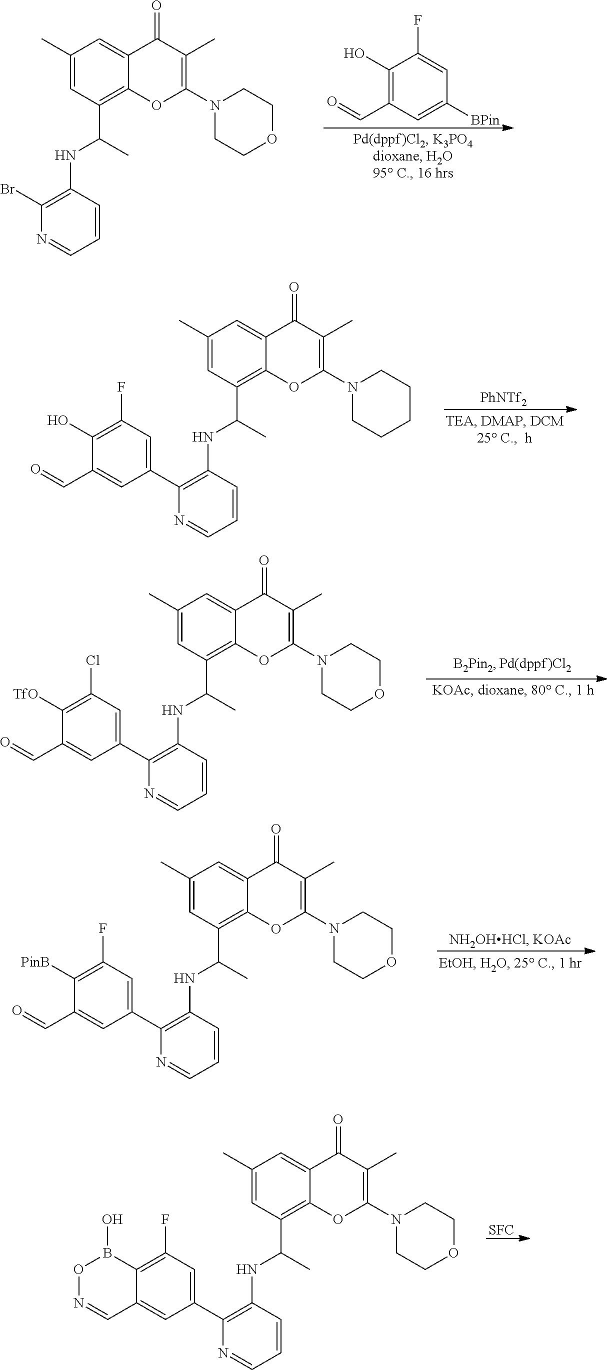

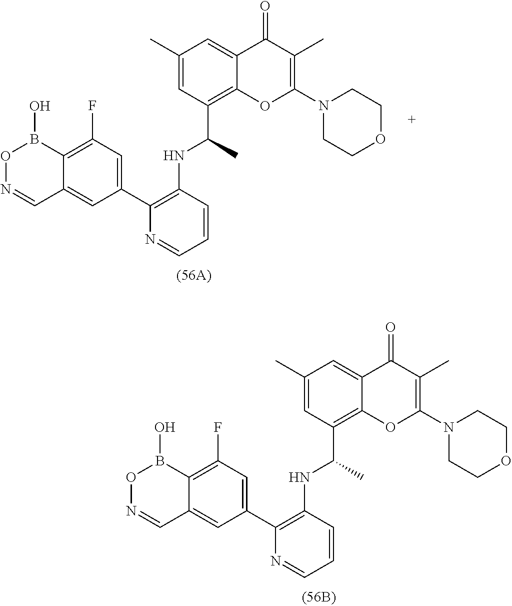 [SMILES: Cc1cc([C@@H](C)Nc2cccnc2-c2cc(F)c3c(c2)C=NOB3O)c2oc(N3CCOCC3)c(C)c(=O)c2c1 ; Cc1cc([C@H](C)Nc2cccnc2-c2cc(F)c3c(c2)C=NOB3O)c2oc(N3CCOCC3)c(C)c(=O)c2c1]

### Preparation of 5-[3-[1-(3,6-dimethyl-2-morpholino-4-oxo-chromen-8-yl)ethylamino]-2-pyridyl]-3-fluoro-2-hydroxy-benzaldehyde

The title compound was prepared as a yellow solid (1.10 g, 2.13 mmol, 57.30% yield) according to the procedures described herein and the synthetic scheme, using 8-[1-[(2-bromo-3-pyridyl)amino]ethyl]-3,6-dimethyl-2-morpholino-chromen-4-one (1.70 g, 3.71 mmol, 1 eq) and 3-fluoro-2-hydroxy-5-(4,4,5,5-tetramethyl-1,3,2-dioxaborolan-2-yl)benzaldehyde (1.97 g, 7.42 mmol, 2 eq). ¹H NMR (DMSO-d₆, 400 MHz) δ 11.11 (s, 1H), 10.33 (s, 1H), 7.91-7.84 (s, 3H), 7.63 (s, 1H), 7.49 (s, 1H), 7.04 (dd, J=4.8, 8.8 Hz, 1H), 6.85 (d, J=8.0 Hz, 1H), 5.60 (d, J=7.6 Hz, 1H), 4.96 (q, J=6.8 Hz, 1H), 3.74-3.70 (m, 4H), 3.34-3.33 (m, 4H), 2.32 (s, 3H), 1.92 (s, 3H), 1.50 (d, J=6.4 Hz, 3H).

### Preparation of [4-[3-[1-(3,6-dimethyl-2-morpholino-4-oxo-chromen-8-yl)ethylamino]-2-pyridyl]-2-fluoro-6-formyl-phenyl]trifluoromethanesulfonate

The title compound was prepared as a yellow solid (1.3 g, crude) according to the procedures described herein and the synthetic scheme, using 5-[3-[1-(3,6-dimethyl-2-morpholino-4-oxo-chromen-8-yl)ethylamino]-2-pyridyl]-3-fluoro-2-hydroxy-benzaldehyde (1.00 g, 1.93 mmol, 1 eq) and PhNTf₂ (1.38 g, 3.86 mmol, 2 eq). The product was used directly for next step without further purification. ¹H NMR (DMSO-d₆, 400 MHz) δ 10.14 (s, 1H), 8.31-8.29 (m, 1H), 8.20 (s, 1H), 7.96 (d, J=3.2 Hz, 1H), 7.64 (s, 1H), 7.50 (s, 1H), 7.13 (dd, J=4.4, 8.4 Hz, 1H), 6.86 (d, J=8.0 Hz, 1H), 6.11 (d, J=7.2 Hz, 1H), 4.98 (q, J=6.8 Hz, 1H), 3.75-3.72 (m, 4H), 3.41-3.39 (m, 4H), 2.31 (s, 3H), 1.91 (s, 3H), 1.54 (d, J=6.8 Hz, 3H).

### Preparation of 5-[3-[1-(3,6-dimethyl-2-morpholino-4-oxo-chromen-8-yl)ethylamino]-2-pyridyl]-3-fluoro-2-(4,4,5,5-tetramethyl-1,3,2-dioxaborolan-2-yl)benzaldehyde

The title compound was prepared as a yellow solid (0.7 g, crude) according to the procedure described in preceding examples, using [4-[3-[1-(3,6-dimethyl-2-morpholino-4-oxo-chromen-8-yl)ethylamino]-2-pyridyl]-2-fluoro-6-formyl-phenyl]trifluoromethanesulfonate (1.20 g, 1.85 mmol, 1 eq) and B₂Pin₂ (1.41 g, 5.54 mmol, 3 eq). The product was used directly for next step without further purification.

### Preparation of 8-[1-[[2-(8-fluoro-1-hydroxy-2,3,1-benzoxazaborinin-6-yl)-3-pyridyl]amino]ethyl]-3,6-dimethyl-2-morpholino-chromen-4-one

The title compound was prepared as an off-white solid (0.3 g, 534.48 μmol, 55.90% yield) according to the procedures described herein and the synthetic scheme, using 5-[3-[1-(3,6-dimethyl-2-morpholino-4-oxo-chromen-8-yl)ethylamino]-2-pyridyl]-3-fluoro-2-(4,4,5,5-tetramethyl-1,3,2-dioxaborolan-2-yl)benzaldehyde (0.6 g, 956.16 μmol, 1 eq) and hydroxylamine;hydrochloride (99 mg, 1.43 mmol, 1.5 eq).

### Preparation of 8-[(1S)-1-[[2-(8-fluoro-1-hydroxy-2,3,1-benzoxazaborinin-6-yl)-3-pyridyl]amino]ethyl]-3,6-dimethyl-2-morpholino-chromen-4-one and 8-[(1R)-1-[[2-(8-fluoro-1-hydroxy-2,3,1-benzoxazaborinin-6-yl)-3-pyridyl]amino]ethyl]-3,6-dimethyl-2-morpholino-chromen-4-one

The 8-[1-[[2-(8-fluoro-1-hydroxy-2,3,1-benzoxazaborinin-6-yl)-3-pyridyl]amino]ethyl]-3,6-dimethyl-2-morpholino-chromen-4-one (0.3 g) was separated by SFC (column: DAICEL CHIRALPAK IG (250 mm×30 mm×10 um); mobile phase: [CO2-IPA]; B %: 50%, isocratic elution mode) to give the title compound, Isomer 1 (125.6 mg, 228.26 μmol, 41.27% yield) as an off-white solid and the title compound, Isomer 2 (120.5 mg, 220.29 μmol, 39.83% yield) as an off-white solid. Isomer 1: ¹H NMR (DMSO-d₆, 400 MHz) δ 9.14 (br s, 1H), 8.72 (s, 1H), 7.95-7.90 (m, 2H), 7.71-7.70 (m, 1H), 7.62 (s, 1H), 7.51 (s, 1H), 7.11 (dd, J=4.8, 8.4 Hz, 1H), 6.91 (d, J=8.0 Hz, 1H), 5.77 (d, J=6.8 Hz, 1H), 4.95 (q, J=6.8 Hz, 1H), 3.76-3.64 (m, 4H), 3.38-3.36 (m, 4H), 2.32 (s, 3H), 1.91 (s, 3H), 1.52 (d, J=6.8 Hz, 3H). MS (ESI): mass calcd. For C₂₉H₂₈BFN₄O₅ 542.21, m/z found 543.3 [M+H]⁺. HPLC: 99.15% (220 nm), 99.46% (254 nm). Chiral purity: 98.6% ee. Isomer 2: ¹H NMR (DMSO-d₆, 400 MHz) δ 9.30 (br s, 1H), 8.73 (s, 1H), 7.95 (s, 2H), 7.78 (d, J=9.6 Hz, 1H), 7.63 (s, 1H), 7.51 (s, 1H), 7.11 (dd, J=4.8, 8.4 Hz, 1H), 6.91 (d, J=8.0 Hz, 1H), 5.78 (d, J=7.2 Hz, 1H), 4.95 (q, J=6.8 Hz, 1H), 3.71-3.68 (m, 4H), 3.38-3.36 (m, 4H), 2.32 (s, 3H), 1.91 (s, 3H), 1.52 (d, J=6.8 Hz, 3H). MS (ESI): mass calcd. For C₂₉H₂₈BFN₄O₅ 542.21, m/z found 543.3 [M+H]⁺. HPLC: 98.57% (220 nm), 98.74% (254 nm). Chiral purity: 99.86% ee.

### Example 57

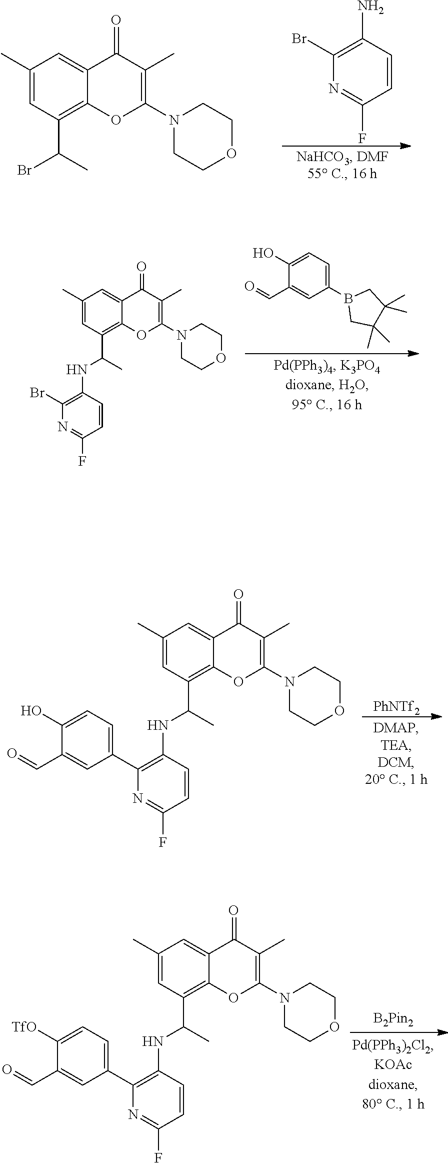

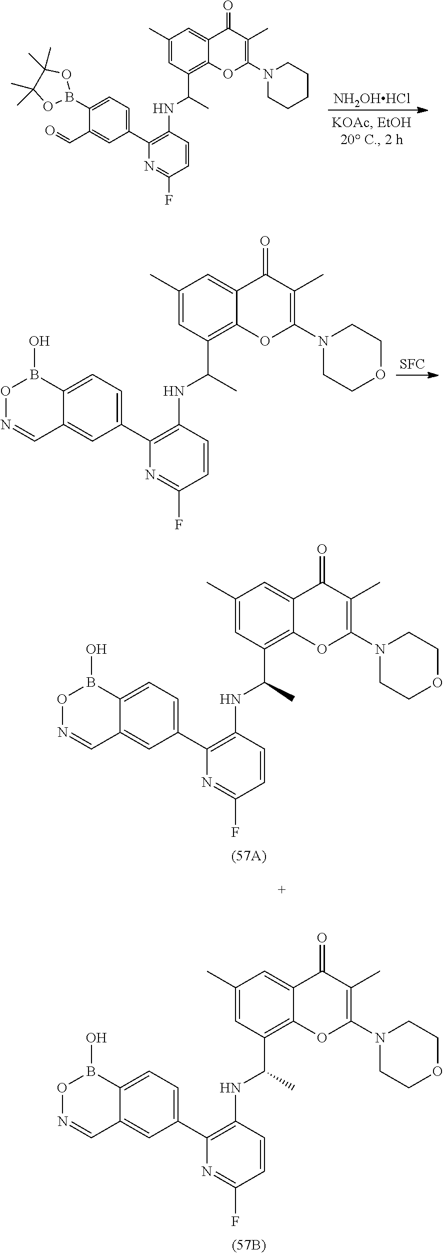

### Preparation of 8-(1-((2-bromo-6-fluoropyridin-3-yl)amino)ethyl)-3,6-dimethyl-2-morpholino-4H-chromen-4-one

A mixture of 8-(1-bromoethyl)-3,6-dimethyl-2-morpholino-4H-chromen-4-one (1.50 g, 4.10 mmol, 1 eq), 2-bromo-6-fluoropyridin-3-amine (1.17 g, 6.14 mmol, 1.5 eq) and NaHCO₃ (688 mg, 8.19 mmol, 2 eq) in DMF (10 mL) was degassed and purged with N₂ for 3 times at 20° C., and then the mixture was heated to 55° C. stirred at 55° C. for 16 h under N₂ atmosphere. The reaction mixture was quenched by H₂O (30 mL) at 20° C., and then filtered to the title compound (1.20 g, 2.52 mmol, 61.51% yield) as a white solid. ¹H NMR (CDCl₃, 400 MHz) δ 7.91 (d, J=1.2 Hz, 1H), 7.34 (d, J=2.4 Hz, 1H), 6.65-6.57 (m, 2H), 4.92-4.87 (m, 1H), 4.68-4.59 (m, 1H), 3.88-3.86 (m, 4H), 3.44-3.42 (m, 4H), 2.38 (s, 3H), 2.08 (s, 3H), 1.70-1.68 (m, 3H).

### Preparation of 5-(3-((1-(3,6-dimethyl-2-morpholino-4-oxo-4H-chromen-8-yl)ethyl)amino)-6-fluoropyridin-2-yl)-2-hydroxybenzaldehyde

The title compound was prepared as a yellow solid (800 mg, 1.55 mmol, 92.04% yield) according to the procedures described herein and the synthetic scheme, using 8-(1-((2-bromo-6-fluoropyridin-3-yl)amino)ethyl)-3,6-dimethyl-2-morpholino-4H-chromen-4-one (800 mg, 1.68 mmol, 1 eq) and 2-hydroxy-5-(4,4,5,5-tetramethyl-1,3,2-dioxaborolan-2-yl)benzaldehyde (541 mg, 2.18 mmol, 1.3 eq). ¹H NMR (CDCl₃, 400 MHz) δ 11.17 (s, 1H), 9.98 (s, 1H), 8.00-7.99 (m, 1H), 7.91-7.90 (m, 1H), 7.70-7.68 (m, 1H), 7.34-7.33 (m, 1H), 7.16-7.14 (m, 1H), 6.67-6.66 (m, 1H), 6.66-6.64 (m, 1H), 4.89-4.84 (m, 1H), 3.83-3.81 (m, 4H), 3.35-3.31 (m, 4H), 2.39 (s, 3H), 2.07 (s, 3H), 1.57 (d, J=6.8 Hz, 3H).

### Preparation of 4-(3-((1-(3,6-dimethyl-2-morpholino-4-oxo-4H-chromen-8-yl)ethyl)amino)-6-fluoropyridin-2-yl)-2-formylphenyl trifluoromethanesulfonate

The title compound was prepared as a yellow solid (1.90 g, 2.92 mmol, 94.61% yield) according to the procedures described herein and the synthetic scheme, using 5-(3-((1-(3,6-dimethyl-2-morpholino-4-oxo-4H-chromen-8-yl)ethyl)amino)-6-fluoropyridin-2-yl)-2-hydroxybenzaldehyde (1.60 g, 3.09 mmol, 1 eq) and PhNTf₂ (2.21 g, 6.18 mmol, 2 eq). ¹H NMR (CDCl3, 400 MHz) δ 10.35 (s, 1H), 8.48 (d, J=2.4 Hz, 1H), 8.17 (dd, J=8.4 Hz 2.4 Hz, 1H), 7.91 (s, 1H), 7.60-7.58 (m, 1H), 7.36-7.36 (m, 1H), 6.81-6.79 (m, 1H), 6.72-6.69 (m, 1H), 5.30 (s, 1H), 4.93-4.88 (m, 1H), 3.85-3.83 (m, 4H), 3.38-3.36 (m, 4H), 2.39 (s, 3H), 2.07 (s, 3H), 1.59 (d, J=6.8 Hz, 3H).

### Preparation of 5-(3-((1-(3,6-dimethyl-2-morpholino-4-oxo-4H-chromen-8-yl)ethyl)amino)-6-fluoropyridin-2-yl)-2-(4,4,5,5-tetramethyl-1,3,2-dioxaborolan-2-yl)benzaldehyde

The title compound was prepared as a yellow solid (1.25 g, 1.99 mmol, 71.89% yield) according to the procedures described herein and the synthetic scheme, using 4-(3-((1-(3,6-dimethyl-2-morpholino-4-oxo-4H-chromen-8-yl)ethyl)amino)-6-fluoropyridin-2-yl)-2-formylphenyl trifluoromethanesulfonate (1.80 g, 2.77 mmol, 1 eq) and B₂Pin₂ (3.52 g, 13.8 mmol, 5 eq). ¹H NMR (CDCl₃, 400 MHz) δ 10.63 (s, 1H), 8.38 (d, J=1.6 Hz, 1H), 8.06-8.04 (m, 1H), 7.99-7.96 (m, 1H), 7.90 (s, 1H), 7.34 (d, J=2.0 Hz, 1H), 6.78-6.76 (m, 1H), 6.68-6.65 (m, 1H), 5.30 (s, 1H), 4.88-4.83 (m, 1H), 3.82-3.80 (m, 4H), 3.32-3.27 (m, 4H), 2.39 (s, 3H), 2.06 (s, 3H), 1.54 (d, J=6.8 Hz, 3H), 1.43 (s, 12H).

### Preparation of 8-(1-((6-fluoro-2-(1-hydroxy-JH-benzo[d][1,2,6]oxazaborinin-6-yl) pyridin-3-yl)amino)ethyl)-3,6-dimethyl-2-morpholino-4H-chromen-4-one

The title compound was prepared as a white solid (300 mg, 537.20 μmol, 48.16% yield) according to the procedures described herein and the synthetic scheme, using 5-(3-((1-(3,6-dimethyl-2-morpholino-4-oxo-4H-chromen-8-yl)ethyl)amino)-6-fluoropyridin-2-yl)-2-(4,4,5,5-tetramethyl-1,3,2-dioxaborolan-2-yl)benzaldehyde (700 mg, 1.12 mmol, 1 eq) and NH₂OH·HCl (155 mg, 2.23 mmol, 2 eq). ¹H NMR (DMSO-d₆, 400 MHz) δ 9.45 (s, 1H), 8.69 (s, 1H), 8.16-8.08 (m, 3H), 7.61 (s, 1H), 7.50 (s, 1H), 7.18-7.14 (m, 1H), 6.93-6.90 (m, 1H), 5.68-5.66 (m, 1H), 4.94-4.84 (m, 1H), 3.67-3.65 (m, 4H), 3.26-3.23 (m, 4H), 2.32 (s, 3H), 1.89 (s, 3H), 1.49 (d, J=6.8 Hz, 3H).

### Preparation of (S)-8-(1-((6-fluoro-2-(1-hydroxy-1H-benzo[d][1,2,6]oxazaborinin-6-yl) pyridin-3-yl)amino)ethyl)-3,6-dimethyl-2-morpholino-4H-chromen-4-one and (R)-8-(1-((6-fluoro-2-(1-hydroxy-1H-benzo[d][1,2,6]oxazaborinin-6-yl)pyridin-3-yl)amino)ethyl)-3,6-dimethyl-2-morpholino-4H-chromen-4-one

8-(1-((6-fluoro-2-(1-hydroxy-1H-benzo[d][1,2,6]oxazaborinin-6-yl)pyridin-3-yl)amino)ethyl)-3,6-dimethyl-2-morpholino-4H-chromen-4-one (300 mg, 553 μmol, 1 eq) was separated by SFC (column: DAICEL CHIRALCEL OD (250 mm×30 mm, 10 um); mobile phase: [CO2-EtOH]; B %: 40%, isocratic elution mode) to give the title compound, Isomer 1 (98.8 mg, 178 μmol, 32.33% yield) as a white solid and the title compound, Isomer 2 (97.7 mg, 179 μmol, 32.37% yield) as a white solid. Isomer 1: ¹H NMR (DMSO-d₆, 400 MHz) δ 9.47-9.42 (m, 1H), 8.66-8.66 (m, 1H), 8.29-8.14 (m, 2H), 8.08-8.06 (m, 1H), 7.62-7.60 (m, 1H), 7.49-7.46 (m, 1H), 7.01-6.81 (m, 2H), 5.66-5.43 (m, 1H), 4.97-4.86 (m, 1H), 3.73-3.67 (m, 4H), 3.32-3.25 (m, 4H), 2.32-2.28 (m, 3H), 1.90 (s, 3H), 1.50-1.46 (m, 3H). MS (ESI): mass calcd. For C₂₉H₂₈BFN₄O₅ 542.37, m/z found 543.1 [M+H]⁺. HPLC: 98.16% (220 nm), 98.79% (254 nm). Chiral purity: 100% ee. Isomer 2: ¹H NMR (DMSO-d₆, 400 MHz) δ 9.47-9.40 (m, 1H), 8.66-8.66 (m, 1H), 8.32-8.14 (m, 2H), 8.09-8.06 (m, 1H), 7.62-7.60 (m, 1H), 7.49-7.46 (m, 1H), 7.01-6.81 (m, 2H), 5.66-5.44 (m, 1H), 4.97-4.89 (m, 1H), 3.67-3.66 (m, 4H), 3.32-3.25 (m, 4H), 2.32-2.28 (m, 3H), 1.92-1.90 (m, 3H), 1.50-1.46 (m, 3H). MS (ESI): mass calcd. For C₂₉H₂₈BFN₄O₅ 542.37, m/z found 543.2 [M+H]⁺. HPLC: 99.41% (220 nm), 99.78% (254 nm). Chiral purity: 97.88% ee.

### Example 58

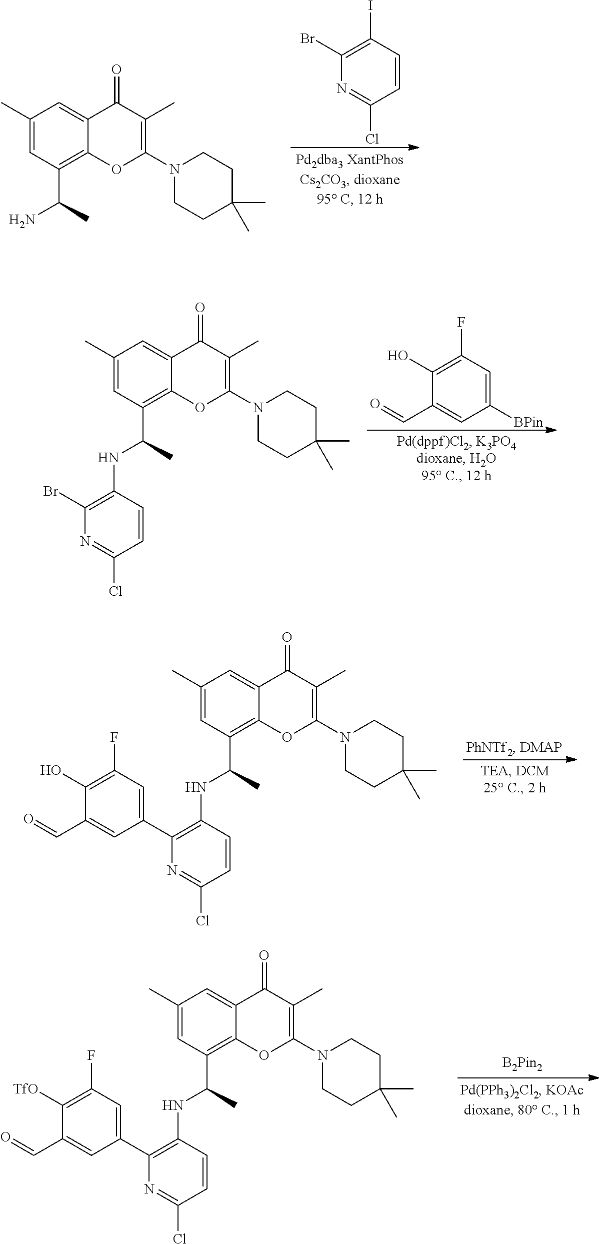

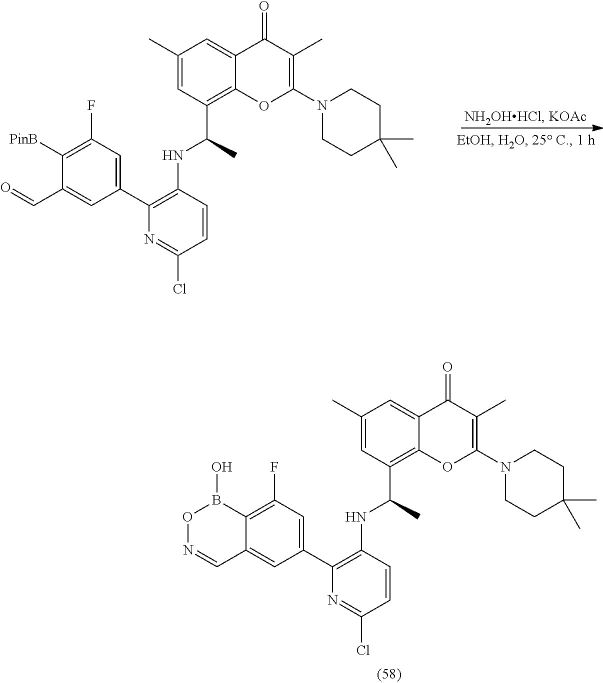

### Preparation of 8-[(1R)-1-[(2-bromo-6-chloro-3-pyridyl)amino]ethyl]-2-(4,4-dimethyl-1-piperidyl)-3,6-dimethyl-chromen-4-one

The title compound was prepared as a yellow solid (3.00 g, 5.78 mmol, 94.95% yield) according to the similar procedure in Example 78, using 8-[(1R)-1-aminoethyl]-2-(4,4-dimethyl-1-piperidyl)-3,6-dimethyl-chromen-4-one (2.00 g, 6.09 mmol, 1 eq) and 2-bromo-6-chloro-3-iodo-pyridine (3.88 g, 12.18 mmol, 2 eq). ¹H NMR (CDCl₃ 400 MHz) δ 7.89 (d, J=1.2 Hz, 1H), 7.28-7.27 (m, 1H), 6.96 (d, J=8.4 Hz, 1H), 6.48 (d, J=8.4 Hz, 1H), 4.94-4.88 (m, 1H), 4.81 (d, J=5.2 Hz, 1H), 3.41-3.36 (m, 4H), 2.37 (s, 3H), 2.05 (s, 3H), 1.69 (d, J=2.4 Hz, 3H), 1.54-1.51 (m, 4H), 1.04 (s, 6H).

### Preparation of 5-[6-chloro-3-[[(1R)-1-[2-(4,4-dimethyl-1-piperidyl)-3,6-dimethyl-4-oxo-chromen-8-yl]ethyl]amino]-2-pyridyl]-3-fluoro-2-hydroxy-benzaldehyde

The title compound was prepared as a yellow solid (1.50 g, crude) according to the procedures described herein and the synthetic scheme, using 8-[(1R)-1-[(2-bromo-6-chloro-3-pyridyl)amino]ethyl]-2-(4,4-dimethyl-1-piperidyl)-3,6-dimeth yl-chromen-4-one (1.50 g, 2.89 mmol, 1 eq) and 3-fluoro-2-hydroxy-5-(4,4,5,5-tetramethyl-1,3,2-dioxaborolan-2-yl) benzaldehyde (923 mg, 3.47 mmol, 1.2 eq). The product was used directly for next step without further purification.

### Preparation of (R)-4-(6-chloro-3-((1-(2-(4,4-dimethylpiperidin-1-yl)-3,6-dimethyl-4-oxo-4H-chromen-8-yl)ethyl)amino)pyridin-2-yl)-2-fluoro-6-formylphenyl trifluoromethane sulfonate

The title compound was prepared as a yellow solid (1.70 g, 2.39 mmol, 92.26% yield) according to the procedures described herein and the synthetic scheme, using (R)-5-(6-chloro-3-((1-(2-(4,4-dimethylpiperidin-1-yl)-3,6-dimethyl-4-oxo-4H-chromen-8-yl)ethyl)amino)pyridin-2-yl)-3-fluoro-2-hydroxybenzaldehyde (1.50 g, 2.59 mmol, 1 eq) and PhNTf₂ (1.24 g, 3.46 mmol, 1 eq). ¹H NMR (CDCl₃, 400 MHz) δ10.28 (d, J=1.2 Hz, 1H), 8.25-8.24 (m, 1H), 8.01 (dd, J=2.0 Hz, 8.0 Hz, 1H), 7.90-7.89 (m, 1H), 7.29 (t, J=2.0 Hz, 1H), 7.06 (d, J=8.4 Hz, 1H), 6.71 (d, J=8.8 Hz, 1H), 4.99-4.93 (m, 1H), 4.46 (d, J=4.8 Hz, 1H), 3.39-3.37 (m, 4H), 2.38 (s, 3H), 2.06 (s, 3H), 1.61 (d, J=6.8 Hz, 3H), 1.53-1.48 (m, 4H), 1.04 (s, 6H).

### Preparation of (R)-5-(6-chloro-3-((1-(2-(4,4-dimethylpiperidin-1-yl)-3,6-dimethyl-4-oxo-4H-chromen-8-yl)ethyl)amino)pyridin-2-yl)-3-fluoro-2-(4,4,5,5-tetramethyl-1,3,2-dioxaborolan-2-yl)benzaldehyde

The title compound was prepared as a yellow solid (1.00 g, crude) according to the procedure described in preceding examples, using [4-[6-chloro-3-[[(1R)-1-[2-(4,4-dimethyl-1-piperidyl)-3,6-dimethyl-4-oxo-chromen-8-yl]ethyl]amino]-2-pyridyl]-2-fluoro-6-formyl-phenyl]trifluorome thanesulfonate (1.30 g, 1.83 mmol, 1 eq) and B₂Pin₂ (2.32 g, 9.15 mmol, 5 eq). The product was used directly for next step without further purification.

### Preparation of 8-[(1R)-1-[[6-chloro-2-(8-fluoro-1-hydroxy-2,3,1-benzoxazaborinin-6-yl)-3-pyridyl]amino]ethyl]-2-(4,4-dimethyl-1-piperidyl)-3,6-dimethyl-chromen-4-one

The title compound was prepared as a white solid (106 mg, 175.82 μmol, 14.23% yield) according to the procedures described herein and the synthetic scheme, using 5-[6-chloro-3-[[(1R)-1-[2-(4,4-dimethyl-1-piperidyl)-3,6-dimethyl-4-oxo-chromen-8-yl]ethyl]amino]-2-pyridyl]-3-fluoro-2-(4,4,5,5-tetramethyl-1,3,2-dioxaborolan-2-yl)benzaldehyde (850 mg, 1.24 mmol, 1 eq) and NH₂OH·HCl (172 mg, 2.47 mmol, 2 eq). ¹H NMR (DMSO-d₆, +D₂O 400 MHz) δ 8.55 (s, 1H), 7.76 (s, 1H), 7.63 (d, J=9.2 Hz, 1H), 7.60 (s, 1H), 7.42 (d, J=2.0 Hz, 1H), 7.18 (d, J=8.4 Hz, 1H), 6.99 (dd, J=4 Hz, 4.8 Hz, 1H), 5.85 (d, J=6.4 Hz, 1H), 4.93-4.86 (m, 1H), 3.28 (s, 4H), 2.30 (s, 3H), 1.88 (s, 3H), 1.48 (d, J=6.4 Hz, 3H), 1.39-1.36 (m, 4H), 0.93 (s, 6H). MS (ESI): mass calcd. For C₃₂H₃₃BClFN₄O₄ 602.23, m/z found 603.1 [M+H]⁺. HPLC: 98.28% (220 nm), 99.37% (254 nm). Chiral purity: 100% ee.

### Example 59

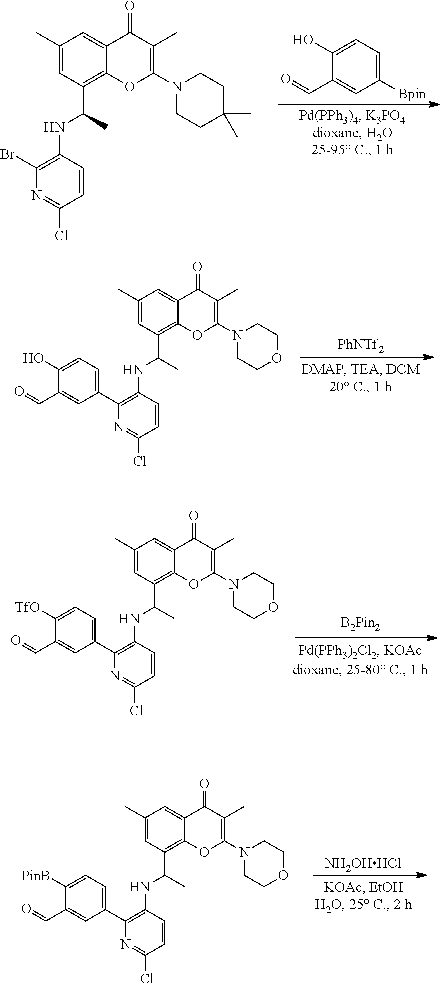

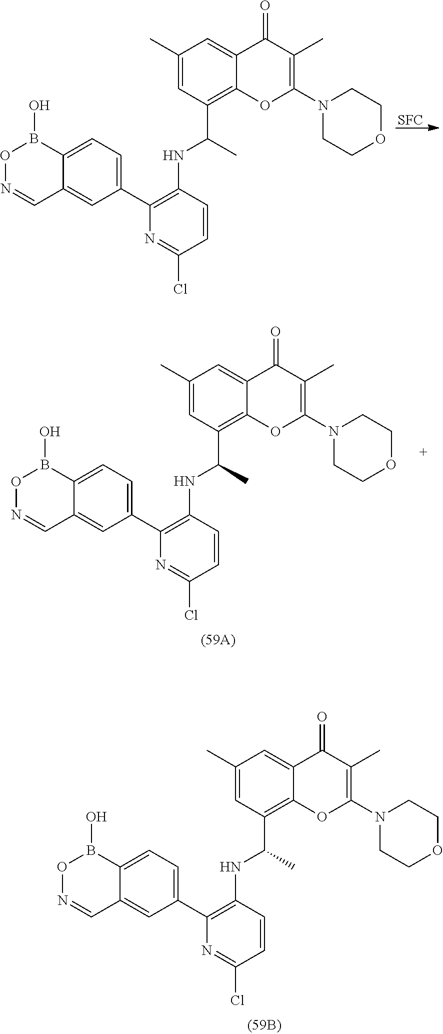

### Preparation of 5-(6-chloro-3-((1-(3,6-dimethyl-2-morpholino-4-oxo-4H-chromen-8-yl) ethyl)amino)pyridin-2-yl)-2-hydroxybenzaldehyde

To a solution of 8-[1-[(2-bromo-6-chloro-3-pyridyl)amino]ethyl]-3,6-dimethyl-2-morpholino-chromen-4-one (1.20 g, 2.44 mmol, 1 eq) in dioxane (15 mL)/H₂O (5 mL) were added 2-hydroxy-5-(4,4,5,5-tetramethyl-1,3,2-dioxaborolan-2-yl)benzaldehyde (725 mg, 2.92 mmol, 1.2 eq), K₃PO₄ (1.03 g, 4.87 mmol, 2 eq) and Pd(PPh₃)₄ (563 mg, 487 μmol, 0.2 eq) at 25° C. The reaction mixture was heated to 95° C. and stirred at 95° C. for 1 h. The reaction mixture was cooled to 25° C., diluted with H₂O (30 mL) and extracted with EtOAc (20 mL×3). The combined organic layers were washed with brine (20 mL×2), dried over Na₂SO₄, filtered and concentrated under reduced pressure. The residue was purified by flash silica gel chromatography (Biotage®; 40 g SepaFlash® Silica Flash Column, Eluent of 0~50% Ethyl acetate/Petroleum ether gradient @60 mL/min) to give the title compound (1.25 g, 2.34 mmol, 96.13% yield) as a brown solid. ¹H NMR (DMSO-d₆, 400 MHz) δ11.02 (s, 1H), 10.32 (s, 1H), 8.03 (d, J=2.4 Hz, 1H), 7.85 (dd, J=6.8, 2.4 Hz, 1H), 7.64 (s, 1H), 7.46 (s, 1H), 7.14-7.08 (m, 2H), 6.91 (d, J=6.8 Hz, 1H), 5.64 (d, J=7.2 Hz, 1H), 4.93 (t, J=7.2 Hz, 1H), 3.71 (t, J=4.8 Hz, 4H), 3.36-3.32 (m, 4H), 2.32 (s, 3H), 1.90 (s, 3H), 1.50 (d, J=6.4 Hz, 3H).

### Preparation of 4-(6-chloro-3-((1-(3,6-dimethyl-2-morpholino-4-oxo-4H-chromen-8-yl)ethyl)amino)pyridin-2-yl)-2-formylphenyl trifluoromethanesulfonate

A mixture of 5-[6-chloro-3-[l-(3,6-dimethyl-2-morpholino-4-oxo-chromen-8-yl)ethylamino]-2-pyridyl]-2-hydroxy-benzaldehyde (1.25 g, 2.34 mmol, 1 eq), 1,1,1-trifluoro-N-phenyl-N-(trifluoromethylsulfonyl)methanesulfonamide (1.67 g, 4.68 mmol, 2 eq), DMAP (28 mg, 234 μmol, 0.1 eq) and TEA (474 mg, 4.68 mmol, 652 μL, 2 eq) in DCM (30 mL) was degassed and purged with N₂ for 3 times at 20° C. Then the reaction mixture was stirred at 20° C. for 1 h under N₂ atmosphere. The reaction mixture was concentrated under reduced pressure and the residue was purified by flash silica gel chromatography (Biotage®; 40 g SepaFlash® Silica Flash Column, Eluent of 0~50% Ethyl acetate/Petroleum ether gradient @60 mL/min) to give the title compound (1.15 g, 1.73 mmol, 73.76% yield) as a yellow solid. ¹H NMR (DMSO-d₆, 400 MHz) δ10.15 (s, 1H), 8.48 (s, 1H), 8.27 (d, J=8.8 Hz, 1H), 7.72 (d, J=6.8 Hz, 1H), 7.57 (s, 1H), 7.47 (s, 1H), 7.19 (d, J=8.4 Hz, 1H), 6.93 (d, J=8.4 Hz, 1H), 6.16 (d, J=7.2 Hz, 1H), 4.96 (t, J=6.4 Hz, 1H), 3.73 (t, J=4.4 Hz, 4H), 3.41-3.38 (m, 4H), 2.32 (s, 3H), 1.93 (s, 3H), 1.52 (d, J=6.8 Hz, 3H).

### Preparation of 5-(6-chloro-3-((1-(3,6-dimethyl-2-morpholino-4-oxo-4H-chromen-8-yl) ethyl)amino)pyridin-2-yl)-2-(4,4,5,5-tetramethyl-1,3,2-dioxaborolan-2-yl)benzaldehyde

A mixture of [4-[6-chloro-3-[1-(3,6-dimethyl-2-morpholino-4-oxo-chromen-8-yl)ethylamino]-2-pyridyl]-2-formyl-phenyl]trifluoromethanesulfonate (1.10 g, 1.65 mmol, 1 eq), 4,4,5,5-tetramethyl-2-(4,4,5,5-tetramethyl-1,3,2-dioxaborolan-2-yl)-1,3,2-dioxaborolane (2.10 g, 8.26 mmol, 5 eq), KOAc (324 mg, 3.30 mmol, 2 eq) and Pd(PPh₃)₂Cl₂ (116 mg, 165 μmol, 0.1 eq) in dioxane (10 mL) was degassed and purged with N₂ for 3 times at 25° C. Then the reaction mixture was heated to 80° C. and stirred at 80° C. for 1 h under N₂ atmosphere. The reaction mixture was quenched by H₂O (20 mL) and then extracted with EtOAc (15 mL×3). The combined organic layers were washed with brine (10 mL×3), dried over Na₂SO₄, filtered and concentrated under reduced pressure to give a residue. The residue was purified by flash silica gel chromatography (Biotage®; 40 g SepaFlash® Silica Flash Column, Eluent of 0~50% Ethyl acetate/Petroleum ether gradient @60 mL/min) to give the title compound (800 mg, 1.24 mmol, 75.22% yield) as an off white solid. ¹H NMR (DMSO-d₆, 400 MHz) δ 10.42 (s, 1H), 8.22 (s, 1H), 8.04 (d, J=7.6 Hz, 1H), 7.87 (d, J=7.6 Hz, 1H), 7.63 (s, 1H), 7.46 (s, 1H), 7.17 (d, J=8.4 Hz, 1H), 6.98 (d, J=8.8 Hz, 1H), 5.79 (d, J=7.2 Hz, 1H), 4.94 (d, J=6.8 Hz, 1H), 3.69 (t, J=4.4 Hz, 4H), 3.29-3.26 (m, 4H), 2.32 (s, 3H), 1.91 (s, 3H), 1.49 (d, J=6.8 Hz, 3H), 1.37 (s, 12H).

### Preparation of 8-(1-((6-chloro-2-(1-hydroxy-1H-benzo[d][1,2,6]oxazaborinin-6-yl) pyridin-3-yl)amino)ethyl)-3,6-dimethyl-2-morpholino-4H-chromen-4-one

A mixture of 5-[6-chloro-3-[1-(3,6-dimethyl-2-morpholino-4-oxo-chromen-8-yl)ethylamino]-2-pyridyl]-2-(4,4,5,5-tetramethyl-1,3,2-dioxaborolan-2-yl)benzaldehyde (750 mg, 1.16 mmol, 1 eq), hydroxylamine;hydrochloride (162 mg, 2.33 mmol, 2 eq) and KOAc (228 mg, 2.33 mmol, 2 eq) in EtOH (10 mL)/H₂O (1 mL) was degassed and purged with N₂ for 3 times. Then the reaction mixture was stirred at 25° C. for 2 h under N₂ atmosphere. The reaction mixture was diluted with H₂O (10 mL) at 20° C. and then filtered. The filter cake was collected and dried in vacuo to give the title compound (350 mg, 582.48 μmol, 50.01% yield) as a white solid.

### Preparation of 8-[(1S)-1-[[6-chloro-2-(1-hydroxy-2,3,1-benzoxazaborinin-6-yl)-3-pyrid yl]amino]ethyl]-3,6-dimethyl-2-morpholino-chromen-4-one and 8-[(1R)-1-[[6-chloro-2-(1-hydroxy-2,3,1-benzoxazaborinin-6-yl)-3-pyridyl]amino]ethyl]-3,6-dimethyl-2-morpholino-chromen-4-one

The racemate was further separated by SFC (column: DAICEL CHIRALCEL OD (250 mm×30 mm×10 um); mobile phase: [CO2-IPA]; B %:50%, isocratic elution mode) to give the title compound, Isomer 1 (153.8 mg, 275.22 μmol, 43.94% yield) as a white solid and the title compound, Isomer 2 (154 mg, 276 μmol, 44.00% yield) as a white solid. Isomer 1: ¹H NMR (DMSO-d₆, 400 MHz) δ 9.45 (s, 1H), 8.72 (s, 1H), 8.17 (d, J=7.6 Hz, 1H), 8.06 (d, J=10.8 Hz, 2H), 7.63 (s, 1H), 7.47 (s, 1H), 7.19 (d, J=8.8 Hz, 1H), 6.98 (d, J=8.8 Hz, 1H), 5.83 (d, J=7.2 Hz, 1H), 4.93 (t, J=7.2 Hz, 1H), 3.68 (t, J=4.4 Hz, 4H), 3.28-3.25 (m, 4H), 2.32 (s, 3H), 1.91 (s, 3H), 1.50 (d, J=6.8 Hz, 3H). MS (ESI): mass calcd. For C₂₉H₂₈BClN₄O₅ 558.18, m/z found 559.1 [M+H]⁺. HPLC: 97.54% (220 nm), 97.15% (254 nm). Chiral purity: 100 ee %. Isomer 2: ¹H NMR (DMSO-d₆, 400 MHz) δ 9.46 (s, 1H), 8.72 (s, 1H), 8.17 (d, J=7.6 Hz, 1H), 8.06 (d, J=10.8 Hz, 2H), 7.63 (s, 1H), 7.47 (s, 1H), 7.19 (d, J=8.8 Hz, 1H), 6.98 (d, J=8.8 Hz, 1H), 5.83 (d, J=7.2 Hz, 1H), 4.93 (t, J=7.2 Hz, 1H), 3.68 (t, J=4.4 Hz, 4H), 3.28-3.25 (m, 4H), 2.32 (s, 3H), 1.91 (s, 3H), 1.50 (d, J=6.8 Hz, 3H). MS (ESI): mass calcd. For C₂₉H₂₈BClN₄O₅ 558.18, m/z found 559.1 [M+H]⁺. HPLC: 98.17% (220 nm), 98.45% (254 nm). Chiral purity: 99.12ee %.

### Example 60

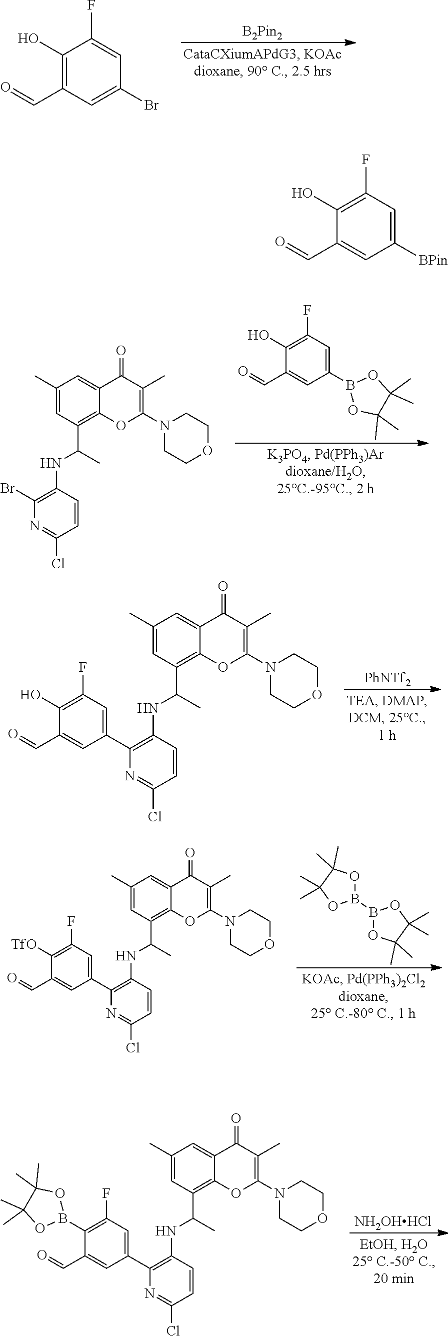

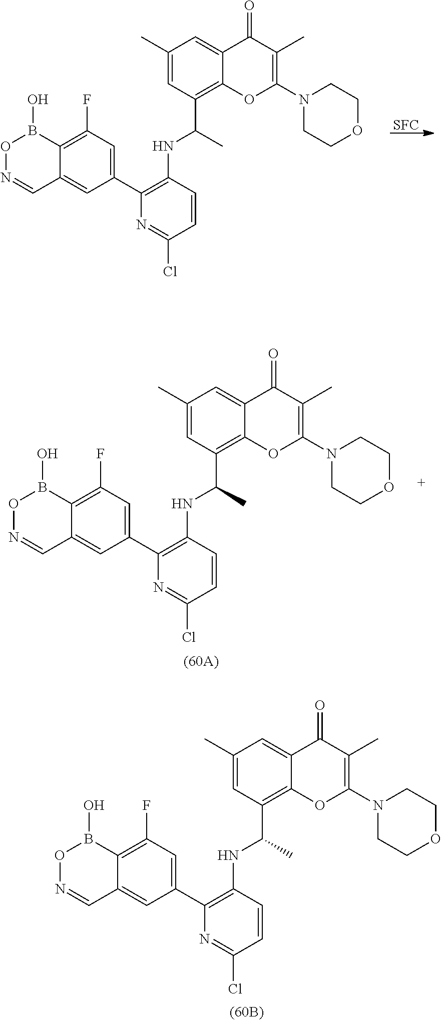

### Preparation of 3-fluoro-2-hydroxy-5-(4,4,5,5-tetramethyl-1,3,2-dioxaborolan-2-yl)benzaldehyde

A mixture of 5-bromo-3-fluoro-2-hydroxy-benzaldehyde (50.00 g, 228.30 mmol, 1 eq), 4,4,5,5-tetramethyl-2-(4,4,5,5-tetramethyl-1,3,2-dioxaborolan-2-yl)-1,3,2-dioxaborolane (86.96 g, 342.45 mmol, 1.5 eq), [2-(2-aminophenyl)phenyl]palladium(1+); bis(1-adamantyl)-butyl-phosphane;methanesulfonate (4.99 g, 6.85 mmol, 0.03 eq) and AcOK (67.22 g, 684.91 mmol, 3 eq) in dioxane (800 mL) was degassed and purged with N₂ for 3 times. The reaction mixture was heated to 90° C. and stirred at 90° C. for 2.5 h under N₂ atmosphere. The reaction mixture was cooled to 25° C., filtered through a pad of Celite gel and the filter cake was washed with EtOAc (100 mL×2). The filtrate was concentrated under reduced pressure and the residue was purified by flash silica gel chromatography (Biotage®; 330 g SepaFlash® Silica Flash Column, Eluent of 0~0% Ethyl acetate/Petroleum ether gradient @150 mL/min to give the title compound (62 g, crude) as a brown solid, which was used directly for next step without further purification. ¹H NMR (CDCl₃, 400 MHz) δ 11.20 (s, 1H), 9.95 (s, 1H), 7.85 (s, 1H), 7.74 (d, J=11.2 Hz, 1H), 1.35 (s, 12H).

### Preparation of 5-[6-chloro-3-[1-(3,6-dimethyl-2-morpholino-4-oxo-chromen-8-yl)ethylamino]-2-pyridyl]-3-fluoro-2-hydroxy-benzaldehyde

The title compound was prepared as a yellow solid (1.5 g, 2.72 mmol, 78.77% yield) according to the procedures described herein and the synthetic scheme, using 8-[1-[(2-bromo-6-chloro-3-pyridyl)amino]ethyl]-3,6-dimethyl-2-morpholino-chromen-4-one (1.7 g, 3.45 mmol, 1 eq) and 3-fluoro-2-hydroxy-5-(4,4,5,5-tetramethyl-1,3,2-dioxaborolan-2-yl)benzaldehyde (1.10 g, 4.14 mmol, 1.2 eq). ¹H NMR (DMSO-d₆, 400 MHz) δ11.24 (s, 1H), 10.35 (s, 1H), 7.92-7.80 (m, 2H), 7.65 (s, 1H), 7.48 (s, 1H), 7.14 (d, J=8.4 Hz, 1H), 6.92 (d, J=8.8 Hz, 1H), 5.85 (d, J=7.2 Hz, 1H), 4.99-4.94 (m, 1H), 3.74-3.72 (m, 4H), 3.51-3.28 (m, 4H), 2.34 (s, 3H), 1.94 (s, 3H), 1.52 (d, J=6.4 Hz, 3H).

### Preparation of [4-[6-chloro-3-[1-(3,6-dimethyl-2-morpholino-4-oxo-chromen-8-yl) ethylamino]-2-pyridyl]-2-fluoro-6-formyl-phenyl]trifluoromethanesulfonate

The title compound was prepared as a yellow solid (1.40 g, 2.05 mmol, 75.31% yield) according to the procedures described herein and the synthetic scheme, using 5-[6-chloro-3-[1-(3,6-dimethyl-2-morpholino-4-oxo-chromen-8-yl)ethylamino]-2-pyridyl]-3-fluoro-2-hydroxy-benzaldehyde (1.50 g, 2.72 mmol, 1 eq) and 1,1,1-trifluoro-N-phenyl-N-(trifluoromethylsulfonyl)methanesulfonamide (971 mg, 2.72 mmol, 1 eq). ¹H NMR (DMSO-d₆, 400 MHz) δ 10.16 (s, 1H), 8.34-8.30 (m, 2H), 7.66 (s, 1H), 7.47 (s, 1H), 7.22 (d, J=8.8 Hz, 1H), 6.94 (d, J=8.8 Hz, 1H), 6.31 (d, J=7.2 Hz, 1H), 5.00-4.98 (m, 1H), 4.05-3.74 (m, 4H), 3.50-3.30 (m, 4H), 2.34 (s, 3H), 1.95 (s, 3H), 1.55 (d, J=6.8 Hz, 3H).

### Preparation of 5-[6-chloro-3-[1-(3,6-dimethyl-2-morpholino-4-oxo-chromen-8-yl)ethylamino]-2-pyridyl]-3-fluoro-2-(4,4,5,5-tetramethyl-1,3,2-dioxaborolan-2-yl)benzaldehyde

The title compound was prepared as a yellow solid (1.4 g, 2.11 mmol, 96.45% yield) according to the procedures described herein and the synthetic scheme, using [4-[6-chloro-3-[1-(3,6-dimethyl-2-morpholino-4-oxo-chromen-8-yl)ethylamino]-2-pyridyl]-2-fluoro-6-formyl-phenyl]trifluoromethanesulfonate (1.50 g, 2.19 mmol, 1 eq) and B₂Pin₂ (2.23 g, 8.77 mmol, 4 eq).

### Preparation of 8-[1-[[6-chloro-2-(7-fluoro-1-hydroxy-2,3,1-benzoxazaborinin-6-yl)-3-pyridyl]amino]ethyl]-3,6-dimethyl-2-morpholino-chromen-4-one

The title compound was prepared as a yellow solid (0.90 g, 1.59 mmol, 88.23% yield) according to the procedures described herein and the synthetic scheme, using 5-[6-chloro-3-[1-(3,6-dimethyl-2-morpholino-4-oxo-chromen-8-yl)ethylamino]-2-pyridyl]-4-fluoro-2-(4,4,5,5-tetramethyl-1,3,2-dioxaborolan-2-yl)benzaldehyde (1.17 g, 1.77 mmol, 1 eq) and NH₂OH·HCl (123 mg, 1.77 mmol, 1 eq).

### Preparation of 8-[(1S)-1-[[6-chloro-2-(7-fluoro-1-hydroxy-2,3,1-benzoxazaborinin-6-yl)-3-pyridyl]amino]ethyl]-3,6-dimethyl-2-morpholino-chromen-4-one and 8-[(1R)-1-[[6-chloro-2-(7-fluoro-1-hydroxy-2,3,1-benzoxazaborinin-6-yl)-3-pyridyl]amino]ethyl]-3,6-dimethyl-2-morpholino-chromen-4-one

8-[1-[[6-chloro-2-(7-fluoro-1-hydroxy-2,3,1-benzoxazaborinin-6-yl)-3-pyridyl]amino]ethyl]-3,6-dimethyl-2-morpholino-chromen-4-one (500 mg) was separated by SFC (column: DAICEL CHIRALPAK IK (250 mm×25 mm, 10 um); mobile phase: [CO₂-EtOH]; B %:70%, isocratic elution mode) to give the title compound, Isomer 1 (234.1 mg, 398.74 μmol, 22.56% yield) as a white solid and the title compound, Isomer 2 (111.3 mg, 190.70 μmol, 10.79% yield) as a white solid. Isomer 1: ¹H NMR (DMSO-d₆, 400 MHz) δ 8.71 (s, 1H), 7.87 (s, 1H), 7.75 (d, J=9.6 Hz, 1H), 7.63 (s, 1H), 7.46 (s, 1H), 7.19 (d, J=8.4 Hz, 1H), 6.97 (d, J=8.8 Hz, 1H), 5.97 (d, J=6.8 Hz, 1H), 4.93 (t, J=6.8 Hz, 1H), 3.70 (t, J=4.4 Hz, 4H), 3.40-3.20 (m, 4H), 2.32 (s, 3H), 1.91 (s, 3H), 1.51 (d, J=6.8 Hz, 3H). MS (ESI): mass calcd. For C₂₉H₂₇BClFN₄O₅ 576.17, m/z found 577.2 [M+H]⁺. HPLC: 97.12% (220 nm), 98.90% (254 nm). Chiral purity: 100% ee (RT=1.356 min). Isomer 2: ¹H NMR (DMSO-d₆, 400 MHz) δ 8.71 (s, 1H), 7.87 (s, 1H), 7.75 (d, J=9.2 Hz, 1H), 7.63 (s, 1H), 7.46 (s, 1H), 7.19 (d, J=8.8 Hz, 1H), 6.97 (d, J=8.8 Hz, 1H), 5.97 (d, J=6.8 Hz, 1H), 4.93 (t, J=6.8 Hz, 1H), 3.70 (t, J=4.4 Hz, 4H), 3.40-3.30 (m, 4H), 2.32 (s, 3H), 1.91 (s, 3H), 1.51 (d, J=6.4 Hz, 3H). MS (ESI): mass calcd. For C₂₉H₂₇BClFN₄O₅ 576.17, m/z found 577.2 [M+H]⁺. HPLC: 99.41% (220 nm), 99.48% (254 nm). Chiral purity: 96.62% ee (RT=2.591 min).

### Example 61

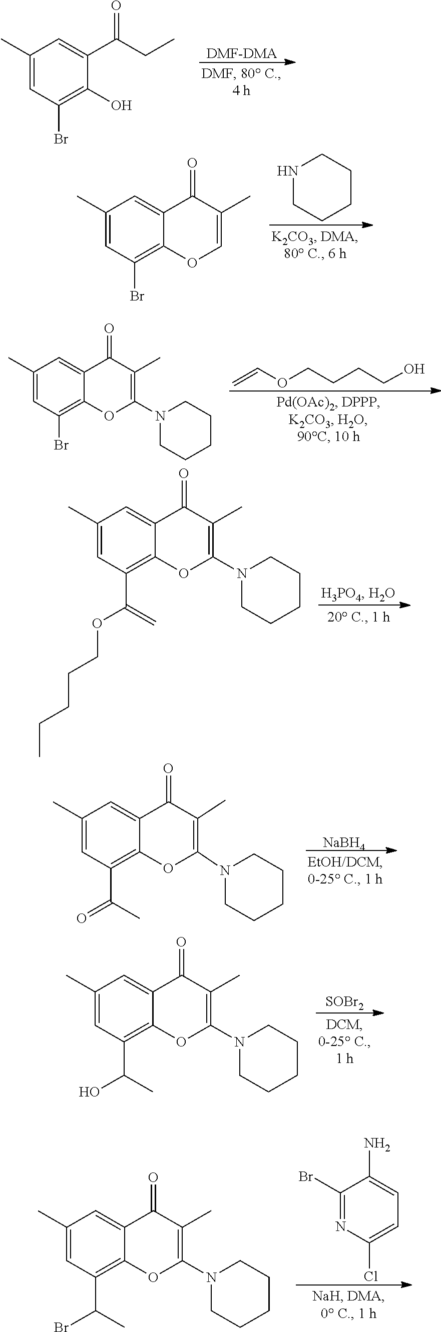

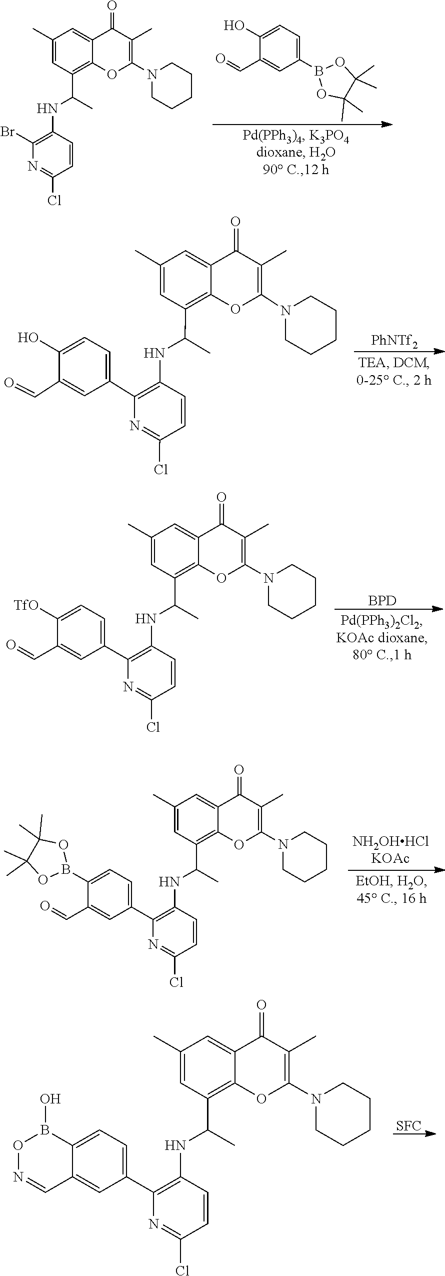

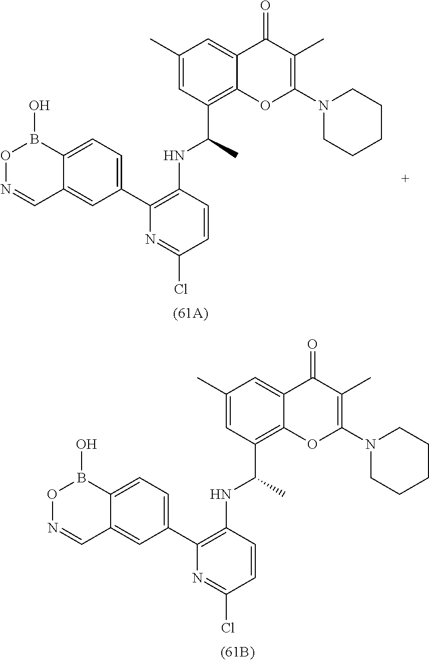 [SMILES: Cc1cc([C@@H](C)Nc2ccc(Cl)nc2-c2ccc3c(c2)C=NOB3O)c2oc(N3CCCCC3)c(C)c(=O)c2c1 ; Cc1cc([C@H](C)Nc2ccc(Cl)nc2-c2ccc3c(c2)C=NOB3O)c2oc(N3CCCCC3)c(C)c(=O)c2c1]

### Preparation of 8-bromo-3,6-dimethyl-chromen-4-one

To a solution of 1-(3-bromo-2-hydroxy-5-methyl-phenyl) propan-1-one (200.00 g, 822.72 mmol, 1 eq) in DMF (1.5 L) was added DMF-DMA (196.07 g, 1.65 mol, 218.58 mL, 2 eq) dropwise at 25° C. The reaction mixture was stirred at 80° C. for 4 h. 2 parallel reactions were combined for work up. The reaction mixture was poured into ice/H₂O (20 L) and the resulting suspension was filtered to give the title compound (380.00 g, 91.25% yield) as a yellow solid. ¹H NMR (DMSO-d₆, 400 MHz) δ 8.34 (d, J=1.2 Hz, 1H), 7.93 (d, J=2.0 Hz, 1H), 7.82 (s, 1H), 2.40 (s, 3H), 1.91 (d, J=0.8 Hz, 3H).

### Preparation of 8-bromo-3,6-dimethyl-2-(1-piperidyl)chromen-4-one

A mixture of 8-bromo-3,6-dimethyl-chromen-4-one (80.00 g, 316.09 mmol, 1 eq), piperidine (188.40 g, 2.21 mol, 218.51 mL, 7 eq), I₂ (200.57 g, 790.23 mmol, 159.18 mL, 2.5 eq), K₂CO₃ (218.43 g, 1.58 mol, 5 eq) and DMAP (3.86 g, 31.61 mmol, 0.1 eq) in DMA (800 ml) was degassed and purged with N₂ for 3 times, and then the reaction mixture was stirred at 80° C. for 6 h under N₂ atmosphere. 3 parallel reactions were combined for work up. The reaction mixture was cooled to 20° C., quenched by H₂O (7 L) at 0° C. and stirred for 5 min. The resulting suspension was filtered and the filter cake was washed with H₂O (1.5 L), dried in vacuo to afford the crude product. The crude product was further triturated with MTBE (1 L) at 25° C. for 30 min to give the title compound (240.00 g, 713.80 mmol, 75.27% yield) as a yellow solid. ¹H NMR (DMSO-d₆, 400 MHz) δ 7.90 (d, J=1.2 Hz, 1H), 7.58 (d, J=1.6 Hz, 1H), 3.47-3.46 (m, 4H), 2.41 (s, 3H), 2.03 (s, 3H), 1.75-1.70 (m, 6H).

### Preparation of 8-[1-(4-hydroxybutoxy)vinyl]-3,6-dimethyl-2-(1-piperidyl)chromen-4-one

A mixture of 8-bromo-3,6-dimethyl-2-(1-piperidyl)chromen-4-one (75.00 g, 223.07 mmol, 1 eq), 4-vinyloxybutan-1-ol (518.22 g, 4.46 mol, 551.88 mL, 20 eq), Pd(OAc)₂ (5.01 g, 22.31 mmol, 0.1 eq), DPPP (18.40 g, 44.61 mmol, 0.2 eq) and K₂CO₃ (46.24 g, 334.60 mmol, 1.5 eq) in H₂O (800 mL) was degassed and purged with N₂ for 3 times, and then the reaction mixture was stirred at 90° C. for 10 h under N₂ atmosphere. 2 parallel reactions were combined for work up. The reaction mixture was filtered and the filtrate was extracted with MTBE (1.5 L×2). The combined organic layers were washed with brine (2 L) and concentrated under reduced pressure to give the title compound (240 g, crude) as yellow oil, which was used directly for next step without further purification.

### Preparation of 8-acetyl-3,6-dimethyl-2-(1-piperidyl)chromen-4-one

To a solution of 8-[1-(4-hydroxybutoxy) vinyl]-3,6-dimethyl-2-(1-piperidyl)chromen-4-one (80.00 g, 215.36 mmol, 1 eq) in H₂O (1000 mL) was added H₃PO₄ (50 mL) dropwise at 20° C. The reaction mixture was stirred at 20° C. for 1 h. 3 parallel reactions were combined for work up. The resulting suspension was filtered. The filter cake was dissolved in DCM (2 L), washed with sat.aq.Na₂CO₃ to pH=8, dried over Na₂SO₄, filtered and the filtrate was concentrated in vacuo. The crude product was further triturated with MTBE (1 L) at 25° C. for 30 min to give the title compound (180.00 g, 601.28 mmol, 93.06% yield) as a yellow solid. ¹H NMR (DMSO-d₆, 400 MHz) δ 8.17 (s, 1H), 7.77 (d, J=0.2 Hz, 1H), 3.42-3.40 (m, 4H), 2.72 (s, 3H), 2.45 (s, 3H), 2.05 (s, 3H), 1.73-1.71 (m, 6H).

### Preparation of 8-(1-hydroxyethyl)-3,6-dimethyl-2-(piperidin-1-yl)-4H-chromen-4-one

To a solution of 8-acetyl-3,6-dimethyl-2-(1-piperidyl)chromen-4-one (48.0 g, 167.0 mmol, 1 eq) in DCM (250 mL)/EtOH (250 mL) was added NaBH₄ (10.9 g, 288.6 mmol, 1.8 eq) in portions at 0° C., the reaction mixture was stirred at 0°-25 C for 1 h. 2 parallel reactions were combined for work up. The reaction mixture was quenched by adding water (1 L) at 0° C., and extracted with DCM (200 mL×3). The combined organic layers were washed with brine (100 mL), dried over Na₂SO₄, filtered and concentrated under reduced pressure to give a residue. The crude product was triturated with MTBE (500 mL) at 25° C. for 30 min to give the title compound (96.0 g, 159.27 mmol, 99.3% yield) as a yellow solid. ¹H NMR (DMSO-d₆, 400 MHz) δ 7.63 (s, 1H), 7.58 (d, J=2.4 Hz, 1H), 5.33 (d, J=4.4 Hz, 1H), 5.24-5.20 (m, 1H), 3.38-3.68 (m, 4H), 2.40 (s, 3H), 1.90 (s, 3H), 1.66-1.61 (m, 6H), 1.41 (d, J=6.4 Hz, 3H).

### Preparation of 8-(1-bromoethyl)-3,6-dimethyl-2-(piperidin-1-yl)-4H-chromen-4-one

To a solution of 8-(1-hydroxyethyl)-3,6-dimethyl-2-(1-piperidyl)chromen-4-one (60.0 g, 199.1 mmol, 1 eq) and DMF (7.28 g, 99.5 mmol, 0.5 eq) in DCM (500 mL) was added sulfurous dibromide (62.07 g, 298.62 mmol, 1.5 eq) dropwise at 0° C., the mixture was stirred at 25° C. for 1 h. 2 parallel reactions were combined for work up. The reaction mixture was quenched by adding water (1 L) at 0° C., and extracted with DCM (100 mL×3). The combined organic layers were washed with brine (100 mL×2), dried over Na₂SO₄, filtered and concentrated under reduced pressure to give a residue. The crude product was further triturated with MTBE (1 L) at 25° C. for 30 min to give the title compound (120.0 g, 329.4 mmol, 82.73% yield) as a white solid. ¹H NMR (DMSO-d₆, 400 MHz) δ 7.76-7.70 (m, 2H), 5.85 (d, J=14.0, 7.2 Hz, 1H), 3.58-3.42 (m, 4H), 2.49 (s, 3H), 2.12 (d, J=6.8 Hz, 3H), 1.92 (s, 3H), 1.70-1.62 (m, 6H).

### Preparation of 8-[1-[(2-bromo-6-chloro-3-pyridyl)amino]ethyl]-3,6-dimethyl-2-(1-piperidyl)chromen-4-one

To a solution of 2-bromo-6-chloro-pyridin-3-amine (20.5 g, 98.82 mmol, 1.2 eq) in DMA (400 mL) was added NaH (9.88 g, 247.1 mmol, 3 eq) in portions at 0° C., the mixture was stirred at 0° C. for 10 min. Then a solution of 8-(1-bromoethyl)-3,6-dimethyl-2-(1-piperidyl)chromen-4-one (30.0 g, 82.35 mmol, 1 eq) in DMA (300 mL) was added dropwise to the above reaction mixture at 0° C., the resulting mixture was stirred at 0° C. for 1 h. 4 reactions were combined for work up. The reaction mixture was quenched by adding water (1000 mL) at 0° C., and extracted with DCM (300 mL×3). The combined organic layers were washed with brine (100 mL×2), dried over Na₂SO₄, filtered and concentrated under reduced pressure to give a residue. The crude product was triturated with MTBE (1200 mL) at 25° C. for 30 min to give the title compound (80.0 g, 162.9 mmol, 59.37% yield) as a yellow solid. ¹H NMR (CDCl₃, 400 MHz) δ 7.91 (s, 1H), 7.28 (s, 1H), 6.98 (d, J=7.6 Hz, 1H), 6.48 (d, J=7.6 Hz, 1H), 4.98-4.87 (m, 1H), 4.86-4.79 (m, 1H), 3.42-3.32 (m, 4H), 2.38 (s, 3H), 2.08 (s, 3H), 1.75-1.68 (m, 9H).

### Preparation of 5-[6-chloro-3-[1-[3,6-dimethyl-4-oxo-2-(1-piperidyl)chromen-8-yl]ethylamino]-2-pyridyl]-2-hydroxy-benzaldehyde

To a solution of 8-[1-[(2-bromo-6-chloro-3-pyridyl)amino]ethyl]-3,6-dimethyl-2-(1-piperidyl) chromen-4-one (35.00 g, 71.31 mmol, 1 eq) and 2-hydroxy-5-(4,4,5,5-tetramethyl-1,3,2-dioxaborolan-2-yl)benzaldehyde (23.00 g, 92.70 mmol, 1.3 eq) in dioxane (350 mL)/H₂O (60 mL) were added Pd(PPh₃)₄ (8.24 g, 7.13 mmol, 0.1 eq) and K₃PO₄ (30.27 g, 142.62 mmol, 2 eq) in turns at 25° C., the reaction mixture was heated to 90° C. and stirred at 90° C. for 12 h. 3 reactions were combined for work up. The reaction mixture was filtered and the filtrate was concentrated under reduced pressure to give a residue. The residue was diluted with H₂O (1500 mL) at 25° C. and extracted with DCM (500 mL×3).

The combined organic layers were washed with brine (100 mL×2), dried over Na₂SO₄, filtered and concentrated under reduced pressure to give a residue. The crude product was triturated with MTBE (800 mL) at 25° C. for 30 min to give the title compound (79.0 g, 148.49 mmol, 82% yield) as a yellow solid. ¹H NMR (CDCl₃, 400 MHz) δ 11.20 (s, 1H), 10.01 (s, 1H), 8.05-7.90 (m, 3H), 7.27-7.26 (m, 1H), 7.17 (d, J=8.4 Hz, 1H), 7.02 (d, J=8.8 Hz, 1H), 6.67 (d, J=8.4 Hz, 1H), 4.93-4.85 (m, 1H), 4.44 (d, J=6.4 Hz, 1H), 3.37-3.20 (m, 4H), 2.40 (s, 3H), 2.07 (s, 3H), 1.73-1.52 (m, 9H).

### Preparation of [4-[6-chloro-3-[1-[3,6-dimethyl-4-oxo-2-(1-piperidyl)chromen-8-yl]ethylamino]-2-pyridyl]-2-formyl-phenyl]trifluoromethanesulfonate

To a solution of 5-[6-chloro-3-[1-[3,6-dimethyl-4-oxo-2-(1-piperidyl)chromen-8-yl]ethylamino]-2-pyridyl]-2-hydroxy-benzaldehyde (35.00 g, 65.78 mmol, 1 eq), TEA (19.97 g, 197.35 mmol, 3 eq) and DMAP (804 mg, 6.58 mmol, 0.1 eq) in DCM (800 mL) was added [N-(trifluoromethyl sulfonyloxy)anilino]trifluoromethanesulfonate (25.85 g, 72.36 mmol, 1.1 eq) in portions at 0° C., the mixture was stirred at 25° C. for 2 h. 3 reactions were combined for work up. The reaction mixture was filtered and the filtrate was concentrated under reduced pressure. The residue was diluted with H₂O (1500 mL) and extracted with DCM (200 mL×3). The combined organic layers were washed with brine (50 mL×3), dried over Na₂SO₄, filtered and concentrated under reduced pressure to give a residue. The crude product was triturated with MTBE (800 mL) at 25° C. for 30 min to give the title compound (78 g, 117.45 mmol, 79.10% yield) as a yellow solid. ¹H NMR (CDCl₃, 400 MHz) δ 10.37 (s, 1H), 8.46 (d, J=2.4 Hz, 1H), 8.15 (d, J=6.4 Hz, 1H), 7.94 (s, 1H), 7.61 (d, J=8.8 Hz, 1H), 7.27-7.26 (m, 1H), 7.06 (d, J=8.4 Hz, 1H), 6.70 (d, J=8.4 Hz, 1H), 5.00-4.90 (m, 1H), 4.40 (d, J=3.2 Hz, 1H), 3.48-3.30 (m, 4H), 2.41 (s, 3H), 2.03 (s, 3H), 1.73-1.65 (m, 6H), 1.57 (s, 3H).

### Preparation of 5-(6-chloro-3-((1-(3,6-dimethyl-4-oxo-2-(piperidin-1-yl)-4H-chromen-8-yl)ethyl)amino)pyridin-2-yl)-2-(4,4,5,5-tetramethyl-1,3,2-dioxaborolan-2-yl)benzaldehyde

To a solution of [4-[6-chloro-3-[1-[3,6-dimethyl-4-oxo-2-(1-piperidyl)chromen-8-yl]ethylamino]-2-pyridyl]-2-formyl-phenyl]trifluoromethanesulfonate (23.00 g, 34.63 mmol, 1 eq) and B₂Pin₂ (35.18 g, 138.53 mmol, 4 eq) in dioxane (500 mL) were added Pd(PPh₃)₂Cl₂ (2.43 g, 3.46 mmol, 0.1 eq) and KOAc (10.20 g, 103.90 mmol, 3 eq) in turns at 25° C., the reaction mixture was stirred at 80° C. for 1 h. 3 reactions were combined for work up. The reaction mixture was filtered and the filtrate was concentrated under reduced pressure to give a residue. The crude product was purified by a silica gel column chromatography (PE:EtOAc=1:1-0:1) by 2 times to give the title compound (40.00 g, 62.31 mmol, 59.97% yield) as a yellow solid. ¹H NMR (CDCl₃400 MHz) δ 10.55 (s, 1H), 8.27 (s, 1H), 7.97-7.95 (m, 2H), 7.89-7.87 (m, 1H), 7.81 (s, 1H), 7.27-7.26 (m, 1H), 6.93 (d, J=8.4 Hz, 1H), 6.57 (d, J=8.8 Hz, 1H), 4.81-4.80 (m, 1H), 4.40-4.39 (m, 1H), 3.26-3.22 (m, 4H), 2.32 (s, 3H), 2.00 (s, 3H), 1.64-1.59 (m, 6H), 1.46-1.44 (m, 3H), 1.17 (s, 12H)

### Preparation of 8-[1-[[6-chloro-2-(1-hydroxy-2,3,1-benzoxazaborinin-6-yl)-3-pyridyl]amino]ethyl]-3,6-dimethyl-2-(1-piperidyl)chromen-4-one

To a solution of 5-[6-chloro-3-[1-[3,6-dimethyl-4-oxo-2-(1-piperidyl)chromen-8-yl]ethylamino]-2-pyridyl]-2-(4,4,5,5-tetramethyl-1,3,2-dioxaborolan-2-yl)benzaldehyde (10.00 g, 15.58 mmol, 1 eq) and NH₂OH·HCl (2.16 g, 31.15 mmol, 2 eq) in EtOH (100 mL)/H₂O (100 mL) was added KOAc (3.06 g, 31.15 mmol, 2 eq) in one portion at 25° C., the reaction mixture was heated to 45° C. for 16 h. 4 parallel reactions were combined for work up. The reaction mixture was cooled to 20° C. and filtered. The filter cake was collected and dried in vacuo to give the title compound (26.00 g, 46.69 mmol, 74.94% yield) as a white solid. ¹H NMR (DMSO-d₆, 400 MHz) δ 9.00 (s, 1H), 8.74 (s, 1H), 8.18 (d, J=7.6 Hz, 1H), 8.11-8.02 (m, 2H), 7.62 (s, 1H), 7.48 (s, 1H), 7.21 (d, J=8.8 Hz, 1H), 6.99 (d, J=8.8 Hz, 1H), 5.84 (d, J=7.6 Hz, 1H), 4.92 (t, J=6.8 Hz, 1H), 3.40-3.20 (m, 4H), 2.51 (s, 3H), 1.90 (s, 3H), 1.6-1.50 (m, 6H), 1.52 (d, J=6.4 Hz, 3H).

### Preparation of 8-[(1S)-1-[[6-chloro-2-(1-hydroxy-2,3,1-benzoxazaborinin-6-yl)-3-pyridyl]amino]ethyl]-3,6-dimethyl-2-(1-piperidyl)chromen-4-one and 8-[(1R)-1-[[6-chloro-2-(1-hydroxy-2,3,1-benzoxazaborinin-6-yl)-3-pyridyl]amino]ethyl]-3,6-dimethyl-2-(1-piperidyl)chromen-4-one

8-[1-[[6-chloro-2-(1-hydroxy-2,3,1-benzoxazaborinin-6-yl)-3-pyridyl]amino]ethyl]-3,6-dimeth yl-2-(1-piperidyl)chromen-4-one (29.0 g, 52.08 mmol, 1 eq) was purified by SFC (column: ChiralPak IH, 250×50 mm, 10 um; mobile phase: [CO2-IPA]; B %: 55%, isocratic elution mode) to give the title compound, Isomer 1 (11.9 g, 21.37 mmol, 41.03% yield) as a white solid and the title compound, Isomer 2 (12.5 g, 22.45 mmol, 43.10% yield) as a white solid. Isomer 1: ¹H NMR (DMSO-d₆, 400 MHz) δ 9.52 (s, 1H), 8.75 (s, 1H), 8.19 (d, J=7.6 Hz, 1H), 8.15-8.02 (m, 2H), 7.63 (s, 1H), 7.48 (s, 1H), 7.21 (d, J=8.8 Hz, 1H), 7.00 (d, J=8.8 Hz, 1H), 5.86 (d, J=7.2 Hz, 1H), 4.93 (t, J=6.8 Hz, 1H), 3.34-3.20 (m, 4H), 2.34 (s, 3H), 1.90 (s, 3H), 1.60-1.50 (m, 6H), 1.52 (d, J=6.4 Hz, 3H). MS (ESI): mass calcd. For C₃₀H₃₀BClN₄O₄ 556.20, m/z found 557.0 [M+H]⁺. HPLC: 99.28% (220 nm), 99.79% (254 nm). Chiral purity: 100% ee. Isomer 2: ¹H NMR (DMSO-d₆, 400 MHz) δ 9.52 (s, 1H), 8.75 (s, 1H), 8.19 (d, J=7.6 Hz, 1H), 8.15-8.02 (m, 2H), 7.63 (s, 1H), 7.48 (s, 1H), 7.21 (d, J=8.8 Hz, 1H), 7.00 (d, J=8.8 Hz, 1H), 5.86 (d, J=7.2 Hz, 1H), 4.93 (t, J=6.8 Hz, 1H), 3.34-3.20 (m, 4H), 2.34 (s, 3H), 1.90 (s, 3H), 1.60-1.50 (m, 6H), 1.52 (d, J=6.4 Hz, 3H). MS (ESI): mass calcd. For C₃₀H₃₀BClN₄O₄ 556.20, m/z found 557.1 [M+H]⁺. HPLC: 98.01% (220 nm), 98.15% (254 nm). Chiral purity: 98.36% ee.

### Example 62

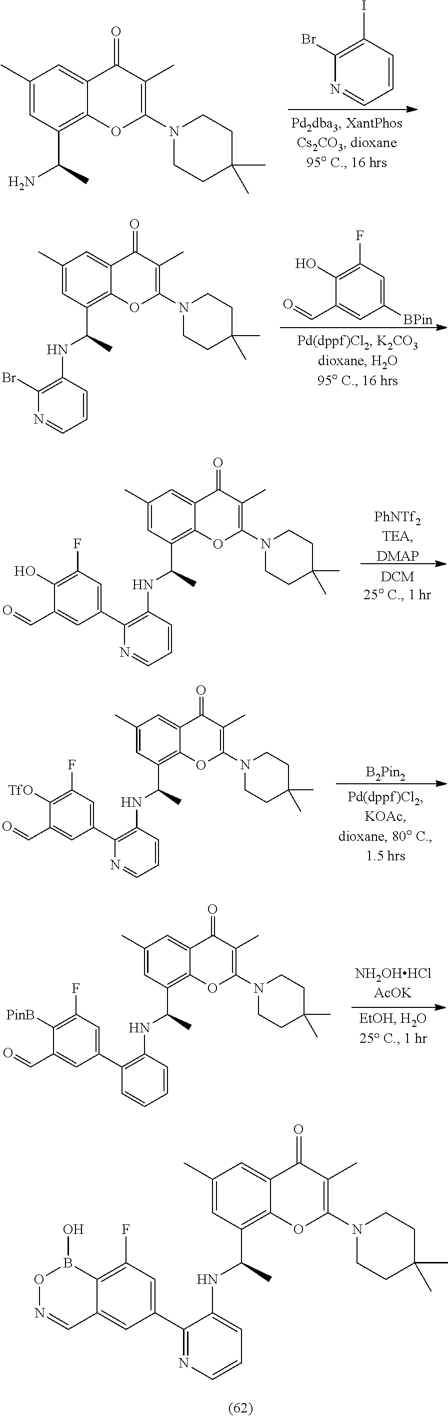

### Preparation of 8-[(1R)-1-[(2-bromo-3-pyridyl)amino]ethyl]-2-(4,4-dimethyl-1-piperidyl)-3,6-dimethyl-chromen-4-one

The title compound was prepared as yellow oil (2.50 g, 5.16 mmol, 89.21% yield) according to the similar procedure in Example 78, using 8-[(1R)-1-aminoethyl]-2-(4,4-dimethyl-1-piperidyl)-3,6-dimethyl-chromen-4-one (1.90 g, 5.78 mmol, 1 eq) and 2-bromo-3-iodo-pyridine (3.28 g, 11.57 mmol, 2 eq).

¹H NMR (DMSO-d₆, 400 MHz) δ 7.64 (d, J=1.2 Hz, 1H), 7.59 (dd, J=1.6, 4.4 Hz, 1H), 7.45 (d, J=2.0 Hz, 1H), 7.09 (dd, J=4.8, 8.0 Hz, 1H), 6.76 (dd, J=1.2, 8.0 Hz, 1H), 5.54 (d, J=6.8 Hz, 1H), 5.02 (q, J=6.8 Hz, 1H), 3.44-3.39 (m, 4H), 2.31 (s, 3H), 1.91 (s, 3H), 1.63 (d, J=6.8 Hz, 3H), 1.47-1.44 (m, 4H), 0.98 (s, 6H).

### Preparation of 5-[3-[[(1R)-1-[2-(4,4-dimethyl-1-piperidyl)-3,6-dimethyl-4-oxo-chromen-8-yl]ethyl]amino]-2-pyridyl]-3-fluoro-2-hydroxy-benzaldehyde

The title compound was prepared as a yellow solid (0.90 g, 1.66 mmol, 53.47% yield) according to the procedures described herein and the synthetic scheme, using 8-[(1R)-1-[(2-bromo-3-pyridyl)amino]ethyl]-2-(4,4-dimethyl-1-piperidyl)-3,6-dimethyl-chromen-4-one (1.50 g, 3.10 mmol, 1 eq) and 3-fluoro-2-hydroxy-5-(4,4,5,5-tetramethyl-1,3,2-dioxaborolan-2-yl)benzaldehyde (1.65 g, 6.19 mmol, 2 eq). ¹H NMR (DMSO-d₆, 400 MHz) δ 11.11 (s, 1H), 10.33 (s, 1H), 7.93-7.85 (m, 3H), 7.62 (d, J=1.6 Hz, 1H), 7.50 (s, 1H), 7.05 (dd, J=4.8, 8.4 Hz, 1H), 6.84 (d, J=7.6 Hz, 1H), 5.62 (d, J=7.2 Hz, 1H), 4.96 (q, J=6.8 Hz, 1H), 3.40-3.35 (m, 4H), 2.32 (s, 3H), 1.92 (s, 3H), 1.52 (d, J=6.8 Hz, 3H), 1.45-1.43 (m, 4H), 0.98 (s, 6H).

### Preparation of [4-[3-[[(1R)-1-[2-(4,4-dimethyl-1-piperidyl)-3,6-dimethyl-4-oxo-chromen-8-yl]ethyl]amino]-2-pyridyl]-2-fluoro-6-formyl-phenyl]trifluoromethanesulfonate

The title compound was prepared as a yellow solid (1.10 g, 1.63 mmol, 98.33% yield) according to the procedures described herein and the synthetic scheme, using 5-[3-[[(1R)-1-[2-(4,4-dimethyl-1-piperidyl)-3,6-dimethyl-4-oxo-chromen-8-yl]ethyl]amino]-2-pyridyl]-3-fluoro-2-hydroxy-benzaldehyde (0.90 g, 1.66 mmol, 1 eq) and PhNTf₂ (1.18 g, 3.31 mmol, 2 eq). ¹H NMR (DMSO-d₆, 400 MHz) δ 10.13 (s, 1H), 8.38 (s, 1H), 8.31 (dd, J=2.0, 11.2 Hz, 1H), 7.96 (d, J=4.4 Hz, 1H), 7.62 (d, J=1.2 Hz, 1H), 7.48 (d, J=1.6 Hz, 1H), 7.13 (dd, J=4.4, 8.4 Hz, 1H), 6.85 (d, J=8.0 Hz, 1H), 6.11 (d, J=7.2 Hz, 1H), 4.98 (q, J=6.8 Hz, 1H), 3.40-3.38 (m, 4H), 2.31 (s, 3H), 1.92 (s, 3H), 1.54 (d, J=6.8 Hz, 3H), 1.47-1.44 (m, 4H), 0.98 (s, 6H).

### Preparation of 5-[3-[[(1R)-1-[2-(4,4-dimethyl-1-piperidyl)-3,6-dimethyl-4-oxo-chromen-8-yl]ethyl]amino]-2-pyridyl]-3-fluoro-2-(4,4,5,5-tetramethyl-1,3,2-dioxaborolan-2-yl)benzaldehyde

The title compound was prepared as a yellow solid (0.7 g, 1.07 mmol, 72.37% yield) according to the procedures described herein and the synthetic scheme, using [4-[3-[[(1R)-1-[2-(4,4-dimethyl-1-piperidyl)-3,6-dimethyl-4-oxo-chromen-8-yl]ethyl]amino]-2-pyridyl]-2-fluoro-6-formyl-phenyl]trifluoromethanesulfonate (1.00 g, 1.48 mmol, 1 eq), and B₂Pin₂ (1.13 g, 4.44 mmol, 3 eq). ¹H NMR (DMSO-d₆, 400 MHz) δ 10.01 (s, 1H), 8.20 (s, 1H), 7.95 (d, J=3.2 Hz, 1H), 7.82 (d, J=8.8 Hz, 1H), 7.62 (s, 1H), 7.50 (d, J=2.0 Hz, 1H), 7.12-7.09 (m, 1H), 6.91 (d, J=8.0 Hz, 1H), 5.56-5.55 (m, 1H), 4.95 (q, J=7.2 Hz, 1H), 3.34-3.30 (m, 4H), 2.32 (s, 3H), 1.90 (s, 3H), 1.52 (d, J=6.8 Hz, 3H), 1.48-1.38 (m, 16H), 1.07 (s, 6H).

### Preparation of 2-(4,4-dimethyl-1-piperidyl)-8-[(1R)-1-[[2-(8-fluoro-1-hydroxy-2,3,1-benzoxazaborinin-6-yl)-3-pyridyl]amino]ethyl]-3,6-dimethyl-chromen-4-one

The title compound was prepared as a white solid (203.8 mg, 354.59 μmol, 35.65% yield) according to the procedures described herein and the synthetic scheme, using 5-[3-[[(1R)-1-[2-(4,4-dimethyl-1-piperidyl)-3,6-dimethyl-4-oxo-chromen-8-yl]ethyl]amino]-2-pyridyl]-3-fluoro-2-(4,4,5,5-tetramethyl-1,3,2-dioxaborolan-2-yl) benzaldehyde (650 mg, 994.51 μmol, 1 eq) and hydroxylamine; hydrochloride (104 mg, 1.49 mmol, 1.5 eq). ¹H NMR (DMSO-d₆, 400 MHz) δ 8.81 (br s, 1H), 8.67 (s, 1H), 7.95 (d, J=3.6 Hz, 1H), 7.91 (s, 1H), 7.76 (d, J=10.0 Hz, 1H), 7.62 (d, J=1.2 Hz, 1H), 7.50 (s, 1H), 7.10 (dd, J=3.6, 8.0 Hz, 1H), 6.89 (d, J=8.0 Hz, 1H), 5.76 (d, J=6.8 Hz, 1H), 4.95 (q, J=7.2 Hz, 1H), 3.34-3.33 (m, 4H), 2.32 (s, 3H), 1.90 (s, 3H), 1.52 (d, J=6.4 Hz, 3H), 1.43-1.40 (m, 4H), 0.96 (s, 6H). MS (ESI): mass calcd. For C₃₂H₃₄BFN₄O₄ 568.27, m/z found 569.3 [M+H]⁺. HPLC: 98.90% (220 nm), 99.23% (254 nm). Chiral purity: 99.78% ee.

### Example 63

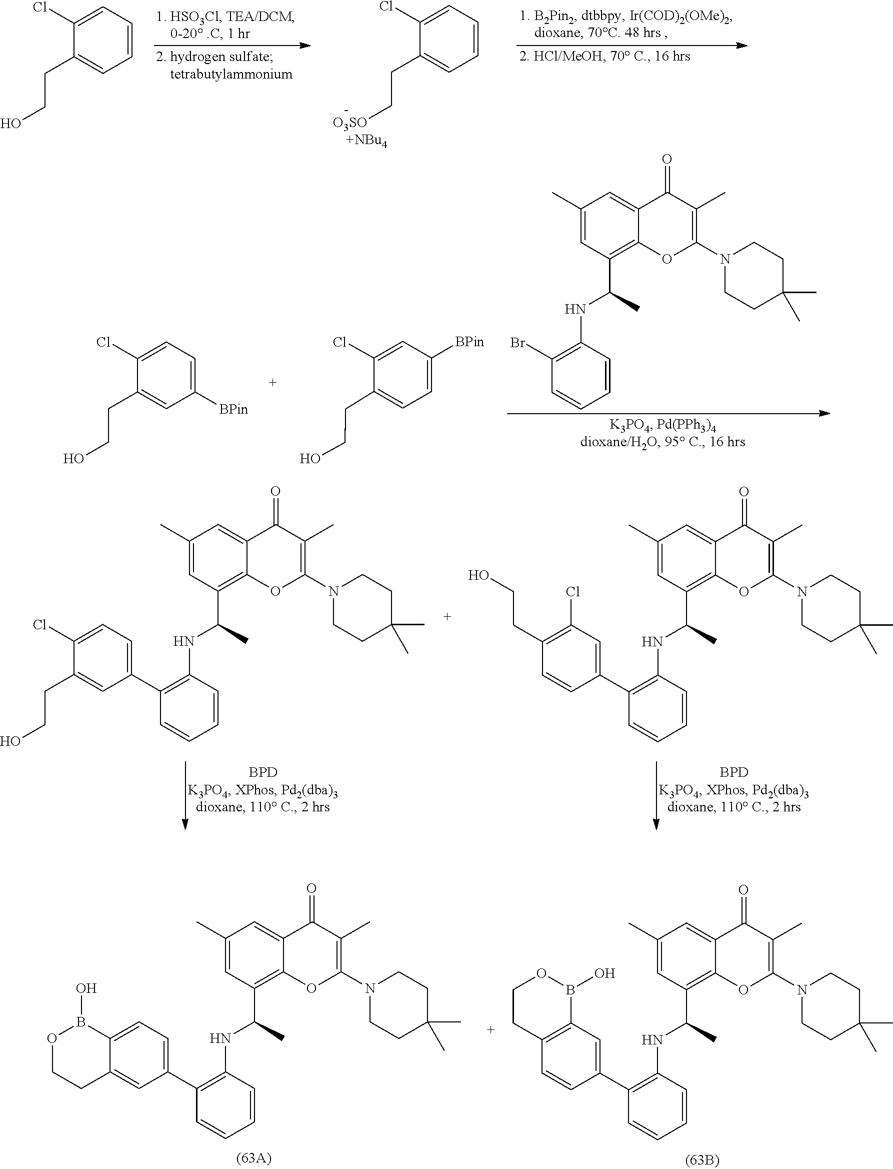

### Preparation of 2-(2-chlorophenyl)ethyl sulfate;tetrabutylammonium

To a solution of 2-(2-chlorophenyl)ethanol (5.00 g, 31.93 mmol, 1 eq) and TEA (12.92 g, 127.71 mmol, 17.78 mL, 4 eq) in DCM (50 mL) was added sulfurochloridic acid (3.72 g, 31.93 mmol, 2.12 mL, 1 eq) at 0° C. under N₂, the resulting reaction mixture was allowed to warm to 25° C. and stirred at 25° C. for 1 h. The reaction mixture quenched by addition 10% aq.NaOH (50 mL) and extracted with DCM (80 mL×3). To the aqueous layer was added hydrogen sulfate;tetrabutylammonium (8.67 g, 25.54 mmol, 0.8 eq) and then extracted with DCM (50 mL×3) again. The combined organic layers were washed with H₂O (50 mL×3), dried over Na₂SO₄, filtered and concentrated under reduced pressure to give the title compound (11.00 g, 23.01 mmol, 72.06% yield) as a white solid. ¹H NMR (CDCl₃, 400 MHz) δ 7.37 (d, J=1.6 Hz, 1H), 7.29-7.27 (m, 1H), 7.16-7.11 (m, 2H), 4.25 (t, J=7.2 Hz, 2H), 3.26-3.22 (m, 8H), 3.14 (t, J=7.2 Hz, 2H), 1.66-1.60 (m, 8H), 1.44-1.39 (m, 8H), 0.98 (t, J=7.2 Hz, 12H).

### Preparation of 2-(2-chloro-5-(4,4,5,5-tetramethyl-1,3,2-dioxaborolan-2-yl)phenyl) ethan-1-ol and 2-(2-chloro-4-(4,4,5,5-tetramethyl-1,3,2-dioxaborolan-2-yl)phenyl)ethan-1-ol

To a mixture of 2-(2-chlorophenyl)ethyl sulfate;tetrabutylammonium (3.00 g, 6.27 mmol, 1 eq) and B₂Pin₂ (2.39 g, 9.41 mmol, 1.5 eq) in dioxane (30 mL) was added Ir(COD)₂(OMe)₂ (416 mg, 627.45 gmol, 0.1 eq) at 25° C., the reaction mixture was bubbled with N₂ for 2 min. Then 4-tert-butyl-2-(4-tert-butyl-2-pyridyl)pyridine (337 mg, 1.25 mmol, 0.2 eq) was added in oen portion under N₂ in glovebox. The resulting mixture was heated to 70° C. and stirred at 70° C. for 48 h under N₂ atmosphere. Two parallel reaction mixtures were combined, concentrated under reduced pressure and dissolved in HCl (1 M in MeOH, 60 mL, 9.56 eq). Then the reaction mixture was heated to 50° C. and stirred at 50° C. for 16 h. The reaction mixture was cooled to 25° C., quenched by adding water (50 mL) and extracted with EtOAc (40 mL×3). The combined organic layers were washed brine (50 mL), dried over Na₂SO₄, filtered and concentrated under reduced pressure. The residue was purified by flash silica gel chromatography (Biotage®; 120 g SepaFlash® Silica Flash Column, Eluent of 0~40% Ethyl acetate/Petroleum ether gradient @150 mL/min) to give Part 1: a mixture of [4-chloro-3-(2-hydroxyethyl)phenyl]boronic acid and [3-chloro-4-(2-hydroxyethyl)phenyl]boronic acid (1.67 g, 8.33 mmol, 66.40% yield) as colorless oil. Part 2: a mixture of 2-(2-chloro-5-(4,4,5,5-tetramethyl-1,3,2-dioxaborolan-2-yl)phenyl)ethan-1-ol and 2-(2-chloro-4-(4,4,5,5-tetramethyl-1,3,2-dioxaborolan-2-yl)phenyl) ethan-1-ol (0.6 g, 2.12 mmol, 16.92% yield) as light yellow oil. ¹H NMR (DMSO-d₆, 400 MHz) δ 7.80 (s, 0.5H), 7.70 (d, J=1.2 Hz, 0.5H), 7.63-7.59 (m, 1H), 7.36 (d, J=8.0 Hz, 0.5H), 7.28 (d, J=6.4 Hz, 0.5H), 3.90-3.86 (m, 2H), 3.04 (t, J=6.8 Hz, 2H), 1.34 (s, 12H).

### Preparation of 8-[(1R)-1-[2-[4-chloro-3-(2-hydroxyethyl)phenyl]anilino]ethyl]-2-(4,4-dimethyl-1-piperidyl)-3,6-dimethyl-chromen-4-one and 8-[(1R)-1-[2-[3-chloro-4-(2-hydroxyethyl)phenyl]anilino]ethyl]-2-(4,4-dimethyl-1-piperidyl)-3,6-dimethyl-chromen-4-one

To a mixture of 2-[2-chloro-5-(4,4,5,5-tetramethyl-1,3,2-dioxaborolan-2-yl)phenyl]ethanol and 2-[2-chloro-4-(4,4,5,5-tetramethyl-1,3,2-dioxaborolan-2-yl)phenyl]ethanol (262 mg, 930 μmol, 0.75 eq), 8-[(1R)-1-(2-bromoanilino)ethyl]-2-(4,4-dimethyl-1-piperidyl)-3,6-dimethyl-chromen-4-one (300 mg, 620.55 μmol, 1 eq) and K₃PO₄ (263 mg, 1.24 mmol, 2 eq) in dioxane (5 mL)/H₂O (1 mL) was added Pd(PPh₃)₄ (72 mg, 62.06 μmol, 0.1 eq) under N₂. Then the reaction mixture was degassed and purged with N₂ for 3 times, heated to 95° C. and stirred at 95° C. for 16 h under N₂ atmosphere. The reaction mixture was cooled to 20° C., filtered and the filtrate was concentrated under reduced pressure. The residue was purified by prep-HPLC (column: Waters Xbridge Prep OBD C18 150 ×40 mm×10 um; mobile phase: [H₂O (10 mM NH₄HCO₃)-AC□]; gradient:70%-95% B over 8.0 min) to give the title compound, Isomer 1 (0.15 g, 255.10 μmol, 41.11% yield) as a white solid and the title compound, Isomer 2 (0.3 g, 536.54 μmol, 86.46% yield) as a white solid. Isomer 1: ¹H NMR (DMSO-d₆, 400 MHz) δ 7.62 (s, 1H), 7.50 (d, J=6.0 Hz, 1H), 7.49-7.46 (m, 2H), 7.33 (d, J=2.0 Hz, 1H), 7.04-7.02 (m, 2H), 6.68 (t, J=7.2 Hz, 1H), 6.47 (d, J=8.4 Hz, 1H), 4.99-4.93 (m, 2H), 4.75 (t, J=5.2 Hz, 1H), 3.70-3.66 (m, 2H), 3.40-3.34 (m, 4H), 2.89 (t, J=6.8 Hz, 2H), 2.51 (s, 3H), 1.92 (s, 3H), 1.48 (d, J=6.0 Hz, 3H), 1.44 (s, 4H), 0.99 (s, 6H). MS (ESI): mass calcd. For C₃₄H₃₉ClN₂O₃ 558.26, m/z found 559.2 [M+H]⁺. HPLC: 95.09% (220 nm), 95.56% (254 nm). Isomer 2: ¹H NMR (DMSO-d₆, 400 MHz) δ 7.61 (s, 1H), 7.46-7.44 (m, 3H), 7.43-7.39 (m, 1H), 7.04 (t, J=7.2 Hz, 1H), 6.99 (d, J=1.2 Hz, 1H), 6.68 (t, J=7.6 Hz, 1H), 6.49 (t, J=8.4 Hz, 1H), 4.95 (t, J=6.8 Hz, 1H), 4.91 (t, J=6.8 Hz, 1H), 3.69-3.64 (m, 2H), 3.36-3.33 (m, 4H), 2.89 (t, J=6.8 Hz, 2H), 2.32 (s, 3H), 1.91 (s, 3H), 1.47 (d, J=6.4 Hz, 3H), 1.42 (t, J=5.2 Hz, 4H), 0.97 (s, 6H). MS (ESI): mass calcd. For C₃₄H₃₉ClN₂O₃ 558.26, m/z found 559.1 [M+H]⁺. HPLC: 100.00% (220 nm), 100.00% (254 nm).

### Preparation of 2-(4,4-dimethyl-1-piperidyl)-8-[(1R)-1-[2-(1-hydroxy-3,4-dihydro-2,1-benzoxaborinin-6-yl)anilino]ethyl]-3,6-dimethyl-chromen-4-one

To a mixture of 8-[(1R)-1-[2-[4-chloro-3-(2-hydroxyethyl)phenyl]anilino]ethyl]-2-(4,4-dimethyl-1-piperidyl)-3,6-dimethyl-chromen-4-one (140 mg, 250.39 μmol, 1 eq), B₂Pin₂ (318 mg, 1.25 mmol, 5 eq), KOAc (74 mg, 751.16 μmol, 3 eq), and XPhos (12 mg, 25.04 μmol, 0.1 eq) in dioxane (3 mL) was added Pd₂(dba)₃ (23 mg, 25.04 μmol, 0.1 eq) under N₂. Then the reaction mixture was bubbled with N₂ for 2 min, heated to 110° C. and stirred at 110° C. for 2 h. The reaction mixture was cooled to 20° C., filtered and the filtrate was concentrated under reduced pressure. The residue was purified by prep-HPLC (column: Welch Xtimate C18 150×25 mm×5 um; mobile phase: [H₂O (10 mM NH₄HCO₃)-MeOH]; gradient:50%-90% B over 15.0 min). The residual aqueous solution was lyophilized to give the title compound (10.4 mg, 18.89 μmol, 7.55% yield) as a white solid. ¹H NMR (DMSO-d₆, 400 MHz) δ 8.46 (s, 1H), 7.77 (d, J=7.2 Hz, 1H), 7.62 (s, 1H), 7.45 (s, 1H), 7.33 (d, J=7.6 Hz, 1H), 7.25 (s, 1H), 7.03 (t, J=8.0 Hz, 1H), 7.00 (d, J=6.8 Hz, 1H), 6.68 (t, J=8.4 Hz, 1H), 6.54 (d, J=8.0 Hz, 1H), 4.99-4.93 (m, 1H), 4.80 (d, J=7.2 Hz, 1H), 4.10 (t, J=6.0 Hz, 2H), 3.26-3.23 (m, 4H), 2.88 (t, J=6.0 Hz, 2H), 2.32 (s, 3H), 1.91 (s, 3H), 1.48 (d, J=6.8 Hz, 3H), 1.40 (s, 4H), 0.96 (s, 6H). MS (ESI): mass calcd. For C₃₄H₃₉BN₂O₄ 550.3, m/z found 551.2 [M+H]⁺. HPLC: 100.00% (220 nm), 100.00% (254 nm). Chiral purity: 95.14% ee.

### Preparation of 2-(4,4-dimethyl-1-piperidyl)-8-[(1R)-1-[2-(1-hydroxy-3,4-dihydro-2,1-benzoxaborinin-7-yl)anilino]ethyl]-3,6-dimethyl-chromen-4-one

To a mixture of 8-[(1R)-1-[2-[3-chloro-4-(2-hydroxyethyl)phenyl]anilino]ethyl]-2-(4,4-dimethyl-1-piperidyl)-3,6-dimethyl-chromen-4-one (260 mg, 465.00 μmol, 1 eq), B₂Pin₂ (590 mg, 2.33 mmol, 5 eq), KOAc (137 mg, 1.40 mmol, 3 eq), and XPhos (22 mg, 46.50 gmol, 0.1 eq) in dioxane (5 mL) was added Pd₂(dba)₃ (43 mg, 46.50 μmol, 0.1 eq) under N₂. Then the reaction mixture was bubbled with N₂ for 2 mins, heated to 110° C. and stirred at 110° C. for 2 h. The reaction mixture was cooled to 20° C., filtered and the filtrate was concentrated under reduced pressure. The residue was purified by prep-HPLC (column: Waters Xbridge Prep OBD C18 150×40 mm×10 um; mobile phase: [H₂O (10 mM NH₄HCO₃)-AC□]; gradient: 55%-85% B over 8.0 min). The residual aqueous solution was lyophilized to give the title compound (91.8 mg, 165.96 μmol, 35.69% yield) as a white solid. ¹H NMR (DMSO-d₆, 400 MHz) δ 11.01 (s, 1H), 8.43 (br s, 1H), 7.86 (d, J=2.0 Hz, 1H), 7.57-7.50 (m, 2H), 7.45 (dd, J=2.4, 8.8 Hz, 1H), 7.23 (d, J=2.0 Hz, 1H), 7.13-7.06 (m, 1H), 6.64 (d, J=8.8 Hz, 1H), 3.39 (t, J=6.8 Hz, 2H), 2.97 (t, J=7.2 Hz, 2H). MS (ESI): mass calcd. For C₃₄H_{39B}N₂O₄ 550.3, m/z found 551.2 [M+H]⁺. HPLC: 98.10% (220 nm), 98.67% (254 nm). Chiral purity: 96.46% ee.

### Example 64

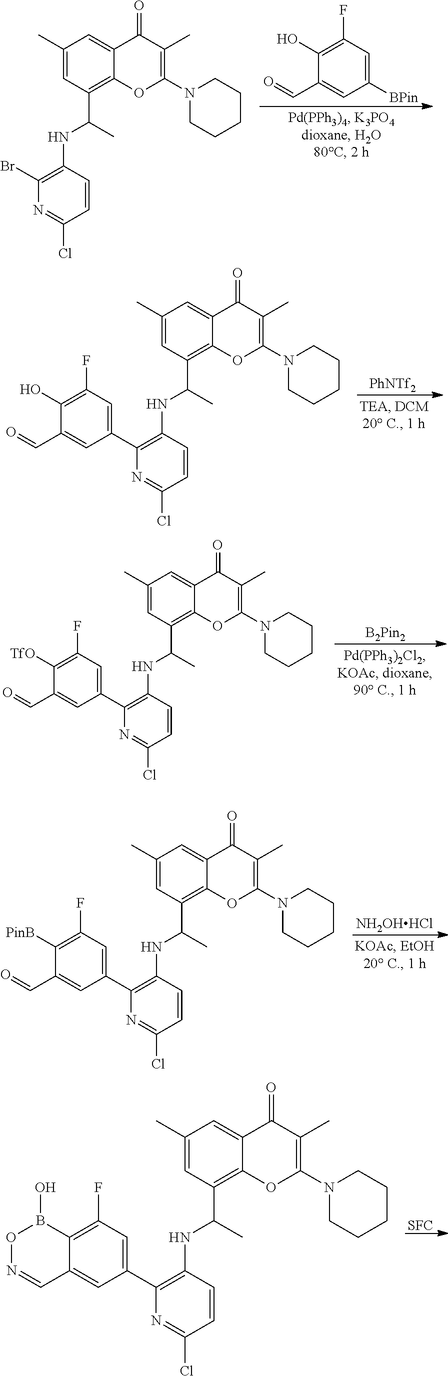

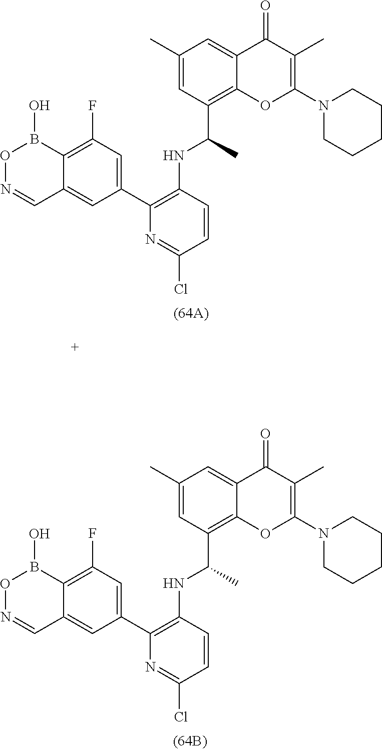 [SMILES: Cc1cc([C@@H](C)Nc2ccc(Cl)nc2-c2cc(F)c3c(c2)C=NOB3O)c2oc(N3CCCCC3)c(C)c(=O)c2c1 ; Cc1cc([C@H](C)Nc2ccc(Cl)nc2-c2cc(F)c3c(c2)C=NOB3O)c2oc(N3CCCCC3)c(C)c(=O)c2c1]

### Preparation of 5-[6-chloro-3-[1-[3,6-dimethyl-4-oxo-2-(1-piperidyl)chromen-8-yl]ethylamino]-2-pyridyl]-3-fluoro-2-hydroxy-benzaldehyde

A mixture of 8-[1-[(2-bromo-6-chloro-3-pyridyl)amino]ethyl]-3,6-dimethyl-2-(1-piperidyl) chromen-4-one (2.20 g, 4.48 mmol, 1 eq), 3-fluoro-2-hydroxy-5-(4,4,5,5-tetramethyl-1,3,2-dioxaborolan-2-yl)benzaldehyde (1.43 g, 5.38 mmol, 1.2 eq), Pd(PPh₃)₄ (259 mg, 224 μmol, 0.05 eq) and K₃PO₄ (1.90 g, 8.96 mmol, 2 eq) in dioxane (20 mL)/H₂O (4 mL) was de-gassed and then heated to 80° C. for 2 h under N₂. The reaction mixture was filtered and the filtrate was concentrated in vacuo. The residue was purified by flash silica gel chromatography (ISCO®; 40 g SepaFlash® Silica Flash Column, Eluent of 0~40% Ethyl acetate/Petroleum ether gradient @100 mL/min) to give the title compound (1.70 g, 3.09 mmol, 68.96% yield) as yellow solid, which was used directly for next step.

### Preparation of 4-(6-chloro-3-((1-(3,6-dimethyl-4-oxo-2-(piperidin-1-yl)-4H-chromen-8-yl)ethyl)amino)pyridin-2-yl)-2-fluoro-6-formylphenyltrifluoromethanesulfonate

To a solution of 5-[6-chloro-3-[1-[3,6-dimethyl-4-oxo-2-(1-piperidyl)chromen-8-yl]ethylamino]-2-pyridyl]-3-fluoro-2-hydroxy-benzaldehyde (1.50 g, 2.73 mmol, 1 eq) and 1,1,1-trifluoro-N-phenyl-N-(trifluoromethylsulfonyl)methanesulfonamide (1.95 g, 5.45 mmol, 2 eq) in DCM (30 mL) was added TEA (552 mg, 5.45 mmol, 759 μL, 2 eq) and DMAP (34 mg, 273 μmol, 0.1 eq) in one portion at 20° C., the reaction mixture was stirred at 20° C. for 1 h. The reaction mixture was concentrated in vacuo and the residue was purified by flash silica gel chromatography (ISCO®; 40 g SepaFlash® Silica Flash Column, Eluent of 0~40% Ethyl acetate/Petroleum ether gradient @80 mL/min) to give the title compound (1.80 g, 2.64 mmol, 96.77% yield) as yellow solid. ¹H NMR (CDCl₃, 400 MHz) δ 10.28 (s, 1H), 8.26 (d, J=2.0 Hz, 1H), 8.01 (d, J=12.0 Hz, 1H), 7.90 (s, 1H), 7.29-7.29 (m, 1H), 7.06 (d, J=8.4 Hz, 1H), 6.71 (d, J=8.8 Hz, 1H), 4.95-4.94 (m, 1H), 4.47-4.46 (m. 1H), 3.37-3.35 (m, 4H), 2.38 (s, 3H), 2.06 (s, 3H), 1.71 (s, 6H), 1.62 (d, J=6.4 Hz, 3H).

### Preparation of 5-(6-chloro-3-((1-(3,6-dimethyl-4-oxo-2-(piperidin-1-yl)-4H-chromen-8-yl)ethyl)amino)pyridin-2-yl)-3-fluoro-2-(4,4,5,5-tetramethyl-1,3,2-dioxaborolan-2-yl)benzaldehyde

To a solution of 4-(6-chloro-3-((1-(3,6-dimethyl-4-oxo-2-(piperidin-1-yl)-4H-chromen-8-yl) ethyl)amino)pyridin-2-yl)-2-fluoro-6-formylphenyltrifluoromethanesulfonate (1.10 g, 1.61 mmol, 1 eq) and B₂Pin₂ (1.64 g, 6.45 mmol, 4 eq) in dioxane (20 mL) was added KOAc (475 mg, 4.84 mmol, 3 eq) and Pd(PPh₃)₂Cl₂ (113 mg, 161 μmol, 0.1 eq) in turns at 20° C. The mixture was heated to 90° C. and stirred at 90° C. for 1 h. The reaction mixture was cooled to 20° C. and filtered through a pad of Celite. The filtrate was concentrated under reduced pressure to give the title compound (1.40 g, crude) as a yellow solid, which was used directly for next step without further purification. ¹H NMR (CDCl₃, 400 MHz) δ 10.10 (s, 1H), 8.03 (s, 1H), 7.89 (s, 1H), 7.69 (d, J=7.6 Hz, 1H), 7.24 (d, J=2.0 Hz, 1H), 7.03 (d, J=8.8 Hz, 1H), 6.76 (d, J=8.4 Hz, 1H), 4.90-4.86 (m, 1H), 4.42-4.41 (m, 1H), 3.33-3.32 (m, 4H), 2.37 (s, 3H), 2.05 (s, 3H), 1.69 (s, 6H), 1.55 (d, J=6.4 Hz, 3H), 1.19 (s, 12H).

### Preparation of 8-(1-((6-chloro-2-(8-fluoro-1-hydroxy-JH-benzo[d][1,2,6]oxazaborinin-6-yl)pyridin-3-yl)amino)ethyl)-3,6-dimethyl-2-(piperidin-1-yl)-4H-chromen-4-one

To a solution of 5-(6-chloro-3-((1-(3,6-dimethyl-4-oxo-2-(piperidin-1-yl)-4H-chromen-8-yl) ethyl)amino)pyridin-2-yl)-3-fluoro-2-(4,4,5,5-tetramethyl-1,3,2-dioxaborolan-2-yl)benzaldehyde (1.30 g, 1.97 mmol, 1 eq) in EtOH (20 mL) was added KOAc (387 mg, 3.94 mmol, 2 eq) and hydroxylamine;hydrochloride (274 mg, 3.94 mmol, 2 eq) in turns at 20° C., the reaction mixture was stirred at 20° C. for 1 h. The reaction mixture was concentrated in vacuo and the residue was triturated with EtOH (5 mL)/H₂O (15 mL) at 20° C. for 10 min. The suspension was filtered and the filter cake was directly purified by prep-HPLC (column: Agela DuraShell C18 250×70 mm×10 um; mobile phase: [H₂O (10 mM NH₄HCO₃)-AC□]; gradient: 25%-50% B over 17.0 min) to give the title compound (400 mg, 696 mol, 35.33% yield) as a white solid. ¹H NMR (CDCl₃, 400 MHz) δ 9.38 (brs, 1H), 8.76 (d, J=2.4 Hz, 1H), 7.91 (s, 1H), 7.79 (d, J=10.0 Hz, 1H), 7.61 (d, J=1.2 Hz, 1H), 7.45 (d, J=2.0 Hz, 1H), 7.21 (d, J=8.8 Hz, 1H), 6.97 (d, J=8.8 Hz, 1H), 6.02-6.00 (m, 1H), 4.95-4.88 (m, 1H), 3.28-3.27 (m. 4H), 2.31 (s, 3H), 1.89 (s, 3H), 1.59 (s, 6H), 1.51 (d, J=6.8 Hz, 3H).

### Preparation of (R)-8-(1-((6-chloro-2-(8-fluoro-1-hydroxy-1H-benzo[d][1,2,6]oxazaborinin-6-yl)pyridin-3-yl)amino)ethyl)-3,6-dimethyl-2-(piperidin-1-yl)-4H-chromen-4-one and (S)-8-(1-((6-chloro-2-(8-fluoro-1-hydroxy-JH-benzo[d][1,2,6]oxazaborinin-6-yl)pyridin-3-yl)amino)ethyl)-3,6-dimethyl-2-(piperidin-1-yl)-4H-chromen-4-one

8-[1-[[6-chloro-2-(8-fluoro-1-hydroxy-2,3,1-benzoxazaborinin-6-yl)-3-pyridyl]amino]ethyl]-3,6-dimethyl-2-(1-piperidyl)chromen-4-one (400 mg, 696 μmol, 1 eq) was purified by SFC (column: DAICEL CHIRALPAK IK (250 mm×25 mm, 10 um); mobile phase: [CO2-EtOH]; B %: 65%, isocratic elution mode) to give the title compound, Isomer 1 (136.3 mg, 231.80 μmol, 83.28% yield) as a white solid and the title compound, Isomer 2 (166.9 mg, 282.42 μmol, 45.10% yield) as a white solid.

Isomer 1: ¹H NMR (DMSO-d₆, 400 MHz) δ 8.07-7.86 (m, 1H), 7.62 (s, 1H), 7.44-7.33 (m, 3H), 7.14-7.04 (m, 1H), 6.88-6.86 (m, 1H), 5.70-5.44 (m, 1H), 4.96-4.92 (m, 1H), 3.22-3.20 (m, 4H), 2.31-2.25 (m, 3H), 1.90 (s, 3H), 1.60 (s, 6H), 1.52-1.51 (m, 3H). MS (ESI): mass calcd. For C₃₀H₂₉BClFN₄O₄ 574.84, m/z found 575.3 [M+H]⁺. HPLC: 97.76% (220 nm), 98.34% (254 nm). Chiral purity: 100% ee. Isomer 2: ¹H NMR (DMSO-d₆, 400 MHz) δ 7.99-7.87 (m, 1H), 7.62 (s, 1H), 7.44-7.43 (m, 1H), 7.30-7.28 (m, 2H), 7.13-7.11 (m, 1H), 6.85-6.79 (m, 1H), 5.45-5.32 (m, 1H), 4.99-4.92 (m, 1H), 3.20-3.14 (m, 4H), 2.31-2.23 (m, 3H), 1.90 (s, 3H), 1.60 (s, 6H), 1.52-1.51 (m, 3H). MS (ESI): mass calcd. For C₃₀H₂₉BClFN₄O₄ 574.84, m/z found 575.1 [M+H]⁺. HPLC: 97.27% (220 nm), 97.98% (254 nm). Chiral purity: 96.72% ee.

### Example 65

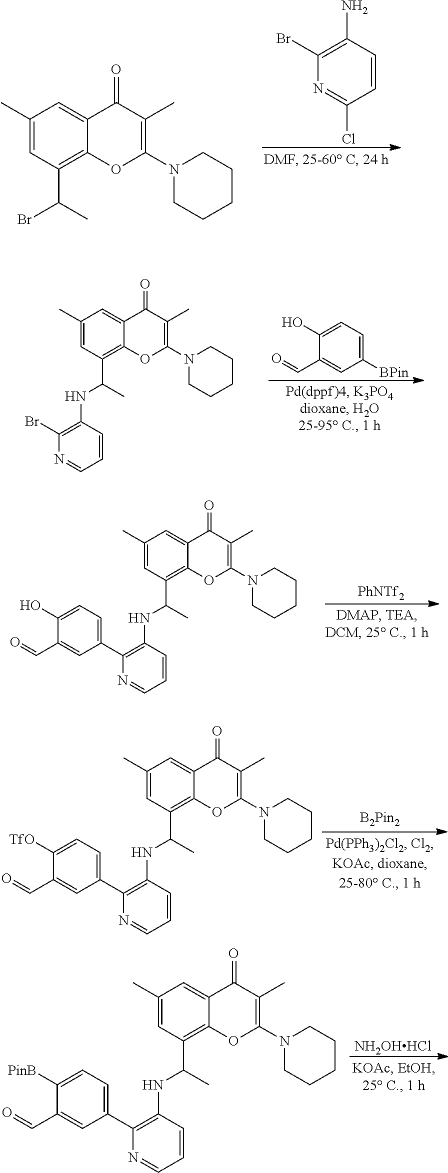

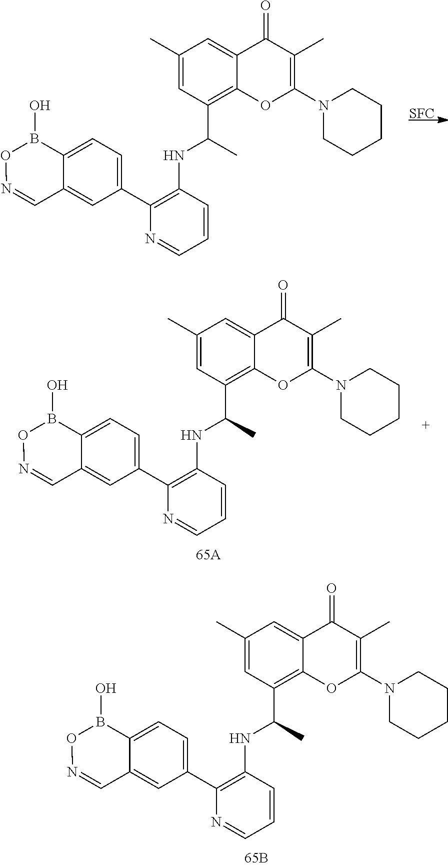

### Preparation of 8-(1-((2-bromopyridin-3-yl)amino)ethyl)-3,6-dimethyl-2-(piperidin-1-yl)-4H-chromen-4-one

To a solution of 8-(1-bromoethyl)-3,6-dimethyl-2-(1-piperidyl)chromen-4-one (3.00 g, 8.24 mmol, 1 eq) in DMF (30 mL) was added 2-bromopyridin-3-amine (2.28 g, 13.18 mmol, 1.6 eq) at 25° C., the reaction mixture was heated to 60° C. and stirred at 60° C. for 24 h. The reaction mixture was cooled to 0° C. and quenched with H₂O (60 mL) at 0° C. The resulting suspension was filtered and the filter cake was washed with PE (10 mL). The filter cake was directly purified by prep-HPLC (column: Welch Xtimate C18 180×70 mm×10 um; mobile phase: [H₂O (10 mM NH₄HCO₃)-AC□]; gradient: 50%-80% B over 20.0 min) to give the title compound (1.60 g, 3.51 mmol, 42.57% yield) as a white solid. ¹H NMR (DMSO-d₆, 400 MHz) δ 7.64 (s, 1H), 7.58 (d, J=4.8 Hz, 1H), 7.44 (s, 1H), 7.10-7.07 (m, 1H), 6.74 (d, J=6.8 Hz, 1H), 5.56 (d, J=7.2 Hz, 1H), 5.02 (t, J=6.8 Hz, 1H), 3.40-3.41 (m, 4H), 2.31 (s, 3H), 1.92 (s, 3H), 1.66-1.64 (m, 6H), 1.63 (s, 3H).

### Preparation of 5-(3-((1-(3,6-dimethyl-4-oxo-2-(piperidin-1-yl)-4H-chromen-8-yl)ethyl)amino)pyridin-2-yl)-2-hydroxybenzaldehyde

To a solution of 8-[1-[(2-bromo-3-pyridyl)amino]ethyl]-3,6-dimethyl-2-(1-piperidyl)chromen-4-one (1.60 g, 3.51 mmol, 1 eq), 2-hydroxy-5-(4,4,5,5-tetramethyl-1,3,2-dioxaborolan-2-yl)benzaldehyde (1.74 g, 7.01 mmol, 2 eq) and K₃PO₄ (1.49 g, 7.01 mmol, 2 eq) in dioxane (15 mL)/H₂O (3 mL) was added Pd(PPh₃)₄ (810 mg, 701 μmol, 0.2 eq) in one portion at 25° C., the reaction mixture was heated to 95° C. and stirred at 95° C. for 1 h. The reaction mixture was cooled to 25° C., diluted with H₂O (30 mL) and extracted with EtOAc (20 mL×3). The combined organic layers were washed with brine (20 mL×2), dried over Na₂SO₄, filtered and concentrated under reduced pressure to give a residue. The residue was purified by flash silica gel chromatography (Biotage®; 40 g SepaFlash® Silica Flash Column, Eluent of 0~50% Ethyl acetate/Petroleum ether gradient @60 mL/min). The organic phase was concentrated under reduced pressure to give the title compound (1.60 g, 3.22 mmol, 91.72% yield) as a yellow solid. ¹H NMR (DMSO-d₆, 400 MHz) δ 10.94 (s, 1H), 10.32 (s, 1H), 8.06 (s, 1H), 8.05-8.03 (m, 2H), 7.59-7.57 (m, 1H), 7.35-7.32 (m, 1H), 7.12 (d, J=8.8 Hz, 1H), 7.02-7.00 (m, 1H), 6.86 (d, J=8.8 Hz, 1H), 5.38 (d, J=7.2 Hz, 1H), 4.942 (t, J=6.8 Hz, 1H), 3.25-3.24 (m, 4H), 2.32 (s, 3H), 1.90 (s, 3H), 1.59 (s, 6H), 1.50 (d, J=6.8 Hz, 3H).

### Preparation of 4-(3-((1-(3,6-dimethyl-4-oxo-2-(piperidin-1-yl)-4H-chromen-8-yl)ethyl)amino)pyridin-2-yl)-2-formylphenyl trifluoromethanesulfonate

A mixture of 5-[3-[1-[3,6-dimethyl-4-oxo-2-(1-piperidyl)chromen-8-yl]ethylamino]-2-pyridyl]-2-hydroxy-benzaldehyde (1.50 g, 3.01 mmol, 1 eq), 1,1,1-trifluoro-N-phenyl-N-(trifluoro methylsulfonyl)methanesulfonamide (2.15 g, 6.03 mmol, 2 eq), DMAP (37 mg, 301 μmol, 0.1 eq) and TEA (610 mg, 6.03 mmol, 839 μL, 2 eq) in DCM (30 mL) at 25° C. was degassed and purged with N₂ for 3 times, and then the reaction mixture was stirred at 25° C. for 1 h under N₂ atmosphere. The reaction mixture was concentrated under reduced pressure and the residue was purified by flash silica gel chromatography (Biotage®); 40 g SepaFlash® Silica Flash Column, Eluent of 0~50% Ethyl acetate/Petroleum ether gradient @60 mL/min) to give the title compound (1.20 g, 1.91 mmol, 63.22% yield) as a yellow solid. ¹H NMR (DMSO-d₆, 400 MHz) δ 10.15 (s, 1H), 8.51 (d, J=2.0 Hz, 1H), 8.30 (d, J=10.8 Hz, 1H), 7.96 (d, J=3.2 Hz, 1H), 7.71 (d, J=8.4 Hz, 1H), 7.62 (s, 1H), 7.49 (s, 1H), 7.11-7.09 (m, 1H), 6.85 (d, J=7.6 Hz, 1H), 5.93 (d, J=6.8 Hz, 1H), 4.97 (t, J=6.4 Hz, 1H), 3.35-3.32 (m, 4H), 2.31 (s, 3H), 1.91 (s, 3H), 1.63-1.61 (m, 6H), 1.53 (d, J=5.2 Hz, 3H).

### Preparation of 5-(3-((1-(3,6-dimethyl-4-oxo-2-(piperidin-1-yl)-4H-chromen-8-yl)ethyl) amino)pyridin-2-yl)-2-(4,4,5,5-tetramethyl-1,3,2-dioxaborolan-2-yl)benzaldehyde

A mixture of [4-[3-[1-[3,6-dimethyl-4-oxo-2-(1-piperidyl)chromen-8-yl]ethylamino]-2-pyridyl]-2-formyl-phenyl]trifluoromethanesulfonate (1.10 g, 1.75 mmol, 1 eq), B₂Pin₂ (2.22 g, 8.74 mmol, 5 eq), KOAc (343 mg, 3.49 mmol, 2 eq) and Pd(PPh₃)₂Cl₂ (122 mg, 175 μmol, 0.1 eq) in dioxane (20 mL) was degassed and purged with N₂ for 3 times at 25° C., and then the reaction mixture was stirred at 80° C. for 1 h under N₂ atmosphere. The reaction mixture was quenched by H₂O (20 mL) at 0° C., and then extracted with EtOAc (15 mL×3). The combined organic layers were washed with brine (10 mL×3), dried over Na₂SO₄, filtered and concentrated under reduced pressure to give a residue. The residue was purified by flash silica gel chromatography (Biotage®; 40 g SepaFlash® Silica Flash Column, Eluent of 0~50% Ethyl acetate/Petroleum ether gradient @60 mL/min) to the title compound (960 mg, crude) as a yellow oil, which was used directly for next step without further purification.

### Preparation of 8-(1-((2-(1-hydroxy-JH-benzo[d][1,2,6]oxazaborinin-6-yl)pyridin-3-yl) amino)ethyl)-3,6-dimethyl-2-(piperidin-1-yl)-4H-chromen-4-one

To a solution of 5-[3-[1-[3,6-dimethyl-4-oxo-2-(1-piperidyl)chromen-8-yl]ethylamino]-2-pyridyl]-2-(4,4,5,5-tetramethyl-1,3,2-dioxaborolan-2-yl)benzaldehyde (900 mg, 1.48 mmol, 1 eq) and hydroxylamine;hydrochloride (206 mg, 2.96 mmol, 2 eq) in EtOH (9 ml) was added KOAc (291 mg, 2.96 mmol, 2 eq) in one portion, the reaction mixture was stirred at 25° C. for 1 h. The reaction mixture was diluted with H₂O (5 mL) and the resulting suspension was filtered. The filter cake was washed with MeCN (5 mL) and dried in vacuo to give the title compound (400 mg, 766 μmol, 51.69% yield) as a white solid.

### Preparation of 8-[(1S)-1-[[2-(1-hydroxy-2,3,1-benzoxazaborinin-6-yl)-3-pyridyl]amino]ethyl]-3,6-dimethyl-2-(1-piperidyl)chromen-4-one and 8-[(R)-1-[[2-(1-hydroxy-2,3,1-benzoxazaborinin-6-yl)-3-pyridyl]amino]ethyl]-3,6-dimethyl-2-(1-piperidyl)chromen-4-one

The product was further separated by SFC (column: Chiral Pak IH, 250×30 mm, 10 um; mobile phase: [CO₂-EtOH]; B %:38%, isocratic elution mode) to give the title compound, Isomer 1 (142 mg, 271.82 μmol, 35.50% yield) as a white solid and the title compound, Isomer 2 (120 mg, 230 μmol, 30.00% yield) as a white solid. Isomer 1: ¹H NMR (DMSO-d₆, 400 MHz) δ 9.46 (s, 1H), 8.71 (s, 1H), 8.17 (d, J=8.4 Hz, 1H), 8.09 (d, J=7.2 Hz, 2H), 7.95 (d, J=3.2 Hz, 1H), 7.61 (s, 1H), 7.51 (s, 1H), 7.12-7.10 (m, 1H), 6.93 (d, J=8.0 Hz, 1H), 5.59 (d, J=7.6 Hz, 1H), 4.93 (t, J=6.8 Hz, 1H), 3.25-3.17 (m, 4H), 2.32 (s, 3H), 1.89 (s, 3H), 1.57-1.56 (m, 6H), 1.50 (d, J=6.8 Hz, 3H). MS (ESI): mass calcd. For C₃₀H₃₁BN₄O₄ 522.24, m/z found 523.1 [M+H]⁺. HPLC: 98.43% (220 nm), 98.26% (254 nm). Chiral purity: 100 ee %. Isomer 2: ¹H NMR (DMSO-d₆, 400 MHz) δ 9.46 (s, 1H), 8.71 (s, 1H), 8.17 (d, J=8.4 Hz, 1H), 8.09 (d, J=7.2 Hz, 2H), 7.95 (d, J=3.2 Hz, 1H), 7.61 (s, 1H), 7.51 (s, 1H), 7.12-7.10 (m, 1H), 6.93 (d, J=8.0 Hz, 1H), 5.59 (d, J=7.6 Hz, 1H), 4.93 (t, J=6.8 Hz, 1H), 3.22-3.20 (m, 4H), 2.32 (s, 3H), 1.89 (s, 3H), 1.57-1.56 (m, 6H), 1.51 (d, J=6.8 Hz, 3H). MS (ESI): mass calcd. For C₃₀H₃₁BN₄O₄ 522.24, m/z found 523.2 [M+H]⁺. HPLC: 98.84% (220 rn), 99.02% (254 nm). Chiral purity: 98.92 ee %

### Example 66

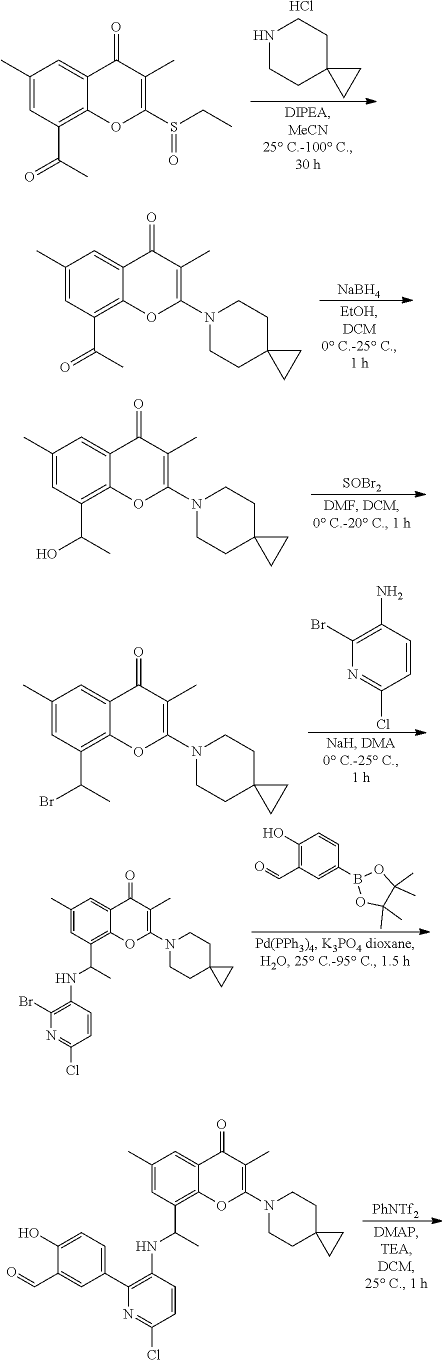

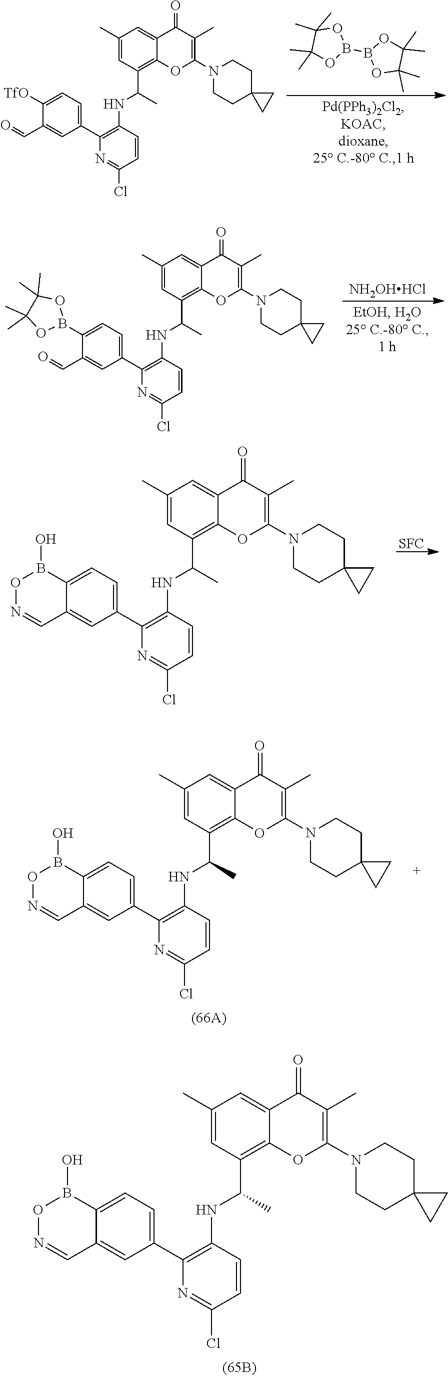

### Preparation of 8-acetyl-2-(6-azaspiro[2.5]octan-6-yl)-3,6-dimethyl-chromen-4-one

To a solution of 8-acetyl-2-ethylsulfinyl-3,6-dimethyl-chromen-4-one (9.00 g, 30.8 mmol, 1 eq) and DIEA (154 mmol, 26.8 mL, 5 eq) in MeCN (100 mL) was added 6-azaspiro octane;hydrochloride (9.09 g, 61.6 mmol, 2 eq) in portions at 25° C., the reaction mixture was directly stirred at 100° C. (the oil bath was pre-heated to 100° C.) for 30 h. The reaction mixture was cooled to 25° C., concentrated under reduced pressure to give a residue. The residue was triturated with MeCN (100 mL)/H₂O (15 mL) to give the title compound (8.50 g, 26.12 mmol, 84.85% yield) as a yellow solid. ¹H NMR (CDCl₃, 400 MHz) δ 8.19 (s, 1H), 7.79 (d, J=2.4 Hz, 1H), 3.53-3.48 (m, 4H), 2.73 (s, 3H), 2.47 (s, 3H), 2.08 (s, 3H), 1.57-1.51 (m, 4H), 0.42 (s, 4H).

### Preparation of 2-(6-azaspiro[2.5]octan-6-yl)-8-(1-hydroxyethyl)-3,6-dimethyl-chromen-4-one

To a solution of 8-acetyl-2-(6-azaspiro[2.5]octan-6-yl)-3,6-dimethyl-chromen-4-one (7.00 g, 21.5 mmol, 1 eq) in EtOH (30 mL)/DCM (40 mL) was added NaBH₄ (1.82 g, 48.1 mmol, 2.24 eq) in portions at 0° C. under N₂, the reaction mixture was warmed to 25° C. and stirred for 1 h. The reaction mixture was poured into ice water (100 mL) at 0° C. and nitrogen atmosphere protection was applied. Then the reaction mixture was warmed up to 25° C. and extracted with DCM (60 mL×3). The combined organic phase was washed with brine (70 mL×2), dried over Na₂SO₄, filtered and concentrated under reduced pressure to give the title compound (7.00 g, 21.38 mmol, 99.38% yield) as a yellow solid, which was used directly for the next step without purification. ¹H NMR (DMSO-d₆, 400 MHz) δ 7.63 (s, 1H), 7.58 (d, J=2.0 Hz, 1H), 5.33 (d, J=4.0 Hz, 1H), 5.26-5.16 (m, 1H), 3.44-3.38 (m, 4H), 2.39 (s, 3H), 1.91 (s, 3H), 1.51-1.46 (m, 4H), 1.40 (d, J=6.4 Hz, 3H), 0.38 (s, 4H).

### Preparation of 2-(6-azaspiro[2.5]octan-6-yl)-8-(1-bromoethyl)-3,6-dimethyl-chromen-4-one

To a solution of 2-(6-azaspiro[2.5]octan-6-yl)-8-(1-hydroxyethyl)-3,6-dimethyl-chromen-4-one (1.50 g, 4.58 mmol, 1 eq) and DMF (218 mg, 2.98 mmol, 229 μL, 0.65 eq) in DCM (25 mL) was added thionylBromide (1.24 g, 5.96 mmol, 461 μL, 1.3 eq) dropwise at 0° C., the reaction mixture was warmed to 20° C. and stirred for 1 h. The reaction mixture was poured into ice- H₂O (20 mL) at 0° C. and extracted with DCM (20 mL×3). The combined organic layers were washed with sat.aq NaHCO₃ to pH=8, washed with brine (30 mL), dried over Na₂SO₄, filtered and concentrated under reduced pressure to give the title compound (1.60 g, 4.10 mmol, 89.48% yield) as a yellow solid was used directly for the next step without purification. ¹H NMR (CDCl₃, 400 MHz) δ 7.89 (s, 1H), 7.45 (s, 1H), 5.59 (q, J=6.8 Hz, 1H), 3.50-3.45 (m, 4H), 2.37 (s, 3H), 2.07 (d, J=7.2 Hz, 3H), 2.00 (s, 3H), 1.53-1.46 (m, 4H), 0.36 (s, 4H).

### Preparation of 2-(6-azaspiro[2.5]octan-6-yl)-8-[1-[(2-bromo-6-chloro-3-pyridyl)amino]ethyl]-3,6-dimethyl-chromen-4-one

The title compound was prepared as a brown solid (1.30 g, 2.52 mmol, 65.45% yield) according to the procedure described in preceding examples, using 2-bromo-6-chloro-pyridin-3-amine (957 mg, 4.61 mmol, 1.2 eq) and 2-(6-azaspiro[2.5]octan-6-yl)-8-(1-bromo ethyl)-3,6-dimethyl-chromen-4-one (1.50 g, 3.84 mmol, 1 eq). ¹H NMR (CDCl₃, 400 MHz) δ 7.92 (s, 1H), 7.29 (d, J=2.0 Hz, 1H), 6.97 (d, J=8.4 Hz, 1H), 6.47 (d, J=8.4 Hz, 1H), 4.95-4.87 (m, 1H), 4.86-4.78 (m, 1H), 3.50-3.44 (m, 4H), 2.38 (s, 3H), 2.09 (s, 3H), 1.70 (d, J=6.8 Hz, 3H), 1.57-1.54 (m, 4H), 0.43 (s, 4H).

### Preparation of 5-[3-[1-[2-(6-azaspiro[2.5]octan-6-yl)-3,6-dimethyl-4-oxo-chromen-8-yl]ethylamino]-6-chloro-2-pyridyl]-2-hydroxy-benzaldehyde

The title compound was prepared as a yellow solid (650 mg, 1.16 mmol, 92.62% yield) according to the procedure described in preceding examples, using 2-(6-azaspiro[2.5]octan-6-yl)-8-[1-[(2-bromo-6-chloro-3-pyridyl)amino]ethyl]-3,6-dimethyl-chromen-4-one (650 mg, 1.26 mmol, 1 eq) and 2-hydroxy-5-(4,4,5,5-tetramethyl-1,3,2-diox aborolan-2-yl)benzaldehyde (343 mg, 1.38 mmol, 1.1 eq). ¹H NMR (CDCl₃, 400 MHz) δ 11.18 (s, 1H), 9.99 (s, 1H), 7.97 (d, J=2.0 Hz, 1H), 7.95-7.91 (m, 2H), 7.28 (d, J=2.0 Hz, 1H), 7.16 (d, J=8.8 Hz, 1H), 7.01 (d, J=8.4 Hz, 1H), 6.65 (d, J=8.8 Hz, 1H), 4.89 (q, J=6.8 Hz, 1H), 3.46-3.34 (m, 4H), 2.39 (s, 3H), 2.10 (s, 3H), 1.25 (s, 6H), 0.41 (s, 4H).

### Preparation of [4-[3-[1-[2-(6-azaspiro[2.5]octan-6-yl)-3,6-dimethyl-4-oxo-chromen-8-yl]ethylamino]-6-chloro-2-pyridyl]-2-formyl-phenyl]trifluoromethanesulfonate

The title compound was prepared as a yellow solid (1.00 g, 1.45 mmol, 80.86% yield) according to the procedure described in preceding examples, using 5-[3-[1-[2-(6-azaspiro[2.5]octan-6-yl)-3,6-dimethyl-4-oxo-chromen-8-yl]ethylamino]-6-chloro-2-pyridyl]-2-hydroxy-benzaldehyde (1.00 g, 1.79 mmol). ¹H NMR (CDCl₃, 400 MHz) δ 10.35 (s, 1H), 8.44 (d, J=2.4 Hz, 1H), 8.13 (dd, J=8.8, 2.4 Hz, 1H), 7.94 (s, 1H), 7.59 (d, J=8.4 Hz, 1H), 7.30 (s, 1H), 7.05 (d, J=8.4 Hz, 1H), 6.68 (d, J=8.8 Hz, 1H), 5.00-4.86 (m, 1H), 4.50-4.25 (m, 1H), 3.50-3.38 (m, 4H), 2.39 (s, 3H), 2.11 (s, 3H), 1.60 (d, J=6.8 Hz, 3H), 1.56-1.52 (m, 4H), 0.43 (s, 4H).

### Preparation of 5-[3-[1-[2-(6-azaspiro[2.5]octan-6-yl)-3,6-dimethyl-4-oxo-chromen-8-yl]ethylamino]-6-chloro-2-pyridyl]-2-(4,4,5,5-tetramethyl-1,3,2-dioxaborolan-2-yl)benzaldehyde

The title compound was prepared as yellow oil (350 mg, 523.93 μmol, 72.32% yield) according to the procedure described in preceding examples, using [4-[3-[1-[2-(6-azaspiro[2.5]octan-6-yl)-3,6-dimethyl-4-oxo-chromen-8-yl]ethylamino]-6-chloro-2-pyridyl]-2-formyl-phenyl]trifluoromethanesulfonate (500 mg, 725 μmol, 1 eq) and B₂Pin₂ (736 mg, 2.90 mmol, 4 eq). ¹H NMR (CDCl₃, 400 MHz) δ 10.62 (s, 1H), 8.34 (s, 1H), 8.04 (d, J=7.6 Hz, 1H), 7.95 (dd, J=7.6, 1.6 Hz, 1H), 7.92 (s, 1H), 7.28 (d, J=2.0 Hz, 1H), 7.02 (d, J=8.8 Hz, 1H), 6.64 (d, J=8.8 Hz, 1H), 4.98-4.83 (m, 1H), 4.55-4.40 (m, 1H), 3.47-3.36 (m, 4H), 2.39 (s, 3H), 2.10 (s, 3H), 1.53 (d, J=6.4 Hz, 4H), 1.51 (d, J=5.6 Hz, 3H), 1.25 (s, 12H), 0.41 (s, 4H).

### Preparation of 2-(6-azaspiro[2.5]octan-6-yl)-8-[1-[[6-chloro-2-(1-hydroxy-2,3,1-benzoxazaborinin-6-yl)-3-pyridyl]amino]ethyl]-3,6-dimethyl-chromen-4-one

The title compound was prepared as an off white solid (250 mg, 428.90 μmol, 47.75% yield) according to the procedure described in preceding examples, using 5-[3-[1-[2-(6-azaspiro[2.5]octan-6-yl)-3,6-dimethyl-4-oxo-chromen-8-yl]ethylamino]-6-chloro-2-pyridyl]-2-(4,4,5,5-tetramethyl-1,3,2-dioxaborolan-2-yl)benzaldehyde (600 mg, 898 mol). ¹H NMR (DMSO-d₆, 400 MHz) δ 9.48 (s, 1H), 8.72 (s, 1H), 8.16 (d, J=8.0 Hz, 1H), 8.10-8.01 (m, 2H), 7.62 (s, 1H), 7.46 (d, J=2.0 Hz, 1H), 7.02 (d, J=8.8 Hz, 1H), 6.99 (d, J=9.2 Hz, 1H), 5.83 (d, J=7.2 Hz, 1H), 4.95-4.85 (m, 1H), 3.46-3.36 (m, 4H), 2.32 (s, 3H), 1.91 (s, 3H), 1.50 (d, J=6.4 Hz, 3H), 1.45-1.39 (m, 4H), 0.35 (s, 4H).

### Preparation of 2-(6-azaspiro[2.5]octan-6-yl)-8-[(1S)-1-[[6-chloro-2-(1-hydroxy-2,3,1-benzoxazaborinin-6-yl)-3-pyridyl]amino]ethyl]-3,6-dimethyl-chromen-4-one and 2-(6-azaspiro[2.5]octan-6-yl)-8-[(1R)-1-[[6-chloro-2-(1-hydroxy-2,3,1-benzoxazaborinin-6-yl)-3-pyridyl]amino]ethyl]-3,6-dimethyl-chromen-4-one

The crude product (400 mg) was further separated by SFC (column: DAICEL CHIRALCEL OD (250 mm×50 mm, 10 um); mobile phase: [CO₂—IPA]; B %: 55%, isocratic elution mode) to give the title compound, Isomer 1 (137 mg, 231.21 μmol, 35.46% yield) as a pale-yellow solid and the title compound, Isomer 2 (150 mg, 239.46 μmol, 36.73% yield) as a pale yellow solid. Isomer 2 was further purified by prep-HPLC (column: Phenomenex Gemini-DX 80×40 mm×3 um; mobile phase: [H₂O (10 mM NH₄HCO₃)-AC□]; gradient: 15%-45% B over 8.0 min) to give a white solid (100.2 mg, 171.32 μmol, 26.28% yield). Isomer 1: ¹H NMR (DMSO-d₆, 400 MHz) δ 9.48 (s, 1H), 8.72 (s, 1H), 8.16 (d, J=8.0 Hz, 1H), 8.08-8.04 (m, 2H), 7.63 (s, 1H), 7.46 (d, J=2.0 Hz, 1H), 7.19 (d, J=8.8 Hz, 1H), 6.99 (d, J=8.4 Hz, 1H), 5.83 (d, J=7.2 Hz, 1H), 5.05-4.75 (m, 1H), 3.30-3.22 (m, 4H), 2.32 (s, 3H), 1.91 (s, 3H), 1.50 (d, J=6.8 Hz, 3H), 1.45-1.40 (m, 4H), 0.35 (s, 4H). MS (ESI): mass calcd. For C₃₂H₃₂BClN₄O₄ 582.22, m/z found 583.1 [M+H]⁺. HPLC: 98.37 (220 nm), 99.20 (254 nm). Chiral purity: 100% ee. Isomer 2: ¹H NMR (DMSO-d₆, 400 MHz) δ 9.50-9.22 (m, 1H), 8.71 (s, 1H), 8.16 (d, J=7.2 Hz, 1H), 8.09-8.00 (m, 2H), 7.62 (s, 1H), 7.46 (d, J=2.0 Hz, 1H), 7.19 (d, J=8.4 Hz, 1H), 6.98 (d, J=8.8 Hz, 1H), 5.83 (d, J=7.2 Hz, 1H), 4.96-4.86 (m, 1H), 3.31-3.26 (m, 4H), 2.32 (s, 3H), 1.91 (s, 3H), 1.50 (d, J=6.8 Hz, 3H), 1.45-1.39 (m, 4H), 0.35 (s, 4H). MS (ESI): mass calcd. For C₃₂H₃₂BClN₄O₄ 582.22, m/z found 583.1 [M+H]⁺. HPLC: 99.66 (220 nm), 100.00 (254 nm). Chiral purity: 97.86% ee

### Example 67

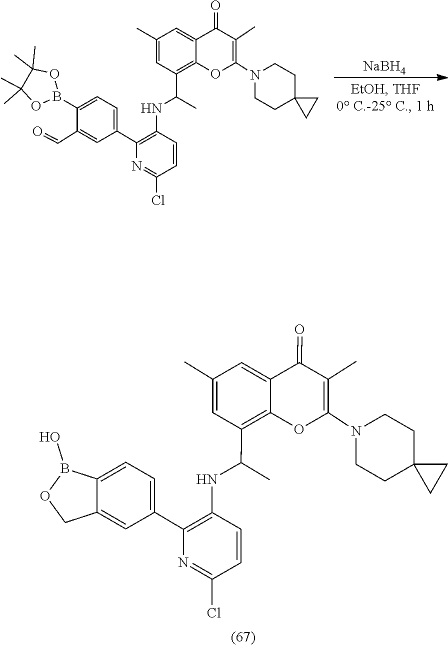 [SMILES: Cc1cc(C(C)Nc2ccc(Cl)nc2-c2ccc(B3OC(C)(C)C(C)(C)O3)c(C=O)c2)c2oc(N3CCC4(CC3)CC4)c(C)c(=O)c2c1 ; "NaBH4" ; Cc1cc(C(C)Nc2ccc(Cl)nc2-c2ccc3c(c2)COB3O)c2oc(N3CCC4(CC3)CC4)c(C)c(=O)c2c1]

### Preparation of 8-(1-((6-chloro-2-(1-hydroxy-1,3-dihydrobenzo[c][1,2]oxaborol-5-yl)pyridin-3-yl)amino)ethyl)-3,6-dimethyl-2-(6-azaspiro[2.5]octan-6-yl)-4H-chromen-4-one

To a solution of 5-[3-[1-[2-(6-azaspiro[2.5]octan-6-yl)-3,6-dimethyl-4-oxo-chromen-8-yl]ethylamino]-6-chloro-2-pyridyl]-2-(4,4,5,5-tetramethyl-1,3,2-dioxaborolan-2-yl)benzaldehyde (100 mg, 150 μmol, 1 eq) in THF (4 mL)/EtOH (0.1 mL) was added NaBH₄ (50 mg, 1.32 mmol, 8.83 eq) in portions at 0° C., the reaction mixture was stirred at 25° C. for 1 h. The reaction mixture was poured into H₂O (6 mL) at 0° C., adjusted pH=6 with 2N HCl and extracted with EtOAc (8 mL×3). The combined organic layers were washed with brine (5 mL), dried over Na₂SO₄, filtered and concentrated under reduced pressure to give a residue. The residue was purified by prep-HPLC (column: Phenomenex Gemini-NX 80×40 mm×3 um; mobile phase: [H₂O (10 mM NH₄HCO₃)-ACN]; gradient:35%-65% B over 8.0 min) to give the title compound (18.9 mg, 32.84 μmol, 21.94% yield) as a white solid. ¹H NMR (DMSO-d₆, 400 MHz) δ 9.28 (s, 1H), 7.84 (d, J=8.0 Hz, 1H), 7.68-7.64 (m, 2H), 7.63 (s, 1H), 7.45 (d, J=2.0 Hz, 1H), 7.15 (d, J=8.8 Hz, 1H), 6.95 (d, J=8.8 Hz, 1H), 5.51 (d, J=7.2 Hz, 1H), 5.07-4.97 (m, 2H), 4.96-4.88 (m, 1H), 3.30-3.26 (m, 4H), 2.32 (s, 3H), 1.91 (s, 3H), 1.50 (d, J=6.4 Hz, 3H), 1.45~1.40 (m, 4H), 0.35 (s, 4H). MS (ESI): mass calcd. For C₃₂H₃₃BClN₃O₄ 569.23, m/z found 570.1 [M+H]⁺. HPLC: 99.03 (220 nm), 100.00 (254 nm).

### Example 68

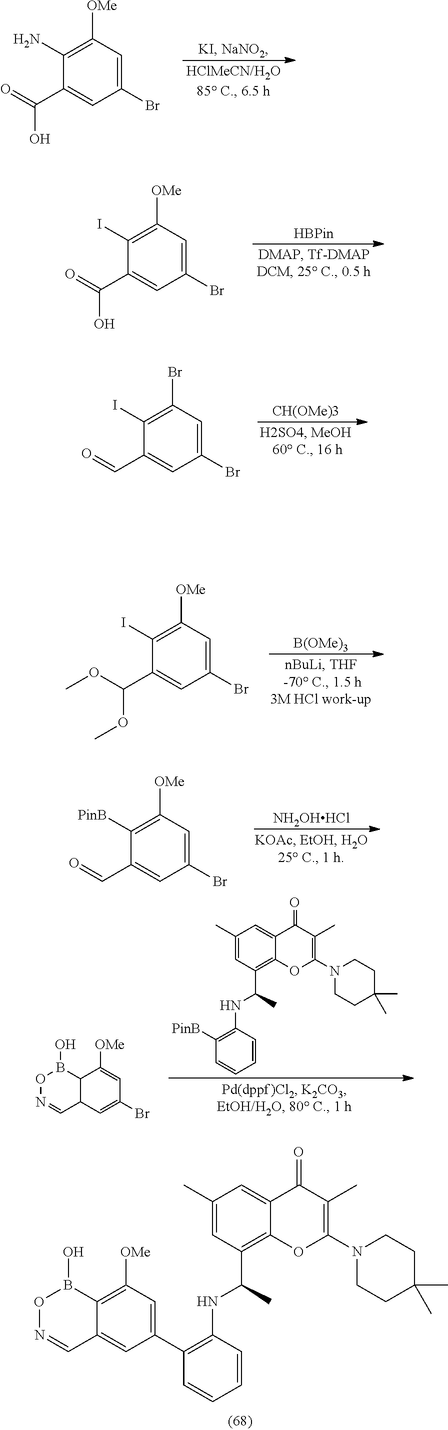

### Preparation of methyl 5-bromo-2-iodo-3-methoxy-benzoic acid

To a suspension of 2-amino-5-bromo-3-methoxy-benzoic acid (8.50 g, 34.54 mmol, 1 eq) in H₂O (40 mL)/MeCN (40 mL)/HCl (12 M, 17.27 mL, 6 eq) was added dropwise a solution of NaNO₂ (4.77 g, 69.09 mmol, 2 eq) in H₂O (40 mL) at 0° C., the reaction mixture was stirred at 0° C. for 2 h. Then KI (11.47 g, 69.09 mmol, 2 eq) was added in portions and the resulting reaction mixture was stirred at 0~85° C. for 4.5 h. The reaction mixture was cooled to 25° C. and concentrated under reduced pressure to remove MeCN. The solid was collected by filtration, washed with H₂O (10 mL×5) and dried in vacuo to give the title compound (10.20 g, 28.58 mmol, 82.72% yield) as a white solid. ¹H NMR (DMSO-d₆, 400 MHz) δ 13.56 (br s, 1H), 7.30 (d, J=2.0 Hz, 1H), 2.27 (d, J=2.0 Hz, 1H), 3.89 (s, 3H).

### Preparation of 5-bromo-2-iodo-3-methoxy-benzaldehyde

To a mixture of 5-bromo-2-iodo-3-methoxy-benzoic acid (10.20 g, 28.58 mmol, 1 eq), N,N-dimethyl-1-(trifluoromethylsulfonyl)pyridin-1-ium-4-amine;trifluoromcthanesulfonate (23.11 g, 57.15 mmol, 2 eq) and DMAP (6.98 g, 57.15 mmol, 2 eq) in DCM (100 mL) was added HBPin (3.66 g, 28.58 mmol, 4.15 mL, 1 eq) dropwise at 25° C., the reaction mixture was stirred at 25° C. for 0.5 h. The reaction mixture was filtered to remove the insoluble impurity and the filtrate was quenched by H₂O (50 mL) and extracted with DCM (50 mL×3). The combined organic layers were washed with H₂O (50 mL×2), dried over Na₂SO₄, filtered and concentrated under reduced pressure. The residue was purified by flash silica gel chromatography (Biotage®; 80 g SepaFlash® Silica Flash Column, Eluent of 0~20% Ethyl acetate/Petroleum ether gradient @100 mL/min). The eluent was concentrated under reduced pressure to give crude product. The crude product was further purified by flash silica gel chromatography (Agela C18; 330 g SepaFlash® MeCN/H₂O gradient 55-75% 25 min; 75% 10 min @70 mL/min) to give the title compound (3.14 g, 9.21 mmol, 32.23% yield) as an off-white solid. ¹H NMR (DMSO-d₆, 400 MHz) δ 10.00 (s, 1H), 7.48 (d, J=2.0 Hz, 1H), 7.43 (d, J=2.0 Hz, 1H), 3.94 (s, 3H).

### Preparation of 5-bromo-1-(dimethoxymethyl)-2-iodo-3-methoxy-benzene

To a mixture of 5-bromo-2-iodo-3-methoxy-benzaldehyde (3.14 g, 9.21 mmol, 1 eq) and trimethoxymethane (1.95 g, 18.42 mmol, 2.02 mL, 2 eq) in MeOH (30 mL) was added H₂SO₄ (90 mg, 920.98 gmol, 49.09 μL, 0.1 eq) dropwise at 25° C., then the reaction mixture was heated to 60° C. and stirred at 60° C. for 16 h. The reaction mixture was cooled to 20° C., poured into sat.aq.Na₂CO₃ (30 mL) at 0° C. and extracted with EtOAc (50 mL×3). The combined organic layers were washed with brine (50 mL), dried over Na₂SO₄, filtered and concentrated under reduced pressure to give the title compound (3.25 g, 8.40 mmol, 91.18% yield) as yellow oil. ¹H NMR (DMSO-d₆, 400 MHz) δ 7.20 (d, J=2.0 Hz, 1H), 7.16 (d, J=2.0 Hz, 1H), 3.35 (s, 1H), 3.87 (s, 3H), 3.29 (s, 6H).

### Preparation of 5-bromo-3-methoxy-2-(4,4,5,5-tetramethyl-1,3,2-dioxaborolan-2-yl)benzaldehyde

To a solution of 5-bromo-1-(dimethoxymethyl)-2-iodo-3-methoxy-benzene (1.50 g, 3.88 mmol, 1 eq) in THF (15 mL) was added n-BuLi (2.5 M, 1.55 mL, 1 eq) dropwise at −70° C. under N₂, the reaction mixture was stirred at −70° C. for 0.5 h. Then trimethyl borate (402.75 mg, 3.88 mmol, 437.77 μL, 1 eq) was added dropwise at −70° C. and the resulting reaction mixture was stirred at −70° C. for 1 h. The reaction mixture was quenched by addition aq. HCl (3M, 30 mL) and extracted with EtOAc (30 mL×3).

The combined organic layers were washed with brine (30 mL), dried over Na₂SO₄, filtered and concentrated under reduced pressure. The crude product was further triturated with Petroleum ether (15 mL) at 25° C. for 30 min to give the title compound (600 mg, crude) as an off-white solid. ¹H NMR (DMSO-d₆, 400 MHz) δ 9.87 (s, 1H), 8.02 (s, 2H), 7.65 (s, 1H), 7.42 (s, 1H), 3.79 (s, 3H).

### Preparation of 6-bromo-1-hydroxy-8-methoxy-2,3,1-benzoxazaborinine

To a mixture of 5-bromo-3-methoxy-2-(4,4,5,5-tetramethyl-1,3,2-dioxaborolan-2-yl)benzaldehyde (450 mg, 1.74 mmol, 1 eq) and KOAc (342 mg, 3.48 mmol, 2 eq) in EtOH (5 mL)/H₂O (1 mL) was added NH₂OH·HCl (182 mg, 2.61 mmol, 1.5 eq) at 25° C., the reaction mixture was stirred at 25° C. for 1 h. The reaction mixture was directly filtered, the filtered cake was washed with H₂O (5 mL×2) and dried in vacuo to give the title compound (450 mg, crude) as a white solid, which was used directly for next step without further purification.

### Preparation of 2-(4,4-dimethyl-1-piperidyl)-8-[(1R)-1-[2-(1-hydroxy-8-methoxy-2,3,1-benzoxazaborinin-6-yl)anilino]ethyl]-3,6-dimethyl-chromen-4-one

To a mixture of 2-(4,4-dimethyl-1-piperidyl)-3,6-dimethyl-8-[(1R)-1-[2-(4,4,5,5-tetramethyl-1,3,2-dioxaborolan-2-yl)anilino]ethyl]chromen-4-one (200 mg, 377.00 μmol, 1 eq), 6-bromo-1-hydroxy-8-methoxy-2,3,1-benzoxazaborinine (126 mg, 490.10 μmol, 1.3 eq) and K₂CO₃ (105 mg, 754.00 gmol, 2 eq) in EtOH (3 mL)/H₂O (0.6 mL) was added Pd(dppf)Cl₂ (28 mg, 37.70 μmol, 0.1 eq).

The reaction mixture was heated to 80° C. and stirred at 80° C. for 1 h under N₂. The reaction mixture was cooled to 20° C., filtered and the filtrate was directly purified by prep-HPLC (column: Waters Xbridge BEH C18 100×30 mm×10 um; mobile phase: [H₂O (10 mM NH₄HCO₃)-MeOH]; gradient:70%-95% B over 8.0 min) to give the title compound (70.8 mg, 121.78 μmol, 32.30% yield) as an off-white solid. ¹H NMR (DMSO-d₆, 400 MHz) δ 8.58 (s, 1H), 8.36 (br s, 1H), 7.61 (s, 1H), 7.48 (s, 1H), 7.36 (d, J=10.8 Hz, 2H), 7.11-7.09 (m, 2H), 6.70 (t, J=7.2 Hz, 1H), 6.55 (d, J=8.4 Hz, 1H), 5.16 (d, J=7.2 Hz, 1H), 5.01-4.94 (m, 1H), 3.86 (s, 3H), 3.29-3.24 (m, 4H), 2.32 (s, 3H), 1.90 (s, 3H), 1.49 (d, J=5.6 Hz, 3H), 1.40 (t, J=5.2 Hz, 4H), 0.95 (s, 6H). MS (ESI): mass calcd. For C₃₄H₃₈BN₃O₅ 579.29, m/z found 580.2 [M+H]⁺. HPLC: 99.67% (220 nm), 99.62% (254 nm). Chiral purity: 99.74% ee.

### Example 69

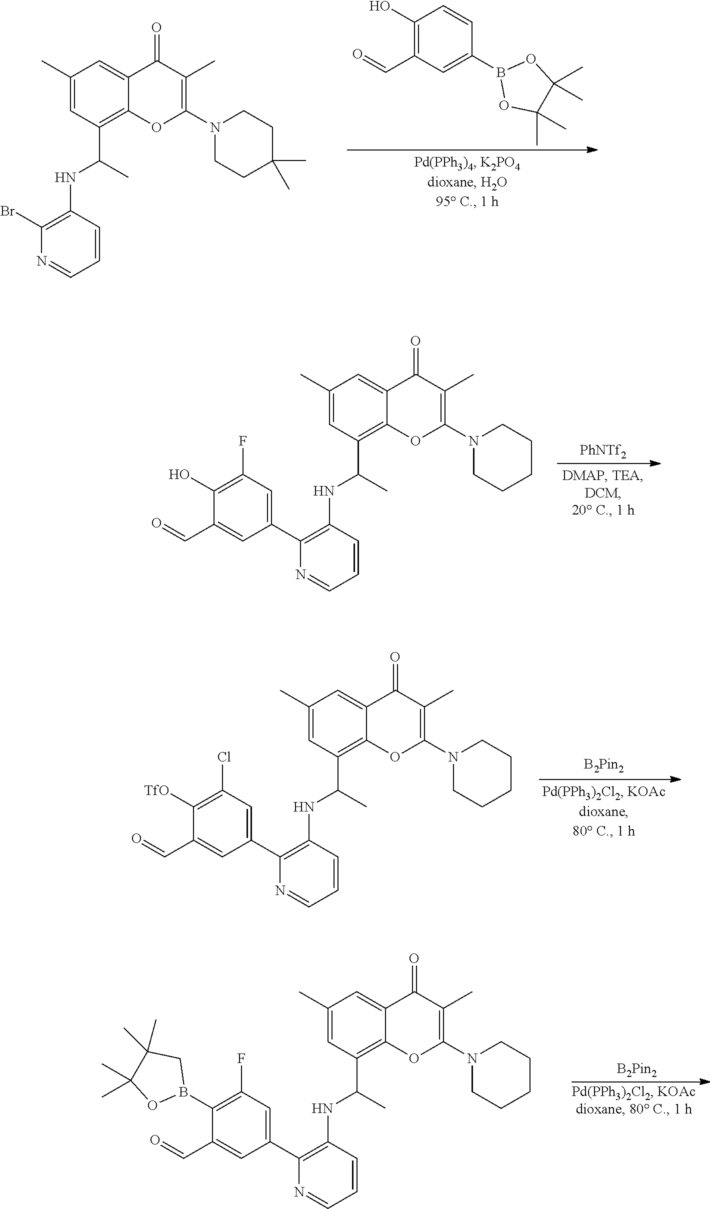

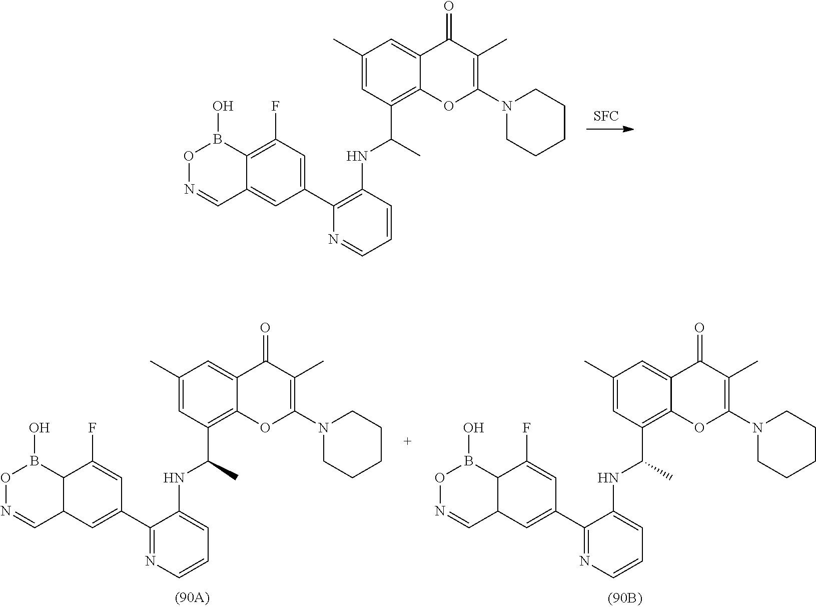

### Preparation of 5-(3-((1-(3,6-dimethyl-4-oxo-2-(piperidin-1-yl)-4H-chromen-8-yl)ethyl)amino)pyridin-2-yl)-3-fluoro-2-hydroxybenzaldehyde

The title compound was prepared as a yellow solid (0.3 g, 581.88 μmol, 26.56% yield) according to the procedures described herein and the synthetic scheme, using 8-(1-((2-bromopyridin-3-yl)amino)ethyl)-3,6-dimethyl-2-(piperidin-1-yl)-4H-chromen-4-one (1.00 g, 2.19 mmol, 1 eq) and 3-fluoro-2-hydroxy-5-(4,4,5,5-tetramethyl-1,3,2-dioxaborolan-2-yl)benzaldehyde (874 mg, 3.29 mmol, 1.5 eq). ¹H NMR (CDCl₃, 400 MHz) δ 11.14-11.08 (m, 1H), 10.01 (d, J=1.6 Hz, 1H), 8.05 (d, J=4.4 Hz, 1H), 7.89 (d, J=1.6 Hz, 1H), 7.83-7.80 (m, 2H), 7.31-7.30 (m, 1H), 7.05-7.03 (m, 1H), 6.74-6.72 (m, 1H), 4.95-4.90 (m, 1H), 4.43-4.42 (m, 1H), 3.36-3.28 (m, 4H), 2.38 (s, 3H), 2.05 (s, 3H), 1.69 (s, 6H), 1.60 (d, J=6.8 Hz, 3H).

### Preparation of 4-(3-((1-(3,6-dimethyl-4-oxo-2-(piperidin-1-yl)-4H-chromen-8-yl)ethyl)amino)pyridin-2-yl)-2-fluoro-6-formylphenyl trifluoromethanesulfonate

The title compound was prepared as yellow solid (550 mg, 849.24 μmol, 62.55% yield) according to the procedures described herein and the synthetic scheme, using 5-(3-((1-(3,6-dimethyl-4-oxo-2-(piperidin-1-yl)-4H-chromen-8-yl)ethyl)amino)pyridin-2-yl)-3-fluoro-2-hydroxybenzaldehyde (700 mg, 1.36 mmol, 1 eq) and PhNTf₂ (970 mg, 2.72 mmol, 2 eq). ¹H NMR (CDCl₃, 400 MHz) δ 10.29 (s, 1H), 8.27 (d, J=1.6 Hz, 1H), 8.08-8.07 (m, 1H), 8.06-8.01 (m, 1H), 7.90-7.89 (m, 1H), 7.33 (s, 1H), 7.07-7.05 (m, 1H), 6.75-6.73 (m, 1H), 5.01-4.95 (m, 1H), 4.44-4.43 (m, 1H), 3.36-3.35 (m, 4H), 2.38 (s, 3H), 2.06 (s, 3H), 1.70 (s, 6H), 1.61 (d, J=6.8 Hz, 3H).

### Preparation of 5-(3-((1-(3,6-dimethyl-4-oxo-2-(piperidin-1-yl)-4H-chromen-8-yl)ethyl)amino)pyridin-2-yl)-3-fluoro-2-(4,4,5,5-tetramethyl-1,3,2-dioxaborolan-2-yl)benzaldehyde

The title compound was prepared as a light yellow gum (330 mg, 637.21 μmol, 69.76% yield) according to the procedures described herein and the synthetic scheme, using 4-(3-((1-(3,6-dimethyl-4-oxo-2-(piperidin-1-yl)-4H-chromen-8-yl)ethyl)amino)pyridin-2-yl)-2-fluoro-6-formylphenyl trifluoromethanesulfonate (500 mg, 772 μmol, 1 eq) and B₂Pin₂ (980 mg, 3.86 mmol, 5 eq). The product was used directly for next step without further purification.

### Preparation of 8-(1-((2-(8-fluoro-1-hydroxy-1H-benzo[d][1,2,6]oxazaborinin-6-yl) pyridine-3-yl)amino)ethyl)-3,6-dimethyl-2-(piperidin-1-yl)-4H-chromen-4-one

The title compound was prepared as a white solid (180 mg, 333.09 μmol, 41.67% yield) according to the procedures described herein and the synthetic scheme, using 5-(3-((1-(3,6-dimethyl-4-oxo-2-(piperidin-1-yl)-4H-chromen-8-yl)ethyl)amino)pyridine-2-yl)-3-fluoro-2-(4,4,5,5-tetramethyl-1,3,2-dioxaborolan-2-yl)benzaldehyde (500 mg, 799 μmol, 1 eq) and NH₂OH·HCl (111 mg, 1.60 mmol, 2 eq).

### Preparation of (S)-8-(1-((2-(8-fluoro-1-hydroxy-1H-benzo[d][1,2,6]oxazaborinin-6-yl) pyridin-3-yl)amino)ethyl)-3,6-dimethyl-2-(piperidin-1-yl)-4H-chromen-4-one and (R)-8-(1-((2-(8-fluoro-1-hydroxy-1H-benzo[d][1,2,6]oxazaborinin-6-yl)pyridin-3-yl)amino)ethyl)-3,6-dimethyl-2-(piperidin-1-yl)-4H-chromen-4-one

8-(1-((2-(8-fluoro-1-hydroxy-1H-benzo[d][1,2,6]oxazaborinin-6-yl)pyridine-3-yl)amino)ethyl)-3,6-dimethyl-2-(piperidin-1-yl)-4H-chromen-4-one (180 mg, 333.09 μmol, 1 eq) was separated by SFC (column: ChiralPak IH, 250×30 mm, 10 um; mobile phase: [CO2-MeOH]; B %: 50%, isocratic elution mode) to give the title compound, Isomer 1 (68.5 mg, 126.19 μmol, 37.88% yield) as white solid and the title compound, Isomer 2 (68.2 mg, 125.56 μmol, 37.70% yield) as a white solid. Isomer 1: ¹H NMR (DMSO-d₆, 400 MHz) δ 8.26-8.20 (m, 1H), 7.92-7.89 (m, 1H), 7.62-7.47 (m, 3H), 7.47-7.47 (m, 1H), 7.08-7.03 (m, 1H), 6.84-6.74 (m, 1H), 5.47-5.42 (m, 1H), 4.98-4.92 (m, 1H), 3.14-3.08 (m, 4H), 2.31 (s, 3H), 1.90 (s, 3H), 1.59 (s, 6H), 1.51 (d, J=6.4 Hz, 3H). MS (ESI): mass calcd. For C₃₀H₃₀BFN₄O₄ 540.40, m/z found 541.2 [M+H]⁺. HPLC: 99.55% (220 nm), 99.65% (254 nm). Chiral purity: 97.84% ee. Isomer 2: ¹H NMR (DMSO-d₆, 400 MHz) δ 8.20-8.00 (m, 1H), 7.90-7.89 (m, 1H), 7.62-7.46 (m, 1H), 7.40-7.35 (m, 3H), 7.06-7.03 (m, 1H), 6.81-6.79 (m, 1H), 5.45-5.18 (m, 1H), 4.98-4.93 (m, 1H), 3.15-3.12 (m, 4H), 2.31 (s, 3H), 1.90 (s, 3H), 1.60 (s, 6H), 1.51 (s, J=6.4 Hz, 3H). MS (ESI): mass calcd. For C₃₀H₃₀BFN₄O₄ 540.40, m/z found 541.2 [M+H]⁺. HPLC: 99.49% (220 nm), 99.56% (254 nm). Chiral purity: 100% ee.

### Example 70

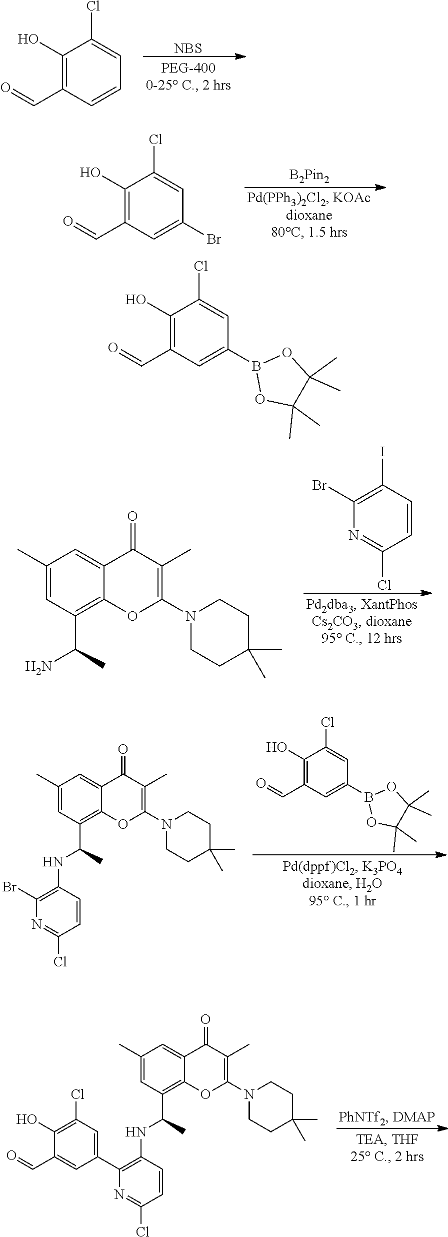

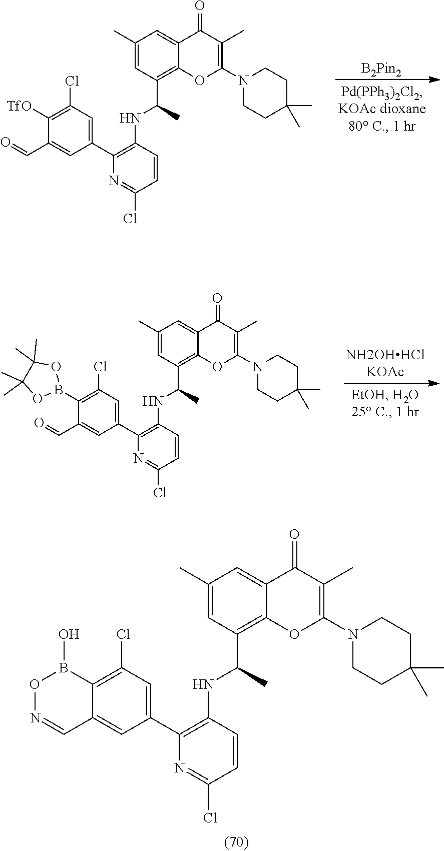

### Preparation of 5-bromo-3-chloro-2-hydroxy-benzaldehyde

To a solution of 3-chloro-2-hydroxy-benzaldehyde (23.00 g, 146.90 mmol, 1 eq) in PEG-400 (500 mL) was added NBS (28.76 g, 161.59 mmol, 1.1 eq) in portions at 0° C., the reaction mixture was warmed to 25° C. and stirred at 25° C. for 2 h. The reaction mixture was cooled to 0° C. and quenched by adding water (500 mL) at 0° C. The resulting reaction suspension was filtered and the filter cake was dried in vacuo to give the title compound (33.00 g, 140.15 mmol, 95.40% yield) as a light-yellow solid. ¹H NMR (CDCl₃, 400 MHz) δ 11.39 (s, 1H), 9.86 (s, 1H), 7.75 (s, 3H), 7.63 (s, 3H).

### Preparation of 3-chloro-2-hydroxy-5-(4,4,5,5-tetramethyl-1,3,2-dioxaborolan-2-yl) benzaldehyde

A mixture of 5-bromo-3-chloro-2-hydroxy-benzaldehyde (3.00 g, 12.74 mmol, 1 eq), B₂Pin₂ (4.21 g, 16.56 mmol, 1.3 eq), KOAc (2.50 g, 25.48 mmol, 2 eq) and Pd(PPh₃)₂Cl₂ (895 mg, 1.27 mmol, 0.1 eq) in dioxane (30 mL) was degassed and purged with N₂ for 3 times, and then the reaction mixture was heated to 80° C. and stirred at 80° C. for 1.5 h under N₂ atmosphere. The reaction mixture was cooled to 25° C., filtered and the filtrate was concentrated under reduced pressure to give a residue. The residue was purified by flash silica gel chromatography (Biotage®; 40 g SepaFlash® Silica Flash Column, Eluent of 0~8% Ethyl acetate/Petroleum ether gradient @60 m/min) to give the title compound (4.90 g, 8.67 mmol, 68.06% yield) as a white solid. ¹H NMR (CDCl₃, 400 MHz) δ 11.72 (s, 1H), 9.92 (s, 1H), 8.04 (s, 1H), 7.97 (s, 1H), 1.36 (s, 12H).

### Preparation of 8-[(1R)-1-[(2-bromo-6-chloro-3-pyridyl)amino]ethyl]-2-(4,4-dimethyl-1-piperidyl)-3,6-dimethyl-chromen-4-one

The title compound was prepared as a light yellow solid (1.5 g, 2.89 mmol, 47.48% yield) according to the similar procedure in Example 78, using 8-[(1R)-1-aminoethyl]-2-(4,4-dimethyl-1-piperidyl)-3,6-dimethyl-chromen-4-one (2.00 g, 6.09 mmol, 1 eq) and 2-bromo-6-chloro-3-iodo-pyridine (3.88 g, 12.18 mmol, 2 eq). ¹H NMR (CDCl₃, 400 MHz) δ 7.90 (s, 1H), 7.28-7.27 (m, 1H), 6.97 (d, J=8.4 Hz, 1H), 6.48 (d, J=8.4 Hz, 1H), 4.93-4.90 (m, 1H), 4.82 (d, J=5.2 Hz, 1H), 3.42-3.39 (m, 4H), 2.37 (s, 3H), 2.06 (s, 3H), 1.70 (d, J=6.8 Hz, 3H), 1.55-1.52 (m, 4H), 1.05 (s, 6H).

### Preparation of 3-chloro-5-[6-chloro-3-[[(1R)-1-[2-(4,4-dimethyl-1-piperidyl)-3,6-dimethyl-4-oxo-chromen-8-yl]ethyl]amino]-2-pyridyl]-2-hydroxy-benzaldehyde

The title compound was prepared as light yellow solid (0.7 g, 1.18 mmol, 50.91% yield) according to the procedures described herein and the synthetic scheme, using 8-[(1R)-1-[(2-bromo-6-chloro-3-pyridyl)amino]ethyl]-2-(4,4-dimethyl-1-piperidyl)-3,6-dimethyl-chromen-4-one (1.20 g, 2.31 mmol, 1 eq) and 3-chloro-2-hydroxy-5-(4,4,5,5-tetramethyl-1,3,2-dioxaborolan-2-yl)benzaldehyde (1.70 g, 3.01 mmol, 1.3 eq). ¹H NMR (CDCl₃, 400 MHz) δ 11.62 (s, 1H), 9.97 (s, 1H), 8.04 (d, J=2.0 Hz, 1H), 7.92-7.91 (m, 2H), 7.27-7.26 (m, 1H), 7.03 (d, J=8.8 Hz, 1H), 6.70 (d, J=8.8 Hz, 1H), 4.92-4.90 (m, 1H), 4.76-4.75 (m, 1H), 3.36-3.35 (m, 4H), 2.39 (s, 3H), 2.07 (s, 3H), 1.60 (d, J=6.8 Hz, 3H), 1.52-1.49 (m, 4H), 1.03 (s, 6H).

### Preparation of [2-chloro-4-[6-chloro-3-[[(1R)-1-[2-(4,4-dimethyl-1-piperidyl)-3,6-dimethyl-4-oxo-chromen-8-yl]ethyl]amino]-2-pyridyl]-6-formyl-phenyl]trifluoromethanesulfonate

The title compound was prepared as a light yellow solid (0.76 g, 1.05 mmol, 91.45% yield) according to the procedures described herein and the synthetic scheme, using 3-chloro-5-[6-chloro-3-[[(1R)-1-[2-(4,4-dimethyl-1-piperidyl)-3,6-dimethyl-4-oxo-chromen-8-yl]ethyl]amino]-2-pyridyl]-2-hydroxy-benzaldehyde (0.68 g, 1.14 mmol, 1 eq) and 1,1,1-trifluoro-N-phenyl-N-(trifluoromethylsulfonyl)methanesulfonamide (818 mg, 2.29 mmol, 2 eq). ¹H NMR (CDCl₃, 400 MHz) δ 10.29 (s, 1H), 8.36 (d, J=2.4 Hz, 1H), 8.22 (d, J=2.0 Hz, 1H), 7.93 (s, 1H), 7.30-7.29 (m, 1H), 7.06 (d, J=8.8 Hz, 1H), 6.71 (d, J=8.4 Hz, 1H), 4.96 (d, J=5.2 Hz, 1H), 4.40 (s, 1H), 3.41-3.39 (m, 4H), 2.39 (s, 3H), 2.08 (s, 3H), 1.60 (d, J=6.8 Hz, 3H), 1.54-1.51 (m, 4H), 1.05 (s, 6H).

### Preparation of 3-chloro-5-[6-chloro-3-[[(1R)-1-[2-(4,4-dimethyl-1-piperidyl)-3,6-dimethyl-4-oxo-chromen-8-yl]ethyl]amino]-2-pyridyl]-2-(4,4,5,5-tetramethyl-1,3,2-dioxahorolan-2-yl)benzaldehyde

The title compound was prepared as brown oil (400 mg, crude) according to the procedures described herein and the synthetic scheme, using [2-chloro-4-[6-chloro-3-[[(1R)-1-[2-(4,4-dimethyl-1-piperidyl)-3,6-dimethyl-4-oxo-chromen-8-yl]ethyl]amino]-2-pyridyl]-6-formyl-phenyl]trifluoromethanesulfonate (600 mg, 825.78 μmol, 1 eq) and B₂Pin₂ (839 mg, 3.30 mmol, 4 eq). The product was used directly for next step without further purification.

### Preparation of 8-[(1R)-1-[[6-chloro-2-(8-chloro-1-hydroxy-2,3,1-benzoxazaborinin-6-yl)-3-pyridyl]amino]ethyl]-2-(4,4-dimethyl-1-piperidyl)-3,6-dimethyl-chromen-4-one

The title compound was prepared as a light yellow (25.1 mg, 40.02 μmol, 47.67% yield) according to the procedures described herein and the synthetic scheme, using 3-chloro-5-[6-chloro-3-[[(1R)-1-[2-(4,4-dimethyl-1-piperidyl)-3,6-dimethyl-4-oxo-chromen-8-yl]ethyl]amino]-2-pyridyl]-2-(4,4,5,5-tetramethyl-1,3,2-dioxaborolan-2-yl)benzaldehyde (400 mg, 567.79 μmol, 1 eq) and NH₂OH—HCl (59 mg, 851.68 μmol, 1.5 eq). ¹H NMR (DMSO-d₆, 400 MHz) δ 9.10 (s, 1H), 8.68 (s, 1H), 8.00 (s, 1H), 7.98 (s, 1H), 7.62 (s, 1H), 7.45 (s, 1H), 7.21 (d, J=8.4 Hz, 1H), 6.99 (d, J=8.8 Hz, 1H), 6.07 (d, J=7.2 Hz, 1H), 4.97-4.91 (m, 1H), 3.31-3.28 (m, 4H), 2.32 (s, 3H), 1.89 (s, 3H), 1.50 (d, J=6.8 Hz, 3H), 1.41-1.38 (m, 4H), 0.95 (s, 6H) MS (ESI): mass calcd. For C₃₂H₃₃BCl₂N₄O₄ 618.20, m/z found 619.3 [M+H]⁺. HPLC: 98.75% (220 nm), 99.08% (254 nm). Chiral purity: 99.76% ee.

### Example 71

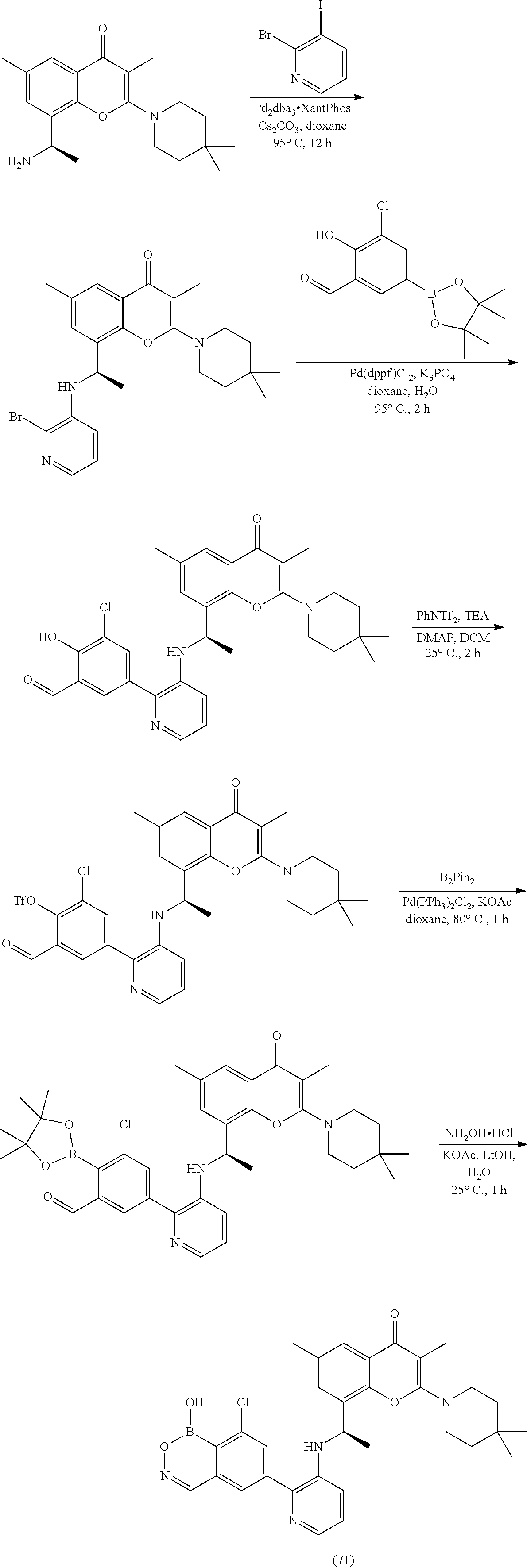

### Preparation of 8-[(1R)-1-[(2-bromo-3-pyridyl)amino]ethyl]-2-(4,4-dimethyl-1-piperidyl)-3,6-dimethyl-chromen-4-one

The title compound was prepared as light yellow oil (1.20 g, 2.48 mmol, 81.36% yield) according to the similar procedure in Example 78, using 8-[(1R)-1-aminoethyl]-2-(4,4-dimethyl-1-piperidyl)-3,6-dimethyl-chromen-4-one (1.00 g, 3.04 mmol, 1 eq) and 2-bromo-3-iodo-pyridine (1.73 g, 6.09 mmol, 2 eq). ¹H NMR (CHCl₃, 400 MHz) δ 7.90 (s, 1H), 7.69 (dd, J=4.4 Hz, J=1.2 Hz, 1H), 7.32 (d, J=2.0 Hz, 1H), 6.95 (q, J=4.4 Hz, 1H), 6.51 (dd, J=8.0 Hz, J=1.2 Hz, 1H), 4.96-4.91 (m, 1H), 4.84 (d, J=4.8 Hz, 1H), 3.42-3.40 (m, 4H), 2.37 (s, 3H), 2.06 (s, 3H), 1.70 (d, J=6.8 Hz, 3H), 1.55-1.52 (m, 4H), 1.05 (s, 6H).

### Preparation of 3-chloro-5-[3-[[(1R)-1-[2-(4,4-dimethyl-1-piperidyl)-3,6-dimethyl-4-oxo-chromen-8-yl]ethyl]amino]-2-pyridyl]-2-hydroxy-benzaldehyde

The title compound was prepared as a light yellow solid (400 mg, 714.18 μmol, 57.66% yield) according to the procedures described herein and the synthetic scheme, using 8-[(1R)-1-[(2-bromo-3-pyridyl)amino]ethyl]-2-(4,4-dimethyl-1-piperidyl)-3,6-dimethyl-chromen-4-one (600 mg, 1.24 mmol, 1 eq) and 3-chloro-2-hydroxy-5-(4,4,5,5-tetramethyl-1,3,2-dioxaborolan-2-yl)benzaldehyde (909.82 mg, 1.61 mmol, 1.3 eq). ¹H NMR (CHCl₃, 400 MHz) δ 11.62 (s, 1H), 9.98 (s, 1H), 8.07-8.06 (m, 2H), 7.94 (d, J=2.0 Hz, 1H), 7.90 (d, J=1.6 Hz, 1H), 7.30 (d, J=2.0 Hz, 1H), 7.06-7.04 (m, 1H), 6.75 (d, J=8.4 Hz, 1H), 4.97-4.94 (m, 1H), 4.38 (d, J=4.4 Hz, 1H), 3.37-3.33 (m, 4H), 2.39 (s, 3H), 2.06 (s, 3H), 1.60 (d, J=6.8 Hz, 3H), 1.52-1.49 (m, 4H), 1.03 (s, 6H).

### Preparation of [2-chloro-4-[3-[[(1R)-1-[2-(4,4-dimethyl-1-piperidyl)-3,6-dimethyl-4-oxo-chromen-8-yl]ethyl]amino]-2-pyridyl]-6-formyl-phenyl]trifluoromethanesulfonate

The title compound was prepared as a light yellow (0.67 g, 968.01 μmol, 80.92% yield) according to the procedures described herein and the synthetic scheme, using 3-chloro-5-[3-[[(1R)-1-[2-(4,4-dimethyl-1-piperidyl)-3,6-dimethyl-4-oxo-chromen-8-yl]ethyl]amino]-2-pyridyl]-2-hydroxy-benzaldehyde (670 mg, 1.20 mmol, 1 eq). ¹H NMR (CHCl₃, 400 MHz) δ 10.29 (s, 1H), 8.38 (d, J=2.0 Hz, 1H), 8.24 (d, J=2.0 Hz, 1H), 8.08 (dd, J=4.8 Hz, J=1.2 Hz, 1H), 7.90 (d, J 1.6 Hz, 1H), 7.33 (d, J=2.4 Hz, 1H), 7.09 (q, J=4.4 Hz, 1H), 6.75 (d, J=8.4 Hz, 1H), 5.01-4.98 (m, 1H), 4.41 (d, J=4.0 Hz, 1H), 3.40-3.38 (m, 4H), 2.39 (s, 3H), 2.07 (s, 3H), 1.62 (d, J=6.4 Hz, 3H), 1.54-1.51 (m, 4H), 1.04 (s, 6H).

### Preparation of 3-chloro-5-[3-[[(1R)-1-[2-(4,4-dimethyl-1-piperidyl)-3,6-dimethyl-4-oxo-chromen-8-yl]ethyl]amino]-2-pyridyl]-2-(4,4,5,5-tetramethyl-1,3,2-dioxaborolan-2-yl)benzaldehyde

The title compound was prepared as a brown oil (0.4 g, crude) according to the procedure described in preceding examples, using [2-chloro-4-[3-[[(1R)-1-[2-(4,4-dimethyl-1-piperidyl)-3,6-dimethyl-4-oxo-chromen-8-yl]ethyl]amino]-2-pyridyl]-6-formyl-phenyl]trifluoromethanesulfonate (600 mg, 866.87 μmol, 1 eq), and B₂Pin₂ (880 mg, 3.47 mmol, 4 eq).

### Preparation of 8-[(1R)-1-[[2-(8-chloro-1-hydroxy-2,3,1-benzoxazaborinin-6-yl)-3-pyridyl]amino]ethyl]-2-(4,4-dimethyl-1-piperidyl)-3,6-dimethyl-chromen-4-one

The title compound was prepared as a white solid (34.3 mg, 58.26 gmol, 48.68% yield) according to the procedures described herein and the synthetic scheme, using 3-chloro-5-[3-[[(1R)-1-[2-(4,4-dimethyl-1-piperidyl)-3,6-dimethyl-4-oxo-chromen-8-yl]ethyl]amino]-2-pyridyl]-2-(4,4,5,5-tetramethyl-1,3,2-dioxaborolan-2-yl)benzaldehyde (400 mg, 596.98 gmol, 1 eq) and NH₂OH—HCl (63 mg, 895.46 μmol, 1.5 eq). ¹H NMR (DMSO-d₆, 400 MHz) δ 9.10 (s, 1H), 8.67 (s, 1H), 8.04 (s, 1H), 8.01 (s, 1H), 7.96 (dd, J=4.8 Hz, J=1.2 Hz, 1H), 7.61 (s, 1H), 7.49 (d, J=1.6 Hz, 1H), 7.14-7.10 (m, 1H), 6.92 (d, J=8.0 Hz, 1H), 5.84 (d, J=7.6 Hz, 1H), 4.98-4.93 (m, 1H), 3.29-2.28 (m, 4H), 2.32 (s, 3H), 1.90 (s, 3H), 1.52 (d, J=6.8 Hz, 3H), 1.42-1.39 (m, 4H), 0.96 (s, 6H). MS (ESI): mass calcd. For C₃₂H₃₄BClN₄O₄ 584.24, m/z found 585.3 [M+H]⁺. HPLC: 99.35% (220 nm), 99.46% (254 nm).

### Example 72

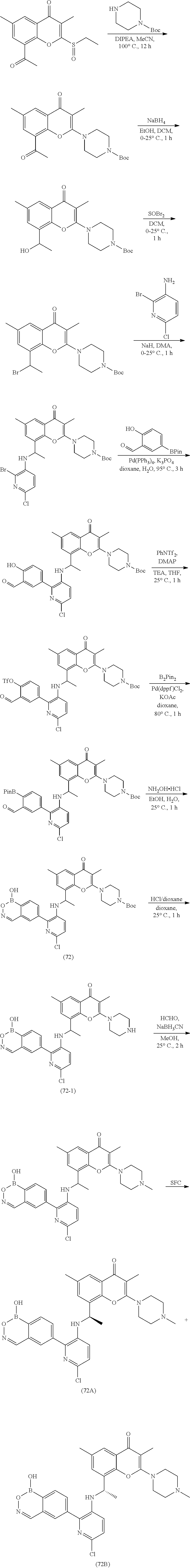

### Preparation of tert-butyl 4-(8-acetyl-3,6-dimethyl-4-oxo-chromen-2-yl)piperazine-1-carboxylate

To a solution of 8-acetyl-2-ethylsulfinyl-3,6-dimethyl-chromen-4-one (20.00 g, 68.41 mmol, 1 eq) and tert-butyl piperazine-1-carboxylate (21.66 g, 116.30 mmol, 1.7 eq) in MeCN (400 mL) was added DIEA (26.52 g, 205.23 mmol, 35.75 mL, 3 eq) dropwise at 25° C. The reaction mixture was directly moved to the oil-bath (pre-heated to 100° C.) and stirred at 100° C. for 12 h. The reaction mixture was cooled to 25° C., filtered and the filter cake was dried in vacuo to give the title compound (10.00 g, 24.97 mmol, 36.50% yield) as a yellow solid. ¹H NMR (CDCl₃, 400 MHz) δ 8.19 (s, 1H), 7.81 (d, J=2 Hz, 1H), 3.61-3.59 (m, 4H), 3.45-3.43 (m, 4H), 2.70 (s, 3H), 2.47 (s, 3H), 2.07 (s, 3H), 1.50 (s, 9H).

### Preparation of tert-butyl 4-[8-(1-hydroxyethyl)-3,6-dimethyl-4-oxo-chromen-2-yl]piperazine-1-carboxylate

To a solution of tert-butyl 4-(8-acetyl-3,6-dimethyl-4-oxo-chromen-2-yl)piperazine-1-carboxylate (7.00 g, 17.48 mmol, 1 eq) in EtOH (40 mL)/DCM (40 mL) was added NaBH₄ (1.52 g, 40.18 mmol, 2.30 eq) in portions at 0° C. The reaction mixture was stirred at 25° C. for 1 h. The reaction mixture was quenched by addition ice water (100 mL) at 0° C., and then extracted with EtOAc (80 mL×3). The combined organic layers were washed with brine (80 mL), dried over Na₂SO₄, filtered and concentrated under reduced pressure to give the title compound (6.80 g, 16.90 mmol, 96.66% yield) as a white solid. ¹H NMR (CDCl₃, 400 MHz) δ 7.72 (d, J=1.2 Hz, 1H), 7.49 (d, J=2 Hz, 1H), 5.32-5.30 (m, 1H), 3.58 (t, J=4.4 Hz, 4H), 3.35 (t, J=5.2 Hz, 4H), 2.36 (s, 3H), 2.00 (s, 3H), 1.54 (d, J=6.4 Hz, 3H), 1.49 (s, 9H).

### Preparation of tert-butyl 4-[8-(1-bromoethyl)-3,6-dimethyl-4-oxo-chromen-2-yl]piperazine-1-carboxylate

To a solution of tert-butyl 4-[8-(1-hydroxyethyl)-3,6-dimethyl-4-oxo-chromen-2-yl]piperazine-1-carboxylate (3.00 g, 7.45 mmol, 1 eq) and sulfurous dibromide (2.01 g, 9.69 mmol, 750.75 μL, 1.3 eq) in DCM (30 mL) was added DMF (54.48 mg, 745.37 μmol, 57.35 μL, 0.1 eq) dropwise at 0° C. The reaction mixture was warmed to 25° C. and stirred at 25° C. for 1 h. 2 parallel reactions were combined for work up. The reaction mixture was quenched by adding water (60 mL) at 0° C., and then extracted with DCM (50 mL×3). The combined organic layers were washed with NaHCO₃ (60 mL×2), dried over Na₂SO₄, filtered and concentrated under reduced pressure to give the title compound (6.80 g, 14.61 mmol, 98.02% yield) as a yellow solid. ¹H NMR (CDCl₃, 400 MHz) δ 7.94 (d, J=1.2 Hz, 1H), 7.53 (d, J=2 Hz, 1H), 5.64 (q, J=7.2 Hz, 6.8 Hz, 1H), 3.62 (t, J=4.4 Hz, 4H), 3.44 (t, J=5.2 Hz, 4H), 2.44 (s, 3H), 2.15 (d, J=7.2 Hz, 3H), 2.05 (s, 3H), 1.50 (s, 9H).

### Preparation of tert-butyl 4-[8-[1-[(2-bromo-6-chloro-3-pyridyl)amino]ethyl]-3,6-dimethyl-4-oxo-chromen-2-yl]piperazine-1-carboxylate

The title compound was prepared as a yellow solid (5.00 g, 8.45 mmol, 67.78% yield) according to the procedures described herein and the synthetic scheme, using 2-bromo-6-chloro-pyridin-3-amine (3.10 g, 14.96 mmol, 1.2 eq) and tert-butyl 4-[8-(1-bromoethyl)-3,6-dimethyl-4-oxo-chromen-2-yl]piperazine-1-carboxylate (5.80 g, 12.46 mmol, 1 eq). ¹H NMR (DMSO-d₆, 400 MHz) δ 7.88 (d, J=1.6 Hz, 1H), 7.29 (d, J=2.0 Hz, 1H), 6.94 (d, J=8.4 Hz, 1H), 6.43 (d, J=8.4 Hz, 1H), 4.92-4.85 (m, 1H), 4.79 (d, J=5.2 Hz, 1H), 3.60 (d, J=4.8 Hz, 4H), 3.36 (d, J=4.8 Hz, 4H), 2.36 (s, 3H), 2.05 (s, 3H), 1.68 (d, J=6.8 Hz, 3H), 1.48 (s, 9H).

### Preparation of tert-butyl 4-[8-[1-[[6-chloro-2-(3-formyl-4-hydroxy-phenyl)-3-pyridyl]amino]ethyl]-3,6-dimethyl-4-oxo-chromen-2-yl]piperazine-1-carboxylate

The title compound was prepared as a yellow oil (1.50 g, 2.37 mmol, 70.12% yield) according to the procedures described herein and the synthetic scheme, using tert-butyl 4-[8-[1-[(2-bromo-6-chloro-3-pyridyl)amino]ethyl]-3,6-dimethyl-4-oxo-chromen-2-yl]piperazine-1-carboxylate (2.00 g, 3.38 mmol, 1 eq) and 2-hydroxy-5-(4,4,5,5-tetramethyl-1,3,2-dioxaborolan-2-yl)benzaldehyde (1.01 g, 4.05 mmol, 1.2 eq). ¹H NMR (DMSO-d₆, 400 MHz) δ 11.12 (s, 1H), 9.98 (s, 2H), 7.96 (d, J=2.4 Hz, 1H), 7.92, (dd, J=2.0 Hz, 2.4 Hz, 1H), 7.89 (d, J=2.0 Hz, 1H), 7.29 (d, J=2.0 Hz, 1H), 7.15 (d, J=8.8 Hz, 1H), 6.98 (d, J=8.8 Hz, 1H), 6.59 (d, J=8.4 Hz, 1H), 4.91-4.84 (m, 1H), 4.39 (d, J=5.2 Hz, 1H), 3.59 (t, J=5.2 Hz, 4H), 3.31 (t, J=4.4 Hz, 4H), 2.37 (s, 3H), 2.06 (s, 3H), 1.55 (d, J=6.8 Hz, 3H), 1.49 (s, 9H).

### Preparation of tert-butyl 4-[8-[1-[[6-chloro-2-[3-formyl-4-(trifluoromethylsulfonyloxy) phenyl]-3-pyridyl]amino]ethyl]-3,6-dimethyl-4-oxo-chromen-2-yl]piperazine-1-carboxylate

The title compound was prepared as a yellow solid (1.50 g, 1.96 mmol, 82.74% yield) according to the procedures described herein and the synthetic scheme, using tert-butyl-4-[8-[1-[[6-chloro-2-(3-formyl-4-hydroxy-phenyl)-3-pyridyl]amino]ethyl]-3,6-dimethyl-4-oxo-chromen-2-yl]piperazine-1-carboxylate (1.50 g, 2.37 mmol, 1 eq) and PhNTf₂ (846 mg, 2.37 mmol, 1 eq). ¹H NMR (CDCl₃, 400 MHz) δ 10.34 (s, 1H), 8.43 (d, J=2.4 Hz, 1H), 8.13 (dd, J=2.0 Hz, 2.4 Hz, 1H), 7.90 (d, J=1.6 Hz, 1H), 7.59 (d, J=8.4 Hz, 1H), 7.31 (d, J=2.0 Hz, 1H), 7.03 (d, J=8.8 Hz, 1H), 6.63 (d, J=8.8 Hz, 1H), 4.98-4.86 (m, 1H), 4.37 (d, J=4.4 Hz, 1H), 3.60 (t, J=4.4 Hz, 4H), 3.36 (t, J=5.2 Hz, 4H), 2.38 (s, 3H), 2.07 (s, 3H), 1.57 (d, J=6.8 Hz, 3H), 1.49 (s, 9H).

### Preparation of tert-butyl 4-[8-[1-[[6-chloro-2-[3-formyl-4-(4,4,5,5-tetramethyl-1,3,2-dioxaborolan-2-yl)phenyl]-3-pyridyl]amino]ethyl]-3,6-dimethyl-4-oxo-chromen-2-yl]piperazine-1-carboxylate

The title compound was prepared as a yellow solid (1.30 g, crude) according to the procedures described herein and the synthetic scheme, using tert-butyl 4-[8-[1-[[6-chloro-2-[3-formyl-4-(trifluoromethylsulfonyloxy) phenyl]-3-pyridyl]amino]ethyl]-3,6-dimethyl-4-oxo-chromen-2-yl]piperazine-1-carboxylate (1.50 g, 1.96 mmol, 1 eq) and B₂Pin₂ (1.99 g, 7.84 mmol, 4 eq). The product was used directly for next step without further purification.

### Preparation of tert-butyl 4-[8-[1-[[6-chloro-2-(1-hydroxy-2,3,1-benzoxazaborinin-6-yl)-3-pyridyl]amino]ethyl]-3,6-dimethyl-4-oxo-chromen-2-yl]piperazine-1-carboxylate

The title compound was prepared as a white solid (410 mg, 623 μmol, 77.18% yield) according to the procedures described herein and the synthetic scheme, using tert-butyl 4-[8-[1-[[6-chloro-2-[3-formyl-4-(4,4,5,5-tetramethyl-1,3,2-dioxaborolan-2-yl)phenyl]-3-pyridyl]amino]ethyl]-3,6-dimethyl-4-oxo-chromen-2-yl]piperazine-1-carboxylate (600 mg, 807 μmol, 1 eq) and NH₂OH·HCl (112.22 mg, 1.61 mmol, 2 eq). ¹H NMR (DMSO-d₆, 400 MHz) δ 8.69 (s, 1H), 8.18-8.13 (m, 1H), 8.07-7.82 (m, 2H), 7.63 (s, 2H), 7.44 (d, J=2 Hz, 1H), 7.16 (d, J=8.8 Hz, 1H), 6.93 (d, J=8.8 Hz, 1H), 5.83 (s, 1H), 4.95 (t, J=6.4 Hz, 1H), 3.47 (m, 4H), 3.20-3.15 (m, 4H), 2.31 (s, 3H), 1.92 (s, 3H), 1.49 (d, J=6.8 Hz, 3H), 1.42 (s, 9H). MS (ESI): mass calcd. For C₃₄H₃₇BClN₅O₆ 657.25, m/z found 658.1 [M+H]⁺. HPLC: 97.19% (220 nm), 98.14% (254 nm).

### Preparation of 8-[1-[[6-chloro-2-(1-hydroxy-2,3,1-benzoxazaborinin-6-yl)-3-pyridyl]amino]ethyl]-3,6-dimethyl-2-piperazin-1-yl-chromen-4-one

To a solution of tert-butyl 4-[8-[1-[[6-chloro-2-(1-hydroxy-2,3,1-benzoxazaborinin-6-yl)-3-pyridyl]amino]ethyl]-3,6-dimethyl-4-oxo-chromen-2-yl]piperazine-1-carboxylate (250 mg, 379 μmol, 1 eq) in dioxane (5 mL) was added dropwise HCl/dioxane (4 N, 284 μL, 3 eq) at 25° C., the reaction mixture was stirred at 25° C. for 1 h. The reaction mixture was filtered and the filter cake was dried in vacuo to give a residue. The residue was purified by prep-HPLC (column: Waters Xbridge Prep OBD C18 150×40 mm×10 um; mobile phase: [H₂O (10 mM NH₄HCO₃)-AC□]; gradient: 15%-45% B over 8.0 min) to give the title compound (5 mg, 9.0 μmol, 2.36% yield) as a white solid. ¹H NMR (DMSO-d₆, 400 MHz) δ 8.65 (s, 1H), 8.12 (d, J=8.4 Hz, 1H), 8.00 (s, 2H), 7.61 (s, 1H), 7.44 (d, J=1.2 Hz, 1H), 7.19 (d, J=8.4 Hz, 1H), 7.00 (d, J=8.4 Hz, 1H), 5.83 (d, J=6.8 Hz, 1H), 4.91-4.82 (m, 1H), 3.24-3.22 (m, 4H), 2.76 (t, J=8.8 Hz, 4H), 2.31 (s, 3H), 1.88 (s, 3H), 1.48 (d, J=6.8 Hz, 3H). MS (ESI): mass calcd. For C₂₉H₂₉BClN₅O₄557.20, m/z found 558.0 [M+H]⁺. HPLC: 96.74% (220 nm), 96.96% (254 nm).

### Preparation of 8-[1-[[6-chloro-2-(1-hydroxy-2,3,1-benzoxazaborinin-6-yl)-3-pyridyl]a mino]ethyl]-3,6-dimethyl-2-(4-methylpiperazin-1-yl)chromen-4-one

To a solution of 8-[1-[[6-chloro-2-(1-hydroxy-2,3,1-benzoxazaborinin-6-yl)-3-pyridyl]amino]ethyl]-3,6-dimethyl-2-piperazin-1-yl-chromen-4-one (350 mg, 627.43 μmol, 1 eq) and formaldehyde (153 mg, 1.88 mmol, 140 μL, 3 eq) in MeOH (4 mL) was added NaBH₃CN (79 mg, 1.25 mmol, 2 eq) in portions at 25° C. The reaction mixture was stirred at 25° C. for 2 h. The reaction mixture was quenched by adding water (10 mL) at 25° C., and then extracted with EtOAc (10 mL×3). The combined organic layers were washed with brine (20 mL), dried over Na₂SO₄, filtered and concentrated under reduced pressure to give a residue. The residue was purified by prep-HPLC (column: Waters Xbridge BEH C18 100×30 mm×10 um; mobile phase: [H₂O (10 mM NH₄HCO₃)-AC□]; gradient: 10%-45% B over 8.0 min) to give the title compound (40 mg, 69.95 μmol, 11.15% yield) as a white solid.

### Preparation of 8-[(1S)-1-[[6-chloro-2-(1-hydroxy-2,3,1-benzoxazaborinin-6-yl)-3-pyridyl]amino]ethyl]-3,6-dimethyl-2-(4-methylpiperazin-1-yl)chromen-4-one and 8-[(1R)-1-[[6-chloro-2-(1-hydroxy-2,3,1-benzoxazaborinin-6-yl)-3-pyridyl]amino]ethyl]-3,6-dimethyl-2-(4-methylpiperazin-1-yl)chromen-4-one

8-[1-[[6-chloro-2-(1-hydroxy-2,3,1-benzoxazaborinin-6-yl)-3-pyridyl]amino]ethyl]-3,6-dimethyl-2-(4-methylpiperazin-1-yl)chromen-4-one (0.02 g. 34.97 μmol, 1 eq) was separated by SFC (column: ChiralPak IH, 250×30 mm×10 um; mobile phase: [CO₂—IPA]; B %: 40%, isocratic elution mode) to give the title compound, Isomer 1 (5 mg, 8.74 μmol, 25.00% yield) as a white solid and the title compound, Isomer 2 (5 mg, 8.74 μmol, 25.00% yield) as a white solid. Isomer 1: ¹H NMR (DMSO-d₆, 400 MHz) δ 9.42 (s, 1H), 8.71 (s, 1H), 8.16 (d, J=7.6 Hz, 1H), 8.05 (d, J=8.8 Hz, 2H), 7.62 (d, J=1.2 Hz, 1H), 7.48 (d, J=1.6 Hz, 1H), 7.19 (d, J=8.4 Hz, 1H), 6.99 (d, J=8.8 Hz, 1H), 5.79 (d, J=7.2 Hz, 1H), 5.02-4.81 (m, 1H), 3.27-3.19 (m, 4H), 2.37 (t, J=4.8 Hz, 4H), 2.32 (s, 3H), 2.20 (s, 3H), 1.88 (s, 3H), 1.50 (d, J=6.8 Hz, 3H). MS (ESI): mass calcd. For C₃₀H₃₁BClN₅O₄ 571.22, m/z found 572.2[M+H]⁺. HPLC: 94.85% (220 nm), 94.46% (254 nm). Chiral purity: 96.88% ee. Isomer 2: ¹H NMR (DMSO-d₆, 400 MHz) δ 9.44 (s, 1H), 8.71 (s, 1H), 8.17 (d, J=7.6 Hz, 1H), 8.05 (d, J=8.0 Hz, 2H), 7.62 (d, J 1.2 Hz, 1H), 7.48 (d, J=2.0 Hz, 1H), 7.19 (d, J=8.4 Hz, 1H), 6.99 (d, J=8.8 Hz, 1H), 5.79 (d, J=7.2 Hz, 1H), 4.96-4.89 (m, 1H), 3.26-3.20 (m, 4H), 2.37 (t, J=4.8 Hz, 4H), 2.32 (s, 3H), 2.20 (s, 3H), 1.88 (s, 3H), 1.50 (d, J=6.8 Hz, 3H). MS (ESI): mass calcd. For C₃₀H₃₁BClN₅O₄571.22, m/z found 572.2[M+H]⁺. HPLC: 96.98% (220 nm), 98.01% (254 nm). Chiral purity: 100% ee

### Example 73

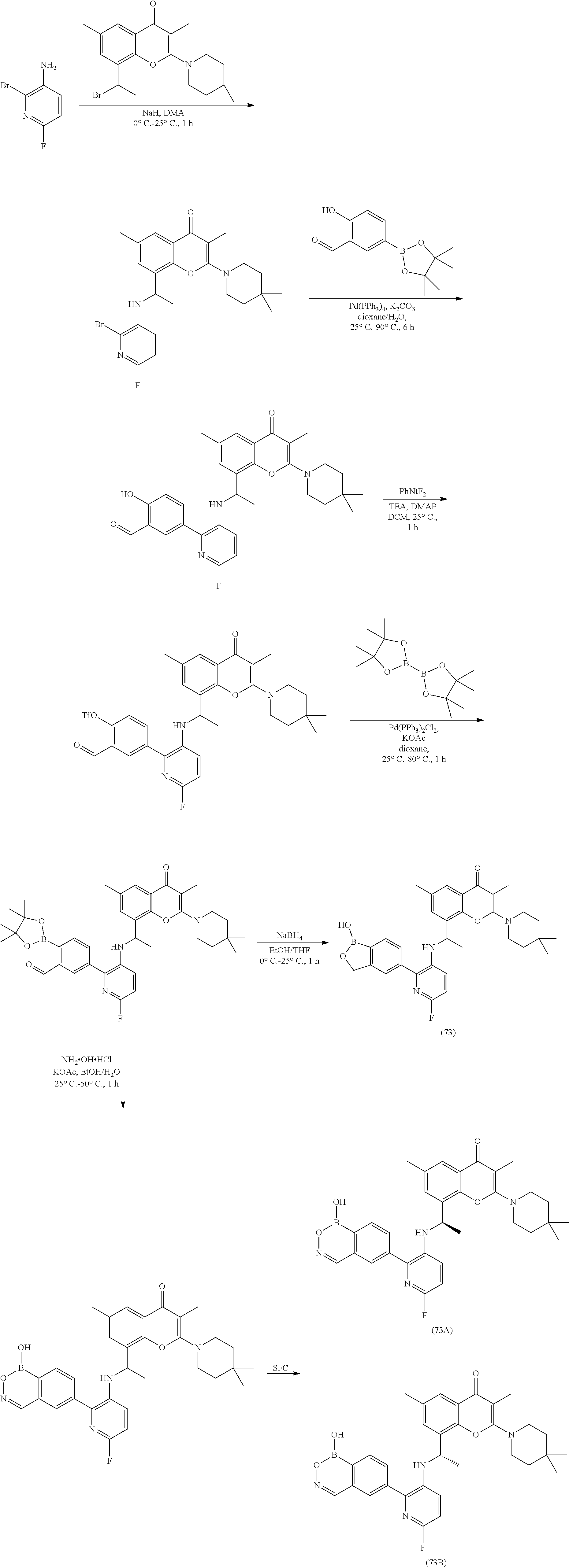

### Preparation of 8-[1-[(2-bromo-6-fluoro-3-pyridyl)amino]ethyl]-2-(4,4-dimethyl-1-piperidyl)-3,6-dimethyl-chromen-4-one

To a solution of 2-bromo-6-fluoro-pyridin-3-anine (876 mg, 4.59 mmol, 1.2 eq) in DMA (10 mL) was added NaH (230 mg, 5.73 mmol, 1.5 eq) in portions at 0° C., the reaction mixture was warmed to 25° C. and stirred for 15 min. Then the reaction mixture was cooled to 0° C. To the above reaction mixture was added dropwise a solution of 8-(1-bromoethyl)-2-(4,4-dimethyl-1-piperidyl)-3,6-dimethyl-chromen-4-one (1.50 g, 3.82 mmol, 1 eq) in DMA (10 mL) at 0° C., the resulting reaction mixture was warmed to 25° C. and stirred for 45 min. The reaction mixture was poured into H₂O (100 mL) at 0° C. and then extracted with EtOAc (60 mL×3). The combined organic phases were washed with brine (100 mL×2), dried over Na₂SO₄, filtered and concentrated under reduced pressure to give a residue. The residue was purified by flash silica gel chromatography (Biotage®; 40 g SepaFlash® Silica Flash Column, Eluent of 0~30% Ethyl acetate/Petroleum ether gradient @80 mL/min) to give the title compound (1.20 g, 2.39 mmol, 62.47% yield) as a green solid. ¹H NMR (CDCl₃, 400 MHz) δ 7.91 (s, 1H), 7.31 (d, J=2.0 Hz, 1H), 6.66-6.59 (m, 2H), 4.96-4.83 (m, 1H), 4.67 (s, 1H), 3.44-3.36 (m, 4H), 2.38 (s, 3H), 2.07 (s, 3H), 1.69 (d, J=6.8 Hz, 3H), 1.56-1.51 (m, 4H), 1.05 (s, 6H).

### Preparation of 5-[3-[1-[2-(4,4-dimethyl-1-piperidyl)-3,6-dimethyl-4-oxo-chromen-8-yl]ethylamino]-6-fluoro-2-pyridyl]-2-hydroxy-benzaldehyde

To a mixture of 8-[1-[(2-bromo-6-fluoro-3-pyridyl)amino]ethyl]-2-(4,4-dimethyl-1-piperidyl)-3,6-dimethyl-chromen-4-one (700 mg, 1.39 mmol, 1 eq) and 2-hydroxy-5-(4,4,5,5-tetramethyl-1,3,2-dioxaborolan-2-yl)benzaldehyde (518 mg, 2.09 mmol, 1.5 eq) in dioxane (10 mL)/H₂O (2 mL) were added K₂CO₃ (481 mg, 3.48 mmol, 2.5 eq) and Pd(PPh₃)₄ (322 mg, 279 μmol, 0.2 eq) in turns at 25° C. The reaction mixture was bubbled N₂ for 1 min and then directly stirred at 90° C. (the oil bath was pre-heated to 90° C.) for 6 h under N₂. The reaction mixture was cooled to 25° C., filtered and the filtrate was concentrated under reduced pressure to give a residue. The residue was purified by flash silica gel chromatography (Biotage®; 12 g SepaFlash® Silica Flash Column, Eluent of 0~40% Ethyl acetate/Petroleum ether gradient @50 mL/min) to give the title compound (700 mg, 1.29 mmol, 92.42% yield) as a yellow solid. ¹H NMR (CDCl₃, 400 MHz) δ 11.17 (s, 1H), 9.98 (s, 1H), 8.03-7.87 (m, 3H), 7.30 (d, J=2.0 Hz, 1H), 7.15 (d, J=8.4 Hz, 1H), 6.80 (dd, J=8.4, 2.0 Hz, 1H), 6.65 (dd, J=8.8, 3.2 Hz, 1H), 4.94-4.82 (m, 1H), 4.52-4.17 (m, 1H), 3.41-3.27 (m, 4H), 2.38 (s, 3H), 2.07 (s, 3H), 1.57 (d, J=6.4 Hz, 3H), 1.50 (t, J=5.6 Hz, 4H), 1.03 (s, 6H).

### Preparation of [4-[3-[1-[2-(4,4-dimethyl-1-piperidyl)-3,6-dimethyl-4-oxo-chromen-8-yl]ethylamino]-6-fluoro-2-pyridyl]-2-formyl-phenyl]trifluoromethanesulfonate

To a solution of 5-[3-[1-[2-(4,4-dimethyl-1-piperidyl)-3,6-dimethyl-4-oxo-chromen-8-yl]ethylamino]-6-fluoro-2-pyridyl]-2-hydroxy-benzaldehyde (700 mg, 1.29 mmol, 1 eq), TEA (2.58 mmol, 358 μL, 2 eq) and DMAP (32 mg, 257 μmol, 0.2 eq) in DCM (15 mL) was added 1,1,1-trifluoro-N-phenyl-N-(trifluoromethylsulfonyl)methanesulfonamide (460 mg, 1.29 mmol, 1 eq) in portions at 25° C., the reaction mixture was stirred at 25° C. for 1 h. The reaction mixture was poured into H₂O (20 mL) and extracted with DCM (15 mL×3). The combined organic layers were washed with brine (30 mL), dried over Na₂SO₄, filtered and concentrated under reduced pressure to give a residue. The residue was purified by flash silica gel chromatography (Biotage®; 12 g SepaFlash® Silica Flash Column, Eluent of 0-40% Ethyl acetate/Petroleum ether gradient @40 m/min) to give the title compound (700 mg, 1.04 mmol, 80.46% yield) as a yellow solid ¹H NMR (CDCl₃, 400 MHz) δ 10.35 (s, 1H), 8.49 (d, J=2.4 Hz, 1H), 8.17 (dd, J=8.8, 2.4 Hz, 1H), 7.93 (s, 1H), 7.69 (d, J=8.8 Hz, 1H), 7.33 (d, J=2.0 Hz, 1H), 6.83 (dd, J=8.8, 2.4 Hz, 1H), 6.71 (dd, J=8.8, 3.6 Hz, 1H), 4.97-4.88 (m, 1H), 4.37-4.20 (m, 1H), 3.41-3.38 (m, 4H), 2.39 (s, 3H), 2.09 (s, 3H), 1.59 (d, J=6.4 Hz, 3H), 1.54-1.51 (m, 4H), 1.04 (s, 6H).

### Preparation of 5-[3-[1-[2-(4,4-dimethyl-1-piperidyl)-3,6-dimethyl-4-oxo-chromen-8-yl]ethylamino]-6-fluoro-2-pyridyl]-2-(4,4,5,5-tetramethyl-1,3,2-dioxaborolan-2-yl)benzaldehyde

To a mixture of [4-[3-[1-[2-(4,4-dimethyl-1-piperidyl)-3,6-dimethyl-4-oxo-chromen-8-yl]ethylamino]-6-fluoro-2-pyridyl]-2-formyl-phenyl]trifluoromethanesulfonate (500 mg, 740 μm ol, 1 eq) and 4,4,5,5-tetramethyl-2-(4,4,5,5-tetramethyl-1,3,2-dioxaborolan-2-yl)-1,3,2-dioxaborolane (752 mg, 2.96 mmol, 4 eq) in dioxane (10 mL) were added Pd(PPh₃)₂Cl₂ (52 mg, 74.0 μmol, 0.1 eq) and KOAc (145 mg, 1.48 mmol, 2 eq) in turns at 25° C. The reaction mixture was bubbled N₂ for 1 min and directly stirred at 80° C. (the oil bath was pre-heated to 80° C.) for 1 h under N₂. The reaction mixture was cooled to 25° C., filtered and the filtrate was concentrated under reduced pressure to give a residue. The residue was purified by flash silica gel chromatography (Biotage®; 12 g SepaFlash® Silica Flash Column, Eluent of 0~30% Ethyl acetate/Petroleum ether gradient @40 mL/min) to give the title compound (350 mg, 535.51 mol, 72.37% yield) as a yellow solid. ¹H NMR (CDCl₃, 400 MHz) δ 10.62 (s, 1H), 8.38 (s, 1H), 8.04 (d, J=7.6 Hz, 1H), 7.98 (dd, J=8.0, 2.0 Hz, 1H), 7.94 (s, 1H), 7.32 (s, 1H), 6.83-6.76 (m, 1H), 6.68 (dd, J=8.4, 3.2 Hz, 1H), 4.95-4.75 (m, 1H), 4.55-4.05 (m, 1H), 3.43-3.34 (m, 4H), 2.39 (s, 3H), 2.10 (s, 3H), 1.53 (d, J=6.8 Hz, 3H), 1.52-1.49 (m, 4H), 1.44 (s, 12H), 1.03 (s, 6H).

### Preparation of 2-(4,4-dimethyl-1-piperidyl)-8-[(1S)-1-[[6-fluoro-2-(1-hydroxy-2,3,1-benzoxazaborinin-6-yl)-3-pyridyl]amino]ethyl]-3,6-dimethyl-chromen-4-one and 2-(4,4-dimethyl-1-piperidyl)-8-[(1R)-1-[[6-fluoro-2-(1-hydroxy-2,3,1-benzoxazaborinin-6-yl)-3-pyridyl]amino]ethyl]-3,6-dimethyl-chromen-4-one

To a solution of 5-[3-[1-[2-(4,4-dimethyl-1-piperidyl)-3,6-dimethyl-4-oxo-chromen-8-yl]ethylamino]-6-fluoro-2-pyridyl]-2-(4,4,5,5-tetramethyl-1,3,2-dioxaborolan-2-yl)benzaldehyde (550 mg, 841 gmol, 1 eq) in EtOH (3 mL)/H₂O (3 mL) were added NH₂OH·HCl (59 mg, 841 μmol, 1 eq) and KOAc (83 mg, 841 μmol, 1 eq) in turns at 25° C. The reaction mixture was bubbled N₂ for 1 min and then the reaction mixture was directly stirred at 50° C. (the oil bath was pre-heated to 50° C.) for 1 h under N₂. The reaction mixture was cooled to 25° C. and the resulting suspension was directly filtered. The filter cake was collected and dried in vacuo. The solid was triturated with acetonitrile (6 mL)/H₂O (2 mL) at 25° C. for 10 min, filtered and dried in vacuo to give 2-(4,4-dimethyl-1-piperidyl)-8-[1-[[6-fluoro-2-(1-hydroxy-2,3,1-benzo xazaborinin-6-yl)-3-pyridyl]amino]ethyl]-3,6-dimethyl-chromen-4-one (350 mg, 584.93 μmol, 69.51% yield) as a yellow solid. The product was further separated by SFC (column: ChiralPak IH, 250×30 mm×10 um; mobile phase: [CO₂—IPA]; B %:40%, isocratic elution mode) to give the title compound, Isomer 1 (118.5 mg, 205.34 gmol, 24.40% yield) as a pale-yellow solid and the title compound, Isomer 2 (130.1 mg, 223.38 μmol, 26.54% yield) as a pale-yellow solid. Isomer 1: ¹H NMR (DMSO-d₆, 400 MHz) δ 9.46 (s, 1H), 8.68 (s, 1H), 8.15 (d, J=7.6 Hz, 1H), 8.12-8.08 (m, 2H), 7.61 (s, 1H), 7.49 (d, J=2.0 Hz, 1H), 7.16 (t, J=8.4 Hz, 1H), 7.93 (dd, J=8.8, 4.0 Hz, 1H), 5.67 (d, J=7.2 Hz, 1H), 4.93-4.81 (m, 1H), 3.26-3.23 (m, 4H), 2.32 (s, 3H), 1.89 (s, 3H), 1.49 (d, J=6.8 Hz, 3H), 1.41-1.33 (m, 4H), 0.95 (s, 6H). MS (ESI): mass calcd. For C₃₂H₃₄BFN₄O₄ 568.27, m/z found 569.3 [M+H]⁺. HPLC: 98.50 (220 nm), 98.64 (254 nm). Chiral purity: 100% ee. Isomer 2: ¹H NMR (DMSO-d₆, 400 MHz) δ 9.46 (s, 1H), 8.68 (s, 1H), 8.15 (d, J=7.6 Hz, 1H), 8.12-8.08 (m, 2H), 7.61 (s, 1H), 7.49 (d, J=2.0 Hz, 1H), 7.16 (t, J=8.4 Hz, 1H), 7.93 (dd, J=8.8, 4.0 Hz, 1H), 5.67 (d, J=7.6 Hz, 1H), 4.93-4.80 (m, 1H), 3.26-3.23 (m, 4H), 2.32 (s, 3H), 1.89 (s, 3H), 1.49 (d, J=6.8 Hz, 3H), 1.4-1.34 (m, 4H), 0.95 (s, 6H). MS (ESI): mass calcd. For C₃₂H₃₄BFN₄O₄ 568.27, m/z found 569.3 [M+H]⁺. HPLC: 97.65 (220 nm), 97.80 (254 nm). Chiral purity: 98.40% ee

### Preparation of 2-(4,4-dimethyl-1-piperidyl)-8-[1-[[6-fluoro-2-(1-hydroxy-3H-2,1-benzoxaborol-5-yl)-3-pyridyl]amino]ethyl]-3,6-dimethyl-chromen-4-one

To a solution of 5-[3-[1-[2-(4,4-dimethyl-1-piperidyl)-3,6-dimethyl-4-oxo-chromen-8-yl]ethylamino]-6-fluoro-2-pyridyl]-2-(4,4,5,5-tetramethyl-1,3,2-dioxaborolan-2-yl)benzaldehyde (100 mg, 153 μmol, 1 eq) in THF (4 mL)/EtOH (0.1 mL) was added NaBH₄ (60 mg, 1.59 mmol, 10.4 eq) in portions at 0° C., the reaction mixture was stirred at 25° C. for 1 h. The reaction mixture was poured into H₂O (6 mL) at 0° C., adjusted pH to 6 with 2N HCl, and then extracted with EtOAc (8 mL×3). The combined organic layers were washed with brine (10 mL×3), dried over Na₂SO₄, filtered and concentrated under reduced pressure to give a residue. The residue was purified by prep-HPLC (column: Waters Xbridge Prep OBD C18 150×40 mm×10 um; mobile phase: [H₂O (10 mM NH₄HCO₃)-AC□]; gradient:50%-80% B over 8.0 min) to give the title compound (31.7 mg, 56.74 μmol, 37.08% yield) as an off white solid. ¹H NMR (DMSO-d₆, 400 MHz) δ 9.24 (s, 1H), 7.82 (d, J=8.0 Hz, 1H), 7.71-7.65 (m, 2H), 7.61 (s, 1H), 7.46 (d, J=2.0 Hz, 1H), 7.12 (t, J=8.4 Hz, 1H), 6.88 (dd, J=8.8, 3.6 Hz, 1H), 5.35 (d, J=7.6 Hz, 1H), 5.06-4.94 (m, 2H), 4.93-4.84 (m, 1H), 3.27-3.23 (m, 4H), 2.32 (s, 3H), 1.89 (s, 3H), 1.49 (d, J=6.8 Hz, 3H), 1.38 (t, J=5.2 Hz, 4H), 0.96 (s, 6H). MS (ESI): mass calcd. For C₃₂H₃₅BFN₃O₄ 555.27, m/z found 556.2 [M+H]⁺. HPLC: 99.42 (220 urn), 99.48 (254 nm).

### Example 74

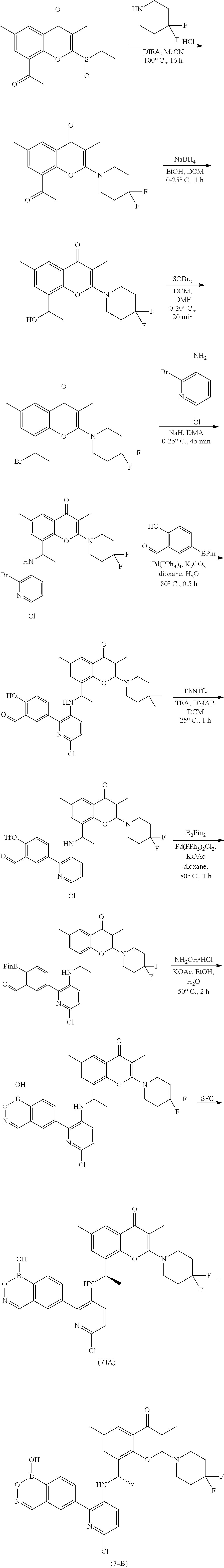

### Preparation of 8-acetyl-2-(4,4-difluoro-1-piperidyl)-3,6-dimethyl-chromen-4-one

To a solution of 8-acetyl-2-ethylsulfinyl-3,6-dimethyl-chromen-4-one (5.0 g, 17.1 mmol, 1 eq) in MeCN (100 mL) were added DIEA (17.6 g, 136 mmol, 23.8 mL, 8 eq) and 4,4-difluoropiperidine;hydrochloride (10.7 g, 68.4 mmol, 4 eq) in turns at 25° C., and then the reaction mixture was directly stirred at 100° C. (the oil-bath was pre-heated to 100° C.) for 16 h. The reaction was cooled to 0° C. and the resulting suspension was directly filtered. The filter cake was further washed with MTBE (10 mL) to give the title compound (2.0 g, 5.96 mmol, 34.8% yield) as a yellow solid. ¹H NMR (DMSO-d₆, 400 MHz) δ 7.99 (d, J=1.6 Hz, 1H), 7.93 (d, J=2.0 Hz, 1H), 3.57-3.51 (m, 4H), 2.70 (s, 3H), 2.44 (s, 3H), 2.21-2.07 (m, 4H), 1.95 (s, 3H).

### Preparation of 2-(4,4-difluoro-1-piperidyl)-8-(1-hydroxyethyl)-3,6-dimethyl-chromen-4-one

To a solution of 8-acetyl-2-(4,4-difluoro-1-piperidyl)-3,6-dimethyl-chromen-4-one (2.00 g, 5.96 mmol, 1 eq) in EtOH (10 mL)/DCM (10 mL) was added NaBH₄ (496 mg, 13.1 mmol, 2.2 eq) in portions at 0° C., the reaction mixture was stirred at 25° C. for 1 h. The reaction mixture was poured into ice water (50 mL) at 0° C. and then extracted with DCM (50 mL×3). The combined organic phase was washed with brine (50 mL), dried over Na₂SO₄, filtered and concentrated under reduced pressure to give the title compound (1.90 g, 5.63 mmol, 94.4% yield) as a yellow solid. ¹H NMR (DMSO-d₆, 400 MHz) δ 7.64 (d, J 1.2 Hz, 1H), 7.60 (d, J=2.0 Hz, 1H), 5.33 (d, J=4.4 Hz, 1H), 5.26-5.17 (m, 1H), 3.51-3.48 (m, 4H), 2.39 (s, 3H), 2.23-2.08 (m, 4H), 1.92 (s, 3H), 1.41 (d, J=6.4 Hz, 3H).

### Preparation of 8-(1-bromoethyl)-2-(4,4-difluoro-1-piperidyl)-3,6-dimethyl-chromen-4-one

To a solution of 2-(4,4-difluoro-1-piperidyl)-8-(1-hydroxyethyl)-3,6-dimethyl-chromen-4-one (2.0 g, 5.93 mmol, 1 eq) in DCM (50 mL)/DMF (4.33 mg, 59.2 μmol, 4.56 μL, 0.01 eq) was added dropwise SOBr₂ (1.85 g, 8.89 mmol, 688 μL, 1.5 eq) at 0° C., the reaction mixture was warmed to 20° C. and stirred 20° C. for 20 min. The reaction mixture was poured into ice H₂O (100 mL) at 0° C. and extracted with DCM (100 mL×3). The combined organic layers were washed with sat.aq NaHCO₃ until pH=8, then washed with brine (100 mL), dried over Na₂SO₄, filtered and concentrated under reduced pressure to give the title compound (4.1 g, 10.2 mmol, 86.3% yield) as a yellow solid. ¹H NMR (DMSO-d₆, 400 MHz) δ 7.75 (s, 2H), 5.90-5.84 (m, 1H), 3.60-3.52 (m, 4H), 2.44-2.41 (m, 3H), 2.26-2.15 (m, 4H), 2.14-2.10 (m, 3H), 1.95-1.93 (m, 3H).

### Preparation of 8-[1-[(2-bromo-6-chloro-3-pyridyl)amino]ethyl]-2-(4,4-difluoro-1-piperidyl)-3,6-dimethyl-chromen-4-one

The title compound was prepared as a brown solid (600 mg, 1.14 mmol, 91.1% yield) according to the procedures described herein and the synthetic scheme, using 2-bromo-6-chloro-pyridin-3-amine (310 mg, 1.50 mmol, 1.2 eq) and 8-(1-bromoethyl)-2-(4,4-difluoro-1-piperidyl)-3,6-dimethyl-chromen-4-one (500 mg, 1.25 mmol, 1 eq). The product was used directly for next step without further purification.

### Preparation of 5-[6-chloro-3-[1-[2-(4,4-difluoro-1-piperidyl)-3,6-dimethyl-4-oxo-chromen-8-yl]ethylamino]-2-pyridyl]-2-hydroxy-benzaldehyde

The title compound was prepared as a yellow solid (0.5 g, 880 μmol, 77.2% yield) according to the procedures described herein and the synthetic scheme, using 8-[1-[(2-bromo-6-chloro-3-pyridyl)amino]ethyl]-2-(4,4-difluoro-1-piperidyl)-3,6-dimethyl-chromen-4-one (600 mg, 1.14 mmol, 1 eq) and 2-hydroxy-5-(4,4,5,5-tetramethyl-1,3,2-dioxaborolan-2-yl)benzaldehyde (367 mg, 1.48 mmol, 1.3 eq). ¹H NMR (DMSO-d₆, 400 MHz) δ 11.01 (s, 1H), 10.32 (s, 1H), 8.04 (d, J=2.0 Hz, 1H), 7.91 (dd, J=2.4, 8.8 Hz, 1H), 7.63 (d, J=1.2 Hz, 1H), 7.46 (d, J=2.0 Hz, 1H), 7.13 (d, J=8.8 Hz, 1H), 7.08 (d, J=8.8 Hz, 1H), 6.88 (d, J=8.8 Hz, 1H), 5.65 (d, J=7.2 Hz, 1H), 4.98-4.88 (m, 1H), 3.52-3.45 (m, 4H), 2.31 (s, 3H), 2.22-2.06 (m, 4H), 1.94 (s, 3H), 1.50 (d, J=6.4 Hz, 3H).

### Preparation of [4-[6-chloro-3-[1-[2-(4,4-difluoro-1-piperidyl)-3,6-dimethyl-4-oxo-chromen-8-yl]ethylamino]-2-pyridyl]-2-formyl-phenyl]trifluoromethanesulfonate

The title compound was prepared as a brown solid (1.7 g, 2.43 mmol, 68.9% yield) according to the procedures described herein and the synthetic scheme, using 5-[6-chloro-3-[1-[2-(4,4-difluoro-1-piperidyl)-3,6-dimethyl-4-oxo-chromen-8-yl]ethylamino]-2-pyridyl]-2-hydroxy-benzaldehyde (2.00 g, 3.52 mmol, 1 eq) and 1,1,1-trifluoro-N-phenyl-N-(trifluoromethylsulfonyl)methanesulfonamide (1.51 g, 4.23 mmol, 1.2 eq). ¹H NMR (DMSO-d₆, 400 MHz) δ 10.15 (s, 1H), 8.47 (d, J=2.4 Hz, 1H), 8.27 (dd, J=2.4, 8.4 Hz, 1H), 7.72 (d, J=8.4 Hz, 1H), 7.64 (d, J 1.2 Hz, 1H), 7.46 (d, J=2.0 Hz, 1H), 7.17 (d, J=8.8 Hz, 1H), 6.91 (d, J=8.8 Hz, 1H), 6.15 (d, J=6.8 Hz, 1H), 4.97-4.94 (m, 1H), 3.56-3.48 (m, 4H), 2.31 (s, 3H), 2.20-2.09 (m, 4H), 1.95 (s, 3H), 1.52 (d, J=6.4 Hz, 3H).

### Preparation of 5-[6-chloro-3-[1-[2-(4,4-difluoro-1-piperidyl)-3,6-dimethyl-4-oxo-chromen-8-yl]ethylamino]-2-pyridyl]-2-(4,4,5,5-tetramethyl-1,3,2-dioxaborolan-2-yl)benzaldehyde

The title compound was prepared as a yellow solid (0.5 g, 737 μmol, 43.0% yield) according to the procedures described herein and the synthetic scheme, using [4-[6-chloro-3-[1-[2-(4,4-difluoro-1-piperidyl)-3,6-dimethyl-4-oxo-chromen-8-yl]ethylamino]-2-pyridyl]-2-formyl-phenyl]trifluoromethanesulfonate (1.20 g, 1.71 mmol, 1 eq) and 4,4,5,5-tetramethyl-2-(4,4,5,5-tetramethyl-1,3,2-dioxaborolan-2-yl)-1,3,2-dioxaborolane (1.74 g, 6.86 mmol, 4 eq). ¹H NMR (DMSO-d₆, 400 MHz) δ 10.39 (s, 1H), 8.23 (d, J=1.2 Hz, 1H), 8.05 (dd, J=1.6, 7.6 Hz, 1H), 7.85 (d, J=7.6 Hz, 1H), 7.63 (d, J=1.2 Hz, 1H), 7.46 (d, J=2.0 Hz, 1H), 7.16 (d, J=8.4 Hz, 1H), 6.98 (d, J=8.8 Hz, 1H), 5.80 (d, J=7.2 Hz, 1H), 4.95-4.88 (m, 1H), 3.46-3.42 (m, 4H), 2.31 (s, 3H), 2.16-2.06 (m, 4H), 1.93 (s, 3H), 1.48 (d, J=6.4 Hz, 3H), 1.37 (s, 12H).

### Preparation of 8-[1-[[6-chloro-2-(1-hydroxy-2,3,1-benzoxazaborinin-6-yl)-3-pyridyl]amino]ethyl]-2-(4,4-difluoro-1-piperidyl)-3,6-dimethyl-chromen-4-one

The title compound was prepared as a white solid (350 mg, 590 μmol, 88.9% yield) according to the procedures described herein and the synthetic scheme, using 5-[6-chloro-3-[1-[2-(4,4-difluoro-1-piperidyl)-3,6-dimethyl-4-oxo-chromen-8-yl]ethylamino]-2-pyridyl]-2-(4,4,5,5-tetramethyl-1,3,2-dioxaborolan-2-yl)benzaldehyde (450 mg, 663 μmol, 1 eq) and hydroxylamine;hydrochloride (69.1 mg, 995 μmol, 1.5 eq). ¹H NMR (DMSO-d₆, 400 MHz) δ 9.48 (s, 1H), 8.73 (s, 1H), 8.18 (d, J=7.6 Hz, 1H), 8.09-8.05 (m, 2H), 7.64 (d, J=1.2 Hz, 1H), 7.46 (d, J=2.0 Hz, 1H), 7.17 (d, J=8.8 Hz, 1H), 6.95 (d, J=8.8 Hz, 1H), 5.87 (d, J=6.8 Hz, 1H), 4.96-4.89 (m, 1H), 3.47-3.42 (m, 4H), 2.32 (s, 3H), 2.19-2.06 (m, 4H), 1.93 (s, 3H), 1.51 (d, J=6.8 Hz, 3H).

### Preparation of 8-[(1S)-1-[[6-chloro-2-(1-hydroxy-2,3,1-benzoxazaborinin-6-yl)-3-pyridyl]amino]ethyl]-2-(4,4-difluoro-1-piperidyl)-3,6-dimethyl-chromen-4-one and 8-[(1R)-1-[[6-chloro-2-(1-hydroxy-2,3,1-benzoxazaborinin-6-yl)-3-pyridyl]amino]ethyl]-2-(4,4-difluoro-1-piperidyl)-3,6-dimethyl-chromen-4-one

8-[1-[[6-chloro-2-(1-hydroxy-2,3,1-benzoxazaborinin-6-yl)-3-pyridyl]amino]ethyl]-2-(4,4-difluoro-1-piperidyl)-3,6-dimethyl-chromen-4-one (0.3 g) was separated by prep-SFC (column: DAICEL CHIRALPAK IG (250 mm×30 mm, 10 um); mobile phase: [CO₂—IPA]; B %:50%, isocratic elution mode) to give the title compound, Isomer 1 (93.3 mg, 157 μmol, 93.3% yield) as a white solid and the title compound, Isomer 2 (76.7 mg, 129 μmol, 76.7% yield) as a white solid. Isomer 1: ¹H NMR (DMSO-d₆, 400 MHz) δ 9.43 (s, 1H), 8.73 (s, 1H), 8.18 (d, J=7.6 Hz, 1H), 8.09 (s, 1H), 8.07-8.05 (m, 1H), 7.64 (s, 1H), 7.47 (d, J=1.6 Hz, 1H), 7.17 (d, J=8.8 Hz, 1H), 6.96 (d, J=8.8 Hz, 1H), 5.87 (d, J=6.8 Hz, 1H), 4.97-4.91 (m, 1H), 3.46 (t, J=5.2 Hz, 4H), 2.33 (s, 3H), 2.21-2.04 (m, 4H), 1.94 (s, 3H), 1.51 (d, J=6.4 Hz, 3H). MS (ESI): mass calcd. For C₃₀H₂₈BClF₂N₄O₄ 592.19, m/z found 593.1 [M+H]⁺. HPLC: 99.84% (220 nm), 99.94% (254 nm). Chiral purity: 100% ee. Isomer 2: ¹H NMR (DMSO-d₆, 400 MHz) δ 9.40 (s, 1H), 8.71 (s, 1H), 8.17 (d, J=7.6 Hz, 1H), 8.06 (d, J=8.0 Hz, 1H), 8.04 (s, 1H), 7.63 (s, 1H), 7.46 (d, J=2.0 Hz, 1H), 7.16 (d, J=8.8 Hz, 1H), 6.95 (d, J=8.8 Hz, 1H), 5.86 (d, J=6.8 Hz, 1H), 4.96-4.89 (m, 1H), 3.46-3.43 (m, 4H), 2.32 (s, 3H), 2.16-2.07 (m, 4H), 1.93 (s, 3H), 1.50 (d, J=6.4 Hz, 3H). MS (ESI): mass calcd. For C₃₀H₂₈BClF₂N₄O₄ 592.19, m/z found 593.0 [M+H]⁺. HPLC: 99.79% (220 nm), 99.92% (254 nm). Chiral purity: 97.18% ee.

### Example 75

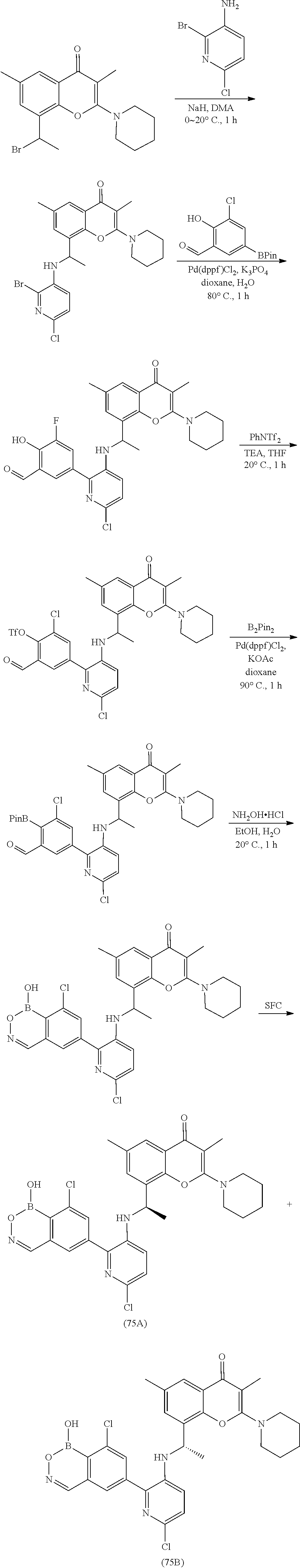

### Preparation of 8-[1-[(2-bromo-6-chloro-3-pyridyl)amino]ethyl]-3,6-dimethyl-2-(1-piperidyl)chromen-4-one

To a solution of 2-bromo-6-chloro-pyridin-3-amine (683 mg, 3.29 mmol, 1.2 eq) in DMA (10 nL) was added NaH (143 mg, 3.57 mmol, 1.3 eq) in one portion at 0° C., the resulting reaction mixture ws stirred at 0° C. for 1 h. A solution of 8-(1-bromoethyl)-3,6-dimethyl-2-(1-piperidyl)chromen-4-one (1.0 g, 2.75 mmol, 1 eq) in DMA (5 mL) was added dropwise to the reaction mixture at 0° C. Then the reaction mixture was stirred at 20° C. for 1 h. The reaction mixture was poured into ice-water (w/w=1/1) (50 mL) and the resulting suspension was filtered. The filter cake was washed with H₂O (10 mL) and dried in vacuum. The crude product was triturated with H₂O at 20° C. for 10 min to give the title compound (1.3 g, 2.65 mmol, 96.48% yield) as a yellow solid. ¹H NMR (DMSO-d₆, 400 MHz) δ 7.89 (s, 1H), 7.27 (s, 1H), 6.96 (d, J=8.4 Hz, 1H), 6.47 (d, J=8.4 Hz, 1H), 4.92-4.89 (m, 1H), 4.82-4.81 (m, 1H), 3.39-3.38 (m, 4H), 2.37 (s, 3H), 2.09 (s, 3H), 2.06 (s, 3H), 1.68-1.61 (m, 6H).

### Preparation of 3-chloro-5-[6-chloro-3-[1-[3,6-dimethyl-4-oxo-2-(1-piperidyl)chromen-8-yl]ethylamino]-2-pyridyl]-2-hydroxy-benzaldehyde

A mixture of 8-[1-[(2-bromo-6-chloro-3-pyridyl)amino]ethyl]-3,6-dimethyl-2-(1-piperidyl) chromen-4-one (1.2 g, 2.44 mmol, 1 eq), 3-chloro-2-hydroxy-5-(4,4,5,5-tetramethyl-1,3,2-dioxaborolan-2-yl) benzaldehyde (829 mg, 2.93 mmol, 1.2 eq), Pd(dppf)Cl₂ (179 mg, 245 μmol, 0.1 eq) and K₃PO₄ (1.04 g, 4.89 mmol, 2 eq) in dioxane (20 mL) and H₂O (2 mL) was de-gassed and then heated to 80° C. for 2 h under N₂. The reaction mixture was cooled to 25° C., filtered and concentrated in vacuo.

The residue was purified by flash silica gel chromatography (ISCO®; 20 g SepaFlash® Silica Flash Column, Eluent of 0~40% Ethyl acetate/Petroleum ether gradient @100 mL/min) to give the title compound (1.3 g, 2.29 mmol, 93.87% yield) as a yellow solid. ¹H NMR (DMSO-d₆, 400 MHz) δ 11.63 (s, 1H), 9.97 (s, 1H), 8.05 (s, 1H), 7.92-7.90 (m, 2H), 7.26 (s, 1H), 7.02 (d, J=8.4 Hz, 1H), 6.69 (d, J=8.8 Hz, 1H), 4.92-4.86 (m, 1H), 4.39-4.38 (m, 1H), 3.33-3.26 (m, 4H), 2.38 (s, 3H), 1.68 (s, 3H), 1.59 (d, J=6.8 Hz, 3H), 1.27 (s, 6H).

### Preparation of [2-chloro-4-[6-chloro-3-[[(R)-1-[3,6-dimethyl-4-oxo-2-(1-piperidyl) chromen-8-yl]ethyl]amino]-2-pyridyl]-6-formyl-phenyl]trifluoromethanesulfonate

To a solution of 3-chloro-5-[6-chloro-3-[1-[3,6-dimethyl-4-oxo-2-(1-piperidyl)chromen-8-yl]ethylamino]-2-pyridyl]-2-hydroxy-benzaldehyde (1.2 g, 2.12 mmol, 1 eq) and 1,1,1-trifluoro-N-phenyl-N-(trifluoromethylsulfonyl)methanesulfonamide (1.51 g, 4.24 mmol, 2 eq) in THF (30 mL) was added TEA (429 mg, 4.24 mmol, 2 eq) and DMAP (25.9 mg, 212 μmol, 0.1 eq) in turns at 20° C., the reaction mixture was stirred at 20° C. for 1 h. The reaction mixture was concentrated in vacuo and the residue was purified by flash silica gel chromatography (ISCO®; 20 g SepaFlash® Silica Flash Column, Eluent of 0~40% Ethyl acetate/Petroleum ether gradient @80 mL/min) to give the title compound (1.0 g, 1.43 mmol, 67.58% yield) as a yellow solid. ¹H NMR (DMSO-d₆, 400 MHz) δ 10.29 (s, 1H), 8.37 (d, J=2.0 Hz, 1H), 8.22 (d, J=2.0 Hz, 1H), 7.91 (d, J=4.2 Hz, 1H), 7.29 (d, J=2.0 Hz, 1H), 7.06 (d, J=8.4 Hz, 1H), 6.70 (d, J=8.8 Hz, 1H), 4.97-4.91 (m, 1H), 4.42-4.40 (m, 1H), 3.36-3.35 (m, 4H), 2.39 (s, 3H), 2.07 (s, 3H), 1.71 (s, 6H), 1.62 (d, J=6.8 Hz, 3H).

### Preparation of 3-chloro-5-(6-chloro-3-((1-(3,6-dimethyl-4-oxo-2-(piperidin-1-yl)-4H-chromen-8-yl)ethyl)amino)pyridin-2-yl)-2-(4,4,5,5-tetramethyl-1,3,2-dioxaborolan-2-yl)benzaldehyde

To a solution of [2-chloro-4-[6-chloro-3-[[(1R)-1-[3,6-dimethyl-4-oxo-2-(1-piperidyl) chromen-8-yl]ethyl]amino]-2-pyridyl]-6-formyl-phenyl]trifluoromethanesulfonate (0.3 g, 429 μmol, 1 eq) and B₂Pin₂ (436 mg, 1.72 mmol, 4 eq) in dioxane (10 mL) was added KOAc (126 mg, 1.29 mmol, 3 eq) and Pd(PPh₃)₂Cl₂ (30.1 mg, 42.9 μmol, 0.1 eq) in turns at 20° C., the reaction mixture was heated to 90° C. and stirred at 90° C. for 1 h. The mixture was cooled to 20° C., filtered through the Celite pad and the filtrate was concentrated under reduced pressure to give the title compound (400 mg, crude) as a yellow solid, which was used directly for next step.

### Preparation of 8-[1-[[6-chloro-2-(8-chloro-1-hydroxy-2,3,1-benzoxazaborinin-6-yl)-3-pyridyl]amino]ethyl]-3,6-dimethyl-2-(1-piperidyl)chromen-4-one

To a solution of 3-chloro-5-[6-chloro-3-[[(1R)-1-[3,6-dimethyl-4-oxo-2-(1-piperidyl)chromen-8-yl]ethyl]amino]-2-pyridyl]-2-(4,4,5,5-tetramethyl-1,3,2-dioxaborolan-2-yl)benzaldehyde (400 mg, 591 μmol, 1 eq) in EtOH (10 mL)/H₂O (1 mL) were added KOAc (116 mg, 1.18 mmol, 2 eq) and hydroxylamine;hydrochloride (83 mg, 1.18 mmol, 2 eq) in turns at 20° C., the reaction mixture was stirred at 20° C. for 1 h. The reaction mixture was concentrated in vacuo and the residue was directly purified by prep-HPLC (column: Waters Xbridge BEH C18 100×30 mm×10 um; mobile phase: [H₂O (10 mM NH₄HCO₃)-AC□]; gradient: 25%-55% B over 8.0 min) to give the title compound (30 mg, crude) as a yellow solid.

### Preparation of 8-[(1S)-1-[[6-chloro-2-(8-chloro-1-hydroxy-2,3,1-benzoxazaborinin-6-yl)-3-pyridyl]amino]ethyl]-3,6-dimethyl-2-(1-piperidyl)chromen-4-one & 8-[(1R)-1-[[6-chloro-2-(8-chloro-1-hydroxy-2,3,1-benzoxazaborinin-6-yl)-3-pyridyl]amino]ethyl]-3,6-dimethyl-2-(1-piperidyl)chromen-4-one

A mixture of 8-[1-[[6-chloro-2-(8-chloro-1-hydroxy-2,3,1-benzoxazaborinin-6-yl)-3-pyridyl]amino]ethyl]-3,6-dimethyl-2-(1-piperidyl)chromen-4-one (26 mg, 43.9 μmol, 1 eq) in EtOH (1 mL) was separated by SFC (column: DAICEL CHIRALPAK IK (250 mm×25 mm×10 um); mobile phase: [CO₂-EtOH (0.1% NH₃H₂O)]; B %:60%, isocratic elution mode) to give the title compound, Isomer 1 (9.1 mg, 15.06 μmol, 34.25% yield) as a white solid and the title compound, Isomer 2 (9.4 mg, 15.60 mol, 35.47% yield) as a white solid. Isomer 1: ¹H NMR (DMSO-d₆, 400 MHz) δ 9.35 (s, 1H), 8.71 (s, 1H), 8.01 (d, J=8.8 Hz, 2H), 7.61 (s, 1H), 7.45 (d, J=2.0 Hz, 1H), 7.21 (d, J=8.4 Hz, 1H), 7.01 (d, J=8.8 Hz, 1H), 6.06 (d, J=7.2 Hz, 1H), 4.95-4.88 (m, 1H), 3.30-3.27 (m, 4H), 2.32 (s, 3H), 1.89 (s, 3H), 1.60-1.59 (m, 6H), 1.51 (d, J=6.8 Hz, 3H). MS (ESI): mass calcd. For C₃₃H₂₉BCl₂N₄O₄ 590.17, m/z found 591.2 [M+H]⁺. HPLC: 97.87% (220 nm), 98.31% (254 nm). Chiral purity: 100 ee %. Isomer 2: ¹H NMR (DMSO-d₆, 400 MHz) δ 9.35 (brs, 1H), 8.70 (s, 1H), 8.01 (d, J=8.8 Hz, 2H), 7.61 (d, J=1.2 Hz, 1H), 7.45 (d, J=2.0 Hz, 1H), 7.21 (d, J=8.4 Hz, 1H), 7.01 (d, J=8.8 Hz, 1H), 6.05 (d, J=7.2 Hz, 1H), 4.95-4.89 (m, 1H), 3.28-3.27 (m, 4H), 2.32 (s, 3H), 1.89 (s, 3H), 1.60-1.59 (m, 6H), 1.51 (d, J=6.8 Example 76 Hz, 3H). MS (ESI): mass calcd. For C₃₃H₂₉BCl₂N₄O₄ 590.17, m/z found 591.2 [M+H]⁺. HPLC: 98.11% (220 nm), 98.72% (254 nm). Chiral purity: 97.12 ee %.

### Example 76

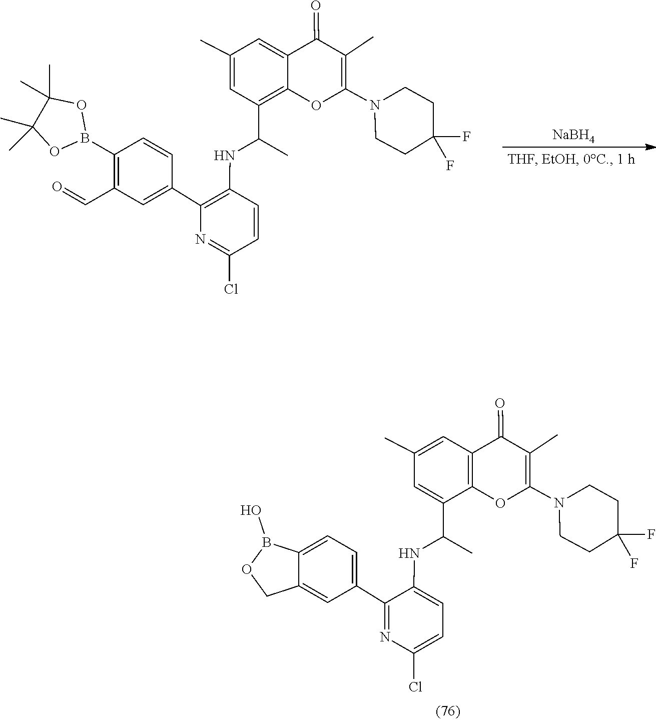 [SMILES: Cc1cc(C(C)Nc2ccc(Cl)nc2-c2ccc(B3OC(C)(C)C(C)(C)O3)c(C=O)c2)c2oc(N3CCC(F)(F)CC3)c(C)c(=O)c2c1 ; "NaBH4" ; Cc1cc(C(C)Nc2ccc(Cl)nc2-c2ccc3c(c2)COB3O)c2oc(N3CCC(F)(F)CC3)c(C)c(=O)c2c1]

### Preparation of 8-[1-[[6-chloro-2-(1-hydroxy-3H-2,1-benzoxaborol-5-yl)-3-pyridyl]amino]ethyl]-2-(4,4-difluoro-1-piperidyl)-3,6-dimethyl-chromen-4-one

To a solution of 5-[6-chloro-3-[1-[2-(4,4-difluoro-1-piperidyl)-3,6-dimethyl-4-oxo-chromen-8-yl]ethylamino]-2-pyridyl]-2-(4,4,5,5-tetramethyl-1,3,2-dioxaborolan-2-yl)benzaldehyde (100 mg, 147 μmol, 1 eq) in THF (2 mL)/EtOH (0.5 mL) was added NaBH₄ (27.9 mg, 737 μmol, 5 eq) in one portion at 0° C., the reaction mixture was stirred at 0° C. for 1 h. The reaction mixture was poured into ice water (20 mL) at 0° C., adjusted pH=5 with 1N HCl and then extracted with DCM (20 mL×3). The combined organic phase was washed with brine (20 mL), dried over Na₂SO₄, filtered and concentrated under reduced pressure to give a residue. The residue was purified by prep-HPLC (column: Waters Xbridge Prep OBD C18 150×40 mm×10 um; mobile phase: [H₂O (10 mM NH₄HCO₃)-AC□]; gradient: 35%-65% B over 8.0 min) to give the title compound (5.2 mg, 8.62 μmol, 5.85% yield) as a white solid. ¹H NMR (DMSO-d₆, 400 MHz) δ 9.27 (br s, 1H), 7.85 (d, J=8.0 Hz, 1H), 7.68-7.66 (m, 2H), 7.64 (s, 1H), 7.44 (d, J=1.6 Hz, 1H), 7.12 (d, J=8.4 Hz, 1H), 6.92 (d, J=8.8 Hz, 1H), 5.54 (d, J=6.8 Hz, 1H), 5.04-4.98 (m, 2H), 4.96-4.91 (m, 1H), 3.44-3.43 (m, 4H), 2.32 (s, 3H), 2.15-2.07 (m, 4H), 1.93 (s, 3H), 1.50 (d, J=6.8 Hz, 3H). MS (ESI): mass calcd. For C₃₀H₂₉BClF₂N₃O₄ 579.19, m/z found 580.1 [M+H]⁺. HPLC: 96.78% (220 nm), 97.25% (254 nm).

### Example 77

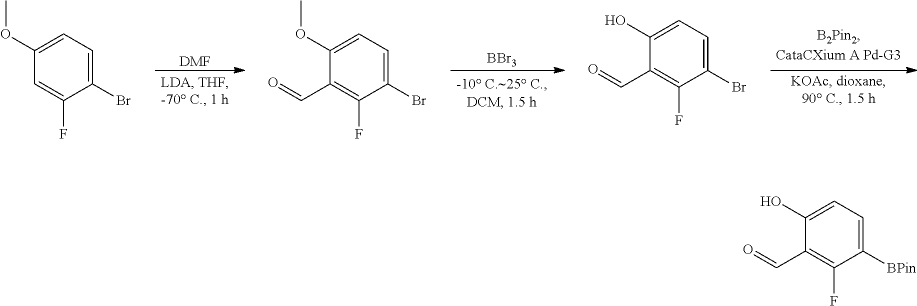

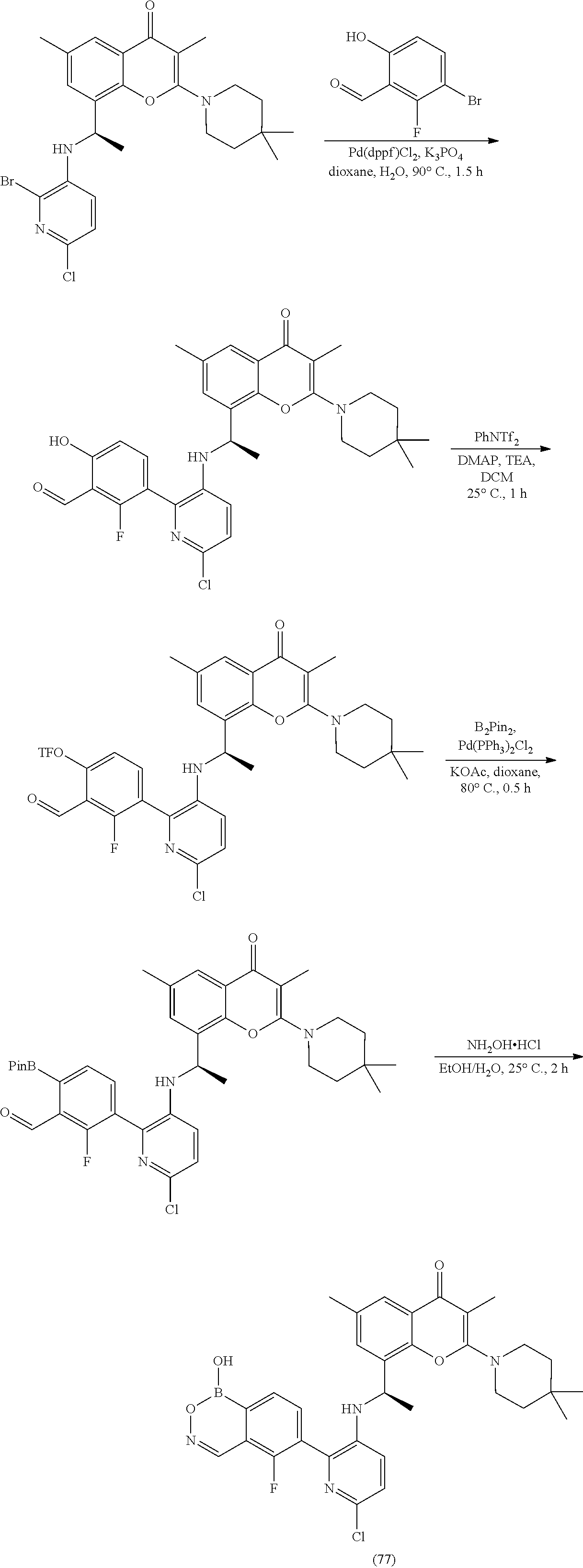

### Preparation of 3-bromo-2-fluoro-6-methoxy-benzaldehyde

To a solution of 1-bromo-2-fluoro-4-methoxy-benzene (5.00 g, 24.39 mmol, 1 eq) in THF (50 mL) was added LDA (2 M, 18.29 mL, 1.5 eq) dropwise at −70° C., the reaction mixture was stirred at −70° C. for 0.5 h. After that, DMF (3.57 g, 48.77 mmol, 3.75 mL, 2 eq) was added dropwise to the reaction mixture at −70° C., the resulting reaction mixture was stirred at −70° C. for additional 0.5 h. The reaction mixture was poured into aq.NH₄Cl (50 mL) at 0° C. under N₂ and the solid formed. Then the solid was collected by filtration, washed with H₂O (10 mL×2) and dried in vacuo to give the title compound (4.75 g, 20.38 mmol, 83.58% yield) as a pale-white solid. ¹H NMR (DMSO-d₆, 400 MHz) δ 10.24 (s, 1H), 7.95-7.91 (m, 1H), 7.06 (d, J=9.2 Hz, 1H), 3.92 (s, 3H).

### Preparation of 3-bromo-2-fluoro-6-hydroxy-benzaldehyde

To a solution of 3-bromo-2-fluoro-6-methoxy-benzaldehyde (5.60 g, 24.03 mmol, 1 eq) in DCM (60 mL) was added BBr₃ (2 M, 34.00 mL, 2.83 eq) dropwise at −10° C. under N₂, the resulting reaction mixture was allowed to warm to 25° C. and stirred at 25° C. for 1.5 h. The reaction mixture was poured into ice-water (50 mL) and extracted with DCM (50 mL×3). The combined organic layers were washed with aq.NaHCO₃ (50 mL) and brine (50 mL), dried over Na₂SO₄, filtered and concentrated under reduced pressure to give the title compound (4.75 g, 21.17 mmol, 88.08% yield) as a yellow solid. ¹H NMR (DMSO-d₆, 400 MHz) δ 11.34 (s, 1H), 10.22 (s, 1H), 7.77 (t, J=8.4 Hz, 1H), 6.82 (d, J=8.8 Hz, 1H).

### Preparation of 2-fluoro-6-hydroxy-3-(4,4,5,5-tetramethyl-1,3,2-dioxaborolan-2-yl)benzaldehyde

To a mixture of 3-bromo-2-fluoro-6-hydroxy-benzaldehyde (3.20 g, 14.61 mmol, 1 eq), B₂Pin₂ (5.57 g, 21.92 mmol, 1.5 eq) and KOAc (2.87 g, 29.22 mmol, 2 eq) in dioxane (40 mL) was added [2-(2-aminophenyl)phenyl]palladium(1+);bis(1-adamantyl)-butyl-phosphane;metha nesulfonate (1.06 g, 1.46 mmol, 0.1 eq) in one portion at 25° C. under N₂, the reaction mixture was degassed and purged with N₂ for 3 times, heated to 90° C. and stirred at 90° C. for 1.5 h under N₂ atmosphere. The reaction mixture was cooled to 20° C., filtered, and the filtered cake was washed with EtOAc (30 mL×2). The combined organic layers were concentrated under reduced pressure and the residue was purified by flash silica gel chromatography (Biotage®; 120 g SepaFlash® Silica Flash Column, Eluent of 0~25% Ethyl acetate/Petroleum ether gradient @100 mL/min) to give the title compound (2.25 g, 8.46 mmol, 57.88% yield) as a black solid. ¹H NMR (DMSO-d₆, 400 MHz) δ 11.55 (s, 1H), 10.25 (s, 1H), 7.72 (t, J=8.0 Hz, 1H), 6.81 (d, J=8.4 Hz, 1H), 1.28 (s, 12H).

### Preparation of 3-[6-chloro-3-[[(1R)-1-[2-(4,4-dimethyl-1-piperidyl)-3,6-dimethyl-4-oxo-chromen-8-yl]ethyl]amino]-2-pyridyl]-2-fluoro-6-hydroxy-benzaldehyde

The title compound was prepared as a yellow solid (1.11 g, crude) according to the procedures described herein and the synthetic scheme, using 8-[(1R)-1-[(2-bromo-6-chloro-3-pyridyl)amino]ethyl]-2-(4,4-dimethyl-1-piperidyl)-3,6-dimethyl-chromen-4-one (1 g, 1.93 mmol, 1 eq) and 2-fluoro-6-hydroxy-3-(4,4,5,5-tetramethyl-1,3,2-dioxaborolan-2-yl)benzaldehyde (769.18 mg, 2.89 mmol, 1.5 eq). The product was used directly for next step without further purification.

### Preparation of [4-[6-chloro-3-[[(1R)-1-[2-(4,4-dimethyl-1-piperidyl)-3,6-dimethyl-4-oxo-chromen-8-yl]ethyl]amino]-2-pyridyl]-3-fluoro-2-formyl-phenyl]trifluoromethanesulfonate

The title compound was prepared as a yellow solid (1.20 g, 1.69 mmol, 97.68% yield) according to the procedures described herein and the synthetic scheme, using 3-[6-chloro-3-[[(1R)-1-[2-(4,4-dimethyl-1-piperidyl)-3,6-dimethyl-4-oxo-chromen-8-yl]ethyl]amino]-2-pyridyl]-2-fluoro-6-hydroxy-benzaldehyde (1.00 g, 1.73 mmol, 1 eq) and PhNTf₂ (927 mg, 2.59 mmol, 1.5 eq). ¹H NMR (DMSO-d₆, 400 MHz) δ 10.34 (s, 1H), 8.03 (t, J=8.4 Hz, 1H), 7.63 (s, 1H), 7.57 (d, J=4.4 Hz, 1H), 7.38 (d, J=1.6 Hz, 1H), 7.23 (d, J=2.0 Hz, 1H), 6.91 (d, J=9.6 Hz, 1H), 5.98 (d, J=6.8 Hz, 1H), 5.03-4.96 (m, 1H), 3.41-3.36 (m, 4H), 2.31 (s, 3H), 1.92 (s, 3H), 1.50 (d, J=6.4 Hz, 3H), 1.46 (t, J=5.2 Hz, 4H), 0.99 (s, 6H).

### Preparation of 3-[6-chloro-3-[[(1R)-1-[2-(4,4-dimethyl-1-piperidyl)-3,6-dimethyl-4-oxo-chromen-8-yl]ethyl]amino]-2-pyridyl]-2-fluoro-6-(4,4,5,5-tetramethyl-1,3,2-dioxaborolan-2-yl)benzaldehyde

The title compound was prepared as a gray solid (660 mg, crude) according to the procedures described herein and the synthetic scheme, using [4-[6-chloro-3-[[(1R)-1-[2-(4,4-dimethyl-1-piperidyl)-3,6-dimethyl-4-oxo-chromen-8-yl]ethyl]amino]-2-pyridyl]-3-fluoro-2-formyl-phenyl]trifluoromethanesulfonate (850 mg, 1.20 mmol, 1 eq) and B₂Pin₂ (912 mg, 3.59 mmol, 3 eq). The product was used directly for next step without further purification.

### Preparation of 8-[(1R)-1-[[6-chloro-2-(5-fluoro-1-hydroxy-2,3,1-benzoxazaborinin-6-yl)-3-pyridyl]amino]ethyl]-2-(4,4-dimethyl-1-piperidyl)-3,6-dimethyl-chromen-4-one

The title compound was prepared as a white solid (23.2 mg, 37.58 μmol, 8.62% yield) according to the procedures described herein and the synthetic scheme, using 3-[6-chloro-3-[[(1R)-1-[2-(4,4-dimethyl-1-piperidyl)-3,6-dimethyl-4-oxo-chromen-8-yl]ethyl]amino]-2-pyridyl]-2-fluoro-6-(4,4,5,5-tetramethyl-1,3,2-dioxaborolan-2-yl)benzaldehyde (300 mg, 436.03 μmol, 1 eq) and hydroxylamine;hydrochloride (45 mg, 654.04 μmol, 1.5 eq). ¹H NMR (DMSO-d₆, 400 MHz) δ 9.58-9.49 (br s, 1H), 8.78 (s, 1H), 7.95 (d, J=7.6 Hz, 1H), 7.82 (t, J=6.8 Hz, 1H), 7.61 (s, 1H), 7.38 (s, 1H), 7.23 (d, J=8.8 Hz, 1H), 6.96 (d, J=8.8 Hz, 1H), 5.77 (d, J=6.8 Hz, 1H), 5.00-4.89 (m, 1H), 3.57-3.47 (m, 4H), 2.32 (s, 3H), 1.91 (s, 3H), 1.47 (d, J=6.8 Hz, 3H), 1.44 (t, J=6.8 Hz, 4H), 0.98 (s, 6H). MS (ESI): mass calcd. For C₃₂H₃₃BClF₃N₄O₄ 602.23, m/z found 603.3 [M+H]⁺. HPLC: 97.66% (220 nm), 97.90% (254 nm). Chiral purity: 98.46% ee.

### Example 78

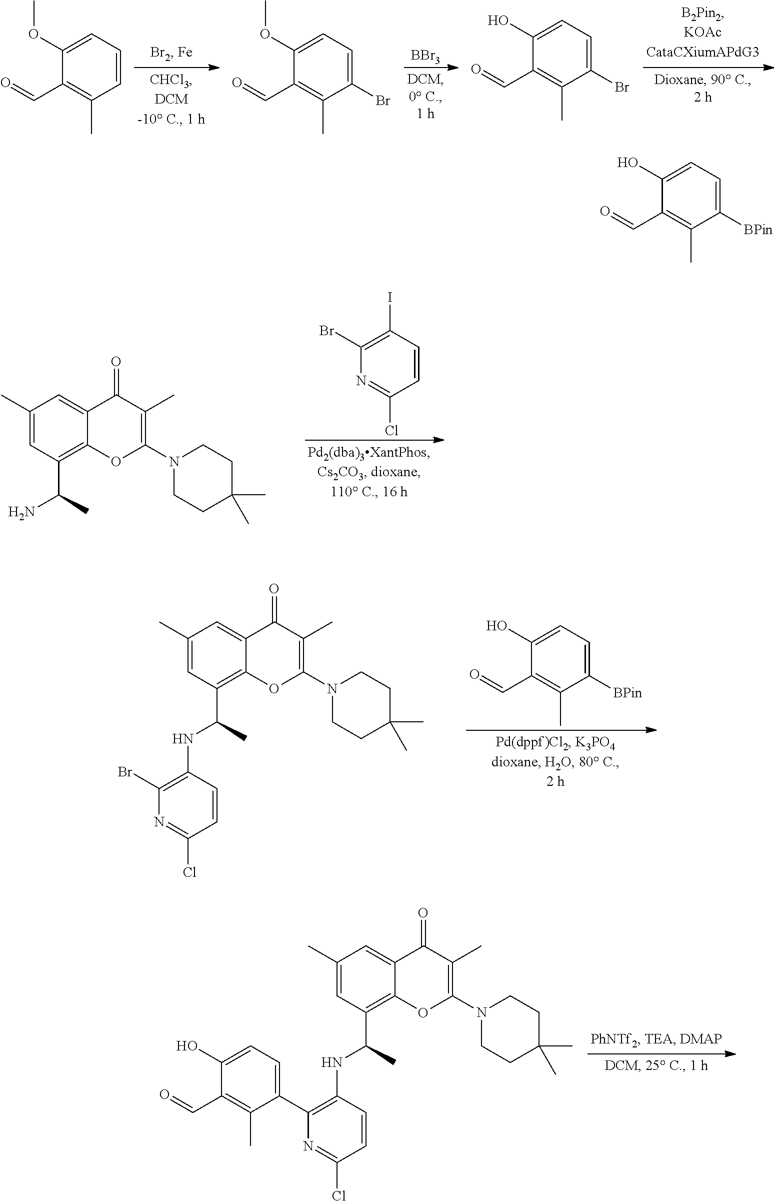

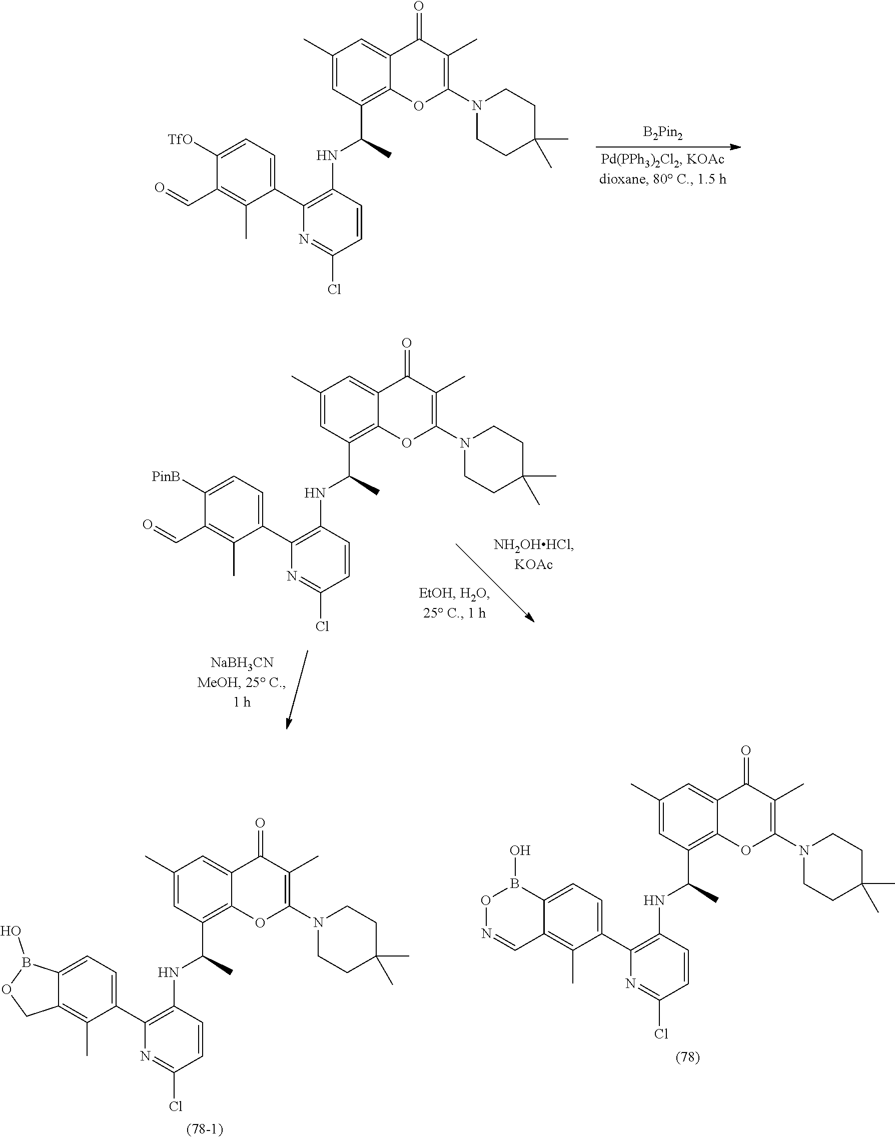

### Preparation of 3-bromo-6-methoxy-2-methyl-benzaldehyde

To a solution of 2-methoxy-6-methyl-benzaldehyde (3.90 g, 25.97 mmol, 1 eq) in CHCl₃ (40 mL) was added iron powder (73 mg, 1.30 mmol, 0.05 eq) in one portion at −10° C., the reaction mixture was stirred at −10° C. for 10 min. Then a solution of Br₂ (4.57 g, 28.57 mmol, 1.47 mL, 1.1 eq) in DCM (4 mL) was added dropwise into the reaction mixture at −10° C., the reaction mixture was stirred at −10° C. for 1 h. The reaction mixture was diluted with H₂O (40 mL) at −10° C. and then extracted with DCM (40 mL×3). The combined organic layers were washed with sodium thiosulfate solution (80 mL), brine (40 mL), dried over Na₂SO₄, filtered and concentrated under reduced pressure to give crude product. The crude product was triturated with PE (30 mL) at 25° C. for 10 min and then filtered. The filter cake was washed with PE (5 mL) and dried in vacuum to give the title compound (4.90 g, 21.39 mmol, 82.37% yield) as an orange solid. ¹H NMR (CDCl₃, 400 MHz) δ 12.57 (s, 1H), 7.68 (d, J=8.8 Hz, 1H), 6.76 (d, J=8.8 Hz, 1H), 3.90 (s, 3H), 2.65 (s, 3H).

### Preparation of 3-bromo-6-hydroxy-2-methyl-benzaldehyde

To a solution of 3-bromo-6-methoxy-2-methyl-benzaldehyde (6 g, 26.19 mmol, 1 eq) in DCM (60 mL) was added dropwise BBr₃ (2 M, 13.10 mL, 1 eq) at 0° C., the reaction mixture was stirred at 0° C. for 1 h. The reaction mixture was poured into ice-water (60 mL) and then extracted with DCM (60 mL×3). The combined organic layers were washed with aq. sat. NaHCO₃ (50 mL), brine (50 mL), dried over Na₂SO₄, filtered and concentrated under reduced pressure to give a residue. The crude product was triturated with PE (30 mL) at 25° C. for 10 min and then filtered. The filter cake was dried in vacuum to give the title compound (4.35 g, 20.18 mmol, 77.03% yield) as a light yellow solid. ¹H NMR (CDCl₃, 400 MHz) δ 12.08 (s, 1H), 10.38 (s, 1H), 7.65 (d, J=8.8 Hz, 1H), 6.76 (d, J=9.2 Hz, 1H), 2.70 (s, 3H).

### Preparation of 6-hydroxy-2-methyl-3-(4,4,5,5-tetramethyl-1,3,2-dioxaborolan-2-yl)benzaldehyde

A mixture of 3-bromo-6-hydroxy-2-methyl-benzaldehyde (3.70 g, 17.21 mmol, 1 eq), 4,4,5,5-tetramethyl-2-(4,4,5,5-tetramethyl-1,3,2-dioxaborolan-2-yl)-1,3,2-dioxaborolane (6.55 g, 25.81 mmol, 1.5 eq), [2-(2-aminophenyl)phenyl]palladium(1+);bis(1-adamantyl)-butyl-phosphan e;methanesulfonate (627 mg, 860.29 μmol, 0.05 eq) and KOAc (5.07 g, 51.62 mmol, 3 eq) in dioxane (40 mL) was degassed and purged with N₂ for 3 times. The reaction mixture was heated to 90° C. and stirred at 90° C. for 2 h under N₂ atmosphere. The reaction mixture was cooled to 25° C. and filtered through a pad of Celite. The filter cake was washed with EtOAc (20 mL×2) and the filtrate was concentrated under reduced pressure. The residue was purified by flash silica gel chromatography (Biotage®; 80 g SepaFlash® Silica Flash Column, Eluent of 0~5% Ethyl acetate/Petroleum ether gradient @80 mL/min) to give the title compound (4.00 g, 15.26 mmol, 88.70% yield) as a brown solid. ¹H NMR (CDCl₃, 400 MHz) δ 12.25 (s, 1H), 10.35 (s, 1H), 7.83 (d, J=8.4 Hz, 1H), 6.71 (d, J=8.8 Hz, 1H), 2.77 (s, 3H), 1.27 (s, 12H).

### Preparation of 8-[(1R)-1-[(2-bromo-6-chloro-3-pyridyl)amino]ethyl]-2-(4,4-dimethyl-1-piperidyl)-3,6-dimethyl-chromen-4-one

A mixture of 8-[(1R)-1-aminoethyl]-2-(4,4-dimethyl-1-piperidyl)-3,6-dimethyl-chromen-4-one (3.00 g, 9.13 mmol, 1 eq), 2-bromo-6-chloro-3-iodo-pyridine (4.36 g, 13.70 mmol, 1.5 eq), Cs₂CO₃ (5.95 g, 18.27 mmol, 2 eq), Xantphos (1.59 g, 2.74 mmol, 0.3 eq) and Pd₂(dba)₃ (836.41 mg, 913.39 μmol, 0.1 eq) in dioxane (50 mL) was degassed and purged with N₂ for 3 times, and then the reaction mixture was heated to 110° C. and stirred at 110° C. for 16 h under N₂ atmosphere. The reaction mixture was cooled to 25° C., filtered through a pad of Celite and the filter cake was washed with EtOAc (20 mL×2). The filtrate was concentrated under reduced pressure and the residue was purified by flash silica gel chromatography (Biotage®; 20 g SepaFlash® Silica Flash Column, Eluent of 0~40% Ethyl acetate/Petroleum ether gradient @60 mL/min) to give the title compound (3.90 g, 7.52 mmol, 82.29% yield) as yellow oil. ¹H NMR (DMSO-d₆, 400 MHz) δ 7.64 (s, 1H), 7.42 (d, J=2.0 Hz, 1H), 7.19 (d, J=8.8 Hz, 1H), 6.81 (d, J=8.8 Hz, 1H), 5.76-5.74 (m, 1H), 5.02 (q, J=7.2 Hz, 1H), 3.40-3.37 (m, 4H), 2.31 (s, 3H), 1.91 (s, 3H), 1.63 (d, J=6.8 Hz, 3H), 1.45-1.43 (m, 4H), 0.98 (s, 6H).

### Preparation of 3-[6-chloro-3-[[(1R)-1-[2-(4,4-dimethyl-1-piperidyl)-3,6-dimethyl-4-oxo-chromen-8-yl]ethyl]amino]-2-pyridyl]-6-hydroxy-2-methyl-benzaldehyde

A mixture of 8-[(1R)-1-[(2-bromo-6-chloro-3-pyridyl)amino]ethyl]-2-(4,4-dimethyl-1-piperidyl)-3,6-dimethyl-chromen-4-one (1.00 g, 1.93 mmol, 1 eq), 6-hydroxy-2-methyl-3-(4,4,5,5-tetramethyl-1,3,2-dioxaborolan-2-yl)benzaldehyde (657 mg, 2.51 mmol, 1.3 eq), K₃PO₄ (1.23 g, 5.78 mmol, 3 eq) and Pd(dppf)Cl₂ (142 mg, 192.73 μmol, 0.1 eq) in dioxane (20 mL) and H₂O (4 mL) was degassed and purged with N₂ for 3 times. The reaction mixture was heated to 80° C. and stirred at 80° C. for 2 h under N₂ atmosphere. 2 reactions were combined for work up. The reaction mixture was cooled to 25° C., diluted with H₂O (10 mL) and extracted with EtOAc (10 mL×2). The combined organic layers were dried over Na₂SO₄, filtered and concentrated under reduced pressure to give a residue. The residue was purified by flash silica gel chromatography (Biotage®; 20 g SepaFlash® Silica Flash Column, Eluent of 0~45% Ethyl acetate/Petroleum ether gradient @60 mL/min) to give the title compound (1.10 g, 1.92 mmol) as a yellow solid. ¹H NMR (DMSO-d₆, 400 MHz) δ 11.58 (s, 1H), 10.48 (d, J=12.0 Hz, 1H), 7.61 (s, 1H), 7.38-7.25 (m, 2H), 7.15 (d, J=8.8 Hz, 1H), 6.94-6.93 (m, 2H), 5.15-5.05 (m, 1H), 4.95 (q, J=7.2 Hz, 1H), 3.38-3.35 (m, 4H), 2.29 (s, 3H), 2.27 (s, 3H), 1.91 (s, 3H), 1.47-1.42 (m, 7H), 0.98 (s, 6H).

### Preparation of [4-[6-chloro-3-[[(1R)-1-[2-(4,4-dimethyl-1-piperidyl)-3,6-dimethyl-4-oxo-chromen-8-yl]ethyl]amino]-2-pyridyl]-2-formyl-3-methyl-phenyl]trifluoromethanesulfonate

To a solution of 3-[6-chloro-3-[[(1R)-1-[2-(4,4-dimethyl-1-piperidyl)-3,6-dimethyl-4-oxo-chromen-8-yl]ethyl]amino]-2-pyridyl]-6-hydroxy-2-methyl-benzaldehyde (1.00 g, 1.74 mmol, 1 eq), TEA (352 mg, 3.48 mmol, 484.89 μL, 2 eq) and DMAP (22 mg, 174.18 μmol, 0.1 eq) in DCM (10 mL) was added 1,1,1-trifluoro-N-phenyl-N-(trifluoromethylsulfonyl)methanesulfon amide (1.24 g, 3.48 mmol, 2 eq) in portions at 25° C., the reaction mixture was stirred at 25° C. for 1 h. The reaction was quenched with H₂O (10 mL) and extracted with DCM (10 mL×2). The combined organic layers were dried over Na₂SO₄, filtered and concentrated under reduced pressure to give a residue. The residue was purified by flash silica gel chromatography (Biotage®; 20 g SepaFlash® Silica Flash Column, Eluent of 0~45% Ethyl acetate/Petroleum ether gradient @60 mL/min) to give the title compound (1.10 g, 1.56 mmol, 89.43% yield) as a yellow solid. ¹H NMR (DMSO-d₆, 400 MHz) δ 10.50-10.45 (m, 1H), 7.69-7.68 (m, 1H), 7.62 (s, 1H), 7.52-7.50 (m, 1H), 7.34-7.27 (m, 1H), 7.21 (d, J=8.8 Hz, 1H), 6.96-6.94 (m, 1H), 5.53-5.43 (m, 1H), 4.97 (q, J=6.8 Hz, 1H), 3.37-3.36 (m, 4H), 2.41-2.40 (m, 3H), 2.30 (s, 3H), 1.91 (s, 3H), 1.46-1.44 (m, 7H), 0.99 (s, 6H).

### Preparation of 3-[6-chloro-3-[[(1R)-1-[2-(4,4-dimethyl-1-piperidyl)-3,6-dimethyl-4-oxo-chromen-8-yl]ethyl]amino]-2-pyridyl]-2-methyl-6-(4,4,5,5-tetramethyl-1,3,2-dioxaborolan-2-yl)benzaldehyde

A mixture of [4-[6-chloro-3-[[(1R)-1-[2-(4,4-dimethyl-1-piperidyl)-3,6-dimethyl-4-oxo-chromen-8-yl]ethyl]amino]-2-pyridyl]-2-formyl-3-methyl-phenyl]trifluoromethanesulfonate (700 mg, 991.26 μmol, 1 eq), B₂Pin₂ (756 mg, 2.97 mmol, 3 eq) and KOAc (195 mg, 1.98 mmol, 2 eq) and Pd(PPh₃)₂Cl₂ (70 mg, 99.13 μmol, 0.1 eq) in dioxane (10 mL) was degassed and purged with N₂ for 3 times. Then the reaction mixture was heated to 80° C. and stirred at 80° C. for 1.5 h under N₂ atmosphere. The reaction mixture was cooled to 25° C. and filtered through a pad of Celite. The filter cake was washed with EtOAc (10 mL) and the filtrate was concentrated under reduced pressure to give a residue. The residue was dissolved in MTBE (10 mL), stirred at 25° C. for 30 min, and filtered. The filtrate was concentrated under reduced pressure to give the title compound (1.5 g, crude) as brown oil, which was used directly for next step.

### Preparation of (R)-8-(1-((6-chloro-2-(1-hydroxy-5-methyl-JH-benzo[d][1,2,6]oxazab orinin-6-yl)pyridin-3-yl)amino)ethyl)-2-(4,4-dimethylpiperidin-1-yl)-3,6-dimethyl-4H-chromen-4-one

To a solution of 3-[6-chloro-3-[[(1R)-1-[2-(4,4-dimethyl-1-piperidyl)-3,6-dimethyl-4-oxo-chromen-8-yl]ethyl]amino]-2-pyridyl]-2-methyl-6-(4,4,5,5-tetramethyl-1,3,2-dioxaborolan-2-yl)benzaldehyde (1.0 g, 1.46 mmol, 1 eq) in EtOH (5 mL)/H₂O (1 mL) were added KOAc (287 mg, 2.92 mmol, 2 eq) and hydroxylamine; hydrochloride (153 mg, 2.19 mmol. 1.5 eq) in turns at 25° C., the reaction mixture was stirred at 25° C. for 1 h. The reaction mixture was concentrated under reduced pressure to give a residue. The residue was purified by prep-HPLC (column: Waters Xbridge BEH C18 100×30 mm×10 um; mobile phase: [H₂O (10 mM NH₄HCO₃)-MeOH]; gradient: 50%-90% B over 8.0 min) to give the crude product (95.97% purity). The crude product was further purified by prep-HPLC (column: Waters Xbridge BEH C18 100×25 mm×5 um; mobile phase: [H₂O (10 mM NH₄HCO₃)-AC□]; gradient: 20%-75% B over 8.0 min) to give the title compound (40.3 mg, 66.40 μmol, 4.54% yield) as a white solid. ¹H NMR (DMSO-d₆, 400 MHz) δ 9.40 (br s, 1H), 8.87-8.80 (m, 1H), 7.99-7.98 (m, 1H), 7.62-7.52 (m, 2H), 7.35-7.26 (m, 1H), 7.22-7.20 (m, 1H), 7.00-6.98 (m, 1H), 5.28-5.12 (m, 1H), 4.93 (q, J=6.8 Hz, 1H), 3.30-3.25 (m, 4H), 2.30-2.21 (m, 6H), 1.90 (s, 3H), 1.44-1.40 (m, 7H), 0.97 (s, 6H). MS (ESI): mass calcd. For C₃₃H₃₆BClN₄O₄ 598.25, m/z found 599.4 [M+H]⁺. HPLC: 98.68% (220 nm), 98.89% (254 nm). Chiral purity: 99.46% ee.

### Preparation of (R)-8-(1-((6-chloro-2-(1-hydroxy-4-methyl-1,3-dihydrobenzo[c][1,2]oxaborol-5-yl)pyridin-3-yl)amino)ethyl)-2-(4,4-dimethylpiperidin-1-yl)-3,6-dimethyl-4H-chromen-4-one

To a solution of 3-[6-chloro-3-[[(1R)-1-[2-(4,4-dimethyl-1-piperidyl)-3,6-dimethyl-4-oxo-chromen-8-yl]ethyl]amino]-2-pyridyl]-2-methyl-6-(4,4,5,5-tetramethyl-1,3,2-dioxaborolan-2-yl)benzaldehyde (500 mg, 730.92 μmol, 1 eq) in MeOH (5 mL) was added NaBH₃CN (69 mg, 1.10 mmol, 1.5 eq) at 0° C., the reaction mixture was warmed to 25° C. and stirred at 25° C. for 1 h. To the reaction mixture was quenched with aq.HCl (2N, 1 mL) at 0° C. under N₂ and extracted with EtOAc (3 mL×2).

The combined organic layers were washed with brine (3 mL), dried over Na₂SO₄, filtered and concentrated under reduced pressure. The residue was purified by prep-TLC (SiO₂, Petroleum ether:Ethyl acetate=1:1) to give the crude product. The crude product was purified by prep-HPLC (column: Waters Xbridge BEH C18 100×30 mm×10 um; mobile phase: [H₂O (10 mM NH₄HCO₃)-AC Q]; gradient: 55%-95% B over 8.0 min) to give the title compound (22 mg, 36.54 μmol, 5.00% yield) as a white solid. ¹H NMR (DMSO-d₆, 400 MHz) δ 9.23 (s, 1H), 7.65 (d, J=7.2 Hz, 1H), 7.61 (s, 1H), 7.32 (s, 1H), 7.22-7.18 (m, 2H), 7.04 (d, J=8.8 Hz, 1H), 4.97-4.90 (m, 4H), 3.31-3.24 (m, 4H), 2.29 (s, 3H), 1.90 (s, 3H), 1.87 (s, 3H), 1.46 (d, J=6.4 Hz, 3H), 3.42-3.39 (m, 4H), 0.97 (s, 6H). MS (ESI): mass calcd. For C₃₃H₃₇BClN₃O₄ 585.26, m/z found 586.3 [M+H]⁺. HPLC: 97.32% (220 nm), 97.20% (254 nm). Chiral purity: 98.22% ee.

### Example 79

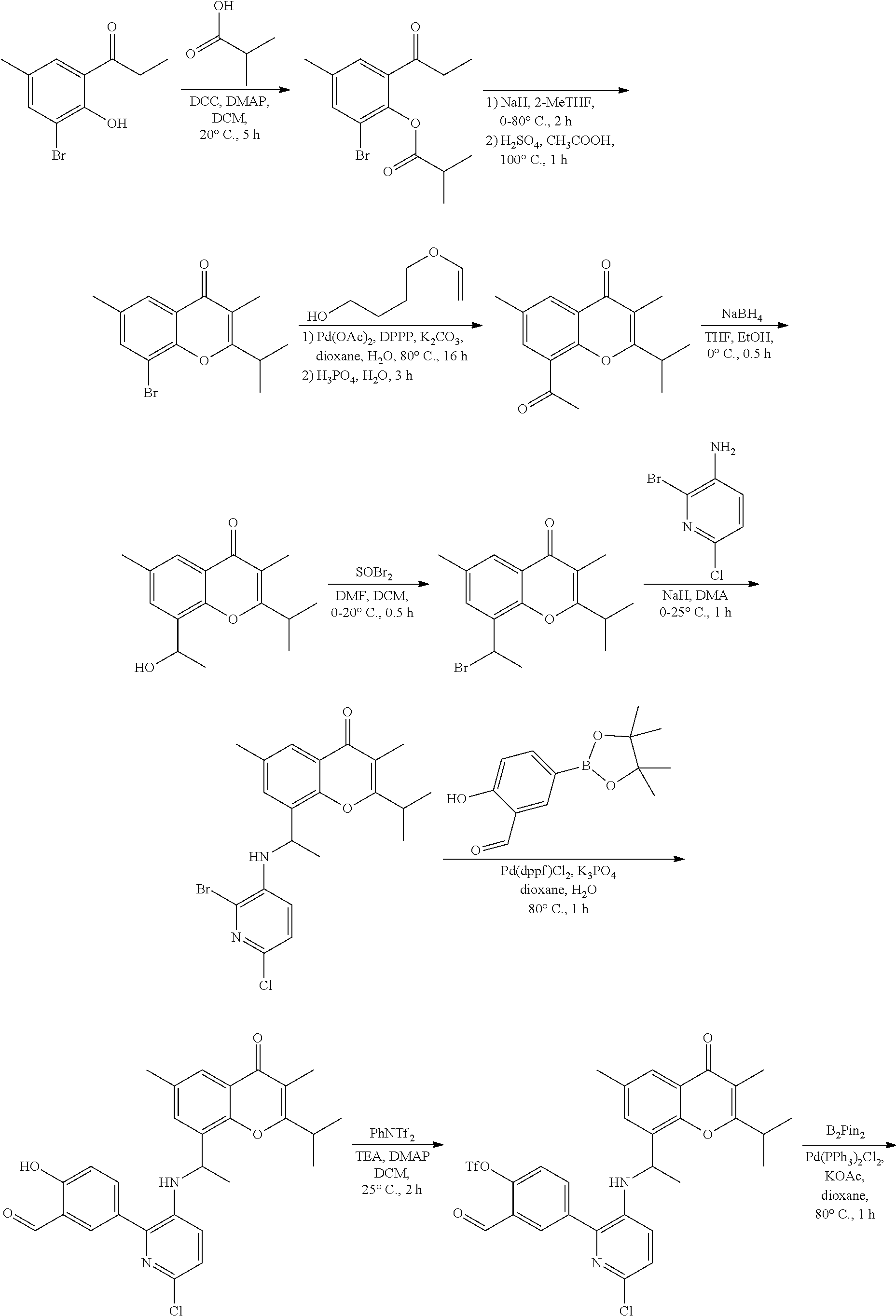

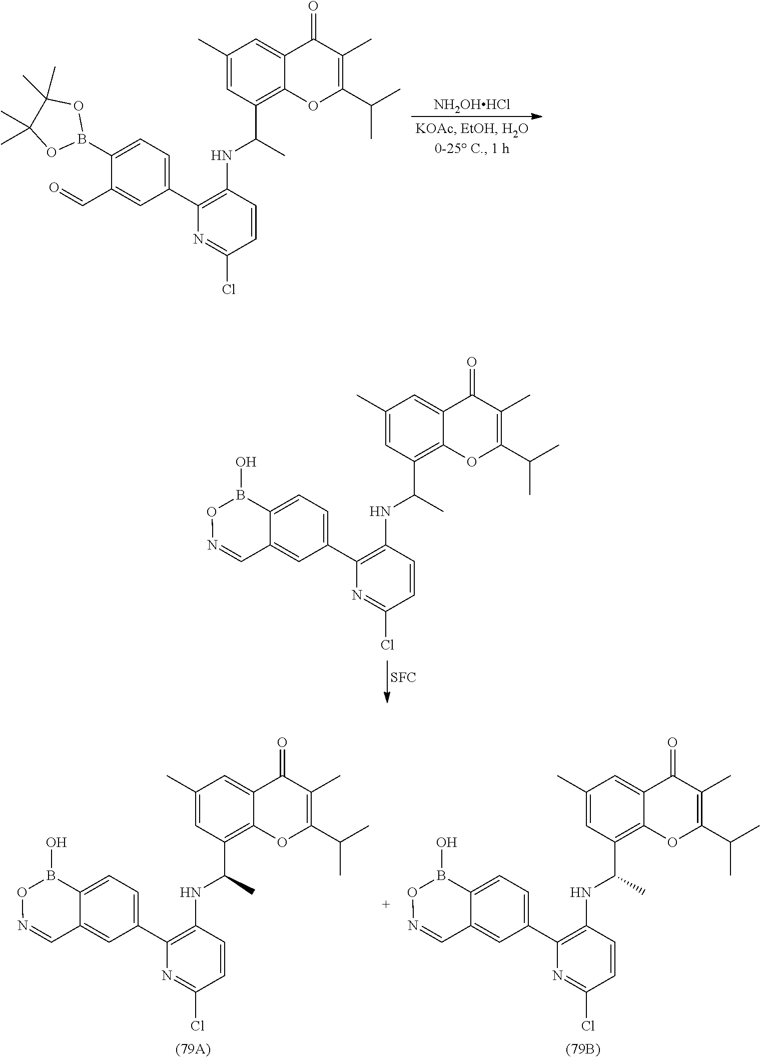

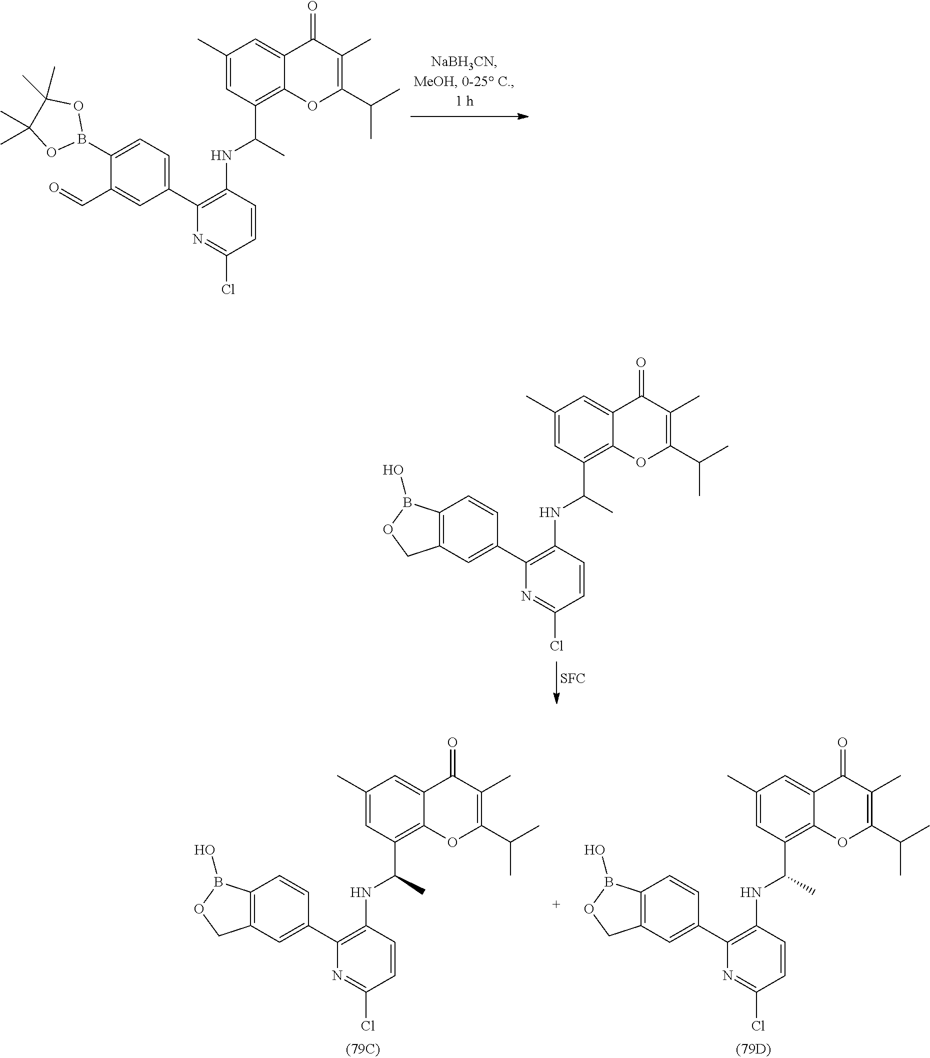

### Preparation of 2-bromo-4-methyl-6-propionylphenyl isobutyrate

To a solution of 1-(3-bromo-2-hydroxy-5-methylphenyl)propan-1-one (10.00 g, 41 mmol, 1 eq) and isobutyric acid (4.35 g, 49 mmol, 4.58 mL, 1.2 eq) in DCM (100 mL) was added DMAP (1.51 g, 12 mmol, 0.3 eq) in portions at 20° C., then a solution of DCC (13.50 g, 65 mmol, 13.3 mL, 1.6 eq) in DCM (100 mL) was added dropwise to the mixture at 20° C. The reaction mixture was stirred at 20° C. for 5 h. The reaction mixture was filtered and the filtrate was concentrated under reduced pressure to give a residue. The residue was purified by flash silica gel chromatography (Biotage®; 120 g SepaFlash® Silica Flash Column, Eluent of 10-35% Ethyl acetate/Petroleum ether gradient @200 mL/min) to give the title compound (12.00 g, 38.3 mmol, 93.14% yield) as a white solid. ¹H NMR (CDCl₃, 400 MHz) δ 7.56 (s, 1H), 7.44 (s, 1H), 2.93-2.83 (m, 3H), 2.37 (s, 3H), 1.37 (d, J=7.2 Hz, 6H), 1.15 (t, J=7.2 Hz, 3H).

### Preparation of 8-bromo-2-isopropyl-3,6-dimethyl-4H-chromen-4-one

To a solution of 2-bromo-4-methyl-6-propionylphenyl isobutyrate (12.00 g, 38.3 mmol, 1 eq) in 2-MeTHF (80 mL) was added NaH (4.60 g, 114 mmol, 3 eq) in portions at 0° C. The mixture was heated to 80° C. and stirred at 80° C. for 2 h. The reaction mixture was cooled to 0° C., quenched by H₂O (200 mL) at 0° C., and then extracted with EtOAc (200 mL×3). The combined organic layers were washed with brine (200 mL), dried over anhydrous Na₂SO₄, filtered and concentrated under reduced pressure to give a residue. To a solution of the residue (13.00 g, 41.5 mmol, 1 eq) in AcOH (65 mL) was added H₂SO₄ (24.4 g, 249 mmol, 13.2 mL, 6 eq) dropwise at 0° C., the reaction mixture was heated to 100° C. and stirred at 100° C. for 1 h. The reaction mixture was cooled to 0° C., quenched with H₂O (300 mL) at 0° C. and filtered. The filter cake was washed with H₂O (10 mL×3) and dried in vacuo to give the title compound (11.00 g, 37.2 mmol, 89.78% yield) as a yellow solid. ¹H NMR (CDCl₃, 400 MHz) δ7.92 (s, 1H), 7.68 (s, 1H), 3.31-3.20 (m, 1H), 2.43 (s, 3H), 2.08 (s, 3H), 1.38 (d, J=6.8 Hz, 6H).

### Preparation of 8-acetyl-2-isopropyl-3,6-dimethyl-4H-chromen-4-one

A mixture of 8-bromo-2-isopropyl-3,6-dimethyl-4H-chromen-4-one (6.00 g, 20.3 mmol, 1 eq), 4-(vinyloxy)butan-1-ol (47.2 g, 406 mmol, 50 mL, 20 eq), DPPP (1.68 g, 4 mmol, 0.2 eq), Pd(OAc)₂ (456 mg, 2.03 mmol, 0.1 eq) and K₂CO₃ (4.21 g, 30.4 mmol, 1.5 eq) in H₂O (50 mL) was degassed and purged with N₂ for 3 times at 20° C., and then the reaction mixture was heated to 80° C. and stirred at 80° C. for 16 h under N₂ atmosphere. The reaction mixture was cooled to 20° C., and then extracted with EtOAc (100 mL×3). The combined organic layers were washed with brine (100 mL), dried over anhydrous Na₂SO₄, filtered and concentrated under reduced pressure to give a residue. Then to the solution of the residue (6.8 M, 35.61 mL, 10 eq) in H₂O (100 mL) was added H₃PO₄ (6.8 M, 35.6 mL, 10 eq) dropwise at 0° C., the reaction mixture was warmed to 20° C. and stirred at 20° C. for 3 h. The reaction mixture was filtered and the filter cake was washed with H₂O (10 mL×3), dried in vacuum to give the title compound (5.00 g, 19.36 mmol, 79.95% yield) as a yellow solid. ¹H NMR (DMSO-d₆, 400 MHz) δ 8.03 (s, 1H), 7.92 (s, 1H), 3.29-3.28 (m, 1H), 2.73 (s, 3H), 2.44 (s, 3H), 2.01 (s, 3H), 1.28 (d, J=7.2 Hz, 6H).

### Preparation of 8-(1-hydroxyethyl)-2-isopropyl-3,6-dimethyl-4H-chromen-4-one

To a solution of 8-acetyl-2-isopropyl-3,6-dimethyl-4H-chromen-4-one (5.00 g, 19.3 mmol, 1 eq) in THF (50 mL)/EtOH (5 mL) was added NaBH₄ (1.10 g, 29.0 mmol, 1.5 eq) in portions at 0° C., the mixture was stirred at 0° C. for 0.5 h. The reaction mixture was quenched by H₂O (100 mL) at 0° C., and then extracted with EtOAc (50 mL×3). The combined organic layers were washed with brine (50 mL), dried over anhydrous Na₂SO₄, filtered and concentrated under reduced pressure to give the title compound (5.00 g, 19.21 mmol, 99.23% yield) as a white solid. ¹H NMR (CDCl₃, 400 MHz) δ 7.89 (s, 1H), 7.62 (s, 1H), 5.48-5.43 (m, 1H), 3.33-3.22 (m, 1H), 2.44 (s, 3H), 2.08 (s, 3H), 1.60-1.58 (m, 3H), 1.34-1.32 (m, 6H).

### Preparation of 8-(1-bromoethyl)-2-isopropyl-3,6-dimethyl-4H-chromen-4-one

To a solution of 8-(1-hydroxyethyl)-2-isopropyl-3,6-dimethyl-4H-chromen-4-one (2.00 g, 7.68 mmol, 1 eq) in DCM (20 mL)/DMF (56 mg, 768 μmol, 59 μL, 0.1 eq) was added SOBr₂ (2.40 g, 11.5 mmol, 892 μL, 1.5 eq) dropwise at 0° C. The mixture was warmed to 20° C. and stirred at 20° C. for 0.5 h. The reaction mixture was quenched by H₂O (20 mL) at 0° C., and then extracted with DCM (30 mL×3). The combined organic layers were dried over anhydrous Na₂SO₄, filtered and concentrated under reduced pressure to give the title compound (2.40 g, 7.43 mmol, 96.65% yield) as a yellow solid. ¹H NMR (DMSO-d₆, 400 MHz) δ 7.96 (s, 1H), 7.58 (s, 1H), 5.71 (dd, J=7.2 Hz 14.0 Hz, 1H), 3.33-3.26 (m, 1H), 2.45 (s, 3H), 2.15 (d, J=7.2 Hz, 3H), 2.09 (s, 3H), 1.42-1.37 (m, 6H).

### Preparation of 8-[1-[(2-bromo-6-chloro-3-pyridyl)amino]ethyl]-2-isopropyl-3,6-dimethyl-chromen-4-one

To a solution of 2-bromo-6-chloro-pyridin-3-amine (1.16 g, 5.57 mmol, 1.2 eq) in DMA (10 mL) was added NaH (279 mg, 6.96 mmol, 1.5 eq) at 0° C. in portions under N₂, the reaction mixture was warmed to 25° C. and stirred at 25° C. for 15 min. Then the reaction mixture was cooled to 0° C. and a solution of 8-(1-bromoethyl)-2-isopropyl-3,6-dimethyl-chromen-4-one (1.5 g, 4.64 mmol, 1 eq) in DMA (10 mL) was added to the reaction mixture dropwise at 0° C. under N₂. The reaction mixture was warmed to 25° C. and stirred at 25° C. for 45 min. The reaction mixture was quenched by addition sat.aq.NH₄Cl (50 mL) at 0° C. and then extracted with EtOAc (20 mL×3). The combined organic phases were washed with brine (15 mL×2), dried over Na₂SO₄, filtered and concentrated under reduced pressure to give a residue. The residue was purified by flash silica gel chromatography (Biotage®; 40 g SepaFlash® Silica Flash Column, Eluent of 0~15% Ethyl acetate/Petroleum ether gradient @60 mL/min) to give the title compound (1.30 g, 2.89 mmol, 62.28% yield) as a light yellow solid. ¹H NMR (CDCl₃, 400 MHz) δ 7.71 (s, 1H), 7.53 (s, 1H), 7.20 (d, J=8.4 Hz, 1H), 6.73 (d, J=8.8 Hz, 1H), 5.84 (d, J=7.2 Hz, 1H), 5.09-5.06 (m, 1H), 2.34 (s, 3H), 2.00 (s, 3H), 1.96 (s, 1H), 1.64 (d, J=6.8 Hz, 3H), 1.30 (dd, J=6.8 Hz, J=2.4 Hz, 6H).

### Preparation of 5-[6-chloro-3-[1-(2-isopropyl-3,6-dimethyl-4-oxo-chromen-8-yl) ethylamino]-2-pyridyl]-2-hydroxy-benzaldehyde

A mixture of 8-[1-[(2-bromo-6-chloro-3-pyridyl)amino]ethyl]-2-isopropyl-3,6-dimethyl-chromen-4-one (1.10 g, 2.45 mmol, 1 eq), 2-hydroxy-5-(4,4,5,5-tetramethyl-1,3,2-dioxaborolan-2-yl)benzaldehyde (606.74 mg, 2.45 mmol, 1 eq), K₃PO₄ (1.04 g, 4.89 mmol, 2 eq) and Pd(dppf)Cl₂ (178.95 mg, 244.57 μmol, 0.1 eq) in dioxane (10 mL)/H₂O (2 mL) was degassed and purged with N₂ for 3 times, and then the reaction mixture was heated to 80° C. and stirred at 80° C. for 1 h under N₂ atmosphere. The reaction mixture was cooled to 25° C. and the organic phase was separated. Then the aqueous phase was diluted with H₂O (15 mL) and extracted with EtOAc (15 mL×3). The combined organic phases were dried over Na₂SO₄, filtered and concentrated under reduced pressure to give a residue. The residue was purified by flash silica gel chromatography (Biotage®; 20 g SepaFlash® Silica Flash Column, Eluent of 0-15% Ethyl acetate/Petroleum ether gradient @45 mL/min) to give the title compound (1.20 g, 2.44 mmol, 99.93% yield) as a light yellow solid. ¹H NMR (CDCl₃, 400 MHz) δ 11.18 (s, 1H), 10.00 (s, 1H), 7.98-7.91 (m, 3H), 7.33 (d, J=2.0 Hz, 1H), 7.16 (d, J=8.8 Hz, 1H), 6.99 (d, J=8.8 Hz, 1H), 6.64 (d, J=8.8 Hz, 1H), 5.00-4.95 (m, 1H), 4.48-4.38 (m, 1H), 3.34-3.27 (m, 1H), 2.39 (s, 3H), 2.11 (s, 3H), 1.57 (d, J=6.4 Hz, 3H), 1.34 (d, J=6.8 Hz, 3H), 1.26 (d, J=6.8 Hz, 3H).

### Preparation of [4-[6-chloro-3-[1-(2-isopropyl-3,6-dimethyl-4-oxo-chromen-8-yl)ethyl amino]-2-pyridyl]-2-formyl-phenyl]trifluoromethanesulfonate

To a solution of 5-[6-chloro-3-[1-(2-isopropyl-3,6-dimethyl-4-oxo-chromen-8-yl)ethylamino]-2-pyridyl]-2-hydroxy-benzaldehyde (1.20 g, 2.44 mmol, 1 eq) and TEA (495 mg, 4.89 mmol, 680.38 μL, 2 eq) in DCM (15 mL) were added 1,1,1-trifluoro-N-phenyl-N-(trifluoromethyl sulfonyl)methanesulfonamide (1.75 g, 4.89 mmol, 2 eq) and DMAP (30 mg, 244.41 μmol, 0.1 eq) in one portion at 25° C., the reaction mixture was stirred at 25° C. for 2 h. The reaction mixture was cooled to 0° C., quenched by adding water (20 mL) and extracted with DCM (15 mL×3). The combined organic layers were dried over Na₂SO₄, filtered and concentrated under reduced pressure to give a residue. The residue was purified by flash silica gel chromatography (Biotage®; 20 g SepaFlash® Silica Flash Column, Eluent of 0~20% Ethyl acetate/Petroleum ether gradient @60 n/min) to give the title compound (1.40 g, 2.25 mmol, 91.94% yield) as a light yellow solid. ¹H NMR (CDCl₃, 400 MHz) δ 10.35 (s, 1H), 8.45 (s, 1H), 8.14 (dd, J=8.0 Hz, J=1.2 Hz, 1H), 7.92 (s, 1H), 7.59 (s, J=8.4 Hz, 1H), 7.36 (d, J=1.6 Hz, 1H), 7.04 (d, J=8.4 Hz, 1H), 6.71 (d, J=8.8 Hz, 1H), 5.03-5.00 (m, 1H), 4.49-4.39 (m, 1H), 3.34-3.27 (m, 1H), 2.41 (s, 3H), 2.11 (s, 3H), 1.60 (s, 3H), 1.34 (d, J=6.8 Hz, 3H), 1.25 (d, J=6.8 Hz, 3H).

### Preparation of 5-[6-chloro-3-[1-(2-isopropyl-3,6-dimethyl-4-oxo-chromen-8-yl)ethyl amino]-2-pyridyl]-2-(4,4,5,5-tetramethyl-1,3,2-dioxaborolan-2-yl)benzaldehyde

A mixture of [4-[6-chloro-3-[1-(2-isopropyl-3,6-dimethyl-4-oxo-chromen-8-yl)ethylamino]-2-pyridyl]-2-formyl-phenyl]trifluoromethanesulfonate (1.40 g, 2.25 mmol, 1 eq), B₂Pin₂ (2.28 g, 8.99 mmol, 4 eq), KOAc (442 mg, 4.49 mmol, 2 eq) and Pd(PPh₃)₂Cl₂ (158 mg, 224.71 μmol, 0.1 eq) in dioxane (15 mL) was degassed and purged with N₂ for 3 times, and then the reaction mixture was heated to 80° C. and stirred at 80° C. for 1 h under N₂ atmosphere. The reaction mixture was cooled to 25° C. and filtered. The filtrate was concentrated under reduced pressure to give a residue. The residue was purified by flash silica gel chromatography (Biotage®; 20 g SepaFlash® Silica Flash Column, Eluent of 0-15% Ethyl acetate/Petroleum ether gradient @45 mL/min) to give the title compound (1.50 g, 2.00 mmol, 88.87% yield) as a yellow solid. ¹H NMR (CDCl₃, 400 MHz) δ 10.62 (s, 1H), 8.35 (s, 1H), 8.05 (d, J=7.6 Hz, 1H), 7.96 (dd, J=7.6 Hz, J=2.0 Hz, 1H), 7.90 (s, 1H), 7.33 (d, J=2.0 Hz, 1H), 6.99 (d, J=8.8 Hz, 1H), 6.62 (d, J=8.8 Hz, 1H), 5.02-4.97 (m, 1H), 4.50-4.42 (m, 1H), 3.33-3.27 (m, 1H), 2.39 (s, 3H), 2.11 (s, 3H), 1.52 (d, J=6.4 Hz, 3H), 1.44 (s, 12H), 1.34 (d, J=6.8 Hz, 3H), 1.29 (d, J=6.8 Hz, 3H).

### Preparation of 8-[1-[[6-chloro-2-(1-hydroxy-2,3,1-benzoxazaborinin-6-yl)-3-pyridyl]amino]ethyl]-2-isopropyl-3,6-dimethyl-chromen-4-one

To a solution of 5-[6-chloro-3-[1-(2-isopropyl-3,6-dimethyl-4-oxo-chromen-8-yl)ethylamino]-2-pyridyl]-2-(4,4,5,5-tetramethyl-1,3,2-dioxaborolan-2-yl)benzaldehyde (600 mg, 798.75 gmol, 1 eq) in EtOH (7 mL)/H₂O (2 mL) was added NH₂OH·HCl (84 mg, 1.20 mmol, 1.5 eq) and KOAc (157 mg, 1.60 mmol, 2 eq) in one portion at 0° C., the mixture was warmed to 25° C. and stirred at 25° C. for 1 h. The reaction mixture diluted with H₂O (10 mL) and the resulting suspension was directly filtered. The filter cake was collected and freeze-dried to give the title compound (300 mg, 581.63 μmol, 72.82% yield) as a white solid. ¹H NMR (CDCl₃, 400 MHz) δ 9.48 (s, 1H), 8.75 (s, 1H), 8.18 (d, J=7.6 Hz, 1H), 8.10 (s, 1H), 8.08 (s, 1H), 7.69 (s, 1H), 7.57 (s, 1H), 7.19 (d, J=8.4 Hz, 1H), 6.90 (d, J=8.4 Hz, 1H), 5.89-5.87 (m, 1H), 5.01-4.98 (m, 1H), 3.29-3.28 (m, 1H), 2.34 (s, 3H), 1.99 (s, 3H), 1.51 (d, J=6.4 Hz, 3H), 1.24 (d, J=7.2 Hz, 3H), 1.17 (d, J=6.8 Hz, 3H).

### Preparation of (S)-8-(1-((6-chloro-2-(1-hydroxy-JH-benzo[d][1,2,6]oxazaborinin-6-yl)pyridin-3-yl)amino)ethyl)-2-isopropyl-3,6-dimethyl-4H-chromen-4-one and (R)-8-(1-((6-chloro-2-(1-hydroxy-1H-benzo[d][1,2,6]oxazaborinin-6-yl)pyridin-3-yl)amino)ethyl)-2-isopropyl-3,6-dimethyl-4H-chromen-4-one

8-[1-[[6-chloro-2-(1-hydroxy-2,3,1-benzoxazaborinin-6-yl)-3-pyridyl]amino]ethyl]-2-isopropyl-3,6-dimethyl-chromen-4-one (300 mg, 581.63 μmol, 1 eq) was purified by SFC (column: ChiralPak IH, 250×30 mm×10 um; mobile phase: [CO2-IPA(0.1% NH₃H₂O)]; B %:30%, isocratic elution mode) to give the title compound, Isomer 1 (104.2 mg, 202.02 μmol, 34.73% yield) as a white solid and the title compound, Isomer 2 (75.5 mg, 146.38 μmol, 25.17% yield) as a white solid. Isomer 1: ¹H NMR (DMSO-d₆, 400 MHz) δ 9.48 (s, 1H), 8.75 (s, 1H), 8.18 (d, J=7.2 Hz, 1H), 8.10-8.08 (m, 2H), 7.68 (s, 1H), 7.57 (d, J=2.0 Hz, 1H), 7.19 (d, J=8.8 Hz, 1H), 6.90 (d, J=8.8 Hz, 1H), 5.88 (d, J=6.8 Hz, 1H), 5.01-4.98 (m, 1H), 3.30-3.27 (m, 1H), 2.34 (s, 3H), 1.98 (s, 3H), 1.51 (d, J=6.8 Hz, 3H), 1.24 (d, J=6.8 Hz, 3H), 1.17 (d, J=6.8 Hz, 3H). MS (ESI): mass calcd. For C₂₈H₂₇BClN₃O₄ 515.18, m/z found 516.4 [M+H]⁺. HPLC: 99.76% (220 nm), 99.79% (254 nm). Chiral purity: 98.44% ee. Isomer 2: ¹H NMR (MeOD, 400 MHz) δ 8.48 (s, 1H), 8.03-7.97 (m, 2H), 7.92 (s, 1H), 7.79 (s, 1H), 7.53 (d, J=2.0 Hz, 1H), 7.10 (d, J=8.8 Hz, 1H), 6.90 (d, J=8.4 Hz, 1H), 5.07-5.05 (m, 1H), 3.39-3.36 (m, 1H), 2.39 (s, 3H), 2.08 (s, 3H), 2.56 (d, J=6.8 Hz, 3H), 1.34 (d, J=6.8 Hz, 3H), 1.26 (d, J=6.8 Hz, 3H). MS (ESI): mass calcd. For C₂₈H₂₇BClN₃O₄ 515.18, m/z found 516.3 [M+H]⁺. HPLC: 98.90% (220 nm), 99.05% (254 nm). Chiral purity: 99.64% ee.

### Preparation of 8-[1-[[6-chloro-2-(1-hydroxy-3H-2,1-benzoxaborol-5-yl)-3-pyridyl]amino]ethyl]-2-isopropyl-3,6-dimethyl-chromen-4-one

To a solution of 5-[6-chloro-3-[1-(2-isopropyl-3,6-dimethyl-4-oxo-chromen-8-yl)ethylamino]-2-pyridyl]-2-(4,4,5,5-tetramethyl-1,3,2-dioxaborolan-2-yl)benzaldehyde (600 mg, 998.44 mol, 1 eq) in MeOH (8 mL) was added NaBH₃CN (95 mg, 1.50 mmol, 1.5 eq) in portions at 0° C. under N₂, then the reaction mixture was warmed to 25° C. and stirred at 25° C. for 1 h under N₂. The reaction mixture was cooled to 0° C., quenched by adding water (5 mL) at 0° C. and then extracted with EtOAc (5 mL×3). The combined organic layers were washed with brine (5 ML), dried over Na₂SO₄, filtered and concentrated under reduced pressure to give a residue. The crude product was triturated with MeCN (5 mL)/H₂O (15 mL) at 25° C. for 10 min and filtered. The filter cake was collected and dried in vacuo to give the title compound (250 mg, 493.67 μmol, 49.44% yield) as a white solid. ¹H NMR (DMSO-d₆, 400 MHz) δ 9.29 (s, 1H), 7.86 (d, J=8.0 Hz, 1H), 7.70-7.69 (m, 3H), 7.56 (d, J=2.0 Hz, 1H), 7.14 (d, J=8.8 Hz, 1H), 6.87 (d, J=8.8 Hz, 1H), 5.56 (d, J=6.8 Hz, 1H), 5.05 (s, 2H), 5.02-4.98 (m, 1H), 3.30-3.26 (m, 1H), 2.34 (s, 3H), 1.98 (s, 3H), 1.52 (d, J=6.4 Hz, 3H), 1.24 (d, J=6.8 Hz, 3H), 1.15 (d, J=6.8 Hz, 3H).

### Preparation of 8-[(1S)-1-[[6-chloro-2-(1-hydroxy-3H-2,1-benzoxaborol-5-yl)-3-pyridyl]amino]ethyl]-2-isopropyl-3,6-dimethyl-chromen-4-one and 8-[(1R)-1-[[6-chloro-2-(1-hydroxy-3H-2,1-benzoxaborol-5-yl)-3-pyridyl]amino]ethyl]-2-isopropyl-3,6-dimethyl-chromen-4-one

8-[1-[[6-chloro-2-(1-hydroxy-3H-2,1-benzoxaborol-5-yl)-3-pyridyl]amino]ethyl]-2-isopropyl-3,6-dimethyl-chromen-4-one (230 mg, 457.44 μmol, 1 eq) was purified by SFC (column: DAICEL CHIRALCEL OD (250 mm×30 mm×10 um); mobile phase: [CO₂-MeOH (0.1% NH₃H₂O)]; B %: 30%, isocratic elution mode) to give the title compound, Isomer 1 (108.4 mg, 214.15 μmol, 46.82% yield) as a white solid and the title compound, Isomer 2 (111.4 mg, 220.66 μmol, 48.24% yield) as a white solid. Isomer 1: ¹H NMR (DMSO-d₆, 400 MHz) δ 9.30 (s, 1H), 7.86 (d, J=8.0 Hz, 1H), 7.70-7.69 (m, 3H), 7.56 (d, J=2.0 Hz, 1H), 7.14 (d, J=8.8 Hz, 1H), 6.86 (d, J=8.8 Hz, 1H), 5.56 (d, J=6.8 Hz, 1H), 5.05 (s, 2H), 5.02-4.98 (m, 1H), 3.30-3.26 (m, 1H), 2.34 (s, 3H), 1.98 (s, 3H), 1.52 (d, J=6.8 Hz, 3H), 1.24 (d, J=6.8 Hz, 3H), 1.15 (d, J=6.8 Hz, 3H). MS (ESI): mass calcd. For C₂₈H₂₈BClN₂O₄ 502.18, m/z found 503.3 [M+H]⁺. HPLC: 99.33% (220 nm), 99.24% (254 nm). Chiral purity: 100% ee. Isomer 2: ¹H NMR (DMSO-d₆, 400 MHz) δ 9.30 (s, 1H), 7.86 (d, J=7.6 Hz, 1H), 7.70-7.69 (m, 3H), 7.56 (d, J=2.0 Hz, 1H), 7.14 (d, J=8.4 Hz, 1H), 6.86 (d, J=8.8 Hz, 1H), 5.58 (d, J=6.8 Hz, 1H), 5.05 (s, 2H), 5.02-4.98 (m, 1H), 3.30-3.26 (m, 1H), 2.34 (s, 3H), 1.98 (s, 3H), 1.52 (d, J=6.8 Hz, 3H), 1.24 (d, J=7.2 Hz, 3H), 1.15 (d, J=6.4 Hz, 3H). MS (ESI): mass calcd. For C₂₈H₂₈BClN₂O₄ 502.18, m/z found 503.3 [M+H]⁺. HPLC: 99.59% (220 nm), 99.86% (254 nm). Chiral purity: 98.28% ee.

### Example 80

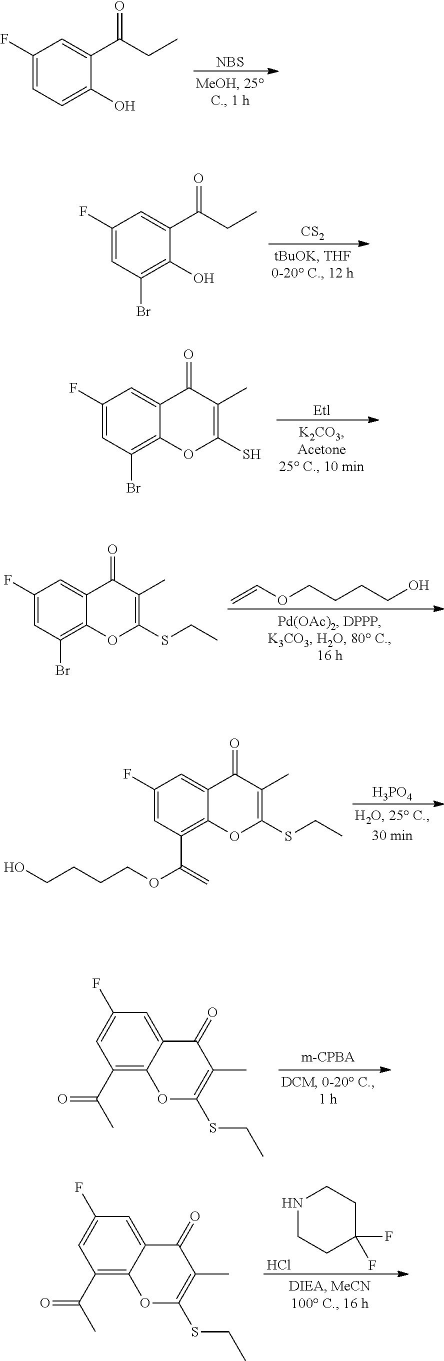

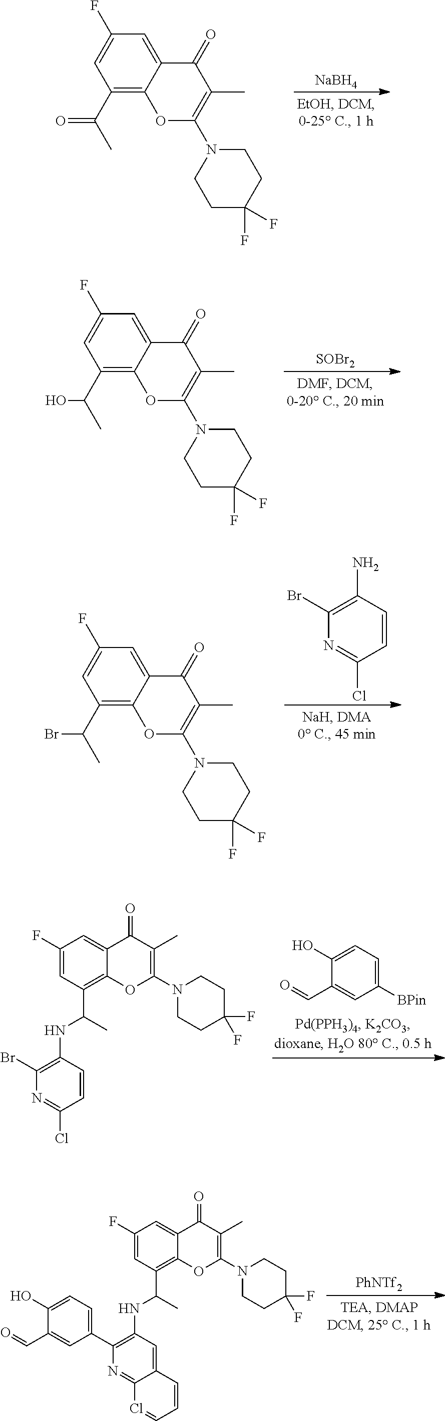

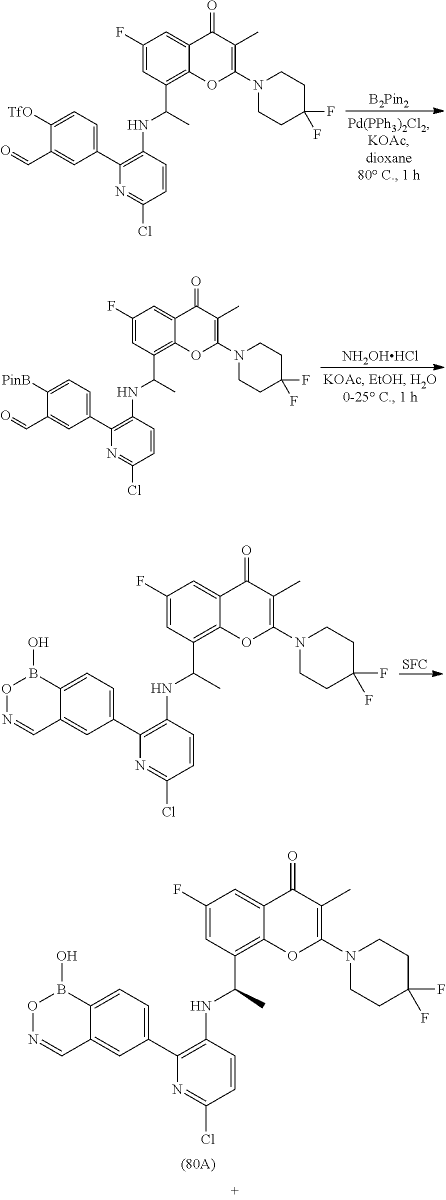

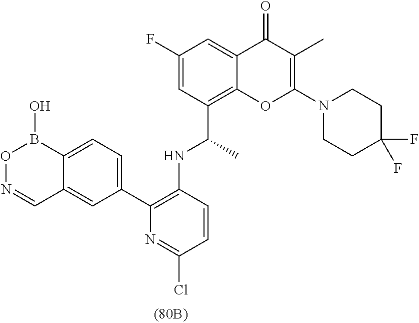 [SMILES: Cc1c(N2CCC(F)(F)CC2)oc2c([C@H](C)Nc3ccc(Cl)nc3-c3ccc4c(c3)C=NOB4O)cc(F)cc2c1=O]

### Preparation of 1-(3-bromo-5-fluoro-2-hydroxy-phenyl)propan-1-one

To a solution of 1-(5-fluoro-2-hydroxy-phenyl)propan-1-one (14.0 g, 83.2 mmol, 1 eq) in MeOH (140 mL) was added NBS (22.2 g, 124 mmol, 1.5 eq) in portions at 25° C., the reaction mixture was stirred at 25° C. for 1 h. The reaction mixture was poured into ice/H₂O (200 mL) and then the solid precipitated out. The resulting suspension was filtered and the filter cake was washed with H₂O (50 mL). The filter cake was dissolved in EtOAc (200 mL), dried over Na₂SO₄, filtered and concentrated under reduced pressure. The crude product was triturated with PE (60 mL) at 25° C. for 20 min and dried in vacuo to give the title compound (12.3 g, 49.7 mmol, 59.8% yield) as a yellow solid. ¹H NMR (DMSO-d₆, 400 MHz) δ 12.53 (s, 1H), 7.93-7.87 (m, 2H), 3.16 (q, J=7.2 Hz, 2H), 1.09 (t, J=7.2 Hz, 3H).

### Preparation of 8-bromo-6-fluoro-3-methyl-2-sulfanyl-chromen-4-one

To a solution of 1-(3-bromo-5-fluoro-2-hydroxy-phenyl)propan-1-one (14.6 g, 59.0 mmol, 1 eq) in THF (180 mL) was added tBuOK (19.8 g, 177 mmol, 3 eq) in portions at 0° C., nitrogen atmosphere protection was applied. After addition, the reaction mixture was stirred at 0° C. for 1 h. Then CS₂ (6.75 g, 88.6 mmol, 5.34 mL, 1.5 eq) was added dropwise to the reaction mixture at 0° C. and the reaction mixture was stirred at 20° C. for 12 h. The reaction mixture was quenched with 6N HCl (40 mL) at 20° C. to pH=2 and extracted with EtOAc (200 mL×3). The combined organic phase was washed with brine (400 mL), dried over Na₂SO₄, filtered and concentrated under reduced pressure. The crude product was triturated with DCM (100 mL) at 20° C. for 60 min and filtered. The filter cake was collected and dried in vacuo give the title compound (15.8 g, 54.6 mmol, 92.4% yield) as a yellow solid. ¹H NMR (DMSO-d₆, 400 MHz) δ 7.99 (dd, J=2.8, 8.4 Hz, 1H), 7.74 (dd, J=2.8, 8.8 Hz, 1H), 2.24 (s, 3H).

### Preparation of 8-bromo-2-ethylsulfanyl-6-fluoro-3-methyl-chromen-4-one

To a solution of 8-bromo-6-fluoro-3-methyl-2-sulfanyl-chromen-4-one (13.6 g, 47.0 mmol, 1 eq) and K₂CO₃ (7.80 g, 56.4 mmol, 1.2 eq) in acetone (150 mL) was added EtI (30.8 g, 197 mmol, 15.8 mL, 4.2 eq) dropwise at 25° C., the reaction mixture was stirred at 25° C. for 10 min. The reaction mixture was quenched with 6N HCl (30 mL) at 20° C. to pH=2, diluted with H₂O (50 mL) and extracted with DCM (200 mL×3). The combined organic phases were washed with brine (200 mL), dried over Na₂SO₄, filtered and concentrated under reduced pressure to give the title compound (14.9 g, 46.9 mmol, 99.8% yield) as a yellow solid. ¹H NMR (CDCl₃, 400 MHz) δ 7.83 (dd, J=2.8, 8.0 Hz, 1H), 7.60 (dd, J=2.8, 7.2 Hz, 1H), 3.33 (q, J=7.6 Hz, 2H), 2.06 (s, 3H), 1.51 (t, J=7.6 Hz, 3H).

### Preparation of 2-ethylsulfanyl-6-fluoro-8-[1-(4-hydroxybutoxy)vinyl]-3-methyl-chromen-4-one

A mixture of 8-bromo-2-ethylsulfanyl-6-fluoro-3-methyl-chromen-4-one (14.9 g, 46.9 mmol, 1 eq), 4-vinyloxybutan-1-ol (109.1 g, 939 mmol, 116.2 mL, 20 eq), K2CO₃ (9.74 g, 70.4 mmol, 1.5 eq) and DPPP (3.88 g, 9.40 mmol, 0.2 eq) in H₂O (100 mL) was added Pd(OAc)₂ (1.05 g, 4.70 mmol, 0.1 eq) in one portion at 20° C., the reaction was degassed and purged with N₂ for 3 times. Then the reaction mixture was directly stirred at 80° C. (the oil-bath was pre-heated to 80° C.) for 16 h under N₂ atmosphere.

The reaction was cooled to 20° C., diluted with H₂O (100 mL) and extracted with EtOAc (150 mL×3).

The combined organic layers were washed with brine (100 mL), dried over Na₂SO₄, filtered and concentrated under reduced pressure to give the title compound (100 g, 45.4 mmol, 96.6% yield) as a brown oil, which was used directly for next step without further purification.

### Preparation of 8-acetyl-2-ethylsulfanyl-6-fluoro-3-methyl-chromen-4-one

To a solution of 2-ethylsulfanyl-6-fluoro-8-[1-(4-hydroxybutoxy)vinyl]-3-methyl-chromen-4-one (100 g, 45.4 mmol, 1 eq) in H₂O (116 mL) was added dropwise H₃PO₄ (195 g, 1.99 mol, 116 mL, 43.8 eq) dropwise at 0° C., the reaction mixture was stirred at 25° C. for 30 mn. The solid was precipitated out. The resulting suspension was filtered and the filter cake was washed with H₂O (50 mL). The filter cake was dissolved in EtOAc (200 mL), dried over Na₂SO₄, filtered and concentrated under reduced pressure. The crude product was triturated with MTBE (50 mL) at 25° C. for 30 min and filtered. The filter cake was collected and dried in vacuo to give the title compound (12 g, 42.8 mmol, 94.2% yield) as a yellow solid. ¹H NMR (DMSO-d₆, 400 MHz) δ 8.11 (dd, J=2.8, 8.4 Hz, 1H), 7.90 (dd, J=2.8, 7.6 Hz, 1H), 3.28 (t, J=7.2 Hz, 2H), 2.70 (s, 3H), 1.95 (s, 3H), 1.34 (t, J=7.2 Hz, 3H).

### Preparation of 8-acetyl-2-ethylsulfinyl-6-fluoro-3-methyl-chromen-4-one

To a solution of 8-acetyl-2-ethylsulfanyl-6-fluoro-3-methyl-chromen-4-one (10.00 g, 35.6 mmol, 1 eq) in DCM (150 mL) was added m-CPBA (9.42 g, 46.3 mmol, 1.3 eq) in portions at 0° C. under N₂, the reaction mixture was stirred at 20° C. for 1 h. The reaction mixture was quenched by addition sat. aq. Na₂SO₃ at 0° C. until the KI testing paper doesn't turn blue. Then the mixture was diluted with H₂O (200 mL) and extracted with DCM (200 mL×3). The combined organic layers were washed with brine (200 mL), dried over Na₂SO₄, filtered and concentrated under reduced pressure. The crude product was triturated with MTBE (50 mL) at 20° C. for 20 min and filtered. The filter cake was collected and dried in vacuo to give the title compound (8.50 g, 28.6 mmol, 80.4% yield) as a yellow solid. ¹H NMR (DMSO-d₆, 400 MHz) δ 8.03-7.97 (m, 2H), 3.27 (q, J=7.2 Hz, 2H), 2.82 (s, 3H), 2.09 (s, 3H), 1.23 (t, J=7.2 Hz, 3H).

### Preparation of 8-acetyl-2-(4,4-difluoro-1-piperidyl)-6-fluoro-3-methyl-chromen-4-one

To a solution of 8-acetyl-2-ethylsulfinyl-6-fluoro-3-methyl-chromen-4-one (3.50 g, 11.8 mmol, 1 eq) in MeCN (40 mL) were added DIEA (9.16 g, 70.8 mmol, 12.3 mL, 6 eq) and 4,4-difluoropiperidine;hydrochloride (5.58 g, 35.4 mmol. 3 eq) in turns at 25° C., then the reaction mixture was directly stirred at 100° C. (the oil-bath was pre-heated to 100° C.) for 16 h. The reaction was cooled to 20° C. and directly concentrated under reduced pressure to give a residue. The residue was purified by flash silica gel chromatography (BIOTAGE®; 80 g SepaFlash® Silica Flash Column, Eluent of 0~50% Ethyl acetate/Petroleum ether gradient @100 mL/min) to give the title compound (2.20 g, 6.48 mmol, 54.89% yield) as a yellow solid. ¹H NMR (DMSO-d₆, 400 MHz) δ 7.98 (dd, J=3.2, 8.8 Hz, 1H), 7.86 (dd, J=3.6, 8.0 Hz, 1H), 3.58-3.55 (m, 4H), 2.71 (s, 3H), 2.24-2.09 (m, 4H), 1.96 (s, 3H).

### Preparation of 2-(4,4-difluoro-1-piperidyl)-6-fluoro-8-(1-hydroxyethyl)-3-methyl-chromen-4-one

To a solution of 8-acetyl-2-(4,4-difluoro-1-piperidyl)-6-fluoro-3-methyl-chromen-4-one (2.2 g, 6.48 mmol, 1 eq) in EtOH (15 mL)/DCM (15 mL) was added NaBH₄ (539 mg, 14.2 mmol, 2.2 eq) in portions at 0° C., the reaction mixture was stirred at 25° C. for 1 h. The reaction mixture was poured into ice water (50 mL) at 0° C. and then extracted with DCM (50 mL×3). The combined organic phase was washed with brine (50 mL), dried over Na₂SO₄, filtered and concentrated under reduced pressure to give the title compound (2.2 g, 6.45 mmol, 99.4% yield) as a yellow solid. ¹H NMR (DMSO-d₆, 400 MHz) δ 7.56 (dd, J=2.8, 9.2 Hz, 1H), 7.50 (dd, J=3.2, 8.0 Hz, 1H), 5.52 (d, J=4.4 Hz, 1H), 5.25-5.20 (m, 1H), 3.54-3.51 (m, 4H), 2.26-2.05 (m, 4H), 1.94 (s, 3H), 1.43 (d, J=6.4 Hz, 3H).

### Preparation of 8-(1-bromoethyl)-2-(4,4-difluoro-1-piperidyl)-6-fluoro-3-methyl-chromen-4-one

To a solution of 2-(4,4-difluoro-1-piperidyl)-6-fluoro-8-(1-hydroxyethyl)-3-methyl-chromen-4-one (1.50 g, 4.39 mmol, 1 eq) in DCM (20 mL)/DMF (3.21 mg, 43.9 gmol, 3.38 μL, 0.01 eq) was added SOBr₂ (1.37 g, 6.59 mmol, 510 μL, 1.5 eq) dropwise at 0° C., the reaction mixture was warmed to 20° C. and stirred 20° C. for 20 min. The reaction mixture was poured into ice/H₂O (50 mL) at 0° C. and extracted with DCM (50 mL×3). The combined organic layers were washed with sat.aq NaHCO₃ until pH=8, then washed with brine (50 mL), dried over Na₂SO₄, filtered and concentrated under reduced pressure to give the title compound (1.75 g, 4.33 mmol, 98.51% yield) as a yellow solid. ¹H NMR (DMSO-d₆ 400 MHz) δ 7.85 (dd, J=3.2, 9.6 Hz, 1H), 7.63 (dd, J=3.2, 8.4 Hz, 1H), 5.90-5.84 (m, 1H), 3.63-3.60 (m, 4H), 2.23-2.14 (m, 4H), 2.11 (d, J=6.8 Hz, 3H), 1.95 (s, 3H).

### Preparation of 8-[1-[(2-bromo-6-chloro-3-pyridyl)amino]ethyl]-2-(4,4-difluoro-1-piperidyl)-6-fluoro-3-methyl-chromen-4-one

To a solution of 2-bromo-6-chloro-pyridin-3-amine (1.03 g, 4.98 mmol, 1.15 eq) in DMA (10 mL) was added NaH (259 mg, 6.49 mmol, 6.25 μL, 1.5 eq) in portions at 0° C., the reaction mixture was stirred at 0° C. for 15 min. Then to the reaction was added dropwise a solution of 8-(1-bromoethyl)-2-(4,4-difluoro-1-piperidyl)-6-fluoro-3-methyl-chromen-4-one (1.75 g, 4.33 mmol, 1 eq) in DMA (10 mL) at 0° C. and the resulting reaction mixture was stirred at 0° C. for 30 min. The reaction mixture was quenched with H₂O (50 mL) at 0° C. and extracted with EtOAc (50 mL×3). The combined organic layers were washed with brine (50 mL), dried over Na₂SO₄, filtered and concentrated under reduced pressure to give a residue. The residue was purified by flash silica gel chromatography (BIOTAGE®; 40 g SepaFlash® Silica Flash Column, Eluent of 0~50% Ethyl acetate/Petroleum ether gradient @80 mL/min) to give the title compound (1.40 g, 2.64 mmol, 60.93% yield) as yellow oil. ¹H NMR (DMSO-d₆, 400 MHz) δ 7.52 (dd, J=3.2, 8.4 Hz, 1H), 7.44 (dd, J=3.2, 9.6 Hz, 1H), 7.17 (d, J=8.8 Hz, 1H), 6.82 (d, J=8.4 Hz, 1H), 5.91 (d, J=7.6 Hz, 1H), 5.07-5.01 (m, 1H), 3.58-3.53 (m, 4H), 2.21-2.10 (m, 4H), 1.96 (s, 3H), 1.65 (d, J=6.8 Hz, 3H).

### Preparation of 5-[6-chloro-3-[1-[2-(4,4-difluoro-1-piperidyl)-6-fluoro-3-methyl-4-oxo-chromen-8-yl]ethylamino]-2-pyridyl]-2-hydroxy-benzaldehyde

A mixture of 8-[I-[(2-bromo-6-chloro-3-pyridyl)amino]ethyl]-2-(4,4-difluoro-1-piperidyl)-6-fluoro-3-methyl-chromen-4-one (500 mg, 942 μmol, 1 eq), 2-hydroxy-5-(4,4,5,5-tetramethyl-1,3,2-dioxaborolan-2-yl)benzaldehyde (303 mg, 1.22 mmol, 1.3 eq) and K₂CO₃ (351 mg, 2.54 mmol, 2.7 eq) in dioxane (5 mL)/H₂O (1 mL) was added Pd(PPh₃)₄ (108 mg, 94.2 μmol, 0.1 eq) in one portion at 20° C., the reaction was degassed and purged with N₂ for 3 times. Then the reaction mixture was directly stirred at 80° C. (the oil-bath was pre-heated to 80° C.) for 0.5 h under N₂ atmosphere. The reaction mixture was cooled to 20° C. and filtered. The filtrate was concentrated under reduced pressure and the residue was purified by flash silica gel chromatography (BIOTAGE®; 20 g SepaFlash® Silica Flash Column, Eluent of 0~100% Ethyl acetate/Petroleum ether gradient @60 mL/min) to give the title compound (450 mg, crude) as a yellow solid, which was used directly for next step without further purification.

### Preparation of [4-[6-chloro-3-[1-[2-(4,4-difluoro-1-piperidyl)-6-fluoro-3-methyl-4-oxo-chromen-8-yl]ethylamino]-2-pyridyl]-2-formyl-phenyl]trifluoromethanesulfonate

To a mixture of 5-[6-chloro-3-[1-[2-(4,4-difluoro-1-piperidyl)-6-fluoro-3-methyl-4-oxo-chromen-8-yl]ethylamino]-2-pyridyl]-2-hydroxy-benzaldehyde (450 mg, 786 μmol, 1 eq), TEA (159 mg, 1.57 mmol, 2 eq) and DMAP (9.61 mg, 78.6 μmol, 0.1 eq) in DCM (5 mL) was added 1,1,1-trifluoro-N-phenyl-N-(trifluoromethylsulfonyl)methanesulfonamide (365 mg, 1.02 mmol, 1.3 eq) in portions at 25° C., the reaction mixture was directly stirred at 25° C. for 1 h. The reaction mixture was partitioned between H₂O (20 mL) and DCM (50 mL). The organic phase was separated, washed with brine (20 mL), dried over Na₂SO₄, filtered and concentrated under reduced pressure to give a residue. The residue was purified by flash silica gel chromatography (BIOTAGE®; 12 g SepaFlash® Silica Flash Column, Eluent of 0~50% Ethyl acetate/Petroleum ether gradient @60 mL/min) to give the title compound (450 mg, 639 gmol, 81.2% yield) as a yellow solid. ¹H NMR (DMSO-d₆, 400 MHz) δ 10.17 (s, 1H), 8.50 (d, J=2.4 Hz, 1H), 8.29 (dd, J=2.4, 8.8 Hz, 1H), 7.73 (d, J=8.8 Hz, 1H), 7.59-7.45 (m, 2H), 7.16 (d, J=8.8 Hz, 1H), 6.91 (d, J=8.8 Hz, 1H), 6.20 (d, J=7.2 Hz, 1H), 5.07-4.93 (m, 1H), 3.57-3.46 (m, 4H), 2.20-2.11 (m, 4H), 1.98-1.88 (m, 3H), 1.61-1.52 (m, 3H).

### Preparation of 5-[6-chloro-3-[1-[2-(4,4-difluoro-1-piperidyl)-6-fluoro-3-methyl-4-oxo-chromen-8-yl]ethylamino]-2-pyridyl]-2-(4,4,5,5-tetramethyl-1,3,2-dioxaborolan-2-yl)benzaldehyde

A mixture of [4-[6-chloro-3-[1-[2-(4,4-difluoro-1-piperidyl)-6-fluoro-3-methyl-4-oxo-chromen-8-yl]ethylamino]-2-pyridyl]-2-formyl-phenyl]trifluoromethanesulfonate (450 mg, 639 μmol, 1 eq), 4,4,5,5-tetramethyl-2-(4,4,5,5-tetramethyl-1,3,2-dioxaborolan-2-yl)-1,3,2-dioxabo rolane (649 mg, 2.56 mmol, 4 eq), KOAc (125 mg, 1.28 mmol, 2 eq) and Pd(PPh₃)₂Cl₂ (44.8 mg, 63.9 μmol, 0.1 eq) in dioxane (5 mL) was degassed and purged with N₂ for 3 times. Then the reaction mixture was directly stirred at 80° C. (the oil-bath was pre-heated to 80° C.) for 1 h under N₂ atmosphere. The reaction was cooled to 20° C. and filtered. The filtrate was directly concentrated under reduced pressure and the crude product was triturated with MTBE (10 ml) at 20° C. for 20 min to give the title compound (0.7 g, crude) as a yellow solid, which was used directly for next step without further purification.

### Preparation of 8-[1-[[6-chloro-2-(1-hydroxy-2,3,1-benzoxazaborinin-6-yl)-3-pyridyl]amino]ethyl]-2-(4,4-difluoro-1-piperidyl)-6-fluoro-3-methyl-chromen-4-one

To a mixture of 5-[6-chloro-3-[1-[2-(4,4-difluoro-1-piperidyl)-6-fluoro-3-methyl-4-oxo-chromen-8-yl]ethylamino]-2-pyridyl]-2-(4,4,5,5-tetramethyl-1,3,2-dioxaborolan-2-yl)benzaldehyde (170.0 mg, 249 μmol, 1 eq) in EtOH (1 mL)/H₂O (1 mL) were added NH₂OH·HCl (17.3 mg, 249 μmol, 1 eq) and KOAc (24.4 mg, 249 μmol, 1 eq) in turns at 20° C., the reaction mixture was directly stirred at 50° C. (the oil-bath was pre-heated to 50° C.) for 1 h. The reaction mixture was cooled to 20° C. and filtered. The filter cake was purified by prep-HPLC (column: Waters Xbridge Prep OBD C18 150×40 mm×10 um; mobile phase: [H₂O (10 mM NH₄HCO₃)-AC□]; gradient: 15%-55% B over 8.0 min) to give the title compound (30 mg, 50.2 gmol, 20.1% yield) as a white solid. ¹H NMR (DMSO-d₆, 400 MHz) δ 9.49 (s, 1H), 8.73 (s, 1H), 8.17 (d, J=8.0 Hz, 1H), 8.09-8.06 (m, 1H), 7.53-7.44 (m, 2H), 7.16 (d, J=8.8 Hz, 1H), 6.95 (d, J=8.8 Hz, 1H), 6.53 (s, 1H), 5.95 (d, J=6.8 Hz, 1H), 5.97-4.86 (m, 1H), 3.53-3.49 (m, 4H), 2.19-2.08 (m, 4H), 1.94 (s, 3H), 1.52 (d, J=6.4 Hz, 3H).

### Preparation of 8-[(1S)-1-[[6-chloro-2-(1-hydroxy-2,3,1-benzoxazaborinin-6-yl)-3-pyridyl]amino]ethyl]-2-(4,4-difluoro-1-piperidyl)-6-fluoro-3-methyl-chromen-4-one and 8-[(1R)-1-[[6-chloro-2-(1-hydroxy-2,3,1-benzoxazaborinin-6-yl)-3-pyridyl]amino]ethyl]-2-(4,4-difluoro-1-piperidyl)-6-fluoro-3-methyl-chromen-4-one

8-[1-[[6-chloro-2-(1-hydroxy-2,3,1-benzoxazaborinin-6-yl)-3-pyridyl]amino]ethyl]-2-(4,4-difluoro-1-piperidyl)-6-fluoro-3-methyl-chromen-4-one (60 mg) was purified by SFC (column: ChiralPak IH, 250×30 mm×10 um; mobile phase: [CO₂-EtOH]; B %: 40%, isocratic elution mode) to give the title compound, Isomer 1 (15.1 mg, 25.3 gmol, 25.1% yield) as a white solid and the title compound, Isomer 2 (16 mg, 26.8 μmol, 26.6% yield) as a white solid. Isomer 1: ¹H NMR (DMSO-d₆, 400 MHz) δ 9.48 (s, 1H), 8.74 (s, 1H), 8.17 (d, J=8.4 Hz, 1H), 8.13-8.07 (m, 2H), 7.51-7.47 (m, 2H), 7.16 (d, J=8.8 Hz, 1H), 6.95 (d, J=8.8 Hz, 1H), 5.97 (d, J=6.8 Hz, 1H), 4.97-4.92 (m, 1H), 3.50 (t, J=5.6 Hz, 4H), 2.18-2.09 (m, 4H), 1.95 (s, 3H), 1.53 (d, J=6.4 Hz, 3H). MS (ESI): mass calcd. For C₂₉H₂₅BClF₃N₄O₄ 596.16, m/z found 597.1 [M+H]⁺. HPLC: 98.77% (220 nm), 99.16% (254 nm). Chiral purity: 100% ee. Isomer 2: ¹H NMR (DMSO-d₆, 400 MHz) δ 9.48 (s, 1H), 8.74 (s, 1H), 8.17 (d, J=8.4 Hz, 1H), 8.10-8.07 (m, 2H), 7.51-7.46 (m, 2H), 7.16 (d, J=8.8 Hz, 1H), 6.95 (d, J=8.4 Hz, 1H), 5.97 (d, J=7.2 Hz, 1H), 4.94 (t, J=7.2 Hz, 1H), 3.50 (t, J=5.6 Hz, 4H), 2.19-2.10 (m, 4H), 1.95 (s, 3H), 1.53 (d, J=6.4 Hz, 3H). MS (ESI): mass calcd. For C₂₉H₂₅BClF₃N₄O₄ 596.16, m/z found 597.1 [M+H]⁺. HPLC: 98.60% (220 nm), 98.55% (254 nm). Chiral purity: 99.32% ee

### Example 81

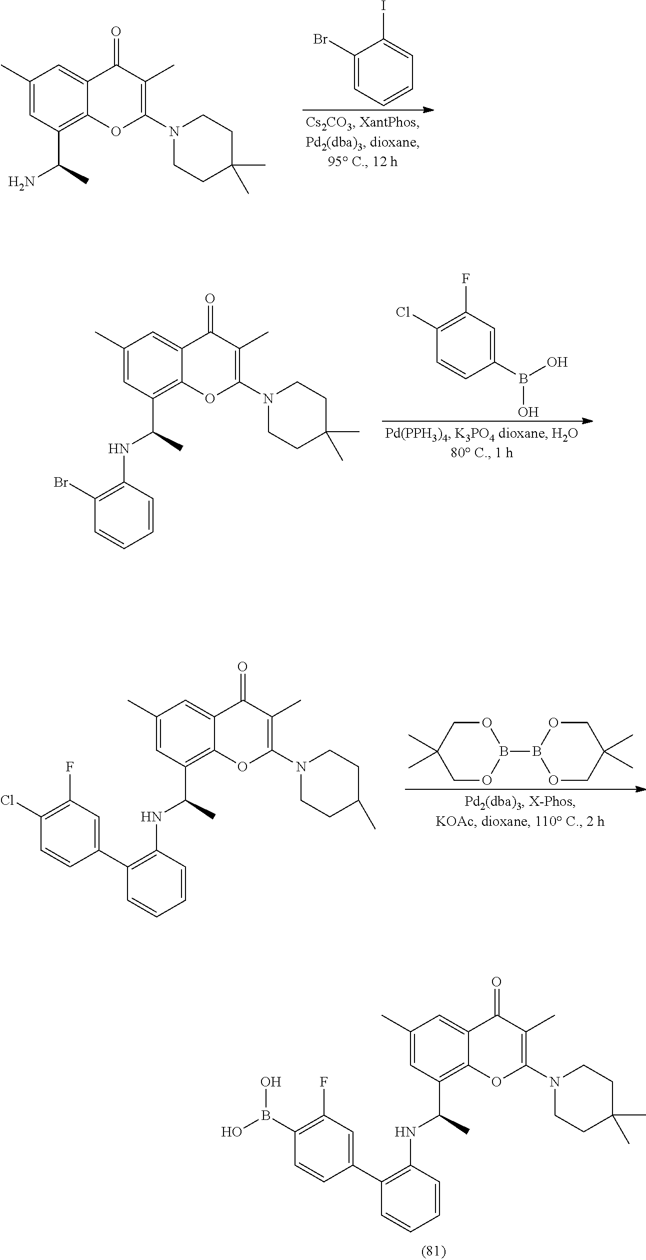

### Preparation of 8-[(1R)-1-(2-bromoanilino)ethyl]-2-(4,4-dinmethyl-1-piperidyl)-3,6-dimethyl-chromen-4-one

The title compound was prepared as a light yellow oil (1.40 g, 2.90 mmol, 95.12% yield) according to the similar procedure in Example 78, using 8-[(1R)-1-aminoethyl]-2-(4,4-dimethyl-1-piperidyl)-3,6-dimethyl-chromen-4-one (1.00 g, 3.04 mmol, 1 eq) and 1-bromo-2-iodo-benzene (1.72 g, 6.09 mmol, 781.97 μL, 2 eq). ¹H NMR (CDCl₃, 400 MHz) δ 7.87 (s, 1H), 7.44 (dd, J=8.0 Hz, J=3.2 Hz, 1H), 7.38 (d, J=2.0 Hz, 1H), 7.04-7.00 (m, 1H), 6.57-6.53 (m, 1H), 6.33 (dd, J=8.4 Hz, J=1.2 Hz, 1H), 5.00-4.99 (m, 1H), 4.74 (s, 1H), 3.42-3.39 (m, 4H), 2.37 (s, 3H), 2.06 (s, 3H), 1.66 (d, J=6.8 Hz, 3H), 1.54-1.51 (m, 4H), 1.04 (s, 6H).

### Preparation of 8-[(1R)-1-[2-(4-chloro-3-fluoro-phenyl)anilino]ethyl]-2-(4,4-dimethyl-1-piperidyl)-3,6-dimethyl-chromen-4-one

A mixture of 8-[(1R)-1-(2-bromoanilino)ethyl]-2-(4,4-dimethyl-1-piperidyl)-3,6-dimethyl-chromen-4-one (300 mg, 620.55 μmol, 1 eq), (4-chloro-3-fluoro-phenyl)boronic acid (119 mg, 682.61 μmol, 1.1 eq), K₃PO₄ (263 mg, 1.24 mmol, 2 eq) and Pd(dppf)Cl₂ (46 mg, 62.06 μmol, 0.1 eq) in H₂O (1 mL)/dioxane (5 mL) was degassed and purged with N₂ for 3 times, and then the reaction mixture was heated to 80° C. and stirred at 80° C. for 1 h under N₂ atmosphere. The reaction mixture was cooled to 25° C., diluted with H₂O (10 mL) and extracted with EtOAc (10 mL×3). The combined organic layers were dried over Na₂SO₄, filtered and concentrated under reduced pressure to give a residue. The residue was purified by flash silica gel chromatography (Biotage®; 12 g SepaFlash® Silica Flash Column, Eluent of 0~18% Ethyl acetate/Petroleum ether gradient @50 mL/min). The eluent was removed under reduced pressure to give the title compound (280 mg, 525.25 μmol, 84.64% yield) as a light yellow solid. ¹H NMR (CDCl₃, 400 MHz) δ 7.90 (s, 1H), 7.51 (t, J=8.0 Hz, 1H), 7.33-7.29 (m, 2H), 7.24-7.22 (m, 1H), 7.11-7.06 (m, 2H), 6.76 (t, J=7.2 Hz, 1H), 6.40 (d, J=8.0 Hz, 1H), 4.99-4.94 (m, 1H), 3.38-3.37 (m, 4H), 2.37 (s, 3H), 2.08 (s, 3H), 1.52-1.49 (m, 7H), 1.03 (s, 6H).

### Preparation of [4-[2-[[(1R)-1-[2-(4,4-dimethyl-1-piperidyl)-3,6-dimethyl-4-oxo-chromen-8-yl]ethyl]amino]phenyl]-2-fluoro-phenyl]boronic acid

A mixture of 8-[(1R)-1-[2-(4-chloro-3-fluoro-phenyl)anilino]ethyl]-2-(4,4-dimethyl-1-piperidyl)-3,6-dimethyl-chromen-4-one (280 mg, 525.25 μmol, 1 eq), 2-(5,5-dimethyl-1,3,2-dioxaborinan-2-yl)-5,5-dimethyl-1,3,2-dioxaborinane (475 mg, 2.10 mmol, 4 eq), KOAc (155 mg, 1.58 mmol, 3 eq), Pd₂(dba)₃ (48 mg, 52.53 μmol, 0.1 eq) and XPhos (25 mg, 52.53 μmol, 0.1 eq) in dioxane (5 mL) was degassed and purged with N₂ for 3 times, and then the reaction mixture was heated to 110° C. and stirred at 110° C. for 2 h under N₂ atmosphere. The reaction mixture was cooled to 25° C. and filtered. The filtrate was concentrated under reduced pressure and the residue was purified by prep-HPLC (column: Waters Xbridge Prep OBD C18 150×40 mm×10 um; mobile phase: [H₂O (10 mM NH₄HCO₃)-AC□]; gradient: 55%-85% B over 8.0 min) to give the title compound (106.2 mg, 195.78 μmol, 37.27% yield) as a light yellow solid. ¹H NMR (DMSO-d₆, 400 MHz) δ 8.21 (s, 2H), 7.68 (t, J=6.8 Hz, 1H), 7.62 (s, 1H), 7.45 (d, J=2.0 Hz, 1H), 7.28 (d, J=7.6 Hz, 1H), 7.18 (d, J=10.0 Hz, 1H), 7.05-7.00 (m, 2H), 6.67 (t, J=7.2 Hz, 1H), 6.47 (d, J=8.0 Hz, 1H), 5.00-4.95 (m, 1H), 4.85 (d, J=6.4 Hz, 1H), 3.34-3.29 (m, 4H), 2.32 (s, 3H), 1.91 (s, 3H), 1.47 (d, J=6.4 Hz, 3H), 1.44-1.42 (m, 4H), 0.97 (s, 6H). MS (ESI): mass calcd. For C₃₂H₃₆BFN₂O₄ 542.28, m/z found 543.4 [M+H]⁺. HPLC: 97.65% (220 nm), 99.28% (254 nm). Chiral purity: 98.08% ee.

### Example 82

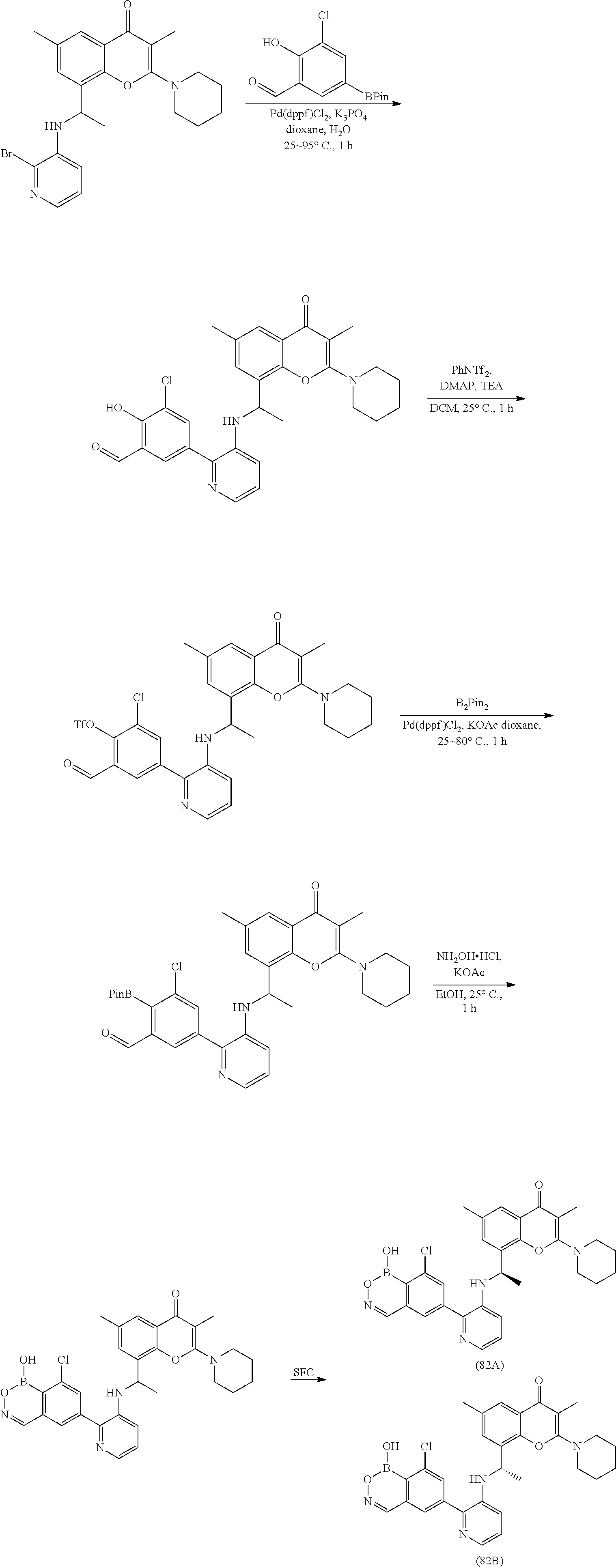

### Preparation of 3-chloro-5-[3-[1-[3,6-dimethyl-4-oxo-2-(1-piperidyl)chromen-8-yl]ethyl amino]-2-pyridyl]-2-hydroxy-benzaldehyde

The title compound was prepared as a yellow solid (1.30 g, crude) according to the procedures described herein and the synthetic scheme, using 8-[1-[(2-bromo-3-pyridyl)amino]ethyl]-3,6-dimethyl-2-(1-piperidyl)chromen-4-one (1.00 g, 2.19 mmol, 1 eq) and 3-chloro-2-hydroxy-5-(4,4,5,5-tetramethyl-1,3,2-dioxaborolan-2-yl)benzaldehyde (619 mg, 2.19 mmol, 1 eq). ¹H NMR (DMSO-d₆, 400 MHz) δ 11.31 (s, 1H), 10.20 (s, 1H), 8.12 (d, J=2.0 Hz, 1H), 8.06 (d, J=2.4 Hz, 1H), 7.91 (d, J=3.2 Hz, 1H), 7.61 (d, J=2.0 Hz, 1H), 7.49 (d, J=2.0 Hz, 1H), 7.06-7.05 (m, 1H), 6.89-6.87 (m, 1H), 5.69 (d, J=7.6 Hz, 1H), 4.94 (t, J=6.8 Hz, 1H), 3.28 (s, 4H), 2.32 (s, 3H), 1.90 (s, 3H), 1.60 (s, 6H), 1.50 (d, J=6.8 Hz, 3H).

### Preparation of [2-chloro-4-[3-[1-[3,6-dimethyl-4-oxo-2-(1-piperidyl)chromen-8-yl]ethyl amino]-2-pyridyl]-6-formyl-phenyl]trifluoromethanesulfonate

The title compound was prepared as a yellow gum (1.10 g, 1.49 mmol, 79.3% yield) according to the procedures described herein and the synthetic scheme, using 3-chloro-5-[3-[1-[3,6-dimethyl-4-oxo-2-(1-piperidyl)chromen-8-yl]ethylamino]-2-pyridyl]-2-hydroxy-benzaldehyde (1.00 g, 1.88 mmol, 1 eq) and PhNTf₂ (1.34 g, 3.76 mmol, 2 eq). ¹H NMR (CDCl₃, 400 MHz) δ 10.28 (s, 1H), 8.38 (s, 1H), 8.25 (s, 1H), 8.06 (d, J=4.4 Hz, 1H), 7.89 (s, 1H), 7.33 (s, 1H), 7.08-7.05 (m, 1H), 6.75-6.72 (m, 1H), 4.98 (t, J=6.8 Hz, 1H), 4.42 (d, J=4.4 Hz, 1H), 3.36 (s, 4H), 2.38 (s, 3H), 2.06 (s, 3H), 1.70 (s, 6H), 1.61 (d, J=6.8 Hz, 3H).

### Preparation of 3-chloro-5-[3-[1-[3,6-dimethyl-4-oxo-2-(1-piperidyl)chromen-8-yl]ethyl amino]-2-pyridyl]-2-(4,4,5,5-tetramethyl-1,3,2-dioxaborolan-2-yl)benzaldehyde

The title compound was prepared as a light-yellow solid (520 mg, 663 μmol, 44.0% yield) according to the procedures described herein and the synthetic scheme, using [2-chloro-4-[3-[1-[3,6-dimethyl-4-oxo-2-(1-piperidyl)chromen-8-yl]ethylamino]-2-pyridyl]-6-formyl-phenyl]trifluoromethanesulfonate (1.00 g, 1.51 mmol, 1 eq) and B₂Pin₂ (1.91 g, 7.53 mmol, 5 eq). ¹H NMR (MeOD, 400 MHz) δ 7.85 (d, J=4.8 Hz, 1H), 7.76 (s, 1H), 7.45-7.42 (s, 3H), 7.12-7.10 (m, 1H), 6.97-6.96 (m, 1H), 5.80 (d, J=16.4 Hz, 1H), 5.10-5.04 (m, 1H), 3.42 (s, 4H), 2.38 (d, J=8.4 Hz, 3H), 2.03-2.02 (m, 3H), 1.59 (s, 6H), 1.57 (t, J=6.8 Hz, 3H).

### Preparation of 8-[1-[[2-(8-chloro-1-hydroxy-2,3,1-benzoxazaborinin-6-yl)-3-pyridyl]am ino]ethyl]-3,6-dimethyl-2-(1-piperidyl)chromen-4-one

The title compound was prepared as an off-white solid (350 mg, 80% yield) according to the procedures described herein and the synthetic scheme, using 3-chloro-5-[3-[1-[3,6-dimethyl-4-oxo-2-(1-piperidyl)chromen-8-yl]ethylamino]-2-pyridyl]-2-(4,4,5,5-tetramethyl-1,3,2-dioxaborolan-2-yl)benzaldehyde (500 mg, 623 μmol, 1 eq) and hydroxylamine hydrochloride (105 mg, 1.25 mmol, 2 eq).

### Preparation of 8-[(1S)-1-[[2-(8-chloro-1-hydroxy-2,3,1-benzoxazaborinin-6-yl)-3-pyridyl]amino]ethyl]-3,6-dimethyl-2-(1-piperidyl)chromen-4-one and 8-[(1R)-1-[[2-(8-chloro-1-hydroxy-2,3,1-benzoxazaborinin-6-yl)-3-pyridyl]amino]ethyl]-3,6-dimethyl-2-(1-piperidyl)chromen-4-one

8-[1-[[2-(8-chloro-1-hydroxy-2,3,1-benzoxazaborinin-6-yl)-3-pyridyl]amino]ethyl]-3,6-dimethyl-2-(1-piperidyl)chromen-4-one (350 mg, 628 μmol, 1 eq) was separated by SFC (column: ChiralPak IH, 250×30 mm×10 um; mobile phase: [CO₂-EtOH]; B %: 45%, isocratic elution mode). Then the two obtained enantiomers were purified by Prep-HPLC (column: Waters Xbridge BEH C18 100×30 mm×10 um; mobile phase: [H2O (10 mM □H4HCO3)-AC□]; gradient:20%-50% B over 8.0 min) and then carried out further 2^{nd} purification by Prep-HPLC (column: Waters Xbridge Prep OBD C18 150×40 mm×10 um; mobile phase: [H₂O (10 mM NH4HCO3)-AC□]; gradient:10%-40% B over 8.0 min) to give the title compound, Isomer 1 (2.8 mg, 5.03 gmol) and the title compound, Isomer 2 (8.3 mg, 14.91 Ξmol) as a white solid. Isomer 1: ¹H NMR (DMSO-d₆, 400 MHz) δ 8.66 (s, 1H), 8.05-8.04 (m, 1H), 8.03-8.02 (m, 1H), 7.96 (d, J=4.4 Hz, 1H), 7.61 (s, 1H), 7.50 (s, 1H), 7.12 (t, J=4.8 Hz, 1H), 6.94 (d, J=8.4 Hz, 1H), 5.81 (d, J=6.8 Hz, 1H), 4.94 (t, J=6.8 Hz, 1H), 3.26 (s, 4H), 2.32 (s, 3H), 1.89 (s, 3H), 1.58 (s, 6H), 1.51 (d, J=6.8 Hz, 3H). MS (ESI): mass calcd. For C₃₀H₃₀BClN₄O₄ 556.20, m/z found 557.1 [M+H]⁺. HPLC: 99.47 (220 nm), 99.80 (254 nm). Chiral purity: 100% ee. Isomer 2: ¹H NMR (DMSO-d₆, 400 MHz) δ 8.66 (s, 1H), 8.05-8.04 (m, 1H), 8.03-8.02 (m, 1H), 7.96 (d, J=4.4 Hz, 1H), 7.61 (s, 1H), 7.50 (s, 1H), 7.12 (t, J=4.8 Hz, 1H), 6.94 (d, J=8.4 Hz, 1H), 5.81 (d, J=6.8 Hz, 1H), 4.94 (t, J=6.8 Hz, 1H), 3.26 (s, 4H), 2.32 (s, 3H), 1.89 (s, 3H), 1.58 (s, 6H), 1.51 (d, J=6.8 Hz, 3H). MS (ESI): mass calcd. For C₃₀H₃₀BClN₄O₄ 556.20, m/z found 557.2 [M+H]⁺. HPLC: 98.07 (220 nm), 98.99 (254 nm). Chiral purity: 95.38% ee.

### Example 83

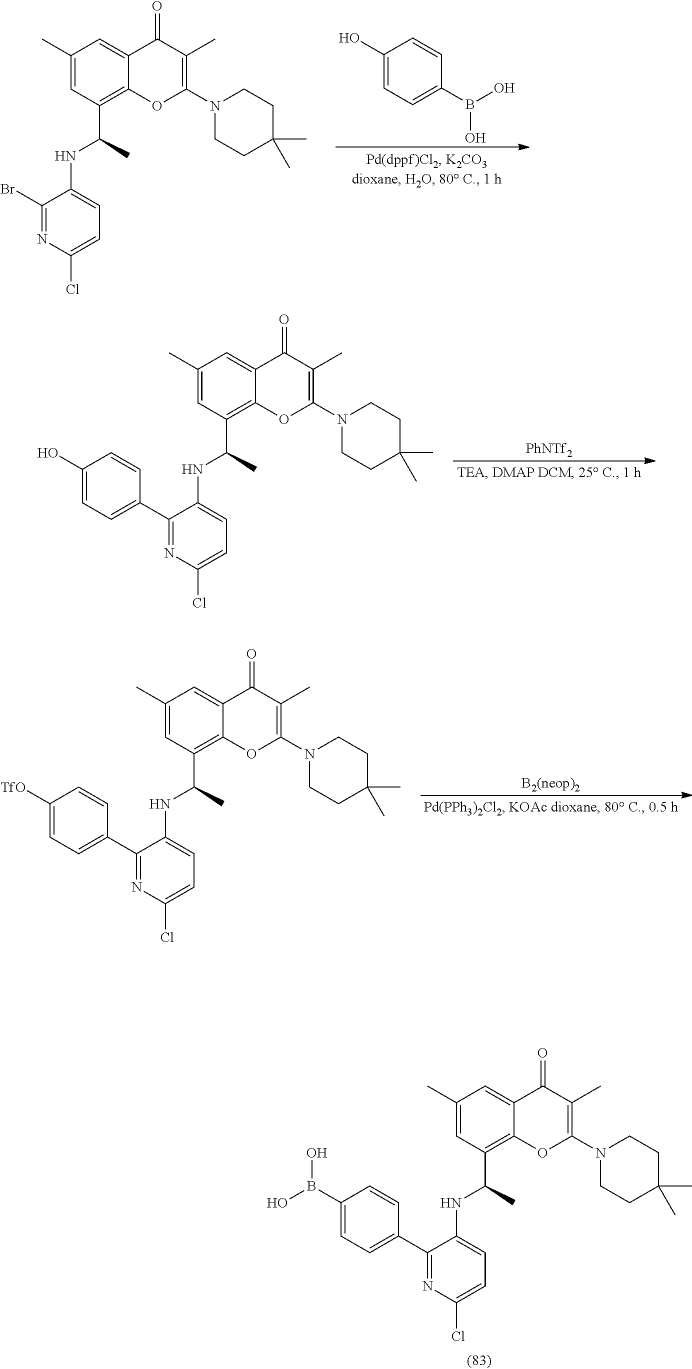

### Preparation of 8-[(1R)-1-[[6-chloro-2-(4-hydroxyphenyl)-3-pyridyl]amino]ethyl]-2-(4,4-dimethyl-1-piperidyl)-3,6-dimethyl-chromen-4-one

To a mixture of 8-[(1R)-1-[(2-bromo-6-chloro-3-pyridyl)amino]ethyl]-2-(4,4-dimethyl-1-piperidyl)-3,6-dimethyl-chromen-4-one (500 mg, 963 μmol, 1 eq) and (4-hydroxyphenyl) boronic acid (199 mg, 1.45 mmol, 1.5 eq) in dioxane (5 mL)/H₂O (1 mL) were added K₂CO₃ (359 mg, 2.60 mmol, 2.7 eq) and Pd(dppf)Cl₂ (70.5 mg, 96.3 μmol, 0.1 eq) in turns at 20° C., the reaction was degassed and purged with N₂ for 3 times, and then stirred at 80° C. for 1 h under N₂ atmosphere. The reaction mixture was cooled to 20° C., filtered and the filtrate was concentrated under reduced pressure. The residue was purified by flash silica gel chromatography (BIOTAGE®; 20 g SepaFlash® Silica Flash Column, Eluent of 0~70% Ethyl acetate/Petroleum ether gradient @60 m/min) to give the title compound (500 mg, 939 mol, 97.5% yield) as a yellow solid. ¹H NMR (DMSO-d₆, 400 MHz) δ 9.76 (s, 1H), 7.62 (d, J=1.2 Hz, 1H), 7.53 (d, J=8.8 Hz, 2H), 7.44 (d, J=2.0 Hz, 1H), 7.05 (d, J=8.4 Hz, 1H), 6.93-6.86 (m, 2H), 6.83 (d, J=8.4 Hz, 1H), 5.30 (d, J=6.8 Hz, 1H), 4.96-4.89 (m, 1H), 3.32-3.28 (m, 4H), 2.31 (s, 3H), 1.90 (s, 3H), 1.51 (d, J=6.8 Hz, 3H), 1.41 (t, J=5.6 Hz, 4H), 0.97 (s, 6H)

### Preparation of [4-[6-chloro-3-[[(1R)-1-[2-(4,4-dimethyl-1-piperidyl)-3,6-dimethyl-4-oxo-chromen-8-yl]ethyl]amino]-2-pyridyl]phenyl]trifluoromethanesulfonate

To a solution of 8-[(1R)-1-[[6-chloro-2-(4-hydroxyphenyl)-3-pyridyl]amino]ethyl]-2-(4,4-dimethyl-1-piperidyl)-3,6-dimethyl-chromen-4-one (450 mg, 845 μmol, 1 eq), TEA (171 mg, 1.69 mmol, 235 μL, 2 eq) and DMAP (10.3 mg, 84.5 μmol, 0.1 eq) in DCM (5 mL) was added 1,1,1-trifluoro-N-phenyl-N-(trifluoromethylsulfonyl)methanesulfonamide (392 mg, 1.10 mmol, 1.3 eq) portion-wise at 25° C., the mixture was stirred at 25° C. for 1 h. The reaction mixture was partitioned between H₂O (20 mL)/DCM (50 mL). The organic phase was separated, washed with brine, dried over Na₂SO₄, filtered and concentrated under reduced pressure. The residue was purified by flash silica gel chromatography (BIOTAGE®; 20 g SepaFlash® Silica Flash Column, Eluent of 0~70% Ethyl acetate/Petroleum ether gradient @60 mL/min) to give the title compound (550 mg, 828 μmol, 97.9% yield) as yellow oil. ¹H NMR (DMSO-d₆, 400 MHz) δ 7.94 (d, J=8.8 Hz, 2H), 7.63 (s, 2H), 7.61 (s, 1H), 7.45 (d, J=1.6 Hz, 1H), 7.15 (d, J=8.4 Hz, 1H), 6.88 (d, J=8.8 Hz, 1H), 5.84 (d, J=6.4 Hz, 1H), 4.96-4.92 (m, 1H), 3.41-3.34 (m, 4H), 2.31 (s, 3H), 1.91 (s, 3H), 1.52 (d, J=6.4 Hz, 3H), 1.44 (t, J=5.6 Hz, 4H), 0.98 (s, 6H).

### Preparation of [4-[6-chloro-3-[[(1R)-1-[2-(4,4-dimethyl-1-piperidyl)-3,6-dimethyl-4-oxo-chromen-8-yl]ethyl]amino]-2-pyridyl]phenyl]boronic acid

To a mixture of [4-[6-chloro-3-[[(1R)-1-[2-(4,4-dimethyl-1-piperidyl)-3,6-dimethyl-4-oxo-chromen-8-yl]ethyl]amino]-2-pyridyl]phenyl]trifluoromethanesulfonate (500 mg, 752 μmol, 1 eq) and 2-(5,5-dimethyl-1,3,2-dioxaborinan-2-yl)-5,5-dimethyl-1,3,2-dioxaborinane (680 mg, 3.01 mmol, 4 eq) in dioxane (7 mL) were added KOAc (147 mg, 1.51 mmol, 2 eq) and Pd(PPh₃)₂Cl₂ (53 mg, 75.2 gmol, 0.1 eq) in turns at 20° C., the reaction mixture was degassed and purged with N₂ for 3 times, and stirred at 80° C. for 0.5 h under N₂ atmosphere. The reaction mixture was cooled to 20° C., filtered and directly purified by prep-HPLC (column: Waters Xbridge BEH C18 250×50 mm×10 um; mobile phase: [H2O (10 mM NH4HCO3)-ACN]; gradient: 40%-80% B over 10.0 min) to give the title compound (139 mg, 248 μmol, 33.0% yield) as an off-white solid. ¹H NMR (DMSO-d₆, 400 MHz) δ 8.13 (s, 2H), 7.92 (d, J=8.0 Hz, 2H), 7.65 (d, J=8.0 Hz, 2H), 7.63 (s, 1H), 7.45 (d, J=2.0 Hz, 1H), 7.13 (d, J=8.8 Hz, 1H), 6.91 (d, J=8.8 Hz, 1H), 5.41 (d, J=6.8 Hz, 1H), 4.98-4.92 (m, 1H), 3.31-3.25 (m, 4H), 2.32 (s, 3H), 1.90 (s, 3H), 1.52 (d, J=6.4 Hz, 3H), 1.40 (t, J=5.6 Hz, 4H), 0.96 (s, 6H) MS (ESI): mass calcd. For C₃₁H₃₅BClN₃O₄ 559.24, m/z found 560.2 [M+H]⁺. HPLC: 97.92% (220 nm), 98.48% (254 nm). Chiral purity: 98.16 ee %.

### Example 84

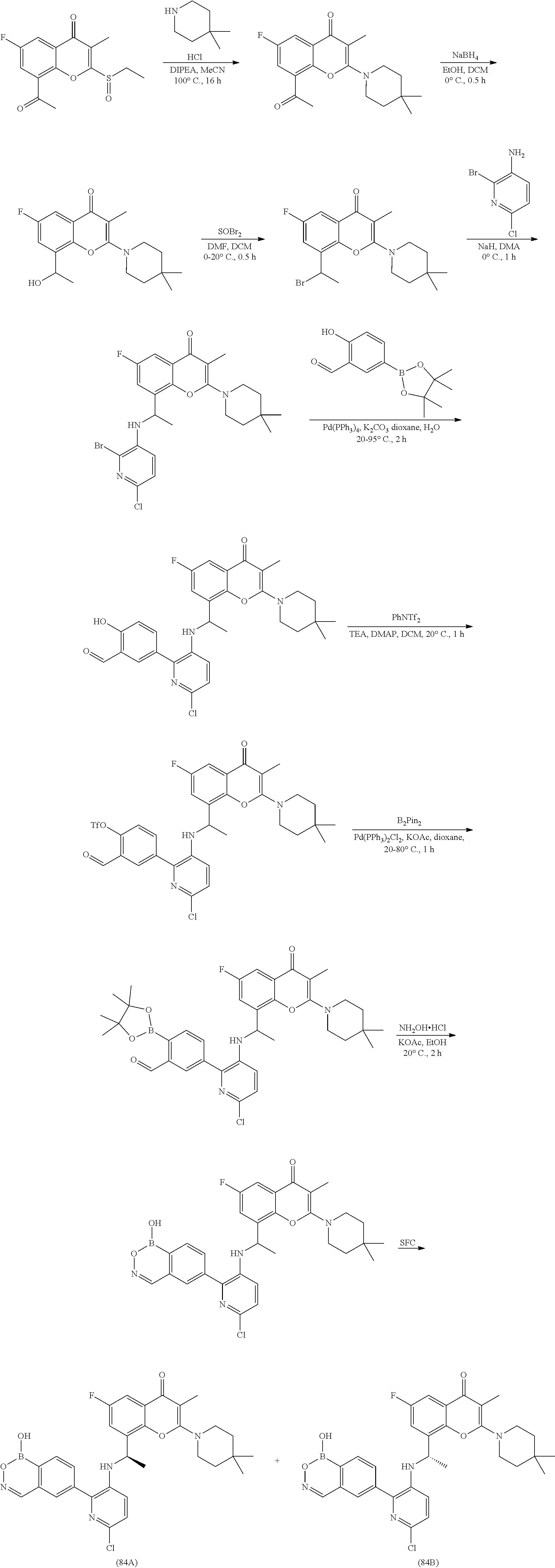

### Preparation of 8-acetyl-2-(4,4-dimethylpiperidin-1-yl)-6-fluoro-3-methyl-4H-chromen-4-one

A mixture of 8-acetyl-2-(ethylsulfinyl)-6-fluoro-3-methyl-4H-chromen-4-one (3.50 g, 11.8 mmol, 1 eq), 4,4-dimethylpiperidine (2.01 g, 17.7 mmol, 1.5 eq) and DIEA (4.58 g, 35.44 mmol, 6.17 mL, 3 eq) in MeCN (40 mL) was degassed and purged with N₂ for 3 times at 20° C., and then the reaction mixture was heated to 100° C. stirred at 100° C. for 16 h under N₂ atmosphere. The reaction mixture was cooled to 0° C. and then directly filtered. The filter cake was purified by flash silica gel chromatography (Biotage®; 80 g SepaFlash® Silica Flash Column, Eluent of 30~50% Ethyl acetate/Petroleum ether gradient @120 mL/min) to give the title compound (2.80 g, 8.45 mmol, 71.53% yield) as a yellow solid. ¹H NMR (CDCl3, 400 MHz) δ 8.03 (dd, J=7.6 Hz, 3.2 Hz, 1H), 7.80 (dd, J=8.0 Hz, 3.2 Hz, 1H), 3.46 (t, J=5.6 Hz, 4H), 2.73 (s, 3H), 2.05 (s, 3H), 1.53 (t, J=5.6 Hz, 4H), 1.05 (s, 6H).

### Preparation of 2-(4,4-dimethylpiperidin-1-yl)-6-fluoro-8-(1-hydroxyethyl)-3-methyl-4H-chromen-4-one

To a solution of 8-acetyl-2-(4,4-dimethylpiperidin-1-yl)-6-fluoro-3-methyl-4H-chromen-4-one (2.80 g, 8.45 mmol, 1 eq) in EtOH (20 mL)/DCM (20 mL) was added NaBH₄ (639 mg, 16.9 mmol, 2 eq) in portions at 0° C. The reaction mixture was stirred at 0° C. for 0.5 h. The reaction mixture was poured into sat.aq. NH₄Cl (100 mL) at 0° C. and then extracted with DCM (100 mL×2). The combined organic layers were washed dried over Na₂SO₄ (20 g), filtered and concentrated under reduced pressure to give the title compound (2.80 g, 8.40 mmol, 99.40% yield) as a yellow solid. ¹H NMR (CDCl3, 400 MHz) δ 7.54 (dd, J=8.4 Hz, 3.2 Hz, 1H), 7.36 (dd, J=8.8 Hz, 3.2 Hz, 1H), 5.33 (q, J=6.4 Hz, 1H), 3.44-3.41 (m, 4H), 2.03 (s, 3H), 1.55-1.52 (m, 7H), 1.06 (s, 6H).

### Preparation of 8-(1-bromoethyl)-2-(4,4-dimethylpiperidin-1-yl)-6-fluoro-3-methyl-4H-chromen-4-one

To a solution of 2-(4,4-dimethylpiperidin-1-yl)-6-fluoro-8-(1-hydroxyethyl)-3-methyl-4H-chromen-4-one (2.70 g, 8.10 mmol, 1 eq) in DCM (30 mL)/DMF (59.1 mg, 809 μmol, 62.3 μL, 0.1 eq) was added SOBr₂ (2.53 g, 12.1 mmol, 941 μL, 1.5 eq) dropwise at 0° C., the reaction mixture was warmed to 20° C. and stirred at 20° C. for 0.5 h. The reaction mixture was quenched by H₂O (20 mL) at 0° C., and then extracted with DCM (30 mL×3). The combined organic layers were washed with brine (30 mL), dried over anhydrous Na₂SO₄, filtered and concentrated under reduced pressure to give the title compound (2.8 g, 7.07 mmol, 87.24% yield) as a yellow solid, which was used directly for next step without further purification.

### Preparation of 8-(1-((2-bromo-6-chloropyridin-3-yl)amino)ethyl)-2-(4,4-dimethyl piperidin-1-yl)-6-fluoro-3-methyl-4H-chromen-4-one

To a solution of 2-bromo-6-chloropyridin-3-amine (785 mg, 3.79 mmol, 1 eq) in DMA (20 mL) was added NaH (196 mg, 4.92 mmol, 1.3 eq) in portions at 0° C., the mixture was stirred at this temperature for 30 min. Then a solution of 8-(1-bromoethyl)-2-(4,4-dimethylpiperidin-1-yl)-6-fluoro-3-methyl-4H-chromen-4-one (2.0 g, 3.79 mmol, 1 eq) in DMA (20 mL) was added dropwise to the above reaction mixture at 0° C. and the resulting mixture was stirred at 0° C. for 0.5 h. The reaction mixture was quenched by adding water (60 mL) at 0° C., and then filtered to give the title compound (1.4 g, 2.68 mmol, 70.74% yield) as a yellow solid. ¹H NMR (CDCl₃, 400 MHz) δ 7.75 (dd, J=8.0 Hz 3.2 Hz, 1H), 7.22 (dd, J=8.4 Hz 3.2 Hz, 1H), 6.98 (d, J=8.4 Hz, 1H), 6.43 (d, J=8.4 Hz, 1H), 4.94-4.89 (m, 1H), 4.81-4.80 (m, 1H), 3.44-3.41 (m, 4H), 2.06 (s, 3H), 1.72 (d, J=6.8 Hz, 3H), 1.55-1.52 (m, 4H), 1.05 (s, 6H).

### Preparation of 5-(6-chloro-3-((1-(2-(4,4-dimethylpiperidin-1-yl)-6-fluoro-3-methyl-4-oxo-4H-chromen-8-yl)ethyl)amino)pyridin-2-yl)-2-hydroxybenzaldehyde

A mixture of 8-(1-((2-bromo-6-chloropyridin-3-yl)amino)ethyl)-2-(4,4-dimethylpiperidin-1-yl)-6-fluoro-3-methyl-4H-chromen-4-one (200 mg, 382 μmol, 1 eq), 2-hydroxy-5-(4,4,5,5-tetramethyl-1,3,2-dioxaborolan-2-yl)benzaldehyde (113 mg, 459 μmol, 1.2 eq), Pd(PPh₃)₄ (44.2 mg, 38.2 gmol, 0.1 eq) and K₂CO₃ (105 mg, 765 μmol, 2 eq) in dioxane (2 mL)/H₂O (0.2 mL) was degassed and purged with N₂ for 3 times at 20° C., and then the mixture was heated to 95° C. and stirred at 95° C. for 2 h under N₂ atmosphere. The reaction mixture was filtered and the filtrate was concentrated under reduced pressure to give a residue. The residue was purified by flash silica gel chromatography (Biotage®; 12 g SepaFlash® Silica Flash Column, Eluent of 30-40% Ethyl acetate/Petroleum ether gradient @50 m/min) to give the title compound (200 mg, 354.58 μmol, 92.69% yield) as a yellow solid. ¹H NMR (CDCl₃, 400 MHz) δ 10.30 (s, 1H), 8.02 (d, J=2.0 Hz, 1H), 7.88-7.86 (m, 1H), 7.48-7.45 (m, 2H), 7.09-7.05 (m, 2H), 6.88-6.85 (m, 1H), 5.75-5.72 (m, 1H), 4.98-4.87 (m, 1H), 3.42-3.41 (m, 4H), 1.92 (s, 3H), 1.52 (d, J=6.4 Hz, 3H), 1.44-1.42 (m, 4H), 0.98 (s, 6H).

### Preparation of 4-(6-chloro-3-((1-(2-(4,4-dimethylpiperidin-1-yl)-6-fluoro-3-methyl-4-oxo-4H-chromen-8-yl)ethyl)amino)pyridin-2-yl)-2-formylphenyltrifluoromethanesulfonate

A mixture of 5-(6-chloro-3-((1-(2-(4,4-dimethylpiperidin-1-yl)-6-fluoro-3-methyl-4-oxo-4H-chromen-8-yl)ethyl)amino)pyridin-2-yl)-2-hydroxybenzaldehyde (800 mg, 1.42 mmol, 1 eq), PhNTf₂ (1.01 g, 2.84 mmol, 2 eq), TEA (287 mg, 2.84 mmol, 394 μL, 2 eq) and DMAP (17.3 mg, 141 μmol, 0.1 eq) in DCM (15 mL) was degassed and purged with N₂ for 3 times at 20° C., and then the reaction mixture was stirred at 20° C. for 1 h under N₂ atmosphere. The reaction mixture was concentrated under reduced pressure to give a residue. The residue was purified by flash silica gel chromatography (Biotage® 12 g SepaFlash® Silica Flash Column, Eluent of 35-40% Ethyl acetate/Petroleum ether gradient @75 mL/min) to give the title compound (800 mg, 1.15 mmol, 81.03% yield) as yellow solid. ¹H NMR (CDCl₃, 400 MHz) δ 10.35 (s, 1H), 8.42 (d, J=2.0 Hz, 1H), 8.13 (dd, J=8.4 Hz 2.4 Hz, 1H), 7.79-7.79 (m, 1H), 7.60 (d, J=8.8 Hz, 1H), 7.24-7.22 (m, 1H), 7.07-7.05 (m, 1H), 6.64-6.62 (m, 1H), 4.96-4.91 (m, 1H), 4.40-4.31 (m, 1H), 3.45-3.42 (m, 4H), 2.09 (s, 3H), 1.64-1.60 (m, 3H), 1.55-1.52 (m, 4H), 1.05 (s, 6H).

### Preparation of 5-(6-chloro-3-((1-(2-(4,4-dimethylpiperidin-1-yl)-6-fluoro-3-methyl-4-oxo-4H-chromen-8-yl)ethyl)amino)pyridin-2-yl)-2-(4,4,5,5-tetramethyl-1,3,2-dioxaborolan-2-yl)benzaldehyde

A mixture of 4-(6-chloro-3-((1-(2-(4,4-dimethylpiperidin-1-yl)-6-fluoro-3-methyl-4-oxo-4H-chromen-8-yl)ethyl)amino)pyridin-2-yl)-2-formylphenyltrifluoromethanesulfonate (250 mg, 359 mol, 1 eq), B₂Pin₂ (456 mg, 1.80 mmol, 5 eq), KOAc (70 mg, 718 μmol, 2 eq) and Pd(PPh₃)₂Cl₂ (25. mg, 35.9 μmol, 0.1 eq) in dioxane (3 mL) was degassed and purged with N₂ for 3 times at 20° C., and then the mixture was heated to 80° C. and stirred at 80° C. for 1 h under N₂ atmosphere. The reaction mixture was filtered and the filtrate was concentrated under reduced pressure to give the title compound (250 mg, crude) as a yellow solid, which was used directly for next step without further purification.

### Preparation of 8-(1-((6-chloro-2-(1-hydroxy-1H-benzo[d][1,2,6]oxazaborinin-6-yl) pyridin-3-yl)amino)ethyl)-2-(4,4-dimethylpiperidin-1-yl)-6-fluoro-3-methyl-4H-chromen-4-one

A mixture of 5-(6-chloro-3-((1-(2-(4,4-dimethylpiperidin-1-yl)-6-fluoro-3-methyl-4-oxo-4H-chromen-8-yl)ethyl)amino)pyridin-2-yl)-2-(4,4,5,5-tetramethyl-1,3,2-dioxaborolan-2-yl)benzaldehyde (250 mg, 370 μmol, 1 eq), KOAc (72 mg, 741 μmol, 2 eq) and NH₂OH·HCl (51 mg, 741 mol, 2 eq) in EtOH (3 ml) was degassed and purged with N₂ for 3 times at 20° C., and then the reaction mixture was stirred at 20° C. for 2 h under N₂ atmosphere. The reaction mixture was quenched by H₂O (30 mL) at 20° C. and then the resulting suspension was directly filtered. The filter cake was purified by prep-HPLC (column: 2_Phenomenex Gemini C18 75×40 mm×3 um; mobile phase: [1H₂₀ (10 mM NH₄HCO₃)-ACD]; gradient: 15%-55% B over 8.0 min) to give the title compound (68 mg, 115 μmol, 31.13% yield) as a white solid.

### Preparation of (S)-8-(1-((6-chloro-2-(1-hydroxy-JH-benzo[d][1,2,6]oxazaborinin-6-yl) pyridin-3-yl)amino)ethyl)-2-(4,4-dimethylpiperidin-1-yl)-6-fluoro-3-methyl-4H-chromen-4-one & (R)-8-(1-((6-chloro-2-(1-hydroxy-JH-benzo[d][1,2,6]oxazaborinin-6-yl)pyridin-3-yl) amino)ethyl)-2-(4,4-dimethylpiperidin-1-yl)-6-fluoro-3-methyl-4H-chromen-4-one

8-(1-((6-chloro-2-(1-hydroxy-1H-benzo[d]1[1,2,6]oxazaborinin-6-yl)pyridin-3-yl)amino)ethyl)-2-(4,4-dimethylpiperidin-1-yl)-6-fluoro-3-methyl-4H-chromen-4-one (68 mg, 115 μmol, 1 eq) was purified by SFC (column: ChiralPak IH, 250×30 mm×10 um; mobile phase: [CO2-IPA]; B %: 50%, isocratic elution mode) to give the title compound, Isomer 1 (27.0 mg, 45.53 μmol, 39.43% yield) as white solid and the title compound, Isomer 2 (27.2 mg, 45.54 μmol, 39.44% yield) as a white solid.

Isomer 1: ¹H NMR (DMSO-d₆, 400 MHz) δ 9.47 (s, 1H), 8.72 (s, 1H), 8.17-8.15 (m, 1H), 8.07-8.06 (m, 2H), 7.49-7.46 (m, 2H), 7.19 (d, J=8.8 Hz, 1H), 6.97 (d, J=8.8 Hz, 1H), 5.94-5.92 (m, 1H), 4.96-4.89 (m, 1H), 3.35-3.33 (m, 4H), 1.91 (s, 3H), 1.52 (d, J=6.4 Hz, 3H), 1.43-1.40 (m, 4H), 0.97 (s, 6H). MS (ESI): mass calcd. For C₃₁H₃₁BClFN₄O₄ 588.87, m/z found 589.1 [M+H]⁺. HPLC: 99.30% (220 nm), 99.75% (254 nm). Chiral purity: 100% ee. Isomer 2: ¹H NMR (DMSO-d₆, 400 MHz) δ 9.46 (s, 1H), 8.71 (s, 1H), 8.16-8.14 (m, 1H), 8.07-8.06 (m, 2H), 7.48-7.46 (m, 2H), 7.18 (d, J=8.4 Hz, 1H), 6.97 (d, J=8.8 Hz, 1H), 5.94-5.92 (m, 1H), 4.96-4.89 (m, 1H), 3.32-3.32 (m, 4H), 1.91 (s, 3H), 1.52 (d, J=6.4 Hz, 3H), 1.43-1.40 (m, 4H), 0.97 (s, 6H). MS (ESI): mass calcd. For C₃₁H₃₁BClFN₄O₄ 588.87, m/z found 589.1 [M+H]⁺. HPLC: 98.60% (220 nm), 99.28% (254 nm). Chiral purity: 97.66% ee

### Example 85

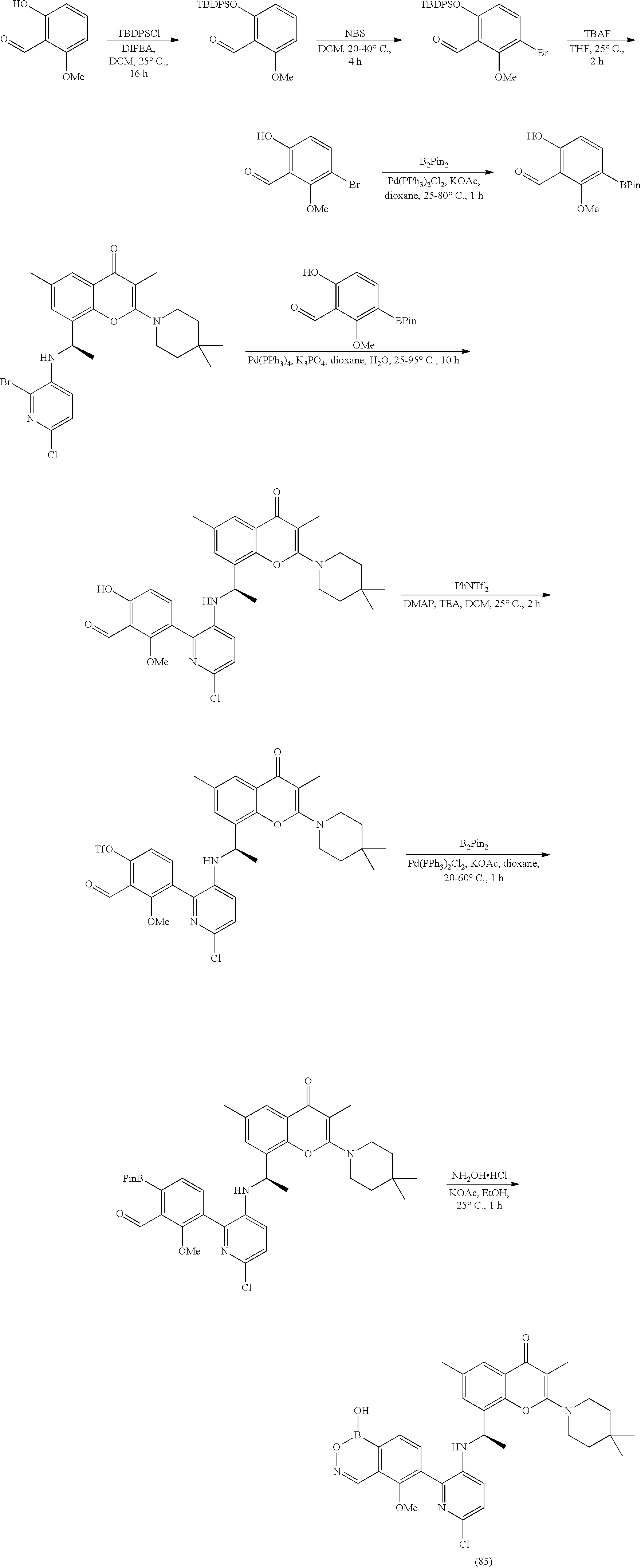

### Preparation of 2-[tert-butyl(diphenyl)silyl]oxy-6-methoxy-benzaldehyde

To a solution of 2-hydroxy-6-methoxy-benzaldehyde (10.00 g, 65.73 mmol, 1 eq) in DCM (150 mL) was added DIPEA (25.48 g, 197.18 mmol, 34.34 mL, 3 eq) and tert-butyl-chloro-diphenyl-silane (54.20 g, 197.18 mmol, 50.46 mL, 3 eq) in turns at 25° C., the reaction mixture was stirred at 25° C. for 16 h. The reaction mixture was concentrated under reduced pressure and the residue was purified by flash silica gel chromatography (Biotage®; 330 g SepaFlash® Silica Flash Column, Eluent of 0-30% Ethyl acetate/Petroleum ether gradient @120 mL/min) to give the title compound (20.00 g, 51.21 mmol, 77.92% yield) as a white solid. ¹H NMR (DMSO-d₆, 400 MHz) δ 10.57 (s, 1H), 7.69 (d, J=8.0 Hz, 4H), 7.49-7.46 (m, 6H), 7.16 (t, J=8.0 Hz, 1H), 6.66 (d, J=8.4 Hz, 1H), 5.98 (d, J=8.0 Hz, 1H), 3.82 (s, 3H), 1.03 (s, 9H).

### Preparation of 3-bromo-6-[tert-butyl(diphenyl)silyl]oxy-2-methoxy-benzaldehyde

To a solution of 2-[tert-butyl(diphenyl)silyl]oxy-6-methoxy-benzaldehyde (15.00 g, 38.41 mmol, 1 eq) in DCM (150 mL) was added NBS (7.52 g, 42.25 mmol, 1.1 eq) in portions at 20° C., the reaction mixture was heated to 40° C. and stirred at 40° C. for 4 h. The reaction mixture was cooled to 20° C. and concentrated under reduced pressure to give a residue. The residue was purified by flash silica gel chromatography (Biotage®; 80 g SepaFlash® Silica Flash Column, Eluent of 0~20% Ethyl acetate/Petroleum ether gradient @80 mL/min) to give the title compound (5.00 g, 10.65 mmol, 27.73% yield) as a yellow solid. ¹H NMR (DMSO-d₆, 400 MHz) δ 10.49 (s, 1H), 7.69 (d, J=8.0 Hz, 4H), 7.51-7.46 (m, 6H), 7.39-7.38 (m, 1H), 6.15 (d, J=9.2 Hz, 1H), 3.83 (s, 3H), 1.03 (s, 9H).

### Preparation of 3-bromo-6-hydroxy-2-methoxy-benzaldehyde

To a solution of 3-bromo-6-[tert-butyl(diphenyl)silyl]oxy-2-methoxy-benzaldehyde (5.00 g, 10.65 mmol, 1 eq) in THF (50 mL) was added TBAF (1 M, 12.78 mL, 1.2 eq) dropwise at 25° C., the reaction mixture was stirred at 25° C. for 2 h. The reaction mixture was quenched with H₂O (40 mL) and then extracted with EtOAc (50 mL×3). The combined organic layers were washed with brine (30 mL×2), dried over Na₂SO₄, filtered and concentrated under reduced pressure to give a residue. The residue was purified by flash silica gel chromatography (Biotage®; 660 g SepaFlash® Silica Flash Column, Eluent of 0~10% Ethyl acetate/Petroleum ether gradient @120 mL/min) to give the title compound (2.00 g, 8.63 mmol, 81.03% yield) as a yellow solid. ¹H NMR (DMSO-d₆, 400 MHz) δ 11.37 (s, 1H), 10.22 (s, 1H), 7.77 (d, J=8.8 Hz, 1H), 6.76 (d, J=9.2 Hz, 1H), 3.88 (s, 3H).

### Preparation of 6-hydroxy-2-methoxy-3-(4,4,5,5-tetramethyl-1,3,2-dioxaborolan-2-yl)benzaldehyde

A mixture of 3-bromo-6-hydroxy-2-methoxy-benzaldehyde (4.00 g, 17.31 mmol, 1 eq), B₂Pin₂ (21.98 g, 86.56 mmol, 5 eq), KOAc (3.40 g, 34.63 mmol, 2 eq) and Pd(PPh₃)₂Cl₂ (1.22 g, 1.73 mmol, 0.1 eq) in dioxane (40 mL) at 25° C. was degassed and purged with N₂ for 3 times, and then the reaction mixture was stirred at 80° C. for 1 h under N₂ atmosphere. The reaction mixture was cooled to 25° C., quenched with H₂O (50 mL) and extracted with EtOAc (50 mL×3). The combined organic layers were washed with brine (20 mL×2), dried over Na₂SO₄, filtered and concentrated under reduced pressure to give a residue. The residue was purified by prep-HPLC (column: Welch Xtimate C18 250×100 mm×10 um; mobile phase: [H₂O (10 mM NH₄HCO₃)-AC□]; gradient: 45%-80% B over 18.0 min) to give the title compound (1.36 g, 4.89 mmol, 28.25% yield) as a yellow solid. ¹H NMR (DMSO-d₆, 400 MHz) δ 11.79 (s, 1H), 10.24 (s, 1H), 7.77 (d, J=8.4 Hz, 1H), 6.71 (d, J=8.8 Hz, 1H), 3.90 (s, 3H), 1.30 (s, 12H).

### Preparation of (R)-3-(6-chloro-3-((1-(2-(4,4-dimethylpiperidin-1-yl)-3,6-dimethyl-4-oxo-4H-chromen-8-yl)ethyl)amino)pyridin-2-yl)-6-hydroxy-2-methoxybenzaldehyde

A mixture of 8-[(1R)-1-[(2-bromo-6-chloro-3-pyridyl)amino]ethyl]-2-(4,4-dimethyl-1-piperidyl)-3,6-dimethyl-chromen-4-one (600 mg, 1.16 mmol, 1 eq), 6-hydroxy-2-methoxy-3-(4,4,5,5-tetramethyl-1,3,2-dioxaborolan-2-yl)benzaldehyde (643 mg, 2.31 mmol, 2 eq), K₃PO₄ (491 mg, 2.31 mmol, 2 eq) and Pd(PPh₃)₄ (267 mg, 231.27 gmol, 0.2 eq) in dioxane (15 mL)/H₂O (5 mL) was degassed and purged with N₂ for 3 times at 25° C., and then the reaction mixture was stirred at 95° C. for 10 h under N₂ atmosphere. The reaction mixture was cooled to 25° C., diluted with H₂O (5 mL) and extracted with EtOAc (5 mL×3). The combined organic layers were washed with brine, dried over Na₂SO₄, filtered and concentrated under reduced pressure to give a residue. The residue was purified by flash silica gel chromatography (Biotage®; 12 g SepaFlash® Silica Flash Column, Eluent of 0~50% Ethyl acetate/Petroleum ether gradient @40 m/min) to give the title compound (600 mg, 1.02 mmol, 87.93% yield) as yellow oil. ¹H NMR (DMSO-d₆, 400 MHz) δ 11.72 (s, 1H), 10.32 (s, 1H), 7.62 (s, 1H), 7.52 (d, J=8.8 Hz, 2H), 7.15 (d, J=8.8 Hz, 1H), 6.85 (d, J=8.8 Hz, 2H), 5.37 (d, J=6.8 Hz, 1H), 4.98 (t, J=6.8 Hz, 1H), 3.93 (s, 3H), 3.39-3.35 (m, 4H), 2.30 (s, 3H), 1.91 (s, 3H), 1.50-1.45 (m, 7H), 0.99 (s, 6H).

### Preparation of (R)-4-(6-chloro-3-((1-(2-(4,4-dimethylpiperidin-1-yl)-3,6-dimethyl-4-oxo-4H-chromen-8-yl)ethyl)amino)pyridin-2-yl)-2-formyl-3-methoxyphenyltrifluoromethanesulfonate

To a solution of 3-[6-chloro-3-[[(1R)-1-[2-(4,4-dimethyl-1-piperidyl)-3,6-dimethyl-4-oxo-chromen-8-yl]ethyl]amino]-2-pyridyl]-6-hydroxy-2-methoxy-benzaldehyde (600 mg, 1.02 mmol, 1 eq) in DCM (6 mL) was added 1,1,1-trifluoro-N-phenyl-N-(trifluoromethylsulfonyl) methanesulfonamide (726 mg, 2.03 mmol, 2 eq), DMAP (12 mg, 101 μmol, 0.1 eq) and TEA (205 mg, 2.03 mmol, 283 μL, 2 eq) in turns at 25° C., the reaction mixture was stirred at 25° C. for 2 h. The reaction mixture was concentrated under reduced pressure and the residue was purified by flash silica gel chromatography (Biotage®; 12 g SepaFlash® Silica Flash Column, Eluent of 0~50% Ethyl acetate/Petroleum ether gradient @60 m/min) to give the title compound (470 mg, 650.82 μmol, 64.01% yield) as yellow oil. ¹H NMR (DMSO-d₆, 400 MHz) δ 10.39 (s, 1H), 7.85 (d, J=8.8 Hz, 1H), 7.63 (s, 1H), 7.42 (d, J=8.4 Hz, 2H), 7.21 (d, J=8.8 Hz, 1H), 6.90-6.87 (m, 1H), 5.73-5.72 (m, 1H), 5.00 (t, J=6.8 Hz, 1H), 3.63 (s, 3H), 3.41-3.39 (m, 4H), 2.30 (s, 3H), 1.92 (s, 3H), 1.50-1.46 (m, 7H), 1.00 (s, 6H).

### Preparation of (R)-3-(6-chloro-3-((1-(2-(4,4-dimethylpiperidin-1-yl)-3,6-dimethyl-4-oxo-4H-chromen-8-yl)ethyl)amino)pyridin-2-yl)-2-methoxy-6-(4,4,5,5-tetramethyl-1,3,2-dioxaborolan-2-yl)benzaldehyde

A mixture of [4-[6-chloro-3-[[(1R)-1-[2-(4,4-dimethyl-1-piperidyl)-3,6-dimethyl-4-oxo-chromen-8-yl]ethyl]amino]-2-pyridyl]-2-formyl-3-methoxy-phenyl]trifluoromethanesulfonate (300 mg, 415 μmol, 1 eq), B₂Pin₂ (527 mg, 2.08 mmol, 5 eq), KOAc (81 mg, 830 μmol, 2 eq) and Pd(PPh₃)₂Cl₂ (29 mg, 41 μmol, 0.1 eq) in dioxane (5 mL) was degassed and purged with N₂ for 3 times at 25° C., and then the reaction mixture was stirred at 60° C. for 1 h under N₂ atmosphere. The reaction mixture was quenched by H₂O (5 mL) at 0° C. and then extracted with EtOAc (5 mL×3). The combined organic layers were washed with brine, dried over Na₂SO₄, filtered and concentrated under reduced pressure to give a residue. The residue was purified by flash silica gel chromatography (Biotage®; 12 g SepaFlash® Silica Flash Column, Eluent of 0~60% Ethyl acetate/Petroleum ether gradient @40 mL/min) to give the title compound (260 mg, crude, 89.40% yield) as a yellow solid, which was used directly for next step without further purification.

### Preparation of 8-[(1R)-1-[[6-chloro-2-(1-hydroxy-5-methoxy-2,3,1-benzoxazaborinin-6-yl)-3-pyridyl]amino]ethyl]-2-(4,4-dimethyl-1-piperidyl)-3,6-dimethyl-chromen-4-one

A mixture of 3-[6-chloro-3-[[(1R)-1-[2-(4,4-dimethyl-1-piperidyl)-3,6-dimethyl-4-oxo-chromen-8-yl]ethyl]amino]-2-pyridyl]-2-methoxy-6-(4,4,5,5-tetramethyl-1,3,2-dioxaborolan-2-yl)benzaldehyde (260 mg, 371 μmol, 1 eq). hydroxylamine; hydrochloride (52 mg, 743 μmol, 2 eq) and KOAc (73 mg, 743 μmol, 2 eq) in EtOH (3 ml) at 25° C. was degassed and purged with N₂ for 3 times, and then the reaction mixture was stirred at 25° C. for 1 h under N₂ atmosphere. The reaction mixture was diluted with H₂O (5 mL) at 25° C. and then solid was precipitated out. The resulting suspension was filtered and the filter cake was further washed with MeCN (5 ML). The filter cake was purified by prep-HPLC (column: 2_Phenomenex Gemini C18 75×40 mm×3 um; mobile phase: [H₂O (10 mM NH₄HCO₃)-AC□]; gradient: 10%-50% B over 8.0 min) to give the title compound (35 mg, 56 μmol, 15.20% yield) as a white solid. ¹H NMR (DMSO-d₆, 400 MHz) δ 8.80 (s, 1H), 7.88 (d, J=7.6 Hz, 1H), 7.66 (d, J=7.6 Hz, 1H), 7.62 (s, 1H), 7.42 (s, 1H), 7.21 (d, J=8.8 Hz, 1H), 6.93 (d, J=8.4 Hz, 1H), 5.38-5.37 (m, 1H), 4.98 (t, J=7.2 Hz, 1H), 3.57 (s, 3H), 3.39-3.37 (m, 4H), 2.31 (s, 3H), 1.91 (s, 3H), 1.46 (d, J=6.8 Hz, 3H), 1.42 (t, J=5.6 Hz, 4H), 0.97 (s, 6H). MS (ESI): mass calcd. For C₃₃H₃₆BClN₄O₅ 614.93, m/z found 615.2 [M+H]⁺. HPLC: 99.21% (220 nm), 99.59% (254 nm). Chiral purity: 98.3 ee %.

### Example 86

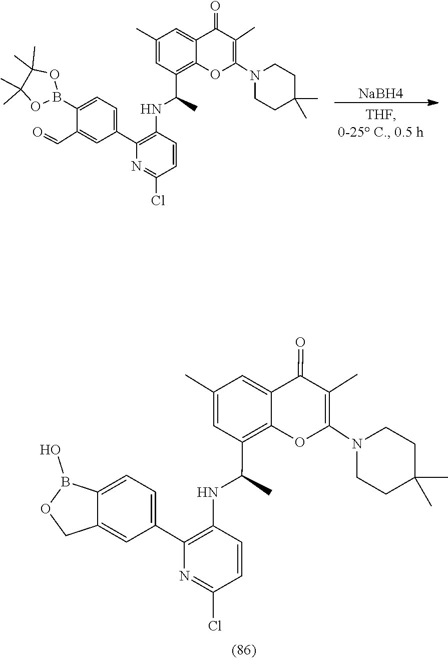

### Preparation of (R)-8-(1-((6-chloro-2-(1-hydroxy-1,3-dihydrobenzo[c][1,2]oxaborol-5-yl)pyridin-3-yl)amino)ethyl)-2-(4,4-dimethylpiperidin-1-yl)-3,6-dimethyl-4H-chromen-4-one

To a solution of 5-[6-chloro-3-[[(1R)-1-[2-(4,4-dimethyl-1-piperidyl)-3,6-dimethyl-4-oxo-chromen-8-yl]ethyl]amino]-2-pyridyl]-2-(4,4,5,5-tetramethyl-1,3,2-dioxaborolan-2-yl)benz aldehyde (300 mg, 447.73 μmol, 1 eq) in THF (3 mL) was added NaBH₄ (70 mg, 1.85 mmol, 4.13 eq) in portions at 0° C. under N₂. The reaction mixture was warmed to 25° C. and stirred at 25° C. for 0.5 h under N₂. The reaction mixture was cooled to 0° C., quenched at with H₂O (2 mL)/HCl (3M, 1 mL) and extratcted with EtOAc (2 mL×2). The combined organic layers were washed with brine (2 mL), dried over Na₂SO₄ and concentrated under reduced pressure. The residue was purified by prep-HPLC (column: Waters Xbridge BEH C18 100×30 mm×10 um; mobile phase: [H₂O (10 mM NH₄HCO₃)-ACN]; gradient: 40%-70% B over 8.0 min) to give the title compound (35.7 mg, 62.04 μmol, 13.86% yield) as a white solid. ¹H NMR (DMSO-d₆ 400 MHz) δ 9.27 (s, 1H), 7.84-7.82 (m, 1H), 7.86-7.62 (m, 3H), 7.44 (d, J=2.0 Hz, 1H), 7.15 (d, J=8.8 Hz, 1H), 6.95-6.93 (m, 1H), 5.50 (d, J=7.2 Hz, 1H), 5.02 (d, J=2.8 Hz, 2H), 4.97-4.91 (m, 1H), 3.27 (d, J=5.6 Hz, 4H), 2.32 (s, 3H), 1.90 (s, 3H), 1.50 (d, J=6.8 Hz, 3H), 1.39 (t, J=5.2 Hz, 4H), 0.96 (s, 6H). MS (ESI): mass calcd. For C₃₂H₃₅BClN₃O₄ 571.2 found 572.3 [M+H]⁺. HPLC: 99.50% (220 nm), 99.34% (254 nm). Chiral purity: 99.40% ee.

### Example 87

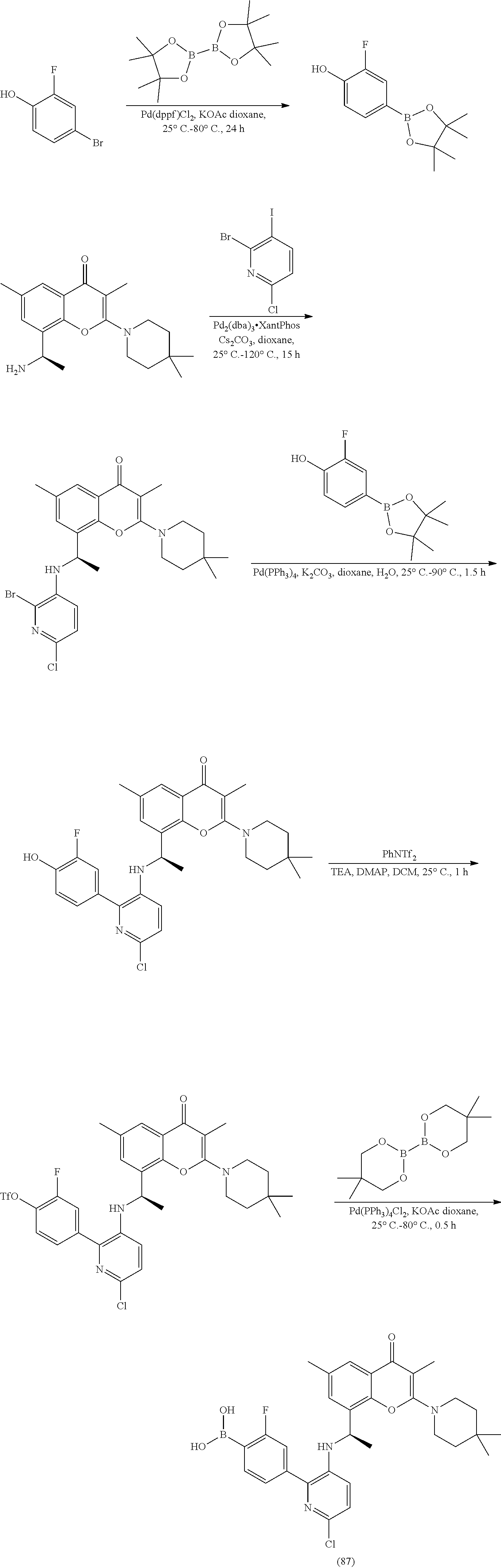

### Preparation of 2-fluoro-4-(4,4,5,5-tetramethyl-1,3,2-dioxaborolan-2-yl)phenol

To a mixture of 4-bromo-2-fluoro-phenol (5.00 g, 26.2 mmol, 1 eq) and 4,4,5,5-tetramethyl-2-(4,4,5,5-tetramethyl-1,3,2-dioxaborolan-2-yl)-1,3,2-dioxaborolane (7.64 g, 30.1 mmol, 1.15 eq) in dioxane (100 mL) were added KOAc (7.71 g, 78.5 mmol, 3 eq) and Pd(dppf)Cl₂ (957.7 mg, 1.31 mmol, 0.05 eq) at 25° C., the reaction suspension was degassed under vacuum and purged with N₂ 3 times, then the reaction mixture was directly stirred at 80° C. for 24 h under N₂. The reaction mixture was cooled to 25° C., filtered and the filtrate was concentrated under reduced pressure to give a residue. The residue was purified by flash silica gel chromatography (Biotage®; 80 g SepaFlash® Silica Flash Column, Eluent of 0~10% Ethyl acetate/Petroleum ether gradient @50 mL/min) to give the title compound (3.5.0 g, 14.70 mmol, 56.16% yield) as yellow oil. ¹H NMR (CDCl₃, 400 MHz) δ 7.55-7.45 (m, 2H), 6.99 (5,J=8.4 Hz, 1H), 5.42 (br s, 1H), 1.34 (s, 12H).

### Preparation of 8-[(1R)-1-[(2-bromo-6-chloro-3-pyridyl)amino]ethyl]-2-(4,4-dimethyl-1-piperidyl)-3,6-dimethyl-chromen-4-one

The title compound was prepared as a brown solid (2.25 g, 4.34 mmol, 94.95% yield) according to the similar procedure in Example 78, using 8-[(1R)-1-aminoethyl]-2-(4,4-dimethyl-1-piperidyl)-3,6-dimethyl-chromen-4-one (1.50 g, 4.57 mmol, 1.0 eq) and 2-bromo-6-chloro-3-iodo-pyridine (2.91 g, 9.13 mmol, 2 eq). ¹H NMR (CDCl₃, 400 MHz) δ 7.90 (s, 1H), 7.28 (d, J=2.0 Hz, 1H), 6.97 (d, J=8.4 Hz, 1H), 6.48 (d, J=8.4 Hz, 1H), 4.95-4.87 (m, 1H), 4.81 (d, J=5.2 Hz, 1H), 3.44-3.35 (m, 4H), 2.38 (s, 3H), 2.06 (s, 3H), 1.70 (d, J=6.4 Hz, 3H), 1.55-1.49 (m, 4H), 1.05 (s, 6H).

### Preparation of 8-[(1R)-1-[[6-chloro-2-(3-fluoro-4-hydroxy-phenyl)-3-pyridyl]amino]ethyl]-2-(4,4-dimethyl-1-piperidyl)-3,6-dimethyl-chromen-4-one

The title compound was prepared as a brown solid (500 mg, 908.99 μmol, 94.33% yield) according to the procedures described herein and the synthetic scheme, using 8-[(1R)-1-[(2-bromo-6-chloro-3-pyridyl)amino]ethyl]-2-(4,4-dimethyl-1-piperidyl)-3,6-dimethyl-chromen-4-one (500 mg, 963.6 mol) (1.0 eq) and 2-fluoro-4-(4,4,5,5-tetramethyl-1,3,2-dioxaborolan-2-yl)phenol (275 mg, 1.16 mmol, 1.2 eq). ¹H NMR (CDCl₃, 400 MHz) δ 7.90 (s, 1H), 7.71-7.65 (m, 1H), 7.59-7.53 (m, 1H), 7.41-7.37 (m, 1H), 7.28 (br s, 1H), 7.16 (t, J=8.4 Hz, 1H), 6.97 (d, J=8.4 Hz, 1H), 6.61 (d, J=8.4 Hz, 1H), 4.93-4.81 (m, 1H), 4.52 (br s, 1H), 3.40-3.30 (m, 4H), 2.38 (s, 3H), 2.07 (s, 3H), 1.56 (d, J=6.8 Hz, 3H), 1.53-1.49 (m, 4H), 1.04 (s, 6H).

### Preparation of [4-[6-chloro-3-[[(1R)-1-[2-(4,4-dimethyl-1-piperidyl)-3,6-dimethyl-4-oxo-chromen-8-yl]ethyl]amino]-2-pyridyl]-2-fluoro-phenyl]trifluoromethanesulfonate

The title compound was prepared as a brown solid (700 mg, 1.03 mmol, 94.08% yield) according to the procedures described herein and the synthetic scheme, using 8-[(1R)-1-[[6-chloro-2-(3-fluoro-4-hydroxy-phenyl)-3-pyridyl]amino]ethyl]-2-(4,4-dimethyl-1-piperidyl)-3,6-dimethyl-chromen-4-one (600 mg, 1.09 mmol, 1 eq) and 1,1,1-trifluoro-N-phenyl-N-(trifluoromethylsulfonyl)methanesulfonamide (390 mg, 1.09 mmol, 1 eq). ¹H NMR (CDCl₃, 400 MHz) δ 7.92 (s, 1H), 7.70 (dd, J=10.0, 2.0 Hz, 1H), 7.64 (d, J=8.8 Hz, 1H), 7.50 (t, J=8.4 Hz, 1H), 7.43-7.28 (m, 1H), 7.03 (d, J=8.8 Hz, 1H), 6.66 (d, J=8.8 Hz, 1H), 4.98-4.83 (m, 1H), 4.43 (br s, 1H), 3.42-3.33 (m, 4H), 2.38 (s, 3H), 2.07 (s, 3H), 1.60 (d, J=6.8 Hz, 3H), 1.54-1.49 (m, 4H), 1.04 (s, 6H).

### Preparation of (R)-(4-(6-chloro-3-((1-(2-(4,4-dimethylpiperidin-1-yl)-3,6-dimethyl-4-oxo-4H-chromen-8-yl)ethyl)amino)pyridin-2-yl)-2-fluorophenyl)boronic acid

The title compound was prepared as an off-white solid (210.1 mg, 361.13 μmol, 41.06% yield) according to the procedures described herein and the synthetic scheme, using [4-[6-chloro-3-[[(1R)-1-[2-(4,4-dimethyl-1-piperidyl)-3,6-dimethyl-4-oxo-chromen-8-yl]ethyl]amino]-2-pyridyl]-2-fluoro-phenyl]trifluoromethanesulfonate (600 mg, 879.6 μmol, 1.0 eq) and B₂neop₂ (794.8 mg, 3.52 mmol, 4.0 eq). ¹H NMR (DMSO-d₆, 400 MHz) δ 7.68 (t, J=6.8 Hz, 1H), 7.61 (s, 1H), 7.46 (d, J=7.6 Hz, 1H), 7.43 (s, 1H), 7.31 (d, J=10.4 Hz, 1H), 7.13 (d, J=8.8 Hz, 1H), 6.93 (d, J=8.8 Hz, 1H), 5.46 (d, J=7.2 Hz, 1H), 4.97-4.87 (m, 1H), 3.37-3.20 (m, 4H), 2.30 (s, 3H), 1.88 (s, 3H), 1.49 (d, J=6.8 Hz, 3H), 1.41-1.31 (m, 4H), 0.93 (s, 6H). MS (ESI): mass calcd. For C₃₁H₃₄BClFN₃O₄ 577.23, m/z found 578.2 [M+H]⁺. HPLC: 99.33 (220 nm), 99.44 (254 nm). Chiral purity: 98.92% ee.

### Example 88

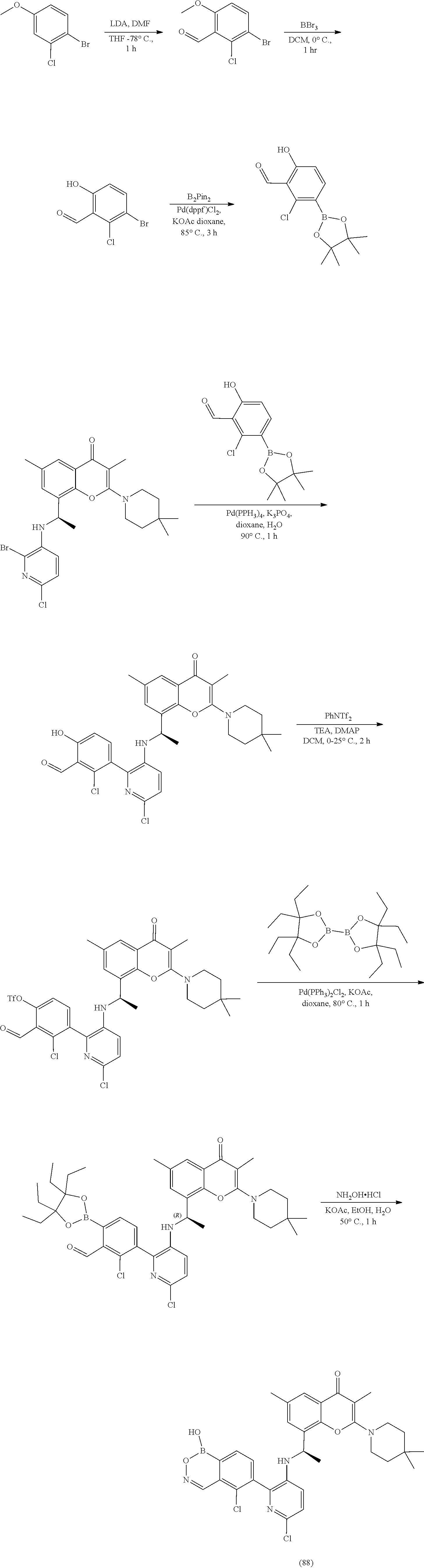

### Preparation of 3-bromo-2-chloro-6-methoxy-benzaldehyde

To a solution of 1-bromo-2-chloro-4-methoxy-benzene (10.00 g, 45.15 mmol, 1 eq) in THF (100 mL) was added LDA (2 M, 27 mL, 1.2 eq) at −78° C., the reaction mixture was stirred at −78° C. for 20 min. Then DMF (4.29 g, 58.70 mmol, 4.52 mL, 1.3 eq) was added dropwise to the above mixture at −78° C., the resulting reaction mixture was stirred at −78° C. for 40 min. The reaction mixture was quenched by addition sat.aq.NH₄Cl (50 mL) at 0° C. and extracted with EtOAc (30 mL×3). The combined organic layers were washed with brine (30 mL×2), dried over Na₂SO₄, filtered and concentrated under reduced pressure to give the title compound (10.00 g, 40.08 mmol, 88.77% yield) as a yellow oil. ¹H NMR (CDCl₃ 400 MHz.) δ 10.44 (s, 1H), 7.74 (d, J=9.2 Hz, 1H), 6.85 (d, J=8.8 Hz, 1H), 3.93 (s, 3H).

### Preparation of 3-bromo-2-chloro-6-hydroxy-benzaldehyde

To a solution of 3-bromo-2-chloro-6-methoxy-benzaldehyde (10.0 g, 40.08 mmol, 1 eq) in DCM (100 mL) was added BBr₃ (15.06 g, 60.12 mmol, 5.79 mL, 1.5 eq) at 0° C., the reaction mixture was stirred at 0° C. for 1 h. The reaction mixture was quenched by adding water (50 mL) at 0° C., and extracted with EtOAc (30 mL×3). The combined organic layers were washed with brine (10 mL×3), dried over Na₂SO₄, filtered and concentrated under reduced pressure to give the title compound (8.00 g, 33.98 mmol, 84.77% yield) as a yellow solid. ¹H NMR (CDCl₃ 400 MHz.) δ 12.03 (s, 1H), 10.44 (s, 1H), 7.71 (d, J=9.2 Hz, 1H), 6.85 (d, J=9.2 Hz, 1H).

### Preparation of 2-chloro-6-hydroxy-3-(4,4,5,5-tetramethyl-1,3,2-dioxaborolan-2-yl)benzaldehyde

To a solution of 3-bromo-2-chloro-6-hydroxy-benzaldehyde (1.20 g, 5.10 mmol, 1 eq) and B₂Pin₂ (1.42 g, 5.61 mmol, 1.1 eq) in dioxane (30 mL) were added Pd(dppf)Cl₂ (373 mg, 509.64 mol, 0.1 eq) and KOAc (1.00 g, 10.19 mmol, 2 eq) in turns at 25° C., the reaction mixture was heated to 85° C. and stirred at 85° C. for 3 h. The mixture was cooled to 25° C. and filtered. The filtrate was concentrated under reduced pressure and the residue was purified by column chromatography (SiO2, Petroleum ether/Ethyl acetate=10/1 to 5/1) to give the title compound (1.20 g, 4.25 mmol, 83.34% yield) as a yellow solid. ¹H NMR (CDCl₃ 400 MHz) δ 12.18 (s, 1H), 10.44 (s, 1H), 7.76 (d, J=8.4 Hz, 1H), 6.80 (d, J=8.8 Hz, 1H), 1.28 (s, 12H).

### Preparation of (R)-2-chloro-3-(6-chloro-3-((1-(2-(4,4-dimethylpiperidin-1-yl)-3,6-dimethyl-4-oxo-4H-chromen-8-yl)ethyl)amino)pyridin-2-yl)-6-hydroxybenzaldehyde

To a solution of (R)-8-(1-((2-bromo-6-chloropyridin-3-yl)amino)ethyl)-2-(4,4-dimethylpiperidin-1-yl)-3,6-dimethyl-4H-chromen-4-one (1.30 g, 2.51 mmol, 1 eq) and 2-chloro-6-hydroxy-3-(4,4,5,5-tetramethyl-1,3,2-dioxaborolan-2-yl)benzaldehyde (1.42 g, 5.01 mmol, 2 eq) in dioxane (4 mL) and H₂O (1 mL) were added Pd(PPh₃)₄ (289.5 mg, 250.54 μmol, 0.1 eq) and K₃PO₄ (1.60 g, 7.52 mmol, 3 eq) in turns at 25° C., the reaction mixture was heated to 90° C. and stirred at 90° C. for 1 h. The reaction mixture was cooled to 25° C. and filtered. The filtrate was concentrated under reduced pressure and the residue was purified by column chromatography (SiO₂, Petroleum ether/Ethyl acetate=3/1 to 1/1) to give the title compound (2.00 g, crude) as a white solid, which was used directly for next step without further purification.

### Preparation of (R)-3-chloro-4-(6-chloro-3-((1-(2-(4,4-dimethylpiperidin-1-yl)-3,6-dimethyl-4-oxo-4H-chromen-8-yl)ethyl)amino)pyridin-2-yl)-2-formylphenyl trifluoromethanesulfonate

To a solution of (R)-2-chloro-3-(6-chloro-3-((1-(2-(4,4-dimethylpiperidin-1-yl)-3,6-dimethyl-4-oxo-4H-chromen-8-yl)ethyl)amino)pyridin-2-yl)-6-hydroxybenzaldehyde (1.80 g, 3.03 mmol, 1 eq) and [N-(trifluoromethylsulfonyloxy)anilino]trifluoromethanesulfonate (1.18 g, 3.03 mmol, 1 eq) in DCM (20 mL) were added TEA (612.7 mg, 6.06 mmol, 842.82 μL, 2 eq) and DMAP (18.5 mg, 151.38 mol, 0.05 eq) in turns at 0° C., the reaction mixture was stirred at 25° C. for 2 h. The reaction mixture was quenched by adding water (20 mL) at 0° C. and extracted with DCM (20 mL×3). The combined organic layers were washed with brine (10 mL×2), dried over Na₂SO₄ and concentrated in vacuo. The residue was purified by column chromatography (SiO2, Petroleum ether/Ethyl acetate=5/1 to 3/1) to give the title compound (2.00 g, 2.75 mmol, 90.92% yield) as yellow oil. ¹H NMR (CDCl₃, 400 MHz) δ 10.46 (s, 1H), 7.82 (s, 1H), 7.69-7.65 (m, 1H), 7.42-7.40 (m, 1H), 7.32-7.30 (m, 1H), 7.04-7.02 (m, 1H), 6.61-6.59 (m, 1H), 4.89-4.87 (m, 1H), 4.07-4.02 (m, 1H), 3.33-3.30 (m, 4H), 2.28-2.27 (m, 3H), 1.99 (s, 3H), 1.48-1.45 (m, 7H), 0.97-0.96 (m, 6H).

### Preparation of (R)-2-chloro-3-(6-chloro-3-((1-(2-(4,4-dimethylpiperidin-1-yl)-3,6-dimethyl-4-oxo-4H-chromen-8-yl)ethyl)amino)pyridin-2-yl)-6-(4,4,5,5-tetraethyl-1,3,2-dioxaborolan-2-yl)benzaldehyde

A mixture of hypodiboric acid (3.80 g, 42.39 mmol, 1 eq), 3,4-diethylhexane-3,4-diol (14.77 g, 84.77 mmol, 2 eq) and KOAc (10.40 g, 105.97 mmol, 2.5 eq) in toluene (400 mL) at 25° C. was degassed and purged with N₂ for 3 times, and then the mixture was heated to 120° C. and stirred at 120° C. with Dean-Stark for 48 h. The reaction mixture was cooled to 25° C., filtered and the filtrate was concentrated in vacuo to give 4,4,4′,4′,5,5,5′,5′-octaethyl-2,2′-bi(1,3,2-dioxaborolane) (20.5 g, crude) as colorless oil, which was used directly for next step without further purification. ¹H NMR (CDCl₃, 400 MHz) δ 1.82-1.70 (m, 16H), 1.05-0.98 (m, 24H). To a solution of (R)-3-chloro-4-(6-chloro-3-((1-(2-(4,4-dimethylpiperidin-1-yl)-3,6-dimethyl-4-oxo-4H-chromen-8-yl)ethyl)amino)pyridin-2-yl)-2-formylphenyltrifluoromethanesulfonate (100.0 mg, 137.63 μmol, 1 eq), 4,4,4′,4′,5,5,5′,5′-octaethyl-2,2′-bi(1,3,2-dioxaborolane) (201.6 mg, 550.52 μmol, 4 eq) and KOAc (40.5 mg, 412.89 μmol, 3 eq) in dioxane (4 mL) was added Pd(PPh₃)₂Cl₂ (10.0 mg, 13.76 μmol, 0.1 eq) in one portion at 25° C., the resulting reaction mixture was heated to 80° C. and stirred at 80° C. for 1 h. The reaction mixture was filtered and the filtrate was concentrated under reduced pressure to give a residue. The residue was directly purified by prep-HPLC (column: Waters Xbridge BEH C18 100×25 mm×10 um; mobile phase: [H₂O (10 mM NH₄HCO₃)-AC□]; gradient: 50%-80% B over 8.0 min) to give the title compound (50 mg, 65.74 μmol, 47.76% yield) as a yellow solid.

### Preparation of (R)-8-(1-((6-chloro-2-(5-chloro-1-hydroxy-JH-benzo[d][1,2,6]oxazabor inin-6-yl)pyridin-3-yl)amino)ethyl)-2-(4,4-dimethylpiperidin-1-yl)-3,6-dimethyl-4H-chrome n-4-one

To a solution of (R)-5-(6-chloro-3-((1-(2-(4,4-dimethylpiperidin-1-yl)-3,6-dimethyl-4-oxo-4H-chromen-8-yl)ethyl)amino)pyridin-2-yl)-2-(4,4,5,5-tetraethyl-1,3,2-dioxaborolan-2-yl)benzal dehyde (50 mg, 70.97 μmol, 1 eq) and NH₂OH·HCl (5 mg, 70.97 μmol, 1 eq) in EtOH (2 mL)/H₂O (1 mL) was added KOAc (7 mg, 70.97 μmol, 1 eq) in one portion at 25° C., the reaction mixture was heated to 50° C. for 1 h. The reaction mixture was cooled to 25° C. and directly purified by prep-HPLC (column: Waters Xbridge BEH C18 100×25 mm×10 um; mobile phase: [H₂O (10 mM NH₄HCO₃)-AC□]; gradient: 20%-55% B over 8.0 min) to give the title compound (6.10 mg, 9.85 μmol, 13.88% yield) as a white solid. ¹H NMR (MeOD, 400 MHz) δ 8.53 (d, J=4.8 Hz, 1H), 7.80-7.70 (m, 2H), 7.56-7.45 (m, 1H), 7.43-7.35 (m, 1H), 7.20-7.10 (m, 1H), 7.02-6.85 (m, 1H), 5.10-4.95 (m, 1H), 3.55-3.33 (m, 4H), 2.37 (s, 3H), 2.05 (s, 3H), 1.57-1.49 (s, 7H), 1.10-1.01 (m, 6H). MS (ESI): mass calcd. For C₃₂H₃₃BCl₂N₄O₄ 618.20, m/z found 619.2 [M+H]⁺. HPLC: 90.77% (220 nm), 92.56% (254 nm). Chiral purity: 97.08% ee

### Example 89

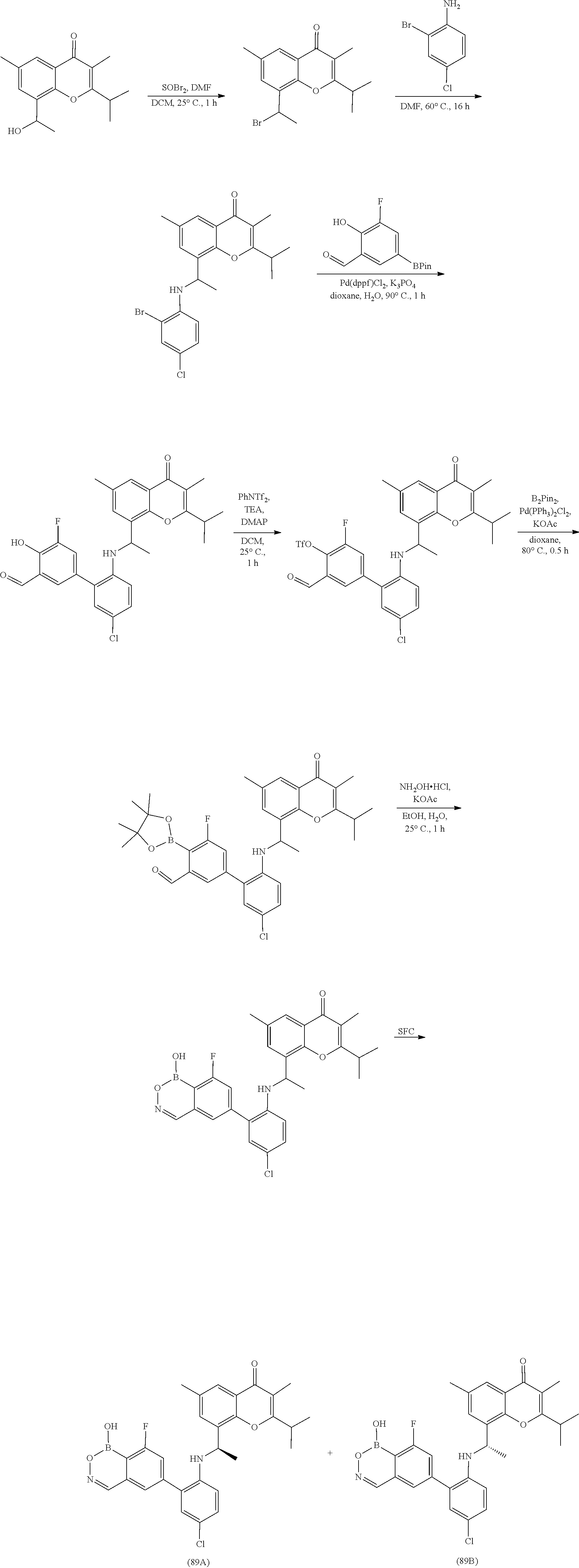

### Preparation of 8-(1-bromoethyl)-2-isopropyl-3,6-dimethyl-4H-chromen-4-one

To a solution of 8-(1-hydroxyethyl)-2-isopropyl-3,6-dimethyl-chromen-4-one (2.20 g, 8.45 mmol, 1 eq) and DMF (31 mg, 422.54 μmol, 32.51 μL, 0.05 eq) in DCM (20 mL) was added SOBr₂ (2.28 g, 10.99 mmol, 851.18 μL, 1.3 eq) dropwise at 0° C., the reaction mixture was warmed to 25° C. and stirred at 25° C. for 1 h. The reaction mixture was poured into ice H₂O (20 mL) at 0° C., and then extracted with DCM (20 mL×3). The combined organic layers were washed with sat.aq NaHCO₃ until pH=8, then washed with brine (40 mL), dried over Na₂SO₄, filtered and concentrated in vacuo to give the title compound (3.20 g, 93% yield) as a light brown solid. ¹H NMR (DMSO-d₆, 400 MHz) δ 7.93 (d, J=1.2 Hz, 1H), 7.52 (d, J=2.0 Hz, 1H), 5.66-5.61 (m, 1H), 3.27-3.20 (m, 1H), 2.38 (s, 3H), 2.09-2.08 (m, 3H), 2.03-2.00 (m, 3H), 1.35-1.30 (m, 6H).

### Preparation of 8-(1-((2-bromo-4-chlorophenyl)amino)ethyl)-2-isopropyl-3,6-dimethyl-4H-chromen-4-one

To a solution of 8-(1-bromoethyl)-2-isopropyl-3,6-dimethyl-chromen-4-one (800 mg, 2.48 mmol, 1 eq) in DMF (10 mL) was added 2-bromo-4-chloro-aniline (1.12 g, 5.45 mmol, 2.2 eq), the reaction mixture was heated to 60° C. and stirred at 60° C. for 16 h. The reaction mixture was cooled to 25° C. The reaction mixture was poured into ice-water (30 mL) at 0° C. under N₂ and then extracted with EtOAc (10 mL×3). The combined organic phases were washed with brine (20 mL), dried over Na₂SO₄, filtered and concentrated under reduced pressure to give a residue. The residue was purified by flash silica gel chromatography (Biotage®; 12 g SepaFlash® Silica Flash Column, Eluent of 0~15% Ethyl acetate/Petroleum ether gradient @60 mL/min). The eluent was concentrated under reduced pressure to give the title compound (950 mg, 2.12 mmol, 85.53% yield) as a light yellow solid. ¹H NMR (DMSO-d₆, 400 MHz) δ 7.89 (d, J=1.2 Hz, 1H), 7.44 (d, J=2.4 Hz, 1H), 7.38 (d, J=2.0 Hz, 1H), 6.96 (d, J=2.4 Hz, 1H), 6.20 (d, J=8.8 Hz, 1H), 5.07-5.02 (m, 1H), 3.36-3.29 (m, 1H), 2.38 (s, 3H), 2.12 (s, 3H), 1.67-1.66 (m, 3H), 1.38-1.36 (m, 6H).

### Preparation of 5′-chloro-5-fluoro-4-hydroxy-2′-((1-(2-isopropyl-3,6-dimethyl-4-oxo-4H-chromen-8-yl)ethyl)amino)-[1,1′-biphenyl]-3-carbaldehyde

A mixture of 8-[1-(2-bromo-4-chloro-anilino)ethyl]-2-isopropyl-3,6-dimethylchromen-4-one (900 mg, 2.01 mmol, 1 eq), 3-fluoro-2-hydroxy-5-(4,4,5,5-tetramethyl-1,3,2-dioxaborola-2-yl)benzaldehyde (801 mg, 3.01 mmol, 1.5 eq), K₃PO₄ (1.28 g, 6.02 mmol, 3 eq) and Pd(dppf)Cl₂ (146.74 mg, 200.54 μmol, 0.1 eq) in dioxane (10 mL)/H₂O (2 mL) was degassed and purged with N₂ for 3 times, and then the reaction mixture was heated to 90° C. and stirred at 90° C. for 1 h under N₂ atmosphere. The reaction mixture was cooled to 25° C., diluted with H₂O (10 mL) and extracted with EtOAc (10 mL×2). The combined organic layers were dried over Na₂SO₄, filtered and concentrated under reduced pressure to give a residue. The residue was purified by flash silica gel chromatography (Biotage®; 12 g SepaFlash® Silica Flash Column, Eluent of 0~45% Ethyl acetate/Petroleum ether gradient @60 mL/min). The eluent was concentrated under reduced pressure to give the title compound (800 mg, 1.57 mmol, 78.53% yield) as a yellow solid. ¹H NMR (DMSO-d₆, 400 MHz) δ 11.07 (s, 1H), 10.00 (d, J=1.6 Hz, 1H), 7.90 (d, J=1.2 Hz, 1H), 7.51-7.48 (m, 2H), 7.32 (s, 1H), 7.07-7.05 (m, 2H), 6.39-6.36 (m, 1H), 5.05-5.00 (m, 1H), 3.33-3.26 (m, 1H), 2.38 (s, 3H), 2.11 (s, 3H), 1.55-1.53 (m, 3H), 1.35-1.34 (m, 3H), 1.30-1.29 (m, 3H).

### Preparation of 5′-chloro-3-fluoro-5-formyl-2′-((1-(2-isopropyl-3,6-dimethyl-4-oxo-4H-chromen-8-yl)ethyl)amino)-[1,1′-biphenyl]-4-yl trifluoromethanesulfonate

To a solution of 5-[5-chloro-2-[1-(2-isopropyl-3,6-dimethyl-4-oxo-chromen-8-yl)ethylamino]phenyl]-3-fluoro-2-hydroxy-benzaldehyde (700 mg, 1.38 mmol, 1 eq) and 1,1,1-trifluoro-N-phenyl-N-(trifluoromethylsulfonyl)methanesulfonamide (985 mg, 2.76 mmol, 2 eq) in DCM (7 mL) were added DMAP (16.83 mg, 137.80 μmol, 0.1 eq) and TEA (278.88 mg, 2.76 mmol, 383.60 μL, 2 eq) in turns at 25° C., the reaction mixture was stirred at 25° C. for 1 h. The reaction mixture was quenched with H₂O (10 mL) at 25° C. and extracted with DCM (10 mL×2). The combined organic layers were washed with brine and concentrated under reduced pressure. The residue was purified by flash silica gel chromatography (Biotage®; 20 g SepaFlash® Silica Flash Column, Eluent of 0~20% Ethyl acetate/Petroleum ether gradient @30 L/min) to give the title compound (800 mg, 1.25 mmol, 90.70% yield) as yellow oil. ¹H NMR (DMSO-d₆, 400 MHz) δ 10.10 (d, J=1.6 Hz, 1H), 8.18-8.15 (m, 2H), 7.68 (d, J=8.0 Hz, 1H), 7.55 (d, J=2.0 Hz, 1H), 7.22 (d, J=2.8 Hz, 1H), 7.16-7.13 (m, 1H), 6.43 (d, J=8.8 Hz, 1H), 5.71 (d, J=7.2 Hz, 1H), 5.02-4.95 (m, 1H), 2.50 (s, 1H), 2.34 (s, 3H), 1.99 (s, 3H), 1.48 (d, J=6.8 Hz, 3H), 1.24 (d, J=6.8 Hz, 3H).

### Preparation of 5′-chloro-5-fluoro-2′-((1-(2-isopropyl-3,6-dimethyl-4-oxo-4H-chromen-8-yl)ethyl)amino)-4-(4,4,5,5-tetramethyl-1,3,2-dioxaborolan-2-yl)-[1,1′-biphenyl]-3-carbaldehyde

A mixture of [4-[5-chloro-2-[1-(2-isopropyl-3,6-dimethyl-4-oxo-chromen-8-yl)ethylamino]phenyl]-2-fluoro-6-formyl-phenyl]trifluoromethanesulfonate (600 mg, 937.44 μmol, 1 eq), B₂Pin₂ (715 mg, 2.81 mmol, 3 eq), Pd(PPh₃)₂Cl₂ (66 mg, 93.74 μmol, 0.1 eq) and KOAc (184 mg, 1.87 mmol, 2 eq) in dioxane (6 mL) was degassed and purged with N₂ for 3 times at 25° C., and then the reaction mixture was heated to 80° C. and stirred at 80° C. for 30 min under N₂ atmosphere. The reaction mixture was cooled to room temperature, filtered and the filtrate was concentrated under reduced pressure. The residue was triturated with MTBE (50 mL) and the filtrate was concentrated under reduced pressure to give the title compound (1.2 g, crude) as a black solid, which was used directly for next step without further purification.

### Preparation of 8-(1-((4-chloro-2-(8-fluoro-1-hydroxy-JH-benzo[d][1,2,6]oxazabor inin-6-yl)phenyl)amino)ethyl)-2-isopropyl-3,6-dimethyl-4H-chromen-4-one

To a solution of 5-[5-chloro-2-[1-(2-isopropyl-3,6-dimethyl-4-oxo-chromen-8-yl) ethylamino]phenyl]-3-fluoro-2-(4,4,5,5-tetramethyl-1,3,2-dioxaborolan-2-yl)benzaldehyde (1.00 g, 1.62 mmol, 1 eq) in EtOH (10 mL)/H₂O (2.5 mL) was added hydroxylamine;hydrochloride (169 mg, 2.43 mmol, 1.5 eq) and KOAc (318 mg, 3.24 mmol, 2 eq) in one portion at 0° C., the reaction mixture was warmed to 25° C. and stirred at 25° C. for 1 h. The reaction mixture diluted with H₂O (6 mL) and white solid formed. The reaction suspension was filtered and the filter cake was purified by prep-HPLC (column: Waters Xbridge BEH C18 100×30 mm×10 um; mobile phase: [H₂O (10 mM NH₄HCO₃)-ACN]; gradient:45%-85% B over 8.0 min) to give the title compound (130 mg, 244.00 μmol, 15.08% yield) as a white solid.

### Preparation of 8-[(1S)-1-[4-chloro-2-(8-fluoro-1-hydroxy-2,3,1-benz oxazaborinin-6-yl)anilino]ethyl]-2-isopropyl-3,6-dimethyl-chromen-4-one and 8-[(1R)-1-[4-chloro-2-(8-fluoro-1-hydroxy-2,3,1-benzoxazaborinin-6-yl)anilino]ethyl]-2-isopropyl-3,6-dimethyl-chromen-4-one

8-[1-[4-chloro-2-(8-fluoro-1-hydroxy-2,3,1-benzoxazaborinin-6-yl)anilino]ethyl]-2-isopropyl-3,6-dimethyl-chromen-4-one (130 mg) was purified by SFC (column: DAICEL CHIRALPAK AD (250 mm×30 mm×10 um); mobile phase: [CO2-IPA]; B %:33%, isocratic elution mode) to give the title compound, Isomer 1 (36.2 mg, 67.9 μmol, 27.8% yield) as a white solid and the title compound, Isomer 2 (47.6 mg, 89.3 μmol, 36.6% yield) as a white solid. Isomer 1: ¹H NMR (DMSO-d₆, 400 MHz) δ 9.34 (s, 1H), 8.72 (d, J=1.6 Hz, 1H), 7.74 (s, 1H), 7.67 (s, 1H), 7.64 (s, 1H), 7.54 (s, 1H), 7.17-7.09 (m, 2H), 6.45 (d, J=9.6 Hz, 1H), 5.50 (d, J=7.2 Hz, 1H), 5.04-4.85 (m, 1H), 3.30-3.26 (m, 1H), 2.34 (s, 3H), 1.99 (s, 3H), 1.46 (d, J=6.8 Hz, 3H), 1.30-1.24 (m, 3H), 1.23-1.16 (m, 3H) MS (ESI): mass calcd. For C₂₉H₂₇BClFN₂O₄ 532.17, m/z found 533.2 [M+H]⁺. HPLC: 98.27% (220 nm), 99.02% (254 nm). Chiral purity: 100 ee %. Isomer 2: ¹H NMR (DMSO-d₆, 400 MHz) δ 9.36 (s, 1H), 8.72 (d, J=2.0 Hz, 1H), 7.75 (s, 1H), 7.67 (s, 1H), 7.64 (s, 1H), 7.54 (d, J=1.6 Hz, 1H), 7.19-7.07 (m, 2H), 6.50-6.39 (m, 1H), 5.50 (d, J=7.2 Hz, 1H), 5.03-4.92 (m, 1H), 3.31-3.26 (m, 1H), 2.34 (s, 3H), 1.99 (s, 3H), 1.46 (d, J=6.4 Hz, 3H), 1.31-1.25 (m, 3H), 1.23-1.17 (m, 3H). MS (ESI): mass calcd. For C₂₉H₂₇BClFN₂O₄ 532.17, m/z found 533.2 [M+H]⁺. HPLC: 97.79% (220 nm), 98.15% (254 nm). Chiral purity: 98.76 ee %

### Example 90

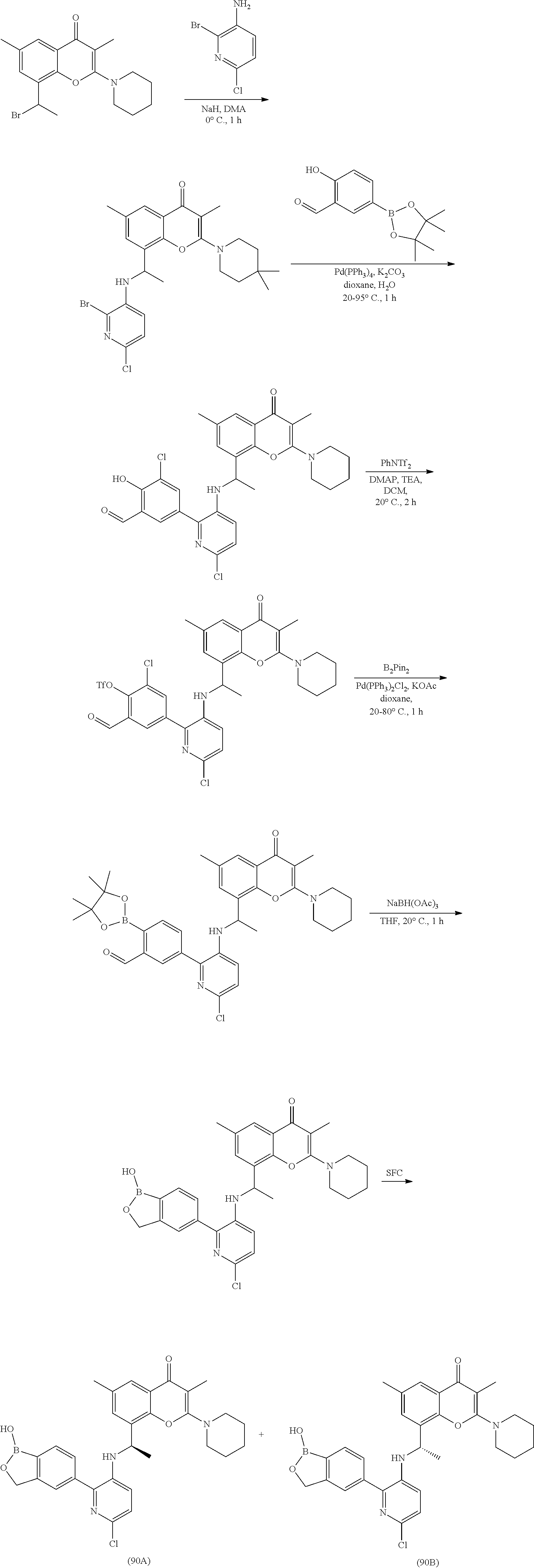

### Preparation of 8-(1-((2-bromo-6-chloropyridin-3-yl)amino)ethyl)-3,6-dimethyl-2-(piperidin-1-yl)-4H-chromen-4-one

To a solution of 2-bromo-6-chloropyridin-3-amine (2.05 g, 9.88 mmol, 1.2 eq) in DMA (40 mL) was added NaH (491 mg, 12.2 mmol, 1.49 eq) in portions at 0° C. After addition, the reaction mixture was stirred at 0° C. for 0.5 h, and then a solution of 8-(1-bromoethyl)-3,6-dimethyl-2-(piperidin-1-yl)-4H-chromen-4-one (3.00 g, 8.24 mmol, 1 eq) in DMA (40 mL) was added dropwise to the above reaction mixture at 0° C. The resulting reaction mixture was stirred at 0° C. for 0.5 h. The reaction mixture was quenched by adding water (150 mL) at 0° C., and then the resulting suspension was directly filtered. The filter cake was washed with cold water and dried in vacuo to give the title compound (3.00 g, 6.11 mmol, 74.22% yield) as a red solid. ¹H NMR (CDCl₃, 400 MHz) δ 7.90 (s, 1H), 7.28-7.27 (m, 1H), 6.97-6.95 (m, 1H), 6.47 (d, J=8.4 Hz, 1H), 4.94-4.88 (m, 1H), 4.82-4.81 (m, 1H), 3.40-3.38 (m, 4H), 2.37 (s, 3H), 2.06 (s, 3H), 1.72-1.68 (m, 6H), 1.60 (s, 3H).

### Preparation of 5-(6-chloro-3-((1-(3,6-dimethyl-4-oxo-2-(piperidin-1-yl)-4H-chromen-8-yl)ethyl)amino)pyridin-2-yl)-2-hydroxybenzaldehyde

A mixture of 8-(1-((2-bromo-6-chloropyridin-3-yl)amino)ethyl)-3,6-dimethyl-2-(piperidin-1-yl)-4H-chromen-4-one (500 mg, 1.02 mmol, 1 eq), 2-hydroxy-5-(4,4,5,5-tetramethyl-1,3,2-dioxaborolan-2-yl)benzaldehyde (379 mg, 1.53 mmol, 1.5 eq), Pd(PPh₃)₄ (117 mg, 101 μmol, 0.1 eq) and K₂CO₃ (281 mg, 2.04 mmol, 2 eq) in H₂O (0.5 mL)/dioxane (5 mL) was degassed and purged with N₂ for 3 times at 20° C., and then the mixture was heated to 95° C. and stirred at 95° C. for 1 h under N₂ atmosphere. The reaction mixture was filtered and the filtrate was concentrated under reduced pressure to give a residue. The residue was purified by flash silica gel chromatography (Biotage®; 12 g SepaFlash® Silica Flash Column, Eluent of 30~40% Ethyl acetate/Petroleum ether gradient @80 mL/min) to give the title compound (500 mg, 939.80 μmol, 92.25% yield) as a yellow solid. MS (ESI): mass calcd. 531.19, m/z found 532.1 [M+H]⁺.

### Preparation of 4-(6-chloro-3-((1-(3,6-dimethyl-4-oxo-2-(piperidin-1-yl)-4H-chromen-8-yl)ethyl)amino)pyridin-2-yl)-2-formylphenyl trifluoromethanesulfonate

A mixture of 5-(6-chloro-3-((1-(3,6-dimethyl-4-oxo-2-(piperidin-1-yl)-4H-chromen-8-yl)ethyl)amino)pyridin-2-yl)-2-hydroxybenzaldehyde (600 mg, 1.13 mmol, 1 eq), PhNTf₂ (805 mg, 2.26 mmol, 2 eq), TEA (228 mg, 2.26 mmol, 313 μL, 2 eq) and DMAP (13 mg, 112 μmol, 0.1 eq) in DCM (6 mL) was degassed and purged with N₂ for 3 times at 20° C., and then the mixture was stirred at 20° C. for 1 h under N₂ atmosphere. The reaction mixture was filtered and the filtrate was concentrated under reduced pressure to give a residue. The residue was purified by flash silica gel chromatography (Biotage®(R; 20 g SepaFlash® Silica Flash Column, Eluent of 30~35% Ethyl acetate/Petroleum ether gradient @80 mL/min) to give the title compound (600 mg, 903.49 μmol, 80.11% yield) as a yellow solid. ¹H NMR (CDCl₃, 400 MHz) δ 10.35 (s, 1H), 8.44 (d, J=2.0 Hz, 1H), 8.15-8.12 (m, 1H), 7.92 (s, 1H), 7.60-7.58 (m, 1H), 7.33-7.29 (m, 1H), 7.06-7.03 (m, 1H), 6.69-6.67 (m, 1H), 4.96-4.91 (m, 1H), 4.39-4.38 (m, 1H), 3.37-3.37 (m, 4H), 2.38 (s, 3H), 2.08 (s, 3H), 1.71-1.71 (m, 6H), 1.59 (d, J=6.8 Hz, 3H).

### Preparation of 5-(6-chloro-3-((1-(3,6-dimethyl-4-oxo-2-(piperidin-1-yl)-4H-chromen-8-yl)ethyl)amino)pyridin-2-yl)-2-(4,4,5,5-tetramethyl-1,3,2-dioxaborolan-2-yl)benzaldehyde

A mixture of 4-(6-chloro-3-((1-(3,6-dimethyl-4-oxo-2-(piperidin-1-yl)-4H-chromen-8-yl)ethyl)amino)pyridin-2-yl)-2-formylphenyl trifluoromethanesulfonate (250 mg, 376 μmol, 1 eq), B₂Pin₂ (477 mg, 1.88 mmol, 5 eq), KOAc (73 mg, 752 μmol, 2 eq) and Pd(PPh₃)₂Cl₂ (26 mg, 37 μmol, 0.1 eq) in dioxane (3 mL) was degassed and purged with N₂ for 3 times at 20° C., and then the mixture was heated to 80° C. and stirred at 80° C. for 1 h under N₂ atmosphere. The reaction mixture was filtered and the filtrate was concentrated under reduced pressure to give a residue. The residue was purified by flash silica gel chromatography (Biotage®; 12 g SepaFlash® Silica Flash Column, Eluent of 60~70% Ethyl acetate/Petroleum ether gradient @50 mL/min) to give the title compound (0.3 g, crude) as yellow oil, which was used directly for next step without further purification. ¹H NMR (CDCl₃, 400 MHz) δ 10.62 (s, 1H), 8.34 (s, 1H), 8.05-8.03 (m, 1H), 7.96-7.94 (m, 1H), 7.89 (d, J=1.2 Hz, 1H), 7.27-7.26 (m, 1H), 7.01 (d, J=8.8 Hz, 1H), 6.64 (d, J=8.8 Hz, 1H), 4.89-4.87 (m, 1H), 4.47-4.46 (m, 1H), 3.31-3.28 (m, 4H), 2.37 (s, 3H), 2.06 (s, 3H), 1.68-1.68 (m, 6H), 1.53 (d, J=6.4 Hz, 3H), 1.43 (s, 12H).

### Preparation of 8-(1-((6-chloro-2-(1-hydroxy-1,3-dihydrobenzo[c][1,2]oxaborol-5-yl) pyridine-3-yl)amino)ethyl)-3,6-dimethyl-2-(piperidin-1-yl)-4H-chromen-4-one

To a solution of 5-(6-chloro-3-((1-(3,6-dimethyl-4-oxo-2-(piperidin-1-yl)-4H-chromen-8-yl) ethyl)amino)pyridin-2-yl)-2-(4,4,5,5-tetramethyl-1,3,2-dioxaborolan-2-yl)benzaldehyde (450 mg, 700 μmol, 1 eq) in THF (5 mL) was added NaBH(OAc)₃ (742 mg, 3.50 mmol, 5 eq) in portions at 20° C., and then the reaction mixture was stirred at 20° C. for 1 h under N₂ atmosphere. The reaction mixture was quenched with H₂O (20 mL) at 20° C. and extracted with EtOAc (15 mL×2). The combined organic layers were washed with brine, dried over anhydrous Na₂SO₄, filtered and concentrated under reduced pressure to give a residue. The residue was purified by prep-HPLC (column: 2_Phenomenex Gemini C18 75×40 mm×3 um; mobile phase: [H₂O (10 mM NH₄HCO₃)-AC□]; gradient: 25%-65% B over 8.0 min) to give the title compound (80 mg, 147 μmol, 20.99% yield) as a white solid. ¹H NMR (DMSO-d₆, 400 MHz) δ 9.31 (s, 1H), 7.86-7.84 (m, 1H), 7.67-7.65 (m, 2H), 7.61-7.61 (m, 1H), 7.45 (d, J=2.0 Hz, 1H), 7.16-7.14 (m, 1H), 6.97-6.95 (m, 1H), 5.51-5.50 (m, 1H), 5.07-5.03 (m, 2H), 4.99-4.90 (m, 1H), 3.24-3.15 (m, 4H), 2.31 (s, 3H), 1.88 (s, 3H), 1.56 (s, 6H), 1.50 (d, J=6.4 Hz, 3H).

### Preparation of (S)-8-(1-((6-chloro-2-(1-hydroxy-1,3-dihydrobenzo[c][1,2]oxaborol-5-yl)pyridin-3-yl)amino)ethyl)-3,6-dimethyl-2-(piperidin-1-yl)-4H-chromen-4-one & (R)-8-(1-((6-chloro-2-(1-hydroxy-1,3-dihydrobenzo[c][1,2]oxaborol-5-yl)pyridin-3-yl)amino)ethyl)-3,6-dimethyl-2-(piperidin-1-yl)-4H-chromen-4-one

8-(1-((6-chloro-2-(1-hydroxy-1,3-dihydrobenzo[c][1,2]oxaborol-5-yl)pyridine-3-yl)amino)ethyl)-3,6-dimethyl-2-(piperidin-1-yl)-4H-chromen-4-one (80 mg, 147 μmol, 1 eq) was purified by SFC (column: DAICEL CHIRALCEL OD (250 mm×30 mm×10 um); mobile phase: [CO₂—IPA (0.1% NH₃H₂O)]; B %: 50%, isocratic elution mode) to give the title compound, Isomer 1 (20.4 mg. 37.19 gmol, 25.28% yield) as a white solid and the title compound, Isomer 2 (29.9 mg, 54.38 μmol, 36.97% yield) as a white solid. Isomer 1: ¹H NMR (DMSO-d₆, 400 MHz) δ 9.29 (s, 1H), 7.86-7.84 (m, 1H), 7.67-7.65 (m, 2H), 7.62-7.62 (m, 1H), 7.45 (d, J=2.0 Hz, 1H), 7.16-7.14 (m, 1H), 6.98-6.95 (m, 1H), 5.51-5.49 (m, 1H), 5.03 (s, 2H), 4.95-4.89 (m, 1H), 3.21-3.16 (m, 4H), 2.32 (s, 3H), 1.88 (s, 3H), 1.57 (s, 6H), 1.50 (d, J=6.8 Hz, 3H). MS (ESI): mass calcd. For C₃₀H₃₁BClN₃O₄ 543.86, m/z found 544.1 [M+H]⁺. HPLC: 99.14% (220 nm), 99.17% (254 nm). Chiral purity: 100 ee %. Isomer 2: ¹H NMR (DMSO-d₆, 400 MHz) δ 9.29 (s, 1H), 7.86-7.84 (m, 1H), 7.67-7.66 (m, 2H), 7.61-7.61 (m, 1H), 7.45 (d, J=2.4 Hz, 1H), 7.16-7.14 (m, 1H), 6.97-6.95 (m, 1H), 5.52-5.50 (m, 1H), 5.03 (s, 2H), 4.95-4.88 (m, 1H), 3.24-3.15 (m, 4H), 2.31 (s, 3H), 1.88 (s, 3H), 1.57 (s, 6H), 1.50 (d, J=6.8 Hz, 3H). MS (ESI): mass calcd. For C₃₀H₃₁BClN₃O₄ 543.86, m/z found 544.1 [M+H]⁺. HPLC: 98.92% (220 nm), 99.37% (254 nm). Chiral purity: 98.64 ee %

### Example 91

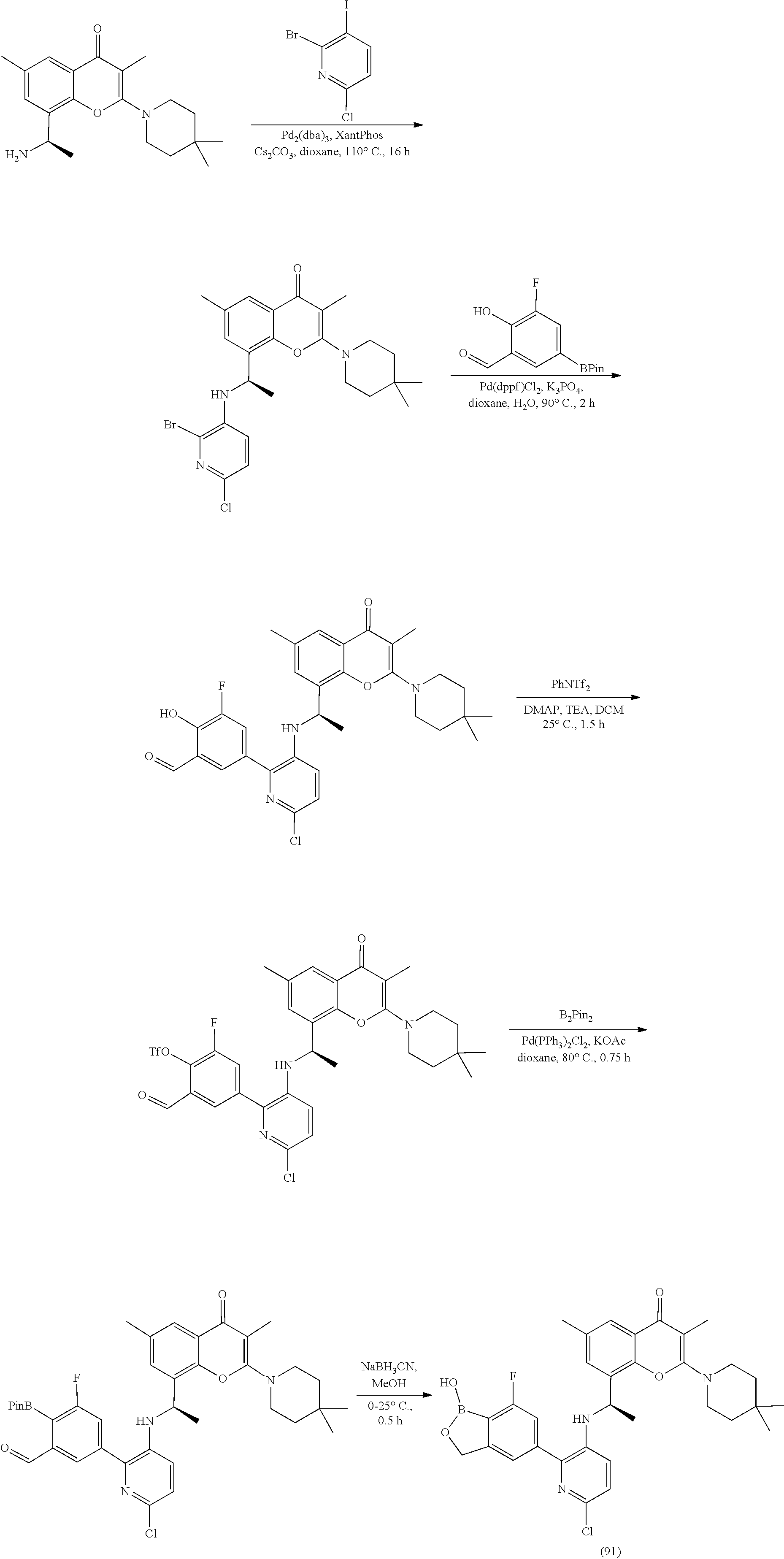

### Preparation of 8-[(1R)-1-[(2-bromo-6-chloro-3-pyridyl)amino]ethyl]-2-(4,4-dimethyl-1-piperidyl)-3,6-dimethyl-chromen-4-one

The title compound was prepared as an orange gum (3.35 g, 6.46 mmol, 70.69% yield) according to the similar procedure in Example 78, using 8-[(1R)-1-aminoethyl]-2-(4,4-dimethyl-1-piperidyl)-3,6-dimethyl-chromen-4-one (3.00 g, 9.13 mmol, 1 eq) and 2-bromo-6-chloro-3-iodo-pyridine (4.36 g, 13.70 mmol, 1.5 eq). ¹H NMR (DMSO-d₆, 400 MHz) δ 7.64 (s, 1H), 7.42 (d, J=2.0 Hz, 1H), 7.20 (d, J=8.4 Hz, 1H), 6.82 (d, J=8.4 Hz, 1H), 5.75 (s, 1H), 5.05-4.98 (m, 1H), 3.39 (t, J=5.6 Hz, 4H), 2.32 (s, 3H), 1.91 (s, 3H), 1.63 (d, J=6.8 Hz, 3H), 1.44 (t, J=5.6 Hz, 4H), 0.98 (s, 6H).

### Preparation of 5-[6-chloro-3-[[(1R)-1-[2-(4,4-dimethyl-1-piperidyl)-3,6-dimethyl-4-oxo-chromen-8-yl]ethyl]amino]-2-pyridyl]-3-fluoro-2-hydroxy-benzaldehyde

The title compound was prepared as an orange solid (483 mg, 835.54 μmol, 43.35% yield) according to the procedures described herein and the synthetic scheme, using 8-[(1R)-1-[(2-bromo-6-chloro-3-pyridyl)amino]ethyl]-2-(4,4-dimethyl-1-piperidyl)-3,6-dimethyl-chromen-4-one (1.00 g, 1.93 mmol, 1 eq) and 3-fluoro-2-hydroxy-5-(4,4,5,5-tetramethyl-1,3,2-dioxaborolan-2-yl)benzaldehyde (769 mg, 2.89 mmol, 1.5 eq). ¹H NMR (DMSO-d₆, 400 MHz) δ 10.32 (s, 1H), 7.87-7.83 (m, 2H), 7.62 (s, 1H), 7.45 (s, 1H), 7.12 (d, J=8.8 Hz, 1H), 6.90 (d, J=8.8 Hz, 1H), 5.82 (d, J=7.2 Hz, 1H), 4.95-4.89 (m, 1H), 3.31-3.28 (m, 4H), 2.31 (s, 3H), 1.90 (s, 3H), 1.51 (d, J=6.4 Hz, 3H), 1.42 (t, J=5.6 Hz, 4H), 0.97 (s, 6H).

### Preparation of (R)-4-(6-chloro-3-((1-(2-(4,4-dimethylpiperidin-1-yl)-3,6-dimethyl-4-oxo-4H-chromen-8-yl)ethyl)amino)pyridin-2-yl)-2-fluoro-6-formylphenyl trifluoromethanesulfonate

The title compound was prepared as an orange solid (755 mg, 1.06 mmol, 80.87% yield) according to the procedures described herein and the synthetic scheme, using 5-[6-chloro-3-[[(1R)-1-[2-(4,4-dimethyl-1-piperidyl)-3,6-dimethyl-4-oxo-chromen-8-yl]ethyl]amino]-2-pyridyl]-3-fluoro-2-hydroxy-benzaldehyde (760 mg, 1.31 mmol, 1 eq) and PhNTf₂ (705 mg, 1.97 mmol, 1.5 eq). ¹H NMR (DMSO-d₆, 400 MHz) δ 10.13 (s, 1H), 8.32-8.27 (m, 2H), 7.63 (s, 1H), 7.44 (s, 1H), 7.22 (d, J=7.6 Hz, 1H), 6.92 (d. J=8.8 Hz, 1H), 6.30 (d, J=6.0 Hz, 1H), 4.99-4.93 (m, 1H), 3.40-3.37 (m, 4H), 2.31 (s, 3H), 1.92 (s, 3H), 1.54 (d, J=6.8 Hz, 3H), 1.45 (t, J=5.6 Hz, 4H), 0.98 (s, 6H).

### Preparation of 5-[6-chloro-3-[[(1R)-1-[2-(4,4-dimethyl-1-piperidyl)-3,6-dimethyl-4-oxo-chromen-8-yl]ethyl]amino]-2-pyridyl]-3-fluoro-2-(4,4,5,5-tetramethyl-1,3,2-dioxaborolan-2-yl)benzaldehyde

The title compound was prepared as a brown solid (1.5 g, crude) according to the procedures described herein and the synthetic scheme, using [4-[6-chloro-3-[[(1R)-1-[2-(4,4-dimethyl-1-piperidyl)-3,6-dimethyl-4-oxo-chromen-8-yl]eth yl]amino]-2-pyridyl]-2-fluoro-6-formyl-phenyl]trifluoromethanesulfonate (700 mg, 985.73 μmol, 1 eq) and B₂Pin₂ (751 mg, 2.96 mmol, 3 eq). The product was used directly for next step without further purification.

### Preparation of 8-[(1R)-1-[[6-chloro-2-(7-fluoro-1-hydroxy-3H-2,1-benzoxaborol-5-yl)-3-pyridyl]amino]ethyl]-2-(4,4-dimethyl-1-piperidyl)-3,6-dimethyl-chromen-4-one

To a solution of 5-[6-chloro-3-[[(1R)-1-[2-(4,4-dimethyl-1-piperidyl)-3,6-dimethyl-4-oxo-chromen-8-yl]ethyl]amino]-2-pyridyl]-3-fluoro-2-(4,4,5,5-tetramethyl-1,3,2-dioxaborolan-2-yl)benzaldehyde (1.10 g, 1.60 mmol, 1 eq) in MeOH (11 mL) was added NaBH₃CN (1501 mg, 2.40 mmol, 1.5 eq) in one portion at 0° C., the reaction mixture was allowed to warm to 25° C. and stirred at 25° C. for 0.5 h. The reaction mixture was quenched by adding water (15 mL) and extracted with EtOAc (10 mL×2). The combined organic layers were washed with brine (20 mL), dried over Na₂SO₄, filtered and concentrated under reduced pressure. The residue was purified by prep-HPLC (column: Waters Xbridge BEH C18 100×30 mm×10 um; mobile phase: [H₂O (10 mM NH₄HCO₃)-AC□]; gradient: 60%-90% B over 8.0 min) to give the title compound (63.2 mg, 105.43 μmol, 6.59% yield) as an off-white solid. ¹H NMR (DMSO-d₆, 400 MHz) δ 9.35 (s, 1H), 7.62 (d, J=1.2 Hz, 1H), 7.52 (s, 1H), 7.44 (d, J=2.0 Hz, 1H), 7.35 (d, J=8.8 Hz, 1H), 7.17 (d, J=8.8 Hz, 1H), 6.95 (d, J=8.4 Hz, 1H), 5.74 (d, J=7.2 Hz, 1H), 5.07-5.02 (m, 2H), 4.98-4.91 (m, 1H), 3.30-3.28 (m, 4H), 2.32 (s, 3H), 1.90 (s, 3H), 1.51 (d, J=6.8 Hz, 3H), 1.41 (t, J=5.2 Hz, 4H), 0.97 (s, 6H). MS (ESI): mass calcd. For C₃₂H₃₄BClF₃N₃O₄ 589.23, m/z found 590.3 [M+H]⁺. HPLC: 98.41% (220 nm), 98.01% (254 nm). Chiral purity: 97.26% ee.

### Example 92

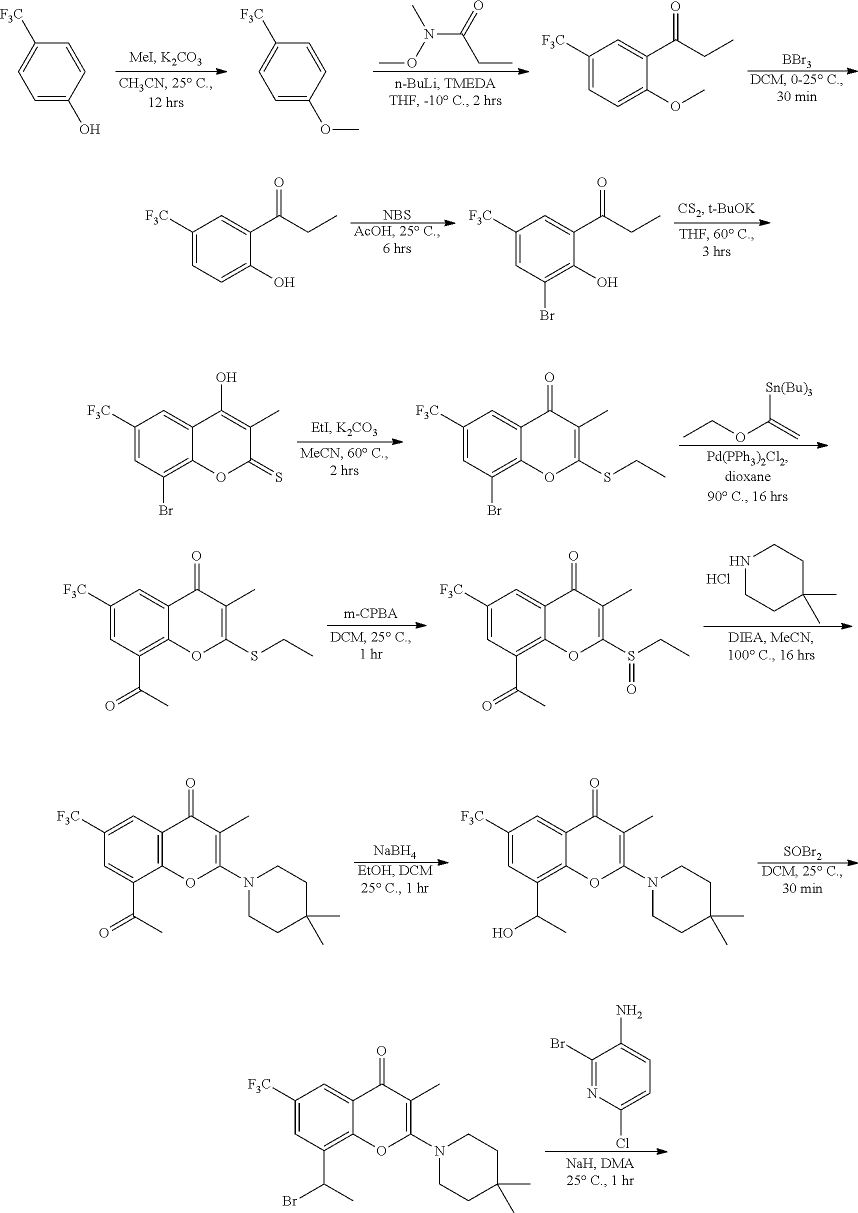

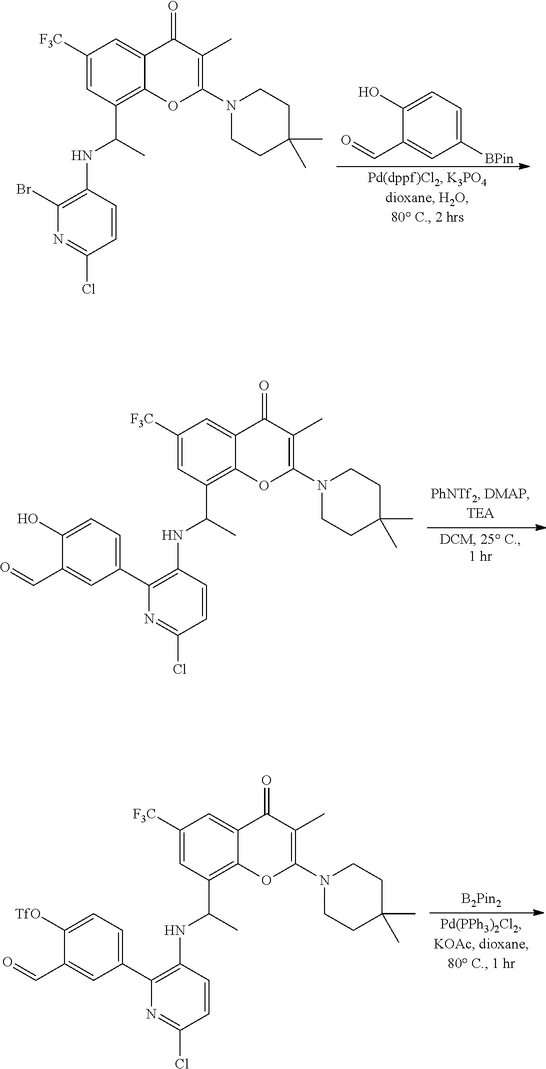

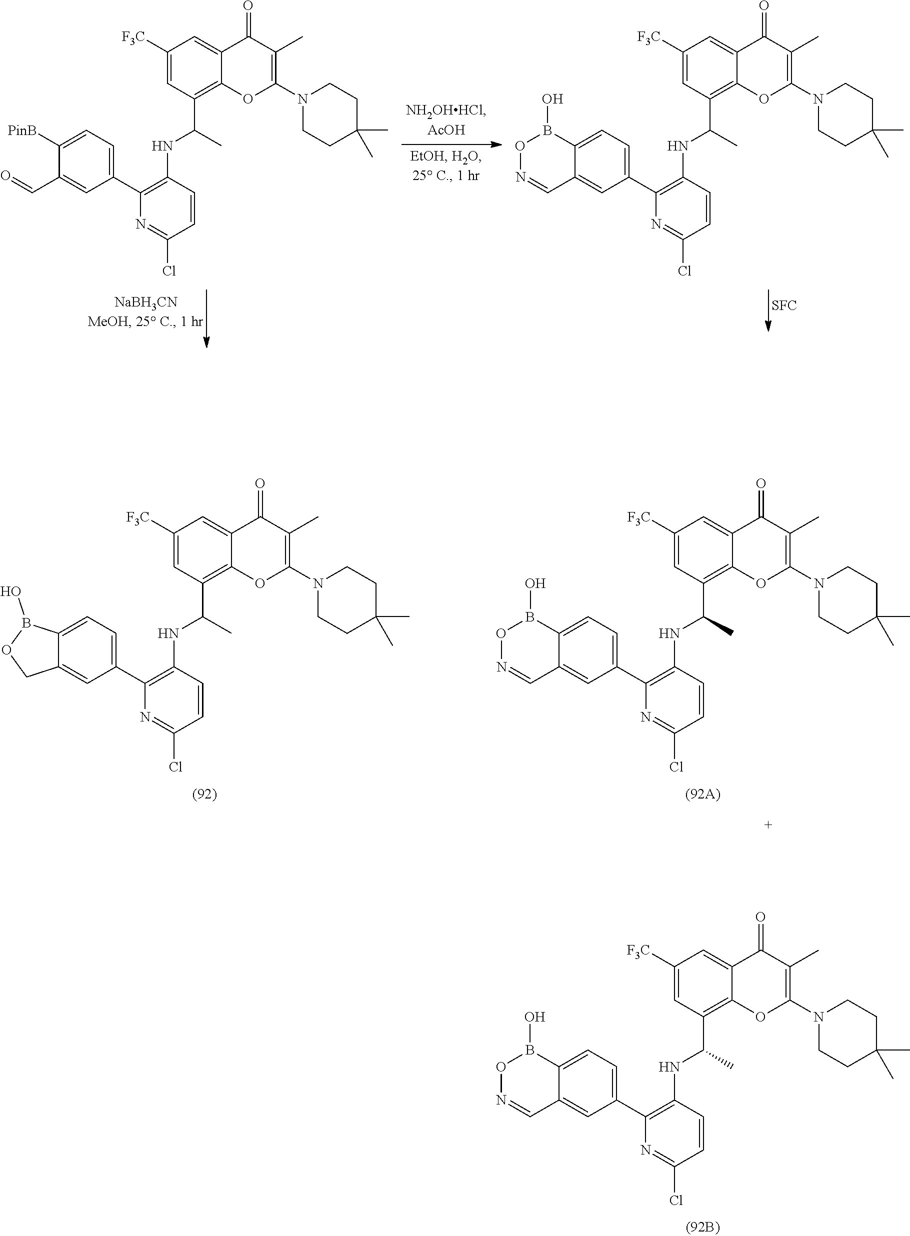

### Preparation of 1-methoxy-4-(trifluoromethyl)benzene

To a mixture of 4-(trifluoromethyl)phenol (100.00 g, 616.87 mmol, 1 eq) and K₂CO₃ (170.51 g, 1.23 mol, 2 eq) in MeCN (1 L) was added Mel (133.96 g, 943.81 mmol, 58.76 mL, 1.53 eq) dropwise at 25° C. under N₂, the reaction mixture was stirred at 25° C. for 12 h under N₂. The reaction mixture was filtrated and the filtrate was concentrated under reduced pressure to give a residue. The residue was poured into ice-water (300 mL) and extracted with MTBE (500 mL×3). The combined organic layers were washed with brine (500 mL), dried over Na₂SO₄, filtered and concentrated under reduced pressure to give the title compound (72.00 g, crude) as colorless oil, which was used directly for next step without further purification. ¹H NMR (CDCl₃, 400 MHz) δ 7.56 (d, J=8.8 Hz, 1H), 6.97 (d, J=8.8 Hz, 1H), 3.86 (s, 3H).

### Preparation of 1-[2-methoxy-5-(trifluoromethyl)phenyl]propan-1-one

To a solution of TMEDA (28.11 g, 241.86 mmol, 36.50 mL, 1.42 eq) in THF (50 mL) was added dropwise n-BuLi (2.5 M, 98.79 mL, 1.45 eq)(while keeping inner temperature between −10-10° C.) under N₂, the reaction mixture was stirred at −10° C. for 30 min. A solution of 1-methoxy-4-(trifluoromethyl)benzene (30.00 g, 170.32 mmol, 1 eq) in THF (200 mL) was added dropwise into the reaction mixture at −10° C. under N₂ and then the resulting reaction mixture was stirred at −10° C. for 1 h. Then N-methoxy-N-methyl-propanamide (29.93 g, 255.49 mmol, 1.5 eq) was added dropwise into the reaction mixture at −10° C. and stirred at −10° C. for 30 min. The 2 reactions were combined for work up. The reaction mixture was poured into ice-aq. NH₄Cl (500 mL) under N₂ at 25° C. slowly, and then extracted with EtOAc (500 mL×3). The combined organic layers were washed with brine (800 mL), dried over Na₂SO₄, filtered and concentrated under reduced pressure to give a residue. The residue was purified by flash silica gel chromatography (Biotage®; 330+330 g SepaFlash® Silica Flash Column, Eluent of 0~5% Ethyl acetate/Petroleum ether gradient @150 mL/min). The eluent was concentrated under reduced pressure to give the title compound (45.00 g, 193.80 mmol, 56.89% yield) as light yellow oil. ¹H NMR (CDCl₃, 400 MHz) δ 7.96 (d, J=2.4 Hz, 1H), 7.70 (dd, J=2.0, 8.4 Hz, 1H), 7.05 (d, J=8.8 Hz, 1H), 3.97 (s, 3H), 3.00 (q, J=7.2 Hz, 2H), 1.18 (t, J=7.2 Hz, 3H).

### Preparation of 1-[2-hydroxy-5-(trifluoronmethyl)phenyl]propan-1-one

To a solution of 1-[2-methoxy-5-(trifluoromethyl)phenyl]propan-1-one (45 g, 193.80 mmol, 1 eq) in DCM (400 mL) was added dropwise BBr₃ (2 M in DCM, 106.59 mL, 1.1 eq) at 0° C., the reaction mixture was warmed to 25° C. and stirred at 25° C. for 30 min. The reaction mixture was poured into ice-water (400 mL) under N₂ slowly, and then extracted with DCM (400 mL×3). The combined organic layers were washed with aq. sat. NaHCO₃ (600 mL), brine (500 mL), dried over Na₂SO₄, filtered and concentrated under reduced pressure to give a residue. The residue was purified by flash silica gel chromatography (Biotage®; 330 g SepaFlash® Silica Flash Column, Eluent of 0~2% Ethyl acetate/Petroleum ether gradient @150 mL/min). The eluent was concentrated under reduced pressure to give the title compound (23 g, 105.42 mmol, 54.40% yield) as colorless oil. ¹H NMR (CDCl₃, 400 MHz) δ 12.65 (s, 1H), 8.04 (d, J=1.2 Hz, 1H), 7.70 (dd, J=2.4, 8.8 Hz, 1H), 7.10 (d, J=8.8 Hz, 1H), 3.10 (q, J=7.2 Hz, 2H), 1.27 (t, J=7.2 Hz, 3H).

### Preparation of 1-[3-bromo-2-hydroxy-5-(trifluoromethyl)phenyl]propan-1-one

To a solution of 1-[2-hydroxy-5-(trifluoromethyl)phenyl]propan-1-one (22.00 g, 100.84 mmol, 1 eq) in AcOH (220 mL) was added NBS (21.54 g, 121.01 mmol, 1.2 eq) in portions at 10° C., the reaction mixture was warmed to 25° C. and stirred at 25° C. for 6 h. The reaction mixture was poured into ice-water (300 mL) under N₂ slowly, and then extracted with EtOAc (200 mL×3). The combined organic layers were washed with aq. sat. NaHCO₃ (600 mL×2), brine (500 mL), dried over Na₂SO₄, filtered and concentrated under reduced pressure to give a residue. The residue was purified by flash silica gel chromatography (Biotage®); 330 g SepaFlash® Silica Flash Column, Eluent of 0~10% Ethyl acetate/Petroleum ether gradient @150 mL/min). The eluent was concentrated under reduced pressure to give crude product. The crude product was triturated with Petroleum ether (50 mL) at 25° C. for 10 min, and then filtered. The filter cake was washed with Petroleum ether (5 mL×3), dried in vacuum to give the title compound (13.50 g, 45.44 mmol, 45.07% yield) as a white solid. ¹H NMR (CDCl₃, 400 MHz) δ 13.40 (s, 1H), 8.00-7.98 (m, 2H), 3.13 (q, J=7.6 Hz, 2H), 1.29 (t, J=7.2 Hz, 3H).

### Preparation of 8-bromo-4-hydroxy-3-methyl-6-(trifluoromethyl)chromene-2-thione

To a solution of 1-[3-bromo-2-hydroxy-5-(trifluoromethyl)phenyl]propan-1-one (13.50 g, 45.44 mmol, 1 eq) in THF (150 mL) was added t-BuOK (15.30 g, 136.33 mmol, 3 eq) in portions at 0° C., the reaction mixture was stirred at 0° C. for 10 min. Then CS₂ (5.19 g, 68.17 mmol, 4.11 mL, 1.5 eq) was added dropwise into the reaction mixture at 0° C. and the reaction mixture was stirred at 60° C. for 3 h. The reaction mixture was cooled to 25° C., poured into ice-water (150 mL), adjusted to pH=4 with HCl (2N) and extracted with EtOAc (150 mL×3). The combined organic layers were washed with brine (200 mL), dried over Na₂SO₄, filtered and concentrated under reduced pressure to give a crude product. The crude product was triturated with Petroleum ether/Ethyl acetate (v/v=10/1, 80 mL) at 25 C for 20 min, and then filtered. The filter cake was washed with Petroleum ether (10 mL×2), dried in vacuum to give the title compound (12.7 g, 37.45 mmol, 82.41% yield) as a yellow solid. ¹H NMR (CDCl₃, 400 MHz) δ 8.44 (s, 1H), 8.07 (s, 1H), 4.11 (s, 1H), 2.12 (s, 3H).

### Preparation of 8-bromo-2-ethylsulfanyl-3-methyl-6-(trifluoromethyl)chromen-4-one

To a solution of 8-bromo-4-hydroxy-3-methyl-6-(trifluoromethyl)chromene-2-thione (12.70 g, 37.45 mmol, 1 eq) in MeCN (150 mL) were added K2CO₃ (6.21 g, 44.94 mmol, 1.2 eq) and iodoethane (11.68 g, 74.90 mmol, 5.99 mL, 2 eq) in turns at 25° C., the reaction mixture was heated to 60° C. and stirred at 60° C. for 2 h. The reaction mixture was cooled to 25° C., poured into ice-water (150 mL) and extracted with EtOAc (150 mL×3). The combined organic layers were washed with brine (200 mL), dried over Na₂SO₄, filtered and concentrated under reduced pressure to give a residue. The residue was purified by flash silica gel chromatography (Biotage®; 120 g SepaFlash® Silica Flash Column, Eluent of 0~10% Ethyl acetate/Petroleum ether gradient @150 mL/min). The eluent was concentrated under reduced pressure to give the title compound (13.70 g, 37.31 mmol, 99.63% yield) as yellow solid. ¹H NMR (CDCl₃, 400 MHz) δ 8.46 (d, J=1.6 Hz, 1H), 8.05 (d, J=2.0 Hz, 1H), 3.35 (q, J=7.6 Hz, 2H), 2.08 (s, 3H), 1.52 (t, J=7.6 Hz, 3H).

### Preparation of 8-acetyl-2-ethylsulfanyl-3-methyl-6-(trifluoromethyl)chromen-4-one

A mixture of 8-bromo-2-ethylsulfanyl-3-methyl-6-(trifluoromethyl)chromen-4-one (13.70 g, 37.31 mmol, 1 eq), tributyl(1-ethoxyvinyl)stannane (16.89 g, 46.77 mmol, 15.80 mL, 1.25 eq) and Pd(PPh₃)₂Cl₂ (2.62 g, 3.73 mmol, 0.1 eq) in dioxane (150 mL) was degassed and purged with N₂ for 3 times at 25° C., and then the reaction mixture was heated to 90° C. and stirred at 90° C. for 16 h under N₂ atmosphere. The reaction mixture was cooled to 25° C., and then HCl (130 mL, 2 M) was added to the mixture and stirred at 50° C. for 0.5 h. The reaction mixture was cooled to 25° C. and added sat. KF (130 mL) and stirred for 0.5 h. The resulting suspension was directly filtered, the filter cake was washed with EtOAc (50 mL×3) and the filtrate was extracted with EtOAc (100 mL×3). The combined organic phases were washed with brine (200 mL), dried over Na₂SO₄, filtered and concentrated under reduced pressure. The residue was purified by flash silica gel chromatography (Biotage®; 120 g SepaFlash® Silica Flash Column, Eluent of 0~15% Ethyl acetate/Petroleum ether gradient @120 mL/min). The eluent was concentrated under reduced pressure to give the title compound (10.40 g, 31.48 mmol, 84.38% yield) as a white solid. ¹H NMR (CDCl₃, 400 MHz) δ 8.68 (d, J=1.6 Hz, 1H), 8.18 (d, J=2.0 Hz, 1H), 3.32 (q, J=7.2 Hz, 2H), 2.76 (s, 3H), 2.11 (s, 3H), 1.45 (t, J=7.6 Hz, 3H).

### Preparation of 8-acetyl-2-ethylsulfinyl-3-methyl-6-(trifluoromethyl)chromen-4-one

To a solution of 8-acetyl-2-ethylsulfanyl-3-methyl-6-(trifluoromethyl)chromen-4-one (10.40 g, 31.48 mmol, 1 eq) in DCM (120 mL) was added m-CPBA (7.67 g, 37.78 mmol, 1.2 eq) at 0° C. in portions. The reaction mixture was warmed to 25° C. and stirred at 25° C. for 1 h. The reaction mixture was poured into sat. aq.Na₂SO₃ (100 mL) and then extracted with DCM (100 mL×3). The combined organic layers were washed with brine (200 mL), dried over Na₂SO₄, filtered and concentrated under reduced pressure to give a residue. The crude product was triturated with Petroleum ether/Ethyl acetate (v/v=5:1, 50 mL) at 25 C for 10 min, and then filtered. The filter cake was washed with Petroleum ether (5 mL×2), dried in vacuum to give the title compound (9 g, 25.99 mmol, 82.54% yield) as an off-white solid. ¹H NMR (CDCl₃, 400 MHz) δ 8.67 (d, J=2.0 Hz, 1H), 8.42 (d, J=2.4 Hz, 1H), 3.27-3.18 (m, 2H), 2.94 (s, 3H), 2.24 (s, 3H), 1.41 (t, J=7.2 Hz, 3H).

### Preparation of 8-acetyl-2-(4,4-dimethyl-1-piperidyl)-3-methyl-6-(trifluoromethyl) chromen-4-one

To a solution of 8-acetyl-2-ethylsulfinyl-3-methyl-6-(trifluoromethyl)chromen-4-one (3.00 g, 8.66 mmol, 1 eq) and 4,4-dimethylpiperidine (2.59 g, 17.32 mmol, 2eq, HCl) in MeCN (30 mL) was added DIEA (5.60 g, 43.31 mmol, 7.54 mL, 5 eq) in one portion at 25° C., the reaction mixture was heated to 100° C. and stirred at 100° C. for 16 h. The reaction mixture was cooled to 25° C., poured into ice-water (30 mL) and then extracted with EtOAc (30 mL×3). The combined organic layers were washed with brine (50 mL), dried over Na₂SO₄, filtered and concentrated under reduced pressure to give a residue. The residue was purified by flash silica gel chromatography (Biotage®; 40 g SepaFlash® Silica Flash Column, Eluent of 0~15% Ethyl acetate/Petroleum ether gradient @80 mL/min). The eluent was concentrated under reduced pressure to give the title compound (2.80 g, 7.34 mmol, 84.75% yield) as a yellow solid. ¹H NMR (CDCl₃, 400 MHz) δ 8.66 (d, J=1.6 Hz, 1H), 8.17 (d, J=2.0 Hz, 1H), 3.51-3.49 (m, 4H), 2.76 (s, 3H), 2.08 (s, 3H), 1.56-1.53 (m, 4H), 1.06 (s, 6H).

### Preparation of 2-(4,4-dimethyl-1-piperidyl)-8-(1-hydroxyethyl)-3-methyl-6-(trifluoro methyl)chromen-4-one

To a solution of 8-acetyl-2-(4,4-dimethyl-1-piperidyl)-3-methyl-6-(trifluoromethyl)chromen-4-one (2.80 g, 7.34 mmol, 1 eq) in EtOH (20 mL)/DCM (20 mL) was added NaBH₄ (690 mg, 18.24 mmol, 2.48 eq) in portions at 0° C. under N₂, the mixture was warmed to 25° C. stirred at 25° C. for 1 h. The reaction mixture was cooled to 0° C., diluted with H₂O (30 mL), adjusted to pH=6 with HCl (3 M) and then extracted with DCM (30 mL×3). The combined organic phase was washed with brine (50 mL), dried over Na₂SO₄, filtered and concentrated under reduced pressure to give the title compound (3.00 g, crude) as a light yellow solid, which was used directly for next step without further purification. ¹H NMR (CDCl₃, 400 MHz) δ 8.19 (d, J=1.2 Hz, 1H), 7.87 (d, J=2.0 Hz, 1H), 5.35 (q, J=6.4 Hz, 1H), 3.46-3.43 (m, 4H), 2.10 (s, 3H), 1.57-1.51 (m, 7H), 1.06 (s, 6H).

### Preparation of 8-(1-bromoethyl)-2-(4,4-dimethyl-1-piperidyl)-3-methyl-6-(trifluoro methyl)chromen-4-one

To a solution of 2-(4,4-dimethyl-1-piperidyl)-8-(1-hydroxyethyl)-3-methyl-6-(trifluoromethyl) chromen-4-one (3.00 g, 7.82 mmol, 1 eq) in DCM (30 mL)/DMF (12.04 L) was added SOBr₂ (2.11 g, 10.17 mmol, 788.11 μL, 1.3 eq) dropwise at 0° C., the reaction mixture was warmed to 25° C. and stirred for 20 min at 25° C. The reaction mixture was poured into ice-H₂O (50 mL) and then extracted with DCM (30 mL×3). The combined organic layers were washed with sat.aq NaHCO₃ until pH=8, then washed with brine (60 mL), dried over Na₂SO₄, filtered and concentrated under reduced pressure to give the title compound (4.00 g, crude) as a brown solid, which was used directly for next step without further purification. ¹H NMR (CDCl₃, 400 MHz) δ 8.46 (s, 1H), 7.90 (s, 1H), 5.64 (q, J=7.2 Hz, 1H), 3.54-3.51 (m, 4H), 2.19 (d, J=7.2 Hz, 3H), 2.06 (s, 3H), 1.58-1.56 (m, 4H), 1.08 (s, 6H).

### Preparation of 8-[1-[(2-bromo-6-chloro-3-pyridyl)amino]ethyl]-2-(4,4-dimethyl-1-piperidyl)-3-methyl-6-(trifluoromethyl)chromen-4-one

The title compound was prepared as an orange yellow solid (2.30 g, 4.02 mmol, 44.80% yield) according to the procedures described herein and the synthetic scheme, using 2-bromo-6-chloro-pyridin-3-amine (1.67 g, 8.07 mmol, 0.9 eq) and 8-(1-bromoethyl)-2-(4,4-dimethyl-1-piperidyl-3-methyl-6-(trifluoromethyl)chromen-4-one (4.00 g, 8.96 mmol, 1 eq). ¹H NMR (CDCl₃, 400 MHz) δ 8.42 (d, J=1.6 Hz, 1H), 7.72 (d, J=2.0 Hz, 1H), 6.99 (d, J=8.4 Hz, 1H), 6.45 (d, J=8.4 Hz, 1H), 4.94 (q, J=7.2 Hz, 1H), 4.82 (d, J=4.4 Hz, 1H), 3.46-3.44 (m, 4H), 2.07 (s, 3H), 1.74 (d, J=6.4 Hz, 3H), 1.56-1.53 (m, 4H), 1.06 (s, 6H).

### Preparation of 5-[6-chloro-3-[1-[2-(4,4-dimethyl-1-piperidyl)-3-methyl-4-oxo-6-(trifluoromethyl)chromen-8-yl]ethylamino]-2-pyridyl]-2-hydroxy-benzaldehyde

The title compound was prepared as a yellow solid (1.90 g, 3.09 mmol, 84.40% yield) according to the procedures described herein and the synthetic scheme, using 8-[1-[(2-bromo-6-chloro-3-pyridyl)amino]ethyl]-2-(4,4-dimethyl-1-piperidyl)-3-methyl-6-(trifluoromethyl)chromen-4-one (2.10 g, 3.67 mmol, 1 eq) and 2-hydroxy-5-(4,4,5,5-tetramethyl-1,3,2-dioxaborolan-2-yl)benzaldehyde (909 mg, 3.67 mmol, 1 eq). ¹H NMR (DMSO-d₆, 400 MHz) δ 11.17 (s, 1H), 9.98 (s, 1H), 8.40 (d, J=1.6 Hz, 1H), 7.95 (d, J=4.0 Hz, 1H), 7.89 (dd, J=2.4, 8.4 Hz, 1H), 7.70 (d, J=2.0 Hz, 1H), 7.15 (d, J=8.8 Hz, 1H), 7.02 (d, J=8.4 Hz, 1H), 6.61 (d, J=8.8 Hz, 1H), 4.94 (q, J=6.8 Hz, 1H), 4.44 (s, 1H), 3.43-3.40 (m, 4H), 2.06 (s, 3H), 1.60 (d, J=6.8 Hz, 3H), 1.54-1.51 (m, 4H), 1.05 (s, 6H).

### Preparation of [4-[6-chloro-3-[1-[2-(4,4-dimethyl-1-piperidyl)-3-methyl-4-oxo-6-(trifluoromethyl)chromen-8-yl]ethylamino]-2-pyridyl]-2-formyl-phenyl]trifluoromethanesulfonate

The title compound was prepared as a yellow solid (1.20 g, 1.61 mmol, 51.98% yield) according to the procedures described herein and the synthetic scheme, using 5-[6-chloro-3-[1-[2-(4,4-dimethyl-1-piperidyl)-3-methyl-4-oxo-6-(trifluoromethyl)chromen-8-yl]ethylamino]-2-pyridyl]-2-hydroxy-benzaldehyde (1.90 g, 3.09 mmol, 1 eq) and 1,1,1-trifluoro-N-phenyl-N-(trifluoromethylsulfonyl)methanesulfonamide (2.21 g, 6.19 mmol, 2 eq). ¹H NMR (DMSO-d₆, 400 MHz) δ 10.34 (s, 1H), 8.41 (d, J=1.6 Hz, 1H), 8.40 (d, J=2.4 Hz, 1H), 8.12 (dd, J=2.4, 8.4 Hz, 1H), 7.70 (d, J=2.4 Hz, 1H), 7.60 (d, J=8.4 Hz, 1H), 7.07 (d, J=8.8 Hz, 1H), 6.64 (d, J=8.8 Hz, 1H), 4.98 (q, J=6.4 Hz, 1H), 4.41 (s, 1H), 3.49-3.42 (m, 4H), 2.07 (s, 3H), 1.63 (d, J=6.4 Hz, 3H), 1.56-1.53 (m, 4H), 1.06 (s, 6H).

### Preparation of 5-[6-chloro-3-[1-[2-(4,4-dimethyl-1-piperidyl)-3-methyl-4-oxo-6-(trifluoromethyl)chromen-8-yl]ethylamino]-2-pyridyl]-2-(4,4,5,5-tetramethyl-1,3,2-dioxaborolan-2-yl)benzaldehyde

The title compound was prepared as a brown solid (2.00 g, crude) according to the procedures described herein and the synthetic scheme, using [4-[6-chloro-3-[1-[2-(4,4-dimethyl-1-piperidyl)-3-methyl-4-oxo-6-(trifluoromethyl)chromen-8-yl]ethylamino]-2-pyridyl]-2-formyl-phenyl]trifluoromethanesulfonate (1.20 g, 1.61 mmol, 1 eq), and B₂Pin₂ (1.23 g, 4.82 mmol, 3 eq). The product was used directly for next step without further purification.

### Preparation of 8-[1-[[6-chloro-2-(1-hydroxy-2,3,1-benzoxazaborinin-6-yl)-3-pyridyl]amino]ethyl]-2-(4,4-dimethyl-1-piperidyl)-3-methyl-6-(trifluoromethyl)chromen-4-one

The title compound was prepared as a white solid (0.1 g, 144.68 μmol, 6.98% yield) according to the procedures described herein and the synthetic scheme, using 5-[6-chloro-3-[1-[2-(4,4-dimethyl-1-piperidyl)-3-methyl-4-oxo-6-(trifluoromethyl)chromen-8-yl]ethylamino]-2-pyridyl]-2-(4,4,5,5-tetramethyl-1,3,2-dioxaborolan-2-yl)benzaldehyde (1.50 g, 2.07 mmol, 1 eq) and hydroxylamine;hydrochloride (216 mg, 3.11 mmol, 1.5 eq). ¹H NMR (DMSO-d₆, 400 MHz) δ 9.41 (br s, 1H), 8.66 (s, 1H), 8.13 (d, J=7.6 Hz, 1H), 8.07 (s, 1H), 8.07-8.01 (m, 2H), 7.91 (d, J=2.0 Hz, 1H), 7.19 (d, J=7.2 Hz, 1H), 7.06 (d, J=8.4 Hz, 1H), 6.10 (d, J=8.0 Hz, 1H), 4.96 (q, J=7.2 Hz, 1H), 3.38-3.33 (m, 4H), 1.93 (s, 3H), 1.54 (d, J=6.8 Hz, 3H), 1.44-1.41 (m, 4H), 0.98 (s, 6H).

### Preparation of 8-[1-[[6-chloro-2-(1-hydroxy-2,3,1-benzoxazaborinin-6-yl)-3-pyridyl]amino]ethyl]-2-(4,4-dimethyl-1-piperidyl)-3-methyl-6-(trifluoromethyl)chromen-4-one

8-[1-[[6-chloro-2-(1-hydroxy-2,3,1-benzoxazaborinin-6-yl)-3-pyridyl]amino]ethyl]-2-(4,4-dimethyl-1-piperidyl)-3-methyl-6-(trifluoromethyl)chromen-4-one (0.1 g. 156.53 μmol, 1 eq) was separated by SFC (column: DAICEL CHIRALPAK IG (250 mm×30 mm×10 um); mobile phase: [CO₂-IPA]; B %:50%, isocratic elution mode) to give the title compound, Isomer 1 (4 mg, 6.26 gmol, 4.00% yield) as a white solid and the title compound, Isomer 2 (12 mg, 18.78 μmol, 12.00% yield) as a white solid. Isomer 1: ¹H NMR (DMSO-d₆, 400 MHz) δ 8.63 (s, 1H), 8.11 (d, J=7.6 Hz, 1H), 8.06 (d, J=2 Hz, 1H), 8.02-7.99 (s, 1H), 7.90 (d, J=2 Hz, 2H), 7.18 (d, J=8.8 Hz, 1H), 7.05 (d, J=8.8 Hz, 1H), 6.08 (d, J=7.6 Hz, 1H), 4.99-4.94 (m, 1H), 3.36 (s, 4H), 1.92 (s, 3H), 1.53 (d, J=6.8 Hz, 3H), 1.43-1.40 (m, 4H), 0.97 (s, 6H). MS (ESI): mass calcd. For C₃₂H₃₁BClF₃N₄O₄ 638.21, m/z found 639.1 [M+H]⁺. HPLC: 91.29% (220 nm), 91.27% (254 nm). Chiral purity: 100% ee. Isomer 2: ¹H NMR (DMSO-d₆, 400 MHz) δ 9.46 (s, 1H), 8.67 (s, 1H), 8.13 (d, J=7.6 Hz, 1H), 8.06 (d, J 1.6 Hz 1H), 8.04-8.02 (m, 2H), 7.91 (d, J=2.0 Hz, 1H), 7.19 (d, J=8.8 Hz, 1H), 7.05 (d, J=8.8 Hz, 1H), 6.11 (m, J=7.6 Hz, 1H), 3.37-3.35 (m, 4H), 1.92 (s, 3H), 1.53 (d, J=6.8 Hz, 3H), 1.43-1.41 (m, 4H), 0.97 (s, 6H). MS (ESI): mass calcd. For C₃₂H₃₁BClF₃N₄O₄ 638.21, m/z found 639.1 [M+H]⁺. HPLC: 99.00% (220 nm), 99.74% (254 nm). Chiral purity: 100% ee

### Preparation of 8-[1-[[6-chloro-2-(1-hydroxy-3H-2,1-benzoxaborol-5-yl)-3-pyridyl]amino]ethyl]-2-(4,4-dimethyl-1-piperidyl)-3-methyl-6-(trifluoromethyl)chromen-4-one

To a solution of 5-[6-chloro-3-[1-[2-(4,4-dimethyl-1-piperidyl)-3-methyl-4-oxo-6-(trifluoromethyl)chromen-8-yl]ethylamino]-2-pyridyl]-2-(4,4,5,5-tetramethyl-1,3,2-dioxaborolan-2-yl)benzaldehyde (0.45 g, 621.53 μmol, 1 eq) in MeOH (5 mL) was added NaBH₃CN (58.59 mg, 932.30 mol, 1.5 eq) at 0° C. The reaction mixture was warmed to 25° C. and stirred at 25° C. for 1 hr. LC-MS showed 5-[6-chloro-3-[1-[2-(4,4-dimethyl-1-piperidyl)-3-methyl-4-oxo-6-(trifluoromethyl)chromen-8-yl]ethylamino]-2-pyridyl]-2-(4,4,5,5-tetramethyl-1,3,2-dioxaborolan-2-yl)benzaldehyde was consumed completely and one main peak with desired MS was detected. To the reaction mixture was added dropwise H₂O (5 mL) at 25° C. The resulting precipitate was collected by filtration. The filter cake was concentrated under reduced pressure to give the crude product. The residue was purified by prep-HPLC (column: Waters Xbridge BEH C18 100×30 mm×10 um; mobile phase: [H₂O (10 mM NH₄HCO₃)-AC□]; gradient: 50%-90% B over 8.0 min). The eluent was lyophilized to give the product to give the title compound (32.9 mg, 52.26 μmol, 8.41% yield) as a white solid. ¹H NMR (DMSO-d₆, 400 MHz) δ 9.27 (s, 1H), 8.08 (d, J=1.6 Hz, 1H), 7.90 (d, J=2.4 Hz, 1H), 7.82 (d, J=7.6 Hz, 1H), 7.65-7.62 (m, 2H), 7.14 (d, J=8.4 Hz, 1H), 7.02 (d, J=8.8 Hz, 1H), 5.83 (d, J=7.6 Hz, 1H), 5.04-4.94 (m, 3H), 3.37-3.36 (m, 4H), 1.94 (s, 3H), 1.54 (d, J=6.8 Hz, 3H), 1.44-1.41 (m, 4H), 0.98 (s, 6H). MS (ESI): mass calcd. For C₃₂H₃₂BClF₃N₃O₄ 625.21, m/z found 626.3 [M+H]⁺. HPLC: 99.41% (220 nm), 99.37% (254 nm).

### Example 93

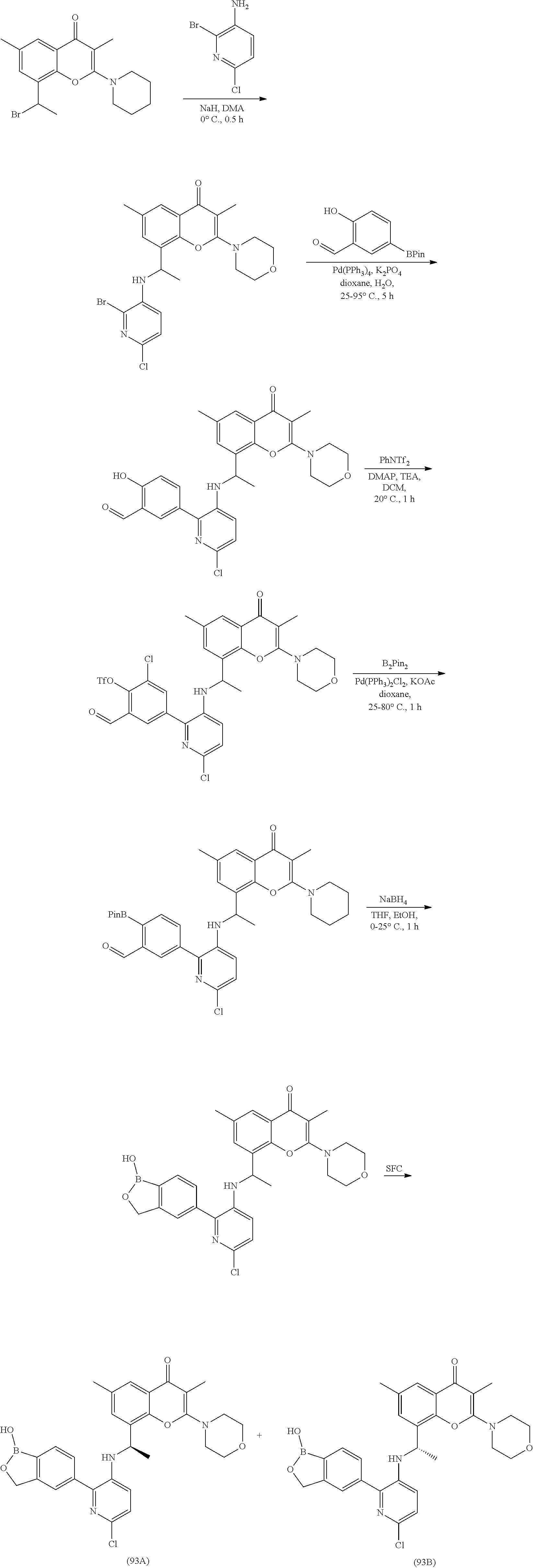

### Preparation of 8-(1-((2-bromo-6-chloropyridin-3-yl)amino)ethyl)-3,6-dimethyl-2-morpholino-4H-chromen-4-one

To a solution of 2-bromo-6-chloro-pyridin-3-amine (623 mg, 3.00 mmol, 1.1 eq) in DMA (10 mL) was added NaH (163 mg, 4.10 mmol, 1.5 eq) in one portion at 0° C., the reaction mixture was stirred at 0° C. for 10 min. Then a solution of 8-(1-bromoethyl)-3,6-dimethyl-2-morpholino-chromen-4-one (1.00 g, 2.73 mmol, 1 eq) in DMA (10 mL) was added dropwise to the mixture above, the reaction mixture was stirred at 0° C. for 20 min. The reaction mixture was quenched by H₂O (30 mL) at 0° C., and then extracted with EtOAc (20 mL×3). The combined organic layers were washed with brine (20 mL×3), dried over Na₂SO₄, filtered and concentrated under reduced pressure to give a residue. The residue was purified by flash silica gel chromatography (Biotage®; 40 g SepaFlash® Silica Flash Column, Eluent of 0~100% Ethyl acetate/Petroleum ether gradient @60 m/min) to give the title compound (550 mg, 1.12 mmol, 40.88% yield) as a brown solid. ¹H NMR (DMSO-d₆, 400 MHz) δ 7.65 (s, 1H), 7.42 (s, 1H), 7.18 (d, J=8.4 Hz, 1H), 6.80 (d, J=8.4 Hz, 1H), 5.80 (d, J=6.8 Hz, 1H), 5.03 (t, J=6.8 Hz, 1H), 3.75-3.73 (m, 4H), 3.44-3.42 (m, 4H), 2.32 (s, 3H), 1.93 (s, 1H), 1.63 (d, J=6.4 Hz, 3H).

### Preparation of 5-(6-chloro-3-((1-(3,6-dimethyl-2-morpholino-4-oxo-4H-chromen-8-yl) ethyl)amino)pyridin-2-yl)-2-hydroxybenzaldehyde

To a solution of 8-[1-[(2-bromo-6-chloro-3-pyridyl)amino]ethyl]-3,6-dimethyl-2-morpholino-chromen-4-one (500 mg, 1.01 mmol, 1 eq) in dioxane (15 mL)/H₂O (5 mL) were added 2-hydroxy-5-(4,4,5,5-tetramethyl-1,3,2-dioxaborolan-2-yl)benzaldehyde (302 mg, 1.22 mmol, 1.2 eq), K₃PO₄ (431 mg, 2.03 mmol, 2 eq) and Pd(PPh₃)₄ (235 mg, 202.93 μmol, 0.2 eq) in turns at 25° C., the reaction mixture was heated to 95° C. and stirred at 95° C. for 5 h. The reaction mixture was cooled to 25° C., diluted with H₂O (20 mL), and then extracted with EtOAc (10 mL×3). The combined organic layers were washed with brine (10 mL×3), dried over Na₂SO₄, filtered and concentrated under reduced pressure to give a residue. The residue was purified by flash silica gel chromatography (Biotage®; 20 g SepaFlash® Silica Flash Column, Eluent of 0~50% Ethyl acetate/Petroleum ether gradient @40 mL/min) to give the title compound (600 mg, 77% yield) as yellow oil. ¹H NMR (DMSO-d₆, 400 MHz) δ 11.02 (s, 1H), 10.33 (s, 1H), 8.04 (s, 1H), 7.92-7.90 (m, 1H), 7.59 (s, 1H), 7.56 (s, 1H), 7.14-7.10 (m, 2H), 7.08 (d, J=6.8 Hz, 1H), 5.64 (d, J=6.8 Hz, 1H), 4.94 (t, J=7.2 Hz, 1H), 3.74-3.69 (m, 4H), 3.43-3.35 (m, 4H), 2.32 (s, 3H), 1.93 (s, 3H), 1.50 (d, J=6.8 Hz, 3H).

### Preparation of 4-(6-chloro-3-((1-(3,6-dimethyl-2-morpholino-4-oxo-4H-chromen-8-yl) ethyl)amino)pyridin-2-yl)-2-formylphenyl trifluoromethanesulfonate

A mixture of 5-[6-chloro-3-[1-(3,6-dimethyl-2-morpholino-4-oxo-chromen-8-yl)ethylamino]-2-pyridyl]-2-hydroxy-benzaldehyde (600 mg, 1.12 mmol, 1 eq), 1,1,1-trifluoro-N-phenyl-N-(trifluoromethylsulfonyl)methanesulfonamide (803 mg, 2.25 mmol, 2 eq), DMAP (14 mg, 112 μmol, 0.1 eq) and TEA (227 mg, 2.25 mmol, 313 μL, 2 eq) in DCM (6 mL) was degassed and purged with N₂ for 3 times at 20° C., and then the reaction mixture was stirred at 20° C. for 1 h under N₂ atmosphere. The reaction mixture was concentrated under reduced pressure to remove the solvent and the residue was purified by flash silica gel chromatography (Biotage®; 40 g SepaFlash® Silica Flash Column, Eluent of 0~50% Ethyl acetate/Petroleum ether gradient @60 mL/min) to give the title compound (700 mg, 1.05 mmol, 74.8% yield) as a yellow solid. ¹H NMR (DMSO-d₆, 400 MHz) δ 10.16 (s, 1H), 8.48 (s, 1H), 8.29-8.26 (m, 1H), 7.75 (d, J=6.8 Hz, 1H), 7.49 (s, 1H), 7.47 (s, 1H), 7.19 (d, J=8.8 Hz, 1H), 6.93 (d, J=8.8 Hz, 1H), 6.16 (d, J=6.8 Hz, 1H), 4.97 (t, J=6.8 Hz, 1H), 3.74-3.72 (m, 4H), 3.43-3.39 (m, 4H), 2.32 (s, 3H), 1.93 (s, 3H), 1.52 (d, J=6.4 Hz, 3H).

### Preparation of 5-(6-chloro-3-((1-(3,6-dimethyl-2-morpholino-4-oxo-4H-chromen-8-yl) ethyl)amino)pyridin-2-yl)-2-(4,4,5,5-tetramethyl-1,3,2-dioxaborolan-2-yl)benzaldehyde

A mixture of [4-[6-chloro-3-[1-(3,6-dimethyl-2-morpholino-4-oxo-chromen-8-yl)ethylamino]-2-pyridyl]-2-formyl-phenyl]trifluoromethanesulfonate (700 mg, 1.05 mmol, 1 eq), 4,4,5,5-tetramethyl-2-(4,4,5,5-tetramethyl-1,3,2-dioxaborolan-2-yl)-1,3,2-dioxaborolane (1.33 g, 5.25 mmol, 5 eq), KOAc (206 mg, 2.10 mmol, 2 eq) and Pd(PPh₃)₂Cl₂ (74 mg, 105 μmol, 0.1 eq) in dioxane (15 mL) was degassed and purged with N₂ for 3 times at 25° C., and then the reaction mixture was heated to 80° C. and stirred at 80° C. for 1 h under N₂ atmosphere. The reaction mixture was cooled to 25° C., quenched by H₂O (10 mL) and then extracted with EtOAc (15 mL×3). The combined organic layers were washed with brine, dried over Na₂SO₄, filtered and concentrated under reduced pressure to give a residue. The residue was purified by flash silica gel chromatography (Biotage®; 12 g SepaFlash® Silica Flash Column, Eluent of 0~60% Ethyl acetate/Petroleum ether gradient @40 nL/min) to give the title compound (470 mg, crude) as brown oil, which was used directly for next step without further purification.

### Preparation of 8-[(1S)-1-[[6-chloro-2-(1-hydroxy-3H-2,1-benzoxaborol-5-yl)-3-pyridyl]amino]ethyl]-3,6-dimethyl-2-morpholino-chromen-4-one and 8-[(1R)-1-[[6-chloro-2-(1-hy droxy-3H-2,1-benzoxaborol-5-yl)-3-pyridyl]amino]ethyl]-3,6-dimethyl-2-morpholino-chromen-4-one

To a solution of 5-[6-chloro-3-[1-(3,6-dimethyl-2-morpholino-4-oxo-chromen-8-yl)ethylamino]-2-pyridyl]-2-(4,4,5,5-tetramethyl-1,3,2-dioxaborolan-2-yl)benzaldehyde (470 mg, 730 mol, 1 eq) in THF (4 mL)/EtOH (2 mL) was added NaBH₄ (138 mg, 3.65 mmol, 5 eq) portion-wise at 0° C., the reaction mixture was warmed up to 25° C. and stirred at 25° C. for 1 h. The reaction mixture was poured into ice water (10 mL) at 0° C. and extracted with DCM (5 ml×3). The combined organic phase was washed with brine, dried over Na₂SO₄, filtered and concentrated under reduced pressure to give a residue. The residue was purified by flash silica gel chromatography (Biotage®; 12 g SepaFlash® Silica Flash Column, Eluent of 0~100% Ethyl acetate: Methanol @40 m/min) to give the racemate (180 mg, 45.18% yield). The racemate was further purified by SFC (column: DAICEL CHIRALCEL OD (250 mm×30 mm×10 um); mobile phase: [CO₂-EtOH]; B %:45%, isocratic elution mode) to give the title compound, Isomer 1 (47.2 mg, 86.48 μmol, 11.85% yield) as a white solid and the title compound, Isomer 2 (29.1 mg, 53.31 μmol, 7.30% yield) as a white solid. Isomer 1: ¹H NMR (DMSO-d₆, 400 MHz) δ 9.28 (s, 1H), 7.85 (d, J=8.0 Hz, 1H), 7.68-7.63 (m, 3H), 7.46 (d, J=2.0 Hz, 1H), 7.15 (d, J=8.8 Hz, 1H), 6.95 (d, J=8.8 Hz, 1H), 5.52 (d, J=7.6 Hz, 1H), 5.04 (d, J=3.2 Hz, 2H), 4.93 (t, J=6.8 Hz, 1H), 3.68 (t, J=4.4 Hz, 4H), 3.28-3.26 (m, 4H), 2.32 (s, 3H), 1.91 (s, 3H), 1.50 (d, J=6.8 Hz, 3H). MS (ESI): mass calcd. For C₂₉H₂₉BClN₃O₅ 545.83, m/z found 546.1 [M+H]⁺. HPLC: 98.34% (220 nm), 98.35% (254 nm). Chiral purity: 100 ee %. Isomer 2: ¹H NMR (DMSO-d₆, 400 MHz) δ 9.28 (s, 1H), 7.85 (d, J=8.0 Hz, 1H), 7.68-7.63 (m, 3H), 7.46 (d, J=2.0 Hz, 1H), 7.14 (d, J=8.8 Hz, 1H), 6.95 (d, J=8.8 Hz, 1H), 5.52 (d, J=7.6 Hz, 1H), 5.04 (d, J=3.2 Hz, 2H), 4.93 (t, J=6.8 Hz, 1H), 3.68 (t, J=4.4 Hz, 4H), 3.29-3.25 (m, 4H), 2.32 (s, 3H), 1.91 (s, 3H), 1.50 (d, J=6.8 Hz, 3H). MS (ESI): mass calcd. For C₂₉H₂₉BClN₃O₅ 545.83, m/z found 546.1 [M+H]⁺. HPLC: 97.71% (220 nm), 99.42% (254 nm). Chiral purity: 99.6 ee %

### Example 94

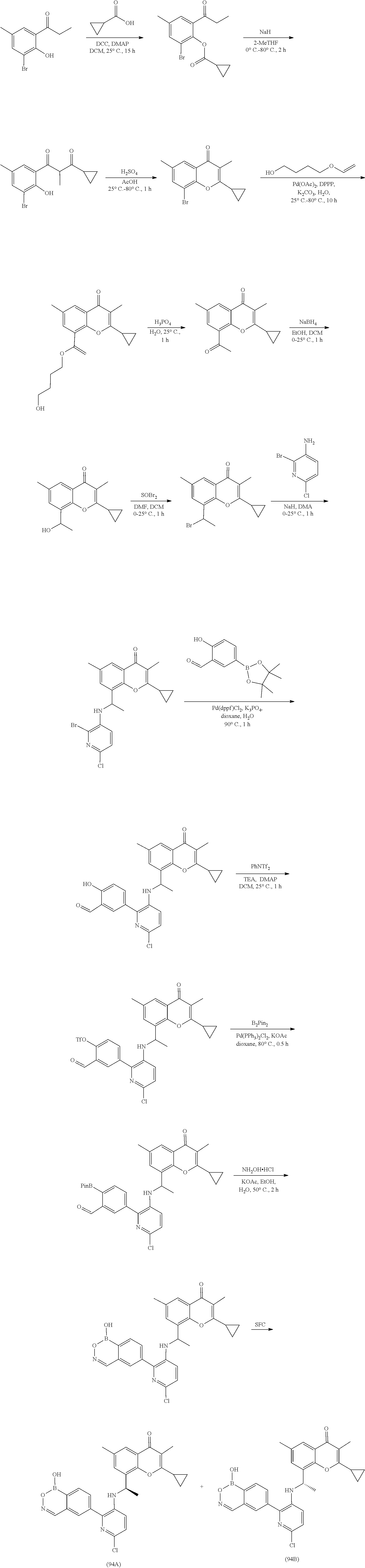

### Preparation of (2-bromo-4-methyl-6-propanoyl-phenyl) cyclopropanecarboxylate

To a mixture of 1-(3-bromo-2-hydroxy-5-methyl-phenyl)propan-1-one (20.00 g, 82.3 mmol, 1 eq), cyclopropanecarboxylic acid (8.50 g, 98.7 mmol, 7.81 mL, 1.2 eq) and DMAP (3.02 g, 24.7 mmol, 0.3 eq) in DCM (200 mL) was added dropwise a solution of DCC (27.2 g, 132 mmol, 26.6 mL, 1.6 eq) in DCM (100 mL) at 25° C., the reaction mixture was stirred at 25° C. for 15 h. The reaction mixture was quenched by H₂O (200 mL) and extracted with DCM (100 mL×3). The combined organic layers were washed with brine (200 mL×2), dried over Na₂SO₄, filtered and concentrated under reduced pressure to give a residue. The residue was purified by flash silica gel chromatography (Biotage®; 330 g SepaFlash® Silica Flash Column, Eluent of 0~5% Ethyl acetate/Petroleum ether gradient @80 mL/min) to give the title compound (10.0 g, 32.14 mmol, 39.06% yield) as a yellow gum. ¹H NMR (CDCl₃, 400 MHz) δ 7.55 (s, 1H), 7.45 (s, 1H), 2.86 (q, J=7.2 Hz, 2H), 2.37 (s, 3H), 1.96-1.88 (m, 1H), 1.61 (s, 3H), 1.28-1.25 (m, 2H), 1.11-1.06 (m, 2H).

### Preparation of (3-bromo-2-hydroxy-5-methyl-phenyl)-3-cyclopropyl-2-methyl-propane-1,3-dione

To a solution of (2-bromo-4-methyl-6-propanoyl-phenyl) cyclopropanecarboxylate (7.00 g, 22.5 mmol, 1 eq) in 2-MeTHF (150 mL) was added NaH (2.74 g, 68.6 mmol, 3.05 eq) in portions at 0° C. under N₂, then the reaction mixture was directly stirred at 80° C. (the oil bath was pre-heated to 80° C.) for 2 h. The reaction mixture was cooled to 25° C., poured into H₂O (100 mL) at 0° C. and then extracted with EtOAc (80 mL×3). The combined organic phases were washed with brine (100 mL×2), dried over Na₂SO₄, filtered and concentrated under reduced pressure to give the title compound (7.00 g, crude) as yellow oil, which was used directly for the next step without further purification.

### Preparation of 8-bromo-2-cyclopropyl-3,6-dimethyl-chromen-4-one

To a solution of 1-(3-bromo-2-hydroxy-5-methyl-phenyl)-3-cyclopropyl-2-methyl-propane-1,3-dione (7.00 g, 22.5 mmol, 1 eq) in AcOH (15 mL) was added H₂SO₄ (6.62 g, 67.5 mmol, 3.60 mL, 3 eq) dropwise at 25° C., then the reaction mixture was directly stirred at 800 (the oil bath was pre-heated to 80° C.) for 1 h. The reaction mixture was cooled to 25° C., poured into H₂O (30 mL) at 0° C. and then extracted with DCM (20 mL×3). The combined organic phases were washed with brine (40 mL×2), dried over Na₂SO₄, filtered and concentrated under reduced pressure to give a residue. The residue was purified by flash silica gel chromatography (Biotage®; 40 g SepaFlash®) Silica Flash Column, Eluent of 0~10% Ethyl acetate/Petroleum ether gradient @50 mL/min) to give the title compound (2.66 g, 9.07 mmol, 40.34% yield) as a yellow solid. ¹H NMR (CDCl₃, 400 MHz) δ 7.85 (d, J=2.0 Hz, 1H), 7.79-7.72 (m, 1H), 2.39 (s, 3H), 2.33-2.28 (m, 1H), 2.07 (s, 3H), 1.25-1.20 (m, 2H), 1.17-1.13 (m, 2H).

### Preparation of 2-cyclopropyl-8-[1-(4-hydroxybutoxy)vinyl]-3,6-dimethyl-chromen-4-one

To a mixture of 8-bromo-2-cyclopropyl-3,6-dimethyl-chromen-4-one (2.56 g, 8.73 mmol, 1 eq) and 4-vinyloxybutan-1-ol (20.30 g, 175 mmol, 21.6 mL, 20 eq) in H₂O (15 mL) were added DPPP (720 mg, 1.75 mmol, 0.2 eq), K₂CO₃ (1.81 g, 13.1 mmol, 1.5 eq) and Pd(OAc)₂ (196 mg, 873 μmol, 0.1 eq) in turns at 25° C., then the reaction mixture was directly stirred at 80° C. (the oil bath was pre-heated to 80° C.) for 10 h. The reaction mixture was cooled to 25° C., diluted with H₂O (15 mL) and then extracted with EtOAc (20 mL×3). The combined organic phases was washed with brine (30 mL×2), dried over Na₂SO₄, filtered and concentrated under reduced pressure to give the title compound (2.87 g, crude) as brown oil was used directly for the next step without purification.

### Preparation of 8-acetyl-2-cyclopropyl-3,6-dimethyl-chromen-4-one

To a solution of 2-cyclopropyl-8-[1-(4-hydroxybutoxy)vinyl]-3,6-dimethyl-chromen-4-one (2.87 g, 8.74 mmol, 1 eq) in H₂O (6 mL) was added H₃PO₄ (9.42 g, 96.1 mmol, 5.61 mL, 11 eq) dropwise at 25° C., the reaction mixture was stirred at 25° C. for 1 h. The reaction mixture was diluted with H₂O (10 mL) at 0° C. and then extracted with DCM (15 mL×3). The combined organic layers were washed with sat.aq NaHCO₃ to pH=8, then washed with brine (30 mL), dried over Na₂SO₄, filtered and concentrated under reduced pressure to give the title compound (2.15 g, 8.39 mmol, 95.99% yield) as a yellow solid. ¹H NMR (DMSO-d₆, 400 MHz) δ 7.99 (s, 1H), 7.93 (d, J=2.0 Hz, 1H), 2.63 (s, 3H), 2.43 (s, 3H), 2.33-2.26 (m, 1H) 2.09 (s, 3H), 1.28-1.21 (m, 2H), 1.12-1.09 (m, 2H).

### Preparation of 2-cyclopropyl-8-(1-hydroxyethyl)-3,6-dimethyl-chromen-4-one

To a solution of 8-acetyl-2-cyclopropyl-3,6-dimethyl-chromen-4-one (2.15 g, 8.39 mmol, 1 eq) in EtOH (15 mL)/DCM (15 mL) was added NaBH₄ (540 mg, 14.26 mmol, 1.7 eq) in portions at 0° C. under N₂, then the reaction mixture was warmed to 25° C. and stirred at 25° C. for 1 h under N₂. The reaction mixture was quenched with ice water (20 mL) and HCl (2 N, 2 mL) at 0° C. Nitrogen atmosphere protection was applied. The mixture was warmed up to 25° C. and then extracted with DCM (20 mL×3). The combined organic phases were washed with brine (25 mL×2), dried over Na₂SO₄, filtered and concentrated under reduced pressure to give a residue. The residue was purified by flash silica gel chromatography (Biotage®; 20 g SepaFlash® Silica Flash Column, Eluent of 0~15% Ethyl acetate/Petroleum ether gradient @60 L/mmin) to give the title compound (2.00 g, 7.74 mmol, 92.30% yield) as a white solid. ¹H NMR (CDCl₃, 400 MHz) δ 8.66 (s, 1H), 7.61 (s, 1H), 5.36 (d, J=4.0 Hz, 1H), 5.13-5.07 (m, 1H), 2.39 (s, 3H), 2.31-2.28 (m, 1H), 2.07 (s, 3H), 1.34 (d, J=6.4 Hz, 3H), 1.19-1.11 (m, 4H).

### Preparation of 8-(1-bromoethyl)-2-cyclopropyl-3,6-dimethyl-chromen-4-one

To a solution of 2-cyclopropyl-8-(1-hydroxyethyl)-3,6-dimethyl-chromen-4-one (2.00 g, 7.74 mmol, 1 eq) in DCM (25 mL)/DMF (367.84 mg, 5.03 mmol, 387.20 μL, 0.65 eq) was added dropwise sulfurous dibromide (2.09 g, 10.07 mmol, 779.84 μL, 1.3 eq) at 0° C., the reaction mixture was warmed to 25° C. and stirred at 25° C. for 1 h. The reaction mixture was poured into H₂O (10 mL) at 0° C. and extracted with DCM (15 mL×3). The combined organic layers were washed with sat.aq NaHCO₃ to pH=8, then washed with brine (10 mL), dried over Na₂SO₄, filtered and concentrated under reduced pressure to give a residue. The residue was purified by flash silica gel chromatography (Biotage®; 20 g SepaFlash® Silica Flash Column, Eluent of 0~8% Ethyl acetate/Petroleum ether gradient @50 mL/min) to give the title compound (2.00 g, 6.23 mmol, 80.42% yield) as a white solid. ¹H NMR (CDCl₃, 400 MHz) δ 7.95 (s, 1H), 7.53 (s, 1H), 5.57-5.52 (m, 1H), 2.44 (s, 3H), 2.20 (s, 3H), 2.18-2.16 (m, 1H), 2.12 (d, J=7.2 Hz, 3H), 1.48-1.45 (m, 1H), 1.36-1.33 (m, 1H), 1.18-1.15 (m, 2H).

### Preparation of 8-[1-[(2-bromo-6-chloro-3-pyridyl)amino]ethyl]-2-cyclopropyl-3,6-dimethyl-chromen-4-one

To a solution of 2-bromo-6-chloro-pyridin-3-amine (2.15 g, 10.39 mmol, 1.2 eq) in DMA (15 mL) was added NaH (519.29 mg, 12.98 mmol, 1.5 eq) at 0° C. in portions under N₂, the reaction mixture was warmed to 25° C. and stirred at 25° C. for 15 min. Then the reaction mixture was cooled to 0° C. and a solution of 8-(1-bromoethyl)-2-cyclopropyl-3,6-dimethyl-chromen-4-one (2.78 g, 8.65 mmol, 1 eq) in DMA (15 mL) was added dropwise at 0° C. under N₂. The resulting reaction mixture was warmed to 25° C. and stirred at 25° C. for 45 min. The reaction mixture was quenched by sat.aq.NH₄Cl (15 mL) at 0° C. and then extracted with EtOAc (20 mL×3). The combined organic phase was washed with brine (15 mL×2), dried over Na₂SO₄, filtered and concentrated under reduced pressure to give a residue. The crude product was triturated with EtOAc (10 mL) at 25° C. for 10 min to give the title compound (2.50 g, 5.58 mmol, 64.51% yield) as a light yellow solid. ¹H NMR (CDCl₃, 400 MHz) δ 7.90 (s, 1H), 7.30 (s, 1H), 6.96 (d, J=8.4 Hz, 1H), 6.39 (d, J=8.8 Hz, 1H), 4.86-4.81 (m, 1H), 4.78 (d, J=5.2 Hz, 1H), 2.37 (s, 3H), 2.22 (s, 3H), 2.21-2.20 (m, 1H), 1.64 (d, J=6.4 Hz, 3H), 1.21-1.16 (m, 4H).

### Preparation of 5-[6-chloro-3-[1-(2-cyclopropyl-3,6-dimethyl-4-oxo-chromen-8-yl) ethylamino]-2-pyridyl]-2-hydroxy-benzaldehyde

A mixture of 8-[1-[(2-bromo-6-chloro-3-pyridyl)amino]ethyl]-2-cyclopropyl-3,6-dimethyl-chromen-4-one (2.50 g, 5.58 mmol, 1 eq), 2-hydroxy-5-(4,4,5,5-tetramethyl-1,3,2-dioxa borolan-2-yl)benzaldehyde (1.66 g, 6.70 mmol, 1.2 eq), K₃PO₄ (2.37 g, 11.17 mmol, 2 eq), and Pd(dppf)Cl₂ (408.54 mg, 558.34 μmol, 0.1 eq) in dioxane (30 mL)/H₂O (6 mL) was degassed and purged with N₂ for 3 times, and then the mixture was stirred at 80° C. for 1 h under N₂ atmosphere. The reaction mixture was cooled to 25° C., diluted with H₂O (20 mL) and then extracted with EtOAc (25 mL×3). The combined organic layers were dried over Na₂SO₄, filtered and concentrated under reduced pressure to give a residue. The residue was purified by flash silica gel chromatography (Biotage®; 40 g SepaFlash® Silica Flash Column, Eluent of 0~35% Ethyl acetate/Petroleum ether gradient @60 mL/min) to give the title compound (2.00 g, 4.09 mmol, 73.26% yield) as a light yellow solid. ¹H NMR (CDCl₃, 400 MHz) δ 11.18 (s, 1H), 9.99 (s, 1H), 7.96 (d, J=2.0 Hz, 1H), 7.94-7.91 (m, 2H), 7.29 (d, J=2.0 Hz, 1H), 7.16 (d, J=8.8 Hz, 1H), 7.00 (d, J=8.4 Hz, 1H), 6.58 (d, J=8.8 Hz, 1H), 4.83-4.78 (m, 1H), 4.37 (s, 1H), 2.38 (s, 3H), 2.22 (s, 3H), 2.20-2.15 (m, 1H), 1.51 (d, J=6.4 Hz, 3H), 1.16-1.09 (m, 4H).

### Preparation of [4-[6-chloro-3-[1-(2-cyclopropyl-3,6-dimethyl-4-oxo-chromen-8-yl) ethylamino]-2-pyridyl]-2-formyl-phenyl]trifluoromethanesulfonate

To a solution of 5-[6-chloro-3-[1-(2-cyclopropyl-3,6-dimethyl-4-oxo-chromen-8-yl)ethyl amino]-2-pyridyl]-2-hydroxy-benzaldehyde (1.50 g, 3.07 mmol, 1 eq), TEA (620 mg, 6.14 mmol, 853 μL, 2 eq) and DMAP (37.4 mg, 306 μmol, 0.1 eq) in DCM (15 mL) was added 1,1,1-trifluoro-N-phenyl-N-(trifluoro methylsulfonyl)methanesulfonamide (1.42 g, 3.99 mmol, 1.3 eq) in portions at 25° C. The mixture was stirred at 25° C. for 1 h. The reaction mixture was partitioned between H₂O (50 mL) and DCM (100 mL). The organic phase was separated, washed with brine (50 mL), dried over Na₂SO₄, filtered and concentrated under reduced pressure to give a residue. The residue was purified by flash silica gel chromatography (BIOTAGE®; 40 g SepaFlash® Silica Flash Column, Eluent of 0~50% Ethyl acetate/Petroleum ether gradient @80 mL/min) to give the title compound (1.20 g, 1.93 mmol, 62.9% yield) as a yellow solid. ¹H NMR (DMSO-d₆, 400 MHz) δ 10.14 (s, 1H), 8.47 (d, J=2.0 Hz, 1H), 8.27 (dd, J=2.0, 8.4 Hz, 1H), 7.72 (d, J=8.4 Hz, 1H), 7.67 (d, J=1.2 Hz, 1H), 7.50 (d, J=2.0 Hz, 1H), 7.19 (d, J=8.4 Hz, 1H), 6.83 (d, J=8.8 Hz, 1H), 6.14 (d, J=6.8 Hz, 1H), 4.85-4.82 (m, 1H), 2.38-2.26 (m, 4H), 2.09 (s, 3H), 1.47 (d, J=6.8 Hz, 3H), 1.21-1.13 (m, 2H), 1.11-1.06 (m, 2H)

### Preparation of 5-[6-chloro-3-[1-(2-cyclopropyl-3,6-dimethyl-4-oxo-chromen-8-yl) ethylamino]-2-pyridyl]-2-(4,4,5,5-tetramethyl-1,3,2-dioxaborolan-2-yl)benzaldehyde

A mixture of [4-[6-chloro-3-[1-(2-cyclopropyl-3,6-dimethyl-4-oxo-chromen-8-yl) ethylamino]-2-pyridyl]-2-formyl-phenyl]trifluoromethanesulfonate (1.10 g, 1.77 mmol, 1 eq) and 4,4,5,5-tetramethyl-2-(4,4,5,5-tetramethyl-1,3,2-dioxaborolan-2-yl)-1,3,2-dioxaborolane (1.80 g, 7.09 mmol, 4 eq) in dioxane (11 mL) were added KOAc (434 mg, 4.43 mmol, 2.5 eq) and Pd(PPh₃)₂Cl₂ (124 mg, 177 μmol, 0.1 eq) in turns at 20° C. The reaction was degassed and purged with N₂ for 3 times, and then the reaction mixture was directly stirred at 80° C. (the oil-bath was pre-heated to 80° C.) for 0.5 h under N₂ atmosphere. The reaction mixture was cooled to 20° C., filtered and the filtrate was concentrated under reduced pressure to give a residue. The residue was purified by flash silica gel chromatography (BIOTAGE®; 40 g SepaFlash® Silica Flash Column, Eluent of 0~100% Ethyl acetate/Petroleum ether gradient @80 mL/min) to give a residue. The residue was triturated with MTBE (10 mL) at 25° C. for 30 min and then filtered. The filter cake was collected and dried in vacuo to give the title compound (600 mg, 1.0 mmol, 56.5% yield) as a yellow solid. ¹H NMR (DMSO-d₆, 400 MHz) δ 10.40 (s, 1H), 8.21 (d, J=1.2 Hz, 1H), 8.03 (dd, J=1.6, 7.6 Hz, 1H), 7.85 (d, J=7.6 Hz, 1H), 7.66 (d, J=1.6 Hz, 1H), 7.47 (d, J=2.0 Hz, 1H), 7.17 (d, J=8.4 Hz, 1H), 6.86 (d, J=8.8 Hz, 1H), 5.82 (d, J=6.8 Hz, 1H), 4.85-4.78 (m, 1H), 2.36-2.22 (m, 4H), 2.08 (s, 3H), 1.43 (d, J=6.4 Hz, 3H), 1.37 (s, 12H), 1.12-1.02 (m, 4H)

### Preparation of 8-[1-[[6-chloro-2-(1-hydroxy-2,3,1-benzoxazaborinin-6-yl)-3-pyridyl]amino]ethyl]-2-cyclopropyl-3,6-dimethyl-chromen-4-one

To a solution of 5-[6-chloro-3-[1-(2-cyclopropyl-3,6-dimethyl-4-oxo-chromen-8-yl)ethylamino]-2-pyridyl]-2-(4,4,5,5-tetramethyl-1,3,2-dioxaborolan-2-yl)benzaldehyde (500 mg, 834 mol, 1 eq) in EtOH (5 mL)/H₂O (5 mL) were added KOAc (163 mg, 1.67 mmol, 2 eq) and NH₂OH·HCl (116 mg, 1.67 mmol, 2 eq) in turns at 25° C., the reaction mixture was directly stirred at 50° C. (the oil-bath was pre-heated to 50° C.) for 2 h. The reaction mixture was cooled to 25° C. and the resulting suspension was filtered. The filter cake was triturated with MTBE (5 mL) at 25° C. for 30 min and filtered. The filter cake was collected and dried in vacuo to give the title compound (400 mg, 778 μmol, 93.2% yield) as an off-white solid. ¹H NMR (DMSO-d₆, 400 MHz) δ 9.48 (s, 1H), 8.72 (s, 1H), 8.16 (d, J=7.6 Hz, 1H), 8.07-8.02 (m, 2H), 7.66 (d, J=1.2 Hz, 1H), 7.48 (d, J=1.6 Hz, 1H), 7.18 (d, J=8.4 Hz, 1H), 6.87 (d, J=8.8 Hz, 1H), 5.85 (d, J=6.8 Hz, 1H), 4.87-4.71 (m, 1H), 2.32 (s, 3H), 2.29-2.21 (m, 1H), 2.08 (s, 3H), 1.44 (d, J=6.8 Hz, 3H), 1.10-0.98 (m, 4H).

### Preparation of 8-[(1S)-1-[[6-chloro-2-(1-hydroxy-2,3,1-benzoxazaborinin-6-yl)-3-pyri dyl]amino]ethyl]-2-cyclopropyl-3,6-dimethyl-chromen-4-one and 8-[(1R)-1-[[6-chloro-2-(1-hydroxy-2,3,1-benzoxazaborinin-6-yl)-3-pyridyl]amino]ethyl]-2-cyclopropyl-3,6-dimethyl-chromen-4-one

8-[1-[[6-chloro-2-(1-hydroxy-2,3,1-benzoxazaborinin-6-yl)-3-pyridyl]amino]ethyl]-2-cyclopropyl-3,6-dimethyl-chromen-4-one (400 mg) was separated by SFC (column: DAICEL CHIRALPAK AD (250 mm×30 mm×10 um); mobile phase: [CO2-IPA]; B %: 40%, isocratic elution mode) to give the title compound, Isomer 1 (126.2 mg, 245.6 μmol, 31.5% yield) as a white solid and the title compound, Isomer 2 (138.6 mg, 269 μmol, 34.6% yield) as an off-white solid. Isomer 1: ¹H NMR (DMSO-d₆, 400 MHz) δ 9.43 (s, 1H), 8.71 (s, 1H), 8.15 (d, J=7.6 Hz, 1H), 8.08-8.00 (m, 2H), 7.66 (s, 1H), 7.48 (d, J=1.6 Hz, 1H), 7.18 (d, J=8.4 Hz, 1H), 6.87 (d, J=8.8 Hz, 1H), 5.84 (d, J=7.2 Hz, 1H), 4.87-4.70 (m, 1H), 2.32 (s, 3H), 2.30-2.19 (m, 1H), 2.08 (s, 3H), 1.44 (d, J=6.8 Hz, 3H), 1.14-0.98 (m, 4H). MS (ESI): mass calcd. For C₂₈H₂₅BClN₃O₄ 513.16, m/z found 514.1 [M+H]⁺. HPLC: 98.23% (220 nm), 98.53% (254 nm). Chiral purity: 95.94% ee. Isomer 2: ¹H NMR (DMSO-d₆, 400 MHz) δ 9.44 (s, 1H), 8.72 (s, 1H), 8.15 (d, J=7.6 Hz, 1H), 8.07-8.00 (m, 2H), 7.66 (d, J=1.2 Hz, 1H), 7.48 (d, J=1.6 Hz, 1H), 7.18 (d, J=8.4 Hz, 1H), 6.87 (d, J=8.8 Hz, 1H), 5.84 (d, J=6.8 Hz, 1H), 4.86-4.73 (m, 1H), 2.32 (s, 3H), 2.29-2.22 (m, 1H), 2.08 (s, 3H), 1.44 (d, J=6.8 Hz, 3H), 1.12-0.99 (m, 4H). MS (ESI): mass calcd. For C₂₈H₂₅BClN₃O₄ 513.16, m/z found 514.1 [M+H]⁺. HPLC: 97.44% (220 nm), 97.75% (254 nm). Chiral purity: 98.08% ee

### Example 95

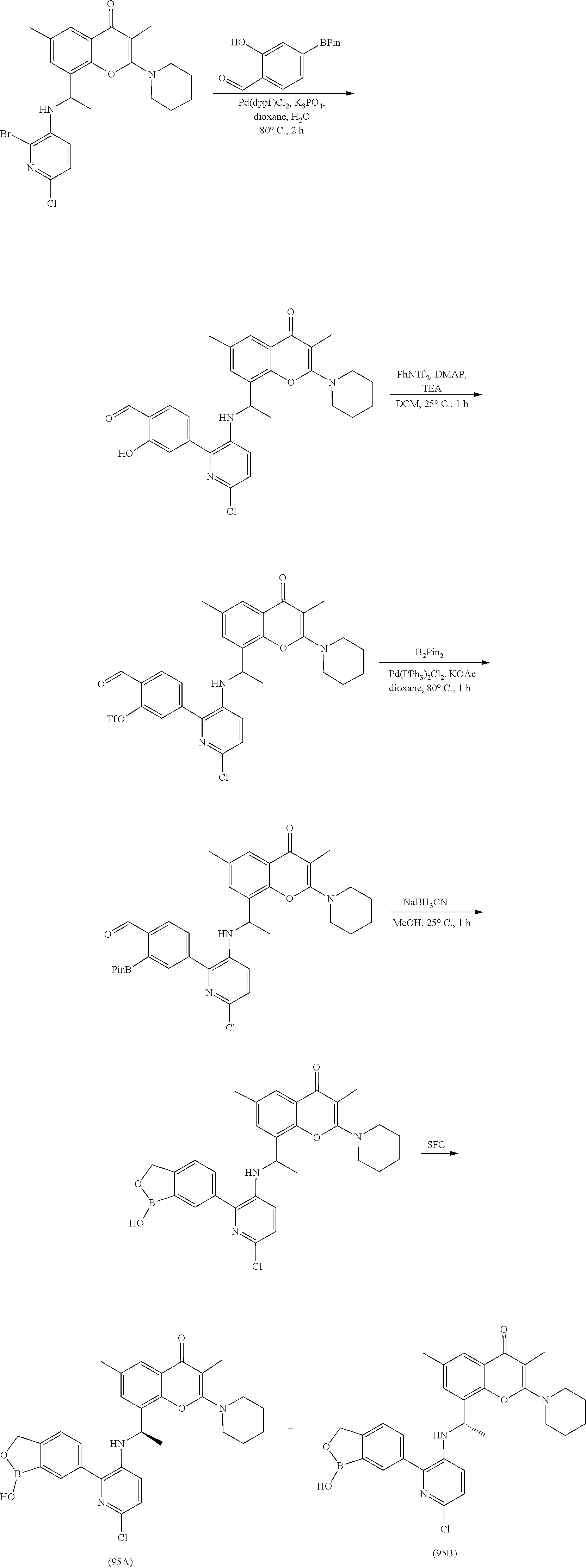

### Preparation of 4-[6-chloro-3-[1-[3,6-dimethyl-4-oxo-2-(1-piperidyl)chromen-8-yl]ethylamino]-2-pyridyl]-2-hydroxy-benzaldehyde

The title compound was prepared as a yellow solid (0.85 g, 1.60 mmol, 43.56% yield) according to the procedures described herein and the synthetic scheme, using 8-[1-[(2-bromo-6-chloro-3-pyridyl)amino]ethyl]-3,6-dimethyl-2-(1-piperidyl)chromen-4-one (1.80 g, 3.67 mmol, 1 eq) and 2-hydroxy-4-(4,4,5,5-tetramethyl-1,3,2-dioxaborolan-2-yl)benzaldehyde (0.91 g, 3.67 mmol, 1 eq). ¹H NMR (DMSO-d₆, 400 MHz) δ 10.88 (br s, 1H), 10.36 (s, 1H), 7.76 (d, J=8.0 Hz, 1H), 7.62 (s, 1H), 7.47 (s, 1H), 7.36 (s, 1H), 7.32 (d, J=6.8 Hz, 1H), 7.17 (d, J=8.8 Hz, 1H), 6.94 (d, J=8.4 Hz, 1H), 5.74 (d, J=6.8 Hz, 1H), 4.94 (q, J=6.8 Hz, 1H), 3.27-3.22 (m, 4H), 2.32 (s, 3H), 1.89 (s, 3H), 1.60-1.59 (m, 6H), 1.52 (d, J=6.4 Hz, 3H).

### Preparation of [5-[6-chloro-3-[1-[3,6-dimethyl-4-oxo-2-(1-piperidyl)chromen-8-yl]ethylamino]-2-pyridyl]-2-formyl-phenyl]trifluoromethanesulfonate

The title compound was prepared as a yellow solid (0.80 g, 1.20 mmol, 80.11% yield) according to the procedures described herein and the synthetic scheme, using 4-[6-chloro-3-[1-[3,6-dimethyl-4-oxo-2-(1-piperidyl)chromen-8-yl]ethylamino]-2-pyridyl]-2-hydroxy-benzaldehyde (0.80 g, 1.50 mmol, 1 eq) and 1,1,1-trifluoro-N-phenyl-N-(trifluoromethylsulfonyl)methanesulfonamide (1.07 g, 3.01 mmol, 2 eq). ¹H NMR (DMSO-d₆, 400 MHz) δ 10.16 (s, 1H), 8.24 (d, J=8.0 Hz, 1H), 8.14 (d, J=7.6 Hz, 1H), 7.94 (s, 1H), 7.63 (s, 1H), 7.45 (d, J=2.0 Hz, 1H), 7.21 (d, J=8.8 Hz, 1H), 6.20 (d, J=6.8 Hz, 1H), 4.97 (q, J=7.2 Hz, 1H), 3.32-3.30 (m, 4H), 2.31 (s, 3H), 1.91 (s, 3H), 1.62-1.59 (m, 6H), 1.51 (d, J=6.4 Hz, 3H).

### Preparation of 4-[6-chloro-3-[1-[3,6-dimethyl-4-oxo-2-(1-piperidyl)chromen-8-yl]ethylamino]-2-pyridyl]-2-(4,4,5,5-tetramethyl-1,3,2-dioxaborolan-2-yl)benzaldehyde

The title compound was prepared as a yellow solid (600 mg, 934.59 μmol, 82.75% yield) according to the procedures described herein and the synthetic scheme, using [5-[6-chloro-3-[1-[3,6-dimethyl-4-oxo-2-(1-piperidyl)chromen-8-yl]ethylamino]-2-pyridyl]-2-formyl-phenyl]trifluoromethanesulfonate (750 mg, 1.13 mmol, 1 eq) and B₂Pin₂ (860 mg, 3.39 mmol, 3 eq). The product was used directly for next step without further purification.

### Preparation of 8-[1-[[6-chloro-2-(1-hydroxy-3H-2,1-benzoxaborol-6-yl)-3-pyridyl]amino]ethyl]-3,6-dimethyl-2-(1-piperidyl)chromen-4-one

To a solution of 4-[6-chloro-3-[1-[3,6-dimethyl-4-oxo-2-(1-piperidyl)chromen-8-yl]ethylamino]-2-pyridyl]-2-(4,4,5,5-tetramethyl-1,3,2-dioxaborolan-2-yl)benzaldehyde (500 mg, 778.83 mol, 1 eq) in MeOH (5 mL) was added NaBH₃CN (74 mg, 1.17 mmol, 1.5 eq) at 0° C. in portions. The reaction mixture was warmed to 25° C. and stirred at 25° C. for 1 h. To the reaction mixture was added H₂O (5 mL) at 0° C., the resulting suspension was directly filtered. The filter cake was dried in vacuo and then triturated with CH₃CN/H₂O (6 mL, v/v=5/1) at 25° C. for 10 min to give the title compound (150 mg, 270.88 μmol, 34.78% yield) as a yellow solid. ¹H NMR (DMSO-d₆, 400 MHz) δ 9.31 (s, 1H), 8.07 (s, 1H), 7.82 (dd, J=1.2, 8.0 Hz, 1H), 7.61 (s, 1H), 7.53 (d, J=8.0 Hz, 1H), 7.46 (d, J=1.2 Hz, 1H), 7.14 (d, J=8.8 Hz, 1H), 6.93 (d, J=8.4 Hz, 1H), 5.46 (d, J=7.2 Hz, 1H), 5.06 (s, 2H), 4.97 (q, J=6.8 Hz, 1H), 3.34-3.32 (m, 4H), 2.32 (s, 3H), 1.89 (s, 3H), 1.58-1.57 (m, 6H), 1.50 (d, J=6.8 Hz, 3H).

### Preparation of 8-[(1S)-1-[[6-chloro-2-(1-hydroxy-3H-2,1-benzoxaborol-6-yl)-3-pyridyl]amino]ethyl]-3,6-dimethyl-2-(1-piperidyl)chromen-4-one and 8-[(1R)-1-[[6-chloro-2-(1-hydroxy-3H-2,1-benzoxaborol-6-yl)-3-pyridyl]amino]ethyl]-3,6-dimethyl-2-(1-piperidyl) chromen-4-one

8-[1-[[6-chloro-2-(1-hydroxy-3H-2,1-benzoxaborol-6-yl)-3-pyridyl]amino]ethyl]-3,6-dimethyl-2-(1-piperidyl)chromen-4-one (150 mg, 270.88 μmol, 1 eq) was separated by SFC (column: ChiralPak IH, 250×30 mm×10 um; mobile phase: [CO₂—IPA]; B %: 35%, isocratic elution mode) to give the title compound, Isomer 1 (57.7 mg, 105.38 μmol, 38.90% yield) as a white solid and the title compound, Isomer 2 (55.3 mg, 100.68 μmol, 37.17% yield) as a white solid. Isomer 1: ¹H NMR (DMSO-d₆, 400 MHz) δ 9.31 (s, 1H), 8.07 (s, 1H), 7.81 (dd, J=1.2, 8.0 Hz, 1H), 7.62 (s, 1H), 7.53 (d, J=8.0 Hz, 1H), 7.46 (d, J=2.0 Hz, 1H), 7.13 (d, J=8.4 Hz, 1H), 6.93 (d, J=8.4 Hz, 1H), 5.45 (d, J=7.2 Hz, 1H), 5.06 (s, 2H), 4.93 (quin, J=6.8 Hz, 1H), 3.33-3.17 (m, 4H), 2.32 (s, 3H), 1.89 (s, 3H), 1.58-1.57 (m, 6H), 1.51 (d, J=6.4 Hz, 3H). MS (ESI): mass calcd. For C₃₀H₃₁BClN₃O₄ 543.21, m/z found 544.3 [M+H]⁺. HPLC: 99.32% (220 nm), 99.04% (254 nm). Chiral purity: 100% ee. Isomer 2: ¹H NMR (DMSO-d₆, 400 MHz) δ 9.31 (s, 1H), 8.07 (s, 1H), 7.81 (dd, J=1.2, 8.0 Hz, 1H), 7.62 (s, 1H), 7.53 (d, J=8.0 Hz, 1H), 7.46 (d, J=2.0 Hz, 1H), 7.13 (d, J=8.4 Hz, 1H), 6.93 (d, J=8.8 Hz, 1H), 5.45 (d, J=7.2 Hz, 1H), 5.06 (s, 2H), 4.93 (q, J=6.8 Hz, 1H), 3.33-3.21 (m, 4H), 2.32 (s, 3H), 1.89 (s, 3H), 1.58-1.57 (m, 6H), 1.51 (d, J=6.8 Hz, 3H). MS (ESI): mass calcd. For C₃₀H₃₁BClN₃O₄ 543.21, m/z found 544.3 [M+H]⁺. HPLC: 99.02% (220 nm), 98.53% (254 nm). Chiral purity: 99.50% ee.

### Example 96

### Preparation of 8-bromo-2-(dimethylamino)-3,6-dimethyl-4H-chromen-4-one

A mixture of 8-bromo-3,6-dimethyl-chromen-4-one (10.00 g, 39.5 mmol, 1 eq), N-methylmethanamine; hydrochloride (16.10 g. 197 mmol, 5 eq), I₂ (25.0 g, 98.7 mmol, 19.9 mL, 2.5 eq), DMAP (482 mg, 3.95 mmol, 0.1 eq) and K₂CO₃ (43.60 g, 316 mmol, 8 eq) in DMA (200 mL) was degassed and purged with N₂ for 3 times at 20° C., and then the reaction mixture was heated to 80° C. and stirred at 80° C. for 5 h under N₂ atmosphere. The reaction mixture was cooled to 25° C., quenched by H₂O (600 mL) and then the resulting suspension was filtered to give the title compound (8.50 g, 28.70 mmol, 72.64% yield) as a yellow solid. ¹H NMR (CDCl₃, 400 MHz) δ 7.90 (d, J=1.2 Hz, 1H), 7.57 (d, J=2.0 Hz, 1H), 3.16 (s, 6H), 2.41 (s, 3H), 2.10 (s, 3H).

### Preparation of 2-(dimethylamino)-8-(1-(4-hydroxybutoxy)vinyl)-3,6-dimethyl-4H-chromen-4-one

A mixture of 8-bromo-2-(dimethylamino)-3,6-dimethyl-4H-chromen-4-one (8.00 g, 27.0 mmol, 1 eq), 4-(vinyloxy)butan-1-ol (62.70 g, 540 mmol, 66.8 mL, 20 eq), DPPP (2.23 g, 5.40 mmol, 0.2 eq), Pd(OAc)₂ (606 mg, 2.70 mmol, 0.1 eq) and K2CO₃ (5.60 g, 40.5 mmol, 1.5 eq) in H₂O (80 mL) was degassed and purged with N₂ for 3 times at 20° C., and then the mixture was heated to 100° C. and stirred at 100° C. for 2 h under N₂ atmosphere. The reaction mixture was cooled to 25° C. and directly extracted with EtOAc (100 mL×3). The combined organic layers were washed with brine (100 mL), dried over anhydrous Na₂SO₄, filtered and concentrated under reduced pressure to give the title compound (8.00 g, crude) as brown oil, which was used directly for next step without further purification.

### Preparation of 8-acetyl-2-(dimethylamino)-3,6-dimethyl-4H-chromen-4-one

A mixture of 2-(dimethylamino)-8-(1-(4-hydroxybutoxy)vinyl)-3,6-dimethyl-4H-chromen-4-one (17 g, 51.3 mmol, 1 eq) and concentrated H₃PO₄ (9.05 mL, 1.2 eq) in H₂O (200 mL) was degassed and purged with N₂ for 3 times at 20° C., and then the mixture was stirred at 20° C. for 3 h under N₂ atmosphere. The resulting suspension was filtered and the filter cake was washed with H₂O (50 mL×3), dried in vacuo to give the title compound (11.80 g, 45.51 mmol, 88.71% yield) as a yellow solid. ¹H NMR (CDCl₃, 400 MHz) δ 8.16 (d, J=2.0 Hz, 1H), 7.75 (d, J=2.0 Hz, 1H), 3.13 (s, 6H), 2.71 (s, 3H), 2.44 (s, 3H), 2.09 (s, 3H).

### Preparation of 2-(dimethylamino)-8-(1-hydroxyethyl)-3,6-dimethyl-4H-chromen-4-one

To a solution of 8-acetyl-2-(dimethylamino)-3,6-dimethyl-4H-chromen-4-one (11.80 g, 45.5 mmol, 1 eq) in EtOH (100 mL)/DCM (100 mL) was added NaBH₄ (3.58 g, 94.6 mmol, 2.08 eq) in portions at 0° C., the reaction mixture was stirred at 0° C. for 0.5 h. The reaction mixture was poured into sat.aq.NH₄Cl (200 mL) at 0° C. under N₂ atmosphere, and then extracted with DCM (200 mL×2). The combined organic layers were washed dried over anhydrous Na₂SO₄, filtered and concentrated under reduced pressure to give a residue. The crude product was triturated with MTBE (50 mL) at 20° C. for 30 min to give the title compound (11.00 g, 42.09 mmol, 92.50% yield) as a white solid. ¹H NMR (CDCl₃, 400 MHz) δ 7.63 (s, 1H), 7.39 (d, J=2.4 Hz, 1H), 5.29 (q, J=6.4 Hz, 1H), 3.09 (s, 6H), 2.31 (s, 3H), 2.06 (s, 3H), 1.51 (d, J=6.4 Hz, 3H).

### Preparation of 8-(1-bromoethyl)-2-(dimethylamino)-3,6-dimethyl-4H-chromen-4-one

To a solution of 2-(dimethylamino)-8-(1-hydroxyethyl)-3,6-dimethyl-chromen-4-one (10.00 g, 38.2 mmol, 1 eq) in DCM (100 mL)/DMF (279 mg, 3.83 mmol, 294 μL, 0.1 eq) was added SOBr₂ (11.9 g, 57.4 mmol, 4.45 mL, 1.5 eq) dropwise at 0° C., the reaction mixture was warmed to 20° C. and stirred at 20° C. for 0.5 h. The reaction mixture was quenched by H₂O (20 mL) at 0° C., and then extracted with DCM 600 mL (200 mL×3). The combined organic layers were washed with brine (300 mL), dried over anhydrous Na₂SO₄, filtered and concentrated under reduced pressure to give a residue. The residue was triturated with MTBE (100 mL) at 20° C. for 30 min to give the title compound (8.00 g, 24.68 mmol, 64.48% yield) as a yellow solid. ¹H NMR (CDCl₃, 400 MHz) δ 7.94 (s, 1H), 7.50 (d, J=2.0 Hz, 1H), 5.66 (q, J=7.2 Hz, 1H), 3.16 (s, 6H), 2.43 (s, 3H), 2.14 (d, J=6.8 Hz, 3H), 2.10 (s, 3H).

### Preparation of 8-(1-((2-bromo-6-chloropyridin-3-yl)amino)ethyl)-2-(dimethylamino)-3,6-dimethyl-4H-chromen-4-one

To a solution of 2-bromo-6-chloropyridin-3-amine (1.92 g, 9.25 mmol, 1.2 eq) in DMA (25 mL) was added NaH (462 mg, 11.5 mmol, 1.5 eq) in portions at 0° C., the resulting reaction mixture was stirred at 0° C. for 0.5 h. Then a solution of 8-(1-bromoethyl)-2-(dimethylamino)-3,6-dimethyl-4H-chromen-4-one (2.50 g, 7.71 mmol, 1 eq) in DMA (25 mL) was added dropwise at 0° C. and the resulting reaction mixture was stirred at 0° C. for additional 0.5 h. The reaction mixture was quenched by adding water (100 mL) at 0° C., and then resulting suspension was filtered to give the title compound (3.20 g, 7.10 mmol, 92.07% yield) as a brown solid. MS (ESI): mass calcd. For C₂₀H₂₁BrClN₃O₂ 449.05, m/z found 450.0 [M+H]⁺.

### Preparation of 5-(6-chloro-3-((1-(2-(dimethylamino)-3,6-dimethyl-4-oxo-4H-chromen-8-yl)ethyl)amino)pyridin-2-yl)-2-hydroxybenzaldehyde

A mixture of 8-(1-((2-bromo-6-chloropyridin-3-yl)amino)ethyl)-2-(dimethylamino)-3,6-dimethyl-4H-chromen-4-one (1.00 g, 2.22 mmol, 1 eq), 2-hydroxy-5-(4,4,5,5-tetramethyl-1,3,2-dioxaborolan-2-yl) benzaldehyde (825 mg, 3.33 mmol, 1.5 eq), Pd(PPh₃)₄ (256 mg, 221 μmol, 0.1 eq) and K₂CO₃ (613 mg, 4.44 mmol, 2 eq) in H₂O (1 mL)/dioxane (10 mL) was degassed and purged with N₂ for 3 times at 20° C., and then the mixture was heated to 95° C. and stirred at 95° C. for 1 h under N₂ atmosphere. The reaction mixture was filtered and the filtrate was concentrated under reduced pressure to give a residue. The residue was purified by flash silica gel chromatography (ISCO®; 12 g SepaFlash® Silica Flash Column, Eluent of 30~40% Ethyl acetate/Petroleum ether gradient @80 mL/min) to give the title compound (800 mg, 1.63 mmol, 73.30% yield) as a yellow solid. ¹H NMR (CDCl₃, 400 MHz) δ 11.17 (s, 1H), 9.98 (s, 1H), 7.96-7.96 (m, 1H), 7.88 (s, 1H), 7.68-7.66 (m, 1H), 7.27-7.25 (m, 1H), 7.16-7.14 (m, 1H), 7.01-6.98 (m, 1H), 6.65-6.63 (m, 1H), 4.92-4.85 (m, 1H), 4.43-4.42 (m, 1H), 3.06 (s, 6H), 2.37 (s, 3H), 2.10 (s, 3H), 1.56 (d, J=6.8 Hz, 3H).

### Preparation of 4-(6-chloro-3-((1-(2-(dimethylamino)-3,6-dimethyl-4-oxo-4H-chromen-8-yl)ethyl)amino)pyridin-2-yl)-2-formylphenyl trifluoromethanesulfonate

A mixture of 5-(6-chloro-3-((1-(2-(dimethylamino)-3,6-dimethyl-4-oxo-4H-chromen-8-yl)ethyl)amino)pyridin-2-yl)-2-hydroxybenzaldehyde (800 mg, 1.63 mmol, 1 eq), PhNTf₂ (1.16 g, 3.25 mmol, 2 eq), TEA (329 mg, 3.25 mmol, 452 μL, 2 eq) and DMAP (19.8 mg, 162 gmol, 0.1 eq) in DCM (10 mL) was degassed and purged with N₂ for 3 times at 20° C., and then the mixture was stirred at 20° C. for 1 h under N₂ atmosphere. The reaction mixture was filtered and the filtrate was concentrated under reduced pressure to give a residue. The residue was purified by flash silica gel chromatography (Biotage®; 20 g SepaFlash® Silica Flash Column, Eluent of 40-50% Ethyl acetate/Petroleum ether gradient @80 μL/min) to give the title compound (700 mg, 1.12 mmol, 68.98% yield) as a yellow solid. MS (ESI): mass calcd. For C₂₈H₂₅ClF₃N₃O₆S 623.11, m/z found 623.9 [M+H]⁺.

### Preparation of 5-(6-chloro-3-((1-(2-(dimethylamino)-3,6-dimethyl-4-oxo-4H-chromen-8-yl)ethyl)amino)pyridin-2-yl)-2-(4,4,5,5-tetramethyl-1,3,2-dioxaborolan-2-yl)benzaldehyde

A mixture of 4-(6-chloro-3-((1-(2-(dimethylamino)-3,6-dimethyl-4-oxo-4H-chromen-8-yl)ethyl)amino)pyridin-2-yl)-2-formylphenyl trifluoromethanesulfonate (600 mg, 961 μmol, 1 eq), B₂Pin₂ (1.22 g, 4.81 mmol, 5 eq), KOAc (188 mg, 1.92 mmol, 2 eq) and Pd(PPh₃)₂Cl₂ (67.4 mg, 96.1 mol, 0.1 eq) in dioxane (10 mL) was degassed and purged with N₂ for 3 times at 20° C., and then the mixture was heated to 80° C. and stirred at 80° C. for 1 h under N₂ atmosphere. The reaction mixture was cooled to 25° C., filtered and the filtrate was concentrated in vacuo. The residue was purified by flash silica gel chromatography (Biotage®; 4 g SepaFlash® Silica Flash Column, Eluent of 60~70% Ethyl acetate/Petroleum ether gradient @50 mL/min) to give the title compound (400 mg, 664.53 μmol, 69.11% yield) as yellow oil. MS (ESI): mass calcd. For C₃₃H₃₇BClN₃O₅ 601.25, m/z found 602.1 [M+H]⁺.

### Preparation of 8-(1-((6-chloro-2-(1-hydroxy-1H-benzo[d][1,2,6]oxazaborinin-6-yl)pyridin-3-yl)amino)ethyl)-2-(dimethylamino)-3,6-dimethyl-4H-chromen-4-one

A mixture of 5-(6-chloro-3-((1-(2-(dimethylamino)-3,6-dimethyl-4-oxo-4H-chromen-8-yl)ethyl)amino)pyridin-2-yl)-2-(4,4,5,5-tetramethyl-1,3,2-dioxaborolan-2-yl)benzaldehyde (200 mg, 332 mol, 1 eq), KOAc (65.2 mg, 664 μmol, 2 eq) and NH₂OH·HCl (46.1 mg, 664 μmol, 2 eq) in EtOH (2 mL) was degassed and purged with N₂ for 3 times at 20° C., and then the reaction mixture was stirred at 20° C. for 2 h under N₂ atmosphere. The reaction mixture was concentrated in vacuo and the residue was directly purified by prep-HPLC (column: 2_Phenomenex Gemini C18 75×40 mm×3 um; mobile phase: [H₂O (10 mM NH₄HCO₃)-AC□]; gradient:10%-50% B over 8.0 min) to give the title compound (65 mg, 124.82 μmol, 37.57% yield) as a white solid. MS (ESI): mass calcd. For C₂₇H₂₆BClN₄O₄ 516.17, m/z found 517.0 [M+H]⁺.

### Preparation of(S)-8-(1-((6-chloro-2-(1-hydroxy-1H-benzo[d][1,2,6]oxazaborinin-6-yl) pyridin-3-yl)amino)ethyl)-2-(dimethylamino)-3,6-dimethyl-4H-chromen-4-one & (R)-8-(1-((6-chloro-2-(1-hydroxy-JH-benzo[d][1,2,6]oxazaborinin-6-yl)pyridin-3-yl)amino)ethyl)-2-(dimethylamino)-3,6-dimethyl-4H-chromen-4-one

8-(1-((6-chloro-2-(1-hydroxy-1H-benzo[d][1,2,6]oxazaborinin-6-yl)pyridin-3-yl)amino)ethyl)-2-(dimethylamino)-3,6-dimethyl-4H-chromen-4-one (65 mg, 124 μmol, 1 eq) was purified by SFC (column: ChiralPak IH, 250×30 mm×10 um; mobile phase: [CO₂—IPA (0.1% NH₃H₂O)]; B %:55%, isocratic elution mode) to give the title compound, Isomer 1 (28.5 mg, 53.60 gmol, 42.62% yield) as white solid and the title compound, Isomer 2 (22.8 mg, 43.32 μmol, 34.45% yield) as white solid. Isomer 1: ¹H NMR (DMSO-d₆, 400 MHz) δ 9.51-9.44 (m, 1H), 8.69 (s, 1H), 8.19-8.14 (m, 1H), 8.04-8.01 (m, 2H), 7.60-7.59 (m, 1H), 7.40-7.40 (m, 1H), 7.19-7.09 (m, 1H), 6.95-6.87 (m, 1H), 5.85-5.84 (m, 1H), 4.98-4.89 (m, 1H), 3.10-2.93 (m, 6H), 2.31-2.30 (m, 3H), 1.95 (s, 3H), 1.49-1.47 (m, 3H). MS (ESI): mass calcd. For C₂₇H₂₆BClN₄O₄ 516.17, m/z found 517.1 [M+H]⁺. HPLC: 97.20% (220 nm), 97.69% (254 nm). Chiral purity: 100% ee. Isomer 2: ¹H NMR (DMSO-d₆, 400 MHz) δ 9.51-9.44 (m, 1H), 8.70 (s, 1H), 8.17-8.15 (m, 1H), 8.05-8.02 (m, 2H), 7.60 (s, 1H), 7.45-7.40 (m, 1H), 7.20-7.18 (m, 1H), 6.96-6.94 (m, 1H), 5.86-5.84 (m, 1H), 4.97-4.87 (m, 1H), 2.99-2.97 (m, 6H), 2.32-2.30 (m, 3H), 1.96 (s, 3H), 1.54-1.48 (m, 3H). MS (ESI): mass calcd. For C₂₇H₂₆BClN₄O₄ 516.17, m/z found 517.1 [M+H]⁺. HPLC: 98.20% (220 nm), 98.90% (254 nm). Chiral purity: 99.22% ee

### Example 97

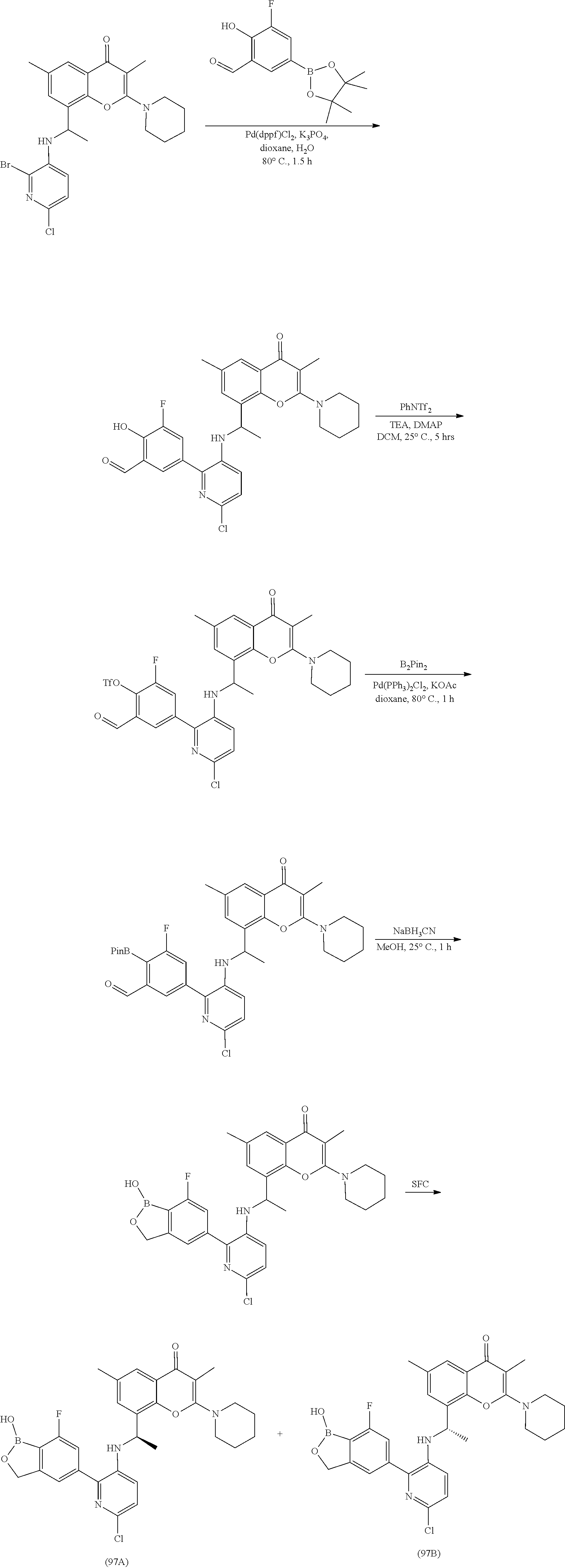

### Preparation of 5-[6-chloro-3-[1-[3,6-dimethyl-4-oxo-2-(1-piperidyl)chromen-8-yl]ethylamino]-2-pyridyl]-3-fluoro-2-hydroxy-benzaldehyde

A mixture of 8-[1-[(2-bromo-6-chloro-3-pyridyl)amino]ethyl]-3,6-dimethyl-2-(1-piperidyl) chromen-4-one (2.00 g, 4.07 mmol, 1 eq), 3-fluoro-2-hydroxy-5-(4,4,5,5-tetramethyl-1,3,2-dioxaborolan-2-yl)benzaldehyde (1.73 g, 6.52 mmol, 1.6 eq), K₃PO₄ (1.73 g, 8.15 mmol, 2 eq) and Pd(dppf)Cl₂ (298.16 mg, 407.48 μmol, 0.1 eq) in dioxane (30 mL)/H₂O (6 mL) was degassed and purged with N₂ for 3 times, and then the reaction mixture was heated to 80° C. and stirred at 80° C. for 1.5 h under N₂ atmosphere. The reaction mixture was cooled to 25° C., diluted with H₂O (25 mL) and extracted with EtOAc (20 mL×3). The combined organic layers were dried over Na₂SO₄, filtered and concentrated under reduced pressure to give a residue. The residue was purified by flash silica gel chromatography (Biotage®; 40 g SepaFlash® Silica Flash Column, Eluent of 0~30% Ethyl acetate/Petroleum ether gradient @60 mL/min) to give the title compound (1.30 g, 2.36 mmol, 58.00% yield) as a light yellow solid. ¹H NMR (CDCl₃, 400 MHz) δ 11.13 (s, 1H), 10.01 (s, 1H), 7.91 (s, 1H), 7.79 (s, 2H), 7.27 (s, 1H), 7.02 (d, J=8.8 Hz, 1H), 6.68 (d, J=8.4 Hz, 1H) 4.92-4.87 (m, 1H), 4.42 (s, 1H), 3.36-3.28 (m, 4H), 2.38 (s, 3H), 2.06 (s, 3H), 1.69 (s, 6H), 1.60 (d, J=6.8 Hz, 3H).

### Preparation of [4-[6-chloro-3-[1-[3,6-dimethyl-4-oxo-2-(1-piperidyl)chromen-8-yl]ethylamino]-2-pyridyl]-2-fluoro-6-formyl-phenyl]trifluoromethanesulfonate

To a solution of 5-[6-chloro-3-[1-[3,6-dimethyl-4-oxo-2-(1-piperidyl)chromen-8-yl]ethyl amino]-2-pyridyl]-3-fluoro-2-hydroxy-benzaldehyde (1.70 g, 3.09 mmol, 1 eq), TEA (626 mg, 6.18 mmol, 860.41 μL, 2 eq) and DMAP (37.76 mg, 309.08 μmol, 0.1 eq) in DCM (20 mL) was added 1,1,1-trifluoro-N-phenyl-N-(trifluoromethylsulfonyl)methanesulfonamide (2.21 g, 6.18 mmol, 2 eq) in one portion at 25° C., the reaction mixture was stirred at 25° C. for 5 h. The reaction mixture was cooled to 0° C., quenched by H₂O (20 mL) and extracted with DCM (15 mL×3). The combined organic layers were dried over Na₂SO₄, filtered and concentrated under reduced pressure to give a residue. The residue was purified by flash silica gel chromatography (Biotage®; 20 g SepaFlash® Silica Flash Column, Eluent of 0~35% Ethyl acetate/Petroleum ether gradient @60 mL/min) to give the title compound (1.50 g, 2.20 mmol, 71.15% yield) as a light yellow solid. ¹H NMR (CDCl₃, 400 MHz) δ 10.29 (s, 1H), 8.26 (s, 1H), 8.02-7.95 (m, 1H), 7.93 (s, 1H), 7.29 (s, 1H), 7.06 (d, J=8.4 Hz, 1H), 6.80 (d, J=8.4 Hz, 1H), 4.95-4.94 (m, 1H), 4.46-4.45 (m, 1H), 3.38-63.37 (m, 4H), 2.39 (s, 3H), 2.08 (s, 3H), 1.81-1.71 (m, 6H), 1.62-1.61 (s, 3H).

### Preparation of 5-[6-chloro-3-[1-[3,6-dimethyl-4-oxo-2-(1-piperidyl)chromen-8-yl]eth ylamino]-2-pyridyl]-3-fluoro-2-(4,4,5,5-tetramethyl-1,3,2-dioxaborolan-2-yl)benzaldehyde

To a mixture of [4-[6-chloro-3-[1-[3,6-dimethyl-4-oxo-2-(1-piperidyl) chromen-8-yl]ethylamino]-2-pyridyl]-2-fluoro-6-formyl-phenyl]trifluoromethanesulfonate (900 mg, 1.32 mmol, 1 eq), 4,4,5,5-tetramethyl-2-(4,4,5,5-tetramethyl-1,3,2-dioxaborolan-2-yl)-1,3,2-dioxa borolane (1.34 g, 5.28 mmol, 4 eq) and KOAc (259 mg, 2.64 mmol, 2 eq) in dioxane (10 mL) was added Pd(PPh₃)₂Cl₂ (92.6 mg, 131 μmol, 0.1 eq) in one portion at 20° C., the reaction was degassed and purged with N₂ for 3 times, and then stirred at 80° C. (the oil-bath was pre-heated to 80° C.) for 1 h under N₂ atmosphere. The reaction mixture was cooled to 20° C. and filtered. The filtrate was concentrated in vacuo and the residue was triturated with PE (20 mL) at 20° C. for 30 min. The solid was collected by filtration and dried in vacuo to give the title compound (1.5 g, crude) as a brown solid, which was used directly for next step without further purification.

### Preparation of 8-[1-[[6-chloro-2-(7-fluoro-1-hydroxy-3H-2,1-benzoxaborol-5-yl)-3-pyr idyl]amino]ethyl]-3,6-dimethyl-2-(1-piperidyl)chromen-4-one

To a solution of 5-[6-chloro-3-[1-[3,6-dimethyl-4-oxo-2-(1-piperidyl)chromen-8-yl]ethyl amino]-2-pyridyl]-3-fluoro-2-(4,4,5,5-tetramethyl-1,3,2-dioxaborolan-2-yl)benzaldehyde (900 mg, 1.36 mmol, 1 eq) in MeOH (9 mL) was added NaBH₃CN (257 mg, 4.09 mmol, 3 eq) in portions at 25° C., the mixture was stirred at 25° C. for 1 h. The reaction mixture was poured into H₂O (30 mL) and the resulting suspension was filtered. The filter cake was dissolved in EtOAc (40 mL), dried over Na₂SO₄, filtered and the filtrate was concentrated under reduced pressure. The residue was purified by flash silica gel chromatography (BIOTAGE®; 20 g SepaFlash® Silica Flash Column, Eluent of 0~30% MeOH/DCM @80 m/min) and then further purified by prep-HPLC (column: Waters Xbridge Prep OBD C18 150×40 mm×10 μm; mobile phase: [H₂O (10 mM NH₄HCO₃)-ACN]; gradient:30%-70% B over 8.0 min) to give the title compound (110 mg, 195 μmol, 14.3% yield) as a yellow solid. ¹H NMR (DMSO-d₆, 400 MHz) δ 9.37 (s, 1H), 7.62 (s, 1H), 7.53 (s, 1H), 7.45 (s, 1H), 7.35 (d, J=8.8 Hz, 1H), 7.17 (d, J=8.4 Hz, 1H), 6.97 (d, J=8.8 Hz, 1H), 5.72 (d, J=7.6 Hz, 1H), 5.10-4.97 (m, 2H), 4.93-4.89 (m, 1H), 3.25 (s, 4H), 2.32 (s, 3H), 1.89 (s, 3H), 1.59 (s, 6H), 1.51 (d, J=6.4 Hz, 3H)

### Preparation of 8-[(1S)-1-[[6-chloro-2-(7-fluoro-1-hydroxy-3H-2,1-benzoxaborol-5-yl)-3-pyridyl]amino]ethyl]-3,6-dimethyl-2-(1-piperidyl)chromen-4-one and 8-[(1R)-1-[[6-chloro-2-(7-fluoro-1-hydroxy-3H-2,1-benzoxaborol-5-yl)-3-pyridyl]amino]ethyl]-3,6-dimethyl-2-(1-piperidyl)chromen-4-one

8-[1-[[6-chloro-2-(7-fluoro-1-hydroxy-3H-2,1-benzoxaborol-5-yl)-3-pyridyl]amino]ethyl]-3,6-dimethyl-2-(1-piperidyl)chromen-4-one (150 mg) was purified by SFC (column: DAICEL CHIRALCEL OD (250 mm×30 mm, 10 um); mobile phase: [CO2-IPA]; B %:40%, isocratic elution mode) to give the title compound, Isomer 1 (47.6 mg, 84.7 μmol, 31.7% yield) as a white solid and the title compound, Isomer 2 (51.3 mg, 91.3 μmol, 34.2% yield) as a white solid. Isomer 1: ¹H NMR (DMSO-d₆, 400 MHz) δ 9.36 (s, 1H), 7.62 (s, 1H), 7.53 (s, 1H), 7.45 (d, J=2.0 Hz, 1H), 7.35 (d, J=8.8 Hz, 1H), 7.17 (d, J=8.4 Hz, 1H), 6.97 (d, J=8.4 Hz, 1H), 5.73 (d, J=7.6 Hz, 1H), 5.13-4.98 (m, 2H), 4.97-4.86 (m, 1H), 3.25 (d, J=3.6 Hz, 4H), 2.32 (s, 3H), 1.89 (s, 3H), 1.59 (s, 6H), 1.51 (d, J=6.4 Hz, 3H) MS (ESI): mass calcd. For C₃₀H₃₀BClFN₃O₄ 561.20, m/z found 562.2 [M+H]⁺. HPLC: 99.28% (220 nm), 99.22% (254 nm). Chiral purity: 100 ee %. Isomer 2: ¹H NMR (DMSO-d₆, 400 MHz) δ 9.35 (s, 1H), 7.62 (s, 1H), 7.53 (s, 1H), 7.45 (d, J=2.0 Hz, 1H), 7.35 (d, J=9.2 Hz, 1H), 7.17 (d, J=8.4 Hz, 1H), 6.97 (d, J=8.8 Hz, 1H), 5.73 (d, J=7.2 Hz, 1H), 5.13-4.98 (m, 2H), 4.96-4.87 (m, 1H), 3.25 (d, J=3.2 Hz, 4H), 2.32 (s, 3H), 1.89 (s, 3H), 1.59 (s, 6H), 1.51 (d, J=6.8 Hz, 3H) MS (ESI): mass calcd. For C₃₀H₃₀BClFN₃O₄ 561.20, m/z found 562.0 [M+H]⁺. HPLC: 99.55% (220 nm), 99.68% (254 nm). Chiral purity: 98.86 ee %

### Example 98

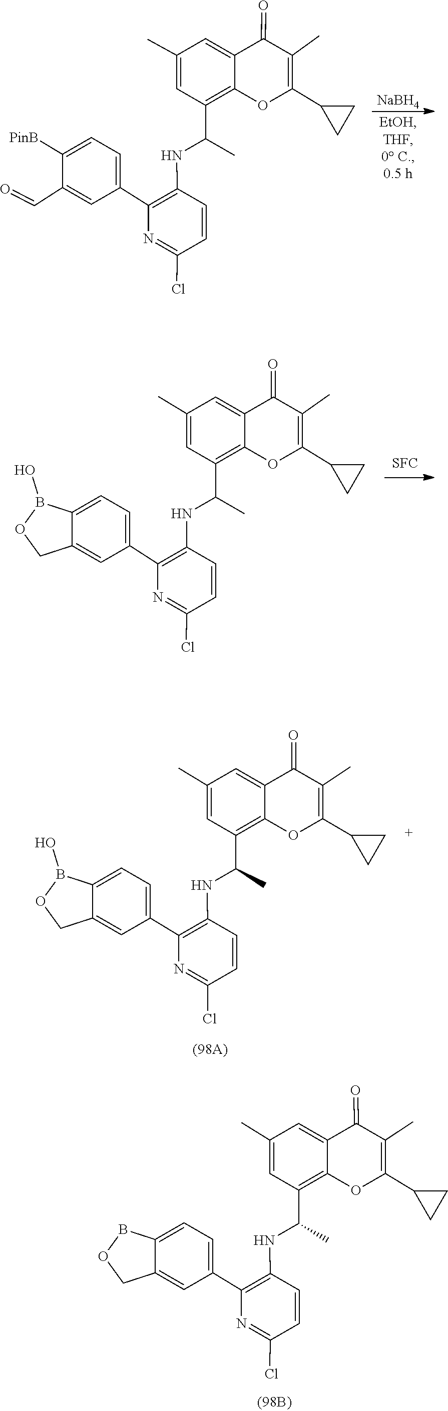

### Preparation of 8-[1-[[6-chloro-2-(1-hydroxy-3H-2,1-benzoxaborol-5-yl)-3-pyridyl]amino]ethyl]-2-cyclopropyl-3,6-dimethyl-chromen-4-one

To a solution of 5-[6-chloro-3-[1-(2-cyclopropyl-3,6-dimethyl-4-oxo-chromen-8-yl)ethylamino]-2-pyridyl]-2-(4,4,5,5-tetramethyl-1,3,2-dioxaborolan-2-yl)benzaldehyde (200 mg, 333 μmol, 1 eq) in THF (4 mL)/EtOH (2 mL) was added NaBH₄ (25 mg, 667 μmol, 2 eq) portion-wise at 0° C., the reaction mixture was stirred at 0° C. for 0.5 h. The reaction mixture was poured into ice water (20 mL) at 0° C., adjusted to pH=5 with 1N HCl and then extracted with EtOAc (20 mL×3). The combined organic phase was washed with brine (20 mL), dried over Na₂SO₄, filtered and concentrated under reduced pressure to give the title compound (167 mg, 333 μmol, 99.8% yield) as a yellow solid. ¹H NMR (DMSO-d₆, 400 MHz) δ 9.30 (s, 1H), 7.88-7.81 (m, 1H), 7.66 (s, 3H), 7.47 (d, J=1.6 Hz, 1H), 7.13 (d, J=8.4 Hz, 1H), 6.83 (d, J=8.8 Hz, 1H), 5.52 (d, J=6.8 Hz, 1H), 5.04 (d, J=3.2 Hz, 2H), 4.88-4.72 (m, 1H), 2.34-2.23 (m, 4H), 2.08 (s, 3H), 1.44 (d, J=6.8 Hz, 3H), 1.14-1.00 (m, 4H)

### Preparation of 8-[(1S)-1-[[6-chloro-2-(1-hydroxy-3H-2,1-benzoxaborol-5-yl)-3-pyridyl]amino]ethyl]-2-cyclopropyl-3,6-dimethyl-chromen-4-one and 8-[(1R)-1-[[6-chloro-2-(1-hydroxy-3H-2,1-benzoxaborol-5-yl)-3-pyridyl]amino]ethyl]-2-cyclopropyl-3,6-dimethyl-chromen-4-one

8-[1-[[6-chloro-2-(1-hydroxy-3H-2,1-benzoxaborol-5-yl)-3-pyridyl]amino]ethyl]-2-cyclopropyl-3,6-dimethyl-chromen-4-one (150 mg) was purified by SFC (column: ChiralPak IH, 250×30 mm×10 um; mobile phase: [CO2-EtOH]; B %:38%, isocratic elution mode) to give the title compound, Isomer 1 (60.9 mg, 121 μmol, 40.6% yield) as a white solid and the title compound, Isomer 2 (54.3 mg, 108 μmol, 36.2% yield) as an off-white solid. Isomer 1: ¹H NMR (DMSO-d₆, 400 MHz) δ 9.29 (s, 1H), 7.84 (d, J=8.0 Hz, 1H), 7.66 (d, J=2.8 Hz, 3H), 7.47 (d, J=2.0 Hz, 1H), 7.14 (d, J=8.4 Hz, 1H), 6.83 (d, J=8.8 Hz, 1H), 5.51 (d, J=6.8 Hz, 1H), 5.04 (d, J=3.6 Hz, 2H), 4.84-4.78 (m, 1H), 2.32 (s, 3H), 2.30-2.23 (m, 1H), 2.08 (s, 3H), 1.44 (d, J=6.8 Hz, 3H), 1.17-0.97 (m, 4H) MS (ESI): mass calcd. For C₂₈H₂₆BClN₂O₄ 500.17, m/z found 501.1 [M+H]⁺. HPLC: 97.36% (220 nm), 97.39% (254 nm). Chiral purity: 99.84 ee %. Isomer 2: ¹H NMR (DMSO-d₆, 400 MHz) δ 9.29 (s, 1H), 7.84 (d, J=8.0 Hz, 1H), 7.66 (d, J=2.4 Hz, 3H), 7.47 (d, J=2.0 Hz, 1H), 7.14 (d, J=8.8 Hz, 1H), 6.83 (d, J=8.4 Hz, 1H), 5.52 (d, J=6.8 Hz, 1H), 5.04 (d, J=3.6 Hz, 2H), 4.83-4.79 (m, 1H), 2.32 (s, 3H), 2.30-2.22 (m, 1H), 2.08 (s, 3H), 1.44 (d, J=6.8 Hz, 3H), 1.14-1.01 (m, 4H) MS (ESI): mass calcd. For C₂₈H₂₆BClN₂O₄ 500.17, m/z found 501.1 [M+H]⁺. HPLC: 97.31% (220 nm), 97.45% (254 nm). Chiral purity: 99.62 ee %

### Example 99

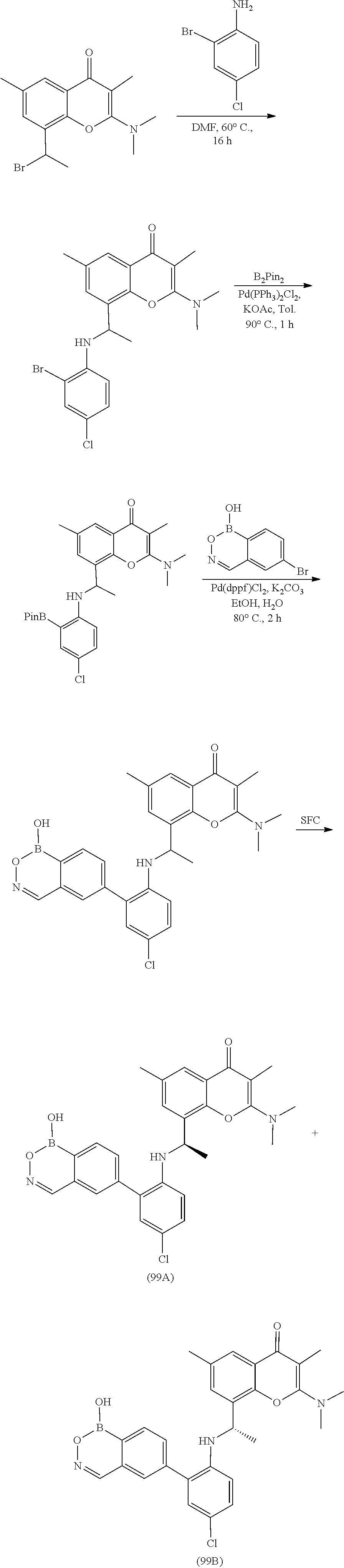

### Preparation of 8-[1-(2-bromo-4-chloro-anilino)ethyl]-2-(dimethylamino-3,6-dimethyl-chromen-4-one

The title compound was prepared as an off-white solid (1.90 g, 4.22 mmol, 68.48% yield) according to the procedures described herein and the synthetic scheme, using 8-(1-bromoethyl)-2-(dimethylamino)-3,6-dimethyl-chromen-4-one (2.00 g, 6.17 mmol, 1 eq) and 2-bromo-4-chloro-aniline (2.80 g, 13.57 mmol, 2.2 eq). ¹H NMR (DMSO-d₆, 400 MHz) δ 7.62 (d, J=1.2 Hz, 1H), 7.53 (d, J=2.4 Hz, 1H), 7.37 (d, J=2.0 Hz, 1H), 7.10 (dd, J=2.4, 8.8 Hz, 1H), 6.39 (d, J=9.2 Hz, 1H), 5.47 (d, J=7.2 Hz, 1H), 5.06-4.99 (m, 1H), 3.10 (s, 6H), 2.30 (s, 3H), 1.98 (s, 3H), 1.61 (d, J=6.8 Hz, 3H).

### Preparation of 8-[1-[4-chloro-2-(4,4,5,5-tetramethyl-1,3,2-dioxaborolan-2-yl)anilino]ethyl]-2-(dimethylamino)-3,6-dimethyl-chromen-4-one

The title compound was prepared as a brown solid (1.23 g, 2.48 mmol, 85.65% yield) according to the procedures described herein and the synthetic scheme, using 8-[1-(2-bromo-4-chloro-anilino)ethyl]-2-(dimethylamino)-3,6-dimethyl-chromen-4-one (1.30 g, 2.89 mmol, 1 eq) and 4,4,5,5-tetramethyl-2-(4,4,5,5-tetramethyl-1,3,2-dioxaborolan-2-yl)-1,3,2-dioxaborolane (3.67 g, 14.45 mmol, 5 eq). ¹H NMR (DMSO-d₆, 400 MHz) δ 7.63 (d, J=1.2 Hz, 1H), 7.34 (d, J=2.8 Hz, 1H), 7.32 (d, J=2.0 Hz, 1H), 7.17 (dd, J=2.4, 8.8 Hz, 1H), 6.34 (d, J=8.8 Hz, 2H), 5.00-4.97 (m, 1H), 3.10 (s, 6H), 2.29 (s, 3H), 1.98 (s, 3H), 1.55 (d, J=6.4 Hz, 3H), 1.33 (d, J=2.4 Hz, 12H).

### Preparation of 8-[1-[4-chloro-2-(1-hydroxy-2,3,1-benzoxazaborinin-6-yl)anilino]ethyl]-2-(dimethylamino)-3,6-dimethyl-chromen-4-one

The title compound was prepared as a white solid (350 mg, 678.56 μmol, 33.71% yield) according to the procedures described herein and the synthetic scheme, using 8-[1-[4-chloro-2-(4,4,5,5-tetramethyl-1,3,2-dioxaborolan-2-yl)anilino]ethyl]-2-(dimethylamino)-3,6-dimethyl-chromen-4-one (1.00 g, 2.01 mmol, 1 eq) and 6-bromo-1-hydroxy-2,3,1-benzoxazaborinine (545 mg, 2.42 mmol, 1.2 eq).

### Preparation of 8-[(1S)-1-[4-chloro-2-(1-hydroxy-2,3,1-benzoxazaborinin-6-yl)anilino]ethyl]-2-(dimethylamino)-3,6-dimethyl-chromen-4-one and 8-[(1R)-1-[4-chloro-2-(1-hydroxy-2,3,1-benzoxazaborinin-6-yl)anilino]ethyl]-2-(dimethylamino)-3,6-dimethyl-chromen-4-one

8-[1-[4-chloro-2-(1-hydroxy-2,3,1-benzoxazaborinin-6-yl)anilino]ethyl]-2-(dimethylamino)-3,6-dimethyl-chromen-4-one (500 mg, 969.38 μmol, 1 eq) was separated by SFC (column: ChiralPak IH, 250×30 mm×10 um; mobile phase: [CO2-IPA]; B %:53%, isocratic elution mode) to give the title compound, Isomer 1 (236 mg, 457.55 μmol, 47.20% yield) as an off-white solid and the title compound, Isomer 2 (245.3 mg, 475.58 μmol, 49.06% yield) as an off-white solid. Isomer 1: ¹H NMR (DMSO-d₆, 400 MHz) δ 9.45 (s, 1H), 8.65 (s, 1H), 8.14 (d, J=7.6 Hz, 1H), 7.85 (dd, J=1.6, 8.0 Hz, 1H), 7.83 (s, 1H), 7.59 (d, J=1.2 Hz, 1H), 7.39 (d, J=2.0 Hz, 1H), 7.14 (dd, J=2.4, 8.8 Hz, 1H), 7.08 (d, J=2.8 Hz, 1H), 6.52 (d, J=8.8 Hz, 1H), 5.27 (d, J=7.2 Hz, 1H), 4.96-4.82 (m, 1H), 2.98 (s, 6H), 2.31 (s, 3H), 1.96 (s, 3H), 1.43 (d, J=6.4 Hz, 3H). MS (ESI): mass calcd. For C₂₈H₂₇BClN₃O₄ 515.18, m/z found 516.1 [M+H]⁺. HPLC: 98.83% (220 nm), 98.80% (254 nm). Chiral purity: 100% ee. Isomer 2: ¹H NMR (DMSO-d₆, 400 MHz) δ 9.46 (s, 1H), 8.65 (s, 1H), 8.14 (d, J=7.6 Hz, 1H), 7.88-7.84 (m, 1H), 7.83 (s, 1H), 7.59 (d, J=1.2 Hz, 1H), 7.39 (d, J=2.0 Hz, 1H), 7.14 (dd, J=2.4, 8.8 Hz, 1H), 7.08 (d, J=2.4 Hz, 1H), 6.52 (d, J=8.8 Hz, 1H), 5.27 (d, J=7.2 Hz, 1H), 4.98-4.84 (m, 1H), 2.98 (s, 6H), 2.31 (s, 3H), 1.96 (s, 3H), 1.43 (d, J=6.4 Hz, 3H). MS (ESI): mass calcd. For C₂₈H₂₇BClN₃O₄ 515.18, m/z found 516.1 [M+H]⁺. HPLC: 99.62% (220 nm), 99.47% (254 nm). Chiral purity: 98.74% ee

### Example 100

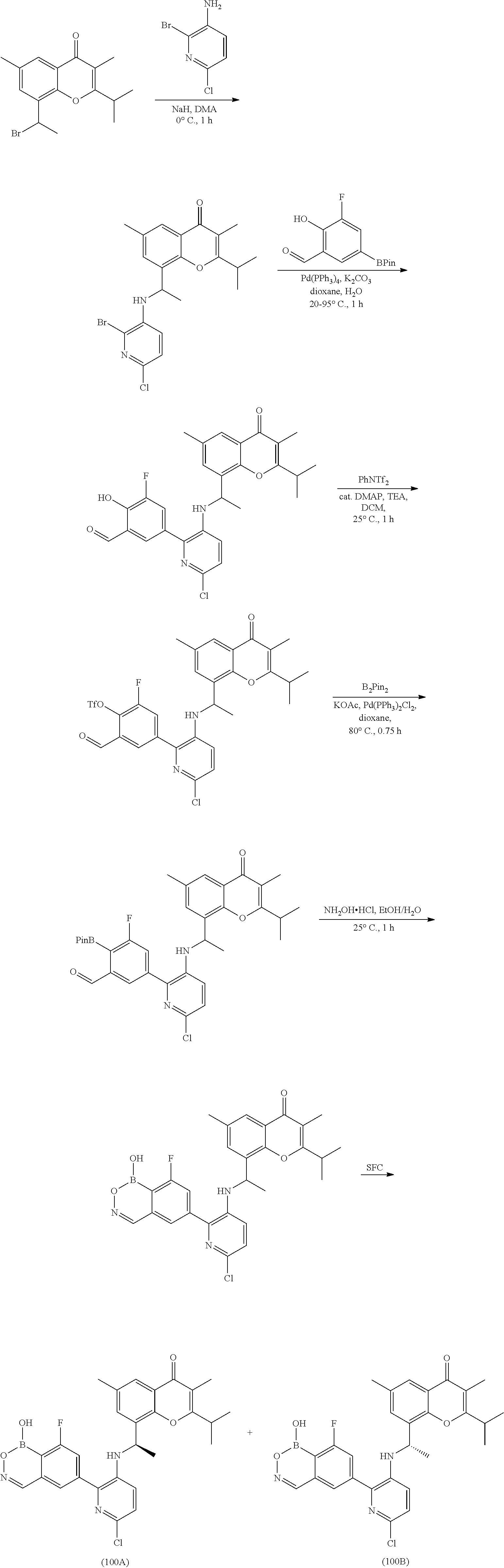

### Preparation of 8-[1-[(2-bromo-6-chloro-3-pyridyl)amino]ethyl]-2-isopropyl-3,6-dimethyl-chromen-4-one

The title compound was prepared as a brown solid (1.40 g, 3.11 mmol, 50.31% yield) according to the procedures described herein and the synthetic scheme, using 2-bromo-6-chloro-pyridin-3-amine (1.28 g, 6.19 mmol, 1 eq) and 8-(1-bromoethyl)-2-isopropyl-3,6-dimethyl-chromen-4-one (2.00 g, 6.19 mmol, 1 eq). ¹H NMR (DMSO-d₆, 400 MHz) δ 7.91 (s, 1H), 7.34 (d, J=2.0 Hz, 1H), 6.96 (d, J=8.4 Hz, 1H), 6.45 (d, J=8.4 Hz, 1H), 5.04-4.99 (m, 1H), 4.83-4.82 (m, 1H), 3.36-3.28 (m, 1H), 2.39 (s, 3H), 2.12 (s, 3H), 1.70 (d, J=6.4 Hz, 3H), 1.37 (d, J=6.8 Hz, 6H).

### Preparation of 5-[6-chloro-3-[1-(2-isopropyl-3,6-dimethyl-4-oxo-chromen-8-yl) ethylamino]-2-pyridyl]-3-fluoro-2-hydroxy-benzaldehyde

The title compound was prepared as a light yellow solid (1.05 g, 2.06 mmol, 74.23% yield) according to the procedures described herein and the synthetic scheme, using 8-[1-[(2-bromo-6-chloro-3-pyridyl)amino]ethyl]-2-isopropyl-3,6-dimethyl-chromen-4-one (1.25 g, 2.78 mmol, 1 eq) and 3-fluoro-2-hydroxy-5-(4,4,5,5-tetramethyl-1,3,2-dioxaborolan-2-yl)benzaldehyde (1.11 g, 4.17 mmol, 1.5 eq). ¹H NMR (DMSO-d₆, 400 MHz) δ 10.34 (s, 1H), 7.89-7.84 (m, 2H), 7.68 (s, 1H), 7.56 (s, 1H), 7.13 (d, J=8.8 Hz, 1H), 6.84 (d, J=8.4 Hz, 1H), 5.88 (d, J=7.2 Hz, 1H), 5.03-4.97 (m, 1H), 3.30-3.27 (m, 1H), 2.34 (s, 3H), 1.99 (s, 3H), 1.27-1.25 (m, 3H), 1.22-1.20 (m, 3H), 1.16 (s, 3H).

### Preparation of [4-[6-chloro-3-[1-(2-isopropyl-3,6-dimethyl-4-oxo-chromen-8-yl)ethyl amino]-2-pyridyl]-2-fluoro-6-formyl-phenyl]trifluoromethanesulfonate

The title compound was prepared as a yellow solid (0.84 g, 1.31 mmol, 63.52% yield) according to the procedures described herein and the synthetic scheme, using 5-[6-chloro-3-[1-(2-isopropyl-3,6-dimethyl-4-oxo-chromen-8-yl)ethylamino]-2-pyridyl]-3-fluoro-2-hydroxy-benzaldehyde (1.05 g, 2.06 mmol, 1 eq) and PhNTf₂ (1.11 g, 3.09 mmol, 1.5 eq). ¹H NMR (DMSO-d₆, 400 MHz) δ 10.14 (s, 1H), 8.34-8.30 (m, 3H), 7.69 (s, 1H), 7.56 (d, J=2.0 Hz, 1H), 6.87 (d, J=8.8 Hz, 1H), 6.31 (d, J=0.6.8H, 1H), 5.05-4.98 (m, 1H), 3.30-3.29 (m, 1H), 2.34 (s, 3H), 2.00 (s, 3H), 1.54 (d, J=6.4 Hz, 3H), 1.30-1.28 (m, 3H), 1.26-1.24 (m, 3H).

### Preparation of 5-[6-chloro-3-[1-(2-isopropyl-3,6-dimethyl-4-oxo-chromen-8-yl) ethylamino]-2-pyridyl]-3-fluoro-2-(4,4,5,5-tetramethyl-1,3,2-dioxaborolan-2-yl)benzaldehyde

The title compound was prepared as a brown solid (1.94 g, crude) according to the procedures described herein and the synthetic scheme, using [4-[6-chloro-3-[1-(2-isopropyl-3,6-dimethyl-4-oxo-chromen-8-yl)ethylamino]-2-pyridyl]-2-fluoro-6-formyl-phenyl]trifluoromethanesulfonate (0.84 g, 1.31 mmol, 1 eq) and B₂Pin₂ (998 mg, 3.93 mmol, 3 eq). The product was used directly for next step without further purification.

### Preparation of 8-(1-((6-chloro-2-(8-fluoro-1-hydroxy-JH-benzo[d][1,2,6]oxazaborinin-6-yl)pyridin-3-yl)amino)ethyl)-2-isopropyl-3,6-dimethyl-4H-chromen-4-one

The title compound was prepared as a pale-yellow solid (150 mg, 281.01 gmol, 8.97% yield) according to the procedures described herein and the synthetic scheme, using 5-[6-chloro-3-[1-(2-isopropyl-3,6-dimethyl-4-oxo-chromen-8-yl)ethylamino]-2-pyridyl]-3-fluoro-2-(4,4,5,5-tetramethyl-1,3,2-dioxaborolan-2-yl)benzaldehyde (1.94 g, 3.13 mmol, 1 eq) and hydroxylamine;hydrochloride (327 mg, 4.70 mmol, 1.5 eq). ¹H NMR (DMSO-d₆, 400 MHz) δ 9.35 (s, 1H), 7.62 (d, J=1.2 Hz, 1H), 7.52 (s, 1H), 7.44 (d, J=2.0 Hz, 1H), 7.35 (d, J=8.8 Hz, 1H), 7.17 (d, J=8.8 Hz, 1H), 6.95 (d, J=8.4 Hz, 1H), 5.74 (d, J=7.2 Hz, 1H), 5.07-5.02 (m, 2H), 4.98-4.91 (m, 1H), 3.30-3.28 (m, 4H), 2.32 (s, 3H), 1.90 (s, 3H), 1.51 (d, J=6.8 Hz, 3H), 1.41 (t, J=5.2 Hz, 4H), 0.97 (s, 6H).

### Preparation of 8-[(1S)-1-[[6-chloro-2-(8-fluoro-1-hydroxy-2,3,1-benzoxazaborinin-6-yl)-3-pyridyl]amino]ethyl]-2-isopropyl-3,6-dimethyl-chromen-4-one and 8-[(1R)-1-[[6-chloro-2-(8-fluoro-1-hydroxy-2,3,1-benzoxazaborinin-6-yl)-3-pyridyl]amino]ethyl]-2-isopropyl-3,6-dimethyl-chromen-4-one

8-[1-[[6-chloro-2-(8-fluoro-1-hydroxy-2,3,1-benzoxazaborinin-6-yl)-3-pyridyl]amino]ethyl]-2-isopropyl-3,6-dimethyl-chromen-4-one (0.13 g, 243.54 μmol, 1 eq) was separated by SFC (column: ChiralPak IH, 250×30 mm×10 um; mobile phase: [CO₂-MeOH (0.1% IPA]; B %:20%, isocratic elution mode) to give the title compound, Isomer 1 (17.2 mg, 32.12 μmol, 42.86% yield) as a white solid and the title compound, Isomer 2 (18.8 mg, 35.11 μmol, 46.85% yield) as a white solid. Isomer 1: ¹H NMR (DMSO-d₆, 400 MHz) δ 8.72 (s, 1H), 7.90 (s, 1H), 7.77 (s, 1H), 7.68 (s, 1H), 7.56 (s, 1H), 7.20 (d, J=8.8 Hz, 1H), 6.89 (d, J=8.8 Hz, 1H), 6.02 (d, J=7.2 Hz, 1H), 5.00 (t, J=6.8 Hz, 1H), 3.28-3.27 (m, 1H), 2.34 (s, 3H), 1.99 (s, 3H), 1.52 (d, J=6.4 Hz, 3H), 1.26 (d, J=6.8 Hz, 3H), 1.23 (d, J=6.8 Hz, 3H). MS (ESI): mass calcd. For C₂₈H₂₆BClFN₃O₄ 533.17, m/z found 534.1 [M+H]⁺. HPLC: 99.67% (220 nm), 99.64% (254 nm). Chiral purity: 100% ee. Isomer 2: ¹H NMR (DMSO-d₆, 400 MHz) δ 8.72 (s, 1H), 7.90 (s, 1H), 7.77 (s, 1H), 7.68 (s, 1H), 7.56 (s, 1H), 7.20 (d, J=8.8 Hz, 1H), 6.89 (d, J=8.8 Hz, 1H), 6.02 (d, J=7.2 Hz, 1H), 5.00 (t, J=6.8 Hz, 1H), 3.28-3.27 (m, 1H), 2.34 (s, 3H), 1.99 (s, 3H), 1.52 (d, J=6.4 Hz, 3H), 1.26 (d, J=6.8 Hz, 3H), 1.23 (d, J=6.8 Hz, 3H). MS (ESI): mass calcd. For C₂₈H₂₆BClFN₃O4 533.17, m/z found 534.1 [M+H]⁺. HPLC: 99.69% (220 nm), 99.65% (254 nm). Chiral purity: 97.12% ee

### Example 101

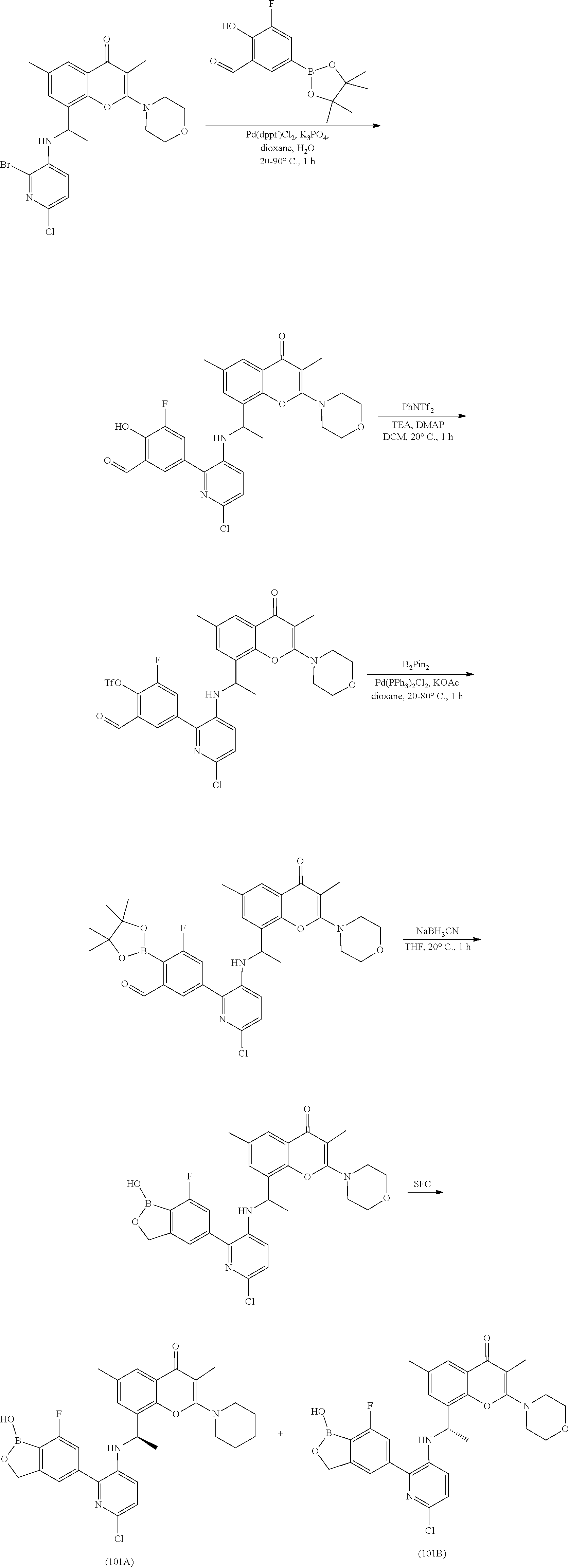

### Preparation of 5-(6-chloro-3-((1-(3,6-dimethyl-2-morpholino-4-oxo-4H-chromen-8-yl) ethyl)amino)pyridin-2-yl)-3-fluoro-2-hydroxybenzaldehyde

The title compound was prepared as a yellow solid (2.00 g, 3.62 mmol, 51.01% yield) according to the procedures described herein and the synthetic scheme, using 8-(1-((2-bromo-6-chloropyridin-3-yl)amino)ethyl)-3,6-dimethyl-2-morpholino-4H-chromen-4-one (3.50 g, 7.10 mmol, 1 eq) and 3-fluoro-2-hydroxy-5-(4,4,5,5-tetramethyl-1,3,2-dioxaborolan-2-yl)benzaldehyde (2.27 g, 8.52 mmol, 1.2 eq). ¹H NMR (CDCl₃, 400 MHz) δ11.12 (s, 1H), 10.01 (d, J=1.6 Hz, 1H), 7.91 (d, J=1.6 Hz, 1H), 7.81-7.79 (m, 2H), 7.30 (d, J=2.0 Hz, 1H), 7.02 (d, J=8.4 Hz, 1H), 6.65 (d, J=8.8 Hz, 1H), 4.91-4.86 (m, 1H), 4.41 (d, J=5.2 Hz, 1H), 3.84-3.82 (m, 4H), 3.38-3.30 (m, 4H), 2.39 (s, 3H), 2.06 (s, 3H), 1.59 (d, J=6.8 Hz, 3H).

### Preparation of 4-(6-chloro-3-((1-(3,6-dimethyl-2-morpholino-4-oxo-4H-chromen-8-yl) ethyl)amino)pyridin-2-yl)-2-fluoro-6-formylphenyl trifluoromethanesulfonate

The title compound was prepared as a yellow solid (2.70 g, 3.95 mmol, 68.09% yield) according to the procedures described herein and the synthetic scheme, using 5-(6-chloro-3-((1-(3,6-dimethyl-2-morpholino-4-oxo-4H-chromen-8-yl)ethyl)amino)pyridin-2-yl)-3-fluoro-2-hydroxybenzaldehyde (3.20 g, 5.80 mmol, 1 eq) and PhNTf₂ (4.14 g, 11.59 mmol, 2 eq). ¹H NMR (CHCl₃, 400 MHz) δ 10.28 (s, 1H), 8.25 (d, J=1.6 Hz, 1H), 8.00 (dd, J=10.0 Hz, J=2.0 Hz, 1H), 7.91 (s, 1H), 7.56 (s, 1H), 7.33 (d, J=2.4 Hz, 1H), 7.06 (d, J=8.8 Hz, 1H), 6.69 (d, J=8.8 Hz, 1H), 4.96-4.91 (m, 1H), 4.49-4.49 (m, 1H), 3.86-3.83 (m, 4H), 3.39-3.36 (m, 4H), 2.39 (s, 3H), 2.07 (s, 3H), 1.62 (d, J=6.8 Hz, 3H).

### Preparation of 5-(6-chloro-3-((1-(3,6-dimethyl-2-morpholino-4-oxo-4H-chromen-8-yl) ethyl)amino)pyridin-2-yl)-3-fluoro-2-(4,4,5,5-tetramethyl-1,3,2-dioxaborolan-2-yl)benzaldehyde

The title compound was prepared as yellow oil (200 mg, crude) according to the procedures described herein and the synthetic scheme, using 4-(6-chloro-3-((1-(3,6-dimethyl-2-morpholino-4-oxo-4H-chromen-8-yl)ethyl)amino)pyridin-2-yl)-2-fluoro-6-formylphenyl trifluoromethanesulfonate (200 mg, 292 μmol, 1 eq) and B₂Pin₂ (371 mg, 1.46 mmol, 5 eq). The product was used directly for next step without further purification.

### Preparation of 8-(1-((6-chloro-2-(7-fluoro-1-hydroxy-1,3-dihydrobenzo[c][1,2]oxaborol-5-yl)pyridin-3-yl)amino)ethyl)-3,6-dimethyl-2-morpholino-4H-chromen-4-one

A mixture of 5-(6-chloro-3-((1-(3,6-dimethyl-2-morpholino-4-oxo-4H-chromen-8-yl)ethyl)amino)pyridine-2-yl)-3-fluoro-2-(4,4,5,5-tetramethyl-1,3,2-dioxaborolan-2-yl)benzaldehyde (200 mg, 302 μmol, 1 eq) and NaBH₃CN (38 mg, 604 μmol, 2 eq) in THF (2 mL) was degassed and purged with N₂ for 3 times at 20° C., and then the mixture was stirred at 20° C. for 1 h under N₂ atmosphere. The reaction mixture was quenched by H₂O (2 mL) at 20° C., then filtered and the filter cake was dried in vacuo to give the title compound (70 mg, 124.16 μmol, 41.09% yield) as a white solid. ¹H NMR (DMSO-d₆, 400 MHz) δ 9.34 (s, 1H), 7.63 (d, J=1.2 Hz, 1H), 7.52 (s, 1H), 7.45 (d, J=2.0 Hz, 1H), 7.35 (d, J=8.8 Hz, 1H), 7.16 (d, J=8.4 Hz, 1H), 6.95 (d, J=8.8 Hz, 1H), 5.75 (d, J=7.2 Hz, 1H), 5.09-4.99 (m, 2H), 4.94-4.91 (m, 1H), 3.71-3.68 (m, 4H), 3.27-3.26 (m, 4H), 2.32 (s, 3H), 1.91 (s, 3H), 1.50 (d, J=6.4 Hz, 3H).

### Preparation of (S)-8-(1-((6-chloro-2-(7-fluoro-1-hydroxy-1,3-dihydrobenzo[c][1,2]oxaborol-5-yl)pyridin-3-yl)amino)ethyl)-3,6-dimethyl-2-morpholino-4H-chromen-4-one and (R)-8-(1-((6-chloro-2-(7-fluoro-1-hydroxy-1,3-dihydrobenzo[c][1,2]oxaborol-5-yl)pyridin-3-yl)amino)ethyl)-3,6-dimethyl-2-morpholino-4H-chromen-4-one

8-(1-((6-chloro-2-(7-fluoro-1-hydroxy-1,3-dihydrobenzo[c][1,2]oxaborol-5-yl)pyridin-3-yl)amino)ethyl)-3,6-dimethyl-2-morpholino-4H-chromen-4-one (120 mg, 212 μmol, 1 eq) was separated by SFC (column: DAICEL CHIRALCEL OD (250 mm×30 mm×10 um); mobile phase: [CO2-IPA]; B %:50%, isocratic elution mode) to give the title compound, Isomer 1 (53 mg, 93.65 μmol, 44.00% yield) as a white solid and the title compound, Isomer 2 (49.2 mg, 86.57 gmol, 40.67% yield) as a white solid. Isomer 1: ¹H NMR (DMSO-d₆, 400 MHz) δ 9.34 (s, 1H), 7.63 (s, 1H), 7.52-7.34 (m, 2H), 7.17-7.09 (m, 2H), 6.96-6.81 (m, 1H), 5.74 (d, J=7.2 Hz, 1H), 5.09-4.94 (m, 2H), 4.74-4.66 (m, 1H), 3.71-3.68 (m, 4H), 3.37-3.36 (m, 4H), 2.32 (s, 3H), 1.91 (s, 3H), 1.50 (d, J=6.8 Hz, 3H). MS (ESI): mass calcd. For C₂₉H₂₈BClFN₃O₅ 563.18, m/z found 564.1 [M+H]⁺. HPLC: 99.62% (220 nm), 99.82% (254 nm). Chiral purity: 100% ee. Isomer 2: ¹H NMR (DMSO-d₆, 400 MHz) δ 9.34 (s, 1H), 7.63 (s, 1H), 7.52-7.45 (m, 2H), 7.17-7.08 (m, 2H), 6.96-6.81 (m, 1H), 5.74 (d, J=7.2 Hz, 1H), 5.09-4.98 (m, 2H), 4.96-4.66 (m, 1H), 3.71-3.68 (m, 4H), 3.37-3.36 (m, 4H), 2.32 (s, 3H), 1.91 (s, 3H), 1.52-1.49 (m, 3H). MS (ESI): mass calcd. For C₂₉H₂₈BClFN₃O₅ 563.18, m/z found 564.1 [M+H]⁺. HPLC: 99.20% (220 nm), 99.27% (254 nm). Chiral purity: 97.42% ee

### Example 102

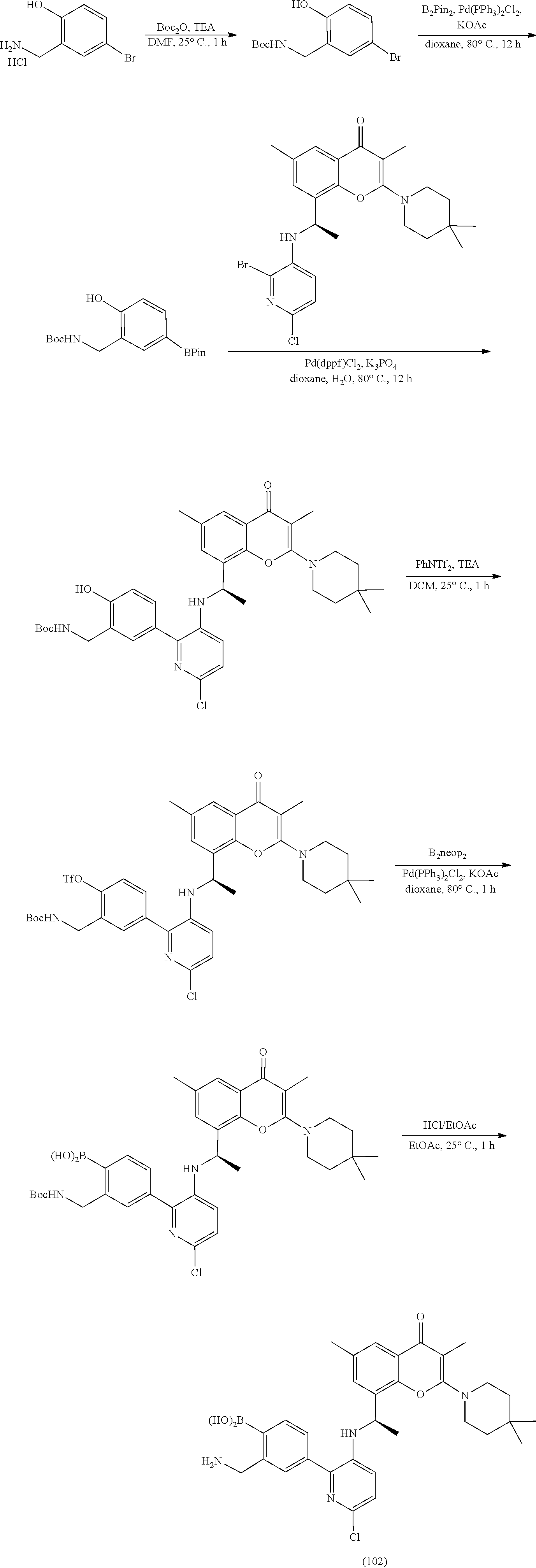

### Preparation of tert-butyl (5-bromo-2-hydroxybenzyl)carbamate

To a solution of 2-(aminomethyl)-4-bromo-phenol (1.50 g, 6.29 mmol, 1 eq, HCl) and TEA (955 mg, 9.43 mmol, 1.31 mL, 1.5 eq) in DMF (14 mL) was added Boc₂O (1.37 g, 6.29 mmol, 1.44 mL, 1 eq) dropwise at 0° C., the reaction mixture was warmed to 25° C. and stirred at 25° C. for 1 h. The reaction mixture was quenched with H₂O (20 mL) at 25° C. and extracted with DCM (15 mL×2). The combined organic layers were washed with brine (15 mL×3) and concentrated under reduced pressure. The residue was purified by flash silica gel chromatography (Biotage®; 40 g SepaFlash® Silica Flash Column, Eluent of 0~10% Ethyl acetate/Petroleum ether gradient @30 mL/min) to give the title compound (1.70 g, 5.63 mmol, 89.46% yield) as a white solid. ¹H NMR (DMSO-d₆, 400 MHz) δ 9.09 (s, 1H), 7.31-7.27 (m, 1H), 7.18 (d, J=2.4 Hz, 1H), 6.84 (d, J=8.4 Hz, 1H), 5.24 (s, 1H), 4.18 (d, J=6.4 Hz, 2H), 1.45 (s, 9H).

### Preparation of tert-butyl (2-hydroxy-5-(4,4,5,5-tetramethyl-1,3,2-dioxaborolan-2-yl)benzyl)carbamate

A mixture of tert-butyl N-[(5-bromo-2-hydroxy-phenyl)methyl]carbamate (1.65 g, 5.46 mmol, 1 eq), B₂Pin₂ (4.16 g, 16.38 mmol, 3 eq), Pd(PPh₃)₂Cl₂ (383.28 mg, 546.06 μmol, 0.1 eq) and KOAc (1.07 g, 10.92 mmol, 2 eq) in dioxane (20 mL) was degassed and purged with N₂ for 3 times at 25° C., and then the reaction mixture was heated to 80° C. and stirred at 80° C. for 12 h under N₂ atmosphere. The reaction mixture was cooled to 25° C., filtered and the filtrate was concentrated under reduced pressure. The residue was purified by flash silica gel chromatography (Biotage®; 40 g SepaFlash® Silica Flash Column, Eluent of 0~10% Ethyl acetate/Petroleum ether gradient @30 mL/min) and then triturated with PE (30 mL) to give the title compound (1.3 g, 3.72 mmol, 68.17% yield) as a white solid. ¹H NMR (DMSO-d₆, 400 MHz) δ 9.27 (s, 1H), 7.69-7.67 (m, 1H), 7.54 (d, J=1.6 Hz, 1H), 6.95 (d, J=8.0 Hz, 1H), 5.21-5.19 (m, 1H), 4.24 (d, J=6.8 Hz, 2H), 1.44 (s, 9H), 1.33 (s, 12H).

### Preparation of tert-butyl (R)-(5-(6-chloro-3-((1-(2-(4,4-dimethylpiperidin-1-yl)-3,6-dimethyl-4-oxo-4H-chromen-8-yl)ethyl)amino)pyridin-2-yl)-2-hydroxybenzyl)carbamate

A mixture of tert-butyl N-[[2-hydroxy-5-(4,4,5,5-tetramethyl-1,3,2-dioxaborolan-2-yl)phenyl]methyl]carbamate (673 mg, 1.93 mmol, 1 eq), 8-[(1R)-1-[(2-bromo-6-chloro-3-pyridyl)amino]ethyl]-2-(4,4-dimethyl-1-piperidyl)-3,6-dimethyl-chromen-4-one (1.00 g, 1.93 mmol, 1 eq), K₃PO₄ (1.23 g, 5.78 mmol, 3 eq), and Pd(dppf)Cl₂ (141 mg, 192.73 μmol, 0.1 eq) in dioxane (5 mL)/H₂O (0.5 mL) was degassed and purged with N₂ for 3 times at 25° C., and then the reaction mixture was heated to 80° C. and stirred at 80° C. for 2 h under N₂ atmosphere. The reaction mixture was cooled to room temperature, filtered and the filtrate was concentrated under reduced pressure. The residue was purified by flash silica gel chromatography (Biotage®; 40 g SepaFlash® Silica Flash Column, Eluent of 0-48% Ethyl acetate/Petroleum ether gradient @30 mL/min) to give the title compound (1.30 g, crude) as a white solid, which was used directly for next step without further purification. ¹H NMR (DMSO-d₆, 400 MHz) δ 9.40 (s, 1H), 7.90 (s, 1H), 7.60-7.57 (m, 1H), 7.39 (d, J=2.0 Hz, 1H), 7.06 (d, J=8.4 Hz, 1H), 6.95 (d, J=8.4 Hz, 1H), 6.59 (d, J=8.8 Hz, 1H), 5.34-5.31 (m, 1H), 4.89-4.85 (m, 1H), 4.60-4.55 (m, 1H), 4.30-4.27 (m, 2H), 3.37-3.34 (m, 4H), 2.38 (s, 3H), 2.07 (s, 3H), 2.06 (s, 3H), 1.55 (d, J=6.4 Hz, 3H), 1.47 (s, 9H), 1.29-1.27 (m, 2H), 1.03 (s, 6H).

### Preparation of (R)-2-(((tert-butoxycarbonyl)amino)methyl)-4-(6-chloro-3-((1-(2-(4,4-dimethylpiperidin-1-yl)-3,6-dimethyl-4-oxo-4H-chromen-8-yl)ethyl)amino)pyridin-2-yl)phenyl trifluoromethanesulfonate

To a solution of tert-butyl N-[[5-[6-chloro-3-[[(1R)-1-[2-(4,4-dimethyl-1-piperidyl)-3,6-dimethyl-4-oxo-chromen-8-yl]ethyl]amino]-2-pyridyl]-2-hydroxy-phenyl]methyl]carbamate (1.00 g, 1.51 mmol, 1 eq) and TEA (306 mg, 3.02 mmol, 421.00 μL, 2 eq) in DCM (10 mL) was added 1,1,1-trifluoro-N-phenyl-N-(trifluoromethylsulfonyl) methanesulfonamide (1.08 g, 3.02 mmol, 2 eq) and DMAP (18.48 mg, 151.23 μmol, 0.1 eq) in turns at 25° C., the reaction mixture was stirred at 25° C. for 1 h. The reaction mixture was quenched with H₂O (10 mL) at 25° C. and extratcted with DCM (10 mL×2). The combined organic layers were washed with brine (10 mL) and concentrated under reduced pressure. The residue was purified by flash silica gel chromatography (Biotage®; 40 g SepaFlash® Silica Flash Column, Eluent of 0-38% Ethyl acetate/Petroleum ether gradient @30 m/min) to give the title compound (1.00 g, 1.26 mmol, 83.35% yield) as a yellow solid.

### Preparation of (R)-(2-(((tert-butoxycarbonyl)amino)methyl)-4-(6-chloro-3-((1-(2-(4,4-dimethylpiperidin-1-yl)-3,6-dimethyl-4-oxo-4H-chromen-8-yl)ethyl)amino)pyridin-2-yl)phenyl)boronic acid

The title compound was prepared as a white solid (200 mg, 264.14 μmol, 69.85% yield) according to the procedures described herein and the synthetic scheme, using [2-[(tert-butoxycarbonylamino)methyl]-4-[6-chloro-3-[[(1R)-1-[2-(4,4-dimethyl-1-piperidyl)-3,6-dimethyl-4-oxo-chromen-8-yl]ethyl]amino]-2-pyridyl]phenyl]trifluoromethanesulfonate (300 mg, 378.17 μmol, 1 eq) and 2-(5,5-dimethyl-1,3,2-dioxaborinan-2-yl)-5,5-dimethyl-1,3,2-dioxaborinane (427 mg, 1.89 mmol, 5 eq). The product was used directly for next step without further purification.

### Preparation of [2-(aminomethyl)-4-[6-chloro-3-[[(1R)-1-[2-(4,4-dimethyl-1-piperidyl)-3,6-dimethyl-4-oxo-chromen-8-yl]ethyl]amino]-2-pyridyl]phenyl]boronic acid

To a solution of (R)-(2-(((tert-butoxycarbonyl)amino)methyl)-4-(6-chloro-3-((1-(2-(4,4-dimethylpiperidin-1-yl)-3,6-dimethyl-4-oxo-4H-chromen-8-yl)ethyl)amino)pyridin-2-yl)phenyl)boronic acid (54.94 mg, 72.56 μmol, 1 eq) in EtOAc (1 mL) was added HCl/EtOAc (4 N, 36.28 μL, 2 eq) in one portion at 25° C., the reaction mixture was stirred at 25° C. for 1 h. The reaction mixture was diluted with H₂O (5 mL), filtered and the filter cake was dried in vacuo. Then the crude product was further triturated with H₂O (2 mL) at 25° C. for 10 min to give the title compound (25 mg, 42.45 μmol, 58.50% yield) as a white solid. ¹H NMR (DMSO-d₆, 400 MHz) δ 7.60 (s, 1H), 7.90 (s, 1H), 7.42-7.36 (m, 3H), 7.08 (d, J=8.8 Hz, 1H), 6.92-6.90 (m, 1H), 4.91-4.89 (m, 1H), 4.10 (s, 3H), 3.27 (s, 4H), 2.27 (s, 3H), 1.86 (s, 3H), 1.46 (d, J=6 Hz, 3H), 1.33 (m, 4H), 0.89 (s, 6H). MS (ESI): mass calcd. For C₃₂H₃₈BClN₄O₄ 588.27, m/z found 587.1 [M−H]. HPLC: 97.55% (220 nm), 98.34% (254 nm). Chiral purity: 99.58% ee

### Example 103

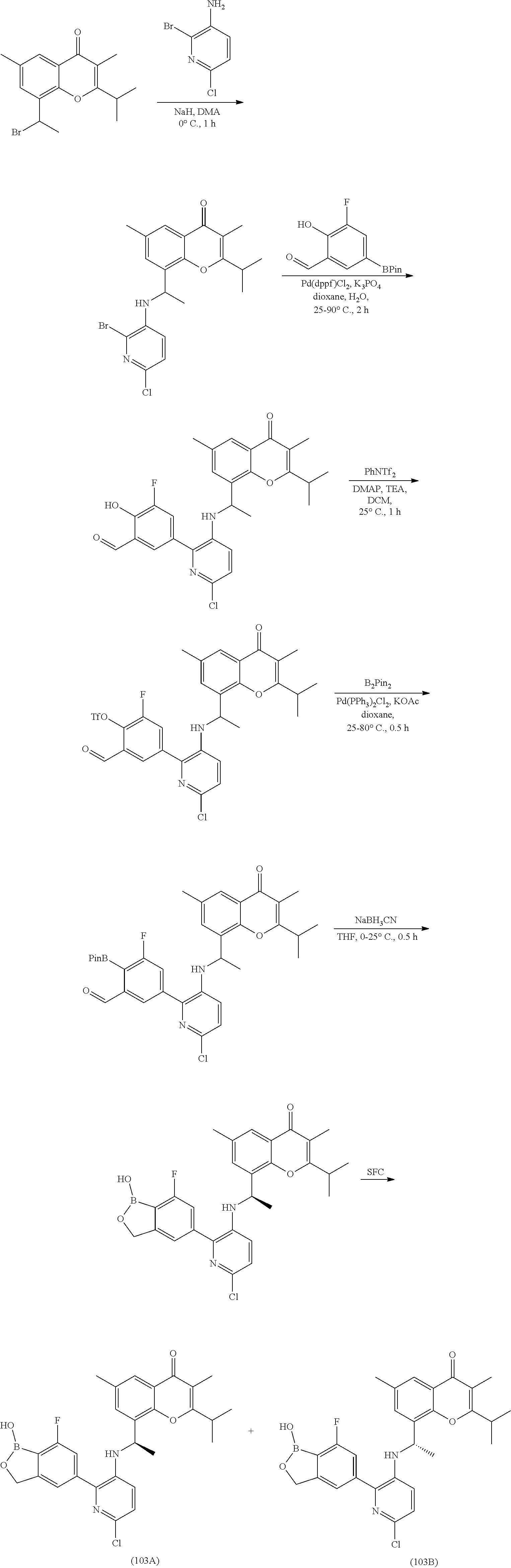

### Preparation of 8-(1-((2-bromo-6-chloropyridin-3-yl)amino)ethyl)-2-isopropyl-3,6-dimethyl-4H-chromen-4-one

The title compound was prepared as a white solid (4.40 g, 9.78 mmol, 79.05% yield) according to the procedures described herein and the synthetic scheme, using 8-(1-bromoethyl)-2-isopropyl-3,6-dimethyl-chromen-4-one (4.00 g, 12.38 mmol, 1 eq) and 2-bromo-6-chloro-pyridin-3-amine (2.82 g, 13.61 mmol, 1.1 eq). ¹H NMR ((DMSO-d₆, 400 MHz) δ 7.70 (s, 1H), 7.53 (s, 1H), 7.20 (d, J=8.4 Hz, 1H), 6.74 (d, J=8.8 Hz, 1H), 5.82 (d, J=6.8 Hz, 1H), 5.07 (t, J=7.2 Hz, 1H), 2.34 (s, 3H), 2.00 (s, 3H), 1.64 (d, J=6.8 Hz, 3H), 1.30 (dd, J=6.8 Hz, 2.4 Hz, 6H).

### Preparation of 5-(6-chloro-3-((1-(2-isopropyl-3,6-dimethyl-4-oxo-4H-chromen-8-yl) ethyl)amino)pyridin-2-yl)-3-fluoro-2-hydroxybenzaldehyde

The title compound was prepared as a brown solid (3.00 g, 5.89 mmol, 61.65% yield) according to the procedures described herein and the synthetic scheme, using 8-[1-[(2-bromo-6-chloro-3-pyridyl)amino]ethyl]-2-isopropyl-3,6-dimethyl-chromen-4-one (4.30 g, 9.56 mmol, 1 eq) and 3-fluoro-2-hydroxy-5-(4,4,5,5-tetramethyl-1,3,2-dioxaborolan-2-yl) benzaldehyde (3.82 g, 14.34 mmol, 1.5 eq). ¹H NMR (DMSO-d₆, 400 MHz) δ 11.23 (s, 1H), 10.34 (s, 1H), 7.94 (s, 1H), 7.89 (s, 1H), 7.68 (s, 1H), 7.56 (s, 1H), 7.13 (d, J=8.4 Hz, 1H), 6.84 (d, J=8.8 Hz, 1H), 5.89 (d, J=7.6 Hz, 1H), 3.30-3.28 (m, 1H), 2.34 (s, 3H), 1.51 (d, J=6.4 Hz, 3H), 1.26 (d, J=6.8 Hz, 3H), 1.20 (d, J=6.0 Hz, 3H), 1.16 (d, J=6.8 Hz, 3H).

### Preparation of 4-(6-chloro-3-((1-(2-isopropyl-3,6-dimethyl-4-oxo-4H-chromen-8-yl) ethyl)amino)pyridin-2-yl)-2-fluoro-6-formylphenyl trifluoromethanesulfonate

The title compound was prepared as a yellow solid (2.30 g, 3.59 mmol, 65.22% yield) according to the procedure described in preceding examples, using 5-[6-chloro-3-[1-(2-isopropyl-3,6-dimethyl-4-oxo-chromen-8-yl)ethylamino]-2-pyridyl]-3-fluoro-2-hydroxy-benzaldehyde (2.80 g, 5.50 mmol, 1 eq) and 1,1,1-trifluoro-N-phenyl-N-(trifluoromethylsulfonyl)methanesulfonamide (2.95 g, 8.25 mmol, 1.5 eq). ¹H NMR (DMSO-d₆, 400 MHz) δ 10.13 (s, 1H), 8.34-8.31 (m, 2H), 7.70 (s, 1H), 7.56 (s, 1H), 7.43-7.39 (m, 1H), 6.86 (d, J=8.8 Hz, 1H), 6.32 (d, J=6.8 Hz, 1H), 5.01 (t, J=6.8 Hz, 1H), 3.28-3.26 (m, 1H), 2.34 (s, 1H), 1.98 (s, 3H), 1.54 (d, J=6.8 Hz, 3H), 1.29 (d, J=6.8 Hz, 3H), 1.25 (d, J=6.8 Hz, 3H).

### Preparation of 5-(6-chloro-3-((1-(2-isopropyl-3,6-dimethyl-4-oxo-4H-chromen-8-yl) ethyl)amino)pyridin-2-yl)-3-fluoro-2-(4,4,5,5-tetramethyl-1,3,2-dioxaborolan-2-yl)benzaldehyde

The title compound was prepared as brown oil (5.00 g, crude) according to the procedures described herein and the synthetic scheme, using [4-[6-chloro-3-[1-(2-isopropyl-3,6-dimethyl-4-oxo-chromen-8-yl)ethylamino]-2-pyridyl]-2-fluoro-6-formyl-phenyl]trifluoromethanesulfonate (2.00 g, 3.12 mmol, 1 eq) and B₂Pin₂ (2.38 g, 9.36 mmol, 3 eq). ¹H NMR (DMSO-d₆, 400 MHz) δ 10.04 (s, 1H), 8.17 (s, 1H), 7.68 (d, J=6.8 Hz, 1H), 7.20 (d, J=8.8 Hz, 1H), 6.87 (d, J=8.8 Hz, 1H), 6.07 (d, J=7.2 Hz, 1H), 5.00 (t, J=6.8 Hz, 1H), 3.31-3.29 (m, 1H), 2.34 (s, 1H), 1.91 (s, 1H), 1.52 (d, J=8.8 Hz, 3H), 1.28 (d, J=12.0 Hz, 3H), 1.23 (d, J=12.0 Hz, 3H), 1.01 (s, 12H).

### Preparation of 8-(1-((6-chloro-2-(7-fluoro-1-hydroxy-1,3-dihydrobenzo[c][1,2]oxaborol-5-yl)pyridin-3-yl)amino)ethyl)-2-isopropyl-3,6-dimethyl-4H-chromen-4-one

To a solution of 5-[6-chloro-3-[1-(2-isopropyl-3,6-dimethyl-4-oxo-chromen-8-yl)ethylamino]-2-pyridyl]-3-fluoro-2-(4,4,5,5-tetramethyl-1,3,2-dioxaborolan-2-yl)benzaldehyde (4.00 g, 3.23 mmol, 1 eq) in THF (40 mL) was added NaBH₃CN (406 mg, 6.46 mmol, 2 eq) in portions slowly at 0° C., the reaction mixture was warmed up to 25° C. and stirred at 25° C. for 0.5 h. The reaction mixture was quenched by H₂O (10 mL) and then extracted with EtOAc (15 mL×3). The combined organic layers were washed with brine (20 mL×2), dried over Na₂SO₄, filtered and concentrated under reduced pressure to give a residue. The residue was purified by flash silica gel chromatography (Biotage®; 40 g SepaFlash® Silica Flash Column, Eluent of 0~50% Ethyl acetate/Petroleum ether gradient @60 mL/min) to give the title compound (400.0 mg, 734.15 μmol, 22.72% yield) as a brown solid.

### Preparation of 8-[(1S)-1-[[6-chloro-2-(7-fluoro-1-hydroxy-3H-2,1-benzoxaborol-5-yl)-3-pyridyl]amino]ethyl]-2-isopropyl-3,6-dimethyl-chromen-4-one and 8-[(1R)-1-[[6-chloro-2-(7-fluoro-1-hydroxy-3H-2,1-benzoxaborol-5-yl)-3-pyridyl]amino]ethyl]-2-isopropyl-3,6-dimethyl-chromen-4-one

8-[1-[[6-chloro-2-(7-fluoro-1-hydroxy-3H-2,1-benzoxaborol-5-yl)-3-pyridyl]amino]ethyl]-2-isopropyl-3,6-dimethyl-chromen-4-one (400 mg, 1 eq) was separated by SFC (column: DAICEL CHIRALPAK IG (250 mm×30 mm×10 um); mobile phase: [CO₂—IPA]; B %:45%, isocratic elution mode) to give the title compound, Isomer 1 (89.50 mg, 168.81 μmol, 21.98% yield) as a white solid and the title compound, Isomer 2 (90.00 mg, 168.37 μmol, 21.92% yield) as a white solid. Isomer 1: ¹H NMR (DMSO-d₆, 400 MHz) δ 9.37 (s, 1H), 7.68 (s, 1H), 7.56 (s, 2H), 7.38 (d, J=8.8 Hz, 1H), 7.17 (d, J=8.8 Hz, 1H), 6.87 (d, J=8.8 Hz, 1H), 5.80 (d, J=7.2 Hz, 1H), 5.06 (d, J=2.4 Hz, 2H), 5.00 (t, J=6.8 Hz, 1H), 3.30-3.28 (m, 1H), 2.34 (s, 3H), 1.99 (s, 3H), 1.52 (d, J=6.4 Hz, 3H), 1.25 (d, J=6.8 Hz, 3H), 1.19 (d, J=6.4 Hz, 3H). MS (ESI): mass calcd. For C₂₈H₂₇BClFN₂O₄ 520.17, m/z found 521.1 [M+H]⁺. HPLC: 98.23% (220 nm), 98.87% (254 nm). Chiral purity: 100% ee. Isomer 2: ¹H NMR (DMSO-d₆, 400 MHz) δ 9.37 (s, 1H), 7.68 (s, 1H), 7.56 (s, 2H), 7.38 (d, J=8.8 Hz, 1H), 7.17 (d, J=8.8 Hz, 1H), 6.87 (d, J=8.8 Hz, 1H), 5.80 (d, J=7.2 Hz, 1H), 5.06 (d, J=2.4 Hz, 2H), 5.00 (t, J=6.8 Hz, 1H), 3.31-3.29 (m, 1H), 2.34 (s, 3H), 1.99 (s, 3H), 1.52 (d, J=6.4 Hz, 3H), 1.25 (d, J=6.8 Hz, 3H), 1.19 (d, J=6.4 Hz, 3H). MS (ESI): mass calcd. For C₂₈H₇BClFN₂O₄ 520.17, m/z found 521.1 [M+H]⁺. HPLC: 97.43% (220 nm), 98.95% (254 nm). Chiral purity: 99.48% ee

### Example 104

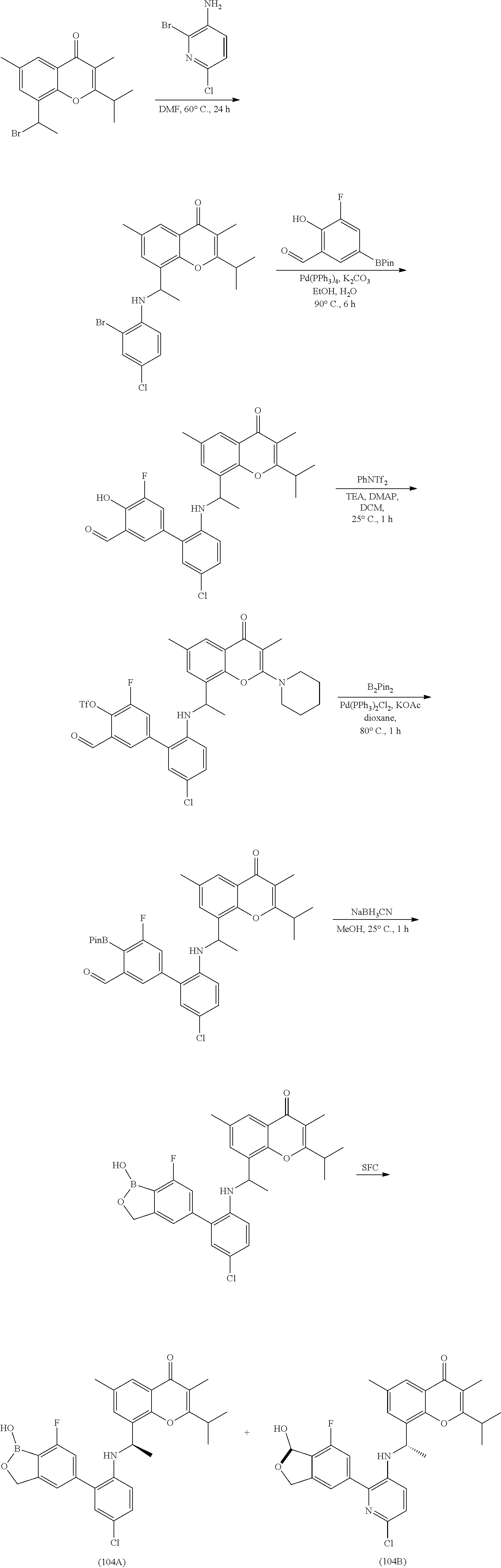

### Preparation of 8-[1-(2-bromo-4-chloro-anilino)ethyl]-2-isopropyl-3,6-dimethyl-chromen-4-one

The title compound was prepared as a brown solid (1.50 g, 3.34 mmol, 90.03% yield) according to the procedures described herein and the synthetic scheme, using 8-(1-bromoethyl)-2-isopropyl-3,6-dimethyl-chromen-4-one (1.20 g, 3.71 mmol, 1 eq) and 2-bromo-4-chloro-aniline (920 mg, 4.46 mmol, 1.2 eq). ¹H NMR (DMSO-d₆, 400 MHz) δ 7.70 (s, 1H), 7.40 (d, J=2.4 Hz, 1H), 7.11 (t, J=2.4 Hz, 1H), 6.78 (d, J=8.8 Hz, 1H), 6.36 (d, J=9.2 Hz, 1H), 5.52 (d, J=6.8 Hz, 1H), 5.09-5.06 (m, 1H), 2.42 (d, J=4.0 Hz, 1H), 2.33 (s, 3H), 2.00 (s, 3H), 1.62 (d, J=6.4 Hz, 3H), 1.33-1.29 (m, 6H).

### Preparation of 5-[5-chloro-2-[1-(2-isopropyl-3,6-dimethyl-4-oxo-chromen-8-yl) ethylamino]phenyl]-3-fluoro-2-hydroxy-benzaldehyde

The title compound was prepared as a yellow solid (1.10 g, 2.17 mmol, 38.8% yield) according to the procedures described herein and the synthetic scheme, using 8-[1-(2-bromo-4-chloro-anilino)ethyl]-2-isopropyl-3,6-dimethyl-chromen-4-one (2.50 g, 5.57 mmol, 1 eq) and 3-fluoro-2-hydroxy-5-(4,4,5,5-tetramethyl-1,3,2-dioxaborolan-2-yl)benzaldehyde (2.96 g, 11.1 mmol, 2 eq). ¹H NMR (DMSO-d₆, 400 MHz) δ 11.07 (s, 1H), 10.31 (s, 1H), 7.70-7.65 (m, 2H), 7.59 (d, J=1.6 Hz, 1H), 7.52 (d, J=2.0 Hz, 1H), 7.10-7.03 (m, 2H), 6.41 (d, J=8.8 Hz, 1H), 5.31 (d, J=7.2 Hz, 1H), 5.02-4.95 (m, 1H), 3.31-3.27 (m, 1H), 2.34 (s, 3H), 2.03-1.95 (m, 3H), 1.46 (d, J=6.4 Hz, 3H), 1.28 (d, J=6.8 Hz, 3H), 1.21 (d, J=6.8 Hz, 3H).

### Preparation of [4-[5-chloro-2-[1-(2-isopropyl-3,6-dimethyl-4-oxo-chromen-8-yl)ethyl amino]phenyl]-2-fluoro-6-formyl-phenyl]trifluoromethanesulfonate

The title compound was prepared as yellow oil (1.20 g, 1.87 mmol, 86.5% yield) according to the procedures described herein and the synthetic scheme, using 5-[5-chloro-2-[1-(2-isopropyl-3,6-dimethyl-4-oxo-chromen-8-yl)ethylamino]phenyl]-3-fluoro-2-hydroxy-benzaldehyde (1.10 g, 2.17 mmol, 1 eq) and 1,1,1-trifluoro-N-phenyl-N-(trifluoromethylsulfonyl)methanesulfonamide (1.01 g, 2.82 mmol, 1.3 eq). ¹H NMR (DMSO-d₆, 400 MHz) δ 10.10 (d, J=1.6 Hz, 1H), 7.68 (s, 1H), 7.55 (d, J=2.0 Hz, 1H), 7.45-7.36 (m, 1H), 7.31-7.22 (m, 2H), 7.16 (dd, J=2.4, 8.8 Hz, 1H), 6.42 (d, J=8.8 Hz, 1H), 5.75 (d, J=7.2 Hz, 1H), 5.06-4.93 (m, 1H), 3.33-3.28 (m, 1H), 2.34 (s, 3H), 1.99 (s, 3H), 1.48 (d, J=6.4 Hz, 3H), 1.30 (d, J=6.8 Hz, 3H), 1.24 (d, J=6.8 Hz, 3H).

### Preparation of 5-[5-chloro-2-[1-(2-isopropyl-3,6-dimethyl-4-oxo-chromen-8-yl) ethylamino]phenyl]-3-fluoro-2-(4,4,5,5-tetramethyl-1,3,2-dioxaborolan-2-yl)benzaldehyde

The title compound was prepared as a light yellow solid (360 mg, crude) according to the procedures described herein and the synthetic scheme, using [4-[5-chloro-2-[1-(2-isopropyl-3,6-dimethyl-4-oxo-chromen-8-yl)ethyl amino]phenyl]-2-fluoro-6-formyl-phenyl]trifluoromethanesulfonate (650 mg, 1.02 mmol, 1 eq) and B₂Pin₂ (1.27 g, 5.00 mmol, 4 eq). The product was used directly for next step without further purification.

### Preparation of 8-[1-[4-chloro-2-(7-fluoro-1-hydroxy-3H-2,1-benzoxaborol-5-yl)anilino]ethyl]-2-isopropyl-3,6-dimethyl-chromen-4-one

To a solution of 5-[5-chloro-2-[1-(2-isopropyl-3,6-dimethyl-4-oxo-chromen-8-yl)ethyl amino]phenyl]-3-fluoro-2-(4,4,5,5-tetramethyl-1,3,2-dioxaborolan-2-yl)benzaldehyde (1.70 g, 1.38 mmol, 1 eq) in MeOH (30 mL) was added NaBH₃CN (130 mg, 2.06 mmol, 1.5 eq) in portions at 25° C., the reaction mixture was stirred at 25° C. for 1 h. The reaction mixture was quenched by adding water (40 mL), and extracted with EtOAc (30 mL×3). The combined organic layers were washed with brine (50 mL), dried over Na₂SO₄, filtered and concentrated under reduced pressure to give a residue. The residue was purified by prep-HPLC (column: Waters Xbridge Prep OBD C18 150×40 mm×10 um; mobile phase: [H2O (10 mM NH4HCO3)-AC□]; gradient: 55%-90% B over 8.0 min) to give the title compound (250 mg, 480.96 μmol, 34.96% yield) as a white solid. ¹H NMR (DMSO-d₆, 400 MHz) δ 9.33 (s, 1H), 7.68 (s, 1H), 7.53 (d, J=2.0 Hz, 1H), 7.36 (s, 1H), 7.20 (d, J=9.2 Hz, 1H), 7.16-7.00 (m, 2H), 6.44 (d, J=8.8 Hz, 1H), 5.22 (d, J=7.6 Hz, 1H), 5.10-4.92 (m, 3H), 3.30-3.26 (m, 1H), 2.34 (s, 3H), 1.99 (s, 3H), 1.47 (d, J=6.8 Hz, 3H), 1.26 (d, J=6.8 Hz, 3H), 1.21-1.16 (m, 3H).

### Preparation of (S)-8-(1-((4-chloro-2-(7-fluoro-1-hydroxy-1,3-dihydrobenzo[c][1,2]oxaborol-5-yl)phenyl)amino)ethyl)-2-isopropyl-3,6-dimethyl-4H-chromen-4-one and (R)-8-(1-((4-chloro-2-(7-fluoro-1-hydroxy-1,3-dihydrobenzo[c][1,2]oxaborol-5-yl)phenyl)amino)ethyl)-2-isopropyl-3,6-dimethyl-4H-chromen-4-one

8-[1-[4-chloro-2-(7-fluoro-1-hydroxy-3H-2,1-benzoxaborol-5-yl)anilino]ethyl]-2-isopropyl-3,6-dimethyl-chromen-4-one (0.25 g, 480 μmol, 1 eq) was separated by SFC (column: DAICEL CHIRALPAK AD (250 mm×30 mm, 10 um); mobile phase: [CO2-IPA]; B %:30%, isocratic elution mode) to give the title compound, Isomer 1 (115.2 mg, 221.62 μmol, 46.08% yield) as a white solid and the title compound, Isomer 2 (107.2 mg, 206.23 μmol, 42.88% yield) as a white solid. Isomer 1: ¹H NMR (DMSO-d₆, 400 MHz) δ 9.34 (s, 1H), 7.68 (d, J=1.2 Hz, 1H), 7.53 (d, J=2.0 Hz, 1H), 7.36 (s, 1H), 7.20 (d, J=8.8 Hz, 1H), 7.10 (dd, J=2.4, 8.4 Hz, 1H), 7.06 (d, J=2.8 Hz, 1H), 6.44 (d, J=8.8 Hz, 1H), 5.23 (d, J=6.8 Hz, 1H), 5.04 (s, 2H), 5.01-4.95 (m, 1H), 3.31-3.23 (m, 1H), 2.34 (s, 3H), 1.99 (s, 3H), 1.47 (d, J=6.4 Hz, 3H), 1.26 (d, J=6.8 Hz, 3H), 1.18 (d, J=6.8 Hz, 3H). MS (ESI): mass calcd. For C₂₉H₂₈BClFNO₄ 519.18, m/z found 520.0 [M+H]⁺. HPLC: 98.96% (220 nm), 99.21% (254 nm). Chiral purity: 99.80% ee. Isomer 2: ¹H NMR (DMSO-d₆, 400 MHz) δ 9.34 (s, 1H), 7.68 (d, J=1.2 Hz, 1H), 7.53 (d, J=2.0 Hz, 1H), 7.36 (s, 1H), 7.20 (d, J=8.8 Hz, 1H), 7.10 (dd, J=2.4, 8.8 Hz, 1H), 7.06 (d, J=2.4 Hz, 1H), 6.44 (d, J=8.8 Hz, 1H), 5.23 (d, J=7.2 Hz, 1H), 5.04 (d, J=1.6 Hz, 2H), 5.01-4.95 (m, 1H), 3.31-3.25 (m, 1H), 2.34 (s, 3H), 1.99 (s, 3H), 1.47 (d, J=6.4 Hz, 3H), 1.26 (d, J=6.8 Hz, 3H), 1.20-1.15 (m, 3H). MS (ESI): mass calcd. For C₂₉H₂₈BClFNO₄ 519.18, m/z found 520.0 [M+H]⁺. HPLC: 98.35% (220 nm), 98.35% (254 nm). Chiral purity: 99.28% ee

### Example 105

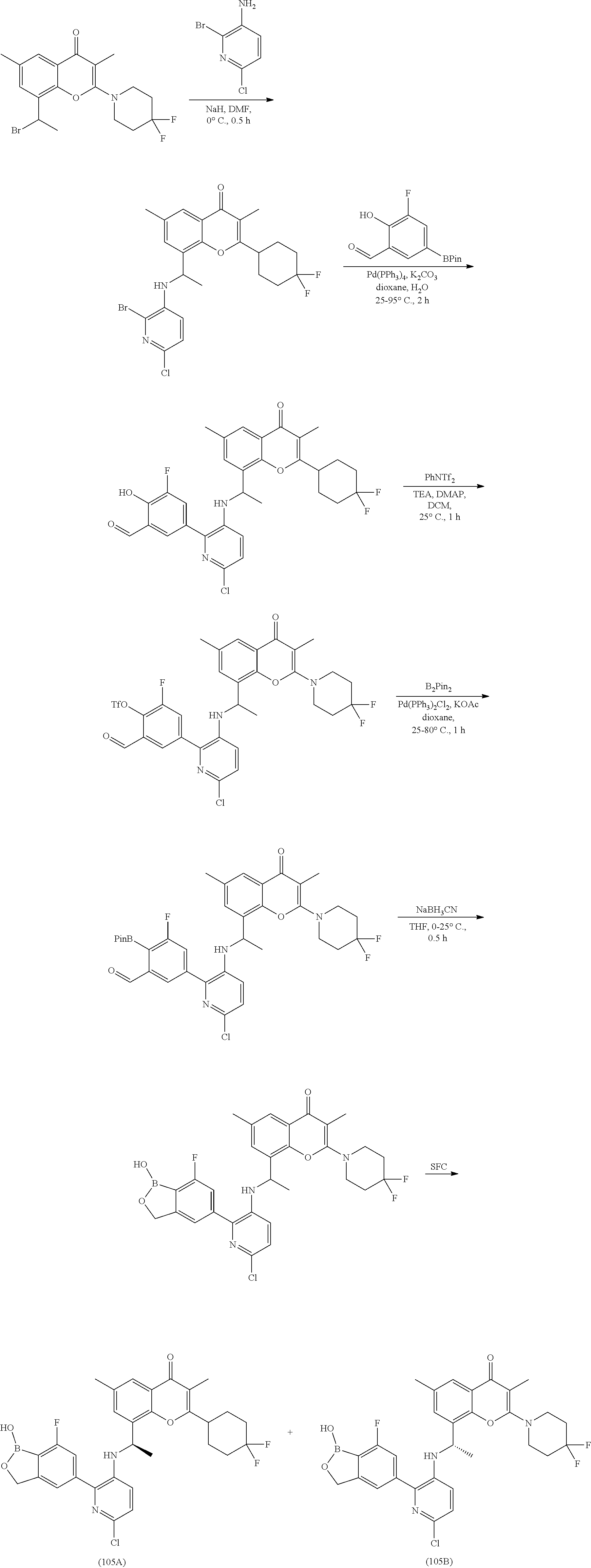

### Preparation of 8-(1-((2-bromo-6-chloropyridin-3-yl)amino)ethyl)-2-(4,4-difluoro piperidin-1-yl)-3,6-dimethyl-4H-chromen-4-one

The title compound was prepared as a yellow solid (950.00 mg, 1.80 mmol, 55.52% yield) according to the procedures described herein and the synthetic scheme, using 8-(1-bromoethyl)-2-(4,4-difluoropiperidin-1-yl)-3,6-dimethyl-4H-chromen-4-one (1.30 g, 3.25 mmol, 1 eq) and 2-bromo-6-chloropyridin-3-amine (741.2 mg, 3.57 mmol, 1.1 eq). ¹H NMR (DMSO-d₆, 400 MHz) δ 7.65 (s, 1H), 7.42 (s, 1H), 7.17 (d, J=8.4 Hz, 1H), 6.80 (d, J=8.4 Hz, 1H), 5.78 (d, J=6.8 Hz, 1H), 5.02 (t, J=6.8 Hz, 1H), 3.52 (t, J=5.6 Hz, 4H), 2.32 (s, 1H), 2.21-2.12 (m, 4H), 1.95 (s, 3H), 1.63 (d, J=6.8 Hz, 3H).

### Preparation of 5-(6-chloro-3-((1-(2-(4,4-difluoropiperidin-1-yl)-3,6-dimethyl-4-oxo-4H-chromen-8-yl)ethyl)amino)pyridin-2-yl)-3-fluoro-2-hydroxybenzaldehyde

The title compound was prepared as a brown solid (900 mg, crude) according to the procedures described herein and the synthetic scheme, using 8-(1-((2-bromo-6-chloropyridin-3-yl)amino)ethyl)-2-(4,4-difluoropiperidin-1-yl)-3,6-dimethyl-4H-chromen-4-one (900 mg, 1.71 mmol, 1 eq) and 3-fluoro-2-hydroxy-5-(4,4,5,5-tetramethyl-1,3,2-dioxaborolan-2-yl)benzaldehyde (682 mg, 2.56 mmol, 1.5 eq). The product was used directly for next step without further purification.

### Preparation of 4-(6-chloro-3-((1-(2-(4,4-difluoropiperidin-1-yl)-3,6-dimethyl-4-oxo-4H-chromen-8-yl)ethyl)amino)pyridin-2-yl)-2-fluoro-6-formylphenyl trifluoromethanesulfonate

The title compound was prepared as a yellow solid (650.00 mg, 905.21 μmol, 62.41% yield) according to the procedures described herein and the synthetic scheme, using 5-(6-chloro-3-((1-(2-(4,4-difluoropiperidin-1-yl)-3,6-dimethyl-4-oxo-4H-chromen-8-yl)ethyl)amino)pyridin-2-yl)-3-fluoro-2-hydroxybenzaldehyde (850 mg, 1.45 mmol, 1 eq) and 1,1,1-trifluoro-N-phenyl-N-(trifluoromethylsulfonyl)methanesulfonamide (777 mg, 2.18 mmol, 1.5 eq). ¹H NMR (DMSO-d₆, 400 MHz) δ 10.14 (s, 1H), 8.34-8.29 (m, 2H), 7.64 (s, 1H), 7.45 (s, 1H), 7.19 (d, J=8.8 Hz, 1H), 6.92 (d, J=8.8 Hz, 1H), 6.30 (d, J=6.8 Hz, 1H), 4.96 (t, J=6.8 Hz, 1H), 3.52 (t, J=5.2 Hz, 4H), 2.32 (s, 3H), 2.19-2.15 (m, 4H), 1.95 (s, 3H), 1.54 (d, J=6.4 Hz, 3H).

### Preparation of 5-(6-chloro-3-((1-(2-(4,4-difluoropiperidin-1-yl)-3,6-dimethyl-4-oxo-4H-chromen-8-yl)ethyl)amino)pyridin-2-yl)-3-fluoro-2-(4,4,5,5-tetramethyl-1,3,2-dioxaborolan-2-yl)benzaldehyde

The title compound was prepared as brown oil (1.10 g, crude) according to the procedures described herein and the synthetic scheme, using 4-(6-chloro-3-((1-(2-(4,4-difluoropiperidin-1-yl)-3,6-dimethyl-4-oxo-4H-chromen-8-yl)ethyl)amino)pyridin-2-yl)-2-fluoro-6-formylphenyl trifluoromethanesulfonate (600 mg, 835.58 μmol, 1 eq) and B₂Pin₂ (637 mg, 2.51 mmol, 3 eq). The product was used directly for next step without further purification.

### Preparation of 8-(1-((6-chloro-2-(7-fluoro-1-hydroxy-1,3-dihydrobenzo[c][1,2]oxaborol-5-yl)pyridin-3-yl)amino)ethyl)-2-(4,4-difluoropiperidin-1-yl)-3,6-dimethyl-4H-chromen-4-one

To a solution of 5-[6-chloro-3-[1-[2-(4,4-difluoro-1-piperidyl)-3,6-dimethyl-4-oxo-chromen-8-yl]ethylamino]-2-pyridyl]-3-fluoro-2-(4,4,5,5-tetramethyl-1,3,2-dioxaborolan-2-yl)benzaldehyde (1.00 g, 718.43 μmol, 1 eq) in THF (10 mL) was added NaBH₃CN (90.29 mg, 1.44 mmol, 2 eq) in portions at 0° C., the reaction mixture was warmed up to 25° C. and stirred at 25° C. for 0.5 h. The reaction mixture was quenched with H₂O (20 mL) and extracted with EtOAc (20 mL×3). The combined organic layers were washed with brine (20 mL×2), dried over Na₂SO₄, filtered and concentrated under reduced pressure to give a residue. The residue was purified by flash silica gel chromatography (Biotage®; 20 g SepaFlash® Silica Flash Column, Eluent of 0~100% Ethyl acetate: Methanol gradient @60 mL/min) and then the crude product was further purified by prep-HPLC (column: 2_Phenomenex Gemini C18 75×40 mm×3 um; mobile phase: [H₂O (10 mM NH₄HCO₃)-AC□]; gradient: 30%-70% B over 8.0 min) to give the title compound (120.00 mg, 200.73 μmol, 27.94% yield) as a white solid. ¹H NMR (DMSO-d₆, 400 MHz) δ 9.37 (s, 1H), 7.64 (s, 1H), 7.54 (s, 1H), 7.45 (s, 1H), 7.36 (d, J=9.2 Hz, 11H), 7.14 (d, J=8.4 Hz, 1H), 6.92 (d, J=8.8 Hz, 1H), 5.79 (d, J=6.8 Hz, 11H), 5.09-4.99 (m, 2H), 4.93 (t, J=6.4 Hz, 1H), 3.46 (t, J=5.2 Hz, 4H), 2.32 (s, 3H), 2.16-2.01 (m, 4H), 1.94 (s, 3H), 1.51 (d, J=6.4 Hz, 3H).

### Preparation of 8-[(1S)-1-[[6-chloro-2-(7-fluoro-1-hydroxy-3H-2,1-benzoxaborol-5-yl)-3-pyridyl]amino]ethyl]-2-(4,4-difluoro-1-piperidyl)-3,6-dimethyl-chromen-4-one and 8-[(1R)-1-[[6-chloro-2-(7-fluoro-1-hydroxy-3H-2,1-benzoxaborol-5-yl)-3-pyridyl]amino]ethyl]-2-(4,4-difluoro-1-piperidyl)-3,6-dimethyl-chromen-4-one

8-(1-((6-chloro-2-(7-fluoro-1-hydroxy-1,3-dihydrobenzo[c][1,2]oxaborol-5-yl)pyridin-3-yl)amino)ethyl)-2-(4,4-difluoropiperidin-1-yl)-3,6-dimethyl-4H-chromen-4-one (0.12 g, 200.73 μmol, 1 eq) was separated by SFC (column: DAICEL CHIRALCEL OD (250 mm×30 mm×10 um); mobile phase: [CO₂—IPA]; B %: 50%, isocratic elution mode) to give the title compound, Isomer 1 (32.00 mg, 53.27 μmol, 26.54% yield) as a white solid and the title compound, Isomer 2 (40.00 mg, 66.68 μmol, 33.22% yield) as a white solid. Isomer 1:1′H NMR (MeOD, 400 MHz) δ 7.76 (s, 1H), 7.44 (s, 1H), 7.35 (s, 1H), 7.19 (d, J=8.4 Hz, 1H), 7.09 (d, J=8.4 Hz, 1H), 6.95 (d, J=8.8 Hz, 1H), 5.02 (d, J=8.00 Hz, 2H), 5.00-4.98 (m, 1H), 3.55 (t, J=5.6 Hz, 4H), 2.37 (s, 3H), 2.19-2.12 (m, 4H), 2.05 (s, 3H), 1.56 (d, J=6.4 Hz, 3H). MS (ESI): mass calcd. For C₃₀H₂₈BClF₃N₄O₅ 597.18, m/z found 598.2 [M+H]⁺. HPLC: 99.52% (220 nm), 99.62% (254 nm). Chiral purity: 100% ee. Isomer 2: ¹H NMR (MeOD, 400 MHz) δ 7.76 (s, 1H), 7.44 (s, 1H), 7.34 (s, 1H), 7.18 (d, J=8.8 Hz, 1H), 7.10 (d, J=8.8 Hz, 1H), 6.94 (d, J=8.4 Hz, 1H), 5.01 (d, J=7.6 Hz, 2H), 4.98-4.96 (m, 1H), 3.55 (t, J=5.6 Hz, 4H), 2.37 (s, 3H), 2.20-2.12 (m, 4H), 2.05 (s, 3H), 1.56 (d, J=6.4 Hz, 3H). MS (ESI): mass calcd. For C₃₀H₂gBClF₃N₄O₅ 597.18, m/z found 598.1 [M+H]⁺. HPLC: 99.65% (220 nm), 99.59% (254 nm). Chiral purity: 98.00% ee

### Example 106

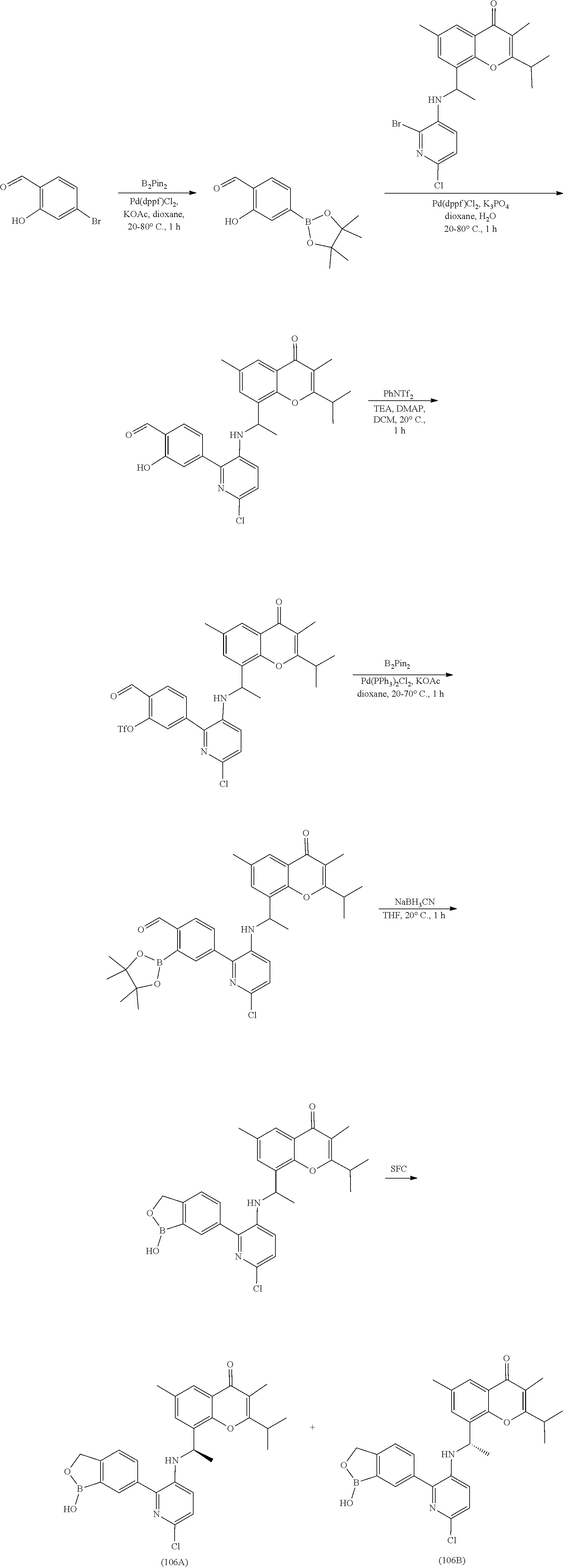

### Preparation of 2-hydroxy-4-(4,4,5,5-tetramethyl-1,3,2-dioxaborolan-2-yl)benzaldehyde

A mixture of 4-bromo-2-hydroxybenzaldehyde (5.00 g, 24.8 mmol, 1 eq), B₂Pin₂ (7.58 g, 29.8 mmol, 1.2 eq), KOAc (4.88 g, 49.7 mmol, 2 eq) and Pd(dppf)Cl₂ (546 mg, 746 μmol, 0.03 eq) in dioxane (80 mL) was degassed and purged with N₂ for 3 times at 20° C., and then the mixture was heated to 80° C. and stirred at 80° C. for 1 h under N₂ atmosphere. The reaction mixture was filtered and concentrated under reduced pressure to give a residue. The residue was purified by flash silica gel chromatography (Biotage®; 80 g SepaFlash® Silica Flash Column, Eluent of 0~10% Ethyl acetate/Petroleum ether gradient @80 mL/min) to give 2-hydroxy-4-(4,4,5,5-tetramethyl-1,3,2-dioxaborolan-2-yl)benzaldehyde (5.00 g, 20.15 mmol, 81.03% yield) as a white solid. ¹H NMR (CDCl₃, 400 MHz) δ 10.83 (s, 1H), 9.93 (s, 1H), 7.55 (d, J=8.0 Hz, 1H), 7.43-7.41 (m, 2H), 1.36 (s, 12H).

### Preparation of 4-(6-chloro-3-((1-(2-isopropyl-3,6-dimethyl-4-oxo-4H-chromen-8-yl) ethyl)amino)pyridin-2-yl)-2-hydroxybenzaldehyde

The title compound was prepared as a yellow solid (2.20 g, 4.48 mmol, 67.18% yield) according to the procedures described herein and the synthetic scheme, using 8-(1-((2-bromo-6-chloropyridin-3-yl)amino)ethyl)-2-isopropyl-3,6-dimethyl-4H-chromen-4-one (3.00 g, 6.67 mmol, 1 eq) and 2-hydroxy-4-(4,4,5,5-tetramethyl-1,3,2-dioxaborolan-2-yl) benzaldehyde (1.82 g, 7.34 mmol, 1.1 eq). ¹H NMR (CDCl₃, 400 MHz) δ 11.17 (s, 1H), 9.98 (s, 1H), 7.91 (s, 1H), 7.74 (d, J=8.0 Hz, 1H), 7.39-7.34 (m, 3H), 7.01 (d, J=8.8 Hz, 1H), 6.64 (d, J=8.8 Hz, 1H), 5.02-4.95 (m, 1H), 4.59 (d, J=5.2 Hz, 1H), 3.35-3.25 (m, 1H), 2.39 (s, 3H), 2.11 (s, 3H), 1.58 (d, J=6.4 Hz, 3H), 1.34 (d, J=6.4 Hz, 3H), 1.28 (d, J=6.8 Hz, 3H).

### Preparation of 5-(6-chloro-3-((1-(2-isopropyl-3,6-dimethyl-4-oxo-4H-chromen-8-yl) ethyl)amino)pyridin-2-yl)-2-formylphenyl trifluoromethanesulfonate

The title compound was prepared as a yellow solid (2.20 g, 3.53 mmol, 86.68% yield) according to the procedures described herein and the synthetic scheme, using 4-(6-chloro-3-((1-(2-isopropyl-3,6-dimethyl-4-oxo-4H-chromen-8-yl)ethyl)amino)pyridin-2-yl)-2-hydroxybenzaldehyde (2.00 g, 4.07 mmol, 1 eq) and PhNTf₂ (1.89 g, 5.30 mmol, 1.3 eq). ¹H NMR (CDCl₃, 400 MHz) δ 10.35 (s, 1H), 8.17 (d, J=8.0 Hz, 1H), 8.00 (d, J=8.0 Hz, 1H), 7.92 (s, 1H), 7.35 (d, J=2.0 Hz, 1H), 7.05 (d, J=8.8 Hz, 1H), 6.71 (d, J=8.8 Hz, 1H), 5.04-5.01 (m, 1H), 4.59 (s, 1H), 3.36-3.25 (m, 1H), 2.39 (s, 3H), 2.11 (s, 3H), 1.60 (d, J=6.8 Hz, 3H), 1.34 (d, J=6.8 Hz, 3H), 1.27-1.25 (m, 3H).

### Preparation of 4-(6-chloro-3-((1-(2-isopropyl-3,6-dimethyl-4-oxo-4H-chromen-8-yl) ethyl)amino)pyridin-2-yl)-2-(4,4,5,5-tetramethyl-1,3,2-dioxaborolan-2-yl)benzaldehyde

The title compound was prepared as yellow oil (500 mg, 832.03 μmol, 64.80% yield) according to the procedures described herein and the synthetic scheme, using 5-(6-chloro-3-((1-(2-isopropyl-3,6-dimethyl-4-oxo-4H-chromen-8-yl)ethyl)amino)pyridin-2-yl)-2-formylphenyl trifluoromethanesulfonate (800 mg, 1.28 mmol, 1 eq) and B₂Pin₂ (815 mg, 3.21 mmol, 2.5 eq). ¹H NMR (CDCl₃, 400 MHz) δ 10.66 (s, 1H), 8.27 (s, 1H), 8.14 (d, J=8.0 Hz, 1H), 7.94-7.90 (m, 2H), 7.32 (s, 1H), 7.00 (d, J=8.8 Hz, 1H), 6.62 (d, J=8.4 Hz, 1H), 5.03-4.98 (m, 1H), 4.53-4.45 (m, 1H), 3.33-3.26 (m, 1H), 2.39 (s, 3H), 2.11 (s, 3H), 1.53 (d, J=6.8 Hz, 3H), 1.41 (s, 12H), 1.35 (d, J=6.8 Hz, 3H).

### Preparation of 8-(1-((6-chloro-2-(1-hydroxy-1,3-dihydrobenzo[c][1,2]oxaborol-6-yl) pyridin-3-yl)amino)ethyl)-2-isopropyl-3,6-dimethyl-4H-chromen-4-one

A mixture of 4-(6-chloro-3-((1-(2-isopropyl-3,6-dimethyl-4-oxo-4H-chromen-8-yl)ethyl)amino)pyridin-2-yl)-2-(4,4,5,5-tetramethyl-1,3,2-dioxaborolan-2-yl)benzaldehyde (500 mg, 832 μmol, 1 eq) and NaBH₃CN (104 mg, 1.66 mmol, 2 eq) in THF (10 mL) was degassed and purged with N₂ for 3 times at 20° C., and then the mixture was stirred at 20° C. for 1 h under N₂ atmosphere. The reaction mixture was quenched by adding water (8 mL) at 0° C. and then filtered. The filter cake was further triturated with MeCN (10 mL) at 20° C. for 30 min to give the title compound (300 mg, 596.66 μmol, 71.71% yield) as a white solid. ¹H NMR (DMSO-d₆, 400 MHz) δ 9.29 (s, 1H), 8.07 (s, 1H), 7.85-7.82 (m, 1H), 7.68 (s, 1H), 7.56-7.53 (m, 2H), 7.13 (d, J=4.4 Hz, 1H), 6.86 (d, J=8.8 Hz, 1H), 5.52 (d, J=6.8 Hz, 1H), 5.06 (s, 2H), 5.01-4.96 (m, 1H), 3.29-3.24 (m, 1H), 2.34 (s, 3H), 1.98 (s, 3H), 1.51 (d, J=6.8 Hz, 3H), 1.24 (d, J=6.8 Hz, 3H), 1.14 (d, J=6.8 Hz, 3H).

### Preparation of (S)-8-(1-((6-chloro-2-(1-hydroxy-1,3-dihydrobenzo[c][1,2]oxaborol-6-yl) pyridin-3-yl)amino)ethyl)-2-isopropyl-3,6-dimethyl-4H-chromen-4-one and (R)-8-(1-((6-chloro-2-(1-hydroxy-1,3-dihydrobenzo[c][1,2]oxaborol-6-yl)pyridin-3-yl)amino)ethyl)-2-isopropyl-3,6-dimethyl-4H-chromen-4-one

8-(1-((6-chloro-2-(1-hydroxy-1,3-dihydrobenzo[c][1,2]oxaborol-6-yl)pyridin-3-yl)amino)ethyl)-2-isopropyl-3,6-dimethyl-4H-chromen-4-one (300 mg, 596 μmol, 1 eq) was separated by SFC (column: DAICEL CHIRALCEL OD (250 mm×30 mm, 10 um); mobile phase: [CO2-MeOH (0.1% □H3H2O)]; B %: 30%, isocratic elution mode) to give the title compound, Isomer 1 (122.7 mg, 241.69 μmol, 40.51% yield) as a white solid and the title compound, Isomer 2 (121.6 mg, 240.30 μmol, 40.27% yield) as a white solid. Isomer 1: ¹H NMR (DMSO-d₆, 400 MHz) δ 9.30 (s, 1H), 8.07 (s, 1H), 7.85-7.82 (m, 1H), 7.68 (s, 1H), 7.56-7.53 (m, 2H), 7.13 (d, J=8.4 Hz, 1H), 6.86 (d, J=8.8 Hz, 1H), 5.53 (d, J=7.2 Hz, 1H), 5.06 (s, 2H), 5.01-4.96 (m, 1H), 3.29-3.24 (m, 1H), 2.33 (s, 3H), 1.98 (s, 3H), 1.51 (d, J=6.4 Hz, 3H), 1.24 (d, J=6.8 Hz, 3H), 1.14 (d, J=6.4 Hz, 3H). MS (ESI): mass calcd. For C₂₈H₂₈BClN₂O₄ 502.18, m/z found 503.1 [M+H]⁺. HPLC: 99.04% (220 nm), 99.08% (254 nm). Chiral purity: 98.92% ee. Isomer 2: ¹H NMR (DMSO-d₆, 400 MHz) δ 9.30 (s, 1H), 8.07 (s, 1H), 7.85-7.82 (m, 1H), 7.68 (s, 1H), 7.56-7.53 (m, 2H), 7.13 (d, J=8.4 Hz, 1H), 6.86 (d, J=8.8 Hz, 1H), 5.52 (d, J=6.8 Hz, 1H), 5.06 (s, 2H), 5.01-4.96 (m, 1H), 3.29-3.24 (m, 1H), 2.33 (s, 3H), 1.98 (s, 3H), 1.51 (d, J=6.4 Hz, 3H), 1.24 (d, J=6.8 Hz, 3H), 1.14 (d, J=6.4 Hz, 3H). MS (ESI): mass calcd. For C₂₈H₂₈BClN₂O₄ 502.18, m/z found 503.1 [M+H]⁺. HPLC: 99.36% (220 nm), 99.60% (254 nm). Chiral purity: 96.08% ee

### Example 107

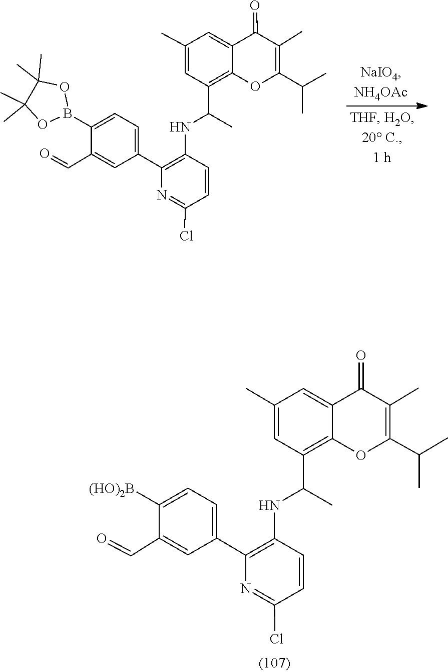 [SMILES: Cc1cc(C(C)Nc2ccc(Cl)nc2-c2ccc(B3OC(C)(C)C(C)(C)O3)c(C=O)c2)c2oc(C(C)C)c(C)c(=O)c2c1 ; Cc1cc(C(C)Nc2ccc(Cl)nc2-c2ccc(B(O)O)c(C=O)c2)c2oc(C(C)C)c(C)c(=O)c2c1]

### Preparation of [4-[6-chloro-3-[1-(2-isopropyl-3,6-dimethyl-4-oxo-chromen-8-yl) ethylamino]-2-pyridyl]-2-formyl-phenyl]boronic acid

To a solution of 5-[6-chloro-3-[1-(2-isopropyl-3,6-dimethyl-4-oxo-chromen-8-yl)ethylamino]-2-pyridyl]-2-(4,4,5,5-tetramethyl-1,3,2-dioxaborolan-2-yl)benzaldehyde (1.00 g, 1.66 mmol, 1 eq) in H₂O (10 mL)/THF (10 mL) was added NH₄OAc (385 mg, 4.99 mmol, 3 eq) and NaIO4 (1.07 g, 4.99 mmol, 276.63 μL, 3 eq) in turns at 20° C., the reaction mixture was stirred at 20° C. for 1 h. The reaction mixture was filtered and the filtrate was extracted with EtOAc (20 mL×3). The combined organic phase was washed with brine (10 mL), dried with anhydrous Na₂SO₄, filtered and concentrated in vacuum. 500 mg of the crude product was purified by prep-HPLC (column: Phenomenex luna C18 100×40 mm×5 um; mobile phase: [H₂O (0.2% FA)-ACN]; gradient: 50%-95% B over 8.0 min) to give the title compound (300 mg, 578.26 μmol, 60.00% yield) as a yellow solid. ¹H NMR (DMSO-d₆, 400 MHz) δ 10.25 (s, 1H), 8.36 (s, 2H), 8.21 (s, 1H), 8.02-8.00 (m, 1H), 7.77 (d, J=7.6 Hz, 1H), 7.69 (s, 1H), 7.57 (s, 1H), 7.16 (d, J=8.4 Hz, 1H), 6.84 (d, J=8.8 Hz, 1H), 5.70-5.68 (m, 1H), 5.06-4.99 (m, 1H), 3.28-3.24 (m, 1H), 2.34 (s, 3H), 1.99 (s, 3H), 1.52-1.51 (m, 3H), 1.26 (d, J=7.6 Hz, 1H), 1.20 (d, J=7.2 Hz, 1H). MS (ESI): mass calcd. For C28H28BClN2O5 518.18, m/z found 519.2 [M+H]⁺. HPLC: 99.41% (220 nm), 99.54% (254 nm)

### Example 108

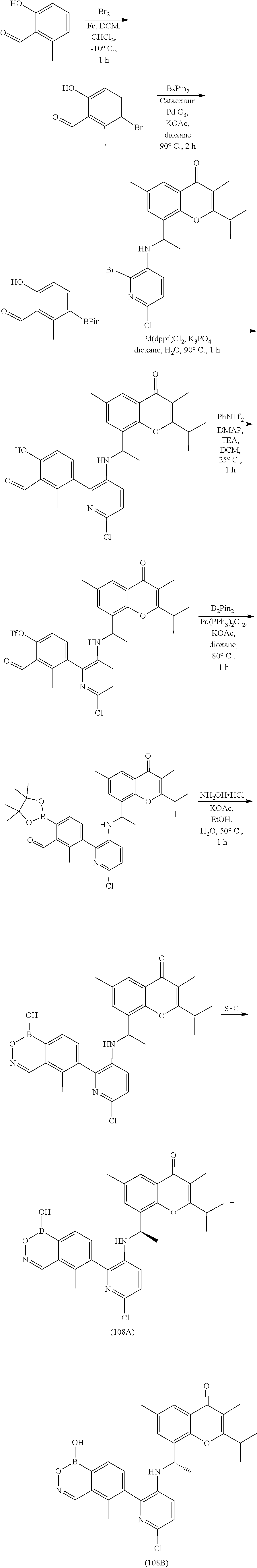

### Preparation of 3-bromo-6-hydroxy-2-methyl-benzaldehyde

To a mixture of 2-hydroxy-6-methyl-benzaldehyde (4.00 g, 29.3 mmol, 1 eq) and Fe (82.0 mg, 1.47 mmol, 0.05 eq) in CHCl₃ (40 mL) was added a solution of Br₂ (4.93 g, 30.8 mmol, 1.59 mL, 1.05 eq) in DCM (5 mL) dropwise at −10° C., the reaction mixture was stirred at −10° C. for 1 h. To the reaction mixture was added H₂O (100 mL) at −10° C., and then extracted with DCM (100 mL×3). The combined organic layers were washed with sodium thiosulfate solution (100 mL), brine (100 mL), dried over Na₂SO₄, filtered and concentrated under reduced pressure to give the title compound (6.30 g, 29.3 mmol, 99.7% yield) as a yellow solid. ¹H NMR (CDCl₃, 400 MHz) δ 12.07 (s, 1H), 10.38 (s, 1H), 7.64 (d, J=8.8 Hz, 1H), 6.76 (d, J=8.8 Hz, 1H), 2.70 (s, 3H)

### Preparation of 6-hydroxy-2-methyl-3-(4,4,5,5-tetramethyl-1,3,2-dioxaborolan-2-yl) benzaldehyde

To a mixture of 3-bromo-6-hydroxy-2-methyl-benzaldehyde (5.00 g, 23.2 mmol, 1 eq) and 4,4,5,5-tetramethyl-2-(4,4,5,5-tetramethyl-1,3,2-dioxaborolan-2-yl)-1,3,2-dioxaborolane (8.86 g, 34.8 mmol, 1.5 eq) in dioxane (50 mL) were added [2-(2-aminophenyl)phenyl]palladium(1+);bis(1-adamantyl)-butyl-phosphane;methanesulfonate (846 mg, 1.16 mmol, 0.05 eq) and KOAc (6.85 g, 69.7 mmol, 3 eq) in turns at 20° C., the reaction mixture was degassed and purged with N₂ for 3 times, and then the reaction mixture was directly stirred at 90° C. (the oil-bath was pre-heated to 90° C.) for 2 h under N₂ atmosphere. The reaction mixture was cooled to 25° C., filtered and the filter cake was washed with EtOAc (50 mL). The filtrate was concentrated under reduced pressure and the residue was purified by flash silica gel chromatography (BIOTAGE®; 80 g SepaFlash® Silica Flash Column, Eluent of 0~10% Ethyl acetate/Petroleum ether gradient @100 mL/min) to give the title compound (4.50 g, 17.1 mmol, 73.8% yield) as a black solid. ¹H NMR (CDCl₃, 400 MHz) δ 12.34 (s, 1H), 10.44 (s, 1H), 7.91 (d, J=8.8 Hz, 1H), 6.80 (d, J=8.8 Hz, 1H), 2.86 (s, 3H), 1.35 (s, 12H).

### Preparation of 3-[6-chloro-3-[1-(2-isopropyl-3,6-dimethyl-4-oxo-chromen-8-yl) ethyl amino]-2-pyridyl]-6-hydroxy-2-methyl-benzaldehyde

The title compound was prepared as a yellow solid (2.00 g, 3.96 mmol, 89.06% yield) according to the similar procedure in Example 79 (utilizing the modification depicted in the Example 108 synthetic scheme), using 8-[1-[(2-bromo-6-chloro-3-pyridyl)amino]ethyl]-2-isopropyl-3,6-dimethyl-chromen-4-one (2.00 g, 4.45 mmol, 1 eq) and 6-hydroxy-2-methyl-3-(4,4,5,5-tetramethyl-1,3,2-dioxaborolan-2-yl)benzaldehyde (1.75 g, 6.67 mmol, 1.5 eq). ¹H NMR (CDCl₃, 400 MHz) δ 12.23 (s, 1H), 10.47 (s, 1H), 7.90 (s, 1H), 7.48 (d, J=8.4 Hz, 1H), 7.34-7.19 (m, 1H), 7.09-6.99 (m, 2H), 6.63 (t, J=9.6 Hz, 1H), 5.10-4.77 (m, 2H), 3.37-3.26 (m, 1H), 2.50 (s, 3H), 2.38 (d, J=2.4 Hz, 3H), 2.11 (s, 3H), 1.51 (t, J=6.0 Hz, 3H), 1.37 (d, J=6.8 Hz, 3H), 1.31 (d, J=6.8 Hz, 3H).

### Preparation of [4-[6-chloro-3-[1-(2-isopropyl-3,6-dimethyl-4-oxo-chromen-8-yl) ethylamino]-2-pyridyl]-2-formyl-3-methyl-phenyl]trifluoromethanesulfonate

The title compound was prepared as a yellow solid (1.40 g, 2.20 mmol, 55.49% yield) according to the similar procedure in Example 79 (utilizing the modification depicted in the Example 108 synthetic scheme), using 3-[6-chloro-3-[1-(2-isopropyl-3,6-dimethyl-4-oxo-chromen-8-yl)ethylamino]-2-pyridyl]-6-hydroxy-2-methyl-benzaldehyde (2.00 g, 3.96 mmol, 1 eq) and PhNTf₂ (1.84 g, 5.15 mmol, 1.3 eq). ¹H NMR (DMSO-d₆, 400 MHz) δ 10.52-10.51 (m, 1H), 7.82-7.65 (m, 2H), 7.55 (d, J=8.4 Hz, 1H), 7.49-7.39 (m, 1H), 7.28-7.20 (m, 1H), 6.96-6.78 (m, 1H), 5.65-5.44 (m, 1H), 5.13-4.98 (m, 1H), 3.40-3.38 (m, 1H), 2.44 (s, 3H), 2.36-2.33 (m, 3H), 2.03-2.00 (m, 3H), 1.48 (d, J=3.6 Hz, 3H), 1.33 (d, J=6.0 Hz, 3H), 1.27 (d, J=6.8 Hz, 3H)

### Preparation of 3-[6-chloro-3-[1-(2-isopropyl-3,6-dimethyl-4-oxo-chromen-8-yl)ethyl amino]-2-pyridyl]-2-methyl-6-(4,4,5,5-tetramethyl-1,3,2-dioxaborolan-2-yl)benzaldehyde

The title compound was prepared as yellow oil (0.7 g, 1.14 mmol, 48.34% yield) according to the similar procedure in Example 79, using [4-[6-chloro-3-[1-(2-isopropyl-3,6-dimethyl-4-oxo-chromen-8-yl)ethylamino]-2-pyridyl]-2-formyl-3-methyl-phenyl]trifluoromethanesulfonate (1.50 g, 2.35 mmol, 1 eq) and B₂Pin₂ (2.39 g, 9.42 mmol, 4 eq). ¹H NMR (DMSO-d₆, 400 MHz) δ 10.49 (s, 1H), 7.94 (s, 2H), 7.69 (s, 1H), 7.50 (s, 1H), 7.19 (d, J=8.4 Hz, 1H), 6.93-6.74 (m, 1H), 5.20-5.14 (m, 1H), 5.09-4.95 (m, 1H), 3.30-3.29 (m, 1H), 2.41 (s, 3H), 2.32 (s, 3H), 1.99 (s, 3H), 1.45 (s, 3H), 1.37 (s, 12H), 1.31 (d, J=7.2 Hz, 3H), 1.24 (d, J=6.8 Hz, 3H)

### Preparation of 8-[1-[[6-chloro-2-(1-hydroxy-5-methyl-2,3,1-benzoxazaborinin-6-yl)-3-pyridyl]amino]ethyl]-2-isopropyl-3,6-dimethyl-chromen-4-one

The title compound was prepared as a yellow solid (340 mg, 641.73 mol) according to the similar procedure in Example 79 (utilizing the modification depicted in the Example 108 synthetic scheme), using 3-[6-chloro-3-[1-(2-isopropyl-3,6-dimethyl-4-oxo-chromen-8-yl)ethylamino]-2-pyridyl]-2-methyl-6-(4,4,5,5-tetramethyl-1,3,2-dioxaborolan-2-yl)benzaldehyde (500 mg, 813 μmol, 1 eq) and NH₂OH·HCl (56.5 mg, 813 μmol, 1 eq). ¹H NMR (DMSO-d₆, 400 MHz) δ 9.44-9.43 (m, 1H), 8.94-8.82 (m, 1H), 8.03-8.02 (m, 1H), 7.77-7.65 (m, 1H), 7.63-7.53 (m, 1H), 7.50-7.33 (m, 1H), 7.26-7.17 (m, 1H), 7.00-6.80 (m, 1H), 5.34-5.16 (m, 1H), 5.10-4.94 (m, 1H), 3.32-3.27 (m, 1H), 2.35-2.31 (m, 6H), 1.99 (s, 3H), 1.44 (s, 3H), 1.29 (d, J=3.6 Hz, 3H), 1.21 (t, J=5.2 Hz, 3H)

### Preparation of (S)-8-(1-((6-chloro-2-(1-hydroxy-5-methyl-JH-benzo[d][1,2,6]oxazaborinin-6-yl)pyridin-3-yl)amino)ethyl)-2-isopropyl-3,6-dimethyl-4H-chromen-4-one and (R)-8-(1-((6-chloro-2-(1-hydroxy-5-methyl-JH-benzo[d][1,2,6]oxazaborinin-6-yl)pyridin-3-yl)amino)ethyl)-2-isopropyl-3,6-dimethyl-4H-chromen-4-one

8-[1-[[6-chloro-2-(1-hydroxy-5-methyl-2,3,1-benzoxazaborinin-6-yl)-3-pyridyl]amino]ethyl]-2-isopropyl-3,6-dimethyl-chromen-4-one (350 mg, 0.66 mmol) was separated by SFC (column: DAICEL CHIRALPAK IG (250 mm×50 mm, 10 um); mobile phase: [CO2-IPA]; B %: 55%, isocratic elution mode) to give the title compound, Isomer 1 (105.6 mg, 211 gmol, 30.0% yield) as a yellow solid and the title compound, isomer 2 (102.8 mg, 250 μmol, 29.0% yield) as a yellow solid. Isomer 1: ¹H NMR (DMSO-d₆, 400 MHz) δ 9.41-9.39 (m, 1H), 8.91-8.85 (m, 1H), 8.02-8.00 (m, 1H), 7.68-7.65 (m, 1H), 7.58-7.56 (m, 1H), 7.53-7.35 (m, 1H), 7.20 (d, J=7.2 Hz, 1H), 7.02-6.80 (m, 1H), 5.33-5.19 (m, 1H), 5.02 (s, 1H), 3.31-3.23 (m, 1H), 2.36-2.25 (m, 6H), 1.99 (s, 3H), 1.47-1.40 (m, 3H), 1.29 (d, J=4.4 Hz, 3H), 1.21-1.20 (m, 3H). MS (ESI): mass calcd. For C₂₉H₂₉BClN₃O₄ 529.19, m/z found 530.2 [M+H]⁺. HPLC: 96.68% (220 nm), 96.94% (254 nm). Chiral purity: 100% ee. Isomer 2: ¹H NMR (DMSO-d₆, 400 MHz) δ 9.41-9.39 (m, 1H), 8.91-8.85 (m, 1H), 8.02-8.00 (m, 1H), 7.68-7.65 (m, 1H), 7.58-7.56 (m, 1H), 7.51-7.33 (m, 1H), 7.20 (t, J=6.4 Hz, 1H), 6.98-6.78 (m, 1H), 5.41-5.17 (m, 1H), 5.01 (s, 1H), 3.30-3.21 (m, 1H), 2.36-2.25 (m, 6H), 1.99 (s, 3H), 1.47-1.40 (m, 3H), 1.28 (s, 3H), 1.21-1.20 (m, 3H). MS (ESI): mass calcd. For C₂₉H₂₉BClN₃O₄ 529.19, m/z found 530.2 [M+H]⁺. HPLC: 99.34% (220 nm), 99.64% (254 nm). Chiral purity: 99.34% ee.

### Example 109

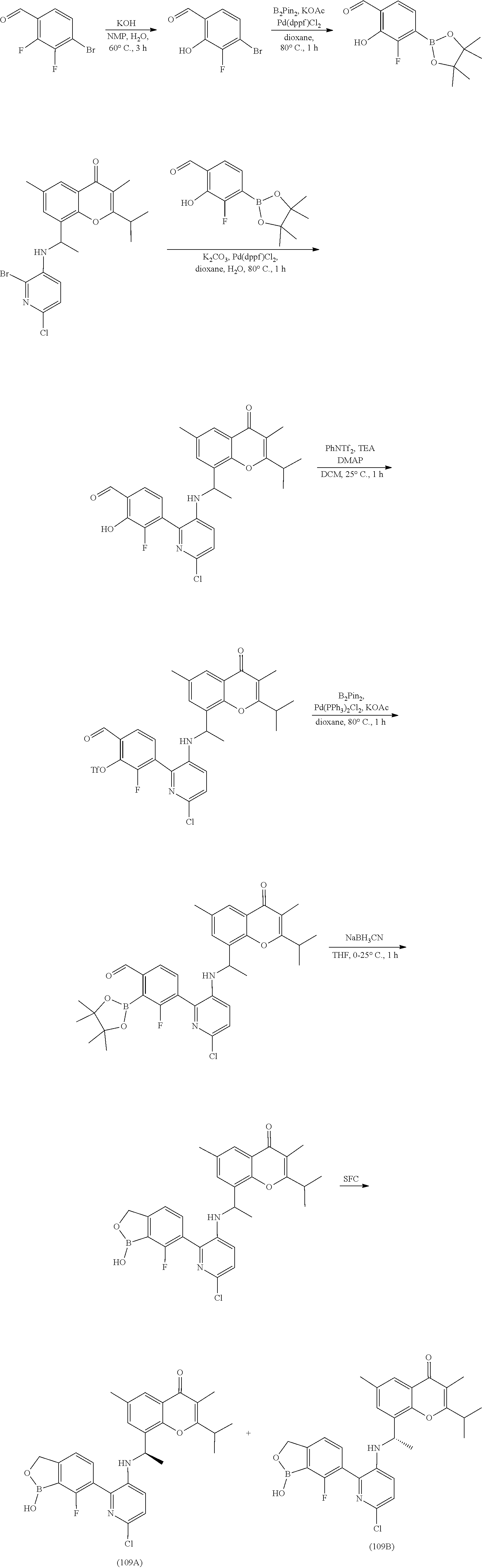

### Preparation of 4-bromo-3-fluoro-2-hydroxy-benzaldehyde

A mixture of 4-bromo-2,3-difluoro-benzaldehyde (10.00 g, 45.25 mmol, 1 eq) and KOH (3.30 g, 58.82 mmol, 1.3 eq) in NMP (120 mL)/H₂O (20 mL) was stirred at 60° C. for 3 h. The reaction mixture was cooled to 25° C., diluted with H₂O (500 mL), adjusted pH=4 with 1 N HCl and extracted with DCM (100 mL×2). The combined organics were washed with brine (100 mL×2), dried over Na₂SO₄, filtered and concentrated in vacuo. The residue was purified by a silica gel column chromatography (PE:EtOAc=10:1) to give the title compound (5.00 g, 22.83 mmol, 50.45% yield) as light yellow oil. ¹H NMR (DMSO-d₆, 400 MHz) δ 11.35 (s, 1H), 10.24 (s, 1H), 7.41 (dd, J=1.6, 8.4 Hz, 1H) 7.25 (dd, J=6.0, 8.4 Hz, 1H).

### Preparation of 3-fluoro-2-hydroxy-4-(4,4,5,5-tetramethyl-1,3,2-dioxaborolan-2-yl) benzaldehyde

A mixture of 4-bromo-3-fluoro-2-hydroxy-benzaldehyde (2.10 g, 9.59 mmol, 1 eq), B₂Pin₂ (6.09 g, 23.97 mmol, 2.5 eq), KOAc (1.22 g, 12.47 mmol, 1.3 eq) and [2-(2-aminophenyl)phenyl]palladium(1+);bis(1-adamantyl)-butyl-phosphane;methanesulfonate (698 mg, 958.87 μmol, 0.1 eq) in dioxane (50 mL) was degassed and purged with N₂ for 3 times at 25° C., and then the reaction mixture was heated to 80° C. and stirred at 80° C. for 1 h under N₂ atmosphere. The reaction mixture was cooled to 25° C., filtered and the filtrate was concentrated under reduced pressure. The residue was purified by flash silica gel chromatography (ISCO®; 330 g SepaFlash® Silica Flash Column, Eluent of 0~5% Ethyl acetate/Petroleum ether gradient @200 mL/min) to give the title compound (2.20 g, 8.27 mmol, 86.23% yield) as a green solid. ¹H NMR (DMSO-d₆, 400 MHz) δ 10.81 (s, 1H), 10.31 (s, 1H), 7.45 (d, J=8.0 Hz, 1H), 7.13 (dd, J=4.8, 7.6 Hz, 1H), 1.30 (s, 12H).

### Preparation of 4-[6-chloro-3-[1-(2-isopropyl-3,6-dimethyl-4-oxo-chromen-8-yl) ethylamino]-2-pyridyl]-3-fluoro-2-hydroxy-benzaldehyde

The title compound was prepared as a yellow solid (5.50 g, 10.81 mmol, 93.47% yield) according to the procedures described herein and the synthetic scheme, using 8-[1-[(2-bromo-6-chloro-3-pyridyl)amino]ethyl]-2-isopropyl-3,6-dimethyl-chromen-4-one (5.2 g, 11.56 mmol, 1 eq) and 3-fluoro-2-hydroxy-4-(4,4,5,5-tetramethyl-1,3,2-dioxaborolan-2-yl)benzaldehyde (6.15 g, 23.12 mmol, 2 eq). ¹H NMR (DMSO-d₆, 400 MHz) δ 11.06 (s, 1H) 10.36 (s, 1H), 7.69 (d, J=1.2 Hz, 1H), 7.59 (dd, J=0.8, 8.0 Hz, 1H), 7.49 (d, J=2.0 Hz, 1H), 7.19 (d, J=8.8 Hz, 1H), 7.04 (dd, J=6.0, 8.0 Hz, 1H), 6.81 (d, J=8.8 Hz, 1H), 5.75 (d, J=7.2 Hz, 1H), 5.04 (q, J=6.8 Hz, 1H), 3.30-3.29 (m, 1H), 2.33 (s, 3H), 1.50 (d, J=6.8 Hz, 3H), 1.31 (d, J=6.8 Hz, 3H), 1.25 (d, J=6.8 Hz, 3H), 1.16 (d, J=6.4 Hz, 3H).

### Preparation of [3-[6-chloro-3-[1-(2-isopropyl-3,6-dimethyl-4-oxo-chromen-8-yl) ethylamino]-2-pyridyl]-2-fluoro-6-formyl-phenyl]trifluoromethanesulfonate

The title compound was prepared as a yellow solid (3.80 g, 5.93 mmol, 60.34% yield) according to the procedures described herein and the synthetic scheme, using 4-[6-chloro-3-[1-(2-isopropyl-3,6-dimethyl-4-oxo-chromen-8-yl)ethylamino]-2-pyridyl]-3-fluoro-2-hydroxy-benzaldehyde (5.00 g, 9.82 mmol, 1 eq) and 1,1,1-trifluoro-N-phenyl-N-(trifluoromethylsulfonyl)methanesulfonamide (5.26 g, 14.74 mmol, 1.5 eq). ¹H NMR (DMSO-d₆, 400 MHz) δ 10.17 (s, 1H), 8.09 (d, J=8.0 Hz, 1H), 7.96 (t, J=6.4 Hz, 1H), 7.69 (s, 1H), 7.50 (d, J=1.6 Hz, 1H), 7.26 (d, J=8. Hz, 1H), 6.86 (d, J=8.8 Hz, 1H), 6.12 (d, J=6.4 Hz, 1H), 5.09-5.05 (m, 1H), 3.32-3.28 (m, 1H), 2.32 (s, 3H), 2.00 (s, 3H), 1.48 (d, J=6.8 Hz, 3H), 1.31 (d, J=6.8 Hz, 3H), 1.27 (d, J=6.8 Hz, 3H).

### Preparation of 4-[6-chloro-3-[1-(2-isopropyl-3,6-dimethyl-4-oxo-chromen-8-yl) ethylamino]-2-pyridyl]-3-fluoro-2-(4,4,5,5-tetramethyl-1,3,2-dioxaborolan-2-yl)benzaldehyde

The title compound was prepared as a green solid (2.70 g, 4.36 mmol, 90.21% yield) according to the procedures described herein and the synthetic scheme, using [3-[6-chloro-3-[1-(2-isopropyl-3,6-dimethyl-4-oxo-chromen-8-yl)ethylamino]-2-pyridyl]-2-fluoro-6-formyl-phenyl]trifluoromethanesulfonate (3.1 g, 4.84 mmol, 1 eq) and B₂Pin₂ (3.68 g, 14.51 mmol, 3 eq). ¹H NMR (DMSO-d₆+D₂O, 400 MHz) δ 10.03 (d, J=2.4 Hz, 1H), 7.91 (d, J=7.6 Hz, 1H), 7.68-7.65 (m, 2H), 7.45 (d, J=2.0 Hz, 1H), 7.17 (d, J=8.8 Hz, 1H), 7.08-7.01 (m, 1H), 6.80 (d, J=8.8 Hz, 1H), 5.07-5.01 (m, 1H), 3.35-3.24 (m, 1H), 2.30 (s, 3H), 1.98 (s, 3H), 1.46 (d, J=6.8 Hz, 3H), 1.28 (d, J=6.8 Hz, 3H), 1.23 (d, J=6.8 Hz, 3H), 1.23 (s, 12H).

### Preparation of 8-[1-[[6-chloro-2-(7-fluoro-1-hydroxy-3H-2,1-benzoxaborol-6-yl)-3-pyridyl]amino]ethyl]-2-isopropyl-3,6-dimethyl-chromen-4-one

To a solution of 4-[6-chloro-3-[1-(2-isopropyl-3,6-dimethyl-4-oxo-chromen-8-yl) ethylamino]-2-pyridyl]-3-fluoro-2-(4,4,5,5-tetramethyl-1,3,2-dioxaborolan-2-yl)benzaldehyde (2.50 g, 4.04 mmol, 1 eq) in THF (30 mL) was added NaBH₃CN (508 mg, 8.08 mmol, 2 eq) in portions at 0° C., the reaction mixture was stirred at 25° C. for 1 h. The reaction mixture was quenched by H₂O (30 mL) and extracted with EtOAc (30 mL×3). The combined organic layers were washed with brine (30 mL), dried over anhydrous Na₂SO₄, filtered and concentrated under reduced pressure to give a residue. The residue was purified by flash silica gel chromatography (ISCO®; 40 g SepaFlash® Silica Flash Column, Eluent of 0~35% Ethyl acetate/Petroleum ether gradient @100 mL/min) and then prep-HPLC (column: Waters Xbridge BEH C18 250×50 mm×10 um; mobile phase: [H2O (10 mM NH₄HCO₃)-ACN]; gradient: 30%-60% B over 10.0 min) to give the title compound (370 mg, 710.46 μmol, 17.59% yield) as a white solid. ¹H NMR (DMSO-d₆+D₂O, 400 MHz) δ 7.65 (s, 1H), 7.52-7.46 (m, 2H), 7.31 (d, J=7.6 Hz, 1H), 7.15 (d, J=8.8 Hz, 1H), 6.85-6.84 (m, 1H), 5.28 (s, 1H), 5.00 (s, 2H), 4.95 (q, J=6.8 Hz, 1H), 3.30-3.23 (m, 1H), 2.31 (s, 3H), 1.96 (s, 3H), 1.45 (d, J=6.83 Hz, 3H), 1.25 (d, J=6.8 Hz, 3H), 1.16 (d, J=6.4 Hz, 3H).

### Preparation of (S)-8-(1-((6-chloro-2-(7-fluoro-1-hydroxy-1,3-dihydrobenzo[c][1,2]oxaborol-6-yl)pyridin-3-yl)amino)ethyl)-2-isopropyl-3,6-dimethyl-4H-chromen-4-one and (R)-8-(1-((6-chloro-2-(7-fluoro-1-hydroxy-1,3-dihydrobenzo[c][1,2]oxaborol-6-yl)pyridin-3-yl)amino)ethyl)-2-isopropyl-3,6-dimethyl-4H-chromen-4-one

8-(1-((6-chloro-2-(7-fluoro-1-hydroxy-1,3-dihydrobenzo[c][1,2]oxaborol-6-yl)pyridin-3-yl)amino)ethyl)-2-isopropyl-3,6-dimethyl-4H-chromen-4-one (0.35 g, 672.06 μmol, 1 eq) was separated by SFC (column: ChiralPak IH, 250×30 mm×10 um; mobile phase: [CO2-IPA]; B %:35%, isocratic elution mode) to give the title compound, Isomer 1 (133.5 mg, 255.42 μmol, 38.01% yield) as a white solid and the title compound, Isomer 2 (136.1 mg, 258.80 μmol, 38.51% yield) as a white solid. Isomer 1: ¹H NMR (DMSO-d₆, 400 MHz) δ 9.33 (s, 1H), 7.68 (s, 1H), 7.57 (t, J=7.6 Hz, 1H), 7.49 (d, J=2.0 Hz, 1H), 7.35 (d, J=8.0 Hz, 1H), 7.17 (d, J=8.8 Hz, 1H), 6.83 (d, J=8.8 Hz, 1H), 5.42 (d, J=6.8 Hz, 1H), 5.07 (s, 2H), 5.04-4.96 (m, 1H), 3.31-3.27 (m, 1H), 2.33 (s, 3H), 2.00 (s, 3H), 1.48 (d, J=6.8 Hz, 3H), 1.30 (d, J=6.4 Hz, 3H), 1.22 (d, J=6.4 Hz, 3H). MS (ESI): mass calcd. For C₂₈H27BClFN₂O₄ 520.17, m/z found 521.2 [M+H]⁺. HPLC: 99.64% (220 nm), 99.81% (254 nm). Chiral purity: 100% ee. Isomer 2: ¹H NMR (DMSO-d₆, 400 MHz) δ 9.33 (s, 1H), 7.68 (s, 1H), 7.57 (t, J=7.6 Hz, 1H), 7.49 (d, J=2.0 Hz, 1H), 7.35 (d, J=8.0 Hz, 1H), 7.17 (d, J=8.8 Hz, 1H), 6.83 (d, J=8.8 Hz, 1H), 5.42 (d, J=6.8 Hz, 1H), 5.07 (s, 2H), 5.04-4.96 (m, 1H), 3.31-3.27 (m, 1H), 2.33 (s, 3H), 2.00 (s, 3H), 1.48 (d, J=6.8 Hz, 3H), 1.30 (d, J=6.4 Hz, 3H), 1.22 (d, J=6.4 Hz, 3H). MS (ESI): mass calcd. For C₂₈H₂₇BClFN₂O₄ 520.17, m/z found 521.2 [M+H]⁺. HPLC: 99.03% (220 nm), 99.39% (254 nm). Chiral purity: 97.44% ee

### Example 110

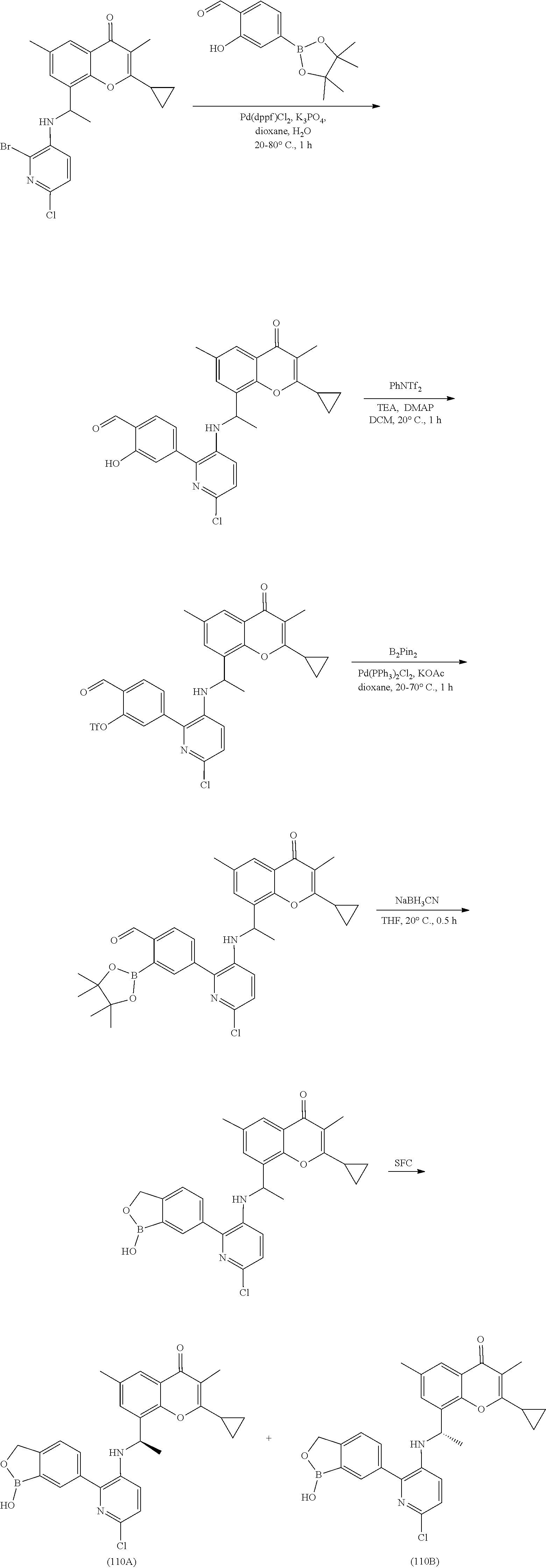

### Preparation of 4-(6-chloro-3-((1-(2-cyclopropyl-3,6-dimethyl-4-oxo-4H-chromen-8-yl) ethyl)amino)pyridin-2-yl)-2-hydroxybenzaldehyde

The title compound was prepared as a yellow solid (4.90 g, 10.02 mmol, 99.71% yield) according to the procedures described herein and the synthetic scheme, using 8-(1-((2-bromo-6-chloropyridin-3-yl)amino)ethyl)-2-cyclopropyl-3,6-dimethyl-4H-chromen-4-one (4.50 g, 10.05 mmol, 1 eq) and 2-hydroxy-4-(4,4,5,5-tetramethyl-1,3,2-dioxaborolan-2-yl) benzaldehyde (2.74 g, 11.06 mmol, 1.1 eq). ¹H NMR (CDCl₃, 400 MHz) δ 11.18 (s, 1H), 9.98 (s, 1H), 7.90 (d, J=1.2 Hz, 1H), 7.75 (d, J=8.4 Hz, 1H), 7.38-7.36 (m, 2H), 7.30 (d, J=2.0 Hz, 1H), 7.01 (d, J=8.8 Hz, 1H), 6.57 (d, J=8.8 Hz, 1H), 4.84-4.78 (m, 1H), 4.53 (d, J=4.8 Hz, 1H), 2.38 (s, 3H), 2.21 (s, 3H), 2.18-2.17 (m, 1H), 1.52 (d, J=6.4 Hz, 3H), 1.18-1.10 (m, 4H).

### Preparation of 5-(6-chloro-3-((1-(2-cyclopropyl-3,6-dimethyl-4-oxo-4H-chromen-8-yl) ethyl)amino)pyridin-2-yl)-2-formylphenyl trifluoromethanesulfonate

The title compound was prepared as a yellow solid (2.20 g, 3.54 mmol, 72.17% yield) according to the procedures described herein and the synthetic scheme, using 4-(6-chloro-3-((1-(2-cyclopropyl-3,6-dimethyl-4-oxo-4H-chromen-8-yl)ethyl)amino)pyridin-2-yl)-2-hydroxybenzaldehyde (2.40 g, 4.91 mmol, 1 eq) and PhNTf₂ (2.28 g, 6.38 mmol, 1.3 eq). ¹H NMR (CDCl₃, 400 MHz) δ 10.33 (s, 1H), 8.17 (d, J=8.0 Hz, 1H), 8.00-7.98 (m, 1H), 7.90-7.90 (m, 2H), 7.31 (s, 1H), 7.05 (d, J=8.4 Hz, 1H), 6.63 (d, J=8.8 Hz, 1H), 4.87-4.54 (m, 1H), 4.54-4.53 (m, 1H), 2.37 (s, 3H), 2.21 (s, 3H), 2.17-2.15 (m, 1H), 1.54 (d, J=6.8 Hz, 3H), 1.16-1.08 (m, 4H).

### Preparation of 4-(6-chloro-3-((1-(2-cyclopropyl-3,6-dimethyl-4-oxo-4H-chromen-8-yl) ethyl)amino)pyridin-2-yl)-2-(4,4,5,5-tetramethyl-1,3,2-dioxaborolan-2-yl)benzaldehyde

The title compound was prepared as yellow oil (550 mg, 918.32 gmol, 57.03% yield) according to the procedures described herein and the synthetic scheme, using 5-(6-chloro-3-((1-(2-cyclopropyl-3,6-dimethyl-4-oxo-4H-chromen-8-yl)ethyl)amino)pyridin-2-yl)-2-formylphenyl trifluoromethanesulfonate (1.00 g, 1.61 mmol, 1 eq) and B₂Pin₂ (1.02 g, 4.03 mmol, 2.5 eq). ¹H NMR (CDCl₃, 400 MHz) δ 10.66 (s, 1H), 8.26 (d, J=1.6 Hz, 1H), 8.14 (d, J=8.0 Hz, 1H), 7.93-7.89 (m, 2H), 7.29 (d, J=2.0 Hz, 1H), 7.00 (d, J=8.8 Hz, 1H), 6.56 (d, J=8.8 Hz, 1H), 4.85-4.80 (m, 1H), 4.46-4.40 (m, 1H), 2.37 (s, 3H), 2.21 (s, 3H), 2.19-2.18 (m, 1H), 1.47 (d, J=6.8 Hz, 3H), 1.41 (s, 12H), 1.18-1.09 (m, 4H).

### Preparation of 8-(1-((6-chloro-2-(1-hydroxy-1,3-dihydrobenzo[c][1,2]oxaborol-6-yl) pyridine-3-yl)amino)ethyl)-2-cyclopropyl-3,6-dimethyl-4H-chromen-4-one

A mixture of 4-(6-chloro-3-((1-(2-cyclopropyl-3,6-dimethyl-4-oxo-4H-chromen-8-yl)ethyl)amino)pyridin-2-yl)-2-(4,4,5,5-tetramethyl-1,3,2-dioxaborolan-2-yl)benzaldehyde (550 mg, 918 mol, 1 eq) and NaBH₃CN (115 mg, 1.84 mmol, 2 eq) in THF (10 mL) was degassed and purged with N₂ for 3 times at 20° C., and then the mixture was stirred at 20° C. for 0.5 h under N₂ atmosphere. The reaction mixture was quenched by adding water (30 mL) at 0° C., then filtered and the filter cake was dried in vacuo to give the title compound (400 mg, 798.75 μmol, 86.98% yield) as a white solid. ¹H NMR (DMSO-d₆, 400 MHz) δ 9.30 (s, 1H), 8.04 (s, 1H), 7.79 (d, J=1.2 Hz, 1H), 7.65 (d, J=1.2 Hz, 1H), 7.52 (d, J=8.0 Hz, 1H), 7.47 (d, J=2.0 Hz, 1H), 7.12 (d, J=8.4 Hz, 1H), 6.82 (d, J=8.8 Hz, 1H), 5.47 (d, J=6.8 Hz, 1H), 5.06 (s, 2H), 4.84-4.78 (m, 1H), 2.31 (s, 3H), 2.27-2.27 (m, 1H), 2.08 (s, 3H), 1.43 (d, J=6.8 Hz, 3H), 1.11-1.02 (m, 4H).

### Preparation of(S)-8-(1-((6-chloro-2-(1-hydroxy-1,3-dihydrobenzo[c][1,2]oxaborol-6-yl) pyridin-3-yl)amino)ethyl)-2-cyclopropyl-3,6-dimethyl-4H-chromen-4-one and (R)-8-(1-((6-chloro-2-(1-hydroxy-1,3-dihydrobenzo[c][1,2]oxaborol-6-yl)pyridin-3-yl)amino)ethyl)-2-cyclopropyl-3,6-dimethyl-4H-chromen-4-one

8-(1-((6-chloro-2-(1-hydroxy-1,3-dihydrobenzo[c][1,2]oxaborol-6-yl)pyridine-3-yl)amino)ethyl)-2-cyclopropyl-3,6-dimethyl-4H-chromen-4-one (800 mg, 1.60 mmol, 1 eq) was separated by SFC (column: ChiralPak IH, 250×30 mm×10 um; mobile phase: [CO2-MeOH (0.1% □H3H2O)]; B %: 47%, isocratic elution mode) to give the title compound, Isomer 1 (274.1 mg, 535.52 μmol, 33.52% yield) as a white solid and the title compound, Isomer 2 (253.4 mg, 501.61 μmol, 31.40% yield) as a white solid. Isomer 1: ¹H NMR (DMSO-d₆, 400 MHz) δ 9.30 (s, 1H), 8.04 (s, 1H), 7.81-7.79 (m, 1H), 7.66 (s, 1H), 7.52 (d, J=8.0 Hz, 1H), 7.47 (d, J=2.0 Hz, 1H), 7.12 (d, J=8.4 Hz, 1H), 6.82 (d, J=8.8 Hz, 1H), 5.48 (d, J=6.4 Hz, 1H), 5.06 (s, 2H), 4.84-4.78 (m, 1H), 2.31 (s, 3H), 2.29-2.25 (m, 1H), 2.08 (s, 3H), 1.43 (d, J=6.8 Hz, 3H), 1.11-1.03 (m, 4H). MS (ESI): mass calcd. For C₂₈H₂₆BClN₂O₄ 500.17, m/z found 501.2 [M+H]⁺. HPLC: 97.84% (220 nm), 98.61% (254 nm). Chiral purity: 99.82% ee. Isomer 2: ¹H NMR (DMSO-d₆, 400 MHz) δ 9.29 (s, 1H), 8.04 (s, 1H), 7.81-7.79 (m, 1H), 7.65 (d, J=1.6 Hz, 1H), 7.52 (d, J=8.0 Hz, 1H), 7.48 (d, J=2.0 Hz, 1H), 7.12 (d, J=8.8 Hz, 1H), 6.82 (d, J=8.8 Hz, 1H), 5.48 (d, J=7.2 Hz, 1H), 5.06 (s, 2H), 4.85-4.78 (m, 1H), 2.31 (s, 3H), 2.28-2.27 (m, 1H), 2.07 (s, 3H), 1.44 (d, J=6.4 Hz, 3H), 1.09-1.02 (m, 4H). MS (ESI): mass calcd. For C₂₈H₂₆BClN₂O₄ 500.17, m/z found 501.2 [M+H]⁺. HPLC: 99.14% (220 nm), 100% (254 nm). Chiral purity: 99.50% ee

### Example 111

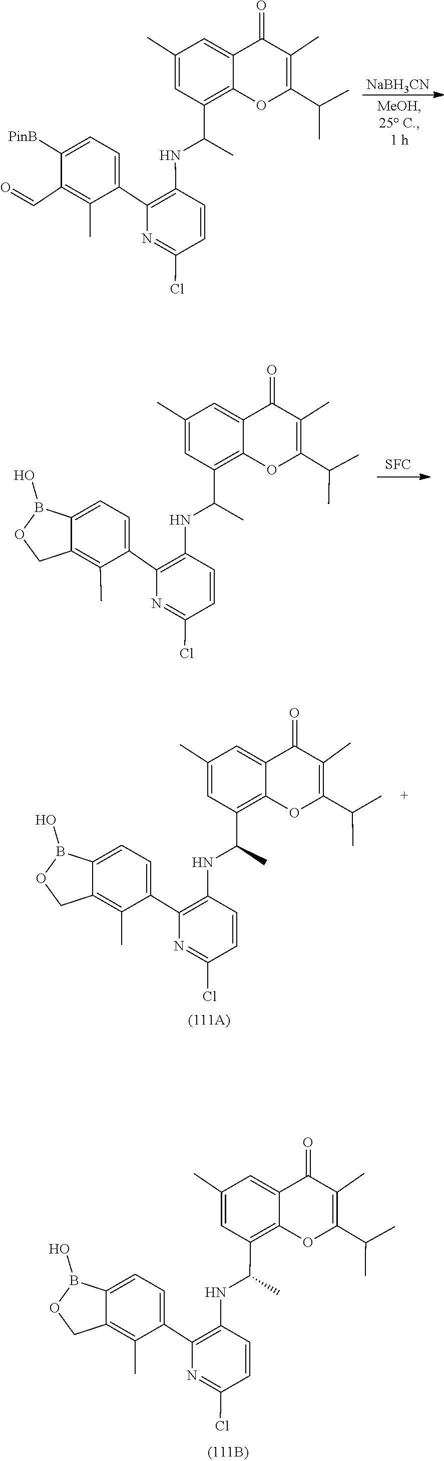

### Preparation of 8-[1-[[6-chloro-2-(1-hydroxy-4-methyl-3H-2,1-benzoxaborol-5-yl)-3-pyridyl]amino]ethyl]-2-isopropyl-3,6-dimethyl-chromen-4-one

To a solution of 3-[6-chloro-3-[1-(2-isopropyl-3,6-dimethyl-4-oxo-chromen-8-yl) ethylamino]-2-pyridyl]-2-methyl-6-(4,4,5,5-tetramethyl-1,3,2-dioxaborolan-2-yl)benzaldehyde (1.00 g, 1.63 mmol, 1 eq) in MeOH (10 mL) was added NaBH₃CN (102.18 mg, 1.63 mmol, 1 eq) at 25° C. in portions, the reaction mixture was stirred at 25° C. for 1 h. The reaction mixture was poured into H₂O (10 mL) and the resulting suspension was directly filtered. The filter cake was purified by prep-HPLC (column: Waters Xbridge BEH C18 250×50 mm×10 um; mobile phase: [H2O (10 mM □H4HCO3)-AC□]; gradient: 50%-90% B over 10.0 min) to give the title compound (150 mg, 290.24 μmol, 17.85% yield) as a white solid. ¹H NMR (DMSO-d₆, 400 MHz) δ 9.25 (s, 1H), 7.72-7.65 (m, 2H), 7.42 (s, 1H), 7.25 (d, J=7.6 Hz, 1H), 7.17 (d, J=8.4 Hz, 1H), 6.88 (d, J=8.0 Hz, 1H), 5.11-4.92 (m, 4H), 3.31-3.25 (m, 1H), 2.32 (s, 3H), 1.99 (s, 6H), 1.45 (d, J=6.4 Hz, 3H), 1.27 (d, J=6.8 Hz, 3H), 1.18 (d, J=6.8 Hz, 3H).

### Preparation of (S)-8-(1-((6-chloro-2-(1-hydroxy-4-methyl-1,3-dihydrobenzo[c][1,2]oxaborol-5-yl)pyridin-3-yl)amino)ethyl)-2-isopropyl-3,6-dimethyl-4H-chromen-4-one and (R)-8-(1-((6-chloro-2-(1-hydroxy-4-methyl-1,3-dihydrobenzo[c][1,2]oxaborol-5-yl)pyridin-3-yl)amino)ethyl)-2-isopropyl-3,6-dimethyl-4H-chromen-4-one

8-[1-[[6-chloro-2-(1-hydroxy-4-methyl-3H-2,1-benzoxaborol-5-yl)-3-pyridyl]amino]ethyl]-2-isopropyl-3,6-dimethyl-chromen-4-one (0.3 g, 580.47 gmol, 1 eq) was separated by SFC (column: DAICEL CHIRALCEL OD (250 mm×30 mm×10 um); mobile phase: [CO2-EtOH]; B %:27%, isocratic elution mode) to give the title compound, Isomer 1 (107.9 mg, 208.78 μmol, 35.97% yield) as a white solid and the title compound, Isomer 2 (133 mg, 257.34 μmol, 44.33% yield) as a white solid. Isomer 1: ¹H NMR (DMSO-d₆, 400 MHz) δ 9.24 (s, 1H), 7.74-7.60 (m, 2H), 7.42 (s, 1H), 7.25 (d, J=7.2 Hz, 1H), 7.17 (d, J=8.8 Hz, 1H), 6.89 (d, J=7.6 Hz, 1H), 5.08-5.00 (m, 3H), 4.98 (s, 1H), 3.30-3.21 (m, 1H), 2.32 (s, 3H), 1.99 (s, 6H), 1.45 (d, J=6.4 Hz, 3H), 1.27 (d, J=6.8 Hz, 3H), 1.18 (d, J=6.8 Hz, 3H). MS (ESI): mass calcd. For C₂₉H₃₀BClN₂O₄ 516.20, m/z found 517.2 [M+H]⁺. HPLC: 99.34% (220 nm), 99.50% (254 nm). Chiral purity: 100% ee. Isomer 2: ¹H NMR (DMSO-d₆, 400 MHz) δ 9.25 (s, 11H), 7.74-7.63 (m, 2H), 7.42 (s, 1H), 7.25 (d, J=7.2 Hz, 11H), 7.17 (d, J=8.8 Hz, 1H), 6.89 (d, J=7.2 Hz, 1H), 5.07-5.00 (m, 3H), 4.98 (s, 1H), 3.30-3.23 (m, 1H), 2.32 (s, 3H), 1.99 (s, 6H), 1.45 (d, J=6.4 Hz, 3H), 1.27 (d, J=7.2 Hz, 3H), 1.18 (d, J=6.8 Hz, 3H). MS (ESI): mass calcd. For C₂₉H₃₀BClN₂O₄ 516.20, m/z found 517.2 [M+H]⁺. HPLC: 98.97% (220 nm), 99.17% (254 nm). Chiral purity: 99.44% ee

### Example 112

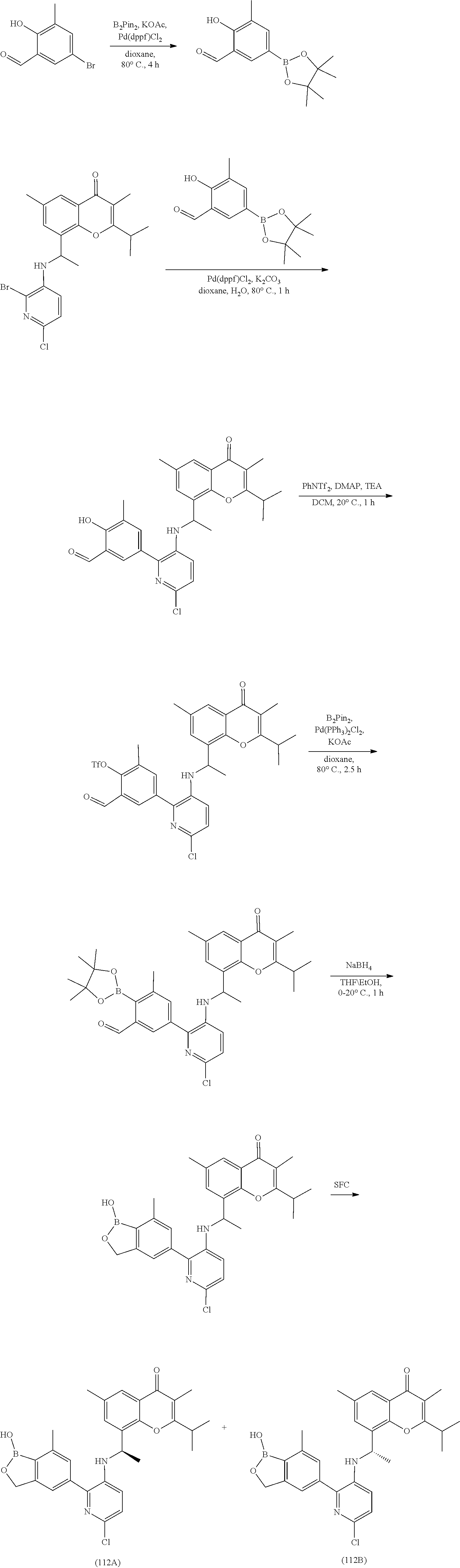

### Preparation of 2-hydroxy-3-methyl-5-(4,4,5,5-tetramethyl-1,3,2-dioxaborolan-2-yl) benzaldehyde

A mixture of 5-bromo-2-hydroxy-3-methyl-benzaldehyde (8.80 g, 40.92 mmol, 1 eq), B₂Pin₂ (20.78 g, 81.84 mmol, 2 eq), Pd(dppf)Cl₂ (2.99 g, 4.09 mmol, 0.1 eq) and KOAc (8.03 g, 81.84 mmol, 2 eq) in dioxane (100 mL) was degassed and purged with N₂ for 3 times at 20° C., and then the mixture was heated to 80° C. and stirred at 80° C. for 4 h under N₂ atmosphere. The reaction mixture was filtered and the filtrate was concentrated under reduced pressure. The residue was purified by flash silica gel chromatography (ISCO®; 220 g SepaFlash® Silica Flash Column, Eluent of 0~5% Ethyl acetate/Petroleum ether gradient @150 mL/min) to give the title compound (16.00 g, crude) as a yellow solid. ¹H NMR (DMSO-d₆, 400 MHz) δ 11.31 (s, 1H), 10.06 (s, 1H), 7.95 (s, 1H), 7.73 (s, 1H), 1.29 (s, 12H).

### Preparation of 5-[6-chloro-3-[1-(2-isopropyl-3,6-dimethyl-4-oxo-chromen-8-yl) ethylamino]-2-pyridyl]-2-hydroxy-3-methyl-benzaldehyde

The title compound was prepared as a yellow solid (3.8 g, 7.52 mmol, 84.61% yield) according to the procedures described herein and the synthetic scheme, using 8-[1-[(2-bromo-6-chloro-3-pyridyl)amino]ethyl]-2-isopropyl-3,6-dimethyl-chromen-4-one (4.00 g, 8.89 mmol, 1 eq) and 2-hydroxy-3-methyl-5-(4,4,5,5-tetramethyl-1,3,2-dioxaborolan-2-yl)benzaldehyde (3.50 g, 13.34 mmol, 1.5 eq). ¹H NMR (DMSO-d₆, 400 MHz) δ 11.16 (s, 1H), 10.10 (s, 1H), 7.99 (d, J=2.0 Hz, 1H), 7.81 (s, 1H), 7.68 (s, 1H), 7.56 (d, J=1.6 Hz, 1H), 7.12 (d, J=8.4 Hz, 1H), 6.86 (d, J=8.4 Hz, 1H), 5.72 (d, J=7.2 Hz, 1H), 5.03-4.95 (m, 1H), 3.29-3.25 (m, 1H), 2.34 (s, 3H), 2.26 (s, 3H), 1.99 (s, 3H), 1.52 (d, J=6.8 Hz, 3H), 1.24 (d, J=6.8 Hz, 3H), 1.18 (d, J=7.2 Hz, 3H).

### Preparation of [4-[6-chloro-3-[1-(2-isopropyl-3,6-dimethyl-4-oxo-chromen-8-yl) ethylamino]-2-pyridyl]-2-formyl-6-methyl-phenyl]trifluoromethanesulfonate

The title compound was prepared as a yellow solid (2.70 g, 4.24 mmol, 59.45% yield) according to the procedures described herein and the synthetic scheme, using 5-[6-chloro-3-[1-(2-isopropyl-3,6-dimethyl-4-oxo-chromen-8-yl) ethylamino]-2-pyridyl]-2-hydroxy-3-methyl-benzaldehyde (3.60 g, 7.13 mmol, 1 eq) and 1,1,1-trifluoro-N-phenyl-N-(trifluoromethylsulfonyl)methanesulfonamide (3.82 g, 10.69 mmol, 1.5 eq). ¹H NMR (CDCl₃, 400 MHz) δ 10.31 (s, 1H), 8.29 (d, J=2.0 Hz, 1H), 7.99 (d, J=2.0 Hz, 1H), 7.92 (d, J=1.2 Hz, 1H), 7.37 (d, J=2.0 Hz, 1H), 7.03 (d, J=8.8 Hz, 1H), 6.70 (d, J=8.4 Hz, 1H), 5.03-4.99 (m, 1H), 4.49 (s, 1H), 3.34-3.26 (m, 1H), 2.57 (s, 3H), 2.41 (s, 3H), 2.11 (s, 3H), 1.61 (d, J=6.4 Hz, 3H), 1.34 (d, J=6.8 Hz, 3H), 1.26 (d, J=6.8 Hz, 3H).

### Preparation of 5-(6-chloro-3-((1-(2-isopropyl-3,6-dimethyl-4-oxo-4H-chromen-8-yl)ethyl)amino)pyridin-2-yl)-3-methyl-2-(4,4,5,5-tetramethyl-1,3,2-dioxaborolan-2-yl)benzaldehyde

The title compound was prepared as a green solid (1.50 g, crude) according to the procedure described in preceding examples, using [4-[6-chloro-3-[1-(2-isopropyl-3,6-dimethyl-4-oxo-chromen-8-yl)ethylamino]-2-pyridyl]-2-formyl-6-methyl-phenyl]trifluoromethanesulfonate (1.30 g, 2.04 mmol, 1 eq) and B₂Pin₂ (1.04 g, 4.08 mmol, 2 eq). ¹H NMR (CDCl₃, 400 MHz) δ 10.06 (s, 1H), 7.99 (s, 1H), 7.90 (s, 1H), 7.76 (s, 1H), 7.28 (d, J=2.0 Hz, 1H), 7.00 (d, J=8.4 Hz, 1H), 6.62 (d, J=8.8 Hz, 1H), 5.03-4.95 (m, 1H), 4.44 (d, J=5.6 Hz, 1H), 3.34-3.27 (m, 1H), 2.57 (s, 3H), 2.39 (s, 3H), 2.11 (s, 3H), 1.52-1.51 (s, 15H), 1.35 (d, J=6.8 Hz, 3H), 1.30 (d, J=6.8 Hz, 3H).

### Preparation of 8-[1-[[6-chloro-2-(1-hydroxy-7-methyl-3H-2,1-benzoxaborol-5-yl)-3-pyridyl]amino]ethyl]-2-isopropyl-3,6-dimethyl-chromen-4-one

To a solution of 5-[6-chloro-3-[1-(2-isopropyl-3,6-dimethyl-4-oxo-chromen-8-yl)ethylamino]-2-pyridyl]-3-methyl-2-(4,4,5,5-tetramethyl-1,3,2-dioxaborolan-2-yl)benzaldehyde (1.00 g, 1.63 mmol, 1 eq) in THF (20 mL)/EtOH (2 mL) was added NaBH₄ (210 mg, 5.55 mmol, 3.41 eq) in portions at 20° C., the reaction mixture was stirred at 20° C. for 1 h. The reaction mixture was poured into H₂O (10 mL), adjusted to pH=5 by aqueous HCl (2N) and extracted with EtOAc (10 mL×3). The combined organic phase was dried with anhydrous Na₂SO₄, filtered and concentrated in vacuum. The residue was purified by flash silica gel chromatography (ISCO®; 12 g SepaFlash® Silica Flash Column, Eluent of 0~50% Ethyl acetate/Petroleum ether gradient @50 mE/min) and then further purified by prep-HPLC (column: 2_Phenomenex Gemini C18 75×40 mm×3 um; mobile phase: [H2O (10 mM NH4HCO3)-ACN]; gradient: 45%-70% B over 8.0 min) to give the title compound (270 mg, 522.42 mol, 32.13% yield) as a white solid. ¹H NMR (DMSO-d₆, 400 MHz) δ 9.01 (s, 1H), 7.69 (s, 1H), 7.55 (d, J=2.0 Hz, 1H), 7.48 (s, 1H), 7.39 (s, 1H), 7.14 (d, J=8.4 Hz, 1H), 6.90 (d, J=8.8 Hz, 1H), 5.53 (d, J=7.6 Hz, 1H), 5.01-4.96 (m, 3H), 3.30-3.25 (m, 1H), 2.49 (s, 3H), 2.34 (s, 3H), 1.98 (s, 3H), 1.51 (d, J=6.4 Hz, 3H), 1.22 (d, J=6.8 Hz, 3H), 1.14 (d, J=6.8 Hz, 3H).

### Preparation of 8-[(1S)-1-[[6-chloro-2-(1-hydroxy-7-methyl-3H-2,1-benzoxaborol-5-yl)-3-pyridyl]amino]ethyl]-2-isopropyl-3,6-dimethyl-chromen-4-one and 8-[(1R)-1-[[6-chloro-2-(1-hydroxy-7-methyl-3H-2,1-benzoxaborol-5-yl)-3-pyridyl]amino]ethyl]-2-isopropyl-3,6-dimethyl-chromen-4-one

8-[1-[[6-chloro-2-(1-hydroxy-7-methyl-3H-2,1-benzoxaborol-5-yl)-3-pyridyl]amino]ethyl]-2-isopropyl-3,6-dimethyl-chromen-4-one (550 mg, 1.06 mmol, 1 eq) was separated by SFC (column: DAICEL CHIRALCEL OD (250 mm×30 mm, 10 um); mobile phase: [CO2-MeOH]; B %:38%, isocratic elution mode) and then further purified by pre-HPLC (column: 2_Phenomenex Gemini C18 75×40 mm×3 um; mobile phase: [H₂O (10 mM NH₄HCO₃)-AC□]; gradient: 50%-80% B over 8.0 min) to give the title compound, Isomer 1 (159.7 mg, 309.00 μmol, 29.04% yield) as a white solid and the title compound, Isomer 2 (198.2 mg, 383.50 μmol, 36.04% yield) as a white solid. Isomer 1: ¹H NMR (DMSO-d₆, 400 MHz) δ 9.02 (s, 1H), 7.69 (d, J=1.2 Hz, 1H), 7.55 (d, J=2.0 Hz, 1H), 7.48 (s, 1H), 7.39 (s, 1H), 7.14 (d, J=8.4 Hz, 1H), 6.89 (d, J=8.8 Hz, 1H), 5.55 (d, J=7.2 Hz, 1H), 5.01-4.96 (m, 3H), 3.31-3.24 (m, 1H), 2.51-2.48 (m, 3H), 2.34 (s, 3H), 1.98 (s, 3H), 1.51 (d, J=6.4 Hz, 3H), 1.22 (d, J=6.4 Hz, 3H), 1.14 (d, J=6.8 Hz, 3H). MS (ESI): mass calcd. For C₂₉H₃₀BClN₂O₄ 516.20, m/z found 517.2 [M+H]⁺. HPLC: 99.57% (220 nm), 99.59% (254 nm). Chiral purity: 99.60% ee. Isomer 2: ¹H NMR (DMSO-d₆, 400 MHz) δ 9.02 (s, 1H), 7.69 (d, J=1.2 Hz, 1H), 7.55 (d, J=2.0 Hz, 1H), 7.48 (s, 1H), 7.39 (s, 1H), 7.14 (d, J=8.4 Hz, 1H), 6.89 (d, J=8.8 Hz, 1H), 5.55 (d, J=7.2 Hz, 1H), 5.01-4.96 (m, 3H), 3.31-3.24 (m, 1H), 2.51-2.48 (m, 3H), 2.34 (s, 3H), 1.98 (s, 3H), 1.51 (d, J=6.4 Hz, 3H), 1.22 (d, J=6.4 Hz, 3H), 1.14 (d, J=6.8 Hz, 3H). MS (ESI): mass calcd. For C₂₉H₃₀BClN₂O₄ 516.20, m/z found 517.2 [M+H]⁺. HPLC: 99.80% (220 nm), 99.69% (254 nm). Chiral purity: 100.00% ee.

### Example 113

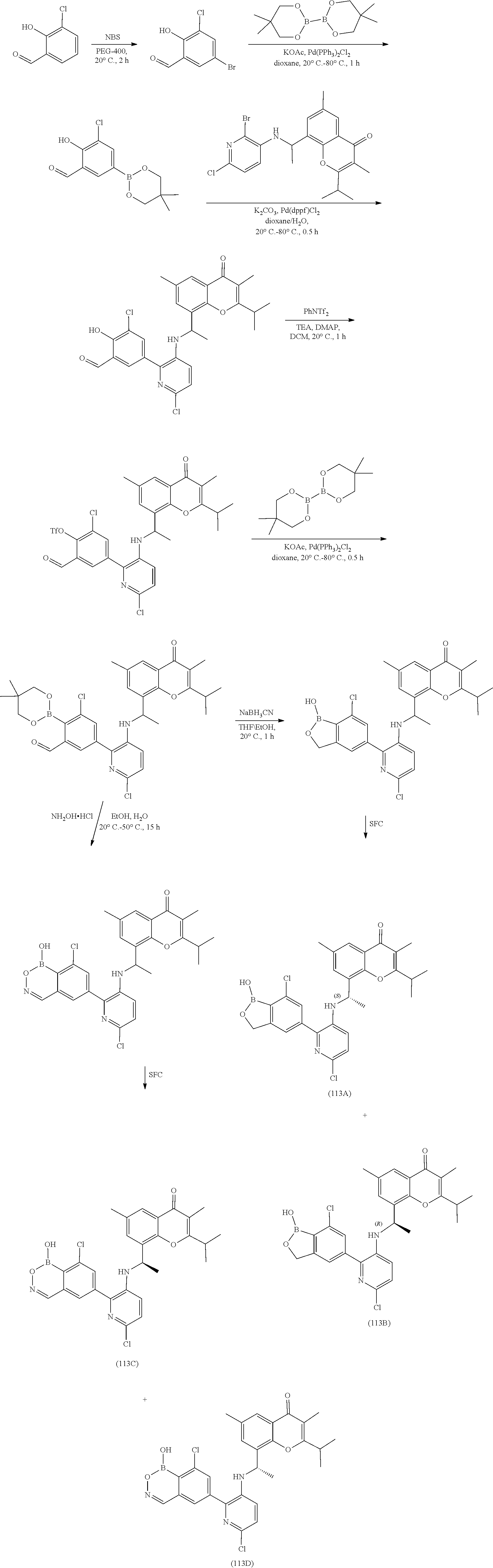

### Preparation of 5-bromo-3-chloro-2-hydroxy-benzaldehyde

To a solution of 3-chloro-2-hydroxy-benzaldehyde (20.0 g, 128 mmol, 1 eq) in PEG-400 (300 mL) was added NBS (23.9 g, 134 mmol, 1.05 eq) in portions at 20° C. under N₂, the reaction mixture was stirred at 20° C. for 2 h. The reaction mixture was poured into H₂O (1000 mL), and the resulting suspension was filtered. The filter cake was further purified by flash silica gel chromatography (ISCO®; 330 g SepaFlash® Silica Flash Column, Eluent of 0~2% Ethyl acetate/Petroleum ether gradient @100 mL/min) to give the title compound (27.0 g, 114.67 mmol, 89.77% yield) as a yellow solid. ¹H NMR (CDCl₃, 400 MHz) δ 11.40 (s, 1H), 9.86 (s, 1H), 7.75 (d, J=2.4 Hz, 1H), 7.63 (d, J=2.4 Hz, 1H).

### Preparation of 3-chloro-5-(5,5-dimethyl-1,3,2-dioxaborinan-2-yl)-2-hydroxy-benzaldehyde

To a mixture of 5-bromo-3-chloro-2-hydroxy-benzaldehyde (8.00 g, 34.0 mmol, 1 eq) and 2-(5,5-dimethyl-1,3,2-dioxaborinan-2-yl)-5,5-dimethyl-1,3,2-dioxaborinane (9.98 g, 44.2 mmol, 1.3 eq) in dioxane (120 mL) were added Pd(PPh₃)₂Cl2 (2.38 g, 3.40 mmol, 0.1 eq) and KOAc (6.67 g, 68.0 mmol, 2 eq) at 20° C., the reaction mixture was heated to 80° C. and stirred for 1 h. The reaction mixture was cooled to 20° C., filtered and the filtrate was concentrated under reduced pressure to give a residue. The residue was purified by flash silica gel chromatography (Biotage®; 120 g SepaFlash® Silica Flash Column, Eluent of 0~10% Ethyl acetate/Petroleum ether gradient @80 mL/min) to give the tide compound (4.00 g, 14.90 mmol, 43.85% yield) as a white solid. ¹H NMR (CDCl₃, 400 MHz) δ 11.67 (s, 1H), 9.92 (s, 1H), 8.03 (s, 1H), 7.95 (s, 1H), 3.78 (s, 4H), 1.04 (s, 6H).

### Preparation of 3-chloro-5-[6-chloro-3-[1-(2-isopropyl-3,6-dimethyl-4-oxo-chromen-8-yl)ethylamino]-2-pyridyl]-2-hydroxy-benzaldehyde

The title compound was prepared as a yellow solid (2.30 g, 4.38 mmol, 61.53% yield) according to the procedures described herein and the synthetic scheme, using 8-[1-[(2-bromo-6-chloro-3-pyridyl)amino]ethyl]-2-isopropyl-3,6-dimethyl-chromen-4-one (3.20 g, 7.11 mmol, 1 eq) and 3-chloro-5-(5,5-dimethyl-1,3,2-dioxaborinan-2-yl)-2-hydroxy-benzaldehyde (2.48 g, 9.25 mmol, 1.3 eq). ¹H NMR (CDCl₃, 400 MHz) δ 11.65 (s, 1H), 10.00 (s, 1H), 8.06 (d, J=2.0 Hz, 1H), 7.93 (d, J=2.0 Hz, 1H), 7.92 (s, 1H), 7.32 (d, J=2.0 Hz, 1H), 7.01 (d, J=8.8 Hz, 1H), 6.68 (d, J=8.4 Hz, 1H), 5.05-4.93 (m, 1H), 4.60-4.15 (m, 1H), 3.38-3.24 (m, 1H), 2.40 (s, 3H), 2.11 (s, 3H), 1.60 (d, J=6.8 Hz, 3H), 1.35 (d, J=6.8 Hz, 3H), 1.27 (d, J=6.8 Hz, 3H).

### Preparation of [2-chloro-4-[6-chloro-3-[1-(2-isopropyl-3,6-dimethyl-4-oxo-chromen-8-yl)ethylamino]-2-pyridyl]-6-formyl-phenyl]trifluoromethanesulfonate

The title compound was prepared as a yellow solid (4.00 g, 6.08 mmol, 83.68% yield) according to the procedures described herein and the synthetic scheme, using 3-chloro-5-[6-chloro-3-[1-(2-isopropyl-3,6-dimethyl-4-oxo-chromen-8-yl)ethylamino]-2-pyridyl]-2-hydroxy-benzaldehyde (3.82 g, 7.27 mmol, 1 eq) and PhNTf₂ (2.60 g, 7.27 mmol, 1 eq). ¹H NMR (CDCl₃, 400 MHz) δ 10.30 (s, 1H), 8.38 (d, J=2.0 Hz, 1H), 8.23 (d, J=2.0 Hz, 1H), 7.93 (s, 1H), 7.37 (d, J=2.0 Hz, 1H), 7.05 (d, J=8.4 Hz, 1H), 6.73 (d, J=8.8 Hz, 1H), 5.08-4.92 (m, 1H), 4.46 (br s, 1H), 3.40-3.22 (m, 1H), 2.42 (s, 3H), 2.11 (s, 3H), 1.63 (d, J=6.8 Hz, 3H), 1.34 (d, J=6.8 Hz, 3H), 1.29-1.26 (m, 3H).

### Preparation of 3-chloro-5-[6-chloro-3-[1-(2-isopropyl-3,6-dimethyl-4-oxo-chromen-8-yl)ethylamino]-2-pyridyl]-2-(5,5-dimethyl-1,3,2-dioxaborinan-2-yl)benzaldehyde

The title compound was prepared as a yellow solid (2.70 g, 4.35 mmol, 95.23% yield) according to the procedures described herein and the synthetic scheme, using [2-chloro-4-[6-chloro-3-[1-(2-isopropyl-3,6-dimethyl-4-oxo-chromen-8-yl)ethylamino]-2-pyridyl]-6-formyl-phenyl]trifluoromethanesulfonate (3.00 g, 4.56 mmol, 1 eq) and 2-(5,5-dimethyl-1,3,2-dioxaborinan-2-yl)-5,5-dimethyl-1,3,2-dioxaborinane (1.34 g, 5.93 mmol, 1.3 eq). ¹H NMR (CDCl₃, 400 MHz) δ 10.03 (s, 1H), 8.06 (s, 1H), 7.97 (s, 1H), 7.91 (s, 1H), 7.30 (d, J=2.0 Hz, 1H), 7.02 (d, J=8.8 Hz, 1H), 6.66 (d, J=8.8 Hz, 1H), 5.05-4.94 (m, 1H), 4.42 (d, J=5.2 Hz, 1H), 3.89 (s, 4H), 3.36-3.25 (m, 1H), 2.40 (s, 3H), 2.11 (s, 3H), 1.56 (d, J=6.4 Hz, 3H), 1.34 (d, J=6.8 Hz, 3H), 1.28 (d, J=6.8 Hz, 3H), 1.21 (s, 6H).

### Preparation of 8-[1-[[6-chloro-2-(7-chloro-1-hydroxy-3H-2,1-benzoxaborol-5-yl)-3-pyridyl]amino]ethyl]-2-isopropyl-3,6-dimethyl-chromen-4-one

To a solution of 3-chloro-5-[6-chloro-3-[1-(2-isopropyl-3,6-dimethyl-4-oxo-chromen-8-yl) ethylamino]-2-pyridyl]-2-(5,5-dimethyl-1,3,2-dioxaborinan-2-yl)benzaldehyde (1.50 g, 2.41 mmol, 1 eq) in THF (20 mL)/EtOH (2 mL) was added NaBH₃CN (227.56 mg, 3.62 mmol, 1.5 eq) in portions at 0° C., the reaction mixture was stirred at 20° C. for 1 h. The reaction mixture was poured into H₂O (40 mL) at 0° C. and the resulting suspension was filtered to give the title compound (0.83 g, 1.54 mmol, 64.00% yield) as a yellow solid. ¹H NMR (DMSO-d₆, 400 MHz) δ 9.24 (s, 1H), 7.69 (s, 2H), 7.62 (s, 1H) 7.55 (d, J=2.0 Hz, 1H), 7.17 (d, J=8.8 Hz, 1H), 6.90 (d, J=8.8 Hz, 1H), 5.83 (d, J=7.2 Hz, 1H), 5.10-5.03 (m, 2H), 5.00 (t, J=7.2 Hz, 1H), 3.29-3.26 (m, 1H), 2.34 (s, 3H), 1.99 (s, 3H), 1.51 (d, J=6.8 Hz, 3H), 1.24 (d, J=6.8 Hz, 3H), 1.19 (d, J=6.8 Hz, 3H).

### Preparation of (S)-8-(1-((6-chloro-2-(7-chloro-1-hydroxy-1,3-dihydrobenzo[c][1,2]oxaborol-5-yl)pyridin-3-yl)amino)ethyl)-2-isopropyl-3,6-dimethyl-4H-chromen-4-one and (R)-8-(1-((6-chloro-2-(7-chloro-1-hydroxy-1,3-dihydrobenzo[c][1,2]oxaborol-5-yl) pyridin-3-yl)amino)ethyl)-2-isopropyl-3,6-dimethyl-4H-chromen-4-one

8-[1-[[6-chloro-2-(7-chloro-1-hydroxy-3H-2,1-benzoxaborol-5-yl)-3-pyridyl]amino]ethyl]-2-isopropyl-3,6-dimethyl-chromen-4-one (800 mg) was further separated by SFC (column: DAICEL CHIRALCEL OD (250 mm×30 mm×10 um); mobile phase: [CO₂-EtOH]; B %:36%, isocratic elution mode) and purified by prep-HPLC (column: Waters Xbridge BEH C18 250×50 mm×10 um; mobile phase: [H₂O (10 mM NH₄HCO₃)-AC□]; gradient: 35%-65% B over 10.0 min) to give the title compound, Isomer 1 (189.8 mg, 352.26 μmol, 23.66% yield) as a white solid and the title compound, Isomer 2 (153.2 mg, 284.90 μmol, 19.13% yield) as a white solid. Isomer 1: ¹H NMR (DMSO-d₆, 400 MHz) δ 9.24 (s, 1H), 7.69 (s, 2H), 7.62 (s, 1H), 7.56 (s, 1H), 7.17 (d, J=8.8 Hz, 1H), 6.89 (d, J=8.8 Hz, 1H), 5.84 (d, J=7.2 Hz, 1H), 5.09-5.02 (m, 2H), 5.00 (t, J=6.8 Hz, 1H), 3.30-3.25 (m, 1H), 2.34 (s, 3H), 1.99 (s, 3H), 1.51 (d, J=6.8 Hz, 3H), 1.24 (d, J=6.8 Hz, 3H), 1.18 (d, J=6.8 Hz, 3H). MS (ESI): mass calcd. For C₂₈H₂₇BCl₂N₂O₄ 536.14, m/z found 537.1 [M+H]⁺. HPLC: 99.70% (220 nm), 99.91% (254 nm). Chiral purity: 97.4% ee. Isomer 2: ¹H NMR (DMSO-d₆, 400 MHz) δ 9.24 (s, 1H), 7.69 (s, 2H), 7.62 (s, 1H), 7.56 (d, J=2.0 Hz, 1H), 7.17 (d, J=8.4 Hz, 1H), 6.89 (d, J=8.8 Hz, 1H), 5.84 (d, J=7.2 Hz, 1H), 5.10-5.03 (m, 2H), 5.00 (t, J=7.2 Hz, 1H), 3.29-3.26 (m, 1H), 2.34 (s, 3H), 1.99 (s, 3H), 1.51 (d, J=6.4 Hz, 3H), 1.24 (d, J=6.8 Hz, 3H), 1.19 (d, J=7.2 Hz, 3H) MS (ESI): mass calcd. For C₂₈H₂₇BCl₂N₂O₄ 536.14, m/z found 537.2 [M+H]⁺. HPLC: 99.91% (220 nm), 100.00% (254 nm). Chiral purity: 100% ee

### Preparation of 8-(1-((6-chloro-2-(8-chloro-1-hydroxy-1H-benzo[d][1,2,6]oxazahorinin-6-yl)pyridin-3-yl)amino)ethyl)-2-isopropyl-3,6-dimethyl-4H-chromen-4-one

The title compound was prepared as a pale-yellow solid (0.7 g, 1.23 mmol, 56.58% yield) according to the procedures described herein and the synthetic scheme, using 3-chloro-5-[6-chloro-3-[l-(2-isopropyl-3,6-dimethyl-4-oxo-chromen-8-yl)ethylamino]-2-pyridyl]-2-(5,5-dimethyl-1,3,2-dioxaborinan-2-yl)benzaldehyde (2.0 g, 3.22 mmol, 1 eq) and NH₂OH·HCl (336 mg, 4.82 mmol, 1.5 eq). ¹H NMR (DMSO-d₆, 400 MHz) δ 8.67 (s, 1H), 7.99 (d, J=6.4 Hz, 2H), 7.69 (s, 1H), 7.56 (d, J=2.0 Hz, 1H), 7.20 (d, J=8.4 Hz, 1H), 6.91 (d, J=8.8 Hz, 1H), 6.07 (d, J=7.2 Hz, 1H), 5.02-4.98 (m, 1H), 3.30-3.25 (m, 1H), 2.34 (s, 3H), 1.99 (s, 3H), 1.52 (d, J=6.8 Hz, 3H), 1.24 (d, J=6.8 Hz, 3H), 1.21 (d, J=6.8 Hz, 3H).

### Preparation of (S)-8-(1-((6-chloro-2-(8-chloro-1-hydroxy-JH-benzo[d][1,2,6]oxazaborinin-6-yl)pyridin-3-yl)amino)ethyl)-2-isopropyl-3,6-dimethyl-4H-chromen-4-one and (R)-8-(1-((6-chloro-2-(8-chloro-1-hydroxy-JH-benzo[d][1,2,6]oxazaborinin-6-yl)pyridin-3-yl)amino)ethyl)-2-isopropyl-3,6-dimethyl-4H-chromen-4-one

The product 8-(1-((6-chloro-2-(8-chloro-1-hydroxy-1H-benzo[d][1,2,6]oxazaborinin-6-yl) pyridin-3-yl)amino)ethyl)-2-isopropyl-3,6-dimethyl-4H-chromen-4-one (0.9 g) was further separated by SFC (column: DAICEL CHIRALPAK IK (250 mm×25 mm×10 um); mobile phase: [CO₂-EtOH]; B %:55%, isocratic elution mode) and purified by prep-HPLC (column: 2_Phenomenex Gemini C18 75×40 mm×3 um; mobile phase: [H₂O (10 mM NH₄HCO₃)-AC□]; gradient: 15%-45% B over 8.0 min) to give the title compound, Isomer 1 (197.6 mg, 355.92 gmol, 22.43% yield) as a white solid and the title compound, Isomer 2 (120.4 mg, 217.00 μmol, 13.68% yield) as an off-white solid. Isomer 1: ¹H NMR (DMSO-d₆, 400 MHz) δ 8.64 (s, 1H), 7.98 (d, J=4.8 Hz, 2H), 7.69 (s, 1H), 7.56 (d, J=2.0 Hz, 1H), 7.20 (d, J=8.4 Hz, 1H), 6.91 (d, J=9.2 Hz, 1H), 6.05 (d, J=6.8 Hz, 1H), 5.05-4.96 (m, 1H), 3.29-3.23 (m, 1H), 2.34 (s, 3H), 1.99 (s, 3H), 1.52 (d, J=6.8 Hz, 3H), 1.24 (d, J=6.8 Hz, 3H), 1.21 (d, J=6.8 Hz, 3H). MS (ESI): mass calcd. For C₂₈H₂₆BCl₂N₃O₄ 549.14, m/z found 550.0 [M+H]⁺. HPLC: 99.11% (220 nm), 99.20% (254 nm). Chiral purity: 100% ee. Isomer 2: ¹H NMR (DMSO-d₆, 400 MHz) δ 9.40 (s, 1H), 8.75 (s, 1H), 8.06 (s, 1H), 8.03 (s, 1H), 7.68 (s, 1H) 7.56 (d, J=2.0 Hz, 1H), 7.21 (d, J=8.4 Hz, 1H), 6.91 (d, J=8.8 Hz, 1H), 6.13 (d, J=7.2 Hz, 1H), 5.07-4.94 (m, 1H), 3.30-3.25 (m, 1H), 2.34 (s, 3H), 1.99 (s, 3H), 1.52 (d, J=6.8 Hz, 3H), 1.24 (d, J=6.8 Hz, 3H), 1.20 (d, J=6.8 Hz, 3H). MS (ESI): mass calcd. For C28H₂₆BCl₂N₃O₄ 549.14, m/z found 550.2 [M+H]⁺. HPLC: 99.08% (220 nm), 99.04% (254 nm). Chiral purity: 100% ee

### Example 114

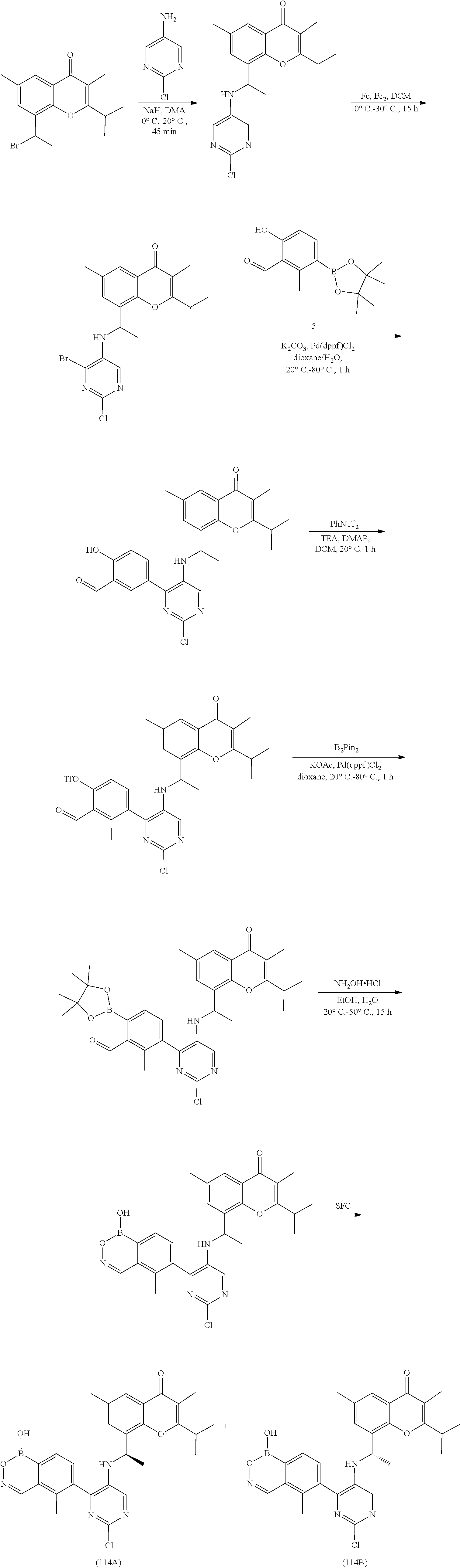

### Preparation of 8-(1-((2-chloropyrimidin-5-yl)amino)ethyl)-2-isopropyl-3,6-dimethyl-4H-chromen-4-one

The title compound was prepared as a yellow solid (10.50 g, 28.24 mmol, 60.84% yield) according to the procedures described herein and the synthetic scheme, using 2-chloropyrimidin-5-amine (6.00 g, 46.41 mmol, 1 eq) and 8-(1-bromoethyl)-2-iso propyl-3,6-dimethyl-4H-chromen-4-one (15.0 g, 46.4 mmol, 1 eq). ¹H NMR (CDCl₃, 400 MHz) δ 7.91 (s, 1H), 7.89 (s, 2H), 7.38 (d, J=2.0 Hz, 1H), 5.10-4.98 (m, 1H), 4.34 (d, J=6.0 Hz, 1H), 3.38-3.27 (m, 1H), 2.38 (s, 3H), 2.11 (s, 3H), 1.67 (d, J=6.8 Hz, 3H), 1.36 (dd, J=6.8, 2.4 Hz, 6H).

### Preparation of 8-(1-((4-bromo-2-chloropyrimidin-5-yl)amino)ethyl)-2-isopropyl-3,6-dimethyl-4H-chromen-4-one

To a mixture 8-(1-((2-chloropyrimidin-5-yl)amino)ethyl)-2-isopropyl-3,6-dimethyl-4H-chromen-4-one (1.20 g, 3.23 mmol, 1 eq) and Fe (36 mg, 645 μmol, 0.2 eq) in DCM (15 mL) was added Br₂ (619 mg, 3.87 mmol, 199 μL, 1.2 eq) dropwise at 0° C., the reaction mixture was heated to 30° C. and stirred for 15 h. The reaction mixture was diluted with H₂O (20 mL) and extracted with DCM (15 mL×3). The combined organic layers were washed with sat.aq. Na₂SO₃ (15 mL), then brine (20 mL×3), dried over Na₂SO₄, filtered and concentrated under reduced pressure to give a residue. The residue was purified by flash silica gel chromatography (Biotage®; 20 g SepaFlash® Silica Flash Column, Eluent of 0-25% Ethyl acetate/Petroleum ether gradient @40 mL/min) to give the title compound (440 mg, 976.14 mol, 30.25% yield) as a yellow solid. ¹H NMR (CDCl₃, 400 MHz) δ 7.93 (s, 1H), 7.50 (s, 1H), 7.33 (d, J=2.0 Hz, 1H), 5.15-5.02 (m, 1H), 4.77 (d, J=5.6 Hz, 1H), 3.39-3.22 (m, 1H), 2.40 (s, 3H), 2.11 (s, 3H), 1.73 (d, J=6.8 Hz, 3H), 1.37 (dd, J=6.8, 2.8 Hz, 6H)

### Preparation of 3-(2-chloro-5-((1-(2-isopropyl-3,6-dimethyl-4-oxo-4H-chromen-8-yl)ethyl)amino)pyrimidin-4-yl)-6-hydroxy-2-methylbenzaldehyde

The title compound was prepared as a yellow solid (445 mg, 879.46 μmol, 99.11% yield) according to the procedure described in preceding examples, using 8-(1-((4-bromo-2-chloropyrimidin-5-yl)amino)ethyl)-2-isopropyl-3,6-dimethyl-4H-chromen-4-one (400 mg, 887 μmol, 1 eq) and 6-hydroxy-2-methyl-3-(4,4,5,5-tetramethyl-1,3,2-dioxaborolan-2-yl)benzaldehyde (279 mg, 1.06 mmol, 1.2 eq). ¹H NMR (CDCl₃, 400 MHz) δ 12.29 (s, 1H), 10.48 (s, 1H), 7.92 (s, 1H), 7.76 (s, 1H), 7.47 (d, J=8.4 Hz, 1H), 7.22 (br s, 1H), 7.04 (d, J=8.8 Hz, 1H), 5.15-5.00 (m, 1H), 3.98 (d, J=4.8 Hz, 1H), 3.40-3.20 (m, 1H), 2.53 (s, 3H), 2.38 (s, 3H), 2.11 (s, 3H), 1.55 (d, J=6.4 Hz, 3H), 1.35 (d, J=7.2 Hz, 3H), 1.28 (d, J=6.8 Hz, 3H).

### Preparation of 4-(2-chloro-5-((1-(2-isopropyl-3,6-dimethyl-4-oxo-4H-chromen-8-yl)ethyl)amino)pyrimidin-4-yl)-2-formyl-3-methylphenyltrifluoromethanesulfonate

The title compound was prepared as a yellow solid (450 mg, 705.27 μmol, 89.22% yield) according to the procedures described herein and the synthetic scheme, using 3-(2-chloro-5-((1-(2-isopropyl-3,6-dimethyl-4-oxo-4H-chromen-8-yl)ethyl)amino)pyrimidin-4-yl)-6-hydroxy-2-methylbenzaldehyde (400 mg, 791 μmol, 1 eq) and PhNTf₂ (283 mg, 790.53 μmol, 1 eq). ¹H NMR (CDCl₃, 400 MHz) δ 10.58 (s, 1H), 7.92 (s, 1H), 7.81 (d, J=12.0 Hz, 1H), 7.65 (d, J=8.4 Hz, 1H), 7.59 (d, J=8.8 Hz, 1H), 7.21 (br s, 1H) 5.17-5.05 (m, 1H), 3.87 (br s, 1H), 3.40-3.20 (m, 1H), 2.54 (s, 3H), 2.38 (s, 3H), 2.11 (s, 3H), 1.58 (s, 3H), 1.35 (d, J=6.8 Hz, 3H), 1.29-1.25 (m, 3H).

### Preparation of 3-(2-chloro-5-((1-(2-isopropyl-3,6-dimethyl-4-oxo-4H-chromen-8-yl) ethyl)amino)pyrimidin-4-yl)-2-methyl-6-(4,4,5,5-tetramethyl-1,3,2-dioxaborolan-2-yl)benzaldehyde

The title compound was prepared as a yellow solid (320 mg, 519.52 μmol, 82.87% yield) according to the procedures described herein and the synthetic scheme, using 4-(2-chloro-5-((1-(2-isopropyl-3,6-dimethyl-4-oxo-4H-chromen-8-yl)ethyl)amino)pyrimidin-4-yl)-2-formyl-3-methylphenyl trifluoromethanesulfonate (400 mg, 627 gmol, 1 eq) and B₂pin₂ (318 mg, 1.25 mmol, 2 eq). ¹H NMR (CDCl₃, 400 MHz) δ10.62 (s, 1H), 7.91 (s, 1H), 7.78-7.65 (m, 2H), 7.52 (d, J=7.6 Hz, 1H), 7.23-7.10 (m, 1H), 5.15-5.02 (m, 1H), 3.83 (d, J=5.2 Hz, 1H), 3.37-3.25 (m, 1H), 2.54 (s, 3H), 2.37 (s, 3H), 2.11 (s, 3H), 1.48 (d, J=6.8 Hz, 3H), 1.46 (s, 12H), 1.36 (d, J=6.8 Hz, 3H), 1.29 (d, J=6.8 Hz, 3H).

### Preparation of 8-(1-((2-chloro-4-(1-hydroxy-5-methyl-JH-benzo[d][1,2,6]oxazaborinin-6-yl)pyrimidin-5-yl)amino)ethyl)-2-isopropyl-3,6-dimethyl-4H-chromen-4-one

The title compound was prepared as a white solid (110 mg, 207.23 μmol, 51.06% yield) according to the procedures described herein and the synthetic scheme, using 3-(2-chloro-5-((1-(2-isopropyl-3,6-dimethyl-4-oxo-4H-chromen-8-yl)ethyl)amino)pyrimidin-4-yl)-2-methyl-6-(4,4,5,5-tetramethyl-1,3,2-dioxaborolan-2-yl)benzaldehyde (250 mg, 406 μmol, 1 eq) and NH₂OH·HCl (34 mg, 487.05 μmol, 1.2 eq). ¹H NMR (DMSO-d₆, 400 MHz) δ 9.42 (s, 1H), 9.00-8.75 (m, 2H), 8.01 (d, J=7.6 Hz, 1H), 7.99-7.87 (m, 1H), 7.75-7.54 (m, 1H), 7.51-7.30 (m, 1H), 5.90-5.60 (m, 1H), 5.17-5.02 (m, 1H), 3.31-3.24 (m, 1H), 2.34 (s, 6H), 1.99 (s, 3H), 1.45 (d, J=6.8 Hz, 3H), 1.27 (d, J=6.8 Hz, 3H), 1.21 (d, J=6.8 Hz, 3H).

### Preparation of (S)-8-(1-((2-chloro-4-(1-hydroxy-5-methyl-JH-benzo[d][1,2,6]oxazaborinin-6-yl)pyrimidin-5-yl)amino)ethyl)-2-isopropyl-3,6-dimethyl-4H-chromen-4-one and (R)-8-(1-((2-chloro-4-(1-hydroxy-5-methyl-JH-benzo[d][1,2,6]oxazaborinin-6-yl)pyrimidin-5-yl)amino)ethyl)-2-isopropyl-3,6-dimethyl-4H-chromen-4-one

The product 8-(1-((2-chloro-4-(1-hydroxy-5-methyl-1H-benzo[d][1,2,6]oxazaborinin-6-yl) pyrimidin-5-yl)amino)ethyl)-2-isopropyl-3,6-dimethyl-4H-chromen-4-one (90 mg) was further separated by SFC (column: DAICEL CHIRALPAK IK (250 mm×25 mm×10 um); mobile phase: [CO₂-EtOH]; B %:55%, isocratic elution mode) to give the title compound, Isomer 1 (38.9 mg, 73.14 μmol, 44.02% yield) as a white solid and the title compound, Isomer 2 (36.4 mg, 68.25 μmol, 41.07% yield) as a white solid. Isomer 1: ¹H NMR (DMSO-d₆, 400 MHz) δ 9.45 (s, 1H), 9.00-8.77 (m, 2H), 8.01 (d, J=7.6 Hz, 1H), 7.99-7.87 (m, 1H), 7.71-7.54 (m, 1H), 7.52-7.36 (m, 1H), 5.90-5.65 (m, 1H), 5.20-5.49 (m, 1H), 3.30-3.23 (m, 1H), 2.34 (s, 6H), 1.99 (s, 3H), 1.45 (d, J=6.4 Hz, 3H), 1.27 (d, J=6.8 Hz, 3H), 1.21 (d, J=6.8 Hz, 3H). MS (ESI): mass calcd. For C₂₈H₂₈BClN₄O₄ 530.19, m/z found 531.1 [M+H]⁺. HPLC: 99.80% (220 nm), 99.90% (254 nm). Chiral purity: 97.4% ee. Isomer 2: ¹H NMR (DMSO-d₆, 400 MHz) δ 9.45 (s, 1H), 9.00-8.77 (m, 2H), 8.01 (d, J=7.6 Hz, 1H), 7.99-7.87 (m, 1H), 7.73-7.55 (m, 1H), 7.51-7.34 (m, 1H), 5.85-5.62 (m, 1H), 5.15-5.05 (m, JH), 3.30-3.24 (m, 1H), 2.34 (s, 6H), 1.99 (s, 3H), 1.45 (d, J=6.4 Hz, 3H), 1.27 (d, J=6.8 Hz, 3H), 1.21 (d, J=6.8 Hz, 3H) MS (ESI): mass calcd. For C₂₈H₂₈BClN₄O₄ 530.19, m/z found 531.1 [M+H]⁺. HPLC: 99.52% (220 nm), 99.64% (254 nm). Chiral purity: 99.54% ee

### Example 115

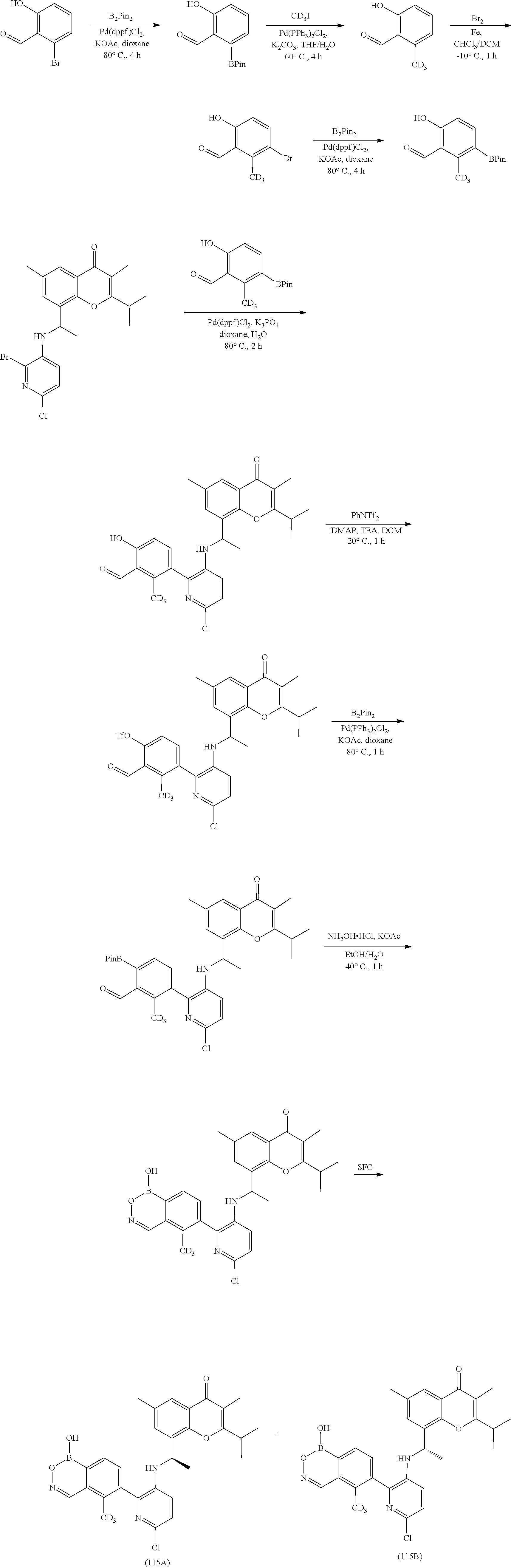

### Preparation of 2-hydroxy-6-(4,4,5,5-tetramethyl-1,3,2-dioxaborolan-2-yl)benzaldehyde

To a solution of 2-bromo-6-hydroxy-benzaldehyde (10.0 g, 49.7 mmol, 1 eq) in dioxane (150 mL) were added B₂Pin₂ (18.9 g, 74.6 mmol, 1.5 eq), Pd(dppf)Cl₂ (2.9 g, 3.98 mmol, 0.08 eq) and KOAc (12.2 g, 124 mmol, 2.5 eq) in turns at 20° C. The reaction mixture was degassed and purged with N₂ for 3 times, and then the mixture was directly moved to the oil-bath (pre-heated to 80° C.) and stirred at 80° C. for 4 h under N₂ atmosphere. After cooling to room temperature, the reaction mixture was filtered and the filtrate was concentrated under reduced pressure to give a residue. The residue was purified by flash silica gel chromatography (Biotage®; 220 g SepaFlash® Silica Flash Column, Eluent of 0~10% Ethyl acetate/Petroleum ether gradient @100 mL/min) to give the title compound (9.5 g, 38.29 mmol, 76.98% yield) as a yellow solid. ¹H NMR (DMSO-d₆, 400 MHz) δ 11.01 (s, 1H), 10.37 (s, 1H), 7.52-7.47 (m, 1H), 7.04-7.00 (m, 2H), 1.32 (s, 12H).

### Preparation of 2-hydroxy-6-(trideuteriomethyl)benzaldehyde

To a solution of 2-hydroxy-6-(4,4,5,5-tetramethyl-1,3,2-dioxaborolan-2-yl)benzaldehyde (9.20 g, 37.1 mmol, 1 eq) in THF (100 mL)/H₂O (10 mL) were added trideuterio(iodo)methane (6.45 g, 44.5 mmol, 2.77 mL, 1.2 eq), Pd(PPh₃)₂Cl₂ (1.30 g, 1.85 mmol, 0.05 eq) and K2CO₃ (12.8 g, 92.7 mmol, 2.5 eq) in turns at 20° C. The reaction mixture was degassed and purged with N₂ for 3 times, and then the mixture was directly moved to the oil-bath (pre-heated to 60° C.) and stirred at 60° C. for 4 h under N₂ atmosphere. After cooling to room temperature, the reaction mixture was poured into H₂O (200 mL) and extracted with EtOAc (200 mL×3). The combined organic layers were washed with brine (200 mL×2), dried over Na₂SO₄, filtered and the filtrate was concentrated under reduced pressure to give a residue. The residue was purified by flash silica gel chromatography (Biotage®; 80 g SepaFlash® Silica Flash Column, Eluent of 0~10% Ethyl acetate/Petroleum ether gradient @80 mL/min) to give the title compound (3.60 g, 25.87 mmol, 69.76% yield) as a yellow solid. ¹H NMR (CDCl₃, 400 MHz) δ 11.91 (s, 1H), 10.33 (s, 1H), 7.39 (t, J=7.6 Hz, 1H), 6.83 (d, J=8.4 Hz, 1H), 6.72 (dd, J=7.6, 1.2 Hz, 1H).

### Preparation of 3-bromo-6-hydroxy-2-(trideuteriomethyl)benzaldehyde

To a solution of 2-hydroxy-6-(trideuteriomethyl)benzaldehyde (3.60 g, 25.9 mmol, 1 eq) in CHCl₃ (40 mL)/DCM (4 mL) was added Fe (72 mg, 1.29 mmol, 0.05 eq) in portions at −10° C., the reaction mixture was stirred at −10° C. for 10 min. Then Br₂ (10.3 g, 64.7 mmol, 3.33 mL, 2.5 eq) was dropwise added at −10° C. and the reaction mixture was stirred at −10° C. for 1 h. The reaction mixture was poured into sat.aq.Na₂SO₃ (100 mL) and extracted with DCM (100 mL×3). The combined organic layers were washed with brine (100 mL×2), dried over Na₂SO₄, filtered and the filtrate was concentrated under reduced pressure to give the title compound (5.60 g, crude) as a yellow solid, which was used directly for next step without further purification.

### Preparation of 6-hydroxy-3-(4,4,5,5-tetramethyl-1,3,2-dioxaborolan-2-yl)-2-(trideut eriomethyl)benzaldehyde

To a solution of 3-bromo-6-hydroxy-2-(trideuteriomethyl)benzaldehyde (5.60 g, 25.7 mmol, 1 eq) in dioxane (100 mL) were added B₂Pin₂ (9.78 g, 38.5 mmol, 1.5 eq), Pd(dppf)Cl₂ (1.50 g, 2.05 mmol, 0.08 eq) and KOAc (6.30 g, 64.2 mmol, 2.5 eq) in portions at 20° C. The reaction mixture was degassed and purged with N₂ for 3 times, and then the mixture was directly moved to the oil-bath (pre-heated to 80° C.) and stirred at 80° C. for 4 h under N₂ atmosphere. After cooling to room temperature, the reaction mixture was filtered and the filtrate was concentrated under reduced pressure to give a residue. The residue was purified by flash silica gel chromatography (Biotage®; 120 g SepaFlash® Silica Flash Column, Eluent of 0~10% Ethyl acetate/Petroleum ether gradient @100 mL/min) to give the title compound (3.2 g, 12.07 mmol, 47.00% yield) as a yellow solid. ¹H NMR (CDCl₃, 400 MHz) δ 12.33 (s, 1H), 10.43 (s, 1H), 7.92 (d, J=8.8 Hz, 1H), 6.80 (d, J=8.4 Hz, 1H), 1.35 (s, 12H).

### Preparation of 3-[6-chloro-3-[1-(2-isopropyl-3,6-dimethyl-4-oxo-chromen-8-yl) ethylanzino]-2-pyridyl]-6-hydroxy-2-(trideuteriomethyl)benzaldehyde

The title compound was prepared as a yellow solid (3.00 g, 5.91 mmol, 83.87% yield) according to the similar procedure in Example 79 (utilizing the modification depicted in the Example 115 synthetic scheme), using 6-hydroxy-3-(4,4,5,5-tetramethyl-1,3,2-dioxaborolan-2-yl)-2-(trideuteriomethyl)benzaldehyde (2.80 g, 10.5 mmol, 1.5 eq) and 8-[l-[(2-bromo-6-chloro-3-pyridyl)amino]ethyl]-2-isopropyl-3,6-dimethyl-chromen-4-one (3.17 g, 7.04 mmol, 1 eq). ¹H NMR (DMSO-d₆, 400 MHz) δ 11.58 (s, 1H), 10.50 (d, J=12.0 Hz, 1H), 7.68 (s, 1H), 7.47-7.37 (m, 2H), 7.14 (d, J=8.0 Hz, 1H), 6.95 (d, J=8.4 Hz, 1H), 6.86-6.76 (m, 1H), 5.20-5.14 (m, 1H), 5.04-4.99 (m, 1H), 3.30-3.26 (m, 1H), 2.31 (s, 3H), 1.99 (s, 3H), 1.51-1.42 (m, 3H), 1.35-1.18 (m, 6H).

### Preparation of [4-[6-chloro-3-[1-(2-isopropyl-3,6-dimethyl-4-oxo-chromen-8-yl) ethylamino]-2-pyridyl]-2-formyl-3-(trideuteriomethyl)phenyl]trifluoromethanesulfonate

The title compound was prepared as a white solid (2.00 g, 3.12 mmol, 79.37% yield) according to the similar procedure in Example 79 (utilizing the modification depicted in the Example 115 synthetic scheme), using 3-[6-chloro-3-[1-(2-isopropyl-3,6-dimethyl-4-oxo-chromen-8-yl)ethylamino]-2-pyridyl]-6-hydroxy-2-(trideuteriomethyl)benzaldehyde (2.00 g, 3.94 mmol, 1 eq) and PhNTf₂ (2.11 g, 5.91 mmol, 1.5 eq). ¹H NMR (DMSO-d₆, 400 MHz) δ 10.50 (d, J=14.8 Hz, 1H), 7.75-7.68 (m, 2H), 7.53 (d, J=8.4 Hz, 1H), 7.47-7.39 (m, 1H), 7.26-7.19 (m, 1H), 6.89-6.80 (m, 1H), 5.53-5.47 (m, 1H), 5.08-5.00 (m, 1H), 3.32-3.26 (m, 1H), 2.32 (s, 3H), 2.00 (s, 3H), 1.47 (d, J=5.6 Hz, 3H), 1.33-1.24 (m, 6H).

### Preparation of 3-[6-chloro-3-[1-(2-isopropyl-3,6-dimethyl-4-oxo-chromen-8-yl) ethylamino]-2-pyridyl]-6-(4,4,5,5-tetramethyl-1,3,2-dioxaborolan-2-yl)-2-(trideuteriomethyl)benzaldehyde

The title compound was prepared as a gray solid (2.00 g, crude) according to the similar procedure in Example 79, using [4-[6-chloro-3-[1-(2-isopropyl-3,6-dimethyl-4-oxo-chromen-8-yl)ethylamino]-2-pyridyl]-2-formyl-3-(trideuteriomethyl)phenyl]trifluoromethanesulfonate (2.00 g, 3.12 mmol, 1 eq) and B₂Pin₂ (2.38 g, 9.37 mmol, 3 eq). The product was used directly for next step without further purification.

### Preparation of 8-[1-[[6-chloro-2-[1-hydroxy-5-(trideuteriomethyl)-2,3,1-benzoxa zaborinin-6-yl]-3-pyridyl]amino]ethyl]-2-isopropyl-3,6-dimethyl-chromen-4-one

The title compound was prepared as a white solid (400 mg, 750.69 μmol, 23.20% yield) according to the similar procedure in Example 79 (utilizing the modification depicted in the Example 115 synthetic scheme), using 3-[6-chloro-3-[1-(2-isopropyl-3,6-dimethyl-4-oxo-chromen-8-yl)ethylamino]-2-pyridyl]-6-(4,4,5,5-tetramethyl-1,3,2-dioxaborolan-2-yl)-2-(trideuteriomethyl)benzaldehyde (2.00 g, 3.24 mmol, 1 eq) and NH₂OH·HCl (450 mg, 6.47 mmol, 2 eq). ¹H NMR (DMSO-d₆, 400 MHz) δ 9.40 (s, 1H), 8.89-8.87 (m, 1H), 8.02 (d, J=7.6 Hz, 1H), 7.68-7.66 (m, 1H), 7.58 (t, J=6.8 Hz, 1H), 7.47-7.36 (m, 1H), 7.20 (t, J=7.6 Hz, 1H), 6.94-6.81 (m, 1H), 5.33-5.22 (m, 1H), 5.09-4.95 (m, 1H), 3.32-3.26 (m, 1H), 2.32 (s, 3H), 1.99 (s, 3H), 1.43 (t, J=6.0 Hz, 3H), 1.31-1.18 (m, 6H).

### Preparation of 8-[(1S)-1-[[6-chloro-2-[1-hydroxy-5-(trideuteriomethyl)-2,3,1-benzoxazaborinin-6-yl]-3-pyridyl]amino]ethyl]-2-isopropyl-3,6-dimethyl-chromen-4-one and 8-[(1R)-1-[[6-chloro-2-[1-hydroxy-5-(trideuteriomethyl)-2,3,1-benzoxazaborinin-6-yl]-3-pyridyl]amino]ethyl]-2-isopropyl-3,6-dimethyl-chromen-4-one

8-[1-[[6-chloro-2-[1-hydroxy-5-(trideuteriomethyl)-2,3,1-benzoxazaborinin-6-yl]-3-pyridyl]amino]ethyl]-2-isopropyl-3,6-dimethyl-chromen-4-one (500 mg, 938 μmol, 1 eq) was separated by SFC (column: DAICEL CHIRALPAK IG (250 mm×30 mm, 10 um); mobile phase: [CO₂—IPA]; B %: 65%, isocratic elution mode) to give the title compound, Isomer 1 (152.4 mg) as an off-white solid and the title compound, Isomer 2 (148.3 mg) as an off-white solid. Isomer 1: ¹H NMR (DMSO-d₆, 400 MHz) δ 9.40 (d, J=7.6 Hz, 1H), 8.89-8.87 (m, 1H), 8.02 (d, J=7.6 Hz, 1H), 7.69-7.56 (m, 2H), 7.47-7.36 (m, 1H), 7.22-7.18 (m, 1H), 6.95-6.82 (m, 1H), 5.31-5.20 (m, 1H), 5.08-4.95 (m, 1H), 3.30-3.26 (m, 1H), 2.32 (s, 3H), 1.99 (s, 3H), 1.49-1.39 (m, 3H), 1.31-1.18 (m, 6H). MS (ESI): mass calcd. For C₂₉H₂₆D₃BClN₃O₄ 532.21, m/z found 533.3 [M+H]⁺. HPLC: 99.04% (220 nm), 99.52% (254 nm). Chiral purity: 100% ee. Isomer 2: ¹H NMR (DMSO-d₆, 400 MHz) δ 9.40 (d, J=6.8 Hz, 1H), 8.89-8.87 (m, 1H), 8.02 (d, J=7.6 Hz, 1H), 7.69-7.56 (m, 2H), 7.47-7.36 (m, 1H), 7.22-7.18 (m, 1H), 6.95-6.82 (m, 1H), 5.31-5.20 (m, 1H), 5.08-4.95 (m, 1H), 3.30-3.26 (m, 1H), 2.32 (s, 3H), 1.99 (s, 3H), 1.49-1.39 (m, 3H), 1.31-1.18 (m, 6H). MS (ESI): mass calcd. For C₂₉H₂₆D₃BClN₃O₄ 532.21, m/z found 533.3 [M+H]⁺. HPLC: 99.75% (220 nm), 100.00% (254 nm). Chiral purity: 99.40% ee

### Example 116

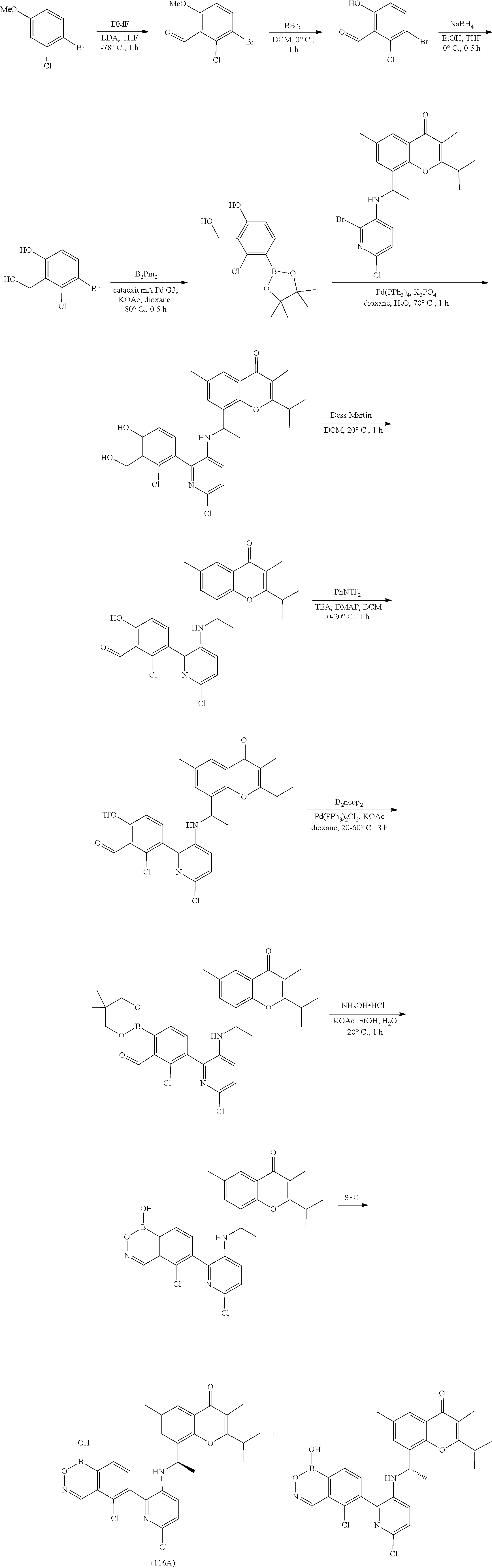

### Preparation of 3-bromo-2-chloro-6-methoxybenzaldehyde

To a solution of 1-bromo-2-chloro-4-methoxy-benzene (10.00 g, 45.15 mmol, 1 eq) in THF (100 mL) was added LDA (2 M, 27 mL, 1.2 eq) dropwise at −78° C., the reaction mixture was stirred at −78° C. for 20 min. Then DMF (4.29 g, 58.70 mmol, 4.52 mL, 1.3 eq) was added dropwise to the above mixture at −78° C. and the resulting reaction mixture was stirred at −78° C. for 40 min. The reaction mixture was quenched by addition sat.aq.NH₄Cl (50 mL) at 0° C. and extracted with EtOAc (30 mL×3). The combined organic layers were washed with brine (30 mL×2), dried over Na₂SO₄, filtered and concentrated under reduced pressure to give the title compound (10.00 g, 40.08 mmol, 88.77% yield) as yellow oil. ¹H NMR (CDCl₃ 400 MHz.) δ 10.43 (s, 1H), 7.74 (d, J=9.2 Hz, 1H), 6.85 (d, J=9.2 Hz, 1H), 3.92 (s, 3H).

### Preparation of 3-bromo-2-chloro-6-hydroxybenzaldehyde

To a solution of 3-bromo-2-chloro-6-methoxy-benzaldehyde (30.00 g, 120.25 mmol, 1 eq) in DCM (300 mL) was added BBr₃ (17.38 g, 69.37 mmol, 6.68 mL) dropwise at 0° C., the reaction mixture was stirred at 0° C. for 1 h. The reaction mixture was quenched by adding water (600 mL) and extracted with EtOAc (300 mL×3). The combined organic layers were washed with brine (200 mL×2), dried over Na₂SO₄, filtered and concentrated under reduced pressure to give a residue. The residue was purified by flash silica gel chromatography (Biotage®; 330 g SepaFlash® Silica Flash Column, Eluent of 15-21% Ethyl acetate/Petroleum ether gradient @150 mL/min) to give the title compound (24.00 g, crude) as a light yellow solid. ¹H NMR (CDCl₃ 400 MHz.) δ 12.03 (s, 1H), 10.44 (s, 1H), 7.70 (d, J=8.8 Hz, 1H), 6.85 (d, J=9.2 Hz, 1H).

### Preparation of 4-bromo-3-chloro-2-(hydroxymethyl)phenol

To a solution of 3-bromo-2-chloro-6-hydroxybenzaldehyde (2.70 g, 11.47 mmol, 1 eq) in THF (30 mL)/EtOH (5 mL) was added NaBH₄ (670 mg, 17.71 mmol, 1.54 eq) in portions at 0° C. The reaction mixture was stirred at 0° C. for 0.5 h. The reaction mixture was quenched by adding water (60 mL) and extracted with EtOAc (50 mL×3). The combined organic layers were washed with brine (50 mL), dried over anhydrous Na₂SO₄, filtered and concentrated under reduced pressure to give a residue.

The residue was purified by flash silica gel chromatography (Biotage®; 40 g SepaFlash® Silica Flash Column, Eluent of 30~40% Ethyl acetate/Petroleum ether gradient @80 mL/min) to give the title compound (1.20 g, 5.05 mmol, 44.07% yield) as a yellow solid. ¹H NMR (CDCl₃ 400 MHz.) δ 8.43 (s, 1H), 7.42 (d, J=8.8 Hz, 1H), 6.71 (d, J=8.8 Hz, 1H), 5.17 (s, 2H).

### Preparation of 3-chloro-2-(hydroxymethyl)-4-(4,4,5,5-tetramethyl-1,3,2-dioxaborolan-2-yl)phenol

A mixture of 4-bromo-3-chloro-2-(hydroxymethyl)phenol (1.00 g, 4.21 mmol, 1 eq), B₂Pin₂ (1.60 g, 6.32 mmol, 1.5 eq), KOAc (826 mg, 8.42 mmol, 2 eq) and catacxiumA Pd G3 (307 mg, 421.09 μmol, 0.1 eq) in dioxane (20 mL) was degassed and purged with N₂ for 3 times at 20° C., and then the mixture was heated to 80° C. and stirred at 80° C. for 0.5 h under N₂ atmosphere. The reaction mixture was filtered and the filtrate was concentrated under reduced pressure to give a residue. The residue was purified by prep-HPLC (column: Waters Xbridge Prep OBD C18 150×40 mm×10 um; mobile phase: [H₂O (10 mM NH₄HCO₃)-AC□]; gradient: 25%-55% B over 8.0 min) to the title compound (500 mg, crude) as a white solid, which was used directly for next step without further purification.

### Preparation of 8-(1-((6-chloro-2-(2-chloro-4-hydroxy-3-(hydroxymethyl)phenyl)pyridin-3-yl)amino)ethyl)-2-isopropyl-3,6-dimethyl-4H-chromen-4-one

The title compound was prepared as a yellow solid (0.50 g, crude) according to the procedures described herein and the synthetic scheme, using 8-(1-((2-bromo-6-chloropyridin-3-yl)amino)ethyl)-2-isopropyl-3,6-dimethyl-4H-chromen-4-one (1.00 g, 2.22 mmol, 1 eq) and 3-chloro-2-(hydroxymethyl)-4-(4,4,5,5-tetramethyl-1,3,2-dioxaborolan-2-yl)phenol (632 mg, 2.22 mmol, 1 eq). The product was used directly for next step without further purification.

### Preparation of 2-chloro-3-(6-chloro-3-((1-(2-isopropyl-3,6-dimethyl-4-oxo-4H-chromen-8-yl)ethyl)amino)pyridin-2-yl)-6-hydroxybenzaldehyde

To a solution of 8-(1-((6-chloro-2-(2-chloro-4-hydroxy-3-(hydroxymethyl)phenyl)pyridin-3-yl) amino)ethyl)-2-isopropyl-3,6-dimethyl-4H-chromen-4-one (1.20 g, 2.28 mmol, 1 eq) in DCM (20 mL) was added Dess-Martin (1.45 g, 3.41 mmol, 1.06 mL, 1.5 eq) in portions at 0° C. The reaction mixture was warmed to 20° C. and stirred at 20° C. for 1 h. The reaction mixture was quenched by adding water (60 mL) and extracted with DCM (30 mL×3). The combined organic layers were dried over anhydrous Na₂SO₄, filtered and concentrated under reduced pressure to give a residue. The crude product was purified by a silica gel column chromatography (PE:EtOAc=5:1-0:1) and further triturated with MTBE (20 mL) to give the title compound (0.88 g, 73% yield) as a yellow solid. ¹H NMR (CDCl₃ 400 MHz) δ 12.21 (s, 1H), 10.55 (d, J=4.0 Hz, 1H), 7.90 (s, 1H), 7.58-7.55 (m, 1H), 7.42-7.25 (m, 1H), 7.12-7.11 (m, 1H), 7.09-7.05 (m, 1H), 6.59-6.57 (m, 1H), 5.04-4.98 (m, 1H), 3.93-3.90 (m, 1H), 3.33-3.28 (m, 1H), 2.36 (d, J=6.8 Hz, 3H), 2.11 (s, 3H), 1.56-1.50 (m, 3H), 1.38-1.32 (m, 6H).

### Preparation of 3-chloro-4-(6-chloro-3-((1-(2-isopropyl-3,6-dimethyl-4-oxo-4H-chromen-8-yl)ethyl)amino)pyridin-2-yl)-2-formylphenyl trifluoromethanesulfonate

The title compound was prepared as a yellow solid (1.20 g, 1.83 mmol, 87.18% yield) according to the procedures described herein and the synthetic scheme, using 2-chloro-3-(6-chloro-3-((1-(2-isopropyl-3,6-dimethyl-4-oxo-4H-chromen-8-yl)ethyl)amino)pyridin-2-yl)-6-hydroxybenzaldehyde (1.10 g, 2.09 mmol, 1 eq) and PhNTf₂ (822 mg, 2.30 mmol, 1.1 eq). ¹H NMR (CDCl₃, 400 MHz) δ 10.56 (s, 1H), 7.91 (s, 1H), 7.79-7.75 (m, 1H), 7.49-7.47 (m, 1H), 7.42-7.40 (m, 1H), 7.13-7.08 (m, 1H), 6.66-6.64 (m, 1H), 5.07-5.00 (m, 1H), 3.84-3.83 (m, 1H), 3.34-3.29 (m, 1H), 2.37 (d, J=8.4 Hz, 3H), 2.12 (s, 3H), 1.58-1.53 (m, 3H), 1.38-1.31 (m, 6H).

### Preparation of 2-chloro-3-(6-chloro-3-((1-(2-isopropyl-3,6-dimethyl-4-oxo-4H-chromen-8-yl)ethyl)amino)pyridin-2-yl)-6-(5,5-dimethyl-1,3,2-dioxaborinan-2-yl)benzaldehyde

The title compound was prepared as a yellow solid (190 mg, crude) according to the procedures described herein and the synthetic scheme, using 3-chloro-4-(6-chloro-3-((1-(2-isopropyl-3,6-dimethyl-4-oxo-4H-chromen-8-yl)ethyl)amino)pyridin-2-yl)-2-formylphenyl trifluoromethanesulfonate (200 mg, 304.19 μmol, 1 eq) and B₂neop₂ (171 mg, 760 μmol, 2.5 eq). The product was used directly for next step.

### Preparation of 8-(1-((6-chloro-2-(5-chloro-1-hydroxy-1H-benzo[d][1,2,6]oxazaborinin-6-yl)pyridin-3-yl)amino)ethyl)-2-isopropyl-3,6-dimethyl-4H-chromen-4-one

The title compound was prepared as a white solid (150 mg, 271.90 μmol, 22.08% yield) according to the procedures described herein and the synthetic scheme, using 2-chloro-3-(6-chloro-3-((1-(2-isopropyl-3,6-dimethyl-4-oxo-4H-chromen-8-yl)ethyl)amino)pyridin-2-yl)-6-(5,5-dimethyl-1,3,2-dioxaborinan-2-yl)benzaldehyde (0.85 g, 1.23 mmol, 1 eq) and NH₂OH·HCl (171 mg, 2.46 mmol, 2 eq). ¹H NMR (DMSO-d₆, 400 MHz) δ 9.68-9.64 (m, 1H), 8.97-8.87 (m, 1H), 8.13-8.10 (m, 1H), 7.75-7.74 (m, 1H), 7.68-7.63 (m, 1H), 7.53-7.39 (m, 1H), 7.23-7.18 (m, 1H), 6.97-6.77 (m, 1H), 5.62-5.58 (m, 1H), 5.03-4.99 (m, 1H), 3.25-3.24 (m, 1H), 2.33-2.31 (m, 3H), 2.00 (s, 3H), 1.45-1.42 (m, 3H), 1.33-1.30 (m, 3H), 1.26-1.24 (m, 3H). MS (ESI): mass calcd. For C₂₈H₂₆BClN₃O₄ 549.14, m/z found 550.1 [M+H]⁺. HPLC: 99.74% (220 nm), 99.87% (254 nm)

### Preparation of (S)-8-(1-((6-chloro-2-(5-chloro-1-hydroxy-1H-benzo[d][1,2,6]oxaza borinin-6-yl)pyridin-3-yl)amino)ethyl)-2-isopropyl-3,6-dimethyl-4H-chromen-4-one and (R)-8-(1-((6-chloro-2-(5-chloro-1-hydroxy-1H-benzo[d][1,2,6]oxazaborinin-6-yl)pyridine-3-yl)amino)ethyl)-2-isopropyl-3,6-dimethyl-4H-chromen-4-one

8-(1-((6-chloro-2-(5-chloro-1-hydroxy-1H-benzo[d][1,2,6]oxazaborinin-6-yl)pyridin-3-yl)amino)ethyl)-2-isopropyl-3,6-dimethyl-4H-chromen-4-one (150 mg, 245 μmol, 1 eq) was separated by SFC (column: DAICEL CHIRALPAK IG (250 mm×30 mm×10 um); mobile phase: [CO₂—IPA]; B %:50%, isocratic elution mode) to give the title compound, Isomer 1 (48.6 mg, 87.51 μmol, 35.67% yield) as a white solid and the title compound, Isomer 2 (41.2 mg, 73.99 μmol, 30.16% yield) as a white solid. Isomer 1: ¹H NMR (DMSO-d₆, 400 MHz) δ 9.03-8.66 (m, 1H), 8.65-8.01 (m, 1H), 7.69-7.64 (m, 2H), 7.56-7.39 (m, 1H), 7.18-7.14 (m, 1H), 6.76-6.74 (m, 1H), 5.24-5.21 (m, 1H), 5.04-4.99 (m, 1H), 3.22-3.22 (m, 1H), 2.31 (s, 3H), 1.99 (s, 3H), 1.44-1.42 (m, 3H), 1.31-1.30 (m, 3H), 1.28-1.21 (m, 3H). MS (ESI): mass calcd. For C₂₈H₂₆BClN₃O₄ 549.14, m/z found 550.2 [M+H]⁺. HPLC: 99.08% (220 nm), 99.46% (254 nm). Chiral purity: 100% ee. Isomer 2: ¹H NMR (DMSO-d₆, 400 MHz) δ 9.66-9.60 (m, 1H), 8.97-8.63 (m, 1H), 8.13-8.11 (m, 1H), 7.75-7.67 (m, 2H), 7.63-7.54 (m, 1H), 7.39-7.16 (m, 1H), 6.79-6.76 (m, 1H), 5.60-5.58 (m, 1H), 5.04-4.99 (m, 1H), 3.23-3.23 (m, 1H), 2.33-2.31 (m, 3H), 1.99-1.97 (m, 3H), 1.45-1.42 (m, 3H), 1.31-1.21 (m, 6H). MS (ESI): mass calcd. For C₂₈H₂₆BClN₃O₄ 549.14, m/z found 550.2 [M+H]⁺. HPLC: 98.81% (220 nm), 99.17% (254 nm). Chiral purity: 100% ee

### Example 118

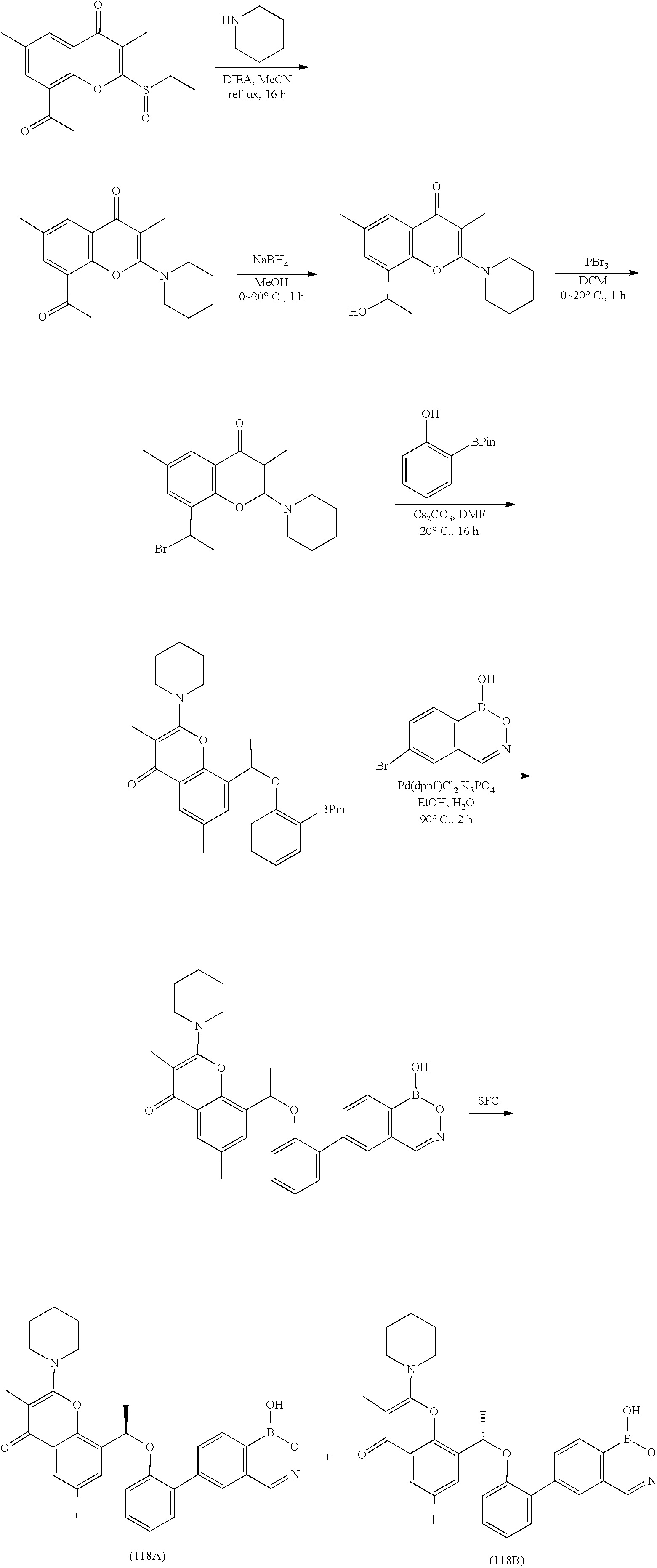

### Preparation of 8-acetyl-3,6-dimethyl-2-(1-piperidyl)chromen-4-one

To a solution of 8-acetyl-2-ethylsulfinyl-3,6-dimethyl-chromen-4-one (4.00 g, 13.68 mmol, 1 eq) and piperidine (1.75 g, 20.52 mmol, 2.03 mL, 1.5 eq) in MeCN (50 mL) was added DIEA (5.30 g, 41.05 mmol, 7.15 mL, 3 eq) dropwise at 20° C., the reaction mixture was stirred for 16 h under reflux. The reaction mixture was cooled to 20° C. and directly filtered. The filter cake was triturated with MeCN (20 mL) to give the title compound (3.30 g, 11.02 mmol, 80.57% yield) as a white solid. ¹H NMR (CDCl₃, 400 MHz) δ 8.17 (d, J=2.4 Hz, 1H), 7.78 (d, J=2.4 Hz, 1H), 3.42-3.41 (m, 4H), 2.73 (s, 3H), 2.46 (s, 3H), 2.05 (s, 3H), 1.73-1.71 (m, 6H).

### Preparation of 8-(1-hydroxyethyl)-3,6-dimethyl-2-(1-piperidyl)chromen-4-one

To s solution of 8-acetyl-3,6-dimethyl-2-(1-piperidyl)chromen-4-one (3.30 g, 11.0 mmol, 1 eq) in MeOH (50 mL) was added NaBH₄ (500 mg, 13.23 mmol, 1.2 eq) at 0° C. in portions, the reaction mixture was stirred at 20° C. for 1 h. The reaction mixture was poured into ice water (500 mL) at 0° C. and the resulting suspension was filtered. The filter cake was further triturated with H₂O (20 mL) at 20° C. for 10 min to give the title compound (2.00 g, 6.64 mmol, 60.20% yield) as a white solid. ¹H NMR (CDCl₃, 400 MHz) δ 7.71 (d, J=2.0 Hz, 1H), 7.81 (d, J=2.0 Hz, 1H), 5.35-5.31 (m, 1H), 3.37-3.35 (m, 4H), 2.36 (s, 3H), 2.02 (s, 3H), 1.72-1.71 (m, 6H), 1.54 (d, J=6.4 Hz, 3H).

### Preparation of 8-(1-bromoethyl)-3,6-dimethyl-2-(1-piperidyl)chromen-4-one

To s solution of 8-(1-hydroxyethyl)-3,6-dimethyl-2-(1-piperidyl)chromen-4-one (2.00 g, 6.64 mmol, 1 eq) in DCM (20 mL) was added PBr₃ (2.16 g, 7.96 mmol, 1.2 eq) at 0° C. in portions, then the reaction mixture was stirred at 20° C. for 1 h. The reaction mixture was poured into ice-water (w/w=1/1) (100 mL) and extracted with DCM (50 mL×3). The combined organic phase was washed with brine (20 mL), dried with anhydrous Na₂SO₄, filtered and concentrated in vacuum. The residue was purified by flash silica gel chromatography (ISCO®; 40 g SepaFlash® Silica Flash Column, Eluent of 0~50% Ethyl acetate/Petroleum ether gradient @50 mL/min) to give the title compound (1.00 g, 2.75 mmol, 41.37% yield) as a white solid. ¹H NMR (CDCl₃, 400 MHz) δ 7.94 (d, J=1.2 Hz, 1H), 7.81 (d, J=2.0 Hz, 1H), 5.70-5.65 (m, 1H), 3.47-3.44 (m, 4H), 2.44 (s, 3H), 2.15 (d, J=7.2 Hz, 3H), 2.05 (s, 3H), 1.77-1.74 (m, 6H).

### Preparation of 3,6-dimethyl-2-(1-piperidyl)-8-[1-[2-(4,4,5,5-tetramethyl-1,3,2-dioxabo rolan-2-yl)phenoxy]ethyl]chromen-4-one

A mixture of 8-(1-bromoethyl)-3,6-dimethyl-2-(1-piperidyl)chromen-4-one (350 mg, 961 mol, 1 eq), 2-(4,4,5,5-tetramethyl-1,3,2-dioxaborolan-2-yl)phenol (275 mg, 1.25 mmol, 1.3 eq) and Cs₂CO₃ (500 mg, 1.54 mmol, 1.6 eq) in DMF (7 mL) was stirred at 20° C. for 16 h. The reaction mixture was poured into ice-water (w/w=1/1) (50 mL) and extracted with EtOAc (30 mL×3). The combined organic phase was washed with brine (20 mL×2), dried with anhydrous Na₂SO₄, filtered and concentrated in vacuum to give the title compound (500 mg, crude) as a yellow solid, which was used directly for next step without further purification. ¹H NMR (DMSO-d₆, 400 MHz) δ 7.88 (d, J=2.0 Hz, 1H), 7.81 (d, J=2.0 Hz, 1H), 6.66 (dd, J=1.6 Hz, 7.2 Hz, 1H), 7.30-7.29 (m, 1H), 6.96-6.94 (m, 1H), 6.74-6.72 (m, 1H), 5.86-5.81 (m, 1H), 3.35-3.34 (m, 4H), 2.42 (s, 3H), 2.05 (s, 3H), 2.05 (s, 3H), 1.71-1.67 (m, 6H), 1.37 (s, 12H).

### Preparation of 8-[1-[2-(1-hydroxy-2,3,1-benzoxazaborinin-6-yl)phenoxy]ethyl]-3,6-dimethyl-2-(1-piperidyl)chromen-4-one

A mixture of 3,6-dimethyl-2-(1-piperidyl)-8-[1-[2-(4,4,5,5-tetramethyl-1,3,2-dioxaborolan-2-yl)phenoxy]ethyl]chromen-4-one (450 mg, 894 μmol, 1 eq), 6-bromo-1-hydroxy-2,3,1-benzoxazaborinine (303 mg, 1.34 mmol, 1.5 eq), Pd(dppf)Cl₂ (65.4 mg, 89.4 μmol, 0.1 eq) and K₃PO₄ (380 mg, 1.79 mmol, 2 eq) in EtOH (8 mL)/H₂O (1 mL) was de-gassed and then heated to 90° C. for 2 h under N₂. The reaction mixture was filtered and the filtrate was concentrated in vacuo. The residue was purified by prep-HPLC (column: Waters Xbridge Prep OBD C18 150×40 mm×10 um; mobile phase: [H₂O (10 mM NH₄HCO₃)-AC□]; gradient: 45%-75% B over 8.0 min) to give the title compound (65.2 mg, 125 gmol) as a yellow solid. ¹H NMR (DMSO-d₆, 400 MHz) δ 9.38 (s, 1H), 8.60 (s, 1H), 8.08-8.06 (m, 1H), 7.88-7.85 (m, 2H), 7.63 (s, 1H), 7.39 (d, J=7.6 Hz, 1H), 7.34-7.33 (m, 2H), 7.10-7.07 (m, 2H), 5.84-5.80 (m, 1H), 3.21-3.20 (m, 4H), 2.26 (s, 3H), 1.89-1.85 (m, 3H), 1.61 (d, J=6.4 Hz, 3H), 1.54 (s, 6H). MS (ESI): mass calcd. For C₃₁H₃₁BN₂O₅ 522.23, m/z found 523.1 [M+H]⁺. HPLC: 97.23% (220 nm), 95.30% (254 nm).

### Preparation of (S)-8-(1-(2-(1-hydroxy-1H-benzo[d][1,2,6]oxazahorinin-6-yl)phenoxy) ethyl)-3,6-dimethyl-2-(piperidin-1-yl)-4H-chromen-4-one and (R)-8-(1-(2-(1-hydroxy-1H-benzo[d][1,2,6]oxazaborinin-6-yl)phenoxy)ethyl)-3,6-dimethyl-2-(piperidin-1-yl)-4H-chromen-4-one

8-(1-(2-(1-hydroxy-1H-benzo[d][1,2,6]oxazaborinin-6-yl)phenoxy)ethyl)-3,6-dimethyl-2-(piperidin-1-yl)-4H-chromen-4-one (60 mg, 114 μmol, 1 eq) was separated by SFC (column: ChiralPak IH, 250×30 mm×10 um; mobile phase: [CO₂-EtOH (0.1% □H3H₂O)]; B %:40%, isocratic elution mode) to give the title compound, Isomer 1 (25.4 mg, 48.62 μmol, 42.33% yield) as a white solid and the title compound, Isomer 2 (25.7 mg, 49.20 μmol, 42.83% yield) as a white solid. Isomer 1: ¹H NMR (DMSO-d₆, 400 MHz) δ 9.42-9.39 (m, 1H), 8.59-8.58 (m, 1H), 8.08-8.06 (m, 1H), 7.85 (s, 2H), 7.64 (s, 1H), 7.39-7.31 (m, 3H), 7.09-6.97 (m, 2H), 5.83-5.82 (m, 1H), 3.21-3.15 (m, 4H), 2.26 (s, 3H), 1.88-1.85 (m, 3H), 1.61 (d, J=6.4 Hz, 3H), 1.59-1.54 (m, 6H). MS (ESI): mass calcd. For C₃₁H₃₁BN₂O₅ 522.23, m/z found 523.2 [M+H]⁺. HPLC: 98.51% (220 nm), 99.18% (254 nm). Chiral purity: 99.20% ee. Isomer 2: ¹H NMR (DMSO-d₆, 400 MHz) δ 9.41 (s, 1H), 8.60 (s, 1H), 8.08-8.07 (m, 1H), 7.85 (s, 2H), 7.63 (s, 1H), 7.40-7.38 (m, 1H), 7.34-7.34 (m, 2H), 7.10-7.07 (m, 2H), 5.84-5.80 (m, 1H), 3.24-3.17 (m, 4H), 2.26 (s, 3H), 1.89-1.85 (m, 3H), 1.61 (d, J=6.0 Hz, 3H), 1.54 (s, 6H). MS (ESI): mass calcd. For C₃₁H₃₁BN₂O₅ 522.23, m/z found 523.2 [M+H]⁺. HPLC: 97.91% (220 nm), 98.77% (254 nm). Chiral purity: 97.84% ee.

### Example 119

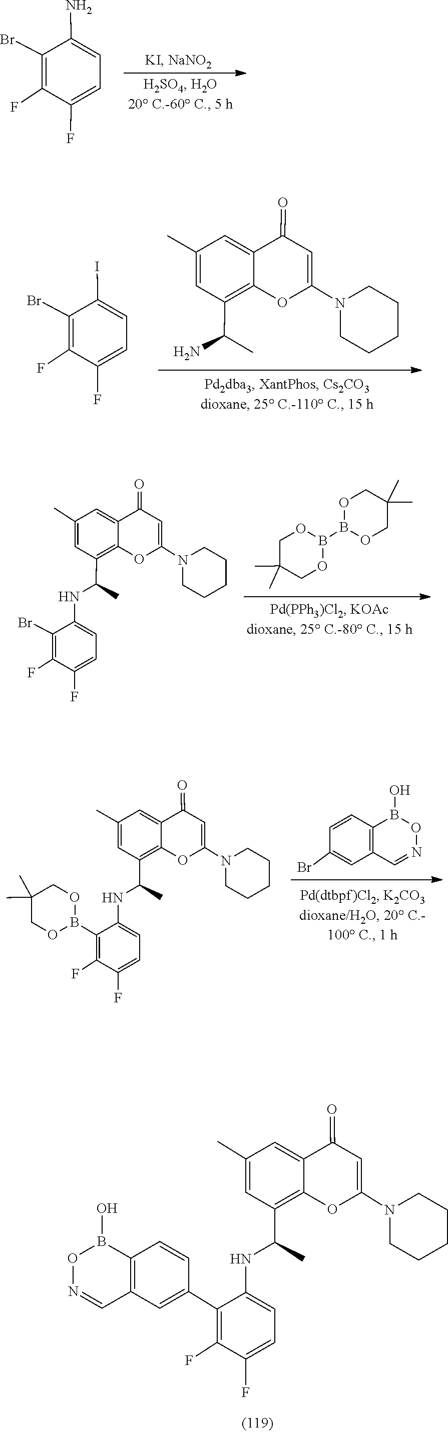

### Preparation of 3-bromo-1,2-difluoro-4-iodo-benzene

To a solution of 2-bromo-3,4-difluoro-aniline (1.0 g, 4.81 mmol, 1 eq) in H₂O (6 mL)/H₂SO₄ (73.5 mmol, 4 mL, 15.30 eq) was added NaNO₂ (431 mg, 6.25 mmol, 1.3 eq) in portions at 0° C. and stirred for 30 min. Then to the above mixture was added a solution of KI (957 mg, 5.77 mmol, 1.2 eq) in H₂O (2 mL) at 0° C. The resulting mixture was heated to 60° C. and stirred for 4.5 h. The reaction mixture was cooled to 20° C., poured into H₂O (10 mL) and extracted with DCM (20 mL×3). The combined organic layers were washed with sat.aq NaHCO₃ (30 mL×2), then sat.aq Na₂S₂O₃ (30 mL×2), dried over Na₂SO₄, filtered and concentrated under reduced pressure to give a residue. The residue was purified by flash silica gel chromatography (Biotage®; 20 g SepaFlash® Silica Flash Column, Eluent of 0~5% Ethyl acetate/Petroleum ether gradient @50 mL/min) to give the title compound (0.9 g, 2.82 mmol, 58.71% yield) as a yellow solid. ¹H NMR (DMSO-d₆, 400 MHz) δ 7.87-7.80 (m, 1H), 7.40-7.30 (m, 1H).

### Preparation of (R)-8-(1-((2-bromo-3,4-difluorophenyl)amino)ethyl)-6-methyl-2-(piperidin-1-yl)-4H-chromen-4-one

The title compound was prepared as a yellow solid (450 mg, 942.72 μmol, 67.49% yield) according to the similar procedure in Example 78, using 8-[(1R)-1-aminoethyl]-6-methyl-2-(1-piperidyl)chromen-4-one (400 mg, 1.40 mmol, 1 eq) and 3-bromo-1,2-difluoro-4-iodo-benzene (891 mg, 2.79 mmol, 2 eq). H NMR (DMSO-d₆, 400 MHz) δ 7.59 (s, 1H), 7.40 (d, J=1.6 Hz, 1H), 7.17 (q, J=9.6 Hz, 1H), 6.22-6.14 (m, 1H), 5.52 (d, J=7.2 Hz, 1H), 5.51 (s, 1H), 5.09-4.95 (m, 1H), 3.53 (s, 4H), 2.30 (s, 3H), 1.65-1.54 (m, 9H).

### Preparation of (R)-8-(1-((2-(5,5-dimethyl-1,3,2-dioxaborinan-2-yl)-3,4-difluorophenyl)amino)ethyl)-6-methyl-2-(piperidin-1-yl)-4H-chromen-4-one

The title compound was prepared as a yellow solid (90 mg, 70.54 μmol, 16.83% yield) according to the procedures described herein and the synthetic scheme, using 8-[(1R)-1-(2-bromo-3,4-difluoro-anilino)ethyl]-6-methyl-2-(1-piperidyl) chromen-4-one (400 mg, 838 μmol, 1 eq) and 2-(5,5-dimethyl-1,3,2-dioxaborinan-2-yl)-5,5-dimethyl-1,3,2-dioxaborinane (568 mg, 2.52 mmol, 3 eq).

### Preparation of 8-[(1R)-1-[3,4-difluoro-2-(1-hydroxy-2,3,1-benzoxazaborinin-6-yl) anilino]ethyl]-6-methyl-2-(1-piperidyl)chromen-4-one

The title compound was prepared as an off white solid (20.6 mg, 36.98 μmol, 29.49% yield) according to the procedures described herein and the synthetic scheme, using 8-[(1R)-1-[2-(5,5-dimethyl-1,3,2-dioxaborinan-2-yl)-3,4-difluoro-anilino]ethyl]-6-methyl-2-(1-piperidyl)chromen-4-one (80 mg, 125.4 μmol, 1 eq) and 6-bromo-1-hydroxy-2,3,1-benzox azaborinine (34 mg, 150.5 μmol, 1.2 eq). ¹H NMR (DMSO-d₆, 400 MHz) δ 9.49 (s, 1H), 8.65 (s, 1H), 8.17 (d, J=7.2 Hz, 1H), 7.84-7.63 (m, 2H), 7.56 (s, 1H), 7.33 (s, 1H), 7.23-7.14 (m, 1H), 6.34-6.24 (m, 1H), 5.46 (s, 1H), 5.04-4.91 (m, 1H), 4.87-4.70 (m, 1H), 3.42 (s, 4H), 2.31 (s, 3H), 1.62-1.52 (m, 6H), 1.37 (d, J=6.4 Hz, 3H). MS (ESI): mass calcd. For C₃₀H₂₈BF₂N₃O₄ 543.38, m/z found 544.3 [M+H]⁺. HPLC: 97.54 (220 nm), 98.39 (254 nm). Chiral purity: 98.98% ee.

### Example 121

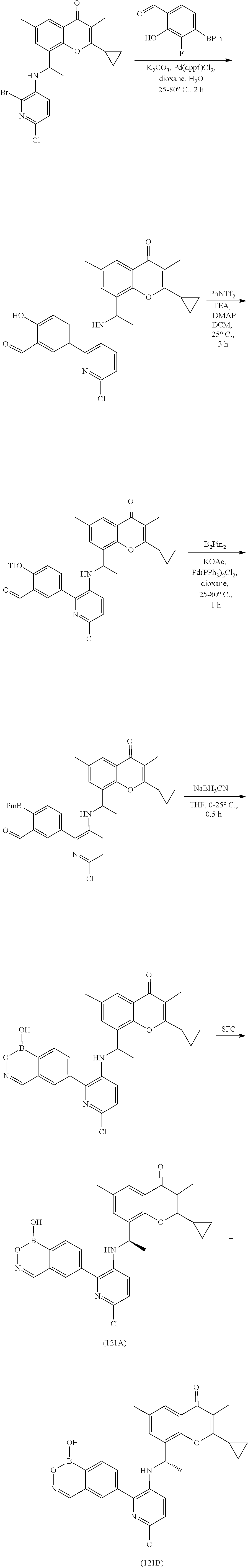

### Preparation of 4-(6-chloro-3-((1-(2-cyclopropyl-3,6-dimethyl-4-oxo-4H-chromen-8-yl) ethyl)amino)pyridin-2-yl)-3-fluoro-2-hydroxybenzaldehyde

The title compound was prepared as yellow oil (5.9 g, crude) according to the procedures described herein and the synthetic scheme, using 8-[1-[(2-bromo-6-chloro-3-pyridyl)amino]ethyl]-2-cyclopropyl-3,6-dimethyl-chromen-4-one (4.00 g, 8.93 mmol, 1 eq) and 3-fluoro-2-hydroxy-4-(4,4,5,5-tetramethyl-1,3,2-dioxaborolan-2-yl)benzaldehyde (4.75 g, 17.87 mmol, 2 eq). ¹H NMR (DMSO-d₆, 400 MHz) δ 11.04 (s, 1H), 10.35 (s, 1H), 7.66 (s, 1H), 7.57 (d, J=8.0 Hz, 1H), 7.42 (d, J=2.0 Hz, 1H), 7.19 (d, J=8.4 Hz, 1H), 7.03 (t, J=6.0 Hz, 1H), 6.82 (d, J=8.8 Hz, 1H), 5.70 (d, J=6.8 Hz, 1H), 4.86 (t, J=6.8 Hz, 1H), 2.31 (s, 3H), 2.09 (s, 3H), 1.44-1.41 (m 3H), 1.19-1.15 (m, 5H).

### Preparation of 3-(6-chloro-3-((1-(2-cyclopropyl-3,6-dimethyl-4-oxo-4H-chromen-8-yl) ethyl)amino)pyridin-2-yl)-2-fluoro-6-formylphenyl trifluoromethanesulfonate

The title compound was prepared as a yellow solid (3.10 g, 4.85 mmol, 45.54% yield) according to the procedures described herein and the synthetic scheme, using 4-[6-chloro-3-[1-(2-cyclopropyl-3,6-dimethyl-4-oxo-chromen-8-yl)ethylamino]-2-pyridyl]-3-fluoro-2-hydroxy-benzaldehyde (5.40 g, 10.65 mmol, 1 eq) and PhNTf₂ (5.71 g, 15.98 mmol, 1.5 eq). ¹H NMR (DMSO-d₆, 400 MHz) δ 10.16 (s, 1H), 8.08 (d, J=8.4 Hz, 1H), 7.94 (t, J=8.0 Hz, 1H), 7.67 (s, 1H), 7.43 (s, 1H), 7.26 (d, J=8.8 Hz, 1H), 6.86 (d, J=9.2 Hz, 1H), 6.06 (d, J=6.4 Hz, 1H), 4.89 (t, J=6.4 Hz, 1H), 2.31 (s, 3H), 2.10 (s, 3H), 1.41 (d, J=6.4 Hz, 3H), 1.25-1.23 (m, 1H), 1.19-1.15 (m, 4H).

### Preparation of 4-(6-chloro-3-((1-(2-cyclopropyl-3,6-dimethyl-4-oxo-4H-chromen-8-yl) ethyl)amino)pyridin-2-yl)-3-fluoro-2-(4,4,5,5-tetramethyl-1,3,2-dioxaborolan-2-yl)benzaldehyde

The title compound was prepared as an off white solid (1.68 g, crude) according to the procedures described herein and the synthetic scheme, using [3-[6-chloro-3-[1-(2-cyclopropyl-3,6-dimethyl-4-oxo-chromen-8-yl)ethylamino]-2-pyridyl]-2-fluoro-6-formyl-phenyl]trifluoromethanesulfonate (1.70 g, 2.66 mmol, 1 eq) and B₂Pin₂ (1.35 g, 5.32 mmol, 2 eq). ¹H NMR (DMSO-d₆, 400 MHz) δ 10.07 (s, 1H), 7.95 (d, J=8.0 Hz, 1H), 7.79 (t, J=8.0 Hz, 1H), 7.66 (s, 1H), 7.43 (s, 1H), 7.20 (d, J=8.8 Hz, 1H), 6.84 (d, J=8.8 Hz, 1H), 5.69 (d, J=6.8 Hz, 1H), 4.87 (t, J=6.8 Hz, 1H), 2.31 (s, 3H), 2.09 (s, 3H), 1.40 (d, J=6.8 Hz, 3H), 1.35 (s, 12H), 1.10-1.06 (m, 5H).

### Preparation of 8-(1-((6-chloro-2-(7-fluoro-1-hydroxy-1,3-dihydrobenzo[c][1,2]oxaborol-6-yl)pyridin-3-yl)amino)ethyl)-2-cyclopropyl-3,6-dimethyl-4H-chromen-4-one

To a solution of 4-[6-chloro-3-[1-(2-cyclopropyl-3,6-dimethyl-4-oxo-chromen-8-yl)ethylamino]-2-pyridyl]-3-fluoro-2-(4,4,5,5-tetramethyl-1,3,2-dioxaborolan-2-yl)benzaldehyde (1.68 g, 2.72 mmol, 1 eq) in THF (18 mL) was added NaBH₃CN (256 mg, 4.08 mmol, 1.5 eq) in portions slowly at 0° C. The reaction mixture was warmed up to 25° C. and stirred at 25° C. for 0.5 h. The reaction mixture was quenched by H₂O (15 mL), and then extracted with EtOAc (15 mL×3). The combined organic layers were washed with brine (10 mL×2), dried over Na₂SO₄, filtered and concentrated under reduced pressure to give a residue. The residue was purified by prep-HPLC (column: Welch Xtimate C18 180×70 mm×10 um; mobile phase: [H₂O (10 mM NH₄HCO₃)-AC□]; gradient:32%-62% B over 17.0 min) to give the title compound (344 mg, 641.22 μmol, 22.37% yield) as a white solid. ¹H NMR (DMSO-d₆, 400 MHz) δ 9.33 (s, 1H), 7.65 (s, 1H), 7.53 (t, J=7.2 Hz, 1H), 7.42 (s, 1H), 7.33 (d, J=8.0 Hz, 1H), 7.17 (d, J=8.8 Hz, 1H), 6.83 (d, J=8.8 Hz, 1H), 5.38 (d, J=6.8 Hz, 1H), 5.06 (s, 2H), 4.83 (t, J=6.8 Hz, 1H), 2.31 (s, 3H), 2.09 (s, 3H), 1.40 (d, J=7.6 Hz, 3H), 1.19-1.17 (m, 1H), 1.08-1.02 (m, 2H), 0.86-0.83 (m, 2H).

### Preparation of (S)-8-(1-((6-chloro-2-(7-fluoro-1-hydroxy-1,3-dihydrobenzo[c][1,2]oxaborol-6-yl)pyridin-3-yl)amino)ethyl)-2-cyclopropyl-3,6-dimethyl-4H-chromen-4-one and (R)-8-(1-((6-chloro-2-(7-fluoro-1-hydroxy-1,3-dihydrobenzo[c][1,2]oxaborol-6-yl)pyridin-3-yl)amino)ethyl)-2-cyclopropyl-3,6-dimethyl-4H-chromen-4-one

8-[1-[[6-chloro-2-(7-fluoro-1-hydroxy-3H-2,1-benzoxaborol-6-yl)-3-pyridyl]amino]ethyl]-2-cyclopropyl-3,6-dimethyl-chromen-4-one (344 mg, 641.22 μmol, 1 eq) was separated by SFC (column: DAICEL CHIRALPAK AD (250 mm×30 mm, 10 um); mobile phase: [CO2-MeOH]; B %: 35%, isocratic elution mode) to give the title compound, Isomer 1 (108.5 mg, 202.66 μmol, 30.56% yield) as a white solid and the title compound, Isomer 2 (100.8 mg, 187.50 μmol, 28.28% yield) as a white solid. Isomer 1: ¹H NMR (DMSO-d₆, 400 MHz) δ 9.33 (s, 1H), 7.65 (s, 1H), 7.53 (t, J=7.2 Hz, 1H), 7.42 (s, 1H), 7.33 (d, J=8.0 Hz, 1H), 7.17 (d, J=8.8 Hz, 1H), 6.83 (d, J=8.8 Hz, 1H), 5.38 (d, J=6.8 Hz, 1H), 5.06 (s, 2H), 4.82 (t, J=7.2 Hz, 1H), 2.31 (s, 3H), 2.29-2.27 (m, 1H), 2.09 (s, 3H), 1.40 (d, J=6.8 Hz, 3H), 1.10-1.05 (m, 4H). MS (ESI): mass calcd. For C₂₈H₂₅BClFN₂O₄ 518.16, m/z found 519.2 [M+H]⁺. HPLC: 96.90% (220 nm), 97.54% (254 nm). Chiral purity: 100.00% ee. Isomer 2: ¹H NMR (DMSO-d₆, 400 MHz) δ 9.33 (s, 1H), 7.65 (s, 1H), 7.53 (t, J=7.2 Hz, 1H), 7.42 (s, 1H), 7.33 (d, J=8.0 Hz, 1H), 7.17 (d, J=8.8 Hz, 1H), 6.83 (d, J=8.8 Hz, 1H), 5.38 (d, J=6.8 Hz, 1H), 5.06 (s, 2H), 4.82 (t, J=7.2 Hz, 1H), 2.31 (s, 3H), 2.29-2.27 (m, 1H), 2.09 (s, 3H), 1.40 (d, J=6.8 Hz, 3H), 1.10-1.05 (m, 4H). MS (ESI): mass calcd. For C₂₈H₂₅BClFN₂O₄ 518.16, m/z found 519.2 [M+H]⁺. HPLC: 96.57% (220 nm), 97.22% (254 nm). Chiral purity: 99.56% ee

### BIOLOGICAL EXAMPLES

The compounds of the present disclosure may be tested in multiple assays.

pAKT

Compounds were tested in cancer cell lines with or without PI3Kα mutations including SKBR3 (wt), MCF7 (E545K), and T-47D (H1047R) cancer cell lines purchased from ATCC. pAKT1/2/3 (Ser473) HTRF kits were purchased from Revvity. In brief, the assay utilizes a plate-based assay format to detect endogenous AKT rapidly and directly in cells when Ser473 is phosphorylated. Adherent cells are lysed, then subjected to an Eu³⁺-Cryptate (donor) and d2 (acceptor) antibodies. Emission from the donor antibody triggers a Fluorescence Resonance Energy Transfer (FRET) at the acceptor antibody. The signal directly corresponds to pAKT at Ser473.

Cells were treated with compounds in dose response titration for 6 hours at 37° C. with 5% CO₂ in 96-well tissue culture treated plates. Cells were assayed in media containing 10% FBS and penicillin-streptomyocin. After compound treatment, cells were lysed with 20-25 μL of 1× supplemented lysis buffer for 30 minutes. During cellular lysis, antibodies were prepared according to manufacturer's instructions. Briefly, antibodies were diluted in detection buffer and pre-mixed at a 1:1 volume of pAKT Eu antibody and pAKT d2 antibody. Ten L of lysate was transferred to a 384-well plate where the lysates were incubated at room temperature with 2 L of pre-mixed pAKT antibodies overnight. Plates were read on a Cytation 5 plate reader (BioTek) where 620 and 665 nm wavelength emissions were measured. The ratio between 665 nm and 620 nm was used to generate the ICsₒ for tested compounds, where ratios were normalized to DMSO control and background signal.

### ADP-Glo

Compound activity was determined biochemically via the ADP-Glo Kinase Assay purchased from Promega in conjunction with PIP₂:PS lipid kinase substrate from ThermoFisher Scientific and (p110a/p85a) and (p110a [H1047R]/p85a) enzymes from Viva. The assay was performed according to the manufacturer's instructions with compounds pre-incubated with 1 nM of enzyme and 2.5 nM of pY2-peptide derived from PDGFRb for 4 hours. The reaction proceeded by addition of 1 mM ATP and either 200 mM, 50 mM, or 25 mM of lipid substrate. Ideal enzyme and substrate concentrations were determined empirically through enzyme titration to represent 5% product formation as recommended by the manufacturer. The kinase reaction was allowed to proceed for one hour prior to measurement of bioluminescent light output on a Cytation 5 plate reader with a 0.5 second integration time. Compounds were tested in seven point dose response and IC₅₀ were determined by normalization to DMSO and no enzyme controls.

Tested compounds with activity in the tested concentration range are reported: <500 nM ++++, 501 nM to 1 μM+++, 1 μM to 10 μM++, and measured as either above 5 μM or above 10 μM+.

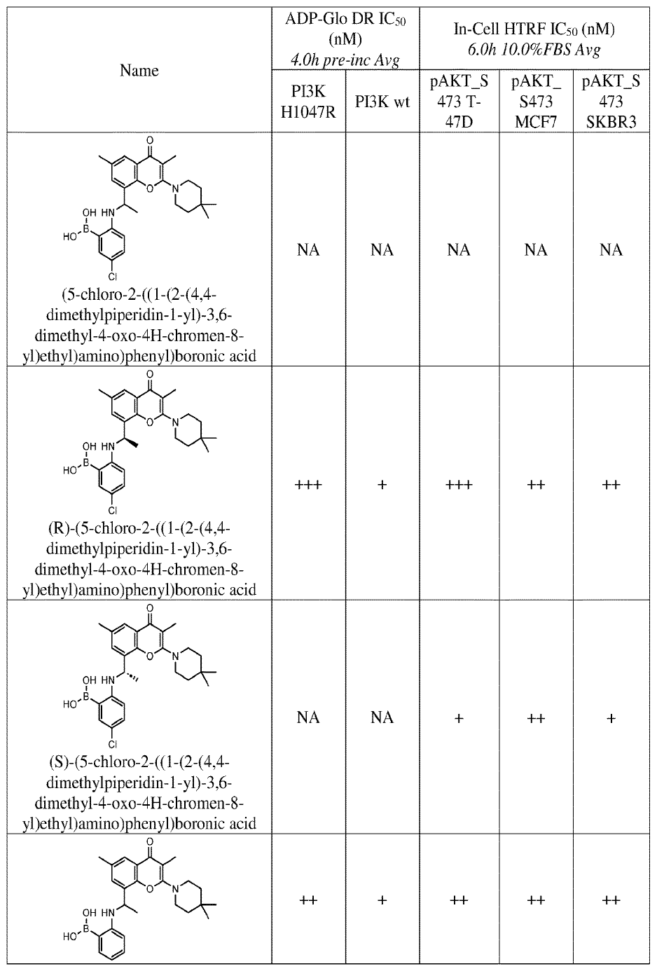

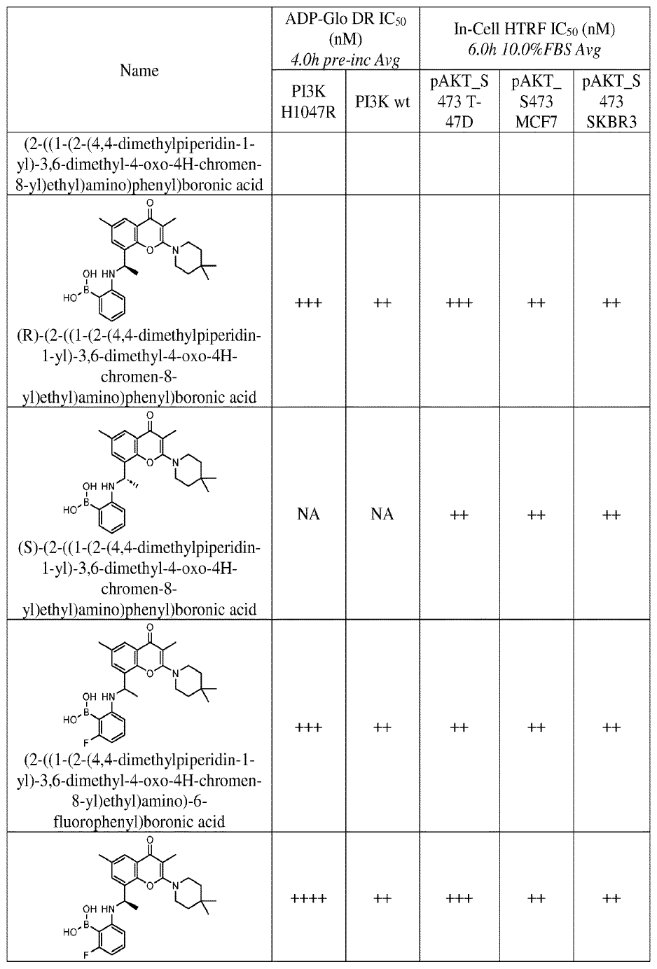

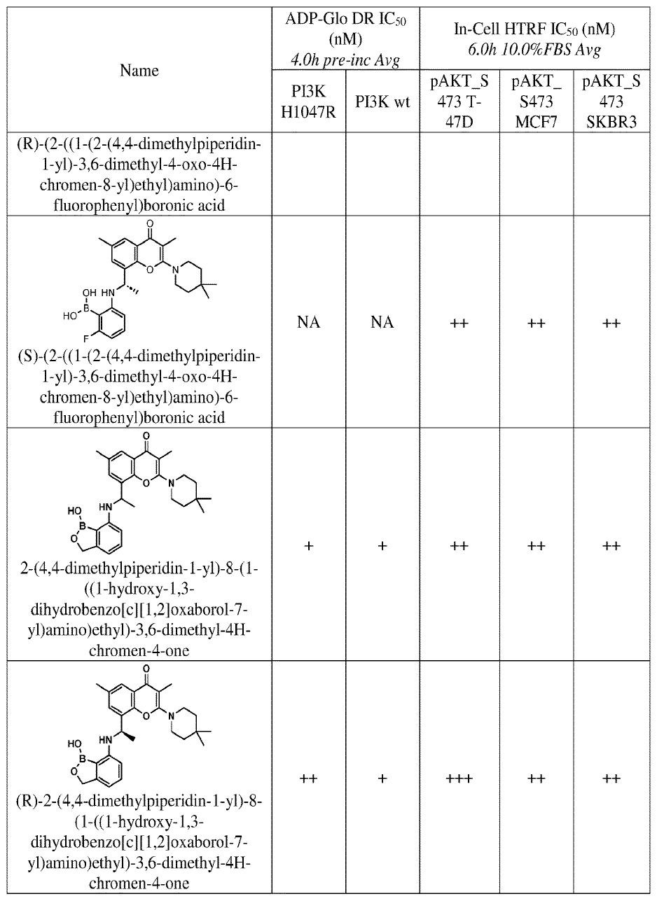

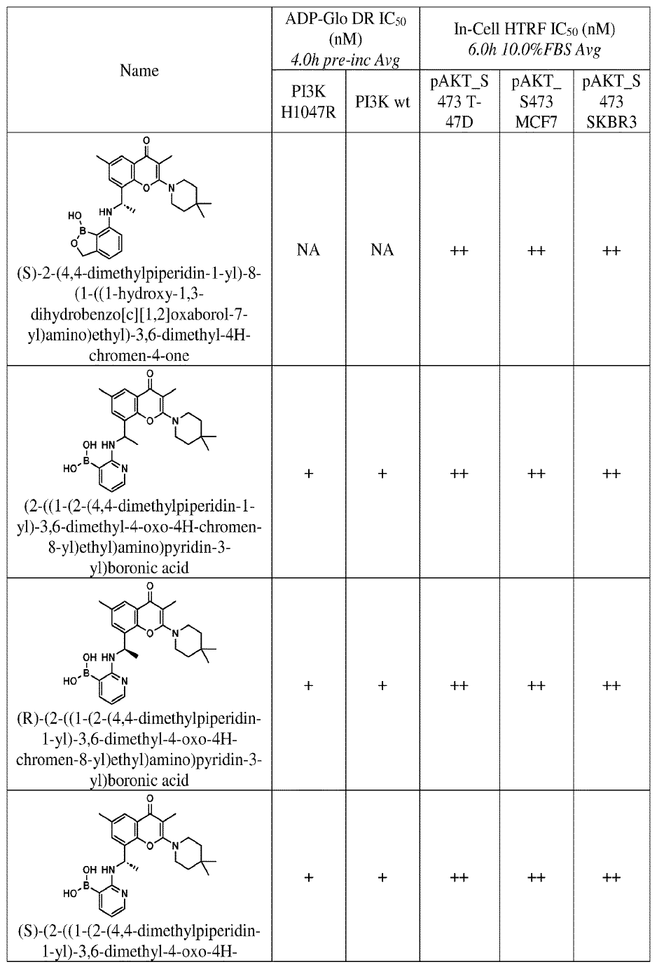

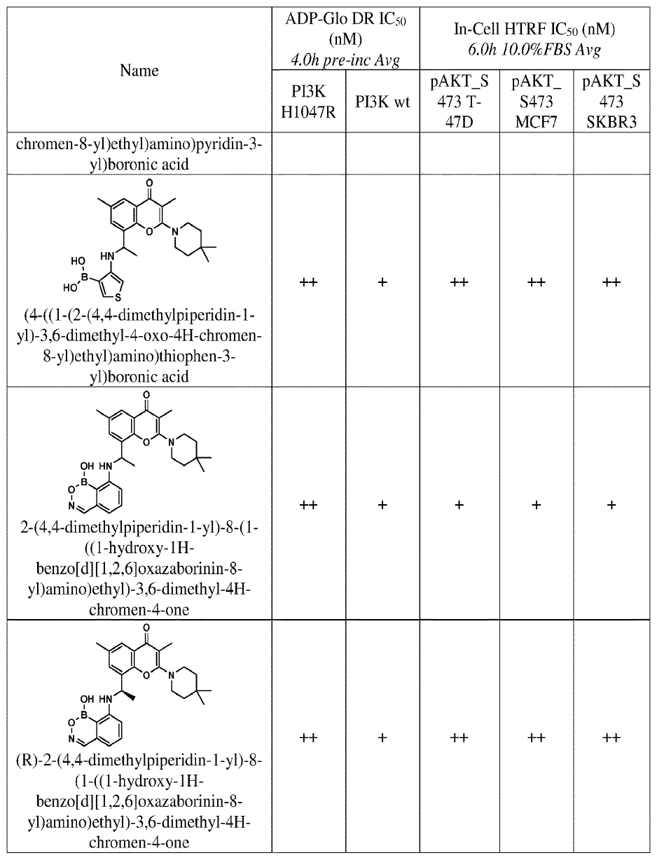

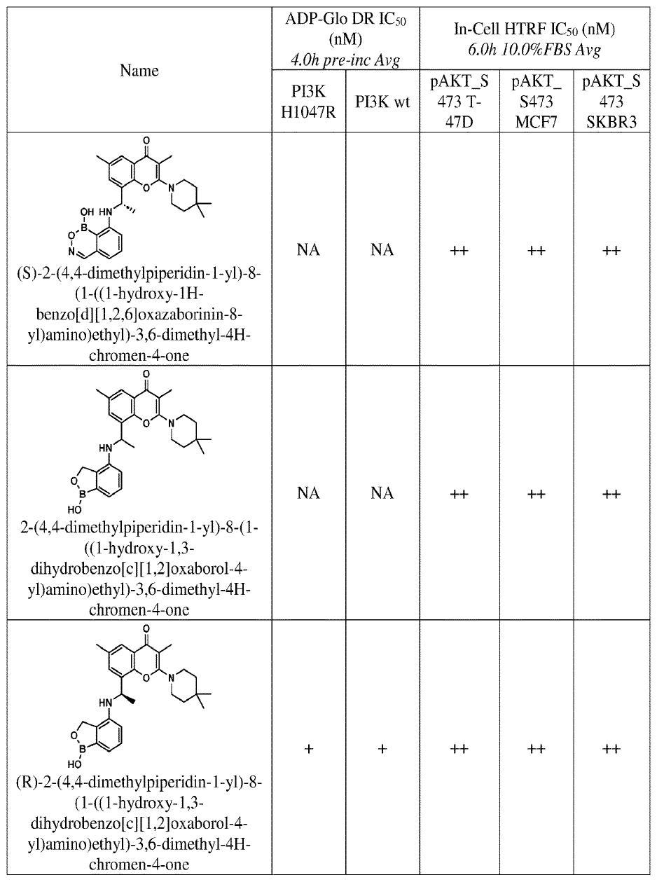

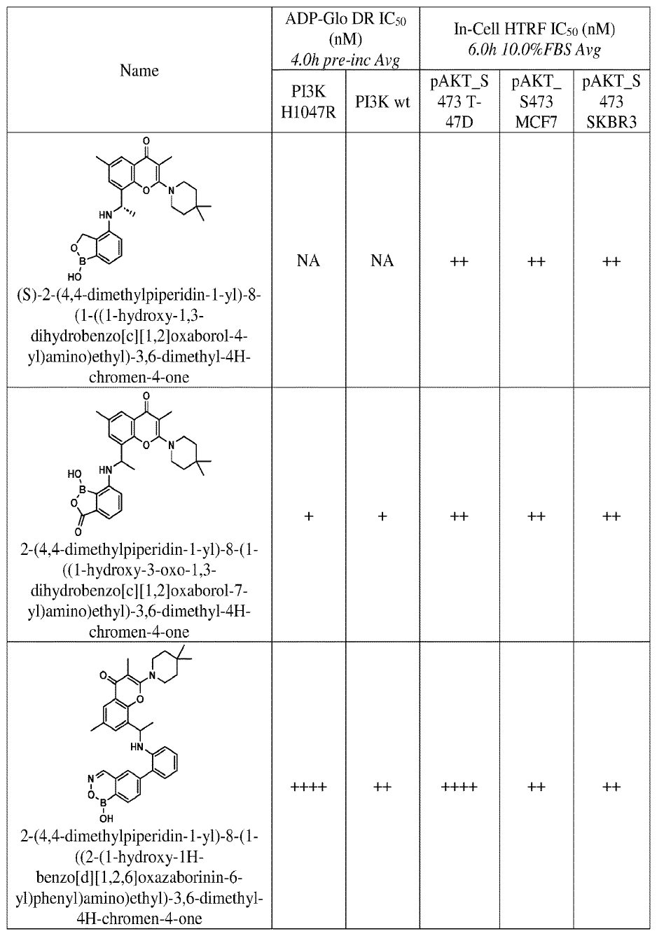

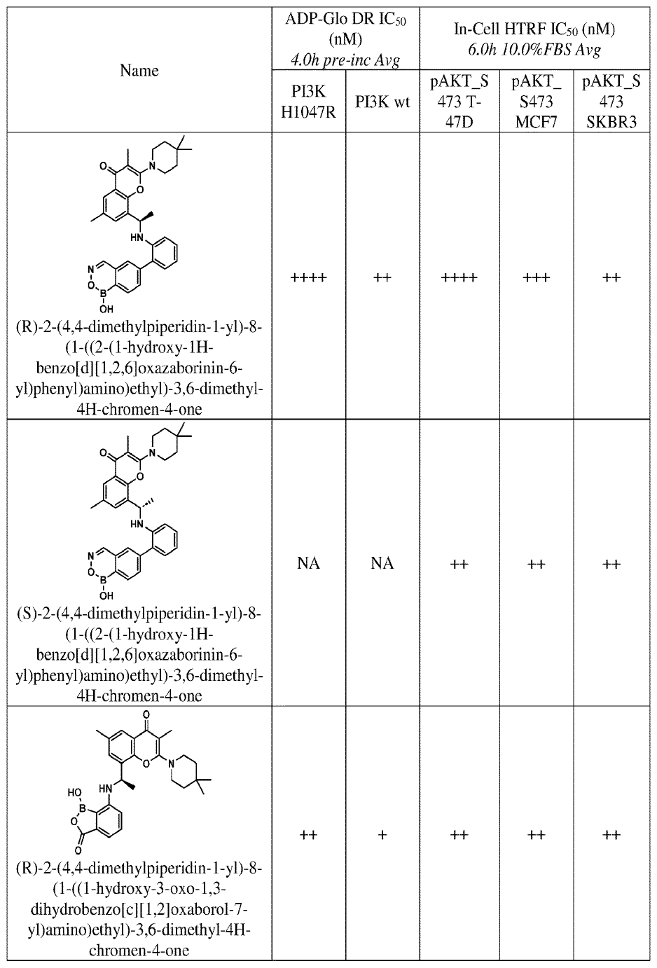

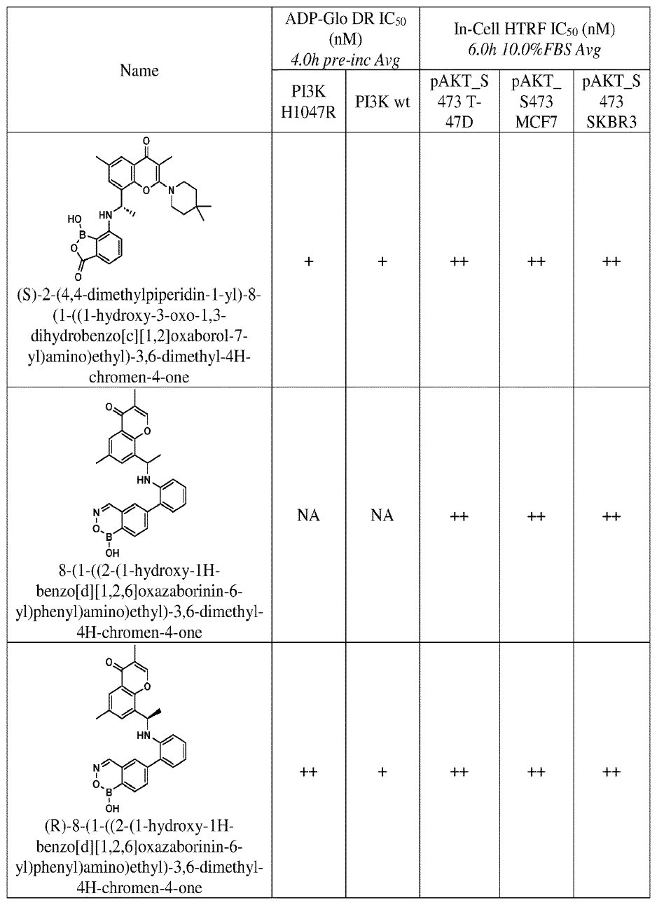

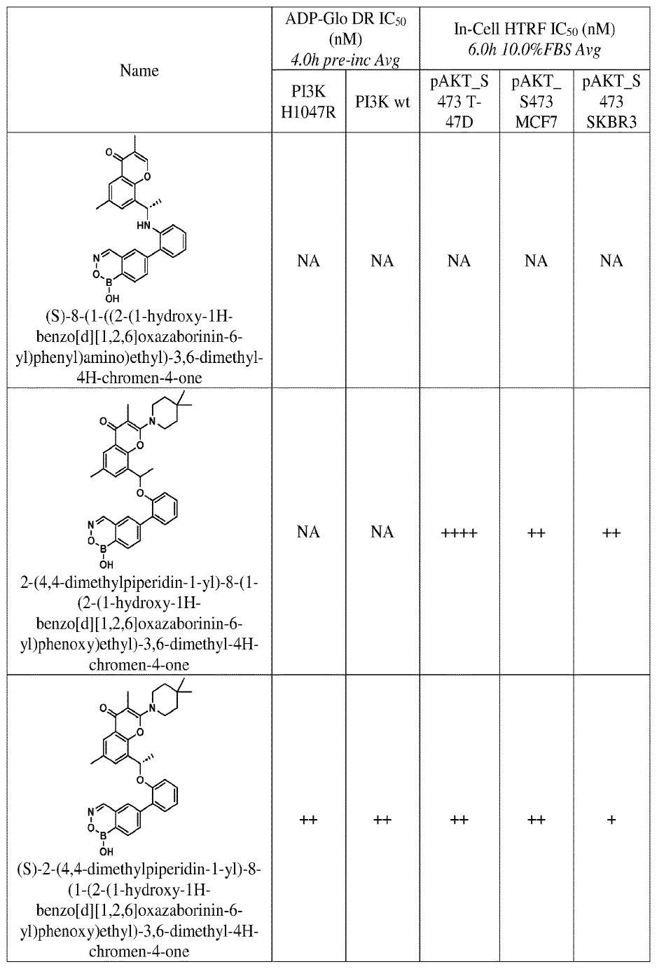

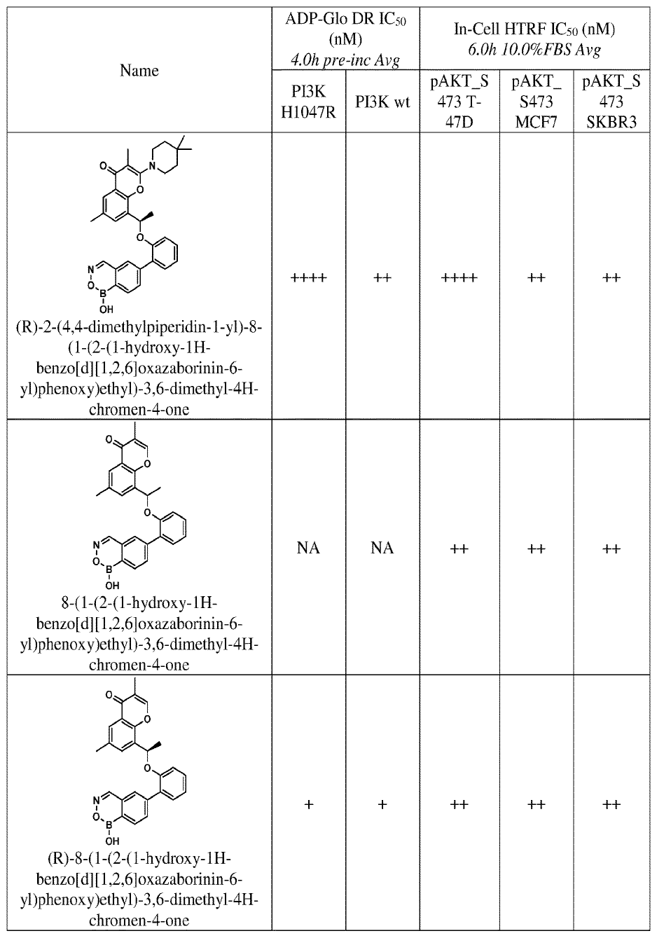

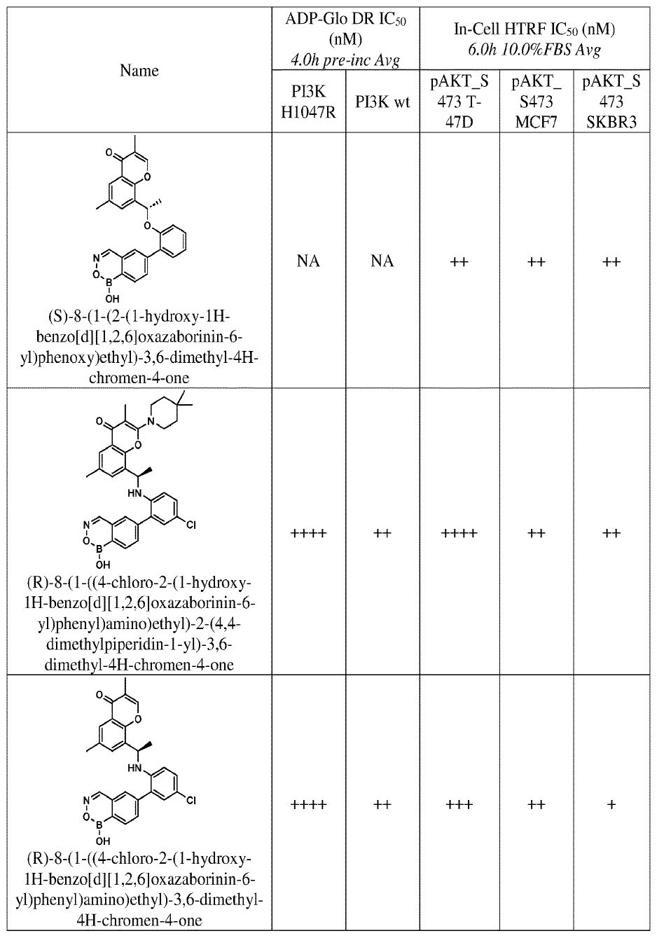

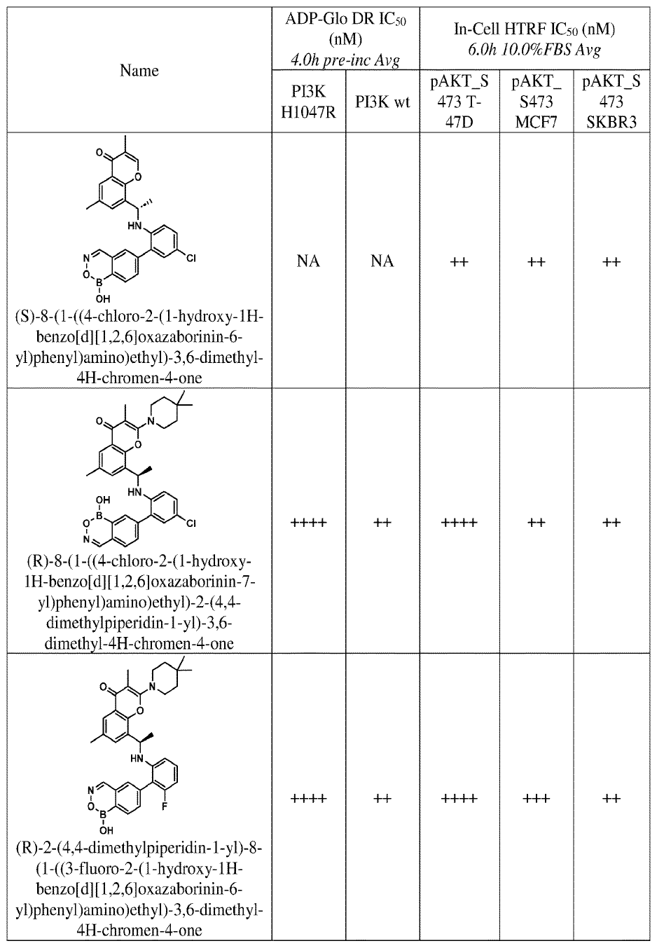

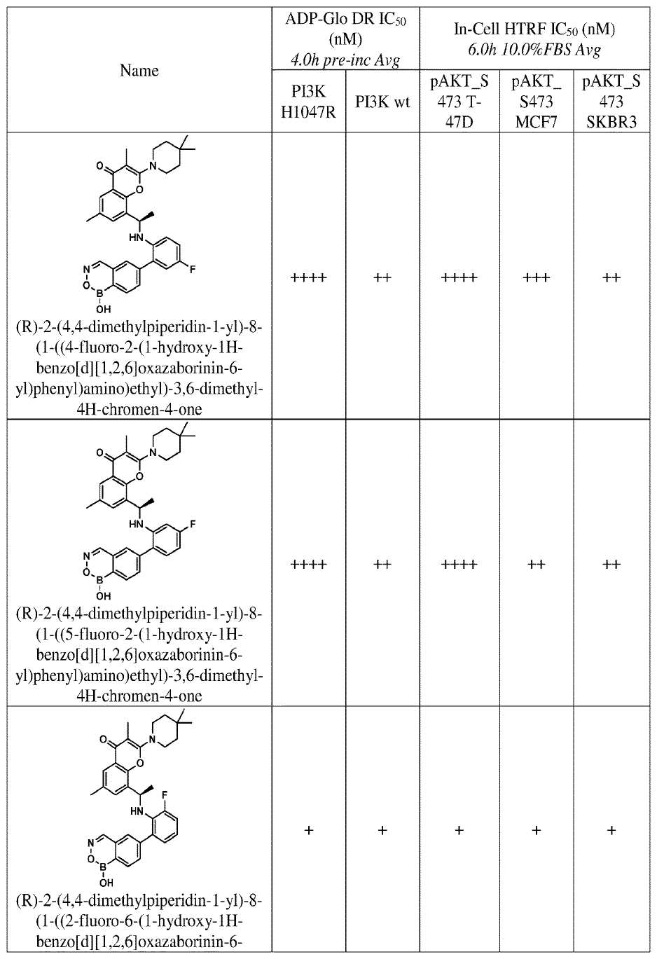

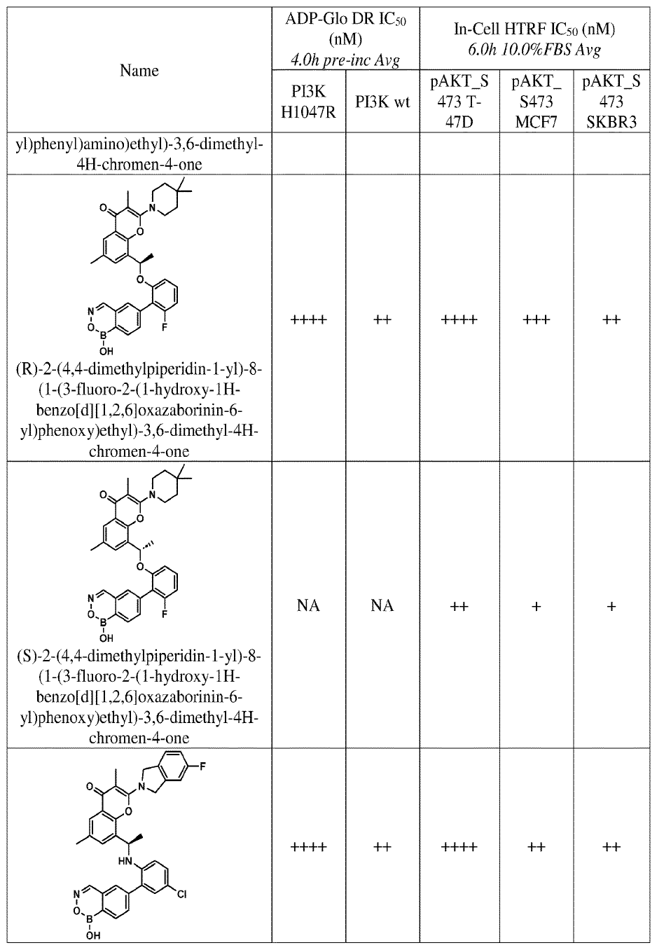

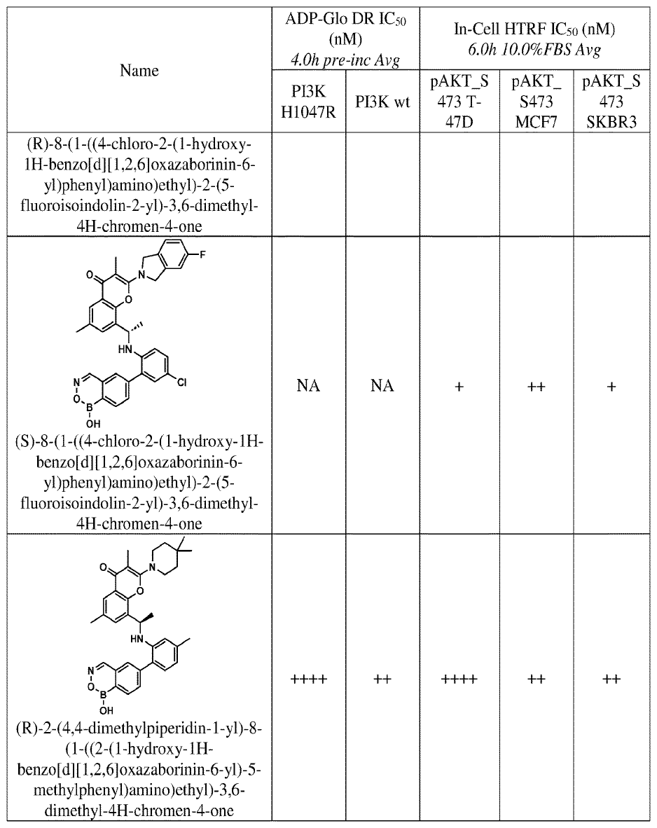

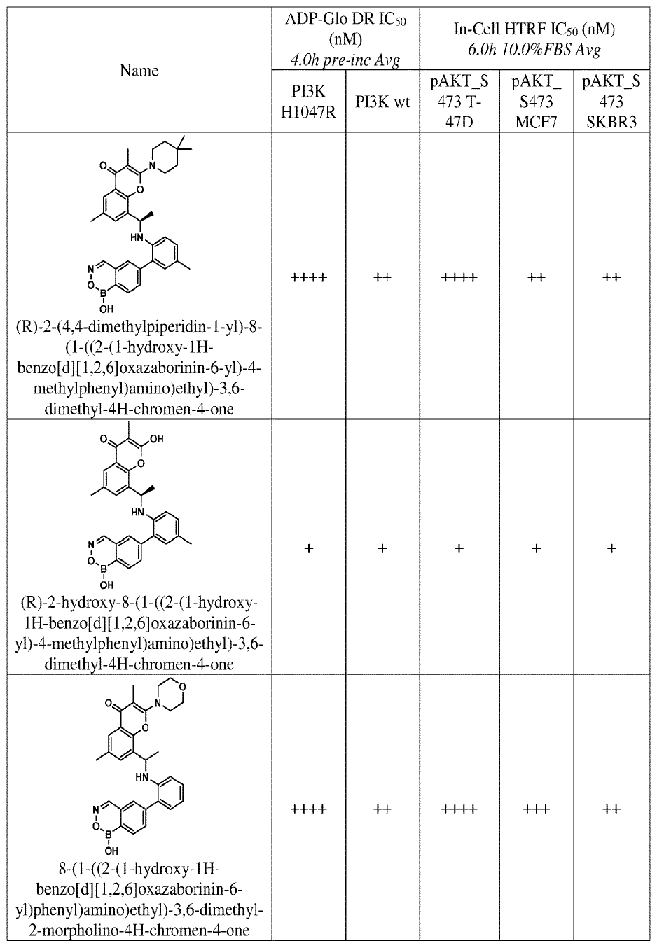

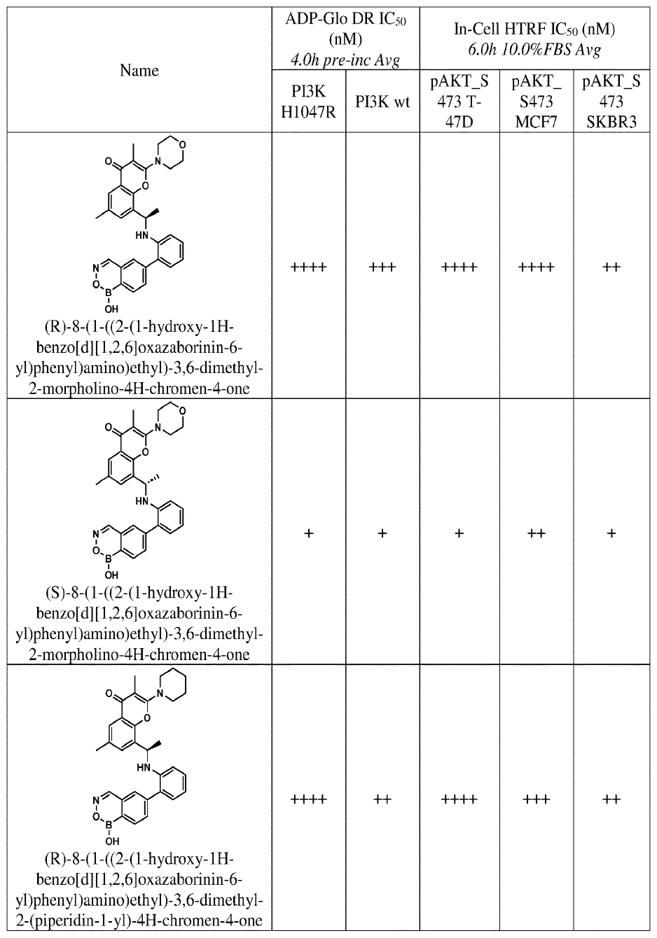

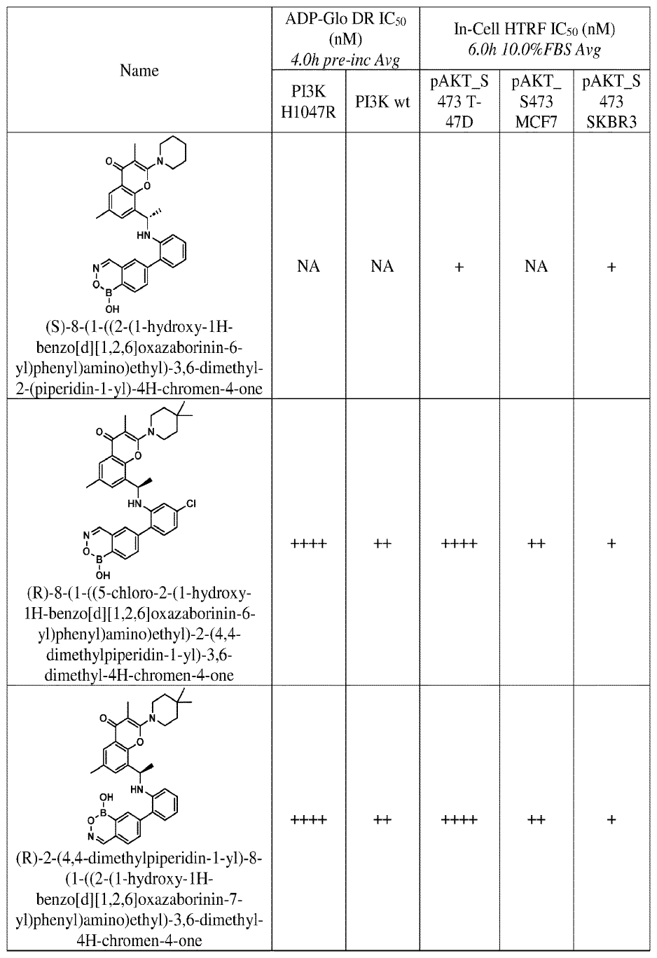

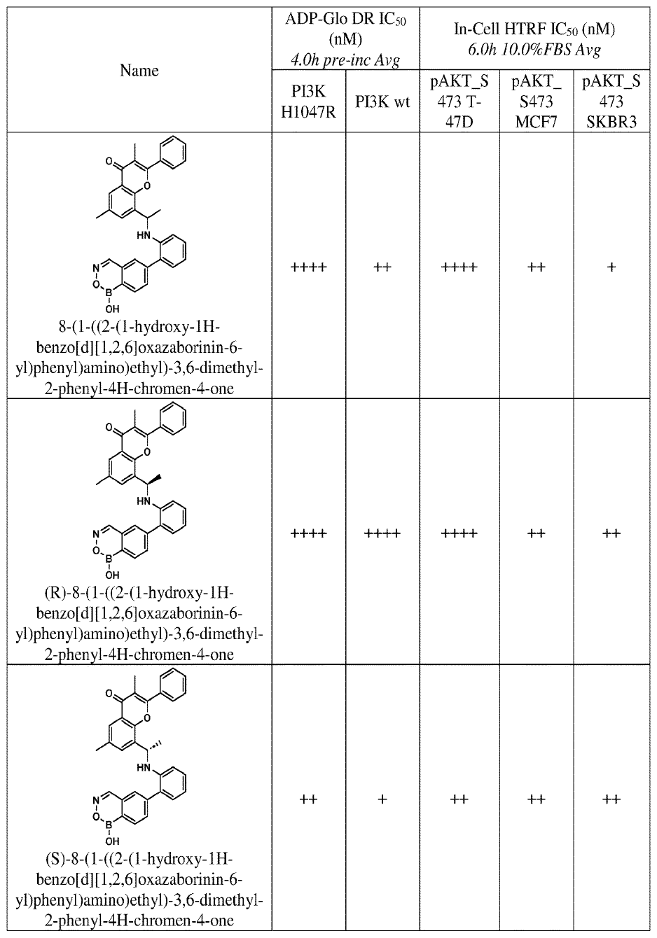

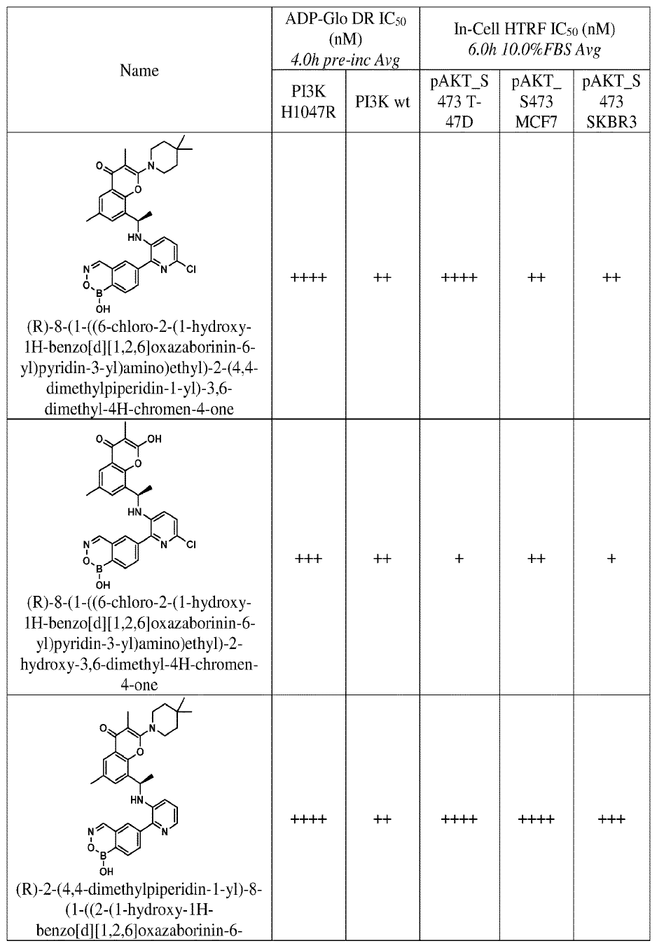

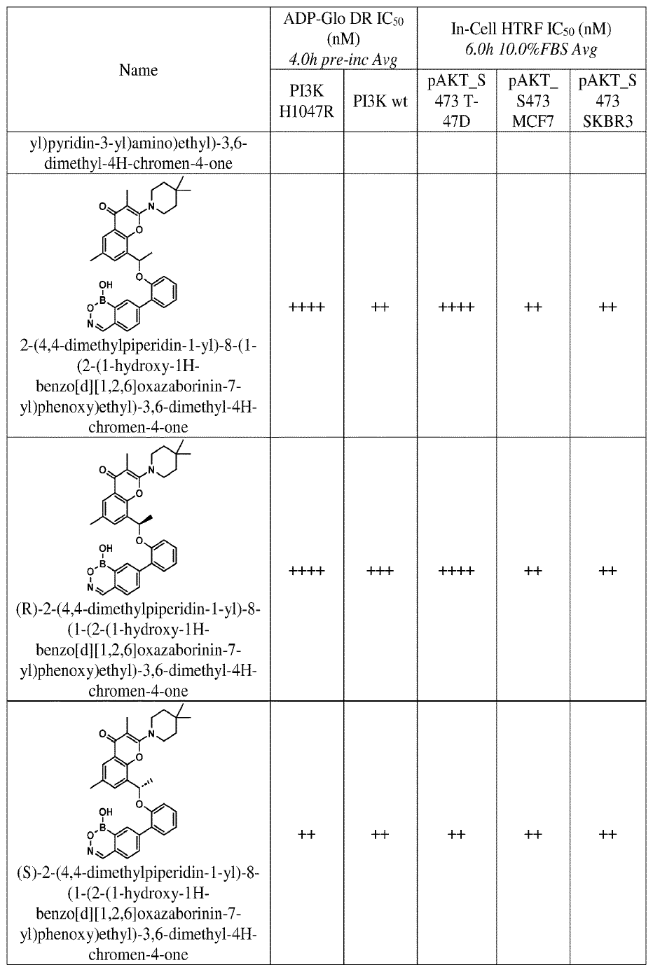

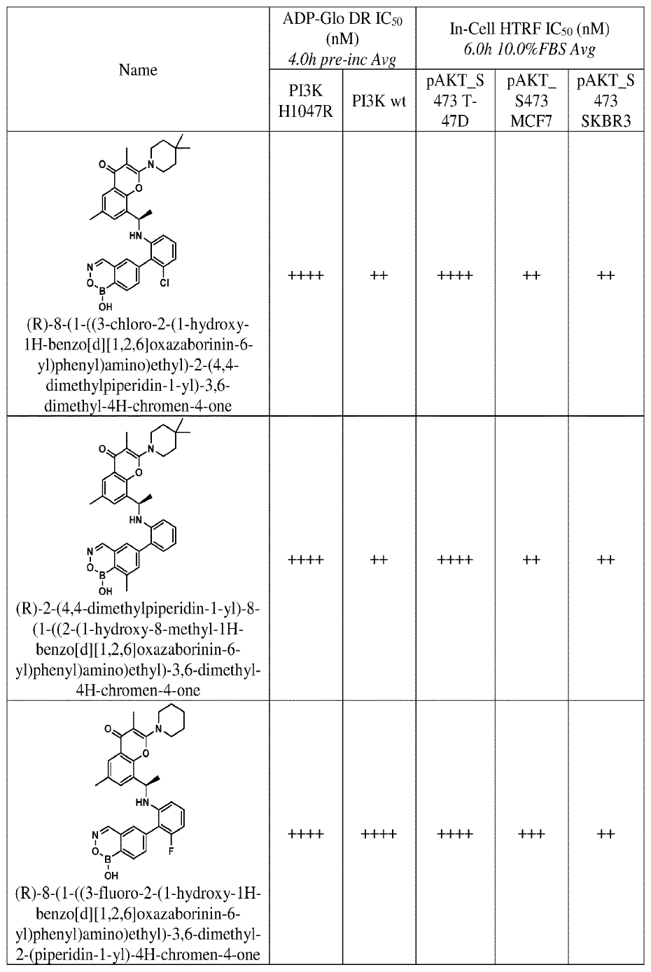

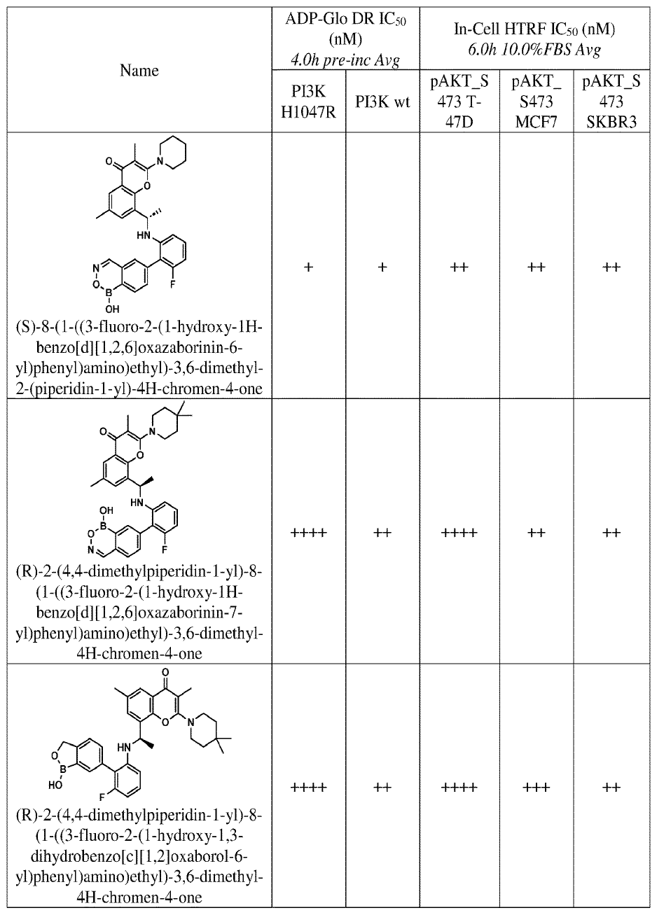

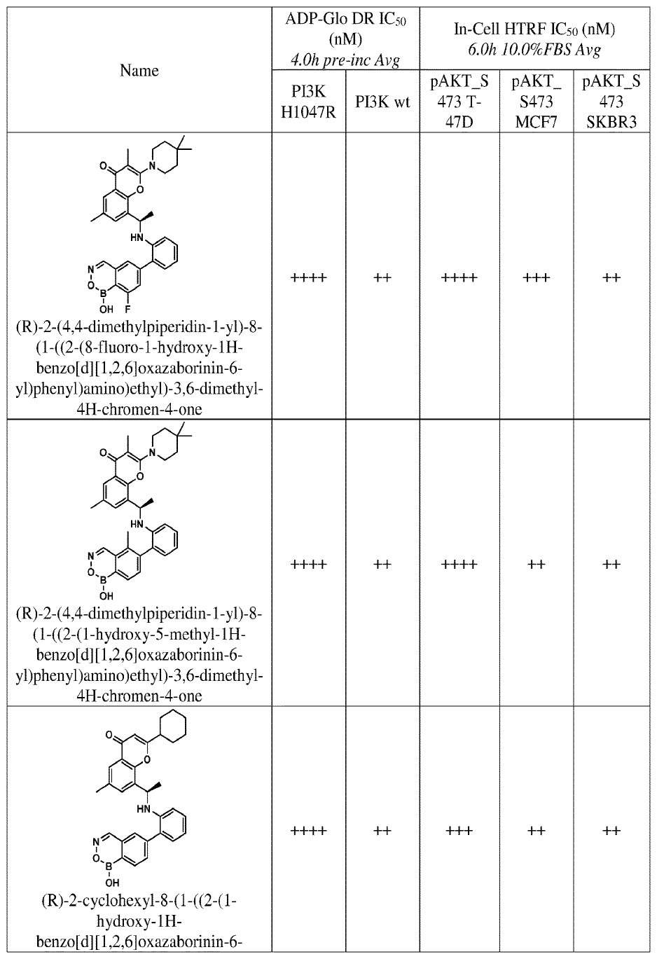

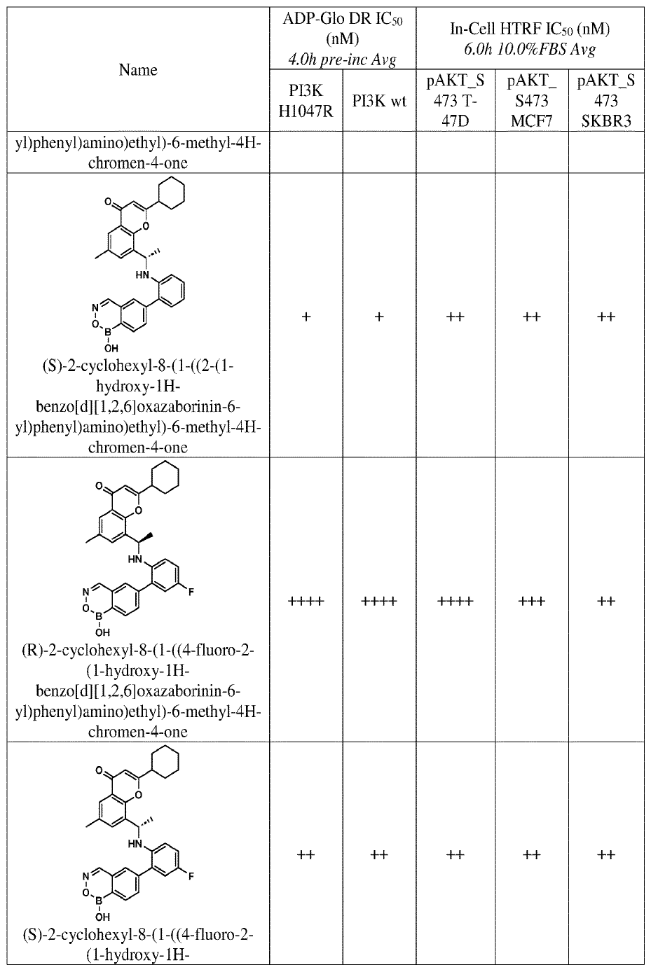

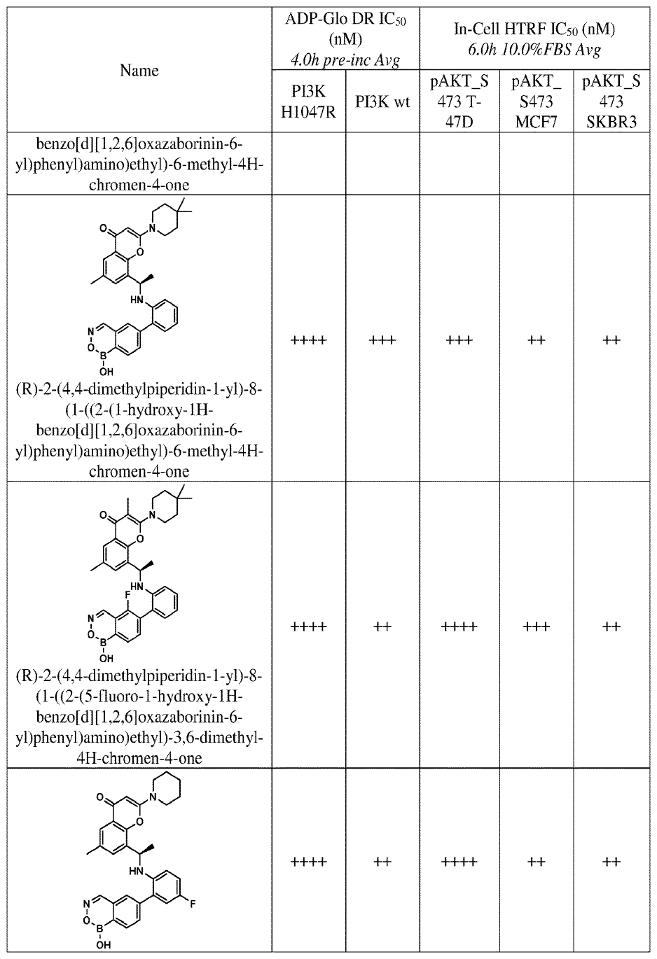

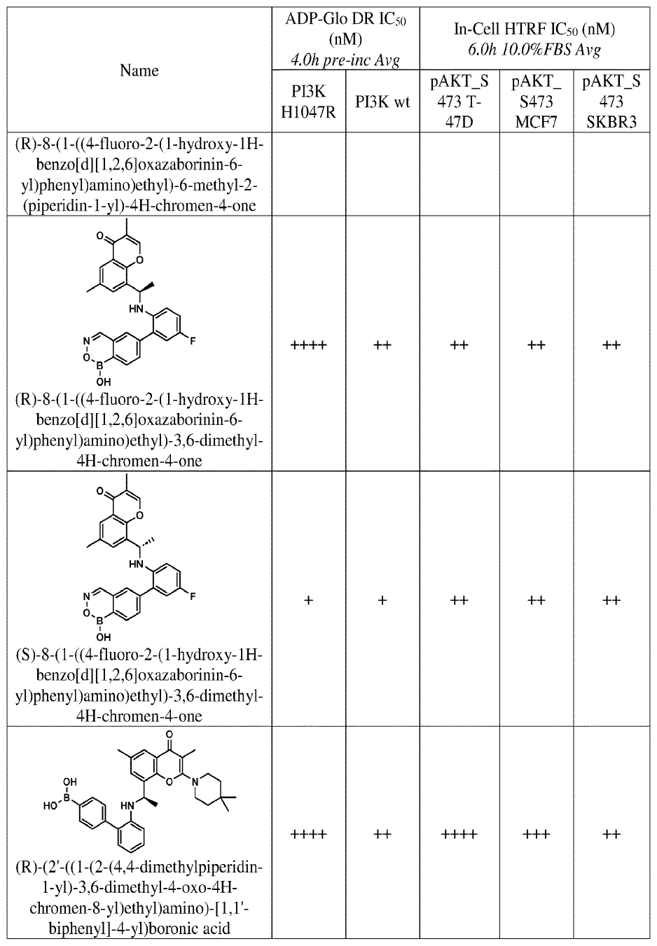

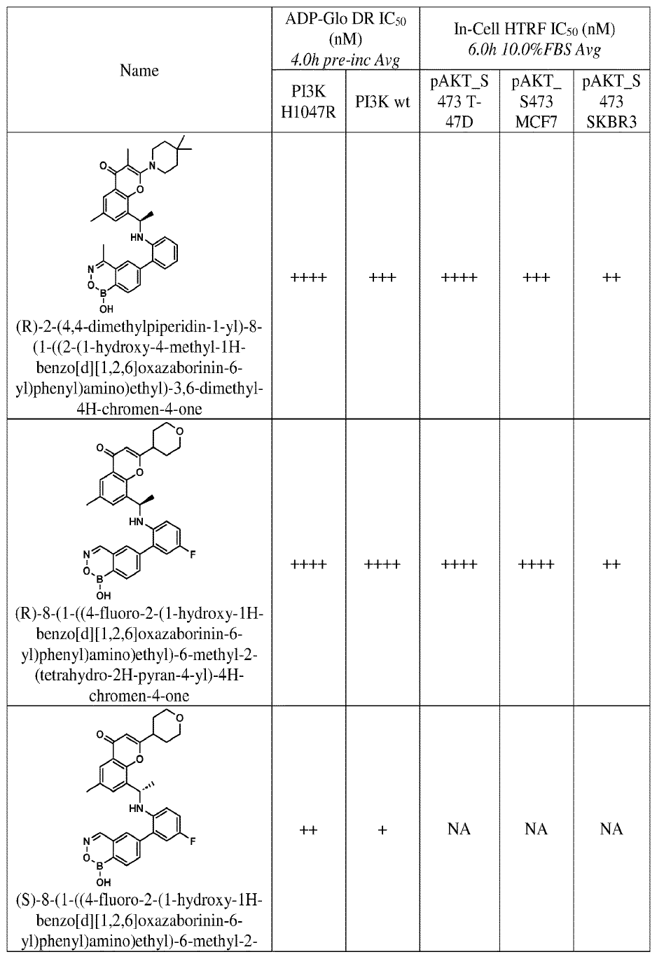

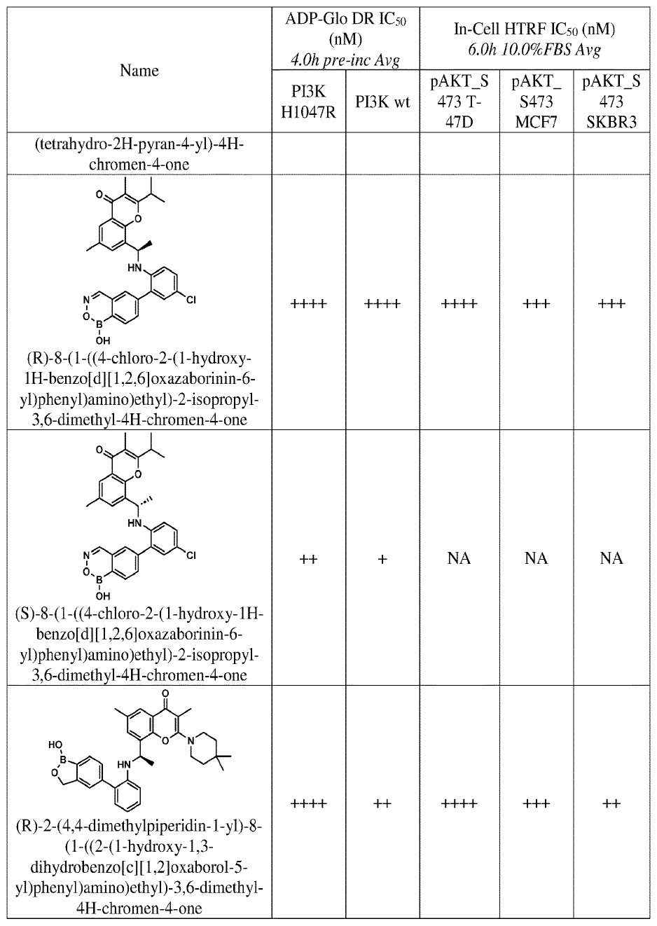

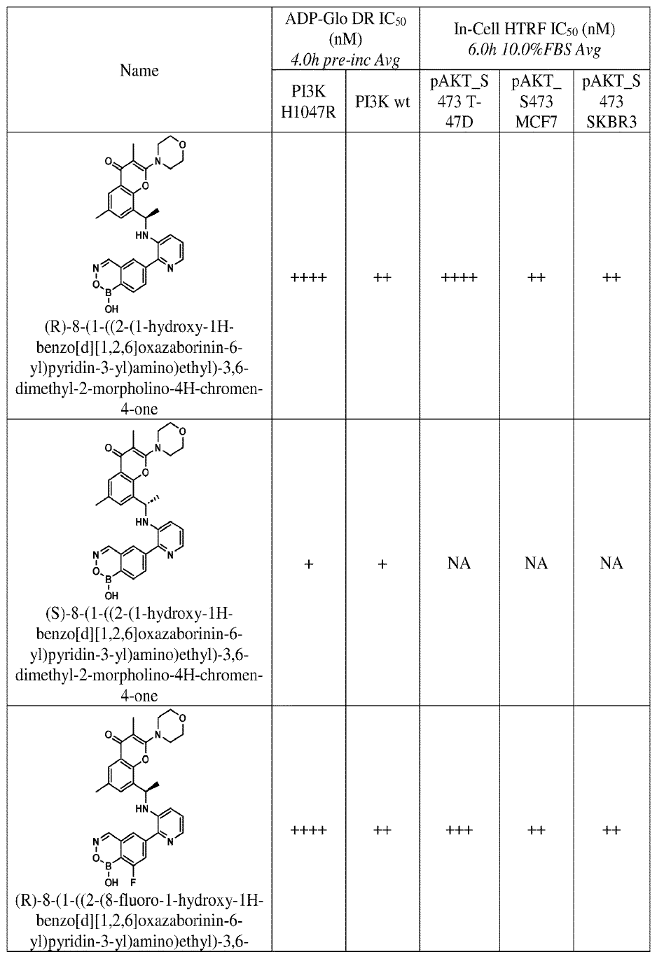

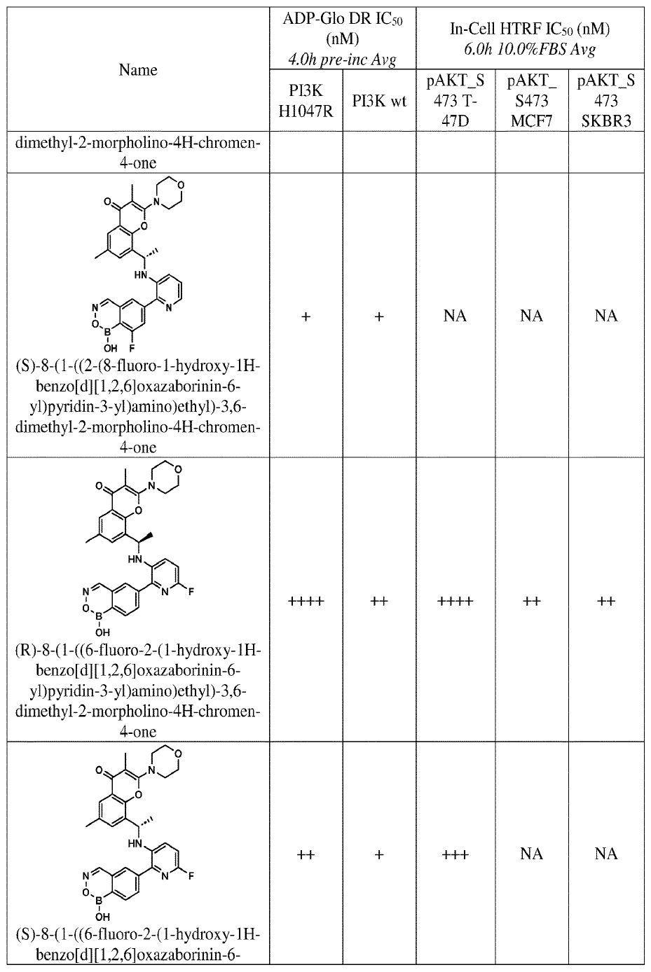

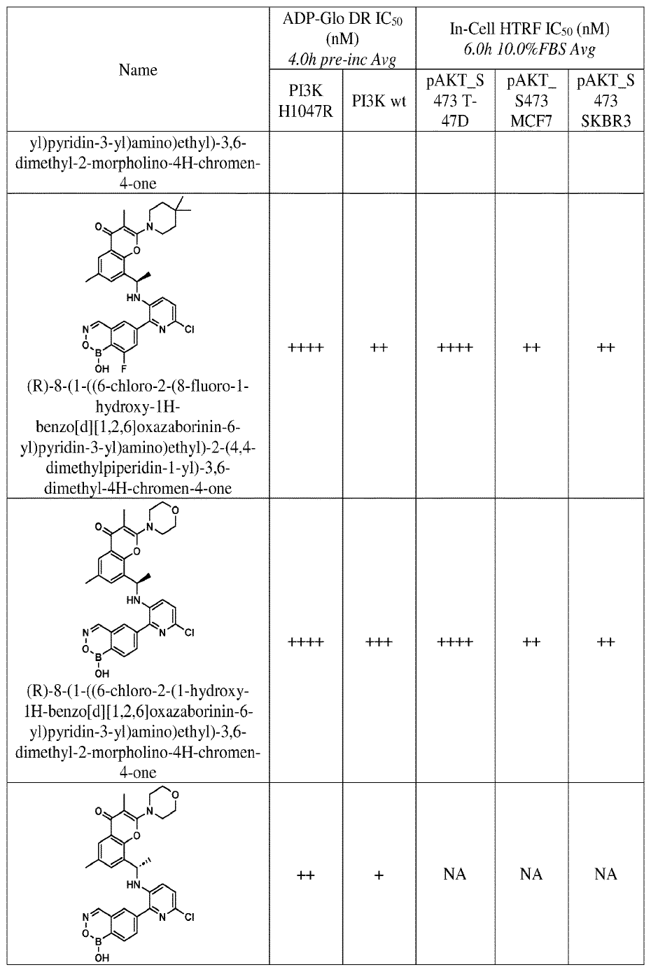

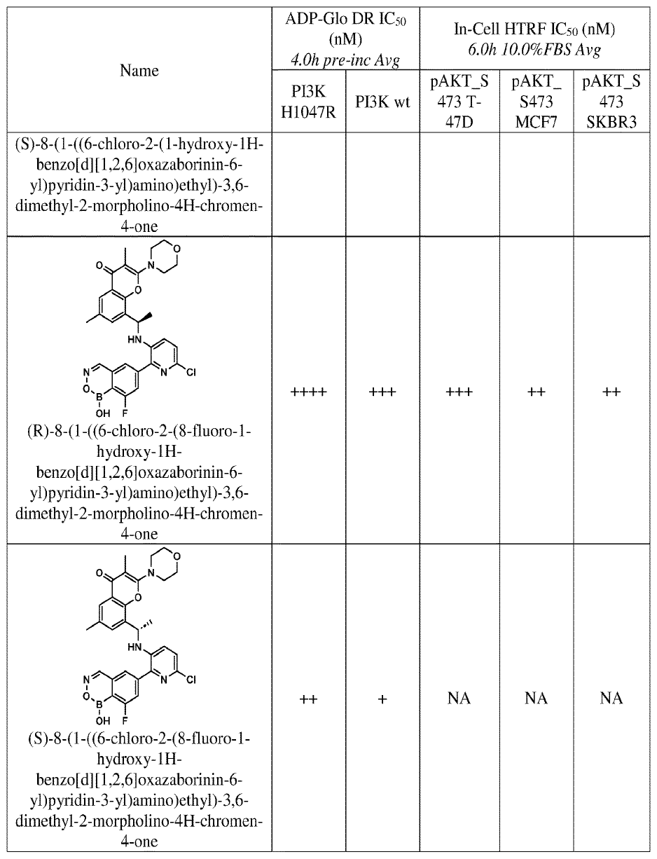

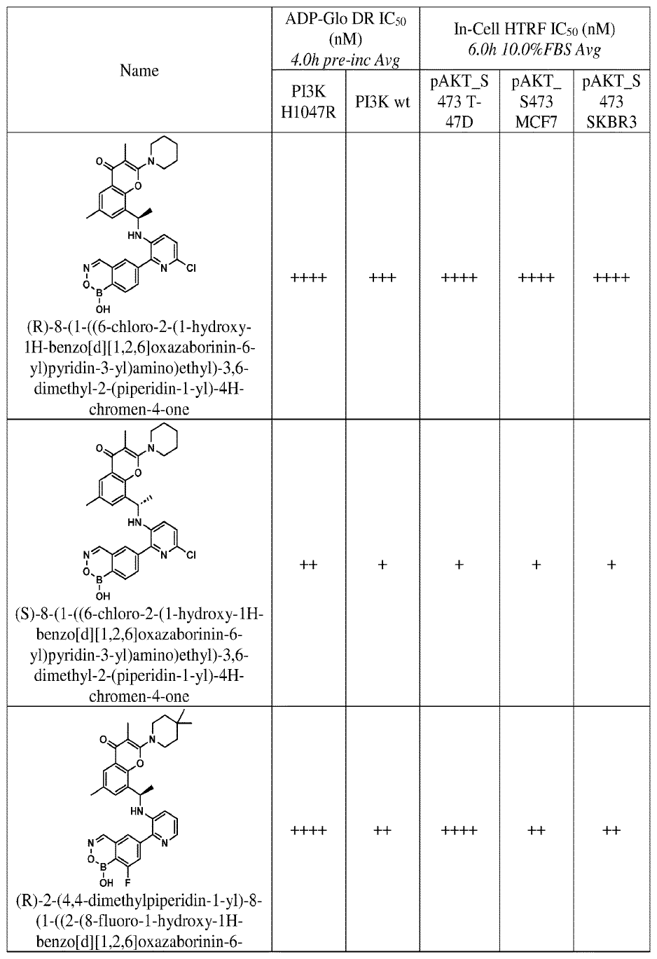

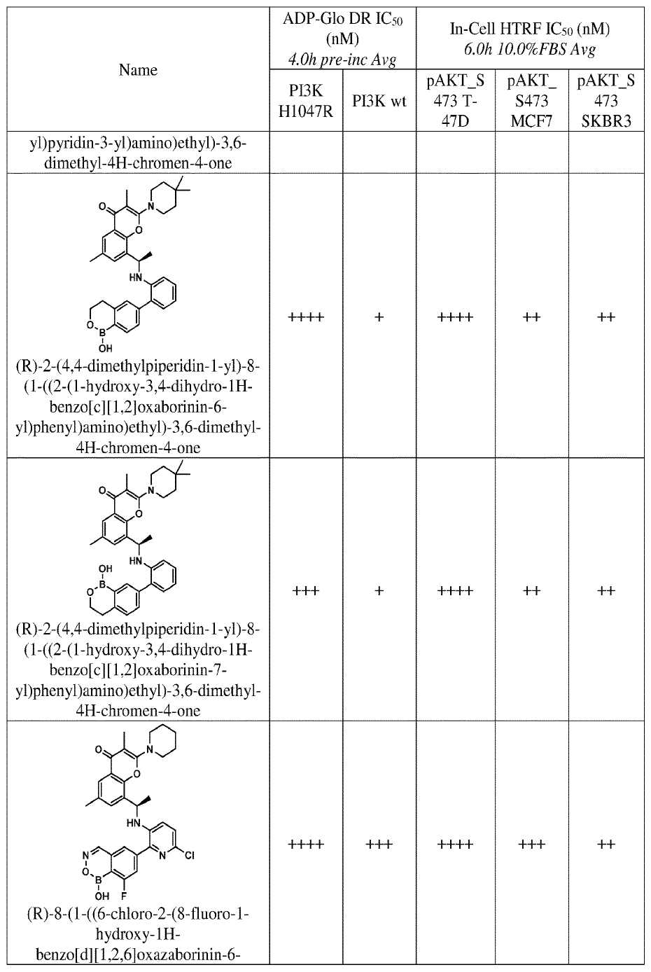

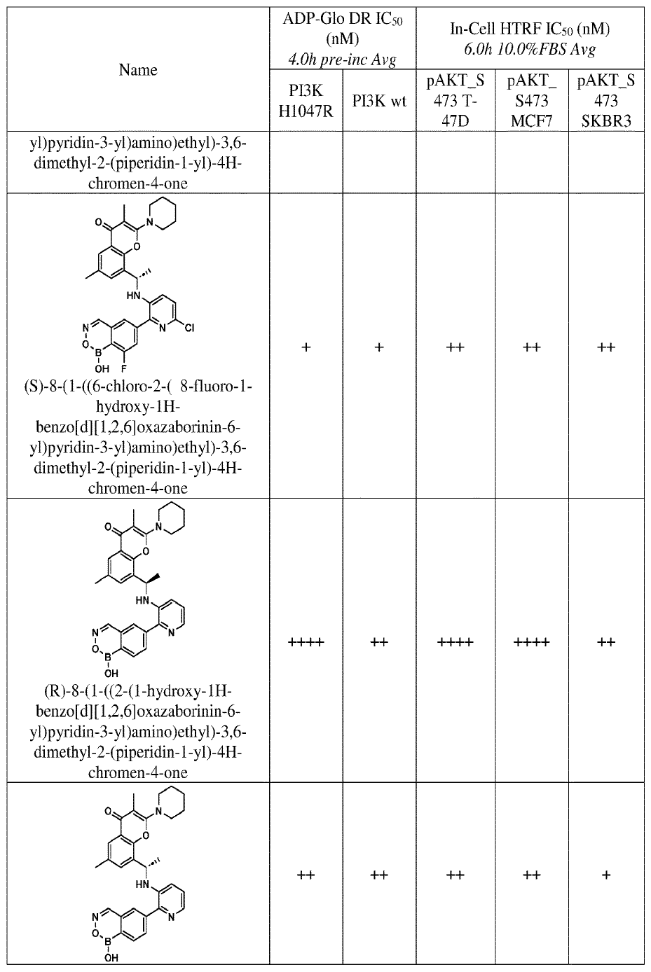

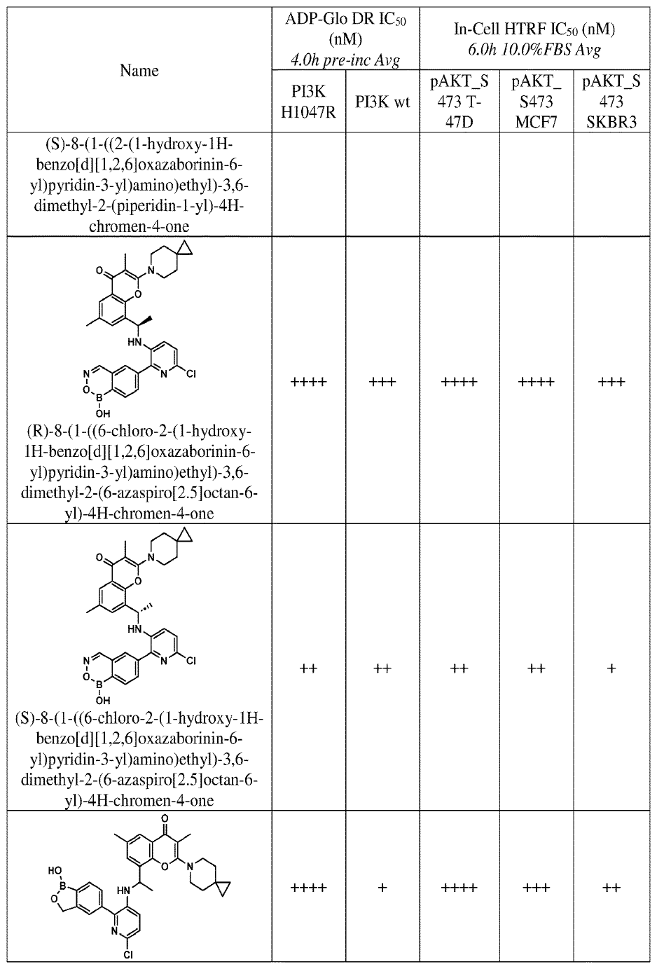

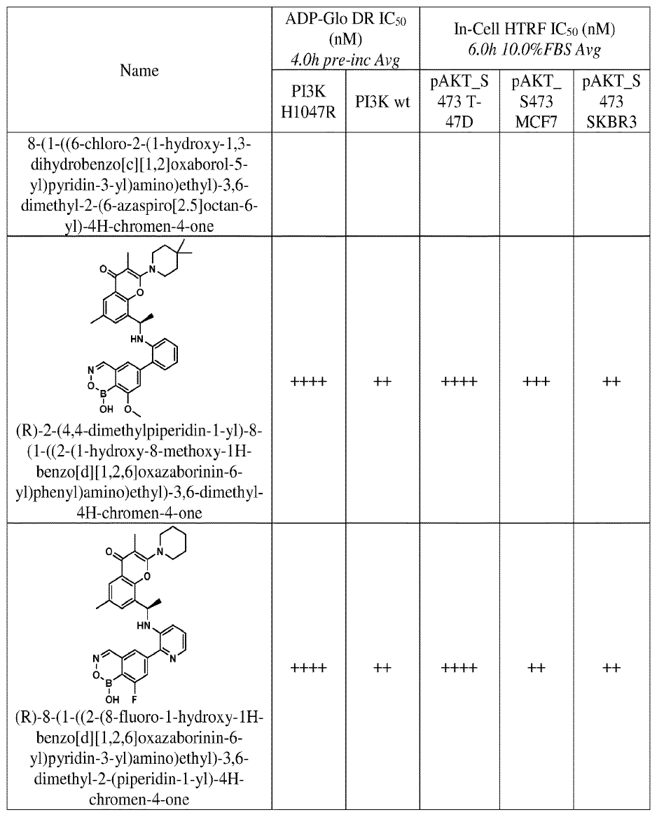

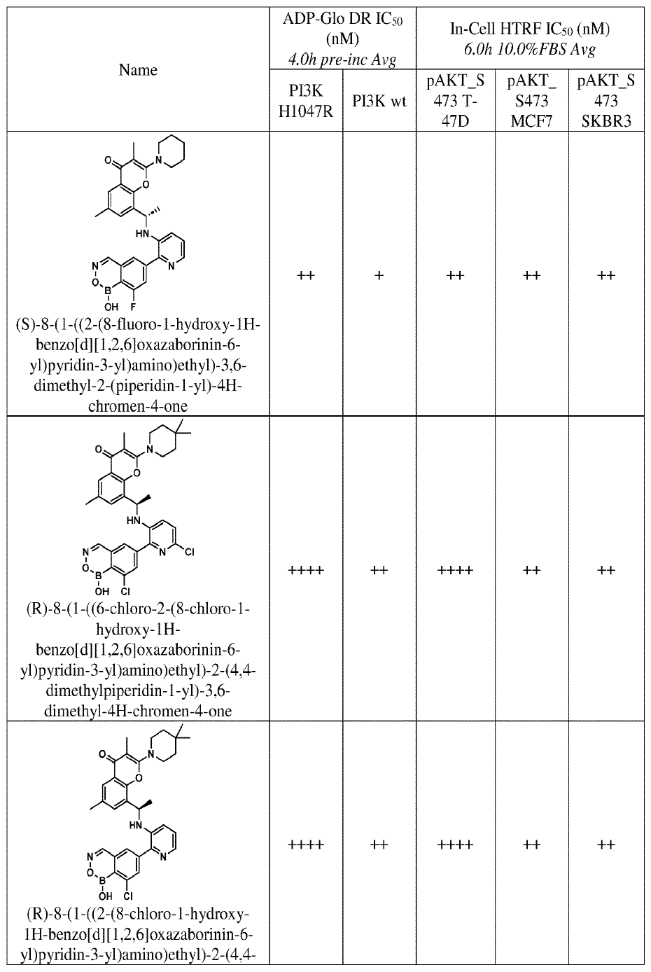

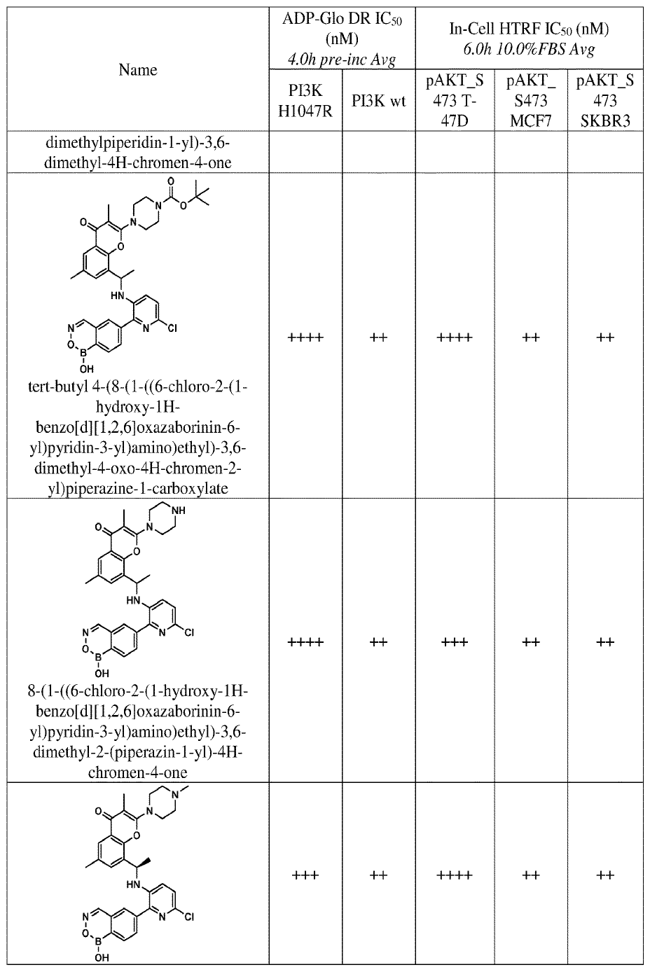

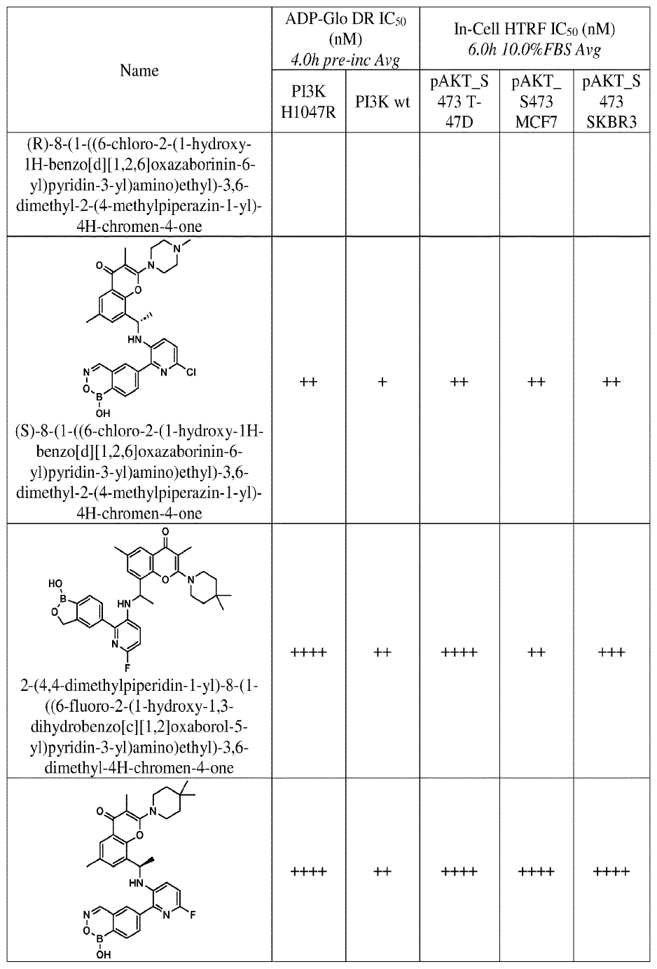

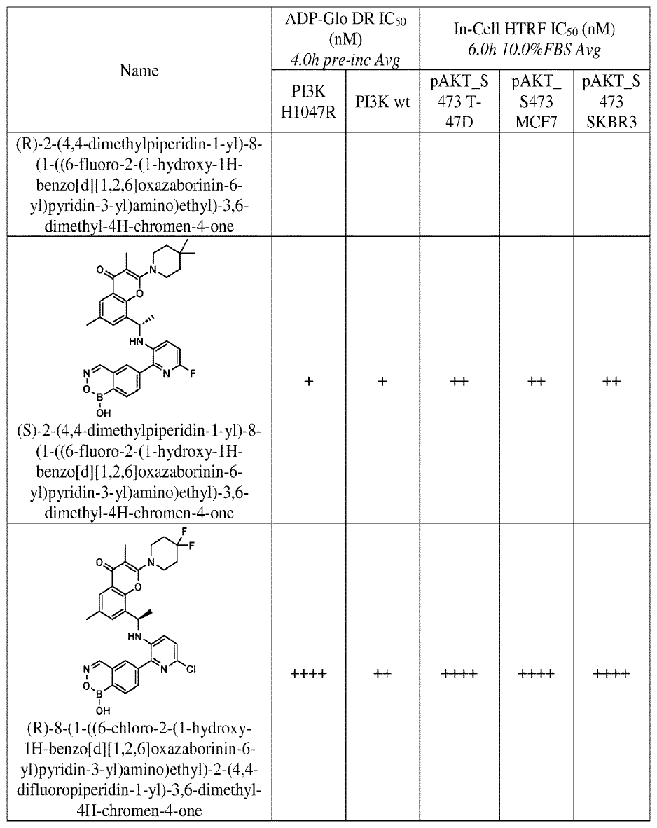

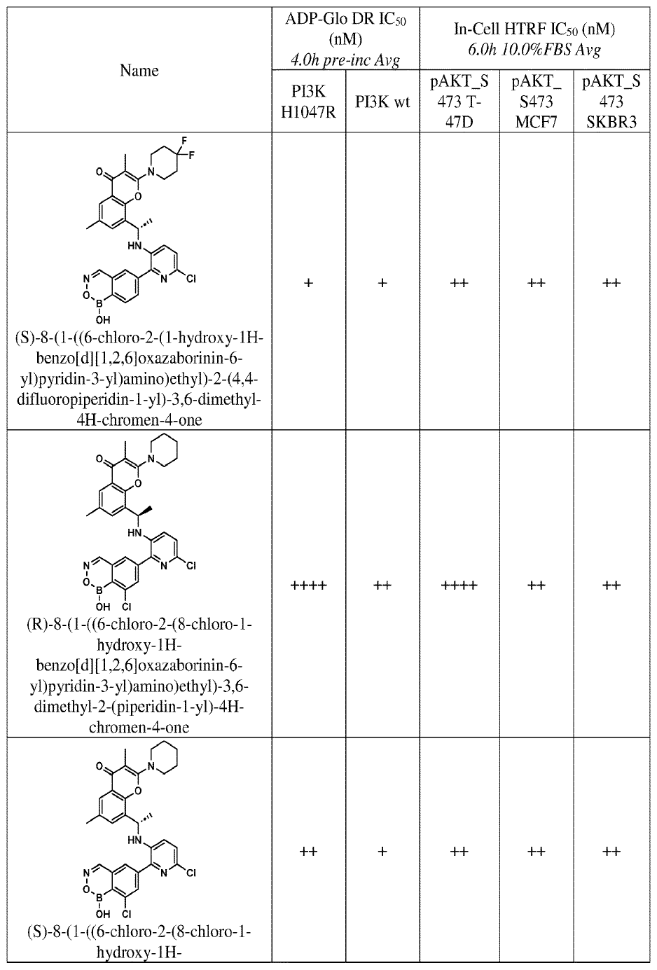

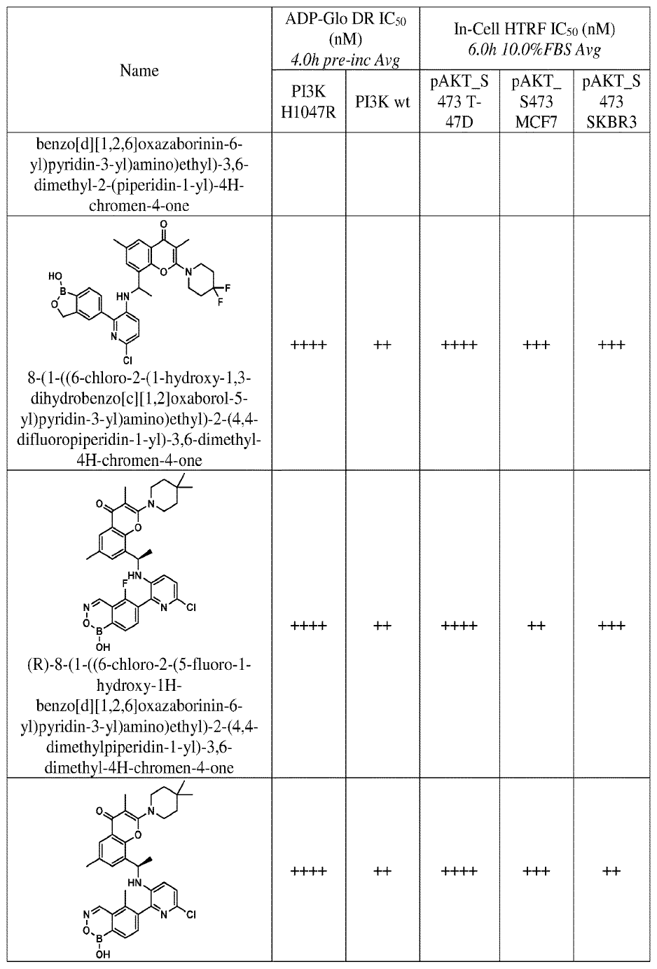

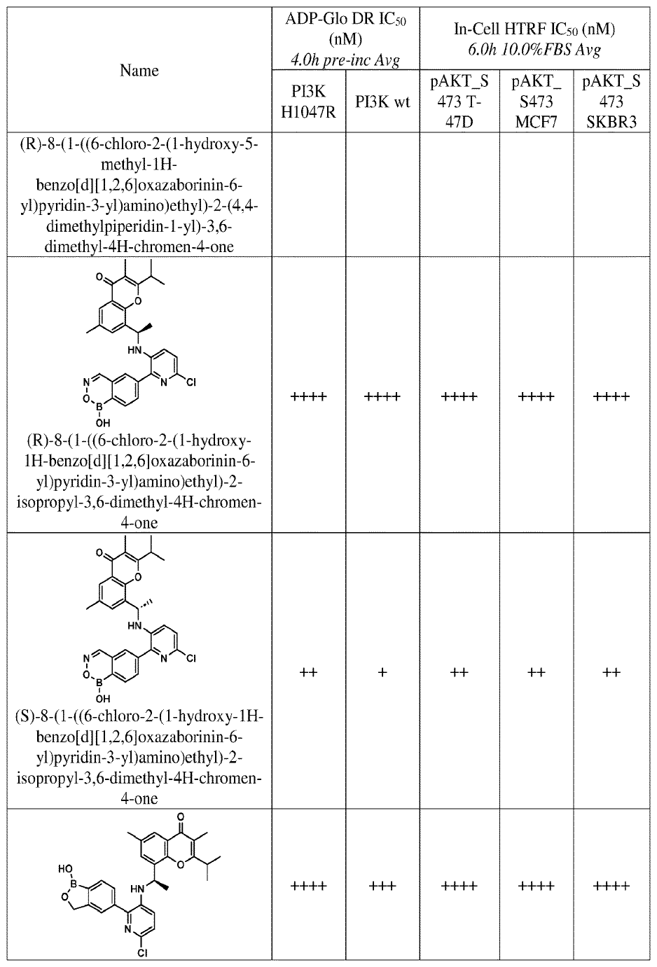

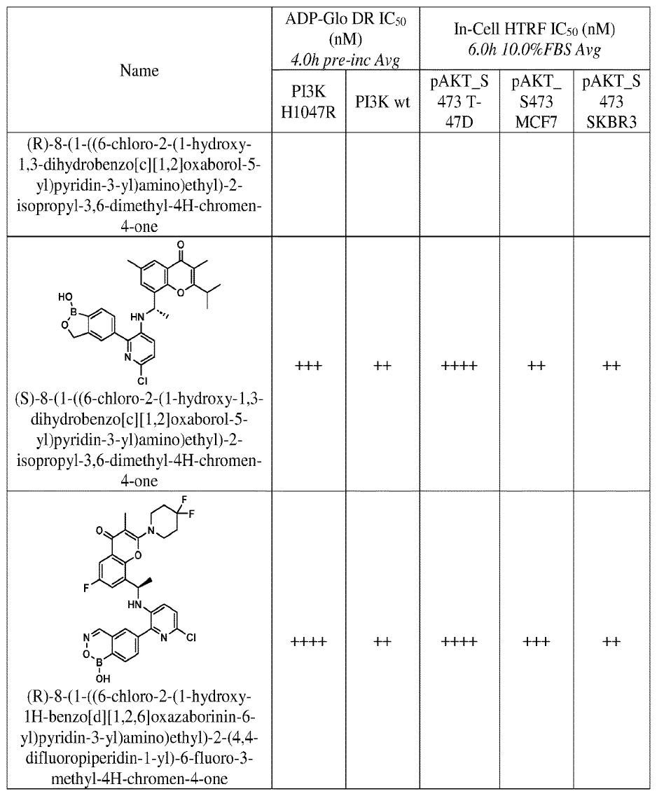

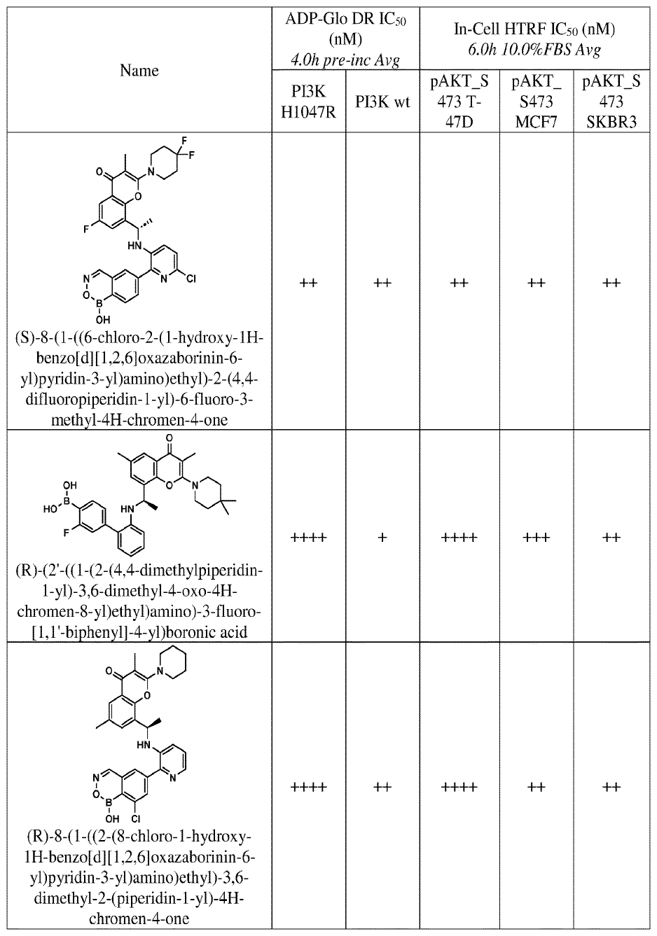

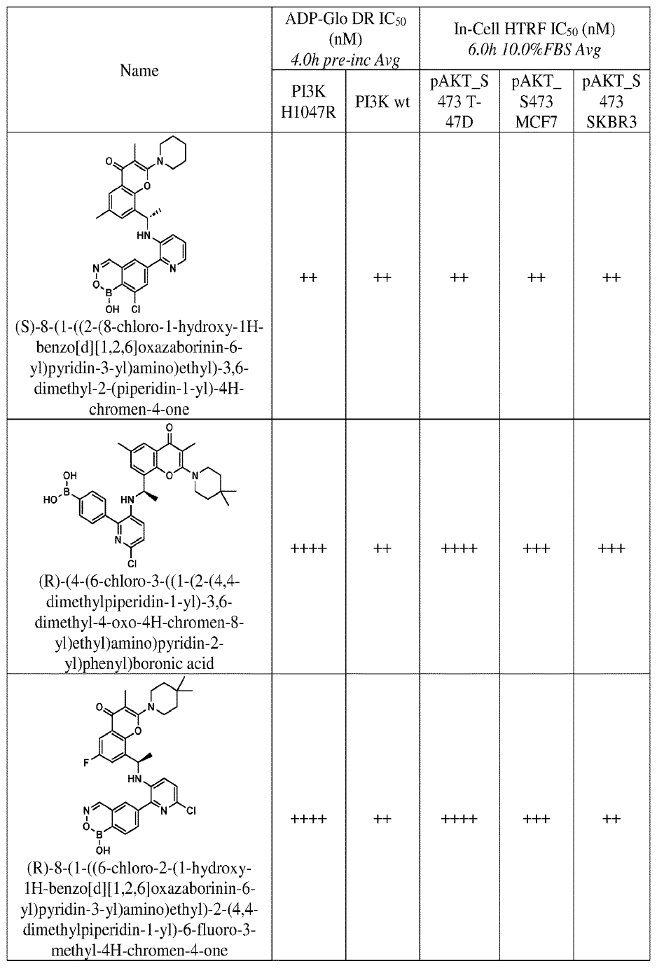

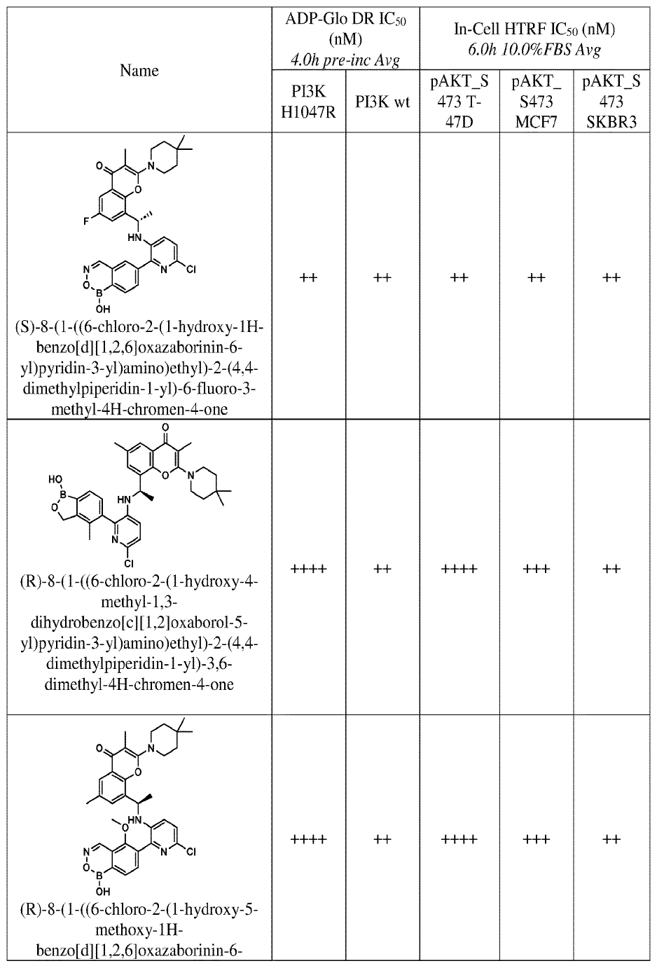

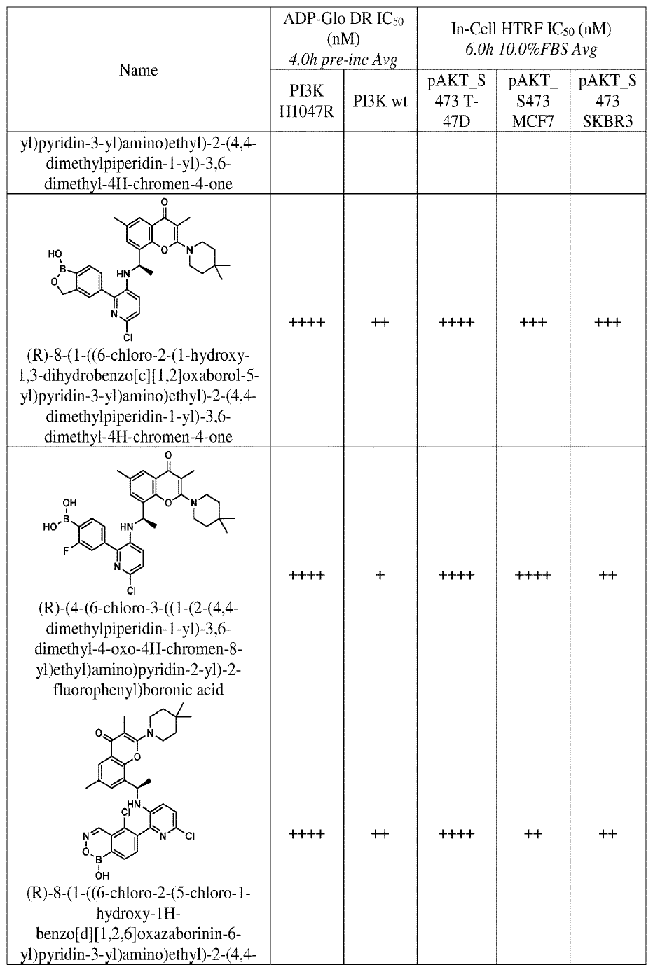

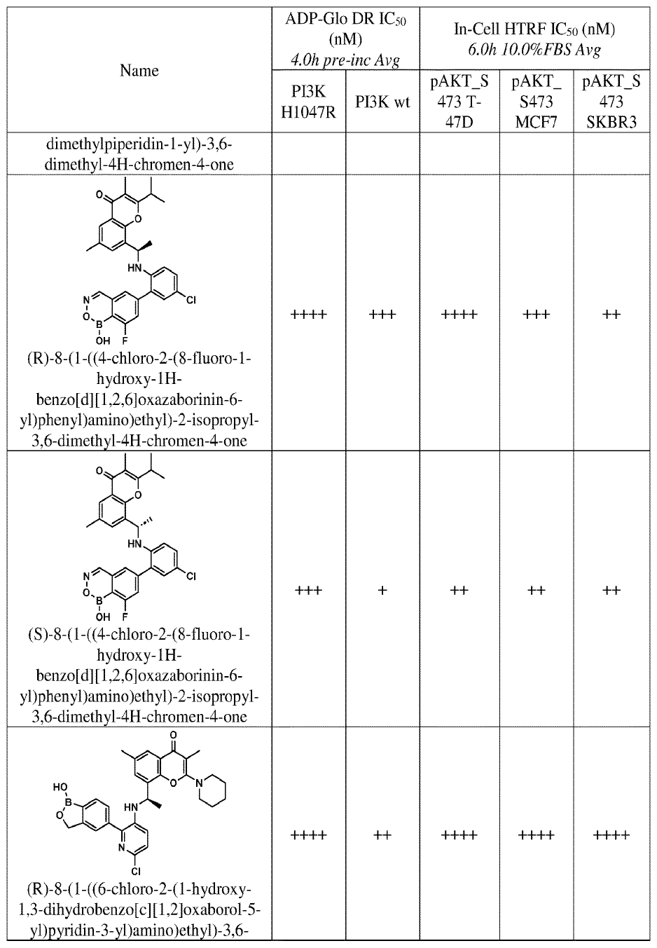

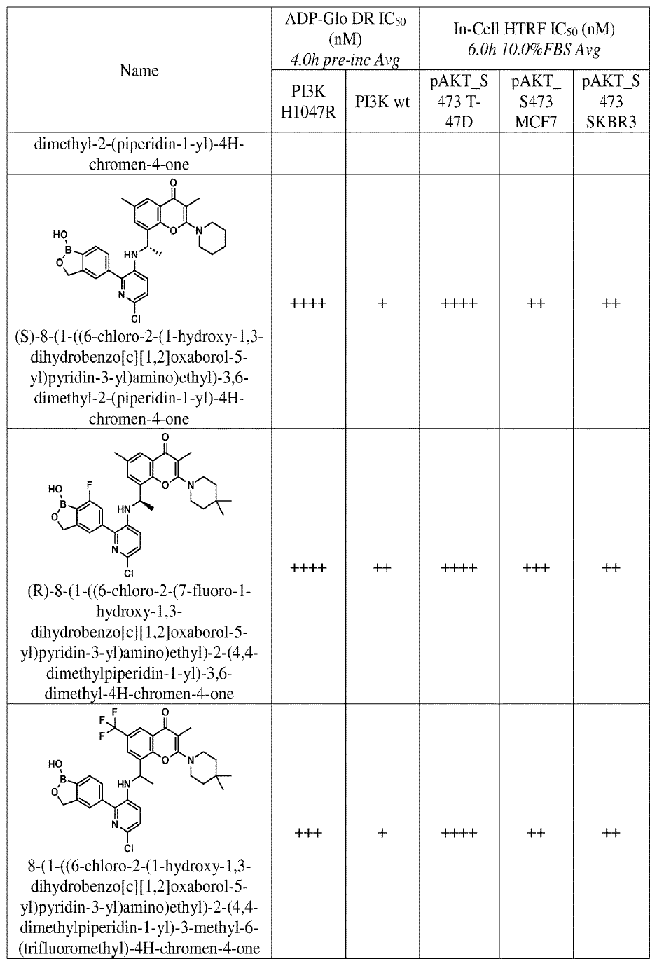

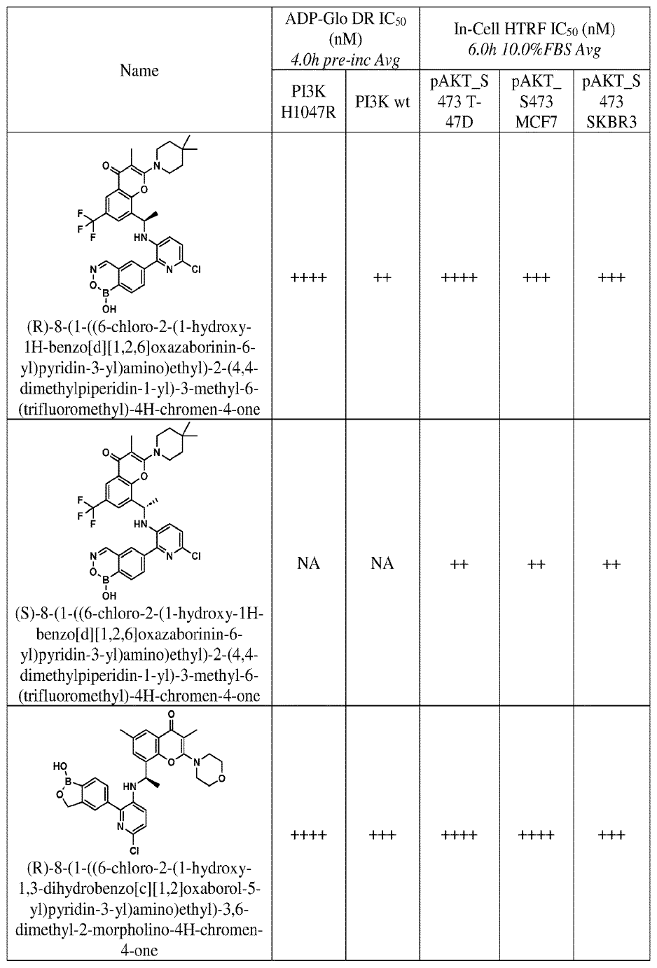

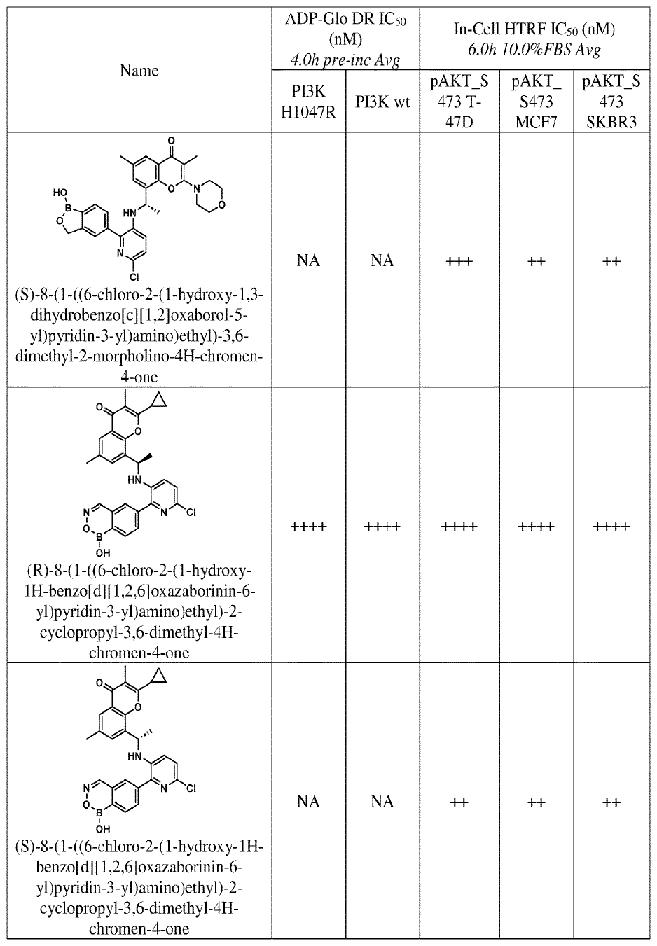

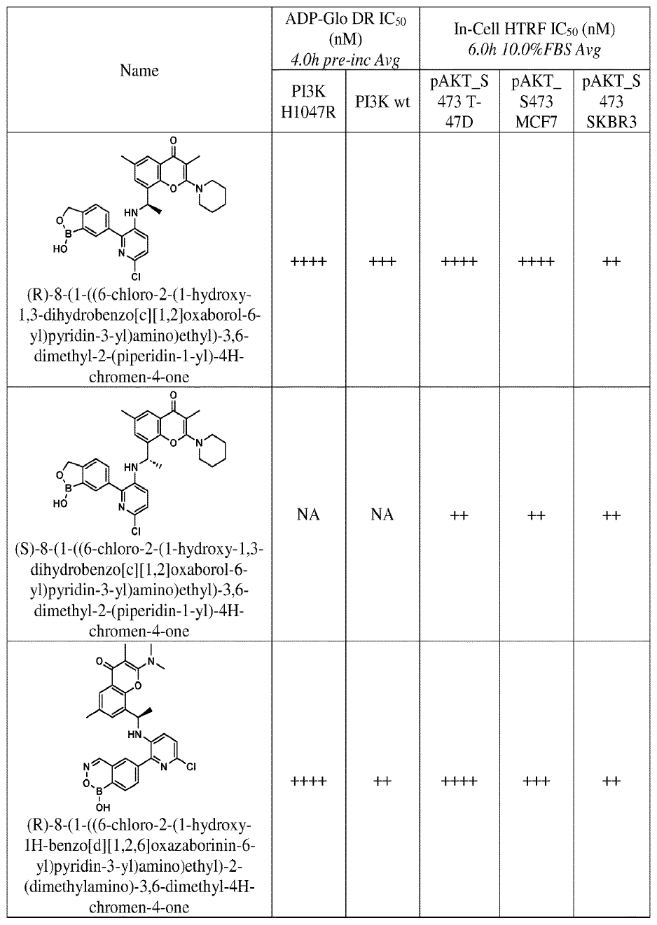

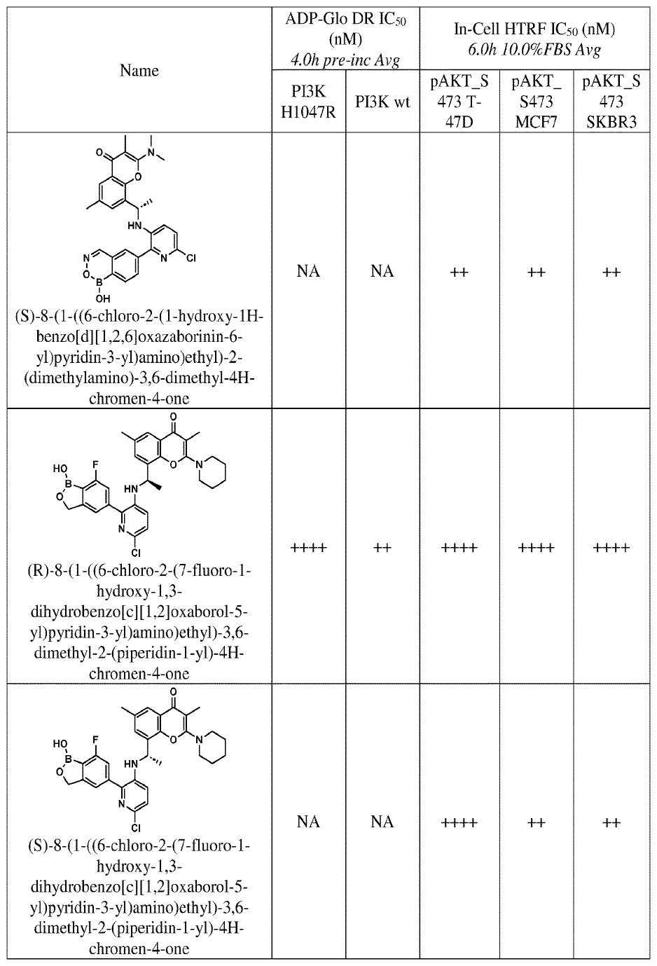

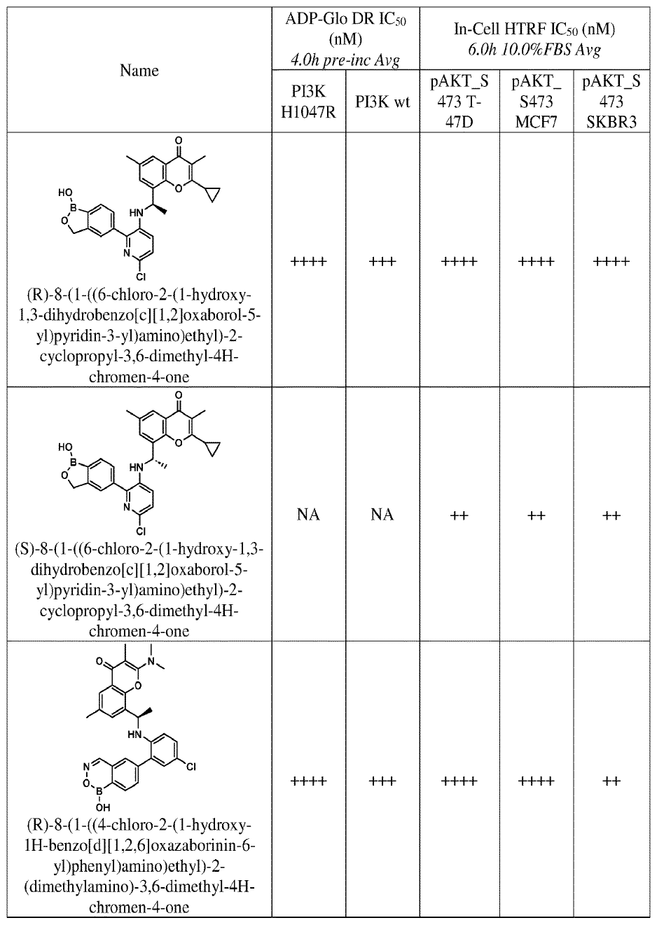

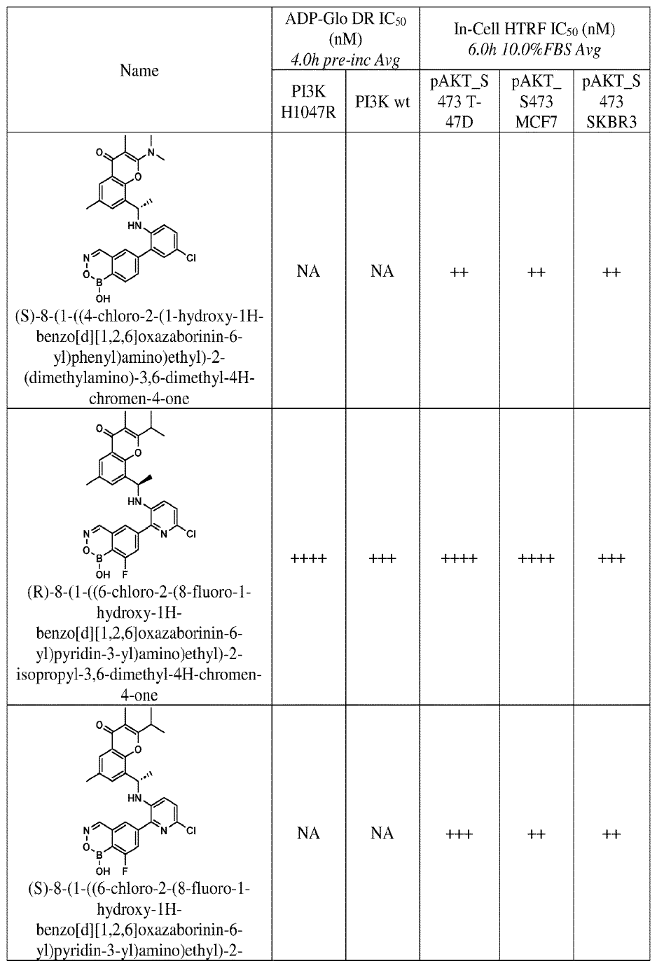

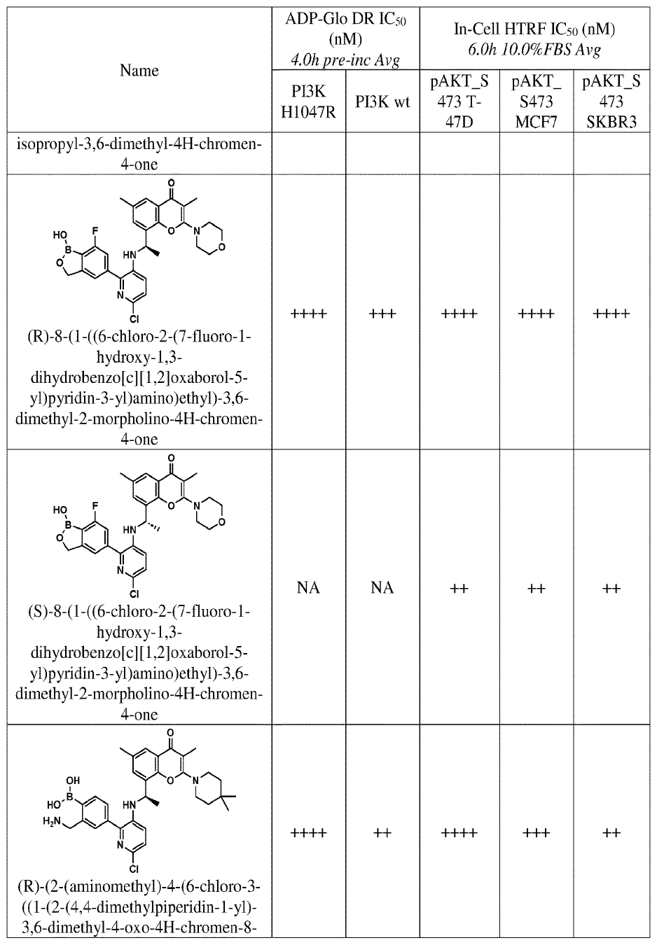

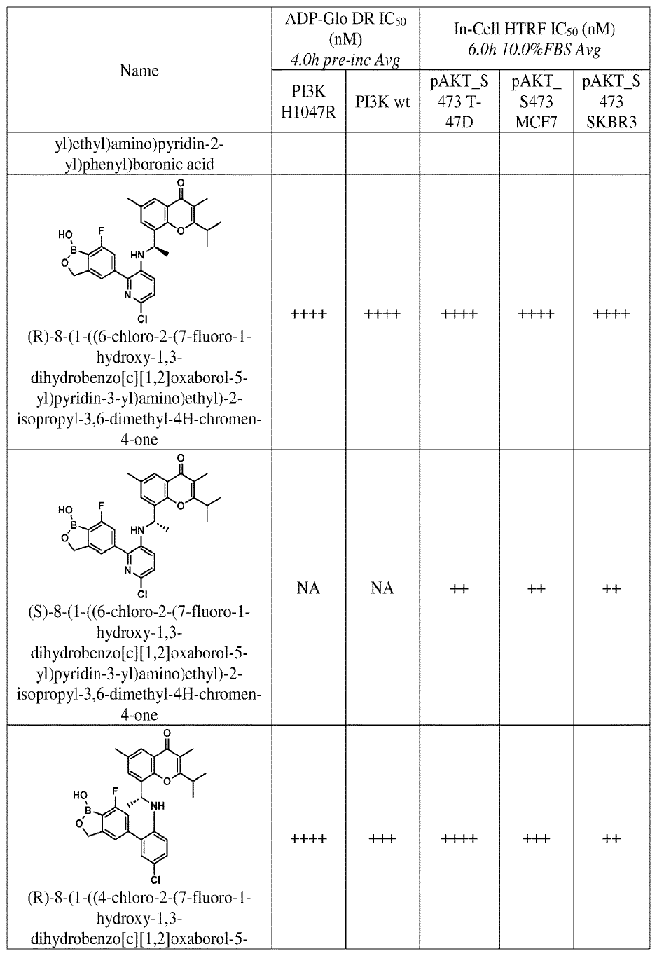

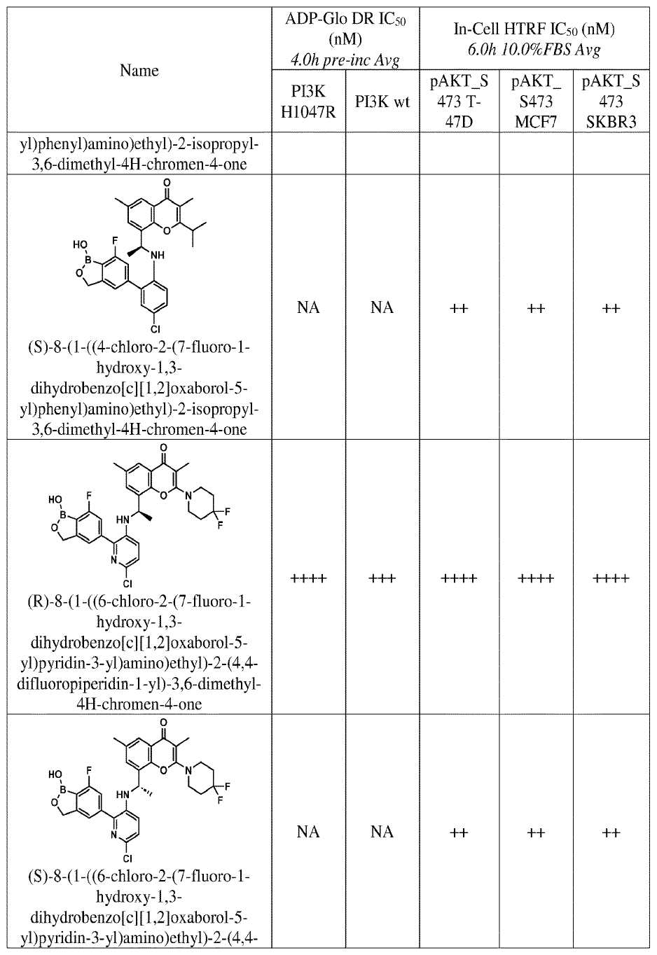

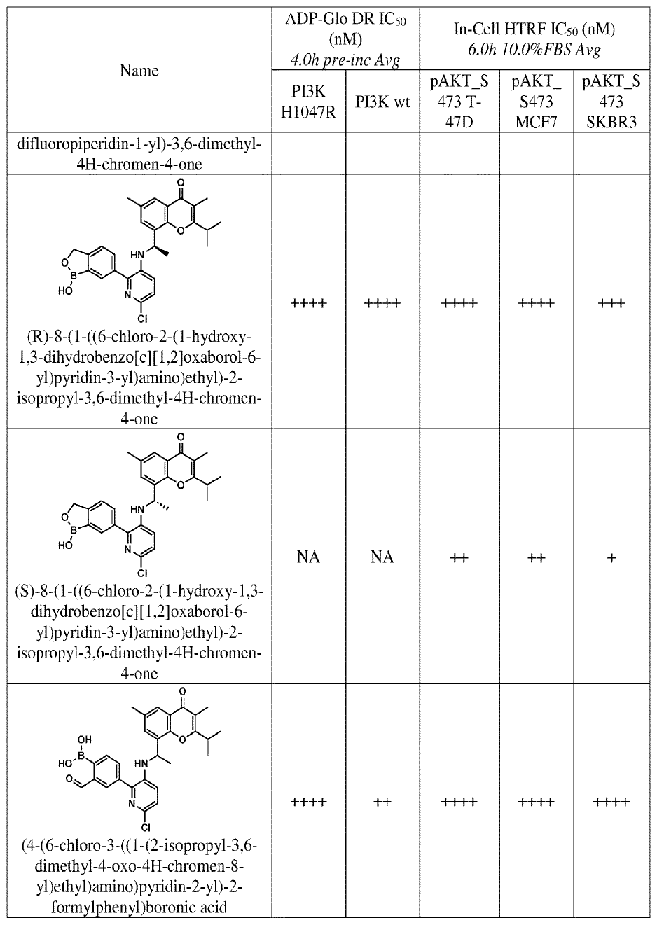

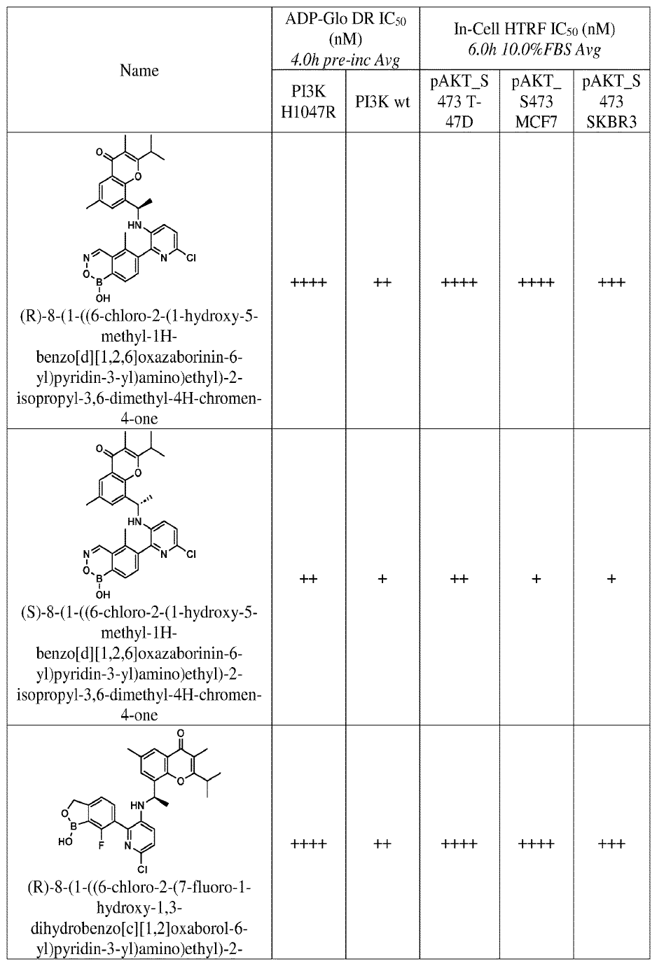

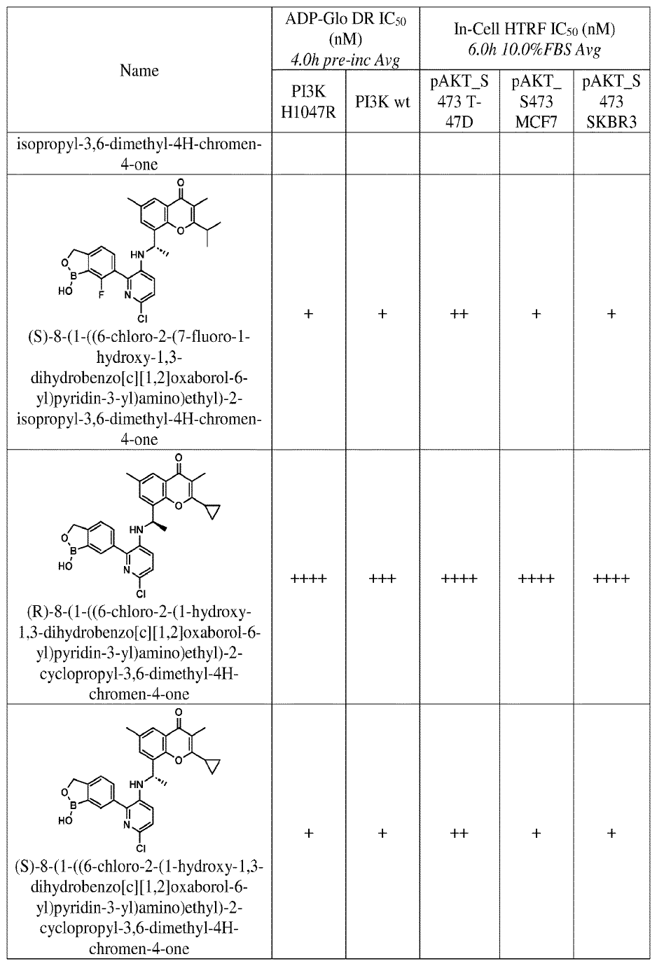

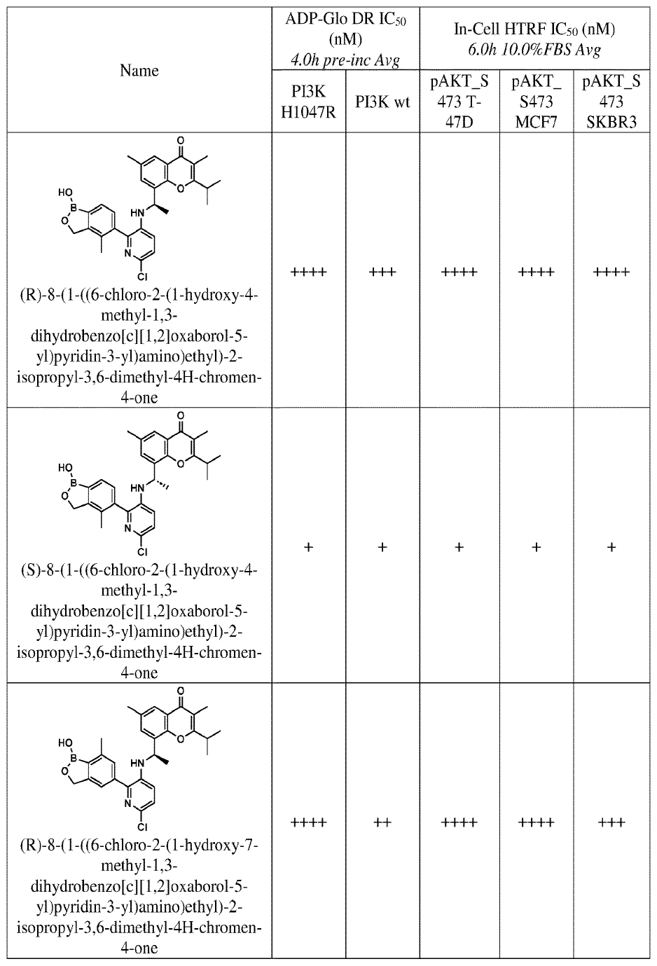

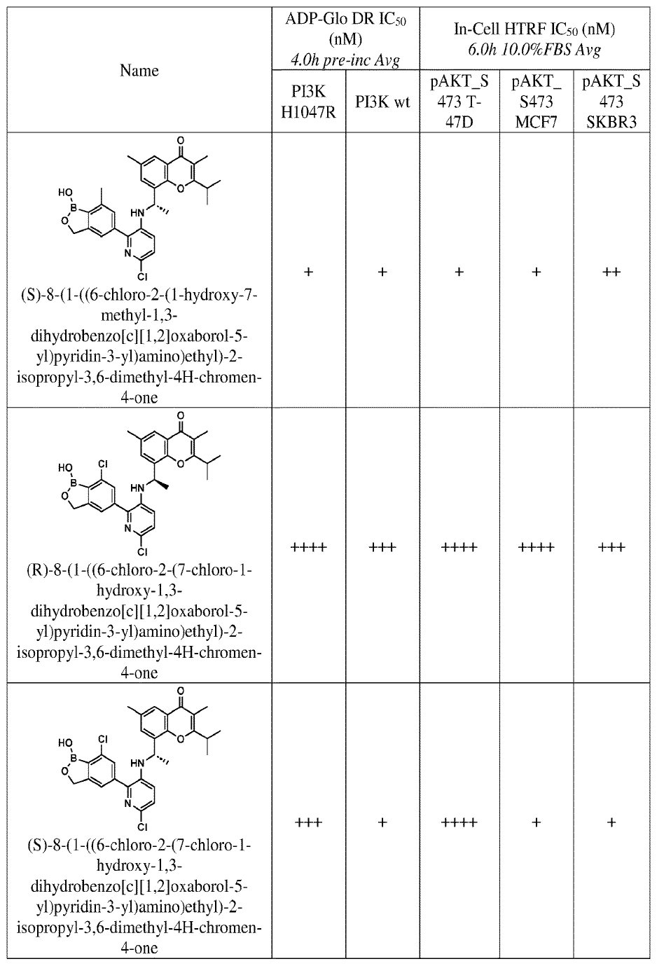

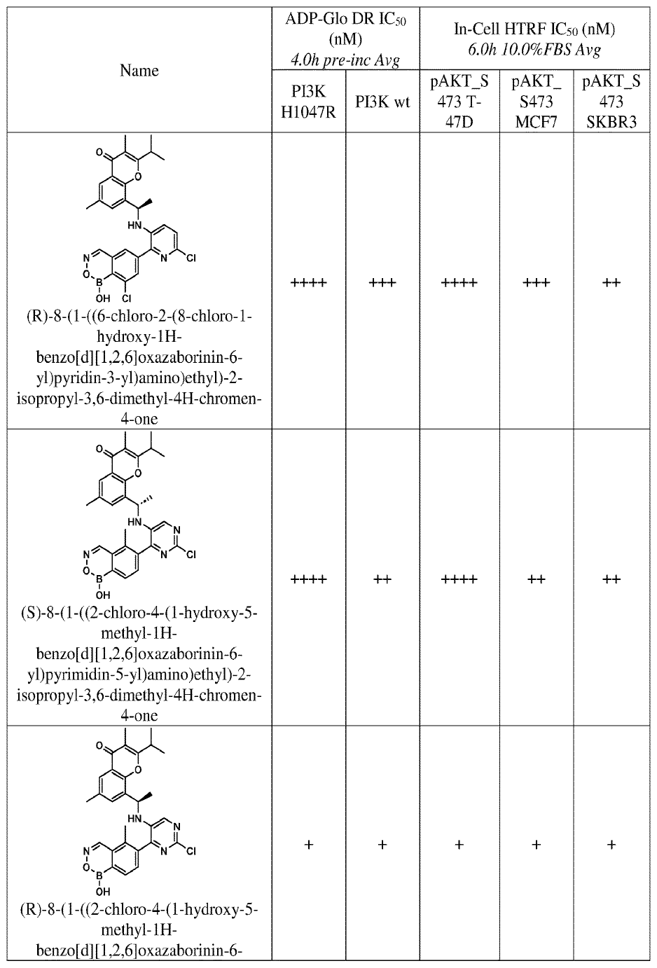

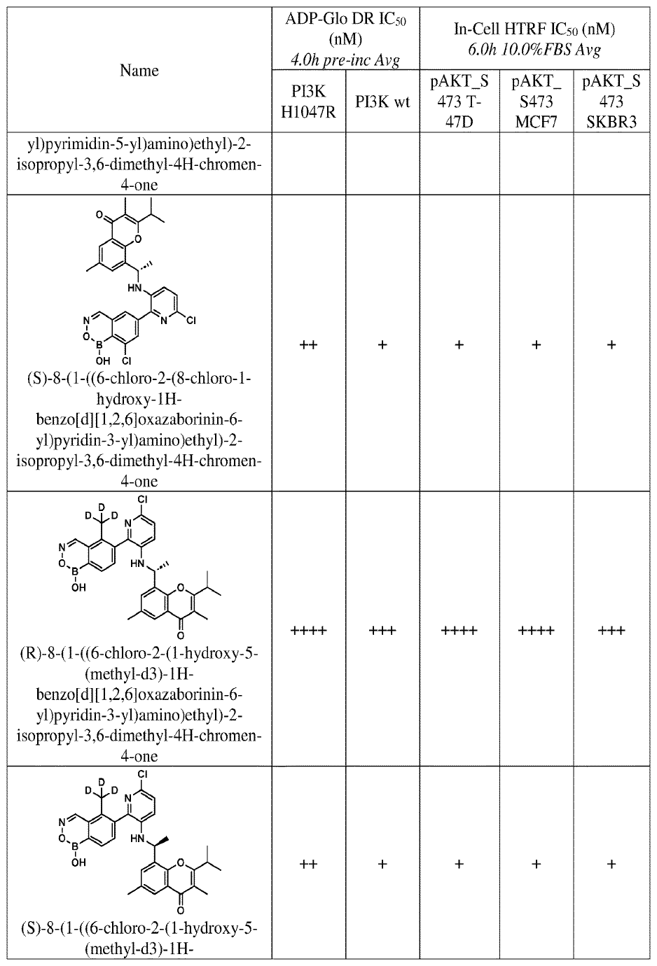

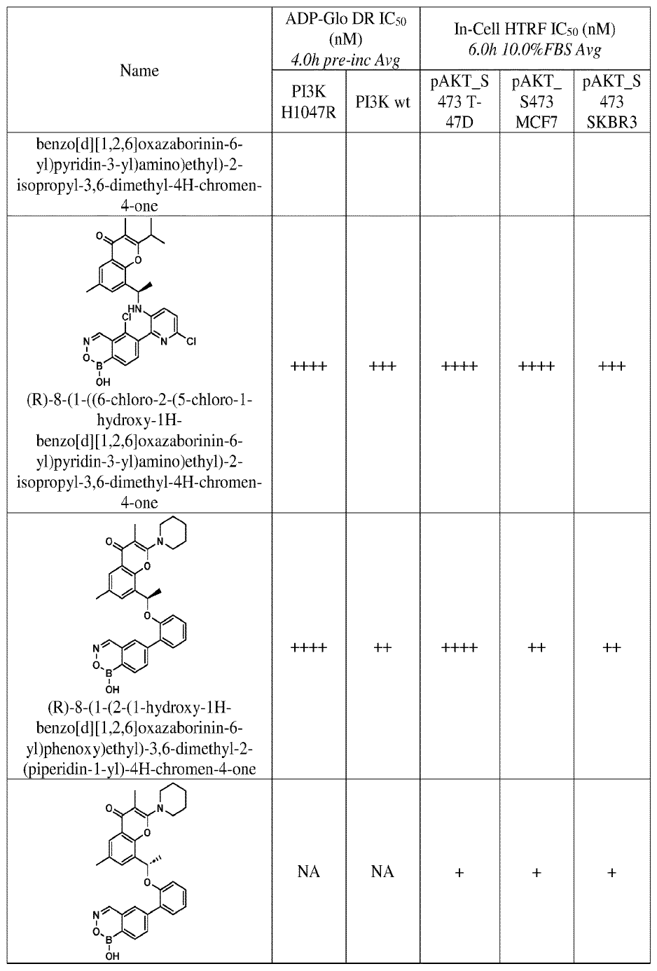

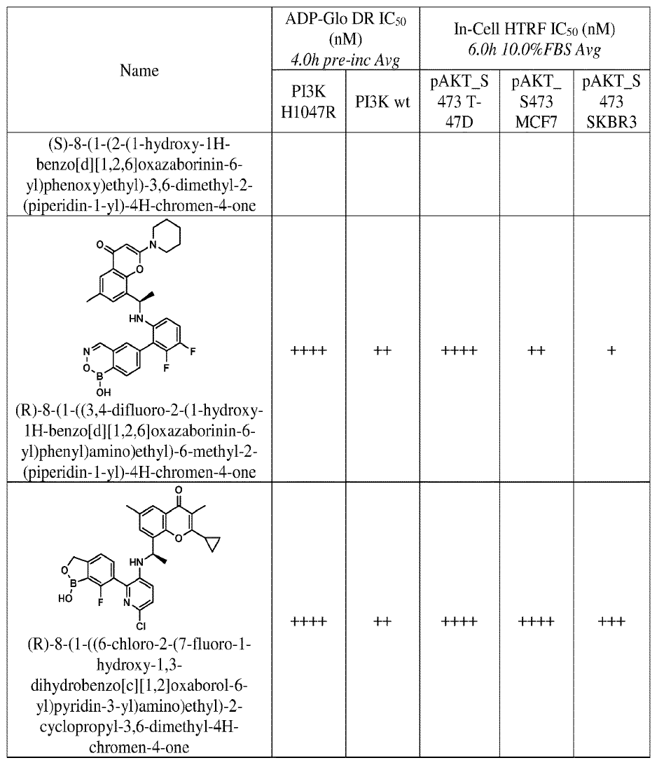

FIG. 1 provides tabulated biological data for compounds of the present disclosure according to the testing protocols described herein. One embodiment of the present disclosure includes all exemplified compounds. One embodiment of the present disclosure includes all compounds reported ++++, +++, ++, and +, in one or more assay. One embodiment of the present disclosure includes all compounds reported+++, ++, and +, in one or more assay. One embodiment of the present disclosure includes all compounds reported++ and +, in one or more assay. One embodiment of the present disclosure includes all compounds reported+, in one or more assay.

All publications, patents, and patent applications cited in this specification are incorporated herein by reference for the teaching to which such citation is used.

Test compounds for the experiments described herein were employed in free or salt form.

The specific responses observed may vary according to and depending on the particular active compound selected or whether there are present carriers, as well as the type of formulation and mode of administration employed, and such expected variations or differences in the results are contemplated in accordance with practice of the present disclosure.

Although specific embodiments of the present disclosure are herein illustrated and described in detail, the invention is not limited thereto. The above detailed descriptions are provided as exemplary of the present disclosure and should not be construed as constituting any limitation of the invention. Modifications will be obvious to those skilled in the art, and all modifications that do not depart from the spirit of the invention are intended to be included with the scope of the appended claims.

1. A compound of Formula (A): 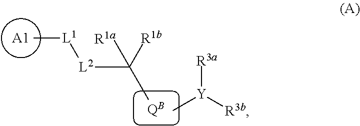
or a tautomer, enantiomer, diastereomer, mixture, salt, hydrate, solvate, or deuterated form thereof,
wherein
A1 is selected from the group consisting of: 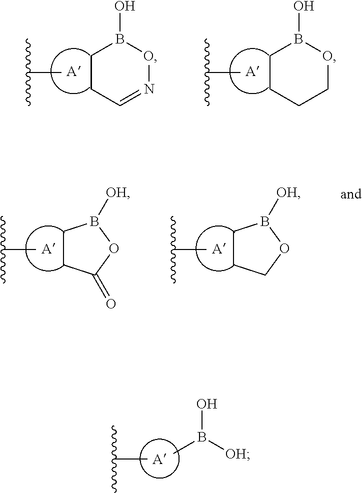
wherein each [symbol] represents the point of attachment to the depicted L¹;
each depicted A′ independently is selected from cycloalkyl, heterocyclyl, aryl, and heteroaryl;
wherein each of heterocyclyl and heteroaryl may contain one or more heteroatoms selected from N, O, and S;
each A1 is optionally substituted;
L¹ is selected from the group consisting of (i) a direct bond, (ii) optionally substituted arylene, and (iii) optionally substituted heteroarylene;
L² is selected from the group consisting of O and NH;
each of R^{1a} and R^{1b} independently is selected from the group consisting of H, halogen, CN, C₁₋₆ alkyl, C₂₋₆ alkenyl, C₂₋₆ alkynyl, C₁₋₆ haloalkyl, and C₁₋₆ alkoxy;
Q^{B} is an optionally substituted 8- to 14-membered fused ring system optionally containing one or more heteroatoms selected from the group consisting of O, N, and S; and
Y is H, CR^{3c}, N, O, or S; wherein
a) when Y is H, each of R^{3a} and R^{3b} is absent;
b) when Y is N,
each of R^{3a} and R^{3b} independently is selected from the group consisting of H, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl; or
Y, as an N atom, combines with R^{3a} and R^{3b} to form a heterocyclic or heteroaromatic mono ring or spiro or fused ring system, which may contain one or more additional heteroatoms selected from the group consisting of N, O, and S, and wherein the ring or ring system is optionally substituted;
c) when Y is CR^{3c},
R^{3c} is absent or is selected from the group consisting of hydrogen, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl;
each of R^{3a} and R^{3b} independently is selected from the group consisting of H, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl; or
Y, as a C atom, combines with R^{3a} and R^{3b} to form a cycloalkyl, aryl, heterocyclic, or heteroaromatic mono ring or spiro or fused ring system, where the heterocyclic and heteroaromatic rings contain one or more heteroatoms selected from the group consisting of N, O, and S, and wherein the Y-containing ring or ring system is optionally substituted; or
d) when Y is O or S, R^{3a} is absent and R^{3b} is selected from the group consisting of H, C₁₋₆ alkyl, C₂₋₆ alkenyl, and C₂₋₆ alkynyl.

2-11. (canceled)

12. The compound of claim 1, wherein Q^{B} is selected from the group consisting of: 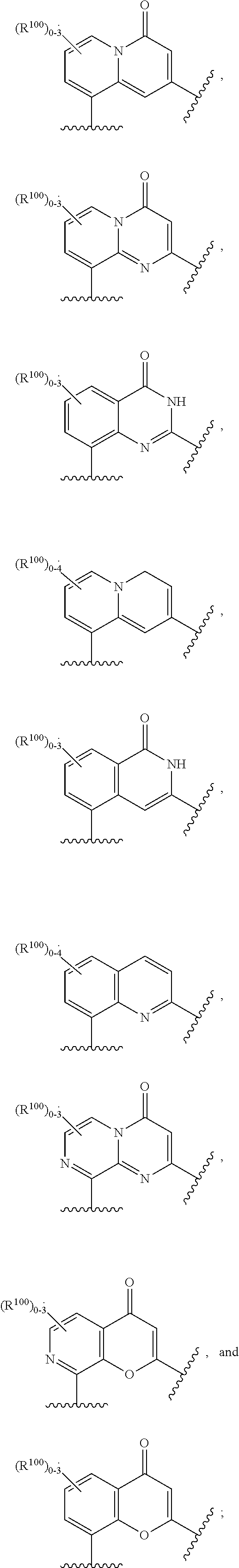
wherein
when present, each R¹⁰⁰ independently is selected from the group consisting of halogen, OH, oxo, C₁₋₆ alkyl, C₂₋₆ alkenyl, C₂₋₆ alkynyl, C₁₋₆ haloalkyl, C₁₋₆ alkoxy, (CH₂)_{q}—N(H or C₁₋₆alkyl), (CH₂)_{q}—O—C(O)—(CH₂)ᵣ—R¹⁴⁰, (CH₂)_{q}—NH—C(O)—(CH₂)ᵣ—R¹⁴⁰, (CH₂)_{q}—O—C(O)—(CH₂)ᵣ—OR¹⁴⁰, (CH₂)_{q}—NH—C(O)—(CH₂)ᵣ—OR¹⁴⁰, (CH₂)_{q}—O—(CH₂)ᵣ—R¹⁴⁰, (CH₂)_{q}—NH—(CH₂)ᵣ—R¹⁴⁰, (CH₂)_{q}—O—(CH₂)ᵣ—OR¹⁴⁰, (CH₂)_{q}—NH—(CH₂)ᵣ—OR¹⁴⁰, C₃₋₁₀ cycloalkyl, (CH₂)_{q}-heterocycle, (CH₂)_{q}-aryl, and (CH₂)_{q}-heteroaryl, wherein each of the cycloalkyl, heterocycle, aryl, and heteroaryl is optionally substituted with one or more halogen, OH, C₁₋₆ alkyl, C₁₋₆ haloalkyl, C₁₋₆ alkoxy, and C₁₋₆ haloalkoxy:
R¹⁴⁰ is an E3 ligase binding ligand; and
each of q and r, independently is, at each occurrence, selected from 0, 1, 2, 3, 4, 5, and 6.

13. The compound of claim 1, wherein Q^{B} is: 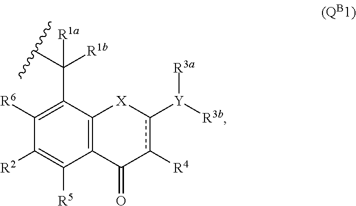
wherein
X is C(R^{x})₂, O, NR^{x}, or S;
each R^{x} independently is selected from the group consisting of H, C₁₋₆ alkyl, C₂₋₆ alkenyl, and C₂₋₆ alkynyl;
R² is selected from the group consisting of H, halogen, CN, C₁₋₆ alkyl, C₂₋₆ alkenyl, C₂₋₆ alkynyl, C₁₋₆ haloalkyl, C₂₋₆ haloalkenyl, C₂₋₆ haloalkynyl, C₁₋₆ alkoxy, (CH₂)₀₋₆—R^{QB1}, (CH₂)₀₋₆—OR^{QB1}, (CH₂)₀₋₆—N(R^{QB1})₂, (CH₂)₀₋₆—C(O)R^{QB1}, (CH₂)₀₋₆—C(O)OR^{QB1}, (CH₂)₀₋₆—C(O)N(R^{QB1})₂, (CH₂)₀₋₆—C₃₋₁₀ cycloalkyl, (CH₂)₀₋₆-aryl, (CH₂)₀₋₆-heterocycle, and (CH₂)₀₋₆-heteroaryl;
R⁴ is selected from the group consisting of H, halogen, CN, C₁₋₆ alkyl, C₂₋₆ alkenyl, C₂₋₆ alkynyl, C₁₋₆ haloalkyl, C₂₋₆ haloalkenyl, C₂₋₆ haloalkynyl, C₁₋₆ alkoxy, (CH₂)₀₋₆—R^{QB1}, (CH₂)₀₋₆—OR^{QB1}, (CH₂)₀₋₆—N(R^{QB1})₂, (CH₂)₀₋₆—C(O)R^{QB1}, (CH₂)₀₋₆—C(O)OR^{QB1}, (CH₂)₀₋₆—C(O)N(R^{QB1})₂, (CH₂)₀₋₆—C₃₋₁₀ cycloalkyl, (CH₂)₀₋₆-aryl, (CH₂)₀₋₆-heterocycle, and (CH₂)₀₋₆-heteroaryl;
R⁵ is selected from the group consisting of H, halogen, CN, C₁₋₆ alkyl, C₂₋₆ alkenyl, C₂₋₆ alkynyl, C₁₋₆ haloalkyl, C₂₋₆ haloalkenyl, C₂₋₆ haloalkynyl, C₁₋₆ alkoxy, (CH₂)₀₋₆—R^{QB1}, (CH₂)₀₋₆—OR^{QB1}, (CH₂)₀₋₆—N(R^{QB1})₂, (CH₂)₀₋₆—C(O)R^{QB1}, (CH₂)₀₋₆—C(O)OR^{QB1}, (CH₂)₀₋₆—C(O)N(R^{QB1})₂, (CH₂)₀₋₆—C₃₋₁₀ cycloalkyl, (CH₂)₀₋₆-aryl, (CH₂)₀₋₆-heterocycle, and (CH₂)₀₋₆-heteroaryl;
R⁶ is selected from the group consisting of H, halogen, CN, C₁₋₆ alkyl, C₂₋₆ alkenyl, C₂₋₆ alkynyl, C₁₋₆ haloalkyl, C₂₋₆ haloalkenyl, C₂₋₆ haloalkynyl, C₁₋₆ alkoxy, (CH₂)₀₋₆—R^{QB1}, (CH₂)₀₋₆—OR^{QB1}, (CH₂)₀₋₆—N(R^{QB1})₂, (CH₂)₀₋₆—C(O)R^{QB1}, (CH₂)₀₋₆—C(O)OR^{QB1}, (CH₂)₀₋₆—C(O)N(R^{QB1})₂, (CH₂)₀₋₆—C₃₋₁₀ cycloalkyl, (CH₂)₀₋₆-aryl, (CH₂)₀₋₆-heterocycle, and (CH₂)₀₋₆-heteroaryl; and
each R^{QB1} is independently H, halogen, OH, C₁₋₆ alkyl, C₂₋₆ alkenyl, C₂₋₆ alkynyl, C₁₋₆ haloalkyl, C₂₋₆ haloalkenyl, C₂₋₆ haloalkynyl, C₁₋₆ alkoxy, C₂₋₆ alkenoxy, C₂₋₆ alkynoxy, C₁₋₆ haloalkoxy, C₂₋₆ haloalkenoxy, or C₂₋₆ alkynoxy.

14. The compound of claim 13, wherein the compound is a compound of Formula A(Ia) or A(Ib): 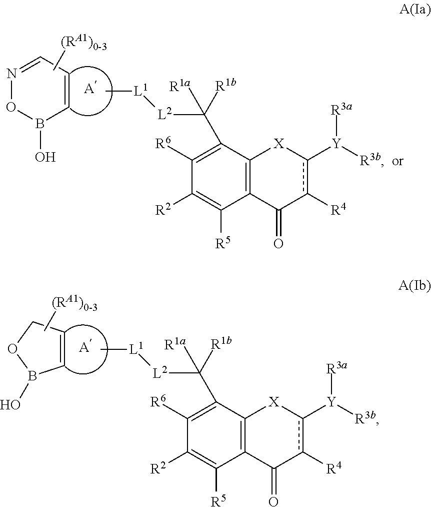
wherein,
each R^{A1} independently is selected from halogen, OH, C₁₋₆ alkyl, C₂₋₆ alkenyl, C₂₋₆ alkynyl, C₁₋₆ haloalkyl, C₂₋₆ haloalkenyl, C₂₋₆ haloalkynyl, (CH₂)₀₋₆CN, (CH₂)₁₋₆OH, (CH₂)₀₋₆O—C₁₋₆ alkyl, (CH₂)₀₋₆O—C₂₋₆ alkenyl, (CH₂)₀₋₆O—C₂₋₆ alkynyl, (CH₂)₀₋₆C(O)H, (CH₂)₀₋₆C(O)(C₁₋₆ alkyl, C₂₋₆ alkenyl, or C₂₋₆ alkynyl), (CH₂)₀₋₆C(O)OH, (CH₂)₀₋₆C(O)O(C₁₋₆ alkyl, C₂₋₆ alkenyl, or C₂₋₆ alkynyl), (CH₂)₀₋₆NH₂, (CH₂)₀₋₆NH(C₁₋₆ alkyl, C₂₋₆ alkenyl, or C₂₋₆ alkynyl), (CH₂)₀₋₆N(C₁₋₆ alkyl, C₂₋₆ alkenyl, or C₂₋₆ alkynyl)₂, (CH₂)₀₋₆C(O)NH₂, (CH₂)₀₋₆C(O)NH(C₁₋₆ alkyl, C₂₋₆ alkenyl, or C₂₋₆ alkynyl), (CH₂)₀₋₆C(O)N(C₁₋₆ alkyl, C₂₋₆ alkenyl, or C₂₋₆ alkynyl)₂, (CH₂)₀₋₆NHC(O)H, (CH₂)₀₋₆NHC(O)(C₁₋₆ alkyl, C₂₋₆ alkenyl, or C₂₋₆ alkynyl), and (CH₂)₀₋₆N(C₁₋₆ alkyl, C₂₋₆ alkenyl, and C₂₋₆ alkynyl)C(O)(C₁₋₆ alkyl, C₂₋₆ alkenyl, or C₂₋₆ alkynyl), or an isotopic form of R^{A1}.

15-37. (canceled)

38. A compound of Formula (AWH): 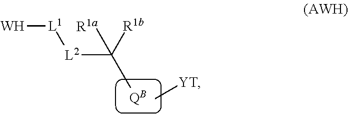
or a tautomer, enantiomer, diastereomer, mixture, salt, hydrate, solvate, or deuterated form thereof, wherein
WH is a warhead moiety;
L¹ is selected from the group consisting of a direct bond, optionally substituted cycloalkylene, optionally substituted heterocyclylene, optionally substituted arylene, and optionally substituted heteroarylene;
L² is selected from the group consisting of (CH₂)₁₋₆, O, C(O), S, and NH;
each of R^{1a} and R^{1b} independently is selected from the group consisting of H, halogen, CN, C₁₋₆ alkyl, C₂₋₆ alkenyl, C₂₋₆ alkynyl, C₁₋₆ haloalkyl, and C₁₋₆ alkoxy;
or L²-C(R^{1a})(R^{1b}) combines to form NHC(S), C(S)NH, NHC(O), C(O)NH, NHS(O)₂, S(O)₂NH, NHC(NH), or C(NH)NH;
Q^{B} is an optionally substituted 8- to 14-membered spiro or fused ring system optionally containing one or more heteroatoms selected from the group consisting of O, N, and S;
YT is any moiety that provides desired physicochemical properties; and
an optional E3 ligase binding ligand.

39-41. (canceled)

42. The compound of claim 38, wherein WH is selected from: 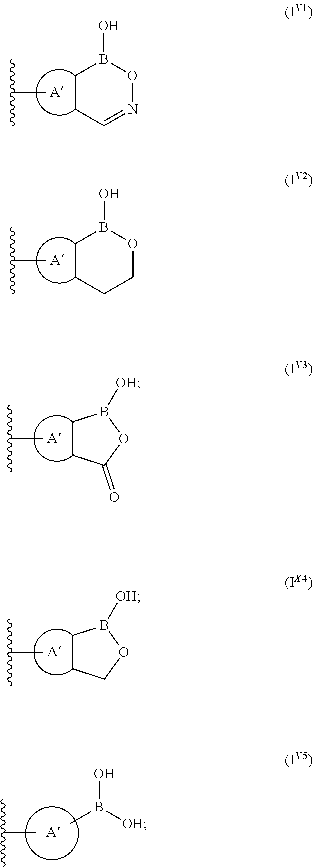
wherein each [symbol] represents the point of attachment to the depicted L¹;
each depicted A′ is a cycloalkyl, heterocyclyl, aryl, or heteroaryl mono-ring;
wherein each of the heterocyclyl or heteroaryl rings contain one or more heteroatoms selected from N, O, and S; and
each is optionally substituted with one or more R^{A1}, wherein each R^{A1} independently is selected from halogen, OH, C₁₋₆ alkyl, C₂₋₆ alkenyl, C₂₋₆ alkynyl, C₁₋₆ haloalkyl, C₂₋₆ haloalkenyl, C₂₋₆ haloalkynyl, (CH₂)₀₋₆CN, (CH₂)₁₋₆OH, (CH₂)₀₋₆O—C₁₋₆ alkyl, (CH₂)₀₋₆O—C₂₋₆ alkenyl, (CH₂)₀₋₆O—C₂₋₆ alkynyl, (CH₂)₀₋₆C(O)H, (CH₂)₀₋₆C(O)(C₁₋₆ alkyl, C₂₋₆ alkenyl, or C₂₋₆ alkynyl), (CH₂)₀₋₆C(O)OH, (CH₂)₀₋₆C(O)O(C₁₋₆ alkyl, C₂₋₆ alkenyl, or C₂₋₆ alkynyl), (CH₂)₀₋₆NH₂, (CH₂)₀₋₆NH(C₁₋₆ alkyl, C₂₋₆ alkenyl, or C₂₋₆ alkynyl), (CH₂)₀₋₆N(C₁₋₆ alkyl, C₂₋₆ alkenyl, or C₂₋₆ alkynyl)₂, (CH₂)₀₋₆C(O)NH₂, (CH₂)₀₋₆C(O)NH(C₁₋₆ alkyl, C₂₋₆ alkenyl, or C₂₋₆ alkynyl), (CH₂)₀₋₆C(O)N(C₁₋₆ alkyl, C₂₋₆ alkenyl, or C₂₋₆ alkynyl)₂, (CH₂)₀₋₆NHC(O)H, (CH₂)₀₋₆NHC(O)(C₁₋₆ alkyl, C₂₋₆ alkenyl, or C₂₋₆ alkynyl), and (CH₂)₀₋₆N(C₁₋₆ alkyl, C₂₋₆ alkenyl, and C₂-6 alkynyl)C(O)(C₁₋₆ alkyl, C₂₋₆ alkenyl, or C₂₋₆ alkynyl), or an isotopic form of R^{A1}.

43-49. (canceled)

50. The compound of claim 38, wherein Q^{B} is selected from: 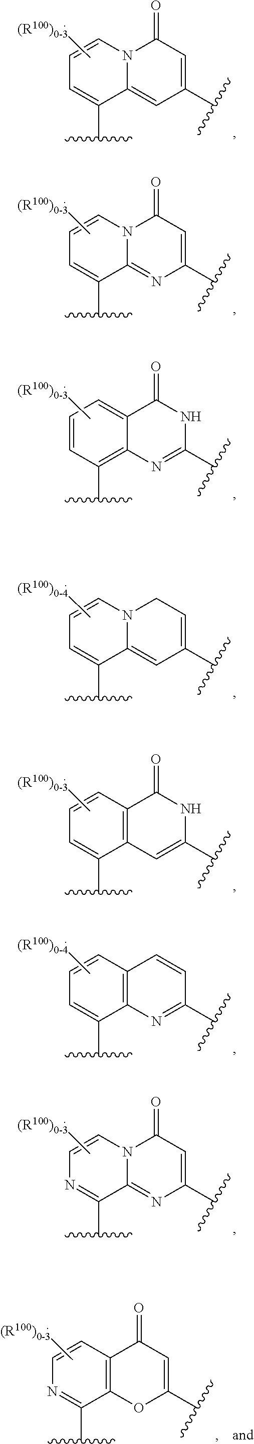 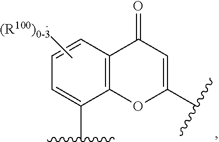
wherein
each R¹⁰⁰ independently is selected from the group consisting of halogen, OH, oxo, C₁₋₆ alkyl, C₂₋₆ alkenyl, C₂₋₆ alkynyl, C₁₋₆ haloalkyl, C₂₋₆ haloalkenyl, C₂₋₆ haloalkynyl, C₁₋₆ alkoxy, (CH₂)_{q}—N(H or C₁₋₆alkyl), (CH₂)_{q}—O—C(O)—(CH₂)ᵣ—R¹⁴⁰, (CH₂)_{q}—NH—C(O)—(CH₂)ᵣ—R¹⁴⁰, (CH₂)_{q}—O—C(O)—(CH₂)ᵣ—OR¹⁴⁰, (CH₂)—NH—C(O)—(CH₂)ᵣ—OR¹⁴⁰, (CH₂)—O(CH₂)ᵣ—R¹⁴⁰, (CH₂)—NH—(CH₂)ᵣ—R⁴⁰, (CH₂)—O—(CH₂)ᵣ—OR¹⁴⁰, (CH₂)_{q}—NH—(CH₂)ᵣ—OR¹⁴⁰, C₃₋₁₀ cycloalkyl, (CH₂)_{q}-heterocycle, (CH₂)_{q}-aryl, and (CH₂)_{q}-heteroaryl, wherein each of the cycloalkyl, heterocycle, aryl, and heteroaryl is optionally substituted with one or more halogen, OH, C₁₋₆ alkyl, C₁₋₆ haloalkyl, C₁-6 alkoxy, and C₁₋₆ haloalkoxy;
R¹⁴⁰ is an E3 ligase binding ligand; and
each of q and r, independently is, at each occurrence, selected from 0, 1, 2, 3, 4, 5, and 6.

51-53. (canceled)

54. The compound of claim 38, wherein the compound is a compound of (AWH2): 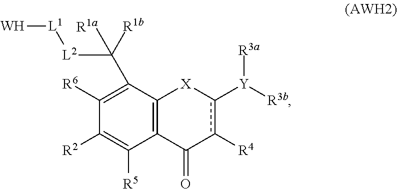
or a tautomer, enantiomer, diastereomer, mixture, salt, hydrate, solvate, or deuterated form thereof,
wherein
X is C(R^{x})₂, O, NR^{x}, or S;
each R independently is selected from the group consisting of H, C₁₋₆ alkyl, C₂₋₆ alkenyl, and C₂₋₆ alkynyl;
R² is selected from the group consisting of H, halogen, CN, C₁₋₆ alkyl, C₂₋₆ alkenyl, C₂₋₆ alkynyl, C₁₋₆ haloalkyl, C₁₋₆ alkoxy, (CH₂)₀₋₆—R^{AWH2}, (CH₂)₀₋₆—OR^{AWH2}, (CH₂)₀₋₆—N(R^{AWH2})₂, (CH₂)₀₋₆—C(O)R^{AWH2}, (CH₂)₀₋₆—C(O)OR^{AWH2}, (CH₂)₀₋₆—C(O)N(R^{AWH2})₂, (CH₂)₀₋₆—C₃₋₁₀ cycloalkyl, (CH₂)₀₋₆-aryl, (CH₂)₀₋₆-heterocycle, and (CH₂)₀₋₆-heteroaryl;
R⁴ is selected from the group consisting of H, halogen, CN, C₁₋₆ alkyl, C₂₋₆ alkenyl, C₂₋₆ alkynyl, C₁₋₆ haloalkyl, C₁₋₆ alkoxy, (CH₂)₀₋₆—R^{AWH2}, (CH₂)₀₋₆—OR^{AWH2}, (CH₂)₀₋₆—N(R^{AWH2})₂, (CH₂)₀₋₆—C(O)R^{AWH2}, (CH₂)₀₋₆—C(O)OR^{AWH2}, (CH₂)₀₋₆—C(O)N(R^{AWH2})₂, (CH₂)₀₋₆—C₃₋₁₀ cycloalkyl, (CH₂)₀₋₆-aryl, (CH₂)₀₋₆-heterocycle, and (CH₂)₀₋₆-heteroaryl;
R⁵ is selected from the group consisting of H, halogen, CN, C₁₋₆ alkyl, C₂₋₆ alkenyl, C₂₋₆ alkynyl, C₁₋₆ haloalkyl, C₁₋₆ alkoxy, (CH₂)₀₋₆—R^{AWH2}, (CH₂)₀₋₆—OR^{AWH2}, (CH₂)₀₋₆—N(R^{AWH2})₂, (CH₂)₀₋₆—C(O)R^{AWH2}, (CH₂)₀₋₆—C(O)OR^{AWH2}, (CH₂)₀₋₆—C(O)N(R^{AWH2})₂, (CH₂)₀₋₆—C₃₋₁₀ cycloalkyl, (CH₂)₀₋₆-aryl, (CH₂)₀₋₆-heterocycle, and (CH₂)₀₋₆-heteroaryl;
R⁶ is selected from the group consisting of H, halogen, CN, C₁₋₆ alkyl, C₂₋₆ alkenyl, C₂₋₆ alkynyl, C₁₋₆ haloalkyl, C₁₋₆ alkoxy, (CH₂)₀₋₆—R^{AWH2}, (CH₂)₀₋₆—OR^{AWH2}, (CH₂)₀₋₆—N(R^{AWH2})₂, (CH₂)₀₋₆—C(O)R^{AWH2}, (CH₂)₀₋₆—C(O)OR^{AWH2}, (CH₂)₀₋₆—C(O)N(R^{AWH2})₂, (CH₂)₀₋₆—C₃₋₁₀ cycloalkyl, (CH₂)₀₋₆-aryl, (CH₂)₀₋₆-heterocycle, and (CH₂)₀₋₆-heteroaryl; and
each R^{AWH2} is independently H, halogen, OH, C₁₋₆ alkyl, C₂₋₆ alkenyl, C₂₋₆ alkynyl, C₁₋₆ haloalkyl, C₂₋₆ haloalkenyl, C₂₋₆ haloalkynyl, C₁₋₆ alkoxy, C₂₋₆ alkenoxy, C₂₋₆ alkynoxy, C₁₋₆ haloalkoxy, C₂₋₆ haloalkenoxy, or C₂₋₆ alkynoxy.

55-68. (canceled)

69. A compound of Formula (AX): 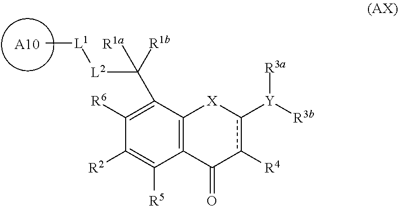
or a tautomer, enantiomer, diastereomer, mixture, salt, hydrate, solvate, or deuterated form thereof,
wherein
A10 is a 5- to 14-membered mono- or fused-ring system comprising one or more of cycloalkyl, heterocyclyl, aryl, and heteroaryl,
each of heterocyclyl or heteroaryl rings may contain one or more heteroatoms selected from N, O, S, and B;
A10 is optionally substituted with one or more R^{A10};
each R^{A10} independently is selected from halogen, OH, C₁₋₆ alkyl, C₂₋₆ alkenyl, C₂₋₆ alkynyl, C₁₋₆ haloalkyl, C₂₋₆ haloalkenyl, C₂₋₆ haloalkynyl, oxo, (CH₂)₀₋₆CN, (CH₂)₁₋₆OH, (CH₂)₀₋₆O—C₁₋₆ alkyl, (CH₂)₀₋₆O—C₂₋₆ alkenyl, (CH₂)₀₋₆O—C₂₋₆ alkynyl, (CH₂)₀₋₆C(O)H, (CH₂)₀₋₆C(O)(C₁₋₆ alkyl, C₂₋₆ alkenyl, or C₂₋₆ alkynyl), (CH₂)₀₋₆C(O)OH, (CH₂)₀₋₆C(O)O(C₁₋₆ alkyl, C₂₋₆ alkenyl, or C₂₋₆ alkynyl), (CH₂)₀₋₆NH₂, (CH₂)₀₋₆NH(C₁₋₆ alkyl, C₂₋₆ alkenyl, or C₂₋₆ alkynyl), (CH₂)₀₋₆N(C₁₋₆ alkyl, C₂₋₆ alkenyl, or C₂₋₆ alkynyl)₂, (CH₂)₀₋₆C(O)NH₂, (CH₂)₀₋₆C(O)NH(C₁₋₆ alkyl, C₂₋₆ alkenyl, or C₂₋₆ alkynyl), (CH₂)₀₋₆C(O)N(C₁₋₆ alkyl, C₂₋₆ alkenyl, or C₂₋₆ alkynyl)₂, (CH₂)₀₋₆NHC(O)H, (CH₂)₀₋₆NHC(O)(C₁₋₆ alkyl, C₂₋₆ alkenyl, or C₂₋₆ alkynyl), (CH₂)₀₋₆N(C₁₋₆ alkyl, C₂₋₆ alkenyl, and C₂₋₆ alkynyl)C(O)(C₁₋₆ alkyl, C₂₋₆ alkenyl, or C₂₋₆ alkynyl), B(OH)₂, SO₂-halogen, and O—SO₂-halogen;
L¹ is selected from the group consisting of a direct bond, optionally substituted arylene, and optionally substituted heteroarylene;
L² is selected from the group consisting of 0 and NH;
each of R^{1a} and R^{1b} independently is selected from the group consisting of H, halogen, CN, C₁₋₆ alkyl, C₂₋₆ alkenyl, C₂₋₆ alkynyl, C₁₋₆ haloalkyl, and C₁₋₆ alkoxy;
or L²-C(R^{1a})(R^{1b}) combines to form NHC(O), C(O)NH, NHS(O)₂, S(O)₂NH, or C(NH)NH₂;
X is C(R^{x})₂, O, NR^{x}, or S;
each R independently is selected from the group consisting of H, C₁₋₆ alkyl, C₂₋₆ alkenyl, and C₂₋₆ alkynyl;
Y is H, CR^{3c}, N, O, or S; wherein
a) when Y is H, each of R^{3a} and R^{3b} is absent;
b) when Y is N,
each of R^{3a} and R^{3b} independently is selected from the group consisting of H, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl; or
Y, as an N atom, combines with R^{3a} and R^{3b} to form a heterocyclic or heteroaromatic mono ring or spiro or fused ring system, which may contain one or more additional heteroatoms selected from the group consisting of N, O, and S, and wherein the ring or ring system is optionally substituted;
c) when Y is CR^{3c},
R^{3e} is absent or is selected from the group consisting of hydrogen, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl;
each of R^{3a} and R^{3b} independently is selected from the group consisting of H, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl; or
Y, as a C atom, combines with R^{3a} and R^{3b} to form a cycloalkyl, aryl, heterocyclic, or heteroaromatic mono ring or spiro or fused ring system, where the heterocyclic and heteroaromatic rings contain one or more heteroatoms selected from the group consisting of N, O, and S, and wherein the Y-containing ring or ring system is optionally substituted; or
d) when Y is O or S, R^{3a} is absent and R^{3b} is selected from the group consisting of H, C₁₋₆ alkyl, C₂₋₆ alkenyl, and C₂₋₆ alkynyl;
R² is selected from the group consisting of H, halogen, CN, C₁₋₆ alkyl, C₂₋₆ alkenyl, C₂₋₆ alkynyl, C₁₋₆ haloalkyl, C₂₋₆ haloalkenyl, C₂₋₆ haloalkynyl, C₁₋₆ alkoxy, (CH₂)₀₋₆—R^{AX}, (CH₂)₀₋₆—OR^{AX}, (CH₂)₀₋₆—N(R^{AX})₂, (CH₂)₀₋₆—C(O)R^{AX}, (CH₂)₀₋₆—C(O)OR^{AX}, (CH₂)₀₋₆—C(O)N(R^{AX})₂, (CH₂)₀₋₆—C₃₋₁₀ cycloalkyl, (CH₂)₀₋₆-aryl, (CH₂)₀₋₆-heterocycle, and (CH₂)₀₋₆-heteroaryl;
R⁴ is selected from the group consisting of H, halogen, CN, C₁₋₆ alkyl, C₂₋₆ alkenyl, C₂₋₆ alkynyl, C₁₋₆ haloalkyl, C₂₋₆ haloalkenyl, C₂₋₆ haloalkynyl, C₁₋₆ alkoxy, (CH₂)₀₋₆—R^{AX}, (CH₂)₀₋₆—OR^{AX}, (CH₂)₀₋₆—N(R^{AX})₂, (CH₂)₀₋₆—C(O)R^{AX}, (CH₂)₀₋₆—C(O)OR^{AX}, (CH₂)₀₋₆—C(O)N(R^{AX})₂, (CH₂)₀₋₆—C₃₋₁₀ cycloalkyl, (CH₂)₀₋₆-aryl, (CH₂)₀₋₆-heterocycle, and (CH₂)₀₋₆-heteroaryl;
R⁵ is selected from the group consisting of H, halogen, CN, C₁₋₆ alkyl, C₂₋₆ alkenyl, C₂₋₆ alkynyl, C₁₋₆ haloalkyl, C₂₋₆ haloalkenyl, C₂₋₆ haloalkynyl, C₁₋₆ alkoxy, (CH₂)₀₋₆—R^{AX}, (CH₂)₀₋₆—OR^{AX}, (CH₂)₀₋₆—N(R^{AX})₂, (CH₂)₀₋₆—C(O)R^{AX}, (CH₂)₀₋₆—C(O)OR^{AX}, (CH₂)₀₋₆—C(O)N(R^{AX})₂, (CH₂)₀₋₆—C₃₋₁₀ cycloalkyl, (CH₂)₀₋₆-aryl, (CH₂)₀₋₆-heterocycle, and (CH₂)₀₋₆-heteroaryl;
R⁶ is selected from the group consisting of H, halogen, CN, C₁₋₆ alkyl, C₂₋₆ alkenyl, C₂₋₆ alkynyl, C₁₋₆ haloalkyl, C₂₋₆ haloalkenyl, C₂₋₆ haloalkynyl, C₁₋₆ alkoxy, (CH₂)₀₋₆—R^{AX}, (CH₂)₀₋₆—OR^{AX}, (CH₂)₀₋₆—N(R^{AX})₂, (CH₂)₀₋₆—C(O)R^{AX}, (CH₂)₀₋₆—C(O)OR^{AX}, (CH₂)₀₋₆—C(O)N(R^{AX})₂, (CH₂)₀₋₆—C₃₋₁₀ cycloalkyl, (CH₂)₀₋₆-aryl, (CH₂)₀₋₆-heterocycle, and (CH₂)₀₋₆-heteroaryl; and
each R^{AX} is independently H, halogen, OH, C₁₋₆ alkyl, C₂₋₆ alkenyl, C₂₋₆ alkynyl, C₁₋₆ haloalkyl, C₂₋₆ haloalkenyl, C₂₋₆ haloalkynyl, C₁₋₆ alkoxy, C₂₋₆ alkenoxy, C₂₋₆ alkynoxy, C₁₋₆ haloalkoxy, C₂₋₆ haloalkenoxy, or C₂₋₆ alkynoxy.

70. (canceled)

71. The compound of claim 69, wherein A10 is selected from the group consisting of: 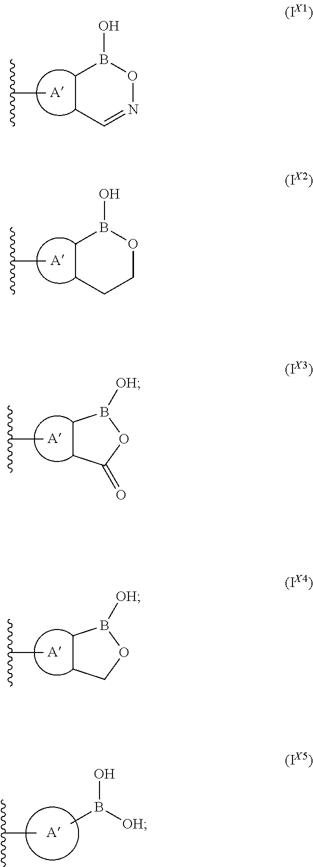
wherein each [symbol] represents the point of attachment to the depicted L¹;
each depicted A′ independently is a cycloalkyl, heterocyclyl, aryl, or heteroaryl; and
each A10 is optionally substituted with one or more R^{A10} on either depicted ring.

72.-95. (canceled)

96. A compound of Formula Ia or Ib: 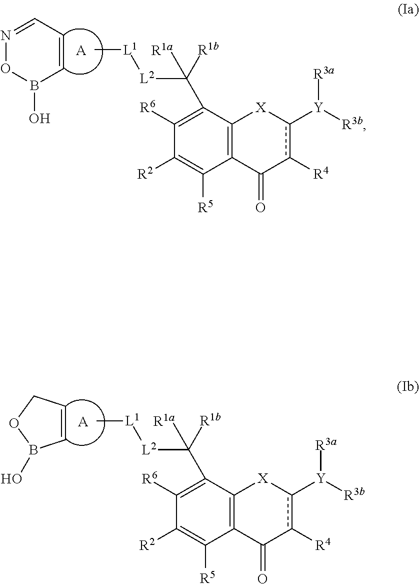
or a tautomer, enantiomer, diastereomer, mixture, salt, hydrate, solvate, or deuterated form thereof, wherein
A is an aryl or heteroaryl ring, and together with the depicted boron ring, creates a fused ring system that includes the depicted boron-containing ring, and which system is optionally substituted with one or more R^{A};
each R^{A} independently is selected from halogen, OH, C₁₋₆ alkyl, C₂₋₆ alkenyl, C₂₋₆ alkynyl, C₁₋₆ haloalkyl, C₂₋₆ haloalkenyl, C₂₋₆ haloalkynyl, (CH₂)₀₋₆CN, (CH₂)₁₋₆OH, (CH₂)₀₋₆O—C₁₋₆ alkyl, (CH₂)₀₋₆O—C₂₋₆ alkenyl, (CH₂)₀₋₆O—C₂₋₆ alkynyl, (CH₂)₀₋₆C(O)H, (CH₂)₀₋₆C(O)(C₁₋₆ alkyl, C₂₋₆ alkenyl, or C₂₋₆ alkynyl), (CH₂)₀₋₆C(O)OH, (CH₂)₀₋₆C(O)₀₋₆ (C₁₋₆ alkyl, C₂₋₆ alkenyl, or C₂₋₆ alkynyl), (CH₂)₀₋₆NH₂, (CH₂)₀₋₆NH(C₁₋₆ alkyl, C₂₋₆ alkenyl, or C₂₋₆ alkynyl), (CH₂)₀₋₆N(C₁₋₆ alkyl, C₂₋₆ alkenyl, or C₂₋₆ alkynyl)₂, (CH₂)₀₋₆C(O)NH₂, (CH₂)₀₋₆C(O)NH(C₁₋₆ alkyl, C₂₋₆ alkenyl, or C₂₋₆ alkynyl), (CH₂)₀₋₆C(O)N(C₁₋₆ alkyl, C₂₋₆ alkenyl, or C₂₋₆ alkynyl)₂, (CH₂)₀₋₆NHC(O)H, (CH₂)₀₋₆NHC(O)(C₁₋₆ alkyl, C₂₋₆ alkenyl, or C₂₋₆ alkynyl), and (CH₂)₀₋₆N(C₁₋₆ alkyl, C₂₋₆ alkenyl, and C₂₋₆ alkynyl)C(O)(C₁₋₆ alkyl, C₂₋₆ alkenyl, or C₂₋₆ alkynyl) or a deuterated form thereof,
L¹ is selected from the group consisting of a direct bond, optionally substituted arylene, and optionally substituted heteroarylene;
L² is selected from the group consisting of O and NH;
each of R^{1a} and R^{1b} independently is selected from the group consisting of H, halogen, CN, C₁₋₆ alkyl, C₂₋₆ alkenyl, C₂₋₆ alkynyl, C₁₋₆ haloalkyl, and C₁₋₆ alkoxy;
X is C(R^{x})₂, O, NR^{x}, or S;
each R independently is selected from the group consisting of H, C₁₋₆ alkyl, C₂₋₆ alkenyl, and C₂₋₆ alkynyl;
each of R², R⁴, R⁵, and R⁶ independently is H, halogen, C₁₋₆ alkyl, C₂₋₆ alkenyl, C₂₋₆ alkynyl, C₃-C₆ cycloalkyl, C₂₋₆ haloalkenyl, C₂₋₆ haloalkynyl, or C₁₋₆ haloalkyl; and
Y is N or CR^{3a}; wherein
a) when Y is N,
each of R^{3a} and R^{3b} independently is selected from the group consisting of H, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl; or
R^{3a} and R^{3b} combine with the N atom to form a heterocyclic or heteroaromatic mono ring or spiro or fused ring system, which may contain one or more additional heteroatoms selected from the group consisting of N, O, and S, and wherein the ring or ring system is optionally substituted;
b) when Y is CR^{3c},
R^{3c} is absent or is selected from the group consisting of hydrogen, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl;
each of R^{3a} and R^{3b} independently is selected from the group consisting of H, C₁₋₆ alkyl, C₂₋₆ alkenyl, C₂₋₆ alkynyl, C₁₋₆ haloalkyl, C₂₋₆ haloalkenyl, C₂₋₆ haloalkynyl, aryl, C₃₋₆ cycloalkyl, and 3 to 6 membered heterocyclyl; or
R^{3a} and R^{3b} combine with the C atom to form a cycloalkyl, aryl, heterocyclic, or heteroaromatic mono ring or spiro or fused ring system, where the heterocyclic and heteroaromatic rings contain one or more heteroatoms selected from the group consisting of N, O, and S, and wherein the Y-containing ring or ring system is optionally substituted.

97.-126. (canceled)

127. A compound of Formula Xa or Xb: 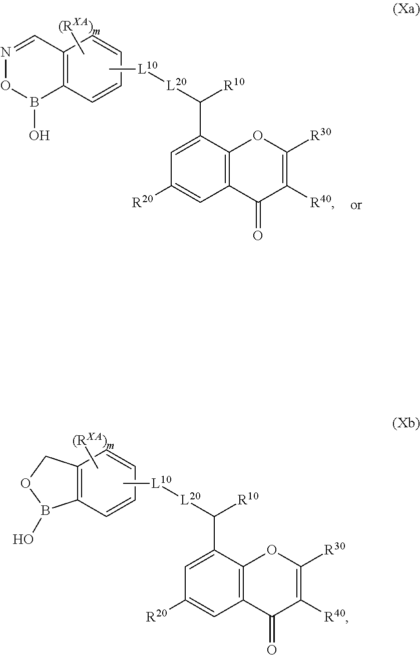
or a tautomer, enantiomer, diastereomer, mixture, salt, hydrate, solvate, or deuterated form thereof, wherein
each m is independently 0, 1, 2, 3, 4, or 5;
each R^{XA} independently is selected from halogen, C₁₋₆ alkyl, C₂₋₆ alkenyl, C₂₋₆ alkynyl, C₁₋₆ haloalkyl, C₁₋₆ alkoxy, and C₃₋₆ cycloalkyl, or a deuterated form thereof,
L¹⁰ is selected from the group consisting of a direct bond, optionally substituted arylene, or optionally substituted heteroarylene;
L²⁰ is selected from the group consisting of O and NH;
R¹⁰ is selected from the group consisting of H and C₁₋₆ alkyl;
R²⁰ is selected from the group consisting of H, halogen, C₁-C₆ haloalkyl, and C₁₋₆ alkyl;
R³⁰ is selected from the group consisting of H or optionally substituted C₁₋₆ alkyl, C₂₋₆ alkenyl, C₂₋₆ alkynyl, C₃₋₆ cycloalkyl, N(C₁-C₆ alkyl), 6-membered aryl, 5- or 6-membered heteroaryl, or 3- to 10-membered heterocyclyl; and
R⁴⁰ is selected from the group consisting of H and C₁₋₆ alkyl.

128-150. (canceled)

151. A compound selected from the group consisting of: 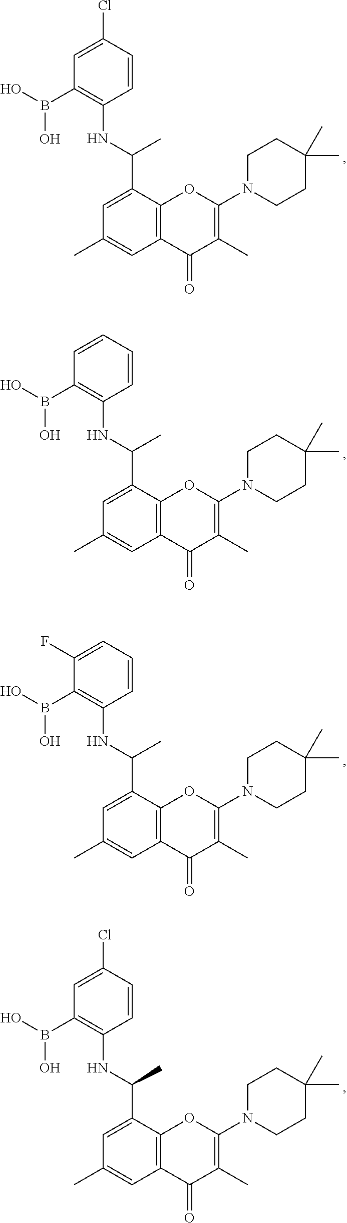 [SMILES: Cc1cc(C(C)Nc2ccc(Cl)cc2B(O)O)c2oc(N3CCC(C)(C)CC3)c(C)c(=O)c2c1 ; Cc1cc(C(C)Nc2ccccc2B(O)O)c2oc(N3CCC(C)(C)CC3)c(C)c(=O)c2c1 ; Cc1cc(C(C)Nc2cccc(F)c2B(O)O)c2oc(N3CCC(C)(C)CC3)c(C)c(=O)c2c1 ; Cc1cc([C@H](C)Nc2ccc(Cl)cc2B(O)O)c2oc(N3CCC(C)(C)CC3)c(C)c(=O)c2c1] 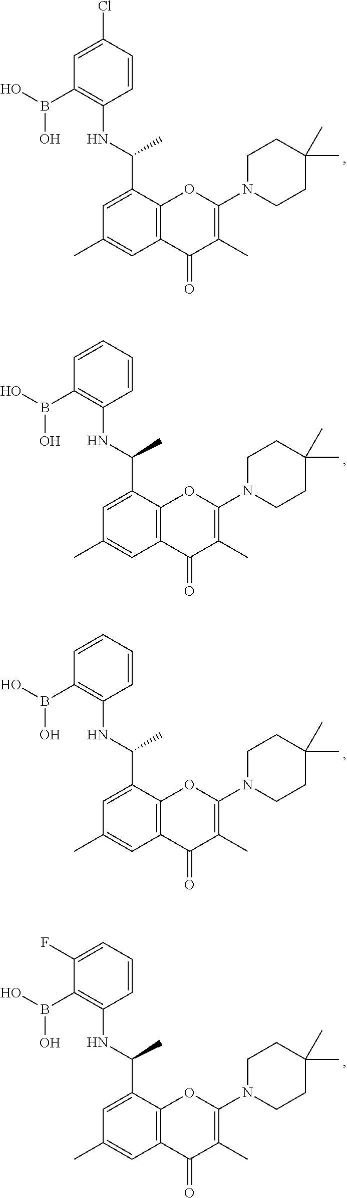 [SMILES: Cc1cc([C@@H](C)Nc2ccc(Cl)cc2B(O)O)c2oc(N3CCC(C)(C)CC3)c(C)c(=O)c2c1 ; Cc1cc([C@H](C)Nc2ccccc2B(O)O)c2oc(N3CCC(C)(C)CC3)c(C)c(=O)c2c1 ; Cc1cc([C@@H](C)Nc2ccccc2B(O)O)c2oc(N3CCC(C)(C)CC3)c(C)c(=O)c2c1 ; Cc1cc([C@H](C)Nc2cccc(F)c2B(O)O)c2oc(N3CCC(C)(C)CC3)c(C)c(=O)c2c1] 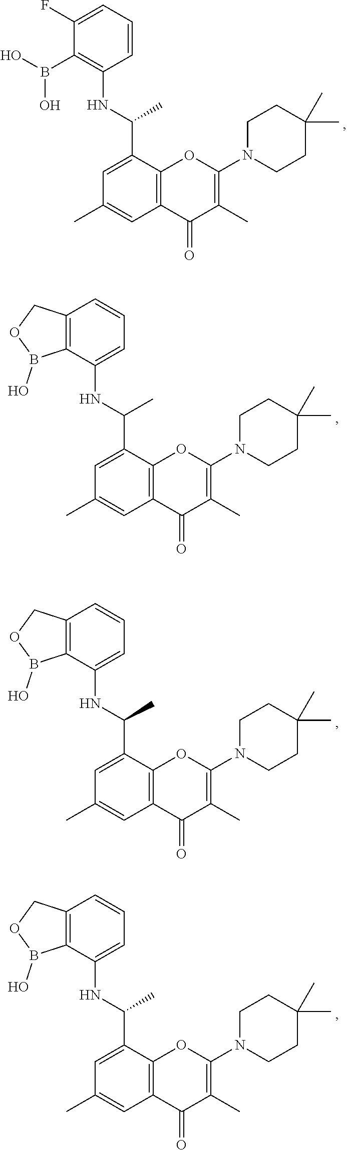 [SMILES: Cc1cc([C@@H](C)Nc2cccc(F)c2B(O)O)c2oc(N3CCC(C)(C)CC3)c(C)c(=O)c2c1 ; Cc1cc(C(C)Nc2cccc3c2B(O)OC3)c2oc(N3CCC(C)(C)CC3)c(C)c(=O)c2c1 ; Cc1cc([C@H](C)Nc2cccc3c2B(O)OC3)c2oc(N3CCC(C)(C)CC3)c(C)c(=O)c2c1 ; Cc1cc([C@@H](C)Nc2cccc3c2B(O)OC3)c2oc(N3CCC(C)(C)CC3)c(C)c(=O)c2c1] 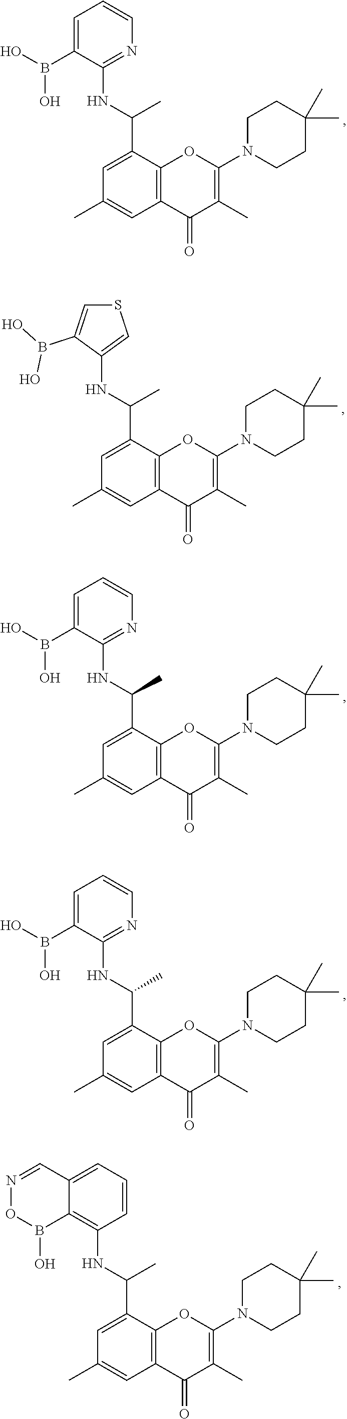 [SMILES: Cc1cc(C(C)Nc2ncccc2B(O)O)c2oc(N3CCC(C)(C)CC3)c(C)c(=O)c2c1 ; Cc1cc(C(C)Nc2cscc2B(O)O)c2oc(N3CCC(C)(C)CC3)c(C)c(=O)c2c1 ; Cc1cc([C@H](C)Nc2ncccc2B(O)O)c2oc(N3CCC(C)(C)CC3)c(C)c(=O)c2c1 ; Cc1cc([C@@H](C)Nc2ncccc2B(O)O)c2oc(N3CCC(C)(C)CC3)c(C)c(=O)c2c1 ; Cc1cc(C(C)Nc2cccc3c2B(O)ON=C3)c2oc(N3CCC(C)(C)CC3)c(C)c(=O)c2c1] 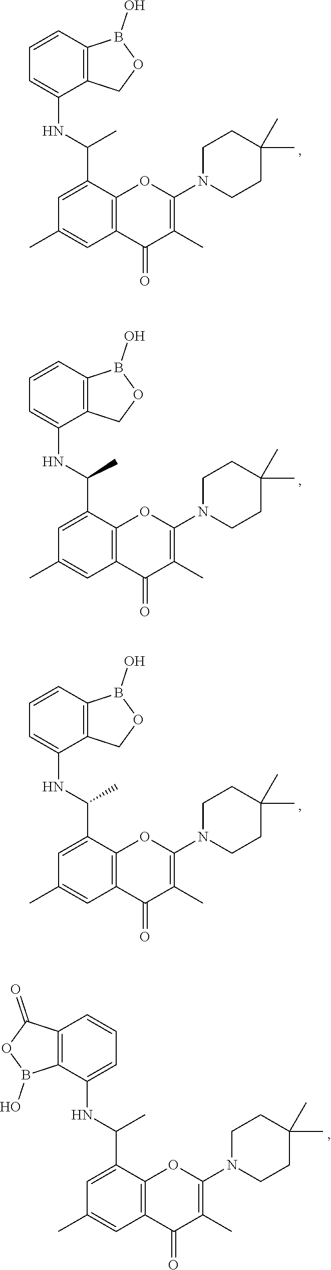 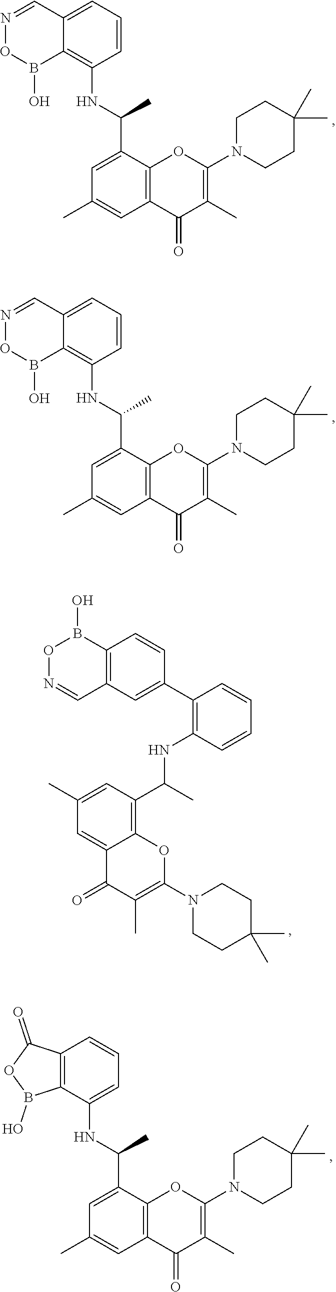 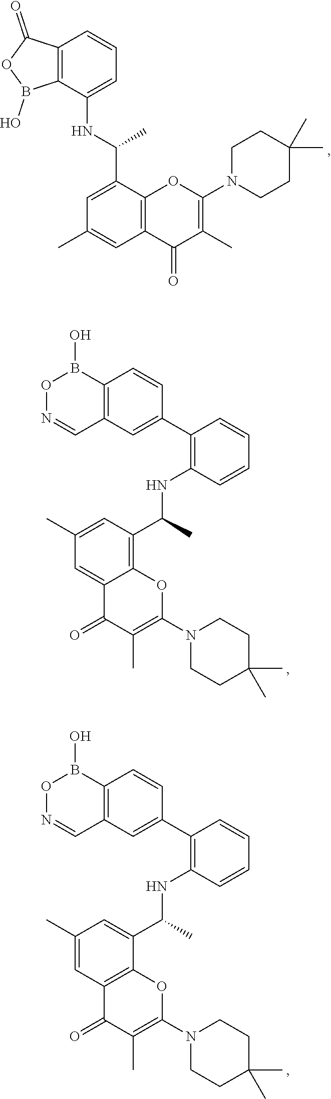 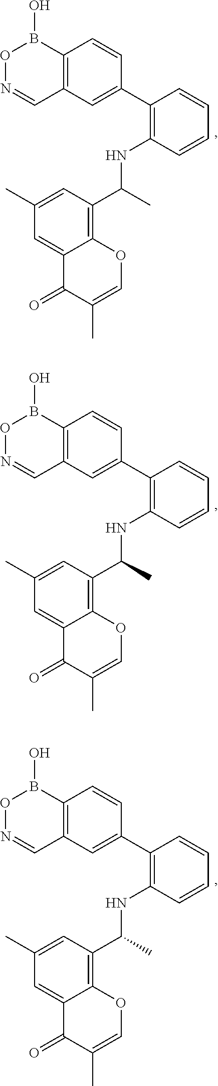 [SMILES: Cc1cc(C(C)Nc2ccccc2-c2ccc3c(c2)C=NOB3O)c2occ(C)c(=O)c2c1 ; Cc1cc([C@H](C)Nc2ccccc2-c2ccc3c(c2)C=NOB3O)c2occ(C)c(=O)c2c1 ; Cc1cc([C@@H](C)Nc2ccccc2-c2ccc3c(c2)C=NOB3O)c2occ(C)c(=O)c2c1] 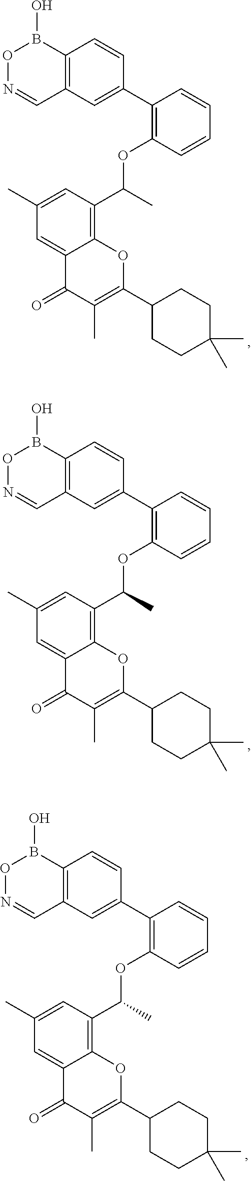 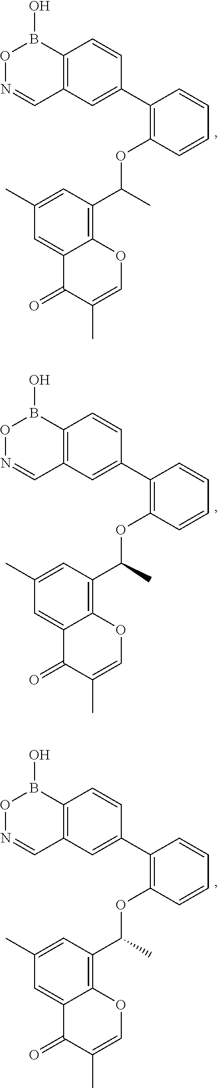 [SMILES: Cc1cc(C(C)Oc2ccccc2-c2ccc3c(c2)C=NOB3O)c2occ(C)c(=O)c2c1 ; Cc1cc([C@H](C)Oc2ccccc2-c2ccc3c(c2)C=NOB3O)c2occ(C)c(=O)c2c1 ; Cc1cc([C@@H](C)Oc2ccccc2-c2ccc3c(c2)C=NOB3O)c2occ(C)c(=O)c2c1] 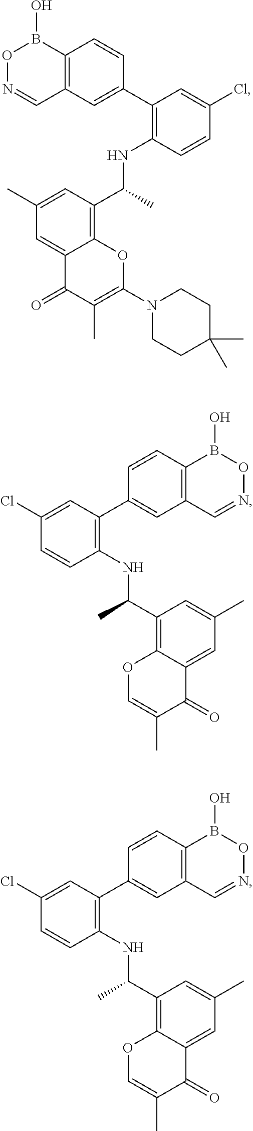 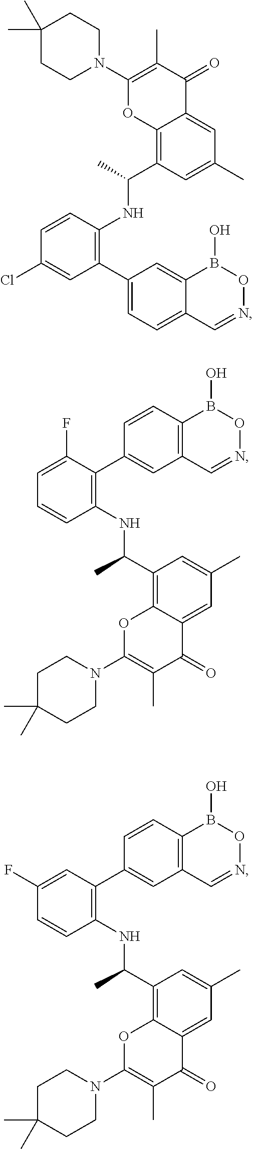 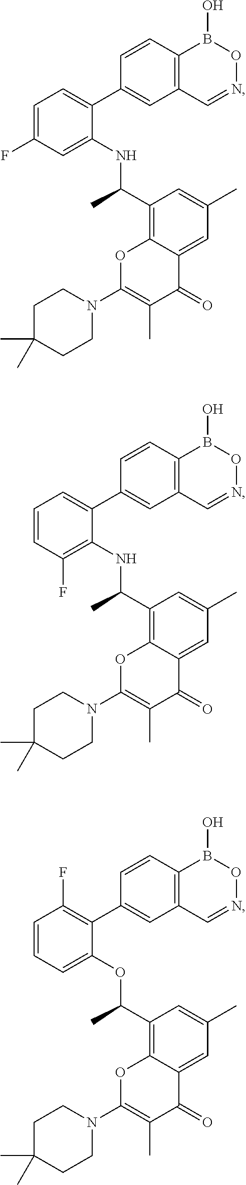 [SMILES: Cc1cc([C@@H](C)Nc2cc(F)ccc2-c2ccc3c(c2)C=NOB3O)c2oc(N3CCC(C)(C)CC3)c(C)c(=O)c2c1 ; Cc1cc([C@@H](C)Nc2c(F)cccc2-c2ccc3c(c2)C=NOB3O)c2oc(N3CCC(C)(C)CC3)c(C)c(=O)c2c1 ; Cc1cc([C@@H](C)Oc2cccc(F)c2-c2ccc3c(c2)C=NOB3O)c2oc(N3CCC(C)(C)CC3)c(C)c(=O)c2c1] 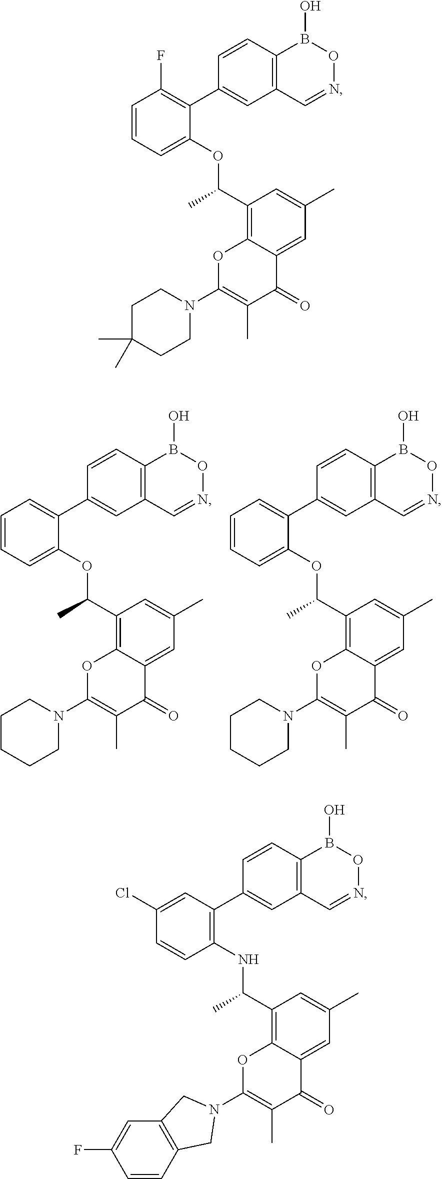 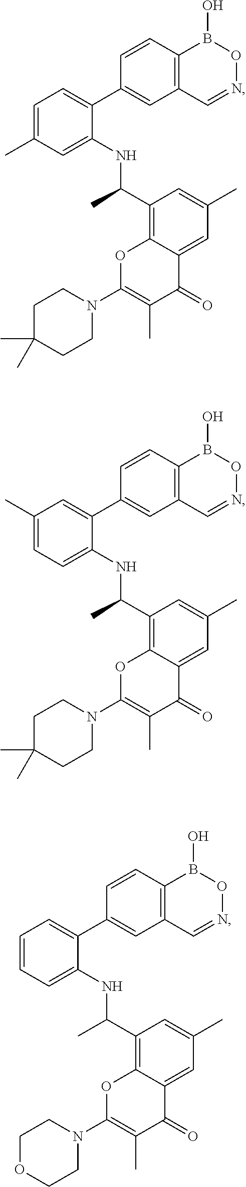 [SMILES: Cc1ccc(-c2ccc3c(c2)C=NOB3O)c(N[C@H](C)c2cc(C)cc3c(=O)c(C)c(N4CCC(C)(C)CC4)oc23)c1 ; Cc1ccc(N[C@H](C)c2cc(C)cc3c(=O)c(C)c(N4CCC(C)(C)CC4)oc23)c(-c2ccc3c(c2)C=NOB3O)c1 ; Cc1cc(C(C)Nc2ccccc2-c2ccc3c(c2)C=NOB3O)c2oc(N3CCOCC3)c(C)c(=O)c2c1] 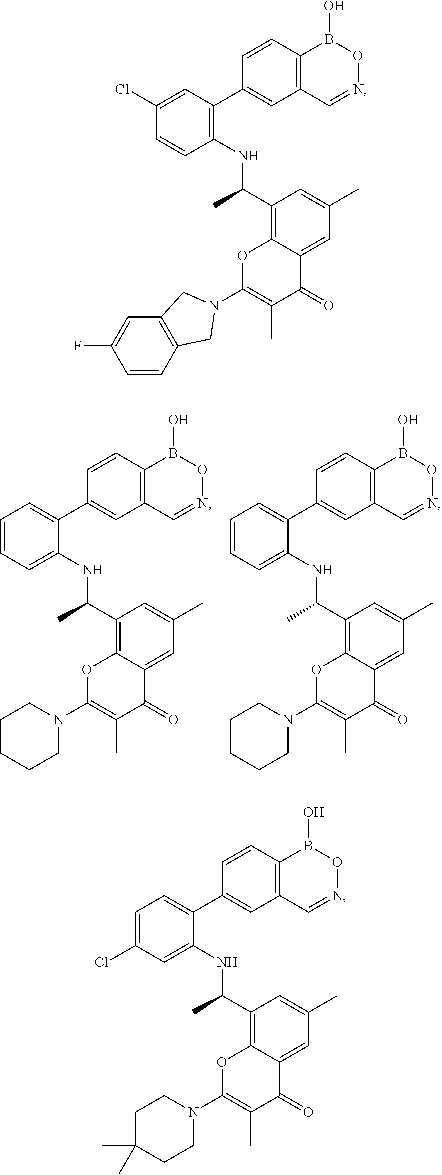 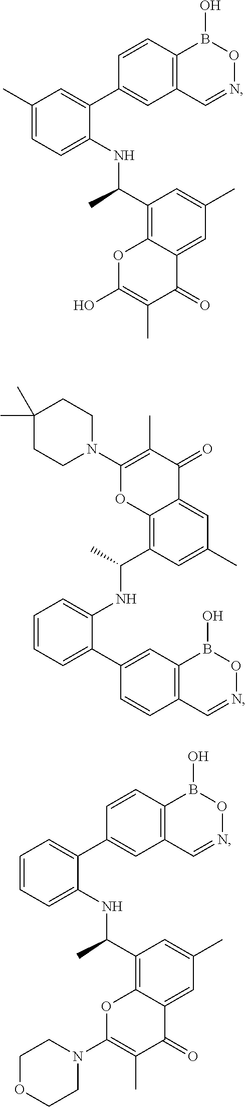 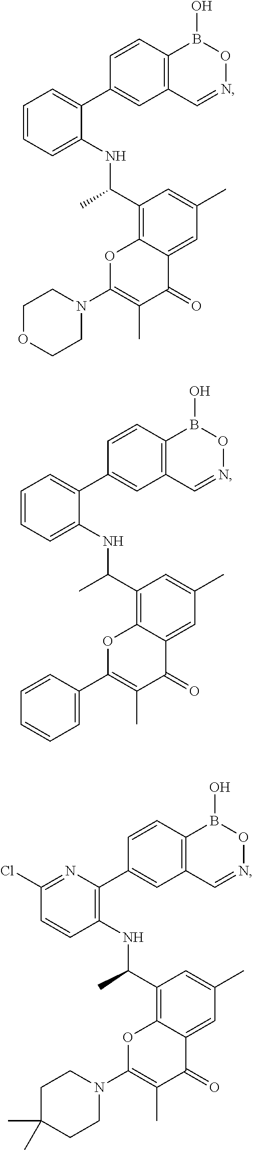 [SMILES: Cc1cc([C@H](C)Nc2ccccc2-c2ccc3c(c2)C=NOB3O)c2oc(N3CCOCC3)c(C)c(=O)c2c1 ; Cc1cc(C(C)Nc2ccccc2-c2ccc3c(c2)C=NOB3O)c2oc(-c3ccccc3)c(C)c(=O)c2c1 ; Cc1cc([C@@H](C)Nc2ccc(Cl)nc2-c2ccc3c(c2)C=NOB3O)c2oc(N3CCC(C)(C)CC3)c(C)c(=O)c2c1] 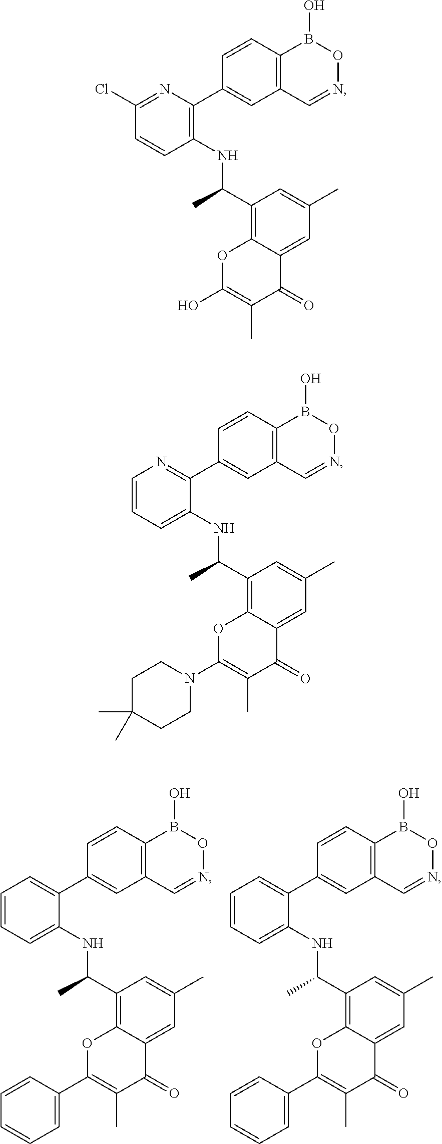 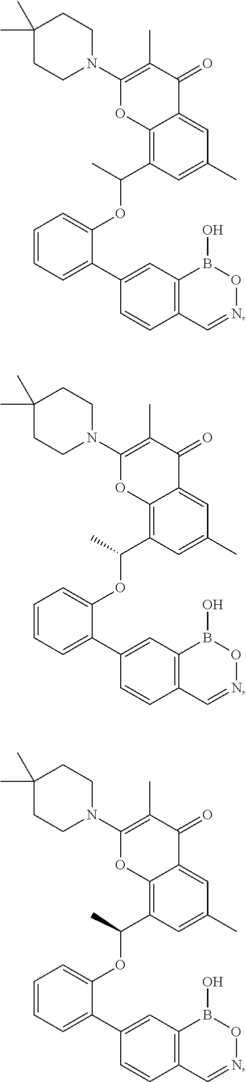 [SMILES: Cc1cc(C(C)Oc2ccccc2-c2ccc3c(c2)B(O)ON=C3)c2oc(N3CCC(C)(C)CC3)c(C)c(=O)c2c1 ; Cc1cc([C@@H](C)Oc2ccccc2-c2ccc3c(c2)B(O)ON=C3)c2oc(N3CCC(C)(C)CC3)c(C)c(=O)c2c1 ; Cc1cc([C@H](C)Oc2ccccc2-c2ccc3c(c2)B(O)ON=C3)c2oc(N3CCC(C)(C)CC3)c(C)c(=O)c2c1] 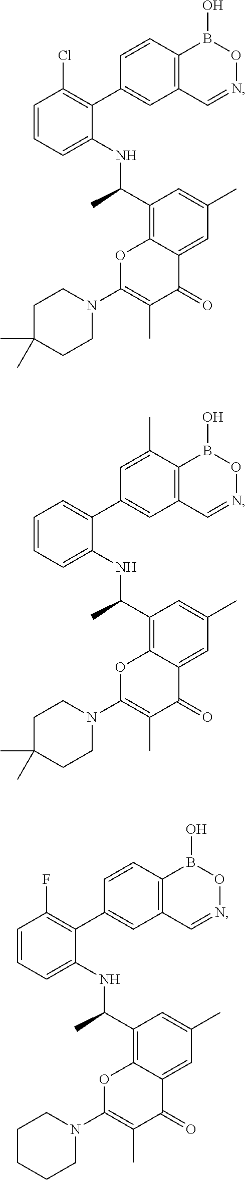 [SMILES: Cc1cc([C@@H](C)Nc2cccc(Cl)c2-c2ccc3c(c2)C=NOB3O)c2oc(N3CCC(C)(C)CC3)c(C)c(=O)c2c1 ; Cc1cc([C@@H](C)Nc2ccccc2-c2cc(C)c3c(c2)C=NOB3O)c2oc(N3CCC(C)(C)CC3)c(C)c(=O)c2c1 ; Cc1cc([C@@H](C)Nc2cccc(F)c2-c2ccc3c(c2)C=NOB3O)c2oc(N3CCCCC3)c(C)c(=O)c2c1] 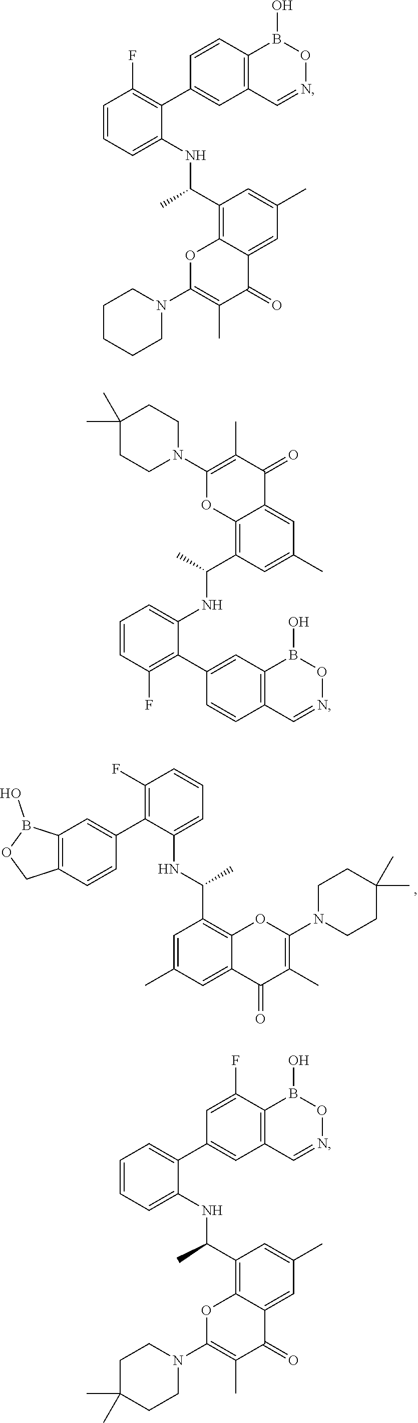 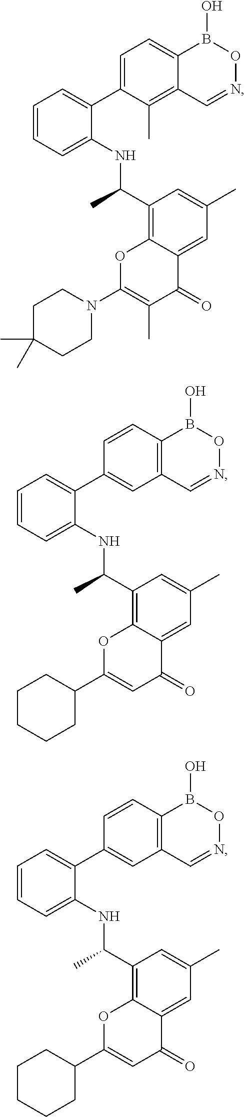 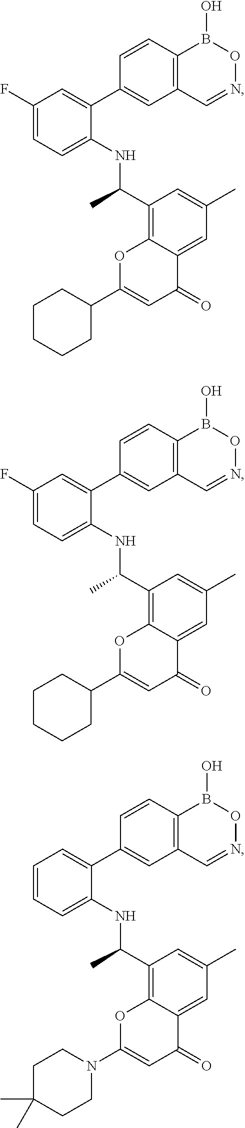 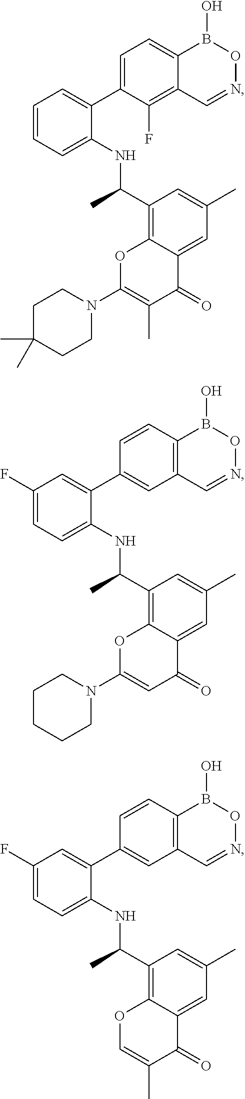 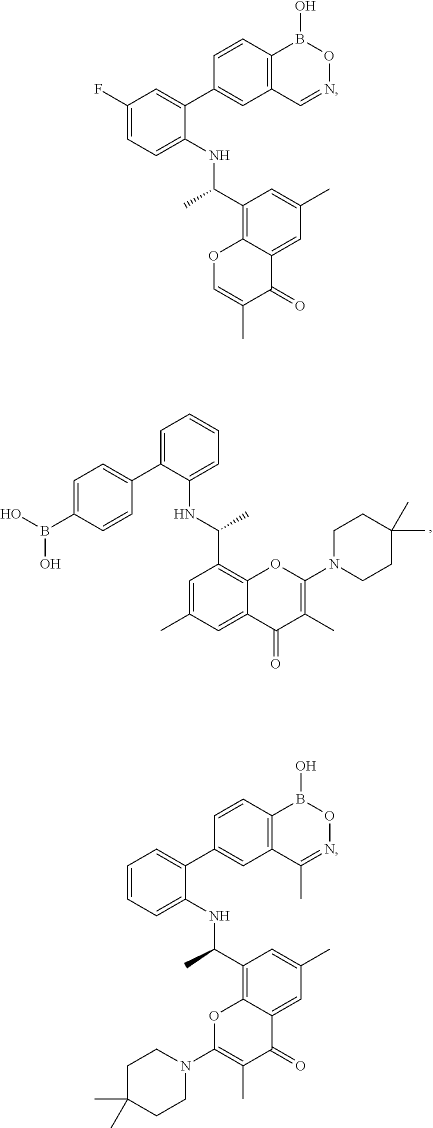 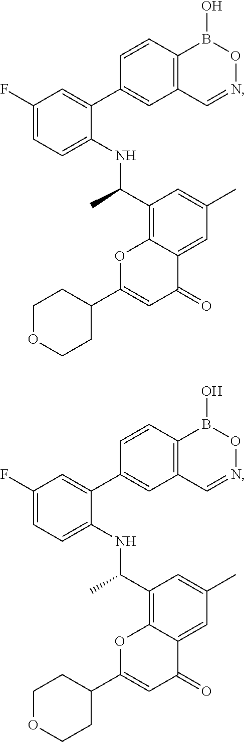 [SMILES: Cc1cc([C@@H](C)Nc2ccc(F)cc2-c2ccc3c(c2)C=NOB3O)c2oc(C3CCOCC3)cc(=O)c2c1 ; Cc1cc([C@H](C)Nc2ccc(F)cc2-c2ccc3c(c2)C=NOB3O)c2oc(C3CCOCC3)cc(=O)c2c1] 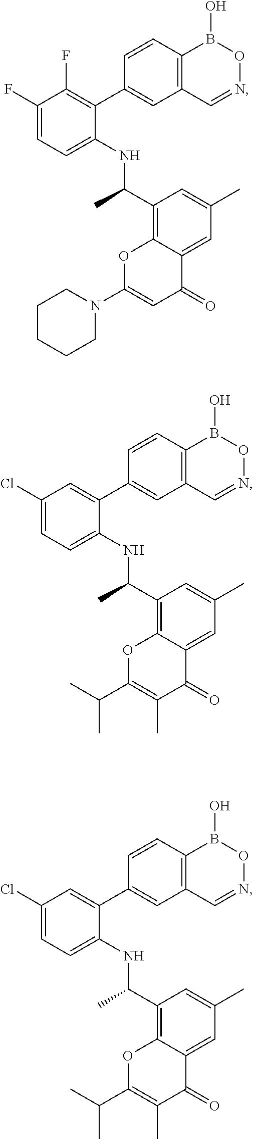 [SMILES: Cc1cc([C@@H](C)Nc2ccc(F)c(F)c2-c2ccc3c(c2)C=NOB3O)c2oc(N3CCCCC3)cc(=O)c2c1 ; Cc1cc([C@@H](C)Nc2ccc(Cl)cc2-c2ccc3c(c2)C=NOB3O)c2oc(C(C)C)c(C)c(=O)c2c1 ; Cc1cc([C@H](C)Nc2ccc(Cl)cc2-c2ccc3c(c2)C=NOB3O)c2oc(C(C)C)c(C)c(=O)c2c1] 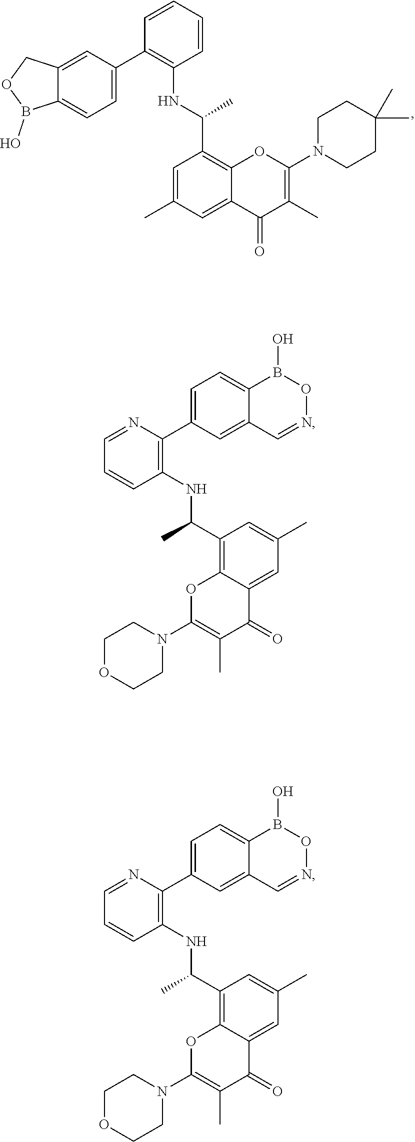 [SMILES: Cc1cc([C@@H](C)Nc2ccccc2-c2ccc3c(c2)COB3O)c2oc(N3CCC(C)(C)CC3)c(C)c(=O)c2c1 ; Cc1cc([C@@H](C)Nc2cccnc2-c2ccc3c(c2)C=NOB3O)c2oc(N3CCOCC3)c(C)c(=O)c2c1 ; Cc1cc([C@H](C)Nc2cccnc2-c2ccc3c(c2)C=NOB3O)c2oc(N3CCOCC3)c(C)c(=O)c2c1] 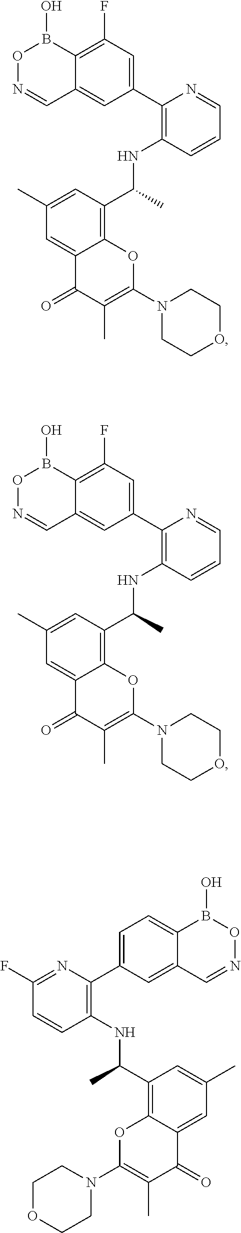 [SMILES: Cc1cc([C@@H](C)Nc2cccnc2-c2cc(F)c3c(c2)C=NOB3O)c2oc(N3CCOCC3)c(C)c(=O)c2c1 ; Cc1cc([C@H](C)Nc2cccnc2-c2cc(F)c3c(c2)C=NOB3O)c2oc(N3CCOCC3)c(C)c(=O)c2c1 ; Cc1cc([C@@H](C)Nc2ccc(F)nc2-c2ccc3c(c2)C=NOB3O)c2oc(N3CCOCC3)c(C)c(=O)c2c1] 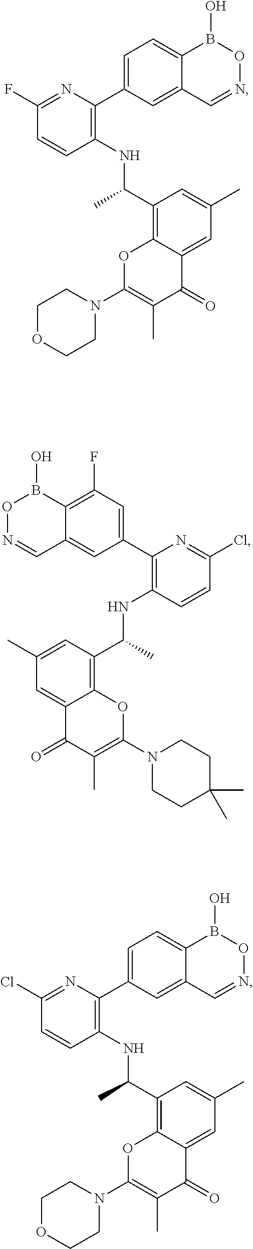 [SMILES: Cc1cc([C@H](C)Nc2ccc(F)nc2-c2ccc3c(c2)C=NOB3O)c2oc(N3CCOCC3)c(C)c(=O)c2c1 ; Cc1cc([C@@H](C)Nc2ccc(Cl)nc2-c2cc(F)c3c(c2)C=NOB3O)c2oc(N3CCC(C)(C)CC3)c(C)c(=O)c2c1 ; Cc1cc([C@@H](C)Nc2ccc(Cl)nc2-c2ccc3c(c2)C=NOB3O)c2oc(N3CCOCC3)c(C)c(=O)c2c1] 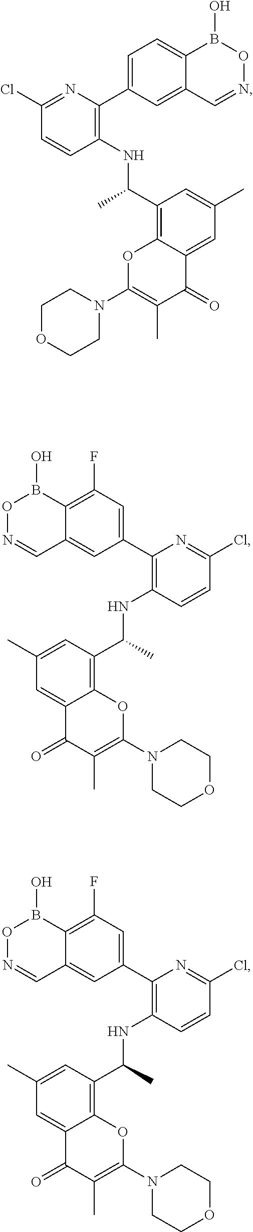 [SMILES: Cc1cc([C@H](C)Nc2ccc(Cl)nc2-c2ccc3c(c2)C=NOB3O)c2oc(N3CCOCC3)c(C)c(=O)c2c1 ; Cc1cc([C@@H](C)Nc2ccc(Cl)nc2-c2cc(F)c3c(c2)C=NOB3O)c2oc(N3CCOCC3)c(C)c(=O)c2c1 ; Cc1cc([C@H](C)Nc2ccc(Cl)nc2-c2cc(F)c3c(c2)C=NOB3O)c2oc(N3CCOCC3)c(C)c(=O)c2c1] 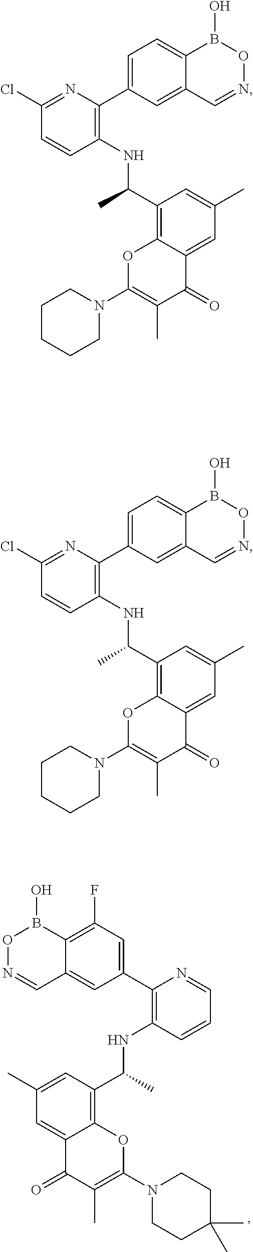 [SMILES: Cc1cc([C@@H](C)Nc2ccc(Cl)nc2-c2ccc3c(c2)C=NOB3O)c2oc(N3CCCCC3)c(C)c(=O)c2c1 ; Cc1cc([C@H](C)Nc2ccc(Cl)nc2-c2ccc3c(c2)C=NOB3O)c2oc(N3CCCCC3)c(C)c(=O)c2c1 ; Cc1cc([C@@H](C)Nc2cccnc2-c2cc(F)c3c(c2)C=NOB3O)c2oc(N3CCC(C)(C)CC3)c(C)c(=O)c2c1] 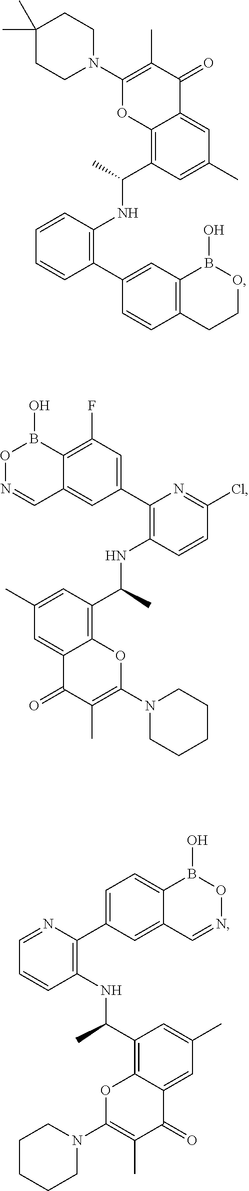 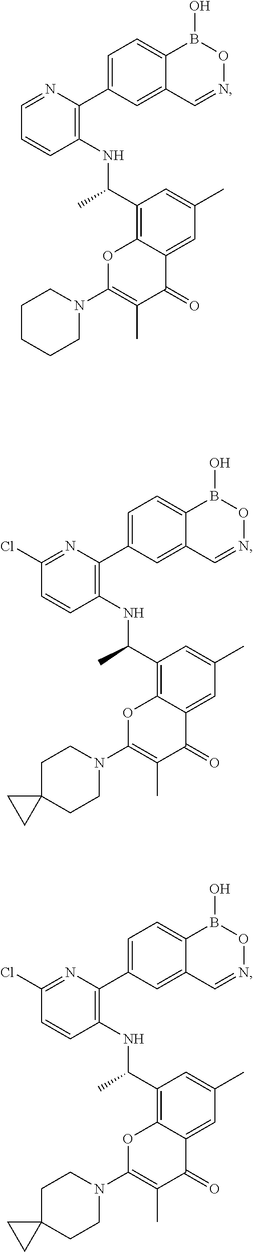 [SMILES: Cc1cc([C@H](C)Nc2cccnc2-c2ccc3c(c2)C=NOB3O)c2oc(N3CCCCC3)c(C)c(=O)c2c1 ; Cc1cc([C@@H](C)Nc2ccc(Cl)nc2-c2ccc3c(c2)C=NOB3O)c2oc(N3CCC4(CC3)CC4)c(C)c(=O)c2c1 ; Cc1cc([C@H](C)Nc2ccc(Cl)nc2-c2ccc3c(c2)C=NOB3O)c2oc(N3CCC4(CC3)CC4)c(C)c(=O)c2c1] 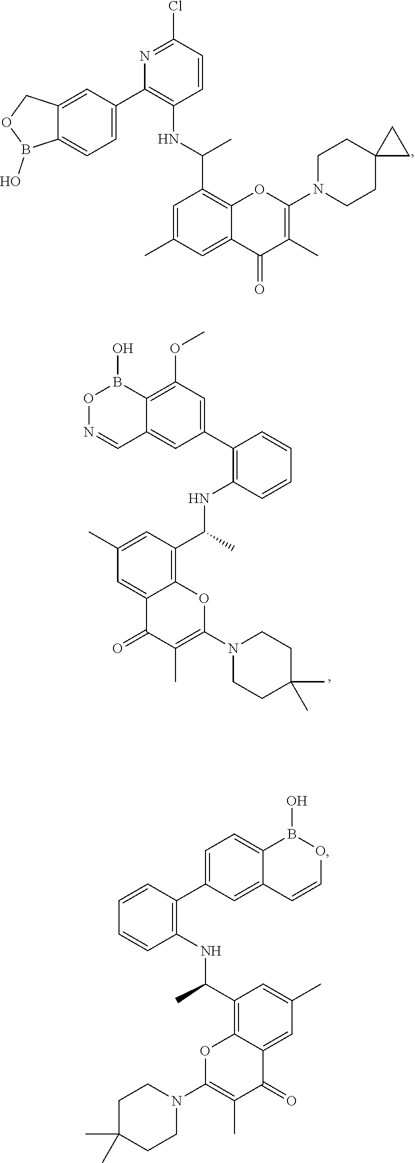 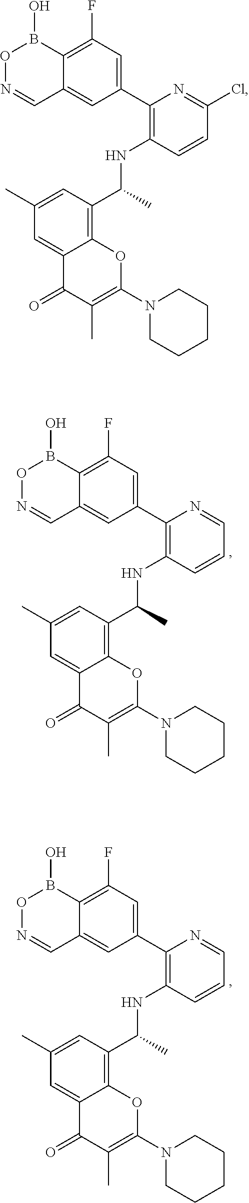 [SMILES: Cc1cc([C@@H](C)Nc2ccc(Cl)nc2-c2cc(F)c3c(c2)C=NOB3O)c2oc(N3CCCCC3)c(C)c(=O)c2c1 ; Cc1cc([C@H](C)Nc2cccnc2-c2cc(F)c3c(c2)C=NOB3O)c2oc(N3CCCCC3)c(C)c(=O)c2c1 ; Cc1cc([C@@H](C)Nc2cccnc2-c2cc(F)c3c(c2)C=NOB3O)c2oc(N3CCCCC3)c(C)c(=O)c2c1] 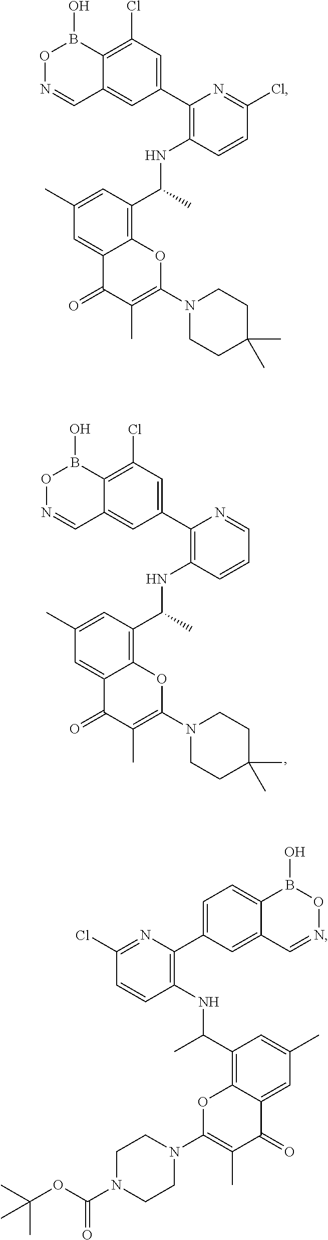 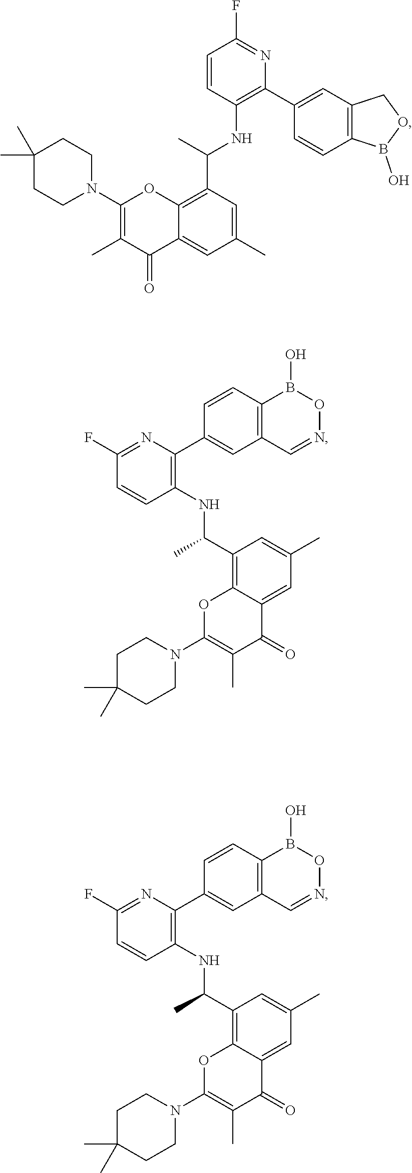 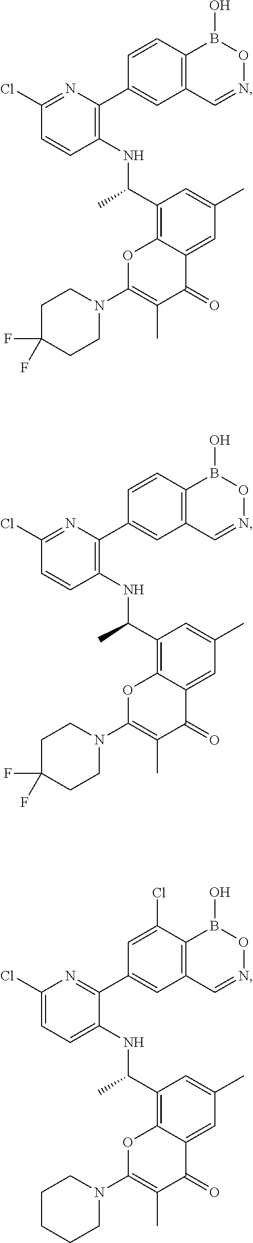 [SMILES: Cc1cc([C@H](C)Nc2ccc(Cl)nc2-c2ccc3c(c2)C=NOB3O)c2oc(N3CCC(F)(F)CC3)c(C)c(=O)c2c1 ; Cc1cc([C@@H](C)Nc2ccc(Cl)nc2-c2ccc3c(c2)C=NOB3O)c2oc(N3CCC(F)(F)CC3)c(C)c(=O)c2c1 ; Cc1cc([C@H](C)Nc2ccc(Cl)nc2-c2cc(Cl)c3c(c2)C=NOB3O)c2oc(N3CCCCC3)c(C)c(=O)c2c1] 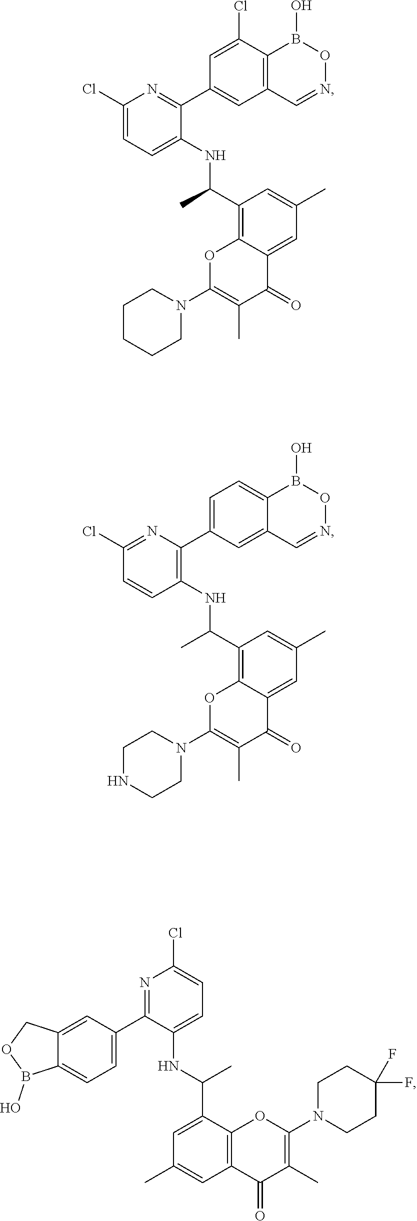 [SMILES: Cc1cc([C@@H](C)Nc2ccc(Cl)nc2-c2cc(Cl)c3c(c2)C=NOB3O)c2oc(N3CCCCC3)c(C)c(=O)c2c1 ; Cc1cc(C(C)Nc2ccc(Cl)nc2-c2ccc3c(c2)C=NOB3O)c2oc(N3CCNCC3)c(C)c(=O)c2c1 ; Cc1cc(C(C)Nc2ccc(Cl)nc2-c2ccc3c(c2)COB3O)c2oc(N3CCC(F)(F)CC3)c(C)c(=O)c2c1] 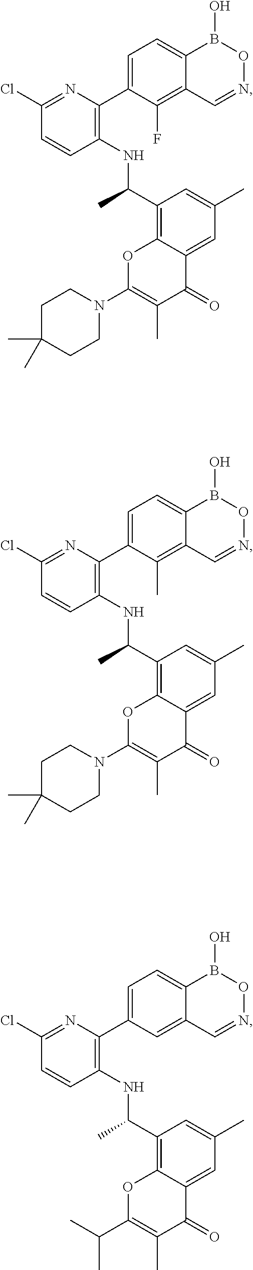 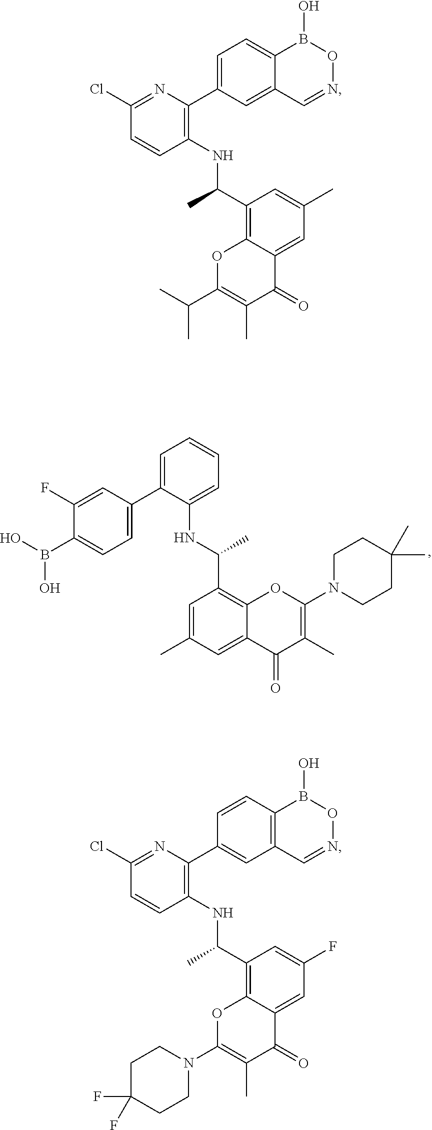 [SMILES: Cc1cc([C@@H](C)Nc2ccc(Cl)nc2-c2ccc3c(c2)C=NOB3O)c2oc(C(C)C)c(C)c(=O)c2c1 ; Cc1cc([C@@H](C)Nc2ccccc2-c2ccc(B(O)O)c(F)c2)c2oc(N3CCC(C)(C)CC3)c(C)c(=O)c2c1 ; Cc1c(N2CCC(F)(F)CC2)oc2c([C@H](C)Nc3ccc(Cl)nc3-c3ccc4c(c3)C=NOB4O)cc(F)cc2c1=O] 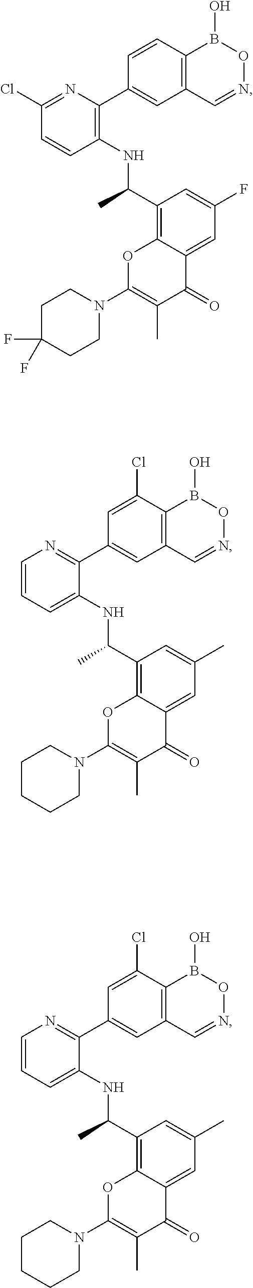 [SMILES: Cc1c(N2CCC(F)(F)CC2)oc2c([C@@H](C)Nc3ccc(Cl)nc3-c3ccc4c(c3)C=NOB4O)cc(F)cc2c1=O ; Cc1cc([C@H](C)Nc2cccnc2-c2cc(Cl)c3c(c2)C=NOB3O)c2oc(N3CCCCC3)c(C)c(=O)c2c1 ; Cc1cc([C@@H](C)Nc2cccnc2-c2cc(Cl)c3c(c2)C=NOB3O)c2oc(N3CCCCC3)c(C)c(=O)c2c1] 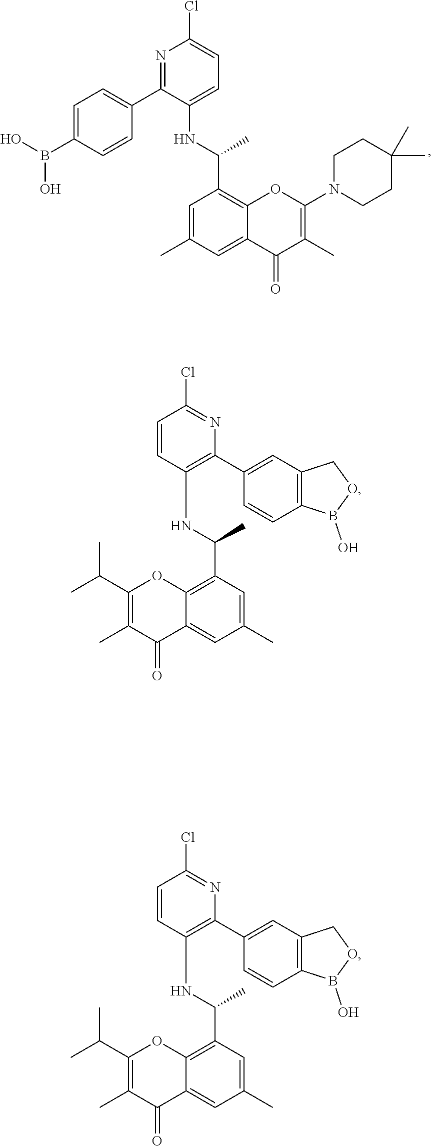 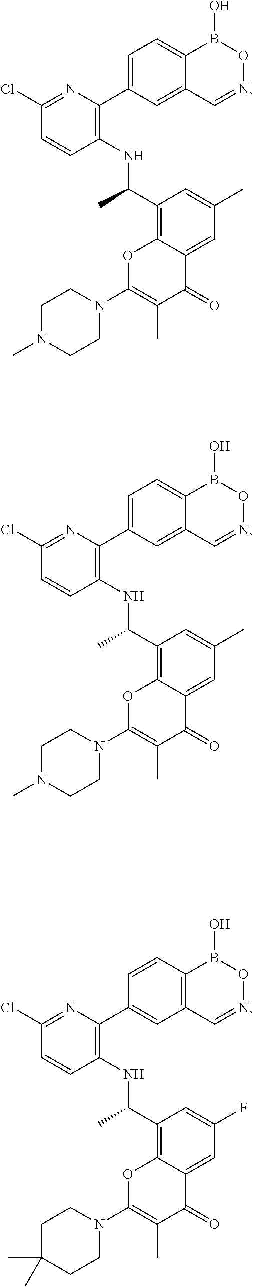 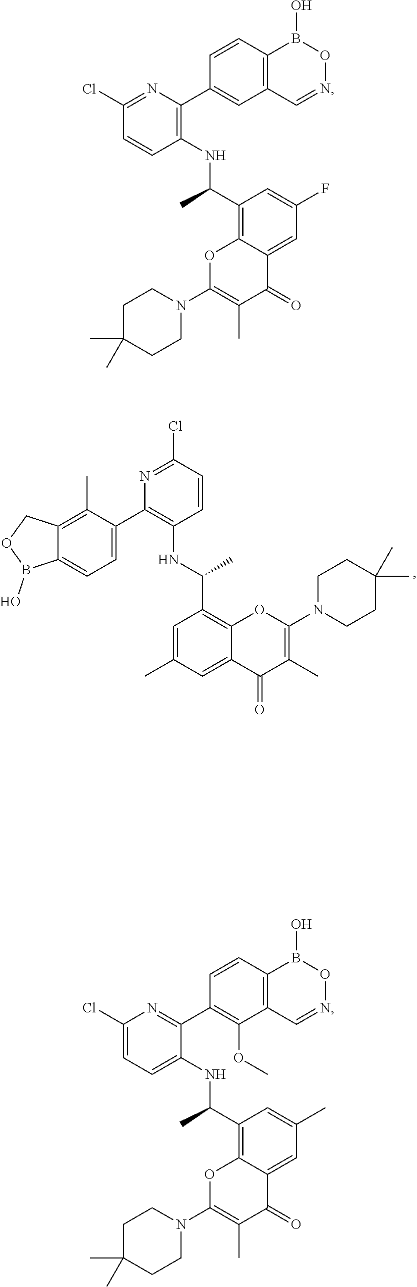 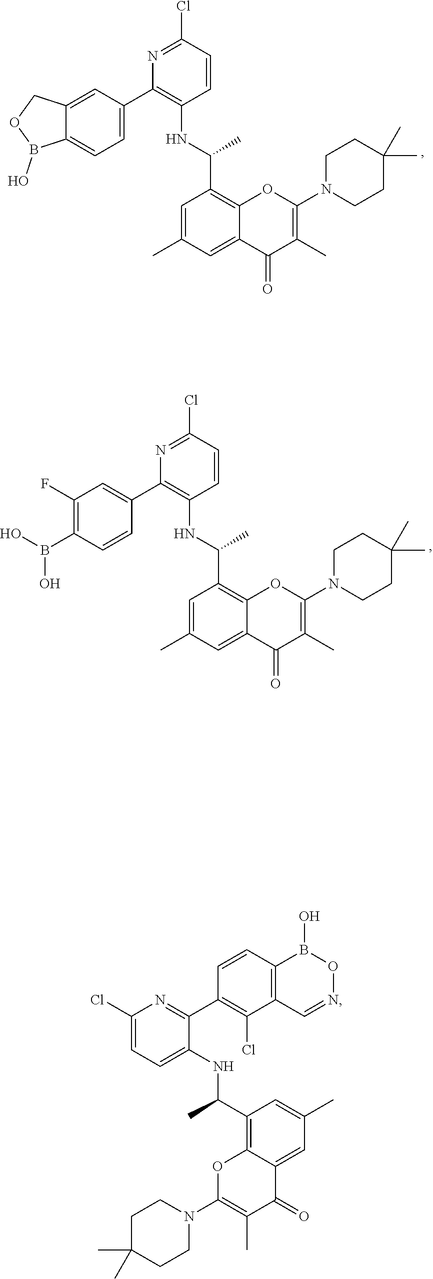 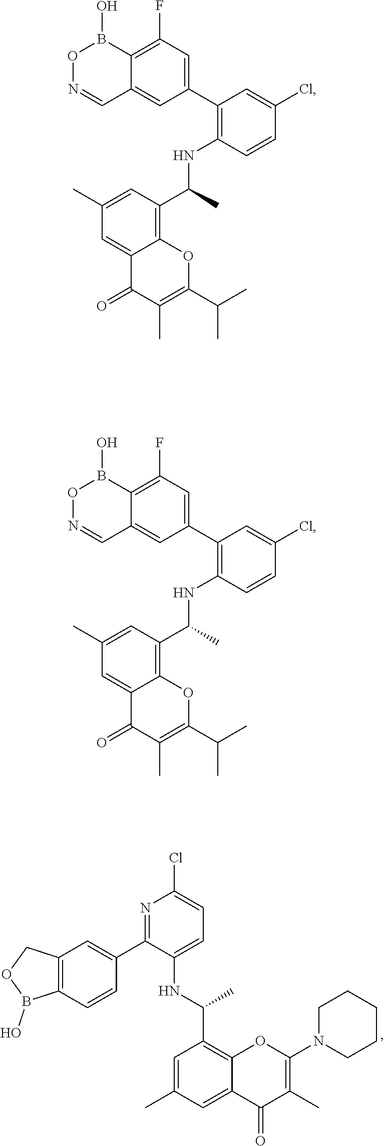 [SMILES: Cc1cc([C@H](C)Nc2ccc(Cl)cc2-c2cc(F)c3c(c2)C=NOB3O)c2oc(C(C)C)c(C)c(=O)c2c1 ; Cc1cc([C@@H](C)Nc2ccc(Cl)cc2-c2cc(F)c3c(c2)C=NOB3O)c2oc(C(C)C)c(C)c(=O)c2c1 ; Cc1cc([C@@H](C)Nc2ccc(Cl)nc2-c2ccc3c(c2)COB3O)c2oc(N3CCCCC3)c(C)c(=O)c2c1] 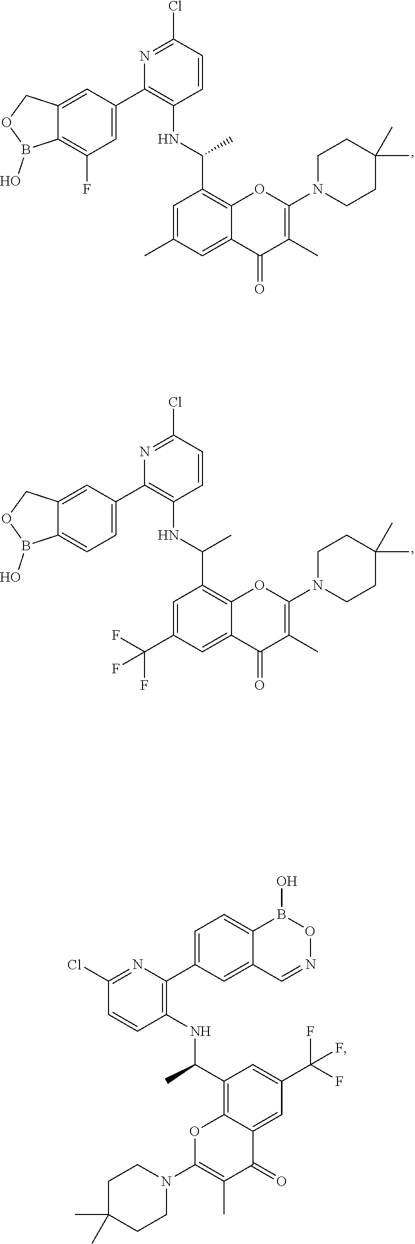 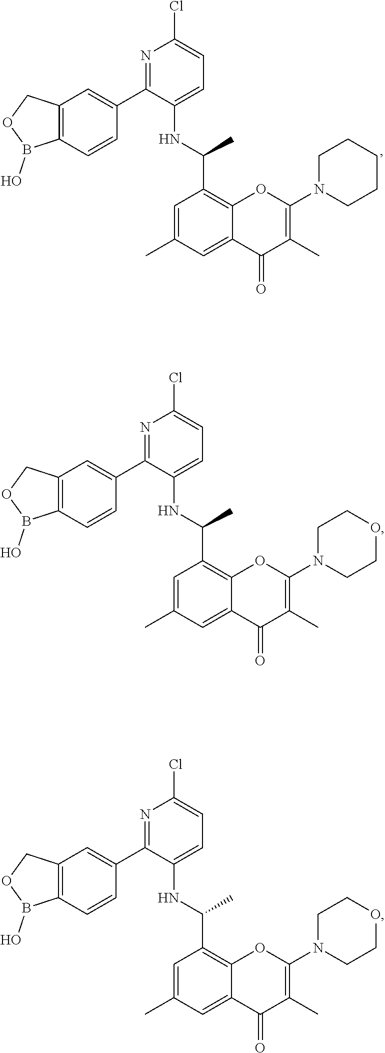 [SMILES: Cc1cc([C@H](C)Nc2ccc(Cl)nc2-c2ccc3c(c2)COB3O)c2oc(N3CCCCC3)c(C)c(=O)c2c1 ; Cc1cc([C@H](C)Nc2ccc(Cl)nc2-c2ccc3c(c2)COB3O)c2oc(N3CCOCC3)c(C)c(=O)c2c1 ; Cc1cc([C@@H](C)Nc2ccc(Cl)nc2-c2ccc3c(c2)COB3O)c2oc(N3CCOCC3)c(C)c(=O)c2c1] 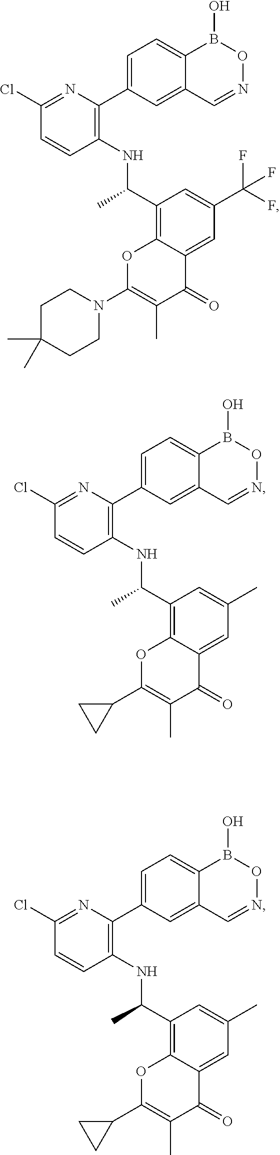 [SMILES: Cc1c(N2CCC(C)(C)CC2)oc2c([C@H](C)Nc3ccc(Cl)nc3-c3ccc4c(c3)C=NOB4O)cc(C(F)(F)F)cc2c1=O ; Cc1cc([C@H](C)Nc2ccc(Cl)nc2-c2ccc3c(c2)C=NOB3O)c2oc(C3CC3)c(C)c(=O)c2c1 ; Cc1cc([C@@H](C)Nc2ccc(Cl)nc2-c2ccc3c(c2)C=NOB3O)c2oc(C3CC3)c(C)c(=O)c2c1] 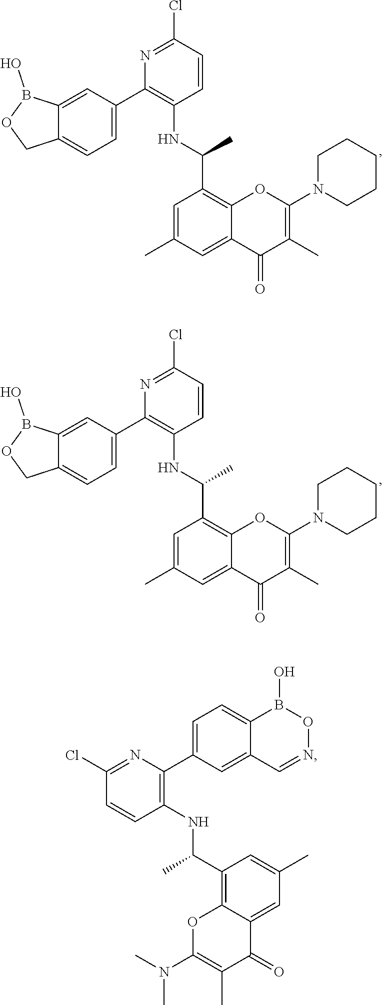 [SMILES: Cc1cc([C@H](C)Nc2ccc(Cl)nc2-c2ccc3c(c2)B(O)OC3)c2oc(N3CCCCC3)c(C)c(=O)c2c1 ; Cc1cc([C@@H](C)Nc2ccc(Cl)nc2-c2ccc3c(c2)B(O)OC3)c2oc(N3CCCCC3)c(C)c(=O)c2c1 ; Cc1cc([C@H](C)Nc2ccc(Cl)nc2-c2ccc3c(c2)C=NOB3O)c2oc(N(C)C)c(C)c(=O)c2c1] 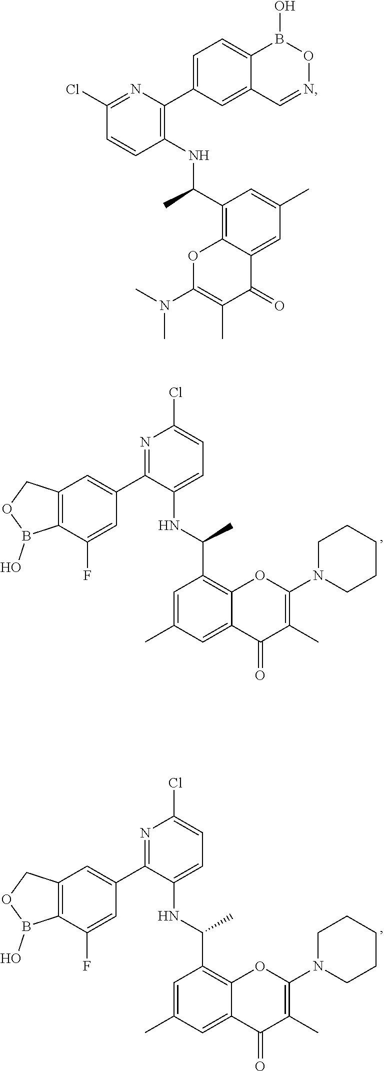 [SMILES: Cc1cc([C@@H](C)Nc2ccc(Cl)nc2-c2ccc3c(c2)C=NOB3O)c2oc(N(C)C)c(C)c(=O)c2c1 ; Cc1cc([C@H](C)Nc2ccc(Cl)nc2-c2cc(F)c3c(c2)COB3O)c2oc(N3CCCCC3)c(C)c(=O)c2c1 ; Cc1cc([C@@H](C)Nc2ccc(Cl)nc2-c2cc(F)c3c(c2)COB3O)c2oc(N3CCCCC3)c(C)c(=O)c2c1] 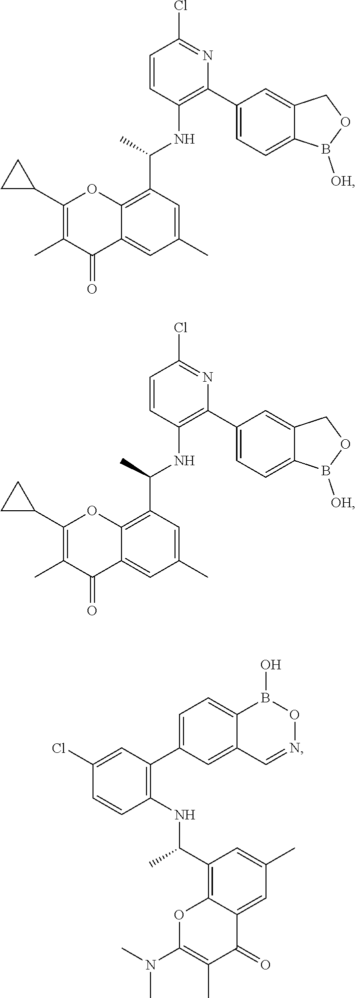 [SMILES: Cc1cc([C@H](C)Nc2ccc(Cl)nc2-c2ccc3c(c2)COB3O)c2oc(C3CC3)c(C)c(=O)c2c1 ; Cc1cc([C@@H](C)Nc2ccc(Cl)nc2-c2ccc3c(c2)COB3O)c2oc(C3CC3)c(C)c(=O)c2c1 ; Cc1cc([C@H](C)Nc2ccc(Cl)cc2-c2ccc3c(c2)C=NOB3O)c2oc(N(C)C)c(C)c(=O)c2c1] 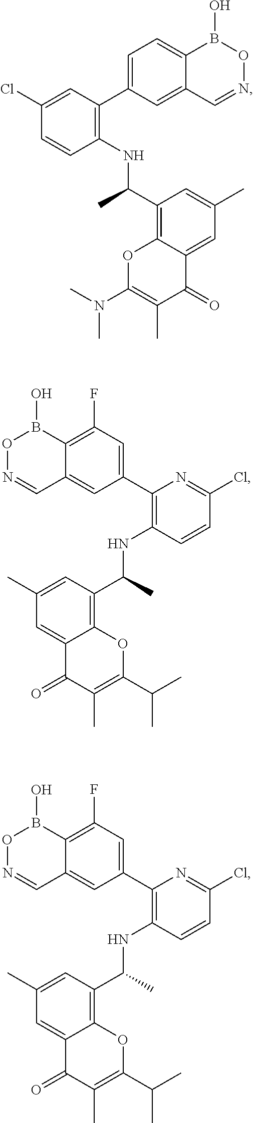 [SMILES: Cc1cc([C@@H](C)Nc2ccc(Cl)cc2-c2ccc3c(c2)C=NOB3O)c2oc(N(C)C)c(C)c(=O)c2c1 ; Cc1cc([C@H](C)Nc2ccc(Cl)nc2-c2cc(F)c3c(c2)C=NOB3O)c2oc(C(C)C)c(C)c(=O)c2c1 ; Cc1cc([C@@H](C)Nc2ccc(Cl)nc2-c2cc(F)c3c(c2)C=NOB3O)c2oc(C(C)C)c(C)c(=O)c2c1] 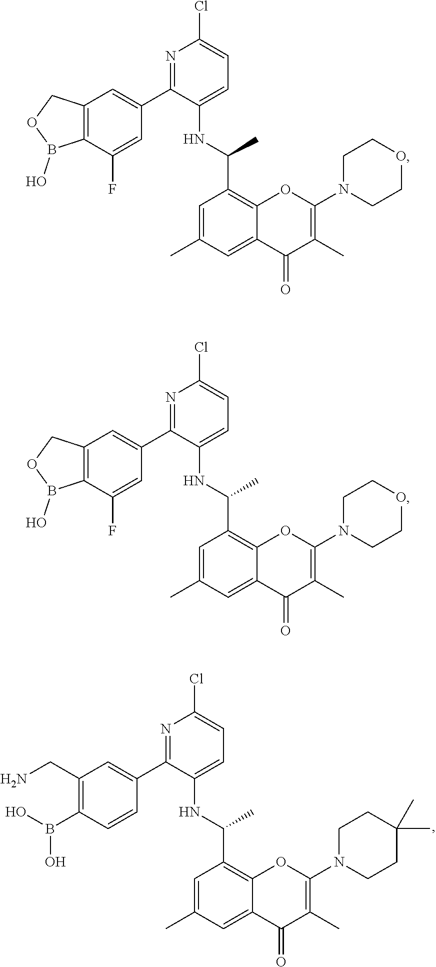 [SMILES: Cc1cc([C@H](C)Nc2ccc(Cl)nc2-c2cc(F)c3c(c2)COB3O)c2oc(N3CCOCC3)c(C)c(=O)c2c1 ; Cc1cc([C@@H](C)Nc2ccc(Cl)nc2-c2cc(F)c3c(c2)COB3O)c2oc(N3CCOCC3)c(C)c(=O)c2c1 ; Cc1cc([C@@H](C)Nc2ccc(Cl)nc2-c2ccc(B(O)O)c(CN)c2)c2oc(N3CCC(C)(C)CC3)c(C)c(=O)c2c1] 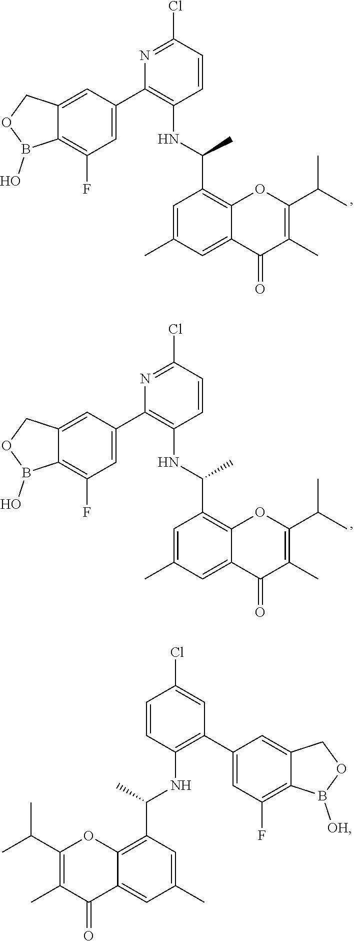 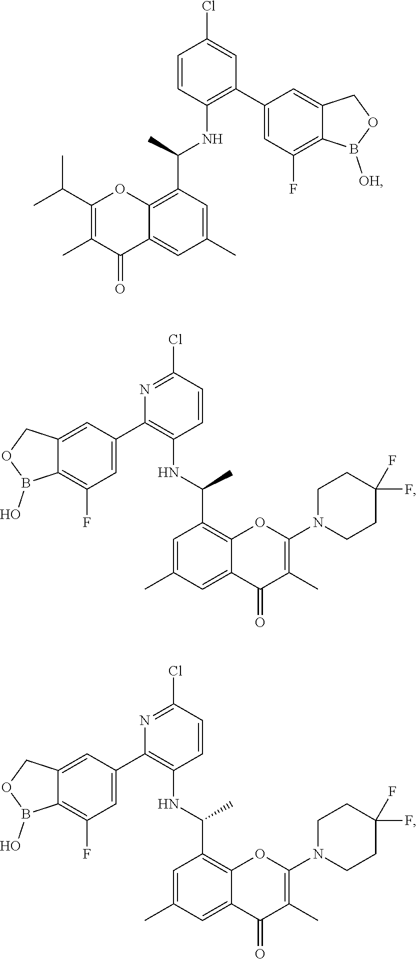 [SMILES: Cc1cc([C@@H](C)Nc2ccc(Cl)cc2-c2cc(F)c3c(c2)COB3O)c2oc(C(C)C)c(C)c(=O)c2c1 ; Cc1cc([C@H](C)Nc2ccc(Cl)nc2-c2cc(F)c3c(c2)COB3O)c2oc(N3CCC(F)(F)CC3)c(C)c(=O)c2c1 ; Cc1cc([C@@H](C)Nc2ccc(Cl)nc2-c2cc(F)c3c(c2)COB3O)c2oc(N3CCC(F)(F)CC3)c(C)c(=O)c2c1] 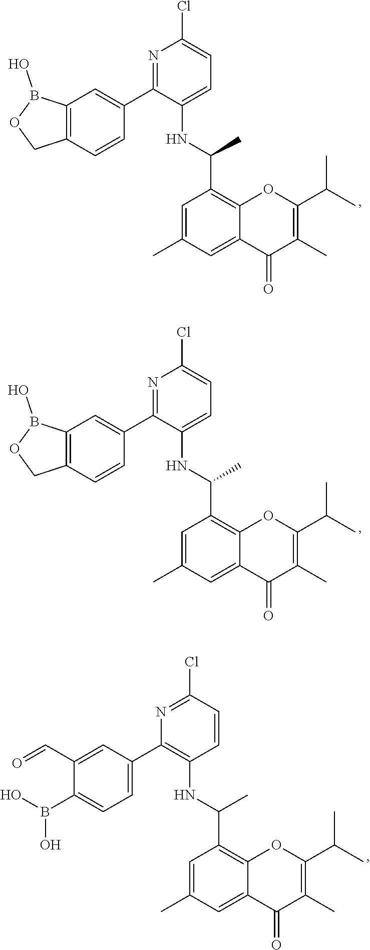 [SMILES: Cc1cc([C@H](C)Nc2ccc(Cl)nc2-c2ccc3c(c2)B(O)OC3)c2oc(C(C)C)c(C)c(=O)c2c1 ; Cc1cc([C@@H](C)Nc2ccc(Cl)nc2-c2ccc3c(c2)B(O)OC3)c2oc(C(C)C)c(C)c(=O)c2c1 ; Cc1cc(C(C)Nc2ccc(Cl)nc2-c2ccc(B(O)O)c(C=O)c2)c2oc(C(C)C)c(C)c(=O)c2c1] 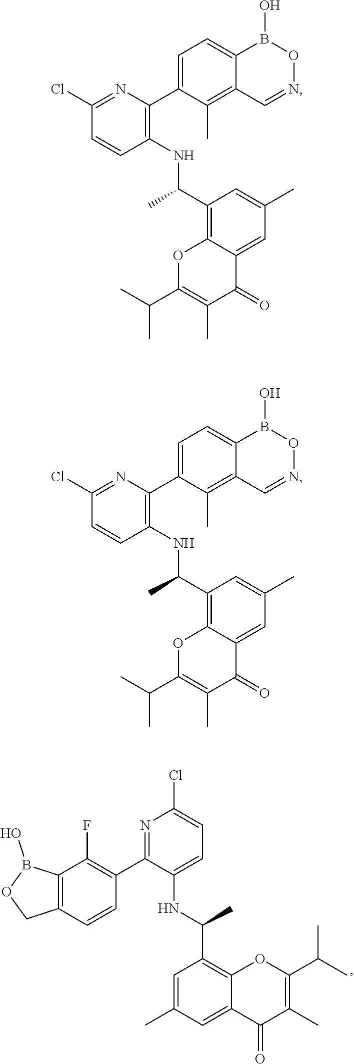 [SMILES: Cc1cc([C@H](C)Nc2ccc(Cl)nc2-c2ccc3c(c2C)C=NOB3O)c2oc(C(C)C)c(C)c(=O)c2c1 ; Cc1cc([C@@H](C)Nc2ccc(Cl)nc2-c2ccc3c(c2C)C=NOB3O)c2oc(C(C)C)c(C)c(=O)c2c1 ; Cc1cc([C@H](C)Nc2ccc(Cl)nc2-c2ccc3c(c2F)B(O)OC3)c2oc(C(C)C)c(C)c(=O)c2c1] 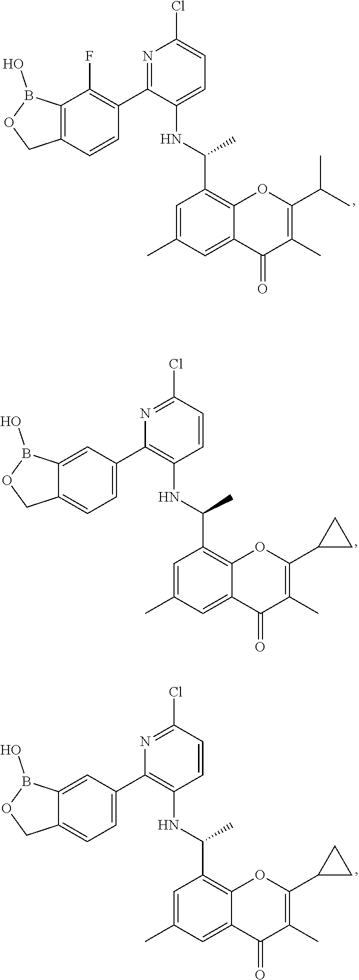 [SMILES: Cc1cc([C@@H](C)Nc2ccc(Cl)nc2-c2ccc3c(c2F)B(O)OC3)c2oc(C(C)C)c(C)c(=O)c2c1 ; Cc1cc([C@H](C)Nc2ccc(Cl)nc2-c2ccc3c(c2)B(O)OC3)c2oc(C3CC3)c(C)c(=O)c2c1 ; Cc1cc([C@@H](C)Nc2ccc(Cl)nc2-c2ccc3c(c2)B(O)OC3)c2oc(C3CC3)c(C)c(=O)c2c1] 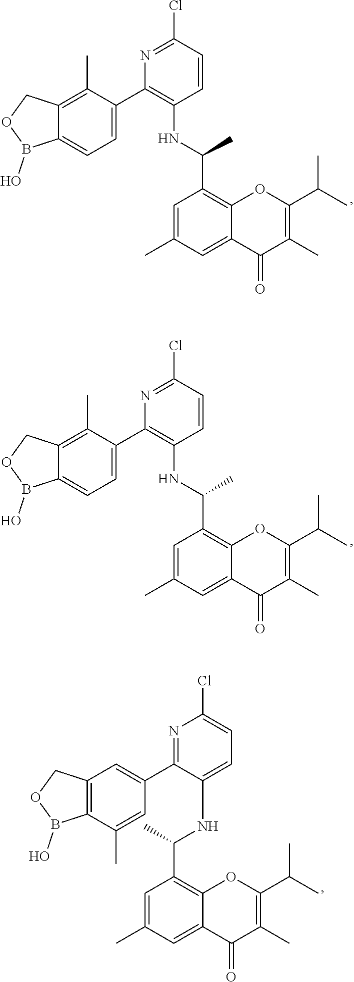 [SMILES: Cc1cc([C@H](C)Nc2ccc(Cl)nc2-c2ccc3c(c2C)COB3O)c2oc(C(C)C)c(C)c(=O)c2c1 ; Cc1cc([C@@H](C)Nc2ccc(Cl)nc2-c2ccc3c(c2C)COB3O)c2oc(C(C)C)c(C)c(=O)c2c1 ; Cc1cc([C@H](C)Nc2ccc(Cl)nc2-c2cc(C)c3c(c2)COB3O)c2oc(C(C)C)c(C)c(=O)c2c1] 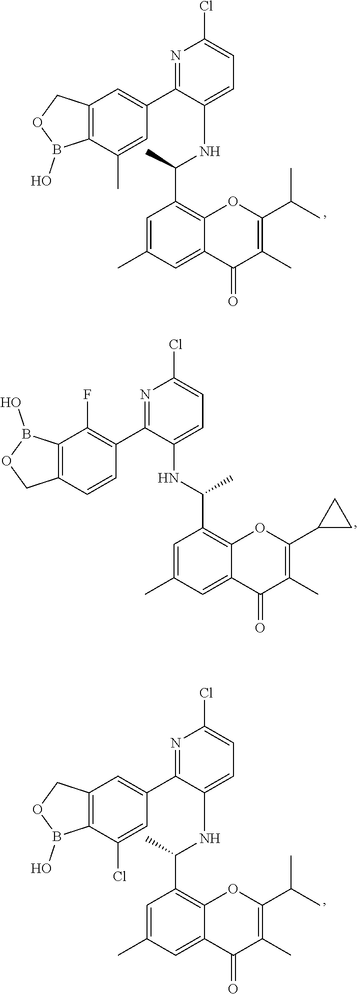 [SMILES: Cc1cc([C@@H](C)Nc2ccc(Cl)nc2-c2cc(C)c3c(c2)COB3O)c2oc(C(C)C)c(C)c(=O)c2c1 ; Cc1cc([C@@H](C)Nc2ccc(Cl)nc2-c2ccc3c(c2F)B(O)OC3)c2oc(C3CC3)c(C)c(=O)c2c1 ; Cc1cc([C@H](C)Nc2ccc(Cl)nc2-c2cc(Cl)c3c(c2)COB3O)c2oc(C(C)C)c(C)c(=O)c2c1] 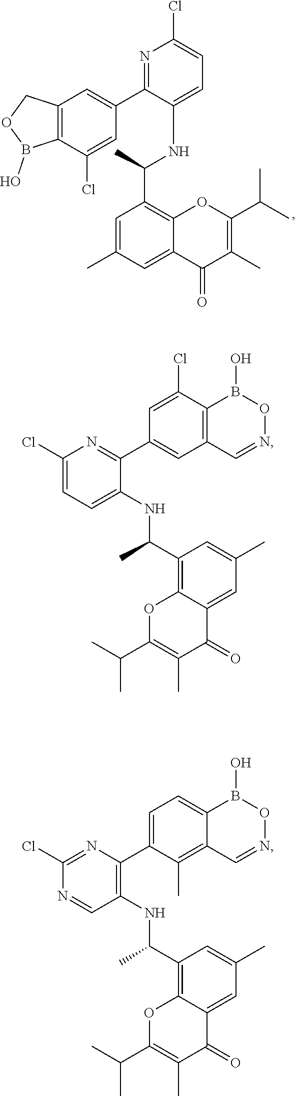 [SMILES: Cc1cc([C@@H](C)Nc2ccc(Cl)nc2-c2cc(Cl)c3c(c2)COB3O)c2oc(C(C)C)c(C)c(=O)c2c1 ; Cc1cc([C@@H](C)Nc2ccc(Cl)nc2-c2cc(Cl)c3c(c2)C=NOB3O)c2oc(C(C)C)c(C)c(=O)c2c1 ; Cc1cc([C@H](C)Nc2cnc(Cl)nc2-c2ccc3c(c2C)C=NOB3O)c2oc(C(C)C)c(C)c(=O)c2c1] 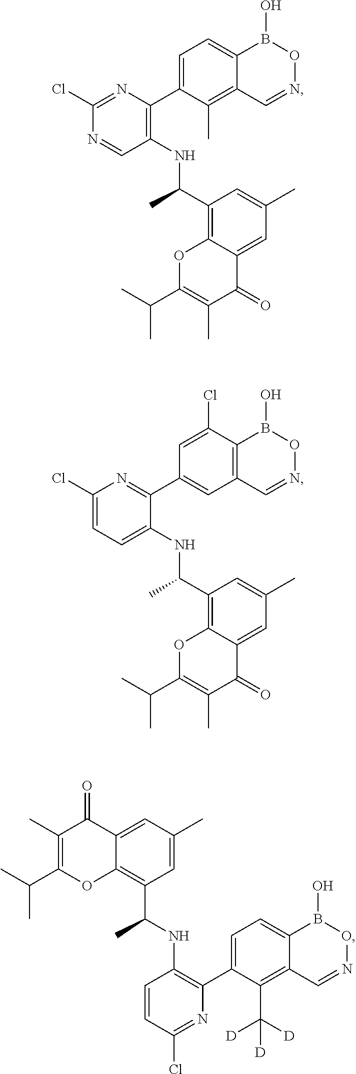 [SMILES: Cc1cc([C@@H](C)Nc2cnc(Cl)nc2-c2ccc3c(c2C)C=NOB3O)c2oc(C(C)C)c(C)c(=O)c2c1 ; Cc1cc([C@H](C)Nc2ccc(Cl)nc2-c2cc(Cl)c3c(c2)C=NOB3O)c2oc(C(C)C)c(C)c(=O)c2c1 ; [2H]C([2H])([2H])c1c(-c2nc(Cl)ccc2N[C@@H](C)c2cc(C)cc3c(=O)c(C)c(C(C)C)oc23)ccc2c1C=NOB2O] 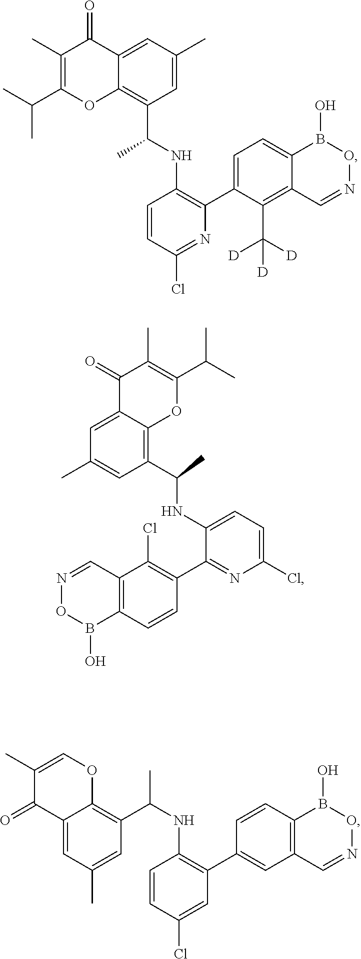 [SMILES: [2H]C([2H])([2H])c1c(-c2nc(Cl)ccc2N[C@H](C)c2cc(C)cc3c(=O)c(C)c(C(C)C)oc23)ccc2c1C=NOB2O ; Cc1cc([C@@H](C)Nc2ccc(Cl)nc2-c2ccc3c(c2Cl)C=NOB3O)c2oc(C(C)C)c(C)c(=O)c2c1 ; Cc1cc(C(C)Nc2ccc(Cl)cc2-c2ccc3c(c2)C=NOB3O)c2occ(C)c(=O)c2c1] 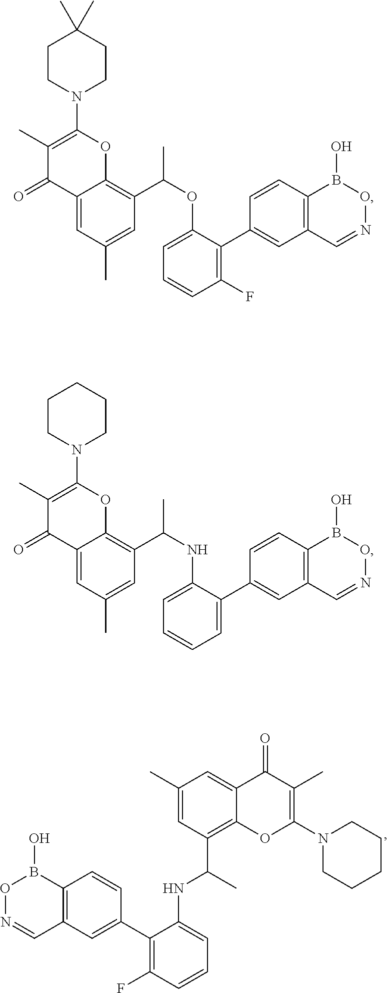 [SMILES: Cc1cc(C(C)Oc2cccc(F)c2-c2ccc3c(c2)C=NOB3O)c2oc(N3CCC(C)(C)CC3)c(C)c(=O)c2c1 ; Cc1cc(C(C)Nc2ccccc2-c2ccc3c(c2)C=NOB3O)c2oc(N3CCCCC3)c(C)c(=O)c2c1 ; Cc1cc(C(C)Nc2cccc(F)c2-c2ccc3c(c2)C=NOB3O)c2oc(N3CCCCC3)c(C)c(=O)c2c1] 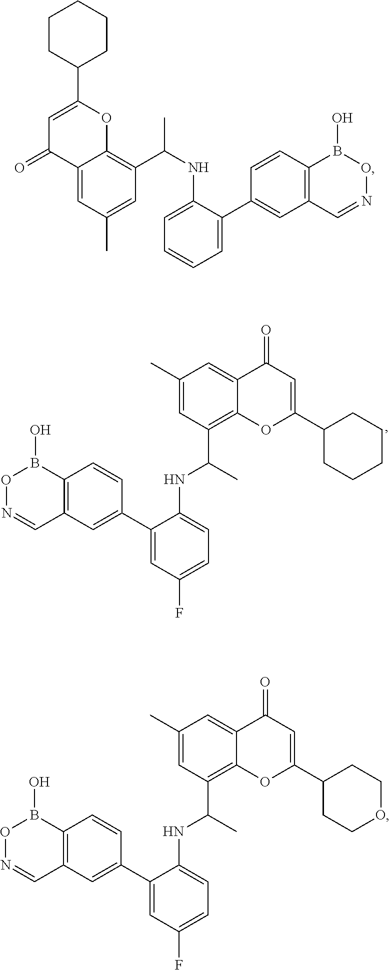 [SMILES: Cc1cc(C(C)Nc2ccccc2-c2ccc3c(c2)C=NOB3O)c2oc(C3CCCCC3)cc(=O)c2c1 ; Cc1cc(C(C)Nc2ccc(F)cc2-c2ccc3c(c2)C=NOB3O)c2oc(C3CCCCC3)cc(=O)c2c1 ; Cc1cc(C(C)Nc2ccc(F)cc2-c2ccc3c(c2)C=NOB3O)c2oc(C3CCOCC3)cc(=O)c2c1] 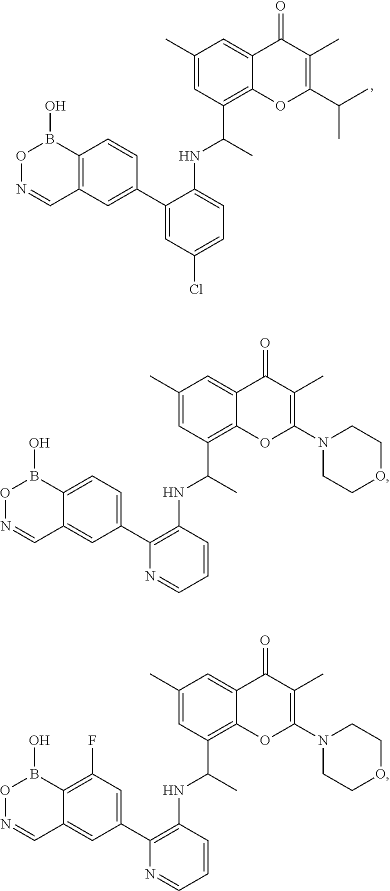 [SMILES: Cc1cc(C(C)Nc2ccc(Cl)cc2-c2ccc3c(c2)C=NOB3O)c2oc(C(C)C)c(C)c(=O)c2c1 ; Cc1cc(C(C)Nc2cccnc2-c2ccc3c(c2)C=NOB3O)c2oc(N3CCOCC3)c(C)c(=O)c2c1 ; Cc1cc(C(C)Nc2cccnc2-c2cc(F)c3c(c2)C=NOB3O)c2oc(N3CCOCC3)c(C)c(=O)c2c1] 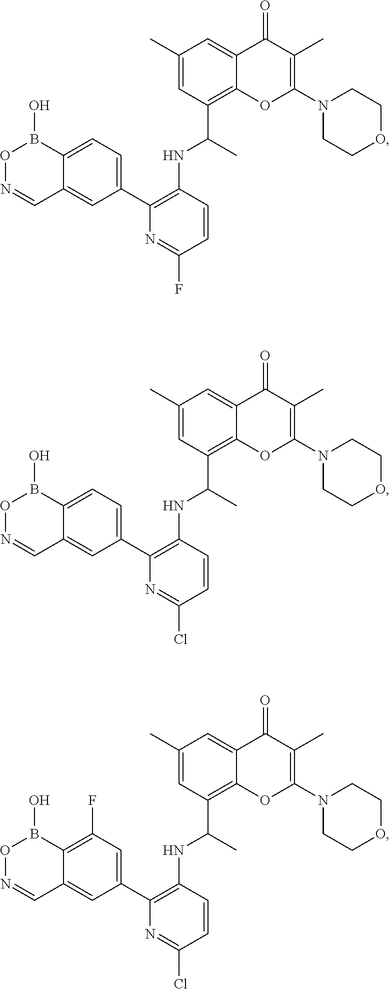 [SMILES: Cc1cc(C(C)Nc2ccc(F)nc2-c2ccc3c(c2)C=NOB3O)c2oc(N3CCOCC3)c(C)c(=O)c2c1 ; Cc1cc(C(C)Nc2ccc(Cl)nc2-c2ccc3c(c2)C=NOB3O)c2oc(N3CCOCC3)c(C)c(=O)c2c1 ; Cc1cc(C(C)Nc2ccc(Cl)nc2-c2cc(F)c3c(c2)C=NOB3O)c2oc(N3CCOCC3)c(C)c(=O)c2c1] 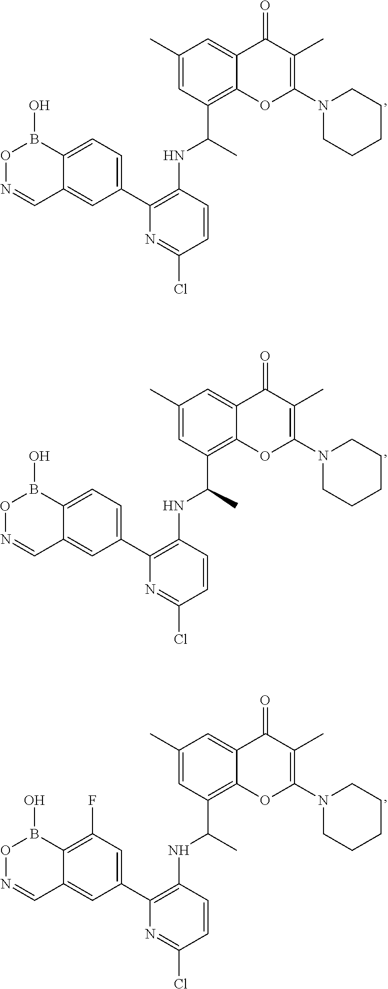 [SMILES: Cc1cc(C(C)Nc2ccc(Cl)nc2-c2ccc3c(c2)C=NOB3O)c2oc(N3CCCCC3)c(C)c(=O)c2c1 ; Cc1cc([C@@H](C)Nc2ccc(Cl)nc2-c2ccc3c(c2)C=NOB3O)c2oc(N3CCCCC3)c(C)c(=O)c2c1 ; Cc1cc(C(C)Nc2ccc(Cl)nc2-c2cc(F)c3c(c2)C=NOB3O)c2oc(N3CCCCC3)c(C)c(=O)c2c1] 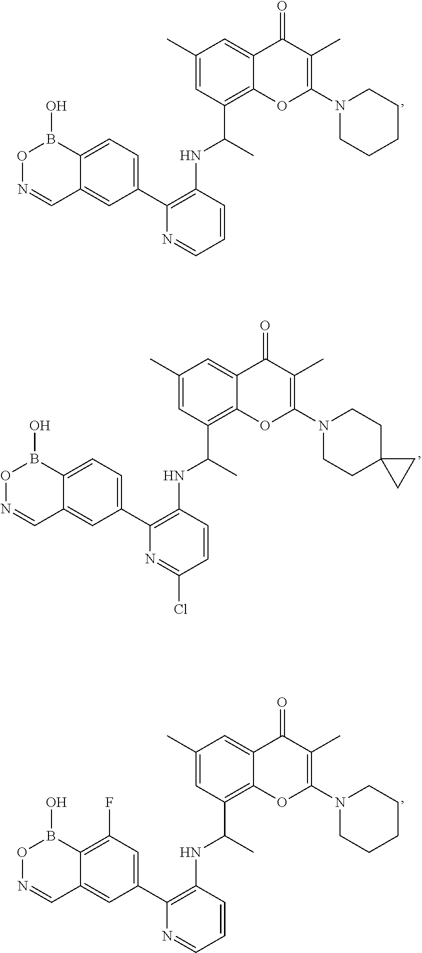 [SMILES: Cc1cc(C(C)Nc2cccnc2-c2ccc3c(c2)C=NOB3O)c2oc(N3CCCCC3)c(C)c(=O)c2c1 ; Cc1cc(C(C)Nc2ccc(Cl)nc2-c2ccc3c(c2)C=NOB3O)c2oc(N3CCC4(CC3)CC4)c(C)c(=O)c2c1 ; Cc1cc(C(C)Nc2cccnc2-c2cc(F)c3c(c2)C=NOB3O)c2oc(N3CCCCC3)c(C)c(=O)c2c1] 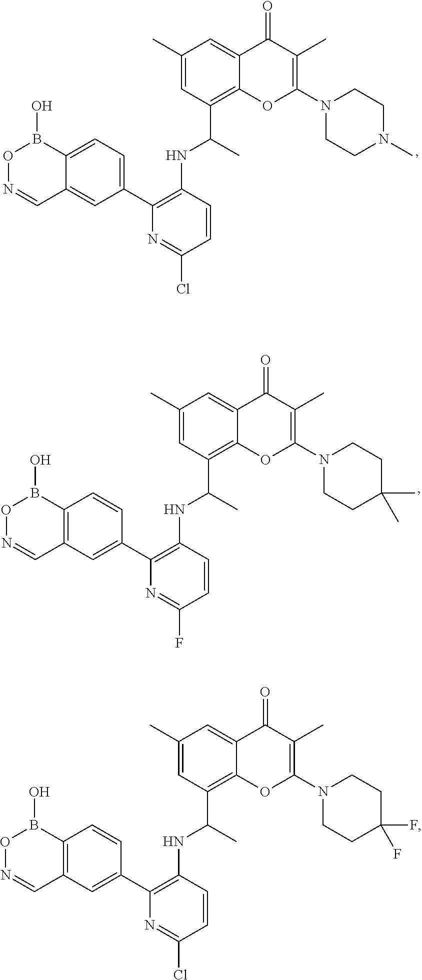 [SMILES: Cc1cc(C(C)Nc2ccc(Cl)nc2-c2ccc3c(c2)C=NOB3O)c2oc(N3CCN(C)CC3)c(C)c(=O)c2c1 ; Cc1cc(C(C)Nc2ccc(F)nc2-c2ccc3c(c2)C=NOB3O)c2oc(N3CCC(C)(C)CC3)c(C)c(=O)c2c1 ; Cc1cc(C(C)Nc2ccc(Cl)nc2-c2ccc3c(c2)C=NOB3O)c2oc(N3CCC(F)(F)CC3)c(C)c(=O)c2c1] 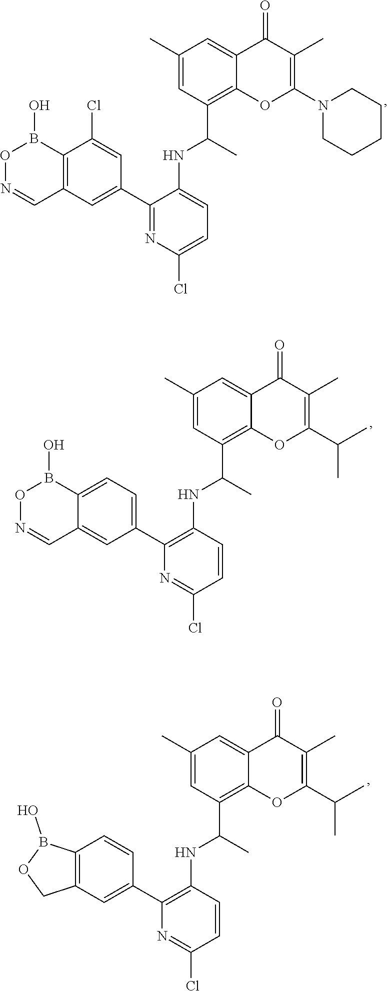 [SMILES: Cc1cc(C(C)Nc2ccc(Cl)nc2-c2cc(Cl)c3c(c2)C=NOB3O)c2oc(N3CCCCC3)c(C)c(=O)c2c1 ; Cc1cc(C(C)Nc2ccc(Cl)nc2-c2ccc3c(c2)C=NOB3O)c2oc(C(C)C)c(C)c(=O)c2c1 ; Cc1cc(C(C)Nc2ccc(Cl)nc2-c2ccc3c(c2)COB3O)c2oc(C(C)C)c(C)c(=O)c2c1] 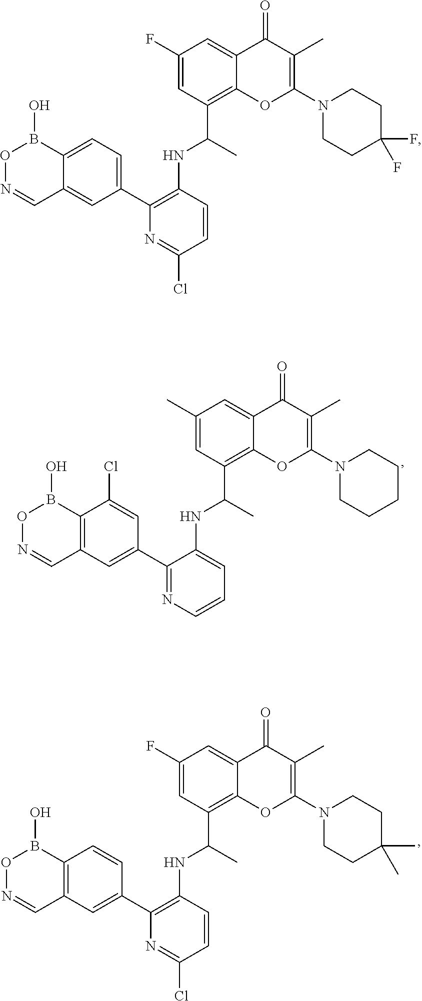 [SMILES: Cc1c(N2CCC(F)(F)CC2)oc2c(C(C)Nc3ccc(Cl)nc3-c3ccc4c(c3)C=NOB4O)cc(F)cc2c1=O ; Cc1cc(C(C)Nc2cccnc2-c2cc(Cl)c3c(c2)C=NOB3O)c2oc(N3CCCCC3)c(C)c(=O)c2c1 ; Cc1c(N2CCC(C)(C)CC2)oc2c(C(C)Nc3ccc(Cl)nc3-c3ccc4c(c3)C=NOB4O)cc(F)cc2c1=O] 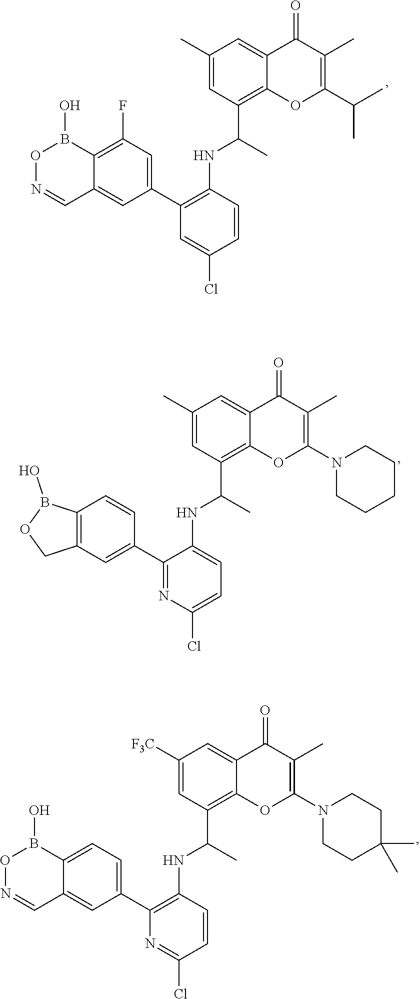 [SMILES: Cc1cc(C(C)Nc2ccc(Cl)cc2-c2cc(F)c3c(c2)C=NOB3O)c2oc(C(C)C)c(C)c(=O)c2c1 ; Cc1cc(C(C)Nc2ccc(Cl)nc2-c2ccc3c(c2)COB3O)c2oc(N3CCCCC3)c(C)c(=O)c2c1 ; Cc1c(N2CCC(C)(C)CC2)oc2c(C(C)Nc3ccc(Cl)nc3-c3ccc4c(c3)C=NOB4O)cc(C(F)(F)F)cc2c1=O] 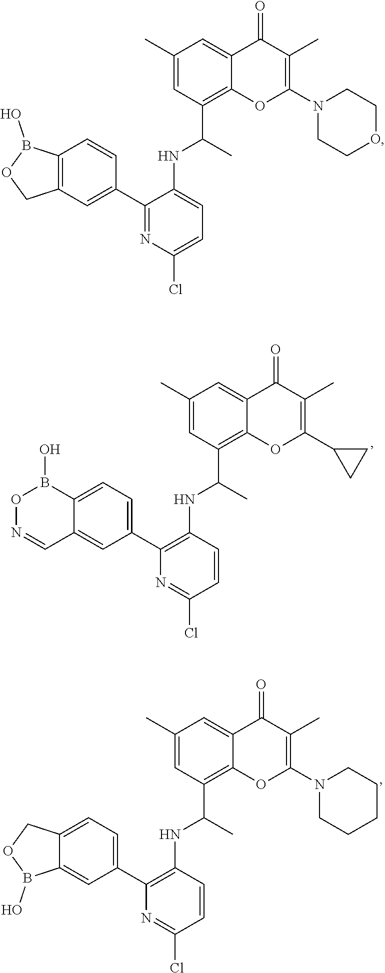 [SMILES: Cc1cc(C(C)Nc2ccc(Cl)nc2-c2ccc3c(c2)COB3O)c2oc(N3CCOCC3)c(C)c(=O)c2c1 ; Cc1cc(C(C)Nc2ccc(Cl)nc2-c2ccc3c(c2)C=NOB3O)c2oc(C3CC3)c(C)c(=O)c2c1 ; Cc1cc(C(C)Nc2ccc(Cl)nc2-c2ccc3c(c2)B(O)OC3)c2oc(N3CCCCC3)c(C)c(=O)c2c1] 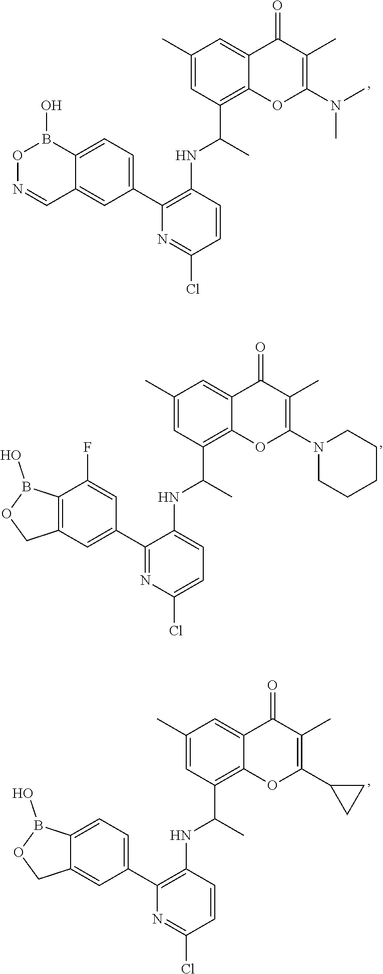 [SMILES: Cc1cc(C(C)Nc2ccc(Cl)nc2-c2ccc3c(c2)C=NOB3O)c2oc(N(C)C)c(C)c(=O)c2c1 ; Cc1cc(C(C)Nc2ccc(Cl)nc2-c2cc(F)c3c(c2)COB3O)c2oc(N3CCCCC3)c(C)c(=O)c2c1 ; Cc1cc(C(C)Nc2ccc(Cl)nc2-c2ccc3c(c2)COB3O)c2oc(C3CC3)c(C)c(=O)c2c1] 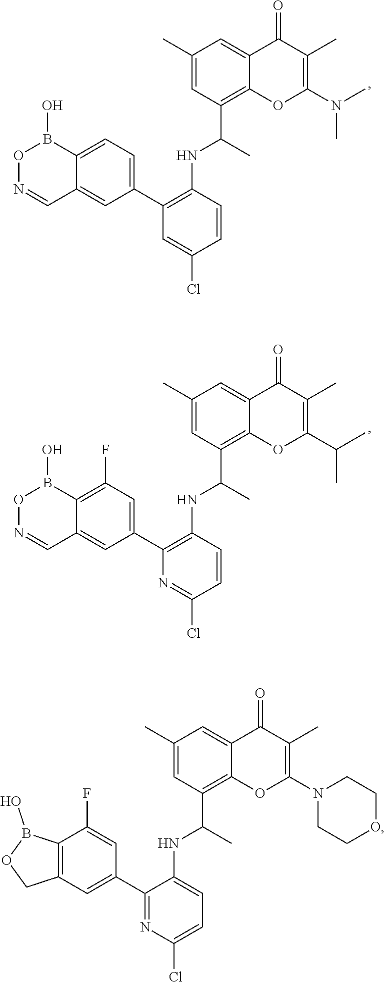 [SMILES: Cc1cc(C(C)Nc2ccc(Cl)cc2-c2ccc3c(c2)C=NOB3O)c2oc(N(C)C)c(C)c(=O)c2c1 ; Cc1cc(C(C)Nc2ccc(Cl)nc2-c2cc(F)c3c(c2)C=NOB3O)c2oc(C(C)C)c(C)c(=O)c2c1 ; Cc1cc(C(C)Nc2ccc(Cl)nc2-c2cc(F)c3c(c2)COB3O)c2oc(N3CCOCC3)c(C)c(=O)c2c1] 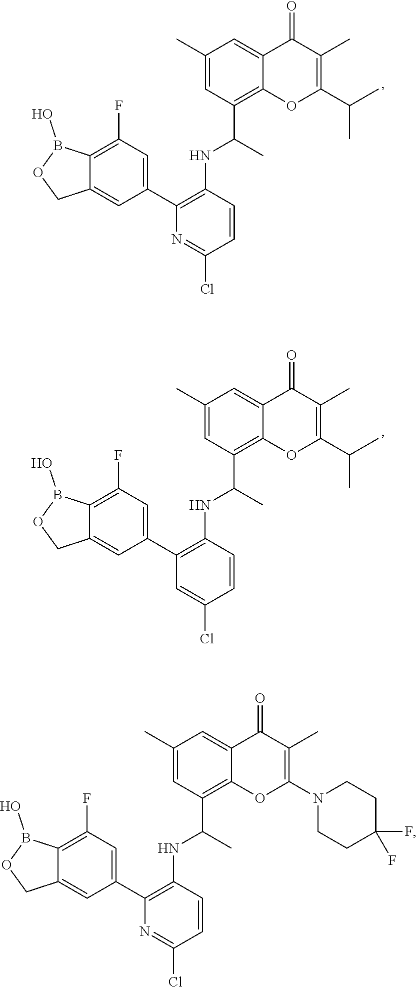 [SMILES: Cc1cc(C(C)Nc2ccc(Cl)nc2-c2cc(F)c3c(c2)COB3O)c2oc(C(C)C)c(C)c(=O)c2c1 ; Cc1cc(C(C)Nc2ccc(Cl)cc2-c2cc(F)c3c(c2)COB3O)c2oc(C(C)C)c(C)c(=O)c2c1 ; Cc1cc(C(C)Nc2ccc(Cl)nc2-c2cc(F)c3c(c2)COB3O)c2oc(N3CCC(F)(F)CC3)c(C)c(=O)c2c1] 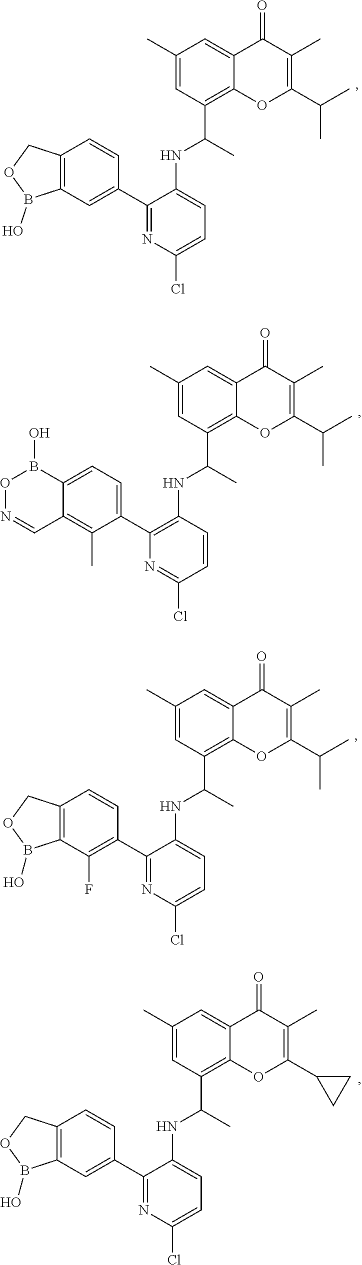 [SMILES: Cc1cc(C(C)Nc2ccc(Cl)nc2-c2ccc3c(c2)B(O)OC3)c2oc(C(C)C)c(C)c(=O)c2c1 ; Cc1cc(C(C)Nc2ccc(Cl)nc2-c2ccc3c(c2C)C=NOB3O)c2oc(C(C)C)c(C)c(=O)c2c1 ; Cc1cc(C(C)Nc2ccc(Cl)nc2-c2ccc3c(c2F)B(O)OC3)c2oc(C(C)C)c(C)c(=O)c2c1 ; Cc1cc(C(C)Nc2ccc(Cl)nc2-c2ccc3c(c2)B(O)OC3)c2oc(C3CC3)c(C)c(=O)c2c1] 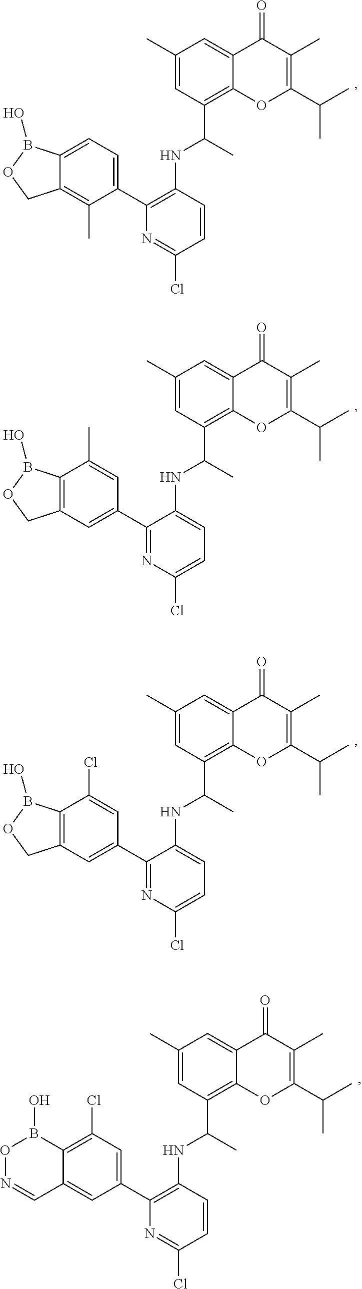 [SMILES: Cc1cc(C(C)Nc2ccc(Cl)nc2-c2ccc3c(c2C)COB3O)c2oc(C(C)C)c(C)c(=O)c2c1 ; Cc1cc(C(C)Nc2ccc(Cl)nc2-c2cc(C)c3c(c2)COB3O)c2oc(C(C)C)c(C)c(=O)c2c1 ; Cc1cc(C(C)Nc2ccc(Cl)nc2-c2cc(Cl)c3c(c2)COB3O)c2oc(C(C)C)c(C)c(=O)c2c1 ; Cc1cc(C(C)Nc2ccc(Cl)nc2-c2cc(Cl)c3c(c2)C=NOB3O)c2oc(C(C)C)c(C)c(=O)c2c1] 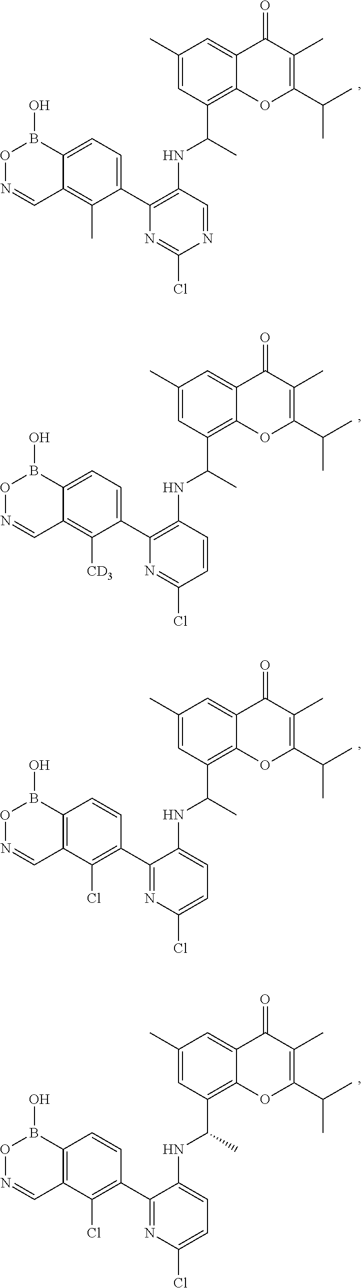 [SMILES: Cc1cc(C(C)Nc2cnc(Cl)nc2-c2ccc3c(c2C)C=NOB3O)c2oc(C(C)C)c(C)c(=O)c2c1 ; [2H]C([2H])([2H])c1c(-c2nc(Cl)ccc2NC(C)c2cc(C)cc3c(=O)c(C)c(C(C)C)oc23)ccc2c1C=NOB2O ; Cc1cc(C(C)Nc2ccc(Cl)nc2-c2ccc3c(c2Cl)C=NOB3O)c2oc(C(C)C)c(C)c(=O)c2c1 ; Cc1cc([C@H](C)Nc2ccc(Cl)nc2-c2ccc3c(c2Cl)C=NOB3O)c2oc(C(C)C)c(C)c(=O)c2c1] 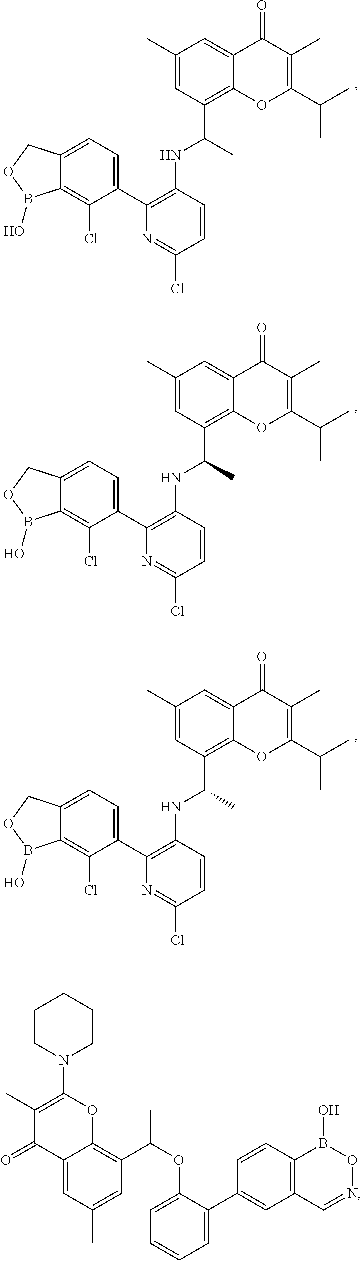 [SMILES: Cc1cc(C(C)Nc2ccc(Cl)nc2-c2ccc3c(c2Cl)B(O)OC3)c2oc(C(C)C)c(C)c(=O)c2c1 ; Cc1cc([C@@H](C)Nc2ccc(Cl)nc2-c2ccc3c(c2Cl)B(O)OC3)c2oc(C(C)C)c(C)c(=O)c2c1 ; Cc1cc([C@H](C)Nc2ccc(Cl)nc2-c2ccc3c(c2Cl)B(O)OC3)c2oc(C(C)C)c(C)c(=O)c2c1 ; Cc1cc(C(C)Oc2ccccc2-c2ccc3c(c2)C=NOB3O)c2oc(N3CCCCC3)c(C)c(=O)c2c1] 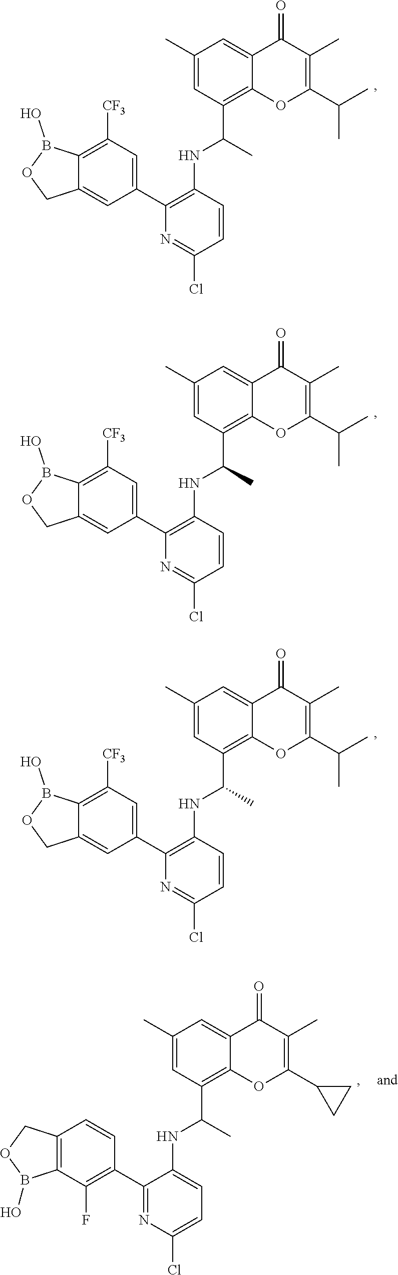 [SMILES: Cc1cc(C(C)Nc2ccc(Cl)nc2-c2cc3c(c(C(F)(F)F)c2)B(O)OC3)c2oc(C(C)C)c(C)c(=O)c2c1 ; Cc1cc([C@@H](C)Nc2ccc(Cl)nc2-c2cc3c(c(C(F)(F)F)c2)B(O)OC3)c2oc(C(C)C)c(C)c(=O)c2c1 ; Cc1cc([C@H](C)Nc2ccc(Cl)nc2-c2cc3c(c(C(F)(F)F)c2)B(O)OC3)c2oc(C(C)C)c(C)c(=O)c2c1 ; Cc1cc(C(C)Nc2ccc(Cl)nc2-c2ccc3c(c2F)B(O)OC3)c2oc(C3CC3)c(C)c(=O)c2c1] 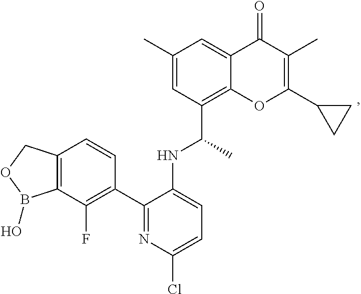 [SMILES: Cc1cc([C@H](C)Nc2ccc(Cl)nc2-c2ccc3c(c2F)B(O)OC3)c2oc(C3CC3)c(C)c(=O)c2c1]
or a tautomer, enantiomer, diastereomer, mixture, salt, hydrate, solvate, or deuterated form thereof.

152-155. (canceled)

156. A pharmaceutical composition comprising a therapeutically effective amount of a compound of claim 1, and a pharmaceutically acceptable excipient.

157. A method of inhibiting cell proliferation comprising contacting a cell with an effective amount of a compound of claim 1.

158. A method for treating cancer in a patient in need thereof, comprising administering a therapeutically effective amount of a compound of claim 1 to the patient.

159. A method of treating a PI3K-mediated disease or disorder in a patient in need of such treatment, the method comprising administering to the patient a therapeutically effective amount of a compound of claim 1.

160. A method of treating a disease or disorder mediated by one or more PIK3CA genes comprising modulating one or more of wild type or one or more mutations of the one or more PIK3CA genes.

161-189. (canceled)

190. A method for inhibiting PI3K activity in a cell, comprising modulating a solvent accessible surface area of a protein with a compound of claim 1.

191-219. (canceled)

220. A method of inhibiting PI3K comprising chemically capturing a solvent accessible surface area and modulating a p110 mutated protein subunit.

221-232. (canceled)

233. A method of treating a disease or disorder mediated by PI3Kα, comprising chemically capturing a solvent accessible amino acid residue and modulating one or more of H1047X, E545X, and E542X.

234-244. (canceled)

245. A method of inhibiting a PIK3CA gene target protein (PI3K) comprising modulating two or more mutant variants selected from mutations of H1047, E545, E542, N345, E726, C420, Q546, G118, E453, Q546, G1049, M1043, K111, E81, N1044, and E110.

246. (canceled)

247. A method of treating a disease or disorder mediated by PIK3CA, comprising modulating two or more mutant variants selected from mutations of H1047, E545, E542, N345, E726, C420, Q546, G118, E453, Q546, G1049, M1043, K111, E81, N1044, and E110.

248-249. (canceled)

250. A method of modulating a PI3K to treat a disease or disorder by interacting a compound of claim 1 with at least two mutant variants.

251-277. (canceled)

278. The method of claim 157 further comprising administration of one or more additional therapeutic agents.

279-283. (canceled)

284. A method to modulate one or more PI3K enzymes to regulate one or more of disease initiation and progression comprising interaction with at least one histidine and modulation of at least one surface accessible amino acid or residue.

285-288. (canceled)

289. A method for treating cancer in a patient in need thereof, comprising:
(a) determining that the cancer is associated with a PI3K wild-type or one or more PI3K mutations; and
(b) administering to the patient a therapeutically effective amount of a compound of claim 1.

290-302. (canceled)

303. A process for preparing a compound of claim 1.

304. (canceled)
